(19) United States
(12) Patent Application Publication
(10) Pub. No.: US 2024/0262806 A1
(43) Pub. Date: Aug. 8, 2024

(54) INHIBITORS OF NLRP3

(71) Applicant: PTC THERAPEUTICS, INC., South Plainfield, NJ (US)

(72) Inventors: XIAOYAN ZHANG, South Plainfield, NJ (US); TIANYI ZHENG, South Plainfield, NJ (US); SCOTT j BARRAZA, South Plainfield, NJ (US); LAUREN BEJCEK, South Plainfield, NJ (US); BRADLEY B GILBERT, South Plainfield, NJ (US); HUA GONG, South Plainfield, NJ (US); HANDOKO HANDOKO, South Plainfield, NJ (US); SEYEDMORTEZA HOSSEYNI, South Plainfield, NJ (US); EDUARDO HUARTE, South Plainfield, NJ (US); WOOHYUNG JEON, South Plainfield, NJ (US); JING LI, South Plainfield, NJ (US); YAO LIU, South Plainfield, NJ (US); KYLE NIEDERER, South Plainfield, NJ (US); MEENU PILLAI, South Plainfield, NJ (US); ERICA N PARKER, South Plainfield, NJ (US); ETTORE RASTELLI, South Plainfield, NJ (US); NADIYA SYDORENKO, South Plainfield, NJ (US); ANTHONY TURPOFF, South Plainfield, NJ (US); MATTHEW G WOLL, South Plainfield, NJ (US); NANJING ZHANG, South Plainfield, NJ (US); YAN ZHANG, South Plainfield, NJ (US); RAUFUL ALAM, South Plainfield, NJ (US)

(73) Assignee: PCT THERAPEUTICS, INC., South Plainfield, NJ (US)

(21) Appl. No.: 18/256,099

(22) PCT Filed: Aug. 24, 2022
(86) PCT No.: PCT/US22/75421

§ 371 Date: Jun. 6, 2023

Related U.S. Application Data
(60) Provisional application No. 63/237,049, filed on Aug. 25, 2021.
(60) Provisional application No. 63/311,463, filed on Feb. 18, 2022.

(51) Int. Cl. C07D 401/12; A61K 31/395; A61K 31/502; A61K 31/5025; A61K 31/53; A61K 31/5377; A61K 31/5383; A61K 31/55; A61K 31/551; A61K 31/553; C07D 237/34; C07D 401/14; C07D 403/12; C07D 405/14; C07D 413/12; C07D 417/14; C07D 471/04; C07D 487/04; C07D 491/048; C07D 495/04; C07D 498/04; C07D 519/00

(52) U.S. Cl. CPC C07D 401/12; A61K 31/502; A61K 31/5025; A61K 31/53; A61K 31/5377; A61K 31/5383; A61K 31/55; A61K 31/551; A61K 31/553; A61K 31/5545; C07D 237/34; C07D 401/14; C07D 403/12; C07D 405/14; C07D 413/12; C07D 417/14; C07D 471/04; C07D 487/04; C07D 491/048; C07D 495/04; C07D 498/04; C07D 519/00

(57) ABSTRACT

The present invention relates to novel compounds of Formulae I-XI: wherein each A, A′, Q, Q′, W, Rw, Y, and Z, and -- are as defined herein, which inhibit NOD-like receptor protein 3 (NLRP3) inflammasome activity. The invention further relates to the processes for their preparation, pharmaceutical compositions and medicaments containing them, and their use in the treatment of diseases and disorders mediated by NLRP3.

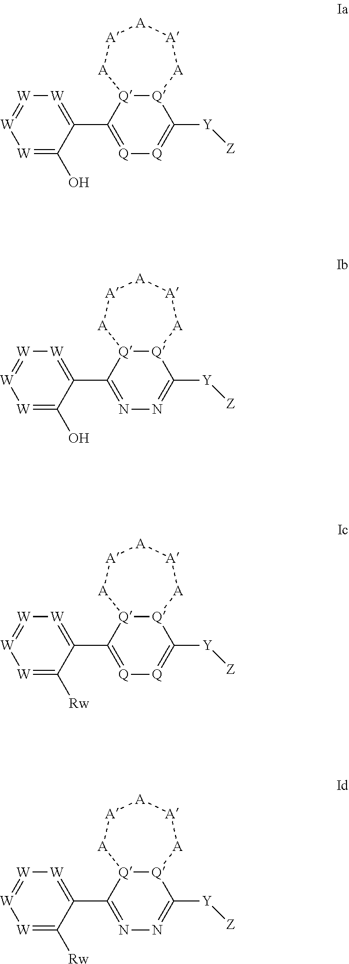

### CROSS-REFERENCE TO RELATED APPLICATIONS

This application claims the benefit of, and priority to U.S. Provisional Patent Application No. 63/237,049 filed on Aug. 25, 2021, and U.S. Provisional Patent Application No. 63/311,463 filed on Feb. 18, 2022, the contents of which are herein incorporated by reference in their entirety and for all purposes.

### FIELD OF THE INVENTION

The present invention relates to compounds that are useful as inhibitors of NOD-like receptor protein 3 (NLRP3) inflammasome pathway. The present invention also relates to processes for the preparation of said compounds, pharmaceutical compositions comprising said compounds, methods of using said compounds in the treatment of various diseases and disorders, and medicaments containing them, and their use in diseases and disorders mediated by NLRP3.

### BACKGROUND

The term of inflammasome was coined by Martinon et al. to describe the molecular platform triggering activation of inflammatory caspases and processing of interleukin 1 (IL-1) family cytokines (Fabio Martinon et al., Mol Cell 10(2):417-26, 2002). Inflammasomes are part of the innate immune system. Inflammasome activation is initiated by assembling of a multiprotein complex, including nucleotide binding oligomerization domain (NOD)-like receptor (NLR), the adapter apoptosis-associated speck-like protein containing a caspase recruitment domain (ASC), and the effector protease caspase-1. The assemble of the complex results in the activation of caspase-1 and the release of the mature proinflammatory cytokines, such as IL-1β and IL-18.

Among inflammasomes, the NLR family NACHT, LRR and PYD domains-containing protein 3 (NLRP3) inflammasome has been studied extensively and was found to be activated by a wide spectrum of stimuli. The regulatory mechanisms of NLRP3 activation are summarized in a recent review paper (Seungwha Paik et al., Cell Mol Immunol 18(5):1141-1160, 2021).

NLRP3 activation is triggered by various infectious, non-infectious molecules, including molecular byproducts of aging, physical inactivity and overnutrition. Once activated, it boosts the downstream production of the inflammatory cytokines IL-1β and IL-18. Gain-of function mutations of NLRP3 are associated with several genetic disorders including cryopyrin-associated periodic syndromes (CAPS). Additionally, NLRP3 is implicated in numerous common I) autoimmune, II) autoinflammatory, III) neurodegenerative, IV) cardiovascular and V) neuromuscular and muscular degenerative diseases e.g. (Matthew S J Mangan et al., Nat Rev Drug Discov 17(8):588-606, 2018; Corcoran et al., Pharmacol Rev 73(3):968-1000, 2021; Dubuisson et al., Cells 10(11):3023, 2021). Inflammasome activation has also been identified in retinal pigment epithelium (RPE) cells and proposed to be a causal factor for RPE dysfunction and degeneration (Gao et al., Mediators Inflamm 2015:690243, 2015). Further, NLRP3 activation is associated with severe COVID-19 cases and cytokine release syndrome (CRS) caused by cell-based therapeutics and biologic treatments (Tracey L Freeman and Talia H Swartz Front Immunol 11:1518, 2020; Lin et al., PLoS Pathog 6; 15(6):e1007795, 2019).

Therefore, an NLRP3 inflammasome inhibitor could be used as a single or combination of agents clinically as novel therapies for these diseases. Thus, there is a need for inhibitors of the NLRP3 inflammasome pathway to provide new and/or alternative treatments for these inflammasome-related diseases, disorders, such as autoinflammatory fever syndrome cryopyrin-associated periodic syndrome (CAPS), sickle cell disease, chronic liver disease, nonalcoholic steatohepatitis (NASH), gout, hyperoxaluria, pseudogout (chondrocalcinosis), Type I/Type II diabetes and related complications (e.g. nephropathy, retinopathy), fibrosis, rheumatoid arthritis, inflammatory bowel diseases, asthma and allergic airway inflammation, neuroinflammation-related disorders (e.g. multiple sclerosis, brain infection, acute injury, Alzheimer's disease, Parkinson's disease, Huntington's disease), neuromuscular and muscular degenerative diseases, atherosclerosis and cardiovascular risk (e.g. cardiovascular risk reduction (CvRR), hypertension), hidradenitis suppurativa, wound healing and scar formation, and cancer (e.g. colon cancer, lung cancer, myeloproliferative neoplasms, leukemias, myelodysplastic syndromes (MDS), myelofibrosis).

### REFERENCES

Fabio Martinon, Kimberly Burns, Jurg Tschopp The inflammasome: a molecular platform triggering activation of inflammatory caspases and processing of proIL-beta Mol Cell 10(2):417-26, 2002.
Seungwha Paik, Jin Kyung Kim, Prashanta Silwal, Chihiro Sasakawa, Eun-Kyeong Jo An update on the regulatory mechanisms of NLRP3 inflammasome activation Cell Mol Immunol 18(5):1141-1160, 2021.
Matthew S J Mangan, Edward J Olhava, William R Roush, H Martin Seidel, Gary D Glick, Eicke Latz Targeting the NLRP3 inflammasome in inflammatory diseases Nat Rev Drug Discov 17(8):588-606, 2018
Sarah Corcoran, Reena Halai, Matthew A Coope Pharmacological Inhibition of the Nod-Like Receptor Family Pyrin Domain Containing 3 Inflammasome with MCC950 Pharmacol Rev 73(3):968-1000, 2021
Nicolas Dubuisson, Romain Versele, Maria A Davis-López de Carrizosa, Camille M Selvais, Sonia M Brichard, Michel Abou-Samra Walking down Skeletal Muscle Lane: From Inflammasome to Disease Cells 10(11):3023, 2021
Jiangyuan Gao, Ruozhou Tom Liu, Sijia Cao, Jing Z Cui, Aikun Wang, Eleanor To, Joanne A Matsubara NLRP3 inflammasome: activation and regulation in age-related macular degeneration Mediators Inflamm 2015:690243, 2015
Tracey L Freeman and Talia H Swartz Targeting the NLRP3 Inflammasome in Severe COVID-19 Front Immunol 11:1518, 2020
Lan Lin, Lei Xu, Weihua Lv, Li Han, Yaozu Xiang, Lei Fu, Meilin Jin, Rui Zhou, Huanchun Chen, Anding Zhang An NLRP3 inflammasome-triggered cytokine storm contributes to Streptococcal toxic shock-like syndrome (STSLS) PLoS Pathog 6; 15(6):e1007795, 2019

### SUMMARY OF THE INVENTION

The invention provides compounds or pharmaceutically acceptable salts thereof, pharmaceutical compositions thereof, which compounds inhibit the NLRP3 inflammasome pathway. The invention further provides methods of treating, or preventing, disease and/or disorders related to NLRP3, comprising administering to a subject in need thereof an effective amount of the compounds of the invention, or a pharmaceutically acceptable salt thereof.

Various aspects of the invention are described herein.

The instant application discloses a compound of Formulae Ia, Ib, Ic, or Id:

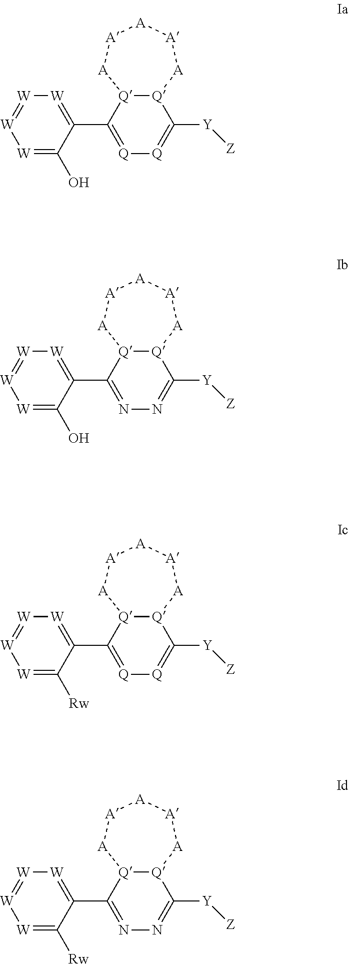

wherein:
R_{w} is hydrogen, C₁₋₆alkyl (e.g., CH₃), C₂₋₈alkynyl, halogen (e.g., F, Cl), C₁₋₆alkoxy (e.g., OCH₃), halo-C₁₋₄alkyl (e.g., CHF₂, CF₃), halo-C₁₋₄alkoxy (e.g., OCHF₂, OCF₃), cyano, —NH₂, —N(C₁₋₆alkyl)₂, thiol, —SO₂NH₂, —SO₂N(C₁₋₆alkyl)₂, —S(═O)(C₁₋₆alkyl), cycloalkyl, CHR₁ₐ, (C═O)R₁ₐ, OR₁ₐ, NR_{1b}, S(═O)R₁ₐ, S(═O)₂R₁ₐ, O(C═O)R₁ₐ, (C═O)OR₁ₐ, NR_{1b}(C═O)R_{1b}, (C═O)NR_{1b}, (C═O)N(R_{1b})₂ NR_{1b}(C═O)OR₁ₐ, O(C═O)NR_{1b}, ONR_{1b}(C═NR_{1b})NR_{1b}, NR_{1b}S(═O)₂ or S(═O)₂NR_{1b}, wherein each C₁₋₆alkyl, heterocycle (e.g., 2, 5-dihydrofuran-3-yl), heteroaryl and aryl are optionally substituted with 1 or 2 substituents each selected from R₅; each W is independently CH, CR′ or N;
each Q is independently N or CH;
each Q′ is independently N, C or CH;
each A is independently CH, CH₂, CRₐ, CHRₐ, CR₄, CHR₄, N, NH, NRₐ, NR₄, S, or O;
each A′ is independently absent, CH, CH₂, CRₐ, CHRₐ, CR₄, CHR₄, N, NH, NRₐ, NR₄, S, or O;
each Rₐ is independently hydrogen, deuterium, halogen, —CN, —OH, —OR₂, ═O, ═N—OR₂, —SR₂, —S(═O)R₂, —S(═O)₂R², —N(R₂)₂, —NR₂S(═O)(═NR₂)R², —NR₂S(═O)₂R², —S(═O)₂N(R₂)₂, —C(═O)R₂, —OC(═O)R₂, —C(═O)OR₂, —OC(═O)OR₂, —C(═O)N(R₂)₂, —OC(═O)N(R₂)₂, —NR₂C(═O)R₂, —P(═O)(R₂)₂, C₁₋₄alkyl, (C₁₋₄alkyl)₂, halo-C₁₋₆alkyl, C₁₋₆heteroalkyl, C₃₋₈cycloalkyl, C₂₋₇heterocycloalkyl, aryl or monocyclic heteroaryl;
each, [symbol] represents either an absent, single, or double bond;
Y is C(R₁ₐ)₂, C═O, O, NR_{1b}, or a bond;
each R₁ₐ is independently hydrogen, halogen, hydroxyl, cyano, C₁₋₄alkyl, deutero-C₁₋₄alkyl, halo-C₁₋₄alkyl, amino or hydroxy-C₁₋₄alkyl, C₃₋₁₀cycloalkyl, C₂₋₇heterocycloalkyl or aryl;
each R_{1b} is independently hydrogen, C₁₋₄alkyl, deutero-C₁₋₄alkyl, halo-C₁₋₄alkyl or hydroxy-C₁₋₄alkyl;
each R′ is independently heterocyclyl, heteroaryl, aryl, C₃₋₈cycloalkyl, C₁₋₄alkyl, C₁₋₄alkoxy, deutero-C₁₋₄alkyl, halo-C₁₋₄alkyl (e.g., trifluoromethyl, difluoromethyl), halo-C₁₋₄alkoxy (e.g., trifluoromethoxy), hydroxy-C₁₋₄alkyl, halogen, hydroxy or cyano, wherein each heterocyclyl (e.g., 1-methyl-2,5-dihydro-1H-pyrrol-3-yl, 2,5-dihydrofuran-3-yl, 3,6-dihydro-2H-pyran-3-yl, 3,6-dihydro-2H-pyran-4-yl, 5,6-dihydro-2H-pyran-3-yl, N-methyl pyrrol-3-yl, N-pyrrolidin-1-yl, pyrrolidin-3-yl, tetrahydro-2H-pyran-3-yl, tetrahydro-2H-pyran-4-yl, tetrahydrofuran-3-yl, N-methyl pyridinone-4-yl), heteroaryl (e.g., 1,2,3-triazol-1-yl, 1,2,3-triazol-2-yl, 1,2-pyrazol-4-yl, 5-methyl-1,2,4-thiadiazol-2-yl, N-methyl pyrrol-3-yl, N-methyl pyridinone-4-yl, tetrazol-5-yl), C₃₋₈cycloalkyl (2-cyanocycloprop-1-yl, cyclopropyl) and aryl (e.g., phenyl, 4-chloro-2-cyanophenyl, 4-chloro-2-hydroxyphenyl) are optionally substituted with 1, or 2 substituents each selected from R₃;
each Z is heterocyclyl, heteroaryl, aryl, C₃₋₁₀cycloalkyl, C₁₋₄alkyl, deutero-C₁₋₄alkyl, halo-C₁₋₄alkyl, hydroxy-C₁₋₈alkyl, C₁₋₆alkoxy, NH(hydroxy-C₁₋₆alkyl), NH(C₁₋₆alkoxy) wherein each Z is optionally substituted with OH, NH₂, —CO₂H, halogen, C₁₋₆alkyl, C₁₋₆haloalkyl, C₁₋₆ hydroxyalkyl, C₂₋₆acyl, C₂₋₆alkanoic acid, C₂₋₆alkanoate ester, or heterocyclyl, and wherein heterocyclyl, C₃₋₁₀cycloalkyl is a saturated or partially unsaturated 3-7 membered monocyclic, 6-10 membered bicyclic or 13-16 membered polycyclic ring system having 1, 2, or 3 heteroatom ring members independently selected from N, O, and S, each optionally substituted with 1, 2, 3, 4, or 5 substituents each selected from R₂;
R₂ is independently selected from halogen, hydroxyl, cyano, C₁₋₄alkyl, hydroxy-C₁₋₄alkyl, deuterium-C₁₋₄alkyl, halo-C₁₋₄alkyl, amino, C₁₋₄alkyl-amino, (C₁₋₆alkyl)₂-amino, halo-C₁₋₄alkyl-amino, (halo-C₁₋₆alkyl)₂-amino, hydroxy-C₁₋₄alkyl-amino, C₁₋₄alkoxy-C₁₋₄alkyl-amino, amino-C₁₋₄alkyl, C₁₋₄alkyl-amino-C₁₋₄alkyl, (C₁₋₄alkyl-amino)₂-C₁₋₄alkyl, C₁₋₄alkoxy, halo-C₁₋₄alkoxy, hydroxy-C₁₋₄alkoxy, C₁₋₄alkyl-C₁₋₄alkoxy, C₃₋₁₀cycloalkyl, C₃₋₁₀cycloalkyl-amino, C₃₋₁₀cycloalkyl-amino-C₁₋₄alkyl, heteroaryl-C₁₋₄alkyl, heteroaryl-amino, heteroaryl-C₁₋₄alkyl-amino, heterocyclyl, heterocyclyl-C₁₋₄alkyl, heterocyclyl-amino, heterocyclyl-amino-C₁₋₄alkyl, heterocyclyl-C₁₋₄alkoxy, heterocyclyl-amino-C₃₋₁₀cycloalkyl, phenyl, and phenyl-C₁₋₄alkoxy,
wherein heteroaryl is a 5-6 membered monocyclic or 6-10 membered bicyclic ring system having 1, 2, 3, or 4 heteroatom ring members independently selected from N, O, or S,
wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S,
wherein C₃₋₁₀cycloalkyl is a saturated or partially unsaturated 3-7 membered monocyclic ring system, and
wherein each instance of phenyl, heteroaryl, heterocyclyl, or C₃₋₁₀cycloalkyl is optionally substituted with 1 or 2 substituents each selected from R₃;
R₃ is independently selected from halogen, hydroxyl, cyano, C₁₋₄alkyl, deutero-C₁₋₄alkyl, halo-C₁₋₄alkyl, amino, C₁₋₄alkoxy, and halo-C₁₋₄alkoxy;
each R₄ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, halo-C₁₋₄alkoxy, heteroaryl, heterocyclyl, and phenyl,
wherein heteroaryl is a 5-6 membered monocyclic or 9-10 membered bicyclic ring system having 1, 2, 3, or 4 heteroatom ring members selected from N, O, and S,
wherein heterocyclyl is a saturated or partially unsaturated 3-7 membered monocyclic or 9-10 membered bicyclic ring system having 1, 2, or 3 heteroatom ring members selected from N, O, and S, and
wherein each instance of phenyl, heteroaryl or heterocyclyl is optionally substituted with 1, or 2 substituents each selected from R₅;
R₅ is independently selected from halogen, hydroxyl, cyano, nitro, C₁₋₄alkyl, deutero-C₁₋₄alkyl, halo-C₁₋₄alkyl, amino, C₁₋₄alkyl-amino, (C₁₋₄alkyl)₂-amino, amino-C₁₋₄alkyl, hydroxy-C₁₋₄alkyl, C₁₋₄alkyl-carbonyl, C₁₋₄alkoxy, C₁₋₄alkylthio, halo-C₁₋₄alkoxy, and C₃₋₁₀cycloalkyl; with the proviso that the compound is not Phenol, 5-([1,1′-biphenyl]-4-yl-4-dibenzothienylamino)-2-[4-([1,1′-biphenyl]-4-yl-4-dibenzothienylamino)-1-naphthalenyl]; 5-[Phenyl(6-phenyl-4-dibenzothienyl)amino]-2-[4-[phenyl(6-phenyl-4-dibenzothienyl)amino]-1-naphthalenyl]phenol; 5-[2-Dibenzothienyl(9,9-dimethyl-9H-fluoren-2-yl)amino]-2-[4-[2-dibenzothienyl(9,9-dimethyl-9H-fluoren-2-yl)amino]-1-naphthalenyl]phenol; 5-[2-Dibenzofuranyl(9,9-dimethyl-9H-fluoren-2-yl)amino]-2-[4-[2-dibenzofuranyl(9,9-dimethyl-9H-fluoren-2-yl)amino]-1-naphthalenyl]phenol; 5-(4-Dibenzofuranylphenylamino)-2-[4-(4-dibenzofuranylphenylamino)-1-naphthalenyl]phenol; 5-(4-Dibenzofuranyl-2-naphthalenylamino)-2-[4-(4-dibenzofuranyl-2-naphthalenylamino)-1-naphthalenyl]phenol; 5-[Phenyl(6-phenyl-4-dibenzofuranyl)amino]-2-[4-[phenyl(6-phenyl-4-dibenzofuranyl)amino]-1-naphthalenyl]phenol; 5-([1,1′-Biphenyl]-2-yl-4-dibenzofuranylamino)-2-[4-([1,1′-biphenyl]-2-yl-4-dibenzofuranylamino)-1-naphthalenyl]phenol; 4-[4-(4-Dibenzofuranylphenylamino)-1-naphthalenyl]-2′,3′,5′,6′-tetrafluoro-4′-(2-naphthalenylphenylamino)[1,1′-biphenyl]-3-ol; 2-[4-[[6-[[3-(2-Amino-4-pyrimidinyl)-2-pyridinyl]oxy]-3-pyridinyl]amino]-1-phthalazinyl]phenol; or 2-[4-[[6-[[3-(2-Amino-4-pyrimidinyl)-2-pyridinyl]oxy]-3-pyridinyl]amino]-1-phthalazinyl]-4-fluorophenol; and
wherein a form of the compound is selected from the group consisting of a pharmaceutically acceptable salt, hydrate, solvate, racemate, enantiomer, diastereomer, stereoisomer, and tautomer form thereof.

The invention provides a pharmaceutical composition comprising a therapeutically effective amount of a compound according to the definition of the compound of Formulae I-XI, or subFormulae thereof, as disclosed herein, or a pharmaceutically acceptable salt thereof, and one or more pharmaceutically acceptable carriers. The pharmaceutical composition is useful in the treatment of diseases and/or disorders related to the NLRP3 activity. In another aspect, the invention provides a combination, in particular a pharmaceutical combination, comprising a therapeutically effective amount of a compound according to the definition of compounds of Formulae I-XI, or subFormulae thereof, as disclosed herein, or a pharmaceutically acceptable salt thereof, and one or more therapeutic agents. In another aspect, the invention provides a combination, in particular a pharmaceutical combination, as disclosed herein, for use as a medicament.

In another aspect, the invention provides a compound of Formulae I-XI, or subFormulae thereof, as disclosed herein, or a pharmaceutically acceptable salt thereof, for use in the treatment of a disease or disorder in which the NLRP3 signaling contributes to the pathology, and/or symptoms, and/or progression, of said disease or disorder. In another aspect, the invention provides a method of treating a disease or disorder in which the NLRP3 signaling contributes to the pathology, and/or symptoms, and/or progression, of said disease or disorder, comprising administering a therapeutically effective amount of a compound of Formulae I-XI, or subFormulae thereof, as disclosed herein, or a pharmaceutically acceptable salt thereof. In another aspect, the invention provides a method of inhibiting the NLRP3 inflammasome activity in a subject in need thereof, the method comprises administering to the subject in need thereof a therapeutically effective amount of a compound of Formulae I-XI, or subFormulae thereof, as disclosed herein, or a pharmaceutically acceptable salt thereof. Another aspect of the invention, relates to the use of a compound of Formulae I-XI, or subFormulae thereof, as disclosed herein, or a pharmaceutically acceptable salt thereof, as a medicament.

Another aspect of the invention, relates to a compound of Formulae I-XI, or subFormulae thereof, as disclosed herein, or a pharmaceutically acceptable salt thereof, for use as a medicament.

Another aspect of the invention, also provides a compound of Formulae I-XI, or subFormulae thereof, as disclosed herein, or a pharmaceutically acceptable salt thereof, for use in the treatment of a disease or disorder selected from inflammasome-related disease/disorders, immune diseases, inflammatory diseases, auto-immune diseases, and auto-inflammatory diseases.

### DETAILED DESCRIPTION OF THE INVENTION

An aspect of the invention provides a compound having the structure of Formulae Ia, Ib, Ic, or Id:

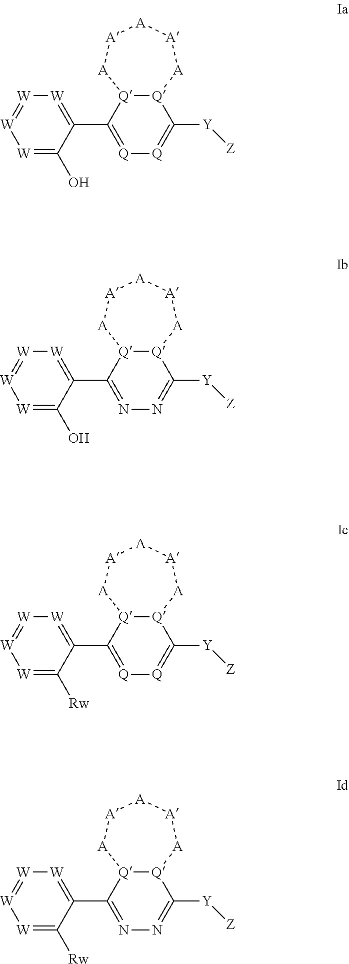

wherein:
R_{w} is hydrogen, C₁₋₆alkyl, C₂₋₈alkynyl, halogen, C₁₋₆alkoxy, halo-C₁₋₄alkyl, halo-C₁₋₄ alkoxy, cyano, —NH₂, —N(C₁₋₆alkyl)₂, thiol, —SO₂NH₂, —SO₂N(C₁₋₆alkyl)₂, —S(═O)(C₁₋₆alkyl), cycloalkyl, CHR₁ₐ, (C═O)R₁ₐ, OR₁ₐ, N(R_{1b})₂, S(═O)R₁ₐ, S(═O)₂R₁ₐ, O(C═O)R₁ₐ, (C═O)OR₁ₐ, NR_{1b}(C═O)R_{1b}, (C═O)NHR_{1b}, (C═O)N(R_{1b})₂, NR_{1b}(C═O)OR₁ₐ, O(C═O)N(R_{1b})₂, ONR_{1b}(C═NR_{1b})NR_{1b}, NR_{1b}S(═O)₂R₁ₐ or S(═O)₂N(R_{1b})₂, wherein each C₁₋₆alkyl, heterocycle, heteroaryl and aryl are optionally substituted with 1 or 2 substituents each selected from R₅;
each W is independently CH, CR′ or N;
each Q is independently N or CH;
each Q′ is independently N, C or CH;
each A is independently CH, CH₂, CRₐ, CHRₐ, CR₄, CHR₄, N, NH, NRₐ, NR₄, S, or O;
each A′ is independently absent, CH, CH₂, CRₐ, CHRₐ, CR₄, CHR₄, N, NH, NRₐ, NR₄, S, or O;
each Rₐ is independently H, deuterium, halogen, —CN, —OH, —OR₂, ═O, ═N—OR₂, —SR₂, —S(═O)R₂, —S(═O)₂R², —N(R₂)₂, —NR₂S(═O)(═NR₂)R², —NR₂S(═O)₂R², —S(═O)₂N(R₂)₂, —C(═O)R₂, —OC(═O)R₂, —C(═O)OR₂, —OC(═O)OR₂, —C(═O)N(R₂)₂, —OC(═O)N(R₂)₂, —NR₂C(═O)R₂, —P(═O)(R₂)₂, C₁₋₄alkyl, (C₁₋₄alkyl)₂, halo-C₁₋₆alkyl, C₁₋₆heteroalkyl, C₃₋₈cycloalkyl, C₂₋₇heterocycloalkyl, aryl or monocyclic heteroaryl;
each [symbol] represents either an absent, single, or double bond;
Y is C(R₁ₐ)₂, C═O, O, NR_{1b}, or a bond;
each R₁ₐ is independently hydrogen, halogen, hydroxyl, cyano, C₁₋₄alkyl, deutero-C₁₋₄alkyl, halo-C₁₋₄alkyl, amino or hydroxy-C₁₋₄alkyl, C₃₋₁₀cycloalkyl, C₂₋₇heterocycloalkyl or aryl;
each R_{1b} is independently hydrogen, C₁₋₄alkyl, deutero-C₁₋₄alkyl, halo-C₁₋₄alkyl or hydroxy-C₁₋₄alkyl;
each R′ is independently heterocyclyl, heteroaryl, aryl, C₃₋₈cycloalkyl, C₁₋₄alkyl, C₁₋₄alkoxy, deutero-C₁₋₄alkyl, halo-C₁₋₄alkyl, halo-C₁₋₄alkoxy, hydroxy-C₁₋₄alkyl, halogen, hydroxy or cyano,
wherein each heterocycle, heteroaryl, C₃₋₈cycloalkyl and aryl are optionally substituted with 1, or 2 substituents each selected from R₃;
each Z is heterocyclyl, heteroaryl, aryl, C₃₋₁₀cycloalkyl, C₁₋₄alkyl, deutero-C₁₋₄alkyl, halo-C₁₋₄alkyl, hydroxy-C₁₋₈alkyl, C₁₋₆alkoxy, NH(hydroxy-C₁₋₆alkyl), NH(C₁₋₆alkoxy) wherein each Z is optionally substituted with OH, NH₂, —CO₂H, halogen, C₁₋₆alkyl, C₁₋₆haloalkyl, C₁₋₆ hydroxyalkyl, C₂₋₆acyl, C₂₋₆alkanoic acid, C₂₋₆alkanoate ester, or heterocyclyl, and wherein heterocyclyl, C₃₋₁₀cycloalkyl is a saturated or partially unsaturated 3-7 membered monocyclic, 6-10 membered bicyclic or 13-16 membered polycyclic ring system having 1, 2, or 3 heteroatom ring members independently selected from N, O, and S, each optionally substituted with 1, 2, 3, 4, or 5 substituents each selected from R₂;
R₂ is independently selected from halogen, hydroxyl, cyano, C₁₋₄alkyl, hydroxy-C₁₋₄alkyl, deuterium-C₁₋₄alkyl, halo-C₁₋₄alkyl, amino, C₁₋₄alkyl-amino, (C₁₋₆alkyl)₂-amino, halo-C₁₋₄alkyl-amino, (halo-C₁₋₆alkyl)₂-amino, hydroxy-C₁₋₄alkyl-amino, C₁₋₄alkoxy-C₁₋₄alkyl-amino, amino-C₁₋₄alkyl, C₁₋₄alkyl-amino-C₁₋₄alkyl, (C₁₋₄alkyl-amino)₂-C₁₋₄alkyl, C₁₋₄alkoxy, halo-C₁₋₄alkoxy, hydroxy-C₁₋₄alkoxy, C₁₋₄alkyl-C₁₋₄alkoxy, C₃₋₁₀cycloalkyl, C₃₋₁₀cycloalkyl-amino, C₃₋₁₀cycloalkyl-amino-C₁₋₄alkyl, heteroaryl-C₁₋₄alkyl, heteroaryl-amino, heteroaryl-C₁₋₄alkyl-amino, heterocyclyl, heterocyclyl-C₁₋₄alkyl, heterocyclyl-amino, heterocyclyl-amino-C₁₋₄alkyl, heterocyclyl-C₁₋₄alkoxy, heterocyclyl-amino-C₃₋₁₀cycloalkyl, phenyl, and phenyl-C₁₋₄alkoxy,
wherein heteroaryl is a 5-6 membered monocyclic or 6-10 membered bicyclic ring system having 1, 2, 3, or 4 heteroatom ring members independently selected from N, O, and S,
wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S,
wherein C₃₋₁₀cycloalkyl is a saturated or partially unsaturated 3-7 membered monocyclic ring system, and
wherein each instance of phenyl, heteroaryl, heterocyclyl, or C₃₋₁₀cycloalkyl is optionally substituted with 1 or 2 substituents each selected from R₃;
R₃ is independently selected from halogen, hydroxyl, cyano, C₁₋₄alkyl, deutero-C₁₋₄alkyl, halo-C₁₋₄alkyl, amino, C₁₋₄alkoxy, and halo-C₁₋₄alkoxy;
each R₄ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, halo-C₁₋₄alkoxy, heteroaryl, heterocyclyl, and phenyl,
wherein heteroaryl is a 5-6 membered monocyclic or 9-10 membered bicyclic ring system having 1, 2, 3, or 4 heteroatom ring members selected from N, O, and S,
wherein heterocyclyl is a saturated or partially unsaturated 3-7 membered monocyclic or 9-10 membered bicyclic ring system having 1, 2, or 3 heteroatom ring members selected from N, O, and S, and
wherein each instance of phenyl, heteroaryl or heterocyclyl is optionally substituted with 1, or 2 substituents each selected from R₅;
R₅ is independently selected from halogen, hydroxyl, cyano, nitro, C₁₋₄alkyl, deutero-C₁₋₄alkyl, halo-C₁₋₄alkyl, amino, C₁₋₄alkyl-amino, (C₁₋₄alkyl)₂-amino, amino-C₁₋₄alkyl, hydroxy-C₁₋₄alkyl, C₁₋₄alkyl-carbonyl, C₁₋₄alkoxy, C₁₋₄alkylthio, halo-C₁₋₄alkoxy, and C₃₋₁₀cycloalkyl;
with the proviso that the compound is not Phenol, 5-([1,1′-biphenyl]-4-yl-4-dibenzothienylamino)-2-[4-([1,1′-biphenyl]-4-yl-4-dibenzothienylamino)-1-naphthalenyl]; 5-[Phenyl(6-phenyl-4-dibenzothienyl)amino]-2-[4-[phenyl(6-phenyl-4-dibenzothienyl)amino]-1-naphthalenyl]phenol; 5-[2-Dibenzothienyl(9,9-dimethyl-9H-fluoren-2-yl)amino]-2-[4-[2-dibenzothienyl(9,9-dimethyl-9H-fluoren-2-yl)amino]-1-naphthalenyl]phenol; 5-[2-Dibenzofuranyl(9,9-dimethyl-9H-fluoren-2-yl)amino]-2-[4-[2-dibenzofuranyl(9,9-dimethyl-9H-fluoren-2-yl)amino]-1-naphthalenyl]phenol; 5-(4-Dibenzofuranylphenylamino)-2-[4-(4-dibenzofuranylphenylamino)-1-naphthalenyl]phenol; 5-(4-Dibenzofuranyl-2-naphthalenylamino)-2-[4-(4-dibenzofuranyl-2-naphthalenylamino)-1-naphthalenyl]phenol; 5-[Phenyl(6-phenyl-4-dibenzofuranyl)amino]-2-[4-[phenyl(6-phenyl-4-dibenzofuranyl)amino]-1-naphthalenyl]phenol; 5-([1,1′-Biphenyl]-2-yl-4-dibenzofuranylamino)-2-[4-([1,1′-biphenyl]-2-yl-4-dibenzofuranylamino)-1-naphthalenyl]phenol; 4-[4-(4-Dibenzofuranylphenylamino)-1-naphthalenyl]-2′,3′,5′,6′-tetrafluoro-4′-(2-naphthalenylphenylamino)[1,1′-biphenyl]-3-ol; 2-[4-[[6-[[3-(2-Amino-4-pyrimidinyl)-2-pyridinyl]oxy]-3-pyridinyl]amino]-1-phthalazinyl]phenol; or 2-[4-[[6-[[3-(2-Amino-4-pyrimidinyl)-2-pyridinyl]oxy]-3-pyridinyl]amino]-1-phthalazinyl]-4-fluorophenol; and
wherein a form of the compound is selected from the group consisting of a pharmaceutically acceptable salt, hydrate, solvate, racemate, enantiomer, diastereomer, stereoisomer, tautomer, and isotope enriched form thereof.

Another aspect of the invention provides a compound having the structure of Formulae IIa, IIb, IIc, or IId:

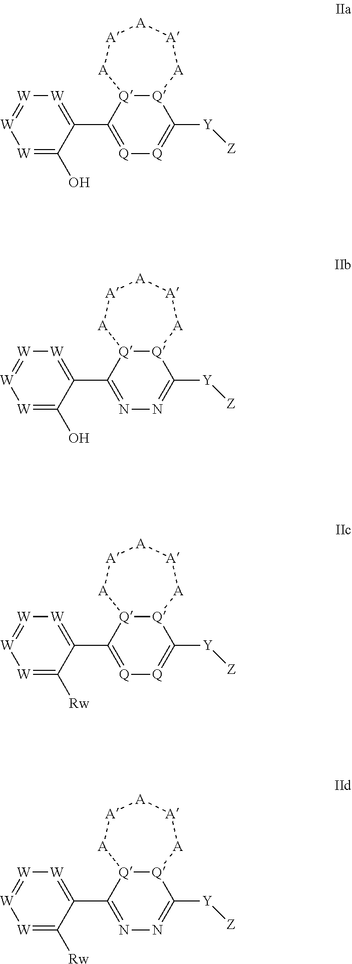

wherein:
R_{w} is hydrogen, C₁₋₆alkyl, C₂₋₈alkynyl, halogen, C₁₋₆alkoxy, halo-C₁₋₄alkyl, halo-C₁₋₄alkoxy, cyano, —NH₂, —N(C₁₋₆alkyl)₂, thiol, —SO₂NH₂, —SO₂N(C₁₋₆alkyl)₂, —S(═O)(C₁₋₆alkyl), cycloalkyl, CHR₁ₐ, (C═O)R₁ₐ, OR₁ₐ, N(R_{1b})₂, S(═O)R₁ₐ, S(═O)₂R₁ₐ, O(C═O)R₁ₐ, (C═O)OR₁ₐ, NR_{1b}(C═O)R_{1b}, (C═O)NHR_{1b}, (C═O)N(R_{1b})₂ NR_{1b}(C═O)OR₁ₐ, O(C═O)N(R_{1b})₂, ONR_{1b}(C═NR_{1b})NR_{1b}, NR_{1b}S(═O)₂R₁ₐ or S(═O)₂N(R_{1b})₂, wherein each C₁₋₆alkyl, heterocycle, heteroaryl and aryl are optionally substituted with 1 or 2 substituents each selected from R₅; each W is independently CH, CR′ or N, wherein at least one Q′ is N;
each Q is independently N or CH;
each Q′ is independently N, C or CH;
each A is independently CH, CH₂, CRₐ, CHRₐ, CR₄, CHR₄, N, NH, NRₐ, NR₄, S, or O; each A′ is independently absent, CH, CH₂, CRₐ, CHRₐ, CR₄, CHR₄, N, NH, NRₐ, NR₄, S, or O;
each Rₐ is independently H, deuterium, halogen, —CN, —OH, —OR₂, ═O, ═N—OR₂, —SR₂, —S(═O)R₂, —S(═O)₂R², —N(R₂)₂, —NR₂S(═O)(═NR₂)R², —NR₂S(═O)₂R², —S(═O)₂N(R₂)₂, —C(═O)R₂, —OC(═O)R₂, —C(═O)OR₂, —OC(═O)OR₂, —C(═O)N(R₂)₂, —OC(═O)N(R₂)₂, —NR₂C(═O)R₂, —P(═O)(R₂)₂, C₁₋₄alkyl, (C₁₋₄alkyl)₂, halo-C₁₋₆alkyl, C₁₋₆heteroalkyl, C₃₋₈cycloalkyl, C₂₋₇heterocycloalkyl, aryl or monocyclic heteroaryl;
each — represents either an absent, single, or double bond;
Y is C(R₁ₐ)₂, C═O, O, NR_{1b} or a bond;
each R₁ₐ is independently hydrogen, halogen, hydroxyl, cyano, C₁₋₄alkyl, deutero-C₁₋₄alkyl, halo-C₁₋₄alkyl, amino or hydroxy-C₁₋₄alkyl, C₃₋₁₀cycloalkyl, C₂₋₇heterocycloalkyl or aryl;
each R_{1b} is independently hydrogen, C₁₋₄alkyl, deutero-C₁₋₄alkyl, halo-C₁₋₄alkyl or hydroxy-C₁₋₄alkyl;
each R′ is independently heterocyclyl, heteroaryl, aryl, C₃₋₈cycloalkyl, C₁₋₄alkyl, C₁₋₄alkoxy, deutero-C₁₋₄alkyl, halo-C₁₋₄alkyl, halo-C₁₋₄alkoxy, hydroxy-C₁₋₄alkyl, halogen, hydroxy or cyano,
wherein each heterocycle, heteroaryl, C₃₋₈cycloalkyl and aryl are optionally substituted with 1, or 2, substituents each selected from R₃;
each Z is heterocyclyl, heteroaryl, aryl, C₃₋₁₀cycloalkyl, C₁₋₄alkyl, deutero-C₁₋₄alkyl, halo-C₁₋₄alkyl, hydroxy-C₁₋₈alkyl, C₁₋₆alkoxy, NH(hydroxyl-C₁₋₆alkyl), NH(C₁₋₆alkoxy) wherein each Z is optionally substituted with OH, NH₂, —CO₂H, halogen, C₁₋₆alkyl, C₁₋₆haloalkyl, C₁₋₆ hydroxyalkyl, C₂₋₆acyl, C₂₋₆alkanoic acid, C₂₋₆alkanoate ester, or heterocyclyl, and wherein heterocyclyl, C₃₋₁₀cycloalkyl a saturated or partially unsaturated 3-7 membered monocyclic, 6-10 membered bicyclic or 13-16 membered polycyclic ring system having 1, 2, or 3 heteroatom ring members independently selected from N, O, and S, each optionally substituted with 1, 2, 3, 4, or 5 substituents each selected from R₂;
R₂ is independently selected from halogen, hydroxyl, cyano, C₁₋₄alkyl, hydroxy-C₁₋₄alkyl, deutero-C₁₋₄alkyl, halo-C₁₋₄alkyl, amino, C₁₋₄alkyl-amino, (C₁₋₆alkyl)₂-amino, halo-C₁₋₄alkyl-amino, (halo-C₁₋₆alkyl)₂-amino, hydroxy-C₁₋₄alkyl-amino, C₁₋₄alkoxy-C₁₋₄alkyl-amino, amino-C₁₋₄alkyl, C₁₋₄alkyl-amino-C₁₋₄alkyl, (C₁₋₄alkyl-amino)₂-C₁₋₄alkyl, C₁₋₄alkoxy, halo-C₁₋₄alkoxy, hydroxy-C₁₋₄alkoxy, C₁₋₄alkyl-C₁₋₄alkoxy, C₃₋₁₀cycloalkyl, C₃₋₁₀cycloalkyl-amino, C₃₋₁₀cycloalkyl-amino-C₁₋₄alkyl, heteroaryl-C₁₋₄alkyl, heteroaryl-amino, heteroaryl-C₁₋₄alkyl-amino, heterocyclyl, heterocyclyl-C₁₋₄alkyl, heterocyclyl-amino, heterocyclyl-amino-C₁₋₄alkyl, heterocyclyl-C₁₋₄alkoxy, heterocyclyl-amino-C₃₋₁₀cycloalkyl, phenyl, and phenyl-C₁₋₄alkoxy,
wherein heteroaryl is a 5-6 membered monocyclic or 6-10 membered bicyclic ring system having 1, 2, 3, or 4 heteroatom ring members independently selected from N, O, and S,
wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S,
wherein C₃₋₁₀cycloalkyl is a saturated or partially unsaturated 3-7 membered monocyclic ring system, and
wherein each instance of phenyl, heteroaryl, heterocyclyl, or C₃₋₁₀cycloalkyl is optionally substituted with 1 or 2 substituents each selected from R₃;
R₃ is independently selected from halogen, hydroxyl, cyano, C₁₋₄alkyl, deutero-C₁₋₄alkyl, halo-C₁₋₄alkyl, amino, C₁₋₄alkoxy, and halo-C₁₋₄alkoxy;
each R′ and R₄ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, halo-C₁₋₄alkoxy, heteroaryl, heterocyclyl, and phenyl,
wherein heteroaryl is a 5-6 membered monocyclic or 9-10 membered bicyclic ring system having 1, 2, 3, or 4 heteroatom ring members selected from N, O, and S,
wherein heterocyclyl is a saturated or partially unsaturated₃₋₆ membered monocyclic or 9-10 membered bicyclic ring system having 1, 2, or 3 heteroatom ring members selected from N, O, and S, and
wherein each instance of phenyl, heteroaryl or heterocyclyl is optionally substituted with 1, or 2 substituents each selected from R₅;
R₅ is independently selected from halogen, hydroxyl, cyano, nitro, C₁₋₄alkyl, deutero-C₁₋₄alkyl, halo-C₁₋₄alkyl, amino, C₁₋₄alkyl-amino, (C₁₋₄alkyl)₂-amino, amino-C₁₋₄alkyl, hydroxy-C₁₋₄alkyl, C₁₋₄alkyl-carbonyl, C₁₋₄alkoxy, C₁₋₄alkylthio, halo-C₁₋₄alkoxy, and C₃₋₁₀cycloalkyl; and wherein a form of the compound is selected from the group consisting of a pharmaceutically acceptable salt, hydrate, solvate, racemate, enantiomer, diastereomer, stereoisomer, tautomer, and isotope enriched form thereof.

Another aspect of the invention provides a compound having the structure of Formulae IIIa, IIIb, IIIc, or IIId:

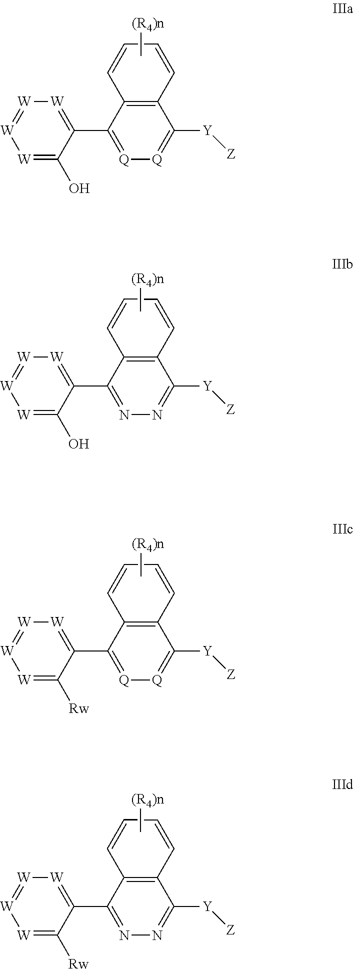

wherein:
R_{w} is hydrogen, C₁₋₆alkyl, C₂₋₈alkynyl, halogen, C₁₋₆alkoxy, halo-C₁₋₄alkyl, halo-C₁₋₄alkoxy, cyano, —NH₂, —N(C₁₋₆alkyl)₂, thiol, —SO₂NH₂, —SO₂N(C₁₋₆alkyl)₂, —S(═O)(C₁₋₆alkyl), cycloalkyl, CHR₁ₐ, (C═O)R₁ₐ, OR₁ₐ, N(R_{1b})₂, S(═O)R₁ₐ, S(═O)₂R₁ₐ, O(C═O)R₁ₐ, (C═O)OR₁ₐ, NR_{1b}(C═O)R_{1b}, (C═O)NHR_{1b}, (C═O)N(R_{1b})₂ NR_{1b}(C═O)OR₁ₐ, O(C═O)N(R_{1b})₂, ONR_{1b}(C═NR_{1b})NR_{1b}, NR_{1b}S(═O)₂R₁ₐ or S(═O)₂N(R_{1b})₂, wherein each heterocycle, heteroaryl and aryl are optionally substituted with 1 or 2 substituents each selected from R₅;
each W is independently CH, CR′ or N;
each Q is independently N, or CH;
Y is C(R₁ₐ)₂, C═O, O, NR_{1b} or a bond;
each R₁ₐ is independently hydrogen, halogen, hydroxyl, cyano, C₁₋₄alkyl, deutero-C₁₋₄alkyl, halo-C₁₋₄alkyl, amino or hydroxy-C₁₋₄alkyl;
each R_{1b} is independently hydrogen, C₁₋₄alkyl, deutero-C₁₋₄alkyl, halo-C₁₋₄alkyl or hydroxy-C₁₋₄alkyl;
each R₄ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, halo-C₁₋₄alkoxy, heteroaryl, heterocyclyl, and phenyl,
wherein heteroaryl is a 5-6 membered monocyclic or 9-10 membered bicyclic ring system having 1, 2, 3, or 4 heteroatom ring members selected from N, O, and S,
wherein heterocyclyl is a saturated or partially unsaturated 3-6 membered monocyclic or 9-10 membered bicyclic ring system having 1, 2, or 3 heteroatom ring members selected from N, O, and S, and
wherein each instance of phenyl, heteroaryl or heterocyclyl is optionally substituted with 1, or 2 substituents each selected from R₅;
each Z is heterocyclyl, heteroaryl, aryl, C₃₋₁₀cycloalkyl, C₁₋₄alkyl, deutero-C₁₋₄alkyl, halo-C₁₋₄alkyl, hydroxy-C₁₋₈alkyl, NH(hydroxy-C₁₋₆alkyl), NH(C₁₋₆alkoxy) wherein each Z is optionally substituted with OH, NH₂, —CO₂H, halogen, C₁₋₆alkyl, C₁₋₆haloalkyl, C₁₋₆ hydroxyalkyl, C₂₋₆acyl, C₂₋₆alkanoic acid, C₂₋₆alkanoate ester, or heterocyclyl, wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S, each optionally substituted with 1, 2, 3, 4, or 5 substituents each selected from R₂;
R₂ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₆alkyl)₂-amino, halo-C₁₋₄alkyl-amino, (halo-C₁₋₆alkyl)₂-amino, hydroxy-C₁₋₄alkyl-amino, C₁₋₄alkoxy-C₁₋₄alkyl-amino, amino-C₁₋₄alkyl, C₁₋₄alkyl-amino-C₁₋₄alkyl, (C₁₋₄alkyl-amino)₂-C₁₋₄alkyl, C₁₋₄alkoxy, halo-C₁₋₄alkoxy, hydroxy-C₁₋₄alkoxy, C₁₋₄alkyl-C₁₋₄alkoxy, C₃₋₁₀cycloalkyl, C₃₋₁₀cycloalkyl-amino, C₃₋₁₀cycloalkyl-amino-C₁₋₄alkyl, heteroaryl-C₁₋₄alkyl, heteroaryl-amino, heteroaryl-C₁₋₄alkyl-amino, heterocyclyl, heterocyclyl-C₁₋₄alkyl, heterocyclyl-amino, heterocyclyl-amino-C₁₋₄alkyl, heterocyclyl-C₁₋₄alkoxy, heterocyclyl-amino-C₃₋₁₀cycloalkyl, phenyl, and phenyl-C₁₋₄alkoxy,
wherein heteroaryl is a 5-6 membered monocyclic or 6-10 membered bicyclic ring system having 1, 2, 3, or 4 heteroatom ring members independently selected from N, O, and S,
wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S,
wherein C₃₋₁₀cycloalkyl is a saturated or partially unsaturated 3-7 membered monocyclic ring system, and
wherein each instance of phenyl, heteroaryl, heterocyclyl, or C₃₋₁₀cycloalkyl is optionally substituted with 1 or 2 substituents each selected from R₃;
R₃ is independently selected from halogen, hydroxyl, cyano, C₁₋₄alkyl, deutero-C₁₋₄alkyl, halo-C₁₋₄alkyl, amino, C₁₋₄alkoxy, and halo-C₁₋₄alkoxy;
each R′ and R₄ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, halo-C₁₋₄alkoxy, heteroaryl, heterocyclyl, and phenyl,
wherein heteroaryl is a 5-6 membered monocyclic or 9-10 membered bicyclic ring system having 1, 2, 3, or 4 heteroatom ring members selected from N, O, and S,
wherein heterocyclyl is a saturated or partially unsaturated 3-6 membered monocyclic or 9-10 membered bicyclic ring system having 1, 2, or 3 heteroatom ring members selected from N, O, and S,
wherein each instance of phenyl, heteroaryl or heterocyclyl is optionally substituted with 1, or 2 substituents each selected from R₅;
R₅ is independently selected from halogen, hydroxyl, cyano, nitro, C₁₋₄alkyl, deutero-C₁₋₄alkyl, halo-C₁₋₄alkyl, amino, C₁₋₄alkyl-amino, (C₁₋₄alkyl)₂-amino, amino-C₁₋₄alkyl, hydroxy-C₁₋₄alkyl, C₁₋₄alkyl-carbonyl, C₁₋₄alkoxy, C₁₋₄alkylthio, halo-C₁₋₄alkoxy, and C₃₋₁₀cycloalkyl; and
n is 0, 1, 2, or 3;
wherein a form of the compound is selected from the group consisting of a pharmaceutically acceptable salt, hydrate, solvate, racemate, enantiomer, diastereomer, stereoisomer, tautomer, and isotope enriched form thereof.

Another aspect of the invention provides a compound having the structure of Formulae IVa, IVb, IVc, or IVd:

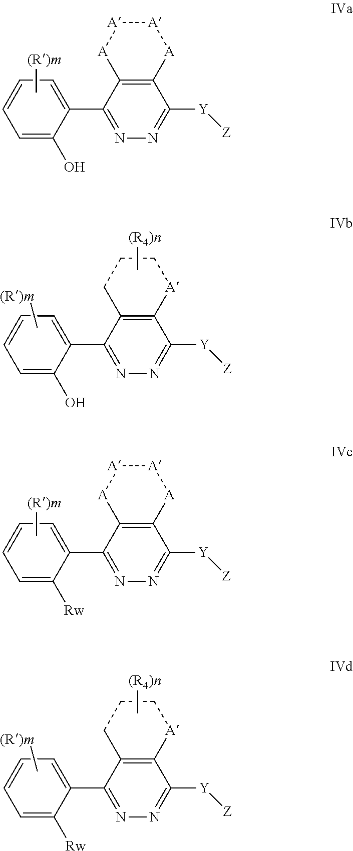

wherein:
R_{w} is hydrogen, C₁₋₆alkyl, C₂₋₈alkynyl, halogen, C₁₋₆alkoxy, halo-C₁₋₄alkyl, halo-C₁₋₄alkoxy, cyano, —NH₂, —N(C₁₋₆alkyl)₂, thiol, —SO₂NH₂, —SO₂N(C₁₋₆alkyl)₂, —S(═O)(C₁₋₆alkyl), cycloalkyl, CHR₁ₐ, (C═O)R₁ₐ, OR₁ₐ, N(R_{1b})₂, S(═O)R₁ₐ, S(═O)₂R₁ₐ, O(C═O)R₁ₐ, (C═O)OR₁ₐ, NR_{1b}(C═O)R_{1b}, (C═O)NHR_{1b}, (C═O)N(R_{1b})₂ NR_{1b}(C═O)OR₁ₐ, O(C═O)N(R_{1b})₂, ONR_{1b}(C═NR_{1b})NR_{1b}, NR_{1b}S(═O)₂R₁ₐ or S(═O)₂N(R_{1b})₂, wherein each heterocycle, heteroaryl and aryl are optionally substituted with 1 or 2 substituents each selected from R₅ each A is independently NH, S, O, CH₂, or CR₄;
A′ is independently NH, S, O, CH₂, CR₄ or absent;
Each [symbol] represents either an absent, single, or double bond;
Y is C(R₁ₐ)₂, C═O, O, NR_{1b} or a bond;
R₁ₐ is hydrogen, halogen, hydroxyl, cyano, C₁₋₄alkyl, deutero-C₁₋₄alkyl, halo-C₁₋₄alkyl, amino or hydroxy-C₁₋₄alkyl;
R_{1b} is hydrogen, C₁₋₄alkyl, deutero-C₁₋₄alkyl, halo-C₁₋₄alkyl or hydroxy-C₁₋₄alkyl; and each Z is heterocyclyl, heteroaryl, aryl, C₃₋₁₀cycloalkyl, C₁₋₄alkyl, deutero-C₁₋₄alkyl, halo-C₁₋₄alkyl, hydroxy-C₁₋₈alkyl, NH(hydroxy-C₁₋₆alkyl), NH(C₁₋₆alkoxy) wherein each Z is optionally substituted with OH, NH₂, —CO₂H, halogen, C₁₋₆alkyl, C₁₋₆haloalkyl, C₁₋₆ hydroxyalkyl, C₂₋₆acyl, C₂₋₆alkanoic acid, C₂₋₆alkanoate ester, or heterocyclyl, wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S, each optionally substituted with 1, 2, 3, 4, or 5 substituents each selected from R₂;
R₂ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₆alkyl)₂-amino, halo-C₁₋₄alkyl-amino, (halo-C₁₋₆alkyl)₂-amino, hydroxy-C₁₋₄alkyl-amino, C₁₋₄alkoxy-C₁₋₄alkyl-amino, amino-C₁₋₄alkyl, C₁₋₄alkyl-amino-C₁₋₄alkyl, (C₁₋₄alkyl-amino)₂-C₁₋₄alkyl, C₁₋₄alkoxy, halo-C₁₋₄alkoxy, hydroxy-C₁₋₄alkoxy, C₁₋₄alkyl-C₁₋₄alkoxy, C₃₋₁₀cycloalkyl, C₃₋₁₀cycloalkyl-amino, C₃₋₁₀cycloalkyl-amino-C₁₋₄alkyl, heteroaryl-C₁₋₄alkyl, heteroaryl-amino, heteroaryl-C₁₋₄alkyl-amino, heterocyclyl, heterocyclyl-C₁₋₄alkyl, heterocyclyl-amino, heterocyclyl-amino-C₁₋₄alkyl, heterocyclyl-C₁₋₄alkoxy, heterocyclyl-amino-C₃₋₁₀cycloalkyl, phenyl, and phenyl-C₁₋₄alkoxy,
wherein heteroaryl is a 5-6 membered monocyclic or 6-10 membered bicyclic ring system having 1, 2, 3, or 4 heteroatom ring members independently selected from N, O, and S,
wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S,
wherein C₃₋₁₀cycloalkyl is a saturated or partially unsaturated 3-7 membered monocyclic ring system, and
wherein each instance of phenyl, heteroaryl, heterocyclyl, or C₃₋₁₀cycloalkyl is optionally substituted with 1 or 2 substituents each selected from R₃;
R₃ is independently selected from halogen, hydroxyl, cyano, C₁₋₄alkyl, deutero-C₁₋₄alkyl, halo-C₁₋₄alkyl, amino, C₁₋₄alkoxy, and halo-C₁₋₄alkoxy;
each R₄ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, halo-C₁₋₄alkoxy, heteroaryl, heterocycle, and phenyl,
wherein heteroaryl is a 5-6 membered monocyclic or 9-10 membered bicyclic ring system having 1, 2, 3, or 4 heteroatom ring members selected from N, O, and S,
wherein heterocyclyl is a saturated or partially unsaturated 3-6 membered monocyclic or 9-10 membered bicyclic ring system having 1, 2, or 3 heteroatom ring members selected from N, O, and S, and
wherein each instance of phenyl, heteroaryl or heterocyclyl is optionally substituted with 1, or 2 substituents each selected from R₅;
R₅ is independently selected from halogen, hydroxyl, cyano, nitro, C₁₋₄alkyl, deutero-C₁₋₄alkyl, halo-C₁₋₄alkyl, amino, C₁₋₄alkyl-amino, (C₁₋₄alkyl)₂-amino, amino-C₁₋₄alkyl, hydroxy-C₁₋₄alkyl, C₁₋₄alkyl-carbonyl, C₁₋₄alkoxy, C₁₋₄alkylthio, halo-C₁₋₄alkoxy, and C₃₋₁₀cycloalkyl;
m is 0, 1, 2, 3, or 4; and
n is 0, 1, 2, or 3;
wherein a form of the compound is selected from the group consisting of a pharmaceutically acceptable salt, hydrate, solvate, racemate, enantiomer, diastereomer, stereoisomer, tautomer, and isotope enriched form thereof.

Another aspect of the invention provides a compound having the structure of Formulae Va, Vb, Vc, or Vd:

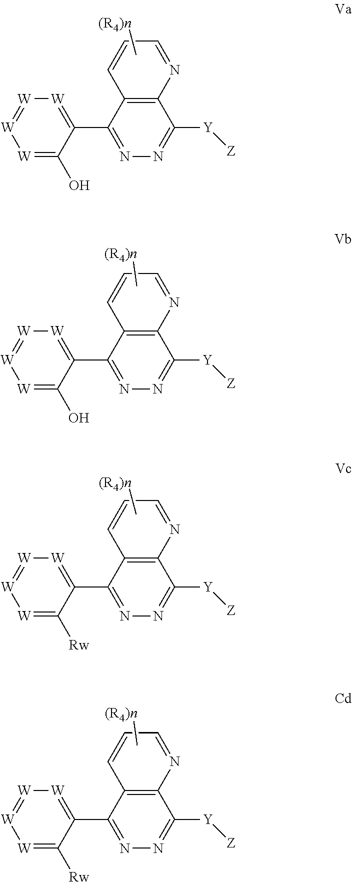

wherein:
R_{w} is hydrogen, C₁₋₆alkyl, C₂₋₈alkynyl, halogen, C₁₋₆alkoxy, halo-C₁₋₄alkyl, halo-C₁₋₄ alkoxy, cyano, —NH₂, —N(C₁₋₆alkyl)₂, thiol, —SO₂NH₂, —SO₂N(C₁₋₆alkyl)₂, —S(═O)(C₁₋₆alkyl), cycloalkyl, CHR₁ₐ, (C═O)R₁ₐ, OR₁ₐ, N(R_{1b})₂, S(═O)R₁ₐ, S(═O)₂R₁ₐ, O(C═O)R₁ₐ, (C═O)OR₁ₐ, NR_{1b}(C═O)R_{1b}, (C═O)NHR_{1b}, (C═O)N(R_{1b})₂ NR_{1b}(C═O)OR₁ₐ, O(C═O)N(R_{1b})₂, ONR_{1b}(C═NR_{1b})NR_{1b}, NR_{1b}S(═O)₂R₁ₐ or S(═O)₂N(R_{1b})₂, wherein each heterocycle, heteroaryl and aryl are optionally substituted with 1 or 2 substituents each selected from R₅;
each W is independently CH, CR′ or N;
Y is C(R₁ₐ)₂, C═O, O, NR_{1b} or a bond;
R₁ₐ is hydrogen, halogen, hydroxyl, cyano, C₁₋₄alkyl, deutero-C₁₋₄alkyl, halo-C₁₋₄alkyl, amino or hydroxy-C₁₋₄alkyl;
R_{1b} is hydrogen, C₁₋₄alkyl, deutero-C₁₋₄alkyl, halo-C₁₋₄alkyl or hydroxy-C₁₋₄alkyl; and
each Z is heterocyclyl, heteroaryl, aryl, C₃₋₁₀cycloalkyl, C₁₋₄alkyl, deutero-C₁₋₄alkyl, halo-C₁₋₄alkyl, hydroxy-C₁₋₈alkyl, NH(hydroxy-C₁₋₆alkyl), NH(C₁₋₆alkoxy) wherein each Z is optionally substituted with OH, NH₂, —CO₂H, halogen, C₁₋₆alkyl, C₁₋₆haloalkyl, C₁₋₆ hydroxyalkyl, C₂₋₆acyl, C₂₋₆alkanoic acid, C₂₋₆alkanoate ester, or heterocyclyl, wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S, each optionally substituted with 1, 2, 3, 4, or 5 substituents each selected from R₂;
R₂ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₆alkyl)₂-amino, halo-C₁₋₄alkyl-amino, (halo-C₁₋₆alkyl)₂-amino, hydroxy-C₁₋₄alkyl-amino, C₁₋₄alkoxy-C₁₋₄alkyl-amino, amino-C₁₋₄alkyl, C₁₋₄alkyl-amino-C₁₋₄alkyl, (C₁₋₄alkyl-amino)₂-C₁₋₄alkyl, C₁₋₄alkoxy, halo-C₁₋₄alkoxy, hydroxy-C₁₋₄alkoxy, C₁₋₄alkyl-C₁₋₄alkoxy, C₃₋₁₀cycloalkyl, C₃₋₁₀cycloalkyl-amino, C₃₋₁₀cycloalkyl-amino-C₁₋₄alkyl, heteroaryl-C₁₋₄alkyl, heteroaryl-amino, heteroaryl-C₁₋₄alkyl-amino, heterocyclyl, heterocyclyl-C₁₋₄alkyl, heterocyclyl-amino, heterocyclyl-amino-C₁₋₄alkyl, heterocyclyl-C₁₋₄alkoxy, heterocyclyl-amino-C₃₋₁₀cycloalkyl, phenyl, and phenyl-C₁₋₄alkoxy,
wherein heteroaryl is a 5-6 membered monocyclic or 6-10 membered bicyclic ring system having 1, 2, 3, or 4 heteroatom ring members independently selected from N, O, or S,
wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S,
wherein C₃₋₁₀cycloalkyl is a saturated or partially unsaturated 3-7 membered monocyclic ring system,
wherein each instance of phenyl, heteroaryl, heterocyclyl, or C₃₋₁₀cycloalkyl is optionally substituted with 1 or 2 substituents each selected from R₃;
R₃ is independently selected from halogen, hydroxyl, cyano, C₁₋₄alkyl, deutero-C₁₋₄alkyl, halo-C₁₋₄alkyl, amino, C₁₋₄alkoxy, and halo-C₁₋₄alkoxy;
each R′ or R₄ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, halo-C₁₋₄alkoxy, heteroaryl, heterocyclyl, and phenyl,
wherein heteroaryl is a 5-6 membered monocyclic or 9-10 membered bicyclic ring system having 1, 2, 3, or 4 heteroatom ring members selected from N, O, and S,
wherein heterocyclyl is a saturated or partially unsaturated 3-6 membered monocyclic or 9-10 membered bicyclic ring system having 1, 2, or 3 heteroatom ring members selected from N, O, and S, and
wherein each instance of phenyl, heteroaryl or heterocyclyl is optionally substituted with 1, or 2 substituents each selected from R₅;
R₅ is independently selected from halogen, hydroxyl, cyano, nitro, C₁₋₄alkyl, deutero-C₁₋₄alkyl, halo-C₁₋₄alkyl, amino, C₁₋₄alkyl-amino, (C₁₋₄alkyl)₂-amino, amino-C₁₋₄alkyl, hydroxy-C₁₋₄alkyl, C₁₋₄alkyl-carbonyl, C₁₋₄alkoxy, C₁₋₄alkylthio, halo-C₁₋₄alkoxy, and C₃₋₁₀cycloalkyl; and
n is 0, 1, 2, or 3;
wherein a form of the compound is selected from the group consisting of a pharmaceutically acceptable salt, hydrate, solvate, racemate, enantiomer, diastereomer, stereoisomer, tautomer, and isotope enriched form thereof.

Another aspect of the invention provides a compound having the structure of Formulae VIa, VIb, VIc, or VId:

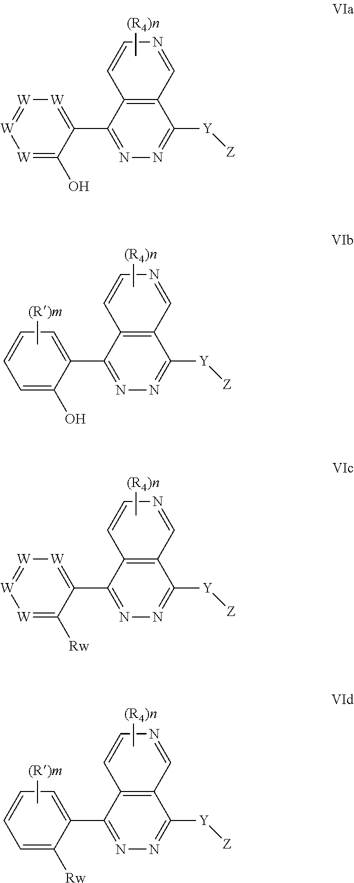

wherein:
R_{w} is hydrogen, C₁₋₆alkyl, C₂₋₈alkynyl, halogen, C₁₋₆alkoxy, halo-C₁₋₄alkyl, halo-C₁₋₄alkoxy, cyano, —NH₂, —N(C₁₋₆alkyl)₂, thiol, —SO₂NH₂, —SO₂N(C₁₋₆alkyl)₂, —S(═O)(C₁₋₆alkyl), cycloalkyl, CHR₁ₐ, (C═O)R₁ₐ, OR₁ₐ, N(R_{1b})₂, S(═O)R₁ₐ, S(═O)₂R₁ₐ, O(C═O)R₁ₐ, (C═O)OR₁ₐ, NR_{1b}(C═O)R_{1b}, (C═O)NHR_{1b}, (C═O)N(R_{1b})₂ NR_{1b}(C═O)OR₁ₐ, O(C═O)NHR_{1b}, ONR_{1b}(C═NR_{1b})NR_{1b}, NR_{1b}S(═O)₂R₁ₐ or S(═O)₂N(R_{1b})₂, wherein each heterocycle, heteroaryl and aryl are optionally substituted with 1 or 2 substituents each selected from R₅;
each W is independently CH, CR′ or N;
Y is C(R₁ₐ)₂, C═O, O, NR_{1b} or a bond
each R₁ₐ is independently hydrogen, halogen, hydroxyl, cyano, C₁₋₄alkyl, deutero-C₁₋₄alkyl, halo-C₁₋₄alkyl, amino or hydroxy-C₁₋₄alkyl;
each R_{1b} is independently hydrogen, C₁₋₄alkyl, deutero-C₁₋₄alkyl, halo-C₁₋₄alkyl or hydroxy-C₁₋₄alkyl;
each Z is heterocyclyl, heteroaryl, aryl, C₃₋₁₀cycloalkyl, C₁₋₄alkyl, deutero-C₁₋₄alkyl, halo-C₁₋₄alkyl, hydroxy-C₁₋₈alkyl, NH(hydroxy-C₁₋₆alkyl), NH(C₁₋₆alkoxy) wherein each Z is optionally substituted with OH, NH₂, —CO₂H, halogen, C₁₋₆alkyl, C₁₋₆haloalkyl, C₁₋₆ hydroxyalkyl, C₂₋₆acyl, C₂₋₆alkanoic acid, C₂₋₆alkanoate ester, or heterocyclyl, wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S, each optionally substituted with 1, 2, 3, 4, or 5 substituents each selected from R₂;
R₂ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₆alkyl)₂-amino, halo-C₁₋₄alkyl-amino, (halo-C₁₋₆alkyl)₂-amino, hydroxy-C₁₋₄alkyl-amino, C₁₋₄alkoxy-C₁₋₄alkyl-amino, amino-C₁₋₄alkyl, C₁₋₄alkyl-amino-C₁₋₄alkyl, (C₁₋₄alkyl-amino)₂-C₁₋₄alkyl, C₁₋₄alkoxy, halo-C₁₋₄alkoxy, hydroxy-C₁₋₄alkoxy, C₁₋₄alkyl-C₁₋₄alkoxy, C₃₋₁₀cycloalkyl, C₃₋₁₀cycloalkyl-amino, C₃₋₁₀cycloalkyl-amino-C₁₋₄alkyl, heteroaryl-C₁₋₄alkyl, heteroaryl-amino, heteroaryl-C₁₋₄alkyl-amino, heterocyclyl, heterocyclyl-C₁₋₄alkyl, heterocyclyl-amino, heterocyclyl-amino-C₁₋₄alkyl, heterocyclyl-C₁₋₄alkoxy, heterocyclyl-amino-C₃₋₁₀cycloalkyl, phenyl, and phenyl-C₁₋₄alkoxy,
wherein heteroaryl is a 5-6 membered monocyclic or 6-10 membered bicyclic ring system having 1, 2, 3, or 4 heteroatom ring members independently selected from N, O, and S,
wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S,
wherein C₃₋₁₀cycloalkyl is a saturated or partially unsaturated 3-7 membered monocyclic ring system, and
wherein each instance of phenyl, heteroaryl, heterocyclyl, or C₃₋₁₀cycloalkyl is optionally substituted with 1 or 2 substituents each selected from R₃;
R₃ is independently selected from halogen, hydroxyl, cyano, C₁₋₄alkyl, deutero-C₁₋₄alkyl, halo-C₁₋₄alkyl, amino, C₁₋₄alkoxy, and halo-C₁₋₄alkoxy;
each R′ or R₄ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, halo-C₁₋₄alkoxy, heteroaryl, heterocyclyl, and phenyl,
wherein heteroaryl is a 5-6 membered monocyclic or 9-10 membered bicyclic ring system having 1, 2, 3, or 4 heteroatom ring members selected from N, O, and S,
wherein heterocyclyl is a saturated or partially unsaturated 3-6 membered monocyclic or 9-10 membered bicyclic ring system having 1, 2, or 3 heteroatom ring members selected from N, O, and S, and
wherein each instance of phenyl, heteroaryl or heterocyclyl is optionally substituted with 1, or 2 substituents each selected from R₅;
R₅ is independently selected from halogen, hydroxyl, cyano, nitro, C₁₋₄alkyl, deutero-C₁₋₄alkyl, halo-C₁₋₄alkyl, amino, C₁₋₄alkyl-amino, (C₁₋₄alkyl)₂-amino, amino-C₁₋₄alkyl, hydroxy-C₁₋₄alkyl, C₁₋₄alkyl-carbonyl, C₁₋₄alkoxy, C₁₋₄alkylthio, halo-C₁₋₄alkoxy, and C₃₋₁₀cycloalkyl; and n is 0, 1, 2, or 3;
wherein a form of the compound is selected from the group consisting of a pharmaceutically acceptable salt, hydrate, solvate, racemate, enantiomer, diastereomer, stereoisomer, tautomer, and isotope enriched form thereof.

Another aspect of the invention provides a compound having the structure of Formulae VIIa, VIIb, VIIc, or VIId:

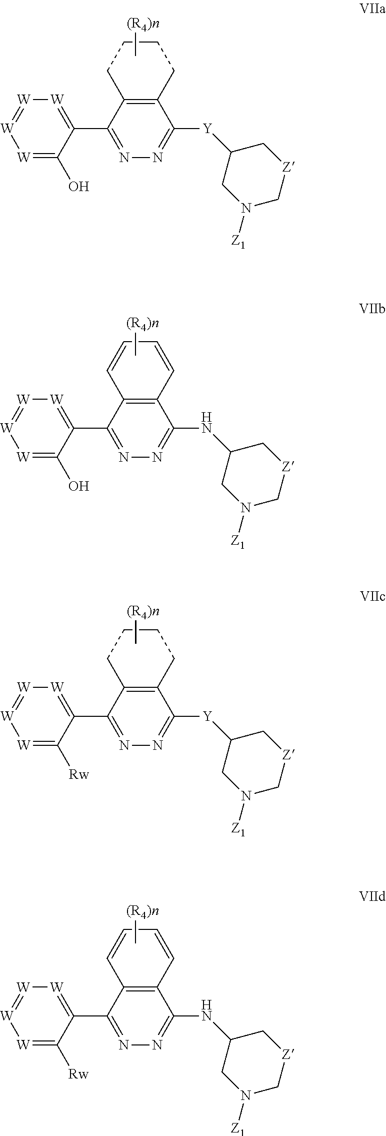

wherein:
R_{w} is hydrogen, C₁₋₆alkyl, C₂₋₈alkynyl, halogen, C₁₋₆alkoxy, halo-C₁₋₄alkyl, halo-C₁₋₄alkoxy, cyano, —NH₂, —N(C₁₋₆alkyl)₂, thiol, —SO₂NH₂, —SO₂N(C₁₋₆alkyl)₂, —S(═O)(C₁₋₆alkyl), cycloalkyl, CHR₁ₐ, (C═O)R₁ₐ, OR₁ₐ, N(R_{1b})₂, S(═O)R₁ₐ, S(═O)₂R₁ₐ, O(C═O)R₁ₐ, (C═O)OR₁ₐ, NR_{1b}(C═O)R_{1b}, (C═O)NHR_{1b}, (C═O)N(R_{1b})₂ NR_{1b}(C═O)OR₁ₐ, O(C═O)N(R_{1b})₂, ONR_{1b}(C═NR_{1b})NR_{1b}, NR_{1b}S(═O)₂R₁ₐ or S(═O)₂N(R_{1b})₂, wherein each heterocycle, heteroaryl and aryl are optionally substituted with 1 or 2 substituents each selected from R₅;
each W is independently CH, CR′ or N;
each [symbol] represents either an absent, single, or double bond;
each Z′ is independently CH₂ or absent;
Y is C(R₁ₐ)₂, C═O, O, NR_{1b} or a bond;
each R₁ₐ is independently hydrogen, halogen, hydroxyl, cyano, C₁₋₄alkyl, deutero-C₁₋₄alkyl, halo-C₁₋₄alkyl, amino or hydroxy-C₁₋₄alkyl;
each R_{1b} is independently hydrogen, C₁₋₄alkyl, deutero-C₁₋₄alkyl, halo-C₁₋₄alkyl or hydroxy-C₁₋₄alkyl; and
each Z₁, R′, and R₄ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, halo-C₁₋₄alkoxy, heteroaryl, heterocyclyl, and phenyl,
wherein heteroaryl is a 5-6 membered monocyclic or 9-10 membered bicyclic ring system having 1, 2, 3, or 4 heteroatom ring members selected from N, O, and S,
wherein heterocyclyl is a saturated or partially unsaturated 3-6 membered monocyclic or 9-10 membered bicyclic ring system having 1, 2, or 3 heteroatom ring members selected from N, O, and S, and
wherein each instance of phenyl, heteroaryl or heterocyclyl is optionally substituted with 1, or 2 substituents each selected from R₅;
R₅ is independently selected from halogen, hydroxyl, cyano, nitro, C₁₋₄alkyl, deutero-C₁₋₄alkyl, halo-C₁₋₄alkyl, amino, C₁₋₄alkyl-amino, (C₁₋₄alkyl)₂-amino, amino-C₁₋₄alkyl, hydroxy-C₁₋₄alkyl, C₁₋₄alkyl-carbonyl, C₁₋₄alkoxy, C₁₋₄alkylthio, halo-C₁₋₄alkoxy, and C₃₋₁₀cycloalkyl; and
n is 0, 1, 2, or 3;
wherein a form of the compound is selected from the group consisting of a pharmaceutically acceptable salt, hydrate, solvate, racemate, enantiomer, diastereomer, stereoisomer, tautomer, and isotope enriched form thereof.

Another aspect of the invention provides a compound having the structure of Formulae VIIIa, VIIIb, VIIIc, VIIId, VIIIe, or VIIIf:

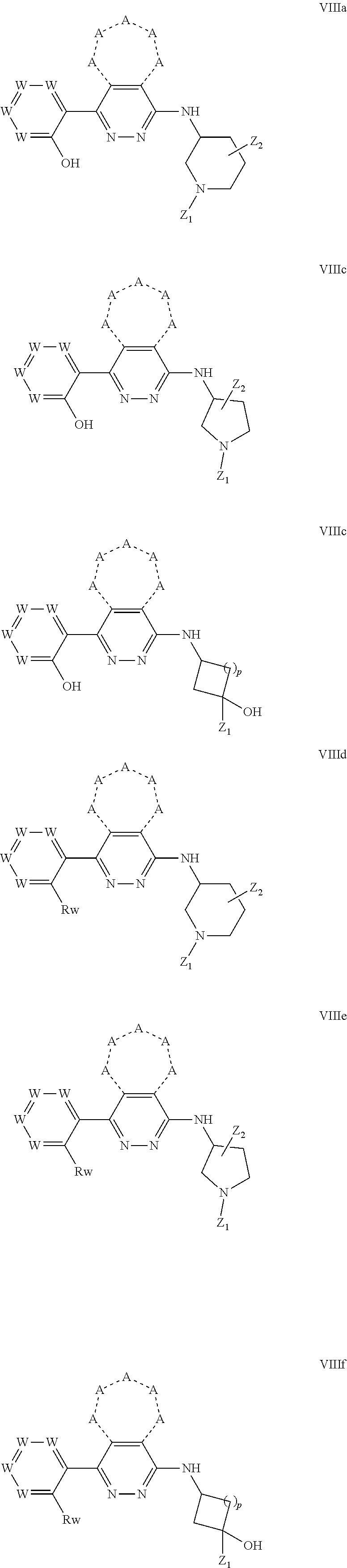

R_{w} is hydrogen, C₁₋₆alkyl, C₂₋₈alkynyl, halogen, C₁₋₆alkoxy, halo-C₁₋₄alkyl, halo-C₁₋₄alkoxy, cyano, —NH₂, —N(C₁₋₆alkyl)₂, thiol, —SO₂NH₂, —SO₂N(C₁₋₆alkyl)₂, —S(═O)(C₁₋₆alkyl), cycloalkyl, CHR₁ₐ, (C═O)R₁ₐ, OR₁ₐ, N(R_{1b})₂, S(═O)R₁ₐ, S(═O)₂R₁ₐ, O(C═O)R₁ₐ, (C═O)OR₁ₐ, NR_{1b}(C═O)R_{1b}, (C═O)NHR_{1b}, (C═O)N(R_{1b})₂ NR_{1b}(C═O)OR₁ₐ, O(C═O)N(R_{1b})₂, ONR_{1b}(C═NR_{1b})NR_{1b}, NR_{1b}S(═O)₂R₁ₐ or S(═O)₂N(R_{1b})₂, wherein each heterocycle, heteroaryl and aryl are optionally substituted with 1 or 2 substituents each selected from R₅;
each W is independently CH, CR′ or N;
each A is independently absent, CH, CH₂, CRₐ, CHRₐ, N, NH, NRₐ, S, or O, wherein C and N may optionally be substituted with R₄;
each Rₐ is independently H, deuterium, halogen, —CN, —OH, —OR₂, ═O, ═N—OR₂, —SR₂, —S(═O)R₂, —S(═O)₂R², —N(R₂)₂, —NR₂S(═O)(═NR₂)R², —NR₂S(═O)₂R², —S(═O)₂N(R₂)₂, —C(═O)R₂, —OC(═O)R₂, —C(═O)OR₂, —OC(═O)OR₂, —C(═O)N(R₂)₂, —OC(═O)N(R₂)₂, —NR₂C(═O)R₂, —P(═O)(R₂)₂, C₁₋₄alkyl, (C₁₋₄alkyl)₂, halo-C₁₋₆alkyl, C₁₋₆heteroalkyl, C₃₋₈cycloalkyl, C₂₋₇heterocycloalkyl, aryl or monocyclic heteroaryl;
each -- represents either an absent, single, or double bond;
p is 0, 1, 2, 3 or 4;
each Z₁, Z₂, R₂, R′ or R₄ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, halo-C₁₋₄alkoxy, heteroaryl, heterocyclyl, and phenyl;
wherein heteroaryl is a 5-6 membered monocyclic or 9-10 membered bicyclic ring system having 1, 2, 3, or 4 heteroatom ring members selected from N, O, and S,
wherein heterocyclyl is a saturated or partially unsaturated 3-6 membered monocyclic or 9-10 membered bicyclic ring system having 1, 2, or 3 heteroatom ring members selected from N, O, and S, and
wherein each instance of phenyl, heteroaryl or heterocyclyl is optionally substituted with 1, or 2 substituents each selected from R₅;
R₅ is independently selected from halogen, hydroxyl, cyano, nitro, C₁₋₄alkyl, deutero-C₁₋₄alkyl, halo-C₁₋₄alkyl, amino, C₁₋₄alkylamino, (C₁₋₄alkyl)₂amino, aminoC₁₋₄alkyl, hydroxylC₁₋₄alkyl, C₁₋₄alkylcarbonyl, C₁₋₄alkoxy, C₁₋₄alkylthio, halo-C₁₋₄alkoxy, and C₃₋₁₀cycloalkyl
wherein a form of the compound is selected from the group consisting of a pharmaceutically acceptable salt, hydrate, solvate, racemate, enantiomer, diastereomer, stereoisomer, tautomer, and isotope enriched form thereof.

Another aspect of the invention provides a compound having the structure of Formula IXa:

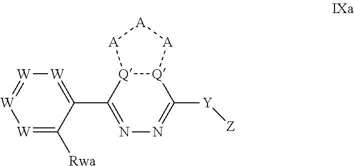

wherein:
each W is independently CH, CR′ or N;
each Q′ is independently N, or C, wherein at least one Q′ is N;
each A is independently CH, CH₂, CRₐ, CR₄, N, NH or NR₄;
Y is NR₁b or a bond;
each R_{wa} is hydrogen, hydroxyl, C₁₋₄alkyl, halo-C₁₋₄alkyl, C₁₋₄alkoxy, or halo-C₁₋₄alkoxy, each R_{1b} is independently hydrogen, C₁₋₄alkyl, deutero-C₁₋₄alkyl, halo-C₁₋₄alkyl or hydroxy-C₁₋₄alkyl;
each Z is heterocyclyl, heteroaryl, aryl, C₃₋₁₀cycloalkyl, C₁₋₄alkyl, deutero-C₁₋₄alkyl, halo-C₁₋₄alkyl, hydroxy-C₁₋₈alkyl, NH(hydroxy-C₁₋₆alkyl), NH(C₁₋₆alkoxy) wherein each Z is optionally substituted with OH, NH₂, —CO₂H, halogen, C₁₋₆alkyl, C₁₋₆haloalkyl, C₁₋₆ hydroxyalkyl, C₂₋₆acyl, C₂₋₆alkanoic acid, C₂₋₆alkanoate ester, or heterocyclyl, and wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S, each optionally substituted with 1, 2, 3, 4, or 5 substituents each selected from R₂;
R₂ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₆alkyl)₂-amino, halo-C₁₋₄alkyl-amino, (halo-C₁₋₆alkyl)₂-amino, hydroxy-C₁₋₄alkyl-amino, C₁₋₄alkoxy-C₁₋₄alkyl-amino, amino-C₁₋₄alkyl, C₁₋₄alkyl-amino-C₁₋₄alkyl, (C₁₋₄alkyl-amino)₂-C₁₋₄alkyl, C₁₋₄alkoxy, halo-C₁₋₄alkoxy, hydroxyl-Chydroxy-C₁₋₄alkoxy, C₁₋₄alkyl-C₁₋₄alkoxy, C₃₋₁₀cycloalkyl, C₃₋₁₀cycloalkyl-amino, C₃₋₁₀cycloalkyl-amino-C₁₋₄alkyl, heteroaryl-C₁₋₄alkyl, heteroaryl-amino, heteroaryl-C₁₋₄alkyl-amino, heterocyclyl, heterocyclyl-C₁₋₄alkyl, heterocyclyl-amino, heterocyclyl-amino-C₁₋₄alkyl, heterocyclyl-C₁₋₄alkoxy, heterocyclyl-amino-C₃₋₁₀cycloalkyl, phenyl, and phenyl-C₁₋₄alkoxy,
wherein heteroaryl is a 5-6 membered monocyclic or 6-10 membered bicyclic ring system having 1, 2, 3, or 4 heteroatom ring members independently selected from N, O, and S,
wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S,
wherein C₃₋₁₀cycloalkyl is a saturated or partially unsaturated 3-7 membered monocyclic ring system,
wherein each instance of phenyl, heteroaryl, heterocyclyl, or C₃₋₁₀cycloalkyl is optionally substituted with 1 or 2 substituents each selected from R₃;
R₃ is independently selected from halogen, hydroxyl, cyano, C₁₋₄alkyl, deutero-C₁₋₄alkyl, halo-C₁₋₄alkyl, amino, C₁₋₄alkoxy, and halo-C₁₋₄alkoxy;
R′ is selected from H, substituted or unsubstituted cycloalkyl, halo-C₁₋₄alkyl, haloalkoxy, halogen, C₁₋₆ alkoxy, cyano, hydroxy-C₁₋₄alkyl or aryl optionally substituted by C₁₋₄alkyl;
each Rₐ is independently H, deuterium, halogen, —CN, —OH, —OR₂, ═O, ═N—OR₂, —SR₂, —S(═O)R₂, —S(═O)₂R², —N(R₂)₂, —NR₂S(═O)(═NR₂)R², —NR₂S(═O)₂R², —S(═O)₂N(R₂)₂, —C(═O)R₂, —OC(═O)R₂, —C(═O)OR₂, —OC(═O)OR₂, —C(═O)N(R₂)₂, —OC(═O)N(R₂)₂, —NR₂C(═O)R₂, —P(═O)(R₂)₂, C₁₋₄alkyl, (C₁₋₄alkyl)₂, halo-C₁₋₆alkyl, C₁₋₆heteroalkyl, C₃₋₈cycloalkyl, C₂₋₇heterocycloalkyl, aryl or monocyclic heteroaryl; and
R₄ is independently selected from halogen, hydroxyl, cyano, or C₁₋₄alkyl;
wherein a form of the compound is selected from the group consisting of a pharmaceutically acceptable salt, hydrate, solvate, racemate, enantiomer, diastereomer, stereoisomer, tautomer, and isotope enriched form thereof.

Another aspect of the invention provides a compound having the structure of Formulae Xa, Xb, Xc, Xd, Xe or Xf:

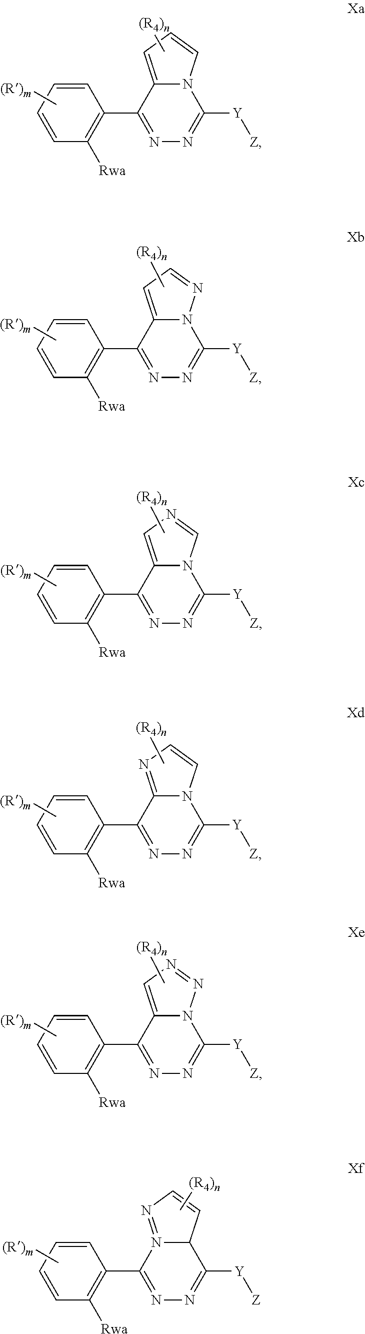

wherein:
each R_{wa} is hydrogen, hydroxyl, C₁₋₄alkyl, halo-C₁₋₄alkyl, C₁₋₄alkoxy, or halo-C₁₋₄alkoxy
R′ is selected from hydrogen, halogen, hydroxyl, cyano, nitro, C₁₋₄alkyl, deutero-C₁₋₄alkyl, halo-C₁₋₄alkyl, amino, C₁₋₄alkyl-amino, (C₁₋₄alkyl)₂-amino, amino-C₁₋₄alkyl, hydroxy-C₁₋₄alkyl, C₁₋₄alkyl-carbonyl, C₁₋₄alkoxy, C₁₋₄alkylthio, halo-C₁₋₄alkoxy, and C₃₋₁₀cycloalkyl; and
R₄ is selected from hydrogen, C₁₋₄ alkyl, halogen, halo-C₁₋₄alkyl;
Y is NR_{1b} or a bond;
each R_{1b} is independently hydrogen, C₁₋₄alkyl, deutero-C₁₋₄alkyl, halo-C₁₋₄alkyl or hydroxy-C₁₋₄alkyl; and
each Z is heterocyclyl, heteroaryl, aryl, C₃₋₁₀cycloalkyl, C₁₋₄alkyl, deutero-C₁₋₄alkyl, halo-C₁₋₄alkyl, hydroxy-C₁₋₄alkyl, NH(hydroxy-C₁₋₆alkyl), NH(C₁₋₆alkoxy) wherein each Z is optionally substituted with OH, NH₂, —CO₂H, halogen, C₁₋₆alkyl, C₁₋₆haloalkyl, C₁₋₆ hydroxyalkyl, C₂₋₆acyl, C₂₋₆alkanoic acid, C₂₋₆alkanoate ester, or heterocyclyl, and wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S, each optionally substituted with 1, 2, 3, 4, or 5 substituents each selected from R₂;
R₂ is independently selected from halogen, hydroxyl, cyano, C₁₋₄alkyl, hydroxy-C₁₋₄alkyl, deuterium-C₁₋₄alkyl, halo-C₁₋₄alkyl, amino, C₁₋₄alkyl-amino, (C₁₋₆alkyl)₂-amino, halo-C₁₋₄alkyl-amino, (halo-C₁₋₆alkyl)₂-amino, hydroxy-C₁₋₄alkyl-amino, C₁₋₄alkoxy-C₁₋₄alkyl-amino, amino-C₁₋₄alkyl, C₁₋₄alkyl-amino-C₁₋₄alkyl, (C₁₋₄alkyl-amino)₂-C₁₋₄alkyl, C₁₋₄alkoxy, halo-C₁₋₄alkoxy, hydroxy-C₁₋₄alkoxy, C₁₋₄alkyl-C₁₋₄alkoxy, C₃₋₁₀cycloalkyl, C₃₋₁₀cycloalkyl-amino, C₃₋₁₀cycloalkyl-amino-C₁₋₄alkyl, heteroaryl-C₁₋₄alkyl, heteroaryl-amino, heteroaryl-C₁₋₄alkyl-amino, heterocyclyl, heterocyclyl-C₁₋₄alkyl, heterocyclyl-amino, heterocyclyl-amino-C₁₋₄alkyl, heterocyclyl-C₁₋₄alkoxy, heterocyclyl-amino-C₃₋₁₀cycloalkyl, phenyl, and phenyl-C₁₋₄alkoxy,
wherein heteroaryl is a 5-6 membered monocyclic or 6-10 membered bicyclic ring system having 1, 2, 3, or 4 heteroatom ring members independently selected from N, O, and S,
wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S,
wherein C₃₋₁₀cycloalkyl is a saturated or partially unsaturated 3-7 membered monocyclic ring system, and
wherein each instance of phenyl, heteroaryl, heterocyclyl, or C₃₋₁₀cycloalkyl is optionally substituted with 1 or 2 substituents each selected from R₃; and
R₃ is independently selected from halogen, hydroxyl, cyano, C₁₋₄alkyl, deuterium-C₁₋₄alkyl, halo-C₁₋₄alkyl, amino, C₁₋₄alkoxy, and halo-C₁₋₄alkoxy;
m is 0, 1, 2, 3, or 4; and
n is 0, 1, 2, or 3;
wherein a form of the compound is selected from the group consisting of a pharmaceutically acceptable salt, hydrate, solvate, racemate, enantiomer, diastereomer, stereoisomer, tautomer, and isotope enriched form thereof.

Another aspect of the invention provides a compound having the structure of Formulae XIa, XIb, XIc:

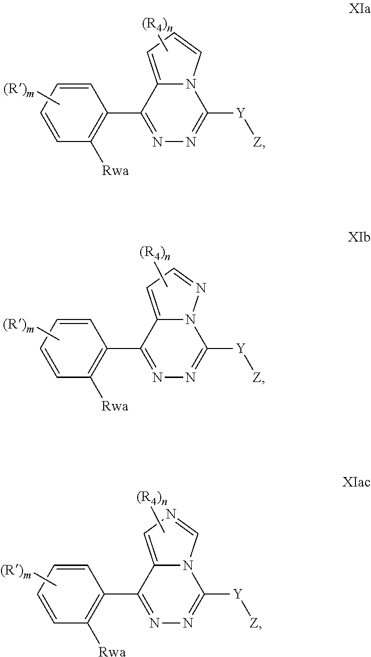

wherein the moiety having the structure

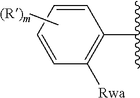

is selected from:

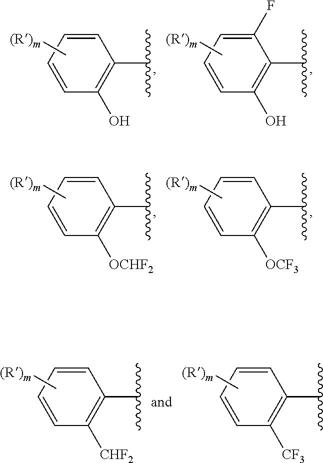

R′ is selected from halogen, hydroxyl, cyano, nitro, C₁₋₄alkyl, deutero-C₁₋₄alkyl, halo-C₁₋₄alkyl, amino, C₁₋₄alkyl-amino, (C₁₋₄alkyl)₂-amino, amino-C₁₋₄alkyl, hydroxy-C₁₋₄alkyl, C₁₋₄alkyl-carbonyl, C₁₋₄alkoxy, C₁₋₄alkylthio, halo-C₁₋₄alkoxy and C₃₋₁₀cycloalkyl;
Y and Z when taken together is selected from:

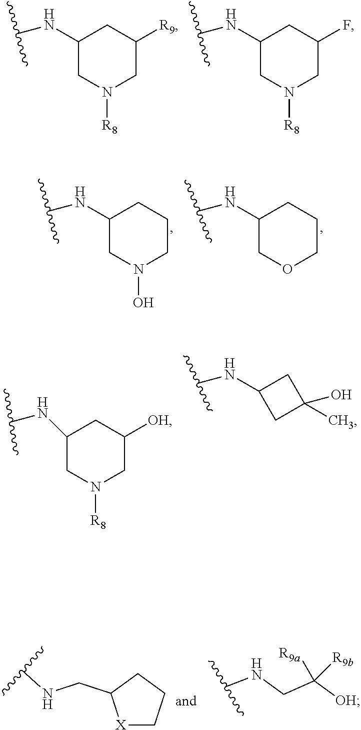

R⁴ is selected from hydrogen, C₁₋₄ alkyl, halogen, halo-C₁₋₄alkyl;
R₈ is selected from C₁₋₄alkyl, CH₂CH₂OH, CH₂CH₂OCF₃, CH₂CH₂OCHF₂ and CH₂CH₂C(CH₃)₂OH;
R₉, R₉ₐ, R_{9b} is selected from hydrogen or C₁₋₄alkyl and C₃₋₆cycloalkyl, wherein C₉a and C₉b can optionally cyclized to form a 3-6 membered ring;
m is 0, 1, 2, 3, or 4;
n is 0, 1, 2, or 3; and
X is selected from 0 or NRs;
wherein a form of the compound is selected from the group consisting of a pharmaceutically acceptable salt, hydrate, solvate, racemate, enantiomer, diastereomer, stereoisomer, tautomer, and isotope enriched form thereof.

Another aspect of the invention provides a compound having the structure of Formulae XIa, XIb, XIc:
wherein the moiety having the structure

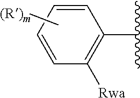

is selected from:

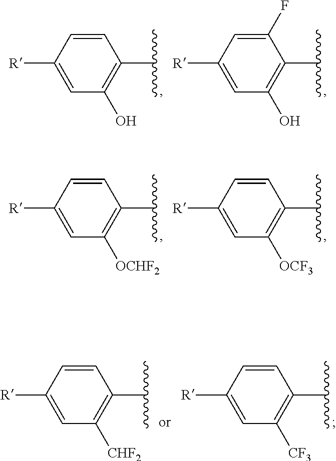

wherein a form of the compound is selected from the group consisting of a pharmaceutically acceptable salt, hydrate, solvate, racemate, enantiomer, diastereomer, stereoisomer, tautomer, and isotope enriched form thereof.

Another aspect includes a compound of Formulae I-XI, wherein Y and Z when taken together is selected from:

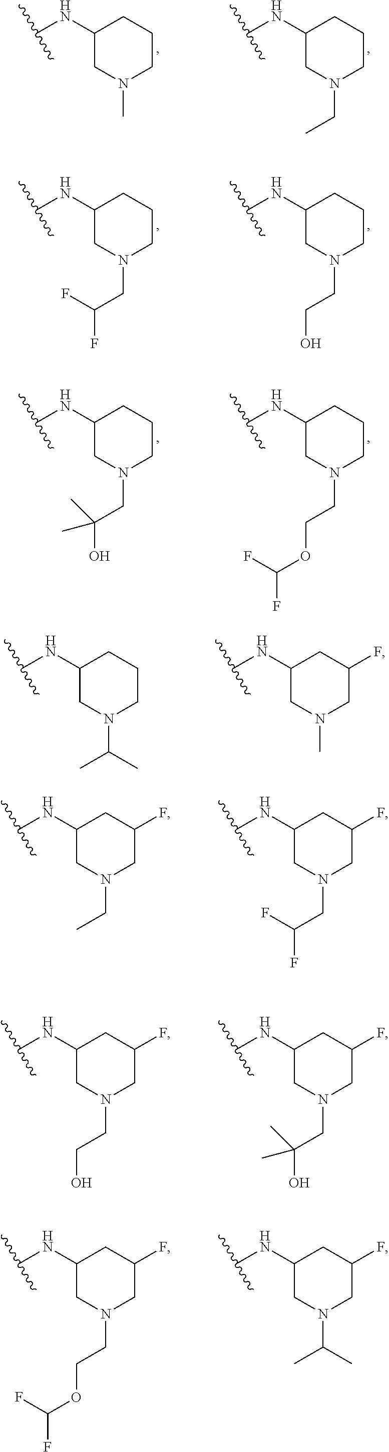

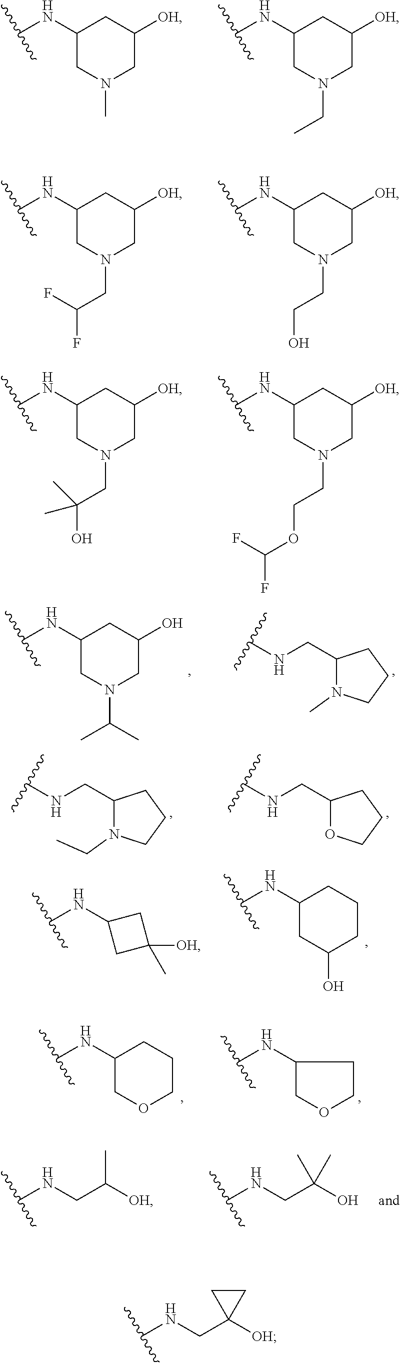

wherein a form of the compound is selected from the group consisting of a pharmaceutically acceptable salt, hydrate, solvate, racemate, enantiomer, diastereomer, stereoisomer, tautomer, and isotope enriched form thereof.

Another aspect of the invention provides any one of the compounds selected from the following:
2-(4-{[(3R)-1-methylpiperidin-3-yl]amino}phthalazin-1-yl)-5-(trifluoromethyl)phenol;
2-(4-{[(3R)-1-methylpiperidin-3-yl]amino}-5,6,7,8-tetrahydrophthalazin-1-yl)-5-(trifluoromethyl)phenol;
2-(4-{[(3R)-1-methylpiperidin-3-yl]amino}-6,7-dihydro-5H-cyclopenta[d]pyridazin-1-yl)-5-(trifluoromethyl)phenol;
2-(4-{[(3R)-1-methylpiperidin-3-yl]amino}thieno[2,3-d]pyridazin-7-yl)-5-(trifluoromethyl)phenol;
2-(8-{[(3R)-1-methylpiperidin-3-yl]amino}pyrido[2,3-d]pyridazin-5-yl)-5-(trifluoromethyl)phenol;
2-{4-[(pyrrolidin-3-yl)amino]phthalazin-1-yl}-5-(trifluoromethyl)phenol;
2-(4-{[(3R)-1-methylpiperidin-3-yl]amino}pyrido[3,4-d]pyridazin-1-yl)-5-(trifluoromethyl)phenol;
2-(8-methyl-4-{[(3R)-1-methylpiperidin-3-yl]amino}phthalazin-1-yl)-5-(trifluoromethyl)phenol;
2-(7-fluoro-4-{[(3R)-1-methylpiperidin-3-yl]amino}phthalazin-1-yl)-5-(trifluoromethyl)phenol;
2-(4-{[(3R)-1-methylpiperidin-3-yl]amino}phthalazin-1-yl)-5-(2H-1,2,3-triazol-2-yl)phenol;
2-(4-{[(3R)-piperidin-3-yl]amino}-5,6,7,8-tetrahydrophthalazin-1-yl)-5-(trifluoromethyl)phenol;
2-(4-{[(3 S)-1-methylpiperidin-3-yl]amino}phthalazin-1-yl)-5-(trifluoromethyl)phenol;
5-chloro-2-(4-{[(3R)-1-methylpiperidin-3-yl]amino}phthalazin-1-yl)phenol;
3-fluoro-2-(4-{[(3R)-1-methylpiperidin-3-yl]amino}phthalazin-1-yl)phenol;
4-(4-{[(3R)-1-methylpiperidin-3-yl]amino}phthalazin-1-yl)benzene-1,3-diol;
5-fluoro-2-(4-{[(3R)-1-methylpiperidin-3-yl]amino}phthalazin-1-yl)phenol;
4-methyl-2-(4-{[(3R)-1-methylpiperidin-3-yl]amino}phthalazin-1-yl)phenol;
4-fluoro-5-methyl-2-(4-{[(3R)-1-methylpiperidin-3-yl]amino}phthalazin-1-yl)phenol;
2-(4-{[(3R)-1-ethylpiperidin-3-yl]amino}-5,6,7,8-tetrahydrophthalazin-1-yl)-5-(trifluoromethyl)phenol;
2-(4-{[(3R)-piperidin-3-yl]amino}phthalazin-1-yl)-5-(trifluoromethyl)phenol;
2-(4-{[(3R)-piperidin-3-yl]oxy}phthalazin-1-yl)-5-(trifluoromethyl)phenol;
2-(4-{[(3 S)-1-methylpiperidin-3-yl]oxy}phthalazin-1-yl)-5-(trifluoromethyl)phenol;
2-(8-methyl-4-{[(3R)-piperidin-3-yl]amino}-5,6,7,8-tetrahydrophthalazin-1-yl)-5-(trifluoromethyl)phenol;
3-hydroxy-4-(4-{[(3R)-1-methylpiperidin-3-yl]amino}phthalazin-1-yl)benzonitrile;
5-methyl-2-(4-{[(3R)-1-methylpiperidin-3-yl]amino}phthalazin-1-yl)phenol;
2-(4-{[(3R)-1-methylpyrrolidin-3-yl]amino}-5,6,7,8-tetrahydrophthalazin-1-yl)-5-(trifluoromethyl)phenol;
2-methyl-6-(4-{[(3R)-1-methylpiperidin-3-yl]amino}phthalazin-1-yl)phenol;
2-fluoro-6-(4-{[(3R)-1-methylpiperidin-3-yl]amino}phthalazin-1-yl)phenol;
2-(4-{[(3R)-1-methylpiperidin-3-yl]oxy}phthalazin-1-yl)-5-(trifluoromethyl)phenol;
2-{4-[(piperidin-3-yl)methyl]phthalazin-1-yl}-5-(trifluoromethyl)phenol;
2-(4-{[(3R)-piperidin-3-yl]amino}-6,7,8,9-tetrahydro-5H-cyclohepta[d]pyridazin-1-yl)-5-(trifluoromethyl)phenol;
5-(5-methyl-1,2,4-thiadiazol-3-yl)-2-(4-{[(3R)-piperidin-3-yl]amino}phthalazin-1-yl)phenol;
2-(4-{[(3R)-1-methylpiperidin-3-yl]amino}phthalazin-1-yl)phenol;
2-(4-{[(3R)-1-(propan-2-yl)piperidin-3-yl]amino}-5,6,7,8-tetrahydrophthalazin-1-yl)-5-(trifluoromethyl)phenol;
2-(4-{[(3R)-piperidin-3-yl]amino}pyrido[3,4-d]pyridazin-1-yl)-5-(trifluoromethyl)phenol;
2-(4-{[(3R)-1-methylpiperidin-3-yl]amino}-6,7,8,9-tetrahydro-5H-cyclohepta[d]pyridazin-1-yl)-5-(trifluoromethyl)phenol;
2-(4-{[(3R)-1-methylpiperidin-3-yl]amino}phthalazin-1-yl)-5-(1H-1,2,3-triazol-1-yl)phenol;
2-(4-{[(3R)-1-methylpiperidin-3-yl]amino}phthalazin-1-yl)-5-(5-methyl-1,2,4-thiadiazol-3-yl)phenol;
4-(4-{[(3R)-1-methylpiperidin-3-yl]amino}phthalazin-1-yl)[1,1′-biphenyl]-3-ol;
3-(4-{[(3R)-1-methylpiperidin-3-yl]amino}phthalazin-1-yl)[1,1′-biphenyl]-4-ol;
4,5-dimethyl-2-(4-{[(3R)-1-methylpiperidin-3-yl]amino}phthalazin-1-yl)phenol;
2,3-dimethyl-6-(4-{[(3R)-1-methylpiperidin-3-yl]amino}phthalazin-1-yl)phenol;
2-(4-{[(3R)-1-ethylpiperidin-3-yl]amino}pyrido[3,4-d]pyridazin-1-yl)-5-(trifluoromethyl)phenol;
2-{4-[(1-methylpiperidin-3-yl)methyl]phthalazin-1-yl}-5-(trifluoromethyl)phenol;
2-(7,7-dimethyl-4-{[(3R)-piperidin-3-yl]amino}-6,7-dihydro-5H-cyclopenta[d]pyridazin-1-yl)-5-(trifluoromethyl)phenol;
2-(7,7-dimethyl-4-{[(3R)-1-methylpiperidin-3-yl]amino}-6,7-dihydro-5H-cyclopenta[d]pyridazin-1-yl)-5-(trifluoromethyl)phenol;
2-(4-{methyl[(3R)-1-methylpiperidin-3-yl]amino}-5,6,7,8-tetrahydrophthalazin-1-yl)-5-(trifluoromethyl)phenol;
2-(4-{[(3R)-1-(2-hydroxyethyl)piperidin-3-yl]amino}phthalazin-1-yl)-5-(trifluoromethyl)phenol;
5-cyclopropyl-2-(4-{[(3R)-1-methylpiperidin-3-yl]amino}phthalazin-1-yl)phenol;
3-methyl-2-(4-{[(3R)-1-methylpiperidin-3-yl]amino}phthalazin-1-yl)phenol;
2-fluoro-3-methyl-6-(4-{[(3R)-1-methylpiperidin-3-yl]amino}phthalazin-1-yl)phenol;
5-(5,6-dihydro-2H-pyran-3-yl)-2-(4-{[(3R)-1-methylpiperidin-3-yl]amino}phthalazin-1-yl)phenol;
(3S,5R)-5-({4-[2-hydroxy-4-(trifluoromethyl)phenyl]phthalazin-1-yl}amino)piperidin-3-ol;
2-(1-{[(3R)-1-methylpiperidin-3-yl]amino}pyrido[3,4-d]pyridazin-4-yl)-5-(trifluoromethyl)phenol;
2-(4-{[(3R)-1-(2-hydroxyethyl)piperidin-3-yl]amino}pyrido[3,4-d]pyridazin-1-yl)-5-(trifluoromethyl)phenol;
5-(3,6-dihydro-2H-pyran-4-yl)-2-(4-{[(3R)-1-methylpiperidin-3-yl]amino}phthalazin-1-yl)phenol;
5-(2,5-dihydrofuran-3-yl)-2-(4-{[(3R)-1-methylpiperidin-3-yl]amino}phthalazin-1-yl)phenol;
5-(1-methyl-2,5-dihydro-1H-pyrrol-3-yl)-2-(4-{[(3R)-1-methylpiperidin-3-yl]amino}phthalazin-1-yl)phenol;
5-chloro-2-(1-{[(3R)-1-methylpiperidin-3-yl]amino}pyrido[3,4-d]pyridazin-4-yl)phenol;
5-chloro-2-(4-{[(3R)-1-methylpiperidin-3-yl]amino}pyrido[3,4-d]pyridazin-1-yl)phenol;
2-(4-{[(3R)-1-methylpiperidin-3-yl]amino}phthalazin-1-yl)-5-(oxolan-3-yl)phenol;
2-(4-{[(3R)-1-(2-hydroxyethyl)piperidin-3-yl]amino}-5,6,7,8-tetrahydrophthalazin-1-yl)-5-(trifluoromethyl)phenol;
(3S,5R)-5-({4-[2-hydroxy-4-(trifluoromethyl)phenyl]phthalazin-1-yl}amino)-1-methylpiperidin-3-ol;
1-[3-({4-[2-hydroxy-4-(trifluoromethyl)phenyl]phthalazin-1-yl}amino)piperidin-1-yl]ethan-1-one;
ethyl [(3R)-3-({4-[2-hydroxy-4-(trifluoromethyl)phenyl]phthalazin-1-yl}amino)piperidin-1-yl]acetate;
2-(5-{[(3R)-1-methylpiperidin-3-yl]amino}pyrido[2,3-d]pyridazin-8-yl)-5-(trifluoromethyl)phenol;
5-chloro-2-(8-{[(3R)-1-methylpiperidin-3-yl]amino}pyrido[2,3-d]pyridazin-5-yl)phenol;
2-[4-({[(2S)-pyrrolidin-2-yl]methyl}amino)phthalazin-1-yl]-5-(trifluoromethyl)phenol;
5-chloro-3-fluoro-2-(4-{[(3R)-1-methylpiperidin-3-yl]amino}phthalazin-1-yl)phenol;
5-methyl-2-(4-{[(3R)-1-methylpiperidin-3-yl]amino}pyrido[3,4-d]pyridazin-1-yl)phenol;
3-fluoro-2-(4-{[(3R)-1-methylpiperidin-3-yl]amino}pyrido[3,4-d]pyridazin-1-yl)phenol;
2-methoxy-6-(4-{[(3R)-1-methylpiperidin-3-yl]amino}pyrido[3,4-d]pyridazin-1-yl)phenol;
4-hydroxy-5-(4-{[(3R)-1-methylpiperidin-3-yl]amino}phthalazin-1-yl)pyridine-2-carbonitrile;
3-(4-{[(3R)-1-methylpiperidin-3-yl]amino}phthalazin-1-yl)-6-(trifluoromethyl)pyridin-2-01;
2-chloro-6-(4-{[(3R)-1-methylpiperidin-3-yl]amino}pyrido[3,4-d]pyridazin-1-yl)phenol;
2-cyclopropyl-6-(4-{[(3R)-1-methylpiperidin-3-yl]amino}pyrido[3,4-d]pyridazin-1-yl)phenol;
6-methyl-3-(4-{[(3R)-1-methylpiperidin-3-yl]amino}pyrido[3,4-d]pyridazin-1-yl)pyridin-2-ol;
5-(4-{[(3R)-1-methylpiperidin-3-yl]amino}phthalazin-1-yl)-2-(trifluoromethyl)pyridin-4-01;
6-hydroxy-5-(4-{[(3R)-1-methylpiperidin-3-yl]amino}phthalazin-1-yl)pyridine-2-carbonitrile;
5-chloro-2-(4-{[(3R)-1-methylpiperidin-3-yl]amino}phthalazin-1-yl)pyridin-3-ol;
2-methyl-6-(4-{[(3R)-1-methylpiperidin-3-yl]amino}pyrido[3,4-d]pyridazin-1-yl)phenol;
(3R,5R)-5-({4-[2-hydroxy-4-(trifluoromethyl)phenyl]phthalazin-1-yl}amino)piperidin-3-ol;
(3R,5R)-5-({4-[2-hydroxy-4-(trifluoromethyl)phenyl]phthalazin-1-yl}amino)-1-methylpiperidin-3-ol;
2-(4-{[(3R)-1-methylpiperidin-3-yl]amino}furo[2,3-d]pyridazin-7-yl)-5-(trifluoromethyl)phenol;
5-methyl-2-(8-{[(3R)-1-methylpiperidin-3-yl]amino}pyrido[2,3-d]pyridazin-5-yl)phenol;
2-[4-({[(2S)-1-methylpyrrolidin-2-yl]methyl}amino)phthalazin-1-yl]-5-(trifluoromethyl)phenol;
4-fluoro-5-methyl-2-(4-{[(3R)-1-methylpiperidin-3-yl]amino}pyrido[3,4-d]pyridazin-1-yl)phenol;
5-cyclopropyl-2-(4-{[(3R)-1-methylpiperidin-3-yl]amino}pyrido[3,4-d]pyridazin-1-yl)phenol;
5-methyl-2-(4-{[(3R)-1-methylpiperidin-3-yl]amino}phthalazin-1-yl)pyridin-3-ol;
5-cyclopropyl-2-(4-{[(3R)-1-methylpiperidin-3-yl]amino}phthalazin-1-yl)pyridin-3-ol;
3-hydroxy-4-(4-{[(3R)-1-methylpiperidin-3-yl]amino}pyrido[3,4-d]pyridazin-1-yl)benzonitrile;
2-(4-{[(3R)-1-methylpiperidin-3-yl]amino}phthalazin-1-yl)-5-(pyrrolidin-1-yl)phenol;
2-(4-{[(3R)-1-methylpiperidin-3-yl]amino}phthalazin-1-yl)-5-(1-methyl-1H-pyrrol-3-yl)phenol;
2-{4-[(1-methylazepan-3-yl)amino]phthalazin-1-yl}-5-(trifluoromethyl)phenol;
2-(4-{[(3 S)-1-methylpiperidin-3-yl]methyl}phthalazin-1-yl)-5-(trifluoromethyl)phenol;
2-(4-{[(3R)-1-methylpiperidin-3-yl]methyl}phthalazin-1-yl)-5-(trifluoromethyl)phenol;
[(3R)-3-({4-[2-hydroxy-4-(trifluoromethyl)phenyl]phthalazin-1-yl}amino)piperidin-1-yl]acetic acid;
5-methyl-2-(1-{[(3R)-1-methylpiperidin-3-yl]amino}pyrido[3,4-d]pyridazin-4-yl)phenol;
2-{1-[(piperidin-3-yl)methyl]pyrido[3,4-d]pyridazin-4-yl}-5-(trifluoromethyl)phenol;
2-(1-{[(3R)-1-(2-hydroxyethyl)piperidin-3-yl]amino}pyrido[3,4-d]pyridazin-4-yl)-5-(trifluoromethyl)phenol;
2-(4-{[(3R)-1-methylpiperidin-3-yl]amino}pyrido[3,4-d]pyridazin-1-yl)-5-(trifluoromethoxy)phenol;
3-methyl-2-(4-{[(3R)-1-methylpiperidin-3-yl]amino}pyrido[3,4-d]pyridazin-1-yl)-5-(trifluoromethyl)phenol;
2-(8-chloro-4-{[(3R)-1-methylpiperidin-3-yl]amino}pyrrolo[1,2-d][1,2,4]triazin-1-yl)-5-(trifluoromethyl)phenol;
3-methyl-2-(4-{[(3R)-1-methylpiperidin-3-yl]amino}pyrido[3,4-d]pyridazin-1-yl)phenol;
2-(4-{[(3R)-1-methylpiperidin-3-yl]amino}pyrrolo[1,2-d][1,2,4]triazin-1-yl)-5-(trifluoromethyl)phenol;
2-(4-{[(3R)-1-methylpiperidin-3-yl]amino}phthalazin-1-yl)-5-(1-methylpyrrolidin-3-yl)phenol;
2-(4-{[2-(morpholin-4-yl)ethyl]amino}phthalazin-1-yl)-5-(trifluoromethyl)phenol;
2-{4-[(azetidin-3-yl)amino]phthalazin-1-yl}-5-(trifluoromethyl)phenol;
2-[1-({[(2S)-pyrrolidin-2-yl]methyl}amino)pyrido[3,4-d]pyridazin-4-yl]-5-(trifluoromethyl)phenol;
2-[4-({[(2S)-pyrrolidin-2-yl]methyl}amino)pyrido[3,4-d]pyridazin-1-yl]-5-(trifluoromethyl)phenol;
2-[4-({[(2S)-1-methylpyrrolidin-2-yl]methyl}amino)pyrido[3,4-d]pyridazin-1-yl]-5-(trifluoromethyl)phenol;
2-[4-({[(2S)-1-ethylpyrrolidin-2-yl]methyl}amino)pyrido[3,4-d]pyridazin-1-yl]-5-(trifluoromethyl)phenol;
2-(4-{[2-(piperidin-1-yl)ethyl]amino}phthalazin-1-yl)-5-(trifluoromethyl)phenol;
2-[1-(4-methylpiperazin-1-yl)pyrido[3,4-d]pyridazin-4-yl]-5-(trifluoromethyl)phenol;
2-(4-methyl-5-{[(3R)-1-methylpiperidin-3-yl]amino}-3,4-dihydro-2H-pyridazino[4,5-b][1,4]oxazin-8-yl)-5-(trifluoromethyl)phenol;
2-{1-[(1-methylpiperidin-3-yl)methyl]pyrido[3,4-d]pyridazin-4-yl}-5-(trifluoromethyl)phenol;
1-[2-methoxy-4-(trifluoromethyl)phenyl]-N-[(3R)-1-methylpiperidin-3-yl]pyrido[3,4-d]pyridazin-4-amine;
4-[2-(difluoromethoxy)-4-(trifluoromethyl)phenyl]-N-[(3R)-1-methylpiperidin-3-yl]phthalazin-1-amine;
1-[2-(difluoromethoxy)-4-(trifluoromethyl)phenyl]-N-[(3R)-1-methylpiperidin-3-yl]pyrido[3,4-d]pyridazin-4-amine;
4-[2-(difluoromethoxy)-4-(trifluoromethyl)phenyl]-N-[(3R)-1-methylpiperidin-3-yl]pyrido[3,4-d]pyridazin-1-amine;
4-[2-amino-4-(trifluoromethyl)phenyl]-N-[(3R)-1-methylpiperidin-3-yl]phthalazin-1-amine;
N-[2-(4-{[(3R)-1-methylpiperidin-3-yl]amino}phthalazin-1-yl)-5-(trifluoromethyl)phenyl]methanesulfonamide;
4-[2-(methylamino)-4-(trifluoromethyl)phenyl]-N-[(3R)-1-methylpiperidin-3-yl]phthalazin-1-amine;
N-[2-(4-{[(3R)-1-methylpiperidin-3-yl]amino}phthalazin-1-yl)-5-(trifluoromethyl)phenyl]acetamide;
4-[2-fluoro-4-(trifluoromethyl)phenyl]-N-[(3R)-1-methylpiperidin-3-yl]phthalazin-1-amine;
1-[2-fluoro-4-(trifluoromethyl)phenyl]-N-[(3R)-1-methylpiperidin-3-yl]pyrido[3,4-d]pyridazin-4-amine;
1-(4-chloro-2-fluorophenyl)-N-[(3R)-1-methylpiperidin-3-yl]pyrido[3,4-d]pyridazin-4-amine;
2-(4-{[(3R)-1-methylpiperidin-3-yl]amino}pyrido[3,4-d]pyridazin-1-yl)-5-(trifluoromethyl)benzonitrile;
2-(4-{[(3R)-1-methylpiperidin-3-yl]amino}phthalazin-1-yl)-5-(trifluoromethyl)benzonitrile;
1-[4-chloro-2-(difluoromethoxy)phenyl]-N-[(3R)-1-methylpiperidin-3-yl]pyrido[3,4-d]pyridazin-4-amine;
4-[4-chloro-2-(difluoromethoxy)phenyl]-N-[(3R)-1-methylpiperidin-3-yl]phthalazin-1-amine;
1-[2-(difluoromethoxy)-4-methylphenyl]-N-[(3R)-1-methylpiperidin-3-yl]pyrido[3,4-d]pyridazin-4-amine;
4-[2-(difluoromethoxy)-4-methylphenyl]-N-[(3R)-1-methylpiperidin-3-yl]phthalazin-1-amine;
5-methyl-2-(4-{[(3R)-1-methylpiperidin-3-yl]amino}pyrido[3,4-d]pyridazin-1-yl)benzonitrile;
2-(4-{[(3R)-1-methylpiperidin-3-yl]amino}pyrido[3,4-d]pyridazin-1-yl)-5-(2H-1,2,3-triazol-2-yl)phenol;
1-[2-(difluoromethyl)-4-(trifluoromethyl)phenyl]-N-[(3R)-1-methylpiperidin-3-yl]pyrido[3,4-d]pyridazin-4-amine;
3-chloro-2-(4-{[(3S)-1-methylpiperidin-3-yl]amino}pyrido[3,4-d]pyridazin-1-yl)phenol;
3-hydroxy-2-(4-{[(3R)-1-methylpiperidin-3-yl]amino}pyrido[3,4-d]pyridazin-1-yl)benzonitrile;
3,5-dimethyl-2-(4-{[(3R)-1-methylpiperidin-3-yl]amino}pyrido[3,4-d]pyridazin-1-yl)phenol;
5-chloro-3-methyl-2-(4-{[(3R)-1-methylpiperidin-3-yl]amino}pyrido[3,4-d]pyridazin-1-yl)phenol;
1-[2-chloro-4-(trifluoromethyl)phenyl]-N-[(3R)-1-methylpiperidin-3-yl]pyrido[3,4-d]pyridazin-4-amine;
4-[2-methoxy-4-(trifluoromethyl)phenyl]-1-methyl-N-[(3R)-1-methylpiperidin-3-yl]-1H-pyrazolo[3,4-d]pyridazin-7-amine;
1-[2-(2,5-dihydrofuran-3-yl)-4-(trifluoromethyl)phenyl]-N-[(3R)-1-methylpiperidin-3-yl]pyrido[3,4-d]pyridazin-4-amine;
2-(1-methyl-7-{[(3R)-1-methylpiperidin-3-yl]amino}-1H-pyrazolo[3,4-d]pyridazin-4-yl)-5-(trifluoromethyl)phenol;
2-[4-({2-[(3R)-3-methylmorpholin-4-yl]ethyl}amino)phthalazin-1-yl]-5-(trifluoromethyl)phenol;
2-[4-({2-[(3 S)-3-methylmorpholin-4-yl]ethyl}amino)phthalazin-1-yl]-5-(trifluoromethyl)phenol;
2-(4-{[2-(piperazin-1-yl)ethyl]amino}phthalazin-1-yl)-5-(trifluoromethyl)phenol;
2-(4-{[(azetidin-2-yl)methyl]amino}phthalazin-1-yl)-5-(trifluoromethyl)phenol;
2-(4-{[(3R,5R)-5-fluoro-1-methylpiperidin-3-yl]amino}phthalazin-1-yl)-5-(trifluoromethyl)phenol;
2-{1-[3-(dimethylamino)piperidin-1-yl]pyrido[3,4-d]pyridazin-4-yl}-5-(trifluoromethyl)phenol;
4-[2-methoxy-4-(trifluoromethyl)phenyl]-1-(piperazin-1-yl)pyrido[3,4-d]pyridazine;
2-(1-methyl-8-{[(3R)-1-methylpiperidin-3-yl]amino}-1,2,3,4-tetrahydropyrido[2,3-d]pyridazin-5-yl)-5-(trifluoromethyl)phenol;
5-chloro-2-(1-methyl-8-{[(3S)-1-methylpiperidin-3-yl]amino}-1,2,3,4-tetrahydropyrido[2,3-d]pyridazin-5-yl)phenol;
2-(4-{[(3R)-1-methylpiperidin-3-yl]amino}pyrido[3,4-d]pyridazin-1-yl)-5-(trifluoromethyl)benzene-1-sulfonamide;
5-chloro-2-(4-{[(3R)-1-methylpiperidin-3-yl]amino}pyrido[3,4-d]pyridazin-1-yl)benzonitrile;
1-[4-cyclopropyl-2-(trifluoromethyl)phenyl]-N-[(3R)-1-methylpiperidin-3-yl]pyrido[3,4-d]pyridazin-4-amine;
3-methyl-5-(4-{[(3R)-1-methylpiperidin-3-yl]amino}phthalazin-1-yl)pyridin-4-ol;
4-(2-methoxy-6-methylpyridin-3-yl)-N-[(3R)-1-methylpiperidin-3-yl]phthalazin-1-amine;
2-(4-{[2-(4-methylpiperazin-1-yl)ethyl]amino}phthalazin-1-yl)-5-(trifluoromethyl)phenol;
1-{4-[2-methoxy-4-(trifluoromethyl)phenyl]pyrido[3,4-d]pyridazin-1-yl}-N,N-dimethylpiperidin-3-amine;
2-amino-3-chloro-6-(4-{[(3R)-1-methylpiperidin-3-yl]amino}phthalazin-1-yl)phenol;
2-(4-{[(3 S,5S)-5-fluoro-1-methylpiperidin-3-yl]amino}phthalazin-1-yl)-5-(trifluoromethyl)phenol;
4-(2,3-difluoro-4-methylphenyl)-N-[(3R)-1-methylpiperidin-3-yl]phthalazin-1-amine;
N-[(3R)-1-methylpiperidin-3-yl]-1-[4-(trifluoromethyl)phenyl]pyrido[3,4-d]pyridazin-4-amine;
3-(difluoromethoxy)-4-(4-{[(3R)-1-methylpiperidin-3-yl]amino}pyrido[3,4-d]pyridazin-1-yl)benzonitrile;
1-[2-(difluoromethoxy)-3-methylphenyl]-N-[(3R)-1-methylpiperidin-3-yl]pyrido[3,4-d]pyridazin-4-amine;
N-methyl-2-(4-{[(3R)-1-methylpiperidin-3-yl]amino}pyrido[3,4-d]pyridazin-1-yl)-5-(trifluoromethyl)benzene-1-sulfonamide;
5-chloro-3-fluoro-2-(4-{[(3R)-1-methylpiperidin-3-yl]amino}pyrido[3,4-d]pyridazin-1-yl)phenol;
1-[2-(difluoromethoxy)-4-methoxyphenyl]-N-[(3R)-1-methylpiperidin-3-yl]pyrido[3,4-d]pyridazin-4-amine;
5-chloro-2-(4-{[(3R)-1-methylpiperidin-3-yl]amino}pyrido[3,4-d]pyridazin-1-yl)pyridin-3-ol;
1-[2-(methoxymethoxy)-4-(1,3-oxazol-2-yl)phenyl]-N-[(3S)-1-methylpiperidin-3-yl]pyrido[3,4-d]pyridazin-4-amine;
2-(4-{[(1-methyl-1H-pyrazol-4-yl)methyl]amino}pyrido[3,4-d]pyridazin-1-yl)-5-(trifluoromethyl)phenol;
5-methoxy-2-(4-{[(3R)-1-methylpiperidin-3-yl]amino}pyrido[3,4-d]pyridazin-1-yl)phenol;
2-(4-{[(3 S)-1-methylpiperidin-3-yl]amino}pyrido[3,4-d]pyridazin-1-yl)-5-(1,3-oxazol-2-yl)phenol;
1-[2,4-bis(trifluoromethyl)phenyl]-N-[(3R)-1-methylpiperidin-3-yl]pyrido[3,4-d]pyridazin-4-amine;
1-[2-(difluoromethoxy)-5-fluoro-4-methylphenyl]-N-[(3R)-1-methylpiperidin-3-yl]pyrido[3,4-d]pyridazin-4-amine;
3-fluoro-5-methyl-2-(4-{[(3R)-1-methylpiperidin-3-yl]amino}pyrido[3,4-d]pyridazin-1-yl)phenol;
2-{4-[(1-methyl-1H-pyrazol-4-yl)amino]pyrido[3,4-d]pyridazin-1-yl}-5-(trifluoromethyl)phenol;
1-[4-methoxy-2-(trifluoromethyl)phenyl]-N-[(3R)-1-methylpiperidin-3-yl]pyrido[3,4-d]pyridazin-4-amine;
2-(4-{[(3R)-1-methylpiperidin-3-yl]amino}pyrido[3,4-d]pyridazin-1-yl)-5-(trifluoromethyl)benzamide;
5-[2-(difluoromethoxy)-4-(trifluoromethyl)phenyl]-N-[(3R)-1-methylpiperidin-3-yl]pyrido[2,3-d]pyridazin-8-amine;
8-[2-(difluoromethoxy)-4-(trifluoromethyl)phenyl]-N-[(3R)-1-methylpiperidin-3-yl]pyrido[2,3-d]pyridazin-5-amine;
2-{1-[(1-methylazepan-3-yl)amino]pyrido[3,4-d]pyridazin-4-yl}-5-(trifluoromethyl)phenol;
2-(4-{[(3 S)-1-methylazepan-3-yl]amino}pyrido[3,4-d]pyridazin-1-yl)-5-(trifluoromethyl)phenol;
2-(4-{[(3R)-1-methylazepan-3-yl]amino}pyrido[3,4-d]pyridazin-1-yl)-5-(trifluoromethyl)phenol;
N-[(3R)-1-methylpiperidin-3-yl]-1-{2-[(oxetan-3-yl)oxy]-4-(trifluoromethyl)phenyl}pyrido[3,4-d]pyridazin-4-amine;
N-[(3R)-1-methylpiperidin-3-yl]-1-[2-(1-methyl-1H-pyrazol-4-yl)-4-(trifluoromethyl)phenyl]pyrido[3,4-d]pyridazin-4-amine;
[2-(4-{[(3R)-1-methylpiperidin-3-yl]amino}pyrido[3,4-d]pyridazin-1-yl)-5-(trifluoromethyl)phenyl]methanol;
1-[2-(difluoromethoxy)-4-(trifluoromethoxy)phenyl]-N-[(3R)-1-methylpiperidin-3-yl]pyrido[3,4-d]pyridazin-4-amine;
1-[2-(methanesulfonyl)-4-(trifluoromethyl)phenyl]-N-[(3R)-1-methylpiperidin-3-yl]pyrido[3,4-d]pyridazin-4-amine;
N-[(3R)-1-methylpiperidin-3-yl]-1-[2-(1H-pyrazol-4-yl)-4-(trifluoromethyl)phenyl]pyrido[3,4-d]pyridazin-4-amine;
1-[2-cyclopropyl-4-(trifluoromethyl)phenyl]-N-[(3R)-1-methylpiperidin-3-yl]pyrido[3,4-d]pyridazin-4-amine;
5-methyl-2-(4-{[(3R)-1-methylpiperidin-3-yl]amino}pyrido[3,4-d]pyridazin-1-yl)pyridin-3-ol;
5-cyclopropyl-3-fluoro-2-(4-{[(3R)-1-methylpiperidin-3-yl]amino}pyrido[3,4-d]pyridazin-1-yl)phenol;
3-fluoro-2-(4-{[(3R)-1-methylpiperidin-3-yl]amino}pyrido[3,4-d]pyridazin-1-yl)-5-(trifluoromethyl)phenol;
1-(4-chloro-2-fluoro-6-methylphenyl)-N-[(3R)-1-methylpiperidin-3-yl]pyrido[3,4-d]pyridazin-4-amine;
N-[(3R)-1-azabicyclo[2.2.2]octan-3-yl]-1-[2-methoxy-4-(trifluoromethyl)phenyl]pyrido[3,4-d]pyridazin-4-amine;
2-(4-{[(3R)-1-methylpiperidin-3-yl]amino}pyrido[3,4-d]pyridazin-1-yl)-5-(pyrimidin-2-yl)phenol;
1-{4-[2-methoxy-4-(trifluoromethyl)phenyl]phthalazin-1-yl}-N,N-dimethylpiperidin-4-amine;
N-[(3R)-1-methylpiperidin-3-yl]-1-[5-(trifluoromethyl)[1,1′-biphenyl]-2-yl]pyrido[3,4-d]pyridazin-4-amine;
2-(4-{3-[(dimethylamino)methyl]pyrrolidin-1-yl}phthalazin-1-yl)-5-(trifluoromethyl)phenol;
1-[2-(difluoromethoxy)-6-(trifluoromethyl)pyridin-3-yl]-N-[(3R)-1-methylpiperidin-3-yl]pyrido[3,4-d]pyridazin-4-amine;
N-[(3R)-1-methylpiperidin-3-yl]-1-(2,4,6-trimethylphenyl)pyrido[3,4-d]pyridazin-4-amine;
2-[(3R)-3-({1-[2-(difluoromethoxy)-4-(trifluoromethyl)phenyl]pyrido[3,4-d]pyridazin-4-yl}amino)piperidin-1-yl]ethan-1-ol;
1-[2,4-di(propan-2-yl)phenyl]-N-[(3R)-1-methylpiperidin-3-yl]pyrido[3,4-d]pyridazin-4-amine;
1-(3-cyclopropyl-6-fluoro-2-methoxyphenyl)-N-[(3R)-1-methylpiperidin-3-yl]pyrido[3,4-d]pyridazin-4-amine;
6-chloro-3-fluoro-2-(4-{[(3R)-1-methylpiperidin-3-yl]amino}pyrido[3,4-d]pyridazin-1-yl)phenol;
6-cyclopropyl-3-fluoro-2-(4-{[(3R)-1-methylpiperidin-3-yl]amino}pyrido[3,4-d]pyridazin-1-yl)phenol;
6-chloro-3-fluoro-2-(5-{[(3R)-1-methylpiperidin-3-yl]amino}pyrido[2,3-d]pyridazin-8-yl)phenol;
2-{4-[(4-methyl-4-azaspiro[2.5]octan-7-yl)amino]pyrido[3,4-d]pyridazin-1-yl}-5-(trifluoromethyl)phenol;
1-[2-methoxy-4-(trifluoromethyl)phenyl]-N-(8-methyl-8-azabicyclo[3.2.1]octan-3-yl)pyrido[3,4-d]pyridazin-4-amine;
N-[(3R)-1-methylpiperidin-3-yl]-1-[2-(2,2,2-trifluoroethyl)-4-(trifluoromethyl)phenyl]pyrido[3,4-d]pyridazin-4-amine;
2-(4-{[(3R)-1-cyclobutylpiperidin-3-yl]amino}pyrido[3,4-d]pyridazin-1-yl)-5-(trifluoromethyl)phenol;
1-(2,4-dimethylphenyl)-N-[(3R)-1-methylpiperidin-3-yl]pyrido[3,4-d]pyridazin-4-amine;
3-methyl-4-(4-{[(3R)-1-methylpiperidin-3-yl]amino}pyrido[3,4-d]pyridazin-1-yl)benzonitrile;
1-(4-chloro-2,6-dimethylphenyl)-N-[(3R)-1-methylpiperidin-3-yl]pyrido[3,4-d]pyridazin-4-amine;
2-(4-{[(3R)-1-cyclopropylpiperidin-3-yl]amino}pyrido[3,4-d]pyridazin-1-yl)-5-(trifluoromethyl)phenol;
2,2,2-trifluoro-1-[3-hydroxy-4-(4-{[(3R)-piperidin-3-yl]amino}phthalazin-1-yl)phenyl]ethan-1-one;
1-(4-chloro-2,6-difluorophenyl)-N-[(3R)-1-methylpiperidin-3-yl]pyrido[3,4-d]pyridazin-4-amine;
1-[2-fluoro-6-methyl-4-(trifluoromethyl)phenyl]-N-[(3R)-1-methylpiperidin-3-yl]pyrido[3,4-d]pyridazin-4-amine;
2-(4-{[(3R)-1-methylpiperidin-3-yl]amino}pyrido[3,4-d]pyridazin-1-yl)-5-nitrophenol;
2-{4-[(1-methylpiperidin-4-yl)amino]pyrido[3,4-d]pyridazin-1-yl}-5-(trifluoromethyl)phenol;
5-(cyclopropylethynyl)-2-(4-{[(3R)-1-methylpiperidin-3-yl]amino}phthalazin-1-yl)phenol;
5-ethynyl-2-(4-{[(3R)-1-methylpiperidin-3-yl]amino}phthalazin-1-yl)phenol;
2-(4-{[(3R)-1-methylpiperidin-3-yl]amino}phthalazin-1-yl)-5-(prop-1-yn-1-yl)phenol;
N-[(3R)-1-methylpiperidin-3-yl]-1-[2-(prop-1-yn-1-yl)-4-(trifluoromethyl)phenyl]pyrido[3,4-d]pyridazin-4-amine;
2-{1-[(5,6,7,8-tetrahydroimidazo[1,2-a]pyridin-6-yl)amino]pyrido[3,4-d]pyridazin-4-yl}-5-(trifluoromethyl)phenol;
2-{1-[(1,4-oxazepan-6-yl)amino]pyrido[3,4-d]pyridazin-4-yl}-5-(trifluoromethyl)phenol;
4-[2-(difluoromethoxy)-4-(trifluoromethyl)phenyl]-N-[(3R)-piperidin-3-yl]pyrido[3,4-d]pyridazin-1-amine;
N-[(3R)-1-methylpiperidin-3-yl]-1-[2-(1-methyl-1H-pyrazol-3-yl)-4-(trifluoromethyl)phenyl]pyrido[3,4-d]pyridazin-4-amine;
2-{4-[3-(dimethylamino)piperidin-1-yl]pyrido[3,4-d]pyridazin-1-yl}-5-(trifluoromethyl)phenol;
1-[4-(difluoromethyl)-2-methoxyphenyl]-N-[(3R)-1-methylpiperidin-3-yl]pyrido[3,4-d]pyridazin-4-amine;
4-[2-(difluoromethoxy)-4-(trifluoromethyl)phenyl]-N-[(3R)-1-ethylpiperidin-3-yl]pyrido[3,4-d]pyridazin-1-amine;
4-[2-(difluoromethoxy)-4-(trifluoromethyl)phenyl]-N-[(3R)-1-methylpiperidin-3-yl]-6,7-dihydro-5H-cyclopenta[d]pyridazin-1-amine;
5-(cyclopropyloxy)-2-(4-{[(3R)-1-methylpiperidin-3-yl]amino}pyrido[3,4-d]pyridazin-1-yl)phenol;
2-[(3R)-3-({4-[2-(difluoromethoxy)-4-(trifluoromethyl)phenyl]pyrido[3,4-d]pyridazin-1-yl}amino)piperidin-1-yl]ethan-1-ol;
1-[4-chloro-2-(difluoromethyl)phenyl]-N-[(3R)-1-methylpiperidin-3-yl]pyrido[3,4-d]pyridazin-4-amine;
N-[(3R)-1-methylpiperidin-3-yl]-1-[2-(pyridin-3-yl)-4-(trifluoromethyl)phenyl]pyrido[3,4-d]pyridazin-4-amine;
4-[4-chloro-2-(difluoromethoxy)phenyl]-N-[(3R)-1-methylpiperidin-3-yl]-6,7-dihydro-5H-cyclopenta[d]pyridazin-1-amine;
1-(2-cyclopropyl-4-methoxyphenyl)-N-[(3R)-1-methylpiperidin-3-yl]pyrido[3,4-d]pyridazin-4-amine;
2-(4-{[(1-methylpiperidin-2-yl)methyl]amino}pyrido[3,4-d]pyridazin-1-yl)-5-(trifluoromethyl)phenol;
4-[2-(difluoromethoxy)-4-methylphenyl]-N-[(3R)-1-methylpiperidin-3-yl]-6,7-dihydro-5H-cyclopenta[d]pyridazin-1-amine;
4-[2-(difluoromethoxy)phenyl]-N-[(3R)-1-methylpiperidin-3-yl]-6,7-dihydro-5H-cyclopenta[d]pyridazin-1-amine;
4-[4-cyclopropyl-2-(difluoromethoxy)phenyl]-N-[(3R)-1-methylpiperidin-3-yl]-6,7-dihydro-5H-cyclopenta[d]pyridazin-1-amine;
1-[4-cyclopropyl-2-(difluoromethoxy)phenyl]-N-[(3R)-1-methylpiperidin-3-yl]pyrido[3,4-d]pyridazin-4-amine;
1-[4-cyclopropyl-2-(difluoromethyl)phenyl]-N-[(3R)-1-methylpiperidin-3-yl]pyrido[3,4-d]pyridazin-4-amine;
1-[4-(difluoromethyl)-2-fluorophenyl]-N-[(3R)-1-methylpiperidin-3-yl]pyrido[3,4-d]pyridazin-4-amine;
2-(4-{[(3R)-1-methylpiperidin-3-yl]amino}thieno[3,4-d]pyridazin-1-yl)-5-(trifluoromethyl)phenol;
N-[(3R)-azepan-3-yl]-1-[2-methoxy-4-(trifluoromethyl)phenyl]pyrido[3,4-d]pyridazin-4-amine;
2-{4-[(1,4-oxazepan-6-yl)amino]pyrido[3,4-d]pyridazin-1-yl}-5-(trifluoromethyl)phenol;
2-{4-[(azepan-4-yl)amino]pyrido[3,4-d]pyridazin-1-yl}-5-(trifluoromethyl)phenol;
1-(4-methoxy-2-methylphenyl)-N-[(3R)-1-methylpiperidin-3-yl]pyrido[3,4-d]pyridazin-4-amine;
2-(3-methyl-4-{[(3R)-1-methylpiperidin-3-yl]amino}[1,2]oxazolo[4,5-d]pyridazin-7-yl)-5-(trifluoromethyl)phenol;
2-(4-{[(3R)-1-(2-hydroxyethyl)piperidin-3-yl]amino}-3-methyl[1,2]oxazolo[4,5-d]pyridazin-7-yl)-5-(trifluoromethyl)phenol;
2-[4-({(3R)-1-[3-(2,2-difluoroethyl)cyclobutyl]piperidin-3-yl}amino)pyrido[3,4-d]pyridazin-1-yl]-5-(trifluoromethyl)phenol;
4-[4-methoxy-2-(trifluoromethyl)phenyl]-N-[(3R)-1-methylpiperidin-3-yl]-6,7-dihydro-5H-cyclopenta[d]pyridazin-1-amine;
4-[2,4-bis(trifluoromethyl)phenyl]-N-[(3R)-1-methylpiperidin-3-yl]-6,7-dihydro-5H-cyclopenta[d]pyridazin-1-amine;
2-{1-[(4-methyl-1,4-oxazepan-6-yl)amino]pyrido[3,4-d]pyridazin-4-yl}-5-(trifluoromethyl)phenol;
2-(4-{[(3R)-azepan-3-yl]amino}pyrido[3,4-d]pyridazin-1-yl)-5-(trifluoromethyl)phenol;
2-{4-[(4-methyl-1,4-oxazepan-6-yl)amino]pyrido[3,4-d]pyridazin-1-yl}-5-(trifluoromethyl)phenol;
2-{4-[(1-methylazepan-4-yl)amino]pyrido[3,4-d]pyridazin-1-yl}-5-(trifluoromethyl)phenol;
2-{4-[(1,4-dimethyl-1,4-diazepan-6-yl)amino]pyrido[3,4-d]pyridazin-1-yl}-5-(trifluoromethyl)phenol;
2-(4-{[(3R)-5,5-difluoro-1-methylpiperidin-3-yl]amino}pyrido[3,4-d]pyridazin-1-yl)-5-(trifluoromethyl)phenol;
ethyl (2S,5R)-5-({1-[2-hydroxy-4-(trifluoromethyl)phenyl]pyrido[3,4-d]pyridazin-4-yl}amino)piperidine-2-carboxylate;
3-methoxy-5-methyl-2-(4-{[(3R)-1-methylpiperidin-3-yl]amino}pyrido[3,4-d]pyridazin-1-yl)phenol;
5-methyl-2-(4-{[(3R)-1-methylpiperidin-3-yl]amino}pyrido[3,4-d]pyridazin-1-yl)benzene-1,3-diol;
2-(4-{[(1R,3S)-3-hydroxycyclohexyl]amino}pyrido[3,4-d]pyridazin-1-yl)-5-(trifluoromethyl)phenol;
2-(4-{[(1R,3R)-3-hydroxycyclohexyl]amino}pyrido[3,4-d]pyridazin-1-yl)-5-(trifluoromethyl)phenol;
2-(4-{[(3R)-1-(oxetan-3-yl)piperidin-3-yl]amino}pyrido[3,4-d]pyridazin-1-yl)-5-(trifluoromethyl)phenol;
1-(4-cyclopropyl-2-methylphenyl)-N-[(3R)-1-methylpiperidin-3-yl]pyrido[3,4-d]pyridazin-4-amine;
3-fluoro-2-(4-{[(3R)-1-(2-hydroxyethyl)piperidin-3-yl]amino}pyrido[3,4-d]pyridazin-1-yl)-5-methylphenol;
3-cyclopropyl-2-(4-{[(3R)-1-methylpiperidin-3-yl]amino}pyrido[3,4-d]pyridazin-1-yl)-5-(trifluoromethyl)phenol;
2-(4-{[2-(dimethylamino)-2-methylpropyl]amino}pyrido[3,4-d]pyridazin-1-yl)-5-(trifluoromethyl)phenol;
N-{(3R)-1-[2-(difluoromethoxy)ethyl]piperidin-3-yl}-1-[2-methoxy-4-(trifluoromethyl)phenyl]pyrido[3,4-d]pyridazin-4-amine;
2-(4-{[(3R)-1-(2,2-difluoroethyl)piperidin-3-yl]amino}pyrido[3,4-d]pyridazin-1-yl)-5-(trifluoromethyl)phenol;
2-(4-{[(3 S,5S)-5-fluoro-1-methylpiperidin-3-yl]amino}pyrido[3,4-d]pyridazin-1-yl)-5-(trifluoromethyl)phenol;
2-(4-{[(3R)-1-(oxan-4-yl)piperidin-3-yl]amino}pyrido[3,4-d]pyridazin-1-yl)-5-(trifluoromethyl)phenol;
2-(4-{[(3R,5R)-5-fluoro-1-methylpiperidin-3-yl]amino}pyrido[3,4-d]pyridazin-1-yl)-5-(trifluoromethyl)phenol;
2-(4-{[(3R)-1-(oxolan-3-yl)piperidin-3-yl]amino}pyrido[3,4-d]pyridazin-1-yl)-5-(trifluoromethyl)phenol;
5-chloro-3-fluoro-2-(4-{[(3R)-1-(2-hydroxyethyl)piperidin-3-yl]amino}pyrido[3,4-d]pyridazin-1-yl)phenol;
2-(4-{[(3R)-1-(2,2,2-trifluoroethyl)piperidin-3-yl]amino}pyrido[3,4-d]pyridazin-1-yl)-5-(trifluoromethyl)phenol;
5-cyclopropyl-3-fluoro-2-(4-{[(3R)-1-(2-hydroxyethyl)piperidin-3-yl]amino}pyrido[3,4-d]pyridazin-1-yl)phenol;
3-fluoro-2-(4-{[(3R)-1-(2-hydroxyethyl)piperidin-3-yl]amino}pyrido[3,4-d]pyridazin-1-yl)phenol;
2-(4-{[(3R)-1-(2-hydroxy-2-methylpropyl)piperidin-3-yl]amino}pyrido[3,4-d]pyridazin-1-yl)-5-(trifluoromethyl)phenol;
1-[2-(difluoromethyl)-4-methoxyphenyl]-N-[(3R)-1-methylpiperidin-3-yl]pyrido[3,4-d]pyridazin-4-amine;
2-[4-(cyclohexylamino)pyrido[3,4-d]pyridazin-1-yl]-5-(trifluoromethyl)phenol;
1-[4-chloro-2-(trifluoromethyl)phenyl]-N-[(3R)-1-methylpiperidin-3-yl]pyrido[3,4-d]pyridazin-4-amine;
N-[(3R)-1-methylpiperidin-3-yl]-1-[2-methyl-4-(trifluoromethyl)phenyl]pyrido[3,4-d]pyridazin-4-amine;
N-[(3R)-1-methylpiperidin-3-yl]-1-{2-[(propan-2-yl)oxy]-4-(trifluoromethyl)phenyl}pyrido[3,4-d]pyridazin-4-amine;
2-(4-{methyl[(3R)-piperidin-3-yl]amino}pyrido[3,4-d]pyridazin-1-yl)-5-(trifluoromethyl)phenol;
2-[4-({(3R)-1-[2-(difluoromethoxy)ethyl]piperidin-3-yl}amino)pyrido[3,4-d]pyridazin-1-yl]-5-(trifluoromethyl)phenol;
2-(4-{[(3R)-1-(3-fluoropropyl)piperidin-3-yl]amino}pyrido[3,4-d]pyridazin-1-yl)-5-(trifluoromethyl)phenol;
2-(4-{[(3R)-1-(propan-2-yl)piperidin-3-yl]amino}pyrido[3,4-d]pyridazin-1-yl)-5-(trifluoromethyl)phenol;
1-[4-methoxy-2-(propan-2-yl)phenyl]-N-[(3S)-1-methylpiperidin-3-yl]pyrido[3,4-d]pyridazin-4-amine;
2-(4-{methyl[(3R)-1-methylpiperidin-3-yl]amino}pyrido[3,4-d]pyridazin-1-yl)-5-(trifluoromethyl)phenol;
N-[(3R)-1-methylpiperidin-3-yl]-1-[4-methyl-2-(trifluoromethyl)phenyl]pyrido[3,4-d]pyridazin-4-amine;
1-[2-(difluoromethyl)-4-methylphenyl]-N-[(3R)-1-methylpiperidin-3-yl]pyrido[3,4-d]pyridazin-4-amine;
2-(4-{[(3R)-1-(1-²H)cyclobutylpiperidin-3-yl]amino}pyrido[3,4-d]pyridazin-1-yl)-5-(trifluoromethyl)phenol;
2-(4-{[(3 S,4R)-4-fluoropiperidin-3-yl]amino}pyrido[3,4-d]pyridazin-1-yl)-5-(trifluoromethyl)phenol;
2-(4-{[(1-methylpiperidin-4-yl)methyl]amino}pyrido[3,4-d]pyridazin-1-yl)-5-(trifluoromethyl)phenol;
2-[4-({[1-(2-hydroxyethyl)piperidin-4-yl]methyl}amino)pyrido[3,4-d]pyridazin-1-yl]-5-(trifluoromethyl)phenol;
3-fluoro-5-methyl-2-(8-{[(3R)-1-methylpiperidin-3-yl]amino}pyrido[2,3-d]pyridazin-5-yl)phenol;
2-(4-{[(1s,3s)-3-hydroxy-3-methylcyclobutyl]amino}pyrido[3,4-d]pyridazin-1-yl)-5-(trifluoromethyl)phenol;
2-(4-{[(3R,5R)-5-fluoropiperidin-3-yl]amino}pyrido[3,4-d]pyridazin-1-yl)-5-(trifluoromethyl)phenol;
5-ethyl-2-(4-{[(3R)-1-methylpiperidin-3-yl]amino}pyrido[3,4-d]pyridazin-1-yl)phenol;
2-(4-{[(1R,3S)-3-methoxycyclohexyl]amino}pyrido[3,4-d]pyridazin-1-yl)-5-(trifluoromethyl)phenol;
2-(4-{[(3R)-oxan-3-yl]amino}pyrido[3,4-d]pyridazin-1-yl)-5-(trifluoromethyl)phenol;
2-(4-{[(3 S)-oxan-3-yl]amino}pyrido[3,4-d]pyridazin-1-yl)-5-(trifluoromethyl)phenol;
1-[2-(difluoromethoxy)-4-(trifluoromethyl)phenyl]-N-[(3R)-piperidin-3-yl]pyrido[3,4-d]pyridazin-4-amine;
N-[(3R)-1-cyclobutylpiperidin-3-yl]-1-[2-(difluoromethoxy)-4-(trifluoromethyl)phenyl]pyrido[3,4-d]pyridazin-4-amine;
2-(7-{[(3R)-1-methylpiperidin-3-yl]amino}furo[2,3-d]pyridazin-4-yl)-5-(trifluoromethyl)phenol;
5-chloro-2-(4-{[(3R)-1-methylpiperidin-3-yl]amino}-6,7-dihydro-5H-cyclopenta[d]pyridazin-1-yl)phenol;
2-(4-{[(3 S,4R)-4-fluoro-1-methylpiperidin-3-yl]amino}pyrido[3,4-d]pyridazin-1-yl)-5-(trifluoromethyl)phenol;
2-(4-{[(1r,3r)-3-hydroxy-3-methylcyclobutyl]amino}pyrido[3,4-d]pyridazin-1-yl)-5-(trifluoromethyl)phenol;
2-(4-{[(3R)-1-(2-hydroxyethyl)azepan-3-yl]amino}pyrido[3,4-d]pyridazin-1-yl)-5-(trifluoromethyl)phenol;
ethyl (2S,5R)-5-({1-[2-hydroxy-4-(trifluoromethyl)phenyl]pyrido[3,4-d]pyridazin-4-yl}amino)-1-methylpiperidine-2-carboxylate;
1-[2-(difluoromethyl)-4-(trifluoromethoxy)phenyl]-N-[(3R)-1-methylpiperidin-3-yl]pyrido[3,4-d]pyridazin-4-amine;
1-[2-(difluoromethyl)-5-(trifluoromethoxy)phenyl]-N-[(3R)-1-methylpiperidin-3-yl]pyrido[3,4-d]pyridazin-4-amine;
2-(8-methyl-4-{[(3R)-1-methylpiperidin-3-yl]amino}pyrrolo[1,2-d][1,2,4]triazin-1-yl)-5-(trifluoromethyl)phenol;
1-[2-(difluoromethoxy)-4-(trifluoromethyl)phenyl]-N-[(3R)-1-methylpiperidin-3-yl]pyrrolo[1,2-d][1,2,4]triazin-4-amine;
N-{(3R)-1-[2-(difluoromethoxy)ethyl]piperidin-3-yl}-1-[2-(difluoromethoxy)-4-(trifluoromethyl)phenyl]pyrrolo[1,2-d][1,2,4]triazin-4-amine;
N-[(3R)-azocan-3-yl]-1-[2-(difluoromethoxy)-4-(trifluoromethyl)phenyl]pyrrolo[1,2-d][1,2,4]triazin-4-amine;
2-[(3R)-3-({1-[2-(difluoromethoxy)-4-(trifluoromethyl)phenyl]pyrrolo[1,2-d][1,2,4]triazin-4-yl}amino)piperidin-1-yl]ethan-1-ol;
1-[2-(difluoromethoxy)-4-(trifluoromethyl)phenyl]-N-[(3R)-1-(oxan-4-yl)piperidin-3-yl]pyrrolo[1,2-d][1,2,4]triazin-4-amine;
5-cyclopropyl-2-(2-methyl-7-{[(3R)-1-methylpiperidin-3-yl]amino}pyrazolo[1,5-d][1,2,4]triazin-4-yl)phenol;
5-methoxy-2-(2-methyl-7-{[(3R)-1-methylpiperidin-3-yl]amino}pyrazolo[1,5-d][1,2,4]triazin-4-yl)phenol;
2-(2-methyl-7-{[(3R)-1-methylpiperidin-3-yl]amino}pyrazolo[1,5-d][1,2,4]triazin-4-yl)-5-(trifluoromethyl)phenol;
4-[2-(difluoromethoxy)-4-(trifluoromethyl)phenyl]-2-methyl-N-[(3R)-1-methylpiperidin-3-yl]pyrazolo[1,5-d][1,2,4]triazin-7-amine;
5-cyclopropyl-2-(4-{[(3R)-1-methylpiperidin-3-yl]amino}pyrrolo[1,2-d][1,2,4]triazin-1-yl)phenol;
2-(2-methyl-4-{[(3R)-piperidin-3-yl]amino}pyrazolo[1,5-d][1,2,4]triazin-7-yl)-5-(trifluoromethyl)phenol;
2-(4-{[(3R)-1-methylpiperidin-3-yl]amino}imidazo[1,5-d][1,2,4]triazin-1-yl)-5-(trifluoromethyl)phenol;
5-bromo-2-(2-methyl-7-{[(3R)-1-methylpiperidin-3-yl]amino}pyrazolo[1,5-d][1,2,4]triazin-4-yl)phenol;
1-[2-(difluoromethoxy)-4-(trifluoromethyl)phenyl]-N-[(3R)-1-ethylpiperidin-3-yl]pyrrolo[1,2-d][1,2,4]triazin-4-amine;
2-(2-methyl-4-{[(3R)-1-methylpiperidin-3-yl]amino}pyrazolo[1,5-d][1,2,4]triazin-7-yl)-5-(trifluoromethyl)phenol;
5-methyl-2-(4-{[(3R)-1-methylpiperidin-3-yl]amino}pyrrolo[1,2-d][1,2,4]triazin-1-yl)phenol;
2-(4-{[(3R)-1-(2-hydroxyethyl)piperidin-3-yl]amino}-2-methylpyrazolo[1,5-d][1,2,4]triazin-7-yl)-5-(trifluoromethyl)phenol;
5-methoxy-2-(2-methyl-4-{[(3R)-piperidin-3-yl]amino}pyrazolo[1,5-d][1,2,4]triazin-7-yl)phenol;
5-cyclopropyl-2-(4-{[(3R)-1-(oxan-4-yl)piperidin-3-yl]amino}pyrrolo[1,2-d][1,2,4]triazin-1-yl)phenol;
3-fluoro-2-(4-{[(3R)-1-(2-hydroxyethyl)piperidin-3-yl]amino}pyrrolo[1,2-d][1,2,4]triazin-1-yl)-5-methylphenol;
3-fluoro-5-methyl-2-(4-{[(3R)-1-methylpiperidin-3-yl]amino}pyrrolo[1,2-d][1,2,4]triazin-1-yl)phenol;
2-(4-{[(3R)-1-(oxan-4-yl)piperidin-3-yl]amino}pyrrolo[1,2-d][1,2,4]triazin-1-yl)-5-(trifluoromethyl)phenol;
2-[4-({(3R)-1-[2-(difluoromethoxy)ethyl]piperidin-3-yl}amino)pyrrolo[1,2-d][1,2,4]triazin-1-yl]-3-fluoro-5-methylphenol;
2-(4-{[(6S)-4-methyl-4-azaspiro[2.5]octan-6-yl]amino}pyrido[3,4-d]pyridazin-1-yl)-5-(trifluoromethyl)phenol;
2-(2-cyclopropyl-7-{[(3R)-1-methylpiperidin-3-yl]amino}pyrazolo[1,5-d][1,2,4]triazin-4-yl)-5-(trifluoromethyl)phenol;
2-(2-cyclopropyl-7-{[(3R)-1-methylpiperidin-3-yl]amino}pyrazolo[1,5-d][1,2,4]triazin-4-yl)-5-methoxyphenol;
1-[2,4-bis(trifluoromethyl)phenyl]-N-[(3R)-1-methylpiperidin-3-yl]pyrrolo[1,2-d][1,2,4]triazin-4-amine;
1-[2-methoxy-4-(trifluoromethyl)phenyl]-N-[(3R)-1-methylpiperidin-3-yl]imidazo[1,5-d][1,2,4]triazin-4-amine;
2-(4-{[(3R)-1-(2-hydroxyethyl)piperidin-3-yl]amino}imidazo[1,5-d][1,2,4]triazin-1-yl)-5-(trifluoromethyl)phenol;
5-methoxy-2-(2-methyl-4-{[(3R)-1-methylpiperidin-3-yl]amino}pyrazolo[1,5-d][1,2,4]triazin-7-yl)phenol;
2-(4-{[(3R)-piperidin-3-yl]amino}pyrrolo[1,2-d][1,2,4]triazin-1-yl)-5-(trifluoromethyl)phenol;
2-(4-{[(3R)-1-(2-hydroxyethyl)piperidin-3-yl]amino}pyrrolo[1,2-d][1,2,4]triazin-1-yl)-5-(trifluoromethyl)phenol;
2-(4-{[(3R)-1-(2-hydroxyethyl)piperidin-3-yl]amino}pyrrolo[1,2-d][1,2,4]triazin-1-yl)-5-methylphenol;
5-chloro-2-(4-{[(3R)-1-methylpiperidin-3-yl]amino}pyrrolo[1,2-d][1,2,4]triazin-1-yl)phenol;
1-[4-chloro-2-(difluoromethoxy)phenyl]-N-[(3R)-1-methylpiperidin-3-yl]pyrrolo[1,2-d][1,2,4]triazin-4-amine;
5-methoxy-2-(4-{[(3R)-1-methylpiperidin-3-yl]amino}pyrrolo[1,2-d][1,2,4]triazin-1-yl)phenol;
2-(4-{[(3R)-1-(2-hydroxyethyl)piperidin-3-yl]amino}pyrrolo[1,2-d][1,2,4]triazin-1-yl)-5-methoxyphenol;
3-fluoro-5-methyl-2-(2-methyl-7-{[(3R)-1-methylpiperidin-3-yl]amino}pyrazolo[1,5-d][1,2,4]triazin-4-yl)phenol;
2-(4-{[(3R)-1-ethylpiperidin-3-yl]amino}imidazo[1,5-d][1,2,4]triazin-1-yl)-5-(trifluoromethyl)phenol;
(1S,3R)-3-({1-[2-(difluoromethoxy)-4-(trifluoromethyl)phenyl]pyrrolo[1,2-d][1,2,4]triazin-4-yl}amino)cyclohexan-1-ol;
1-[2-(difluoromethoxy)-4-(trifluoromethyl)phenyl]-N-[(1R,3S)-3-methoxycyclohexyl]pyrrolo[1,2-d][1,2,4]triazin-4-amine;
3-fluoro-2-(2-methyl-7-{[(3R)-1-methylpiperidin-3-yl]amino}pyrazolo[1,5-d][1,2,4]triazin-4-yl)-5-(trifluoromethyl)phenol;
2-(4-{[(3R)-1-(2-hydroxy-2-methylpropyl)piperidin-3-yl]amino}pyrrolo[1,2-d][1,2,4]triazin-1-yl)-5-(trifluoromethyl)phenol;
2-(4-{[(3R)-piperidin-3-yl]amino}pyrrolo[1,2-d][1,2,4]triazin-1-yl)-5-(trifluoromethoxy)phenol;
2-(4-{[(3R)-1-methylpiperidin-3-yl]amino}pyrrolo[1,2-d][1,2,4]triazin-1-yl)-5-(trifluoromethoxy)phenol;
2-(4-{[(3R)-1-(2-hydroxy-2-methylpropyl)piperidin-3-yl]amino}pyrrolo[1,2-d][1,2,4]triazin-1-yl)-5-methylphenol;
5-chloro-2-(4-{[(3R)-1-(2,2-difluoroethyl)piperidin-3-yl]amino}pyrrolo[1,2-d][1,2,4]triazin-1-yl)phenol;
5-chloro-2-(4-{[(3R)-1-(2-hydroxyethyl)piperidin-3-yl]amino}pyrrolo[1,2-d][1,2,4]triazin-1-yl)phenol;
1-[4-methoxy-2-(trifluoromethyl)phenyl]-N-[(3R)-1-methylpiperidin-3-yl]pyrrolo[1,2-d][1,2,4]triazin-4-amine;
1-[2,4-bis(trifluoromethyl)phenyl]-N-[(3R)-1-ethylpiperidin-3-yl]pyrrolo[1,2-d][1,2,4]triazin-4-amine;
3-fluoro-2-(4-{[(3R)-1-methylpiperidin-3-yl]amino}pyrrolo[1,2-d][1,2,4]triazin-1-yl)-5-(trifluoromethyl)phenol;
2-(4-{[(3R)-1-(2-hydroxyethyl)piperidin-3-yl]amino}pyrrolo[1,2-d][1,2,4]triazin-1-yl)-5-(trifluoromethoxy)phenol;
4-[4-bromo-2-(trifluoromethoxy)phenyl]-2-methyl-N-[(3R)-1-methylpiperidin-3-yl]pyrazolo[1,5-d][1,2,4]triazin-7-amine;
2-[(3R)-3-({4-[2-(difluoromethoxy)-4-(trifluoromethyl)phenyl]-2-methylpyrazolo[1,5-d][1,2,4]triazin-7-yl}amino)piperidin-1-yl]ethan-1-ol;
1-[2-(difluoromethoxy)-4-(trifluoromethyl)phenyl]-N-[(3R)-oxan-3-yl]pyrrolo[1,2-d][1,2,4]triazin-4-amine;
2-(4-{[(3R)-1-ethylpiperidin-3-yl]amino}pyrrolo[1,2-d][1,2,4]triazin-1-yl)-5-methoxyphenol;
(1R,3S)—Ni-{1-[2-(difluoromethoxy)-4-(trifluoromethyl)phenyl]pyrrolo[1,2-d][1,2,4]triazin-4-yl}cyclohexane-1,3-diamine;
5-cyclopropyl-3-fluoro-2-(4-{[(3R)-1-(2-hydroxyethyl)piperidin-3-yl]amino}pyrrolo[1,2-d][1,2,4]triazin-1-yl)phenol;
5-cyclopropyl-3-fluoro-2-(4-{[(3R)-1-methylpiperidin-3-yl]amino}pyrrolo[1,2-d][1,2,4]triazin-1-yl)phenol;
4-[4-methoxy-2-(trifluoromethyl)phenyl]-2-methyl-N-[(3R)-1-methylpiperidin-3-yl]pyrazolo[1,5-d][1,2,4]triazin-7-amine;
4-[2,4-bis(trifluoromethyl)phenyl]-2-methyl-N-[(3R)-1-methylpiperidin-3-yl]pyrazolo[1,5-d][1,2,4]triazin-7-amine;
1-(4-chloro-2-methoxyphenyl)-N-[(3R)-1-methylpiperidin-3-yl]imidazo[1,5-d][1,2,4]triazin-4-amine;
5-chloro-2-(4-{[(3R)-1-methylpiperidin-3-yl]amino}imidazo[1,5-d][1,2,4]triazin-1-yl)phenol;
(3R,5R)-5-({1-[2-(difluoromethoxy)-4-(trifluoromethyl)phenyl]pyrrolo[1,2-d][1,2,4]triazin-4-yl}amino)-1-methylpiperidin-3-ol;
8-[2-methoxy-4-(trifluoromethyl)phenyl]-N-[(3R)-1-methylpiperidin-3-yl]imidazo[1,2-d][1,2,4]triazin-5-amine;
(3R,5R)-5-{[1-(4-cyclopropyl-2-fluoro-6-hydroxyphenyl)pyrrolo[1,2-d][1,2,4]triazin-4-yl]amino}-1-methylpiperidin-3-ol;
1-[2-(difluoromethoxy)-4-(trifluoromethyl)phenyl]-N-[(3R)-oxolan-3-yl]pyrrolo[1,2-d][1,2,4]triazin-4-amine;
(2S)-1-({1-[2-(difluoromethoxy)-4-(trifluoromethyl)phenyl]pyrrolo[1,2-d][1,2,4]triazin-4-yl}amino)propan-2-ol;
4-(4-bromo-2-methoxyphenyl)-N-[(3R)-piperidin-3-yl]pyrazolo[1,5-d][1,2,4]triazin-7-amine;
5-bromo-2-(7-{[(3R)-piperidin-3-yl]amino}pyrazolo[1,5-d][1,2,4]triazin-4-yl)phenol;
N-[(3R)-1-ethylpiperidin-3-yl]-1-[4-methoxy-2-(trifluoromethyl)phenyl]pyrrolo[1,2-d][1,2,4]triazin-4-amine;
2-(8-fluoro-4-{[(3R)-1-methylpiperidin-3-yl]amino}pyrrolo[1,2-d][1,2,4]triazin-1-yl)-5-(trifluoromethyl)phenol;
1-[2-(difluoromethoxy)-4-(trifluoromethyl)phenyl]-8-fluoro-N-[(3R)-1-methylpiperidin-3-yl]pyrrolo[1,2-d][1,2,4]triazin-4-amine;
3-fluoro-2-(4-{[(3R)-oxan-3-yl]amino}pyrrolo[1,2-d][1,2,4]triazin-1-yl)-5-(trifluoromethyl)phenol;
2-methyl-N-[(3R)-1-methylpiperidin-3-yl]-4-[4-methyl-2-(trifluoromethoxy)phenyl]pyrazolo[1,5-d][1,2,4]triazin-7-amine;
2-(4-{[(3R)-oxan-3-yl]amino}imidazo[1,5-d][1,2,4]triazin-1-yl)-5-(trifluoromethyl)phenol;
2-(5-{[(3R)-1-methylpiperidin-3-yl]amino}imidazo[1,2-d][1,2,4]triazin-8-yl)-5-(trifluoromethyl)phenol;
2-(4-{[(1R,3S)-3-hydroxycyclohexyl]amino}pyrrolo[1,2-d][1,2,4]triazin-1-yl)-5-(trifluoromethyl)phenol;
2-(4-{[(1R,3S)-3-methoxycyclohexyl]amino}pyrrolo[1,2-d][1,2,4]triazin-1-yl)-5-(trifluoromethyl)phenol;
2-(4-{[(3R)-oxan-3-yl]amino}pyrrolo[1,2-d][1,2,4]triazin-1-yl)-5-(trifluoromethyl)phenol;
5-chloro-2-(4-{[(3R)-oxan-3-yl]amino}pyrrolo[1,2-d][1,2,4]triazin-1-yl)phenol;
5-chloro-2-(4-{[(3R)-1-(2-hydroxy-2-methylpropyl)piperidin-3-yl]amino}pyrrolo[1,2-d][1,2,4]triazin-1-yl)phenol;
5-chloro-2-(4-{[(3R)-1-(propan-2-yl)piperidin-3-yl]amino}pyrrolo[1,2-d][1,2,4]triazin-1-yl)phenol;
2-(4-{[(1s,3s)-3-hydroxy-3-(trifluoromethyl)cyclobutyl]amino}pyrrolo[1,2-d][1,2,4]triazin-1-yl)-5-(trifluoromethyl)phenol;
2-(4-{[(1s,3s)-3-hydroxy-3-methylcyclobutyl]amino}pyrrolo[1,2-d][1,2,4]triazin-1-yl)-5-(trifluoromethyl)phenol;
3-methoxy-4-(4-{[(3R)-1-methylpiperidin-3-yl]amino}pyrrolo[1,2-d][1,2,4]triazin-1-yl)benzonitrile;
2-(4-{[(3R,5R)-5-fluoropiperidin-3-yl]amino}pyrrolo[1,2-d][1,2,4]triazin-1-yl)-5-(trifluoromethoxy)phenol;
2-(4-{[(3R,5R)-5-fluoro-1-methylpiperidin-3-yl]amino}pyrrolo[1,2-d][1,2,4]triazin-1-yl)-5-(trifluoromethoxy)phenol;
2-(4-{[(3R,5R)-5-fluoro-1-methylpiperidin-3-yl]amino}pyrrolo[1,2-d][1,2,4]triazin-1-yl)-5-(trifluoromethyl)phenol;
2-(4-{[(3R)-1-(2,2-difluoroethyl)piperidin-3-yl]amino}pyrrolo[1,2-d][1,2,4]triazin-1-yl)-5-(trifluoromethyl)phenol;
1-[2-(difluoromethoxy)-4-(trifluoromethyl)phenyl]-N-[(3R,5R)-5-fluoro-1-methylpiperidin-3-yl]pyrrolo[1,2-d][1,2,4]triazin-4-amine;
2-(4-{[(3R)-1-ethylpiperidin-3-yl]amino}pyrrolo[1,2-d][1,2,4]triazin-1-yl)-3-fluoro-5-(trifluoromethyl)phenol;
3-fluoro-2-(4-{[(3R)-1-(2-hydroxyethyl)piperidin-3-yl]amino}pyrrolo[1,2-d][1,2,4]triazin-1-yl)-5-(trifluoromethyl)phenol
3-fluoro-2-(4-{[(3R,5R)-5-fluoro-1-methylpiperidin-3-yl]amino}pyrrolo[1,2-d][1,2,4]triazin-1-yl)-5-(trifluoromethyl)phenol;
3-fluoro-2-(4-{[(3R)-1-(2-hydroxy-2-methylpropyl)piperidin-3-yl]amino}pyrrolo[1,2-d][1,2,4]triazin-1-yl)-5-(trifluoromethyl)phenol;
4-[4-cyclopropyl-2-(trifluoromethoxy)phenyl]-2-methyl-N-[(3R)-1-methylpiperidin-3-yl]pyrazolo[1,5-d][1,2,4]triazin-7-amine;
2-methyl-N-[(3R)-1-methylpiperidin-3-yl]-4-[2-(trifluoromethoxy)phenyl]pyrazolo[1,5-d][1,2,4]triazin-7-amine;
3-fluoro-2-(4-{[(1s,3s)-3-hydroxy-3-methylcyclobutyl]amino}pyrrolo[1,2-d][1,2,4]triazin-1-yl)-5-(trifluoromethyl)phenol;
3-fluoro-5-methyl-2-(4-{[(3R)-oxan-3-yl]amino}pyrrolo[1,2-d][1,2,4]triazin-1-yl)phenol;
3-fluoro-2-(4-{[(1R,3S)-3-hydroxycyclohexyl]amino}pyrrolo[1,2-d][1,2,4]triazin-1-yl)-5-methylphenol;
5-cyclopropyl-3-fluoro-2-(4-{[(3R)-oxan-3-yl]amino}pyrrolo[1,2-d][1,2,4]triazin-1-yl)phenol;
5-cyclopropyl-3-fluoro-2-(4-{[(1R,3S)-3-hydroxycyclohexyl]amino}pyrrolo[1,2-d][1,2,4]triazin-1-yl)phenol;
1-[2-(difluoromethyl)-4-methylphenyl]-N-[(3R)-1-methylpiperidin-3-yl]pyrrolo[1,2-d][1,2,4]triazin-4-amine;
1-[2-(difluoromethyl)-4-(trifluoromethyl)phenyl]-N-[(3R)-1-methylpiperidin-3-yl]pyrrolo[1,2-d][1,2,4]triazin-4-amine;
(1s,3s)-3-({1-[2-(difluoromethyl)-4-(trifluoromethyl)phenyl]pyrrolo[1,2-d][1,2,4]triazin-4-yl}amino)-1-methylcyclobutan-1-ol;
1-[2-(difluoromethoxy)-6-fluoro-4-methylphenyl]-N-[(3R)-1-methylpiperidin-3-yl]pyrrolo[1,2-d][1,2,4]triazin-4-amine;
5-cyclopropyl-3-fluoro-2-(2-methyl-7-{[(3R)-1-methylpiperidin-3-yl]amino}pyrazolo[1,5-d][1,2,4]triazin-4-yl)phenol;
3-fluoro-2-(4-{[(3R)-1-(oxan-4-yl)piperidin-3-yl]amino}pyrrolo[1,2-d][1,2,4]triazin-1-yl)-5-(trifluoromethyl)phenol;
3-methyl-2-(4-{[(3R)-1-methylpiperidin-3-yl]amino}pyrrolo[1,2-d][1,2,4]triazin-1-yl)-5-(trifluoromethyl)phenol;
5-chloro-3-fluoro-2-(4-{[(3R)-1-methylpiperidin-3-yl]amino}pyrrolo[1,2-d][1,2,4]triazin-1-yl)phenol;
5-chloro-2-(4-{[(3R)-1-ethylpiperidin-3-yl]amino}pyrrolo[1,2-d][1,2,4]triazin-1-yl)-3-fluorophenol;
5-chloro-3-fluoro-2-(4-{[(1s,3s)-3-hydroxy-3-methylcyclobutyl]amino}pyrrolo[1,2-d][1,2,4]triazin-1-yl)phenol;
5-chloro-2-(4-{[(3R)-piperidin-3-yl]amino}imidazo[1,5-d][1,2,4]triazin-1-yl)phenol;
5-chloro-2-(4-{[(3R)-1-(2-hydroxyethyl)piperidin-3-yl]amino}imidazo[1,5-d][1,2,4]triazin-1-yl)phenol;
2-(4-{[(2R)-2-hydroxypropyl]amino}pyrrolo[1,2-d][1,2,4]triazin-1-yl)-5-(trifluoromethoxy)phenol;
2-{4-[(2-hydroxy-2-methylpropyl)amino]pyrrolo[1,2-d][1,2,4]triazin-1-yl}-5-(trifluoromethoxy)phenol;
1-[2-(difluoromethoxy)-4-(trifluoromethyl)phenyl]-8-fluoro-N-[(3R,5R)-5-fluoro-1-methylpiperidin-3-yl]pyrrolo[1,2-d][1,2,4]triazin-4-amine;
(2R)-1-({1-[2-(difluoromethoxy)-4-(trifluoromethyl)phenyl]-8-fluoropyrrolo[1,2-d][1,2,4]triazin-4-yl}amino)propan-2-ol;
2-(8-fluoro-4-{[(3R)-1-methylpiperidin-3-yl]amino}pyrrolo[1,2-d][1,2,4]triazin-1-yl)-5-(trifluoromethoxy)phenol;
5-chloro-2-(4-{[(3R)-1-ethylpiperidin-3-yl]amino}imidazo[1,5-d][1,2,4]triazin-1-yl)phenol;
1-[4-chloro-2-(difluoromethyl)phenyl]-N-[(3R)-1-methylpiperidin-3-yl]pyrrolo[1,2-d][1,2,4]triazin-4-amine;
3-fluoro-2-(4-{[(3R,5R)-5-fluoro-1-methylpiperidin-3-yl]amino}pyrrolo[1,2-d][1,2,4]triazin-1-yl)-5-methylphenol;
5-cyclopropyl-3-fluoro-2-(4-{[(3R,5R)-5-fluoro-1-methylpiperidin-3-yl]amino}pyrrolo[1,2-d][1,2,4]triazin-1-yl)phenol;
1-[4-cyclopropyl-2-(difluoromethyl)phenyl]-N-[(3R)-1-methylpiperidin-3-yl]pyrrolo[1,2-d][1,2,4]triazin-4-amine;
3-fluoro-2-(4-{[(3R)-1-(oxolan-3-yl)piperidin-3-yl]amino}pyrrolo[1,2-d][1,2,4]triazin-1-yl)-5-(trifluoromethyl)phenol;
2-(4-{[(1R,3S)-3-hydroxycyclopentyl]amino}pyrrolo[1,2-d][1,2,4]triazin-1-yl)-5-(trifluoromethyl)phenol;
2-(4-{[(1R,3R)-3-hydroxycyclopentyl]amino}pyrrolo[1,2-d][1,2,4]triazin-1-yl)-5-(trifluoromethyl)phenol;
2-{4-[(3-hydroxy-3-methylcyclohexyl)amino]pyrrolo[1,2-d][1,2,4]triazin-1-yl}-5-(trifluoromethyl)phenol;
4-(4-bromo-2-methoxyphenyl)-N-[(3R)-1-methylpiperidin-3-yl]pyrazolo[1,5-d][1,2,4]triazin-7-amine;
5-bromo-2-(7-{[(3R)-1-methylpiperidin-3-yl]amino}pyrazolo[1,5-d][1,2,4]triazin-4-yl)phenol;
5-bromo-2-(7-{[(3R)-1-ethylpiperidin-3-yl]amino}pyrazolo[1,5-d][1,2,4]triazin-4-yl)phenol;
2-[(3R)-3-({1-[4-methoxy-2-(trifluoromethyl)phenyl]pyrrolo[1,2-d][1,2,4]triazin-4-yl}amino)piperidin-1-yl]ethan-1-ol;
5-chloro-3-fluoro-2-(2-methyl-7-{[(3R)-1-methylpiperidin-3-yl]amino}pyrazolo[1,5-d][1,2,4]triazin-4-yl)phenol;
3-fluoro-5-methoxy-2-(4-{[(3R)-1-methylpiperidin-3-yl]amino}pyrrolo[1,2-d][1,2,4]triazin-1-yl)phenol;
3-fluoro-2-(4-{[(3R)-1-(2-hydroxyethyl)piperidin-3-yl]amino}pyrrolo[1,2-d][1,2,4]triazin-1-yl)-5-methoxyphenol;
1-[2-(difluoromethoxy)-4-(trifluoromethyl)phenyl]-N-[(3S)-oxolan-3-yl]pyrrolo[1,2-d][1,2,4]triazin-4-amine;
2-(4-{[(3 S)-oxolan-3-yl]amino}pyrrolo[1,2-d][1,2,4]triazin-1-yl)-5-(trifluoromethyl)phenol;
2-(4-{[(3R)-1-methyl-1,2,3,6-tetrahydropyridin-3-yl]amino}imidazo[1,5-d][1,2,4]triazin-1-yl)-5-(trifluoromethyl)phenol;
2-(7-{[(3R)-1-methylpiperidin-3-yl]amino}[1,2,3]triazolo[1,5-d][1,2,4]triazin-4-yl)-5-(trifluoromethyl)phenol;
1-[2-(difluoromethoxy)-6-fluoro-4-methylphenyl]-N-[(3R,5R)-5-fluoro-1-methylpiperidin-3-yl]pyrrolo[1,2-d][1,2,4]triazin-4-amine;
2-[(3R)-3-({1-[2-(difluoromethoxy)-6-fluoro-4-methylphenyl]pyrrolo[1,2-d][1,2,4]triazin-4-yl}amino)piperidin-1-yl]ethan-1-ol;
1-[2-(difluoromethoxy)-6-fluoro-4-methylphenyl]-N-[(3R)-oxan-3-yl]pyrrolo[1,2-d][1,2,4]triazin-4-amine;
(1s,3s)-3-({1-[2-(difluoromethoxy)-6-fluoro-4-methylphenyl]pyrrolo[1,2-d][1,2,4]triazin-4-yl}amino)-1-methylcyclobutan-1-ol;
3-fluoro-2-(4-{[(1s,3s)-3-hydroxy-3-methylcyclobutyl]amino}pyrrolo[1,2-d][1,2,4]triazin-1-yl)-5-methylphenol;
5-cyclopropyl-3-fluoro-2-(4-{[(1s,3s)-3-hydroxy-3-methylcyclobutyl]amino}pyrrolo[1,2-d][1,2,4]triazin-1-yl)phenol;
1-[2,4-bis(trifluoromethyl)phenyl]-N-[(3R,5R)-5-fluoro-1-methylpiperidin-3-yl]pyrrolo[1,2-d][1,2,4]triazin-4-amine;
1-[2,4-bis(trifluoromethyl)phenyl]-N-{(3R)-1-[2-(difluoromethoxy)ethyl]piperidin-3-yl}pyrrolo[1,2-d][1,2,4]triazin-4-amine;
1-[(3R)-3-({1-[2,4-bis(trifluoromethyl)phenyl]pyrrolo[1,2-d][1,2,4]triazin-4-yl}amino)piperidin-1-yl]-2-methylpropan-2-ol;
1-[2-(difluoromethoxy)-4-methoxyphenyl]-N-[(3R)-1-methylpiperidin-3-yl]pyrrolo[1,2-d][1,2,4]triazin-4-amine;
(1r,3r)-3-({1-[2-(difluoromethoxy)-4-methoxyphenyl]pyrrolo[1,2-d][1,2,4]triazin-4-yl}amino)-1-methylcyclobutan-1-ol;
2-(4-{[(2S)-2-hydroxypropyl]amino}pyrrolo[1,2-d][1,2,4]triazin-1-yl)-5-(trifluoromethyl)phenol;
2-(4-{[(3R)-oxolan-3-yl]amino}pyrrolo[1,2-d][1,2,4]triazin-1-yl)-5-(trifluoromethyl)phenol;
1-[2-(difluoromethoxy)-6-fluoro-4-methylphenyl]-N-[(3R)-1-(oxan-4-yl)piperidin-3-yl]pyrrolo[1,2-d][1,2,4]triazin-4-amine;
1-[(3R)-3-({1-[2-(difluoromethoxy)-6-fluoro-4-methylphenyl]pyrrolo[1,2-d][1,2,4]triazin-4-yl}amino)piperidin-1-yl]-2-methylpropan-2-ol;
3,5-dimethyl-2-(4-{[(3R)-1-methylpiperidin-3-yl]amino}pyrrolo[1,2-d][1,2,4]triazin-1-yl)phenol;
2-(4-{[(3 S,4S)-4-fluoro-1-methylpiperidin-3-yl]amino}pyrrolo[1,2-d][1,2,4]triazin-1-yl)-5-(trifluoromethyl)phenol;
2-(4-{[(3R)-1-ethylpiperidin-3-yl]amino}pyrrolo[1,2-d][1,2,4]triazin-1-yl)-5-(trifluoromethyl)phenol;
2-(4-{[(3R)-1-(propan-2-yl)piperidin-3-yl]amino}pyrrolo[1,2-d][1,2,4]triazin-1-yl)-5-(trifluoromethyl)phenol;
5-chloro-3-fluoro-2-(4-{[(3R,5R)-5-fluoro-1-methylpiperidin-3-yl]amino}pyrrolo[1,2-d][1,2,4]triazin-1-yl)phenol;
5-chloro-3-fluoro-2-(4-{[(3R)-1-(2-hydroxyethyl)piperidin-3-yl]amino}pyrrolo[1,2-d][1,2,4]triazin-1-yl)phenol;
2-(4-{[(3R)-1-(2,2,2-trifluoroethyl)piperidin-3-yl]amino}pyrrolo[1,2-d][1,2,4]triazin-1-yl)-5-(trifluoromethyl)phenol;
5-bromo-2-(4-{[(3R)-1-methylpiperidin-3-yl]amino}pyrrolo[1,2-d][1,2,4]triazin-1-yl)phenol;
1-[4-chloro-2-(2,2,2-trifluoroethoxy)phenyl]-N-[(3R)-1-methylpiperidin-3-yl]pyrrolo[1,2-d][1,2,4]triazin-4-amine;
2-(4-{[(3 S,4R)-4-fluoro-1-methylpiperidin-3-yl]amino}pyrrolo[1,2-d][1,2,4]triazin-1-yl)-5-(trifluoromethyl)phenol;
1-[2-(difluoromethoxy)-6-fluoro-4-(trifluoromethyl)phenyl]-N-[(3R)-1-methylpiperidin-3-yl]pyrrolo[1,2-d][1,2,4]triazin-4-amine;
1-[2-(difluoromethoxy)-6-fluoro-4-methylphenyl]-N-[(3R)-1-(oxolan-3-yl)piperidin-3-yl]pyrrolo[1,2-d][1,2,4]triazin-4-amine;
3-fluoro-5-methyl-2-(4-{[(3R)-1-(oxolan-3-yl)piperidin-3-yl]amino}pyrrolo[1,2-d][1,2,4]triazin-1-yl)phenol;
5-bromo-2-(4-{[(3R)-piperidin-3-yl]amino}pyrrolo[1,2-d][1,2,4]triazin-1-yl)phenol;
2-[(3R)-3-({1-[2,4-bis(trifluoromethyl)phenyl]pyrrolo[1,2-d][1,2,4]triazin-4-yl}amino)piperidin-1-yl]ethan-1-ol;
(2R)-1-({1-[2,4-bis(trifluoromethyl)phenyl]pyrrolo[1,2-d][1,2,4]triazin-4-yl}amino)propan-2-ol;
1-[2-(difluoromethoxy)-4-(trifluoromethyl)phenyl]-N-[(3R)-1-(propan-2-yl)piperidin-3-yl]pyrrolo[1,2-d][1,2,4]triazin-4-amine;
1-[2-(difluoromethoxy)-4-(trifluoromethyl)phenyl]-N-[(3R)-1-(2,2,2-trifluoroethyl)piperidin-3-yl]pyrrolo[1,2-d][1,2,4]triazin-4-amine;
5-cyclopropyl-2-(4-{[(3R)-1-ethylpiperidin-3-yl]amino}pyrrolo[1,2-d][1,2,4]triazin-1-yl)-3-fluorophenol;
5-cyclopropyl-3-fluoro-2-(4-{[(2R)-2-hydroxypropyl]amino}pyrrolo[1,2-d][1,2,4]triazin-1-yl)phenol;
1-[4-chloro-2-(trifluoromethyl)phenyl]-N-[(3R)-1-methylpiperidin-3-yl]pyrrolo[1,2-d][1,2,4]triazin-4-amine;
2-(4-{[(3R)-1-ethylpiperidin-3-yl]amino}pyrrolo[1,2-d][1,2,4]triazin-1-yl)-3-fluoro-5-methylphenol;
3-fluoro-2-(4-{[(2R)-2-hydroxypropyl]amino}pyrrolo[1,2-d][1,2,4]triazin-1-yl)-5-methylphenol;
5-cyclopropyl-3-fluoro-2-(4-{[(2S)-2-hydroxypropyl]amino)pyrrolo[1,2-d][1,2,4]triazin-1-yl)phenol;
5-[(2H₃)methyloxy]-2-(4-{[(3R)-1-methylpiperidin-3-yl]amino}pyrrolo[1,2-d][1,2,4]triazin-1-yl)phenol;
3-fluoro-2-(4-{[(3R,5R)-5-fluoro-1-methylpiperidin-3-yl]amino}pyrrolo[1,2-d][1,2,4]triazin-1-yl)-5-methoxyphenol;
1-[4-bromo-2-(trifluoromethoxy)phenyl]-N-[(3R)-1-methylpiperidin-3-yl]pyrrolo[1,2-d][1,2,4]triazin-4-amine;
1-[4-chloro-2-(difluoromethyl)phenyl]-N-[(3R)-1-ethylpiperidin-3-yl]pyrrolo[1,2-d][1,2,4]triazin-4-amine;
2-[(3R)-3-({1-[4-chloro-2-(difluoromethyl)phenyl]pyrrolo[1,2-d][1,2,4]triazin-4-yl}amino)piperidin-1-yl]ethan-1-ol;
1-[4-cyclopropyl-2-(difluoromethyl)phenyl]-N-[(3R,5R)-5-fluoro-1-methylpiperidin-3-yl]pyrrolo[1,2-d][1,2,4]triazin-4-amine;
1-[4-cyclopropyl-2-(difluoromethyl)phenyl]-N-[(3R)-1-ethylpiperidin-3-yl]pyrrolo[1,2-d][1,2,4]triazin-4-amine;
2-[(3R)-3-({1-[4-cyclopropyl-2-(difluoromethyl)phenyl]pyrrolo[1,2-d][1,2,4]triazin-4-yl}amino)piperidin-1-yl]ethan-1-ol;
5-cyclopropyl-2-(4-{[(3R)-1-(2,2-difluoroethyl)piperidin-3-yl]amino}pyrrolo[1,2-d][1,2,4]triazin-1-yl)-3-fluorophenol;
2-(4-{[(3R)-1-(2,2-difluoroethyl)piperidin-3-yl]amino}pyrrolo[1,2-d][1,2,4]triazin-1-yl)-3-fluoro-5-methylphenol;
2-(4-{[(1R,3S)-3-hydroxycyclohexyl]amino}imidazo[1,5-d][1,2,4]triazin-1-yl)-5-(trifluoromethyl)phenol;
3-fluoro-5-[(²H₃)methyloxy]-2-(4-{[(3R)-1-methylpiperidin-3-yl]amino}pyrrolo[1,2-d][1,2,4]triazin-1-yl)phenol;
2-(4-{[(3R)-1-ethylpiperidin-3-yl]amino}pyrrolo[1,2-d][1,2,4]triazin-1-yl)-3-fluoro-5-[(²H₃)methyloxy]phenol;
2-(7-{[(3R)-1-ethylpiperidin-3-yl]amino}-2-methylpyrazolo[1,5-d][1,2,4]triazin-4-yl)-3-fluoro-5-methylphenol;
1-[2,4-bis(trifluoromethyl)phenyl]-N-[(3R)-1-(oxan-4-yl)piperidin-3-yl]pyrrolo[1,2-d][1,2,4]triazin-4-amine;
1-[2,4-bis(trifluoromethyl)phenyl]-N-[(3R)-1-(oxolan-3-yl)piperidin-3-yl]pyrrolo[1,2-d][1,2,4]triazin-4-amine;
5-chloro-2-(4-{[(3R)-oxan-3-yl]amino}imidazo[1,5-d][1,2,4]triazin-1-yl)phenol;
1-[2-(difluoromethoxy)-4-(trifluoromethoxy)phenyl]-N-[(3R)-1-methylpiperidin-3-yl]pyrrolo[1,2-d][1,2,4]triazin-4-amine;
2-[(3R)-3-({1-[2-(difluoromethoxy)-4-(trifluoromethoxy)phenyl]pyrrolo[1,2-d][1,2,4]triazin-4-yl}amino)piperidin-1-yl]ethan-1-ol;
1-[2-(difluoromethoxy)-4-(trifluoromethoxy)phenyl]-N-[(3R,5R)-5-fluoro-1-methylpiperidin-3-yl]pyrrolo[1,2-d][1,2,4]triazin-4-amine;
(1s,3s)-3-({1-[2-(difluoromethoxy)-4-(trifluoromethoxy)phenyl]pyrrolo[1,2-d][1,2,4]triazin-4-yl}amino)-1-methylcyclobutan-1-ol;
2-(4-{[(1s,3s)-3-hydroxy-3-methylcyclobutyl]amino}pyrrolo[1,2-d][1,2,4]triazin-1-yl)-5-(trifluoromethoxy)phenol;
(1S,3R)-3-({1-[2-(difluoromethoxy)-4-(trifluoromethyl)phenyl]-8-fluoropyrrolo[1,2-d][1,2,4]triazin-4-yl}amino)cyclohexan-1-ol;
(1s,3s)-3-({1-[2-(difluoromethoxy)-4-(trifluoromethyl)phenyl]-8-fluoropyrrolo[1,2-d][1,2,4]triazin-4-yl}amino)-1-methylcyclobutan-1-ol;
2-(8-fluoro-4-{[(1R,3S)-3-hydroxycyclohexyl]amino}pyrrolo[1,2-d][1,2,4]triazin-1-yl)-5-(trifluoromethyl)phenol;
2-(4-{[(1s,3s)-3-hydroxy-3-methylcyclobutyl]amino}imidazo[1,5-d][1,2,4]triazin-1-yl)-5-(trifluoromethyl)phenol;
2-(4-{[(3R,5R)-5-fluoro-1-methylpiperidin-3-yl]amino}imidazo[1,5-d][1,2,4]triazin-1-yl)-5-(trifluoromethyl)phenol;
5-bromo-2-(4-{[(3R)-1-(2-hydroxyethyl)piperidin-3-yl]amino}pyrrolo[1,2-d][1,2,4]triazin-1-yl)phenol;
1-[2,4-bis(trifluoromethyl)phenyl]-N-[(3R)-1-(propan-2-yl)piperidin-3-yl]pyrrolo[1,2-d][1,2,4]triazin-4-amine;
1-({1-[2,4-bis(trifluoromethyl)phenyl]pyrrolo[1,2-d][1,2,4]triazin-4-yl}amino)-2-methylpropan-2-ol;
(1s,3s)-3-({1-[2,4-bis(trifluoromethyl)phenyl]pyrrolo[1,2-d][1,2,4]triazin-4-yl}amino)-1-methylcyclobutan-1-ol;
N-[(3R)-1-methylpiperidin-3-yl]-1-[4-methyl-2-(trifluoromethyl)phenyl]pyrrolo[1,2-d][1,2,4]triazin-4-amine;
1-[4-cyclopropyl-2-(trifluoromethyl)phenyl]-N-[(3R)-1-methylpiperidin-3-yl]pyrrolo[1,2-d][1,2,4]triazin-4-amine;
1-[2,4-bis(trifluoromethyl)phenyl]-N-[(3R,5R)-1-ethyl-5-fluoropiperidin-3-yl]pyrrolo[1,2-d][1,2,4]triazin-4-amine;
1-({1-[2-(difluoromethoxy)-4-(trifluoromethyl)phenyl]pyrrolo[1,2-d][1,2,4]triazin-4-yl}amino)-2-methylpropan-2-ol;
(1s,3s)-3-({1-[2-(difluoromethoxy)-4-(trifluoromethyl)phenyl]pyrrolo[1,2-d][1,2,4]triazin-4-yl}amino)-1-methylcyclobutan-1-ol;
1-[2-(difluoromethoxy)-4-(trifluoromethyl)phenyl]-N-[(3R,5R)-1-ethyl-5-fluoropiperidin-3-yl]pyrrolo[1,2-d][1,2,4]triazin-4-amine;
3-fluoro-2-{4-[(2-hydroxy-2-methylpropyl)amino]pyrrolo[1,2-d][1,2,4]triazin-1-yl}-5-methylphenol;
5-cyclopropyl-3-fluoro-2-{4-[(2-hydroxy-2-methylpropyl)amino]pyrrolo[1,2-d][1,2,4]triazin-1-yl}phenol;
5-cyclopropyl-3-fluoro-2-(4-{[(3R)-1-(propan-2-yl)piperidin-3-yl]amino}pyrrolo[1,2-d][1,2,4]triazin-1-yl)phenol;
5-cyclopropyl-3-fluoro-2-(4-{[(3R)-1-(2,2,2-trifluoroethyl)piperidin-3-yl]amino}pyrrolo[1,2-d][1,2,4]triazin-1-yl)phenol;
2-(6-methyl-4-{[(3R)-1-methylpiperidin-3-yl]amino}imidazo[1,5-d][1,2,4]triazin-1-yl)-5-(trifluoromethyl)phenol;
5-bromo-2-(7-{[(1s,3s)-3-hydroxy-3-methylcyclobutyl]amino}pyrazolo[1,5-d][1,2,4]triazin-4-yl)phenol;
4-[2-(benzyloxy)-4-bromophenyl]-N-[(3R)-1-methylpiperidin-3-yl]pyrazolo[1,5-d][1,2,4]triazin-7-amine;
5-bromo-2-(7-{[(3R,5R)-5-fluoro-1-methylpiperidin-3-yl]amino}pyrazolo[1,5-d][1,2,4]triazin-4-yl)phenol;
5-bromo-2-(7-{[(3R)-oxan-3-yl]amino}pyrazolo[1,5-d][1,2,4]triazin-4-yl)phenol;
5-bromo-2-(7-{[(3R)-1-(2,2-difluoroethyl)piperidin-3-yl]amino}pyrazolo[1,5-d][1,2,4]triazin-4-yl)phenol;
5-chloro-2-(4-{[(1s,3s)-3-hydroxy-3-methylcyclobutyl]amino}imidazo[1,5-d][1,2,4]triazin-1-yl)phenol;
5-methyl-2-(2-methyl-7-{[(3R)-1-methylpiperidin-3-yl]amino}pyrazolo[1,5-d][1,2,4]triazin-4-yl)phenol;
3-fluoro-5-methoxy-2-(2-methyl-7-{[(3R)-1-methylpiperidin-3-yl]amino}pyrazolo[1,5-d][1,2,4]triazin-4-yl)phenol;
5-bromo-2-(4-{[(3R)-1-(1-hydroxypropan-2-yl)piperidin-3-yl]amino}pyrrolo[1,2-d][1,2,4]triazin-1-yl)phenol;
5-chloro-2-(4-{[(3R)-1-(propan-2-yl)piperidin-3-yl]amino}imidazo[1,5-d][1,2,4]triazin-1-yl)phenol;
5-cyclopropyl-2-(7-{[(3R)-1-ethylpiperidin-3-yl]amino}-2-methylpyrazolo[1,5-d][1,2,4]triazin-4-yl)-3-fluorophenol;
2-(7-{[(3R)-1-ethylpiperidin-3-yl]amino}-2-methylpyrazolo[1,5-d][1,2,4]triazin-4-yl)-3-fluoro-5-methoxyphenol;
4-[2-(difluoromethoxy)-4-(trifluoromethyl)phenyl]-N-[(3R)-1-ethylpiperidin-3-yl]-2-methylpyrazolo[1,5-d][1,2,4]triazin-7-amine;
5-chloro-2-(4-{[(3R,5R)-5-fluoro-1-methylpiperidin-3-yl]amino}imidazo[1,5-d][1,2,4]triazin-1-yl)phenol;
5-chloro-2-(4-{[(3R)-1-(2,2,2-trifluoroethyl)piperidin-3-yl]amino}imidazo[1,5-d][1,2,4]triazin-1-yl)phenol;
5-chloro-2-(4-{[(3R)-1-(2,2-difluoroethyl)piperidin-3-yl]amino}imidazo[1,5-d][1,2,4]triazin-1-yl)phenol;
3-methyl-2-(2-methyl-7-{[(3R)-1-methylpiperidin-3-yl]amino}pyrazolo[1,5-d][1,2,4]triazin-4-yl)-5-(trifluoromethyl)phenol;
5-chloro-2-(4-{[(3R)-oxolan-3-yl]amino}imidazo[1,5-d][1,2,4]triazin-1-yl)phenol;
3-fluoro-2-(4-{[(1s,3s)-3-hydroxy-3-methylcyclobutyl]amino}pyrrolo[1,2-d][1,2,4]triazin-1-yl)-5-methoxyphenol;
5-ethyl-3-fluoro-2-(4-{[(3R)-1-methylpiperidin-3-yl]amino}pyrrolo[1,2-d][1,2,4]triazin-1-yl)phenol;
5-bromo-2-(4-{[(3R)-1-ethylpiperidin-3-yl]amino}pyrrolo[1,2-d][1,2,4]triazin-1-yl)phenol;
1-[2-(difluoromethoxy)-4-(trifluoromethoxy)phenyl]-N-[(3R)-1-ethylpiperidin-3-yl]pyrrolo[1,2-d][1,2,4]triazin-4-amine;
1-[2-(difluoromethoxy)-4-(trifluoromethyl)phenyl]-N-[(3R)-1-ethylpiperidin-3-yl]-8-fluoropyrrolo[1,2-d][1,2,4]triazin-4-amine;
2-{4-[(1-methylpiperidin-3-yl)methyl]pyrido[3,4-d]pyridazin-1-yl}-5-(trifluoromethyl)phenol;
1-[2-(difluoromethoxy)-4-(trifluoromethyl)phenyl]-N-(2-methoxy-2-methylpropyl)pyrrolo[1,2-d][1,2,4]triazin-4-amine;
3-fluoro-5-methyl-2-(4-{[(3R)-1-(propan-2-yl)piperidin-3-yl]amino}pyrrolo[1,2-d][1,2,4]triazin-1-yl)phenol;
3-fluoro-5-methyl-2-(4-{[(3R)-1-(2,2,2-trifluoroethyl)piperidin-3-yl]amino}pyrrolo[1,2-d][1,2,4]triazin-1-yl)phenol;
2-(4-{[(3R,5R)-1-ethyl-5-fluoropiperidin-3-yl]amino}pyrrolo[1,2-d][1,2,4]triazin-1-yl)-3-fluoro-5-methylphenol;
2-(4-{[(3R,5R)-1-ethyl-5-fluoropiperidin-3-yl]amino}pyrrolo[1,2-d][1,2,4]triazin-1-yl)-5-(trifluoromethoxy)phenol;
(1s,3s)-3-({1-[2,4-bis(difluoromethoxy)phenyl]pyrrolo[1,2-d][1,2,4]triazin-4-yl}amino)-1-methylcyclobutan-1-ol;
1-[2-(difluoromethoxy)-4-(trifluoromethyl)phenyl]-N-[(3R)-1-ethylpiperidin-3-yl]imidazo[1,5-d][1,2,4]triazin-4-amine;
2-(4-{[(3R,5S)-5-fluoropiperidin-3-yl]amino}pyrrolo[1,2-d][1,2,4]triazin-1-yl)-5-(trifluoromethyl)phenol;
5-cyclopropyl-2-(4-{[(3R,5R)-1-ethyl-5-fluoropiperidin-3-yl]amino}pyrrolo[1,2-d][1,2,4]triazin-1-yl)-3-fluorophenol;
5-chloro-3-fluoro-2-(4-{[(2S)-2-hydroxypropyl]amino}pyrrolo[1,2-d][1,2,4]triazin-1-yl)phenol;
5-chloro-2-(4-{[(1R,3S)-3-hydroxycyclohexyl]amino}imidazo[1,5-d][1,2,4]triazin-1-yl)phenol;
5-chloro-2-(7-{[(3R)-1-ethylpiperidin-3-yl]amino}-2-methylpyrazolo[1,5-d][1,2,4]triazin-4-yl)-3-fluorophenol;
5-chloro-2-(4-{[(3S)-oxolan-3-yl]amino}imidazo[1,5-d][1,2,4]triazin-1-yl)phenol;
5-chloro-2-(4-{[(1R,3S)-3-methoxycyclohexyl]amino}imidazo[1,5-d][1,2,4]triazin-1-yl)phenol;
2-[4-({[(2S)-1-ethylpyrrolidin-2-yl]methyl}amino)pyrrolo[1,2-d][1,2,4]triazin-1-yl]-3-fluoro-5-methylphenol;
5-cyclopropyl-2-[4-({[(2S)-1-ethylpyrrolidin-2-yl]methyl}amino)pyrrolo[1,2-d][1,2,4]triazin-1-yl]-3-fluorophenol;
2-(7-{[(3R)-1-ethylpiperidin-3-yl]amino}-2-methylpyrazolo[1,5-d][1,2,4]triazin-4-yl)-5-(trifluoromethoxy)phenol;
4-[2,4-bis(trifluoromethyl)phenyl]-N-[(3R)-1-ethylpiperidin-3-yl]-2-methylpyrazolo[1,5-d][1,2,4]triazin-7-amine;
5-cyclopropyl-2-(4-{[(2R)-2-hydroxypropyl]amino}pyrrolo[1,2-d][1,2,4]triazin-1-yl)phenol;
2-(4-{[(2R)-2-hydroxypropyl]amino}pyrrolo[1,2-d][1,2,4]triazin-1-yl)-5-methoxyphenol;
2-(4-{[(2R)-2-hydroxypropyl]amino}pyrrolo[1,2-d][1,2,4]triazin-1-yl)-5-(trifluoromethyl)phenol;
1-[2,4-bis(trifluoromethyl)phenyl]-N-{[(3S)-oxolan-3-yl]methyl}pyrrolo[1,2-d][1,2,4]triazin-4-amine;
1-[2,4-bis(trifluoromethyl)phenyl]-N-{[(3R)-oxolan-3-yl]methyl}pyrrolo[1,2-d][1,2,4]triazin-4-amine;
1-[2-(difluoromethoxy)-4-(trifluoromethyl)phenyl]-7-methyl-N-[(3R)-1-methylpiperidin-3-yl]pyrrolo[1,2-d][1,2,4]triazin-4-amine;
1-[2-(difluoromethoxy)-4-(trifluoromethyl)phenyl]-8-fluoro-N-[(3R)-1-(propan-2-yl)piperidin-3-yl]pyrrolo[1,2-d][1,2,4]triazin-4-amine;
2-(8-fluoro-4-{[(1s,3s)-3-hydroxy-3-methylcyclobutyl]amino}pyrrolo[1,2-d][1,2,4]triazin-1-yl)-5-(trifluoromethyl)phenol;
2-(4-{[(3R)-1-ethylpiperidin-3-yl]amino}-8-fluoropyrrolo[1,2-d][1,2,4]triazin-1-yl)-5-(trifluoromethyl)phenol;
2-(8-fluoro-4-{[(2S)-2-hydroxypropyl]amino}pyrrolo[1,2-d][1,2,4]triazin-1-yl)-5-(trifluoromethyl)phenol;
2-(8-fluoro-4-{[(3R,5R)-5-fluoro-1-methylpiperidin-3-yl]amino}pyrrolo[1,2-d][1,2,4]triazin-1-yl)-5-(trifluoromethyl)phenol;
(3R,5R)-5-({8-fluoro-1-[2-hydroxy-4-(trifluoromethyl)phenyl]pyrrolo[1,2-d][1,2,4]triazin-4-yl}amino)-1-methylpiperidin-3-ol;
2-(4-{[(3R,5R)-1-ethyl-5-fluoropiperidin-3-yl]amino}-8-fluoropyrrolo[1,2-d][1,2,4]triazin-1-yl)-5-(trifluoromethoxy)phenol;
5-cyclopropyl-2-(8-fluoro-4-{[(3R)-1-methylpiperidin-3-yl]amino}pyrrolo[1,2-d][1,2,4]triazin-1-yl)phenol;
5-cyclopropyl-2-(4-{[(3R)-1-ethylpiperidin-3-yl]amino}-8-fluoropyrrolo[1,2-d][1,2,4]triazin-1-yl)phenol;
5-cyclopropyl-3-fluoro-2-(8-fluoro-4-{[(3R)-1-methylpiperidin-3-yl]amino}pyrrolo[1,2-d][1,2,4]triazin-1-yl)phenol;
5-cyclopropyl-2-(4-{[(3R)-1-ethylpiperidin-3-yl]amino}-8-fluoropyrrolo[1,2-d][1,2,4]triazin-1-yl)-3-fluorophenol;
(3R,5R)-5-({1-[2-(difluoromethoxy)-4-(trifluoromethyl)phenyl]-8-fluoropyrrolo[1,2-d][1,2,4]triazin-4-yl}amino)-1-ethylpiperidin-3-ol;
2-(4-{[(3R)-1-ethylpiperidin-3-yl]amino}-8-fluoropyrrolo[1,2-d][1,2,4]triazin-1-yl)-5-(trifluoromethoxy)phenol;
(3R,5R)-1-ethyl-5-({8-fluoro-1-[2-hydroxy-4-(trifluoromethoxy)phenyl]pyrrolo[1,2-d][1,2,4]triazin-4-yl}amino)piperidin-3-ol;
2-(4-{[(3R)-1-ethylpiperidin-3-yl]amino}pyrrolo[1,2-d][1,2,4]triazin-1-yl)-3-methyl-5-(trifluoromethyl)phenol;
3-fluoro-2-(7-methyl-4-{[(3R)-1-methylpiperidin-3-yl]amino}pyrrolo[1,2-d][1,2,4]triazin-1-yl)-5-(trifluoromethyl)phenol;
5-chloro-3-fluoro-2-(4-{[(3R)-oxan-3-yl]amino}pyrrolo[1,2-d][1,2,4]triazin-1-yl)phenol;
2-(4-{[(3R)-1-ethylpiperidin-3-yl]amino}pyrrolo[1,2-d][1,2,4]triazin-1-yl)-5-(trifluoromethoxy)phenol;
1-[2-(difluoromethoxy)-4-(trifluoromethyl)phenyl]-N-{[(2S)-oxolan-2-yl]methyl}pyrrolo[1,2-d][1,2,4]triazin-4-amine;
1-[2-(difluoromethoxy)-4-(trifluoromethyl)phenyl]-N-{[(2R)-oxolan-2-yl]methyl}pyrrolo[1,2-d][1,2,4]triazin-4-amine;
1-[2,4-bis(difluoromethoxy)phenyl]-N-[(3R)-1-methylpiperidin-3-yl]pyrrolo[1,2-d][1,2,4]triazin-4-amine;
1-[2,4-bis(difluoromethoxy)phenyl]-N-[(3R)-1-ethylpiperidin-3-yl]pyrrolo[1,2-d][1,2,4]triazin-4-amine;
2-[(3R)-3-({1-[2,4-bis(difluoromethoxy)phenyl]pyrrolo[1,2-d][1,2,4]triazin-4-yl}amino)piperidin-1-yl]ethan-1-ol;
1-[2,4-bis(difluoromethoxy)phenyl]-N-[(3R,5R)-5-fluoro-1-methylpiperidin-3-yl]pyrrolo[1,2-d][1,2,4]triazin-4-amine;
5-(difluoromethoxy)-2-(4-{[(3R)-1-ethylpiperidin-3-yl]amino}pyrrolo[1,2-d][1,2,4]triazin-1-yl)-3-fluorophenol;
5-(difluoromethoxy)-3-fluoro-2-(4-{[(3R,5R)-5-fluoro-1-methylpiperidin-3-yl]amino}pyrrolo[1,2-d][1,2,4]triazin-1-yl)phenol;
5-(difluoromethoxy)-3-fluoro-2-(4-{[(1s,3s)-3-hydroxy-3-methylcyclobutyl]amino}pyrrolo[1,2-d][1,2,4]triazin-1-yl)phenol;
5-(difluoromethoxy)-2-(4-{[(3R,5R)-1-ethyl-5-fluoropiperidin-3-yl]amino}pyrrolo[1,2-d][1,2,4]triazin-1-yl)-3-fluorophenol;
5-chloro-2-(4-{[(3R,5R)-1-ethyl-5-fluoropiperidin-3-yl]amino}pyrrolo[1,2-d][1,2,4]triazin-1-yl)-3-fluorophenol;
5-chloro-3-fluoro-2-(4-{[(oxolan-2-yl)methyl]amino}pyrrolo[1,2-d][1,2,4]triazin-1-yl)phenol;
5-bromo-2-(4-{[(3R)-1-(2-hydroxyethyl)piperidin-3-yl]amino}imidazo[1,5-d][1,2,4]triazin-1-yl)phenol;
1-[2-(difluoromethoxy)-4-(trifluoromethyl)phenyl]-N-[(3R)-1-methylpiperidin-3-yl]imidazo[1,5-d][1,2,4]triazin-4-amine;
1-[2-(difluoromethoxy)-4-(trifluoromethyl)phenyl]-N-[(3R,5R)-5-fluoro-1-methylpiperidin-3-yl]imidazo[1,5-d][1,2,4]triazin-4-amine;
2-(4-{[(3R)-1-ethylpiperidin-3-yl]amino}imidazo[1,5-d][1,2,4]triazin-1-yl)-3-fluoro-5-methylphenol;
5-bromo-2-(4-{[(1s,3s)-3-hydroxy-3-methylcyclobutyl]amino}imidazo[1,5-d][1,2,4]triazin-1-yl)phenol;
5-bromo-2-(4-{[(3R)-1-ethylpiperidin-3-yl]amino}imidazo[1,5-d][1,2,4]triazin-1-yl)phenol;
5-ethoxy-3-fluoro-2-(4-{[(1s,3s)-3-hydroxy-3-methylcyclobutyl]amino}pyrrolo[1,2-d][1,2,4]triazin-1-yl)phenol;
5-ethoxy-3-fluoro-2-(4-{[(3R)-1-methylpiperidin-3-yl]amino}pyrrolo[1,2-d][1,2,4]triazin-1-yl)phenol;
5-ethoxy-3-fluoro-2-(4-{[(3R,5R)-5-fluoro-1-methylpiperidin-3-yl]amino}pyrrolo[1,2-d][1,2,4]triazin-1-yl)phenol;
2-(7-{[(3R)-1-ethylpiperidin-3-yl]amino}pyrazolo[1,5-d][1,2,4]triazin-4-yl)-5-methoxyphenol;
2-(7-{[(3R)-1-ethylpiperidin-3-yl]amino}pyrazolo[1,5-d][1,2,4]triazin-4-yl)-5-methylphenol;
5-methoxy-2-(7-{[(3R)-1-methylpiperidin-3-yl]amino}pyrazolo[1,5-d][1,2,4]triazin-4-yl)phenol;
(R)-2-(4-((1-ethylpiperidin-3-yl)amino)pyrrolo[1,2-d][1,2,4]triazin-1-yl)-3, 5-dimethylphenol;
(3S,5R)-5-((1-(2-fluoro-6-hydroxy-4-methylphenyl)pyrrolo[1,2-d][1,2,4]triazin-4-yl)amino)-1-methylpiperidin-3-ol;
(3S,5R)-1-ethyl-5-((1-(2-fluoro-6-hydroxy-4-methylphenyl)pyrrolo[1,2-d][1,2,4]triazin-4-yl)amino)piperidin-3-ol;
(3S,5R)-5-((1-(4-cyclopropyl-2-fluoro-6-hydroxyphenyl)pyrrolo[1,2-d][1,2,4]triazin-4-yl)amino)-1-methylpiperidin-3-ol; and
(3S,5R)-5-((1-(4-cyclopropyl-2-fluoro-6-hydroxyphenyl)pyrrolo[1,2-d][1,2,4]triazin-4-yl)amino)-1-ethylpiperidin-3-ol;
   wherein a form of the compound is selected from the group consisting of a pharmaceutically acceptable salt, hydrate, solvate, racemate, enantiomer, diastereomer,
   stereoisomer, and tautomer form thereof.

Another aspect of the invention provides any one of the compounds selected from the following:
2-(4-{[(3R)-1-methylpiperidin-3-yl]amino}phthalazin-1-yl)-5-(trifluoromethyl)phenol formate;
2-(4-{[(3R)-1-methylpiperidin-3-yl]amino}-5,6,7,8-tetrahydrophthalazin-1-yl)-5-(trifluoromethyl)phenol formate;
2-(4-{[(3R)-1-methylpiperidin-3-yl]amino}-6,7-dihydro-5H-cyclopenta[d]pyridazin-1-yl)-5-(trifluoromethyl)phenol formate;
2-{4-[(pyrrolidin-3-yl)amino]phthalazin-1-yl}-5-(trifluoromethyl)phenol formate;
2-(4-{[(3R)-piperidin-3-yl]amino}phthalazin-1-yl)-5-(trifluoromethyl)phenol hydrochloride;
2-(8-methyl-4-{[(3R)-piperidin-3-yl]amino}-5,6,7,8-tetrahydrophthalazin-1-yl)-5-(trifluoromethyl)phenol formate;
2-{4-[(piperidin-3-yl)methyl]phthalazin-1-yl}-5-(trifluoromethyl)phenol hydrochloride;
2-(4-{[(3R)-piperidin-3-yl]amino}pyrido[3,4-d]pyridazin-1-yl)-5-(trifluoromethyl)phenol diformate;
2-(4-{[(3R)-1-ethylpiperidin-3-yl]amino}pyrido[3,4-d]pyridazin-1-yl)-5-(trifluoromethyl)phenol formate;
2-{4-[(1-methylpiperidin-3-yl)methyl]phthalazin-1-yl}-5-(trifluoromethyl)phenol formate;
2-(4-{[(3R)-1-(2-hydroxyethyl)piperidin-3-yl]amino}phthalazin-1-yl)-5-(trifluoromethyl)phenol formate;
(3S,5R)-5-({4-[2-hydroxy-4-(trifluoromethyl)phenyl]phthalazin-1-yl}amino)piperidin-3-ol formate;
2-(1-{[(3R)-1-methylpiperidin-3-yl]amino}pyrido[3,4-d]pyridazin-4-yl)-5-(trifluoromethyl)phenol N-ethylethanamine;
2-(4-{[(3R)-1-(2-hydroxyethyl)piperidin-3-yl]amino}pyrido[3,4-d]pyridazin-1-yl)-5-(trifluoromethyl)phenol diformate;
(3S,5R)-5-({4-[2-hydroxy-4-(trifluoromethyl)phenyl]phthalazin-1-yl}amino)-1-methylpiperidin-3-ol formate;
2-(5-{[(3R)-1-methylpiperidin-3-yl]amino}pyrido[2,3-d]pyridazin-8-yl)-5-(trifluoromethyl)phenol formate;
(3R,5R)-5-({4-[2-hydroxy-4-(trifluoromethyl)phenyl]phthalazin-1-yl}amino)piperidin-3-ol formate;
2-(4-{[(3R)-1-methylpiperidin-3-yl]amino}furo[2,3-d]pyridazin-7-yl)-5-(trifluoromethyl)phenol formate;
5-methyl-2-(8-{[(3R)-1-methylpiperidin-3-yl]amino}pyrido[2,3-d]pyridazin-5-yl)phenol formate;
[(3R)-3-({4-[2-hydroxy-4-(trifluoromethyl)phenyl]phthalazin-1-yl}amino)piperidin-1-yl]acetic acid formate;
2-{1-[(piperidin-3-yl)methyl]pyrido[3,4-d]pyridazin-4-yl}-5-(trifluoromethyl)phenol hydrochloride;
2-(4-{[(3R)-1-methylpiperidin-3-yl]amino}phthalazin-1-yl)-5-(1-methylpyrrolidin-3-yl)phenol formate;
2-[1-({[(2S)-pyrrolidin-2-yl]methyl}amino)pyrido[3,4-d]pyridazin-4-yl]-5-(trifluoromethyl)phenol formate;
2-[4-({[(2S)-pyrrolidin-2-yl]methyl}amino)pyrido[3,4-d]pyridazin-1-yl]-5-(trifluoromethyl)phenol formate;
2-[4-({[(2S)-1-methylpyrrolidin-2-yl]methyl}amino)pyrido[3,4-d]pyridazin-1-yl]-5-(trifluoromethyl)phenol formate;
2-[4-({[(2S)-1-ethylpyrrolidin-2-yl]methyl}amino)pyrido[3,4-d]pyridazin-1-yl]-5-(trifluoromethyl)phenol formate;
2-{1-[(1-methylpiperidin-3-yl)methyl]pyrido[3,4-d]pyridazin-4-yl}-5-(trifluoromethyl)phenol formate;
1-[2-methoxy-4-(trifluoromethyl)phenyl]-N-[(3R)-1-methylpiperidin-3-yl]pyrido[3,4-d]pyridazin-4-amine formate;
4-[2-(difluoromethoxy)-4-(trifluoromethyl)phenyl]-N-[(3R)-1-methylpiperidin-3-yl]phthalazin-1-amine formate;
1-[2-(difluoromethoxy)-4-(trifluoromethyl)phenyl]-N-[(3R)-1-methylpiperidin-3-yl]pyrido[3,4-d]pyridazin-4-amine formate;
4-[2-(difluoromethoxy)-4-(trifluoromethyl)phenyl]-N-[(3R)-1-methylpiperidin-3-yl]pyrido[3,4-d]pyridazin-1-amine formate;
4-[2-amino-4-(trifluoromethyl)phenyl]-N-[(3R)-1-methylpiperidin-3-yl]phthalazin-1-amine formate;
N-[2-(4-{[(3R)-1-methylpiperidin-3-yl]amino}phthalazin-1-yl)-5-(trifluoromethyl)phenyl]methanesulfonamide formate;
4-[2-(methylamino)-4-(trifluoromethyl)phenyl]-N-[(3R)-1-methylpiperidin-3-yl]phthalazin-1-amine formate;
N-[2-(4-{[(3R)-1-methylpiperidin-3-yl]amino}phthalazin-1-yl)-5-(trifluoromethyl)phenyl]acetamide formate;
1-[2-fluoro-4-(trifluoromethyl)phenyl]-N-[(3R)-1-methylpiperidin-3-yl]pyrido[3,4-d]pyridazin-4-amine formate;
1-(4-chloro-2-fluorophenyl)-N-[(3R)-1-methylpiperidin-3-yl]pyrido[3,4-d]pyridazin-4-amine formate;
1-[4-chloro-2-(difluoromethoxy)phenyl]-N-[(3R)-1-methylpiperidin-3-yl]pyrido[3,4-d]pyridazin-4-amine formate;
4-[4-chloro-2-(difluoromethoxy)phenyl]-N-[(3R)-1-methylpiperidin-3-yl]phthalazin-1-amine formate;
1-[2-(difluoromethoxy)-4-methylphenyl]-N-[(3R)-1-methylpiperidin-3-yl]pyrido[3,4-d]pyridazin-4-amine formate;
4-[2-(difluoromethoxy)-4-methylphenyl]-N-[(3R)-1-methylpiperidin-3-yl]phthalazin-1-amine formate;
1-[2-chloro-4-(trifluoromethyl)phenyl]-N-[(3R)-1-methylpiperidin-3-yl]pyrido[3,4-d]pyridazin-4-amine formate;
4-[2-methoxy-4-(trifluoromethyl)phenyl]-1-methyl-N-[(3R)-1-methylpiperidin-3-yl]-1H-pyrazolo[3,4-d]pyridazin-7-amine formate;
2-(1-methyl-7-{[(3R)-1-methylpiperidin-3-yl]amino}-1H-pyrazolo[3,4-d]pyridazin-4-yl)-5-(trifluoromethyl)phenol formate;
2-(1-methyl-8-{[(3R)-1-methylpiperidin-3-yl]amino}-1,2,3,4-tetrahydropyrido[2,3-d]pyridazin-5-yl)-5-(trifluoromethyl)phenol triformate;
5-chloro-2-(4-{[(3R)-1-methylpiperidin-3-yl]amino}pyrido[3,4-d]pyridazin-1-yl)benzonitrile formate;
2-amino-3-chloro-6-(4-{[(3R)-1-methylpiperidin-3-yl]amino}phthalazin-1-yl)phenol formate;
4-(2,3-difluoro-4-methylphenyl)-N-[(3R)-1-methylpiperidin-3-yl]phthalazin-1-amine formate;
5-methoxy-2-(4-{[(3R)-1-methylpiperidin-3-yl]amino}pyrido[3,4-d]pyridazin-1-yl)phenol formate;
1-[2,4-bis(trifluoromethyl)phenyl]-N-[(3R)-1-methylpiperidin-3-yl]pyrido[3,4-d]pyridazin-4-amine formate;
1-[4-methoxy-2-(trifluoromethyl)phenyl]-N-[(3R)-1-methylpiperidin-3-yl]pyrido[3,4-d]pyridazin-4-amine formate;
2-(4-{[(3R)-1-methylpiperidin-3-yl]amino}pyrido[3,4-d]pyridazin-1-yl)-5-(trifluoromethyl)benzamide formate;
5-[2-(difluoromethoxy)-4-(trifluoromethyl)phenyl]-N-[(3R)-1-methylpiperidin-3-yl]pyrido[2,3-d]pyridazin-8-amine formate;
8-[2-(difluoromethoxy)-4-(trifluoromethyl)phenyl]-N-[(3R)-1-methylpiperidin-3-yl]pyrido[2,3-d]pyridazin-5-amine formate;
2-{1-[(1-methylazepan-3-yl)amino]pyrido[3,4-d]pyridazin-4-yl}-5-(trifluoromethyl)phenol formate;
2-(4-{[(3R)-1-methylazepan-3-yl]amino}pyrido[3,4-d]pyridazin-1-yl)-5-(trifluoromethyl)phenol formate;
N-[(3R)-1-methylpiperidin-3-yl]-1-[2-(1-methyl-1H-pyrazol-4-yl)-4-(trifluoromethyl)phenyl]pyrido[3,4-d]pyridazin-4-amine formate;
[2-(4-{[(3R)-1-methylpiperidin-3-yl]amino}pyrido[3,4-d]pyridazin-1-yl)-5-(trifluoromethyl)phenyl]methanol formate;
N-[(3R)-1-methylpiperidin-3-yl]-1-(2,4,6-trimethylphenyl)pyrido[3,4-d]pyridazin-4-amine formate;
2-[(3R)-3-({1-[2-(difluoromethoxy)-4-(trifluoromethyl)phenyl]pyrido[3,4-d]pyridazin-4-yl}amino)piperidin-1-yl]ethan-1-ol formate;
6-chloro-3-fluoro-2-(4-{[(3R)-1-methylpiperidin-3-yl]amino}pyrido[3,4-d]pyridazin-1-yl)phenol formate;
6-chloro-3-fluoro-2-(5-{[(3R)-1-methylpiperidin-3-yl]amino}pyrido[2,3-d]pyridazin-8-yl)phenol formate;
N-[(3R)-1-methylpiperidin-3-yl]-1-[2-(2,2,2-trifluoroethyl)-4-(trifluoromethyl)phenyl]pyrido[3,4-d]pyridazin-4-amine formate;
3-methyl-4-(4-{[(3R)-1-methylpiperidin-3-yl]amino}pyrido[3,4-d]pyridazin-1-yl)benzonitrile formate;
1-(4-chloro-2,6-dimethylphenyl)-N-[(3R)-1-methylpiperidin-3-yl]pyrido[3,4-d]pyridazin-4-amine formate;
2,2,2-trifluoro-1-[3-hydroxy-4-(4-{[(3R)-piperidin-3-yl]amino}phthalazin-1-yl)phenyl]ethan-1-one formate;
5-(cyclopropylethynyl)-2-(4-{[(3R)-1-methylpiperidin-3-yl]amino}phthalazin-1-yl)phenol formate;
2-(4-{[(3R)-1-methylpiperidin-3-yl]amino}phthalazin-1-yl)-5-(prop-1-yn-1-yl)phenol formate;
N-[(3R)-1-methylpiperidin-3-yl]-1-[2-(prop-1-yn-1-yl)-4-(trifluoromethyl)phenyl]pyrido[3,4-d]pyridazin-4-amine formate;
2-{1-[(1,4-oxazepan-6-yl)amino]pyrido[3,4-d]pyridazin-4-yl}-5-(trifluoromethyl)phenol formate;
4-[2-(difluoromethoxy)-4-(trifluoromethyl)phenyl]-N-[(3R)-piperidin-3-yl]pyrido[3,4-d]pyridazin-1-amine formate;
4-[2-(difluoromethoxy)-4-(trifluoromethyl)phenyl]-N-[(3R)-1-ethylpiperidin-3-yl]pyrido[3,4-d]pyridazin-1-amine formate;
4-[2-(difluoromethoxy)-4-(trifluoromethyl)phenyl]-N-[(3R)-1-methylpiperidin-3-yl]-6,7-dihydro-5H-cyclopenta[d]pyridazin-1-amine formate;
5-(cyclopropyloxy)-2-(4-{[(3R)-1-methylpiperidin-3-yl]amino}pyrido[3,4-d]pyridazin-1-yl)phenol formate;
2-[(3R)-3-({4-[2-(difluoromethoxy)-4-(trifluoromethyl)phenyl]pyrido[3,4-d]pyridazin-1-yl}amino)piperidin-1-yl]ethan-1-ol formate;
4-[2-(difluoromethoxy)-4-methylphenyl]-N-[(3R)-1-methylpiperidin-3-yl]-6,7-dihydro-5H-cyclopenta[d]pyridazin-1-amine formate;
4-[2-(difluoromethoxy)phenyl]-N-[(3R)-1-methylpiperidin-3-yl]-6,7-dihydro-5H-cyclopenta[d]pyridazin-1-amine formate;
4-[4-cyclopropyl-2-(difluoromethoxy)phenyl]-N-[(3R)-1-methylpiperidin-3-yl]-6,7-dihydro-5H-cyclopenta[d]pyridazin-1-amine formate;
1-[4-cyclopropyl-2-(difluoromethoxy)phenyl]-N-[(3R)-1-methylpiperidin-3-yl]pyrido[3,4-d]pyridazin-4-amine formate;
1-[4-cyclopropyl-2-(difluoromethyl)phenyl]-N-[(3R)-1-methylpiperidin-3-yl]pyrido[3,4-d]pyridazin-4-amine formate;
N-[(3R)-azepan-3-yl]-1-[2-methoxy-4-(trifluoromethyl)phenyl]pyrido[3,4-d]pyridazin-4-amine formate;
2-{4-[(1,4-oxazepan-6-yl)amino]pyrido[3,4-d]pyridazin-1-yl}-5-(trifluoromethyl)phenol formate;
2-{4-[(azepan-4-yl)amino]pyrido[3,4-d]pyridazin-1-yl}-5-(trifluoromethyl)phenol formate;
2-(3-methyl-4-{[(3R)-1-methylpiperidin-3-yl]amino}[1,2]oxazolo[4,5-d]pyridazin-7-yl)-5-(trifluoromethyl)phenol formate;
2-(4-{[(3R)-1-(2-hydroxyethyl)piperidin-3-yl]amino}-3-methyl[1,2]oxazolo[4,5-d]pyridazin-7-yl)-5-(trifluoromethyl)phenol formate;
2-(4-{[(3R)-azepan-3-yl]amino}pyrido[3,4-d]pyridazin-1-yl)-5-(trifluoromethyl)phenol formate;
2-{4-[(1-methylazepan-4-yl)amino]pyrido[3,4-d]pyridazin-1-yl}-5-(trifluoromethyl)phenol formate;
2-{4-[(1,4-dimethyl-1,4-diazepan-6-yl)amino]pyrido[3,4-d]pyridazin-1-yl}-5-(trifluoromethyl)phenol formate;
ethyl (2S,5R)-5-({1-[2-hydroxy-4-(trifluoromethyl)phenyl]pyrido[3,4-d]pyridazin-4-yl}amino)piperidine-2-carboxylate hydrochloride;
5-methyl-2-(4-{[(3R)-1-methylpiperidin-3-yl]amino}pyrido[3,4-d]pyridazin-1-yl)benzene-1,3-diol formate;
2-(4-{[(1R,3S)-3-hydroxycyclohexyl]amino}pyrido[3,4-d]pyridazin-1-yl)-5-(trifluoromethyl)phenol formate;
2-(4-{[(1R,3R)-3-hydroxycyclohexyl]amino}pyrido[3,4-d]pyridazin-1-yl)-5-(trifluoromethyl)phenol formate;
3-fluoro-2-(4-{[(3R)-1-(2-hydroxyethyl)piperidin-3-yl]amino}pyrido[3,4-d]pyridazin-1-yl)-5-methylphenol formate;
2-(4-{[2-(dimethylamino)-2-methylpropyl]amino}pyrido[3,4-d]pyridazin-1-yl)-5-(trifluoromethyl)phenol formate;
N-{(3R)-1-[2-(difluoromethoxy)ethyl]piperidin-3-yl}-1-[2-methoxy-4-(trifluoromethyl)phenyl]pyrido[3,4-d]pyridazin-4-amine formate;
2-(4-{[(3 S,5S)-5-fluoro-1-methylpiperidin-3-yl]amino}pyrido[3,4-d]pyridazin-1-yl)-5-(trifluoromethyl)phenol formate;
2-(4-{[(3R,5R)-5-fluoro-1-methylpiperidin-3-yl]amino}pyrido[3,4-d]pyridazin-1-yl)-5-(trifluoromethyl)phenol formate;
5-chloro-3-fluoro-2-(4-{[(3R)-1-(2-hydroxyethyl)piperidin-3-yl]amino}pyrido[3,4-d]pyridazin-1-yl)phenol formate;
5-cyclopropyl-3-fluoro-2-(4-{[(3R)-1-(2-hydroxyethyl)piperidin-3-yl]amino}pyrido[3,4-d]pyridazin-1-yl)phenol formate;
3-fluoro-2-(4-{[(3R)-1-(2-hydroxyethyl)piperidin-3-yl]amino}pyrido[3,4-d]pyridazin-1-yl)phenol formate;
2-[4-({(3R)-1-[2-(difluoromethoxy)ethyl]piperidin-3-yl}amino)pyrido[3,4-d]pyridazin-1-yl]-5-(trifluoromethyl)phenol formate;
2-(4-{[(3R)-1-(propan-2-yl)piperidin-3-yl]amino}pyrido[3,4-d]pyridazin-1-yl)-5-(trifluoromethyl)phenol formate;
1-[2-(difluoromethyl)-4-methylphenyl]-N-[(3R)-1-methylpiperidin-3-yl]pyrido[3,4-d]pyridazin-4-amine formate;
2-(4-{[(1-methylpiperidin-4-yl)methyl]amino}pyrido[3,4-d]pyridazin-1-yl)-5-(trifluoromethyl)phenol formate;
2-[4-({[1-(2-hydroxyethyl)piperidin-4-yl]methyl}amino)pyrido[3,4-d]pyridazin-1-yl]-5-(trifluoromethyl)phenol formate;
2-(4-{[(3R,5R)-5-fluoropiperidin-3-yl]amino}pyrido[3,4-d]pyridazin-1-yl)-5-(trifluoromethyl)phenol formate;
5-ethyl-2-(4-{[(3R)-1-methylpiperidin-3-yl]amino}pyrido[3,4-d]pyridazin-1-yl)phenol formate;
N-{(3R)-1-[2-(difluoromethoxy)ethyl]piperidin-3-yl}-1-[2-(difluoromethoxy)-4-(trifluoromethyl)phenyl]pyrrolo[1,2-d][1,2,4]triazin-4-amine formate;
2-[(3R)-3-({1-[2-(difluoromethoxy)-4-(trifluoromethyl)phenyl]pyrrolo[1,2-d][1,2,4]triazin-4-yl}amino)piperidin-1-yl]ethan-1-ol formate;
2-(4-{[(3R)-1-methylpiperidin-3-yl]amino}imidazo[1,5-d][1,2,4]triazin-1-yl)-5-(trifluoromethyl)phenol formate;
1-[2-methoxy-4-(trifluoromethyl)phenyl]-N-[(3R)-1-methylpiperidin-3-yl]imidazo[1,5-d][1,2,4]triazin-4-amine formate;
2-(4-{[(3R)-1-ethylpiperidin-3-yl]amino}imidazo[1,5-d][1,2,4]triazin-1-yl)-5-(trifluoromethyl)phenol formate;
2-(4-{[(3R)-piperidin-3-yl]amino}pyrrolo[1,2-d][1,2,4]triazin-1-yl)-5-(trifluoromethoxy)phenol hydrochloride;
1-(4-chloro-2-methoxyphenyl)-N-[(3R)-1-methylpiperidin-3-yl]imidazo[1,5-d][1,2,4]triazin-4-amine formate;
5-chloro-2-(4-{[(3R)-1-methylpiperidin-3-yl]amino}imidazo[1,5-d][1,2,4]triazin-1-yl)phenol formate;
8-[2-methoxy-4-(trifluoromethyl)phenyl]-N-[(3R)-1-methylpiperidin-3-yl]imidazo[1,2-d][1,2,4]triazin-5-amine formate;
(3R,5R)-5-{[1-(4-cyclopropyl-2-fluoro-6-hydroxyphenyl)pyrrolo[1,2-d][1,2,4]triazin-4-yl]amino}-1-methylpiperidin-3-ol formate;
4-(4-bromo-2-methoxyphenyl)-N-[(3R)-piperidin-3-yl]pyrazolo[1,5-d][1,2,4]triazin-7-amine formate;
2-(4-{[(3R)-oxan-3-yl]amino}imidazo[1,5-d][1,2,4]triazin-1-yl)-5-(trifluoromethyl)phenol formate;
2-(5-{[(3R)-1-methylpiperidin-3-yl]amino}imidazo[1,2-d][1,2,4]triazin-8-yl)-5-(trifluoromethyl)phenol formate;
2-(4-{[(3R,5R)-5-fluoropiperidin-3-yl]amino}pyrrolo[1,2-d][1,2,4]triazin-1-yl)-5-(trifluoromethoxy)phenol hydrochloride;
4-[4-cyclopropyl-2-(trifluoromethoxy)phenyl]-2-methyl-N-[(3R)-1-methylpiperidin-3-yl]pyrazolo[1,5-d][1,2,4]triazin-7-amine formate;
2-methyl-N-[(3R)-1-methylpiperidin-3-yl]-4-[2-(trifluoromethoxy)phenyl]pyrazolo[1,5-d][1,2,4]triazin-7-amine formate;
2-(4-{[(2R)-2-hydroxypropyl]amino}pyrrolo[1,2-d][1,2,4]triazin-1-yl)-5-(trifluoromethoxy)phenol hydrochloride;
2-(8-fluoro-4-{[(3R)-1-methylpiperidin-3-yl]amino}pyrrolo[1,2-d][1,2,4]triazin-1-yl)-5-(trifluoromethoxy)phenol hydrochloride;
5-chloro-2-(4-{[(3R)-1-ethylpiperidin-3-yl]amino}imidazo[1,5-d][1,2,4]triazin-1-yl)phenol formate;
5-chloro-3-fluoro-2-(2-methyl-7-{[(3R)-1-methylpiperidin-3-yl]amino}pyrazolo[1,5-d][1,2,4]triazin-4-yl)phenol formate;
1-[2-(difluoromethoxy)-4-(trifluoromethyl)phenyl]-N-[(3S)-oxolan-3-yl]pyrrolo[1,2-d][1,2,4]triazin-4-amine formate;
2-(4-{[(3R)-1-methyl-1,2,3,6-tetrahydropyridin-3-yl]amino}imidazo[1,5-d][1,2,4]triazin-1-yl)-5-(trifluoromethyl)phenol formate;
2-(7-{[(3R)-1-methylpiperidin-3-yl]amino}[1,2,3]triazolo[1,5-d][1,2,4]triazin-4-yl)-5-(trifluoromethyl)phenol formate;
2-(4-{[(3S,4S)-4-fluoro-1-methylpiperidin-3-yl]amino}pyrrolo[1,2-d][1,2,4]triazin-1-yl)-5-(trifluoromethyl)phenol formate;
2-(4-{[(3R)-1-ethylpiperidin-3-yl]amino}pyrrolo[1,2-d][1,2,4]triazin-1-yl)-5-(trifluoromethyl)phenol formate;
2-(4-{[(3R)-1-(propan-2-yl)piperidin-3-yl]amino}pyrrolo[1,2-d][1,2,4]triazin-1-yl)-5-(trifluoromethyl)phenol formate;
1-[2-(difluoromethoxy)-6-fluoro-4-(trifluoromethyl)phenyl]-N-[(3R)-1-methylpiperidin-3-yl]pyrrolo[1,2-d][1,2,4]triazin-4-amine formate;
1-[4-chloro-2-(trifluoromethyl)phenyl]-N-[(3R)-1-methylpiperidin-3-yl]pyrrolo[1,2-d][1,2,4]triazin-4-amine formate;
5-cyclopropyl-2-(4-{[(3R)-1-(2,2-difluoroethyl)piperidin-3-yl]amino}pyrrolo[1,2-d][1,2,4]triazin-1-yl)-3-fluorophenol formate;
2-(4-{[(3R)-1-(2,2-difluoroethyl)piperidin-3-yl]amino}pyrrolo[1,2-d][1,2,4]triazin-1-yl)-3-fluoro-5-methylphenol formate;
2-(4-{[(3R,5R)-5-fluoro-1-methylpiperidin-3-yl]amino}imidazo[1,5-d][1,2,4]triazin-1-yl)-5-(trifluoromethyl)phenol formate;
2-(6-methyl-4-{[(3R)-1-methylpiperidin-3-yl]amino}imidazo[1,5-d][1,2,4]triazin-1-yl)-5-(trifluoromethyl)phenol formate;
5-bromo-2-(4-{[(3R)-1-(1-hydroxypropan-2-yl)piperidin-3-yl]amino}pyrrolo[1,2-d][1,2,4]triazin-1-yl)phenol formate;
5-cyclopropyl-2-(7-{[(3R)-1-ethylpiperidin-3-yl]amino}-2-methylpyrazolo[1,5-d][1,2,4]triazin-4-yl)-3-fluorophenol formate;
5-chloro-2-(4-{[(3R,5R)-5-fluoro-1-methylpiperidin-3-yl]amino}imidazo[1,5-d][1,2,4]triazin-1-yl)phenol formate;
5-ethyl-3-fluoro-2-(4-{[(3R)-1-methylpiperidin-3-yl]amino}pyrrolo[1,2-d][1,2,4]triazin-1-yl)phenol formate;
5-bromo-2-(4-{[(3R)-1-ethylpiperidin-3-yl]amino}pyrrolo[1,2-d][1,2,4]triazin-1-yl)phenol formate;
1-[2-(difluoromethoxy)-4-(trifluoromethyl)phenyl]-N-[(3R)-1-ethylpiperidin-3-yl]imidazo[1,5-d][1,2,4]triazin-4-amine formate;
2-[4-({[(2S)-1-ethylpyrrolidin-2-yl]methyl}amino)pyrrolo[1,2-d][1,2,4]triazin-1-yl]-3-fluoro-5-methylphenol formate;
5-cyclopropyl-2-[4-({[(2S)-1-ethylpyrrolidin-2-yl]methyl}amino)pyrrolo[1,2-d][1,2,4]triazin-1-yl]-3-fluorophenol formate;
2-(4-{[(3R)-1-ethylpiperidin-3-yl]amino}-8-fluoropyrrolo[1,2-d][1,2,4]triazin-1-yl)-5-(trifluoromethyl)phenol formate;
2-(8-fluoro-4-{[(3R,5R)-5-fluoro-1-methylpiperidin-3-yl]amino}pyrrolo[1,2-d][1,2,4]triazin-1-yl)-5-(trifluoromethyl)phenol formate;
(3R,5R)-5-({8-fluoro-1-[2-hydroxy-4-(trifluoromethyl)phenyl]pyrrolo[1,2-d][1,2,4]triazin-4-yl}amino)-1-methylpiperidin-3-ol formate;
2-(4-{[(3R,5R)-1-ethyl-5-fluoropiperidin-3-yl]amino}-8-fluoropyrrolo[1,2-d][1,2,4]triazin-1-yl)-5-(trifluoromethoxy)phenol formate;
5-bromo-2-(4-{[(3R)-1-(2-hydroxyethyl)piperidin-3-yl]amino}imidazo[1,5-d][1,2,4]triazin-1-yl)phenol formate;
1-[2-(difluoromethoxy)-4-(trifluoromethyl)phenyl]-N-[(3R)-1-methylpiperidin-3-yl]imidazo[1,5-d][1,2,4]triazin-4-amine formate; and
5-bromo-2-(4-{[(3R)-1-ethylpiperidin-3-yl]amino}imidazo[1,5-d][1,2,4]triazin-1-yl)phenol formate;
   wherein a form of the compound is selected from the group consisting of a hydrate, solvate, racemate, enantiomer, diastereomer, stereoisomer, and tautomer form thereof.

Another aspect of the invention provides a pharmaceutical composition comprising a therapeutically effective amount of a compound of Formulae I-XI or a pharmaceutically acceptable salt thereof and one or more pharmaceutically acceptable carriers.

Another aspect of the invention provides a method for treating or ameliorating a disease modulated by NLRP3 in a subject in need thereof comprising, administering to the subject an effective amount of the compound of Formulae I-XI.

Another aspect of the invention provides a method of treating or ameliorating a disease modulated by NLRP3 according to claim 15 selected from Alzheimer disease, Frontotemporal dementia (FTD), Huntington's disease, Parkinson's disease, Perioperative neurocognitive disorders, Post-cardiac arrest cognitive impairment, Poststroke cognitive impairment, Sepsis, Sepsis associated encephalopathy, Subarachnoid hemorrhage, Macular Degeneration, Retinal neovascularization, Uveitis, Colitis, Endothelial dysfunction, Gout, Pseudogout, Graft-versus-host-disease (GvHD), Systemic lupus erythematosus-lupus nephritis, Cryopyrin-associated periodic syndromes (CAPS), Cystic fibrosis, Sickle-cell disease, VCP-associated disease, Liver fibrosis, Nonalcoholic fatty liver disease (NASH), muscle atrophy, inherited and acquired myopathies, e.g. Duchenne Muscular Dystrophy (DMD), Hyperalgesia, Multiple sclerosis-associated neuropathic pain, Acute Kidney Injury, Chronic crystal nephropathy, Chronic Kidney Disease, asthma and allergic airway inflammation Diabetes-associated atherosclerosis, Diabetic encephalopathy, Diabetic kidney disease, Islet transplantation rejection, Obesity-associated renal disease, Oxalate-induced nephropathy, Renal fibrosis, Renal hypertension, Type I diabetes, Type II diabetes, Psoriasis, Hidradenitis suppurativa, Atherosclerosis and Cytokine Release Syndrome (CRS).

Another aspect of the invention provides a method of a compound of Formulae I-XI, wherein the effective amount of the compound is in a range of from about 0.001 mg/kg/day to about 500 mg/kg/day.

Another aspect of the invention provides a compound Formulae I-XI or a pharmaceutically acceptable salt thereof, for use in treating or ameliorating a disease modulated by NLRP3 selected from Alzheimer disease, Frontotemporal dementia (FTD), Huntington's disease, Parkinson's disease, Perioperative neurocognitive disorders, Post-cardiac arrest cognitive impairment, Poststroke cognitive impairment, Sepsis, Sepsis associated encephalopathy, Subarachnoid hemorrhage, Macular Degeneration, Retinal neovascularization, Uveitis, Colitis, Endothelial dysfunction, Gout, Pseudogout, Graft-versus-host-disease (GvHD), Systemic lupus erythematosus-lupus nephritis, Cryopyrin-associated periodic syndromes (CAPS), Cystic fibrosis, Sickle-cell disease, VCP-associated disease, Liver fibrosis, Nonalcoholic fatty liver disease (NASH), muscle atrophy, inherited and acquired myopathies, Hyperalgesia, Multiple sclerosis-associated neuropathic pain, Acute Kidney Injury, Chronic crystal nephropathy, Chronic Kidney Disease, asthma and allergic airway inflammation Diabetes-associated atherosclerosis, Diabetic encephalopathy, Diabetic kidney disease, Islet transplantation rejection, Obesity-associated renal disease, Oxalate-induced nephropathy, Renal fibrosis, Renal hypertension, Type I diabetes, Type II diabetes, Psoriasis, Hidradenitis suppurativa, Atherosclerosis and Cytokine Release Syndrome (CRS).

Another aspect of the invention provides a use of a compound of Formulae I-XI, wherein the effective amount of the compound is in a range of from about 0.001 mg/kg/day to about 500 mg/kg/day.

Another aspect of the invention provides a use of a compound Formulae I-XI in the preparation of a pharmaceutical composition for treating or ameliorating a disease modulated by NLRP3 in a subject in need thereof comprising, administering to the subject an effective amount of the compound or a form thereof in admixture with one or more of the pharmaceutically acceptable excipients.

Another aspect includes a compound of Formulae I-VIII, wherein R_{w} is selected from H, C₁₋₄alkyl, halogen, C₁₋₆alkoxy, halo-C₁₋₄alkyl, halo-C₁₋₄alkoxy, C₃₋₆cycloalkyl, amino or cyano.

Another aspect includes a compound of Formulae I-VIII, wherein R_{W} is hydrogen.

Another aspect includes a compound of Formulae I-VIII, wherein R_{w} is a C₁₋₆alkoxy or halo-C₁₋₄alkoxy.

Another aspect includes a compound of Formulae I-VIII, wherein R_{W} is OCHF₂ or OCF₃.

Another aspect includes a compound of Formulae I-VIII, wherein R_{W} is OCH₃.

Another aspect includes a compound of Formulae I-VIII, wherein R_{w} is a C₁₋₄alkyl or halo-C₁₋₄alkyl or C₃₋₆cycloalkyl.

Another aspect includes a compound of Formulae I-VIII, wherein R_{w} is CH₃.

Another aspect includes a compound of Formulae I-VIII, wherein R_{w} is c-Pr.

Another aspect includes a compound of Formulae I-VIII, wherein R_{w} is CF₃ or CHF₂.

Another aspect includes a compound of Formulae I-VIII, wherein R_{W} is a halogen selected from Br, Cl or F

Another aspect includes a compound of Formulae I-VIII, wherein R_{w} is F

Another aspect includes a compound of Formulae I-VIII, wherein R_{w} is C₁.

Another aspect includes a compound of Formulae I-VIII, wherein R_{w} is Br.

Another aspect includes a compound of Formulae I-VIII, wherein R_{w} is cyano.

Another aspect includes a compound of Formulae IX-XI, wherein R_{wa} is selected from hydrogen, hydroxyl, C₁₋₄alkyl, halogen, C₁₋₆alkoxy, halo-C₁₋₄alkyl, halo-C₁₋₄alkoxy, C₃. 6cycloalkyl, amino and cyano.

Another aspect includes a compound of Formulae IX-XI, wherein R_{wa} is hydrogen.

Another aspect includes a compound of Formulae IX-XI, wherein R_{wa} is OH.

Another aspect includes a compound of Formulae IX-XI, wherein R_{wa} is a C₁₋₆alkoxy or halo-C₁₋₄alkoxy.

Another aspect includes a compound of Formulae IX-XI, wherein R_{wa} is OCHF₂ or OCF₃.

Another aspect includes a compound of Formulae IX-XI, wherein R_{wa} is OCH₃.

Another aspect includes a compound of Formulae IX-XI, wherein R_{wa} is a C₁₋₄alkyl or halo-C₁₋₄alkyl or C₃₋₆cycloalkyl.

Another aspect includes a compound of Formulae IX-XI, wherein R_{wa} is CH₃.

Another aspect includes a compound of Formulae IX-XI, wherein R_{wa} is c-Pr.

Another aspect includes a compound of Formulae IX-XI, wherein R_{wa} is CF₃ or CHF₂.

Another aspect includes a compound of Formulae IX-XI, wherein R_{wa} is a halogen selected from Br, Cl or F

Another aspect includes a compound of Formulae IX-XI, wherein R_{wa} is F

Another aspect includes a compound of Formulae IX-XI, wherein R_{wa} is C₁.

Another aspect includes a compound of Formulae IX-XI, wherein R_{wa} is Br.

Another aspect includes a compound of Formulae IX-XI, wherein R_{wa} is cyano.

Another aspect includes a compound of Formulae I-III and V-IX, wherein W is selected from CH, CR′ and N.

Another aspect includes a compound of Formula I-III and V-X, wherein each R′ is independently heterocyclyl, heteroaryl, aryl, cycloalkyl, C₁₋₄alkyl, deutero-C₁₋₄alkyl, halo-C₁₋₄alkyl, C₁₋₄alkoxy, deutero-C₁₋₄alkoxy, or hydroxy-C₁₋₄alkyl; Another aspect includes a compound of Formula I-III and V-X, wherein each R′ is independently C₁₋₄alkyl, or halo-C₁₋₄alkyl.

Another aspect includes a compound of Formula I-III and V-X, wherein each R′ is independently methyl, ethyl, isopropyl, cyclopropyl, cyclobutyl, CF₃, or CHF₂.

Another aspect includes a compound of Formula I-III and V-X, wherein each R′ is independently C₁₋₄alkoxy, or deutero-C₁₋₄alkoxy.

Another aspect includes a compound of Formula I-III and V-X, wherein each R′ is independently OCH₃, OCD₃, OCHF₂, OCF₃, or OEt.

Another aspect includes a compound of Formula I-III and V-X, wherein each R′ is independently a halogen, selected from F, Cl, or Br.

Another aspect includes a compound of Formula I-III and V-X, wherein each R′ is independently F.

Another aspect includes a compound of Formula I-III and V-X, wherein each R′ is independently Cl.

Another aspect includes a compound of Formula I-III and V-X, wherein each R′ is independently Br.

Another aspect includes a compound of Formulae I-III, wherein Q is independently N or CH.

Another aspect includes a compound of Formulae I-III and IX, Q′, wherein Q′ is independently N, C or CH.

Another aspect of the invention provides that the core of the above Formulae I, and III-VIII may additionally be any of the following structures which may be optionally substituted with one or more R₄:

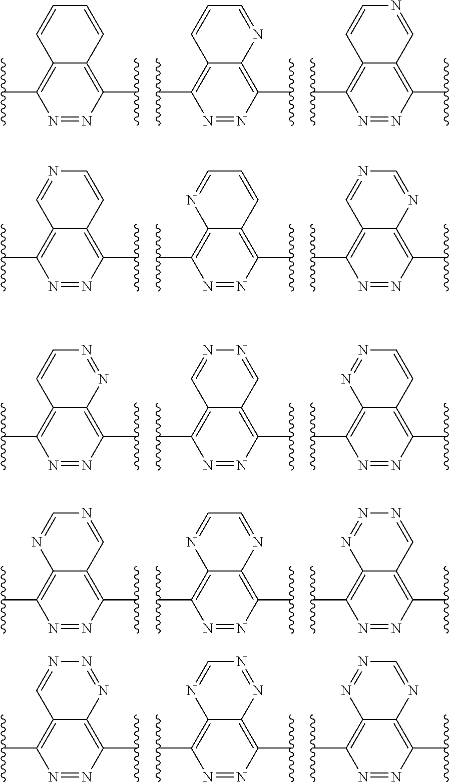

Another aspect of the invention provides that the core of the above Formulae I, IV or VIII may additionally be any of the following structures:

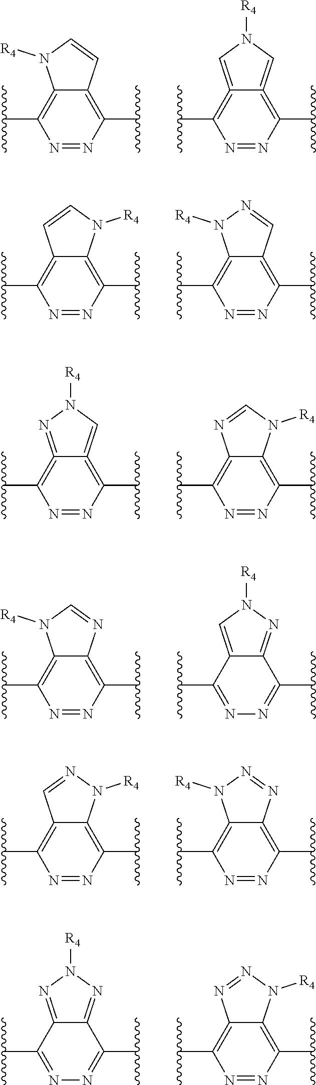

Another aspect of the invention provides that the core of Formulae IX-XI may additionally be, but are not limited to, any of the following structures which may be optionally substituted with one or more R₄:

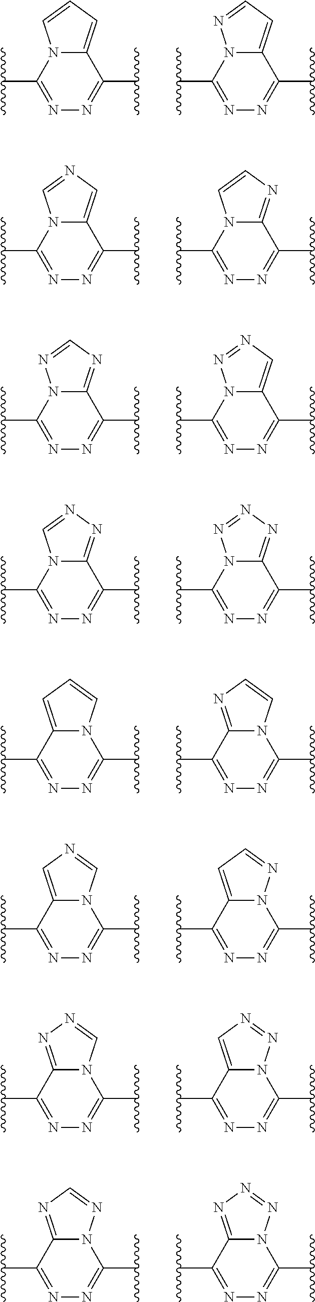

Another aspect includes a compound of Formula I-II, IV, and VIII-IX, wherein each A is independently absent, CH, CH₂, CRₐ, CHRₐ, CR₄, CHR₄, N, NH, NR₄ or NRₐ.

Another aspect includes a compound of Formula I-II, IV, and VIII-IX, wherein A′ is independently absent, CH, CH₂, CRₐ, CHRₐ, CHR₄, CHRₐ, N, NH, NR₄, NRₐ.

Another aspect includes a compound of Formula I-VII, wherein each Rₐ is independently a halogen, cyano, C₁₋₄alkyl, C₃₋₆cycloalkyl, haloC₁₋₄alkyl, C₁₋₄alkoxy, haloC₁₋₄alkoxy, amino, or C₁₋₄alkylamino.

Another aspect includes a compound of Formula I-VII, wherein each Rₐ is independently a halogen selected from F, Cl, or Br.

Another aspect includes a compound of Formula I-VII, wherein each Rₐ is independently a cyano.

Another aspect includes a compound of Formula I-VII, wherein each Rₐ is independently a C₁₋₄alkyl, which is selected from methyl, ethyl, n-propyl, isopropyl, n-butyl, isobutyl, sec-butyl, or tert-butyl.

Another aspect includes a compound of Formula I-VII, wherein that each Rₐ is independently a C₃₋₆cycloalkyl, which is selected from cylopropyl, cyclobutyl, cyclopentyl, or cyclohexyl.

Another aspect includes a compound of Formula I-VII, wherein each Rₐ is independently a cyclopropyl, or cyclobutyl.

Another aspect includes a compound of Formula I-VII, wherein each Rₐ is independently a C₁₋₄alkoxy or haloC₁₋₄alkoxy, which is selected from methoxy, ethoxy, isopropoxy, cyclopropoxy, difluoromethoxy, and trifluoromethoxy.

Another aspect includes a compound of Formula I-VII, wherein each Rₐ is independently an amino.

Another aspect includes a compound of Formula I-VII, wherein each Rₐ is independently a C₁₋₄alkylamino, which is selected from methylamino, ethylamino, N, N-dimethylamino, isopropylamino, or cyclopropylamino.

Another aspect includes a compound of Formulae I-XI wherein Y is NR_{1b}.

Another aspect includes that R_{1b} is hydrogen.

Another aspect includes that R_{1b} is a C₁₋₄alkyl, which is selected from methyl, ethyl, propyl, isopropyl, cyclopropyl, or butyl.

Another aspect includes that R_{1b} is methyl.

Another aspect includes a compound of Formulae I-VII and IX-XI, wherein Y is O.

Another aspect includes a compounds of Formulae I-VII and IX-XI, wherein Y is a carbon optionally substituted with R₁ₐ.

Another aspect includes a compound of Formula I-VII, wherein R₁ₐ is selected from hydrogen, or C₁₋₄alkyl.

Another aspect includes a compound of Formula I-VII, wherein R₁ₐ is hydrogen.

Another aspect includes a compound of Formula I-VII, wherein R₁ₐ is methyl.

Another aspect includes a compound of Formulae I-XI, wherein Z is C₃₋₆cycloalkyl selected from cyclopropyl, cylcobutyl, cyclopentyl or cyclohexyl, wherein each is optionally substituted with R₂

Another aspect includes a compound of Formulae I-XI, wherein Z is cyclopropyl, optionally substituted with R₂ selected from halogen, cyano, hydroxyl, C₁₋₄alkoxy, haloC₁₋₄alkoxy, C₁₋₄alkyl, haloC₁₋₄alkyl, or C₃₋₆cycloalkyl.

Another aspect includes a compound of Formulae I-XI, wherein Z is cyclopropyl, optionally substituted with R₂ selected from F, CN, OH, MeO, EtO, iPrO, cPrO, CHF₂O, CF₃O, Me, Et, CHF₂, CF₃, cPr, or cBu.

Another aspect includes a compound of Formulae I-XI, wherein Z is cyclobutyl, optionally substituted with R₂ selected from halogen, cyano, hydroxyl, C₁₋₄alkoxy, haloC₁₋₄alkoxy, C₁₋₄alkyl, haloC₁₋₄alkyl, or C₃₋₆cycloalkyl.

Another aspect includes a compound of Formulae I-XI, wherein Z is cyclobutyl, optionally substituted with R₂ selected from F, CN, OH, MeO, EtO, iPrO, cPrO, CHF₂O, CF₃O, Me, Et, CHF₂, CF₃, cPr, or cBu.

Another aspect includes a compound of Formulae I-XI, wherein Z is cyclopentyl, optionally substituted with R₂ selected from halogen, cyano, hydroxyl, C₁₋₄alkoxy, haloC₁₋₄alkoxy, C₁₋₄alkyl, haloC₁₋₄alkyl, or C₃₋₆cycloalkyl.

Another aspect includes a compound of Formulae I-XI, wherein Z is cyclopentyl, optionally substituted with R₂ selected from F, CN, OH, MeO, EtO, iPrO, cPrO, CHF₂O, CF₃O, Me, Et, CHF₂, CF₃, cPr, or cBu.

Another aspect includes a compound of Formulae I-XI, wherein Z is cyclohexyl, optionally substituted with R₂ selected from halogen, cyano, hydroxyl, C₁₋₄alkoxy, haloC₁₋₄alkoxy, C₁₋₄alkyl, haloC₁₋₄alkyl, or C₃₋₆cycloalkyl.

Another aspect includes a compound of Formulae I-XI, wherein Z is cyclohexyl, optionally substituted with R₂ selected from F, CN, OH, MeO, EtO, iPrO, cPrO, CHF₂O, CF₃O, Me, Et, CHF₂, CF₃, cPr, or cBu.

Another aspect includes a compound of Formulae I-XI, wherein Z is heterocyclyl, wherein each is optionally substituted with R₂ selected from halogen, cyano, hydroxyl, C₁₋₄alkoxy, haloC₁₋₄alkoxy, C₁₋₄alkyl, haloC₁₋₄alkyl, or C₃₋₆cycloalkyl.

Another aspect includes a compound of Formulae I-XI, wherein Z is piperidinyl, tetrahydro-2H-pyran, tetrahydrofuran, or pyrrolidinyl, wherein each is optionally substituted with R₂ selected from halogen, cyano, hydroxyl, C₁₋₄alkoxy, haloC₁₋₄alkoxy, C₁₋₄alkyl, haloC₁₋₄alkyl, or C₃₋₆cycloalkyl.

Another aspect includes a compound of Formulae I-XI, wherein Z is piperidinyl, optionally substituted with R₂ selected from F, CN, OH, MeO, EtO, iPrO, cPrO, CHF₂O, CF₃O, Me, Et, iPr, CHF₂, CF₃, cPr, cBu, —CH₂CH₂OH, —CH₂CH₂OCHF₂, —CH₂CH₂OCF₃, tetrahydrofuranyl, or tetrahydropyranyl.

Another aspect includes a compound of Formulae I-XI, wherein Z is pyrrolidinyl, optionally substituted with R₂ selected from F, CN, OH, MeO, EtO, iPrO, cPrO, CHF₂O, CF₃O, Me, Et, iPr, CHF₂, CF₃, cPr, cBu, —CH₂CH₂OH, —CH₂CH₂OCHF₂, —CH₂CH₂OCF₃, tetrahydrofuranyl, or tetrahydropyranyl.

Another aspect includes a compound of Formulae I-XI, wherein Z is tetrahydropyranyl, optionally substituted with R₂ selected from F, CN, OH, MeO, EtO, iPrO, cPrO, CHF₂O, CF₃O, Me, Et, iPr, CHF₂, CF₃, cPr, cBu, —CH₂CH₂OH, —CH₂CH₂OCHF₂, —CH₂CH₂OCF₃, tetrahydrofuranyl, or tetrahydropyranyl.

Another aspect includes a compound of Formulae I-XI, wherein Z is tetrahydrofuranyl, optionally substituted with R₂ selected from F, CN, OH, MeO, EtO, iPrO, cPrO, CHF₂O, CF₃O, Me, Et, iPr, CHF₂, CF₃, cPr, cBu, —CH₂CH₂OH, —CH₂CH₂OCHF₂, —CH₂CH₂OCF₃, tetrahydrofuranyl, or tetrahydropyranyl.

Another aspect includes a compound of Formulae I-XI, wherein Z is C₁₋₄alkyl, optionally substituted with R₂ selected from halogen, cyano, hydroxyl, C₁₋₄alkoxy, haloC₁₋₄alkoxy, C₁₋₄alkyl, haloC₁₋₄alkyl, or C₃₋₆cycloalkyl.

Another aspect includes a compound of Formulae I-XI, wherein Z is C₁₋₄alkyl, optionally substituted with R₂ selected from F, CN, OH, MeO, EtO, iPrO, cPrO, CHF₂O, CF₃O, Me, Et, CHF₂, CF₃, cPr, or cBu.

An aspect of the present description includes a method for preventing, treating or ameliorating any disease that is mediated by NLRP3 in a subject in need thereof comprising, administering to the subject an effective amount of a compound of Formulae I-XI or a form thereof.

Another aspect includes a compound of Formulae I-XI, wherein R₂ is halogen selected from bromo, chloro, fluoro, and iodo.

Another aspect includes a compound of Formulae I-XI, wherein R₂ is fluoro.

Another aspect includes a compound of Formulae I-XI, wherein R₂ is hydroxyl.

Another aspect includes a compound of Formulae I-XI, wherein R₂ is C₁₋₄alkyl selected from methyl, ethyl, propyl, isopropyl, butyl, isobutyl, sec-butyl, and tert-butyl.

Another aspect includes a compound of Formulae I-XI, wherein R₂ is C₁₋₄alkyl selected from methyl and ethyl.

Another aspect includes a compound of Formulae I-XI, wherein R₂ is methyl.

Another aspect includes a compound of Formulae I-XI, wherein R₂ is ethyl.

Another aspect includes a compound of Formulae I-XI, wherein R₂ is amino Another aspect includes a compound of Formulae I-XI, wherein R₂ is C₁₋₆alkylamino, wherein C₁₋₄alkyl is selected from methyl, ethyl, propyl, isopropyl, butyl, isobutyl, sec-butyl, tert-butyl, 2-methylbutyl, and 3-methylpentyl.

Another aspect includes a compound of Formulae I-XI, wherein R₂ is C₁₋₆alkylamino, wherein C₁₋₄alkyl is selected from methyl, ethyl, isopropyl, tert-butyl, 2methylbutyl, and 3-methylpentyl.

Another aspect includes a compound of Formulae I-XI, wherein R₂ is methylamino.

Another aspect includes a compound of Formulae I-XI, wherein R₂ is ethylamino.

Another aspect includes a compound of Formulae I-XI, wherein R₂ is isopropylamino.

Another aspect includes a compound of Formulae I-XI, wherein R₂ is tert-butylamino.

Another aspect includes a compound of Formulae I-XI, wherein R₂ is 2-methylbutyl-2-amino.

Another aspect includes a compound of Formulae I-XI, wherein R₂ is 3-methylpentyl-3-amino.

Another aspect includes a compound of Formulae I-XI, wherein R₂ is (C₁₋₆alkyl)₂amino, wherein C₁₋₄alkyl is each independently selected from selected from methyl, ethyl, isopropyl, tert-butyl, 2-methylbutyl, and 3-methylpentyl.

Another aspect includes a compound of Formulae I-XI, wherein R₂ is (C₁₋₆alkyl)₂amino, wherein C₁₋₄alkyl is methyl or ethyl.

Another aspect includes a compound of Formulae I-XI, wherein R₂ is dimethylamino or diethylamino.

Another aspect includes a compound of Formulae I-XI, wherein R₂ is dimethylamino.

Another aspect includes a compound of Formulae I-XI, wherein R₂ is diethylamino.

Another aspect includes a compound of Formulae I-XI, wherein R₂ is halo-C₁₋₄ alkylamino, wherein C₁₋₄alkyl is selected from methyl, ethyl, propyl, isopropyl, butyl, isobutyl, sec-butyl, and tert-butyl, partially or completely substituted with one or more halogen atoms where allowed by available valences.

Another aspect includes a compound of Formulae I-XI, wherein R₂ is halo-C₁₋₄ alkylamino, wherein C₁₋₄alkyl is selected from isopropyl and tert-butyl, partially or completely substituted with one or more halogen atoms where allowed by available valences.

Another aspect includes a compound of Formulae I-XI, wherein R₂ is 1-fluoro-2-methylpropan-2-amino or 1-fluoropropan-2-amino.

Another aspect includes a compound of Formulae I-XI, wherein R₂ is hydroxy-C₁₋₄alkylamino, wherein C₁₋₄alkyl is selected from methyl, ethyl, propyl, isopropyl, butyl, isobutyl, sec-butyl, and tert-butyl, partially or completely substituted with one or more hydroxy groups where allowed by available valences.

Another aspect includes a compound of Formulae I-XI, wherein R₂ is hydroxy-C₁₋₄alkylamino, wherein C₁₋₄alkyl is selected from ethyl and propyl, partially or completely substituted with one or more hydroxy groups where allowed by available valences.

Another aspect includes a compound of Formulae I-XI, wherein R₂ is 2hydroxyethylamino or 3-hydroxypropylamino.

Another aspect includes a compound of Formulae I-XI, wherein R₂ is C₁₋₄alkoxy-C₁₋₄alkyl-amino, wherein C₁₋₄alkoxy is selected from methoxy, ethoxy, propoxy, isopropoxy, butoxy and tert-butoxy, and C₁₋₄alkyl is selected from methyl, ethyl, propyl, isopropyl, butyl, isobutyl, sec-butyl, and tert-butyl.

Another aspect includes a compound of Formulae I-XI, wherein R₂ is C₁₋₄alkoxy-C₁₋₄alkyl-amino, wherein C₁₋₄alkoxy is methoxy and C₁₋₄alkyl is selected propyl, isopropyl, and tert-butyl.

Another aspect includes a compound of Formulae I-XI, wherein R₂ is 1methoxypropan-2-amino or 1-methoxy-2-methylpropan-2-amino.

Another aspect includes a compound of Formulae I-XI, wherein R₂ is C₁₋₄alkylaminoC₁₋₄alkyl, wherein each C₁₋₄alkyl is independently selected from methyl, ethyl, propyl, isopropyl, butyl, isobutyl, sec-butyl, and tert-butyl.

Another aspect includes a compound of Formulae I-XI, wherein R₂ is C₁₋₄alkylaminoC₁₋₄alkyl, wherein each C₁₋₄alkyl is independently selected from methyl, ethyl, isopropyl, and tert-butyl.

Another aspect includes a compound of Formulae I-XI, wherein R₂ is methylaminomethyl, propan-2-yl-aminomethyl, propan-2-yl-aminoethyl, or tert-butylaminomethyl.

Another aspect includes a compound of Formulae I-XI, wherein R₂ is (C₁₋₄alkylamino)₂C₁₋₄alkyl, wherein each C₁₋₄alkyl is independently selected from methyl, ethyl, propyl, isopropyl, butyl, isobutyl, sec-butyl, and tert-butyl.

Another aspect includes a compound of Formulae I-XI, wherein R₂ is (C₁₋₄alkylamino)₂C₁₋₄alkyl, wherein each C₁₋₄alkyl is methyl.

Another aspect includes a compound of Formulae I-XI, wherein R₂ is dimethylaminomethyl.

Another aspect includes a compound of Formulae I-XI, wherein R₂ is C₁₋₄alkoxy selected from methoxy, ethoxy, propoxy, isopropoxy, butoxy and tert-butoxy.

Another aspect includes a compound of Formulae I-XI, wherein R₂ methoxy.

Another aspect includes a compound of Formulae I-XI, wherein R₂ is C₃₋₁₀cycloalkyl-amino, wherein C₃₋₁₀cycloalkyl is selected from cyclopropyl, cylcobutyl, cyclopentyl, cyclohexyl, cycloheptyl, cyclooctyl, bicyclo[2.2.1]hexanyl, and adamantyl.

Another aspect includes a compound of Formulae I-XI, wherein R₂ is C₃₋₁₀cycloalkyl-amino, wherein C₃₋₁₀cycloalkyl is selected from cyclopropyl, cylcobutyl, cyclopentyl, bicyclo[2.2.1]hexanyl, and adamantyl.

Another aspect includes a compound of Formulae I-XI, wherein R₂ is C₃₋₁₀cycloalkyl-amino-C₁₋₄alkyl, wherein C₃₋₁₀cycloalkyl is selected from cyclopropyl, cylcobutyl, cyclopentyl, cyclohexyl, cycloheptyl, cyclooctyl, bicyclo[2.2.1]hexanyl, and adamantyl and C₁₋₄alkyl is selected from methyl, ethyl, propyl, and butyl.

Another aspect includes a compound of Formulae I-XI, wherein R₂ is C₃₋₁₀cycloalkyl-amino-C₁₋₄alkyl, wherein C₃₋₁₀cycloalkyl is selected from cyclopropyl, cylcobutyl, and cyclopentyl, and C₁₋₄alkyl is methyl.

Another aspect includes a compound of Formulae I-XI, wherein R₂ is cyclopropylaminomethyl, cyclobutylaminomethyl, or cyclopentylaminomethyl.

Another aspect includes a compound of Formulae I-XI, wherein R₂ is heteroaryl-C₁₋₄alkyl-amino, wherein heteroaryl is selected from thienyl, 1Hpyrazolyl, 1Himidazolyl, 1,3thiazolyl, oxazolyl, 1,2,4oxadiazolyl, 1,3,4oxadiazolyl, 1,2,4thiadiazolyl, 1H-tetrazolyl, 2H-tetrazolyl, pyridinyl, pyrimidinyl, pyridazinyl, 1,2,4triazinyl, 1,3,5-triazinyl, 1Hindolyl, 1Hindazolyl, 2Hindazolyl, indolizinyl, benzofuranyl, benzothienyl, 1Hbenzimidazolyl, 1,3benzoxazolyl, 1,3benzothiazolyl, 1,3-benzodioxolyl, 1,2,3-benzotriazolyl, 9Hpurinyl, quinolinyl, isoquinolinyl, and quinoxalinyl, and C₁₋₄alkyl is selected from methyl, ethyl, propyl, and butyl.

Another aspect includes a compound of Formulae I-XI, wherein R₂ is heteroaryl-C₁₋₄alkyl-amino, wherein heteroaryl is pyridinyl, and C₁₋₄alkyl is methyl.

Another aspect includes a compound of Formulae I-XI, wherein R₂ is pyridin-2-yl-methylamino.

Another aspect includes a compound of Formulae I-XI, wherein R₂ is heterocyclyl-amino, wherein heterocyclyl is selected from azetidinyl, oxetanyl, pyrrolidinyl, tetrahydrofuranyl, piperidinyl, tetrahydropyranyl, 8-azabicyclo[3.2.1]octanyl, and 8oxabicyclo[3.2.1]octanyl.

Another aspect includes a compound of Formulae I-XI, wherein R₂ is heterocyclyl-amino, wherein heterocyclyl is selected from oxetanyl and tetrahydropyranyl.

Another aspect includes a compound of Formulae I-XI, wherein R₂ is oxetanylamino or tetrahyropyranylamino.

Another aspect includes a compound of Formulae I-XI, wherein R₂ is heterocyclyl-amino-C₁₋₄alkyl, wherein heterocyclyl is selected from azetidinyl, oxetanyl, pyrrolidinyl, tetrahydrofuranyl, piperidinyl, oxanyl, 8-azabicyclo[3.2.1]octanyl, and 8oxabicyclo[3.2.1]octanyl, and C₁₋₄alkyl is selected from methyl, ethyl, propyl, and butyl.

Another aspect includes a compound of Formulae I-XI, wherein R₂ is heterocyclyl-amino-C₁₋₄alkyl, wherein heterocyclyl is selected from tetrahydrofuranyl, oxanyl, and 8-oxabicyclo[3.2.1]octanyl, and C₁₋₄alkyl is methyl.

Another aspect includes a compound of Formulae I-XI, wherein R₂ is oxanylaminomethyl, tetrahydrofuranylaminomethyl, and 8-oxabicyclo[3.2.1]octanylamino.

Another aspect includes a compound of Formulae I-XI, wherein R₂ is heterocyclyl-amino-C₃₋₁₀cycloalkyl, wherein heterocyclyl is selected from azetidinyl, oxetanyl, pyrrolidinyl, tetrahydrofuranyl, piperidinyl, oxanyl, 8-azabicyclo[3.2.1]octanyl, and 8oxabicyclo[3.2.1]octanyl, and C₃₋₁₀cycloalkyl is selected from cyclopropyl, cylcobutyl, cyclopentyl, and cyclohexyl.

Another aspect includes a compound of Formulae I-XI, wherein R₂ is heterocyclyl-amino-C₃₋₁₀ cycloalkyl, wherein heterocyclyl is oxanyl, and C₃₋₁₀cycloalkyl is cyclopropyl.

Another aspect includes a compound of Formulae I-XI, wherein R₂ is oxanylaminocyclopropyl.

One aspect includes a compound of Formulae I-XI, wherein R₃ is halogen, hydroxyl, cyano, C₁₋₄alkyl, deutero-C₁₋₄alkyl, halo-C₁₋₄alkyl, amino, C₁₋₄alkoxy, and haloC₁₋₄alkoxy.

Another aspect includes a compound of Formulae I-XI, wherein R₃ is halogen and C₁₋₄alkyl.

Another aspect includes a compound of Formulae I-XI, wherein R₃ is halogen selected from bromo, chloro, fluoro, and iodo.

Another aspect includes a compound of Formulae I-XI, wherein R₃ is fluoro.

Another aspect includes a compound of Formulae I-XI, wherein R₃ is C₁₋₄alkyl selected from methyl, ethyl, propyl, isopropyl, butyl, isobutyl, sec-butyl, and tert-butyl.

Another aspect includes a compound of Formulae I-XI, wherein R₃ is methyl.

One aspect includes a compound of Formulae I-XI, wherein R₄ is selected from halogen, hydroxyl, cyano, C₁₋₄alkyl, deutero-C₁₋₄alkyl, halo-C₁₋₄alkyl, amino, C₁₋₄alkylamino, (C₁₋₄alkyl)₂amino, C₁₋₄alkoxy, halo-C₁₋₄alkoxy, heteroaryl, heterocyclyl, and phenyl, wherein heteroaryl is a 5-6 membered monocyclic or 6-10 membered bicyclic ring system having 1, 2, 3, or 4 heteroatom ring members independently selected from N, O, and S, wherein heterocyclyl is a saturated or partially unsaturated 3-6 membered monocyclic or 9-10 membered bicyclic ring system having 1, 2, or 3 heteroatom ring members selected from N, O, and S, and wherein each instance of phenyl, heteroaryl or heterocyclyl is optionally substituted with 1 or 2 substituents each selected from R₅.

Another aspect includes a compound of Formulae I-XI, wherein R₄ is selected from halogen, C₁₋₄alkoxy, heteroaryl, and phenyl.

Another aspect includes a compound of Formulae I-XI, wherein R₄ is halogen selected from bromo, chloro, fluoro, and iodo.

Another aspect includes a compound of Formulae I-XI, wherein R₄ is fluoro.

Another aspect includes a compound of Formulae I-XI, wherein R₄ is C₁₋₄alkoxy selected from methoxy, ethoxy, propoxy, isopropoxy, butoxy, and tert-butoxy.

Another aspect includes a compound of Formulae I-XI, wherein R₄ methoxy.

Another aspect includes a compound of Formulae I-XI, wherein R₄ is heteroaryl, wherein heteroaryl is a 5-6 membered monocyclic or 6-10 membered bicyclic ring system having 1, 2, 3, or 4 heteroatom ring members independently selected from N, O, and S, optionally substituted with 1 or 2 substituents each selected from R₅.

Another aspect includes a compound of Formulae I-XI, wherein R₄ is phenyl, 1 or 2 substituents each selected from R₅.

One aspect includes a compound of Formulae I-XI, wherein R₅ is selected from halogen, hydroxyl, cyano, nitro, C₁₋₄alkyl, deutero-C₁₋₄alkyl, halo-C₁₋₄alkyl, amino, C₁₋₄alkylamino, (C₁₋₄alkyl)₂amino, aminoC₁₋₄alkyl, hydroxylC₁₋₄alkyl, C₁₋₄alkylcarbonyl, C₁₋₄alkoxy, C₁₋₄alkylthio, halo-C₁₋₄alkoxy, and C₃₋₁₀cycloalkyl.

Another aspect includes a compound of Formulae I-XI, wherein R₅ is selected from halogen, hydroxyl, cyano, nitro, C₁₋₄alkyl, deutero-C₁₋₄alkyl, amino, C₁₋₄alkylamino, aminoC₁₋₄alkyl, hydroxylC₁₋₄alkyl, C₁₋₄alkylcarbonyl, C₁₋₄alkoxy, C₁₋₄alkylthio, and C₃₋₁₀cycloalkyl.

Another aspect includes a compound of Formulae I-XI, wherein R₅ is halogen selected from bromo, chloro, fluoro, and iodo.

Another aspect includes a compound of Formulae I-XI, wherein R₅ is halogen selected from bromo, chloro and fluoro.

Another aspect includes a compound of Formulae I-XI, wherein R₅ is chloro.

Another aspect includes a compound of Formulae I-XI, wherein R₅ is fluoro.

Another aspect includes a compound of Formulae I-XI, wherein R₅ is hydroxy.

Another aspect includes a compound of Formulae I-XI, wherein R₅ is cyano.

Another aspect includes a compound of Formulae I-XI, wherein R₅ is nitro.

Another aspect includes a compound of Formulae I-XI, wherein R₅ is C₁₋₄alkyl selected from methyl, ethyl, propyl, isopropyl, butyl, isobutyl, sec-butyl, and tert-butyl.

Another aspect includes a compound of Formulae I-XI, wherein R₅ is methyl.

Another aspect includes a compound of Formulae I-XI, wherein R₅ is deutero-C₁₋₄alkyl wherein C₁₋₄alkyl is selected from methyl, ethyl, propyl, isopropyl, butyl, isobutyl, sec-butyl, and tert-butyl partially or completely substituted with one or more deuterium atoms where allowed by available valences.

Another aspect includes a compound of Formulae I-XI, wherein R₅ is (²H₃)methyl.

Another aspect includes a compound of Formulae I-XI, wherein R₅ is amino.

Another aspect includes a compound of Formulae I-XI, wherein R₅ is C₁₋₆alkylamino wherein C₁₋₄alkyl is selected from methyl, ethyl, propyl, isopropyl, butyl, isobutyl, sec-butyl, tert-butyl, 2-methylbutyl, and 3-methylpentyl.

Another aspect includes a compound of Formulae I-XI, wherein R₅ is C₁₋₄alkylamino wherein C₁₋₄alkyl is methyl.

Another aspect includes a compound of Formulae I-XI, wherein R₅ is methylamino.

Another aspect includes a compound of Formulae I-XI, wherein R₅ is aminoC₁₋₄alkyl wherein C₁₋₄alkyl is selected from methyl, ethyl, propyl, isopropyl, butyl, isobutyl, sec-butyl, and tert-butyl.

Another aspect includes a compound of Formulae I-XI, wherein R₅ is aminoC₁₋₄alkyl wherein C₁₋₄alkyl is methyl.

Another aspect includes a compound of Formulae I-XI, wherein R₅ is aminomethyl.

Another aspect includes a compound of Formulae I-XI, wherein R₅ is hydroxylC₁₋₄alkyl, wherein C₁₋₄alkyl is selected from methyl, ethyl, propyl, isopropyl, butyl, isobutyl, sec-butyl, and tert-butyl partially or completely substituted with one or more hydroxyl groups where allowed by available valences.

Another aspect includes a compound of Formulae I-XI, wherein R₅ is hydroxylC₁₋₄alkyl, wherein C₁₋₄alkyl is methyl substituted with one hydroxyl group.

Another aspect includes a compound of Formulae I-XI, wherein R₅ is hydroxymethyl.

Another aspect includes a compound of Formulae I-XI, wherein R₅ is C₁₋₄alkylcarbonyl, wherein C₁₋₄alkyl is selected from methyl, ethyl, propyl, isopropyl, butyl, isobutyl, sec-butyl, and tert-butyl.

Another aspect includes a compound of Formulae I-XI, wherein R₅ is C₁₋₄alkylcarbonyl, wherein C₁₋₄alkyl is methyl.

Another aspect includes a compound of Formulae I-XI, wherein R₅ is CH₃C(O)—.

Another aspect includes a compound of Formulae I-XI, wherein R₅ is C₁₋₄alkoxy selected from methoxy, ethoxy, propoxy, isopropoxy, butoxy, and tert-butoxy.

Another aspect includes a compound of Formulae I-XI, wherein R₅ methoxy.

Another aspect includes a compound of Formulae I-XI, wherein R₅ is C₁₋₄alkylthio selected from methylthio, ethylthio, propylthio, isopropylthio, butylthio, and tert-butylthio.

Another aspect includes a compound of Formulae I-XI, wherein R₅ methylthio.

Another aspect includes a compound of Formulae I-XI, wherein R₅ is C₃₋₁₀cycloalkyl selected from cyclopropyl, cylcobutyl, cyclopentyl, cyclohexyl, cycloheptyl, cyclooctyl, bicyclo[2.2.1]hexanyl, and adamantly.

Another aspect of the invention provides a method of treating or ameliorating a disease modulated by NLRP3 with a compound of Formulae I-XI wherein said disease is selected from Alzheimer disease, Frontotemporal dementia (FTD), Huntington's disease, Parkinson's disease, Perioperative neurocognitive disorders, Post-cardiac arrest cognitive impairment, Poststroke cognitive impairment, Sepsis, Sepsis associated encephalopathy, Subarachnoid hemorrhage, Macular Degeneration, Retinal neovascularization, Uveitis, Colitis, Endothelial dysfunction, Gout, Pseudogout, Graft-versus-host-disease (GvHD), Systemic lupus erythematosus-lupus nephritis, Cryopyrin-associated periodic syndromes (CAPS), Cystic fibrosis, Sickle-cell disease, VCP-associated disease, Liver fibrosis, Nonalcoholic fatty liver disease (NASH), muscle atrophy, inherited and acquired myopathies, e.g. Duchenne Muscular Dystrophy (DMD), Hyperalgesia, Multiple sclerosis-associated neuropathic pain, Acute Kidney Injury, Chronic crystal nephropathy, Chronic Kidney Disease, asthma and allergic airway inflammation Diabetes-associated atherosclerosis, Diabetic encephalopathy, Diabetic kidney disease, Islet transplantation rejection, Obesity-associated renal disease, Oxalate-induced nephropathy, Renal fibrosis, Renal hypertension, Type I diabetes, Type II diabetes, Psoriasis, Hidradenitis suppurativa, Atherosclerosis and Cytokine Release Syndrome (CRS).

Another aspect of the invention provides a method of treating a subject with a compound of Formulae I-XI, wherein the effective amount of the compound is in a range of from about 0.001 mg/kg/day to about 500 mg/kg/day.

Another aspect of the invention provides a compound of Formulae I-XI or a pharmaceutically acceptable salt thereof, for use in treating or ameliorating a disease modulated by NLRP3 selected from Alzheimer disease, Frontotemporal dementia (FTD), Huntington's disease, Parkinson's disease, Perioperative neurocognitive disorders, Post-cardiac arrest cognitive impairment, Poststroke cognitive impairment, Sepsis, Sepsis associated encephalopathy, Subarachnoid hemorrhage, Macular Degeneration, Retinal neovascularization, Uveitis, Colitis, Endothelial dysfunction, Gout, Pseudogout, Graft-versus-host-disease (GvHD), Systemic lupus erythematosus-lupus nephritis, Cryopyrin-associated periodic syndromes (CAPS), Cystic fibrosis, Sickle-cell disease, VCP-associated disease, Liver fibrosis, Nonalcoholic fatty liver disease (NASH), muscle atrophy, inherited and acquired myopathies, Hyperalgesia, Multiple sclerosis-associated neuropathic pain, Acute Kidney Injury, Chronic crystal nephropathy, Chronic Kidney Disease, asthma and allergic airway inflammation Diabetes-associated atherosclerosis, Diabetic encephalopathy, Diabetic kidney disease, Islet transplantation rejection, Obesity-associated renal disease, Oxalate-induced nephropathy, Renal fibrosis, Renal hypertension, Type I diabetes, Type II diabetes, Psoriasis, Hidradenitis suppurativa, Atherosclerosis and Cytokine Release Syndrome (CRS).

Another aspect of the invention provides a use of a compound of Formulae I-XI, wherein the effective amount of the compound is in a range of from about 0.001 mg/kg/day to about 500 mg/kg/day.

Another aspect of the invention provides a use of a compound of Formulae I-XI in the preparation of a pharmaceutical composition for treating or ameliorating a disease modulated by NLRP3 in a subject in need thereof comprising, administering to the subject an effective amount of the compound or a form thereof in admixture with one or more of the pharmaceutically acceptable excipients.

The application further provides a compound, composition, use or method as described herein.

### METHOD OF USE OF THE INVENTION

There is evidence for a role of NLRP3-induced IL-I and IL-18 in the inflammatory responses occurring in connection with, or as a result of, a multitude of different disorders (Menu et al, Clinical and Experimental Immunology, 2011, 166, 1-15; Strowig et al, Nature, 2012, 481, 278-286). NLRP3 mutations have been found to be responsible for a set of rare autoinflammatory diseases known as CAPS (Ozaki et al, J. Inflammation Research, 2015, 8, 15-27; Schroder et al, Cell, 2010, 140:821-832; Menu et al, Clinical and Experimental Immunology, 2011, 166, 1-15). CAPS are heritable diseases characterized by recurrent fever and inflammation and are comprised of three autoinflammatory disorders that form a clinical continuum. These diseases, in order of increasing severity, are familial cold autoinflammatory syndrome (FCAS), Muckle-Wells syndrome (MWS), and chronic infantile cutaneous neurological articular syndrome (CINCA; also called neonatal-onset multisystem inflammatory disease, NOMID), and all have been shown to result from gain-of-function mutations in the NLRP3 gene, which leads to increased secretion of IL-I beta. NLRP3 has also been implicated in a number of autoinflammatory diseases, including pyogenic arthritis, pyoderma gangrenosum and acne (PAPA), Sweet's syndrome, chronic nonbacterial osteomyelitis (CNO), and acne vulgaris (Cook et al, Eur J. Immunol., 2010, 40, 595-653). A number of autoimmune diseases have been shown to involve NLRP3 including, in particular, multiple sclerosis, type-1 diabetes (TlD), psoriasis, rheumatoid arthritis (RA), Behcet's disease, Schnitzler syndrome, macrophage activation syndrome (Braddock et al. Nat. Rev. Drug Disc. 2004, 3, 1-10; Inoue et al, Immunology, 2013, 139, 11-18, Coll et al, Nat. Med. 2015, 21(3), 248-55; Scott et al, Clin. Exp. Rheumatol. 2016, 34(1), 88-93), systemic lupus erythematosus and its complications such as lupus nephritis (Lu et al, J. Immunol., 2017, 198(3), 1119-29), and systemic sclerosis (Artlett et al, Arthritis Rheum. 2011, 63(11), 3563-74). NLRP3 has also been shown to play a role in a number of lung diseases including chronic obstructive pulmonary disorder (COPD), asthma (including steroid resistant asthma), asbestosis, and silicosis (De Nardo et al, Am. J. Pathol., 2014, 184: 42-54; Kim et al. Am. J. Respir Crit Care Med, 2017, 196(3), 283-97). NLRP3 has also been suggested to have a role in a number of central nervous system conditions, including Multiple Sclerosis (MS), Parkinson's disease (PD), Alzheimer's disease (AD), dementia, Huntington's disease, cerebral malaria, brain injury from pneumococcal meningitis (Walsh et al, Nature Reviews, 2014, 15, 84-97; and Dempsey et al. Brain. Behav. Immun. 2017, 61, 306-16), intracranial aneurysms (Zhang et al. J. Stroke and Cerebrovascular Dis., 2015, 24, 5, 972-9), and traumatic brain injury (Ismael et al. J. Neurotrauma., 2018, 35(11), 1294-1303). NRLP3 activity has also been shown to be involved in various metabolic diseases including type 2 diabetes (T2D) and its organ-specific complications, atherosclerosis, obesity, gout, pseudo-gout, metabolic syndrome (Wen et al, Nature Immunology, 2012, 13, 352-357; Duewell et al, Nature, 2010, 464, 1357-1361; Strowig et al, Nature, 2014, 481, 278-286), and non-alcoholic steatohepatitis (Mridha et al. J. Hepatol. 2017, 66(5), 1037-46). NLRP3 is also suggested to play a key pathological role in the development and progression of several skeletal muscle diseases, e.g. muscle atrophy, inherited and acquired myopathies (Dubussion et al. Cells 2021, 10(11):3023). A role for NLRP3 via IL-I beta has also been suggested in atherosclerosis, myocardial infarction (van Hout et al. Eur Heart J. 2017, 38(11), 828-36), heart failure (Sano et al. J. Am. Coll. Cardiol. 2018, 71(8), 875-66), aortic aneurysm and dissection (Wu et al. Arterioscler Thromb. Vase. Biol., 2017, 37(4), 694-706), and other cardiovascular events (Ridker et al, N. Engl. J. Med, 2017, 377(12), 1119-31). Other diseases in which NLRP3 has been shown to be involved include: ocular diseases such as both wet and dry age-related macular degeneration (Doyle et al. Nature Medicine, 2012, 18, 791-798; Tarallo et al. Cell 2012, 149(4), 847-59), diabetic retinopathy (Loukovaara et al. Acta Ophthalmol., 2017, 95(8), 803-8), non-infectious uveitis and optic nerve damage (Puyang et al. Sci. Rep. 2016, 6, 20998); liver diseases including non-alcoholic steatohepatitis (NASH) and acute alcoholic hepatitis (Henao-Meija et al, Nature, 2012, 482, 179-185); inflammatory reactions in the lung and skin (Primiano et al. J. Immunol. 2016, 197(6), 2421-33) including contact hypersensitivity (such as bullous pemphigoid (Fang et al. J Dermatol Sci. 2016, 83(2),116-23)), atopic dermatitis (Niebuhr et al. Allergy, 2014, 69(8), 1058-67), Hidradenitis suppurativa (Alikhan et al. J. Am. Acad. Dermatol., 2009, 60(4), 539-61), and sarcoidosis (Jager et al. Am. J. Respir Crit. Care Med., 2015, 191, A5816); inflammatory reactions in the joints (Braddock et al, Nat. Rev. Drug Disc, 2004, 3, 1-10); amyotrophic lateral sclerosis (Gugliandolo et al. Int. J. Mol. Sci., 2018, 19(7), E1992); cystic fibrosis (Iarmitti et al. Nat. Commun., 2016,7, 10791); stroke (Walsh et al, Nature Reviews, 2014, 15, 84-97); chronic kidney disease (Granata et al. PLoS One 2015, 10(3), eoi22272); and inflammatory bowel diseases including ulcerative colitis and Crohn's disease (Braddock et al, Nat. Rev. Drug Disc, 2004, 3, 1-10; Neudecker et al. J. Exp. Med. 2017, 214(6), 1737-52; Lazaridis et al. Dig. Dis. Sci. 2017, 62(9), 2348-56). The NLRP3 inflammasome has been found to be activated in response to oxidative stress. NLRP3 has also been shown to be involved in inflammatory hyperalgesia (Dolunay et al, Inflammation, 2017, 40, 366-86). US application US20200361898 in incorporated herein by reference.

### Definitions

To assist in understanding the scope of the compounds of Formulae I-XI or a form thereof described herein, the following Specific Examples are included. The experiments relating to the compounds of Formulae I-XI or a form thereof described herein should not, of course, be construed as specifically limiting the scope of the compounds of Formulae I-XI or a form thereof described herein and such variations of the compounds of Formulae I-XI or a form thereof as described herein, now known or later developed, which would be within the purview of one skilled in the art are considered to fall within the scope as described herein and hereinafter claimed.

Other than in the working examples, unless indicated to the contrary, all numbers expressing quantities of ingredients, reaction conditions, experimental data, and so forth used in the specification and claims are to be understood as being modified by the term “about”. Accordingly, all such numbers represent approximations that may vary depending upon the desired properties sought to be obtained by a reaction or as a result of variable experimental conditions. Therefore, within an expected range of experimental reproducibility, the term “about” in the context of the resulting data, refers to a range for data provided that may vary according to a standard deviation from the mean. As well, for experimental results provided, the resulting data may be rounded up or down to present data consistently, without loss of significant figures. At the very least, and not as an attempt to limit the application of the doctrine of equivalents to the scope of the claims, each numerical parameter should be construed in light of the number of significant digits and ordinary rounding techniques.

While the numerical ranges and parameters setting forth the characterization of the compounds of Formulae I-XI or a form thereof described herein are approximations, the numerical values set forth in the working examples are reported as precisely as possible. Any numerical value, however, inherently contains certain errors necessarily resulting from the standard deviation found in their respective testing measurements.

The compounds of Formulae I-XI or a form thereof provided herein are described in more detail with reference to the following non-limiting examples, which are offered to more fully illustrate the scope of the compounds of Formulae I-XI or a form thereof described herein, but are not to be construed as limiting the scope thereof. The examples illustrate the preparation of compounds of Formulae I-XI or a form thereof described herein, and the testing of these compounds of Formulae I-XI or a form thereof in vitro and/or in vivo. Those of skill in the art will understand that the synthesis techniques described in these examples represent techniques that fall within the practice of those having ordinary skill in the chemical arts, and as such constitute preferred modes for the practice thereof. However, it should be appreciated that those having skill in the art should, in light of the present disclosure, appreciate that many changes can be made in the specific methods that are disclosed herein while still obtaining a like or similar result without departing from the spirit and scope described herein.

In certain embodiments described herein, the compound of Formulae I-XI or a form thereof is isolated for use.

As used herein, the term “isolated” means the physical state of a compound of Formulae I-XI or a form thereof after being isolated and/or separated and/or purified from a synthetic process (e.g., from a reaction mixture) or natural source or combination thereof according to an isolation, separation or purification process or processes described herein or which are well known to the skilled artisan (e.g., chromatography, recrystallization and the like) in sufficient purity to be characterizable by standard analytical techniques described herein or well known to the skilled artisan.

As used herein, the term “protected” means that a functional group on a compound of Formulae I-XI or a form thereof is in a form modified to preclude undesired side reactions of the functional group when the compound is subjected to a reaction. Suitable protecting groups will be recognized by those with ordinary skill in the art as well as by reference to standard textbooks such as, for example, T. W. Greene et al, Protective Groups in Organic Synthesis (2007), Wiley, New York.

Prodrugs and solvates of the compounds of Formulae I-XI or a form thereof described herein are also contemplated.

As used herein, the term “prodrug” means that a functional group on a compound of Formulae I-XI is in a form (e.g., acting as an active or inactive drug precursor) that is transformed in vivo to yield an active or more active compound of Formulae I-XI or a form thereof. The transformation may occur by various mechanisms (e.g., by metabolic and/or nonmetabolic chemical processes), such as, for example, by hydrolysis and/or metabolism in blood, liver and/or other organs and tissues. A discussion of the use of prodrugs is provided by V. J. Stella, et. al., “Biotechnology: Pharmaceutical Aspects, Prodrugs: Challenges and Rewards,” American Association of Pharmaceutical Scientists and Springer Press, 2007.

In one example, when a compound of Formulae I-XI or a form thereof contains a carboxylic acid functional group, a prodrug can comprise an ester formed by the replacement of the hydrogen atom of the acid group with a functional group such as alkyl and the like. In another example, when a compound of Formulae I-XI or a form thereof contains an alcohol functional group, a prodrug can be formed by the replacement of the hydrogen atom of the alcohol group with a functional group such as alkyl or carbonyloxy and the like. In another example, when a compound of Formulae I-XI or a form thereof contains an amine functional group, a prodrug can be formed by the replacement of one or more amine hydrogen atoms with a functional group such as alkyl or substituted carbonyl.

Pharmaceutically acceptable prodrugs of compounds of Formulae I-XI or a form thereof include those compounds substituted with one or more of the following groups: carboxylic acid esters, sulfonate esters, amino acid esters, phosphonate esters (e.g., a phosphoramidic acid used to derive a phosphoramidic acid) and mono-, di- or triphosphate esters further substituted with alkyl, where appropriate. As described herein, it is understood by a person of ordinary skill in the art that one or more of such substituents may be used to provide a compound of Formulae I-XI or a form thereof as a prodrug.

The compounds of Formulae I-XI or a form thereof can form salts, which are intended to be included within the scope of this description. Reference to a compound of Formulae I-XI or a form thereof herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formulae I-XI or a form thereof contains both a basic moiety, such as, but not limited to a pyridine or imidazole, and an acidic moiety, such as, but not limited to a carboxylic acid, zwitterions (“inner salts”) may be formed and are included within the term “salt(s)” as used herein.

The term “pharmaceutically acceptable salt(s)”, as used herein, means those salts of compounds of Formulae I-XI or a form thereof described herein that are safe and effective (i.e., non-toxic, physiologically acceptable) for use in mammals and that possess biological activity, although other salts are also useful. Salts of the compounds of the Formulae I-XI may be formed, for example, by reacting a compound of Formulae I-XI with an amount of acid or base, such as an equivalent amount, in a medium such as one in which the salt precipitates or in an aqueous medium followed by lyophilization.

Pharmaceutically acceptable salts include one or more salts of acidic or basic groups present in compounds of Formulae I-XI or a form thereof described herein. Embodiments of acid addition salts include, and are not limited to, acetate, acid phosphate, ascorbate, benzoate, benzenesulfonate, bisulfate, bitartrate, borate, butyrate, chloride, citrate, camphorate, camphorsulfonate, ethanesulfonate, formate, fumarate, gentisinate, gluconate, glucaronate, glutamate, hydrobromide, hydrochloride, dihydrochloride, hydroiodide, isonicotinate, lactate, maleate, methanesulfonate, naphthalenesulfonate, nitrate, oxalate, pamoate, pantothenate, phosphate, propionate, saccharate, salicylate, succinate, sulfate, tartrate, thiocyanate, toluenesulfonate (also known as tosylate), trifluoroacetate, trifluoroacetic acid salt and the like. One or more embodiments of acid addition salts include chloride, hydrochloride, dihydrochloride, trihydrochloride, hydrobromide, acetate, diacetate, methanesulfonate, sulfate, trifluoroacetate, trifluoroacetic acid salt and the like. More particular embodiments include a chloride, hydrochloride, dihydrochloride, hydrobromide, methanesulfonate, sulfate, trifluoroacetate, trifluoroacetic acid salt and the like.

In certain embodiments of the compounds of Formulae I-XI or a form thereof described herein, the compound is isolated as a salt form, wherein the compound is conjugated with the salt in a ratio represented as, in a non-limiting example, “compound:salt (A:B),” wherein “A” and “B” represent the equivalents of compound to salt in the isolated form.

Additionally, acids which are considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33, 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Suitable basic salts include, but are not limited to, aluminum, ammonium, calcium, lithium, magnesium, potassium, sodium, zinc, and diethanolamine salts. Certain compounds of Formulae I-XI or a form thereof described herein can also form pharmaceutically acceptable salts with organic bases (for example, organic amines) such as, but not limited to, dicyclohexylamines, tert-butyl amines and the like, and with various amino acids such as, but not limited to, arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be included within the scope of pharmaceutically acceptable salts as described herein. In addition, all such acid and base salts are considered equivalent to the free forms of the corresponding compounds for purposes of this description.

Compounds of Formulae I-XI, and forms thereof, may further exist in a tautomeric form. All such tautomeric forms are contemplated and intended to be included within the scope of the compounds of Formulae I-XI or a form thereof as described herein.

The compounds of Formulae I-XI or a form thereof may contain asymmetric or chiral centers, and, therefore, may exist in different stereoisomeric forms. The present description is intended to include all stereoisomeric forms of the compounds of Formulae I-XI as well as mixtures thereof, including racemic mixtures.

The compounds of Formulae I-XI or a form thereof described herein may include one or more chiral centers, and as such may exist as racemic mixtures (R S) or as substantially pure enantiomers and diastereomers. The compounds may also exist as substantially pure (R) or (S) enantiomers (when one chiral center is present). In one embodiment, the compounds of Formulae I-XI or a form thereof described herein are (S) isomers and may exist as enantiomerically pure compositions substantially comprising only the (S) isomer. In another embodiment, the compounds of Formulae I-XI or a form thereof described herein are (R) isomers and may exist as enantiomerically pure compositions substantially comprising only the (R) isomer. As one of skill in the art will recognize, when more than one chiral center is present, the compounds of Formulae I-XI or a form thereof described herein may also exist as a (R,R), (R,S), (S,R) or (S,S) isomer, as defined by IUPAC Nomenclature Recommendations.

As used herein, the term “substantially pure” refers to compounds of Formulae I-XI or a form thereof consisting substantially of a single isomer in an amount greater than or equal to 90%, in an amount greater than or equal to 92%, in an amount greater than or equal to 95%, in an amount greater than or equal to 98%, in an amount greater than or equal to 99%, or in an amount equal to 100% of the single isomer.

In one aspect of the description, a compound of Formulae I-XI or a form thereof is a substantially pure (S) enantiomer present in an amount greater than or equal to 90%, in an amount greater than or equal to 92%, in an amount greater than or equal to 95%, in an amount greater than or equal to 98%, in an amount greater than or equal to 99%, or in an amount equal to 100%.

In one aspect of the description, a compound of Formulae I-XI or a form thereof is a substantially pure (R) enantiomer present in an amount greater than or equal to 90%, in an amount greater than or equal to 92%, in an amount greater than or equal to 95%, in an amount greater than or equal to 98%, in an amount greater than or equal to 99%, or in an amount equal to 100%.

As used herein, the term “racemate” refers to any mixture of isometric forms that are not “enantiomerically pure”, including mixtures such as, without limitation, in a ratio of about 50/50, about 60/40, about 70/30, or about 80/20, about 85/15 or about 90/10.

In addition, the compounds of Formulae I-XI or a form thereof described herein embrace all geometric and positional isomers. For example, if a compound of Formulae I-XI or a form thereof incorporates a double bond or a fused ring, both the cis- and trans-forms, as well as mixtures thereof, are embraced within the scope of the compounds of Formulae I-XI or a form thereof described herein.

Diastereomeric mixtures can be separated into their individual diastereomers on the basis of their physical chemical differences by methods well known to those skilled in the art, such as, for example, by chromatography and/or fractional crystallization. Enantiomers can be separated by use of a chiral HPLC column or other chromatographic methods known to those skilled in the art.

Enantiomers can also be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers.

All stereoisomers (for example, geometric isomers, optical isomers and the like) of the present compounds of Formulae I-XI or a form thereof (including salts, solvates, esters and prodrugs and transformed prodrugs thereof), which may exist due to asymmetric carbons on various substituents, including enantiomeric forms (which may exist even in the absence of asymmetric carbons), rotameric forms, atropisomers, diastereomeric and regioisomeric forms, are contemplated within the scope of the description herein. Individual stereoisomers of the compounds of Formulae I-XI or a form thereof described herein may, for example, be substantially free of other isomers, or may be present in a racemic mixture, as described supra.

The use of the terms “salt,” “solvate,” “ester,” “prodrug” and the like, is intended to apply equally to the salt, solvate, ester and prodrug of enantiomers, stereoisomers, rotamers, tautomers, positional isomers, racemates, isotopologues or prodrugs of the instant compounds.

One or more compounds of Formulae I-XI or a form thereof described herein may exist in unsolvated as well as solvated forms with pharmaceutically acceptable solvents such as water, ethanol, and the like, and the description herein is intended to embrace both solvated and unsolvated forms.

As used herein, the term “solvate” means a physical association of a compound of Formulae I-XI or a form thereof described herein with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. As used herein, “solvate” encompasses both solution-phase and isolatable solvates. Non-limiting examples of suitable solvates include ethanolates, methanolates, and the like.

One or more compounds of Formulae I-XI or a form thereof described herein may optionally be converted to a solvate. Preparation of solvates is generally known. A typical, non-limiting process involves dissolving a compound of Formulae I-XI or a form thereof in a desired amount of the desired solvent (organic or water or mixtures thereof) at a higher than ambient temperature, and cooling the solution at a rate sufficient to form crystals which are then isolated by standard methods. Analytical techniques such as, for example infrared spectroscopy, show the presence of the solvent (or water) in the crystals as a solvate (or hydrate).

As used herein, the term “hydrate” means a solvate wherein the solvent molecule is water.

As used herein, the term “isotope enriched” means a compounds of Formulae I-XI or a form thereof which are identical to those recited herein, but for the fact that one or more atoms are replaced by an atom having an atomic mass or mass number different from the atomic mass or mass number usually found in nature. Examples of isotopes that can be incorporated into compounds of Formula (I) or a form thereof described herein include isotopes of hydrogen, carbon, nitrogen, oxygen, phosphorus, fluorine and chlorine, such as H², H³, C¹³, C¹⁴, N¹⁵, O¹⁸, O¹⁷, P³¹, P³², S³⁵, F¹⁸, Cl³⁵ and Cl³⁶, respectively, each of which is also within the scope of this description.

Polymorphic crystalline and amorphous forms of the compounds of Formulae I-XI or a form thereof, and of the salts, solvates, esters and prodrugs of the compounds of Formulae I-XI or a form thereof, are further intended to be included in the scope of the compounds of Formulae I-XI or a form thereof described herein.

### Terminology

The chemical terms used above and throughout the description herein, unless specifically defined otherwise, shall be understood by one of ordinary skill in the art to have the following indicated meanings.

As used herein, the term “C₁₋₈alkyl” refers to saturated hydrocarbon radicals having from one to eight carbon atoms in a straight or branched chain configuration, including, without limitation, methyl, ethyl, n-propyl, isopropyl, n-butyl, isobutyl, sec-butyl, tert-butyl, n-pentyl, n-hexyl, n-heptyl, n-octyl and the like. In some embodiments, C₁₋₈alkyl includes C₁₋₆alkyl, C₁₋₄alkyl and the like. A C₁₋₈alkyl radical may be optionally substituted where allowed by available valences.

As used herein, the term “aryl” refers to a monocyclic, bicyclic or polycyclic aromatic carbon atom ring structure radical, including, without limitation, phenyl, naphthyl (also referred to as naphthalenyl), anthracenyl, fluorenyl, azulenyl, phenanthrenyl and the like. An aryl radical may be optionally substituted where allowed by available valences.

As used herein, the term “heteroaryl” refers to a monocyclic, bicyclic or polycyclic aromatic carbon atom ring structure radical in which one or more carbon atom ring members have been replaced, where allowed by structural stability, with one or more heteroatoms, such as an O, S or N atom, including, without limitation, furanyl, thienyl (also referred to as thiophenyl), pyrrolyl, pyrazolyl (also referred to as 1H-pyrazolyl), imidazolyl (also referred to as 1H-imidazolyl), isoxazolyl (also referred to as 1,2-oxazolyl), isothiazolyl, oxazolyl, thiazolyl, triazolyl, oxadiazolyl, thiadiazolyl, tetrazolyl, pyranyl, thiopyranyl, pyridinyl (also referred to as pyridyl), pyrimidinyl, pyrazinyl, pyridazinyl, triazinyl, indolyl (also referred to as 1H-indolyl), azaindolyl, indazolyl (also referred to as 2H-indazolyl), azaindazolyl, isoindolyl, indolizinyl, benzofuranyl, benzothienyl, benzimidazolyl (also referred to as 1H-benzimidazolyl), benzothiazolyl, benzoxazolyl, imidazo[2,1-b][1,3]thiazolyl, pyrazolo[1,5-a]pyridinyl, pyrazolo[1,5-c]pyrimidinyl, imidazo[1,2-a]pyridinyl, 1H-imidazo[4,5-b]pyridinyl, 1H-imidazo[4,5-c]pyridinyl, imidazo[1,2-a]pyrazinyl, imidazo[1,2-a]pyrimidinyl, 7H-purinyl, 9H-purinyl, quinolinyl, isoquinolinyl, quinazolinyl, quinoxalinyl, acridinyl and the like and associated homologs and regioisomers thereof. A heteroaryl radical may be optionally substituted on a carbon or nitrogen atom ring member where allowed by available valences.

As used herein, the term “heterocyclyl” refers to a saturated or partially unsaturated monocyclic, bicyclic or polycyclic carbon atom ring structure radical in which one or more carbon atom ring members have been replaced, where allowed by structural stability, with a heteroatom, such as an O, S or N atom, including, without limitation, oxiranyl, oxetanyl, azetidinyl, dihydrofuranyl, tetrahydrofuranyl, dihydrothienyl, tetrahydrothienyl, pyrrolinyl, pyrrolidinyl, dihydropyrazolyl, pyrazolinyl, pyrazolidinyl, dihydroimidazolyl, imidazolinyl, imidazolidinyl, isoxazolinyl, isoxazolidinyl, isothiazolinyl, isothiazolidinyl, oxazolinyl, oxazolidinyl, thiazolinyl, thiazolidinyl, triazolinyl, triazolidinyl, oxadiazolinyl, oxadiazolidinyl, thiadiazolinyl, thiadiazolidinyl, tetrazolinyl, tetrazolidinyl, 1,3-dioxolanyl, dihydro-2H-pyranyl, tetrahydro-2H-pyranyl, dihydro-pyridinyl, tetrahydro-pyridinyl, dihydro-pyrimidinyl, tetrahydro-pyrimidinyl, dihydro-pyrazinyl, tetrahydro-pyrazinyl, dihydro-pyridazinyl, tetrahydro-pyridazinyl, piperazinyl, piperidinyl, morpholinyl, thiomorpholinyl, dihydro-triazinyl, tetrahydro-triazinyl, hexahydro-triazinyl, 1,4-diazepanyl, dihydro-indolyl, indolinyl, tetrahydro-indolyl, dihydro-indazolyl, tetrahydro-indazolyl, dihydro-isoindolyl, dihydro-benzofuranyl, tetrahydro-benzofuranyl, dihydro-benzothienyl, tetrahydro-benzothienyl, dihydro-benzoimidazolyl, tetrahydro-benzoimidazolyl, dihydro-benzooxazolyl, tetrahydro-benzooxazolyl, dihydro-benzooxazinyl, tetrahydro-benzooxazinyl, benzo[1,3]dioxolyl (also referred to as 1,3-benzodioxolyl), benzo[1,4]dioxanyl (also referred to as 1,4-benzodioxanyl or 2,3-dihydro-1,4-benzodioxinyl), benzo[1,4]dioxinyl (also referred to as 1,4-benzodioxinyl), 4,5,6,7-tetrahydro-2H-indazolyl, 5,6,7,8-tetrahydroimidazo[1,2-a]pyridinyl, 4,5,6,7-tetrahydro-3H-imidazo[4,5-c]pyridinyl, dihydro-purinyl, tetrahydro-purinyl, dihydro-quinolinyl, tetrahydro-quinolinyl, dihydro-isoquinolinyl, tetrahydro-isoquinolinyl, dihydro-quinazolinyl, tetrahydro-quinazolinyl, dihydro-quinoxalinyl, tetrahydro-quinoxalinyl and the like and associated homologs thereof. A heterocyclyl radical may be optionally substituted on a carbon or nitrogen atom ring member where allowed by available valences.

As used herein, the term “halo” or “halogen” generally refers to a halogen atom radical, including fluoro, chloro, bromo and iodo.

As used herein, the term “C₁₋₄alkoxy-C₁₋₄alkyl” refers to a radical of the formula: —C₁₋₄alkyl-O—C₁₋₄alkyl.

As used herein, the term “C₁₋₄alkoxy-carbonyl” refers to a radical of the formula: —C(O)—O—C₁₋₄alkyl.

As used herein, the term “C₁₋₄alkoxy-carbonyl-amino” refers to a radical of the formula: —NH—C(O)—O—C₁₋₄alkyl.

As used herein, the term “C₁₋₄alkyl-amino” refers to a radical of the formula: —NH—C₁₋₄alkyl.

As used herein, the term “(C₁₋₄alkyl)₂-amino” refers to a radical of the formula: —N(C₁₋₄alkyl)₂.

As used herein, the term “C₁₋₄alkyl-amino-C₁₋₄alkoxy” refers to a radical of the formula: —O—C₁₋₄alkyl-NH—C₁₋₄alkyl.

As used herein, the term “(C₁₋₄alkyl)₂-amino-C₁₋₄alkoxy” refers to a radical of the formula: —O—C₁₋₄alkyl-N(C₁₋₄alkyl)₂.

As used herein, the term “C₁₋₄alkyl-amino-C₁₋₄alkyl” refers to a radical of the formula: —C₁₋₄alkyl-NH—C₁₋₄alkyl.

As used herein, the term “(C₁₋₄alkyl)₂-amino-C₁₋₄alkyl” refers to a radical of the formula: —C₁₋₄alkyl-N(C₁₋₄alkyl)₂.

As used herein, the term “C₁₋₄alkyl-carbonyl” refers to a radical of the formula: —C(O)—C₁₋₄alkyl.

As used herein, the term “C₁₋₄alkyl-carbonyl-amino” refers to a radical of the formula: —NH—C(O)—C₁₋₄alkyl.

As used herein, the term “C₁₋₄alkyl-thio” refers to a radical of the formula: —S—C₁₋₄alkyl.

As used herein, the term “amino-C₂₋₈alkenyl” refers to a radical of the formula: —C₂₋₈alkenyl-NH₂.

As used herein, the term “amino-C₁₋₄alkoxy” refers to a radical of the formula: —O—C₁₋₄alkyl-NH₂.

As used herein, the term “amino-C₁₋₄alkyl” refers to a radical of the formula: —C₁₋₄alkyl-NH₂.

As used herein, the term “amino-C₁₋₄alkyl-amino” refers to a radical of the formula: —NH—C₁₋₄alkyl-NH₂.

As used herein, the term “(amino-C₁₋₄alkyl)₂-amino” refers to a radical of the formula: —N(C₁₋₄alkyl-NH₂)₂.

As used herein, the term “(amino-C₁₋₄alkyl)(C₁₋₄alkyl)amino” refers to a radical of the formula: —N(C₁₋₄alkyl)(C₁₋₄alkyl-NH₂).

As used herein, the term “amino-C₂₋₈alkynyl” refers to a radical of the formula: —C₂₋₈alkynyl-NH₂.

As used herein, the term “aryl-C₁₋₄alkoxy-carbonyl” refers to a radical of the formula: —C(O)—O—C₁₋₄alkyl-aryl.

As used herein, the term “aryl-C₁₋₄alkyl” refers to a radical of the formula: —C₁₋₄alkyl-aryl.

As used herein, the term “aryl-C₁₋₄alkyl-amino” refers to a radical of the formula: —NH—C₁₋₄alkyl-aryl.

As used herein, the term “(aryl-C₁₋₄alkyl)₂-amino” refers to a radical of the formula: —N(C₁₋₄alkyl-aryl)₂.

As used herein, the term “aryl-amino” refers to a radical of the formula: —NH-aryl.

As used herein, the term “aryl-amino-carbonyl” refers to a radical of the formula: —C(O)—NH-aryl.

As used herein, the term “benzoxy-carbonyl” refers to a radical of the formula: —C(O)—O—CH₂-phenyl.

As used herein, the term “C₃₋₁₄cycloalkyl-C₁₋₄alkyl” refers to a radical of the formula: —C₁₋₄alkyl-C₃₋₁₄cycloalkyl.

As used herein, the term “C₃₋₁₄cycloalkyl-amino” refers to a radical of the formula: —NH—C₃₋₁₄cycloalkyl.

As used herein, the term “C₃₋₁₄cycloalkyl-oxy” refers to a radical of the formula: —O—C₃₋₁₄cycloalkyl.

As used herein, the term “deutero-C₁₋₄alkyl,” refers to a radical of the formula: —C₁₋₄alkyl-deutero, wherein C₁₋₄alkyl is partially or completely substituted with one or more deuterium atoms where allowed by available valences.

As used herein, the term “halo-C₁₋₄alkoxy” refers to a radical of the formula: —O—C₁₋₄alkyl-halo, wherein C₁₋₄alkyl is partially or completely substituted with one or more halogen atoms where allowed by available valences.

As used herein, the term “halo-C₁₋₄alkyl” refers to a radical of the formula: —C₁₋₄alkyl-halo, wherein C₁₋₄alkyl is partially or completely substituted with one or more halogen atoms where allowed by available valences.

As used herein, the term “halo-C₁₋₄alkyl-amino” refers to a radical of the formula: —NH—C₁₋₄alkyl-halo.

As used herein, the term “(halo-C₁₋₄alkyl)(C₁₋₄alkyl)amino” refers to a radical of the formula: —N(C₁₋₄alkyl)(C₁₋₄alkyl-halo).

As used herein, the term “(halo-C₁₋₄alkyl)₂-amino” refers to a radical of the formula: —N(C₁₋₄alkyl-halo)₂.

As used herein, the term “heteroaryl-C₁₋₄alkoxy” refers to a radical of the formula: —O—C₁₋₄alkyl-heteroaryl.

As used herein, the term “heteroaryl-C₁₋₄alkyl” refers to a radical of the formula: —C₁₋₄alkyl-heteroaryl.

As used herein, the term “heteroaryl-amino” refers to a radical of the formula: —NH-heteroaryl.

As used herein, the term “heterocyclyl-C₁₋₄alkoxy” refers to a radical of the formula: —O—C₁₋₄alkyl-heterocyclyl.

As used herein, the term “heterocyclyl-C₁₋₄alkyl” refers to a radical of the formula: —C₁₋₄alkyl-heterocyclyl.

As used herein, the term “heterocyclyl-C₁₋₄alkyl-amino” refers to a radical of the formula: —NH—C₁₋₄alkyl-heterocyclyl.

As used herein, the term “(heterocyclyl-C₁₋₄alkyl)₂-amino” refers to a radical of the formula: —N(C₁₋₄alkyl-heterocyclyl)₂.

As used herein, the term “heterocyclyl-amino” refers to a radical of the formula: —NH-heterocyclyl.

As used herein, the term “(heterocyclyl)(C₁₋₄alkyl)amino” refers to a radical of the formula: —N(C₁₋₄alkyl)(heterocyclyl).

As used herein, the term “heterocyclyl-amino-C₁₋₄alkyl” refers to a radical of the formula: —C₁₋₄alkyl-NH-heterocyclyl.

As used herein, the term “heterocyclyl-carbonyl” refers to a radical of the formula: —C(O)-heterocyclyl.

As used herein, the term “heterocyclyl-carbonyl-oxy” refers to a radical of the formula: —O—C(O)-heterocyclyl.

As used herein, the term “heterocyclyl-oxy” refers to a radical of the formula: —O-heterocyclyl.

As used herein, the term “hydroxy” refers to a radical of the formula: —OH.

As used herein, the term “hydroxy-C₁₋₄alkoxy-C₁₋₄alkyl” refers to a radical of the formula: —C₁₋₄alkyl-O—C₁₋₄alkyl-OH.

As used herein, the term “hydroxy-C₁₋₄alkyl” refers to a radical of the formula: —C₁₋₄alkyl-OH, wherein C₁₋₄alkyl is partially or completely substituted with one or more hydroxy radicals where allowed by available valences.

As used herein, the term “hydroxy-C₁₋₄alkyl-amino” refers to a radical of the formula: —NH—C₁₋₄alkyl-OH.

As used herein, the term “(hydroxy-C₁₋₄alkyl)₂-amino” refers to a radical of the formula: —N(C₁₋₄alkyl-OH)₂.

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances one or more substituents having a double bond (e.g., “oxo” or “═O”) as the point of attachment may be described, shown or listed herein within a substituent group, wherein the structure may only show a single bond as the point of attachment to the core structure of Formulae I-XI. A person of ordinary skill in the art would understand that, while only a single bond is shown, a double bond is intended for those substituents.

As used herein, the term “and the like,” with reference to the definitions of chemical terms provided herein, means that variations in chemical structures that could be expected by one skilled in the art include, without limitation, isomers (including chain, branching or positional structural isomers), hydration of ring systems (including saturation or partial unsaturation of monocyclic, bicyclic or polycyclic ring structures) and all other variations where allowed by available valences which result in a stable compound.

As used herein, the term “substituent” means positional variables on the atoms of a core molecule that are attached at a designated atom position, replacing one or more hydrogen atoms on the designated atom, provided that the atom of attachment does not exceed the available valence or shared valences, such that the substitution results in a stable compound. Accordingly, combinations of substituents and/or variables are permissible only if such combinations result in stable compounds. It should also be noted that any carbon as well as heteroatom with a valence level that appears to be unsatisfied as described or shown herein is assumed to have a sufficient number of hydrogen atom(s) to satisfy the valences described or shown.

For the purposes of this description, where one or more substituent variables for a compound of Formulae I-XI encompass functionalities incorporated into a compound of Formulae I-XI, each functionality appearing at any location within the disclosed compound may be independently selected, and as appropriate, independently and/or optionally substituted.

As used herein, the terms “independently selected,” or “each selected” refer to functional variables in a substituent list that may be attached more than once on the structure of a core molecule, where the pattern of substitution at each occurrence is independent of the pattern at any other occurrence. Further, the use of a generic substituent on a core structure for a compound provided herein is understood to include the replacement of the generic substituent with specie substituents that are included within the particular genus, e.g., aryl may be independently replaced with phenyl or naphthalenyl (also referred to as naphthyl) and the like, such that the resulting compound is intended to be included within the scope of the compounds described herein.

As used herein, the term “each instance of” or “each variable is independently” when used in a phrase such as “ . . . aryl, aryl-C₁₋₈alkyl, heterocyclyl and heterocyclyl-C₁₋₈alkyl, wherein each instance of aryl and heterocyclyl is optionally substituted with one or two substituents . . . ” is intended to include optional, independent substitution on each of the aryl and heterocyclyl rings and on the aryl and heterocyclyl portions of aryl-C₁₋₈alkyl and heterocyclyl-C₁₋₈alkyl.

As used herein, the term “optionally substituted” means that the specified substituent variables, groups, radicals or moieties represent the scope of the genus and may be independently chosen as needed to replace one or more hydrogen atoms on the designated atom of attachment of a core molecule.

As used herein, the terms “stable compound” or “stable structure” means a compound that is sufficiently robust to be isolated to a useful degree of purity from a reaction mixture and Formulations thereof into an efficacious therapeutic agent.

As used herein, the terms “subject” and “patient” are used interchangeably to refer to an animal or any living organism having sensation and the power of voluntary movement, and which requires for its existence oxygen and organic food. Nonlimiting examples include members of the human, equine, porcine, bovine, rattus, murine, canine and feline species. In some embodiments, the subject is a mammal or a warm-blooded vertebrate animal. In certain embodiments, the subject is a non-human animal. In specific embodiments, the subject is a human.

Compound names provided herein were obtained using ACD Labs Index Name software provided by ACD Labs and/or ChemDraw Ultra software provided by CambridgeSoft®. When the compound name disclosed herein conflicts with the structure depicted, the structure shown will supercede the use of the name to define the compound intended. Nomenclature for substituent radicals defined herein may differ slightly from the chemical name from which they are derived; one skilled in the art will recognize that the definition of the substituent radical is intended to include the radical as found in the chemical name.

### Dosage and Administration

The compounds of the present invention may be formulated in a wide variety of oral administration dosage forms and carriers. Oral administration can be in the form of tablets, coated tablets, dragées, hard and soft gelatine capsules, solutions, emulsions, syrups, or suspensions. Compounds of the present invention are efficacious when administered by other routes of administration including continuous (intravenous drip) topical parenteral, intramuscular, intravenous, subcutaneous, transdermal (which may include a penetration enhancement agent), buccal, nasal, inhalation and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulating agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents, formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polyactic acid.

Suitable formulations along with pharmaceutical carriers, diluents and expcipients are described in Remington: The Science and Practice of Pharmacy 1995, edited by E. W. Martin, Mack Publishing Company, 19th edition, Easton, Pennsylvania. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

In general, the nomenclature used in this Application is based on AUTONOM™ v.4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

The reagents and solvents were used as purchased (from a variety of vendors), except where noted. Where applicable, the term “Celite” is used as shown in the following examples to represent the tradename CELITE© (brand of diatomaceous earth). Where applicable, chromatographic separations were performed using techniques and equipment commonly available such as, for example, by using an ISCO CombiFlash© Rf system. Where applicable, NMR spectra were obtained using techniques and equipment commonly available such as, for example, by using a Bruker Avance III⁵⁰⁰ spectrometer with deuterated solvents such as, for example, DMSO-d₆ or residual solvent as standard. Where applicable, TLC analysis was performed using techniques and equipment commonly available such as, for example, by using Aldrich 254 nm glass-backed plates (60 Å, 250 μm), visualized using UV and I₂ stains. Where applicable, ESI mass spectra were obtained using techniques and equipment commonly available such as, for example, by using an ACQUITY UPLC© System, with values shown as [M+H]⁺ or [M−H]⁻, unless otherwise indicated. Where applicable, the structure of the product was obtained via a 2D NOESY (Nuclear Overhauser SpectroscopY) experiment.

### Chemical Definitions

As used herein, the term “C₁₋₄alkyl” generally refers to saturated hydrocarbon radicals having from one to four carbon atoms in a straight or branched chain configuration, including, but not limited to, methyl, ethyl, n-propyl (also referred to as propyl or propanyl), isopropyl, n-butyl (also referred to as butyl or butanyl), isobutyl, sec-butyl, tert-butyl and the like. A C₁₋₄ alkyl radical is optionally substituted with substituent species as described herein where allowed by available valences.

As used herein, the term “C₂₋₄alkenyl” generally refers to partially unsaturated hydrocarbon radicals having from two to four carbon atoms in a straight or branched chain configuration and one or more carbon-carbon double bonds therein, including, but not limited to, ethenyl (also referred to as vinyl), allyl, propenyl and the like. A C₂₋₄alkenyl radical is optionally substituted with substituent species as described herein where allowed by available valences.

As used herein, the term “C₂₋₈alkynyl” generally refers to partially unsaturated hydrocarbon radicals having from two to eight carbon atoms in a straight or branched chain configuration and one or more carbon-carbon triple bonds therein, including, but not limited to, ethynyl, propynyl, butynyl and the like. In certain aspects, C₂₋₈alkynyl includes, but is not limited to, C₂₋₆alkynyl, C₂₋₄alkynyl and the like. A C₂₋₈alkynyl radical is optionally substituted with substituent species as described herein where allowed by available valences.

As used herein, the term “C₁₋₄alkoxy” generally refers to saturated hydrocarbon radicals having from one to four carbon atoms in a straight or branched chain configuration of the Formulae: —O—C₁₋₄alkyl, including, but not limited to, methoxy, ethoxy, n-propoxy, isopropoxy, n-butoxy, isobutoxy, sec-butoxy, tert-butoxy and the like. A C₁₋₄alkoxy radical is optionally substituted with substituent species as described herein where allowed by available valences.

As used herein, the term “C₃₋₁₀cycloalkyl” generally refers to a saturated or partially unsaturated monocyclic, bicyclic or polycyclic hydrocarbon radical, including, but not limited to, cyclopropyl, cyclobutyl, cyclopentyl, cyclohexyl, cyclohexenyl, cycloheptyl, and the like. A C₃₋₁₀cycloalkyl radical is optionally substituted with substituent species as described herein where allowed by available valences.

As used herein, the term “aryl” generally refers to a monocyclic, bicyclic or polycyclic aromatic carbon atom ring structure radical, including, but not limited to, phenyl, naphthyl, anthracenyl, fluorenyl, azulenyl, phenanthrenyl and the like. An aryl radical is optionally substituted with substituent species as described herein where allowed by available valences.

As used herein, the term “heteroaryl” generally refers to a monocyclic, bicyclic or polycyclic aromatic carbon atom ring structure radical in which one or more carbon atom ring members have been replaced, where allowed by structural stability, with one or more heteroatoms, such as an O, S or N atom, including, but not limited to, furanyl, thienyl, pyrrolyl, pyrazolyl, imidazolyl, isoxazolyl, isothiazolyl, oxazolyl, 1,3-thiazolyl, triazolyl, oxadiazolyl, thiadiazolyl, tetrazolyl, pyridinyl, pyrimidinyl, pyrazinyl, pyridazinyl, triazinyl, indolyl, indazolyl, indolizinyl, isoindolyl, benzofuranyl, benzothienyl, benzoimidazolyl, 1,3-benzothiazolyl, 1,3-benzoxazolyl, purinyl, quinolinyl, isoquinolinyl, quinazolinyl, quinoxalinyl, 1,3-diazinyl, 1,2-diazinyl, 1,2-diazolyl, 1,4-diazanaphthalenyl, acridinyl, furo[3,2-b]pyridinyl, furo[3,2-c]pyridinyl, furo[2,3-c]pyridinyl, 6H-thieno[2,3-b]pyrrolyl, thieno[3,2-c]pyridinyl, thieno[2,3-d]pyrimidinyl, 1H-pyrrolo[2,3-b]pyridinyl, 1H-pyrrolo[2,3-c]pyridinyl, 1H-pyrrolo[3,2-b]pyridinyl, pyrrolo[1,2-a]pyrazinyl, pyrrolo[1,2-b]pyridazinyl, pyrazolo[1,5-a]pyridinyl, pyrazolo[1,5-a]pyrazinyl, imidazo[1,2-a]pyridinyl, 3H-imidazo[4,5-b]pyridinyl, imidazo[1,2-a]pyrimidinyl, imidazo[1,2-c]pyrimidinyl, imidazo[1,2-b]pyridazinyl, imidazo[1,2-a]pyrazinyl, imidazo[2,1-b][1,3]thiazolyl, imidazo[2,1-b][1,3,4]thiadiazolyl, [1,2,4]triazolo[1,5-a]pyridinyl, [1,2,4]triazolo[4,3-a]pyridinyl and the like. A heteroaryl radical is optionally substituted on a carbon or nitrogen atom ring member with substituent species as described herein where allowed by available valences.

In certain aspects, the nomenclature for a heteroaryl radical may differ, such as in non-limiting examples where furanyl may also be referred to as furyl, thienyl may also be referred to as thiophenyl, pyridinyl may also be referred to as pyridyl, benzothienyl may also be referred to as benzothiophenyl and 1,3-benzoxazolyl may also be referred to as 1,3-benzooxazolyl.

In certain other aspects, the term for a heteroaryl radical may also include other regioisomers, such as in non-limiting examples where the term pyrrolyl may also include 2H-pyrrolyl, 3H-pyrrolyl and the like, the term pyrazolyl may also include 1H-pyrazolyl and the like, the term imidazolyl may also include 1H-imidazolyl and the like, the term triazolyl may also include 1H-1,2,3-triazolyl and the like, the term oxadiazolyl may also include 1,2,4-oxadiazolyl, 1,3,4-oxadiazolyl and the like, the term tetrazolyl may also include 1H-tetrazolyl, 2H-tetrazolyl and the like, the term indolyl may also include 1H-indolyl and the like, the term indazolyl may also include 1H-indazolyl, 2H-indazolyl and the like, the term benzoimidazolyl may also include 1H-benzoimidazolyl and the term purinyl may also include 9H-purinyl and the like.

As used herein, the term “heterocyclyl” generally refers to a saturated or partially unsaturated monocyclic, bicyclic or polycyclic carbon atom ring structure radical in which one or more carbon atom ring members have been replaced, where allowed by structural stability, with a heteroatom, such as an O, S or N atom, including, but not limited to, oxiranyl, oxetanyl, azetidinyl, tetrahydrofuranyl, pyrrolinyl, pyrrolidinyl, pyrazolinyl, pyrazolidinyl, imidazolinyl, imidazolidinyl, isoxazolinyl, isoxazolidinyl, isothiazolinyl, isothiazolidinyl, oxazolinyl, oxazolidinyl, thiazolinyl, thiazolidinyl, triazolinyl, triazolidinyl, oxadiazolinyl, oxadiazolidinyl, thiadiazolinyl, thiadiazolidinyl, tetrazolinyl, tetrazolidinyl, pyranyl, dihydro-2H-pyranyl, thiopyranyl, 1,3-dioxanyl, 1,2,5,6-tetrahydropyridinyl, 1,2,3,6-tetrahydropyridinyl, piperidinyl, piperazinyl, morpholinyl, thiomorpholinyl, 1,4-diazepanyl, 1,3-benzodioxolyl, 1,4-benzodioxanyl, 2,3-dihydro-1,4-benzodioxinyl, hexahydropyrrolo[3,4-b]pyrrol-(1H)-yl, (3aS,6aS)-hexahydropyrrolo[3,4-b]pyrrol-(1H)-yl, (3aR,6aR)-hexahydropyrrolo[3,4-b]pyrrol-(1H)-yl, hexahydropyrrolo[3,4-b]pyrrol-(2H)-yl, (3aS,6aS)-hexahydropyrrolo[3,4-b]pyrrol-(2H)-yl, (3aR,6aR)-hexahydropyrrolo[3,4-b]pyrrol-(2H)-yl, hexahydropyrrolo[3,4-c]pyrrol-(1H)-yl, (3aR,6aS)-hexahydropyrrolo[3,4-c]pyrrol-(1H)-yl, (3aR,6aR)-hexahydropyrrolo[3,4-c]pyrrol-(1H)-yl, octahydro-5H-pyrrolo[3,2-c]pyridinyl, octahydro-6H-pyrrolo[3,4-b]pyridinyl, (4aR,7aR)-octahydro-6H-pyrrolo[3,4-b]pyridinyl, (4aS,7aS)-octahydro-6H-pyrrolo[3,4-b]pyridinyl, hexahydropyrrolo[1,2-a]pyrazin-(1H)-yl, (7R,8aS)-hexahydropyrrolo[1,2-a]pyrazin-(1H)-yl, (8aS)-hexahydropyrrolo[1,2-a]pyrazin-(1H)-yl, (8aR)-hexahydropyrrolo[1,2-a]pyrazin-(1H)-yl, (8aS)-octahydropyrrolo[1,2-a]pyrazin-(1H)-yl, (8aR)-octahydropyrrolo[1,2-a]pyrazin-(1H)-yl, hexahydropyrrolo[1,2-a]pyrazin-(2H)-one, octahydro-2H-pyrido[1,2-a]pyrazinyl, 3-azabicyclo[3.1.0]hexyl, (1R,5S)-3-azabicyclo[3.1.0]hexyl, 8-azabicyclo[3.2.1]octyl, (1R,5S)-8-azabicyclo[3.2.1]octyl, 8-azabicyclo[3.2.1]oct-2-enyl, (1R,5S)-8-azabicyclo[3.2.1]oct-2-enyl, 9-azabicyclo[3.3.1]nonyl, (1R,5S)-9-azabicyclo[3.3.1]nonyl, 2,5-diazabicyclo[2.2.1]heptyl, (1S,4S)-2,5-diazabicyclo[2.2.1]heptyl, 2,5-diazabicyclo[2.2.2]octyl, 3,8-diazabicyclo[3.2.1]octyl, (1R,5S)-3,8-diazabicyclo[3.2.1]octyl, 1,4-diazabicyclo[3.2.2]nonyl, azaspiro[3.3]heptyl, 2,6-diazaspiro[3.3]heptyl, 2,6-diazaspiro[3.4]octyl, 2,7-diazaspiro[3.5]nonyl, 5,8-diazaspiro[3.5]nonyl, 2,7-diazaspiro[4.4]nonyl, 6,9-diazaspiro[4.5]decyl, 7-azadispiro[5.1.58.36]hexadecanyl and the like. A heterocyclyl radical is optionally substituted on a carbon or nitrogen atom ring member with substituent species as described herein where allowed by available valences.

In certain aspects, the nomenclature for a heterocyclyl radical may differ, such as in non-limiting examples where 1,3-benzodioxolyl may also be referred to as benzo[d][1,3]dioxolyl and 2,3-dihydro-1,4-benzodioxinyl may also be referred to as 2,3-dihydrobenzo[b][1,4]dioxinyl.

As used herein, the term “deutero-C₁₋₄alkyl,” refers to a radical of the Formulae: —C₁₋₄alkyl-deutero, wherein C₁₋₄alkyl is partially or completely substituted with one or more deuterium atoms where allowed by available valences.

For the purposes of this description, where one or more substituent variables for a compound of Formulae I-XI or a form thereof encompass functionalities incorporated into a compound of Formulae I-XI, each functionality appearing at any location within the disclosed compound may be independently selected, and as appropriate, independently and/or optionally substituted.

As used herein, the terms “independently selected,” or “each selected” refer to functional variables in a substituent list that may occur more than once on the structure of Formulae I-XI, the pattern of substitution at each occurrence is independent of the pattern at any other occurrence. Further, the use of a generic substituent variable on any Formulae or structure for a compound described herein is understood to include the replacement of the generic substituent with species substituents that are included within the particular genus, e.g., aryl may be replaced with phenyl or naphthalenyl and the like, and that the resulting compound is to be included within the scope of the compounds described herein.

As used herein, the terms “each instance of” or “in each instance, when present,” when used preceding a phrase such as “ . . . C₃₋₁₄cycloalkyl, C₃₋₁₄cycloalkyl-C₁₋₄alkyl, aryl, aryl-C₁₋₄alkyl, heteroaryl, heteroaryl-C₁₋₄alkyl, heterocyclyl and heterocyclyl-C₁₋₄alkyl,” are intended to refer to the C₃₋₁₄cycloalkyl, aryl, heteroaryl and heterocyclyl ring systems when each are present either alone or as a substituent.

As used herein, the term “optionally substituted” means optional substitution with the specified substituent variables, groups, radicals or moieties.

### Compound Forms

As used herein, the term “form” means a compound of Formulae I-XI having a form selected from the group consisting of a free acid, free base, prodrug, salt, hydrate, solvate, clathrate, isotopologue, racemate, enantiomer, diastereomer, stereoisomer, polymorph and tautomer form thereof.

In certain aspects described herein, the form of the compound of Formulae I-XI is a free acid, free base or salt thereof.

In certain aspects described herein, the form of the compound of Formulae I-XI is a salt thereof.

In certain aspects described herein, the form of the compound of Formulae I-XI is an isotopologue thereof.

In certain aspects described herein, the form of the compound of Formulae I-XI is a stereoisomer, racemate, enantiomer or diastereomer thereof.

In certain aspects described herein, the form of the compound of Formulae I-XI is a tautomer thereof.

In certain aspects described herein, the form of the compound of Formulae I-XI is a pharmaceutically acceptable form.

In certain aspects described herein, the compound of Formulae I-XI or a form thereof is isolated for use.

As used herein, the term “isolated” means the physical state of a compound of Formulae I-XI or a form thereof after being isolated and/or purified from a synthetic process (e.g., from a reaction mixture) or natural source or combination thereof according to an isolation or purification process or processes described herein or which are well known to the skilled artisan (e.g., chromatography, recrystallization and the like) in sufficient purity to be characterized by standard analytical techniques described herein or well known to the skilled artisan.

As used herein, the term “protected” means that a functional group in a compound of Formulae I-XI or a form thereof is in a form modified to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York. Such functional groups include hydroxy, phenol, amino and carboxylic acid. Suitable protecting groups for hydroxy or phenol include trialkylsilyl or diarylalkylsilyl (e.g., t-butyldimethylsilyl, t-butyldiphenylsilyl or trimethylsilyl), tetrahydropyranyl, benzyl, substituted benzyl, methyl, methoxymethanol, and the like. Suitable protecting groups for amino, amidino and guanidino include t-butoxycarbonyl, benzyloxycarbonyl, and the like. Suitable protecting groups for carboxylic acid include alkyl, aryl or arylalkyl esters. In certain instances, the protecting group may also be a polymer resin, such as a Wang resin or a 2-chlorotrityl-chloride resin. Protecting groups may be added or removed in accordance with standard techniques, which are well-known to those skilled in the art and as described herein. It will also be appreciated by those skilled in the art, although such protected derivatives of compounds described herein may not possess pharmacological activity as such, they may be administered to a subject and thereafter metabolized in the body to form compounds described herein which are pharmacologically active. Such derivatives may therefore be described as “prodrugs”. All prodrugs of compounds described herein are included within the scope of the use described herein.

As used herein, the term “prodrug” means a form of an instant compound (e.g., a drug precursor) that is transformed in vivo to yield an active compound of Formulae I-XI or a form thereof. The transformation may occur by various mechanisms (e.g., by metabolic and/or nonmetabolic chemical processes), such as, for example, by hydrolysis and/or metabolism in blood, liver and/or other organs and tissues. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

In one example, when a compound of Formulae I-XI or a form thereof contains a carboxylic acid functional group, a prodrug can comprise an ester formed by the replacement of the hydrogen atom of the acid group with a functional group such as alkyl and the like. In another example, when a compound of Formulae I-XI or a form thereof contains a hydroxyl functional group, a prodrug form can be prepared by replacing the hydrogen atom of the hydroxyl with another functional group such as alkyl, alkylcarbonyl or a phosphonate ester and the like. In another example, when a compound of Formulae I-XI or a form thereof contains an amine functional group, a prodrug form can be prepared by replacing one or more amine hydrogen atoms with a functional group such as alkyl or substituted carbonyl. Pharmaceutically acceptable prodrugs of compounds of Formulae I-XI or a form thereof include those compounds substituted with one or more of the following groups: carboxylic acid esters, sulfonate esters, amino acid esters, phosphonate esters and mono-, di- or triphosphate esters or alkyl substituents, where appropriate. As described herein, it is understood by a person of ordinary skill in the art that one or more of such substituents may be used to provide a compound of Formulae I-XI or a form thereof as a prodrug.

One or more compounds described herein may exist in unsolvated as well as solvated forms with pharmaceutically acceptable solvents such as water, ethanol, and the like, and the description herein is intended to embrace both solvated and unsolvated forms.

As used herein, the term “solvate” means a physical association of a compound described herein with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. As used herein, “solvate” encompasses both solution-phase and isolatable solvates. Non-limiting examples of suitable solvates include ethanolates, methanolates, and the like.

The compounds of Formulae I-XI can form salts, which are intended to be included within the scope of this description. Reference to a compound of Formulae I-XI or a form thereof herein is understood to include reference to salt forms thereof, unless otherwise indicated. The term “salt(s)”, as employed herein, denotes acidic salts formed with inorganic and/or organic acids, as well as basic salts formed with inorganic and/or organic bases. In addition, when a compound of Formulae I-XI or a form thereof contains both a basic moiety, such as, without limitation an amine moiety, and an acidic moiety, such as, but not limited to a carboxylic acid, zwitterions (“inner salts”) may be formed and are included within the term “salt(s)” as used herein.

The term “pharmaceutically acceptable salt(s)”, as used herein, means those salts of compounds described herein that are safe and effective (i.e., non-toxic, physiologically acceptable) for use in mammals and that possess biological activity, although other salts are also useful. Salts of the compounds of the Formulae I-XI may be formed, for example, by reacting a compound of Formulae I-XI or a form thereof with an amount of acid or base, such as an equivalent amount, in a medium such as one in which the salt precipitates or in an aqueous medium followed by lyophilization.

Pharmaceutically acceptable salts include one or more salts of acidic or basic groups present in compounds described herein. Particular aspects of acid addition salts include, and are not limited to, acetate, ascorbate, benzoate, benzenesulfonate, bisulfate, bitartrate, borate, bromide, butyrate, chloride, citrate, camphorate, camphorsulfonate, ethanesulfonate, formate, fumarate, gentisinate, gluconate, glucaronate, glutamate, iodide, isonicotinate, lactate, maleate, methanesulfonate, naphthalenesulfonate, nitrate, oxalate, pamoate, pantothenate, phosphate, propionate, saccharate, salicylate, succinate, sulfate, tartrate, thiocyanate, toluenesulfonate (also known as tosylate), trifluoroacetate salts and the like. Certain particular aspects of acid addition salts include chloride or dichloride.

Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33, 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Suitable basic salts include, but are not limited to, aluminum, ammonium, calcium, lithium, magnesium, potassium, sodium and zinc salts.

All such acid salts and base salts are intended to be included within the scope of pharmaceutically acceptable salts as described herein. In addition, all such acid and base salts are considered equivalent to the free forms of the corresponding compounds for purposes of this description.

Compounds of Formulae I-XI and forms thereof, may further exist in a tautomeric form. All such tautomeric forms are contemplated and intended to be included within the scope of the compounds of Formulae I-XI or a form thereof as described herein.

The compounds of Formulae I-XI or a form thereof may contain asymmetric or chiral centers, and, therefore, exist in different stereoisomeric forms. The present description is intended to include all stereoisomeric forms of the compounds of Formulae I-XI as well as mixtures thereof, including racemic mixtures.

The compounds described herein may include one or more chiral centers, and as such may exist as racemic mixtures (R S) or as substantially pure enantiomers and diastereomers. The compounds may also exist as substantially pure (R) or (S) enantiomers (when one chiral center is present). In one particular aspect, the compounds described herein are (S) isomers and may exist as enantiomerically pure compositions substantially comprising only the (S) isomer. In another particular aspect, the compounds described herein are (R) isomers and may exist as enantiomerically pure compositions substantially comprising only the (R) isomer. As one of skill in the art will recognize, when more than one chiral center is present, the compounds described herein may also exist as a (R,R), (R,S), (S,R) or (S,S) isomer, as defined by IUPAC Nomenclature Recommendations.

As used herein, the term “substantially pure” refers to compounds consisting substantially of a single isomer in an amount greater than or equal to 90%, in an amount greater than or equal to 92%, in an amount greater than or equal to 95%, in an amount greater than or equal to 98%, in an amount greater than or equal to 99%, or in an amount equal to 100% of the single isomer.

In one aspect of the description, a compound of Formulae I-XI or a form thereof is a substantially pure (S) enantiomer form present in an amount greater than or equal to 90%, in an amount greater than or equal to 92%, in an amount greater than or equal to 95%, in an amount greater than or equal to 98%, in an amount greater than or equal to 99%, or in an amount equal to 100%.

In one aspect of the description, a compound of Formulae I-XI or a form thereof is a substantially pure (R) enantiomer form present in an amount greater than or equal to 90%, in an amount greater than or equal to 92%, in an amount greater than or equal to 95%, in an amount greater than or equal to 98%, in an amount greater than or equal to 99%, or in an amount equal to 100%.

As used herein, a “racemate” is any mixture of isometric forms that are not “enantiomerically pure”, including mixtures such as, without limitation, in a ratio of about 50/50, about 60/40, about 70/30, or about 80/20.

In addition, the present description embraces all geometric and positional isomers. For example, if a compound of Formulae I-XI or a form thereof incorporates a double bond or a fused ring, both the cis- and trans-forms, as well as mixtures, are embraced within the scope of the description. Diastereomeric mixtures can be separated into their individual diastereomers on the basis of their physical chemical differences by methods well known to those skilled in the art, such as, for example, by chromatography and/or fractional crystallization. Enantiomers can be separated by use of chiral HPLC column or other chromatographic methods known to those skilled in the art. Enantiomers can also be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formulae I-XI may be atropisomers (e.g., substituted biaryls) and are considered as part of this description.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this description, as are positional isomers (such as, for example, 4-pyridyl and 3-pyridyl). Individual stereoisomers of the compounds described herein may, for example, be substantially free of other isomers, or may be present in a racemic mixture, as described supra.

The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or isotopologues of the instant compounds.

The term “isotopologue” refers to isotopically-enriched compounds described herein which are identical to those recited herein, but for the fact that one or more atoms are replaced by an atom having an atomic mass or mass number different from the atomic mass or mass number usually found in nature. Examples of isotopes that can be incorporated into compounds described herein include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, ³⁵Cl and ³⁶Cl, respectively, each of which are also within the scope of this description.

Certain isotopically-enriched compounds described herein (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Polymorphic crystalline and amorphous forms of the compounds of Formulae I-XI and of the salts, solvates, hydrates, esters and prodrugs of the compounds of Formulae I-XI are further intended to be included in the present description.

### Compound Uses

In accordance with the intended scope of the present description, aspects of the present description include compounds that have been identified and have been demonstrated to be useful in selectively preventing, treating or ameliorating any disease that is mediated by NLRP3 and have been provided for use for preventing, treating or ameliorating any disease that is mediated by NLRP3.

An aspect of the present description includes a method for preventing, treating or ameliorating any disease that is mediated by NLRP3 in a subject in need thereof comprising, administering to the subject an effective amount of a compound of Formulae I-XI or a form thereof.

An aspect of the present description includes a method for treating or ameliorating any disease that is mediated by NLRP3 in a subject in need thereof comprising, administering to the subject an effective amount of a compound of Formulae I-XI or a form thereof.

An aspect of the present description includes a method for preventing any disease that is mediated by NLRP3 in a subject in need thereof comprising, administering to the subject an effective amount of a compound of Formulae I-XI or a form thereof.

An aspect of the present description includes a method for treating any disease that is mediated by NLRP3 in a subject in need thereof comprising, administering to the subject an effective amount of a compound of Formulae I-XI or a form thereof.

An aspect of the present description includes a method for ameliorating any disease that is mediated by NLRP3 in a subject in need thereof comprising, administering to the subject an effective amount of a compound of Formulae I-XI or a form thereof.

Another aspect of the present description includes a method for treating or ameliorating any disease that is mediated by NLRP3 in a subject in need thereof comprising, administering to the subject an effective amount of a compound salt of Formulae I-XI or a form thereof.

An aspect of the present description includes a method for use of a compound of Formulae I-XI or a form or composition thereof for treating or ameliorating any disease that is mediated by NLRP3 in a subject in need thereof comprising, administering to the subject an effective amount of the compound of Formulae I-XI or a form or composition thereof.

Another aspect of the present description includes a method for use of a compound salt of Formulae I-XI or a form or composition thereof for treating or ameliorating any disease that is mediated by NLRP3 in a subject in need thereof comprising, administering to the subject an effective amount of the compound salt of Formulae I-XI or a form thereof.

An aspect of the present description includes a use for a compound of Formulae I-XI or a form thereof for treating or ameliorating any disease that is mediated by NLRP3 in a subject in need thereof comprising, administering to the subject an effective amount of the compound of Formulae I-XI or a form thereof.

Another aspect of the present description includes a use for a compound salt of Formulae I-XI or a form thereof for treating or ameliorating any disease that is mediated by NLRP3 in a subject in need thereof comprising, administering to the subject an effective amount of the compound salt of Formulae I-XI or a form thereof.

An aspect of the present description includes a use for a compound of Formulae I-XI or a form thereof in the manufacture of a medicament for treating or ameliorating any disease that is mediated by NLRP3 in a subject in need thereof comprising, administering to the subject an effective amount of the medicament.

Another aspect of the present description includes a use for a compound salt of Formulae I-XI or a form thereof in the manufacture of a medicament for treating or ameliorating any disease that is mediated by NLRP3 in a subject in need thereof comprising, administering to the subject an effective amount of the medicament.

An aspect of the present description includes in vitro or in vivo use of the compound of Formulae I-XI or a form thereof having activity toward any disease that is mediated by NLRP3.

An aspect of the present description includes a use of the compound of Formulae I-XI or a form thereof in a combination therapy to provide additive or synergistic activity, thus enabling the development of a combination product for treating or ameliorating any disease that is mediated by NLRP3.

Another aspect of the present description includes a combination therapy comprising compounds described herein in combination with one or more known drugs or one or more known therapies may be used to treat any disease that is mediated by NLRP3 regardless of whether any disease that is mediated by NLRP3 is responsive to the known drug.

An aspect of the present description includes a use for a compound of Formulae I-XI or a form thereof in a combination product with one or more therapeutic agents for treating or ameliorating any disease that is mediated by NLRP3 in a subject in need thereof comprising, administering to the subject an effective amount of the compound of Formulae I-XI or a form thereof in combination with an effective amount of the one or more agents.

Another aspect of the present description includes a use for a compound salt of Formulae I-XI or a form thereof in a combination product with one or more therapeutic agents for treating or ameliorating any disease that is mediated by NLRP3 in a subject in need thereof comprising, administering to the subject an effective amount of the compound salt of Formulae I-XI or a form thereof in combination with an effective amount of the one or more agents. In an aspect of a use or method provided herein, compounds of Formulae I-XI or a form thereof used in combination with one or more additional agents can be administered to a subject or contacted with a subject or patient cell(s) prior to, concurrently with, or subsequent to administering to the subject or patient or contacting the cell with an additional agent(s). A compound(s) of Formulae I-XI or a form thereof and an additional agent(s) can be administered to a subject or contacted with a cell in single composition or different compositions. In a specific aspect, a compound(s) of Formulae I-XI or a form thereof is used in combination with gene therapy to inhibit NLRP3 expression (using, e.g., viral delivery vectors) or the administration of another small molecule Nlrp3 inhibitor. In another specific aspect, a compound(s) of Formulae I-XI or a form thereof are used in combination with cell replacement using differentiated non-mutant NLRP3 stem cells. In another specific aspect, a compound(s) of Formulae I-XI or a form thereof are used in combination with cell replacement using differentiated NLRP3 stem cells.

In one aspect, provided herein is the use of compounds of Formulae I-XI or a form thereof in combination with supportive standard of care therapies, including palliative care.

An aspect of the present description includes a use for a compound of Formulae I-XI or a form thereof in the preparation of a kit for treating or ameliorating any disease that is mediated by NLRP3 in a subject in need thereof comprising, the compound of Formulae I-XI or a form thereof and instructions for administering an effective amount of the compound of Formulae I-XI or a form thereof.

An aspect of the present description includes a use for a compound of Formulae I-XI or a form thereof in the preparation of a kit for treating or ameliorating any disease that is mediated by NLRP3 in a subject in need thereof comprising, the compound of Formulae I-XI or a form thereof and instructions for administering an effective amount of the compound of Formulae I-XI or a form thereof, and optionally, for administering to the subject an effective amount of the compound of Formulae I-XI or a form thereof in a combination product with an effective amount of one or more therapeutic agents.

An aspect of the present description includes a use for a compound of Formulae I-XI or a form thereof in the preparation of a kit for treating or ameliorating any disease that is mediated by NLRP3 in a subject in need thereof comprising, the compound of Formulae I-XI or a form thereof and instructions for administering an effective amount of the compound of Formulae I-XI or a form thereof, and optionally, for administering to the subject an effective amount of the compound of Formulae I-XI or a form thereof in a combination product with an effective amount of the one or more therapeutic agents; and optionally, for administering to the subject an effective amount of the compound of Formulae I-XI or a form thereof in a combination product with an effective amount of the one or more therapeutic agents in a combination therapy with a standard of care supportive therapy, wherein the standard of care supportive therapy is palliative care.

In one respect, for each of such aspects, the subject is treatment naive. In another respect, for each of such aspects, the subject is not treatment naive.

As used herein, the term “preventing” refers to keeping a disease, disorder or condition from occurring in a subject that may be predisposed to the disease, disorder and/or condition but has not yet been diagnosed as having the disease, disorder and/or condition.

As used herein, the term “treating” refers to inhibiting the progression of a disease, disorder or condition in a subject already exhibiting the symptoms of the disease, disorder and/or condition, i.e., arresting the development of a disease, disorder and/or condition that has already affected the subject.

As used herein, the term “ameliorating” refers to relieving the symptoms of a disease, disorder or condition in a subject already exhibiting the symptoms of the disease, disorder and/or condition, i.e., causing regression of the disease, disorder and/or condition that has already affected the subject.

As used herein, the term “subject” refers to an animal or any living organism having sensation and the power of voluntary movement, and which requires oxygen and organic food.

Nonlimiting examples include members of the human, primate, equine, porcine, bovine, murine, rattus, canine and feline specie. In certain aspects, the subject is a mammal or a warm-blooded vertebrate animal. In other aspects, the subject is a human. As used herein, the term “patient” may be used interchangeably with “subject” and “human”.

As used herein, the terms “effective amount” or “therapeutically effective amount” mean an amount of compound of Formulae I-XI or a form, composition or medicament thereof that achieves a target plasma concentration that is effective in treating or ameliorating any disease that is mediated by NLRP3 as described herein and thus producing the desired therapeutic, ameliorative, inhibitory or preventative effect in a subject in need thereof. In one aspect, the effective amount may be the amount required to treat any disease that is mediated by NLRP3 in a subject or patient, more specifically, in a human.

In another aspect, the concentration-biological effect relationships observed with regard to a compound of Formulae I-XI or a form thereof indicate a target plasma concentration ranging from approximately 0.001 μg/mL to approximately 50 μg/mL, from approximately 0.01 μg/mL to approximately 20 μg/mL, from approximately 0.05 μg/mL to approximately 10 μg/mL, or from approximately 0.1 μg/mL to approximately 5 μg/mL. To achieve such plasma concentrations, the compounds described herein may be administered at doses that vary, such as, for example, without limitation, from 1.0 ng to 10,000 mg.

In one aspect, the dose administered to achieve an effective target plasma concentration may be administered based upon subject or patient specific factors, wherein the doses administered on a weight basis may be in the range of from about 0.001 mg/kg/day to about 3500 mg/kg/day, or about 0.001 mg/kg/day to about 3000 mg/kg/day, or about 0.001 mg/kg/day to about 2500 mg/kg/day, or about 0.001 mg/kg/day to about 2000 mg/kg/day, or about 0.001 mg/kg/day to about 1500 mg/kg/day, or about 0.001 mg/kg/day to about 1000 mg/kg/day, or about 0.001 mg/kg/day to about 500 mg/kg/day, or about 0.001 mg/kg/day to about 250 mg/kg/day, or about 0.001 mg/kg/day to about 200 mg/kg/day, or about 0.001 mg/kg/day to about 150 mg/kg/day, or about 0.001 mg/kg/day to about 100 mg/kg/day, or about 0.001 mg/kg/day to about 75 mg/kg/day, or about 0.001 mg/kg/day to about 50 mg/kg/day, or about 0.001 mg/kg/day to about 25 mg/kg/day, or about 0.001 mg/kg/day to about 10 mg/kg/day, or about 0.001 mg/kg/day to about 5 mg/kg/day, or about 0.001 mg/kg/day to about 1 mg/kg/day, or about 0.001 mg/kg/day to about 0.5 mg/kg/day, or about 0.001 mg/kg/day to about 0.1 mg/kg/day, or from about 0.01 mg/kg/day to about 3500 mg/kg/day, or about 0.01 mg/kg/day to about 3000 mg/kg/day, or about 0.01 mg/kg/day to about 2500 mg/kg/day, or about 0.01 mg/kg/day to about 2000 mg/kg/day, or about 0.01 mg/kg/day to about 1500 mg/kg/day, or about 0.01 mg/kg/day to about 1000 mg/kg/day, or about 0.01 mg/kg/day to about 500 mg/kg/day, or about 0.01 mg/kg/day to about 250 mg/kg/day, or about 0.01 mg/kg/day to about 200 mg/kg/day, or about 0.01 mg/kg/day to about 150 mg/kg/day, or about 0.01 mg/kg/day to about 100 mg/kg/day, or about 0.01 mg/kg/day to about 75 mg/kg/day, or about 0.01 mg/kg/day to about 50 mg/kg/day, or about 0.01 mg/kg/day to about 25 mg/kg/day, or about 0.01 mg/kg/day to about 10 mg/kg/day, or about 0.01 mg/kg/day to about 5 mg/kg/day, or about 0.01 mg/kg/day to about 1 mg/kg/day, or about 0.01 mg/kg/day to about 0.5 mg/kg/day, or about 0.01 mg/kg/day to about 0.1 mg/kg/day, or from about 0.1 mg/kg/day to about 3500 mg/kg/day, or about 0.1 mg/kg/day to about 3000 mg/kg/day, or about 0.1 mg/kg/day to about 2500 mg/kg/day, or about 0.1 mg/kg/day to about 2000 mg/kg/day, or about 0.1 mg/kg/day to about 1500 mg/kg/day, or about 0.1 mg/kg/day to about 1000 mg/kg/day, or about 0.1 mg/kg/day to about 500 mg/kg/day, or about 0.1 mg/kg/day to about 250 mg/kg/day, or about 0.1 mg/kg/day to about 200 mg/kg/day, or about 0.1 mg/kg/day to about 150 mg/kg/day, or about 0.1 mg/kg/day to about 100 mg/kg/day, or about 0.1 mg/kg/day to about 75 mg/kg/day, or about 0.1 mg/kg/day to about 50 mg/kg/day, or about 0.1 mg/kg/day to about 25 mg/kg/day, or about 0.1 mg/kg/day to about 10 mg/kg/day, or about 0.1 mg/kg/day to about 5 mg/kg/day, or about 0.1 mg/kg/day to about 1 mg/kg/day, or about 0.1 mg/kg/day to about 0.5 mg/kg/day.

Effective amounts for a given subject may be determined by routine experimentation that is within the skill and judgment of a clinician or a practitioner skilled in the art in light of factors related to the subject. Dosage and administration may be adjusted to provide sufficient levels of the active agent(s) or to maintain the desired effect. Factors which may be taken into account include genetic screening, severity of the disease state, status of disease progression, general health of the subject, ethnicity, age, weight, gender, diet, time of day and frequency of administration, drug combination(s), reaction sensitivities, experience with other therapies, and tolerance/response to therapy.

The dose administered to achieve an effective target plasma concentration may be orally administered once (once in approximately a 24 hour period; i.e., “q.d.”), twice (once in approximately a 12 hour period; i.e., “b.i.d.” or “q.12 h”), thrice (once in approximately an 8 hour period; i.e., “t.i.d.” or “q.8 h”), or four times (once in approximately a 6 hour period; i.e., “q.d.s.”, “q.i.d.” or “q.6 h”) daily.

In certain aspects, the dose administered to achieve an effective target plasma concentration may also be administered in a single, divided, or continuous dose for a patient or subject having a weight in a range of between about 40 to about 200 kg (which dose may be adjusted for patients or subjects above or below this range, particularly children under 40 kg). The typical adult subject is expected to have a median weight in a range of about 70 kg. Long-acting pharmaceutical compositions may be administered every 2, 3 or 4 days, once every week, or once every two weeks depending on half-life and clearance rate of the particular Formulation.

The compounds and compositions described herein may be administered to the subject via any drug delivery route known in the art. Nonlimiting examples include oral, ocular, rectal, buccal, topical, nasal, sublingual, transdermal, subcutaneous, intramuscular, intraveneous (bolus and infusion), intracerebral, and pulmonary routes of administration.

In another aspect, the dose administered may be adjusted based upon a dosage form described herein Formulated for delivery at about 0.02, 0.025, 0.03, 0.05, 0.06, 0.075, 0.08, 0.09, 0.10, 0.20, 0.25, 0.30, 0.50, 0.60, 0.75, 0.80, 0.90, 1.0, 1.10, 1.20, 1.25, 1.50, 1.75, 2.0, 3.0, 5.0, 10, 20, 30, 40, 50, 100, 150, 200, 250, 300, 400, 500, 1000, 1500, 2000, 2500, 3000 or 4000 mg/day.

For any compound, the effective amount can be estimated initially either in cell culture assays or in relevant animal models, such as a mouse, guinea pig, chimpanzee, marmoset or tamarin animal model. Relevant animal models may also be used to determine the appropriate concentration range and route of administration. Such information can then be used to determine useful doses and routes for administration in humans. Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is therapeutic index, and can be expressed as the ratio, LD₅₀/ED₅₀. In certain aspects, the effective amount is such that a large therapeutic index is achieved. In further particular aspects, the dosage is within a range of circulating concentrations that include an ED₅₀ with little or no toxicity. The dosage may vary within this range depending upon the dosage form employed, sensitivity of the patient, and the route of administration.

In one aspect, provided herein are methods for modulating the amount of NLRP3, comprising contacting a human cell with a compound of Formulae I-XI or a form thereof. In a specific aspect, provided herein are methods for modulating the amount of NLRP3, comprising contacting a human cell with a compound of Formulae I-XI or a form thereof that modulates the expression of NLRP3. The human cell can be contacted with a compound of Formulae I-XI or a form thereof in vitro, or in vivo, e.g., in a non-human animal or in a human. In a specific aspect, the human cell is from or in a human. In another specific aspect, the human cell is from or in a human with any disease that is modulated by NLRP3. In another specific aspect, the human cell is from or in a human with any disease that is modulated by NLRP3, resulting in a loss of NLRP #expression and/or function. In another aspect, the human cell is from a human with H any disease that is modulated by NLRP3. In another aspect, the human cell is in a human with any disease that is modulated by NLRP3. In one aspect, the compound is a form of a compound of Formulae I-XI.

In a specific aspect, provided herein is a method for enhancing the inhibition of mutant NLRP #transcribed from the NLRP3 gene, comprising contacting a human cell with a compound of Formulae I-XI or a form thereof. The human cell can be contacted with a compound of Formulae I-XI or a form thereof in vitro, or in vivo, e.g., in a non-human animal or in a human.

In a specific aspect, the human cell is from or in a human. In another specific aspect, the human cell is from or in a human with any disease that is modulated by NLRP3. In another specific aspect, the human cell is from or in a human with HD, caused by a CAG repeat in the NLRP3 gene, resulting in a loss of wild-type “normal” NLRP #expression and/or function. In another aspect, the human cell is from a human with any disease that is modulated by NLRP3. In another aspect, the human cell is in a human with any disease that is modulated by NLRP3. In one aspect, the compound is a form of the compound of Formulae I-XI.

In another aspect, provided herein is a method for modulating the inhibition of mutant NLRP3 transcribed from the NLRP3 gene, comprising administering to a non-human animal model for any disease that is modulated by NLRP3 a compound of Formulae I-XI or a form thereof. In a specific aspect, provided herein is a method for modulating the inhibition of mutant NLRP3 transcribed from the HNLRP3 gene, comprising administering to a non-human animal model for any disease that is modulated by NLRP3 a compound of Formulae I-XI or a form thereof. In a specific aspect, the compound is a form of the compound of Formulae I-XI.

In another aspect, provided herein is a method for decreasing the amount of mutant NLRP3, comprising contacting a human cell with a compound of Formulae I-XI or a form thereof. In a specific aspect, provided herein is a method for decreasing the amount of mutant NLRP3, comprising contacting a human cell with a compound of Formulae I-XI that inhibits the transcription of mutant NLRP3 from the NLRP3 gene. In another specific aspect, provided herein is a method for decreasing the amount of NLRP3, comprising contacting a human cell with a compound of Formulae I-XI that inhibits the expression of mutant NLRP3 transcribed from the NLRP3 gene. The human cell can be contacted with a compound of Formulae I-XI or a form thereof in vitro, or in vivo, e.g., in a non-human animal or in a human. In a specific aspect, the human cell is from or in a human. In another specific aspect, the human cell is from or in a human with any disease that is modulated by NLRP3. In another specific aspect, the human cell is from or in a human with any disease that is modulated by NLRP3. In another aspect, the human cell is from a human with any disease that is modulated by NLRP3. In another aspect, the human cell is in a human with any disease that is modulated by NLRP3. In one aspect, the compound is a form of the compound of Formulae I-XI.

In certain aspects, treating or ameliorating any disease that is modulated by NLRP3 with a compound of Formulae I-XI or a form thereof (alone or in combination with an additional agent) has a therapeutic effect and/or beneficial effect. In a specific aspect, treating any disease that is modulated by NLRP3 with a compound of Formulae I-XI or a form thereof (alone or in combination with an additional agent) results in one, two or more of the following effects: (i) reduces or ameliorates the severity of any disease that is modulated by NLRP3; (ii) delays onset of any disease that is modulated by NLRP3; (iii) inhibits the progression of any disease that is modulated by NLRP3; (iv) reduces hospitalization of a subject; (v) reduces hospitalization length for a subject; (vi) increases the survival of a subject; (vii) improves the quality of life for a subject; (viii) reduces the number of symptoms associated with any disease that is modulated by NLRP3; (ix) reduces or ameliorates the severity of a symptom(s) associated with any disease that is modulated by NLRP3; (x) reduces the duration of a symptom associated with any disease that is modulated by NLRP3; (xi) prevents the recurrence of a symptom associated with any disease that is modulated by NLRP3; (xii) inhibits the development or onset of a symptom of any disease that is modulated by NLRP3; and/or (xiii) inhibits of the progression of a symptom associated with any disease that is modulated by NLRP3.

### Metabolites

Another aspect included within the scope of the present description are the use of in vivo metabolic products of the compounds described herein. Such products may result, for example, from the oxidation, reduction, hydrolysis, amidation, esterification and the like of the administered compound, primarily due to enzymatic processes. Accordingly, the description includes the use of compounds produced by a process comprising contacting a compound described herein with a mammalian tissue or a mammal for a period of time sufficient to yield a metabolic product thereof.

Such products typically are identified by preparing a radio-labeled isotopologue (e.g., ¹⁴C or ³H) of a compound described herein, administering the radio-labeled compound in a detectable dose (e.g., greater than about 0.5 mg/kg) to a mammal such as a rat, mouse, guinea pig, dog, monkey or human, allowing sufficient time for metabolism to occur (typically about 30 seconds to about 30 hours), and identifying the metabolic conversion products from urine, bile, blood or other biological samples. The conversion products are easily isolated since they are “radiolabeled” by virtue of being isotopically-enriched (others are isolated by the use of antibodies capable of binding epitopes surviving in the metabolite). The metabolite structures are determined in conventional fashion, e.g., by MS or NMR analysis. In general, analysis of metabolites may be done in the same way as conventional drug metabolism studies well-known to those skilled in the art. The conversion products, so long as they are not otherwise found in vivo, are useful in diagnostic assays for therapeutic dosing of the compounds described herein even if they possess no biological activity of their own.

### Pharmaceutical Compositions

In accordance with the intended scope of the present description, aspects of the present description include compounds that have been identified and have been demonstrated to be useful in selectively preventing, treating or ameliorating any disease that is modulated by NLRP3 and have been provided for use as one or more pharmaceutical compositions for preventing, treating or ameliorating any disease that is modulated by NLRP3.

An aspect of the present description includes a use for a compound of Formulae I-XI or a form thereof in the preparation of a pharmaceutical composition for treating or ameliorating any disease that is modulated by NLRP3 in a subject in need thereof comprising, administering to the subject an effective amount of the compound of Formulae I-XI or a form thereof in admixture with one or more pharmaceutically acceptable excipients.

An aspect of the present description includes a use for a pharmaceutical composition of the compound of Formulae I-XI or a form thereof in the preparation of a kit for treating or ameliorating any disease that is modulated by NLRP3 in a subject in need thereof comprising, the pharmaceutical composition of the compound of Formulae I-XI or a form thereof and instructions for administering the pharmaceutical composition.

As used herein, the term “composition” means a product comprising the specified ingredients in the specified amounts, as well as any product which results, directly or indirectly, from combination of the specified ingredients in the specified amounts.

The pharmaceutical composition may be formulated to achieve a physiologically compatible pH, ranging from about pH 3 to about pH 11. In certain aspects, the pharmaceutical composition is formulated to achieve a pH of from about pH 3 to about pH 7. In other aspects, the pharmaceutical composition is formulated to achieve a pH of from about pH 5 to about pH 8. The term “pharmaceutically acceptable excipient” refers to an excipient for administration of a pharmaceutical agent, such as the compounds described herein. The term refers to any pharmaceutical excipient that may be administered without undue toxicity. Pharmaceutically acceptable excipients may be determined in part by the particular composition being administered, as well as by the particular mode of administration and/or dosage form. Nonlimiting examples of pharmaceutically acceptable excipients include carriers, solvents, stabilizers, adjuvants, diluents, etc. Accordingly, there exists a wide variety of suitable formulations of pharmaceutical compositions for the instant compounds described herein (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive antibodies. Other exemplary excipients include antioxidants such as ascorbic acid; chelating agents such as EDTA; carbohydrates such as dextrin, hydroxyalkylcellulose, hydroxyalkylmethylcellulose (e.g., hydroxypropylmethylcellulose, also known as HPMC), stearic acid; liquids such as oils, water, saline, glycerol and ethanol; wetting or emulsifying agents; pH buffering substances; and the like. Liposomes are also included within the definition of pharmaceutically acceptable excipients.

The pharmaceutical compositions described herein may be formulated in any form suitable for the intended use described herein. Suitable formulations for oral administration include solids, liquid solutions, emulsions and suspensions, while suitable inhalable formulations for pulmonary administration include liquids and powders. Alternative formulations include syrups, creams, ointments, tablets, and lyophilized solids which can be reconstituted with a physiologically compatible solvent prior to administration.

When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents, and preserving agents, in order to provide a palatable preparation.

Pharmaceutically acceptable excipients suitable for use in conjunction with tablets include, for example, inert diluents, such as celluloses, calcium or sodium carbonate, lactose, calcium or sodium phosphate; disintegrating agents, such as croscarmellose sodium, cross-linked povidone, maize starch, or alginic acid; binding agents, such as povidone, starch, gelatin or acacia; and lubricating agents, such as magnesium stearate, stearic acid, or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example celluloses, lactose, calcium phosphate, or kaolin, or as soft gelatin capsules wherein the active ingredient is mixed with non-aqueous or oil medium, such as glycerin, propylene glycol, polyethylene glycol, peanut oil, liquid paraffin, or olive oil.

In other aspects, pharmaceutical compositions described herein may be formulated as suspensions comprising a compound of Formulae I-XI, or a form thereof in admixture with one or more pharmaceutically acceptable excipients suitable for the manufacture of a suspension. In yet other aspects, pharmaceutical compositions described herein may be formulated as dispersible powders and granules suitable for preparation of a suspension by the addition of one or more excipients.

Excipients suitable for use in connection with suspensions include suspending agents, such as sodium carboxymethylcellulose, methylcellulose, hydroxypropyl methylcelluose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); and thickening agents, such as carbomer, beeswax, hard paraffin, or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

The pharmaceutical compositions described herein may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Additionally, the pharmaceutical compositions described herein may be in the form of a sterile injectable preparation, such as a sterile injectable aqueous emulsion or oleaginous suspension. Such emulsion or suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,2-propanediol. The sterile injectable preparation may also be prepared as a lyophilized powder. Among the acceptable vehicles and solvents that may be employed are water, Ringer's solution and isotonic sodium chloride solution. In addition, sterile fixed oils may be employed as a solvent or suspending medium. For this purpose any bland fixed oil may be employed including synthetic mono- or di-glycerides. In addition, fatty acids such as oleic acid may likewise be used in the preparation of injectables.

The compounds described herein may be substantially insoluble in water and sparingly soluble in most pharmaceutically acceptable protic solvents and vegetable oils, but generally soluble in medium-chain fatty acids (e.g., caprylic and capric acids) or triglycerides and in propylene glycol esters of medium-chain fatty acids. Thus, contemplated in the description are compounds which have been modified by substitutions or additions of chemical or biochemical moieties which make them more suitable for delivery (e.g., increase solubility, bioactivity, palatability, decrease adverse reactions, etc.), for example by esterification, glycosylation, PEGylation, etc.

In certain aspects, the compound described herein is formulated for oral administration in a lipid-based composition suitable for low solubility compounds. Lipid-based formulations can generally enhance the oral bioavailability of such compounds. As such, pharmaceutical compositions described herein may comprise a effective amount of a compound of Formulae I-XI or a form thereof, together with at least one pharmaceutically acceptable excipient selected from medium chain fatty acids or propylene glycol esters thereof (e.g., propylene glycol esters of edible fatty acids such as caprylic and capric fatty acids) and pharmaceutically acceptable surfactants, such as polysorbate 20 or 80 (also referred to as Tween® 20 or Tween® 80, respectively) or polyoxyl 40 hydrogenated castor oil.

In other aspects, the bioavailability of low solubility compounds may be enhanced using particle size optimization techniques including the preparation of nanoparticles or nanosuspensions using techniques known to those skilled in the art. The compound forms present in such preparations include amorphous, partially amorphous, partially crystalline or crystalline forms.

In alternative aspects, the pharmaceutical composition may further comprise one or more aqueous solubility enhancer(s), such as a cyclodextrin. Nonlimiting examples of cyclodextrin include hydroxypropyl, hydroxyethyl, glucosyl, maltosyl and maltotriosyl derivatives of α-, β-, and γ-cyclodextrin, and hydroxypropyl-β-cyclodextrin (HIPBC). In certain aspects, the pharmaceutical composition further comprises HPBC in a range of from about 0.1% to about 20%, from about 1% to about 15%, or from about 2.5% to about 10%. The amount of solubility enhancer employed may depend on the amount of the compound in the composition.

### EXPERIMENTAL

### Preparation of Compounds

### General Synthetic Methods

As disclosed herein, general methods for preparing the compounds of Formulae I-XI or a form thereof as described herein can be prepared using the methods summarized in Schemes A-I by the suitable selection of reagents with appropriate substitution, solvents, temperatures, pressures, and other reaction conditions readily selected by one of ordinary skilled in the art. Many of the starting materials are commercially available or, when not available, can be prepared via standard, well-known synthetic methodology or using the routes described below using techniques known to those skilled in the art. The synthetic schemes provided herein comprise multiple reaction steps, each of which is intended to stand on its own and can be carried out with or without any preceding or succeeding step(s). In other words, each of the individual reaction steps of the synthetic schemes provided herein in isolation is contemplated.

Depending on the nature of the groups Ar, A, A′, Q, X₁, X₂, Y, Z, R₁, R₂ and R_{4b}, depicted in the schemes below, the final compounds or their precursors may be further elaborated using the standard, well-known synthetic methods such as SNAr displacement reaction, metal catalyzed coupling reactions like Suzuki coupling, Negishi coupling and Buchwald coupling, reductive amination, etc. to afford the compounds of the general Formulae I-XI.

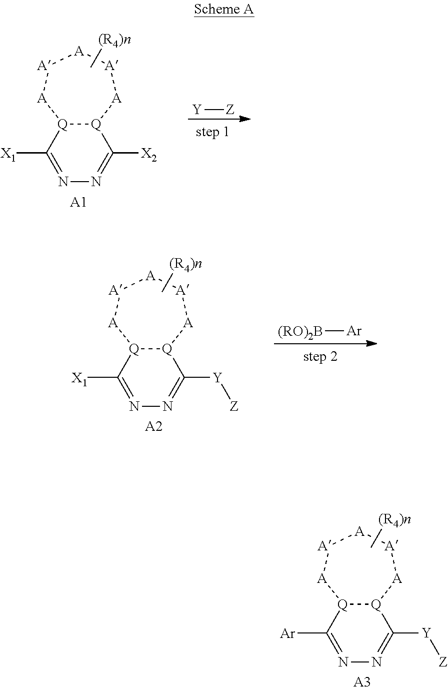

Compound A1 (where X₁ and X₂ are independently bromine, chlorine and the like) is converted to Compound A2 by a nucleophilic substitution with either an appropriate amine in the presence of a suitable base (such as DIEPA and the like) in a suitable solvent (such as NMP and the like) or with an appropriate alcohol in the presence of a suitable base (such as NaH and the like) in a suitable solvent (such as anhydrous THE and the like). Compound A2 is converted to Compound A3 by a Suzuki coupling with an aryl- or heteroaryl-boronic acid (or pinacol boronic ester) in the presence of a catalyst (such as Pd(dppf)Cl₂ and the like) and base (such as aqueous K₂CO₃ and the like) in a suitable solvent (such as 1,4-dioxane and the like).

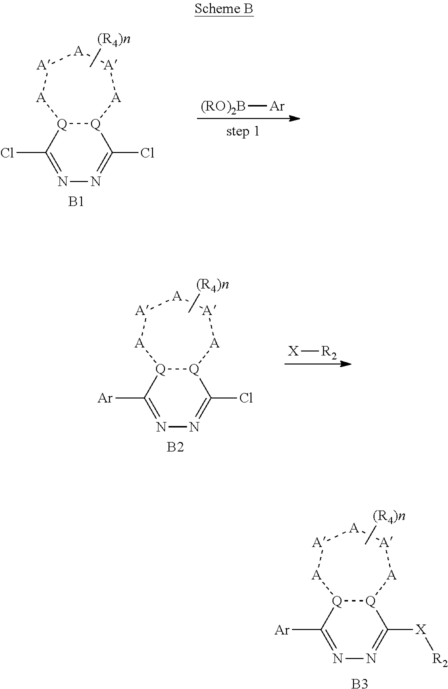

Compound B1 is converted to Compound B2 by a Suzuki coupling with an aryl- or heteroaryl-boronic acid (or pinacol boronic ester) in the presence of a catalyst (such as Pd(dppf)Cl₂ and the like) and base (such as aqueous K₂CO₃ and the like) in a suitable solvent (such as 1,4-dioxane and the like). Compound B2 is converted to Compound B3 by a Buchwald-Hartwig coupling with an appropriate amine in the present of a catalyst (such as Pd₂(dba)₃ and the like), a ligand (such as RuPhos and the like) and a base (such as NaO^{t}Bu and the like) in a suitable solvent (such as PhMe and the like).

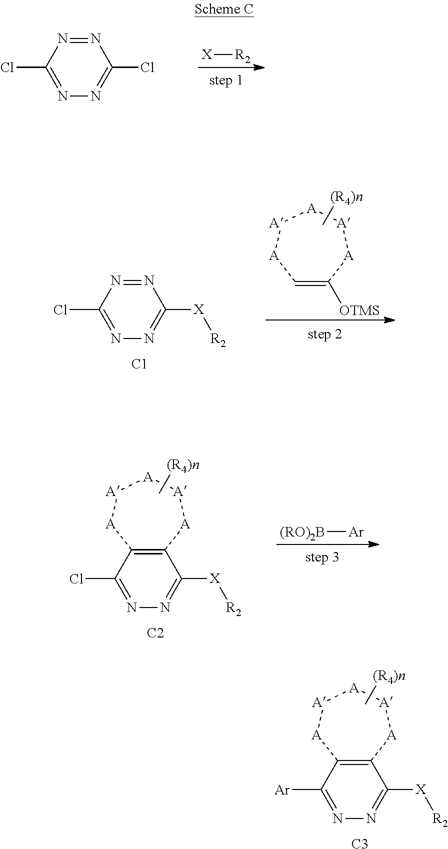

Compound C1 was prepared from 1,2,4,5-tetrazines with an appropriate amine in the presence of a suitable base (such as DIEPA and the like) in a suitable solvent (such as DCM and the like). Compound C1 was converted to Compound C2 by inverse electron demand Diels-Alder reaction with an appropriate enol ethers or enamine in a suitable solvent (such as PhMe and the like). Following conditions described in Scheme A—step 2, compound C2 was converted to compound C3.

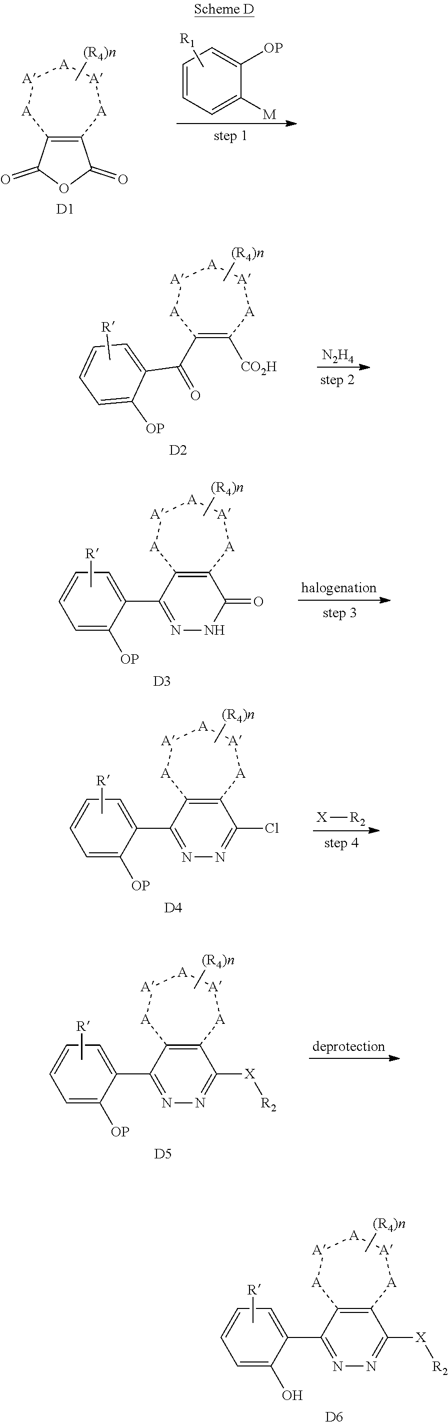

Compound D1 is converted to Compound D2 (where P is a protecting group such as Me and the like) by reacting with an appropriate organometallic compound (such as Grignard reagent and the like) in a suitable solvent (such as THE and the like). Compound D2 is converted to Compound D3 by condensation/cyclization sequence in the present of hydrazine in a suitable solvent (such as EtOH and the like). Compound D3 is converted to Compound D4 by treatment with a dehydrative halogenating agent (such as POCl₃ and the like). Compound D4 is converted to Compound D5 by a Buchwald-Hartwig coupling with an appropriate amine in the present of a catalyst (such as Pd₂(dba)₃ and the like), a ligand (such as RuPhos and the like) and a base (such as NaO^{t}Bu and the like) in a suitable solvent (such as PhMe and the like). Compound D5 is converted to Compound D6 upon treatment with conditions appropriate to removal of the protection groups (such as BBr₃ in DCM for a Me protection group in suitable solvent (such as DCM and the like).

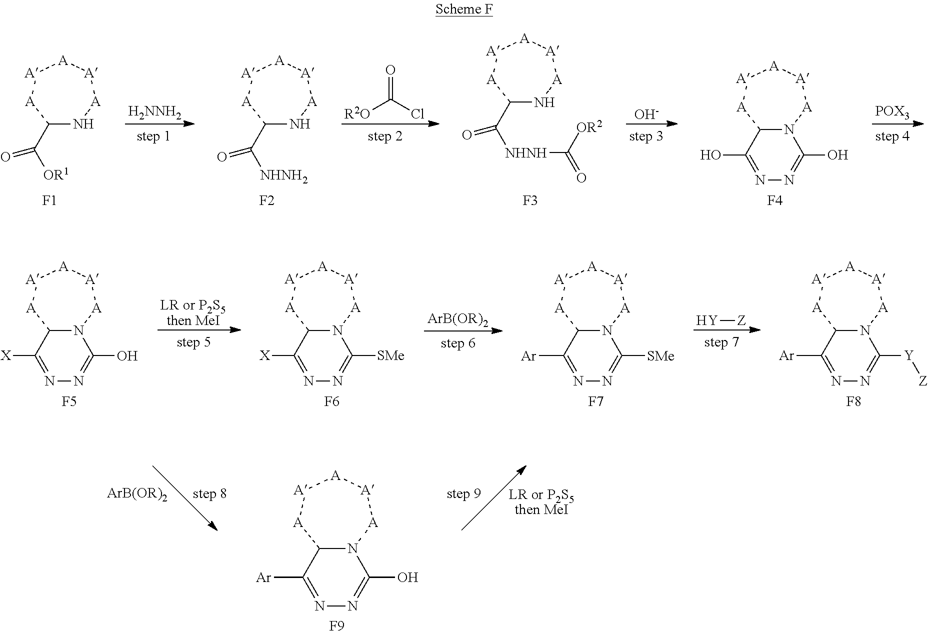

Compound F1 is converted to compound F2 by reacting with hydrazine in a suitable solvent (such as EtOH and the like). Reaction of F2 with chloroformate in the presence of a base (such as DIPEA and the like) in a suitable solvent (such as DCM and the like) provides F3, which is cyclized to F4 by treating with a base (such as KOH and the like) in a suitable solvent (such as EtOH and the like) at an elevated temperature (such as 80° C. and the like). Compound F4 is converted to compound F5 by treating with POX₃ (X═Cl or Br) with or without a base (such as DIPEA and the like). Treatment of F5 with a thionating reagent such as Lawesson's Reagent (LR) or P₂S₅ at an appropriate temperature such as 100° C., followed by alkylation with MeI provides F6. Compound F6 is converted to F7 by Suzuki coupling with an aryl or hetero boronic acid or borate in the presence of a suitable catalyst (such as PdCl₂ dppf and the like) and a base (such as K₂CO₃ and the like) in a suitable solvent (such as dioxane and the like). Alternatively, compound F5 is converted to compound F7 by a Suzuki coupling first to give compound F9, followed by thionation with LR or P₂S₅ and subsequent alkylation with MeI. SNAr reaction of F7 with a nucleophile in a suitable solvent (such as DMSO and the like) at an elevated temperature (such as 130° C. and the like) provides F8.

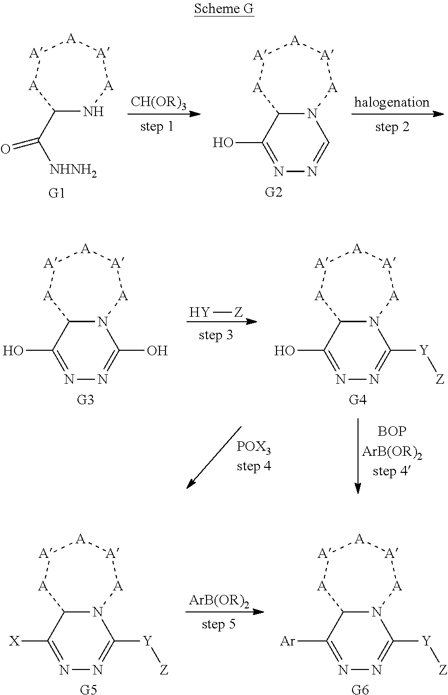

Compound G1, prepared according to step 1 in scheme F, is converted to compound G2 by reacting with tri-alkoxy orthoformate in a suitable solvent (such as EtOH and the like) at an elevated temperature (such as 100° C. and the like). Reaction of G2 with a halogenation reagent (such as NBS and the like) in a suitable solvent (such as DMF and the like) provides G3, which is reacted with a nucleophile to give the compound G4. Compound G4 is converted to compound G5 by treating with POX₃ (X═Cl or Br) with or without a base (such as DIPEA and the like) at an elevated temperature (such as 100° C. and the like). Suzuki coupling of compound G5 with an aryl or heteroaryl boronic acid or borate in the presence of a suitable catalyst (such as PdCl₂ dppf and the like) and a base (such as K₂CO₃ and the like) in a suitable solvent (such as dioxane and the like) provides G6. Alternatively, compound G4 can be converted to compound G6 directly by a BOP-medicated Suzuki coupling.

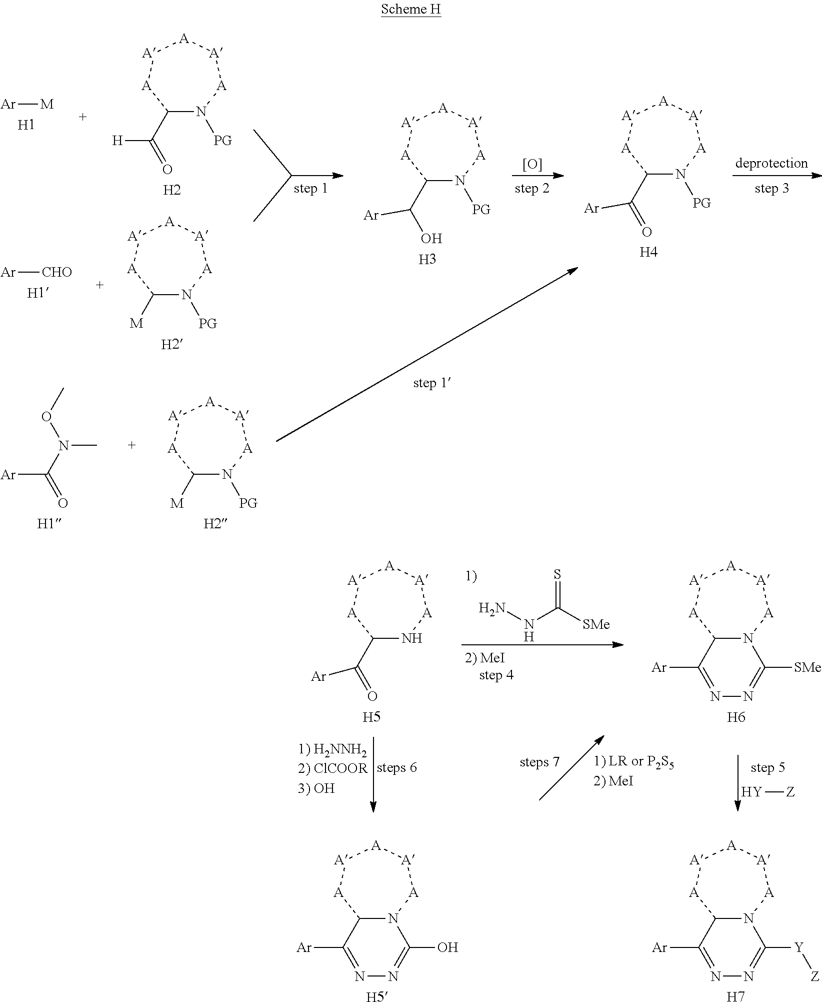

Reaction of an organometallic compound with an aldehyde, either H1 with H2, or H1′ with H2′, affords the alcohol H3. Compound H3 is converted to H4 by treating with an oxidant (such as MnO₂ and the like) in a suitable solvent (such as DCM and the like). Alternatively, reaction of compounds H1″ with H2″ yields H4 directly. Deprotection of compound H4 provides compound H5. Reaction of compound H5 with methyl hydrazinecarbodithioate in a suitable solvent (such as EtOH and the like) at an elevated temperature (such as 80° C. and the like) followed by alkylation with MeI in the presence of a base (such as K₂CO₃ and the like) provides compound H6. Alternatively, compound H5 is converted to compound H5′ by reacting with hydrazine in a suitable solvent (such as EtOH and the like), followed by reaction with chloroformate in the presence of a base (such as DIPEA and the like) in a suitable solvent (such as DCM and the like) and cyclization by treating with a base (such as KOH and the like) in a suitable solvent (such as EtOH and the like) at an elevated temperature (such as 80° C. and the like). SNAr reaction of H6 with a nucleophile in a suitable solvent (such as DMSO and the like) at an elevated temperature (such as 130° C. and the like) provides H7.

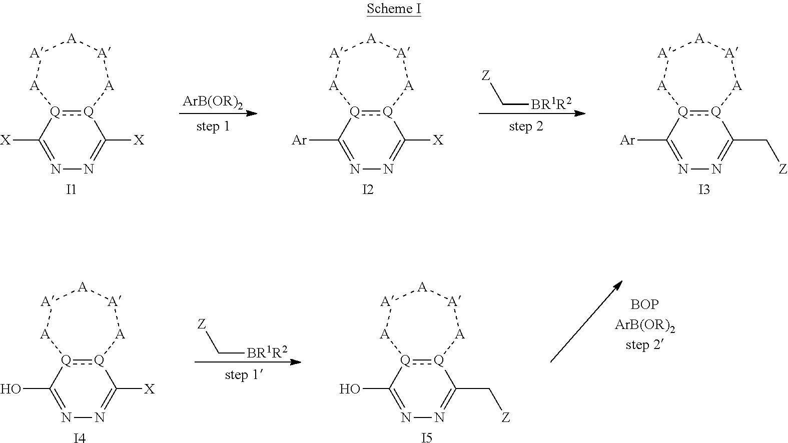

Compound I1 is converted to compound I3 by two coupling reactions with boronic acids or borates in the presence of a suitable catalyst (such as PdCl₂ dppf and the like) and a base (such as K₂CO₃ and the like) in a suitable solvent (such as dioxane and the like). Alternatively, compound I4 is converted to compound I5 by coupling with a boronic acid or borate, which is further converted to compound I3 by a BOP-mediated Suzuki coupling with an aryl or heteroaryl boronic acid or borate.

### Synthetic Examples

To describe in more detail and assist in understanding, the following non-limiting examples are offered to more fully illustrate the scope of compounds described herein and are not to be construed as specifically limiting the scope thereof. Such variations of the compounds described herein that may be now known or later developed, which would be within the purview of one skilled in the art to ascertain, are considered to fall within the scope of the compounds as described herein and hereinafter claimed. These examples illustrate the preparation of certain compounds. Those of skill in the art will understand that the techniques described in these examples represent techniques, as described by those of ordinary skill in the art, that function well in synthetic practice, and as such constitute preferred modes for the practice thereof. However, it should be appreciated that those of skill in the art should, in light of the present disclosure, appreciate that many changes can be made in the specific methods that are disclosed and still obtain a like or similar result without departing from the spirit and scope of the present description.

Other than in the following examples of the embodied compounds, unless indicated to the contrary, all numbers expressing quantities of ingredients, reaction conditions, experimental data, and so forth used in the specification and claims are to be understood as being modified by the term “about”. Accordingly, all such numbers represent approximations that may vary depending upon the desired properties sought to be obtained by a reaction or as a result of variable experimental conditions. Therefore, within an expected range of experimental reproducibility, the term “about” in the context of the resulting data, refers to a range for data provided that may vary according to a standard deviation from the mean. As well, for experimental results provided, the resulting data may be rounded up or down to present data consistently, without loss of significant figures. At the very least, and not as an attempt to limit the application of the doctrine of equivalents to the scope of the claims, each numerical parameter should be construed in light of the number of significant digits and rounding techniques used by those of skill in the art.

While the numerical ranges and parameters setting forth the broad scope of the present description are approximations, the numerical values set forth in the examples set forth below are reported as precisely as possible. Any numerical value, however, inherently contains certain errors necessarily resulting from the standard deviation found in their respective testing measurements.

### Compound Examples

As used above, and throughout the present description, the following abbreviations, unless otherwise indicated, shall be understood to have the following meanings:

| Abbreviation | Meaning |
|---|---|
| 9-BBN | 9-borabicyclo[3.3.1]nonane |
| AcOH or | acetic acid |
| HOAc | |
| ACN or MeCN | acetonitrile |
| Ar | argon gas |
| aq. | aqueous |
| BBr₃ | boron tribromide |
| BippyPhos | 5-(Di-tert-butylphosphino)-1′,3′,5′-triphenyl-1′H- |
| | [1,4′]bipyrazole |
| BOP | benzotriazol-1-yloxytris(dimethylamino)phosphonium |
| | hexafluorophosphate |
| Br₂ | bromine |
| nBuLi | n-butyl lithium |
| CaCO₃ | calcium carbonate |
| Cbz-Cl | benzyl chloroformate or benzyl chlorocarbonate |
| | or Z-chloride |
| CDCl₃ | deuterated chloroform or chloroform-d |
| Celite | diatomaceous earth |
| CH₂Cl₂ | dichloromethane |
| or DCM | |
| m-CPBA | meta-chloroperoxybenzoic acid |
| Cs₂CO₃ | cesium carbonate |
| CO | carbon monoxide |
| CuI | copper(I) iodide |
| DAST | diethylaminosulfur trifluoride |
| DBU | 1,8-diazabicyclo[5.4.0]undec-7-ene |
| DCE | 1,2-dichloroethane or ethylene dichloride |
| DIEA or | N,N-diisopropylethylamine or Hünig′s base |
| DIPEA or | |
| iPr₂NEt | |
| DMA | dimethylacetamide |
| DMF | dimethyl formamide |
| DMAP | 4-dimethylaminopyridine |
| DMSO | dimethylsulfoxide |
| EtOAc or EA | ethyl acetate |
| EtOH | ethanol |
| EZ-Prep | CombiFlash ® EZ Prep System by Teledyne ISCO |
| HATU | hexafluorophosphate azabenzotriazole tetramethyl |
| | uronium |
| HBr | hydrobromic acid |
| HBPin | pinacolborane |
| HCl | hydrochloric acid |
| (HCHO)n | formaldehyde |
| HPLC | high performance liquid chromatography |
| h, hr, min, s | hour (h or hr), minute (min), second (s) |
| iPrOH | isopropyl alcohol |
| K₂CO₃ | potassium carbonate |
| KOAc | potassium acetate |
| KOH | potassium hydroxide |
| Lawesson′s | 2,4-Bis-(4-methoxyphenyl)-1,3-dithia-2,4- |
| reagent | diphosphetane 2,4-disulfide |
| LC/MS, LCMS | liquid chromatographic mass spectroscopy |
| or LC-MS | |
| LDA | lithium diisopropylamide |
| M | molarity |
| MeI | iododmethane |
| MeOH | methanol |
| MOM-Br | Bromomethyl methyl ether |
| MS | mass spectroscopy |
| MsCl | methanesulfonyl chloride |
| Mg₂SO₄ | magnesium sulfate |
| N₂ | nitrogen gas |
| NaBH(OAc)₃ | sodium triacetoxyborohydride and sodium |
| | triacetoxyhydroborate |
| NaH | sodium hydride |
| NaHCO₃ | sodium bicarbonate |
| NaNO₂ | sodium nitrite |
| NaOH | sodium hydroxide |
| Na₂SO₄ | sodium sulfate |
| NH₃ | ammonia |
| NH₄HCO₃ | ammonium bicarbonate |
| NH₄OH | ammonium hydroxide |
| N₂H₄H₂O | hydrazine hydrate |
| NMP | N-methylpyrrolidone |
| NMR | nuclear magnetic resonance |
| Pd(OAc)₂ | palladium(II) acetate |
| Pd/C^{o} | palladium on carbon |
| Pd₂(dba)₃ | tris(dibenzylideneacetone)dipalladium(0) |
| or Pd₂dba₃ | |
| Pd(dppf)Cl₂ | [1,1′-bis(diphenylphosphino)ferrocene] |
| | dichloropalladium(II) |
| PE | petroleum ether |
| PhMe | toluene |
| POBr₃ | phosphoryl bromide or phosphorus oxybromide |
| prep-HPLC | preparative high performance liquid chromatography |
| RT or R.T. | room temperature |
| or rt | |
| RuPhos | 2-Dicyclohexylphosphino-2′,6′-diisopropoxybiphenyl |
| sat. | saturated |
| SFC | supercritical fluid chromatography |
| SGC | solvating gas chromatography |
| SiO₂ | silicon dioxide or silica gel |
| TBAF | tetra-n-butylammonium fluoride |
| t-Bu | tert-butyl |
| TEA, NEt₃, Et₃N | triethylamine |
| TFA | trifluoroacetic acid |
| THF | tetrahydrofuran |
| TLC | thin layer chromatography |
| UPLC | Ultra Performance Liquid Chromatography |
| Pd XPhos G3 | (2-dicyclohexylphosphino-2′,4′,6′-triisopropyl- |
| | 1,1′-biphenyl)[2-(2′-amino-1,1′- |
| | biphenyl)]palladium(II) methanesulfonate |
| Pd XPhos G4 | methanesulfonato(2-dicyclohexylphosphino-2′,4′,6′- |
| | tri-i-propyl-1,1′-biphenyl)(2′-methylamino- |
| | 1,1′-biphenyl-2-y1)palladium(II) |

### Intermediate 1a: 4-Chloropyrido[3,4-d]pyridazin-1(2H)-one

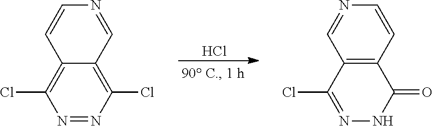 [SMILES: Clc1nnc(Cl)c2cnccc12 ; Cl ; O=c1[nH]nc(Cl)c2cnccc12]

To a solution of 1,4-dichloropyrido[3,4-d]pyridazine (4 g, 20.0 mmol) in water (40 mL) was added concentrated HCl (8 mL, 12 mol/L). The reaction mixture was stirred at 90° C. for 1 h. After cooling, the precipitate (1.3 g) formed was filtered off and crystallized by acetic acid to give 4-chloropyrido[3,4-d]pyridazin-1(2H)-one (1 g, 5.5 mmol, 27%) as a white powder. ¹H NMR (400 MHz, DMSO-d6) δ 13.15 (s, 1H), 9.32 (d, J=0.6 Hz, 1H), 9.10 (d, J=5.2 Hz, 1H), 8.12 (dd, J=5.2, 0.8 Hz, 1H).

### Intermediate 1b: 1-Bromopyrido[3,4-d]pyridazin-4(3H)-one

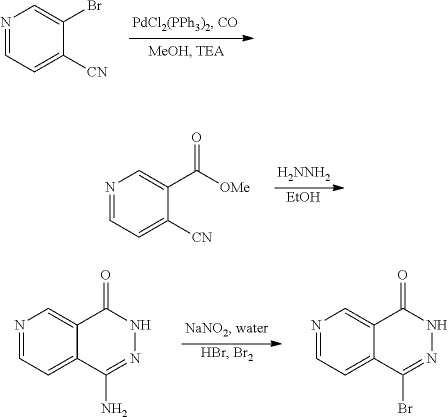

### Step 1. Methyl 4-cyanonicotinate

A mixture of 3-bromopyridine-4-carbonitrile (5 g, 27 mmol), TEA (45 mL), Pd(dppf)Cl₂ (1.95 g, 2.68 mmol) in MeOH (40 mL) was stirred for 16 hours at 80° C. under CO (40 MPa). The reaction mixture was concentrated under reduced pressure and the residue was purified by column chromatography (SiO₂, Petroleum ether/Ethyl acetate=100/1 to 5/1) to afford methyl 4-cyanonicotinate (2.8 g, 63% Yield) as a white solid. MS m/z 163.2 [M+H]⁺.

### Step 2. 1-Aminopyrido[3,4-d]pyridazin-4(3H)-one

To a mixture of methyl 4-cyanonicotinate (2.8 g, 17 mmol) in MeOH (28 mL) was added hydrazine (6.9 g, 140 mmol). The resulting mixture was stirred at 70° C. for 16 h. After cooling to rt, the mixture was filtered and the solid was washed with methanol and dried under vacuum to give 1-aminopyrido[3,4-d]pyridazin-4(3H)-one (2.7 g, 96% Yield) as a white solid. ¹H NMR (400 MHz, DMSO-d₆) δ 11.82 (s, 1H), 9.42 (s, 1H), 8.96 (d, J=5.2 Hz, 1H), 8.03 (d, J=5.2 Hz, 1H), 6.27 (s, 2H).

### Step 3. 1-Bromopyrido[3,4-d]pyridazin-4(3H)-one

To a solution of 1-aminopyrido[3,4-d]pyridazin-4(3H)-one (8 g, 49.3 mmol) at 0° C. was added 40% aqueous HBr (120 mL) dropwise over 10 min. A solution of NaNO₂ (8.8 g, 128 mmol) in water (40 mL) was then added dropwise over 1 h and the mixture was stirred at 0° C. for 5 min. Br₂ (1.48 g, 9.25 mmol) was then added portion wise over 30 min and the mixture was heated at 70° C. for 1 h. Water (80 mL) was added and the resulting precipitate was collected by filtration, washed with water and dried under vacuum to give the title compound (7.0 g, 63%) as a white solid. MS m/z 226.1, 228.1 [M+H]⁺; ¹H NMR (400 MHz, DMSO-d₆) δ 13.26 (s, 1H), 9.45-9.38 (m, 1H), 9.15-9.10 (m, 1H), 7.79-7.73 (m, 1H).

### Intermediate 2a: 1-Chloro-7-methylpyrrolo[1,2-d][1,2,4]triazin-4(3H)-one

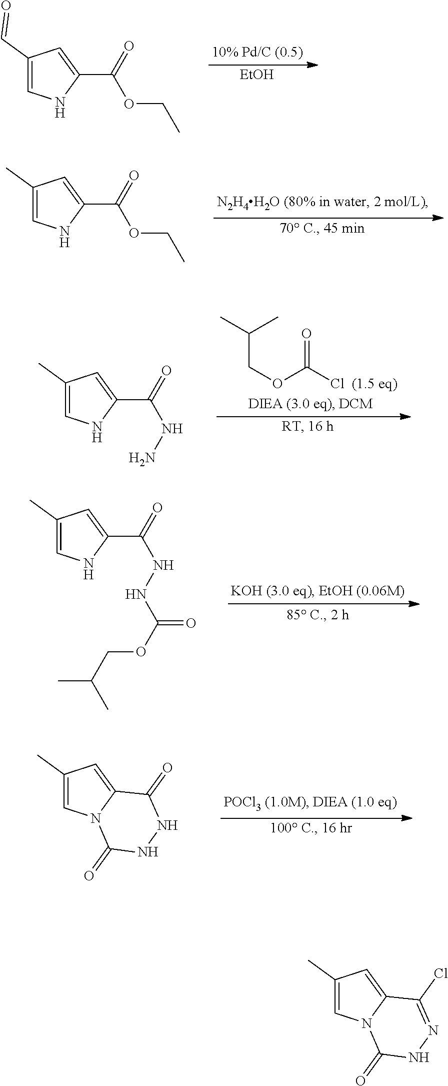

### Step 1. Ethyl 4-methyl-1H-pyrrole-2-carboxylate

A mixture of ethyl 4-formyl-1H-pyrrole-2-carboxylate (10.3 g, 61.6 mmol, 1.0 eq.) and Pd/C (10%, 2.0 g) in EtOH (150 mL) was stirred at rt overnight under hydrogen (balloon). The mixture was filtered. The filtrate was concentrated under reduced pressure. The crude colorless oil (8.50 g, 55.5 mmol, 90% yield) was used in next step without further purification. MS m/z 154.2 [M+H]⁺.

### Step 2. 4-Methyl-1H-pyrrole-2-carbohydrazide

A solution of ethyl 4-methyl-1H-pyrrole-2-carboxylate (8.50 g, 55.5 mmol) in N₂H₄H₂O (80%, 28 mL) was heated at 70° C. for 45 min. After cooling to rt, the mixture was filtered, the filter cake was washed with EtOH and dried in vacuo to give 4-methyl-1H-pyrrole-2-carbohydrazide as a white solid (7.5 g, 97% yield). MS m/z 140.2 [M+H]⁺. ¹H NMR (400 MHz, DMSO-d6) δ: 11.07 (s, 1H), 9.14 (s, 1H), 6.62 (s, 1H), 6.55 (s, 1H), 4.27 (s, 2H), 2.00 (s, 3H).

### Step 3. Isobutyl 2-(4-methyl-1H-pyrrole-2-carbonyl) hydrazine-1-carboxylate

To a suspension of 4-methyl-1H-pyrrole-2-carbohydrazide (7.5 g, 53.9 mmol, 1.0 eq.) and DIEA (20.9 g, 161 mmol, 3.0 eq.) in DCM (540 mL) was added isobutyl carbonochloridate (11.1 g, 80.9 mmol, 1.5 eq.) dropwise with ice-water cooling bath. The mixture was stirred at rt overnight. Upon completion, the reaction mixture was diluted with DCM, washed with water and brine. The organic phase was dried over Na₂SO₄, filtered, and concentrated under reduced pressure. The crude residue was purified by silica gel column chromatography eluting with 0-30% EtOAc in hexanes to afford isobutyl 2-(4-methyl-1H-pyrrole-2-carbonyl) hydrazine-1-carboxylate (12.0 g, 93%) as a white solid. MS m/z 240.2 [M+H]⁺.

### Step 4. 7-Methyl-2,3-dihydropyrrolo[1,2-d][1,2,4]triazine-1,4-dione

To a solution of isobutyl 2-(4-methyl-1H-pyrrole-2-carbonyl) hydrazine-1-carboxylate (12.0 g, 50.2 mmol, 1.0 eq.) in EtOH (840 mL) was added KOH (8.40 g, 151 mmol, 3.0 eq.). The mixture was stirred at 85° C. for 2 h. The reaction mixture was cooled to room temperature and filtered, the filter cake was washed with EtOH. The solid was dissolved with water, and adjusted to pH=4 with 1M HCl. The precipitate was filtered, the filter cake was washed with water and dried in vacuo to afford 7-methyl-2,3-dihydropyrrolo[1,2-d][1,2,4]triazine-1,4-dione (4.0 g, 48% yield) as a white solid. MS m/z 166.2 [M+H]⁺.

### Step 5. 1-Chloro-7-methylpyrrolo[1,2-d][1,2,4]triazin-4(3H)-one

To a solution of 7-methyl-2,3-dihydropyrrolo[1,2-d][1,2,4]triazine-1,4-dione (4.0 g, 24.2 mmol, 1.0 eq.) in POCl₃ (1.0 M, 25 mL) was added DIEA (3.12 g, 24.2 mmol, 1.0 eq.) dropwise with ice cooling bath. The mixture was stirred at 100° C. for 16 h. The reaction mixture was cooled to room temperature, concentrated. The residue was carefully poured into ice water, adjusted pH=8 with sat NaHCO₃ aq. The mixture was extracted with DCM: MeOH (10:1, 100 mL×3), dried over Na₂SO₄, concentrated, purified by silica gel column chromatography eluting with 0-50% EtOAc in hexanes to afford 1-chloro-7-methylpyrrolo[1,2-d][1,2,4]triazin-4(3H)-one (2.0 g, 45% yield) as a white solid. MS m/z 184.3 [M+H]⁺. ¹H NMR (400 MHz, DMSO-d6) δ: 12.43 (s, 1H), 7.66 (s, 1H), 6.75 (s, 1H), 2.23 (s, 3H).

### Intermediate 2b: 8-Fluoro-2,3-dihydropyrrolo[1,2-d][1,2,4]triazine-1,4-dione

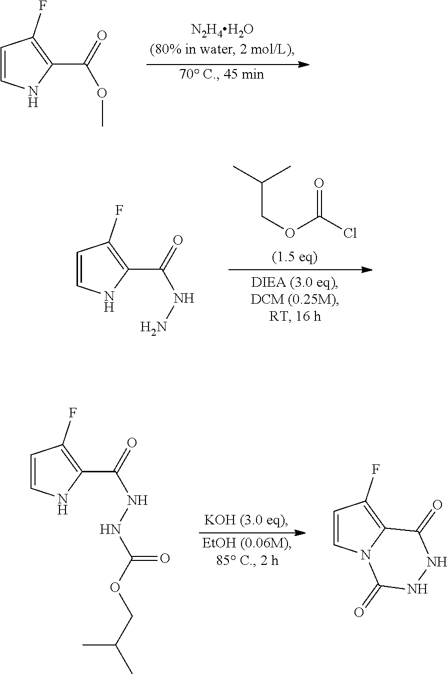

### Step 1. 3-Fluoro-1H-pyrrole-2-carbohydrazide

A mixture of N₂H₄-H₂O (80% in water, 18 mL) and methyl 3-fluoro-1H-pyrrole-2-carboxylate (3.14 g, 20.0 mmol) was stirred at 70° C. for 45 minutes. After cooling to rt, the precipitate was filtered, the filter cake was washed with water and dried under vacuum to obtain 3-fluoro-1H-pyrrole-2-carbohydrazide (2.30 g, 73%) as a white solid. MS m/z 144.1 [M+H]⁺, ¹H NMR (400 MHz, DMSO-d6) δ: 11.31 (s, 1H), 8.55 (s, 1H), 6.74 (dd, J=4.6, 3.0 Hz, 1H), 5.97 (d, J=3.0 Hz, 1H), 4.39 (s, 2H).

### Step 2. Isobutyl 2-(3-fluoro-1H-pyrrole-2-carbonyl)hydrazine-1-carboxylate

To a solution of 3-fluoro-1H-pyrrole-2-carbohydrazide (2.15 g, 15.0 mmol, 1.0 eq.) in DCM (60 mL) was added DIPEA (5.80 g, 45.0 mmol, 3.0 eq.). Isobutyl carbonochloridate (3.00 g, 22.5 mmol, 1.5 eq.) was added to the mixture slowly. The mixture was stirred at rt for 16 h. After concentration, the mixture was diluted with EtOAc (200 mL). The organic layer was washed with water and brine. The organic layer was dried over Na₂SO₄, filtered and concentrated under reduced pressure. The crude was purified by silica gel column chromatography eluting with 0-50% EtOAc in hexanes to afford isobutyl 2-(3-fluoro-1H-pyrrole-2-carbonyl)hydrazine-1-carboxylate (2.44 g, 66.7% yield) as yellow solid. MS m/z 244.1 [M+H]⁺, ¹H NMR (400 MHz, DMSO-d6) δ: 11.46 (s, 1H), 9.18 (s, 1H), 9.09 (s, 1H), 6.82 (d, J=4.4 Hz, 1H), 6.01 (d, J=2.5 Hz, 1H), 3.82 (d, J=6.6 Hz, 2H), 2.02-1.67 (m, 1H), 0.92 (d, J=6.7 Hz, 6H).

### Step 3. 8-Fluoro-2,3-dihydropyrrolo[1,2-d][1,2,4]triazine-1,4-dione

To a solution of isobutyl 2-(3-fluoro-1H-pyrrole-2-carbonyl)hydrazine-1-carboxylate (2.44 g, 10.0 mmol, 1.0 eq.) in EtOH (60 mL) was added KOH (1.68 g, 30 mmol, 3 eq.). The mixture was stirred at 85° C. for 2 h. After cooling to rt, the mixture was concentrated. The residue was diluted with water (50 mL) and adjusted to pH to 6-7 with 1M HCl. The precipitates were filtered, the filter cake was washed with water and dried under vacuum to obtain 8-fluoro-2,3-dihydropyrrolo[1,2-d][1,2,4]triazine-1,4-dione (1.16 g, 68.7% yield) as brown solid. MS m/z 168.0 [M−H]⁺, ¹H NMR (400 MHz, DMSO-d6) δ: 11.52 (s, 2H), 7.57 (t, J=4.1 Hz, 1H), 6.68 (d, J=3.4 Hz, 1H).

### Intermediate 2c: 8-Chloro-2,3-dihydropyrrolo[1,2-d][1,2,4]triazine-1,4-dione

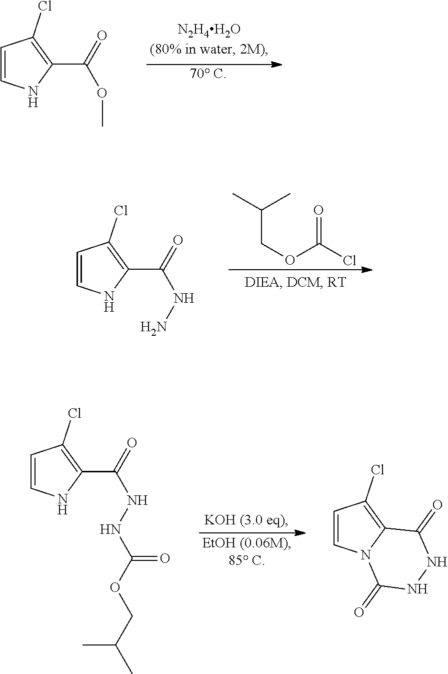

The title compound was prepared in analogous manner according to the procedure of Intermediate 2b, using methyl 3-chloro-1H-pyrrole-2-carboxylate in place of 3-fluoro-1H-pyrrole-2-carboxylate in step 1. MS m/z 186.0, 188.0 [M+H]⁺.

### Intermediate 2d: 1-chloro-4-(methylthio)pyrrolo[1,2-d][1,2,4]triazine

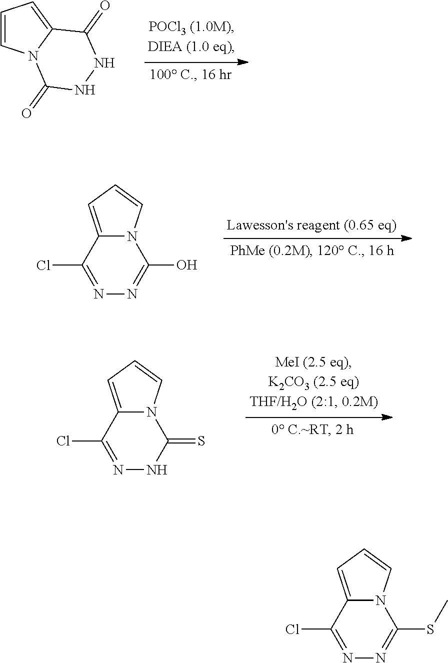

### Step 1. 1-Chloropyrrolo[1,2-d][1,2,4]triazin-4-ol

To a stirred solution of 2,3-dihydropyrrolo[1,2-d][1,2,4]triazine-1,4-dione (2 g, 13.2 mmol) in POCl₃ (60 mL) was added DIEA (1.84 g, 14.2 mmol). The reaction mixture was heated at 100° C. for 16 h. After cooling to rt, the mixture was concentrated under reduced pressure to give a residue. The residue was diluted with cold saturated sodium bicarbonate solution and extracted with dichloromethane (3×50 mL). The combined organic extracts were dried over sodium sulfate and evaporated under reduced pressure. The crude was further triturated with DCM to give the title compound 1-chloropyrrolo[1,2-d][1,2,4]triazin-4-ol (0.867 g, 38.6% Yield) as an off-white solid, which was used to next step without further purification. MS m/z 169.6 [M+H]⁺. ¹H NMR (400 MHz, DMSO-d6) δ 12.54 (s, 1H), 7.88-7.87 (m, 1H), 6.92-6.89 (m, 2H).

### Step 2. 1-Chloropyrrolo[1,2-d][1,2,4]triazine-4(3H)-thione

To a solution of 1-chloropyrrolo[1,2-d][1,2,4]triazin-4-ol (30.0 g, 177 mmol) in toluene (1200 mL) was added Lawesson's Reagent (46.2 g, 114 mmol). The reaction was stirred at 120° C. for 16 h. The mixture was diluted with H₂O (1.5 L), acidified with aqueous HCl (1 M) till pH=3 and extracted with EA. The EA layer was wash with sat. NaHCO₃ aqueous solution (1 L) and brine (1 L), then dried over anhydrous Na₂SO₄ and concentrated to give crude 1-chloro-3H-pyrrolo[1,2-d][1,2,4]triazine-4-thione (crude 35.0 g, overweight), which was used to next step without further purification. MS m/z 184.1 [M−H]⁻.

### Step 3. 1-Chloro-4-(methylthio)pyrrolo[1,2-d][1,2,4]triazine

To a solution of 1-chloro-3H-pyrrolo[1,2-d][1,2,4]triazine-4-thione (crude 35.0 g, 189 mmol) in THE (600 mL) and water (300 mL) was added K₂CO₃ (65.2 g, 472 mmol), followed by iodomethane (68.6 g, 483 mmol) at 0° C. The reaction mixture was stirred for 2 h at room temperature, then diluted with water and extracted with EA (3×100 mL). The combined organic layers were washed with water, brine, dried over anhydrous Na₂SO₄ and concentrated. The residue was purified by column chromatography (SiO₂, Petroleum ether/Ethyl acetate=50/1 to 20/1) to afford title product 1-chloro-4-methylsulfanyl-pyrrolo[1,2-d][1,2,4]triazine (21.8 g, 109 mmol, 57.9% for two steps Yield) as a white solid. MS m/z 200.0 [M+H]⁺, ¹H NMR (400 MHz, DMSO-d6) δ: 7.83 (dd, J=2.8 Hz, 1.2 Hz, 1H), 7.16 (dd, J=4.0 Hz, 2.8 Hz, 1H), 7.11 (dd, J=4.0 Hz, 1.2 Hz, 1H), 2.81 (s, 3H).

The intermediates below were prepared according to the procedure of Intermediate 2d by substituting the appropriate starting materials, reagents and reaction conditions.

| Structure | Spectral Data |
|---|---|
| 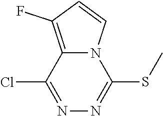 | From Intermediate 2b; ¹H NMR (400 MHz, DMSO-d6) δ: 7.73-7.71 (m, 1H), 7.14 (d, J = 3.2 Hz, 1H), 2.79 (s, 3H). |
| 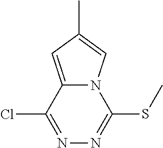 | From Intermediate 2a; ¹H NMR (400 MHz, DMSO-d6) δ: 7.65 (s, 1H), 6.94 (s, 1H), 2.79 (s, 1H), 2.33 (s, 1H). |

### Intermediate 2e: 1-Bromo-4-(methylthio)pyrrolo[1,2-d][1,2,4]triazine

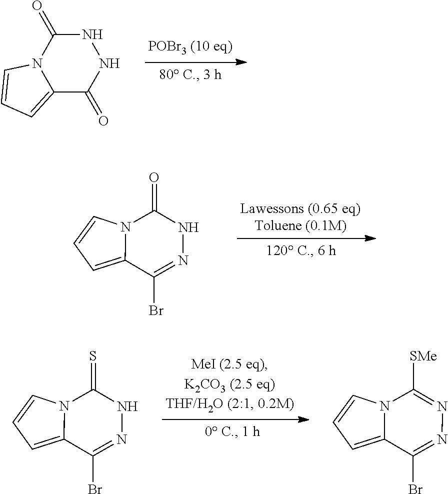 [SMILES: O=c1[nH][nH]c(=O)n2cccc12 ; O=c1[nH]nc(Br)c2cccn12 ; S=c1[nH]nc(Br)c2cccn12 ; CSc1nnc(Br)c2cccn12]

### Step 1. 1-Bromopyrrolo[1,2-d][1,2,4]triazin-4(3H)-one

A mixture of 2,3-dihydropyrrolo[1,2-d][1,2,4]triazine-1,4-dione (30.0 g, 0.199 mol, 1.0 eq.) and POBr₃ (570 g, 1.99 mol, 10 eq.) was heated at 80° C. for 3 hours. The hot reaction mixture was slowly pour into a stirring mixture of ice and aqueous sat. NaHCO₃. The mixture was neutralized with solid NaHCO₃ to pH ˜ 7 and extracted with a mixture of MeOH/DCM (1 L×2, 1:10). The combined organic phase was dried over Na₂SO₄, filtered, and concentrated under reduced pressure. The crude residue was triturated with DCM, filtered and washed with DCM. The solid was dried under vacuum to afford 1-bromopyrrolo[1,2-d][1,2,4]triazin-4(3H)-one (21.9 g, 0.103 mol, 51.8% yield) as a pink solid. MS m/z 214.0, 215.9 [M+H]⁺.

### Step 2. 1-Bromopyrrolo[1,2-d][1,2,4]triazine-4(3H)-thione

To a solution of 1-bromopyrrolo[1,2-d][1,2,4]triazin-4(3H)-one (10.0 g, 46.7 mmol, 1.0 eq.) in DMSO (472 mL) was added Lawesson's Reagent (12.4 g, 30.7 mmol, 0.65 eq.). The reaction mixture was heated at 120° C. for 6 hours. The reaction mixture was cooled to room temperature, diluted with aqueous saturated NaHCO₃ and extracted with EtOAc (300 mL×3). The organic layer was dried over Na₂SO₄, filtered and concentrated under reduced pressure. The crude was purified by silica gel column chromatography eluting with 0-30% EtOAc in hexanes to afford 1-bromopyrrolo[1,2-d][1,2,4]triazine-4(3H)-thione as a white solid (1.56 g, 6.78 mmol, 14.5% yield). MS m/z 228.0, 230.0 [M−H]⁻. ¹H NMR (400 Hz, DMSO-d6) δ:14.15 (s, 1H), 8.23 (dd, J=2.9, 1.4 Hz, 1H), 7.10 (dd, J=3.7, 3.1 Hz, 1H), 7.06 (dd, J=3.9, 1.4 Hz, 1H).

### Step 3. 1-Bromo-4-(methylthio)pyrrolo[1,2-d][1,2,4]triazine

To a mixture of 1-bromopyrrolo[1,2-d][1,2,4]triazine-4(3H)-thione (2.00 g, 8.69 mmol, 1.0 eq.) and K₂CO₃ (3.00 g, 21.7 mmol, 2.5 eq.) in THE (43 mL) and water (21 mL) was added iodomethane (1.35 mL, 2.28 g/mL, 21.7 mmol, 2.5 eq.) dropwise at 0° C. The reaction was stirred at 0° C. for 1 hour. The reaction mixture was diluted with water (50 mL) and extracted with EtOAc (50 mL×3). The organic layer was dried over Na₂SO₄, filtered and concentrated under reduced pressure. The crude product was purified by silica gel column chromatography eluting with 0-30% EtOAc in hexanes to afford 1-bromo-4-(methylthio)pyrrolo[1,2-d][1,2,4]triazine (1.40 g, 5.74 mmol, 66.1% yield) as a white solid. MS m/z 243.9, 246.0 [M+H]⁺. ¹H NMR (400 Hz, DMSO-d6) δ: 7.83 (dd, J=2.8, 1.0 Hz, 1H), 7.15 (dd, J=4.0, 2.8 Hz, 1H), 7.03 (dd, J=4.0, 1.0 Hz, 1H), 2.79 (s, 3H).

### Intermediate 3: 1-Chloroimidazo[1,5-d][1,2,4]triazin-4-ol

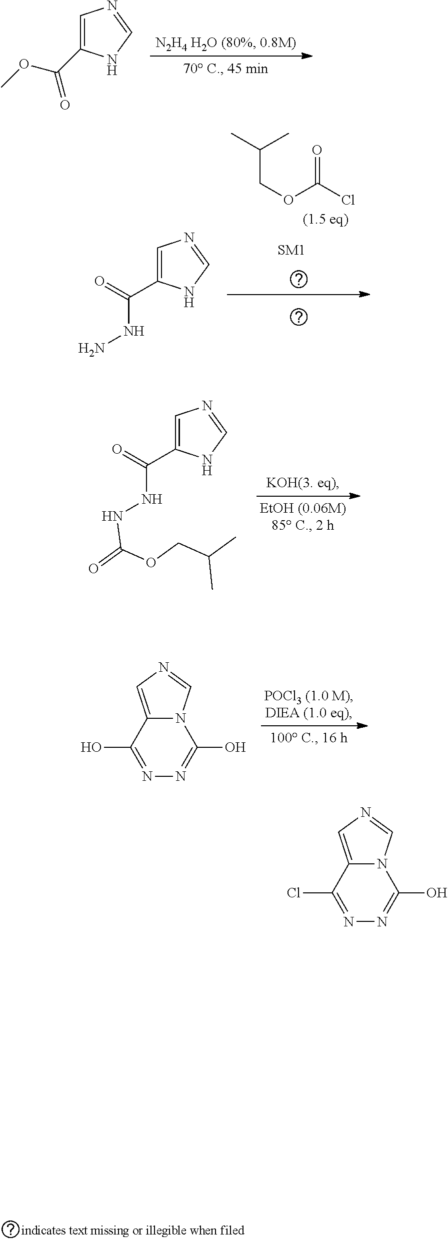

### Step 1. 1H-Imidazole-5-carbohydrazide

A solution of methyl 1H-imidazole-5-carboxylate (21 g, 166.5 mmol) in hydrazinium hydroxide solution (80%, 260 mL) was stirred at 70° C. in sealed tube for 45 mins. After cooling to room temperature, it was concentrated in vacuo to get the crude product, which was triturated with ether and filtered to provide the title compound as white solids (19.9 g, yield 94.8%). MS m/z 127.2 [M+H]⁺.

### Step 2. Isobutyl 2-(1H-imidazole-5-carbonyl)hydrazine-1-carboxylate

To a solution of 1H-imidazole-5-carbohydrazide (19.9 g, 157.9 mmol) in DCM (200 mL) was added N,N-Diisopropylethylamine (61.1 g, 473.7 mmol) and isobutyl carbonochloridate (32.4 g, 236.9 mmol). The mixture was stirred rt for 16 h. After that, the reaction was quenched with water (500 mL), extracted with DCM (3×500 mL). The combined organic layers were concentrated in vacuo to get a crude. The crude was purified by column (DCM/MeOH=15:1) to provide the title compound as white solids (18.0 g, yield 50.4%). MS m/z 227.2 [M+H]⁺; ¹H NMR (400 MHz, CD₃OD) δ 7.72 (s, 2H), 3.90 (d, J=6.4 Hz, 2H), 2.01-1.81 (m, 1H), 0.95 (d, J=5.5 Hz, 6H).

### Step 3. Imidazo[1,5-d][1,2,4]triazine-1,4-diol

To a mixture of isobutyl 2-(1H-imidazole-5-carbonyl) hydrazine-1-carboxylate (1.0 g, 4.42 mmol) in ethanol (10 mL) was added KOH (743 mg, 13.26 mmol). The reaction mixture was heated at 85° C. for 16 h. After cooling to room temperature, it was concentrated in vacuo to get a crude. The crude was added con. HCl solution to adjust pH to 6 and extracted with (DCM/MeOH=10:1), the combined organic layers were dried over Na₂SO₄, filtered, and concentrated to get the title compound as brown solids (500 mg, yield 74.4%), which was used directly in the next step. MS m/z 153.2 [M+H]⁺.

### Step 4. 1-Chloroimidazo[1,5-d][1,2,4]triazin-4-ol

A solution of imidazo [1,5-d][1,2,4] triazine-1,4-diol (500 mg, 3.29 mmol) in Phosphorus oxychloride (5 mL) was added DIPEA (933 mg, 7.23 mmol) under ice bath. The mixture was stirred at 115° C. for 16 h. After cooling to room temperature, it was concentrated in vacuo to get a crude. The crude was added DCM (20 mL) and the solution was slowly poured into excess sodium bicarbonate ice water solution, and a large number of air bubbles were generated, the aqueous phase was extracted with DCM (3×100 mL). The combined organic layers were concentrated to obtain the crude product, the crude product was triturated with PE/EA (1:1) to provide the title compound as brown solid (120 mg crude). MS m/z 171.2 [M+H]⁺.

### Intermediate 4a: 7-Bromo-2-methylpyrazolo[1,5-d][1,2,4]triazin-4-ol

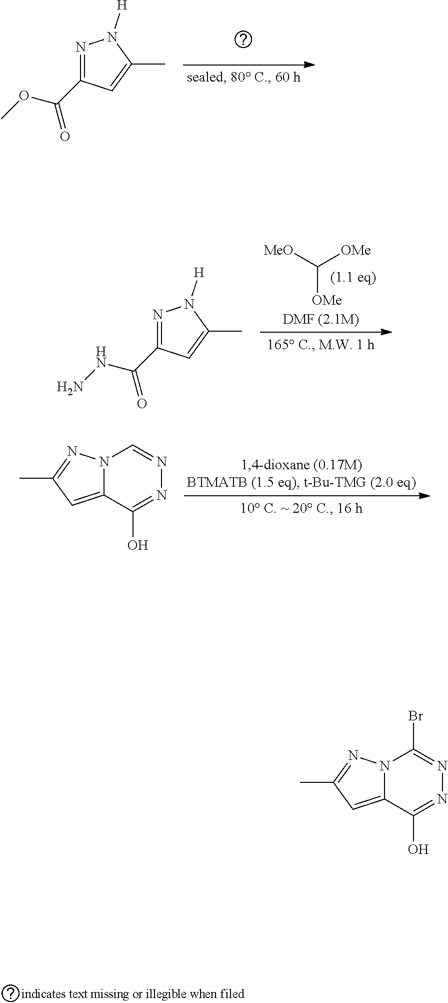

### Step 1. 5-Methyl-1H-pyrazole-3-carbohydrazide

Hydrazine hydrate (54 mL, 1.032 g/mL, 1.11 mol, 6.2 eq.) was added to a solution of methyl 5-methyl-1H-pyrazole-3-carboxylate (25.0 g, 0.178 mol, 1.0 eq.) in EtOH (250 mL). The mixture was sealed and heated at 80° C. for 60 h. The reaction mixture was cooled to room temperature. The solvent was evaporated in vacuo. The crude product was treated with a 1/1 mixture of water/MeOH, filtered and the filter cake was washed with water. The solid was dried under vacuum to afford 5-methyl-1H-pyrazole-3-carbohydrazide (21.7 g, 0.155 mol, 87.0% yield) as a white solid. MS m/z 141.1 [M+H]⁺.

### Step 2. 2-Methylpyrazolo[1,5-d][1,2,4]triazin-4-ol

A mixture of 5-methyl-1H-pyrazole-3-carbohydrazide (3.00 g, 21.4 mmol, 1.0 eq.) and trimethoxymethane (2.57 mL, 0.970 g/mL, 23.5 mmol, 1.1 eq.) in DMF (10 mL) was sealed in a tube. The reaction mixture was stirred at 165° C. for 1 hour under microwave then cooled to room temperature. The precipitate was collected by filtration, washed with EtOH and dried under vacuum to afford 2-methylpyrazolo[1,5-d][1,2,4]triazin-4-ol (1.70 g, 11.3 mmol, 52.9% yield) as a white solid. MS m/z 149.2 [M−H]⁻.

### Step 3. 7-Bromo-2-methylpyrazolo[1,5-d][1,2,4]triazin-4-ol

Benzyltrimethylammonium tribromide (26.5 g, 68.0 mmol, 1.5 eq.) was added in portions to a stirred solution of 2-methylpyrazolo[1,5-d][1,2,4]triazin-4-ol (6.80 g, 45.3 mmol, 1.0 eq.) and 2-(tert-butyl)-1,1,3,3-tetramethylguanidine (18.3 mL, 0.85 g/mL, 90.6 mmol, 2.0 eq.) in 1,4-dioxane (290 mL) under N₂ at 10° C. Once the addition was completed the mixture was warmed to 20° C. and stirred at this temperature for 16 hours. Then the mixture was quenched with a mixture of aqueous sat. Na₂S₂O₃ and aqueous sat. NaHCO₃ and then extracted with EtOAc (150 mL×3). The combined organic layers were dried over Na₂SO₄, filtered and concentrated. The crude product was triturated with EtOAc, filtered and washed with EtOAc. The solid was dried under vacuum to afford 7-bromo-2-methylpyrazolo[1,5-d][1,2,4]triazin-4-ol (7.60 g, 33.3 mmol, 73.6% yield) as an off-white solid. MS m/z 229.0, 230.9 [M+H]⁺. ¹H NMR (400 Hz, DMSO-d6) δ: 12.60 (s, 1H), 7.15 (s, 1H), 2.42 (s, 3H).

### Intermediate 4b: 7-Bromo-2-cyclopropylpyrazolo[1,5-d][1,2,4]triazin-4-ol

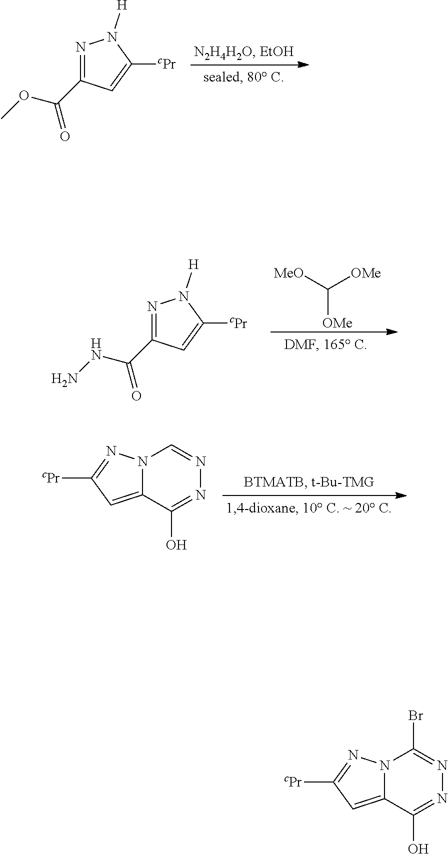

The title compound was prepared in analogous manner according to the procedure of Intermediate 4a, using methyl 5-cyclopropyl-1H-pyrazole-3-carboxylate in place of 5-methyl-1H-pyrazole-3-carboxylate in step 1. MS m/z 255.0, 257.0 [M+H]⁺; ¹H NMR (400 MHz, CDCl₃) δ 9.95 (bs, 1H), 6.94 (s, 1H), 2.15-2.17 (m, 1H), 1.12-1.15 (m, 2H), 0.92-0.94 (m, 2H).

### Intermediate 5a: 2-(2-(Methoxymethoxy)-4-(trifluoromethyl)phenyl)-4,4,5,5-tetramethyl-1,3,2-dioxaborolane

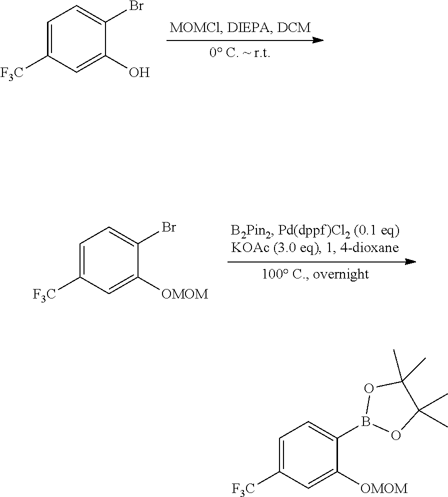

### Step 1. 1-Bromo-2-(methoxymethoxy)-4-(trifluoromethyl)benzene

To a cold solution of 2-bromo-5-(trifluoromethyl)phenol (2.20 g, 8.9 mmol) in DCM (7.2 mL) was added DIPEA (1.9 mL, 1.2 eq.), followed by addition of chloro(methoxy)methane (0.8 mL, 9.8 mmol, 1.1 eq.). The reaction mixture was warmed to rt, then stirred overnight. The reaction was quenched by ice water and extracted with EtOAc. The organics were washed with water and brine, dried over sodium sulfate and concentrated. The crude material was purified by flash column chromatography on silica gel eluting with 0-30% EtOAc in hexane to provide 1-bromo-2-(methoxymethoxy)-4-(trifluoromethyl)benzene (1.9 g, 77% yield). ¹H NMR (acetone-d6) δ: 7.70 (d, J=8.3 Hz, 1H), 7.38 (s, 1H), 7.13-7.19 (m, 1H), 5.29 (s, 2H), 3.37 (s, 3H).

### Step 2. 2-(2-(Methoxymethoxy)-4-(trifluoromethyl)phenyl)-4,4,5,5-tetramethyl-1,3,2-dioxaborolane

A mixture of 1-bromo-2-(methoxymethoxy)-4-(trifluoromethyl)benzene (1.06 g, 3.72 mmol, 1.0 eq.), bis(pinacolato)diboron (1.95 g, 7.4 mmol, 2.0 eq.), Pd(dppf)Cl₂ (153 mg, 0.05 eq.) and KOAc (1.13 g, 11 mmol, 3.0 eq.) in dioxane (12 mL) was heated at 100° C. under Ar for 16 hr. After cooling, the reaction mixture was diluted with DCM, filtered through celite and concentrated. The crude material was purified by flash column chromatography on silica gel eluting with 0-30% EtOAc in hexane to provide 2-[2-(methoxymethoxy)-4-(trifluoromethyl)phenyl]-4,4,5,5-tetramethyl-1,3,2-dioxaborolane (1.10 g, 89% yield). ¹H NMR (acetone-d6) δ: 7.81 (d, J=7.6 Hz, 1H), 7.33-7.39 (m, 2H), 5.31 (s, 2H), 3.51 (s, 3H), 1.28-1.39 (s, 12H).

The intermediates below were prepared according to the procedure of Intermediate 5a by substituting the appropriate starting materials, reagents and reaction conditions.

| Structure | Spectral Data |
|---|---|
| 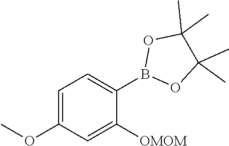 | ¹H NMR (400 MHz, DMSO-d6) δ 7.50 (d, J = 8.8 Hz, 1H), 6.61-6.56 (m, 2H), 5.15 (s, 2H), 3.75 (s, 3H), 3.41 (s, 3H), 1.26 (s, 12H). |
| 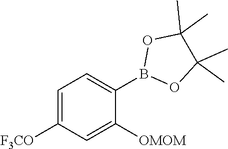 | ¹H NMR (400 MHz, DMSO-d6) δ: 7.65 (d, J = 8.4 Hz, 1H), 7.05-7.04 (m, 1H), 7.00-6.98 (m, 1H), 5.22 (s, 2H), 3.41 (s, 3H), 1.28 (s, 12H). |
| 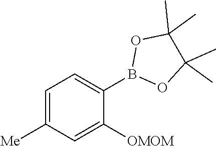 | ¹H NMR (400 MHz, DMSO-d6) δ: 7.46 (d, J = 7.6 Hz, 1H), 6.89 (s, 1H), 6.83 (d, J = 7.6 Hz, 1H), 5.14 (s, 2H), 3.41 (s, 3H), 2.30 (s, 3H), 1.27 (s, 12H). |
| 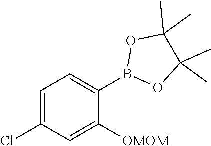 | ¹H NMR (400 MHz, CDCl₃) δ: 7.60 (t, J = 6.8 Hz, 1H), 7.05 (d, J = 1.8 Hz, 1H), 7.00 (dd, J = 8.0, 1.8 Hz, 1H), 5.19 (d, J = 8.6 Hz, 2H), 3.49 (d, J = 10.9 Hz, 3H), 1.34 (s, 12H). |

### Intermediate 5b: 3-(Methoxymethoxy)-4-(4,4,5,5-tetramethyl-1,3,2-dioxaborolan-2-yl)benzonitrile

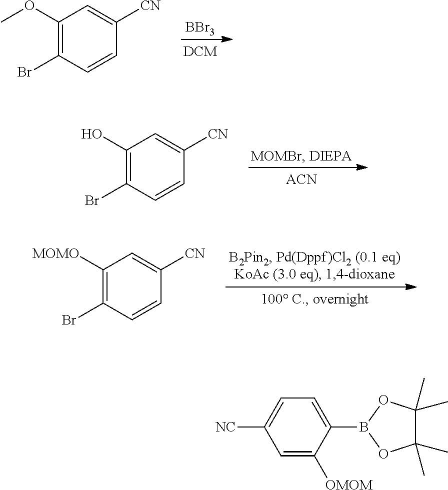

### Step 1. 4-Bromo-3-hydroxybenzonitrile

To a solution of 4-bromo-3-methoxybenzonitrile (5 g, 23.6 mmol) was added BBr₃ (1 M in DCM, 50 mL). The reaction was stirred at R.T for 16 h, monitored by TLC and LCMS. After reaction, solvent was removed and residue was purified via flash column chromatography to give 4-bromo-3-hydroxybenzonitrile (2.4 g, 51% Yield). MS m/z 198.0 [M+H]⁺.

### Step 2. 4-Bromo-3-(methoxymethoxy)benzonitrile

To a solution of 4-bromo-3-hydroxybenzonitrile (2.4 g, 12 mmol) in CH₃CN (24 mL) was added DIEA (7.8 g, 60 mmol) and MOM-Br (2 g, 16 mmol). The mixture was stirred at R.T. for 2 h, monitored by TLC and LCMS. After that, the mixture was poured into H₂O, extracted with EA, dried over Na₂SO₄ and concentrated. The residue was purified by flash column chromatography to give 4-bromo-3-(methoxymethoxy)benzonitrile (2 g, 70% Yield). MS m/z 242.0 [M+H]⁺.

### Step 3. 3-(Methoxymethoxy)-4-(4,4,5,5-tetramethyl-1,3,2-dioxaborolan-2-yl)benzonitrile

A solution of 4-bromo-3-(methoxymethoxy)benzonitrile (1 g, 4.13 mmol), bis(pinacolato)diboron (1.92 g, 4.92 mmol), potassium acetate (819 mg, 8.26 mmol), and Pd(dppf)Cl₂ (318 mg, 0.41 mmol) in 1,4-dioxane (10 mL) was stirred at 90° C. under N₂ for 16 h.

After reaction, the residue was purified by flash column chromatography to give 3-(methoxymethoxy)-4-(4,4,5,5-tetramethyl-1,3,2-dioxaborolan-2-yl)benzonitrile (1.1 g, 92% Yield). MS m/z 290.0 [M+H]⁺.

### Intermediate 5c: 2-(4-Cyclopropyl-2-(methoxymethoxy)phenyl)-4,4,5,5-tetramethyl-1,3,2-dioxaborolane

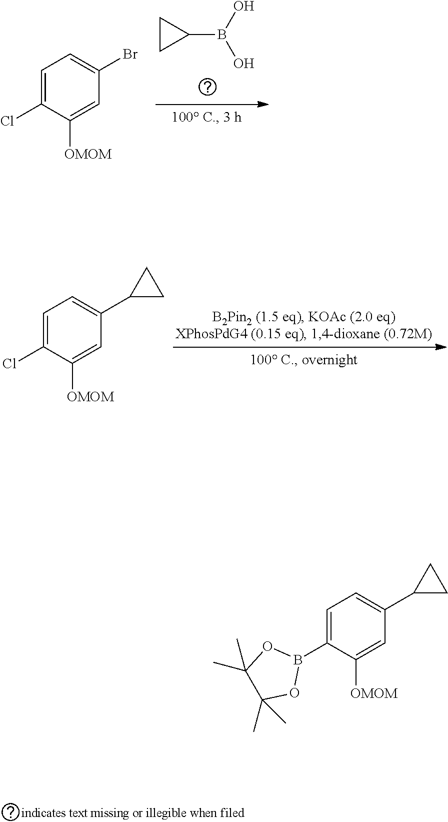

### Step 1. 1-Chloro-4-cyclopropyl-2-(methoxymethoxy)benzene

A mixture of 4-bromo-1-chloro-2-(methoxymethoxy)benzene (prepared according to the procedure of Intermediate 5a, step 1, 1.5 g, 6.0 mmol), cyclopropylboronic acid (670 mg, 7.8 mmol), potassium phosphate tribasic (4.5 g, 21 mmol), tricyclohexylphosphine (170 mg, 0.60 mmol) and palladium(ii) acetate (67 mg, 0.30 mmol) in water (2 mL) and toluene (40 mL) was stirred at 100° C. for 3 hours under N₂. After cooling to rt. the mixture was washed with water and brine, dried over Na₂SO₄ and concentrated to give a residue that was purified by flash chromatography on silica gel (EtOAC in PE=0%) to afford the product. 1-chloro-4-cyclopropyl-2-(methoxymethoxy)benzene (780 mg, 61% Yield).

### Step 2. 2-(4-Cyclopropyl-2-(methoxymethoxy)phenyl)-4,4,5,5-tetramethyl-1,3,2-dioxaborolane

A solution of 1-chloro-4-cyclopropyl-2-(methoxymethoxy)benzene (780 mg, 3.7 mmol), bis(pinacolato)diboron (1.4 g, 5.5 mmol), potassium acetate (720 mg, 7.3 mmol), XPhos Pd G4 (473 mg, 0.54 mmol) in 1,4-dioxane (5 mL) was stirred at 100° C. for 16 hours under N₂. After reaction, the mixture was poured into H₂O, extracted with DCM. The combined organic phase was dried over Na₂SO₄ and concentrated to give the crude product 2-(4-cyclopropyl-2-(methoxymethoxy)phenyl)-4,4,5,5-tetramethyl-1,3,2-dioxaborolane (1.3 g) which was used in the next step directly. ¹H NMR (400 MHz, DMSO-d6) δ: 7.43 (d, J=7.6 Hz, 1H), 6.76 (s, 1H), 6.68 (d, J=8.8 Hz, 1H), 5.14 (s, 2H), 3.41 (s, 3H), 1.93-1.82 (m, 1H), 1.27 (s, 12H), 1.00-0. 89 (m, 2H), 0.73-0.59 (m, 2H).

### Intermediate 5d: 2-(4-Chloro-2-fluoro-6-(methoxymethoxy)phenyl)-4,4,5,5-tetramethyl-1,3,2-dioxaborolane

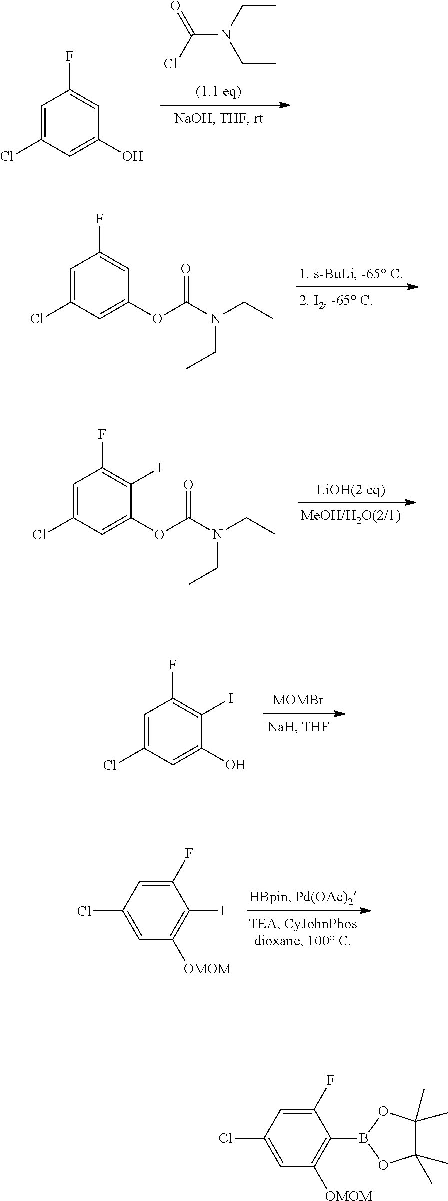

### Step 1. 3-Chloro-5-fluorophenyl diethylcarbamate

To a solution of 3-chloro-5-fluorophenol (1.46 g, 10 mmol) in doxane (15 mL) was added diethylcarbamic chloride (1.36 g, 10 mmol) and NaOH (800 mg, 20 mmol). The mixture was stirred at rt for 16 h, monitored by LCMS. Water was added, and the mixture was extracted with EA. The organic layers were washed by H₂O and brine, then dried and concentrated under vacuum to give residue which was purified by SGC (PE/EtOAc=10:1) to give 3-chloro-5-fluorophenyl diethylcarbamate (2.2 g, 8.9 mmol, 89.8% Yield) as a pale yellow oil.

### Step 2. 5-Chloro-3-fluoro-2-iodophenyl diethylcarbamate

To a solution of 3-chloro-5-fluorophenyl diethylcarbamate (2.2 g, 8.9 mmol) in THF (25 ml) was added s-BuLi (5.4 mL, 13.4 mmol, 2.5 mol/L) at −78° C. The mixture was stirred at −78° C. for 1.5 h and then a solution of I₂ (2.50 g, 1.1 eq) in THF (10 mL) was added in. This mixture was stirred at −78° C. for another 0.5 h. After that, the reaction was quenched with HCl (1 M), and warmed to room temperature. The mixture was diluted with aqueous Na₂S₂O₃ solution, extracted with EtOAc. The combined organic was dried over anhydrous Na₂SO₄ and concentrated under reduced pressure to give a residue which was purified by SGC (hexane/EtOAc=20:1) to get 5-chloro-3-fluoro-2-iodophenyl diethylcarbamate (2.3 g, 6.2 mmol, 69.1% Yield) as colorless oil. MS m/z 371.8 [M+H]⁺.

### Step 3. 5-Chloro-3-fluoro-2-iodophenol

To a solution of 5-chloro-3-fluoro-2-iodophenyl diethylcarbamate (2.3 g, 6.2 mmol) in MeOH:H₂O=2:1 (24 mL) was added LiOH·H₂O (525 mg, 12.5 mmol). The mixture was stirred at 60° C. for 16 h, monitored by LCMS. After that, 1M HCl was added to pH=7 and the mixture was extracted with EA. The organic layers were washed by H₂O and brine, then dried and concentrated under vacuum to give residue which was purified by column chromatography (PE/EtOAc=10:1) to give 5-chloro-3-fluoro-2-iodophenol (1.5 g, 5.5 mmol, 89.3% Yield) as colorless oil. MS m/z 270.9 [M−H]⁻.

### Step 4. 5-Chloro-1-fluoro-2-iodo-3-(methoxymethoxy)benzene

To a solution of 5-chloro-3-fluoro-2-iodophenol (540 mg, 2 mmol) in THE (6 mL) was added NaH (72 mg, 3 mmol) at 0° C. The reaction mixture was stirred at rt for 1 h, MOMBr (375 mg, 3 mmol) was added, stirred at rt for 0.5 h. After that, water was added to quench the reaction. The mixture was extracted with EA. The organic layers was washed by H₂O and brine, then dried and concentrated under vacuum to give residue which was purified by SGC (PE/EtOAc=10:1) to give 5-chloro-1-fluoro-2-iodo-3-(methoxymethoxy)benzene (550 mg, 1.73 mmol, 87% yield) as colorless oil.

### Step 5. 2-(4-Chloro-2-fluoro-6-(methoxymethoxy)phenyl)-4,4,5,5-tetramethyl-1,3,2-dioxaborolane

To a solution of 5-chloro-1-fluoro-2-iodo-3-(methoxymethoxy)benzene (550 mg, 1.73 mmol) in 1,4-dioxane (6 ml) was added Pd(OAc)₂ (55 mg, 0.24 mmol), 2-dicyclohexylphosphino)biphenyl (145 mg, 0.42 mmol), TEA (1.24 g, 12.1 mmol) and HBPin (1.1 g, 8.7 mmol), The reaction was heated to 80 degrees under nitrogen protection and stirred for 16 h, monitored by LCMS. After that, the mixture was concentrated to give a residue which was purification by SGC (PE/EtOAc=10:1) to give crude 2-(4-chloro-2-fluoro-6-(methoxymethoxy)phenyl)-4,4,5,5-tetramethyl-1,3,2-dioxaborolane (500 mg) as black oil. ¹H NMR (400 MHz, DMSO-d6) δ 6.99 (s, 2H), 5.22 (s, 2H), 3.38 (s, 3H), 1.30 (s, 12H).

The intermediates below were prepared according to the procedure of Intermediate 5d by substituting the appropriate starting materials, reagents and reaction conditions.

| Structure | Spectral Data |
|---|---|
| 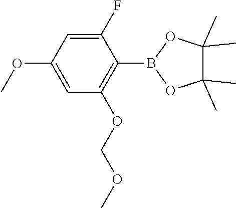 | ¹H NMR (400 MHz, DMSO-d6) δ 6.48-6.35 (m, 2H), 5.16 (s, 2H), 3.76 (s, 3H), 3.39 (s, 3H), 1.28 (s, 12H). |
| 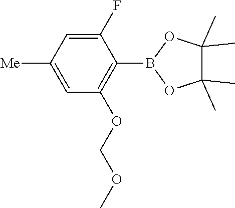 [SMILES: COCOc1cc(C)cc(F)c1B1OC(C)(C)C(C)(C)O1] | ¹H NMR (400 MHz, DMSO-d6) δ 6.72 (s, 1H), 6.61 (d, J = 11.6 Hz, 1H), 5.16 (s, 2H), 3.39 (s, 3H), 2.24 (s, 3H), 1.29 (s, 12H) |
| 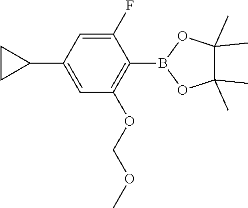 [SMILES: COCOc1cc(C2CC2)cc(F)c1B1OC(C)(C)C(C)(C)O1] | ¹H NMR (400 MHz, DMSO-d6) δ 6.62 (s, 1H), 6.44 (d, J = 9.6Hz, 1H), 5.16 (s, 2H), 3.38 (s, 3H), 1.94-1.87 (m, 1H), 1.28 (s, 12H), 0.99-0.91 (m, 2H), 0 .72- 0.65 (m, 2H). |
| 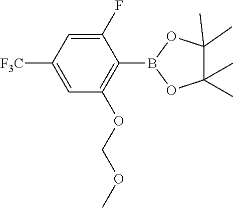 [SMILES: COCOc1cc(C(F)(F)F)cc(F)c1B1OC(C)(C)C(C)(C)O1] | ¹H NMR (400 MHz, DMSO-d6) δ 7.25-7.23 (m, 2H), 5.31 (s, 2H), 3.39 (s, 3H), 1.32 (s, 12H). |

### Intermediate 5e: 2-(2-(Benzyloxy)-4-(methoxy-d3)phenyl)-4,4,5,5-tetramethyl-1,3,2-dioxaborolane

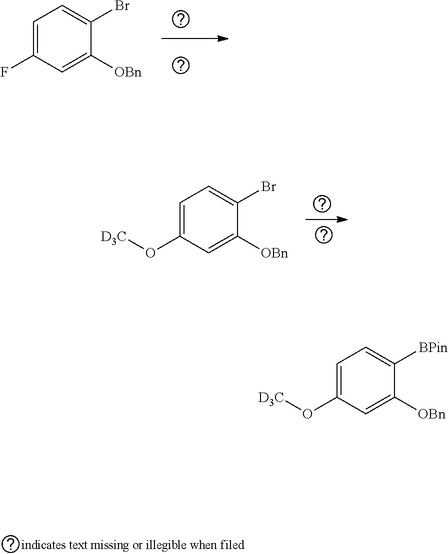

### Step 1: 2-(Benzyloxy)-1-bromo-4-(methoxy-d3) benzene

To a suspension of t-BuOK (3.40 g, 30 mmol, 3.9 eq.) in DMF (25 ml) was added MeOH-d4 (1.90 ml, 46 mmol, 6 eq.) dropwise under nitrogen at 0° C. The reaction mixture was allowed to stir at 0° C. for 15 mins. A solution of 2-(benzyloxy)-1-bromo-4-fluorobenzene (2.10 g, 7.60 mmol) in DMF (5 mL) was added into the reaction and the reaction mixture was heated to 80° C. for 20 h. The reaction was quenched with saturated NH₄Cl aqueous solution. Then reaction mixture was extracted with EA and washed with brine and dried over MgSO₄ and concentrated to dryness. The resulting oily residue was purified by chromatography on SiO₂ (EtOAc. Hexane, 0 to 75%) to afford the desired product (2.20 g, 90%) as a colorless oil. MS m/z 297.2 [M+H]⁺.

### Step 2. 2-(2-(Benzyloxy)-4-(methoxy-d3)phenyl)-4,4,5,5-tetramethyl-1,3,2-dioxaborolane

2-(Benzyloxy)-1-bromo-4-(methoxy-d3)benzene (1.00 g, 3.41 mmol), B₂Pin₂ (1.30 g, 5.12 mmol, 1.5 eq.), KOAc (1.00 g, 10.2 mmol, 3 eq.) and Pd(dppf)Cl₂ (0.26 g, 0.34 mmol, 0.1 eq.) was added in a vial and evacuated and backfilled with Ar before diluted with dioxane (17 mL). And the reaction was sparged with Ar for 5 min then was heated to 90° C. for 3 h. The reaction was cooled to rt and diluted with EtOAc. The organic phase was washed with water, brine and dried over Mg₂SO₄, filtered and concentrated. Purification by chromatography on SiO₂ (EtOAc. Hexane, 0 to 40%) gave a pale yellow oil (759 mg, 65%). MS m/z 344.1 [M+H]⁺.

### Intermediate 5f: 2-(2-(Benzyloxy)-6-fluoro-4-(methoxy-d3)phenyl)-4,4,5,5-tetramethyl-1,3,2-dioxaborolane

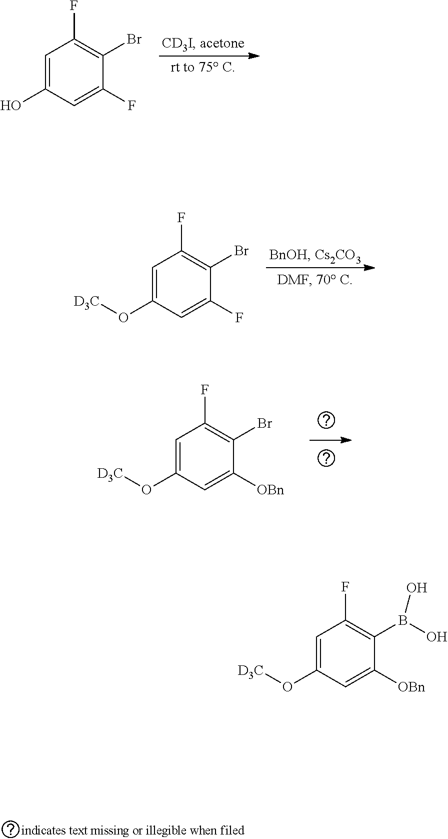 [SMILES: Oc1cc(F)c(Br)c(F)c1 ; [2H]C([2H])([2H])Oc1cc(F)c(Br)c(F)c1 ; [2H]C([2H])([2H])Oc1cc(F)c(Br)c(OCc2ccccc2)c1 ; [2H]C([2H])([2H])Oc1cc(F)c(B(O)O)c(OCc2ccccc2)c1]

### Step 1: 2-Bromo-1,3-difluoro-5-(trideuteriomethoxy)benzene

To a 250-mL round-bottom flask equipped with a magnetic stir bar under argon atmosphere was added 4-bromo-3,5-difluoro-phenol (4.97 g, 23.8 mmol) and acetone (120 mL, 5 L/mol). Potassium carbonate (6.8 g, 49 mmol, 2.1 equiv) was added at room temperature and the suspension was stirred vigorously. Iodomethane-d₃ (1.9 mL, 29.8 mmol, 1.25 equiv) was added slowly via syringe, and the reaction mixture was heated to 75° C. for 3 h. The mixture was allowed to cool to room temperature and filtered. The filtrate was concentrated under reduced pressure (40° C., 100 mmHg) and the resulting oil was taken up in diethyl ether. The organic layer was washed with saturated aqueous Na₂S₂O₃ and brine, dried over sodium sulfate, filtered, and concentrated to afford the crude product. Purification by column chromatography (silica, gradient 0% to 50% EtOAc in hexanes) afforded 2-bromo-1,3-difluoro-5-(trideuteriomethoxy)benzene (4.17 g, 78%) as a clear, colorless oil. ¹H NMR (400 MHz, DMSO-d6) δ 6.95 (d, J=9.0 Hz, 2H).

### Step 2. 1-Benzyloxy-2-bromo-3-fluoro-5-(trideuteriomethoxy)benzene

To a 120-mL test-tube equipped with a magnetic stir bar and a Teflon-lined cap was added 2-bromo-1,3-difluoro-5-(trideuteriomethoxy)benzene (2.55 g, 11.3 mmol) and DMF (22 mL, 2 L/mol). Cesium carbonate (7.2 g, 22 mmol, 2.0 equiv) was added and the mixture was stirred to even suspension. Benzyl alcohol (2.3 mL, 2.4 g, 22 mmol, 2.0 equiv) was added and the mixture was heated to 70° C. After 16 h the reaction had reached about 80% conversion as judged by LC-MS, so an additional 2 equivalents of cesium carbonate and benzyl alcohol were added. After an additional 8 h, the reaction was deemed complete by LC-MS. Cesium carbonate was removed by filtration through a plastic frit, and the filtrate was loaded directly onto silica. Chromatography (silica, gradient 0% to 40% dichloromethane in hexanes) afforded 1-benzyloxy-2-bromo-3-fluoro-5-(trideuteriomethoxy)benzene (2.44 g, 69%) as a clear, colorless oil. ¹H NMR (400 MHz, CDCl₃) δ 7.56-7.27 (m, 5H), 6.46-6.23 (m, 2H), 5.12 (s, 2H). ¹⁹F NMR (376 MHz, CDCl₃) δ −104.37 (d, J=8.3 Hz).

### Step 3: [2-Benzyloxy-6-fluoro-4-(trideuteriomethoxy)phenyl]boronic acid

To an oven-dried 100-mL Schlenk flask equipped with a magnetic stir bar under argon atmosphere was added 1-benzyloxy-2-bromo-3-fluoro-5-(trideuteriomethoxy)benzene (3.96 g, 12.6 mmol) and dry tetrahydrofuran (42 mL, 3.33 L/mol). The resulting solution was cooled in a dry ice/isopropanol bath to an internal temperature of −70° C. n-Butyllithium (5.6 mL, 2.7 M, 15.12 mmol, 1.20 equiv) was added dropwise, ensuring internal temperature did not rise above −65° C. Once the addition was complete, the mixture was stirred for an additional 20 minutes at −78° C. Tri-isopropyl borate (8.72 mL, 7.11 g, 37.8 mmol, 3.00 equiv) was added dropwise, again ensuring the internal temperature did not rise above −65° C. Once the addition was complete, the reaction mixture was stirred for 2 hours at −78° C. and then allowed to warm to room temperature overnight. A large amount of white precipitate began to form as the reaction mixture warmed. After 20 h, the reaction mixture was cooled to 0° C. in an ice-water bath and quenched by the slow addition of 1 M aqueous HCl (20 mL). The heterogeneous mixture was then warmed to room temperature and stirred for 1 h. The layers were then separated, and the aqueous layer was extracted 3 times with ethyl acetate. Combined organic layers were washed with brine and dried over sodium sulfate, filtered, and concentrated on the rotavap (40° C., 30 mmHg) to afford the crude product as a tan solid. Purification by column chromatography (0% to 50% EtOAc in hexanes) afforded [2-benzyloxy-6-fluoro-4-(trideuteriomethoxy)phenyl]boronic acid (1.75 g, 6.27 mmol, 50%) as an off-white solid that is best used immediately as it slowly decomposes in ambient conditions. MS m/z=278.1 [M−H]⁻; ¹H NMR (400 MHz, DMSO-d₆) δ 8.13 (br s, 2H), 7.62-7.21 (m, 5H), 6.55-6.24 (m, 2H), 5.08 (s, 2H). ¹⁹F NMR (376 MHz, DMSO-d₆) δ −103.94 (d, J=10.5 Hz).

### Intermediate 5g: 2-(2-(Benzyloxy)-6-fluoro-4-(methoxy-d3)phenyl)-4,4,5,5-tetramethyl-1,3,2-dioxaborolane

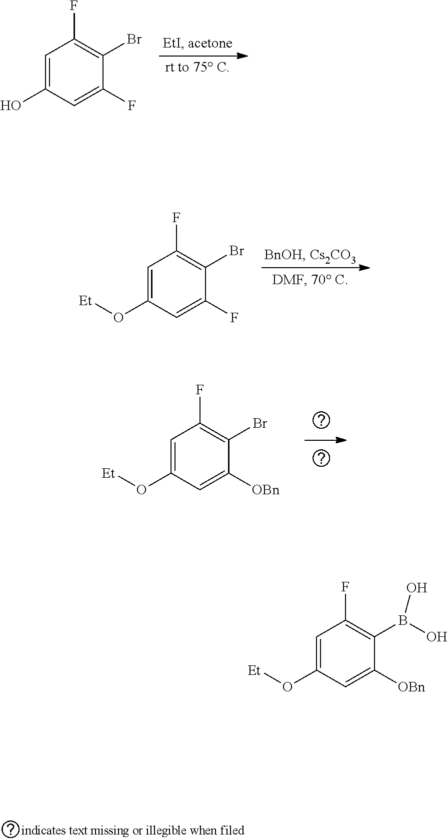 [SMILES: Oc1cc(F)c(Br)c(F)c1 ; CCOc1cc(F)c(Br)c(F)c1 ; CCOc1cc(F)c(Br)c(OCc2ccccc2)c1 ; CCOc1cc(F)c(B(O)O)c(OCc2ccccc2)c1]

The title compound was prepared in analogous manner according to the procedure of Intermediate 5g, using iodoethane in place of Iodomethane-d₃ in step 1. ¹H NMR (400 MHz, DMSO-d6) δ 8.11 (s, 2H), 7.47-7.28 (m, 5H), 6.39 (d, J=1.5 Hz, 1H), 6.30 (dd, J=10.5, 1.8 Hz, 1H), 5.07 (s, 2H), 4.01 (q, J=7.0 Hz, 2H), 1.29 (t, J=7.0 Hz, 3H).

### Intermediate 5 h: 2-(4-Chloro-2-(methoxymethoxy)-6-methylphenyl)-4,4,5,5-tetramethyl-1,3,2-dioxaborolane

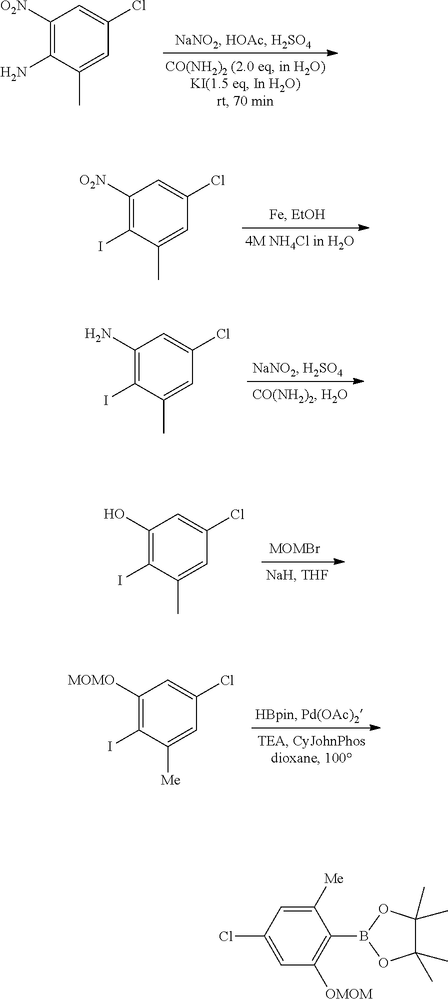

### Step 1. 5-Chloro-2-iodo-1-methyl-3-nitrobenzene

To a solution of 4-chloro-2-methyl-6-nitro-aniline (2.5 g, 13 mmol) in acetic acid (20 mL) was added a solution of sodium nitrite (1.46 g, 21.2 mmol) in H₂SO₄ (7.5 mL) dropwise. The mixture was allowed to stir at room temperature for 0.5 h. TLC (PE:EA=10:1) showed the reaction was complete. Water (40 mL) and urea (1.46 g, 24.3 mmol) were added in and the mixture was stirred at room temperature for 10 min. A solution of potassium iodide (3.2 g, 19 mmol) in H₂O (20 mL) was added dropwise and the mixture was stirred at room temperature for 30 minutes and filtered. The filter cake was washed with water and dried in vacuum to get 5-chloro-2-iodo-1-methyl-3-nitrobenzene (4 g, 13.4 mmol, 98% Yield) as a brown solid.

### Step 2. 5-Chloro-2-iodo-3-methylaniline

A stirred solution of ammonium chloride (2.15 g, 40.3 mmol) in water (10 mL) was added into a solution of 5-chloro-2-iodo-1-methyl-3-nitro-benzene (4 g, 13.4 mmol) in ethanol (30 mL). The mixture was heated to 50° C. and Iron (3.76 g, 67.2 mmol) was added at one portion. The mixture was allowed to stir at reflux for 30 min and TLC (PE:EA=10:1) showed the reaction was complete. The precipitate was filtered out. Ethanol was removed by evaporation and the aqueous solution was extracted with EtOAc. The organic layer was dried, filtered, and concentrated to get 5-chloro-2-iodo-3-methylaniline (3 g, 83.4% Yield) as a brown solid.

### Step 3. 5-Chloro-2-iodo-3-methylphenol

To a solution of 5-chloro-2-iodo-3-methyl-aniline (2 g, 7.5 mmol) in water (50 mL) was added H₂SO₄ (0.5 M, 50 mL). The solution was heated to 80° C. until all solid dissolved. Then the reaction was cooled to 0° C. and sodium nitrite (774 mg, 11.2 mmol) was added in small portions. After 2 h at this temperature, urea (450 mg, 7.5 mmol) was added at 0° C. The solution was allowed to warm up to room temperature and H₂SO₄ (0.5 M, 50 mL) was added. The reaction was refluxed for 30 min and cooled to room temperature. The solution was extracted with EtOAc and EtOH and the combined organic phases were dried over Na₂SO₄, purification by silica gel column (PE:EA=10:1), get 5-chloro-2-iodo-3-methylphenol (500 mg, 25% Yield) as red oil. MS m/z 266.9 [M−H]⁻.

### Step 4. 5-Chloro-2-iodo-1-(methoxymethoxy)-3-methylbenzene

To a solution of 5-chloro-2-iodo-3-methylphenol (500 mg, 1.9 mmol) in THE (5 mL) was added NaH (72 mg, 3 mmol) at 0° C. After that, the mixture was stirred at rt for 1 h. Then MOMBr (375 mg, 3 mmol) was added in, and the reaction mixture was stirred at rt for another 0.5 h. After that, water was added to quench the reaction. The mixture was extracted with EA. The organic layers were washed by H₂O and brine, then dried and concentrated under vacuum to give residue which was purified by SGC (PE/EtOAc=10:1) to give 5-chloro-2-iodo-1-(methoxymethoxy)-3-methylbenzene (450 mg, 1.44 mmol, 80% yield) as red oil.

### Step 5. 2-(4-Chloro-2-(methoxymethoxy)-6-methylphenyl)-4,4,5,5-tetramethyl-1,3,2-dioxaborolane

To a solution of 5-chloro-2-iodo-1-(methoxymethoxy)-3-methylbenzene (450 mg, 1.44 mmol) in 1,4-dioxane (6 ml) was added Pd(OAc)₂ (45 mg, 0.2 mmol), 2-(Dicyclohexylphosphino)biphenyl (121 mg, 0.35 mmol), TEA (1.01 g, 10 mmol) and HBPin (914 mg, 7.2 mmol). The mixture was stirred at 80 degrees under nitrogen protection for 16 h, monitored by LCMS. After that, the mixture was concentrated to give a residue which was purification by SGC (PE/EtOAc=10:1) to give 2-(4-chloro-2-(methoxymethoxy)-6-methylphenyl)-4,4,5,5-tetramethyl-1,3,2-dioxaborolane (169 mg, 0.54 mmol, 37.5% yield) as red oil.

### Intermediate 5i: 2-(2-(Methoxymethoxy)-6-methyl-4-(trifluoromethyl)phenyl)-4,4,5,5-tetramethyl-1,3,2-dioxaborolane

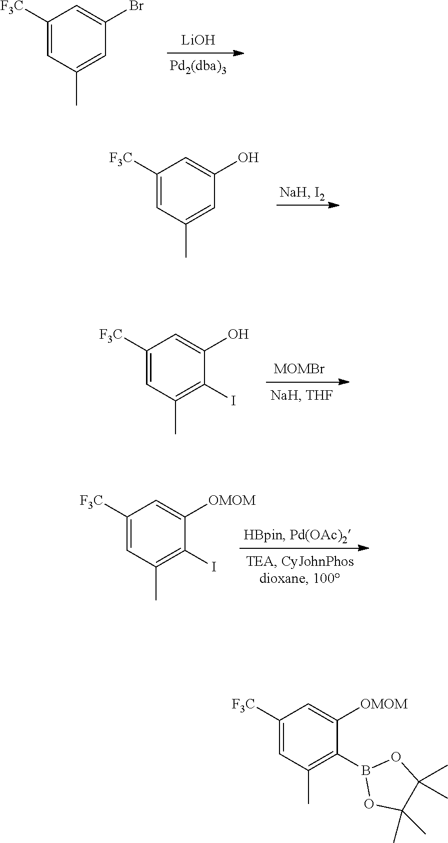

### Step 1. 3-Methyl-5-(trifluoromethyl)phenol

To a solution of 1-bromo-3-methyl-5-(trifluoromethyl)benzene (20 g, 83.7 mmol) in 1,4-Dioxane (200 ml) and H₂O (20 ml) were added LiOH·H₂O (4.0 g, 167.3 mmol), Pd₂(dba)₃ (3.8 g, 4.2 mmol) and BippyPhos (1.7 g, 3.35 mmol). The reaction mixture was purged with Ar and stirred at 100° C. for 16 hours, monitored by TLC. After that, the mixture was cooled to RT, diluted with EtOAc (200 ml), washed with 1.5 M HCl and dried over Na₂SO₄ and concentrated. The residue was purified by silica gel column to give 3-methyl-5-(trifluoromethyl)phenol (13 g, 73.4 mmol, 88% Yield). MS m/z 175.0 [M−H]⁺.

### Step 2. 2-Iodo-3-methyl-5-(trifluoromethyl)phenol

To a solution of 3-methyl-5-(trifluoromethyl)phenol (13 g, 73.4 mmol) in toluene (390 ml) was added NaH (3.54 g, 147.6 mmol) at 0° C. The suspension was stirred at same temperature for 30 min. And then iodine (13.87 g, 55.1 mmol) was added slowly in portions and the mixture was stirred for 3 hours. After that, the mixture was diluted with water, acidified with 1 M HCl to pH 5 and extracted with EtOAc. The organic layer was washed with brine, dried over Na₂SO₄ and concentrated. The residue was purified by silica gel column to give 2-iodo-3-methyl-5-(trifluoromethyl)phenol (6.6 g, 22.0 mmol, 30% Yield). MS m/z 300.9 [M−H]⁺.

### Step 3. 2-Iodo-1-(methoxymethoxy)-3-methyl-5-(trifluoromethyl)benzene

To a solution of 2-iodo-3-methyl-5-(trifluoromethyl)phenol (5.9 g, 20 mmol) in THE (60 ml) was added NaH (936 mg, 39 mmol) at 0° C. The mixture was stirred at same temperature for 10 min, and then MOMBr (2.88 g, 23 mmol) was added in. This reaction solution was stirred at room temperature for 2 hours. After that, solvent was removed and the residue was purified by silica gel column to give 2-iodo-1-(methoxymethoxy)-3-methyl-5-(trifluoromethyl)benzene (4 g, 11.6 mmol, 59% Yield). ¹H NMR (400 MHz, CDCl₃) δ: 7.17 (s, 1H), 7.09 (s, 1H), 5.28 (s, 2H), 3.52 (s, 3H), 2.53 (s, 3H).

### Step 4. 2-(2-(Methoxymethoxy)-6-methyl-4-(trifluoromethyl)phenyl)-4,4,5,5-tetramethyl-1,3,2-dioxaborolane

To a solution of 2-iodo-1-(methoxymethoxy)-3-methyl-5-(trifluoromethyl)benzene (4 g, 11.6 mmol) in anhydrous 1, 4-dioxane (24 ml) were added pinacolborane (7.4 g, 58 mmol), biphenyl-2-yl-dicyclohexylphosphane (975 mg, 2.78 mmol), Pd(OAc)₂ (339 mg, 1.51 mmol) and TEA (8.2 g, 81.2 mmol). The mixture was purged with Ar and stirred at 80° C. for 16 hours. After that, the mixture was diluted with EtOAc and washed with saturated NH₄Cl, water and brine. The organic layer was dried over NaSO₄ and concentrated. The residue was purified by silica gel column to give 2-(2-(methoxymethoxy)-6-methyl-4-(trifluoromethyl)phenyl)-4,4,5,5-tetramethyl-1,3,2-dioxaborolane (3 g, 8.7 mmol, 74.7% Yield). ¹H NMR (400 MHz, CDCl₃) δ 7.05 (s, 2H), 5.16 (s, 2H), 3.47 (s, 3H), 2.39 (s, 3H), 1.39 (s, 12H).

### Intermediate 6a: 2-[2-(Difluoromethyl)-4-(trifluoromethyl)phenyl]-4,4,5,5-tetramethyl-1,3,2-dioxaborolane

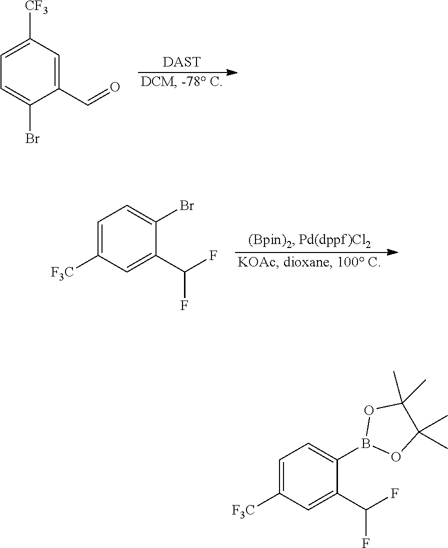

### Step 1. 1-Bromo-2-(difluoromethyl)-4-(trifluoromethyl)benzene

DAST (10 eq., 39.5 mmol) was added dropwise to a −78° C. solution of 2-bromo-5-(trifluoromethyl)benzaldehyde (1 g, 3.9 mmol) in DCM (5 mL, 78.0 mmol). The reaction was stirred for 15 min and then allowed to warm to room temperature. After 4 hours, TLC showed incomplete conversion, so the mixture was cooled again to −78° C. and addition DAST (1 eq., 3.9 mmol) was added. The mixture was allowed to warm to rt. And was stirred overnight. The mixture was poured in ice and dilute NH₄OH and extracted 2× with DCM. The combined organic extracts were washed with brine and dried over sodium sulfate. Solvent evaporated in vacuum to give a residue which was purified with a short plug of silica to give the crude product 1-bromo-2-(difluoromethyl)-4-(trifluoromethyl)benzene (350 mg, 1.2 mmol, 32.2% Yield) as colorless oil.

### Step 2. 2-[2-(Difluoromethyl)-4-(trifluoromethyl)phenyl]-4,4,5,5-tetramethyl-1,3,2-dioxaborolane

To a solution of 1-bromo-2-(difluoromethyl)-4-(trifluoromethyl)benzene (100 mg, 0.36 mmol) in 1,4-dioxane (2 mL) was added bis(pinacolato)diboron (1.5 eq., 0.5 mmol), potassium acetate (2 eq., 0.72 mmol) and [1,1′-bis(diphenylphosphino)ferrocene]dichloropalladium(ii) (0.15 eq., 0.05 mmol). The mixture was stirred for 16 h at 100° C. for 16 hours under N₂ atmosphere, monitored by TLC and LCMS. After the reaction, the solution was removed under vacuum and the residue was purified by column chromatography (10-15% EA in PE) to afford 2-[2-(difluoromethyl)-4-(trifluoromethyl)phenyl]-4,4,5,5-tetramethyl-1,3,2-dioxaborolane (30 mg, 0.09 mmol, 25.6% Yield) as colorless oil. ¹H NMR (400 MHz, DMSO-d6) δ 8.00 (d, J=7.6 Hz, 1H), 7.94 (d, J=8.4 Hz, 1H), 7.92 (s, 1H), 7.38 (t, J=55.6 Hz, 1H), 1.34 (s, 12H).

### Intermediate 6b: 2-(4-Chloro-2-(difluoromethyl)phenyl)-4,4,5,5-tetramethyl-1,3,2-dioxaborolane

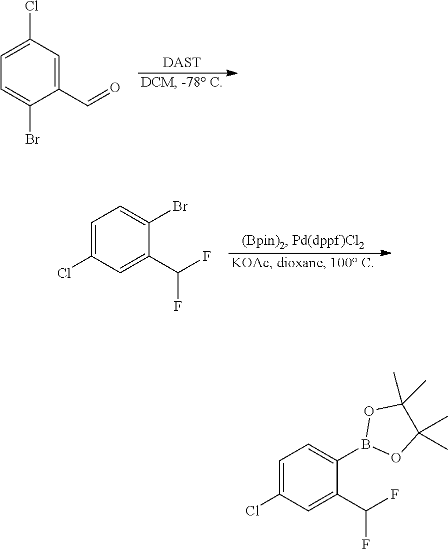

The title compound was prepared in analogous manner according to the procedure of Intermediate 6a, using 2-bromo-5-chlorobenzaldehyde in place of 2-bromo-5-(trifluoromethyl)benzaldehyde in step 1. ¹H NMR (400 MHz, DMSO-d6) δ 7.79 (d, J=2.0 Hz, 1H), 7.68-7.63 (m, 2H), 7.32 (t, J=55.6 Hz, 1H), 1.32 (s, 12H).

### Intermediate 7a: 2-(2-(Difluoromethoxy)-4-(trifluoromethyl)phenyl)-4,4,5,5-tetramethyl-1,3,2-dioxaborolane

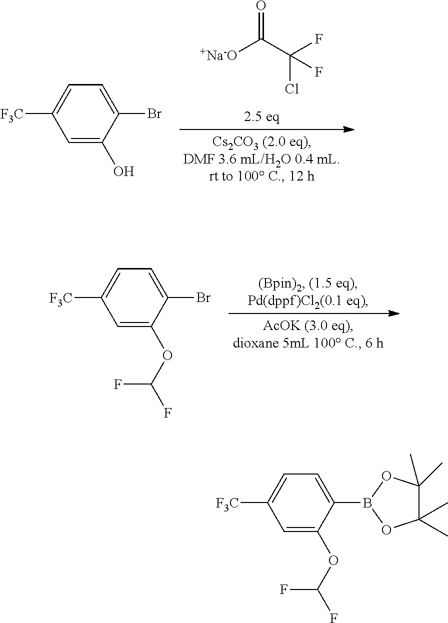

### Step 1. 1-Bromo-2-(difluoromethoxy)-4(trifluoromethyl)benzene

A mixture of 2-bromo-5-(trifluoromethyl)phenol (5.00 g, 20.7 mmol, 1.0 eq.), sodium 2-chloro-2,2-difluoroacetae (7.07 g, 51.9 mmol, 2.50 eq.) and cesium carbonate (11.5 g, 41.5 mmol, 2.0 eq.) in water (20 mL) and N,N-dimethylformamide (80 mL) was heated at 100° C. for 12 hours. Upon completion, the reaction mixture was cooled to room temperature and diluted with EtOAc. The reaction mixture was washed with water and brine. The organic phase was dried over Na₂SO₄, filtered, and concentrated under reduced pressure. The crude residue was purified by silica gel column chromatography eluting with 0-50% EtOAc in hexanes to afford 1-bromo-2-difluoromethoxy)-4(trifluoromethyl)benzene (2.8 g, 47% yield) as a yellow solid. ¹H NMR (400 MHz, DMSO-d6) δ 8.01 (d, J=8.4 Hz, 1H), 7.69 (s, 1H), 7.46 (t, J=72.8 Hz, 1H), 7.5 (d, J=8.4 Hz, 1H).

### Step 2. 2-(2-(Difluoromethoxy)-4-(trifluoromethyl)phenyl)-4,4,5,5-tetramethyl-1,3,2-dioxaborolane

To a solution of 1-bromo-2-difluoromethoxy)-4(trifluoromethyl)benzene (2.80 g, 9.62 mmol) in 1,4-dioxane (30 mL) was added bis(pinacolato)diboron (36.6 g, 14.4 mmol, 1.50 eq.), [1,1′-bis(diphenylphosphino)ferrocene]dichloropalldium(II) (704 mg, 0.96 mmol, 0.1 eq.), potassium acetate (2.82 g, 28.9 mmol, 3.0 eq.). The reaction mixture was heated at 100° C. for 16 hours under nitrogen. The reaction mixture was cooled to room temperature and filtered. The filtrate was diluted EA, washed with water and brine. The organic phase was dried over Na₂SO₄, filtered and concentrated under reduced pressure. The crude was purified by silica gel column chromatography eluting with hexane to afford 2-(2-(difluoromethoxy)-(trifluoromethyl)phenyl)-4,4,5,5-tetramethyl-1,3,2-dioxaborolane as a white solid (1.70 g, 52% yield). ¹H NMR (400 MHz, DMSO-d6) δ 7.87 (d, J=7.6 Hz, 1H), 7.66 (d, J=7.6 Hz, 1H), 7.50 (s, 1H), 7.24 (t, J=74.0 Hz, 1H), 1.31 (s, 12H).

The intermediates below were prepared according to the procedure of Intermediate 7a by substituting the appropriate starting materials, reagents and reaction conditions.

| Structure | Spectral Data |
|---|---|
| 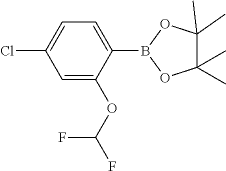 [SMILES: CC1(C)OB(c2ccc(Cl)cc2OC(F)F)OC1(C)C] | MS m/z 304.5 [M + H]⁺. ¹H NMR (400 MHz, DMSO-d6) δ 7.68 (d, J = 8.4 Hz, 1H), 7.38 (d, J = 7.6 Hz, 1H), 7.30 (s, 1H), 7.16 (t, J = 74.4 Hz, 1H), 1.30 (s, 12H). |
| 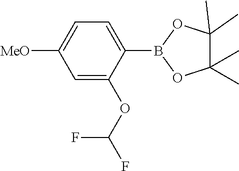 [SMILES: COc1ccc(B2OC(C)(C)C(C)(C)O2)c(OC(F)F)c1] | ¹H NMR (400 MHz, DMSO-d6) δ 7.60 (d, J = 8.4 Hz, 1H), 7.05 (t, J = 70.4 Hz, 1H), 6.86 (dd, J = 8.6, 2.0 Hz, 1H), 6.70 (d, J = 2.0 Hz, 1H), 3.80 (s, 3H), 1.27 (s, 12H). |
| 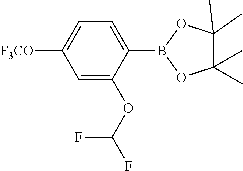 [SMILES: CC1(C)OB(c2ccc(OC(F)(F)F)cc2OC(F)F)OC1(C)C] | ¹H NMR (400 MHz, CDCl₃) δ 7.79 (d, J = 8.3 Hz, 1H), 7.11 (d, J = 8.2 Hz, 1H), 7.02 (s, 1H), 6.53 (t, J = 74.7 Hz, 1H), 1.26 (s, 12H). |

### Intermediate 7b: 2-(2-(Difluoromethoxy)-6-fluoro-4-methylphenyl)-4,4,5,5-tetramethyl-1,3,2-dioxaborolane

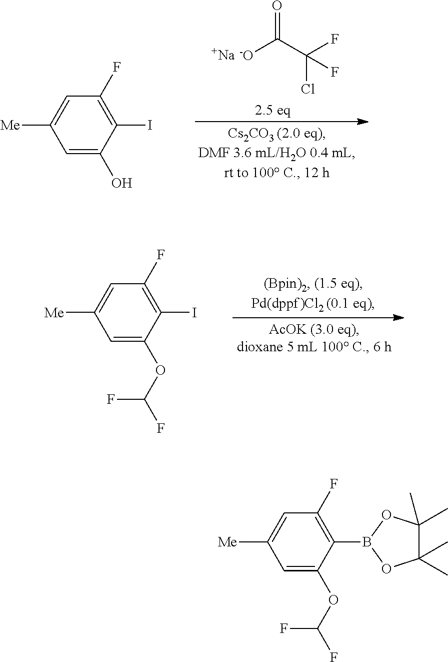

The starting material, 3-fluoro-2-iodo-5-methylphenol, was prepared in analogous manner according to the procedure of Intermediate 5d, step 1 to 3, using 3-fluoro-5-methylphenol in place of 3-chloro-5-fluorophenol in step 1.

The title compound was prepared in analogous manner according to the procedure of Intermediate 7a, using 3-fluoro-2-iodo-5-methylphenol in place of 2-bromo-5-(trifluoromethyl)phenol in step 1. ¹H NMR (400 MHz, DMSO-d6) δ 6.92 (d, J=10.0 Hz, 1H), 6.87 (s, 1H), 6.13 (t, J=74.4 Hz, 1H), 2.34 (s, 3H), 1.29 (s, 12H).

The intermediates below were prepared according to the procedure of Intermediate 7b by substituting the appropriate starting materials, reagents and reaction conditions.

| Structure | Spectral Data |
|---|---|
| 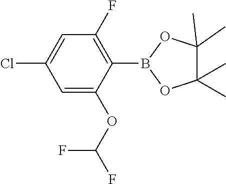 [SMILES: CC1(C)OB(c2c(F)cc(Cl)cc2OC(F)F)OC1(C)C] | ¹H NMR (400 MHz, DMSO-d6) δ 7.36 (dd, J = 8.8 Hz, 1.6 Hz, 1H), 7.27 (t, J = 73.6 Hz, 1H), 7.20 (s, 1H), 1.31 (s, 12H). |
| 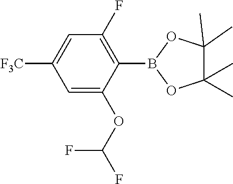 [SMILES: CC1(C)OB(c2c(F)cc(C(F)(F)F)cc2OC(F)F)OC1(C)C] | ¹H NMR (400 MHz, DMSO-d6) δ 7.60 (d, J = 8.4 Hz, 1H), 7.37 (t, 1H), 7.42 (s, 1H), 7.37 (t, J = 73.6 Hz, 1H), 1.32 (s, 12H). |

### Intermediate 7c: 2-(2,4-Bis(difluoromethoxy)phenyl)-4,4,5,5-tetramethyl-1,3,2-dioxaborolane

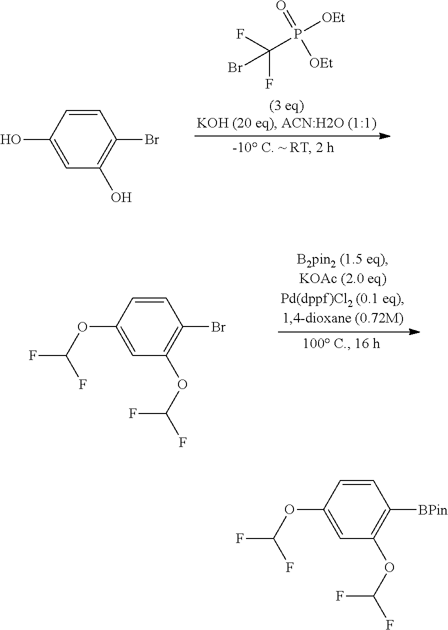

### Step 1. 1-Bromo-2,4-bis(difluoromethoxy)benzene

To a solution of 4-bromobenzene-1,3-diol (10.0 g, 53.4 mmol, 1.0 eq.) in ACN/H₂O (1:1, 100 mL) was slowly added KOH (60.0 g, 1.07 mol, 20 eq.) at 0° C. The mixture was stirred for 10 min and cooled to −10° C. Diethyl (bromodifluoromethyl) phosphonate (42.70 g, 160.2 mmol, 3.0 eq.) was slowly added and the mixture was stirred for 10 min and warmed to room temperature and stirred for 2 h. The reaction mixture was diluted with water (100 mL) and extracted with EtOAc (100 mL×3). The combined organic layers were washed with brine, dried over anhydrous Na₂SO₄ and concentrated. The residue was purified by flash chromatography on silica gel (PE:EA=0%˜5%) to give 1-bromo-2,4-bis(difluoromethoxy)benzene (3.30 g, 21.3% Yield) as yellow oil. ¹H NMR (400 MHz, DMSO-d6) δ: 7.84 (d, J=8.8 Hz, 1H), 7.38 (t, J=72.0 Hz, 1H), 7.35 (t, J=76.0 Hz, 1H), 7.26 (d, J=2.8 Hz, 1H), 7.11 (dd, J=8.8, 2.8 Hz, 1H).

### Step 2. 2-(2,4-Bis(difluoromethoxy)phenyl)-4,4,5,5-tetramethyl-1,3,2-dioxaborolane

To a solution of 1-bromo-2,4-bis(difluoromethoxy)benzene (3.30 g, 11.4 mmol, 1.0 eq.) in 1,4-dioxane was added KOAc (2.23 g, 22.8 mmol, 2.0 eq.), Pd(dppf)Cl₂ (0.830 g, 1.10 mmol, 0.1 eq.) and bis(pinacolato)diboron (4.34 g, 17.1 mmol, 1.5 eq.). The reaction mixture was stirred for 16 h at 100° C. under N₂. The mixture was filtered and concentrated. The residue was purified by flash chromatography on silica gel (PE:EA=0%˜10%) to obtain 2-(2,4-bis(difluoromethoxy) phenyl)-4,4,5,5-tetramethyl-1,3,2-dioxaborolane (2.0 g, 6.0 mmol, 52% Yield) as yellow oil. ¹H NMR (400 MHz, CD₃OD) δ 7.72 (d, J=8.0 Hz, 1H), 7.36 (t, J=73.2 Hz, 1H), 7.12 (t, J=74.4 Hz, 1H), 7.12-7.08 (m, 1H), 7.02-6.97 (m, 1H), 1.29 (s, 12H).

### Intermediate 8: N-Methyl-2-(4,4,5,5-tetramethyl-1,3,2-dioxaborolan-2-yl)-5-(trifluoromethyl)benzenesulfonamide

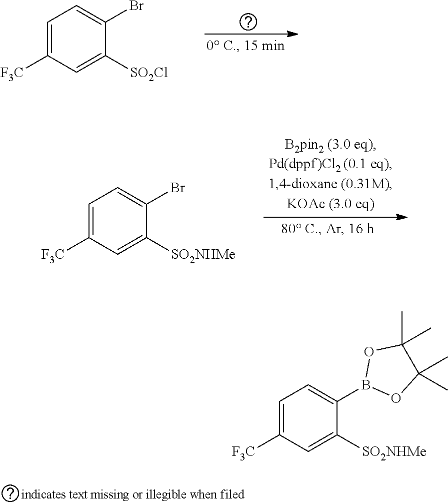

### Step 1. 2-Bromo-N-methyl-5-(trifluoromethyl)benzenesulfonamide

To a solution of 2-bromo-5-(trifluoromethyl)benzenesulfonyl chloride (1.00 g, 3.09 mmol, 1.0 eq.) and triethylamine (942 mg, 9.27 mmol, 3.0 eq.) in DCM (10 mL) was added methylamine (392 mg, 27% in EtOH, 3.42 mmol, 1.1 eq.) dropwise at 0° C. The reaction mixture was stirred at room temperature for 15 min. Upon completion, the reaction mixture was diluted with DCM (80 mL) and washed with 1N HCl (50 mL), aqueous sat. NaHCO₃ (50 mL) and brine (50 mL). The organic layer was dried over Na₂SO₄, filtered and concentrated under reduced pressure. The residue was purified by silica gel column chromatography eluting with 0-20% EtOAc in hexanes to afford 2-bromo-N-methyl-5-(trifluoromethyl)benzenesulfonamide (930 mg, 2.92 mmol, 94.6% yield) as a white solid. ¹H NMR (400 Hz, DMSO-d6) δ: 8.17 (d, J=1.8 Hz, 1H), 8.14 (d, J=8.2 Hz, 1H), 7.97 (s, 1H), 7.95 (dd, J=8.4, 2.0 Hz, 1H), 2.54 (s, 3H).

### Step 2. N-Methyl-2-(4,4,5,5-tetramethyl-1,3,2-dioxaborolan-2-yl)-5-(trifluoromethyl) benzenesulfonamide

A mixture of 2-bromo-N-methyl-5-(trifluoromethyl) benzenesulfonamide (930 mg, 2.92 mmol, 1.0 eq.), bis(pinacolato)diboron (2.23 g, 8.78 mmol, 3.0 eq.), Pd(dppf)Cl₂—CH₂Cl₂ (239 mg, 0.292 mmol, 0.1 eq.) and potassium acetate (860 mg, 8.76 mmol, 3.0 eq.) in anhydrous 1,4-dioxane (9 mL) was stirred at 80° C. overnight under N₂ for 16 h. Upon completion, the reaction mixture was cooled to room temperature, filtered and rinsed with EtOAc (40 mL). The filtrate was concentrated under reduced pressure and the crude residue was purified by silica gel column chromatography eluting with 0-10% EtOAc in hexane to afford N-methyl-2-(4,4,5,5-tetramethyl-1,3,2-dioxaborolan-2-yl)-5-(trifluoromethyl)benzenesulfonamide (192 mg, 0.526 mmol, 18.0% yield) as a brown solid. ¹H NMR (400 Hz, DMSO-d6) δ: 8.21 (s, 1H), 7.90 (d, J=7.6 Hz, 1H), 7.79 (d, J=7.6 Hz, 1H), 2.63 (d, J=5.2 Hz, 3H), 1.42 (s, 12H).

### Intermediate 9: 4,4,5,5-Tetramethyl-2-(2-(methylsulfonyl)-4-(trifluoromethyl)phenyl)-1,3,2-dioxaborolane

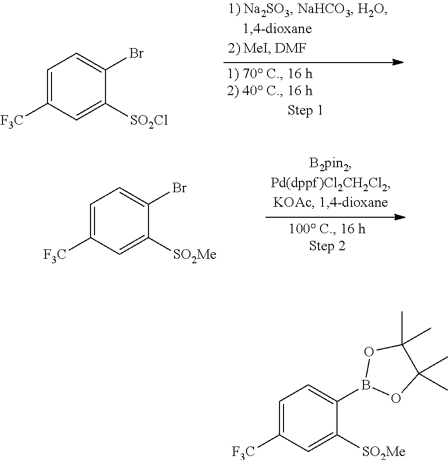 [SMILES: O=S(=O)(Cl)c1cc(C(F)(F)F)ccc1Br ; CS(=O)(=O)c1cc(C(F)(F)F)ccc1Br ; CC1(C)OB(c2ccc(C(F)(F)F)cc2S(C)(=O)=O)OC1(C)C]

### Step 1. 1-Bromo-2-(methylsulfonyl)-4-(trifluoromethyl)benzene

Sodium bicarbonate (779 mg, 9.27 mmol, 3.0 eq.) and sodium sulfite (779 mg, 6.18 mmol, 2.0 eq.) were heated in water (5 mL) at 70° C. for 5 min. A mixture of 2-bromo-5-(trifluoromethyl) benzenesulfonyl chloride (1.00 g, 3.09 mmol, 1.0 eq.) in 1,4-dioxane (10 mL) was added to the above mixture and stirred at 70° C. overnight. The solvents were removed in vacuo, then DMF (3 mL) and methyl iodide (483 mg, 3.43 mmol, 1.1 eq.) were added and reaction mixture was stirred at 40° C. overnight. Upon completion, the reaction mixture was cooled to room temperature and diluted with water (30 mL). The mixture was extracted with EtOAc (30 mL×3). The combined organic layers were dried over Na₂SO₄, filtered and concentrated. The residue was purified by silica gel column chromatography eluting with 0-30% EtOAc in hexanes to afford 1-bromo-2-(methylsulfonyl)-4-(trifluoromethyl) benzene as an orange oil (900 mg, 2.97 mmol, 96.1% yield). ¹H NMR (400 Hz, DMSO-d6) δ: 8.26 (d, J=2.0 Hz, 1H), 8.21 (d, J=8.2 Hz, 1H), 8.05 (dd, J=8.2, 2.2 Hz, 1H), 3.48 (s, 3H).

### Step 2. 4,4,5,5-Tetramethyl-2-(2-(methylsulfonyl)-4-(trifluoromethyl) phenyl)-1,3,2-dioxaborolane

A mixture of 1-bromo-2-(methylsulfonyl)-4-(trifluoromethyl) benzene (1.00 g, 3.30 mmol, 1.0 eq.), bis(pinacolato)diboron (2.51 g, 9.90 mmol, 3.0 eq.), Pd(dppf)Cl₂—CH₂Cl₂ (239 mg, 0.330 mmol, 0.1 eq.) and potassium acetate (970 mg, 9.90 mmol, 3.0 eq.) in anhydrous 1,4-dioxane (9 mL) was stirred at 100° C. overnight under N₂ for 16 h. Upon completion, the reaction mixture was cooled to room temperature, filtered and rinsed with EtOAc (40 mL). The filtrate was concentrated under reduced pressure and the crude residue was purified by silica gel column chromatography eluting with 0-10% EtOAc in hexanes to afford 4,4,5,5-tetramethyl-2-(2-(methylsulfonyl)-4-(trifluoromethyl) phenyl)-1,3,2-dioxaborolane (858 mg, 2.45 mmol, 74.2% yield) as a brown solid. ¹H NMR (400 Hz, DMSO-d6) δ: 8.21 (s, 1H), 8.12 (d, J=7.6 Hz, 1H), 7.89 (d, J=7.6 Hz, 1H), 3.36 (s, 3H), 1.35 (s, 12H).

### Intermediate 10: (E)-N,N-Dimethyl-N′-((2-(4,4,5,5-tetramethyl-1,3,2-dioxaborolan-2-yl)-5-(trifluoromethyl)phenyl)sulfonyl)formimidamide

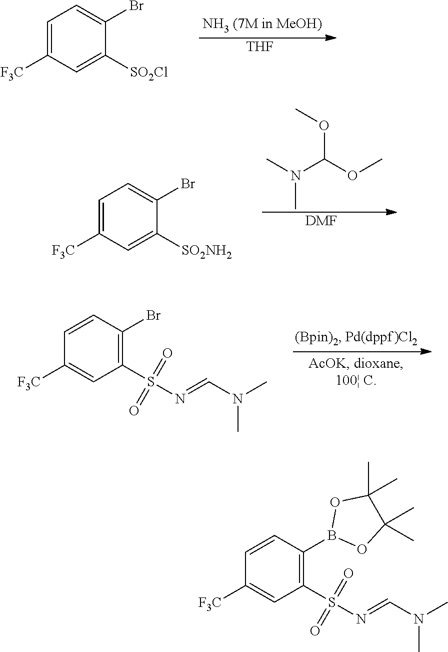

### Step 1. 2-Bromo-5-(trifluoromethyl)benzenesulfonamide

To ammonia (15 ml, 105 mmol, 7 M in methanol) was added a THF (70 mL) solution of 2-bromo-5-(trifluoromethyl)benzenesulfonyl chloride (7 g, 21.6 mmol). This reaction mixture was stirred at RT for 3 h, monitoring by LCMS. After that, solvent was removed and the residue was purification by SGC (PE:EA=3:1) to give 2-bromo-5-(trifluoromethyl)benzenesulfonamide (4.5 g, 15 mmol, 68% Yield) as colorless oil. MS m/z 301.8 [M+H]⁺.

### Step 2. (E)-N′-((2-Bromo-5-(trifluoromethyl)phenyl)sulfonyl)-N,N-dimethylformimidamide

To a solution of 2-bromo-5-(trifluoromethyl)benzenesulfonamide (4.5 g, 15 mmol) in DMF (50 mL) was added N,N-dimethylformamide dimethyl acetal (2.6 g, 22 mmol). This mixture was stirred at RT for 0.5 h, monitoring by LCMS. After that, the reaction mixture was diluted by water (100 mL) and extract by EtOAc (100 mL×3). The combined organic was dried over Na₂SO₄, concentrated and purification by silica gel column (PE:EA=3:1) to get (E)-N′-((2-bromo-5-(trifluoromethyl)phenyl)sulfonyl)-N,N-dimethylformimidamide (3.9 g, 73% Yield) as white solid. MS m/z 360.9 [M+H]⁺.

### Step 3. (E)-N,N-Dimethyl-N′-((2-(4,4,5,5-tetramethyl-1,3,2-dioxaborolan-2-yl)-5-(trifluoromethyl)phenyl)sulfonyl)formimidamide

A solution of (E)-N′-((2-bromo-5-(trifluoromethyl)phenyl)sulfonyl)-N,N-dimethylformimidamide (3.9 g, 11 mmol), bis(pinacolato)diboron (3.35 g, 13.2 mmol), potassium acetate (2200 mg, 22 mmol), and Pd(dppf)Cl₂ (800 mg, 1.1 mmol) in 1,4-dioxane (40 mL) was stirred at 90° C. under N₂ for 16 h, monitoring by LCMS. After reaction, solvent was removed and the residue was purified by silica gel column (PE:EA=3:1) to get (E)-N,N-dimethyl-N′-((2-(4,4,5,5-tetramethyl-1,3,2-dioxaborolan-2-yl)-5-(trifluoromethyl)phenyl)sulfonyl)formimidamide (300 mg, 9% Yield) as yellow oil. MS m/z 407.3 [M+H]⁺.

### Intermediate 11a: 2-(4-Chloro-2,6-difluorophenyl)-4,4,5,5-tetramethyl-1,3,2-dioxaborolane

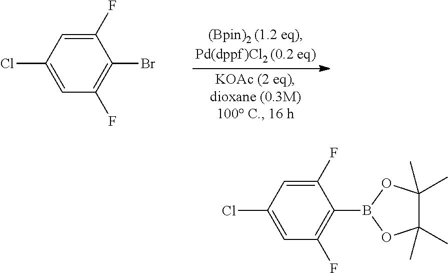 [SMILES: Fc1cc(Cl)cc(F)c1Br ; CC1(C)OB(c2c(F)cc(Cl)cc2F)OC1(C)C]

To a solution of 2-bromo-5-chloro-1,3-difluorobenzene (200 mg, 0.880 mmol, 1.0 eq.) in 1,4-dioxane (3 mL, 0.3 M) was added bis(pinacolato)diboron (246 mg, 0.970 mmol, 1.1 eq.), 1,1′-bis(diphenyphosphino)ferrocene (25 mg, 0.044 mmol, 0.05 eq.), Pd(dppf)Cl₂ (32 mg, 0.044 mmol, 0.05 eq.) and KOAc (259 mg, 2.64 mmol, 3.0 eq.). The reaction mixture was stirred for 8 h at 80° C. under N₂. The mixture was diluted with water (10 mL) and extracted with EtOAc (10 mL×2). The organic layer was washed with brine, dried over anhydrous Na₂SO₄ and evaporated in vacuo. The residue was purified by flash chromatography on silica gel (PE:EA=50:1 to 20:1) to obtain 2-(4-chloro-2,6-difluorophenyl)-4,4,5,5-tetramethyl-1,3,2-dioxaborolane (220 mg, 0.80 mmol, 91% yield) as a white solid. ¹H NMR (400 MHz, DMSO-d6) δ 7.64 (d, J=6.8 Hz, 2H), 1.23 (s, 12H).

### Intermediate 11b: 2-(4-Chloro-2-fluoro-6-methylphenyl)-4,4,5,5-tetramethyl-1,3,2-dioxaborolane

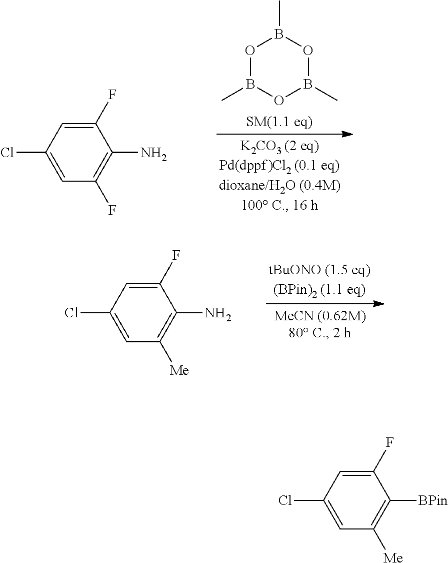

### Step 1. 4-Chloro-2-fluoro-6-methylaniline

To a solution of 2-bromo-4-chloro-6-fluoroaniline (5.00 g, 22.3 mmol, 1.0 eq.) in 1,4-dioxane/H₂O (5:1, 50 mL) were added 2,4,6-trimethyl-1,3,5,2,4,6-trioxatriborinane (3.07 g, 24.5 mmol, 1.1 eq.), K₂CO₃ (6.15 g, 44.6 mmol, 2.0 eq.) and Pd(dppf)Cl₂ (1.63 g, 2.2 mmol, 0.1 eq.). The reaction mixture was stirred for 16 h at 100° C. under N₂. The mixture was poured into water (100 mL) and extracted with DCM (100 mL×2). The organic layer was washed with brine, dried over anhydrous Na₂SO₄, and evaporated in vacuum. The residue was concentrated in vacuum to give 4-chloro-2-fluoro-6-methylaniline (1.50 g, crude) as yellow oil which was used directly to the next step without further purification. MS m/z 160.2 [M+H]⁺,

### Step 2. 2-(4-Chloro-2-fluoro-6-methylphenyl)-4,4,5,5-tetramethyl-1,3,2-dioxaborolane

To a solution of crude 4-chloro-2-fluoro-6-methylaniline (0.25 g) from above step in MeCN (2.5 mL) was added tert-butyl nitrite (0.24 g, 2.30 mmol, 1.5 eq.) and bis(pinacolato)diboron (0.45 g, 1.80 mmol, 1.1 eq.). The reaction mixture was stirred for 2 h at 80° C. The mixture was diluted with water (20 mL) and extracted with DCM (20 mL×2). The organic layer was washed with brine, dried over anhydrous Na₂SO₄, and evaporated in vacuo. The residue was purified by flash chromatography on silica gel (PE:EA=20:1) to obtain 2-(4-chloro-2-fluoro-6-methylphenyl)-4,4,5,5-tetramethyl-1,3,2-dioxaborolane (0.5 g, containing bis(pinacolato)diboron) as yellow oil, which was used in the next step directly without further purification. ¹H NMR (400 MHz, DMSO-d6) δ 6.67 (d, J=1.8 Hz, 1H), 6.65-6.56 (m, 1H), 2.38 (s, 3H), 1.31 (s, 12H).

### Intermediate 11c: 2-(2-Fluoro-6-methyl-4-(trifluoromethyl) phenyl)-4,4,5,5-tetramethyl-1,3,2-dioxaborolane

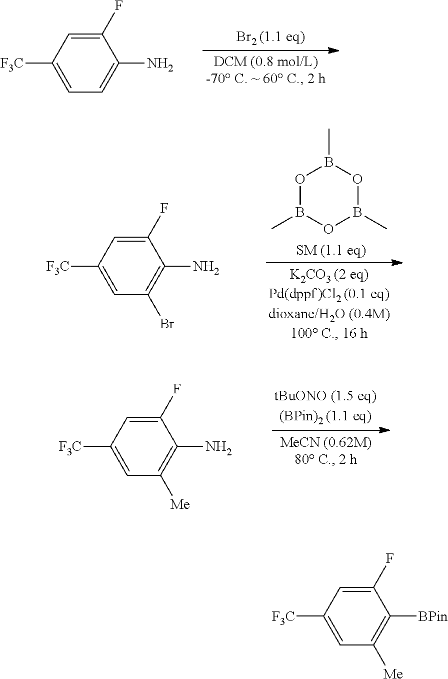

### Step 1. 2-Bromo-6-fluoro-4-(trifluoromethyl) aniline

To a solution of 1-(tert-butyl) 2-methyl (2S,4S)-4-hydroxypyrrolidine-1,2-dicarboxylate (7.00 g, 39.1 mmol, 1.0 eq.) in DCM (50 mL, 0.8 M) was added Br₂ (6.87 g, 43.0 mmol, 1.1 eq.) slowly at −70° C.˜−60° C. The mixture was stirred at −70° C.˜−60° C. for 2 h, then diluted with DCM, washed with brine, dried over sodium sulfate, filtered and concentrated in vacuo to give crude 2-bromo-6-fluoro-4-(trifluoromethyl) aniline as yellow oil, which was used in the next step without further purification. MS m/z 256.1, 257.9 [M−H]⁻.

### Step 2. 2-Fluoro-6-methyl-4-(trifluoromethyl) aniline

To a solution of crude 2-bromo-6-fluoro-4-(trifluoromethyl) aniline (2.0 g) from above step in 1,4-dioxane/H₂O (5:1, 50 mL) was added 2,4,6-trimethyl-1,3,5,2,4,6-trioxatriborinane (1.07 g, 8.50 mmol, 1.1 eq.), K₂CO₃ (2.10 g, 15.5 mmol, 2.0 eq.) and Pd(dppf)Cl₂ (0.57 g, 0.800 mmol, 0.1 eq.). The reaction mixture was stirred for 16 h at 100° C. The mixture was diluted with water (30 mL) and extracted with DCM (30 mL×2). The organic layer was washed with brine, dried over anhydrous Na₂SO₄ and evaporated in vacuo. The residue was concentrated in vacuo to give 2-fluoro-6-methyl-4-(trifluoromethyl) aniline (840 mg, crude) as yellow oil, which was used in the next step without further purification.

### Step 3. 2-(2-Fluoro-6-methyl-4-(trifluoromethyl) phenyl)-4,4,5,5-tetramethyl-1,3,2-dioxaborolane

To a solution of crude 2-fluoro-6-methyl-4-(trifluoromethyl) aniline (0.84 g) in MeCN (10 mL) was added tert-butyl nitrite (0.67 g, 6.5 mmol, 1.5 eq.) and bis(pinacolato)diboron (1.20 g, 4.70 mmol, 1.1 eq.). The reaction mixture was stirred for 2 h at 80° C., then diluted with water (50 mL) and extracted with DCM (80 mL×2). The organic layer was washed with brine, dried over anhydrous Na₂SO₄, and evaporated in vacuo to give crude methyl (2S,4S)-4-methylsulfonyloxy-1-trityl-pyrrolidine-2-carboxylate as yellow oil, which was used in the next step without further purification. ¹H NMR (400 MHz, DMSO-d6) δ: 7.43 (s, 1H), 7.38 (d, J=8.8 Hz, 1H), 2.46 (s, 3H), 1.33 (s, 12H).

### Intermediate 12: 2-(4-Chloro-5,6,7,8-tetrahydrophthalazin-1-yl)-5-(trifluoromethyl)phenol

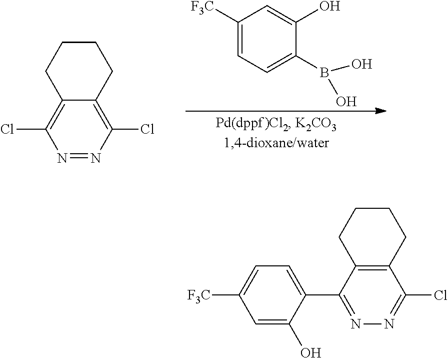 [SMILES: Clc1nnc(Cl)c2c1CCCC2 ; OB(O)c1ccc(C(F)(F)F)cc1O ; Oc1cc(C(F)(F)F)ccc1-c1nnc(Cl)c2c1CCCC2]

A mixture of 1,4-dichloro-5,6,7,8-tetrahydro-phthalazine (2.95 g, 14.5 mmol), [2-hydroxy-4-(trifluoromethyl)phenyl] boronic acid (3 g, 14.5 mmol), Pd(dppf)Cl₂ (1.07 g, 1.45 mmol) and K₂CO₃ (4.02 g, 29.1 mmol) in 1,4-dioxane (30 mL) and water (3 mL) was stirred at 100° C. for 16 h under N₂ atmosphere. After that, the mixture was diluted with water (20 ml), extracted with EtOAc (50 ml×3), dried over Na₂SO₄ and concentrated. The residue was purified by silica gel column (DCM:MeOH˜10%) to give 2-(4-chloro-5,6,7,8-tetrahydrophthalazin-1-yl)-5-(trifluoromethyl)phenol (2 g, 42% yield) as a red solid. MS m/z 329.0, 331.0 [M+H]⁺.

### Intermediate 13: 1-Chloro-4-(2-(methoxymethoxy)-4-(trifluoromethyl)phenyl)phthalazine

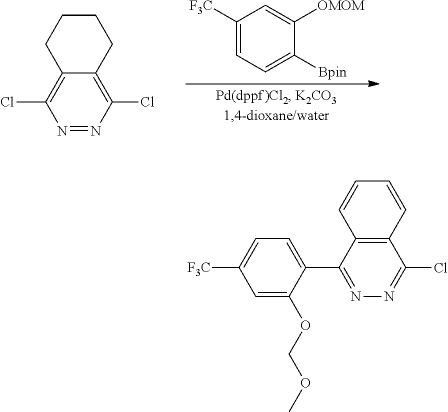

The title compound was prepared in analogous manner according to the procedure of Intermediate 12, using 1,4-dichlorophthalazine in place of 1,4-dichloro-5,6,7,8-tetrahydro-phthalazine. MS m/z 369.4, 371.0 [M+H]⁺.

### Intermediate 14a: 1-(2-(Methoxymethoxy)-4-(trifluoromethyl)phenyl)pyrido[3,4-d]pyridazin-4(3H)-one and

### Intermediate 14b: 4-Chloro-1-(2-(methoxymethoxy)-4-(trifluoromethyl)phenyl)pyrido[3,4-d]pyridazine

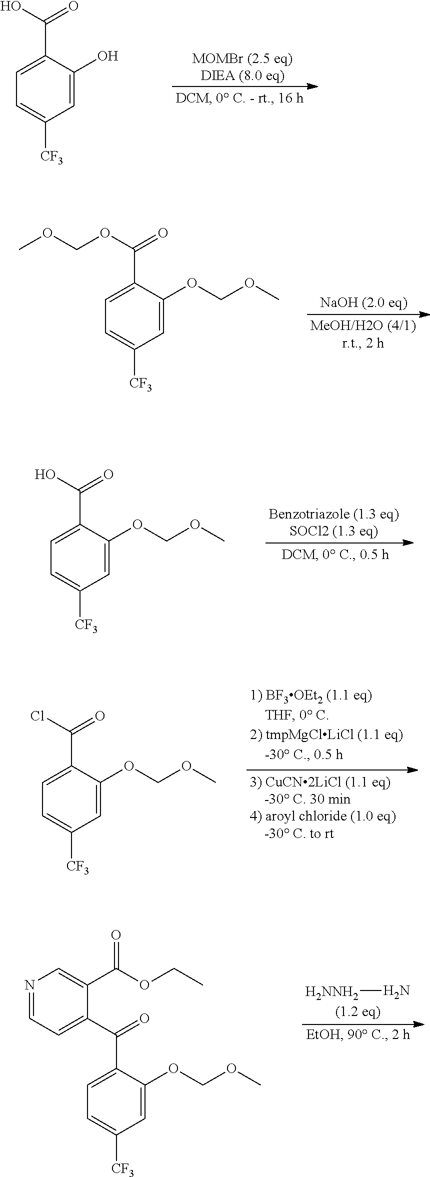

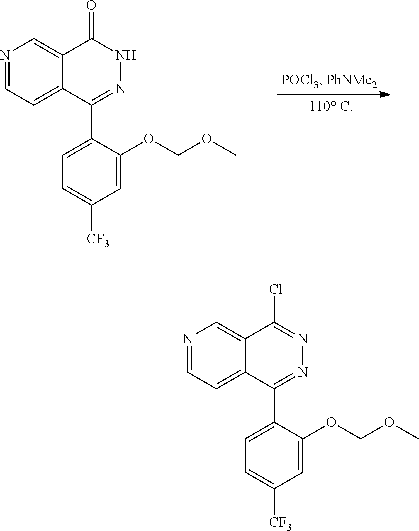 [SMILES: COCOc1cc(C(F)(F)F)ccc1-c1n[nH]c(=O)c2cnccc12 ; COCOc1cc(C(F)(F)F)ccc1-c1nnc(Cl)c2cnccc12]

### Step 1. Methoxymethyl 2-(methoxymethoxy)-4-(trifluoromethyl)benzoate

To a mixture of 2-hydroxy-4-(trifluoromethyl)benzoic acid (150 g, 727 mmol) in DCM (1.2 L) was added DIPEA (635 mL, 3640 mmol). The mixture was stirred at 0° C. and MOMBr (173 mL, 2185 mmol) was added dropwise. The resulting mixture was stirred at rt for 16 h. TLC (Petroleum ether/EtOAc 7:1) indicated the reaction was completed. The mixture was washed with water and brine. The organic phase was dried over anhydrous sodium sulfate, filtered and concentrated to provide title product (211 g, 99% yield) as light-yellow oil. ¹H NMR (400 MHz, DMSO-d6) δ 7.90 (d, J=8.0 Hz, 1H), 7.57 (s, 1H), 7.46 (d, J=8.0 Hz, 1H), 5.46 (s, 2H), 5.42 (s, 2H), 3.51 (s, 3H), 3.45 (s, 3H).

### Step 2. 2-(Methoxymethoxy)-4-(trifluoromethyl)benzoic acid

To a stirred solution of methoxymethyl 2-(methoxymethoxy)-4-(trifluoromethyl)benzoate (500 g, 1.7 mol) in MeOH (6 L) and water (1.5 L) was added NaOH (140 g, 3.5 mol) in portions at 0° C. The resulting mixture was stirred at rt for 2 h. After concentration to remove MeOH, the aqueous phase was acidified to pH=3 with HCl (1M). The precipitates were collected by filtration, washed with water and dried under vacuum to provide 2-(methoxymethoxy)-4-(trifluoromethyl)benzoic acid (400 g, 94% Yield) as a white solid. ¹H NMR (400 MHz, DMSO-d6) δ 13.33 (s, 1H), 7.80 (d, J=8.0 Hz, 1H), 7.51 (s, 1H), 7.42 (d, J=8.0 Hz, 1H), 5.36 (s, 2H), 3.42 (s, 3H).

### Step 3. 2-(Methoxymethoxy)-4-(trifluoromethyl)benzoyl chloride

A stock solution was prepared by dissolving 1H-indazole (38.7 g, 328 mmol), thionyl chloride (38.4 g, 323 mmol) in 200 mL DCM. Reaction was carried out by adding the stock solution intermittently to a stirred solution of 2-(methoxymethoxy)-4-(trifluoromethyl)benzoic acid (65 g, 259.8 mmol) in DCM (1 L). Before addition was complete, benzotriazole hydrochloride started precipitating out as a white solid. The mixture was stirred for another 0.5 h. After filtration, the filtrate was stirred with MgSO₄ 7H₂O (50 g) to destroy excess thionyl chloride. The white solid was filtered off and the filtrate was concentrated to give crude product (62 g, 88.8% Yield), which was used for the next step without further purification. ¹H NMR (400 MHz, CDCl₃) δ 8.08 (d, J=8.2 Hz, 1H), 7.50 (s, 1H), 7.36 (dd, J=8.3, 0.7 Hz, 1H), 5.33 (s, 2H), 3.54 (s, 3H).

### Step 4. Ethyl 4-(2-(methoxymethoxy)-4-(trifluoromethyl)benzoyl)nicotinate

Ethyl pyridine-3-carboxylate (130 g, 860 mmol) was dissolved in THE (950 mL) and boron trifluoride diethyl ether (135 g, 951 mmol) was added slowly at −40° C. After stirring for 0.5 h, 2,2,6,6-tetramethylpiperidinylmagnesium chloride lithium chloride complex solution (950 mL, 950 mmol, 1.0 mol/L in THF) was added slowly and the mixture was stirred for 30 min. Then fresh prepared copper cyanide lithium chloride complex solution in THE (CuCN (89.3 g, 997 mmol) and LiCl (82.84 g, 1954 mmol) in THE (950 mL) was stirred at 25° C. for 16 h) was added. After stirring for 0.5 h, 2-(methoxymethoxy)-4-(trifluoromethyl)benzoyl chloride (260 g, 968 mmol) in THE was added slowly and stirred for another 0.5 h. The mixture was warmed to rt and quenched with aqueous ammonium chloride solution and pH was adjusted to −8-9 with additional ammonia. The mixture was extracted with EA. The EA phase was washed with brine, dried over sodium sulfate, filtered and concentrated. The crude was purified by silica gel column (PE:EA=5:1 to 1:1 as eluent) to provide ethyl 4-(2-(methoxymethoxy)-4-(trifluoromethyl)benzoyl)nicotinate (158 g, 47.9% Yield) as yellow oil. MS m/z 384.2 [M+H]⁺. ¹H NMR (400 MHz, DMSO-d6) δ 9.12 (s, 1H), 8.90 (d, J=5.0 Hz, 1H), 8.08 (d, J=8.1 Hz, 1H), 7.54 (d, J=8.4 Hz, 1H), 7.51 (d, J=5.0 Hz, 1H), 7.43 (s, 1H), 5.00 (s, 2H), 4.15 (q, J=7.1 Hz, 2H), 3.02 (s, 3H), 1.07 (t, J=7.1 Hz, 3H).

### Step 5. 1-(2-(Methoxymethoxy)-4-(trifluoromethyl)phenyl)pyrido[3,4-d]pyridazin-4(3H)-one (14a)

To a solution of ethyl ethyl 4-(2-(methoxymethoxy)-4-(trifluoromethyl)benzoyl)nicotinate (50 g, 130 mmol) in EtOH (500 mL) was added hydrazine (7.8 g, 156 mmol) and the mixture was stirred at 90° C. for 2 h. After cooling to rt, the precipitate was filtered and the filter cake was washed with EtOH and dried under vacuum to obtain 1-(2-(methoxymethoxy)-4-(trifluoromethyl) phenyl)pyrido[3,4-d]pyridazin-4(3H)-one (36 g, 78.5% Yield) as white solid. MS m/z 352.0 [M+H]⁺. ¹H NMR (400 MHz, DMSO-d6) δ 9.52 (s, 1H), 8.96 (d, J=5.5 Hz, 1H), 7.68 (d, J=7.8 Hz, 1H), 7.61 (s, 1H), 7.56 (d, J=8.0 Hz, 1H), 7.25 (d, J=5.5 Hz, 1H), 5.24 (d, J=12.6 Hz, 2H), 3.21 (s, 3H).

### Step 6. 4-Chloro-1-(2-(methoxymethoxy)-4-(trifluoromethyl)phenyl)pyrido[3,4-d]pyridazine (14b)

A mixture of 1-(2-(methoxymethoxy)-4-(trifluoromethyl)phenyl)pyrido[3,4-d]pyridazin-4(3H)-one (50 g, 142 mmol) and N,N-dimethylaniline (68.7 g, 568 mmol) in POCl₃ (434 g, 2.84 mol) was stirred for 30 min at 110° C. After cooling to rt, the mixture was concentrated to remove excess of POCl₃, the residue was adjusted to pH=7-8 with TEA and purified by silica gel column chromatography (EA:PE=1:3) to provide 4-chloro-1-(2-(methoxymethoxy)-4-(trifluoromethyl) phenyl)pyrido[3,4-d]pyridazine (22 g, 41.8% yield) as light yellow solid. MS m/z 370.0 [M+H]⁺; ¹H NMR (400 MHz, DMSO-d6) δ 9.80 (d, J=0.9 Hz, 1H), 9.13 (d, J=5.7 Hz, 1H), 7.75 (d, J=7.7 Hz, 1H), 7.67 (s, 1H), 7.65-7.60 (m, 2H), 5.22 (d, J=20.0 Hz, 2H), 3.16 (s, 3H).

### Intermediate 15a: (3R)-1-[2-[tert-Butyl(dimethyl)silyl]oxyethyl]piperidin-3-amine

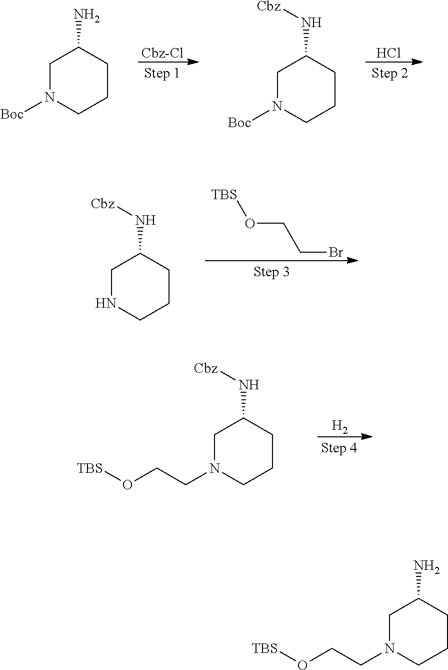

### Step 1. tert-Butyl (3R)-3-(benzyloxycarbonylamino)piperidine-1-carboxylate

To a solution of tert-butyl (R)-3-aminopiperidine-1-carboxylate (30 g, 0.15 mol) in 330 ml THE and 83 ml water was added Na₂CO₃(43 g, 0.31 mol). And then Cbz-Cl (34, 0.199 mol) was added dropwised into the mixture with ice bath, monitored by TLC. After 5 hours, the reaction was extracted with EA, dried over Na₂SO₄ and evaporated in vacuo. The crude product was purified via flash chromatography (PE:EA=4:1) to give tert-butyl (3R)-3-(benzyloxycarbonylamino) piperidine-1-carboxylate (35 g, 84% Yield). MS m/z 235.1 [M-Boc+H]⁺

### Step 2. Benzyl N-[(3R)-3-piperidyl]carbamate

A solution of tert-butyl (3R)-3-(benzyloxycarbonylamino)piperidine-1-carboxylate (35 g, 0.104 mol) in 450 mL DCM was added HCl (350 mL, 4M in 1,4-dioxane), monitored by TLC. After 2 h, the solvent was removed in vacuo to give crude benzyl N-[(3R)-3-piperidyl]carbamate (25 g, 0.106 mol, 100% yield) which was used for next step without purification. MS m/z 234.1 [M+H]⁺.

### Step 3. Benzyl N-[(3R)-1-[2-[tert-butyl(dimethyl)silyl]oxyethyl]-3-piperidyl]carbamate

To a solution of benzyl N-[(3R)-3-piperidyl]carbamate (25 g, 0.106 mol) in CH₃CN (680 mL) was added Cs₂CO₃ (175 g, 0.537 mol) and (2-bromoethoxy)(tert-butyl)dimethylsilane (40 g, 0.167 mol). The reaction was stirred 90° C. for 16 h. After reaction, the mixture was filtered and the solvent was removed in vacuo, then crude product was purified via flash chromatography to give the product benzyl N-[(3R)-1-[2-[tert-butyl(dimethyl)silyl]oxyethyl]-3-piperidyl]carbamate (22.5 g, 0.057 mol, 53% Yield). MS m/z 393.2 [M+H]⁺.

### Step 4. (3R)-1-[2-[tert-Butyl(dimethyl)silyl]oxyethyl]piperidin-3-amine

To a solution of benzyl N-[(3R)-1-[2-[tert-butyl(dimethyl)silyl]oxyethyl]-3-piperidyl]carbamate (22.5 g, 0.057 mol) in MeOH (60 mL) was added Pd/C (4.5 g, 20%). The system was evacuated and refilled with hydrogen. Then the mixture stirred at R.T overnight. After reaction, the mixture was filtered, the solvent was removed in vacuo and the crude product was purified via flash chromatography to give the product (3R)-1-[2-[tert-butyl(dimethyl)silyl]oxyethyl]piperidin-3-amine (12 g, 81% Yield). MS m/z 259.2 [M+H]⁺. ¹H NMR (400 MHz, CDCl₃) δ 3.68 (t, J=6.6 Hz, 2H), 2.85-2.74 (m, 2H), 2.67-2.58 (m, 1H), 2.46 (t, J=6.4 Hz, 2H), 2.11-2.01 (m, 1H), 1.94-1.83 (m, 1H), 1.83-1.71 (m, 4H), 1.68-1.58 (m, 1H), 1.56-1.43 (m, 1H), 1.09-0.96 (m, 1H), 0.88-0.79 (m, 9H), 0.04-(−)0.04 (m, 6H).

### Intermediate 15b: (R)-1-Ethylpiperidin-3-amine hydrochloride

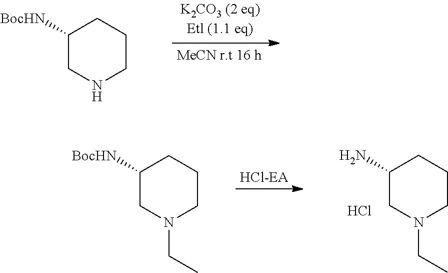

### Step 1. tert-Butyl (R)-(1-ethylpiperidin-3-yl)carbamate

To a solution of tert-butyl (R)-piperidin-3-ylcarbamate (4 g, 20 mmol) in MeCN (40 mL) was added K₂CO₃ (2.76 g, 20 mmol) and MeI (3.12 g, 20 mmol) at 20° C. under N₂. The mixture was stirred at 20° C. for 16 hours. TLC showed the reaction was completed. The reaction mixture was poured into water (100 mL) and extracted with EA (100 mL×2). The combined organic layers were washed with brine (100 mL×2). dried over Na₂SO₄, filtered and concentrated. Purified by column (DCM:MeOH=0-10%) to give tert-butyl (R)-(1-ethylpiperidin-3-yl)carbamate (3.6 g, 15.8 mmol, 78.9% yield) as yellow oil. ¹H NMR (400 MHz, DMSO-d6) δ 6.65 (d, J=7.8 Hz, 1H), 3.32 (s, 1H), 2.70 (dd, J=41.0, 9.7 Hz, 2H), 2.29 (q, J=7.1 Hz, 2H), 1.83-1.53 (m, 4H), 1.48-1.40 (m, 1H), 1.37 (s, 9H), 1.11 (qd, J=11.8, 3.7 Hz, 1H), 0.96 (t, J=7.2 Hz, 3H).

### Step 2. (R)-1-Ethylpiperidin-3-amine hydrochloride

To a solution of tert-butyl (R)-(1-ethylpiperidin-3-yl)carbamate (3.6 g, 15.8 mmol) in MeOH (10 mL) was added HCl (30 mL, 3 M in EA) at RT. The mixture was stirred at 20° C. for 16 hours. TLC showed the reaction was completed. The reaction mixture was concentrated to give (R)-1-ethylpiperidin-3-amine hydrochloride (3.03 g, 15.0 mmol, 95.0% yield) as yellow oil. MS m/z 129.2 [M+H]⁺; ¹H NMR (400 MHz, CD₃OD) δ 3.83-3.59 (m, 3H), 3.36-3.30 (m, 2H), 3.18-2.96 (m, 2H), 2.25 (d, J=12.7 Hz, 1H), 2.15 (d, J=14.9 Hz, 1H), 2.07-1.92 (m, 1H), 1.76-1.72 (m, 1H), 1.43 (t, J=7.3 Hz, 3H).

### Intermediate 15c: (R)-1-(Cyclobutyl-1-d)piperidin-3-amine

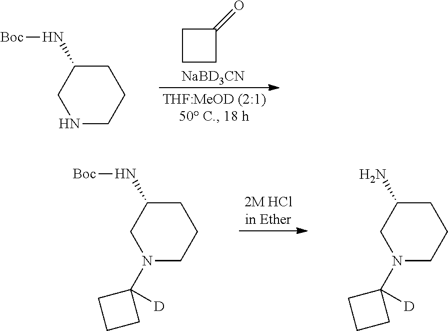

### Step 1: tert-Butyl (R)-(1-(cyclobutyl-1-d)piperidin-3-yl)carbamate

A mixture of tert-butyl (R)-piperidin-3-ylcarbamate (200 mg, 1.0 mmol, 1.0 eq.), sodium cyanoborodeuteride (200 mg, 3.0 mmol, 3.0 eq.) was dissolved in 1.2 mL of THF: MeOD (5:1) mixture followed by the addition of cyclobutanone (150 mg, 2.1 mmol). The reaction mixture was then stirred at 50° C. for 18 h. The crude reaction mixture was allowed to cool to room temperature and diluted with DCM (10 mL). The organic phase was washed with saturated aqueous NaHCO₃ solution (2 mL×3) followed by brine and water. The organic phase was dried over MgSO₄, filtered, and concentrated in vacuo. The crude material was used for the following step without further purification. MS m/z 256.2 [M+H]⁺.

### Step-2: (R)-1-(Cyclobutyl-1-d)piperidin-3-amine

The crude compound from previous step was dissolved in DCM (5 mL) and then 2M HCl solution in ether (3 mL) was added slowly at rt while the mixture was vigorously stirred. The reaction mixture was stirred at rt overnight and then concentrated in vacuo to afford a yellow solid (138 mg) of HCl salt. The amine was used without further purification. MS m/z 156.2 [M+H]⁺.

The intermediates below were prepared according to the procedure of Intermediate 15b or c by substituting the appropriate starting materials, reagents and reaction conditions.

| Structure | Spectral Data |
|---|---|
| 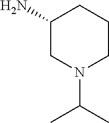 | MS m/z 243.2 [M + H]⁺; ¹H NMR (400 MHz, CD₃OD) δ 3.83-3.72 (m, 1H), 3.71- 3.61 (m, 2H), 3.56-3.48 (m, 1H), 3.15-3.03 (m, 2H), 2.30-2.19 (m, 1H), 2.18- 2.10 (m, 1H), 2.09-1.97 (m, 1H), 1.81-1.66 (m, 1H), 1.43 (d, J = 6.7 Hz, 6H). |
| 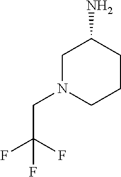 [SMILES: N[C@@H]1CCCN(CC(F)(F)F)C1] | MS m/z 183.2 [M + H]⁺; ¹H NMR (400 MHz, CD₃OD) δ 4.00 (q, J = 9.3 Hz, 2H), 3.65 (ddd, J = 22.7, 13.2, 8.3 Hz, 2H), 3.39 (d, J = 12.2 Hz, 1H), 3.26-3.06 (m, 2H), 2.09 (dtd, J = 23.9, 8.6, 4.2 Hz, 2H), 2.01-1.85 (m, 1H), 1.72 (dtd, J = 13.8, 10.1, 3.7 Hz, 1H). |
| 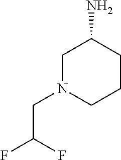 [SMILES: N[C@@H]1CCCN(CC(F)F)C1] | ¹H NMR (400 MHz, CD₃OD) δ 6.48 (tt, J = 53.5, 3.7 Hz, 1H), 3.84-3.64 (m, 4H), 3.59 (d, J = 11.9 Hz, 1H), 3.22 (dt, J = 12.2, 7.1 Hz, 2H), 2.15 (dtd, J = 15.1, 7.9, 3.8 Hz, 2H), 2.07-1.93 (m, 1H), 1.71 (ddd, J = 15.2, 11.9, 3.8 Hz, 1H). |
| 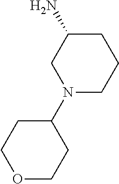 [SMILES: N[C@@H]1CCCN(C2CCOCC2)C1] | MS m/z 185.2 [M + H]⁺ |
| 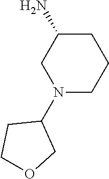 [SMILES: N[C@@H]1CCCN(C2CCOC2)C1] | MS m/z 171.2 [M + H]⁺ |

### Intermediate 15d: (R)-1-(3-Aminopiperidin-1-yl)-2-methylpropan-2-ol

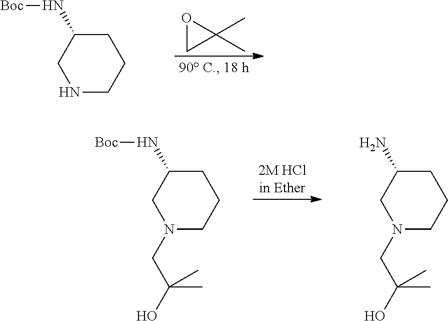

### Step-1: tert-Butyl (R)-(1-(2-hydroxy-2-methylpropyl)piperidin-3-yl)carbamate

A mixture of tert-butyl (R)-piperidin-3-ylcarbamate (700 mg, 3.5 mmol, 1.0 eq.), and 2,2-dimethyloxirane (800 mg, 11.1 mmol, 3.2 eq.) was stirred at 90 C for 18 h. The crude reaction mixture was allowed to cool to room temperature and diluted with DCM (20 mL). The organic phase was washed with saturated aqueous NaHCO₃ solution (4 mL×2) followed by brine and water. The organic phase was dried over MgSO₄, filtered, and concentrated in vacuo. The crude material was used then for the following step. MS m/z 273.3 [M+H]⁺.

### Step-2: (R)-1-(3-Aminopiperidin-1-yl)-2-methylpropan-2-ol

The crude compound from previous step was dissolved in DCM (5 mL) and then 2M HCl solution in ether (10 mL) was added slowly at rt while the mixture was vigorously stirred. The reaction mixture was stirred at rt for overnight and concentrated in vacuo to afford a light brown solid (520 mg) of HCl salt. The amine was used without further purification. MS m/z 173.2 [M+H]⁺.

### Intermediate 16a: (3R,5R)-5-Fluoro-1-methylpiperidin-3-amine hydrochloride

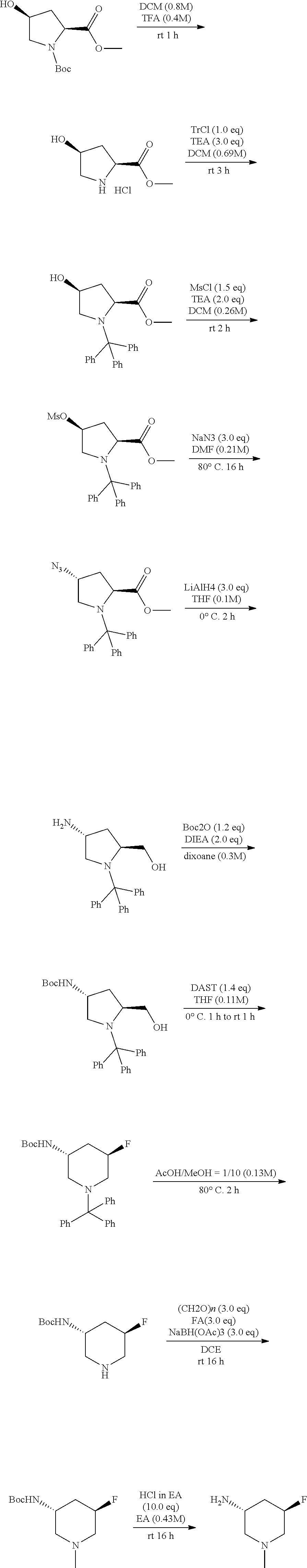

### Step 1. Methyl (2S,4S)-4-hydroxypyrrolidine-2-carboxylate

1-(tert-Butyl) 2-methyl (2S,4S)-4-hydroxypyrrolidine-1,2-dicarboxylate (10.0 g, 40.8 mmol) was dissolved in DCM (50 mL) and cooled to 0° C. TFA (10 mL, 132.3 mmol) was slowly added and the mixture was warmed to room temperature and stirred for 1 h. The mixture was concentrated in vacuo to give crude methyl (2S,4S)-4-hydroxypyrrolidine-2-carboxylate as yellow oil, which was used to next step without further purification. MS m/z 146.1 [M+H]⁺.

### Step 2. Methyl (2S,4S)-4-hydroxy-1-tritylpyrrolidine-2-carboxylate

To a solution of crude methyl (2S,4S)-4-hydroxypyrrolidine-2-carboxylate from above step in DCM (50 mL) was added TEA (12.4 g, 123 mmol) and Ph₃CCl (11.4 g, 40.9 mmol). The reaction mixture was stirred for 2 h at rt. The mixture was poured into water and extracted with DCM (30 mL×2). The organic layer was washed with brine, dried over anhydrous Na₂SO₄, and evaporated in vacuo. The residue was purified by flash chromatography on silica gel (PE:EA=10:1 to 5:1) to obtain methyl (2S,4S)-4-hydroxy-1-trityl-pyrrolidine-2-carboxylate (7.60 g, 20.0 mmol, 48% Yield for two steps) as a white solid. ¹H NMR (400 MHz, CDCl₃) δ 7.60-7.50 (m, 6H), 7.32-7.23 (m, 6H), 7.21-7.13 (m, 3H), 3.92-3.80 (m, 3H), 3.65 (s, 3H), 3.46 (d, J=11.5 Hz, 1H), 2.80 (dd, J=11.5, 3.8 Hz, 1H), 1.62 (d, J=13.8 Hz, 1H), 1.38-1.11 (m, 1H).

### Step 3. Methyl (2S,4S)-4-((methylsulfonyl)oxy)-1-tritylpyrrolidine-2-carboxylate

To a solution of methyl (2S,4S)-4-hydroxy-1-trityl-pyrrolidine-2-carboxylate (10.0 g, 25.8 mmol) in DCM (100 mL) was added TEA (7.90 g, 78.0 mmol) and MsCl (4.50 g, 39.0 mmol). The reaction mixture was stirred for 2 h at rt. The mixture was diluted with water and extracted with DCM (80 mL×2). The organic layer was washed with brine, dried over anhydrous Na₂SO₄, and evaporated in vacuo to give crude methyl (2S,4S)-4-methylsulfonyloxy-1-trityl-pyrrolidine-2-carboxylateas yellow oil, which was used in the next step directly.

### Step 4. Methyl (2S,4R)-4-azido-1-tritylpyrrolidine-2-carboxylate

To a solution of methyl (2S,4S)-4-methylsulfonyloxy-1-trityl-pyrrolidine-2-carboxylate from above step in DMF (140 mL) was added sodium azide (7.80 g, 120 mmol). The reaction mixture was stirred for 16 h at 80° C. The reaction mixture was diluted with water and extracted with EA (3×100 mL). The combined organic layers were washed with water, brine, dried over anhydrous Na₂SO₄ and concentrated. The residue was purified by flash chromatography on silica gel (PE:EA=0%˜10%) to give methyl (2S,4R)-4-azido-1-trityl-pyrrolidine-2-carboxylate (8.00 g, 19.4 mmol, 64.5% Yield for two steps) as yellow oil. ¹H NMR (400 MHz, CDCl₃) δ 7.58-7.51 (m, 6H), 7.33-7.24 (m, 6H), 7.18 (t, J=7.3 Hz, 3H), 4.20-4.06 (m, 1H), 3.91 (d, J=8.9 Hz, 1H), 3.72 (dd, J=10.3, 7.6 Hz, 1H), 3.63 (s, 3H), 2.63 (dd, J=10.3, 7.8 Hz, 1H), 1.90-1.79 (m, 1H), 0.94-0.88 (m, 1H).

### Step 5. ((2S,4R)-4-Amino-1-tritylpyrrolidin-2-yl)methanol

To a stirred suspension of LiAlH4 (2.20 g, 58.0 mmol) in THE (80 mL) at 0° C. was added a solution of a solution of methyl (2S,4R)-4-azido-1-trityl-pyrrolidine-2-carboxylate (8.00 g, 19.4 mmol) in THE (10 mL). After the addition was completed, the reaction mixture was stirred at 0° C. for 1 h. The reaction mixture was quenched with water (2.5 mL), 15% NaOH solution (2.5 mL) and water (6 mL). After stirring for 0.5 h, the mixture was dried over Na₂SO₄, filtered and concentrated to give crude product [(2S,4R)-4-amino-1-trityl-pyrrolidin-2-yl]methanol as yellow oil (7.00 g, 19.5 mmol, >99% Yield), which was used to the next step without purification.

### Step 6. tert-Butyl ((3R,5S)-5-(hydroxymethyl)-1-tritylpyrrolidin-3-yl)carbamate

To a solution of [(2S,4R)-4-amino-1-trityl-pyrrolidin-2-yl]methanol (7.00 g, 19.5 mmol) in 1,4-dioxane (70 mL) was added TEA (3.90 g, 39.0 mmol) and (Boc)₂O (5.10 g, 23.0 mmol). The mixture was stirred for 2 h at rt, then diluted with water and extracted with EA (3×100 mL). The combined organic layers were washed with water, brine, dried over anhydrous Na₂SO₄ and concentrated. The residue was purified by silica gel column chromatography (PE:EA=20:1) to give tert-butyl N-[(3R,5S)-5-(hydroxymethyl)-1-trityl-pyrrolidin-3-yl]carbamate (8.00 g, 17.4 mmol, 89.3% Yield) as colorless oil. MS m/z 459.3 [M+H]⁺, ¹H NMR (400 MHz, CDCl₃) δ 7.67-7.50 (m, 6H), 7.33-7.23 (m, 7H), 7.18 (t, J=7.3 Hz, 3H), 4.27-4.18 (m, 1H), 3.77-3.64 (m, 2H), 3.58-3.51 (m, 1H), 3.50-3.32 (m, 2H), 2.60-2.48 (m, 1H), 2.03-1.94 (m, 1H), 1.90-1.81 (m, 1H), 1.36 (s, 9H).

### Step 7. tert-Butyl ((3R,5R)-5-fluoro-1-tritylpiperidin-3-yl)carbamate

DAST (2.80 mL, 21.0 mmol) was added dropwise to a stirred solution of tert-butyl N-[(3R,5S)-5-(hydroxymethyl)-1-trityl-pyrrolidin-3-yl]carbamate (6.80 g, 15.0 mmol) in THE (70 mL) at 0° C. The mixture was stirred for 1 h at 0° C. and 1 h at RT, then cooled to 0° C. again. A saturated aqueous solution of Na₂CO₃ was added to adjust pH to 12. The phases were separated and the aqueous phase was extracted with EtOAc (2×40 mL). The organic fractions were combined, dried over Na₂SO₄, filtered, and concentrated under reduced pressure. The crude material was purified by flash chromatography (silica gel, PE/EtOAc=20/1) to afford tert-butyl N-[(3R,5R)-5-fluoro-1-trityl-3-piperidyl]carbamate (3.90 g, 8.50 mmol 57% Yield) as a white solid. MS m/z 483.3 [M+Na]+,

### Step 8. tert-Butyl ((3R,5R)-5-fluoropiperidin-3-yl)carbamate

To a solution of tert-butyl N-[(3R,5R)-5-fluoro-1-trityl-3-piperidyl]carbamate (3.90 g, 8.50 mmol) in MeOH (80 mL) was added AcOH (8 mL, 139.6 mmol). The mixture was stirred for 2 h at 80° C., then concentrated under reduced pressure. The residue was diluted with EA (20 mL) and H₂O (20 mL). The mixture was acidified with aqueous HCl till pH=2-3 and extracted with EA (10 mL×2). The aqueous layers were basified with aqueous K₂CO₃ till pH=9-10 and extracted with DCM (50 mL×3). The DCM layers were combined and dried over Na₂SO₄, filtered and concentrated under reduced pressure to provide tert-butyl N-[(3R,5R)-5-fluoro-3-piperidyl]carbamate (1.60 g, 7.30 mmol 87% Yield) as a white solid. MS m/z 219.1 [M+H]⁺, ¹H NMR (400 MHz, DMSO-d6) δ 6.61 (s, 1H), 4.77 (d, J=48.5 Hz, 1H), 3.60 (br s, 1H), 2.96-2.82 (m, 2H), 2.77-2.62 (m, 1H), 2.43-2.33 (m, 1H), 2.09-1.97 (m, 1H), 1.78-1.55 (m, 1H), 1.39 (s, 9H)

### Step 9. tert-Butyl ((3R,5R)-5-fluoro-1-methylpiperidin-3-yl)carbamate

To a solution of tert-butyl N-[(3R,5R)-5-fluoro-3-piperidyl]carbamate (1.60 g, 7.30 mmol) in DCE (20 mL) was added formic acid (1.20 g, 26.0 mmol) and (HCHO)₃ (0.75 g, 25 mmol) and stirred for 1 h at rt. To the mixture was added NaBH(OAc)₃ (5.4 g, 25.0 mmol) and stirred for 16 h at rt. The reaction mixture was quenched with water, basified with aqueous K₂CO₃ solution and extracted with DCM. The organic phase was combined and washed with brine. The organic phase was dried over Na₂SO₄ and concentrated in vacuum. The residue was purified by silica gel chromatography (PE/EA=3/1) to afford tert-butyl N-[(3R,5R)-5-fluoro-1-methyl-3-piperidyl]carbamate (1.00 g, 4.30 mmol, 59.0% Yield) as a white solid. MS m/z 233.1 [M+H]⁺,

### Step 10. (3R,5R)-5-Fluoro-1-methylpiperidin-3-amine hydrogen chloride

To a solution of tert-butyl N-[(3R,5R)-5-fluoro-1-methyl-3-piperidyl]carbamate (1.00 g, 4.30 mmol) in EA (10 mL) was added HCl in 1,4-dioxane (14 mL, 56 mmol, 4 mol/L). The resulting mixture was stirred at room temperature for 2 h, then filtered and the solid was wash with EA (2 mL) and dried under vacuum to give (3R,5R)-5-fluoro-1-methyl-piperidin-3-amine (750 mg, 5.67 mmol, 75% Yield) as a white solid. MS m/z 133.1 [M+H]⁺, ¹H NMR (400 MHz, DMSO-d6) δ: 11.18 (d, J=3.6 Hz, 1H), 8.94 (s, 3H), 5.25 (d, J=44.4 Hz, 1H), 3.72-3.66 (m, 2H), 3.56-3.50 (m, 1H), 3.41-3.27 (m, 1H), 3.13 (t, J=11.2 Hz, 1H), 2.85 (s, 3H), 2.47-2.41 (m, 1H), 2.01-1.83 (m, 1H).

### Intermediate 16b: (3R,5R)-5-Fluoro-1-ethylpiperidin-3-amine hydrochloride

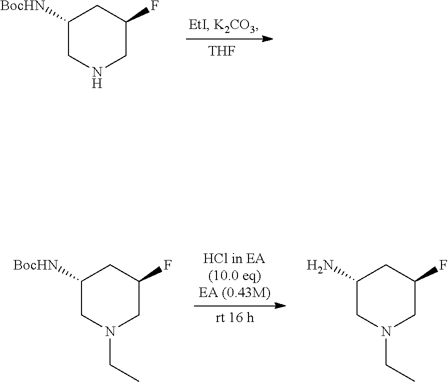

The starting material, tert-butyl ((3R,5R)-5-fluoropiperidin-3-yl)carbamate, was prepared as the procedure of Intermediate 16a step 1 to 8.

The title compound was prepared in analogous manner according to the procedure of Intermediate 15b, using tert-butyl ((3R,5R)-5-fluoropiperidin-3-yl)carbamate in place of tert-butyl (R)-piperidin-3-ylcarbamate in step 1. MS m/z 147.2 [M+H]⁺; ¹H NMR (400 MHz, DMSO-d6) δ: 11.02 (s, 1H), 8.94 (s, 3H), 5.27 (d, J=44.8 Hz, 1H), 3.74-3.62 (m, 3H), 3.32-3.22 (m, 3H), 3.09-3.05 (m, 1H), 2.48-2.43 (m, 1H), 2.06-1.89 (m, 1H), 1.28-1.25 (t, J=14.0 Hz, 3H).

### Intermediate 17: tert-Butyl 2-(aminomethyl)azetidine-1-carboxylate

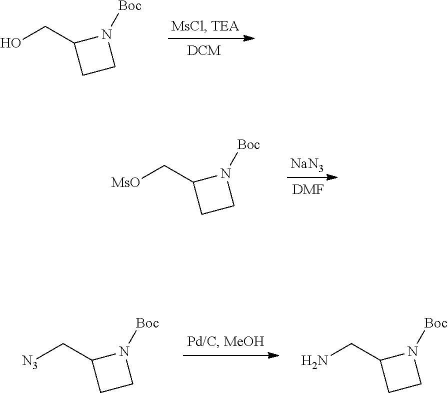

### Step 1. tert-Butyl 2-(methylsulfonyloxymethyl)azetidine-1-carboxylate

To a solution of tert-butyl 2-(hydroxymethyl)azetidine-1-carboxylate (1000 mg, 5.34 mmol) and triethylamine (820 mg, 8.10 mmol) in dichloromethane (10 mL, 156.0 mmol) at 0° C. was added methanesulfonyl chloride (800 mg, 6.98 mmol). This mixture was stirred at r.t for 2 hour, monitored by TLC and LCMS. The mixture was then poured into H₂O, extracted with CH₂Cl₂ (30*2 ml). The combined organic was dried over Na₂SO₄ and concentrated under vacuum to get crude tert-butyl 2-(methylsulfonyloxymethyl)azetidine-1-carboxylate (900 mg, 64% Yield) which was used in the next reaction directly. MS m/z 166.2 [M-Boc+H]⁺.

### Step 2. tert-Butyl 2-(azidomethyl)azetidine-1-carboxylate

To a solution of tert-butyl 2-(methylsulfonyloxymethyl)azetidine-1-carboxylate (800 mg, 3.02 mmol) in N,N-dimethylformamide (10 mL, 129 mmol) was added sodium azide (300 mg, 4.6 mmol) successively. This mixture was stirred at 60° C. overnight, monitored by TLC and LCMS. The mixture was poured into H₂O, extracted with EA (2×30 ml). The combined organic was dried over Na₂SO₄ and concentrated under vacuum to give crude tert-butyl 2-(azidomethyl)azetidine-1-carboxylate (700 mg, 84% Yield) which was used in next reaction directly. MS m/z 235.3 [M+Na]⁺.

### Step 3. tert-Butyl 2-(aminomethyl)azetidine-1-carboxylate

To a solution of tert-butyl 2-(azidomethyl)azetidine-1-carboxylate (700 mg, 3.30 mmol) in methanol (10 mL, 247 mmol) was added Pd/C (70 mg, 0.066 mmol). Then the mixture was stirred overnight at RT under H₂ atmosphere, monitored by TLC and LCMS. After reaction, Pd/C was filtrated out and the filtrate was concentrated under vacuum to give crude tert-butyl 2-(aminomethyl)azetidine-1-carboxylate (500 mg, 81% Yield) which was used in the next reaction directly. MS m/z 187.1 [M+H]⁺.

### Intermediate 18: (R)-1-(2-(Difluoromethoxy)ethyl)piperidin-3-amine hydrochloride

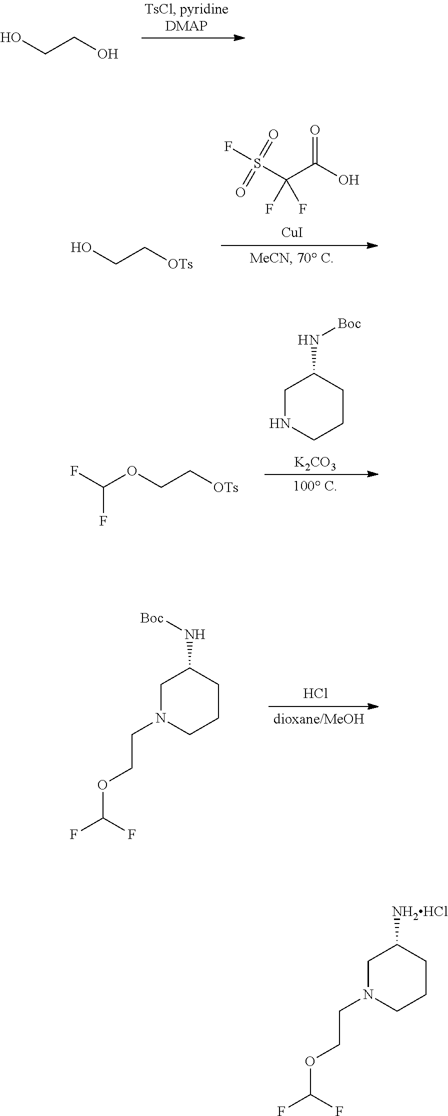

### Step 1: 2-Hydroxyethyl 4-methylbenzenesulfonate

A mixture of ethylene glycol (2.5 g, 40 mmol), TsCl (1.90 g, 10 mmol), pyridine (1.1 g, 12 mmol) and DMAP (12 mg, 0.1 mmol) was stirred at rt for 18 h. The reaction mixture was partitioned between DCM (20 mL) and 0.5 M hydrochloric acid. The organic layer was dried (Na₂SO₄), filtered, and concentrated. Purification by chromatography on SiO₂ (EtOAc:hexanes, 0 to 60%) gave a colorless oil (1.32 g, 78%). ¹H NMR (400 MHz, CDCl₃) δ 7.82 (d, J=8.25 Hz, 2H), 7.37 (d, J=8.13 Hz, 2H), 4.15 (t, J=4.50 Hz, 2H), 3.83 (t, J=4.63 Hz, 2H), 2.46 (s, 3H), 2.00 (br s, 1H).

### Step 2. 2-(Difluoromethoxy)ethyl 4-methylbenzenesulfonate

To a stirred solution of 2-hydroxyethyl 4-methylbenzenesulfonate (1.70 g, 7.86 mmol) in MeCN (13 mL) was added copper (I) iodide (0.300 g, 1.57 mmol). The resulting mixture was stirred at 70° C. and treated with 2,2-difluoro-2-fluorosulfonyl-acetic acid (2.80 g, 15.7 mmol) as a solution in MeCN (10 mL) dropwise over a period of 25 min. The resulting mixture was treated with anhydrous Na₂SO₄ (small scoop) and stirring continued for 1.5 h. The mixture was then cooled to rt, diluted with Et₂O and washed with brine, a 1:1 mixture of brine: water (2×), and brine. The organic phase was dried (Na₂SO₄), filtered and concentrated. Purification by chromatography on SiO₂ (EtOAc:hexanes, 0-25%) gave a pale yellow oil (0.759 g, 36%). ¹H NMR (400 MHz, CDCl₃) δ 7.79 (d, J=8.25 Hz, 2H), 7.49 (d, J=8.13 Hz, 2H), 6.65 (t, J=75.04 Hz, 1H), 4.22-4.16 (m, 2H), 4.04-3.98 (m, 2H), 2.43 (s, 3H)

### Step 3 and 4: (R)-1-(2-(Difluoromethoxy)ethyl)piperidin-3-amine hydrochloride

t-Butyl N-[(3R)-3-piperidyl]carbamate (0.500 g, 2.50 mmol) in DMF (10 mL) was added 2-(difluoromethoxy)ethyl 4-methylbenzenesulfonate (0.764 g, 2.87 mmol) and K₂CO₃ (0.690 g, 4.99 mmol). The mixture was stirred at 100° C. for 2 h. The reaction was diluted with DCM/iPrOH (9:1) and washed with water, brine, dried over Na₂SO₄, filtered and concentrated. Purification by chromatography on SiO₂ (EtOAc:hexanes, 5 to 60%) gave a pale yellow oil (0.709 g). The oil was dissolved in MeOH (2.0 mL), treated with HCl/dioxane (4.0 M, 3 mL) and stirred for 4 h. The mixture was concentrated, resuspended in ether and filtered to give a white solid (0.371 g, 67%). ¹H NMR (400 MHz, D₂O) δ 6.45 (t, J=73.6 Hz, 1H), 4.26 (t, J=5.2 Hz, 2H), 3.81 (d, J=11.2 Hz, 1H), 3.67-3.58 (m, 2H), 3.55 (t, J=4.8 Hz, 2H), 3.15-3.02 (m, 2H), 2.21 (d, J=12.4 Hz, 1H), 2.11 (d, J=15.2 Hz, 1H), 1.90-1.79 (m, 1H), 1.70-1.60 (m, 1H).

### Intermediate 19a: (R)-4-Chloro-N-(1-methylpiperidin-3-yl)phthalazin-1-amine

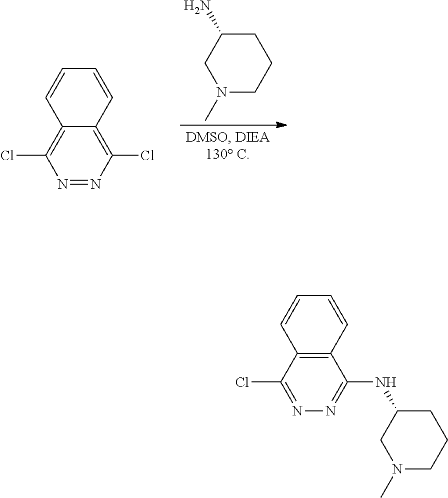

To a solution of 1,4-dichlorophthalazine (200 mg, 1.0048 mmol) in 1-methyl-2-pyrrolidinone (3 mL) was added (3R)-1-methylpiperidin-3-amine (1.5 eq., 1.5 mmol) and N,N-diisopropylethylamine (3.0 eq., 3.0 mmol). The resulting mixture was heated to 100° C. and kept stirring at that temperature for 12 h. Aqueous workup followed by purification by C18 reverse phase EZ-Prep using formic acid as the additive gave 4-chloro-N-[(3R)-1-methyl-3-piperidyl]phthalazin-1-amine (60 mg, 21.6% Yield) as a yellow solid. MS m/z 277.0, 279.0 [M+H]⁺. ¹H NMR (400 MHz, DMSO-d6) δ 8.47-8.44 (d, 1H), 8.08-8.04 (m, 1H), 8.02-7.96 (m, 2H), 7.27 (d, J=7.6 Hz, 1H), 4.35-4.28 (m, 1H), 3.05 (d, J=8.0 Hz, 1H), 2.72 (d, J=10.8 Hz, 1H), 2.22 (s, 3H), 1.97-1.94 (m, 3H), 1.77-1.72 (m, 1H), 1.65-1.54 (m, 1H), 1.49-1.39 (m, 1H).

The compounds below were prepared according to the procedure of Example Intermediate 19a by substituting the appropriate starting materials (commercially available), reagents and reaction conditions.

| Structure | Spectral Data |
|---|---|
| 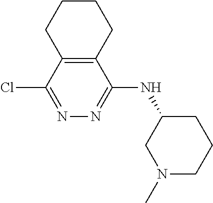 [SMILES: CN1CCC[C@@H](Nc2nnc(Cl)c3c2CCCC3)C1] | MS m/z 281.2, 283.2 [M + H]⁺. |
| 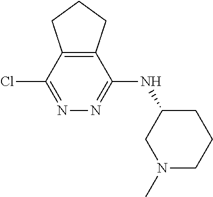 [SMILES: CN1CCC[C@@H](Nc2nnc(Cl)c3c2CCC3)C1] | ¹H NMR (400 MHz, DMSO-d6) δ 6.17 (d, J = 8.0 Hz, 1 H), 4.16-4.08 (m, 1H), 2.92 (d, J = 7.6 Hz, 1 H), 2.84 (t, J = 7.6 Hz, 2 H), 2.77 (t, J = 7.6 Hz, 2 H), 2.65 (d, J = 10.8 Hz, 1 H), 2.18 (s, 3H), 2.10-2.02 (m, 2H), 1.93-1.84 (m, 3H), 1.71-1.66 (m, 1 H), 1.59-1.54 (m, 1 H), 1.37-1.27 (m, 1 H) |
| 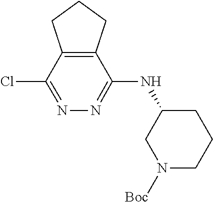 [SMILES: CC(C)(C)OC(=O)N1CCC[C@@H](Nc2nnc(Cl)c3c2CCC3)C1] | MS m/z 353 [M + H]⁺; ¹H NMR (400 MHz, CDCl₃) δ 4.30-4.26 (m, 1H), 3.64-3.55 (m, 3H), 3.30-3.27 (s, 1H), 2.97 (t, J = 7.6 Hz, 2H), 2.78 (t, J = 6.4 Hz, 2H), 2.24-2.16 (m, 2H), 1.92-1.90 (m ,2H), 1.72-1.69 (m, 1H), 1.57-1.55 (m, 1H), 1.42 (s, 9H). |
| 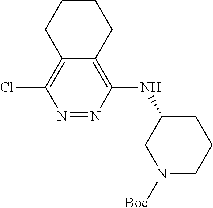 [SMILES: CC(C)(C)OC(=O)N1CCC[C@@H](Nc2nnc(Cl)c3c2CCCC3)C1] | MS m/z 367.2 [M + H]⁺. |
| 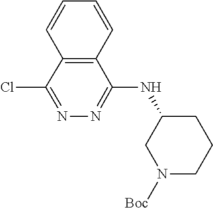 [SMILES: CC(C)(C)OC(=O)N1CCC[C@@H](Nc2nnc(Cl)c3ccccc23)C1] | MS m/z 363.1, 365.0 [M + H]⁺. |

### Intermediate 19b: tert-Butyl (R)-3-((4-chlorophthalazin-1-yl)oxy)piperidine-1-carboxylate

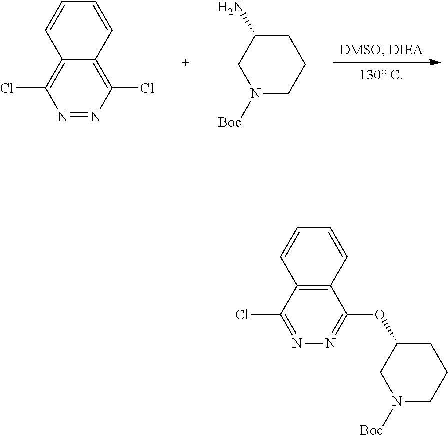 [SMILES: Clc1nnc(Cl)c2ccccc12 ; CC(C)(C)OC(=O)N1CCC[C@@H](N)C1 ; CC(C)(C)OC(=O)N1CCC[C@@H](Oc2nnc(Cl)c3ccccc23)C1]

To a solution of 1,4-dichlorophthalazine (200 mg, 1.0048 mmol) in N,N-dimethylformamide (2 mL) was added tert-butyl (3R)-3-hydroxypiperidine-1-carboxylate (1.2 equiv., 1.21 mmol) and sodium tert-butoxide (1 mL, 2 M in THF). The resulted mixture was bubbled with argon and then was added (2-Dicyclohexylphosphino-2′,4′,6′-triisopropyl-1,1′-biphenyl)[2-(2′-amino-1,1′-biphenyl)]palladium(II) methanesulfonate (0.05 equiv., 0.050 mmol). The reaction mixture was kept bubbling argon for another 5 min. The reaction vessel was sealed, the reaction was heated to 100° C. for 5 h. UPLC showed completion of the reaction, the reaction mixture was filtered through a pad of Celite. The filtrate was worked up and concentrated. The residue was purified over silica with methanol and dichloromethane (0 to 30% gradient) to give tert-butyl (3R)-3-(4-chlorophthalazin-1-yl)oxypiperidine-1-carboxylate (300 mg, 82% Yield). MS m/z 364.0 [M+H]⁺.

### Intermediate 20a: (R)-1-Chloro-N-(1-methylpiperidin-3-yl)pyrido[3,4-d]pyridazin-4-amine and

### Intermediate 20b: (R)-4-Chloro-N-(1-methylpiperidin-3-yl)pyrido[3,4-d]pyridazin-1-amine

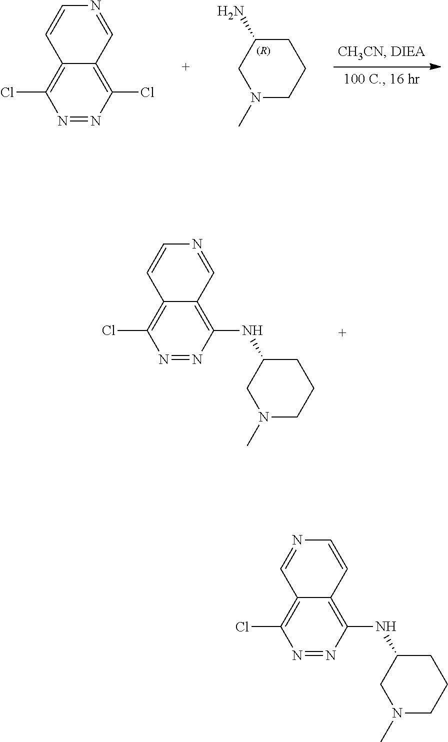

To the solution of 1,4-dichloropyrido[3,4-d]pyridazine (28 g, 140 mmol) in acetonitrile (824 mL, 0.17M) was added (3R)-1-methylpiperidin-3-amine dihydrochloride (39.3 g, 210 mmol, 1.5 eq) and DIPEA (54.3 g, 420 mmol, 3 eq). Then the mixture was stirred at 100° C. for 16 h under N₂. After the reaction, precipitate was filtered under reduced pressure to give a light-yellow solid salt (20 g) and the filtrate was concentrated in vacuum to give a brown residue. The light-yellow solid salt was solved in aqueous saturated sodium carbonate solution and the pH value was adjusted to 9 by aqueous saturated sodium carbonate solution. This aqueous solution was extracted with dichloromethane (150 ml×2). The combined organics were dried over sodium sulfate, concentrated and purified by silica gel column chromatography using FlashColum (8% MeOH/DCM) to get (R)-1-chloro-N-(1-methylpiperidin-3-yl)pyrido[3,4-d]pyridazin-4-amine (13 g, 33% Yield) as a light yellow solid. MS m/z 278 [M+H]⁺. ¹H NMR (400 MHz, DMSO-d6) δ 9.80 (s, 1H), 9.05 (d, J=5.6 Hz, 1H), 7.85 (d, J=5.6 Hz, 1H), 7.76 (d, J=7.5 Hz, 1H), 4.55-4.24 (m, 1H), 3.05 (d, J=7.4 Hz, 1H), 2.72 (d, J=10.9 Hz, 1H), 2.21 (s, 3H), 2.04-1.82 (m, 3H), 1.82-1.69 (m, 1H), 1.67-1.51 (m, 1H), 1.43 (m, 1H).

The brown residue was dissolved in DCM (300 ml), washed with water (100 ml×3), dried over sodium sulfate, concentrated and purified by silica gel column chromatography using FlashColum (8% MeOH/DCM) to give a mixture of region-isomers which were further purified on SFC to provide another regio-isomer, (R)-4-chloro-N-(1-methylpiperidin-3-yl)pyrido[3,4-d]pyridazin-1-amine, as clean product. MS m/z 278 [M+H]⁺. ¹H NMR (400 MHz, DMSO-d6) δ 9.40 (s, 1H), 9.09 (d, J=8.0 Hz, 1H), 8.35 (d, J=8.0 Hz, 1H), 7.57 (d, J=8.0 Hz, 1H), 4.34-4.29 (m, 1H), 3.03 (d, J=7.4 Hz, 1H), 2.71 (d, J=10.9 Hz, 1H), 2.21 (s, 3H), 2.0-1.9 (m, 3H), 1.74-1.73 (m, 1H), 1.60-1.57 (m, 1H), 1.44-1.40 (m, 1H).

### Intermediate 20c: (R)-(1-(2-((tert-Butyldimethylsilyl)oxy)ethyl)piperidin-3-yl)-1-chloropyrido[3,4-d]pyridazin-4-amine and

### Intermediate 20d: (R)—N-(1-(2-((tert-Butyldimethylsilyl)oxy)ethyl)piperidin-3-yl)-4-chloropyrido[3,4-d]pyridazin-1-amine

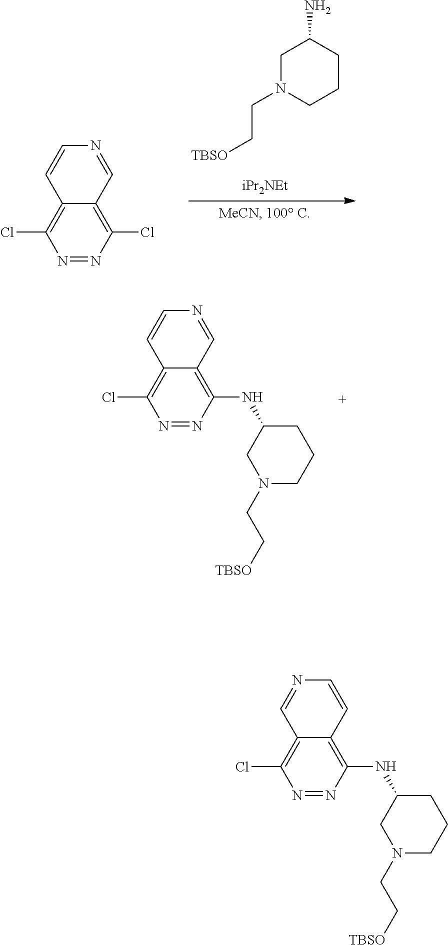 [SMILES: "iPr2NEt" ; Clc1nnc(Cl)c2cnccc12 ; CC(C)(C)[Si](C)(C)OCCN1CCC[C@@H](N)C1 ; CC(C)(C)[Si](C)(C)OCCN1CCC[C@@H](Nc2nnc(Cl)c3ccncc23)C1 ; CC(C)(C)[Si](C)(C)OCCN1CCC[C@@H](Nc2nnc(Cl)c3cnccc23)C1]

To a solution of 1,4-dichloropyrido[3,4-d]pyridazine (1.75 g, 8.75 mmol) and (3R)-1-[2-[tert-butyl(dimethyl)silyl]oxyethyl]piperidin-3-amine (Intermediate 15a, 2.49 g, 9.62 mmol) in MeCN (48 mL) was added iPr₂NEt (4.6 mL, 26.2 mmol). The solution was sparged with Ar for 10 min and then heated to 100° C. for 6 h. The reaction was concentrated and purified by chromatography on SiO₂ (MeOH:DCM, 0 to 10%) to give an orange gum containing a −3:1 mixture of compound (3R)-1-[2-[tert-butyl(dimethyl)silyl]-oxyethyl]piperidin-3-amine and its regioisomer (2.89 g, 78%), which were separated by SFC.

(3R)-1-[2-[tert-Butyl(dimethyl)silyl]oxyethyl]piperidin-3-amine: MS m/z 422.0 [M+H]⁺; ¹H NMR (400 MHz, DMSO-d₆) δ 9.79 (s, 1H), 9.05 (d, J=5.6 Hz, 1H), 7.86 (d, J=5.5 Hz, 1H), 7.72 (d, J=7.5 Hz, 1H), 4.40-4.27 (m, 1H), 3.69 (t, J=6.1 Hz, 2H), 3.20-3.11 (m, 1H), 2.89-2.78 (m, 1H), 2.48-2.41 (m, 2H), 2.09-1.95 (m, 3H), 1.80-1.66 (m, 1H), 1.64-1.50 (m, 1H), 1.49-1.38 (m, 1H), 0.82 (s, 9H), 0.03 (s, 6H).

(R)—N-(1-(2-((tert-Butyldimethylsilyl)oxy)ethyl)piperidin-3-yl)-4-chloropyrido[3,4-d]pyridazin-1-amine: MS m/z 422.0 [M+H]⁺; ¹H NMR (400 MHz, DMSO-d₆) δ 9.40 (s, 1H), 9.09 (d, J=5.6 Hz, 1H), 8.33 (d, J=5.6 Hz, 1H), 7.53 (d, J=7.6 Hz, 1H), 4.36-4.18 (m, 1H), 3.69 (t, J=6.1 Hz, 2H), 3.19-3.07 (m, 1H), 2.84 (d, J=11.3 Hz, 1H), 2.49-2.43 (m, 2H), 2.10 -1.91 (m, 3H), 1.79-1.65 (m, 1H), 1.62-1.36 (m, 2H), 0.82 (s, 9H), 0.03 (s, 6H).

### Intermediate 20e: tert-Butyl (R)-3-((1-chloropyrido[3,4-d]pyridazin-4-yl)amino)piperidine-1-carboxylate and

### Intermediate 20f: tert-Butyl (R)-3-((4-chloropyrido[3,4-d]pyridazin-1-yl)amino)piperidine-1-carboxylate

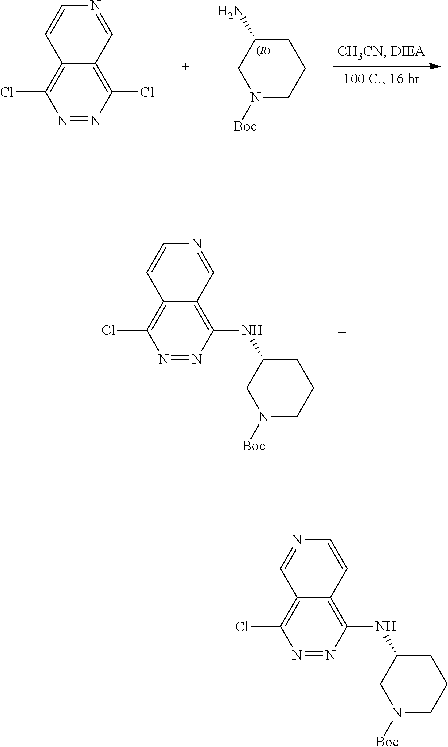

The title compounds were prepared in analogous manner according to the procedure of Intermediate 20c and Intermediate 20d, using tert-butyl (R)-3-aminopiperidine-1-carboxylate in place of (3R)-1-[2-[tert-butyl(dimethyl)silyl]oxyethyl]piperidin-3-amine.

tert-Butyl (R)-3-((1-chloropyrido[3,4-d]pyridazin-4-yl)amino)piperidine-1-carboxylate. ¹H NMR (acetone-d6) δ: 9.49 (s, 1H), 9.06 (d, J=5.5 Hz, 1H), 8.14 (br s, 1H), 6.88 (br s, 1H), 4.30-4.45 (m, 1H), 4.00-4.25 (m, 1H), 3.72-3.85 (m, 1H), 3.00-3.40 (m, 2H), 2.15-2.25 (m, 1H), 1.70-1.93 (m, 2H), 1.55-1.67 (m, 1H), 1.30-1.50 (br s, 9H).

tert-Butyl (R)-3-((4-chloropyrido[3,4-d]pyridazin-1-yl)amino)piperidine-1-carboxylate. ¹H NMR (acetone-d6) δ: 9.69 (s, 1H), 9.08 (d, J=5.5 Hz, 1H), 7.93 (d, J=5.5 Hz, 1H), 7.10 (br s, 1H), 4.30-4.45 (m, 1H), 4.03-4.25 (m, 1H), 3.79-3.90 (m, 1H), 3.00-3.40 (m, 2H), 2.15-2.25 (m, 1H), 1.70-1.97 (m, 2H), 1.50-1.64 (m, 1H), 1.30-1.50 (br s, 9H).

### Intermediate 21a: (R)-5-Chloro-N-(1-methylpiperidin-3-yl)pyrido[2,3-d]pyridazin-8-amine and

### Intermediate 21b: (R)-8-Chloro-N-(1-methylpiperidin-3-yl)pyrido[2,3-d]pyridazin-5-amine

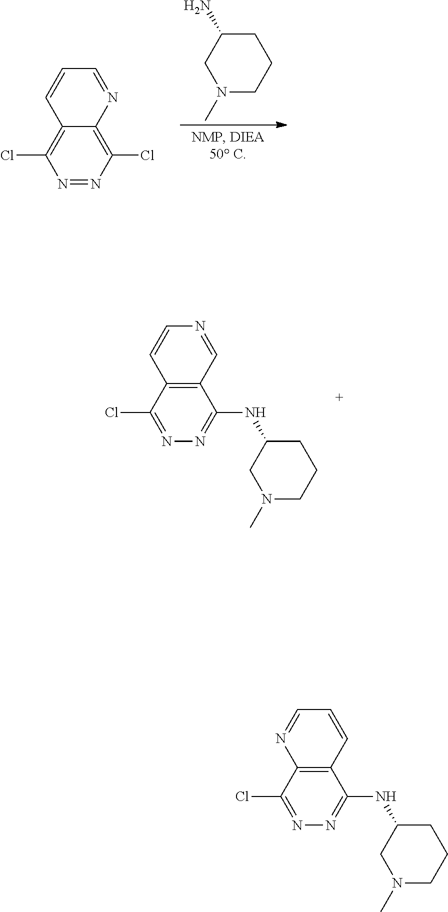

A solution of (3R)-1-methylpiperidin-3-amine (1.14 g, 10 mmol, 2 eq) in NMP (2 mL) was added to a solution of 5,8-dichloropyrido[2,3-d]pyridazine (1.00 g, 5.0 mmol, 1 eq) in NMP (18 mL) followed by the addition of N,N-diisopropylethylamine (2.0 mL, 11.5 mmol, 2.3 eq) and was stirred at 50° C. for 1 day. After completion, the reaction was diluted with EtOAc and NaHCO₃(sat. aq.). The product was extracted several times with EtOAc (100 mL×5) from 1M K₂CO₃ (200 mL) and the combined organic extracts were washed with a small amount of 1M K₂CO₃ (2×25 mL), washed with 25 mL brine and were dried over Na₂SO₃ and concentrated in vacuo. The crude residue was purified by silica gel column chromatography eluting with 1:99 to 25:75 MeOH:DCM to afford (R)-5-chloro-N-(1-methylpiperidin-3-yl)pyrido[2,3-d]pyridazin-8-amine (497 mg, 36%) and (R)-8-chloro-N-(1-methylpiperidin-3-yl)pyrido[2,3-d]pyridazin-5-amine (643 mg, 46%), respectively.

(R)-5-Chloro-N-(1-methylpiperidin-3-yl)pyrido[2,3-d]pyridazin-8-amine. MS m/z 278.3 [M+H]⁺. ¹H NMR (400 MHz, DMSO-d6) δ 9.20 (d, J=4.5 Hz, 1H), 8.47 (d, J=8.6 Hz, 1H), 8.08-8.01 (m, 1H), 7.41 (d, J=8.3 Hz, 1H), 4.40-4.25 (m, 1H), 2.73-2.59 (m, 1H), 2.42-2.25 (m, 3H), 2.21 (s, 3H), 1.81-1.62 (m, 3H), 1.59-1.48 (m, 1H).

(R)-8-Chloro-N-(1-methylpiperidin-3-yl)pyrido[2,3-d]pyridazin-5-amine. MS m/z 278.3 [M+H]⁺. ¹H NMR (400 MHz, DMSO-d6) δ 9.21 (d, J=4.4 Hz, 1H), 8.89 (d, J=1.3 Hz, 1H), 8.03-7.92 (m, 1H), 7.54-7.39 (m, 1H), 4.37-4.21 (m, 1H), 3.07-2.95 (m, 1H), 2.77-2.60 (m, 1H), 2.19 (s, 3H), 2.03-1.82 (m, 3H), 1.80-1.68 (m, 1H), 1.64-1.50 (m, 1H), 1.49-1.32 (m, 1H).

### Intermediate 22: tert-Butyl (R)-3-((4-chloro-6,7,8,9-tetrahydro-5H-cyclohepta[d]pyridazin-1-yl)amino)piperidine-1-carboxylate

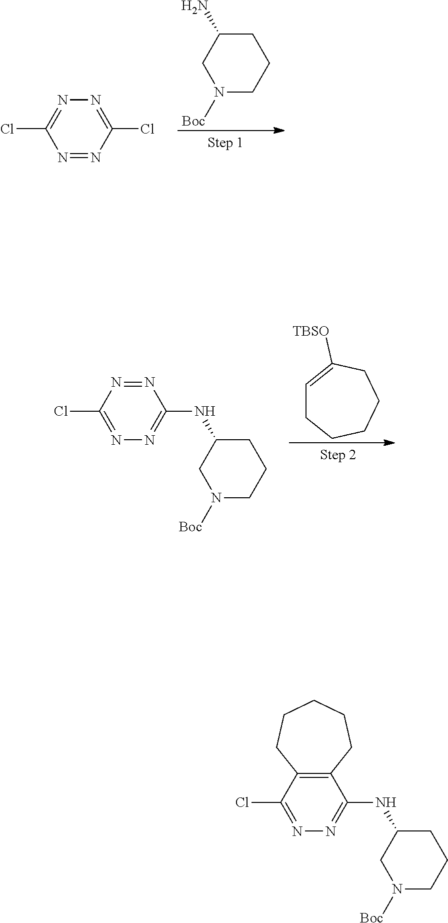 [SMILES: Clc1nnc(Cl)nn1 ; CC(C)(C)OC(=O)N1CCC[C@@H](N)C1 ; CC(C)(C)OC(=O)N1CCC[C@@H](Nc2nnc(Cl)nn2)C1 ; CC(C)(C)[Si](C)(C)OC1=CCCCCC1 ; CC(C)(C)OC(=O)N1CCC[C@@H](Nc2nnc(Cl)c3c2CCCCC3)C1]

### Step 1: tert-Butyl (R)-3-((6-chloro-1,2,4,5-tetrazin-3-yl)amino)piperidine-1-carboxylate

3,6-Dichloro-1,2,4,5-tetrazine (100 mg, 0.66 mmol) and tert-butyl (R)-3-aminopiperidine-1-carboxylate (138 mg, 0.74 mmol) were dissolved in DCM (1 mL) at 0° C. DIPEA (150 μL, 0.81 mmol) was added. This was stirred at 0° C. for 15 minutes. The reaction mixture was loaded directly on a silica column. Purification by silica chromatography (0-50% EtOAc in DCM) yielded tert-butyl (R)-3-((6-chloro-1,2,4,5-tetrazin-3-yl)amino)piperidine-1-carboxylate (200 mg, 97%) as a red solid. ¹H NMR (acetone-d6) δ: 7.80 (br s, 1H), 3.95-4.10 (m, 2H), 3.65-3.79 (m, 1H), 3.05-3.40 (m, 2H), 2.12-2.19 (m, 1H), 1.73-1.90 (m, 2H), 1.50-1.63 (m, 1H), 1.41 (s, 9H).

### Step 2. tert-Butyl (R)-3-((4-chloro-6,7,8,9-tetrahydro-5H-cyclohepta[d]pyridazin-1-yl)amino)piperidine-1-carboxylate

Tert-butyl (R)-3-((6-chloro-1,2,4,5-tetrazin-3-yl)amino)piperidine-1-carboxylate (100 mg, 0.32 mmol), (cyclohept-1-en-1-yloxy)trimethylsilane (68 mg, 0.37 mmol) and toluene (0.3 mL) were heated at 120° C. in a sealed tube under argon for 15 hours. The reaction mixture was loaded directly on a silica column. Purification by silica chromatography (0-50% EtOAc in DCM) yielded (R)-4-chloro-N-(1-methylpiperidin-3-yl)-6,7,8,9-tetrahydro-5H-cyclohepta[d]pyridazin-1-amine (47 mg, 39%) as a reddish semisolid. MS m/z 380.6 [M+H]⁺; ¹H NMR (acetone-d6) δ: 5.55 (br s, 1H), 4.10-4.20 (br s, 1H), 4.04 (d, J=14 Hz, 1H), 3.66-3.75 (m, 1H), 3.00-3.20 (m, 2H), 2.97 (t, J=5.6, 2H), 2.76 (t, J=5.6, 2H), 1.91 (t, J=5.6, 2H), 1.50-1.80 (m, 8H), 1.43 (s, 9H).

### Intermediate 23: tert-Butyl (R)-3-((4-chloro-5,5-dimethyl-6,7-dihydro-5H-cyclopenta[d]pyridazin-1-yl)amino)piperidine-1-carboxylate

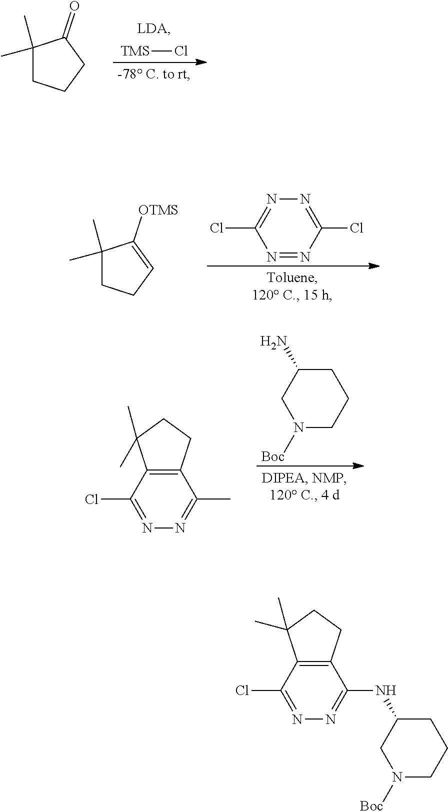

### Step 1: ((5,5-Dimethylcyclopent-1-en-1-yl)oxy)trimethylsilane

LDA (2M in THF, 4.8 L, 9.6 mmol) was cooled to −78° C. A solution of 2,2-dimethylcyclopentan-1-one (1 g, 8.9 mmol) was added dropwise. This was stirred at −78° C. for 30 minutes. Chlorotrimethylsilane (2 mL, 15.7 mmol) was then added dropwise. The mixture was warmed to room temperature over 90 minutes, then quenched with saturated aqueous NaHCO₃, and diluted with pentane. The bilayer was filtered through celite. The organic layer was separated, dried over MgSO₄, filtered, and concentrated under vacuum. Purification by silica gel chromatography (hexanes) yielded ((5,5-dimethylcyclopent-1-en-1-yl)oxy)trimethylsilane at 90% purity. (923 mg, 50%). ¹H NMR (acetone-d6) δ: 4.49 (s, 1H), 2.15 (t, J=7.5 Hz, 2H), 1.69 (t, J=7.5 Hz, 2H), 1.00 (s, 6H), 0.21 (s, 9H).

### Step 2. 1,4-Dichloro-5,5-dimethyl-6,7-dihydro-5H-cyclopenta[d]pyridazine

((5,5-Dimethylcyclopent-1-en-1-yl)oxy)trimethylsilane (750 mg, 4.07 mmol), toluene (4 mL), and 3,6-dichloro-1,2,4,5-tetrazine (610 mg, 4.04 mmol) were heated under argon at 120° C. for 15 hours. Toluene was then removed by nitrogen stream. Purification by silica gel chromatography (0-10% EtOAc in DCM), followed by hexane trituration, yielded 1,4-dichloro-5,5-dimethyl-6,7-dihydro-5H-cyclopenta[d]pyridazine (360 mg, 41%) as a light pink solid. ¹H NMR (acetone-d6) δ: 3.09 (t, J=7.5 Hz, 2H), 2.14 (t, J=7.5 Hz, 2H), 1.47 (s, 6H).

### Step 3: tert-Butyl (R)-3-((4-chloro-5,5-dimethyl-6,7-dihydro-5H-cyclopenta[d]pyridazin-1-yl)amino)piperidine-1-carboxylate

1,4-Dichloro-5,5-dimethyl-6,7-dihydro-5H-cyclopenta[d]pyridazine (500 mg, 2.3 mmol), (R)-1-Boc-3-aminopiperidine (520 mg, 2.5 mmol), DIPEA (1.35 mL, 7.74 mmol) and NMP (5.4 mL) were heated at 120° C. for 4 days, then partitioned between H₂O and EtOAc. The organic layer was back-washed with H₂O, dried over MgSO₄, filtered, and concentrated under vacuum. Purification by silica gel chromatography (0-100% EtOAc in DCM), followed by hexane trituration, yielded tert-butyl (R)-3-((4-chloro-5,5-dimethyl-6,7-dihydro-5H-cyclopenta[d]pyridazin-1-yl)amino)piperidine-1-carboxylate (166 mg, 19%) as a white solid, which was applied to the next step without further purification.

### Intermediate 25: tert-Butyl (3R)-3-((4-chloro-5-methyl-5,6,7,8-tetrahydrophthalazin-1-yl)amino)piperidine-1-carboxylate

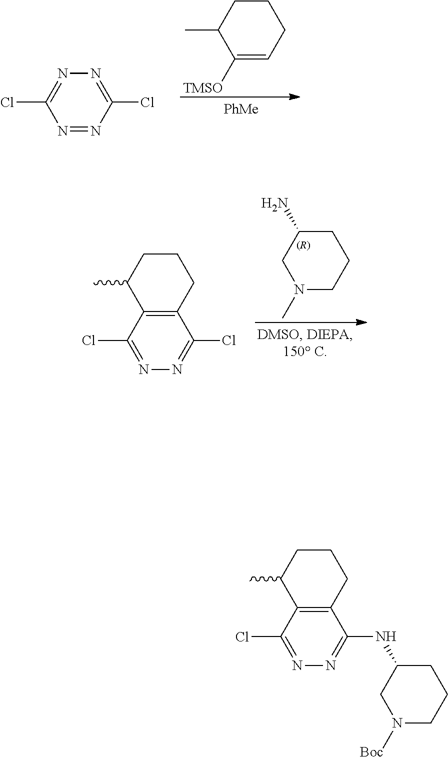

### Step 1: 1,4-Dichloro-5-methyl-5,6,7,8-tetrahydrophthalazine

3,6-Dichloro-1,2,4,5-tetrazine (200 mg, 1.32 mmol, 1.0 eq.) and trimethyl((6-methylcyclohex-1-en-1-yl)oxy)silane (317 mg, 1.3 eq) were mixed in anhydrous toluene (1.0 mL) under argon. The mixture was heated to 120° C. for 2 hr in a sealed tube. After cooling and concentrated, the crude material was purified by flash column chromatography on silica gel eluting with 0-30% EtOAc in hexane to provide 1,4-dichloro-5-methyl-5,6,7,8-tetrahydrophthalazine (89 mg, 30% yield). ¹H NMR (acetone-d6) δ: 2.97 (dt, J=6.91, 3.4 Hz, 1H), 2.65-2.74 (m, 1H), 2.35-2.54 (m, 1H), 1.72-1.81 (m, 2H), 1.62-1.69 (m, 2H), 1.09-1.13 (d, J=6.91, 3H).

### Step 2. tert-Butyl (3R)-3-((4-chloro-5-methyl-5,6,7,8-tetrahydrophthalazin-1-yl)amino) piperidine-1-carboxylate

1,4-Dichloro-5-methyl-5,6,7,8-tetrahydrophthalazine (56 mg, 0.26 mmol), (R)-1-Boc-3-aminopiperidine (56 mg, 0.26 mmol), DIPEA (68 mg, 0.52 mmol) and NMP (0.5 mL) were heated at 130° C. for 2 days, then partitioned between H₂O and EtOAc. The organic layer was back-washed with H₂O, dried over MgSO₄, filtered, and concentrated under vacuum. Purification by silica gel chromatography (0-100% EtOAc in DCM), followed by hexane trituration, yielded tert-butyl (3R)-3-((4-chloro-5-methyl-5,6,7,8-tetrahydrophthalazin-1-yl)amino) piperidine-1-carboxylate (25 mg, 25%), which was applied to the next step without further purification. MS m/z 381.1 [M+H]⁺.

### Intermediate 26: (R)-5-Chloro-1-methyl-N-(1-methylpiperidin-3-yl)-1,2,3,4-tetrahydropyrido[2,3-d]pyridazin-8-amine

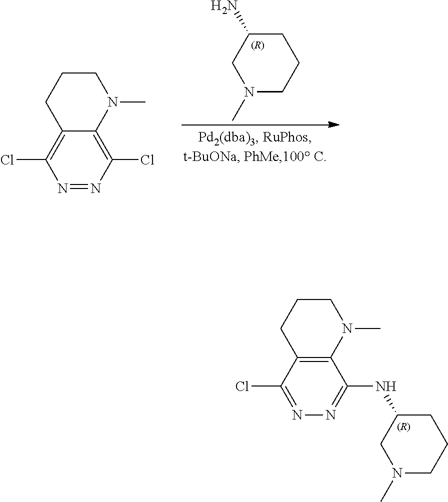

A solution of 5,8-dichloro-1-methyl-3,4-dihydro-2H-pyrido[2,3-d]pyridazine (prepared according to WO2020239076A1, 200 mg, 0.92 mmol), (3R)-1-methylpiperidin-3-amine (127 mg, 1.1 mmol), RuPhos (47 mg, 0.1 mmol), tris(dibenzylideneacetone)dipalladium (88 mg, 0.1 mmol) and sodium tert-butoxide (187 mg, 2 mmol) in toluene (5 mL) was stirred at 100° C. for 12 hours under N₂ atmosphere. After the reaction, solvent was removed and the crude residue was purified over silica gel using 30% EA/PE to give (R)-5-chloro-1-methyl-N-(1-methylpiperidin-3-yl)-1,2,3,4-tetrahydropyrido[2,3-d]pyridazin-8-amine (70 mg, 26% Yield). MS m/z 296.2 [M+H]⁺.

### Intermediate 27: tert-Butyl (R,Z)-3-((hydrazineyl(methylthio)methylene)amino)piperidine-1-carboxylate

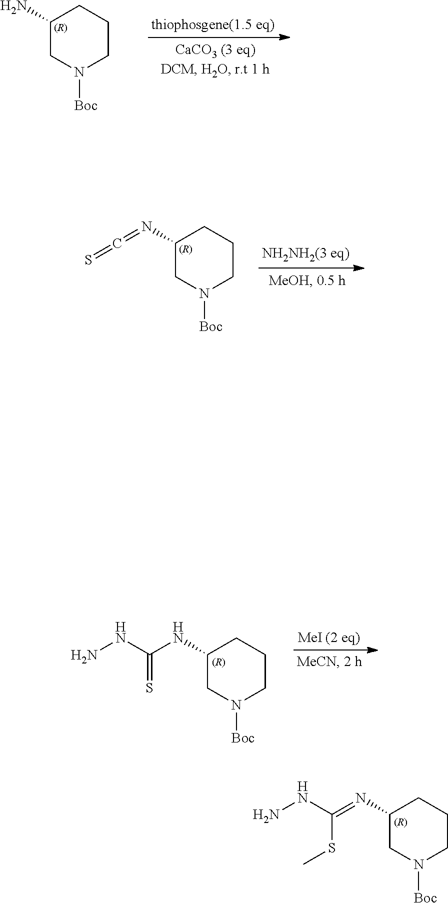

### Step 1. tert-Butyl (R)-3-isothiocyanatopiperidine-1-carboxylate

To a solution of CaCO₃ (7.5 g, 75 mmol) in DCM (100 mL) and water (50 mL) was added slowly tert-butyl (3R)-3-aminopiperidine-1-carboxylate (5.0 g, 25 mmol) and thiophosgene (4.3 g, 37 mmol) at 0° C. under N₂. Then the reaction mixture was stirred at room temperature for 1 h. The mixture was filtered and the filtrate was extracted with DCM (2×50 mL), dried over anhydrous Na₂SO₄, and evaporated in vacuum. The crude product was purified on silica gel eluted with PE/EA=8:1 to give the compound tert-butyl (R)-3-isothiocyanatopiperidine-1-carboxylate (6.0 g, 99.2% Yield) as a pale yellow oil. MS m/z 143 [M-Boc+H]⁺.

### Step 2. tert-Butyl (R)-3-(hydrazinecarbothioamido)piperidine-1-carboxylate

To a solution of tert-butyl (R)-3-isothiocyanatopiperidine-1-carboxylate (6.0 g, 25 mmol) in methanol (50 mL) was added hydrazine in water (3.6 mL, 93 mmol, 80%) under N₂, then the reaction mixture was stirred at 25° C. for one hour, evaporated in vacuum, then brine (80 mL) was added and extracted with DCM (2×80 mL), dried over Na₂SO₄, evaporated in vacuum to give compound tert-butyl (R)-3-(hydrazinecarbothioamido)piperidine-1-carboxylate (6.0 g, 99.2% Yield) as a pale yellow oil, which could be used next step without further purification. MS m/z 275.1 [M+H]⁺.

### Step 3. tert-Butyl (R)-3-isothiocyanatopiperidine-1-carboxylate

To a solution of tert-butyl (3R)-3-(aminocarbamothioylamino)piperidine-1-carboxylate (6.0 g, 21.9 mmol) in ACN (60 mL) was added MeI (6.2 g, 43.8 mmol) at 0° C. under N₂. The reaction mixture was stirred at 25° C. for 2 h. The reaction mixture was concentrated under reduced pressure, diluted with DCM (50 mL) and washed with saturated NaHCO₃ (2×40 mL), dried over anhydrous Na₂SO₄, and evaporated in vacuo. The crude product was purified on silica gel eluted with (PE/EA=2:1) to give the compound tert-butyl (R)-3-isothiocyanatopiperidine-1-carboxylate (1.9 g, 30.2% Yield) as a brown oil. MS m/z 289.0 [M+H]⁺. ¹H NMR (CHLOROFORM-d) δ: 10.21 (br s, 1H), 3.70-3.78 (m, 2H), 3.47 (br s, 1H), 3.38 (br s, 2H), 2.97 (s, 3H), 1.95-2.04 (m, 1H), 1.73-1.85 (m, 2H), 1.56-1.66 (m, 1H), 1.47 (s, 9H)

### Example 1

### Preparation of Compound I-1

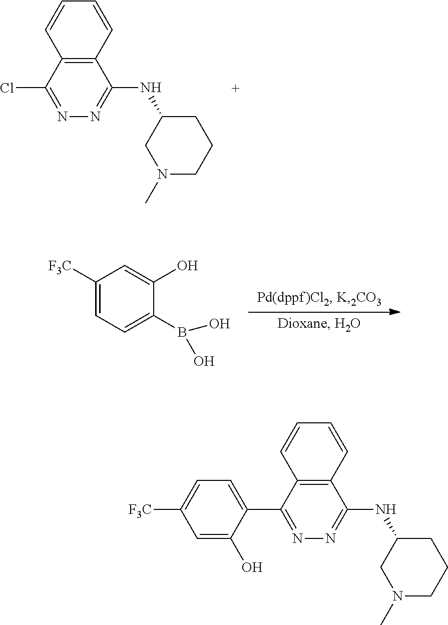 [SMILES: CN1CCC[C@@H](Nc2nnc(Cl)c3ccccc23)C1 ; OB(O)c1ccc(C(F)(F)F)cc1O ; CN1CCC[C@@H](Nc2nnc(-c3ccc(C(F)(F)F)cc3O)c3ccccc23)C1]

To a solution of 4-chloro-N-[(3R)-1-methyl-3-piperidyl]phthalazin-1-amine (Intermediate 19a, 55 mg, 0.20 mmol) in 1,4-dioxane (4 mL) was added [2-hydroxy-4-(trifluoromethyl)phenyl]boronic acid (1.2 eq., 0.24 mmol), and potassium carbonate in water (2 M water solution), and bubbled nitrogen through for 5 minutes. [1,1′-bis(diphenylphosphino)ferrocene]dichloropalladium(II) (0.1 eq., 0.020 mmol) was then added and the reaction mixture was kept bubbling nitrogen for another 5 min before. and then the reaction vessel was sealed. The reaction was heated to 100° C. for 3 h. The reaction mixture was purified by C18 reverse phase EZ-Prep using formic acid as the additive to give 2-[4-[[(3R)-1-methyl-3-piperidyl]amino]phthalazin-1-yl]-5-(trifluoromethyl)phenol (35 mg, 44% yield) as formic acid salt. MS m/z 403.0 [M+H]⁺, ¹H NMR (METHANOL-d4) δ: 8.52 (s, 1H, formic acid proton), 8.32 (br d, J=8.1 Hz, 1H), 7.87-7.95 (m, 1H), 7.79-7.87 (m, 1H), 7.62 (br d, J=8.1 Hz, 1H), 7.52 (br d, J=7.9 Hz, 1H), 7.28-7.36 (m, 1H), 7.28 (s, 1H), 4.60-4.70 (m, 1H), 3.54-3.69 (m, 1H), 3.13-3.24 (m, 1H), 2.76-2.92 (m, 2H), 2.76 (s, 3H), 2.03-2.25 (m, 2H), 1.77-1.98 (m, 2H). NH and OH not observed.

### Example 2

### Preparation of Compound I-156

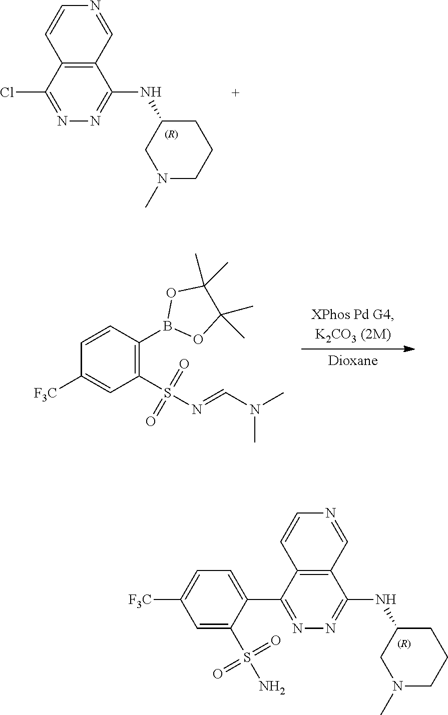

A solution of 1-chloro-N-[(3R)-1-methyl-3-piperidyl]pyrido[3,4-d]pyridazin-4-amine (Intermediate 20a, 145 mg, 0.5 mmol), (E)-N,N-dimethyl-N′-((2-(4,4,5,5-tetramethyl-1,3,2-dioxaborolan-2-yl)-5-(trifluoromethyl)phenyl)sulfonyl)formimidamide (Intermediate 10, 300 mg, 0.75 mmol), XPhos Pd G4 (43 mg, 0.05 mmol) and potassium carbonate (138 mg, 1 mmol) in 1,4-dioxane (2 mL) and water (0.5 mL) was stirred at 100° C. under N₂ for 2 h. After that, some starting material remains, so another portion of XPhos Pd G4 (43 mg, 0.05 mmol) was added in and the solution was stirred for another 2 h at 100° C. under N₂, monitored by LCMS.

After that, the mixture was washed with water and brine, dried over Na₂SO₄, concentrated and purified by Prep-HPLC to give (R)-2-(4-((1-methylpiperidin-3-yl)amino)pyrido[3,4-d]pyridazin-1-yl)-5-(trifluoromethyl)benzene-sulfonamide (35 mg, 20% yield) as yellow solid. MS m/z 467.0 [M+H]⁺. ¹H NMR (400 MHz, CD₃OD) δ 9.49 (s, 1H), 8.93 (d, J=5.4 Hz, 1H), 8.32 (s, 1H, formic acid), 8.30-8.23 (m, 2H), 7.90-7.85 (m, 1H), 7.76-7.70 (m, 1H), 4.20-4.10 (m, 1H), 3.25-3.15 (m, 1H), 2.92-2.79 (m, 1H), 2.45 (s, 3H), 2.41-2.26 (m, 2H), 2.08-1.99 (m, 1H), 1.98-1.88 (m, 1H), 1.82-1.69 (m, 1H), 1.65-1.53 (m, 1H).

The compounds below were prepared according to the procedure of Examples 1 or 2 by substituting the appropriate starting materials, reagents and reaction conditions.

| Compound | Spectral Data |
|---|---|
| I-2 | MS m/z 407.2 [M + H]⁺; ¹H NMR (METHANOL-d₄) δ: 8.36 (s, 2H, formic |
| | acid), 7.37 (d, J = 7.8 Hz, 1H), 7.26 (d, J = 8.0 Hz, 1H), 7.20 (s, 1H), 4.51-4.65 |
| | (m, 1H), 3.63-3.85 (m, 1H), 3.35-3.42 (m, 1H), 2.95-3.12 (m, 2H), 2.87 (s, 3H), |
| | 2.50 (dt, J = 17.7, 6.2 Hz, 4H), 2.04-2.23 (m, 2H), 1.86-1.98 (m, 3H), 1.72-1.86 |
| | (m, 3H) |
| I-3 | MS m/z 393.3 [M + H]⁺; ¹H NMR (CD₃OD) δ: 8.41 (br s, 2H, formic acid |
| | proton), 7.72 (br d, J = 7.8 Hz, 1H), 7.16-7.28 (m, 2H), 4.53 (br s, 1H), 3.64-3.82 |
| | (m, 1H), 3.35-3.46 (m, 1H), 3.10-3.25 (m, 2H), 2.92-3.07 (m, 3H), 2.78-2.89 |
| | (m, 4H), 2.12-2.28 (m, 4H), 1.95 (br d, J = 10.4 Hz, 1H), 1.81 (br s, 1H). |
| I-4 | MS m/z 409.0 [M + H]⁺; ¹H NMR (CD₃OD) δ: 8.35 (s, 1H), 8.05 (br d, J = 5.4 Hz, |
| | 1H), 7.97 (br d, J = 8.0 Hz, 1H), 7.81 (br d, J = 5.4 Hz, 1H), 7.15-7.26 (m, 2H), |
| | 4.46-4.63 (m, 1H), 3.57-3.73 (m, 1H), 2.83-2.97 (m, 1H), 2.74 (s, 3H), 2.57 (s, |
| | 1H), 1.97-2.19 (m, 2H), 1.80-1.94 (m, 1H), 1.67-1.80 (m, 1H), NH and OH not |
| | observed |
| I-5 | MS m/z 404.4 [M + H]⁺; ¹H NMR (400 MHz, CD₃OD) δ 9.09 (dd, J = 4.4, 1.6 |
| | Hz, 1H), 8.01 (dd, J = 8.3, 1.6 Hz, 1H), 7.81 (dd, J = 8.3, 4.4 Hz, 1H), 7.58 (d, |
| | J = 7.9 Hz, 1H), 7.31 (d, J = 7.9 Hz, 1H), 7.25 (s, 1H), 4.55-4.47 (m, 1H), |
| | 3.06 (s, 1H), 2.73-2.55 (m, 1H), 2.48-2.25 (m, 5H), 2.12-1.96 (m, 1H), |
| | 1.94-1.83 (m, 1H), 1.81-1.63 (m, 2H), NH and OH not observed. |
| I-6 | MS m/z 375.1 [M + H]⁺; ¹H NMR (CD₃OD) δ: 8.55 (d, J = 8.3 Hz, 1H), 8.06-8.15 |
| | (m, 1H), 7.96-8.06 (m, 1H), 7.81 (d, J = 8.0 Hz, 1H), 7.62 (d, J = 7.9 Hz, 1H), |
| | 7.29-7.43 (m, 2H), 3.59-3.83 (m, 3H), 3.43-3.58 (m, 1H), 3.35-3.39 (m, 1H), |
| | 2.53-2.64 (m, 1H), 2.41-2.53 (m, 1H), NHs and OH not observed. |
| I-7 | MS m/z 404.3 [M + H]⁺; ¹H NMR (METHANOL-d4) δ: 9.62 (s, 1H), 8.76 (d, |
| | J = 5.5 Hz, 1H), 7.44-7.55 (m, 1H), 7.39 (br d, J = 5.6 Hz, 1H), 7.10-7.32 (m, 2H), |
| | 4.55 (br s, 1H), 2.97-3.17 (m, 1H), 2.64 (br s, 1H), 2.52-2.29 (m, 5H), 2.00 (br |
| | s, 1H), 1.75-1.87 (m, 1H), 1.55-1.75 (m, 2H), NH and OH not observed. |
| I-8 | MS m/z 417.2 [M + H]⁺; ¹H NMR (CD₃OD) δ: 8.20 (br d, J = 8.1 Hz, 1H), 7.83 (t, |
| | J = 7.8 Hz, 1H), 7.70 (br d, J = 7.0 Hz, 1H), 7.41-7.50 (m, 1H), 7.31 (br d, J = 7.6 |
| | Hz, 1H), 7.20 (s, 1H), 4.57-4.77 (m, 1H), 3.80-3.97 (m, 1H), 3.68 (s, 1H), |
| | 3.39-3.56 (m, 1H), 2.97-3.11 (m, 2H), 2.86-2.97 (m, 3H), 2.23 (br s, 1H), 2.18 (s, |
| | 3H), 1.88-2.08 (m, 2H), NH and OH not observed. |
| I-9 | MS m/z 421.0 [M + H]⁺; ¹H NMR (METHANOL-d₄) δ: 8.41-8.47 (m, 1H), 7.70-7.77 |
| | (m, 1H), 7.55 (br d, J = 7.6 Hz, 1H), 7.34 (br d, J = 7.1 Hz, 1H), 7.29 (br s, |
| | 1H), 7.24 (br d, J = 8.9 Hz, 1H), 4.82 (br s, 1H), 4.70 (br s, 1H), 3.77-3.97 (m, |
| | 1H), 3.45 (br s, 1H), 3.13 (br s, 1H), 2.93 (br s, 3H), 2.11-2.33 (m, 2H), |
| | 1.84-2.10 (m, 2H) |
| I-12 | MS m/z 403.2 [M + H]⁺; ¹H NMR (METHANOL-d₄) δ: 8.36 (s, 2H, formic |
| | acid), 7.90-7.95 (m, 1H), 7.85 (br t, J = 7.4 Hz, 1H), 7.64 (br d, J = 8.0 Hz, 1H), |
| | 7.54 (br d, J = 7.8 Hz, 1H), 7.22-7.42 (m, 2H), 4.65-4.83 (m, 1H), 3.83 (br s, |
| | 1H), 3.35-3.46 (m, 1H), 3.03-3.25 (m, 2H), 2.89 (br s, 3H), 2.33-2.13 (m, 2H), |
| | 1.88-2.10 (m, 2H), 1H buried under FA peak, NH and OH not observed. |
| I-23 | MS m/z 407.2 [M + H]⁺; ¹H NMR (CD₃OD) δ: 8.45 (s, 1H, formic acid), 7.29 (br |
| | d, J = 7.6 Hz, 1H), 7.15 (br d, J = 7.9 Hz, 1H), 7.07 (s, 1H), 4.33 (br s, 1H), 3.53 |
| | (br d, J = 10.5 Hz, 1H), 3.13-3.19 (m, 1H), 2.73-2.90 (m, 3H), 2.23-2.52 (m, |
| | 2H), 2.00-2.14 (m, 1H), 1.88-2.00 (m, 1H), 1.79-1.88 (m, 2H), 1.65-1.79 (m, |
| | 3H), 1.57-1.65 (m, 1H), 0.79 (br d, J = 7.0 Hz, 3H). OH and NH not observed. |
| I-33 | MS m/z 335.3 [M + H]⁺; ¹H NMR (400 MHz, DMSO-d6) δ 9.64 (s, 1H), 8.37 (d, |
| | J = 8.1 Hz, 1H), 7.88-7.80 (m, 1H), 7.80-7.73 (m, 1H), 7.48 (d, J = 7.6 Hz, |
| | 1H), 7.35-7.25 (m, 2H), 7.06-6.92 (m, 3H), 4.48-4.35 (m, 1H), 3.15-3.03 |
| | (m, 1H), 2.77-2.64 (m, 1H), 2.22 (s, 3H), 2.04-1.87 (m, 3H), 1.81-1.71 (m, |
| | 1H), 1.67-1.55 (m, 1H), 1.52-1.42 (m, 1H). |
| I-54 | MS m/z 404.2 [M + H]⁺; ¹H NMR (METHANOL-d4) δ: 8.95 (s, 1H), 8.92 (d, |
| | J = 5.8 Hz, 1H), 8.24 (d, J = 5.8 Hz, 1H), 7.63 (d, J = 7.9 Hz, 1H), 7.34 (d, J = 8.0 |
| | Hz, 1H), 7.29 (s, 1H), 4.55-4.67 (m, 1H), 3.16-3.29 (m, 1H), 2.79 (br s, 1H), |
| | 2.42 (s, 4H), 2.10 (br s, 1H), 1.86-1.98 (m, 1H), 1.79 (br dd, J = 10.1, 3.6 Hz, |
| | 1H), 1.70 (br s, 1H), 1.11-1.21 (m, 1H), NH and OH not observed. |
| I-64 | MS m/z 431.2 [M + H]⁺; ¹H NMR (METHANOL-d4) δ: 8.35-8.42 (m, 1H), 8.20 |
| | (s, 1H), 7.92-8.04 (m, 1H), 7.83-7.91 (m, 1H), 7.61-7.75 (m, 1H), 7.56 (br d, |
| | J = 7.0 Hz, 1H), 7.31-7.38 (m, 1H), 4.30-4.41 (m, 2H), 3.20 (br dd, J = 12.5, 9.4 |
| | Hz, 1H), 3.10 (br d, J = 10.4 Hz, 1H), 2.90 (br d, J = 14.6 Hz, 1H), 2.27 (br dd, |
| | J = 12.4, 3.9 Hz, 1H), 2.19 (s, 3H), 1.83-1.97 (m, 2H), 1.66 (br d, J = 12.5 Hz, |
| | 1H). NH and OH not observed. |
| I-66 | MS m/z 404.2 [M + H]⁺. ¹H NMR (400 MHz, CD₃OD) δ 9.15 (d, J = 4.5 Hz, 1H), |
| | 8.82 (d, J = 8.3 Hz, 1H), 8.54 (s, 1H, formic acid), 8.43 (d, J = 8.1 Hz, 1H), |
| | 7.96-7.86 (m, 1H), 7.28-7.20 (m, 2H), 4.60 (s, 1H), 2.94-2.77 (m, 1H), |
| | 2.61-2.38 (m, 5H), 2.18-2.03 (m, 1H), 2.02-1.89 (m, 1H), 1.90-1.59 (m, |
| | 2H). |
| I-67 | MS m/z 370.1 [M + H]⁺; ¹H NMR (500 MHz, CD₃OD) δ 8.90 (d, J = 4.5 Hz, |
| | 1H), 8.26 (s, 1H, formic acid), 7.84 (d, J = 8.3 Hz, 1H), 7.63 (dd, J = 8.4, 4.4 |
| | Hz, 1H), 7.16 (d, J = 8.0 Hz, 1H), 6.84 (d, J = 8.2 Hz, 1H), 6.82 (s, 1H), 4.43- |
| | 4.33 (m, 1H), 3.58-3.31 (m, 1H), 3.07-2.97 (m, 1H), 2.90-2.64 (m, 2H), 2.58 |
| | (s, 3H), 2.05-1.85 (m, 2H), 1.79-1.53 (m, 2H), NH and OH not observed |
| I-73 | MS m/z 366.1 [M + H]⁺; ¹H NMR (METHANOL-d₄) δ: 9.66 (s, 1H), 8.80 (d, |
| | J = 5.5 Hz, 1H), 7.46 (dd, J = 5.8, 0.8 Hz, 1H), 7.12 (dd, J = 6.8, 2.8 Hz, 1H), |
| | 6.94-7.00 (m, 2H), 4.57-4.65 (m, 1H), 3.94 (s, 3H), 3.03-3.24 (m, 1H), 2.63-2.79 (m, |
| | 1H), 2.29-2.41 (m, 5H), 2.01-2.13 (m, 1H), 1.83-1.93 (m, 1H), 1.59-1.82 (m, |
| | 2H). NH and OH not observed |
| I-85 | MS m/z 392.9 [M + H]⁺; ¹H NMR (DMSO-d6) δ: 8.48 (d, J = 8.3 Hz, 1H), 8.40 (s, |
| | 1H, formic acid), 7.57 (br d, J = 7.6 Hz, 1H), 7.45 (s, 1H), 7.34 (br d, J = 8.5 Hz, |
| | 1H), 7.28 (s, 1H), 4.26-4.37 (m, 1H), 3.01 (br d, J = 8.8 Hz, 1H), 2.67 (br d, |
| | J = 10.5 Hz, 1H), 2.21 (s, 3H), 1.86-2.03 (m, 3H), 1.70-1.81 (m, 1H), 1.60 (br d, |
| | J = 12.5 Hz, 1H), 1.30-1.45 (m, 1H), NH and OH not observed. |
| I-95 | MS m/z 417.2 [M + H]⁺; ¹H NMR (400 MHz, CD₃OD) δ 8.26 (d, J = 8.2 Hz, |
| | 1H), 7.94-7.88 (m, 1H), 7.86-7.80 (m, 1H), 7.63 (d, J = 8.2 Hz, 1H), 7.54 (d, |
| | J = 7.9 Hz, 1H), 7.30 (d, J = 7.9 Hz, 1H), 7.25 (d, J = 1.7 Hz, 1H), 4.59 (s, 1H), |
| | 3.29-3.20 (m, 1H), 3.16-2.98 (m, 2H), 2.95-2.82 (m, 1H), 2.64 (s, 3H), |
| | 2.27-2.14 (m, 1H), 2.09-1.61 (m, 5H). NH and OH not observed |
| I-108 | MS m/z 419.1 [M + H]⁺; ¹H NMR (DMSO-d6) δ: 10.36 (br s, 1H), 8.30 (d, J = 8.3 |
| | Hz, 1H), 7.87 (t, J = 7.2 Hz, 1H), 7.79 (t, J = 7.5 Hz, 1H), 7.52 (br d, J = 7.8 Hz, |
| | 2H), 7.45 (d, J = 8.0 Hz, 1H), 7.30 (d, J = 7.8 Hz, 1H), 7.28 (s, 1H), 3.74 (q, J = 6.4 |
| | Hz, 2H), 3.57-3.65 (m, 4H), 2.57-2.79 (m, 2H), 2H under DMSO peak. |
| I-109 | MS m/z 361.1 [M + H]⁺; ¹H NMR (DMSO-d6) δ: 7.95-8.11 (m, 1H), 7.65 (dt, |
| | J = 5.0, 2.4 Hz, 2H), 7.46 (br d, J = 7.8 Hz, 1H), 7.19-7.29 (m, 2H), 7.08-7.19 (m, |
| | 1H), 4.19 (br d, J = 6.8 Hz, 2H), 3.99 (s, 1H), 3.22-3.45 (m, 4H), 2.71-2.83 (m, |
| | 1H). |
| I-113 | MS m/z 418.3 [M + H]⁺; ¹H NMR (400 MHz, CD₃OD) δ 9.66 (s, 1H), 8.94 (d, J = |
| | 5.6 Hz, 1H), 8.55 (s, 1H, formic acid), 7.56 (dd, J = 15.4, 6.7 Hz, 2H), 7.34 |
| | (d, J = 8.0 Hz, 1H), 7.28 (s, 1H), 4.17-4.03 (m, 1H), 4.03-3.92 (m, 1H), |
| | 3.87-3.70 (m, 1H), 3.69-3.49 (m, 2H), 3.21-3.01 (m, 2H), 2.38-2.21 (m, 1H), |
| | 2.18-1.88 (m, 3H), 1.43 (t, J = 7.3 Hz, 3H). NH and OH not observed |
| I-117 | MS m/z 423.8 [M + H]⁺; ¹H NMR (400 MHz, METHANOL-d4) δ: 7.83 (br d, |
| | J = 7.6 Hz, 1H), 7.10-7.34 (m, 2H), 4.37 (br s, 4H), 3.42-3.55 (m, 1H), 3.09-3.31 |
| | (m, 1H), 2.78-2.93 (m, 1H), 2.74 (s, 3H), 2.41-2.64 (br s, 3H), 1.96-2.15 (m, |
| | 3H), 1.77-1.94 (m, 2H), 1.65-1.77 (m, 1H). NH and OH not observed |
| I-119 | MS m/z 418.2 [M + H]⁺; ¹H NMR (METHANOL-d₄) δ: 9.72 (s, 1H), 8.87 (d, |
| | J = 5.8 Hz, 1H), 8.26 (s, 3H, formic acid), 7.61 (br d, J = 7.8 Hz, 1H), 7.43-7.53 |
| | (m, 2H), 7.38 (d, J = 5.8 Hz, 1H), 4.66-4.80 (m, 1H), 3.93-3.83 (m, 1H), 3.80 (s, |
| | 3H), 3.38-3.55 (m, 1H), 3.05-3.17 (m, 2H), 2.92 (s, 3H), 2.15-2.37 (m, 2H), |
| | 1.86-2.07 (m, 2H), NH and OH not observed. |
| I-120 | MS m/z 452.9 [M + H]⁺; ¹H NMR (400 MHz, METHANOL-d4) δ: 8.45-8.37 |
| | (m, 2 H, including formic acid), 7.96 (t, J = 7.69 Hz, 1 H), 7.87 (t, J = 7.63 Hz, |
| | 1 H), 7.82-7.72 (m, 2 H), 7.70 (s, 1 H), 7.52 (d, J = 8.13 Hz, 1 H), 6.90 (td, J = |
| | 73.92 Hz, 11.51 Hz, 1 H), 4.77-4.60 (m, 1 H), 3.96-3.75 (m, 1 H), 3.54-3.37 |
| | (m, 1 H), 3.16-2.98 (m, 2 H), 2.91 (s, 3 H), 2.36-2.12 (m, 2 H), 2.06-1.82 |
| | (m, 2 H). 1H not observed (NH). |
| I-121- | MS m/z 454.2 [M + H]⁺; ¹H NMR (400 MHz, DMSO-d6) δ: 9.83 (s, 1 H), 8.86 |
| | (d, J = 5.63 Hz, 1 H), 7.85-7.76 (m, 3 H), 7.75 (s, 1 H), 7.35 (t, J = 73.17 Hz, |
| | 1 H), 7.27 (d, J = 5.63 Hz, 1 H), 4.53-4.44 (m, 1 H), 3.15-3.06 (m, 1 H), |
| | 2.77-2.68 (m, 1 H), 2.23 (s, 3 H), 2.05-1.85(m, 3 H), 1.83-1.73 (m, 1 H), |
| | 1.70-1.54 (m, 1 H), 1.52-1.41 (m, 1 H) |
| I-127 | MS m/z 405.3 [M + H]⁺; ¹H NMR (400 MHz, DMSO-d6) δ 8.48 (d, J = 8.0 Hz, |
| | 1H), 7.95-7.88 (m, 2H), 7.88-7.77 (m, 3H), 7.52-7.46 (m, 1H), 7.35 (d, J = |
| | 7.2 Hz, 1H), 4.54-4.41 (m, 1H), 3.19-3.06 (m, 1H), 2.84-2.70 (m, 1H), |
| | 2.28 (s, 3H), 2.09-1.91 (m, 3H), 1.84-1.74 (m, 1H), 1.71-1.59 (m, 1H), |
| | 1.52-1.42 (m, 1H). |
| I-128 | MS m/z 406.2 [M + H]⁺; ¹H NMR (MeOH-d4) δ: 9.77 (s, 1H), 8.95 (d, J = 5.5 |
| | Hz, 1H), 8.47 (s, 1H, formic acid), 7.50-7.90 (m, 3H), 7.51 (br s, 1H), 4.72-4.79 |
| | (m, 1H), 3.60-3.75 (m, 1H), 3.29-3.32 (m, 1H), 2.80-3.00 (m, 2H), 2.67 (s, 3H), |
| | 2.10-2.30 (m, 2H), 1.83-2.00 (m, 2H). —NH not observed |
| I-129 | MS m/z 372.1, 374.1 [M + H]⁺; ¹H NMR (MeOH-d4) δ: 9.76 (s, 1H), 8.94 (d, J = |
| | 5.5 Hz, 1H), 8.47 (s, 1H, formic acid), 7.62 (t, J = 8 Hz, 1H), 7.45-7.55 (m, |
| | 3H), 4.70-4.78 (m, 1H), 3.65-3.75 (m, 1H), 2.85-3.00 (m, 2H), 2.82 (s, 3H), |
| | 2.10-2.28 (m, 2H), 1.78-2.02 (m, 2H). One proton is hidden under the residual |
| | deutero methanol peak at 3.33 ppm. |
| I-131 | MS m/z 412.0 [M + H]⁺; ¹H NMR (CD₃OD) δ: 8.31-8.47 (m, 1H), 8.19 (br d, |
| | J = 7.6 Hz, 1H), 7.81-8.01 (m, 4H), 7.60 (br d, J = 8.1 Hz, 1H), 4.65 (dt, J = 8.3, |
| | 4.0 Hz, 1H), 2.74-2.97 (m, 2H), 2.50 (s, 5H), 2.08-2.16 (m, 1H), 1.91-2.01 (m, |
| | 1H), 1.71-1.89 (m, 2H). |
| I-138 | MS m/z 438.1 [M + H]⁺; ¹H NMR (CHLOROFORM-d) δ: 9.50 (br s, 1H), 8.89 |
| | (d, J = 5.8 Hz, 1H), 8.15 (s, 1H), 7.87 (d, J = 8.0 Hz, 1H), 7.56 (d, J = 8.0 Hz, 1H), |
| | 7.29 (d, J = 5.5 Hz, 1H), 7.01 (t, J = 55.5 Hz, 1H), 6.69 (br dd, J = 7.2, 4.1 Hz, 1H), |
| | 4.81 (br s, 1H), 2.84 (br s, 2H), 2.44-2.61 (m, 1H), 2.36 (s, 3H), 2.06-2.28 (m, |
| | 2H), 1.80-1.98 (m, 1H), 1.67-1.73 (m, 2H) |
| I-139 | MS m/z 370.0 [M + H]⁺; ¹H NMR (DMSO-d6) δ: 9.98 (br s, 1H), 9.81 (s, 1H), |
| | 8.84 (d, J = 5.5 Hz, 1H), 7.66 (br d, J = 7.3 Hz, 1H), 7.36 (t, J = 8.2 Hz, 1H), |
| | 7.04-7.12 (m, 2H), 6.97-7.02 (m, 1H), 4.42-4.53 (m, 1H), 3.09-3.17 (m, 1H), |
| | 2.71-2.80 (m, 1H), 2.19-2.31 (m, 3H), 1.89-2.10 (m, 3H), 1.79 (br d, J = 11.0 Hz, 1H), |
| | 1.63 (br d, J = 12.5 Hz, 1H), 1.49 (br dd, J = 10.7, 4.9 Hz, 1H) |
| I-140 | MS m/z 361.0 [M + H]⁺; ¹H NMR (400 MHz, DMSO-d6) δ 10.4 (bs, 1H), 9.82 |
| | (s, 1H), 8.85 (d, J = 4.0 Hz, 1H), 7.78-7.75 (m, 1H), 7.56-7.52 (m, 1H), 7.46 (d, |
| | J = 4.0 Hz, 1H), 7.32 (d, J = 8.0 Hz, 1H), 7.17 (d, J = 8.0 Hz, 1H), 4.49 (bs, 1H), |
| | 3.35-3.30 (m, 1H), 3.12-3.08 (m, 1H), 2.23 (d, J = 8.0 Hz, 3H), 2.03-1.94 (m, |
| | 3H), 1.78-1.76 (m, 1H), 1.64-1.61 (m, 1H), 1.50-1.44 (m, 1H). |
| I-154 | MS m/z 422.0 [M + H]⁺; ¹H NMR (400 MHz, METHANOL-d4) δ: 7.61 (br d, |
| | J = 8.0 Hz, 1H), 7.23-7.29 (m, 1H), 7.18-7.22 (m, 1H), 4.38 (br s, 1H), 3.52-3.74 |
| | (m, 1H), 3.35-3.51 (m, 2H), 3.01 (br s, 1H), 2.77-2.96 (m, 4H), 2.50-2.62 (m, |
| | 5H), 2.12 (br s, 2H), 1.88-2.09 (m, 4H), 1.79 (br s, 1H). |
| I-155 | MS m/z 388.4, 390.3 [M + H]⁺; ¹H NMR (METHANOL-d4) δ: 7.55 (br d, J = 8.5 |
| | Hz, 1H), 6.90-6.99 (m, 2H), 4.36 (br dd, J = 3.1, 2.1 Hz, 1H), 3.50 (br s, 1H), |
| | 3.12-3.25 (m, 4H), 2.96-3.08 (m, 1H), 2.62-2.73 (m, 1H), 2.46-2.58 (m, 4H), |
| | 2.38 (s, 3H), 1.93-2.08 (m, 3H), 1.80-1.89 (m, 1H), 1.67-1.80 (m, 1H), |
| | 1.54-1.66 (m, 1H), OH and NH not observed. |
| I-159 | MS m/z 350.0 [M + H]⁺; ¹H NMR (400 MHz, DMSO-d6) δ 11.8 (bs, 1H), 8.34 |
| | (d, J = 8 Hz, 1H), 7.83-7.80 (m, 2H), 7.76-7.72 (m, 2H),7.50 (d, J = 8 Hz, 1H), |
| | 7.05 (bs, 1H), 4.4 (bs, 1H), 3.34-3.30 (m, 2H), 2.84-2.82 (m, 2H), 2.35-2.32 |
| | (bs, 3H), 2.01-1.99 (m, 1H), 1.95 (s, 3H), 1.80-1.78 (m, 1H), 1.65-1.62 (m, 1H), |
| | 1.50-1.45 (m, 1H). |
| I-160 | MS m/z 364.1 [M + H]⁺; ¹H NMR (DMSO-d6) δ: 8.39 (d, J = 8.0 Hz, 1H), 7.84 (t, |
| | J = 7.1 Hz, 1H), 7.73-7.80 (m, 1H), 7.66 (d, J = 7.3 Hz, 1H), 7.35 (d, J = 7.8 Hz, |
| | 1H), 7.11 (br d, J = 7.5 Hz, 1H), 7.03 (d, J = 7.3 Hz, 1H), 4.36-4.46 (m, 1H), 3.74 |
| | (s, 3H), 3.05-3.13 (m, 1H), 2.68-2.76 (m, 1H), 2.51 (s, 3H), 2.22 (s, 3H), |
| | 1.84-2.05 (m, 3H), 1.70-1.81 (m, 1H), 1.61 (br d, J = 11.8 Hz, 1H), 1.46 (br dd, |
| | J = 11.5, 3.3 Hz, 1H) |
| I-165 | MS m/z 366.1 [M + H]⁺ 369.7; ¹H NMR (400 MHz, METHANOL-d4) δ: 8.43 |
| | (br s, 1H, formic acid peak), 8.25 (d, J = 8.3 Hz, 1H), 7.84 (s, 1H), 7.77 (s, 1H), |
| | 7.45-7.62 (m, 1H), 7.13 (br d, J = 1.5 Hz, 2H), 4.40-4.69 (m, 1H), 3.26-3.56 (m, |
| | 1H), 2.89-3.07 (m, 1H), 2.53 (br s, 5H), 2.34 (s, 3H), 1.97-2.13 (m, 1H), |
| | 1.86-1.97 (m, 1H), 1.62-1.86 (m, 2H). OH and NH not observed. |
| I-166 | MS m/z 388.2 [M + H]⁺; ¹H NMR (CD₃OD) δ: 9.73 (s, 1H), 8.90 (d, J = 5.8 Hz, |
| | 1H), 7.84-7.92 (m, 4H), 7.72 (d, J = 5.6 Hz, 1H), 4.58-4.71 (m, 1H), 3.06-3.25 |
| | (m, 1H), 2.73 (br s, 1H), 2.30-2.45 (m, 4H), 2.08 (br s, 1H), 1.85-1.95 (m, 1H), |
| | 1.66-1.85 (m, 2H) |
| I-167 | MS m/z 411.2 [M + H]⁺; ¹H NMR (400 MHz, DMSO-d6) δ9.74 (s, 1H), 8.93 (d, |
| | J = 5.6 Hz, 1H), 7.86-7.83 (m, 2H), 7.77-7.75(m, 1H), 7.44-7.42 (m, 1H), 6.92(t, |
| | J = 72.8 Hz, 1H), 4.75-4.68 (m, 1H), 4.03-4.01 (m, 1H), 3.61-3.57(m, 1H), |
| | 3.06-3.00 (m, 1H), 2.97 (s, 3H), 2.94-2.87 (m, 1H), 2.37-2.32 (m, 1H), 2.21-2.18(m, |
| | 1H), 2.08-1.84 (m, 2H). NH not observed |
| I-168 | MS m/z 400.2 [M + H]⁺; ¹H NMR (400 MHz, CD₃OD) δ 9.87(s, 1H), 9.05(d, J = |
| | 5.64 Hz, 1H), 7.63(dd, J = 7.56, 1.93 Hz, 1H), 7.55(d, J = 5.63 Hz, 1H), |
| | 7.53-7.40 (m, 2H), 6.46 (t, J = 73.6 Hz, 1H), 4.70 (d, J = 12.9 Hz, 1H), 3.98 (d, J = |
| | 11.8 Hz, 1H), 3.60 (d, J = 12.5 Hz, 1H), 3.03 (d, J = 11.3 Hz, 2H), 2.98 (s, 3H), |
| | 2.45 (s, 3H), 2.35 (s, 1H), 2.21-2.12 (m, 1H), 2.10-1.8 (m, 2H).—NH not |
| | observed |
| I-169 | MS m/z 481.0 [M + H]⁺; ¹H NMR (400 MHz, DMSO-d6) δ 9.82 (s, 1H), 8.82 (d, |
| | J = 5.6 Hz, 1H), 8.33 (s, 1H), 8.20 (d, J = 8.0 Hz, 1H), 7.82 (d, J = 7.6 Hz, 1H), |
| | 7.76 (d, J = 8.0 Hz, 1H), 7.31-7.30 (m, 1H), 7.12 (d, J = 5.6 Hz, 1H), 4.51-4.50 |
| | (m, 1H), 3.29 (s, 1H), 3.18 (s, 1H), 2.82 (s, 1H), 2.43 (d, J = 4.8 Hz, 3H), |
| | 2.33-2.31 (m, 3H), 2.06-2.03 (m, 2H), 1.82-1.80 (m, 1H), 1.66-1.62 (m, 1H), |
| | 1.53-1.50 (m, 1H). |
| I-171 | MS m/z 415.4 [M—H₂O + H]⁺; ¹H NMR (400 MHz, DMSO-d6) δ 9.83 (s, 1H), |
| | 8.93 (d, J = 5.6 Hz, 1H), 8.18 (s, 1H), 7.45 (d, J = 8.4 Hz, 1H), 7.32 (d, J = 5.6 |
| | Hz, 1H), 7.29 (t, J = 36 Hz, 1H), 7.03 (t, J = 2.0, 8.4 Hz 1H), 4.71-4.65 (m, 1H), |
| | 3.89(s, 3H), 3.55-3.48 (m, 1H), 2.99-2.83 (m, 5H), 2.18 (d, J = 10.4 Hz, 1H), |
| | 2.06-2.00 (m, 1H), 1.92-1.69 (m, 2H) |
| I-175 | MS m/z [M + H]⁺: 366.4; ¹H NMR (400 MHz, DMSO-d6) δ 9.84 (s, 1 H), 8.84 |
| | (d, J = 5.63 Hz, 1 H), 7.51 (d, J = 7.50 Hz, 1 H), 7.34 (d, J = 5.63 Hz, 1 H), 7.25 |
| | (d, J = 8.25 Hz, 1 H), 6.63-6.50 (m, 2 H), 4.48-4.37 (m, 1 H), 3.79 (s, 3 H), |
| | 3.10 (d, J = 8.25 Hz, 1 H), 2.72 (d, J = 10.8 Hz, 1 H), 2.22 (s, 3 H), 2.06-1.85 |
| | (m, 3 H), 1.83-1.70 (m, 1 H), 1.69-1.54 (m, 1 H), 1.53-1.37 (m, 1 H). 1H |
| | not observed (OH) |
| I-177 | MS m/z [M + H]⁺: 456.4; ¹H NMR (400 MHz, DMSO-d6) δ 9.84 (s, 1 H), 8.84 |
| | (d, J = 5.63 Hz, 1 H), 8.32-8.27 (m, 1 H), 8.25 (d, J = 8.63 Hz, 1 H), |
| | 7.90-7.84 (m, 1 H), 7.79 (d, J = 7.63 Hz, 1 H), 7.14 (d, J = 5.63 Hz, 1 H), 4.54-4.43 |
| | (m, 1 H), 3.16-3.02 (m, 1 H), 2.80-2.68 (m, 1 H), 2.23 (s, 3 H), 2.07-1.85 |
| | (m, 3 H), 1.82-1.73 (m, 1 H), 1.68-1.56 (m, 1 H), 1.55-1.40 (m, 1 H) |
| I-178 | MS m/z 418.0 [M + H]⁺; ¹H NMR (400 MHz, CD₃OD) δ 9.71(s, 1H), 8.85(d, J = |
| | 6.0 Hz, 1H), 7.35(d, J = 6.4 Hz, 1H), 7.30-7.25 (m, 2H), 6.65 (t, J = 73.6 Hz, 1H), |
| | 4.66-4.59 (m, 1H), 3.13-3.12 (m, 1H), 2.69 (s, 1H), 2.40 (d, J = 1.65 Hz, 3H), |
| | 2.35 (s, 3H), 2.34-2.15 (m, 2H), 2.07 (s, 1H), 1.89 (dd, J = 9.3, 4.1 Hz, 1H), |
| | 1.83-1.50 (m, 2H). |
| I-183 | MS m/z 454.1 [M + H]⁺; ¹H NMR (400 MHz, CD₃OD) δ 9.12 (dd, J = 4.4, 1.6 |
| | Hz, 1H), 7.91 (dd, J = 8.4, 1.6 Hz, 1H), 7.82 (dd, J = 8.4, 4.3 Hz, 1H), |
| | 7.80-7.75 (m, 2H), 7.68 (s, 1H), 6.88 (t, J = 72.9 Hz, 1H), 4.59-4.48 (m, 1H), 3.12 |
| | (s, 1H), 2.84-2.62 (m, 1H), 2.59-2.32 (m, 5H), 2.15-1.99 (m, 1H), |
| | 1.98-1.85 (m, 1H), 1.83-1.62 (m, 2H). |
| I-184 | MS m/z [M + H]⁺: 418.4; ¹H NMR (400 MHz, DMSO-d6) δ 9.81 (s, 1 H), 8.83 |
| | (d, J = 5.50 Hz, 1 H), 7.66 (d, J = 7.50 Hz, 1 H), 7.52-7.35 (m, 3 H), 7.07 (d, |
| | J = 5.50 Hz, 1 H), 4.51-4.39 (m, 1 H), 3.93 (s, 3 H), 3.16-2.98 (m, 1 H), |
| | 2.80-2.68 (m, 1 H), 2.22 (s, 3 H), 2.07-1.84 (m, 3 H), 1.81-1.71 (m, 1 H), |
| | 1.69-1.54 (m, 1 H), 1.52-1.38 (m, 1 H) |
| I-184 | MS m/z 454.2 [M + H]⁺; ¹H NMR (400 MHz, CD₃OD) δ 9.08 (dd, J = 4.4, 1.6 |
| | Hz, 1H), 8.78 (dd, J = 8.5, 1.6 Hz, 1H), 8.48 (s, 1H, formic acid), 7.89 (dd, J = |
| | 8.5, 4.4 Hz, 1H), 7.80-7.70 (m, 2H), 7.59 (s, 1H), 6.85 (t, J = 73.5 Hz, 1H), |
| | 4.72-4.60 (m, 1H), 3.74-3.53 (m, 1H), 3.26-3.15 (m, 1H), 2.95-2.64 (m, |
| | 5H), 2.28-2.15 (m, 1H), 2.15-2.03 (m, 1H), 2.01-1.74 (m, 2H), NH not |
| | observed. |
| I-185 | MS m/z 418.2 [M + H]⁺; ¹H NMR (400 MHz, CD₃OD) δ 8.97 (s, 1H), 8.95 (d, J = |
| | 5.8 Hz, 1H), 8.54 (s, 1H, formic acid), 8.18 (d, J = 5.8 Hz, 1H), 7.63 (d, J = |
| | 7.9 Hz, 1H), 7.36 (d, J = 8.2 Hz, 1H), 7.29 (s, 1H), 4.66-4.55 (m, 1H), 3.53- |
| | 3.38 (m, 1H), 3.29-3.20 (m, 1H), 3.20-3.03 (m, 2H), 2.78 (s, 3H), 2.35-2.19 |
| | (m, 1H), 2.15-1.87 (m, 4H), 1.83-1.70 (m, 1H), NH and OH not |
| | observed |
| I-186 | MS m/z 418.2 [M + H]⁺; 1H NMR (400 MHz, CD₃OD) δ 9.70 (s, 1H), 8.87 (d, J = |
| | 5.6 Hz, 1H), 8.40 (s, 1H, formic acid), 7.57 (d, J = 7.9 Hz, 1H), 7.48 (d, J = |
| | 5.6 Hz, 1H), 7.33 (d, J = 7.9 Hz, 1H), 7.28 (s, 1H), 4.70-4.58 (m, 1H), |
| | 3.82-3.69 (m, 1H), 3.65-3.54 (m, 1H), 3.53-3.43 (m, 1H), 3.42-3.33 (m, 1H), |
| | 3.03 (s, 3H), 2.40-2.27 (m, 1H), 2.25-2.13 (m, 1H), 2.12-1.95 (m, 3H), |
| | 1.89-1.72 (m, 1H), NH and OH not observed. |
| I-187 | MS m/z 418.2 [M + H]⁺; ¹H NMR (400 MHz, CD₃OD) δ 9.70 (s, 1H), 8.86 (d, J = |
| | 5.6 Hz, 1H), 8.43 (s, 1H, formic acid), 7.57 (d, J = 7.9 Hz, 1H), 7.48 (d, J = |
| | 5.4 Hz, 1H), 7.37-7.30 (m, 1H), 7.28 (d, J = 1.7 Hz, 1H), 4.71-4.58 (m, 1H), |
| | 3.79-3.67 (m, 1H), 3.64-3.54 (m, 1H), 3.52-3.42 (m, 1H), 3.40-3.33 (m, |
| | 1H), 3.02 (s, 3H), 2.38-2.27 (m, 1H), 2.24-2.13 (m, 1H), 2.11-1.96 (m, |
| | 3H), 1.89-1.71 (m, 1H), NH and OH not observed |
| I-191 | MS m/z 470.0 [M + H]⁺; ¹H NMR (400 MHz, DMSO-d6) δ 9.82 (s, 1H), 8.87 |
| | (d, J = 5.6 Hz, 1H), 7.76 (d, J = 7.2 Hz, 1H), 7.70 (d, J = 8.4 Hz, 1H), 7.47 (s, |
| | 2H), 7.29 (t, J = 72.4 Hz, 1H), 7.26 (d, J = 5.6 Hz, 1H), 4.49-4.41 (m, 1H), |
| | 3.12-3.08 (m, 1H), 2.75-2.73 (m, 1H), 2.23 (s, 3H), 2.03-1.89 (m, 3H), |
| | 1.79-1.75 (m, 1H ), 1.63-1.60 (m, 1H), 1.51-1.46 (m, 1H). |
| I-192 | MS m/z 466.3 [M + H]⁺; ¹H NMR (400 MHz, DMSO-d6) δ 9.85 (s, 1H), 8.84 |
| | (d, J = 5.6 Hz, 1H ), 8.40 (s, 1H), 8.30 (d, J = 8.0 Hz, 1H), 7.90 (d, J = 7.6 Hz, |
| | 1H), 7.85-7.83 (m, 1H), 7.18 (d, J = 6.0 Hz, 1H), 4.52-4.48 (m, 1H), 3.39 (s, |
| | 3H), 3.30-3.27 (m, 1H), 3.20-3.17 (m, 1H), 2.83-2.78 (m, 1H), 2.35-2.29 (m, |
| | 3H), 2.08-2.00 (m, 2H), 1.83-1.80 (m, 1H), 1.67-1.63 (m, 1H), 1.55-1.53 (m, |
| | 1H). |
| I-198 | MS m/z 386.0, 388.0 [M + H]⁺; ¹H NMR (400 MHz, CD₃OD) δ 9.75 (s, 1H), |
| | 8.86 (d, J = 5.6 Hz, 1H), 7.34 (s, 1H), 7.32-7.20 (m, 2H), 4.64 (s, 1H), 3.13 (s, |
| | 1H), 2.72 (s, 1H), 2.36 (s, 3H), 2.35-2.19 (m, 2H), 2.10 (s, 3H), 1.96-1.83 (m, |
| | 1H), 1.88 (s, 1H), 1.84-1.58 (m, 2H). |
| I-221 | MS m/z 390.2, 392.2 [M + H]⁺; ¹H NMR (400 MHz, CD₃OD) δ 9.97 (s, 1H), |
| | 8.95 (d, J = 5.6 Hz, 1H), 7.47 (d, J = 5.2 Hz, 1H), 7.40 (q, J = 10.4 Hz, 2.8 |
| | Hz, 2H), 4.85-4.74 (m, 1H), 4.02 (d, J = 12 Hz, 1H), 3.59 (d, J = 12.4 Hz, 1H), |
| | 3.07-2.87 (m, 2H), 2.96 (s, 3H), 2.33 (d, J = 11.6 Hz, 1H), 2.19 (d, J = 14.8 Hz, |
| | 1H), 2.00-1.84(m, 2H). |
| I-222 | MS m/z 420.5 [M + H]⁺; ¹H NMR (400 MHz, DMSO-d6) δ 9.77(s, 1H), 8.87(d, |
| | J = 5.6 Hz, 1H), 7.62(s, 1H), 7.51(d, J = 8.8 Hz, 1H), 7.26 (d, J = 5.6 Hz, 1H), |
| | 4.65(s, 1H), 3.13-3.12(m, 1H), 2.71 (s, 1H), 2.36(s, 3H), 2.32-2.23 (m, 2H), |
| | 2.19(s, 3H), 2.10-2.09(m, 1H), 1.91-1.88(m, 1H), 1.82-1.63 (m, 2H), NH not |
| | observed |
| I-223 | MS m/z 382.1 [M + H]⁺. ¹H NMR (400 MHz, CD₃OD) δ 9.86 (s, 1H), 9.05 (d, J = |
| | 5.6 Hz, 1H), 7.94 (dd, J = 8.4, 2.2 Hz, 1H), 7.88 (d, J = 2.2 Hz, 1H), |
| | 7.71-7.59 (m, 2H), 4.69 (d, J = 12.4 Hz, 1H), 3.99 (d, J = 11.6 Hz, 1H), 3.61 (d, J = |
| | 12.4 Hz, 1H), 3.06 (d, J = 15.4 Hz, 1H), 2.98 (s, 3H), 2.35 (s, 1H), 2.20 (d, J = |
| | 14.8 Hz, 1H), 2.10-1.94 (m, 2H), 1.87 (d, J = 13.2 Hz, 1H), NH and OH not |
| | observed. |
| I-229 | MS m/z 427.5 [M + H]⁺; ¹H NMR (400 MHz, CD₃OD) δ 8.97 (s, 1H), 8.94 (d, J = |
| | 5.8 Hz, 1H), 8.49 (s, 1H, formic acid), 8.23 (dd, J = 5.8, 1.0 Hz, 1H), 7.63 (d, |
| | J = 7.9 Hz, 1H), 7.36 (d, J = 7.9 Hz, 1H), 7.29 (d, J = 1.7 Hz, 1H), 7.05 (d, J = |
| | 16.3 Hz, 2H), 5.03-4.91 (m, 1H), 4.70-4.61 (m, 1H), 4.17-4.03 (m, 1H), |
| | 3.20-3.09 (m, 1H), 3.09-2.96 (m, 1H), 2.53-2.40 (m, 1H), 2.39-2.26 (m, |
| | 1H), NH and OH not observed. |
| I-231 | MS m/z 440.5 [M + H]⁺; ¹H NMR (400 MHz, DMSO-d6) δ 8.99 (d, J = 5.8 Hz, |
| | 1H), 8.82 (s, 1H), 8.36 (d, J = 5.9 Hz, 1H), 8.31 (s, 1H), 7.84 (s, 2H), 7.74 (s, |
| | 1H), 7.61 (d, J = 8.25 Hz, 1H), 7.38 (t, J = 72.92 Hz, 1H), 4.45-4.31 (m, 1H), |
| | 3.33-3.25 (m, 1H), 2.95 (d, J = 12.1 Hz, 1H), 2.70-2.54 (m, 2H), 2.15-2.03 |
| | (m, 1H), 1.83-1.73 (m, 1H), 1.72-1.48 (m, 2H) |
| I-234 | MS m/z 398.2 [M − H]−; ¹H NMR (400 MHz, METHANOL-d4) δ = 9.72 (s, 1H), |
| | 8.84 (d, J = 5.6 Hz, 1H), 7.54 (d, J = 7.6 Hz, 1H), 7.42-7.31 (m, 3H), |
| | 7.13-6.70 (m, 1H), 4.71-4.61 (m, 1H), 3.79 (s, 3H), 2.88 (br s, 1H), 2.60-2.39 (m, |
| | 6H), 2.12 (br s, 1H), 2.02-1.91 (m, 1H), 1.91-1.65 (m, 2H); NH wasn′t |
| | observed |
| I-236 | MS m/z 443.4 [M + H]⁺; ¹H NMR (400 MHz, DMSO-d6) δ 8.18 (s, 1H, formic |
| | acid), 7.77-7.67 (m, 2H), 7.64 (s, 1H), 7.37 (t, J = 73.17 Hz, 1H), 6.19 (d, J = |
| | 8.50 Hz, 1H), 4.34-4.23 (m, 1H), 3.06-2.95 (m, 1H), 2.76 (t, J = 7.38 Hz, |
| | 2H), 2.73-2.68 (m, 3H), 2.23 (s, 3H), 2.06-1.84 (m, 5H), 1.79-1.68 (m, |
| | 1H), 1.65-1.50 (m, 1H), 1.46-1.31 (m, 1H) |
| I-239 | MS m/z 404 [M + H]⁺; ¹H NMR (400 MHz, CD₃OD) d: 9.74 (s, 1H), 8.86 (d, J = |
| | 5.6 Hz, 1H), 7.85 (d, J = 1.6 Hz, 1H), 7.73 (d, J = 8.0 Hz, 1H), 7.51 (d, J = |
| | 8.0 Hz, 1H), 7.34 (d, J = 5.6 Hz, 1H), 6.75 (t, J = 55.2 Hz, 1H), 4.67-4.62 (m, |
| | 1H), 3.22-3.21 (m, 1H), 2.80-2.79 (m, 1H), 2.43 (s, 3H), 2.42-2.31 (m, |
| | 2H), 2.12-2.09 (m, 1H), 1.95-1.91 (m, 1H), 1.81-1.71 (m, 2H), NH not |
| | observed. |
| I-242 | MS m/z 490.7 [M + H]+; ¹H NMR (400 MHz, METHANOL-d4) δ = 9.73 (s, |
| | 1H), 8.87 (d, J = 5.6 Hz, 1H), 7.41 (d, J = 5.6 Hz, 1H), 7.25 (d, J = 8.4 Hz, 1H), |
| | 6.93 (dd, J = 2.4, 8.4 Hz, 1H), 6.66 (s, 1H), 4.77-4.60 (m, 1H), 3.87 (s, 3H), |
| | 3.47-3.36 (m, 1H), 3.05-2.92 (m, 1H), 2.58 (s, 5H), 2.24-2.10 (m, 1H), |
| | 2.07-1.97 (m, 1H), 1.93-1.72 (m, 2H), 1.58-1.42 (m, 1H), 0.81-0.67 (m, 2H), |
| | 0.67-0.50 (m, 2H); NH wasn′t observed |
| I-245 | MS m/z 375.5 [M + H]⁺; ¹H NMR (400 MHz, METHANOL-d4) δ 8.53 (s, 1H, |
| | formic acid), 7.56-7.49 (m, 1H), 7.47-7.42 (m, 1H), 7.41-7.33 (m, 1H), |
| | 7.33-7.27 (m, 1H), 6.76 (t, J = 75.17 Hz, 1H), 4.51-4.42 (m, 1H), 3.57-3.41 |
| | (m, 1 H), 3.19-3.05 (m, 1 H), 2.88 (t, J = 7.3 Hz, 2H), 2.80 (t, J = 7.5 Hz, |
| | 2H), 2.76-2.60 (m, 5H), 2.14 (s, 4H), 1.92-1.76 (m, 1H), 1.76-1.60 (m, |
| | 1H). 1H not observed (NH) |
| I-249 | MS m/z 388.7 [M + H]⁺; ¹H NMR (400 MHz, METHANOL-d4) δ = 9.78 (s, |
| | 1H), 8.93 (d, J = 5.6 Hz, 1H), 7.75 (t, J = 7.4 Hz, 1H), 7.63 (d, J = 7.9 Hz, 1H), |
| | 7.60-7.55 (m, 1H), 7.55-7.44 (m, 1H), 7.13-6.77 (m, 1H), 4.80-4.66 (m, |
| | 1H), 3.51 (br d, J = 9.4 Hz, 1H), 3.20-3.02 (m, 1H), 2.78-2.75 (m, 2H), 2.67 |
| | (s, 3H), 2.21-2.14 (m, 1H), 2.13-2.03 (m, 1H), 1.95-1.77 (m, 2H); NH |
| | wasn′t observed |
| I-250 | MS m/z 409.4 [M + H]⁺; ¹H NMR (METHANOL-d4) δ: 8.56 (br s, 1H), 8.33 (d, |
| | J = 2.4 Hz, 1H), 7.87 (br d, J = 8.1 Hz, 1H), 7.12-7.31 (m, 2H), 4.34-4.60 (m, 1H), |
| | 3.59-3.88 (m, 1H), 3.28-3.44 (m, 1H), 2.92-3.07 (m, 1H), 2.82 (br s, 4H), 2.10 |
| | (m, 2H), 1.88 (m, 2H) |
| I-254 | MS m/z 364.8 [M + H]⁺; ¹H NMR (400 MHz, METHANOL-d4) δ = 9.73 (s, |
| | 1H), 8.96-8.77 (m, 1H), 7.39-7.16 (m, 2H), 7.03-6.86 (m, 2H), 4.76-4.53 |
| | (m, 1H), 3.88 (s, 3H), 3.27-3.13 (m, 1H), 2.86-2.71 (m, 1H), 2.42 (s, 5H), |
| | 2.09 (s, 4H), 2.00-1.87 (m, 1H), 1.87-1.63 (m, 2H); NH wasn′t observed |
| I-258 | MS m/z 406.9 [M + H]⁺; ¹H NMR (400 MHz, DMSO-d6) δ 7.39-7.25 (m, 3 H), |
| | 6.02 (d, J = 7.75 Hz, 1 H), 4.30-4.19 (m, 1 H), 3.88 (s, 3 H), 3.01-2.90 (m, 1 |
| | H), 2.75 (t, J = 7.38 Hz, 2 H), 2.69-2.62 (m, 1 H), 2.55 (t, J = 7.75 Hz, 2 H), |
| | 2.20 (s, 3 H), 2.03-1.83 (m, 5 H), 1.74-1.66 (m, 1 H), 1.62-1.49 (m, 1 H), |
| | 1.42-1.29 (m, 1 H) |
| I-259 | MS m/z 445.0 [M + H]⁺; 1H NMR (400 MHz, DMSO-d6) δ 8.18 (s, 1 H), 8.15 |
| | (d, J = 8.25 Hz, 1 H), 7.76 (d, J = 7.88 Hz, 1 H), 6.19 (d, J = 8.00 Hz, 1 H), |
| | 4.33-4.21 (m, 1 H), 2.96 (d, J = 9.01 Hz, 1 H), 2.77 (t, J = 7.44 Hz, 2 H), |
| | 2.70-2.64 (m, 1 H), 2.60 (t, J = 6.75 Hz, 2 H), 2.20 (s, 3 H), 2.01 (quin, J = 7.57 Hz, |
| | 2 H), 1.96-1.84 (m, 3 H), 1.70 (d, J = 3.63 Hz, 1 H), 1.63-1.49 (m, 1 H), |
| | 1.44-1.30 (m, 1 H) |
| I-265 | MS m/z 440.5 [M + H]⁺; ¹H NMR (METHANOL-d4) δ: 9.53-9.79 (m, 1H), |
| | 8.76-9.01 (m, 1H), 7.60 (d, J = 7.9 Hz, 1H), 7.51 (br d, J = 5.4 Hz, 1H), 7.34 (d, |
| | J = 7.9 Hz, 1H), 7.28 (s, 1H), 4.77-4.87 (m, 1H), 3.13-3.23 (m, 1H), 2.97 (q, |
| | J = 10.1 Hz, 1H), 2.49-2.68 (m, 2H), 2.46 (s, 4H), 2.12-2.33 (m, 1H). NH and |
| | OH not observed. |
| I-267 | MS m/z 380.3 [M + H]⁺; ¹H NMR (400 MHz, METHANOL-d4) δ ppm 9.56 (s, |
| | 1 H) 8.70 (br d, J = 5.38 Hz, 1 H) 7.25 (d, J = 5.50 Hz, 1 H) 6.40 (d, J = 14.51 Hz, |
| | 2 H) 4.45-4.66 (m, 1 H) 3.54 (s, 3 H) 3.35-3.49 (m, 1 H) 2.94-3.09 (m, 1 H) |
| | 2.58-2.70 (m, 2 H) 2.28 (s, 3 H) 2.04-2.15 (m, 1 H) 1.90-2.01 (m, 1 H) |
| | 1.64-1.86 (m, 2 H) |
| I-268 | MS m/z 366.1 [M + H]⁺; ¹H NMR (400 MHz, METHANOL-d4) δ 8.40 (br s, |
| | 1H, formic acid peak), 9.56 (s, 1 H) 8.73 (br d, J = 5.38 Hz, 1 H) 7.35 (d, J = 5.63 |
| | Hz, 1 H) 6.26 (s, 2 H) 4.60-4.71 (m, 1 H) 3.52-3.75 (m, 1 H) 2.91-3.08 (m, |
| | 1 H) 2.65-2.87 (m, 2 H) 2.20 (s, 3 H) 2.06-2.15 (m, 1 H) 1.88-2.00 (m, 1 H) |
| | 1.63-1.84 (m, 2 H). NH and OH not observed |
| I-287 | MS m/z 400.3 [M + H]⁻; ¹H NMR (400 MHz, DMSO-d6) d: 9.81(s, 1H), 8.86(d, |
| | J = 4.0 Hz, 1H), 7.67(d, J = 8.0 Hz, 1H), 7.45-7.43(m, 1H), 7.32-7.31(m, 1H), |
| | 7.28-7.26(m, 1H), 7.24-7.23(m, 1H), 6.85(t, J = 110.0 Hz, 1H), 4.49-4.46(m, |
| | 1H), 3.91(s, 3H), 3.12-3.09(m, 1H), 2.75-2.72(m, 1H), 2.23(s, 3H), |
| | 2.04-1.91(m, 3H), 1.77-1.75(m, 1H), 1.64-1.60(m, 1H), 1.50-1.45(m, 1H). |
| I-290 | MS m/z 400.0 [M − H]⁻; ¹H NMR (400 MHz, METHANOL-d4) δ = 9.77 (s, 1H), |
| | 8.87 (d, J = 5.6 Hz, 1H), 7.76 (s, 1H), 7.70 (br d, J = 8.0 Hz, 1H), 7.55 (d, J = |
| | 7.9 Hz, 1H), 7.28 (d, J = 5.6 Hz, 1H), 4.66 (td, J = 4.4, 8.4 Hz, 1H), 3.15 (br s, |
| | 1H), 2.72 (br s, 1H), 2.38 (s, 5H), 2.23-2.19 (m, 3H), 2.16-2.04 (m, 1H), |
| | 1.97-1.86 (m, 1H), 1.85-1.62 (m, 2H); NH wasn′t observed |
| I-291 | MS m/z 446.8 [M + H]⁺; ¹H NMR (400 MHz, METHANOL-d4) δ = 9.73 (s, 1H), |
| | 8.85 (d, J = 5.6 Hz, 1H), 7.62 (d, J = 7.8 Hz, 1H), 7.47-7.41 (m, 2H), |
| | 7.41-7.36 (m, 1H), 4.78-4.60 (m, 2H), 3.15 (br s, 1H), 2.72 (br s, 1H), 2.45-2.24 |
| | (m, 5H), 2.11 (br s, 1H), 1.98-1.85 (m, 1H), 1.85-1.60 (m, 2H), 1.19-1.06 |
| | (m, 6H); NH wasn′t observed |
| I-296 | MS m/z 392.5 [M + H]⁺; ¹H NMR (METHANOL-d4) δ: 9.73 (s, 1H), 8.84 (d, |
| | J = 5.8 Hz, 1H), 7.30 (d, J = 5.5 Hz, 1H), 7.19 (d, J = 8.3 Hz, 1H), 7.07 (s, 1H), |
| | 6.94 (dd, J = 8.5, 1.9 Hz, 1H), 4.60-4.71 (m, 1H), 3.90 (s, 3H), 3.10-3.26 (m, |
| | 1H), 2.65-2.85 (m, 1H), 2.54-2.65 (m, 1H), 2.41 (s, 3H), 2.30-2.38 (m, 1H), |
| | 2.03-2.21 (m, 1H), 1.59-1.95 (m, 3H), 1.16 (d, J = 6.7 Hz, 3H), 1.08 (d, J = 6.7 |
| | Hz, 3H). NH and OH not observed. |
| I-299 | MS m/z 384.4 [M + H]⁺; ¹H NMR (400 MHz, METHANOL-d4) δ 9.73 (s, 1 H), |
| | 8.89 (br d, J = 5.63 Hz, 1 H), 8.40 (br s, 1 H, formic acid), 7.69 (s, 1 H), 7.54 (br |
| | d, J = 7.88 Hz, 1 H), 7.37 (d, J = 6.50 Hz, 2 H), 6.69 (t, J = 54.66 Hz, 1 H), |
| | 4.79-4.65 (m, 1 H), 3.93-3.73 (m, 1 H), 3.50-3.35 (m, 1 H), 3.15-2.95 (m, 2 H), |
| | 2.89 (s, 3 H), 2.54 (s, 3 H), 2.33-2.12 (m, 2 H), 2.02-1.84 (m, 2 H). 1H not |
| | observed (NH) |
| I-316 | MS m/z 391.4 [M + H]⁺; ¹H NMR (400 MHz, CD₃OD) δ 9.72 (s, 1H), 8.87 (s, |
| | 1H), 7.69-7.42 (m, 2H), 7.39-7.16 (m, 2H), 4.85-4.75 (m, 1H), 2.74-2.60 |
| | (m, 2H), 2.37-2.20 (m, 2H), 1.45 (s, 3H), NH and 2 OHs not observed. |
| I-319 | MS m/z 454.3 [M + H]⁺; ¹H NMR (400 MHz, Chloroform-d) δ 9.52 (s, 1H), 8.94 |
| | (d, J = 5.6 Hz, 1H), 8.07 (s, 1H), 7.91 (d, J = 8.4 Hz, 1H), 7.67 (d, J = 5.6 Hz, 1H), |
| | 7.51 (d, J = 8.4 Hz, 1H), 6.98 (t, J = 54.8 Hz, 1H), 6.66 (s, 1H), 4.81-4.80 (m, 1H), |
| | 2.82-2.80 (m, 2H), 2.55-2.52 (m, 1H), 2.35 (s, 3H), 2.20-2.10 (m, 2H), |
| | 1.91-1.81 (m, 1H), 1.67-1.63 (m, 2H). |
| I-320 | MS m/z 454.3 [M + H]⁺; ¹H NMR (400 MHz, Chloroform-d) d: 9.55 (s, 1H), |
| | 8.88 (d, J = 5.6 Hz, 1H), 7.72 (s, 1H), 7.46 (s, 2H), 7.31 (d, J = 5.6 Hz, 1H), |
| | 6.95 (t, J = 55.2 Hz, 1H), 6. 81 (s, 1H), 4.83-4.82 (m, 1H), 2.89-2.83(m, |
| | 2H), 2.56-2.53 (m, 1H), 2.37 (s, 3H), 2.22-2.07 (m, 2H), 1.92-1.89 (m, |
| | 1H), 1.69-1.68 (m, 2H). |

### Example 3

### Preparation of Compound I-10

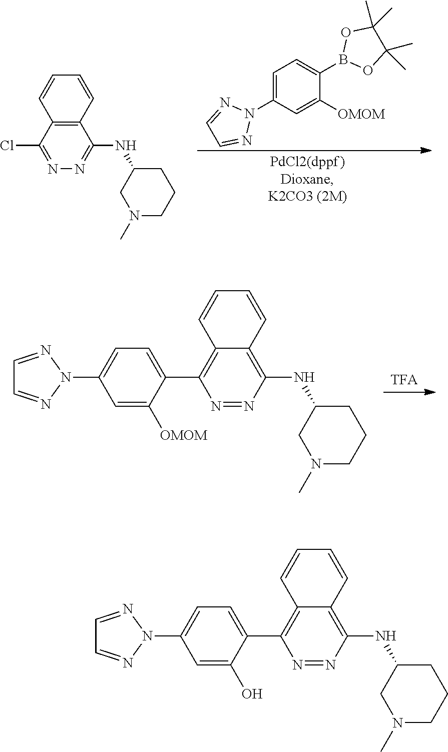

### Step 1. (R)-4-(2-(Methoxymethoxy)-4-(2H-1,2,3-triazol-2-yl)phenyl)-N-(1-methylpiperidin-3-yl)phthalazin-1-amine

To a solution of (R)-4-chloro-N-(1-methylpiperidin-3-yl)phthalazin-1-amine (Intermediate 19a, 100 mg, 0.36 mmol) in 1.5 mL 1,4-dioxane and 0.3 mL water was added 2-(3-(methoxymethoxy)-4-(4,4,5,5-tetramethyl-1,3,2-dioxaborolan-2-yl)phenyl)-2H-1,2,3-triazole (119 mg, 0.36 mmol, 1.0 eq), K₂CO₃ (100 mg, 0. 72 mmol, 2.0 eq) and Pd(dppf)Cl₂ (40 mg, 0.15 eq). This mixture was stirred at 90° C. under N₂ for 16 h before concentration. The residue was purified by flash column chromatography (DCM/MeOH=17:3) to give (R)-4-(2-(methoxymethoxy)-4-(2H-1,2,3-triazol-2-yl)phenyl)-N-(1-methylpiperidin-3-yl)phthalazin-1-amine (124 mg, 76%) as brown oil. MS m/z 446.2 [M+H]⁺.

### Step 2. (R)-2-(4-((1-Methylpiperidin-3-yl)amino)phthalazin-1-yl)-5-(2H-1,2,3-triazol-2-yl)phenol

A solution of (R)-4-(2-(methoxymethoxy)-4-(2H-1,2,3-triazol-2-yl)phenyl)-N-(1-methylpiperidin-3-yl)phthalazin-1-amine (124 mg, 0.28 mmol) in 1 mL TFA was stirred at 25° C. for 3 h before concentration. The residue was mixed with 5 mL water, basified with sat. NaHCO₃ to pH=8, and extracted with DCM (10 mL/3). The combined DCM was dried over Na₂SO₄, concentrated, and purified by flash column chromatography [DCM/NH₃ (7 M in MeOH)=10:1] to give (R)-2-(4-((1-methylpiperidin-3-yl)amino)phthalazin-1-yl)-5-(2H-1,2,3-triazol-2-yl)phenol (74 mg, 72%) as brown foam. MS m/z 402.2 [M+H]⁺; ¹H NMR (DMSO-d₆) δ: 10.28 (s, 1H), 8.40 (d, J=8.2 Hz, 1H), 8.16 (s, 2H), 7.86 (t, J=7.6 Hz, 1H), 7.79 (t, J=7.2 Hz, 1H), 7.72 (d, J=2.1 Hz, 1H), 7.63 (dd, J=8.2, 2.1 Hz, 1H), 7.54 (d, J=8.1 Hz, 1H), 7.47 (d, J=8.3 Hz, 1H), 7.10 (d, J=7.7 Hz, 1H), 4.52-4.36 (m, 1H), 3.19-3.03 (m, 1H), 2.81-2.69 (m, 1H), 2.24 (s, 3H), 2.04-1.89 (m, 3H), 1.83-1.72 (m, 1H), 1.70-1.56 (m, 1H), 1.53-1.42 (m, 1H).

### Example 4

### Preparation of Compound I-15

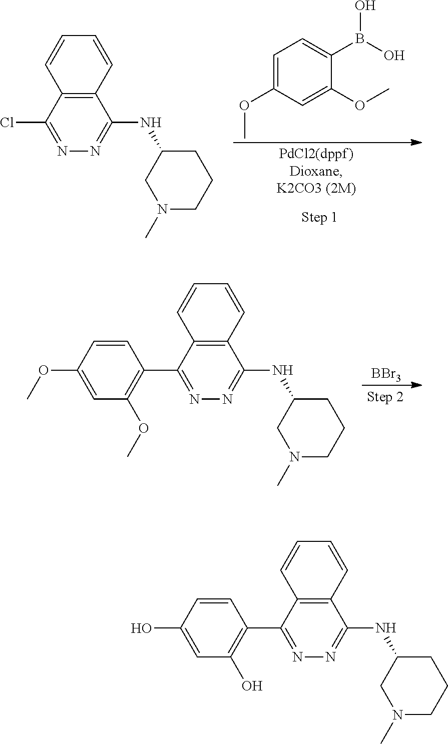 [SMILES: CN1CCC[C@@H](Nc2nnc(Cl)c3ccccc23)C1 ; COc1ccc(B(O)O)c(OC)c1 ; COc1ccc(-c2nnc(N[C@@H]3CCCN(C)C3)c3ccccc23)c(OC)c1 ; "BBr3" ; CN1CCC[C@@H](Nc2nnc(-c3ccc(O)cc3O)c3ccccc23)C1]

### Step 1. (R)-4-(2,4-Dimethoxyphenyl)-N-(1-methylpiperidin-3-yl)phthalazin-1-amine

To a solution of (R)-4-chloro-N-(1-methylpiperidin-3-yl)phthalazin-1-amine (Intermediate 19a, 185 mg, 0.67 mmol) in 1 mL 1,4-dioxane and 0.2 mL water was added (2,4-dimethoxyphenyl)boronic acid (182 mg, 1.0 mmol, 1.5 eq), K₂CO₃ (185 mg, 1.34 mmol, 2.0 eq) and Pd(dppf)Cl₂(73 mg, 0.1 mmol, 0.15 eq). This mixture was stirred at 90° C. under N₂ for 16 h before concentration. The residue was purified by flash column chromatography (DCM/MeOH=10:1) to give (R)-4-(2,4-dimethoxyphenyl)-N-(1-methylpiperidin-3-yl)phthalazin-1-amine (200 mg, 79%) as brown solid. MS m/z 379.1 [M+H]⁺.

### Step 2. (R)-4-(4-((1-Methylpiperidin-3-yl)amino)phthalazin-1-yl)benzene-1,3-diol

To a solution of (R)-4-(2,4-dimethoxyphenyl)-N-(1-methylpiperidin-3-yl)phthalazin-1-amine (120 mg, 0.31 mmol) in 1.5 mL DCM was added BBr₃ (1 M in DCM, 2.2 mL, 2.2 mmol, 7 eq). The mixture was stirred at RT for 3 h then diluted with MeOH (3 ml), stirred for another 30 min and concentrated. The residue was purified by flash column chromatography [DCM/NH₃(7 M in MeOH)=20:1] and prep-TLC (DCM/Et₃N (0.07 M in MeOH)) to give (R)-4-(4-((1-methylpiperidin-3-yl)amino)phthalazin-1-yl)benzene-1,3-diol (45 mg, 40%) as white solid. MS m/z 351.1 [M+H]⁺; ¹H NMR (MeOD-d4) δ: 8.25 (d, J=8.1 Hz, 1H), 7.86-7.81 (m, 1H), 7.80-7.72 (m, 2H), 7.21-7.14 (m, 1H), 6.48-6.46 (m, 2H), 4.62-4.48 (m, 1H), 3.47 (q, J=7.3 Hz, 1H), 3.15-3.05 (m, 1H), 2.73-2.60 (m, 1H), 2.40-2.60 (m, 4H), 2.08-1.98 (m, 1H), 1.90-1.80 (m, 1H), 1.78-1.68 (m, 1H), 1.68-1.55 (m, 1H).

### Example 5

### Preparation of Compound I-71

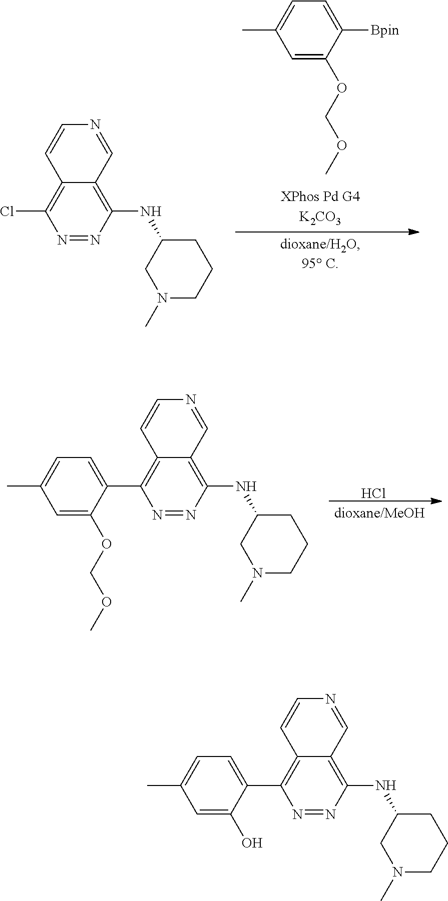

### Step 1: 1-[2-(Methoxymethoxy)-4-methyl-phenyl]-N-[(3R)-1-methyl-3-piperidyl]pyrido[3,4-d]pyridazin-4-amine

1-Chloro-N-[(3R)-1-methyl-3-piperidyl]pyrido[3,4-d]pyridazin-4-amine (Intermediate 20a, 0.50 g, 1.80 mmol), [2-(methoxymethoxy)-4-methyl-phenyl]boronic acid (prepared according to the procedure of Intermediate 5a, 0.46 g, 2.34 mmol), and XPhos Pd G4 (0.098 g, 0.108 mmol) were added to a vial and evacuated and refilled with Ar. The mixture was dissolved in dioxane (9.0 mL) and K₂CO₃ (2 M, 2.7 mL, 5.40 mmol). The mixture was sparged with Ar for 5 min. The reaction was then heated to 95° C. for 2.5 h. The mixture was diluted with EtOAc and filtered through Celite. The filtrate was washed with brine and concentrated. Purification by chromatography on SiO₂ (1M NH₄OH in MeOH:DCM, 0 to 10%) gave an dark-yellow foam (0.596 g, 80%). MS m/z 394.1 [M+H]⁺.

### Step 2. 5-Methyl-2-[4-[[(3R)-1-methyl-3-piperidyl]amino]pyrido[3,4-d]pyridazin-1-yl]phenol

1-[2-(Methoxymethoxy)-4-methyl-phenyl]-N-[(3R)-1-methyl-3-piperidyl]pyrido[3,4-d]pyridazin-4-amine (1.19 g, 3.02 mmol) in MeOH (2 mL) was added HCl/dioxane (4M, 4.0 mL) and stirred for 2 h. The reaction was concentrated and then dissolved in DCM/iPrOH (9:1). The solution was washed with sat. NaHCO₃, brine, dried (Na₂SO₄), filtered and concentrated to give a light-yellow solid (0.905 g, 86%). MS m/z 350.4 [M+H]⁺; ¹H NMR (400 MHz, DMSO-d₆) δ 9.76 (s, 1H), 9.61 (s, 1H), 8.83 (d, J=5.5 Hz, 1H), 7.52 (d, J=7.5 Hz, 1H), 7.30 (d, J=5.6 Hz, 1H), 7.19 (d, J=7.5 Hz, 1H), 6.81 (s, 1H), 6.79 (d, J=7.8 Hz, 1H), 4.52-4.34 (m, 1H), 3.10 (d, J=7.9 Hz, 1H), 2.72 (d, J=10.4 Hz, 1H), 2.33 (s, 3H), 2.22 (s, 3H), 2.07-1.98 (m, 1H), 1.97-1.86 (m, 2H), 1.82-1.72 (m, 1H), 1.68-1.54 (m, 1H), 1.52-1.39 (m, 1H).

The compounds below were prepared according to the procedure of Example 5 by substituting the appropriate starting materials, reagents and reaction conditions.

| Compound | Spectral Data |
|---|---|
| I-14 | MS m/z 353.1 [M + H]⁺; ¹H NMR (DMSO-d6) δ: 9.91 (s, 1H), 8.41 (d, J = 8.3 |
| | Hz, 1H), 7.86 (t, J = 7.6 Hz, 1H), 7.78 (t, J = 7.6 Hz, 1H), 7.34 (q, J = 7.8, 7.3 |
| | Hz, 2H), 7.12 (d, J = 7.7 Hz, 1H), 6.85 (d, J = 8.2 Hz, 1H), 6.79 (t, J = 8.7 Hz, |
| | 1H), 4.49-4.37 (m, 1H), 3.16-3.02 (m, 1H), 2.72 (d, J = 10.9 Hz, 1H), 2.23 |
| | (s, 3H), 1.96 (q, J = 10.8, 9.4 Hz, 3H), 1.76 (dd, J = 10.3, 6.0 Hz, 1H), 1.62 (q, |
| | J = 12.3 Hz, 1H), 1.48 (tt, J = 13.2, 6.4 Hz, 1H). |
| I-16 | MS m/z 353.1 [M + H]⁺; ¹H NMR (DMSO-d6) δ: 10.17 (s, 1H), 8.38 (d, J = 8.1 |
| | Hz, 1H), 7.84 (t, J = 7.4 Hz, 1H), 7.82-7.74 (m, 1H), 7.47 (d, J = 8.2 Hz, 1H), |
| | 7.34-7.28 (m, 1H), 7.11-7.03 (m, 1H), 6.83-6.75 (m, 2H), 4.45-4.35 (m, |
| | 1H), 3.15-3.05 (m, 1H), 2.78-2.74 (m, 1H), 2.22 (s, 3H), 2.05-1.90 (m, |
| | 3H), 1.80-1.73 (m, 1H), 1.65-1.57 (m, 1H), 1.53-1.45 (m, 1H). |
| I-17 | MS m/z 349.0 [M + H]⁺; ¹H NMR (400 MHz, MeOD-d4) δ 8.28 (d, J = 8.0 Hz, |
| | 1H), 7.86 (t, J = 7.6 Hz, 1H), 7.78 (t, J = 7.6 Hz, 1H), 7.67 (d, J = 8.4 Hz, 1H), |
| | 7.20-7.10 (m, 2H), 6.87 (d, J = 8.0 Hz, 1H), 4.64-4.53 (m, 1H), 3.27-3.04 |
| | (m, 2H), 2.85-2.68 (m, 1H), 2.50-2.35 (m, 4H), 2.32 (s, 3H), 2.13-2.00 (m, |
| | 1H), 1.97-1.85 (m, 1H), 1.86-1.74 (m, 1H), 1.73-1.59 (m, 1H), NH and |
| | OH not observed. |
| I-18 | MS m/z 367.2 [M + H]⁺; ¹H NMR (400 MHz, MeOD-d4) δ: 8.27 (d, J = 8.0 Hz, |
| | 1H), 7.90-7.74 (m, 2H), 7.68 (d, J = 8.0 Hz, 1H), 7.02 (d, J = 8.0 Hz, 1H), |
| | 6.82 (d, J = 8.0 Hz, 1H), 4.61-4.52 (m, 1H), 3.26-3.06 (m, 1H), 2.73 (s, 1H), |
| | 2.39 (s, 3H), 3.35-2.15(m, 2H), 2.30 (t, J = 1.6 Hz, 3H), 2.13-1.99 (m, 1H), |
| | 1.93-1.84 (m, 1H), 1.83-1.64 (m, 2H), NH and OH not observed. |
| I-24 | MS m/z 360.3 [M + H]⁺; 1H NMR (400 MHz, DMSO-d6) δ 10.46 (d, J = 4.6 Hz, |
| | 1H), 8.40 (d, J = 8.0 Hz, 1H), 7.89-7.75 (m, 2H), 7.50 (d, J = 7.6 Hz, 1H), |
| | 7.45-7.39 (m, 2H), 7.33 (d, J = 1.2 Hz, 1H), 7.16 (d, J = 7.6 Hz, 1H), 4.49-4.36 |
| | (m, 1H), 3.09 (d, J = 7.6 Hz, 1H), 2.72 (d, J = 10.0 Hz, 1H), 2.22 (s, 3H), |
| | 2.02-1.88 (m, 3H), 1.80-1.70 (m, 1H), 1.67-1.54 (m, 1H), 1.53-1.40 (m, |
| | 1H). |
| I-25 | MS m/z 349.0 [M + H]⁺; ¹H NMR (400 MHz, MeOD-d4) δ 8.28 (d, J = 8.0 Hz, |
| | 1H), 7.89-7.82 (m, 1H), 7.81-7.75 (m, 1H), 7.70 (d, J = 7.6 Hz, 1H), 7.22 (d, |
| | J = 7.6 Hz, 1H), 6.87-6.80 (m, 2H), 4.62-4.53 (m, 1H), 3.35-3.18 (m, 1H), |
| | 3.24-3.08 (m, 2H), 2.80-2.71 (m, 1H), 2.42 (s, 3H), 2.38 (s, 3H), 2.11-2.01 |
| | (m, 1H), 1.95-1.86 (m, 1H), 1.85-1.76 (m, 1H), 1.73-1.62 (m, 1H), NH |
| | and OH not observed. |
| I-27 | MS m/z 349.1 [M + H]⁺; ¹H NMR (METHANOL-d₄) δ: 8.31 (d, J = 8.0 Hz, 1H), |
| | 7.88 (ddd, J = 8.2, 5.0, 3.4 Hz, 1H), 7.78-7.84 (m, 2H), 7.16-7.31 (m, 2H), 6.92 |
| | (t, J = 7.5 Hz, 1H), 4.56 (dt, J = 8.3, 4.4 Hz, 1H), 3.01-3.24 (m, 1H), 2.69 (br dd, |
| | J = 3.5, 1.5 Hz, 1H), 2.27-2.45 (m, 8H), 1.98-2.11 (m, 1H), 1.83-1.93 (m, 1H), |
| | 1.64-1.83 (m, 2H), NH and OH not observed. |
| I-28 | MS m/z 353.1 [M + H]⁺; ¹H NMR (METHANOL-d₄) δ: 8.29 (d, J = 8.2 Hz, 1H), |
| | 7.87 (t, J = 7.3 Hz, 1H), 7.76-7.82 (m, 1H), 7.63 (d, J = 8.0 Hz, 1H), 7.24 (ddd, |
| | J = 10.9, 8.3, 1.6 Hz, 1H), 7.14 (d, J = 7.3 Hz, 1H), 6.97 (td, J = 7.9, 4.8 Hz, 1H), |
| | 4.59 (dt, J = 8.6, 4.4 Hz, 1H), 3.13-3.28 (m, 1H), 2.66-2.88 (m, 1H), 2.35-2.49 |
| | (m, 5H), 2.00-2.12 (m, 1H), 1.85-1.96 (m, 1H), 1.64-1.83 (m, 2H), NH and OH |
| | not observed. |
| I-50 | MS m/z 349.0 [M + H]⁺; ¹H NMR (400 MHz, CD₃OD) δ 8.30 (d, J = 8.3 Hz, |
| | 1H), 7.87 (t, 7.2 Hz, 1H), 7.76 (t, J = 7.2 Hz, 1H), 7.44 (d, J = 8.1 Hz, 1H), 7.24 |
| | (t, J = 7.9 Hz, 1H), 6.87 (d, J = 7.6 Hz, 1H), 6.81 (d, J = 8.1 Hz, 1H), 4.63-4.60 |
| | (m, 1H), 3.30-3.25 (m, 1H), 2.88-2.84 (m, 1H), 2.48-2.40 (m, 5H), 2.12-2.08 |
| | (m, 1H), 1.97-1.92 (m, 4H), 1.87-1.63 (m, 2H), NH and OH not observed. |
| I-51 | MS m/z 367.0 [M + H]⁺; ¹H NMR (METHANOL-d₄) δ: 8.27 (d, J = 8.3 Hz, 1H), |
| | 7.85 (t, J = 7.2 Hz, 1H), 7.76-7.81 (m, 1H), 7.64 (d, J = 8.0 Hz, 1H), 7.02 (dd, |
| | J = 7.8, 1.3 Hz, 1H), 6.84 (t, J = 7.4 Hz, 1H), 4.59 (dt, J = 8.7, 4.5 Hz, 1H), |
| | 3.24-3.29 (m, 1H), 2.74-2.91 (m, 1H), 2.45 (m, 5H), 2.35 (d, J = 2.0 Hz, 3H), 2.07 (m, |
| | 1H), 1.86-1.98 (m, 1H), 1.67-1.86 (m, 2H), NH and OH not observed. |
| I-41 | MS m/z 363.2 [M + H]⁺; ¹H NMR (400 MHz, MeOD-d4) δ 8.25 (d, 1H), 7.86-7.80 |
| | (m, 1H), 7.78-7.72 (m, 1H), 7.69 (d, J = 8.0 Hz, 1H), 7.08 (s, 1H), 6.78 |
| | (s, 1H), 4.62-4.50 (m, 1H), 3.21-3.07 (m, 1H), 2.77-2.63 (m, 1H), 2.37 (m, |
| | 5H), 2.29 (s, 3H), 2.23 (s, 3H), 2.09-2.02 (m, 1H), 1.93-1.83 (m, 1H), |
| | 1.82-1.57 (m, 2H), NH and OH not observed. |
| I-42 | MS m/z 363.2 [M + H]⁺; ¹H NMR (400 MHz, DMSO-d6) δ 10.09 (s, 1H), 8.41 |
| | (d, J = 8.0 Hz, 1H), 7.98-7.66 (m, 3H), 7.14 (d, J = 7.6 Hz, 2H), 6.82 (d, J = |
| | 7.6 Hz, 1H), 4.49-4.36 (m, 1H), 3.12-3.04 (m, 1H), 2.76-2.66 (m, 1H), |
| | 2.30 (s, 3H), 2.21 (s, 3H), 2.18 (s, 3H), 2.06-1.85 (m, 3H), 1.80-1.71 (m, |
| | 1H), 1.68-1.56 (m, 1H), 1.53-1.43 (m, 1H). |
| I-56 | MS m/z 417.0 [M + H]⁺; ¹H NMR (400 MHz, CDCl₃) δ: 8.37-8.24 (m, 1H), |
| | 8.03 (d, J = 8.0 Hz, 1H), 7.90-7.78 (m, 2H), 7.58 (d, J = 8.0 Hz, 1H), 7.21 (d, |
| | J = 1.6 Hz, 1H), 7.08-7.03 (m, 1H), 6.30-6.22 (m, 1H), 4.82-4.61 (m, 1H), |
| | 4.45-4.27 (m, 2H), 3.96 (t, J = 8.0 Hz, 2H), 2.90-2.61 (s, 2H), 2.60-2.48 |
| | (m, 2H), 2.33 (s, 3H), 2.20-2.03 (m, 2H), 1.90-1.76 (m, 3H), 1.71-1.55 (m, |
| | 3H). |
| I-57 | MS m/z 403.2 [M + H]⁺; ¹H NMR (400 MHz, CDCl₃) δ: 8.27 (d, J = 8.0 Hz, |
| | 1H), 8.07 (d, J = 8.0 Hz, 1H), 7.91-7.83 (td, J = 12.0, 4.0 Hz, 1H), 7.81 (td, J = |
| | 12.0, 4.0 Hz, 1H), 7.59 (d, J = 8.0 Hz, 1H), 7.14 (d, J = 1.6 Hz, 1H), 6.99 (dd, |
| | J = 8.0, 1.6 Hz, 1H), 6.40 (s, 1H), 6.35-6.29 (m, 1H), 5.05 (td, J = 4.8, 2.0 Hz, |
| | 2H), 4.88 (m, 2H), 4.76-4.68 (m, 1H), 2.95-2.75 (m, 2H), 2.51 (m, 1H), 2.34 |
| | (s, 3H), 2.13 (m, 2H), 1.84 (m, 1H), 1.70-1.51 (m, 2H). |
| I-61 | MS m/z 405.1 [M + H]⁺; ¹H NMR (400 MHz, MeOD-d4) δ 8.27 (d, J = 8.0 Hz, |
| | 1H), 7.86 (t, J = 7.6 Hz, 1H), 7.79 (t, J = 7.6 Hz, 1H), 7.67 (d, J = 8.0 Hz, 1H), |
| | 7.28 (d, J = 8.0 Hz, 1H), 6.98-6.89 (m, 2H), 4.72-4.60 (m, 1H), 4.17-4.02 |
| | (m, 2H), 3.98-3.87 (m, 1H), 3.76 (t, J = 7.6 Hz, 1H), 3.57-3.38 (m, 2H), |
| | 3.09-2.97 (m, 1H), 2.87-2.69 (m, 2H), 2.60 (s, 3H), 2.46-2.37 (m, 1H), |
| | 2.20-2.10 (m, 1H), 2.10-1.97 (m, 2H), 1.88-1.69 (m, 2H). |
| I-72 | MS m/z 354.2 [M + H]⁺; ¹H NMR (400 MHz, DMSO-d6) δ 9.81 (s, 1H), 8.86 (d, |
| | J = 5.6 Hz, 1H), 8.21 (s, 1H), 7.77-7.63 (m, 1H), 7.37 (dd, J = 15.2, 8.4 Hz, |
| | 1H), 7.18 (d, J = 5.6 Hz, 1H), 6.92-6.76 (m, 2H), 4.56-4.45 (m, 1H), |
| | 3.23-3.12 (m, 1H), 2.86-2.78 (m, 1H), 2.31 (s, 3H), 2.15-1.97 (m, 3H), |
| | 1.88-1.76 (m, 1H), 1.70-1.60 (m, 1H), 1.57-1.44 (m, 1H). |
| I-74 | MS m/z 361.2 [M + H]⁺. ¹H NMR (400 MHz, DMSO-d6) δ 8.38 (d, J = 8.0 Hz, |
| | 1H), 8.33 (s, 1H), 7.85 (t, J = 7.2 Hz, 1H), 7.79 (t, J = 7.2 Hz, 1H), 7.50 (d, J = |
| | 7.2 Hz, 1H), 7.26 (s, 1H), 7.16 (s, 1H), 4.55-4.43 (m, 1H), 3.31-3.20 (m, |
| | 1H), 2.95-2.85 (m, 1H), 2.41 (s, 3H), 2.31-2.19 (m, 2H), 2.03-1.95 (m, |
| | 1H), 1.88-1.77 (m, 1H), 1.73-1.46 (m, 2H). |
| I-75 | MS m/z 404.0 [M + H]⁺; ¹H NMR (400 MHz, MeOD-d4) δ 8.13 (d, J = 7.2 Hz, |
| | 1H), 7.77-7.64 (m, 3H), 7.52 (d, J = 7.2 Hz, 1H), 7.11 (d, J = 7.2 Hz, 1H), |
| | 4.55-4.36 (m, 1H), 3.18-3.07 (m, 1H), 2.81-2.64 (m, 1H), 2.50-2.42 (m, |
| | 1H), 2.38 (s, 3H), 2.00-1.90 (m, 1H), 1.89-1.76 (m, 1H), 1.76-1.53 (m, |
| | 2H), 1.27-1.12 (m, 1H). |
| I-78 | MS m/z 351.0 [M + H]⁺; ¹H NMR (400 MHz, DMSO-d6) δ: 11.99 (s, 1H), |
| | 9.74 (s, 1H), 8.83 (d, J = 4.0 Hz, 1H), 7.63-7.53 (m, 2H), 7.42-7.36 (m, 1H), |
| | 6.23-6.17 (m, 1H), 4.51-4.34 (m, 1H), 3.14-3.04 (m, 1H), 2.80-2.69 (m, |
| | 1H), 2.29 (s, 3H), 2.22 (s, 3H), 2.05-1.89 (m, 3H), 1.82-1.72 (m, 1H), |
| | 1.69-1.54 (m, 1H), 1.52-1.39 (m, 1H). |
| I-79 | MS m/z 404.0 [M + H]⁺; ¹H NMR (400 MHz, MeOD-d4) δ 8.24 (d, J = 8.4 Hz, |
| | 1H), 8.19 (s, 1H), 7.87-7.77 (m, 2H), 7.73-7.69 (m, 1H), 7.01 (s, 1H), |
| | 4.76-4.64 (m, 1H), 3.63 (s, 1H), 3.19 (s, 1H), 2.95-2.80 (m, 2H), 2.70 (s, 3H), |
| | 2.25-2.16 (m, 1H), 2.12-2.03 (m, 1H), 2.00-1.88 (m, 1H), 1.84-1.72 (m, 1H). |
| I-80 | MS m/z 361.2 [M + H]⁺; ¹H NMR (400 MHz, MeOD-d4) δ 8.12 (d, J = 6.4 Hz, |
| | 1H), 7.76-7.66 (m, 3H), 7.63-7.57 (m, 1H), 7.15 (d, J = 7.2 Hz, 1H), |
| | 4.64-4.56 (m, 1H), 3.40-3.32 (m, 1H), 3.03-2.93 (m, 1H), 2.90-2.68 (m, 2H), |
| | 2.59 (s, 3H), 2.08-1.94 (m, 2H), 1.86-1.68 (m, 2H); |
| I-82 | MS m/z 350.4 [M + H]⁺; ¹H NMR (400 MHz, CDCl₃) δ 9.55 (s, 1H), 8.92 (d, J = |
| | 5.7 Hz, 1H), 7.99 (d, J = 5.7 Hz, 1H), 7.36 (d, J = 7.8 Hz, 1H), 7.24-7.14 (m, |
| | 2H), 6.91 (t, J = 7.6 Hz, 1H), 6.80 (s, 1H), 4.79-4.68 (m, 1H), 3.03-2.77 (m, |
| | 2H), 2.70-2.54 (m, 1H), 2.42 (s, 3H), 2.37 (s, 3H), 2.30-2.21 (m, 1H), |
| | 2.16-2.01 (m, 1H), 1.99-1.87 (m, 1H), 1.74-1.61 (m, 2H). |
| I-86 | MS m/z 350.2 [M + H]⁺; 1H NMR (400 MHz, CD₃OD) δ 9.10 (dd, J = 4.4, 1.7 |
| | Hz, 1H), 8.50 (s, 1H), 8.09 (dd, J = 8.3, 1.7 Hz, 1H), 7.82 (dd, J = 8.5, 4.4 Hz, |
| | 1H), 7.27 (d, J = 7.7 Hz, 1H), 6.89-6.81 (m, 2H), 4.64-4.50 (m, 1H), |
| | 3.67-3.49 (m, 1H), 3.24-3.08 (m, 1H), 3.02-2.78 (m, 2H), 2.73 (s, 3H), 2.39 (s, |
| | 3H), 2.25-2.02 (m, 2H), 1.98-1.74 (m, 2H) |
| I-88 | MS m/z 368.0 [M + H]⁺; ¹H NMR (400 MHz, MeOD-d4) δ 9.65 (s, 1H), 8.79 (d, |
| | J = 5.7 Hz, 1H), 7.53 (d, J = 5.6 Hz, 1H), 7.02 (d, J = 9.7 Hz, 1H), 6.78 (d, J = |
| | 6.5 Hz, 1H), 4.62-4.51 (m, 1H), 3.19-2.99 (m, 1H), 2.71-2.58 (m, 1H), |
| | 2.32 (s, 3H), 2.29-2.13 (m, 5H), 2.08-2.00 (m, 1H), 1.89-1.80 (m, 1H), |
| | 1.78-1.54 (m, 2H). |
| I-94 | MS m/z 414.0 [M + H]⁺; 1H NMR (400 MHz, DMSO-d6) δ: 8.38 (d, J = 8.0 Hz, |
| | 1H), 7.85 (ddd, J = 8.3, 7.0, 1.4 Hz, 1H), 7.79 (ddd, J = 8.3, 7.0, 1.2 Hz, 1H), |
| | 7.63 (dd, J = 8.0, 1.3 Hz, 1H), 7.27-7.21 (m, 1H), 7.17 (t, J = 2.0 Hz, 1H), |
| | 7.12-7.06 (m, 2H), 6.77 (t, J = 2.4 Hz, 1H), 6.38 (dd, J = 2.8, 1.8 Hz, 1H), |
| | 4.49-4.38 (m, 1H), 3.18-3.11 (m, 1H), 2.82-2.72 (m, 1H), 2.28 (s, 3H), |
| | 2.09-1.94 (m, 3H), 1.83-1.74 (m, 1H), 1.70-1.57 (m, 1H), 1.55-1.43 (m, |
| | 1H). |
| I-92 | MS m/z 360.1 [M + H]⁺; ¹H NMR (400 MHz, CD₃OD) δ 9.60 (s, 1H), 8.74 (d, J = |
| | 5.7 Hz, 1H), 7.45 (d, J = 7.8 Hz, 1H), 7.36 (dd, J = 5.7 Hz, 1H), 7.25 (dd, J = |
| | 7.8, 1.4 Hz, 1H), 7.18 (d, J = 1.4 Hz, 1H), 4.59-4.46 (m, 1H), 3.15-3.00 (m, |
| | 1H), 2.72-2.59 (m, 1H), 2.34-2.20 (m, 5H), 2.07-2.04 (m, 1H), 1.86-1.76 |
| | (m, 1H), 1.76-1.48 (m, 2H). |
| I-123 | MS m/z 402.3 [M + H]⁺; ¹H NMR (400 MHz, METHANOL-d4) δ: 8.35 (d, J = |
| | 9.38 Hz, 1 H), 8.30 (s, 1 H, formic acid), 7.95 (t, J = 7.63 Hz, 1 H), 7.87 (t, J = |
| | 8.00 Hz, 1 H), 7.68 (d, J = 8.13 Hz, 1 H), 7.30 (d, J = 7.88 Hz, 1 H), 7.18 (s, 1 |
| | H), 7.05 (d, J = 8.00 Hz, 1 H), 4.74-4.63 (m, 1 H), 3.97-3.78 (m, 1 H), |
| | 3.51-3.38 (m, 2 H), 3.19-2.98 (m, 2 H), 2. 90 (s, 3 H), 2.13-2.34 (m, 2 H), |
| | 1.80-2.07 (m, 1 H). 3Hs not observed (3 NH) |
| I-122 | MS m/z 454.2 [M + H]⁺; ¹H NMR (400 MHz, METHANOL-d4) δ: 9.00 (d, J = |
| | 5.63 Hz, 1 H), 8.90 (s, 1 H), 8.38 (s, 1 H, formic acid), 8.26 (d, J = 5.63 Hz, 1 |
| | H), 7.88-7.77 (m 2 H), 7.73 (s, 1 H), 6.96 (td, J = 73.04 Hz, 8.75 Hz, 1 H), |
| | 4.77-4.67 (m, 1 H), 3.87-3.72 (m, 1 H), 3.46-3.37 (m, 1 H), 3.16-2.97 (m, |
| | 2 H), 2.88 (s, 3 H), 2.32-2.12 (m, 2 H), 2.05-1.83 (m, 2 H); 1H not observed |
| | (NH) |
| I-99 | MS m/z 350.2 [M + H]⁺; ¹H NMR (MeOH-d4) d: 8.99 (s, 1H), 8.90 (d, J = 5.5 |
| | Hz, 1H), 8.21 (d, J = 5.5 Hz, 1H), 7.31 (d, J = 7.5 Hz, 1H), 6.90 (d, J = 7.5 Hz, |
| | 1H), 6.86 (s, 1H), 4.51-4.63 (m, 1H), 3.20-3.30 (m, 1H), 2.75-2.85 (m, 1H), |
| | 2.38-2.49 (m, 8H), 2.05-2.15 (m, 1H), 1.90-1.97 (m, 1H), 1.65-1.87 (m, 2H). |
| | Two exchangeable protons are not seen. |
| I-125 | MS m/z 416.2 [M + H]⁺; ¹H NMR (400 MHz, DMSO-d6) δ: 8.42 (d, J = 8.13 |
| | Hz, 1 H), 8.18 (s, 1 H, formic acid), 7.86 (t, J = 8.50 Hz, 1 H), 7.77 (t, J = 7.63 |
| | Hz, 1 H), 7.39 (d, J = 8.25 Hz, 1 H), 7.26 (d, J = 7.63 Hz, 1 H), 7.21-7.13 (m, |
| | 1 H), 7.01 (d, J = 7.75 Hz, 1 H), 6.87 (s, 1 H), 5.48 (d, J = 4.88 Hz, 1 H), 4.54- |
| | 4.40 (m, 1 H), 3.18-3.08 (m, 1 H), 2.83-2.73 (m, 1 H), 2.66 (d, J = 4.63 Hz, |
| | 3 H), 2.26 (s, 3 H), 2.05-1.92 (m, 3 H), 1.84-1.72 (m, 1 H), 1.68-1.55 (m, 1 |
| | H), 1.55-1.40 (m, 1 H). |
| I-102 | MS m/z 420.0 [M + H]⁺; ¹H NMR (400 MHz, DMSO-d6) δ: 10.38 (s, 1H), 9.78 |
| | (s, 1H), 8.85 (d, J = 4.0 Hz, 1H), 7.63 (d, J = 8.0 Hz, 1H), 7.48-7.41 (m, 1H), |
| | 7.29 (dd, J = 5.6, 0.6 Hz, 1H), 7.00-6.92 (m, 2H), 4.53-4.40 (m, 1H), |
| | 3.20-3.08 (m, 1H), 2.84-2.69 (m, 1H), 2.26 (s, 3H), 2.07-1.90 (m, 3H), |
| | 1.84-1.73 (m, 1H), 1.70-1.56 (m, 1H), 1.55-1.39 (m, 1H); |
| I-103 | MS m/z 418.2 [M + H]⁺; ¹H NMR (400 MHz, CD₃OD) δ 9.72 (d, J = 0.7 Hz, |
| | 1H), 8.80 (d, J = 5.6 Hz, 1H), 7.28 (d, J = 5.6 Hz, 1H), 7.20 (s, 1H), 7.08 (s, |
| | 1H), 4.68-4.60 (m, 1H), 3.28-3.16 (m, 1H), 2.84-2.72 (m, 1H), 2.45-2.30 |
| | (m, 5H), 2.15-2.08 (m, 4H), 1.96-1.88 (m, 1H), 1.83-1.68 (m, 2H). |
| I-105 | MS m/z 350.0 [M + H]⁺; ¹H NMR (400 MHz, CD₃OD) δ: 9.70 (s, 1H), 8.77 (t, |
| | J = 14.2 Hz, 1H), 7.28 (d, J = 5.6 Hz, 1H), 7.24 (t, J = 7.9 Hz, 1H), 6.88 (d, J = |
| | 7.6 Hz, 1H), 6.81 (d, J = 8.1 Hz, 1H), 4.70-4.53 (m, 1H ), 3.24-3.09 (m, |
| | 1H), 2.79-2.64 (m, 1H), 2.50-2.23 (m, 5H), 2.15-2.04 (m, 1H), 2.00 (s, |
| | 3H), 1.94-1.83 (m, 1H), 1.83-1.59 (m, 2H). |
| I-182 | MS m/z 431.2 [M + H]⁺; ¹H NMR (METHANOL-d4) δ: 9.52 (s, 1H), 8.72 (d, |
| | J = 5.6 Hz, 1H), 8.06 (s, 1H), 7.83-7.99 (m, 1H), 7.65 (d, J = 8.0 Hz, 1H), 7.29 (d, |
| | J = 5.6 Hz, 1H), 4.51-4.68 (m, 1H), 3.59-3.77 (m, 1H), 3.28 (br s, 1H), 3.15-3.24 |
| | (m, 3H), 2.97 (br s, 1H), 2.71-2.76 (m, 1H), 2.07-2.15 (m, 1H), 2.04 (br s, 1H), |
| | 1.68-1.95 (m, 2H). |
| I-190 | MS m/z 418.2 [M + H]⁺; ¹H NMR (METHANOL-d4) δ: 9.77 (s, 1H), 8.91 (d, |
| | J = 5.6 Hz, 1H), 8.37 (s, 1H), 8.06 (s, 1H), 7.82 (d, J = 7.9 Hz, 1H), 7.60 (d, J = 7.9 |
| | Hz, 1H), 7.39 (dd, J = 5.8, 0.9 Hz, 1H), 4.69-4.84 (m, 1H), 4.53 (br s, 2H), 3.83 |
| | (br d, J = 9.0 Hz, 1H), 3.44 (br d, J = 11.5 Hz, 1H), 3.03-3.18 (m, 2H), 2.92 (s, |
| | 3H), 2.15-2.32 (m, 2H), 1.89-2.08 (m, 2H) |
| I-197 | MS m/z 421.4 [M + H]⁺; ¹H NMR (400 MHz, DMSO-d6) δ 9.82 (s, 1H), 8.85 (d, |
| | J = 5.6 Hz, 1H), 7.73 (d, J = 7.2 Hz, 1 H), 7.26 (d, J = 8.8 Hz, 1H), 7.23 (d, J = |
| | 5.6 Hz, 1H), 7.15 (s, 1H), 4.49-4.45 (m, 1H), 3.10 (d, J = 8.8 Hz, 1H), 2.74-2.71 |
| | (m, 1H), 2.23 (s, 3H), 2.04-1.90 (m, 3H), 1.79-1.76 (m, 1H), 1.66-1.57 (m, |
| | 1H), 1.52-1.45 (m, 1H) |
| I-220 | MS m/z 415 [M − H]⁻; ¹H NMR (400 MHz, DMSO-d6) δ 9.92 (br s, 1H), 8.41 |
| | (dd, J = 11.6, 8.4 Hz, 1H), 8.24 (s, 1H, formic acid), 7.90 (q, J = 7.2 Hz, 1H), |
| | 7.83 (t, J = 7.2 Hz, 1H), 7.70-7.59 (m, 2H), 7.49-7.45 (m, 2H), 7.31-7.20 |
| | (m, 2H), 4.61-4.59 (br s, 1H), 3.58-3.55 (m, 1H), 3.24-3.21 (m, 1H), |
| | 2.94-2.88 (m, 2H), 2.13-2.11 (m, 1H), 1.99-1.96 (m, 1H), 1.80-1.78 (m, 2H). |
| I-273 | MS m/z 398.5 [M + H]⁺; ¹H NMR (400 MHz, DMSO-d6) δ 9.88 (br s, 1 H), |
| | 9.78 (s, 1 H), 8.84 (d, J = 5.63 Hz, 1 H), 8.16 (s, 1 H, formic acid), 7.61 (d, J = |
| | 7.50 Hz, 1 H), 7.17 (d, J = 5.50 Hz, 1 H), 6.70-6.61 (m, 2 H), 4.57-4.34 (m, 1 |
| | H), 3.53 (t, J = 7.00 Hz, 2 H), 3.22-3.14 (m, 1 H), 2.84 (d, J = 11.26 Hz, 1 H), |
| | 2.49-2.44 (m, 3 H), 2.33 (s, 3 H), 2.18-1.96 (m, 3 H), 1.83-1.72 (m, 1 H), |
| | 1.69-1.43 (m, 2 H) |
| I-276 | MS m/z 498.4 [M + H]⁺; ¹H NMR (400 MHz, DMSO-d6) δ 9.78 (s, 1 H), 8.83 |
| | (d, J = 5.63 Hz, 1 H), 7.67 (d, J = 7.38 Hz, 1 H), 7.63-7.57 (m, 1 H), 7.52-7.46 |
| | (m, 2 H), 7.19 (d, J = 5.50 Hz, 1 H), 6.68 (t, J = 76.29 Hz, 1 H), 4.53-4.40 (m, |
| | 1 H), 3.94 (t, J = 5.75 Hz, 2 H), 3.77 (s, 3 H), 3.23-3.16 (m, 1 H), 2.90-2.82 |
| | (m, 1 H), 2.71-2.61 (m, 2 H), 2.18-1.98 (m, 3 H), 1.83-1.73 (m, 1 H), |
| | 1.67-1.44 (m, 2 H) |
| I-304 | MS m/z 368.5 [M + H]⁺; ¹H NMR (400 MHz, CD₃OD) δ 9.08 (d, J = 4.4 Hz, |
| | 1H), 7.89 (d, J = 8.3 Hz, 1H), 7.79 (dd, J = 8.4, 4.4 Hz, 1H), 6.67-6.58 (m, |
| | 2H), 4.56-4.47 (m, 1H), 3.13-2.96 (m, 1H), 2.70-2.52 (m, 1H), 2.50-2.24 |
| | (m, 7H), 2.11-1.96 (m, 1H), 1.94-1.82 (m, 1H), 1.80-1.60 (m, 2H). NH |
| | and OH not observed. 1H under solvent. |
| I-314 | MS m/z 359.4, 361.4 [M + H]⁺; ¹H NMR (400 MHz, CD₃OD) δ 7.54 (d, J = 8.4 |
| | Hz, 1H), 6.94 (s, 1H), 6.91 (d, J = 8.4 Hz, 1H), 4.46-4.28 (m, 1H), 3.26-3.12 |
| | (m, 3H), 2.95-2.76 (m, 3H), 2.61-2.29 (m, 5H), 2.20 (p, J = 7.5 Hz, 2H), |
| | 2.11-1.98 (m, 1H), 1.98-1.85 (m, 1H), 1.85-1.69 (m, 1H), 1.69-1.47 (m, |
| | 1H). 1 OH and 1 NH signals not observed. |

### Example 6

### Preparation of Compounds I-141 and I-142

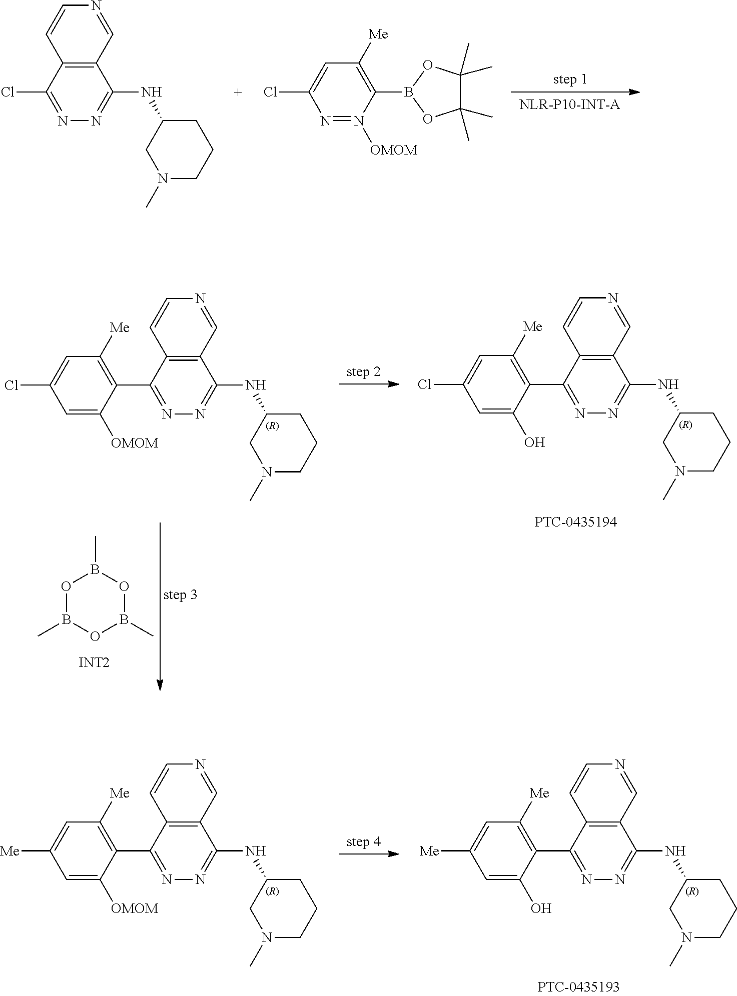

### Step 1. 1-(4-Chloro-2-(methoxymethoxy)-6-methylphenyl)-N—((R)-1-methylpiperidin-3-yl)pyrido[3,4-d]pyridazin-4-amine

To a solution of 1-chloro-N-[(3R)-1-methyl-3-piperidyl]pyrido[3,4-d]pyridazin-4-amine (Intermediate 20a, 150 mg, 0.54 mmol) in DMF (2 mL) and water (0.4 ml) was added K₂CO₃ (150 mg, 1.1 mmol), XPhos Pd G4 (49 mg, 0.05 mmol) and 2-[4-chloro-2-(methoxymethoxy)-6-methyl-phenyl]-4,4,5,5-tetramethyl-1,3,2-dioxaborolane (Intermediate 5 h, 169 mg, 0.54 mmol), heat to 100 degrees under the protection of nitrogen and stirred for 1 h, monitored by LCMS. After that, the mixture was concentrated and purified by silica gel column (DCM:MeOH=10:1) to give 1-(4-chloro-2-(methoxymethoxy)-6-methylphenyl)-N—((R)-1-methylpiperidin-3-yl)pyrido[3,4-d]pyridazin-4-amine as yellow oil. MS m/z 428.1 [M+H]⁺.

### Step 2. 5-Chloro-3-methyl-2-(4-(((R)-1-methylpiperidin-3-yl) amino)pyrido[3,4-d]pyridazin-1-yl)phenol

To a solution of 1-[4-chloro-2-(methoxymethoxy)-6-methyl-phenyl]-N-[(3R)-1-methyl-3-piperidyl]pyrido[3,4-d]pyridazin-4-amine (60 mg, 0.14 mmol) in DCM (2 mL) was added HCl in 1,4-dioxane (2 mL, 4 mol/L), stirred for 1 h, monitored by LCMS. After that, sodium bicarbonate saturated solution was added in to adjust pH to 8. The mixture was extracted with DCM (10 mL×3). The combined organic was dried over Na₂SO₄, concentrated to give a crude which was purified by Prep-HPLC to get 5-chloro-3-methyl-2-(4-(((R)-1-methylpiperidin-3-yl)amino)pyrido[3,4-d]pyridazin-1-yl)phenol (36 mg, 66.8% yield) as yellow solid. MS m/z 384.0 [M+H]⁺. ¹H NMR (400 MHz, DMSO-d6) δ 9.90 (s, 1H), 9.79 (s, 1H), 8.82 (d, J=5.6 Hz, 1H), 7.60 (dd, J=7.4, 1.7 Hz, 1H), 7.11-7.06 (m, 1H), 6.96-6.91 (m, 1H), 6.89-6.84 (m, 1H), 4.52-4.39 (m, 1H), 3.18-3.08 (m, 1H), 2.80-2.70 (m, 1H), 2.29-2.20 (m, 3H), 2.11-1.94 (m, 3H), 1.91 (d, J=6.7 Hz, 3H), 1.83-1.75 (m, 1H), 1.69-1.57 (m, 1H), 1.54-1.42 (m, 1H).

### Step 3. 1-(2-(Methoxymethoxy)-4,6-dimethylphenyl)-N—((R)-1-methylpiperidin-3-yl)pyrido[3,4-d]pyridazin-4-amine

To a solution of 1-[4-chloro-2-(methoxymethoxy)-6-methyl-phenyl]-N-[(3R)-1-methyl-3-piperidyl]pyrido[3,4-d]pyridazin-4-amine (100 mg, 0.23 mmol) in DMF (1 ml) and water (0.2 ml) was added K₂CO₃ (65 mg, 0.47 mmol), XPhos Pd G4 (22 mg, 0.023 mmol) and 2,4,6-trimethyl-1,3,5,2,4,6-trioxatriborinane (44 mg, 0.35 mmol), heat to 100 degrees under nitrogen and stir for 1 hour. After that, the mixture was concentrated to dry. Purification by SGC (DCM:MeOH=10:1) provided 1-(2-(methoxymethoxy)-4,6-dimethylphenyl)-N—((R)-1-methylpiperidin-3-yl)pyrido[3,4-d]pyridazin-4-amine (60 mg, 63% yield) as yellow oil. MS m/z 408.1 [M+H]⁺.

### Step 4. 3,5-Dimethyl-2-(4-(((R)-1-methylpiperidin-3-yl)amino)pyrido[3,4-d]pyridazin-1-yl)phenol

To a solution of 1-[2-(methoxymethoxy)-4,6-dimethyl-phenyl]-N-[(3R)-1-methyl-3-piperidyl]pyrido[3,4-d]pyridazin-4-amine (60 mg, 0.15 mmol) in DCM (2 mL) was added HCl in 1,4-dioxane (2 mL, 4 mol/L), and stirred for 1 h. After that, sodium bicarbonate saturated solution was added to adjust pH to 8. The mixture was extracted with DCM (10 mL×3). The combined organic was dried over Na₂SO₄, concentrated to give a crude which was purified by Prep-HPLC to get 3,5-dimethyl-2-(4-(((R)-1-methylpiperidin-3-yl)amino)pyrido[3,4-d]pyridazin-1-yl)phenol (36 mg, 67.2% Yield) as yellow solid. MS m/z 364.0 [M+H]⁺. ¹H NMR (400 MHz, DMSO-d6) δ 9.77 (d, J=1.1 Hz, 1H), 9.22 (d, J=8.7 Hz, 1H), 8.80 (d, J=5.6 Hz, 1H), 7.53 (dd, J=7.3, 2.0 Hz, 1H), 7.08 (dd, J=5.5 Hz, 1.1 Hz, 1H), 6.65 (s, 1H), 6.63 (d, J=2.0, 1H), 4.53-4.37 (m, 1H), 3.20-3.07 (m, 1H), 2.83-2.72 (m, 1H), 2.28 (s, 3H), 2.26 (d, J=5.3 Hz, 3H), 2.08-1.92 (m, 3H), 1.87 (d, J=6.9 Hz, 3H), 1.82-1.75 (m, 1H), 1.69-1.57 (m, 1H), 1.54-1.42 (m, 1H).

### Example 7

### Preparation of Compounds I-282, I-284, and I-285

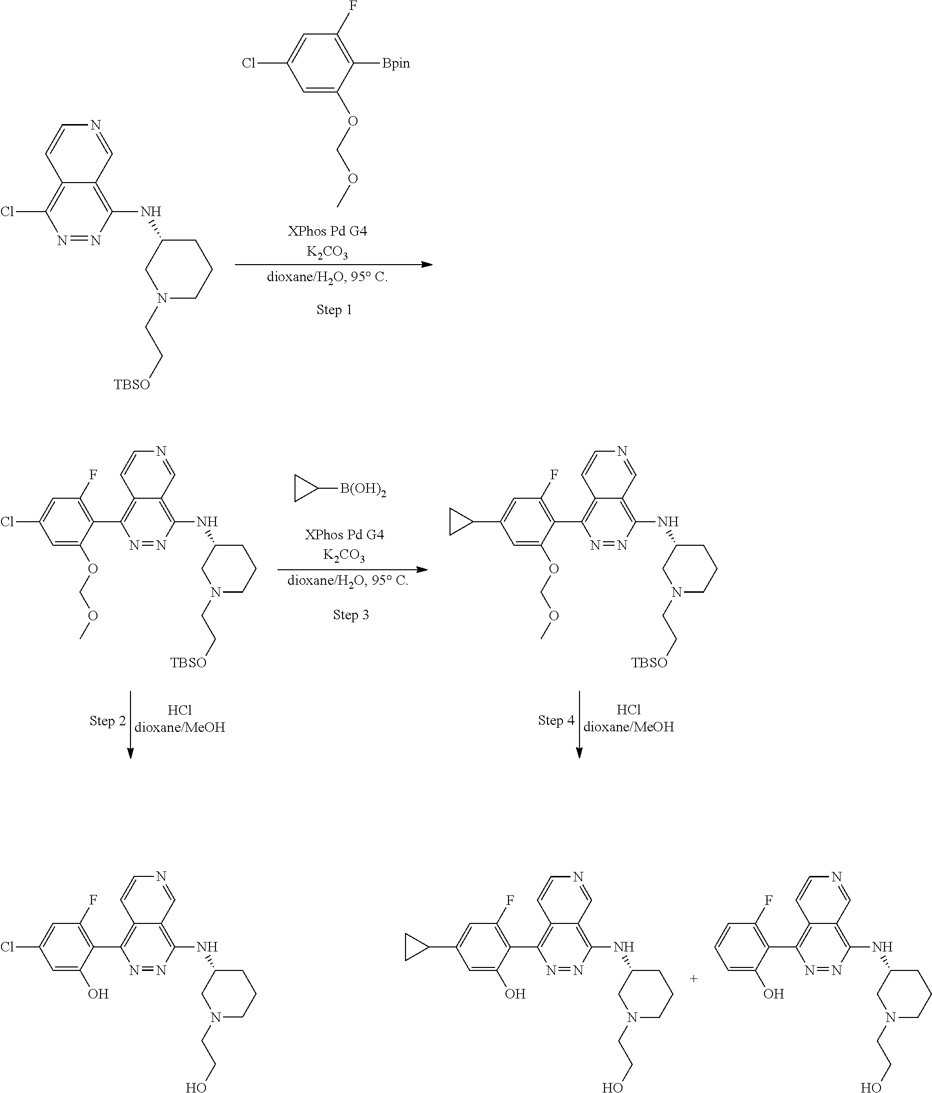

### Step 1: 1-[4-Chloro-2-fluoro-6-(methoxymethoxy)phenyl]-N-[(3R)-1-[2-[tert-butyl-(dimethyl)silyl]oxyethyl]-3-piperidyl]pyrido[3,4-d]pyridazin-4-amine

1-Chloro-N-[(3R)-1-[2-[tert-butyl(dimethyl)silyl]oxyethyl]-3-piperidyl]pyrido[3,4-d]pyridazin-4-amine (Intermediate 20c, 0.150 g, 0.355 mmol), 2-[4-chloro-2-fluoro-6-(methoxymethoxy)phenyl]-4,4,5,5-tetramethyl-1,3,2-dioxaborolane (Intermediate 5d, 0.197 g, 0.622 mmol), and XPhos Pd G4 (0.025 g, 0.028 mmol) were added to a vial and evacuated and refilled with Ar. The mixture was dissolved in dioxane (1.8 mL) and K₂CO₃ (2 M, 0.53 mL, 1.07 mmol). The mixture was sparged with Ar for 5 min. The reaction was then heated to 95° C. for 4 h. The mixture was diluted with EtOAc and filtered through Celite. The filtrate was washed with brine and concentrated. Purification by chromatography on SiO₂ (1M NH₄OH in MeOH:DCM, 0 to 10%) gave an orange foam. MS m/z 576.6 [M+H]⁺.

### Step 2. 5-Chloro-3-fluoro-2-[4-[[(3R)-1-(2-hydroxyethyl)-3-piperidyl]amino]pyrido[3,4-d]-pyridazin-1-yl]phenol formic acid salt

To 1-[2-fluoro-6-(methoxymethoxy)-4-methyl-phenyl]-N-[(3R)-1-[2-[tert-butyl(dimethyl)silyl]-oxyethyl]-3-piperidyl]pyrido[3,4-d]pyridazin-4-amine (0.070 g, 0.12 mmol) in MeOH (0.5 mL) was added HCl/dioxane (4M, 1.0 mL) and stirred for 1.5 h. The mixture was diluted with DCM/iPrOH (9:1) and washed with sat. NaHCO₃, brine, dried (Na₂SO₄), filtered and concentrated. Purification by reverse phase chromatography (0.1% formic acid in MeCN:0.1% formic acid in H₂O, 5 to 100%) gave a tan solid (0.013 g, 23%). MS m/z 418.4, 420.4 [M+H]⁺; ¹H NMR (400 MHz, DMSO-d₆) δ 9.79 (s, 1H), 8.84 (d, J=5.50 Hz, 1H), 8.17 (s, 1H, formic acid), 7.68 (d, J=7.63 Hz, 1H), 7.22 (d, J=5.63 Hz, 1H), 7.04 (d, J=9.13 Hz, 1H), 6.91 (s, 1H), 4.55-4.33 (m, 2H), 3.53 (t, J=6.00 Hz, 2H), 3.18 (d, J=10.3 Hz, 1H), 2.89-2.79 (m, 1H), 2.48-2.44 (m, 2H), 2.19-1.95 (m, 3H), 1.84-1.71 (m, 1H), 1.68-1.42 (m, 2H). 1H not observed (OH).

### Step 3: 1-[4-Cyclopropyl-2-fluoro-6-(methoxymethoxy)phenyl]-N-[(3R)-1-[2-[tert-butyl(dimethyl)silyl]oxyethyl]-3-piperidyl]pyrido[3,4-d]pyridazin-4-amine

1-[2-Fluoro-6-(methoxymethoxy)-4-methyl-phenyl]-N-[(3R)-1-[2-[tert-butyl(dimethyl)silyl]-oxyethyl]-3-piperidyl]pyrido[3,4-d]pyridazin-4-amine (0.140 g, 0.243 mmol), cyclopropyl boronic acid (0.042 g, 0.487 mmol), and XPhos Pd G4 (0.022 g, 0.024 mmol) were added to a vial and evacuated and refilled with Ar. The mixture was dissolved in dioxane (1.2 mL) and K₂CO₃ (2 M, 0.37 mL, 0.73 mmol). The mixture was sparged with Ar for 5 min. The reaction was then heated to 95° C. for 4 h. The mixture was diluted with EtOAc and filtered through Celite. The filtrate was washed with brine and concentrated. Purification by chromatography on SiO₂ (1M NH₄OH in MeOH:DCM, 0 to 10%) gave an tan foam, which is a mixture of the title compound and des-C₁ by-product. This mixture was applied to the next step without further purification. MS m/z 582.7 [M+H]⁺

### Step 4: 5-Cyclopropyl-3-fluoro-2-[4-[[(3R)-1-(2-hydroxyethyl)-3-piperidyl]amino]-pyrido[3,4-d]pyridazin-1-yl]phenol formic acid salt

HCl/dioxane (4M, 1.0 mL) was added to the intermediate prepared from step 3. The reaction was stirred for 1.5 h, then diluted with DCM/iPrOH (9:1) and washed with sat.

NaHCO₃, brine, dried (Na₂SO₄), filtered and concentrated. Purification by reverse phase chromatography (0.1% formic acid in MeCN:0.1% formic acid in H₂O, 5 to 100%) gave 5-cyclopropyl-3-fluoro-2-[4-[[(3R)-1-(2-hydroxyethyl)-3-piperidyl]amino]-pyrido[3,4-d]pyridazin-1-yl]phenol (27 mg, 24%) and 3-fluoro-2-[4-[[(3R)-1-(2-hydroxyethyl)-3-piperidyl]amino]pyrido[3,4-d]pyridazin-1-yl]phenol, respectively. 5-Cyclopropyl-3-fluoro-2-[4-[[(3R)-1-(2-hydroxyethyl)-3-piperidyl]amino]-pyrido[3,4-d]pyridazin-1-yl]phenol formic acid salt: MS m/z 424.5 [M+H]⁺; ¹H NMR (400 MHz, DMSO-d₆) δ 9.87 (br, 1H), 9.77 (s, 1H), 8.83 (d, J=5.63 Hz, 1H), 8.17 (s, 1H, formic acid), 7.60 (d, J=7.38 Hz, 1H), 7.18 (d, J=5.50 Hz, 1H), 6.58 (s, 1H), 6.52 (d, J=11.51 Hz, 1H), 4.56-4.38 (m, 2H), 3.53 (t, J=6.63 Hz, 2H), 3.21-3.14 (m, 1H), 2.87-2.79 (m, 1H), 2.48-2.42 (m, 2H), 2.16-1.88 (m, 4H), 1.81-1.70 (m, 1H), 1.68-1.45 (m, 2H), 1.05-0.95 (m, 2H), 0.77-0.69 (m, 2H).

3-Fluoro-2-[4-[[(3R)-1-(2-hydroxyethyl)-3-piperidyl]amino]pyrido[3,4-d]pyridazin-1-yl]phenol formic acid salt: MS m/z 384.5 [M+H]⁺; ¹H NMR (400 MHz, DMSO-d₆) δ 9.79 (s, 1H), 8.85 (d, J=5.38 Hz, 1H), 8.21 (s, 1H, formic acid), 7.63 (d, J=7.88 Hz, 1H), 7.42-7.31 (m, 1H), 7.16 (d, J=6.38 Hz, 1H), 6.89-6.77 (m, 2H), 4.54-4.32 (m, 2H), 3.53 (t, J=6.50 Hz, 2H), 3.21-3.13 (m, 1H), 2.87-2.78 (m, 1H), 2.48-2.42 (m, 2H), 2.15-1.94 (m, 3H), 1.83-1.71 (m, 1H), 1.66-1.43 (m, 2H). 1H not observed (OH).

### Example 8

### Preparation of Compounds I-81, I-90, and I-91

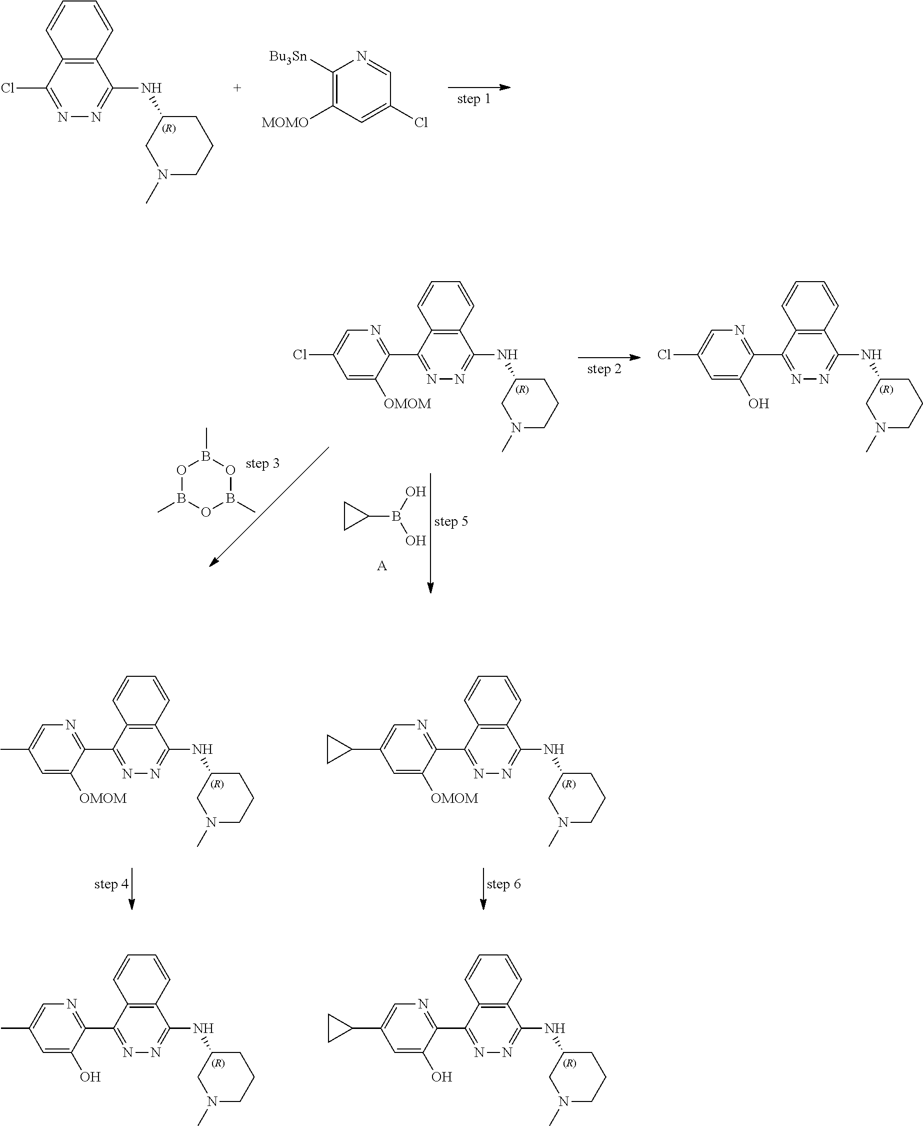

### Step 1. (R)-4-(5-Chloro-3-(methoxymethoxy)pyridin-2-yl)-N-(1-methylpiperidin-3-yl)phthalazin-1-amine

Argon was purged for 15 min through a stirring mixture of tributyl-[5-chloro-3-(methoxymethoxy)-2-pyridyl]stannane (prepared according to International patent application publication number WO2020231977, 1 g, 2.1 mmol), 4-chloro-N-[(3R)-1-methyl-3-piperidyl]phthalazin-1-amine (Intermediate 19a, 298 mg, 1 mmol) and cesium fluoride (650 mg, 4.27891 mmol) in DMF (15 mL). Cuprous iodide (40 mg, 0.2 mmol) and tetrakis(triphenylphosphine)palladium(0) (250 mg, 0.21 mmol) was added in and Ar was further purged for 10 min. The reaction mixture was allowed to stir at 100° C. for 1 h, monitored by LCMS. The residue was purified by column chromatography (8-10% MeOH in DCM) to afford product 4-[5-chloro-3-(methoxymethoxy)-2-pyridyl]-N-[(3R)-1-methyl-3-piperidyl]phthalazin-1-amine (180 mg, 0.43 mmol, 40% Yield). MS m/z 414.3 [M+H]⁺.

### Step 2. (R)-5-Chloro-2-(4-((1-methylpiperidin-3-yl)amino)phthalazin-1-yl)pyridin-3-ol

A solution of 4-[5-chloro-3-(methoxymethoxy)-2-pyridyl]-N-[(3R)-1-methyl-3-piperidyl]phthalazin-1-amine (120 mg, 0.28 mmol) in HCl (5 ml, 4M in 1,4-Dioxane) was stirred at rt. for 3 h, monitored by TLC and LCMS. After the reaction, solvent was removed and the residue was redissolved in DCM and H₂O. Aqueous solution of Na₂CO₃ was added to adjust the pH value to 8. This two-phase solution was extracted with DCM. The combined organic was dried over Na₂SO₄, and concentrated. The residue was purified by Prep-HPLC to give the product 5-chloro-2-[4-[[(3R)-1-methyl-3-piperidyl]amino]phthalazin-1-yl]pyridin-3-ol (45 mg, 42% Yield). MS m/z 370.0 [M+H]⁺; ¹H NMR (400 MHz, CDCl₃) δ 10.02-9.95 (m, 1H), 8.21 (d, J=2.0 Hz, 1H), 8.05-7.95 (m, 1H), 7.95-7.83 (m, 2H), 7.42 (d, J=2.0 Hz, 1H), 6.46 (s, 1H), 4.73-4.61 (m, 1H), 2.90-2.73 (m, 1H), 2.60-2.44 (m, 1H), 2.35 (s, 3H), 2.21-2.05 (m, 2H), 1.91-1.77 (m, 2H), 1.68-1.62 (m, 2H).

### Step 3. (R)-4-(3-(Methoxymethoxy)-5-methylpyridin-2-yl)-N-(1-methylpiperidin-3-yl)phthalazin-1-amine

To a solution of 4-[5-chloro-3-(methoxymethoxy)-2-pyridyl]-N-[(3R)-1-methyl-3-piperidyl]phthalazin-1-amine (600 mg, 1.4 mmol), was added 2,4,6-trimethyl-1,3,5,2,4,6-trioxatriborinane (108 g, 14 mmol), potassium carbonate (560 mg, 4 mmol) and XPhos Pd G4 (200 mg, 0.2 mmol) in DMF (10 ml) and water (1 mL). The mixture was stirred at 100° C. for 3 hours under N₂ atmosphere. After reaction, solvent was removed and the residue was purified by column chromatography (6%-8% MeOH in DCM) to afford product 4-[3-(methoxymethoxy)-5-methyl-2-pyridyl]-N-[(3R)-1-methyl-3-piperidyl]phthalazin-1-amine (180 mg, 32.2% Yield). MS m/z 394.0 [M+H]⁺.

### Step 4. (R)-5-Methyl-2-(4-((1-methylpiperidin-3-yl)amino)phthalazin-1-yl)pyridin-3-ol

A solution of 4-[3-(methoxymethoxy)-5-methyl-2-pyridyl]-N-[(3R)-1-methyl-3-piperidyl]phthalazin-1-amine (150 mg, 0.4 mmol) in HCl (5 mL, 4M in 1,4-dioxane) was stirred at rt. for 3 h, monitored by LCMS and TLC. After the reaction, solvent was removed. The residue was re-dissolved in H₂O and DCM, and NaHCO₃ was added in to adjust the pH value to 8. This mixture was extracted with DCM for 3 times. The combined organic was dried over Na₂SO₄ and concentrated to give the crude product which was purified by Prep-HPLC to afford 5-methyl-2-[4-[[(3R)-1-methyl-3-piperidyl]amino]phthalazin-1-yl]pyridin-3-ol (50 mg, 37.5% Yield). MS m/z 350.0[M+H]⁺; ¹H NMR (CHLOROFORM-d) δ: 9.98 (d, J=8.0 Hz, 1H), 8.11 (s, 1H), 7.97 (br d, J=7.5 Hz, 1H), 7.79-7.92 (m, 2H), 7.22-7.24 (m, 1H), 6.33 (br s, 1H), 4.67 (br s, 1H), 2.78 (br s, 2H), 2.45-2.57 (m, 1H), 2.37 (s, 3H), 2.33 (s, 3H), 2.14 (br d, J=10.5 Hz, 1H), 1.98-2.08 (m, 1H), 1.95 (br s, 1H), 1.77-1.88 (m, 2H), 1.62 (br d, J=3.5 Hz, 1H)

### Step 5. (R)-4-(5-Cyclopropyl-3-(methoxymethoxy)pyridin-2-yl)-N-(1-methylpiperidin-3-yl)phthalazin-1-amine

To a solution of cyclopropylboronic acid (500 mg, 5.8 mmol) in DMF (3 mL, 38.8 mmol) was added 4-[5-chloro-3-(methoxymethoxy)-2-pyridyl]-N-[(3R)-1-methyl-3-piperidyl]phthalazin-1-amine (300 mg, 0.7 mmol), sodium carbonate (200 mg, 1.8 mmol) and XPhos Pd G4 (30 mg, 0.03 mmol). The mixture was stirred at 100° C. for 1 h under N₂ atmosphere. After the reaction, the mixture was poured into H₂O, extracted with EA(2×30 mL). The organic layers were dried over Na₂SO₄, concentrated and purify via flash chromatography (CH₂Cl₂/MeOH=92:8) to give 4-[5-cyclopropyl-3-(methoxymethoxy)-2-pyridyl]-N-[(3R)-1-methyl-3-piperidyl]phthalazin-1-amine (80 mg, 26.3% Yield) as a solid. MS m/z 420.0 [M+H]⁺.

### Step 6. (R)-5-Cyclopropyl-2-(4-((1-methylpiperidin-3-yl)amino)phthalazin-1-yl)pyridin-3-ol

A solution of 4-[5-cyclopropyl-3-(methoxymethoxy)-2-pyridyl]-N-[(3R)-1-methyl-3-piperidyl]phthalazin-1-amine (80 mg, 0.2 mmol) in HCl (5 mL, 4M in 1,4-dioxane) was stirred at 25° C. for 0.5 h, monitored by LCMS. After the reaction, solvent was removed. The residue was re-solved in H₂O and DCM, and NaHCO₃ was added in to adjust the pH value to 8. This mixture was extracted with DCM for 3 times. The combined organic was dried over Na₂SO₄ and concentrated to give the crude product which was purified by Prep-HPLC to afford 5-cyclopropyl-2-[4-[[(3R)-1-methyl-3-piperidyl]amino]phthalazin-1-yl]pyridin-3-ol (45 mg, 62.8% Yield) as a solid. MS m/z 376.2 [M+H]⁺; ¹H NMR (400 MHz, DMSO-d6) δ 14.65 (s, 1H), 9.80-9.72 (m, 1H), 8.52-8.42 (m, 1H), 8.13 (d, J=2.0 Hz, 1H), 8.00-7.91 (m, 2H), 7.49 (d, J=7.6 Hz, 1H), 7.02 (d, J=2.0 Hz, 1H), 4.48-4.38 (m, 1H), 3.13-3.05 (m, 1H), 2.78-2.71 (m, 1H), 2.25 (s, 3H), 2.05-1.91 (m, 4H), 1.81-1.70 (m, 1H), 1.68-1.56 (m, 1H), 1.55-1.44 (m, 1H), 1.08-1.01 (m, 2H), 0.86-0.77 (m, 2H).

### Example 9

### Preparation of Compound I-226

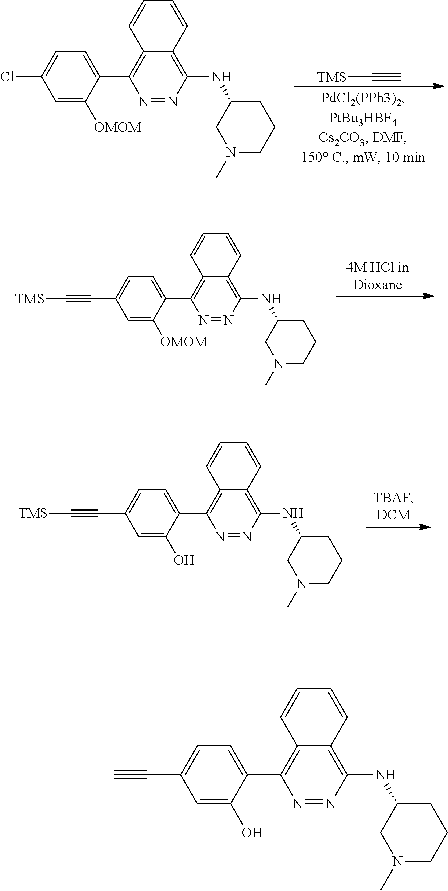

### Step 1. (R)-4-(2-(Methoxymethoxy)-4-((trimethylsilyl)ethynyl)phenyl)-N-(1-methylpiperidin-3-yl)phthalazin-1-amine

A mixture of (R)-5-chloro-2-(4-((1-methylpiperidin-3-yl)amino)phthalazin-1-yl)phenol (prepared according to the procedure of Example 1, 41.4 mg, 0.1 mmol, 1.0 eq.), ethynyl(trimethyl)silane (86 μL, 0.6 mmol, 6.0 eq), Cs₂CO₃ (33 mg, 0.1 mmol, 1.0 eq.), PdCl₂(PPh3)₂ (2.1 mg, 0.003 mmol, 0.03 eq.), tri-tert-butylphosphonium tetrafluoroborate (1.7 mg, 0.006 mmol, 0.06 eq.), and DBU (1.5 μL, 0.01 mmol, 0.1 eq) in DMF (0.7 mL) was sparged with argon and then heated to 150° C. via microwave irradiation for 10 min. The reaction was then cooled to ambient temperature. The crude reaction mixture was diluted with ethyl acetate and filtered through celite. The filtrate was washed with brine, and the combined aqueous phase was extracted with ethyl acetate. The combined organic layers were dried over Na₂SO₄ and concentrated in vacuo. The crude residue was purified by silica gel column chromatography eluting with 0:100 to 10:90 (10% NH₄OH in MeOH):DCM to afford (R)-4-(2-(methoxymethoxy)-4-((trimethylsilyl)ethynyl)phenyl)-N-(1-methylpiperidin-3-yl)phthalazin-1-amine as an impure oil. The mixture was carried forward without further purification. MS m/z 475.7 [M+H]⁺.

### Step 2. (R)-2-(4-((1-Methylpiperidin-3-yl)amino)phthalazin-1-yl)-5-((trimethylsilyl)ethynyl) phenol

The mixture from step 1 was suspended in 4 M HCl in dioxane (2 mL) and stirred at room temperature for 4 h. Upon reaction completion solvents were removed in vacuo. The residue was concentrated several times from dichloromethane to remove excess HCl. The mixture was carried forward without further purification. MS m/z 431.6 [M+H]⁺.

### Step 3. (R)-5-Ethynyl-2-(4-((1-methylpiperidin-3-yl)amino)phthalazin-1-yl)phenol; formic acid salt

The crude mixture from step 2 was dissolved in DCM (2 mL) and TBAF (110 μL, 2 eq, 1 M in THF) was added. The reaction was stirred at rt for 3 h. Upon completion, the crude reaction mixture was diluted with sodium bicarbonate (saturated, aq.) and extracted several times with EtOAc. The combined organic layers were dried over Na₂SO₄ and concentrated in vacuo. Purification by C18 reverse phase Prep-HPLC eluting with ACN:Water with formic acid as the modifier afforded (R)-5-ethynyl-2-(4-((1-methylpiperidin-3-yl)amino)phthalazin-1-yl)phenol formic acid salt (8.2 mg, 20% over 3 steps). MS m/z 359.5 [M+H]⁺. ¹H NMR (400 MHz, CD₃OD) δ 8.54 (s, 1H), 8.29 (d, J=8.2 Hz, 1H), 7.89 (t, J=7.6 Hz, 1H), 7.84-7.78 (m, 1H), 7.65 (d, J=8.1 Hz, 1H), 7.32 (d, J=7.8 Hz, 1H), 7.12 (dd, J=7.7, 1.5 Hz, 1H), 7.08 (d, J=1.4 Hz, 1H), 4.68-4.54 (m, 1H), 3.55 (s, 1H), 3.45-3.36 (m, 1H), 3.04-2.91 (m, 1H), 2.74-2.48 (m, 5H), 2.21-2.06 (m, 1H), 2.05-1.93 (m, 1H), 1.91-1.61 (m, 2H).

### Example 10

### Preparation of Compounds I-143 and I-194

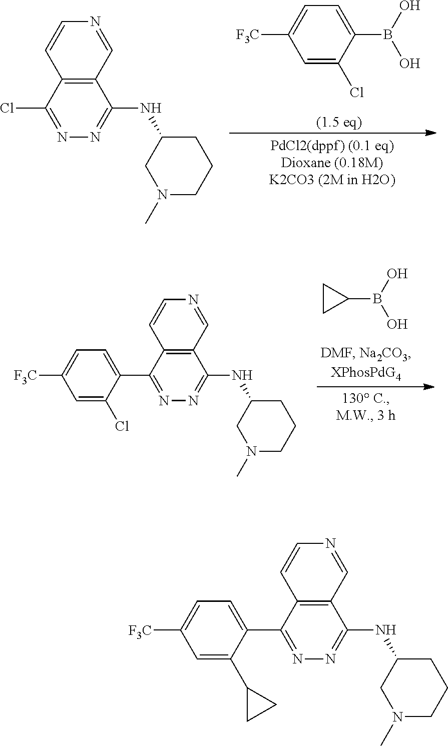 [SMILES: CN1CCC[C@@H](Nc2nnc(Cl)c3ccncc23)C1 ; OB(O)c1ccc(C(F)(F)F)cc1Cl ; CN1CCC[C@@H](Nc2nnc(-c3ccc(C(F)(F)F)cc3Cl)c3ccncc23)C1 ; OB(O)C1CC1 ; CN1CCC[C@@H](Nc2nnc(-c3ccc(C(F)(F)F)cc3C3CC3)c3ccncc23)C1]

### Step 1. (R)-1-(2-Chloro-4-(trifluoromethyl)phenyl)-N-(1-methylpiperidin-3-yl)pyrido[3,4-d]pyridazin-4-amine

The title compound was prepared according to the procedure of Example 1. MS m/z 422.2, 424.2 [M+H]⁺; ¹H NMR (400 MHz, DMSO-d6) δ 9.85 (s, 1H), 8.86 (d, J=5.6 Hz, 1H), 8.11 (s, 1H), 7.93 (d, J=8.0 Hz, 1H), 7.81 (d, J=7.6 Hz, 1H), 7.20 (d, J=5.6 Hz, 1H), 4.50-4.48 (m, 1H), 3.10 (d, J=8.8 Hz, 1H), 2.73 (d, J=10.8 Hz, 4H), 2.04-1.91 (m, 3H), 1.79-1.73 (m, 1H), 1.66-1.56 (m, 1H), 1.52-1.43 (m, 1H).

### Step 2. (R)-1-(2-Cyclopropyl-4-(trifluoromethyl)phenyl)-N-(1-methylpiperidin-3-yl)pyrido[3,4-d]pyridazin-4-amine

To a mixture of 1-[2-chloro-4-(trifluoromethyl)phenyl]-N-[(3R)-1-methyl-3-piperidyl]pyrido[3,4-d]pyridazin-4-amine (100 mg, 0.237 mmol) in DMF (2 mL) were added Na₂CO₃ (64 mg, 0.604 mmol), XPhosPdG₄ (8 mg, 0.009 mmol), cyclopropylboronic acid (163 mg, 1.90 mmol). The mixture was stirred for 3 h at 130° C. under microwave. After completion, the reaction mixture was cooled to room temperature and filtered. The filtrate was diluted with water and extracted with ethyl acetate (20 mL×3). The combined organic layer was washed with brine, dried over anhydrous Na₂SO₄ and concentrated under reduced pressure. The crude product was purified by prep-HPLC to afford title product (95 mg, 93.8% Yield) as a white solid. MS m/z 428.2 [M+H]⁺, ¹H NMR (400 MHz, CD₃OD) δ 9.80 (s, 1H), 8.96 (d, J=5.6 Hz, 1H), 7.69 (d, J=7.9 Hz, 1H), 7.55 (d, J=7.9 Hz, 1H), 7.47-7.36 (m, 2H), 4.78-4.67 (m, 1H), 4.10-3.95 (m, 1H), 3.71-3.54 (m, 1H), 3.10-2.88 (m, 5H), 2.44-2.28 (m, 1H), 2.27-2.14 (m, 1H), 2.10-1.81 (m, 2H), 1.69-1.57 (m, 1H), 0.90-0.78 (m, 2H), 0.78-0.60 (m, 2H).

### Example 11

### Preparation of Compound I-228

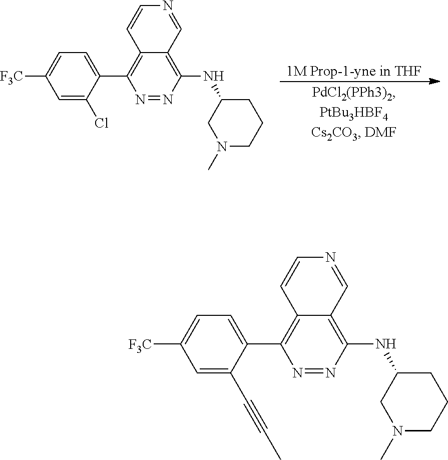 [SMILES: CN1CCC[C@@H](Nc2nnc(-c3ccc(C(F)(F)F)cc3Cl)c3ccncc23)C1 ; CC#Cc1cc(C(F)(F)F)ccc1-c1nnc(N[C@@H]2CCCN(C)C2)c2cnccc12]

A mixture of (R)-1-(2-chloro-4-(trifluoromethyl)phenyl)-N-(1-methylpiperidin-3-yl)pyrido[3,4-d]pyridazin-4-amine (Example 10, step 1, 163 mg, 0.15 mmol, 1 eq), prop-1-yne (1M in THF, 900 μL, 0.9 mmol, 6 eq), Cs₂CO₃ (33 mg, 0.1 mmol, 1 eq), PdCl₂(PPh3)₂ (3.2 mg, 0.0045 mmol, 0.03 eq), tri-tert-butylphosphonium tetrafluoroborate (2.6 mg, 0.009 mmol, 0.06 eq), and DBU (2.2 μL, 0.015 mmol, 0.1 eq) in DMF (1.1 mL) was sparged with argon and then heated to 110° C. via microwave irradiation for 3 h. After the reaction was cooled to ambient temperature, the crude reaction mixture was diluted with ethyl acetate and then filtered through celite. The filtrate was washed with brine, and the combined aqueous phase was extracted with ethyl acetate. The combined organic layers were dried over Na₂SO₄ and concentrated in vacuo.

The crude residue was purified by silica gel column chromatography eluting with 0:100 to 10:90 (10% NH₄OH in MeOH):DCM followed by C18 reverse phase Prep-HPLC eluting with ACN:Water with formic acid as the modifier to afford (R)—N-(1-methylpiperidin-3-yl)-1-(2-(prop-1-yn-1-yl)-4-(trifluoromethyl)phenyl)pyrido[3,4-d]pyridazin-4-amine formic acid salt (2.2 mg, 3.13). MS m/z 426.5 [M+H]⁺; ¹H NMR (400 MHz, C₃Od) δ 9.74 (s, 1H), 8.86 (d, J=5.7 Hz, 1H), 8.54 (s, 1H), 7.87 (s, 1H), 7.81 (d, J=8.1 Hz, 1H), 7.69 (d, J=8.1 Hz, 1H), 7.40 (d, J=5.8 Hz, 1H), 4.73-4.60 (mi, 1H), 3.30-3.25 (i, 1H), 2.95-2.75 (m, 1H), 2.58-2.35 (m, 5H), 2.20-2.05 (m, 1H), 2.03-1.89 (7, 1H), 1.88-1.68 (m, 2H), 1.65 (s, 3H).

The compounds below were prepared according to the procedure of Example 11 by substituting the appropriate starting materials, reagents and reaction conditions.

| Compound | Spectral Data |
|---|---|
| I-13 | MS m/z 369.1 [M + H]⁺; ¹H NMR (DMSO-d6) δ: 10.16 (s, 1H), 8.38 (d, J = 8.1 |
| | Hz, 1H), 7.84 (dd, J = 11.2, 4.1 Hz, 1H), 7.78 (t, J = 7.2 Hz, 1H), 7.46 (d, J = |
| | 7.7 Hz, 1H), 7.33-7.27 (m, 1H), 7.10 (d, J = 7.3 Hz, 1H), 7.02 (s, 1H), 7.02- |
| | 7.00 (m, 1H), 4.48-3.36 (s, 1H), 3.15-3.05 (m, 1H), 2.78-2.71 (m, 1H), |
| | 2.26 (s, 3H), 2.10-1.87 (m, 3H), 1.82-1.73 (s, 1H), 1.70-1.55 (m, 1H), 1.54- |
| | 1.40 (m, 1H). |
| I-32 | MS m/z 419.3 [M + H]⁺; ¹H NMR (MeOH-d4) δ: 8.32 (d, J = 8 Hz, 1H), 7.85- |
| | 7.97 (m, 3H), 7.82 (t, J = 7 Hz, 1H), 7.73 (d, J = 8 Hz, 1H), 7.50 (d, J = 8 Hz, |
| | 1H), 4.40-4.46 (m, 1H), 3.40-3.45 (m, 1H), 3.00-3.05 (m, 1H), 2.91 (s, 3H), |
| | 2.71 (t, J = 10 Hz, 2H), 2.20-2.25 (m, 1H), 1.88-1.95 (m, 1H), 1.65-1.83 (m, |
| | 2H). Three exchangeable protons are not seen. |
| I-37 | MS m/z 402.7 [M + H]⁺; ¹H NMR (MeOH-d4) δ: 8.61 (s, 1H), 8.33 (d, J = 8 Hz, |
| | 1H), 7.95 (s, 1H), 7.91 (t, J = 8 Hz, 1H), 7.84 (t, J = 8 Hz, 1H), 7.73 (d, J = 8 |
| | Hz, 1H), 7.56-7.60 (m, 2H), 7.50 (d, J = 8 Hz, 1H), 4.59-4.64 (m, 1H), 3.05- |
| | 3.12 (m, 1H), 2.70-2.80 (m, 1H), 2.30-2.50 (m, 5H), 2.05-2.15 (m, 1H), 1.88- |
| | 1.97 (m, 1H), 1.50-1.85 (m, 2H). Two exchangeable protons are not seen. |
| I-38 | MS m/z 433.3 [M + H]⁺; ¹H NMR (MeOH-d4) δ: 8.32 (d, J = 8 Hz, 1H), 7.85- |
| | 7.97 (m, 3H), 7.82 (t, J = 7 Hz, 1H), 7.73 (d, J = 8 Hz, 1H), 7.50 (d, J = 8 Hz, |
| | 1H), 4.55-4.65 (m, 1H), 3.05-3.10 (m, 1H), 2.91 (s, 3H), 2.68-2.76 (m, 1H), |
| | 2.30-2.45 (m, 5H), 2.03-2.17 (m, 1H), 1.60-1.95 (m, 3H). Two exchangeable |
| | protons are not seen. |
| I-39 | MS m/z 411.1 [M + H]⁺; ¹H NMR (400 MHz, MeOD-d4) δ 8.28 (d, J = 8.2 Hz, |
| | 1H), 7.88-7.82 (m, 1H), 7.80-7.72 (m, 2H), 7.68-7.63 (m, 2H), 7.47-7.39 |
| | (m, 3H), 7.38-7.32 (m, 1H), 7.28-7.22 (m, 2H), 4.62-4.53 (m, 1H), 3.21- |
| | 3.03 (m, 1H), 2.75-2.61 (m, 1H), 2.36 (s, 5H), 2.10-1.97 (m, 1H), 1.92- |
| | 1.82 (m, 1H), 1.80-1.57 (m, 2H). |
| I-40 | MS m/z 411.0 [M + H]⁺; ¹H NMR (400 MHz, DMSO-d6) δ 9.82 (s, 1H), 8.39 (d, |
| | J = 8.0 Hz, 1H), 7.88-7.82 (m, 1H), 7.82-7.75 (m, 1H), 7.68-7.61 (m, 3H), |
| | 7.59-7.53 (m, 2H), 7.42 (t, J = 7.6 Hz, 2H), 7.29 (t, J = 7.2 Hz, 1H), 7.11- |
| | 7.05 (m, 2H), 4.51-4.38 (m, 1H), 3.14-3.06 (m, 1H), 2.76-2.69 (m, 1H), |
| | 2.23 (s, 3H), 2.01-1.88 (m, 3H), 1.80-1.70 (m, 1H), 1.69-1.55 (m, 1H), |
| | 1.54-1.39 (m, 1H). |
| I-49 | MS m/z 375.0 [M + H]⁺; ¹H NMR (400 MHz, CDCl₃) δ: 8.27 (d, J = 8.0 Hz, 1H), |
| | 8.10 (d, J = 7.6 Hz, 1H), 7.91-7.74 (m, 2H), 7.47 (d, J = 8.0 Hz, 1H), 6.85 (d, |
| | J = 15.6 Hz, 1H), 6.76-6.71 (m, 1H), 4.77-4.65 (m, 1H), 2.88-2.85 (m, 2H), |
| | 2.56-2.54 (m, 1H), 2.36 (s, 3H), 2.20-2.09 (m, 3H), 1.96-1.84 (m, 2H), 1.67- |
| | 1.64 (m, 2H), 1.34-1.20 (m, 1H), 1.07-0.95 (m, 2H), 0.87-0.74 (m, 2H). |
| I-52 | MS m/z 417.3 [M + H]⁺; ¹H NMR (400 MHz, DMSO-d6) δ: 9.78 (s, 1H), 8.38 |
| | (d, J = 8.0 Hz, 1H), 7.92-7.74 (m, 2H), 7.53 (d, J = 8.0 Hz, 1H), 7.26 (d, J = |
| | 8.0 Hz, 1H), 7.14 (s, 1H), 7.01-6.91 (m, 2H), 6.33 (s, 1H), 4.45 (s, 3H), 3.81- |
| | 3.72 (m, 2H), 2.88 (s, 1H), 2.33 (d, J = 32.0 Hz, 5H), 2.23-2.10 (m, 2H), 2.06- |
| | 1.95 (m, 2H), 1.88-1.77 (m, 1H), 1.72-1.60 (m, 1H), 1.59-1.48 (m, 1H). |
| I-58 | MS m/z 416.0 [M + H]⁺; ¹H NMR (400 MHz, MeOD-d4-d4) δ: 8.29 (d, J = 8.0 |
| | Hz, 1H), 7.89-7.84 (m, 1H), 7.82-7.76 (m, 1H), 7.68 (d, J = 8.0 Hz, 1H), |
| | 7.34 (d, J = 8.0 Hz, 1H), 7.12-7.06 (m, 1H), 7.02 (d, J = 4.0 Hz, 1H), 6.28 (s, |
| | 1H), 4.63-4.53 (m, 1H), 3.96-3.89 (m, 2H), 3.79-3.71 (m, 2H), 3.25-3.15 |
| | (m, 1H), 2.80-2.70 (m, 1H), 2.62 (s, 3H), 2.45-2.33 (m, 5H), 2.00-2.10 (s, |
| | 1H), 1.95-1.85 (m, 1H), 1.83-1.60 (m, 2H). |
| I-59 | MS m/z 370.1 [M + H]⁺; ¹H NMR (400 MHz, CD₃OD) δ 9.69 (s, 1H), 8.85 (d, J = |
| | 4.0 Hz, 1H), 8.55 (s, 1H), 7.50 (d, J = 4.0 Hz, 1H), 7.34 (d, J = 7.6 Hz, 1H), |
| | 7.04-7.03 (m, 2H), 4.71 (m, 1H), 3.64 (m, 1H), 3.23 (m, 1H), 2.92 (m, 2H), |
| | 2.76 (s, 3H), 2.15 (m, 2H), 1.91 (m, 2H). |
| I-60 | MS m/z 370.1 [M + H]⁺; ¹H NMR (400 MHz, CD₃OD) δ 8.95 (m, 2H), 8.54 (s, |
| | 1H), 8.22 (s, 1H), 7.40 (d, J = 8.0 Hz, 1H), 7.08-7.06 (m, 2H), 4.65 (m, 1H), |
| | 3.61 (m, 1H), 3.21 (m, 1H), 2.87 (m, 2H), 2.74 (s, 3H), 2.13 (m, 2H), 1.98- |
| | 1.69 (m, 2H). |
| I-70 | MS m/z 387.0 [M + H]⁺; ¹H NMR (400 MHz, DMSO-d6) δ 10.47 (s, 1H), 8.41 |
| | (d, J = 8.3 Hz, 1H), 7.86 (t, J = 11.2, 4.1 Hz, 1H), 7.79 (t, J = 7.1 Hz, 1H), 7.36 |
| | (d, J = 8.1 Hz, 1H), 7.17 (d, J = 7.7 Hz, 1H), 7.02 (dd, J = 9.0, 1.9 Hz, 1H), |
| | 6.94-6.85 (m, 1H), 4.51-4.36 (m, 1H), 3.17-3.04 (m, 1H), 2.83-2.69 (m, |
| | 1H), 2.23 (s, 3H), 2.03-1.85 (m, 3H), 1.81-1.71 (m, 1H), 1.66-1.56 (m, |
| | 1H), 1.54-1.41 (m, 1H). |
| I-76 | MS m/z 370.0 [M + H]⁺; ¹H NMR (400 MHz, MeOD-d4) δ 9.63 (d, J = 0.8 Hz, |
| | 1H), 8.79 (d, J = 5.6 Hz, 1H), 7.52-7.45 (m, 2H), 7.31 (dd, J = 7.6, 1.6 Hz, |
| | 1H), 6.97 (t, J = 7.6 Hz, 1H), 4.66-4.54 (m, 1H), 3.24-3.04 (m, 1H), 2.85- |
| | 2.64 (m, 1H), 2.52-2.26 (m, 5H), 2.12-1.97 (m, 1H), 1.96-1.83 (m, 1H), |
| | 1.82-1.55 (m, 2H). |
| I-77 | MS m/z 376.0 [M + H]⁺; ¹H NMR (400 MHz, MeOD-d4) δ 9.71 (s, 1H), 8.87 (d, |
| | J = 5.6 Hz, 1H), 7.61 (dd, J = 5.6, 0.8 Hz, 1H), 7.23 (dd, J = 7.6, 1.6 Hz, 1H), |
| | 7.09 (d, J = 7.6 Hz, 1H), 7.02-6.96 (m, 1H), 4.70-4.58 (m, 1H), 3.31-3.19 |
| | (m, 1H), 2.94-2.81 (m, 1H), 2.58-2.39 (m, 5H), 2.19-2.07 (m, 2H), 2.00- |
| | 1.90 (m, 1H), 1.88-1.65 (m, 3H), 1.03-0.93 (m, 2H), 0.74-0.68 (m, 2H). |
| I-89 | MS m/z 376.0 [M + H]⁺. ¹H NMR (400 MHz, DMSO-d6) δ 9.50 (s, 1H), 8.94 (d, |
| | J = 5.7 Hz, 1H), 8.03 (d, J = 5.7 Hz, 1H), 7.45 (d, J = 8.0 Hz, 1H), 6.84 (d, J = |
| | 1.9 Hz, 1H), 6.75 (dd, J = 8.0, 1.9 Hz, 1H), 4.79-4.64 (m, 1H), 2.92-2.74 (m, |
| | 2H), 2.58-2.45 (m, 1H), 2.35 (s, 3H), 2.18-2.05 (m, 2H), 1.97-1.78 (m, |
| | 2H), 1.73-1.56 (m, 2H), 1.07-0.98 (m, 2H), 0.83-0.74 (m, 2H). |
| I-93 | MS m/z 404.2 [M + H]⁺; ¹H NMR (400 MHz, MeOD-d4) δ 8.25 (d, J = 8.0 Hz, |
| | 1H), 7.96-7.73 (m, 3H), 7.22 (d, J = 8.4 Hz, 1H), 6.27 (dd, J = 8.4, 2.0 Hz, 1H), |
| | 6.19 (d, J = 2.0 Hz, 1H), 4.57-4.46 (m, 1H), 3.37-3.31 (m, 4H), 3.18-3.02 |
| | (m, 1H), 2.75-2.60 (m, 1H), 2.36 (s, 5H), 2.14-1.94 (m, 5H), 1.91-1.82 (m, |
| | 1H), 1.81-1.71 (m, 1H), 1.69-1.55 (m, 1H). |
| I-107 | MS m/z 418.3 [M + H]⁺; ¹H NMR (400 MHz, DMSO-d6) δ: 8.37 (d, J = 8.0 Hz, |
| | 1H), 7.88-7.81 (m, 1H), 7.81-7.74 (m, 1H), 7.51 (d, J = 4.0 Hz, 1H), 7.22 (d, |
| | J = 8.0 Hz, 1H), 7.09 (s, 1H), 6.93 (s, 1H), 6.89 (d, J = 8.0 Hz, 1H), 4.50- |
| | 4.404 (m, 1H), 3.45-3.34 (m, 1H), 3.18-3.07 (m, 2H), 2.94-2.83 (m, 2H), |
| | 2.83-2.76 (m, 1H), 2.77-2.69 (m, 1H), 2.48 (s, 3H), 2.29 (s, 3H), 2.14-1.93 |
| | (m, 4H), 1.93-1.86 (m, 1H), 1.83-1.74 (m, 1H), 1.70-1.58 (m, 1H), 1.55- |
| | 1.44 (m, 1H). |
| I-130 | MS m/z 412.8 [M + H]⁺, ¹H NMR (METHANOL-d4) δ: 9.78-9.82 (m, 1H), 8.92 |
| | (d, J = 5.6 Hz, 1H), 8.38 (s, 1H), 8.20 (d, J = 8.5 Hz, 1H), 7.98 (d, J = 8.1 Hz, 1H), |
| | 7.49-7.54 (m, 1H), 4.69 (dt, J = 8.5, 4.5 Hz, 1H), 3.08-3.25 (m, 1H), 2.75 (br s, |
| | 1H), 2.32-2.49 (m, 5H), 2.11 (br s, 1H), 1.87-1.97 (m, 1H), 1.68-1.86 (m, 2H). |
| I-132 | MS m/z 420.3 [M + H]⁺; ¹H NMR (400 MHz, DMSO-d6) δ 9.82 (s, 1H), 8.86 (d, |
| | J = 5.6 Hz, 1H), 7.72 (d, J = 7.6 Hz, 1H), 7.60-7.57 (m, 1H), 7.53 (s, 1H), 7.53- |
| | 7.52 (m, 1H), 7.26 (s, 1H), 7.25 (t, J = 81.2 Hz, 1H), 4.47-4.43 (m, 1H), 3.09 (d, |
| | J = 8.0 Hz, 1H), 2.72 (d, J = 10.8 Hz, 1H), 2.22 (s, 3H), 2.03-1.89 (m, 3H), 1.79- |
| | 1.75 (m, 1H), 1.66-1.57 (m, 1H), 1.51-1.42 (m, 1H). |
| I-133 | MS m/z 419.1, 421.1 [M + H]⁺; ¹H NMR (500 MHz, METHANOL-d4) δ 8.40 (s, |
| | 1H, formic acid), 8.35 (d, J = 9.0 Hz, 1H), 7.95 (t, J = 7.7 Hz, 1H), 7.86 (t, J = |
| | 7.6 Hz, 1H), 7.56-7.46 (m, 4H), 6.83 (td, J = 73.5, 13.4 Hz, 1H), 4.72-4.63 |
| | (m, 1H), 3.91-3.66 (m, 1H), 3.51-3.38 (m, 1H), 3.13-2.96 (m, 2H), 2.89 (s, |
| | 3H), 2.34-2.12 (m, 2H), 2.03-1.81 (m, 2H); 1H not observed (NH) |
| I-134 | MS m/z 400.2 [M + H]⁺; ¹H NMR (500 MHz, METHANOL-d4) δ 9.72 (s, 1H), |
| | 8.89 (d, J = 5.6 Hz, 1H), 8.36 (s, 1H, formic acid), 7.42 (d, J = 7.0 Hz, 2H), |
| | 7.29 (d, J = 7.8 Hz, 1H), 7.24 (s, 1H), 6.78 (td, J = 74.2, 8.2 Hz, 1H), 4.76- |
| | 4.69 (m, 1H), 3.92-3.77 (m, 1H), 3.50-3.37 (m, 1H), 3.15-2.96 (m, 2H), |
| | 2.90 (s, 3H), 2.51 (s, 3H), 2.33-2.14 (m, 2H), 2.03-1.84 (m, 2H); 1H not |
| | observed (NH) |
| I-135 | MS m/z 399.2 [M + H]⁺; ¹H NMR (500 MHz, METHANOL-d4) δ 8.34-8.31 |
| | (m, 2H, including formic acid), 7.94 (t, J = 7.7 Hz, 1H), 7.85 (t, J = 7.6 Hz, |
| | 1H), 7.54 (d, J = 8.2 Hz, 1H), 7.38 (d, J = 7.8 Hz, 1H), 7.27 (d, J = 7.8 Hz, 1H), |
| | 7.22 (s, 1H), 6.74 (td, J = 74.8, 13.9 Hz, 1H), 4.71-4.63 (m, 1H), 3.96-3.72 |
| | (m, 1H), 3.56-3.39 (m, 1H), 3.16-2.94 (m, 2H), 2.91 (s, 3H), 2.50 (s, 3H), |
| | 2.30-2.14 (m, 2H), 2.03-1.83 (m, 2H); 1H not observed (NH) |
| I-136 | MS m/z 359.3 [M + H]⁺; ¹H NMR (CD₃OD) δ: 9.65 (s, 1H), 8.80 (d, J = 5.6 Hz, |
| | 1H), 7.69 (s, 1H), 7.58 (d, J = 8.0 Hz, 1H), 7.48 (d, J = 7.9 Hz, 1H), 7.38 (d, J = 5.8 |
| | Hz, 1H), 4.61-4.71 (m, 1H), 3.60-3.82 (m, 1H), 3.23-3.38 (m, 1H), 2.93-3.10 |
| | (m, 2H), 2.79 (s, 3H), 2.40 (s, 3H), 1.98-2.22 (m, 2H), 1.77-1.94 (m, 2H) |
| I-137 | MS m/z 403.2 [M + H]⁺; ¹H NMR (MeOH-d4) δ: 9.72 (s, 1H), 8.87 (d, J = 5.5 |
| | Hz, 1H), 7.97 (s, 2H), 7.75-7.80 (m, 2H), 7.55-7.63 (m, 2H), 4.60-4.70 (m, 1H), |
| | 3.15-3.25 (m, 1H), 2.70-2.80 (m, 1H), 2.28-2.48 (m, 5H), 2.05-2.15 (m, 1H), |
| | 1.90-1.98 (m, 1H), 1.60-1.85 (m, 2H). Two exchangeable protons are not seen. |
| I-145 | MS m/z 456.0 [M + H]⁺; ¹H NMR (CD₃OD) δ: 9.77 (br d, J = 2.6 Hz, 1H), 8.97 (d, |
| | J = 5.6 Hz, 1H), 8.89 (d, J = 5.6 Hz, 1H), 7.78 (d, J = 5.8 Hz, 1H), 7.67 (d, J = 8.5 Hz, |
| | 1H), 7.30 (d, J = 5.6 Hz, 1H), 5.70 (br s, 1H), 4.68-4.83 (m, 1H), 4.37-4.61 (m, |
| | 2H), 3.78 (br s, 1H), 3.34-3.56 (m, 2H), 2.96-3.13 (m, 2H), 2.88 (s, 3H), 2.15- |
| | 2.32 (m, 2H), 1.85-2.09 (m, 2H) |
| I-157 | MS m/z 378.9 [M + H]⁺; ¹H NMR (CD₃OD) δ: 9.80 (s, 1H), 8.97 (d, J = 5.8 Hz, |
| | 1H), 8.11 (d, J = 2.1 Hz, 1H), 7.90-7.99 (m, 1H), 7.76 (d, J = 8.4 Hz, 1H), 7.56 |
| | (dd, J = 5.8, 0.8 Hz, 1H), 4.73-4.85 (m, 1H), 3.71-3.88 (m, 1H), 3.35-3.45 (m, |
| | 1H), 2.99-3.19 (m, 2H), 2.90 (s, 3H), 2.16-2.32 (m, 2H), 1.91-2.05 (m, 2H) |
| I-158 | MS m/z 428.7 [M + H]⁺; ¹H NMR (DMSO-d₆) δ: 9.74 (s, 1H), 8.76 (br d, J = 5.3 |
| | Hz, 1H), 7.57-7.63 (m, 2H), 7.41 (br d, J = 7.3 Hz, 1H), 7.35 (br d, J = 5.0 Hz, |
| | 1H), 6.99 (br d, J = 5.0 Hz, 1H), 4.33-4.47 (m, 1H), 2.95-3.13 (m, 1H), 2.63-2.70 |
| | (m, 1H), 2.04-2.22 (m, 4H), 1.80-2.00 (m, 3H), 1.64-1.78 (m, 1H), 1.48-1.62 |
| | (m, 1H), 1.41 (br dd, J = 11.8, 0.8 Hz, 1H), 1.03 (br d, J = 7.3 Hz, 2H), 0.69-0.91 |
| | (m, 2H) |
| I-170 | MS m/z 388.2, 390.0 [M + H]⁺; ¹H NMR (400 MHz, DMSO-d6) δ 10.62 (s, 1H), |
| | 9.80 (s, 1H), 8.51(d, J = 5.6 Hz, 1H), 7.69 (d, J = 7.6 Hz, 1H), 7.22 (d, J = 5.2 |
| | Hz, 1H), 7.03 (dd, J = 9.2 Hz, 2.0 Hz, 1H), 6.90 (s, 1H), 4.49-4.43 (m, 1H), 3.10 |
| | (d, J = 4.0 Hz, 1H), 2.73 (d, J = 10.4 Hz, 1H), 2.23 (s, 3H), 2.03-1.94 (m, 3H), |
| | 1.79-1.76 (m, 1H), 1.66-1.57 (m, 1H), 1.52-1.45 (m, 1H). |
| I-172 | MS m/z 371.2 [M + H]⁺; ¹H NMR (400 MHz, DMSO-d6) δ 14.43 (s, 1H), 9.87 (s, |
| | 1H), 9.22(d, J = 5.6 Hz, 1H), 9.03 (d, J = 5.6 Hz, 1H), 8.31 (d, J = 2.4 Hz, 1H), |
| | 8.06 (d, J = 7.6 Hz, 1H), 7.61 (d, J = 2.0 Hz, 1H), 4.47-4.45 (m, 1H), 3.08 (d, J = |
| | 8.8 Hz, 1H), 2.73 (d, J = 11.6 Hz, 1H), 2.23 (s, 3H), 2.03-1.91 (m, 3H), 1.81- |
| | 1.76 (m, 1H), 1.64-1.61 (m, 1H), 1.54-1.47 (m, 1H). |
| I-173 | MS m/z 447.6 [M + H]⁺; ¹H NMR (400 MHz, METHANOL-d4) δ = 9.74 (s, 1H), |
| | 8.87 (d, J = 5.6 Hz, 1H), 8.08 (s, 1H), 8.06-8.01 (m, 1H), 7.89 (dd, J = 1.4, 7.9 |
| | Hz, 1H), 7.60 (d, J = 7.9 Hz, 1H), 7.46 (d, J = 5.8 Hz, 1H), 7.39 (s, 1H), 5.25- |
| | 5.12 (m, 2H), 4.71-4.62 (m, 1H), 3.31 (s, 3H), 2.96-2.81 (m, 1H), 2.57-2.42 |
| | (m, 6H), 2.24-2.06 (m, 1H), 2.03-1.92 (m, 1H), 1.89-1.68 (m, 2H); NH wasn't |
| | observed |
| I-176 | MS m/z 402.9 [M + H]⁺; ¹H NMR (400 MHz, METHANOL-d4) δ = 9.71 (s, 1H), |
| | 8.87 (d, J = 5.8 Hz, 1H), 8.05 (s, 1H), 7.73-7.66 (m, 2H), 7.59-7.52 (m, 2H), |
| | 7.36 (s, 1H), 4.75-4.57 (m, 1H), 2.96-2.82 (m, 1H), 2.61-2.42 (m, 6H), 2.14 |
| | (br s, 1H), 2.04-1.93 (m, 1H), 1.90-1.67 (m, 2H); OH and NH wasn't observed |
| I-179 | MS m/z 368.2 [M + H]⁺; ¹H NMR (400 MHz, DMSO-d6) δ 9.87 (s, 1H), 9.79 (s, |
| | 1H), 8.84 (d, J = 5.6 Hz, 1H), 7.62 (d, J = 7.6 Hz, 1H), 7.18 (d, J = 5.6 Hz, 1H), |
| | 6.67-6.64 (m, 2H), 4.46-4.42 (m, 1H), 3.11 (d, J = 7.6 Hz, 1H), 2.73 (d, J = |
| | 10.8 Hz, 1H), 2.33 (s, 3H), 2.23 (s, 3H), 2.03-1.95 (m, 3H), 1.79-1.76 (m, |
| | 1H), 1.66-1.57 (m, 1H), 1.52-1.45 (m, 1H). |
| I-188 | MS m/z 460.4 [M + H]⁺; ¹H NMR (400 MHz, METHANOL-d4) δ = 9.77-9.73 |
| | (m, 1H), 8.88 (d, J = 5.6 Hz, 1H), 7.69 (d, J = 7.8 Hz, 1H), 7.59-7.49 (m, 1H), |
| | 7.49-7.40 (m, 1H), 7.06 (s, 1H), 5.47-5.40 (m, 1H), 4.98-4.89 (m, 2H), 4.75- |
| | 4.60 (m, 1H), 4.46 (br s, 1H), 4.40-4.27 (m, 1H), 3.30-3.19 (m, 1H), 2.88- |
| | 2.77 (m, 1H), 2.46 (s, 5H), 2.20-2.06 (m, 1H), 2.00-1.94 (m, 1H), 1.89-1.67 |
| | (m, 2H); NH wasn't observed |
| I-189 | MS m/z 468.2 [M + H]⁺; ¹H NMR (400 MHz, CD₃OD) δ 9.7(s, 1H), 8.8(d, J = 5.6 |
| | Hz 1H), 8.0(s, 1H), 7.8(d, J = 8 Hz, 1H), 7.7(d, J = 8 Hz, 1H), 7.6(s, 1H), 7.6(s, 1H), |
| | 7.2(d, J = 5.6 Hz, 1H), 7.0(d, J = 13.6 Hz, 1H), 4.7(m, 1H), 4.0(d, J = 5.6 Hz, 1H), 3.7(d, |
| | J = 5.2 Hz, 3H), 3.6(m, 1H), 3.0(m, 1H), 2.98(d, J = 6.8 Hz, 1H), 2.91 (d, J = 3.6 |
| | Hz, 1H), 2.3 (dd, J = 52.4 Hz, 2H), 2.0-1.8(m, 2H). |
| I-193 | MS m/z 454.1 [M + H]⁺; ¹H NMR (400 MHz, DMSO-d6) δ 9.85 (s, 1H), 8.86 (d, |
| | J = 5.6 Hz, 1H), 8.11 (s, 1H), 7.93 (d, J = 8.0 Hz, 1H), 7.81 (d, J = 7.6 Hz, 1H), 7.20 |
| | (d, J = 5.6 Hz, 1H), 4.50-4.48 (m, 1H), 3.10 (d, J = 8.8 Hz, 1H), 2.73 (d, J = 10.8 |
| | Hz, 1H), 2.23 (s, 3H), 2.04-1.91 (m, 3H), 1.79-1.76(m, 1H), 1.63-1.60 (m, 1H), |
| | 1.49-1.47(m, 1H). |
| I-195 | MS m/z 351.2 [M + H]⁺; ¹H NMR (400 MHz, DMSO-d6) δ 13.87 (s, 1H), 9.86 (s, |
| | 1H), 9.43(d, J = 5.6 Hz, 1H), 9.03 (d, J = 5.6 Hz, 1H), 8.15 (d, J = 1.2 Hz, 1H), |
| | 7.96 (d, J = 7.2 Hz, 1H), 7.28 (d, J = 1.2 Hz, 1H), 4.46-4.45(m, 1H), 3.11-3.07 |
| | (m, 1H), 2.76-2.73 (m, 1H), 2.35 (s, 3H), 2.24 (s, 3H), 2.04-1.93 (m, 3H), 1.80- |
| | 1.76 (m, 1H), 1.64-1.61 (m, 1H), 1.51-1.47 (m, 1H). |
| I-196 | MS m/z 394.2 [M + H]⁺; ¹H NMR (400 MHz, DMSO-d6) δ 9.89 (br s, 1H), 9.79 |
| | (s, 1H), 8.84 (d, J = 5.2 Hz, 1H), 7.64 (d, J = 7.2 Hz, 1H), 7.18 (d, J = 5.2 Hz, |
| | 1H), 6.58 (s, 1H), 6.53 (d, J = 10.8 Hz, 1H), 4.46-4.45 (m, 1H), 3.11 (d, J = |
| | 8.0 Hz, 1H), 2.73 (d, J = 10.4 Hz, 1H), 2.23 (s, 3H), 2.03-1.95 (m, 4H), 1.79- |
| | 1.76 (m, 1H), 1.63-1.60 (m, 1H), 1.51-1.42 (m, 1H), 1.01 (d, J = 6.4 Hz, 2H), |
| | 0.73 (d, J = 4.0 Hz, 2H). |
| I-200 | MS m/z 414.7 [M + H]⁺; ¹H NMR (400 MHz, METHANOL-d4) δ = 9.60 (s, 1H), |
| | 8.81-8.72 (m, 3H), 8.05-7.91 (m, 2H), 7.50-7.45 (m, 1H), 7.45-7.39 (m, |
| | 1H), 7.29 (t, J = 4.9 Hz, 1H), 4.63-4.44 (m, 1H), 3.11-2.96 (m, 1H), 2.60 (br |
| | s, 1H), 2.29-2.19 (m, 5H), 1.99 (br s, 1H), 1.79 (br dd, J = 4.1, 9.2 Hz, 1H), |
| | 1.72-1.50 (m, 2H); NH and OH wasn't observed |
| I-201 | MS m/z 432.0 [M + H]⁺; ¹H NMR (400 MHz, METHANOL-d4) δ = 8.23 (d, J = |
| | 8.3 Hz, 1H), 7.97 (dt, J = 1.1, 7.7 Hz, 1H), 7.85 (dt, J = 1.0, 7.7 Hz, 1H), 7.63- |
| | 7.54 (m, 2H), 7.52-7.44 (m, 2H), 4.08 (br t, J = 12.1 Hz, 2H), 3.80 (s, 3H), |
| | 3.23-3.05 (m, 2H), 2.60-2.50 (m, 1H), 2.43 (s, 6H), 2.14 (br d, J = 12.3 Hz, |
| | 2H), 2.00-1.81 (m, 2H) |
| I-202 | MS m/z 464.3 [M + H]⁺; ¹H NMR (CD₃OD) δ: 9.58 (s, 1H), 8.60 (d, J = 5.6 Hz, |
| | 1H), 7.78-7.91 (m, 3H), 7.08-7.20 (m, 6H), 4.56-4.67 (m, 1H), 3.37 (s, 1H), |
| | 3.04-3.24 (m, 1H), 2.72 (s, 1H), 2.37 (d, J = 6.5 Hz, 3H), 2.26-2.33 (m, 1H), 2.08 |
| | (br dd, J = 12.0, 7.6 Hz, 1H), 1.85-1.95 (m, 1H), 1.77 (td, J = 6.6, 2.8 Hz, 1H), |
| | 1.66 (br s, 1H) |
| I-205 | MS m/z 362.6 [M + H]⁺; ¹H NMR (CD₃OD) δ: 9.79 (s, 1H), 8.89 (d, J = 5.6 Hz, |
| | 1H), 7.22 (d, J = 5.5 Hz, 1H), 7.09 (s, 2H), 4.78-4.86 (m, 1H), 3.74-4.04 (m, |
| | 1H), 3.37-3.50 (m, 1H), 3.07-3.21 (m, 2H), 2.90-2.94 (m, 3H), 2.40 (s, 3H), |
| | 2.15-2.31 (m, 4H), 1.93 (d, J = 3.1 Hz, 6H) |
| I-207 | MS m/z 404.7 [M + H]⁺; ¹H NMR (CD₃OD) δ: 9.77 (s, 1H), 8.84 (d, J = 5.6 Hz, |
| | 1H), 7.41 (s, 1H), 7.29 (d, J = 5.5 Hz, 1H), 7.22-7.26 (m, 1H), 7.18 (dd, J = 7.8, |
| | 1.9 Hz, 1H), 4.62-4.73 (m, 1H), 3.29 (br s, 1H), 3.02 (dt, J = 13.8, 6.9 Hz, 1H), |
| | 2.87 (br d, J = 8.0 Hz, 1H), 2.55-2.67 (m, 1H), 2.48 (s, 4H), 2.07-2.18 (m, 1H), |
| | 1.89-2.02 (m, 1H), 1.70-1.89 (m, 2H), 1.33 (d, J = 6.9 Hz, 6H), 1.17 (br d, J = 6.8 |
| | Hz, 3H), 1.09 (d, J = 6.8 Hz, 3H) |
| I-208 | MS m/z 408.6 [M + H]⁺; ¹H NMR (400 MHz, CD₃OD)) δ 9.73 (d, J = 1.0 Hz, 1H), |
| | 8.86 (d, J = 5.7 Hz, 1H), 7.31 (d, J = 5.7 Hz, 1H), 7.12 (dd, J = 8.8, 6.4 Hz, 1H), |
| | 7.02 (t, J = 8.7 Hz, 1H), 4.72-4.59 (m, 1H), 3.56 (s, 3H), 3.36-3.32 (m, 2H, |
| | 3.00-2.72 (m, 1H), 2.63-2.35 (m, 5H), 2.28-2.06 (m, 2H), 2.05-1.90 (m, |
| | 1H), 1.88-1.61 (m, 2H), 1.13-0.96 (m, 2H), 0.91-0.77 (m, 1H), 0.77-0.64 |
| | (m, 1H). |
| I-209 | MS m/z 388.5 [M + H]⁺; ¹H NMR (400 MHz, CD₃OD) δ 9.63 (s, 1H), 8.81 (s, 1H), |
| | 8.52 (s, 1H), 7.51 (ddd, J = 9.1, 6.1, 1.4 Hz, 1H), 7.36 (d, J = 5.6 Hz, 1H), 6.83- |
| | 6.71 (m, 1H), 4.76-4.65 (m, 1H), 3.52-3.36 (m, 1H), 3.08-2.94 (m, 1H), 2.91- |
| | 2.65 (m, 2H), 2.62 (s, 3H), 2.22-1.99 (m, 2H), 1.94-1.72 (m, 2H). |
| I-210 | MS m/z 394.6 [M + H]⁺; ¹H NMR (400 MHz, CD₃OD) δ 9.72 (s, 1H), 8.87 (dd, |
| | J = 5.7, 2.1 Hz, 1H), 8.54 (s, 1H), 7.44-7.39 (m, 1H), 7.36-7.30 (m, 1H), 6.76 |
| | (dd, J = 10.0, 8.2 Hz, 1H), 5.06-4.93 (m, 1H), 4.71-4.61 (m, 1H), 3.53-3.35 |
| | (m, 1H), 2.95-2.79 (m, 2H), 2.57-2.38 (m, 5H), 2.22-2.05 (m, 1H), 2.02- |
| | 1.90 (m, 1H), 1.88-1.64 (m, 2H), 1.43-1.31 (m, 3H). |
| I-211 | MS m/z 388.5 [M + H]⁺; ¹H NMR (400 MHz, CD₃OD) δ 9.02 (dd, J = 4.3, 1.5 Hz, |
| | 1H), 8.73 (d, J = 8.5 Hz, 1H), 8.53 (s, 1H), 7.82 (dd, J = 8.4, 4.4 Hz, 1H), 7.46 |
| | (dd, J = 8.9, 6.0 Hz, 1H), 6.75 (t, J = 8.7 Hz, 1H), 4.69-4.58 (m, 1H), 3.43- |
| | 3.32 (m, 1H), 3.03-2.88 (m, 1H), 2.77-2.44 (m, 5H), 2.21-2.06 (m, 1H), 2.04- |
| | 1.93 (m, 1H), 1.91-1.65 (m, 2H). |
| I-216 | MS m/z 348.5 [M + H]⁺; ¹H NMR (CD₃OD) δ: 9.75 (s, 1H), 8.84 (d, J = 5.6 Hz, |
| | 1H), 7.30 (d, J = 5.6 Hz, 1H), 7.24 (s, 1H), 7.19 (s, 2H), 4.62-4.73 (m, 1H), 3.22- |
| | 3.45 (m, 2H), 2.81-2.97 (m, 1H), 2.43-2.55 (m, 5H), 2.42 (s, 3H), 2.08-2.15 (m, |
| | 1H), 2.07 (s, 3H), 1.97 (br dd, J = 9.2, 4.6 Hz, 1H), 1.71-1.90 (m, 2H) |
| I-217 | MS m/z 359.5 [M + H]⁺; ¹H NMR (CD₃OD) δ: 9.78 (s, 1H), 8.92 (d, J = 5.8 Hz, |
| | 1H), 8.37 (s, 1H), 7.86 (s, 1H), 7.79 (d, J = 7.5 Hz, 1H), 7.54 (d, J = 7.9 Hz, 1H), |
| | 7.32 (dd, J = 5.7, 0.7 Hz, 1H), 4.71-4.80 (m, 1H), 3.71-3.93 (m, 1H), 3.42 (br d, |
| | J = 12.1 Hz, 1H), 2.96-3.13 (m, 2H), 2.90 (s, 3H), 2.20-2.29 (m, 2H), 2.19 (s, |
| | 3H), 1.88-2.06 (m, 2H) |
| I-218 | MS m/z 382.5 [M + H]⁺; ¹H NMR (CD₃OD) δ: 9.80 (s, 1H), 8.91 (d, J = 5.6 Hz, |
| | 1H), 8.40 (s, 2H), 7.31 (s, 2H), 7.22 (dd, J = 5.6, 0.8 Hz, 1H), 4.71-4.87 (m, 1H), |
| | 3.76-4.00 (m, 1H), 3.35-3.52 (m, 1H), 3.07-3.20 (m, 2H), 2.93 (s, 3H), 2.14- |
| | 2.34 (m, 2H), 1.89-2.05 (m, 8H) |
| I-225 | MS m/z 399.6 [M + H]⁺; ¹H NMR (400 MHz, CD₃OD) δ 8.52 (s, 1H), 8.29 (d, J = |
| | 8.2 Hz, 1H), 7.94-7.86 (m, 1H), 7.82 (t, J = 7.6 Hz, 1H), 7.67 (d, J = 8.1 Hz, |
| | 1H), 7.25 (d, J = 7.8 Hz, 1H), 7.00 (dd, J = 7.8, 1.5 Hz, 1H), 6.96 (d, J = 1.5 Hz, |
| | 1H), 4.67-4.57 (m, 1H), 3.69-3.51 (m, 1H), 3.22-3.09 (m, 1H), 2.92-2.73 |
| | (m, 2H), 2.70 (s, 3H), 2.26-2.13 (m, 1H), 2.12-2.01 (m, 1H), 1.98-1.70 (m, |
| | 2H), 1.57-1.44 (m, 1H), 0.98-0.86 (m, 2H), 0.84-0.71 (m, 2H). |
| I-227 | MS m/z 373.5 [M + H]⁺; ¹H NMR (400 MHz, CD₃OD) δ 8.54 (s, 1H), 8.28 (d, J = |
| | 8.2 Hz, 1H), 7.88 (t, J = 7.6 Hz, 1H), 7.81 (t, J = 7.6 Hz, 1H), 7.67 (d, J = 8.1 Hz, |
| | 1H), 7.26 (d, J = 7.7 Hz, 1H), 7.01 (d, J = 7.8 Hz, 1H), 6.98 (s, 1H), 4.68-4.54 |
| | (m, 1H), 3.59-3.41 (m, 1H), 3.14-2.97 (m, 1H), 2.82-2.64 (m, 2H), 2.62 (s, |
| | 3H), 2.20-2.09 (m, 1H), 2.09-1.98 (m, 4H), 1.92-1.70 (m, 2H). |
| I-232 | MS m/z 468.2 [M + H]⁺; ¹H NMR (400 MHz, CD₃OD) δ 9.74 (s, 1 H), 8.79 (d, J = |
| | 5.6 Hz 1 H), 8.23 (s, 1 H), 7.90 (d, J = 7.6 Hz, 1 H), 7.77 (d, J = 8.0 Hz, 1 H), |
| | 7.33 (d, J = 2.4 Hz, 1 H), 7.22 (d, J = 5.6 Hz, 1 H), 5.97 (d, J = 8.8 Hz, 1 H), 4.77- |
| | 4.75 (m, 1 H), 4.05-4.03 (m, 1 H), 3.64 (s, 3 H), 3.64-3.60 (m, 1 H), 3.00 (d, J = |
| | 5.2 Hz, 3 H), 3.10-2.95 (m, 2 H), 2.39-2.36 (m, 1 H), 2.24-2.21 (m, 1 H), |
| | 2.06-1.88 (m, 2 H) |
| I-240 | MS m/z 465.2 [M + H]⁺; ¹H NMR (400 MHz, DMSO-d6) δ 9.70 (s, 1H), 8.68 (d, |
| | J = 5.6 Hz, 1H), 8.33-8.27 (m, 2H), 8.02-7.96 (m, 2H), 7.83 (d, J = 7.9 Hz, |
| | 1H), 7.68 (d, J = 7.4 Hz, 1H), 7.60 (d, J = 7.8 Hz, 1H), 7.21 (dd, J = 7.8, 4.9 Hz, |
| | 1H), 7.11 (d, J = 5.5 Hz, 1H), 4.44 (s, 1H), 3.10 (s, 1H), 2.75 (d, J = 10.5 Hz, |
| | 1H), 2.25 (s, 3H), 1.97 (s, 3H), 1.75 (s, 1H), 1.61 (d, J = 12.0 Hz, 1H), 1.44 (s, |
| | 1H). |
| I-241 | MS m/z 409.5, 411.4 [M + H]⁺; ¹H NMR (400 MHz, DMSO-d6) δ 7.50-7.39 |
| | (m, 3H), 7.28 (t, J = 73.7 Hz, 1H), 6.08 (d, J = 7.9 Hz, 1H), 4.32-4.18 (m, |
| | 1H), 3.00-2.91 (m, 1H), 2.74 (t, J = 7.8 Hz, 2H), 2.67 (t, J = 8.0 Hz, 2H), 2.18 |
| | (s, 3H), 2.04-1.94 (m, 3H), 1.94-1.81 (m, 3H), 1.75-1.65 (m, 1H), 1.62- |
| | 1.48 (m, 1H), 1.43-1.30 (m, 1H) |
| I-244 | MS m/z 389.5 [M + H]⁺; ¹H NMR (400 MHz, METHANOL-d4) δ 8.53 (s, 1H, |
| | formic acid), 7.34-7.28 (m, 1H), 7.22-7.15 (m, 1H), 7.11 (s, 1H), 6.73 (t, J = |
| | 75.04, 1H), 4.50-4.40 (m, 1H), 3.50-3.39 (m, 1 H), 3.13-3.00 (m, 1 H), |
| | 2.87 (t, J = 7.63 Hz, 2H), 2.79 (t, J = 8.25 Hz, 2 H), 2.72-2.54 (m, 5H), 2.43 (s, |
| | 3H), 2.20-1.92 (m, 4H), 1.91-1.75 (m, 1H), 1.75-1.58 (m, 1H). 1H not |
| | observed (NH) |
| I-246 | MS m/z 415.5 [M + H]⁺; ¹H NMR (400 MHz, METHANOL-d4) δ 7.30 (d, J = |
| | 7.9 Hz, 1H), 7.07 (d, J = 7.8 Hz, 1H), 6.99 (s, 1H), 6.75 (t, J = 74.04 Hz, 1H), |
| | 4.57-4.45 (m, 1H), 3.79-3.58 (m, 1H), 3.43-3.34 (m, 1H), 3.10-2.92 (m, |
| | 2H), 2.92-2.84 (m, 5H), 2.80 (t, J = 7.6 Hz, 2H), 2.20-2.08 (m, 4H), 2.06- |
| | 1.97 (m, 1H), 1.97-1.84 (m, 1H), 1.84-1.68 (m, 1H), 1.12-1.03 (m, 2H), |
| | 0.78 (br s, 2H); 1H missing (NH) |
| I-247 | MS m/z 425.9 [M + H]⁺; ¹H NMR (400 MHz, DMSO-d6) δ 9.79 (s, 1 H), 8.84 |
| | (d, J = 5.63 Hz, 1 H), 8.19 (br s, 1 H, formic acid), 7.67 (d, J = 7.38 Hz, 1 H), |
| | 7.38 (d, J = 7.88 Hz, 1 H), 7.22 (d, J = 5.63 Hz, 1 H), 7.16 (t, J = 74.04 Hz, 1 |
| | H), 7.12 (d, J = 8.25 Hz, 1 H), 7.08 (s, 1 H), 4.53-4.38 (m, 1 H), 3.17-3.07 |
| | (m, 1 H), 2.75 (d, J = 10.76 Hz, 1 H), 2.25 (s, 3 H), 2.12-1.93 (m, 4 H), 1.85- |
| | 1.73 (m, 1 H), 1.68-1.55 (m, 1 H), 1.53-1.41 (m, 1 H), 1.10-1.02 (m, 2 H), |
| | 0.86-0.79 (m, 2 H) |
| I-248 | MS m/z 409.9 [M + H]⁺; ¹H NMR (400 MHz, DMSO-d6) δ 9.81 (s, 1 H), 8.85 |
| | (d, J = 5.63 Hz, 1 H), 8.21 (br s, 1 H, formic acid), 7.71 (d, J = 7.50 Hz, 1 H), |
| | 7.54 (s, 1 H), 7.39-7.32 (m, 2 H), 7.22 (d, J = 5.63 Hz, 1 H), 6.84 (t, J = 54.66 |
| | Hz, 1 H), 4.57-4.40 (m, 1 H), 3.18-3.08 (m, 1 H), 2.76 (d, J = 11.38 Hz, 1 H), |
| | 2.25 (s, 3 H), 2.19-2.08 (m, 1 H), 2.07-1.93 (m, 3 H), 1.85-1.74 (m, 1 H), |
| | 1.70-1.55 (m, 1 H), 1.54-1.40 (m, 1 H), 1.13-1.03 (m, 2 H), 0.86-0.78 (m, |
| | 2 H) |
| I-289 | MS m/z 422.4 [M + H]⁺; ¹H NMR (400 MHz, DMSO-d6) δ 9.84 (s, 1 H), 8.85 |
| | (d, J = 5.4 Hz, 1 H), 8.29-8.12 (br, 1H), 8.06 (s, 1 H, formic acid), 7.94 (d, |
| | J = 8.3 Hz, 1 H), 7.79 (d, J = 7.3 Hz, 1H), 7.63 (d, J = 7.9 Hz, 1H), 7.14 (d, J = |
| | 5.4 Hz, 1H), 4.45-4.58 (m, 1 H), 3.07-3.23 (m, 1 H), 2.80 (m, 1 H), 2.29 (s, 3 |
| | H), 1.98-2.22 (m, 3 H), 1.88-1.75 (m, 1H), 1.64 (m, 1H), 1.51 (m, 1H). |
| I-298 | MS m/z 402.5 [M + H]⁺; ¹H NMR (DMSO-d₆) δ: 9.97-9.69 (m, 1H), 9.05- |
| | 8.68 (m, 1H), 7.88-7.77 (m, 1H), 7.74-7.57 (m, 2H), 7.45 (s, 1H), 7.16- |
| | 6.93 (m, 1H), 4.66-4.35 (m, 1H), 3.12 (d, J = 1.3 Hz, 2H), 2.76 (dd, J = 2.6, |
| | 1.3 Hz, 1H), 2.72-2.67 (m, 1H), 2.51 (s, 1H), 2.26 (s, 3H), 1.99 (d, J = 18.0 |
| | Hz, 3H), 1.78 (d, J = 2.6 Hz, 1H), 1.72-1.57 (m, 1H), 1.50 (s, 1H). |
| I-307 | MS m/z 364.3 [M + H]⁺; ¹H NMR (400 MHz, DMSO-d6) δ 9.76 (s, 1H), 9.63 (br |
| | s, 1H), 8.84 (s, 1H), 8.18 (s, 1H, formic acid), 7.54 (d, J = 7.3 Hz, 1H), 7.31 (d |
| | J = 5.88 Hz, 1H), 7.22 (d, J = 7.5 Hz, 1H), 6.86-6.79 (m, 2H), 4.52-4.39 (m, |
| | 1H), 3.16-3.07 (m, 1H), 2.81-2.70 (m, 1H), 2.63 (q, J = 7.5 Hz, 2H), 2.30- |
| | 2.20 (m, 3H), 2.07-1.91 (m, 3H), 1.83-1.72 (m, 1H), 1.70-1.55 (m, 1H), |
| | 1.54-1.39 (m, 1H), 1.23 (t, J = 7.6 Hz, 3H) |

### Example 12

### Preparation of Compounds I-31 and I-36

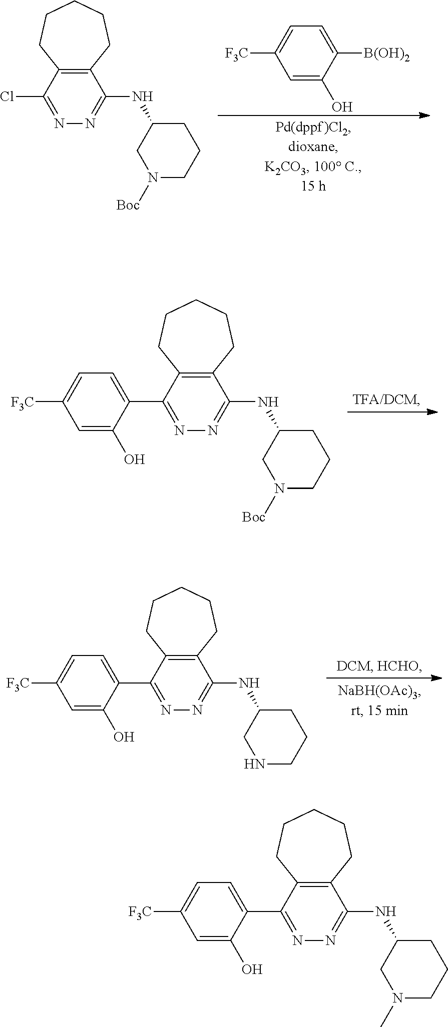 [SMILES: CC(C)(C)OC(=O)N1CCC[C@@H](Nc2nnc(Cl)c3c2CCCCC3)C1 ; OB(O)c1ccc(C(F)(F)F)cc1O ; CC(C)(C)OC(=O)N1CCC[C@@H](Nc2nnc(-c3ccc(C(F)(F)F)cc3O)c3c2CCCCC3)C1 ; Oc1cc(C(F)(F)F)ccc1-c1nnc(N[C@@H]2CCCNC2)c2c1CCCCC2 ; CN1CCC[C@@H](Nc2nnc(-c3ccc(C(F)(F)F)cc3O)c3c2CCCCC3)C1]

### Step 1: tert-Butyl (R)-3-((4-(2-hydroxy-4-(trifluoromethyl)phenyl)-6,7,8,9-tetrahydro-5H-cyclohepta[d]pyridazin-1-yl)amino)piperidine-1-carboxylate

tert-Butyl (R)-3-((4-chloro-6,7,8,9-tetrahydro-5H-cyclohepta[d]pyridazin-1-yl)amino)piperidine-1-carboxylate (Intermediate 22, 100 mg, 0.24 mmol), (2-hydroxy-4-trifluoromethyl)phenyl)boronic acid (63 mg, 0.3 mmol), Pd(dppf)Cl₂-Dichloromethane complex (20 mg, 0.024 mmol), 2M aqueous K₂CO₃ (0.3 mL, 0.6 mmol), and dioxane (0.8 mL) were heated at 100° C. for 15 hours. This was then partitioned between H₂O and DCM. The organic layer was dried over MgSO₄ and filtered. Solvent was removed under vacuum. Purification by silica gel chromatography (0-100% EtOAc in DCM) yielded tert-butyl (R)-3-((4-(2-hydroxy-4-(trifluoromethyl)phenyl)-6,7,8,9-tetrahydro-5H-cyclohepta[d]pyridazin-1-yl)amino)piperidine-1-carboxylate (108 mg, 90%).

### Step 2. (R)-2-(4-(Piperidin-3-ylamino)-6,7,8,9-tetrahydro-5H-cyclohepta[d]pyridazin-1-yl)-5-(trifluoromethyl)phenol

tert-Butyl (R)-3-((4-(2-hydroxy-4-(trifluoromethyl)phenyl)-6,7,8,9-tetrahydro-5H-cyclohepta[d]pyridazin-1-yl)amino)piperidine-1-carboxylate (106 mg, 0.21 mmol), DCM (0.8 mL), and TFA (0.2 mL) were stirred at room temperature for 1 hour. Volatiles were removed by nitrogen stream. This mixture was then partitioned between aqueous NaOH and DCM. The DCM layer was dried over MgSO₄ and was filtered. (R)-2-(4-(piperidin-3-ylamino)-6,7,8,9-tetrahydro-5H-cyclohepta[d]pyridazin-1-yl)-5-(trifluoromethyl)phenol (89 mg, 93%) was obtained from the filtrate as an off-white solid. MS m/z 407.3 [M+H]⁺; ¹H NMR (MeOH-d4) δ: 37.36 (d, J=8 Hz, 1H), 7.22 (d, J=8 Hz, 1H), 7.15 (s, 1H), 4.26-4.32 (m, 1H), 3.41-3.46 (m, 1H), 3.05-3.10 (m, 1H), 2.60-2.83 (m, 6H), 2.11-2.16 (m, 1H), 1.87-1.93 (m, 3H), 1.59-1.80 (m, 6H). Three exchangeable protons are not seen.

### Step 3: (R)-2-(4-((1-Methylpiperidin-3-yl)amino)-6,7,8,9-tetrahydro-5H-cyclohepta[d]pyridazin-1-yl)-5-(trifluoromethyl)phenol

(R)-2-(4-(Piperidin-3-ylamino)-6,7,8,9-tetrahydro-5H-cyclohepta[d]pyridazin-1-yl)-5-(trifluoromethyl)phenol (30 mg, 0.074 mmol), sodium triacetoxyborohydride (30 mg, 0.14 mmol), and DCM (0.5 mL) were stirred at room temperature. Aqueous formaldehyde (1.28 M, 0.1 mL, 0.13 mmol) was added dropwise. This was stirred vigorously at room temperature for 15 minutes. The mixture was partitioned between aqueous K₂CO₃ and DCM. The organic layer was dried over MgSO₄, filtered, and concentrated under vacuum. Purification by silica (0-70% MeON in DCM) yielded (R)-2-(4-((1-methylpiperidin-3-yl)amino)-6,7,8,9-tetrahydro-5H-cyclohepta[d]pyridazin-1-yl)-5-(trifluoromethyl)phenol (21 mg, 68%) as a white solid. MS m/z 421.9 [M+H]⁺; ¹H NMR (MeOH-d4) δ: 7.36 (d, J=8 Hz 1H), 7.22 (d, J=8 Hz 1H), 7.15 (s, 1H), 4.38-4.43 (m, 1H), 3.08-3.20 (m, 1H), 2.80-2.84 (m, 2H), 2.65-2.79 (m, 1H), 2.58-2.62 (m, 2H), 2.25-2.41 (m, 5H), 1.95-2.05 (m, 1H), 1.83-1.95 (m, 3H), 1.55-1.80 (m, 6H). Two exchangeable protons are not seen.

### Example 13

### Preparation of Compound I-47

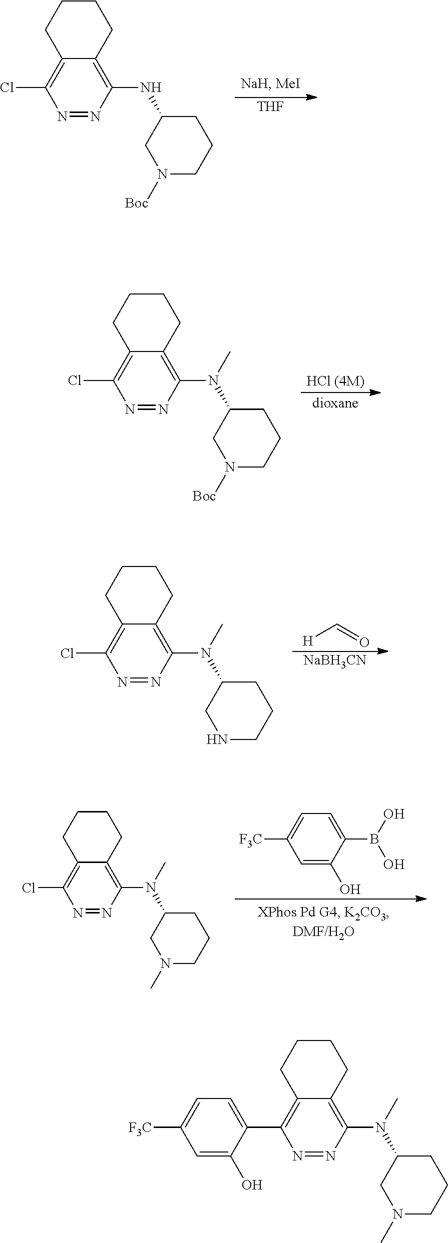

### Step 1. tert-Butyl (3R)-3-[(4-chloro-5, 6, 7, 8-tetrahydrophthalazin-1-yl)-methyl-amino]piperidine-1-carboxylate

To a solution of tert-butyl (3R)-3-[(4-chloro-5, 6, 7, 8-tetrahydrophthalazin-1-yl)amino]piperidine-1-carboxylate (prepared according to the procedure of Intermediate 19a, 1.3 g, 3.5 mmol) in 15 mL THE was added sodium hydride (218 mg, 5.4 mmol, 60 mass %) at 0° C. The mixture was stirred at rt for 1 h. Then iodomethane (774 mg, 5.4 mmol) was added and the reaction was stirred at rt for 16 h, monitored by TLC and LCMS. After reaction, the mixture was quenched by water and extracted with EA. The combined organic layers was evaporated to dryness in vacuum. The residue was purified by column chromatography (0-20% EA in PE) to afford tert-butyl (3R)-3-[(4-chloro-5, 6, 7, 8-tetrahydrophthalazin-1-yl)-methyl-amino]piperidine-1-carboxylate (500 mg, 37% Yield) as a light yellow solid. MS m/z 381.2[M+H]⁺.

### Step 2. 4-Chloro-N-methyl-N-[(3R)-3-piperidyl]-5, 6, 7, 8-tetrahydrophthalazin-1-amine

To a solution of tert-butyl (3R)-3-[(4-chloro-5, 6, 7, 8-tetrahydrophthalazin-1-yl)-methyl-amino]piperidine-1-carboxylate (500 mg, 1.3 mmol) in 4 mL DCM was added HCl (2 mL, 4M in dioxane). This mixture was stirred at r.t for 3 h, monitored by TLC and LCMS. The solution was concentrated to give crude 4-chloro-N-methyl-N-[(3R)-3-piperidyl]-5, 6, 7, 8-tetrahydrophthalazin-1-amine (350 mg, 95% Yield) as a light yellow solid. MS m/z 281.2 [M+H]⁺.

### Step 3. 4-Chloro-N-methyl-N-[(3R)-1-methyl-3-piperidyl]-5, 6, 7, 8-tetrahydrophthalazin-1-amine

To a solution of 4-chloro-N-methyl-N-[(3R)-3-piperidyl]-5, 6, 7, 8-tetrahydrophthalazin-1-amine (5, 350 mg, 1.25 mmol) and formaldehyde (50 mg, 1.67 mmol) in MeOH (5 mL) was added sodium cyanoborohydride (86 mg, 1.37 mmol). This mixture was stirred at r.t for 16 h, monitored by TLC and LCMS. After reaction, the mixture was quenched by NH₃H₂O (7 mol/L in MeOH) and stirred at r.t for 1 h. The solution was concentrated and purified by column chromatography (0-5% MeOH in DCM) to afford 4-chloro-N-methyl-N-[(3R)-1-methyl-3-piperidyl]-5, 6, 7, 8-tetrahydrophthalazin-1-amine (200 mg, 54% Yield). MS m/z 295.1[M+H]⁺.

### Step 4. 2-[4-[Methyl-[(3R)-1-methyl-3-piperidyl]amino]-5,6,7,8-tetrahydrophthalazin-1-yl]-5-(trifluoromethyl)phenol

To a solution of 4-chloro-N-methyl-N-[(3R)-1-methyl-3-piperidyl]-5, 6, 7, 8-tetrahydrophthalazin-1-amine (100 mg, 0.34 mmol) in 2 mL DMF and 0.5 mL H₂O was added [2-hydroxy-4-(trifluoromethyl)phenyl]boronic acid (80 mg, 0.39 mmol), XPhos Pd G4 (30 mg, 0.03 mmol) and potassium carbonate (94 mg, 0.68 mmol). The mixture was stirred at 90° C. under N₂ atmosphere for 16 h, monitored by TLC and LCMS. After reaction, the mixture was concentrated and purified by prep-HPLC to give 2-[4-[methyl-[(3R)-1-methyl-3-piperidyl]amino]-5, 6, 7, 8-tetrahydrophthalazin-1-yl]-5-(trifluoromethyl)phenol (30 mg, 21% Yield) as a light yellow solid. MS m/z 421.0 [M+H]⁺. ¹H NMR (400 MHz, MeOD-d4) δ: 7.36 (d, J=8 Hz, 1H), 7.24 (d, J=8 Hz, 1H), 7.17 (s, 1H), 3.58-3.54 (m, 1H), 3.13-3.09 (m, 1H), 2.93-2.91 (m, 4H), 2.74-2.73 (m, 2H), 2.54-2.52 (m, 2H), 2.42-2.39 (m, 4H), 2.08-2.06 (m, 1H), 1.97-1.55 (m, 8H).

### Example 14

### Preparation of Compounds I-20 and I-48

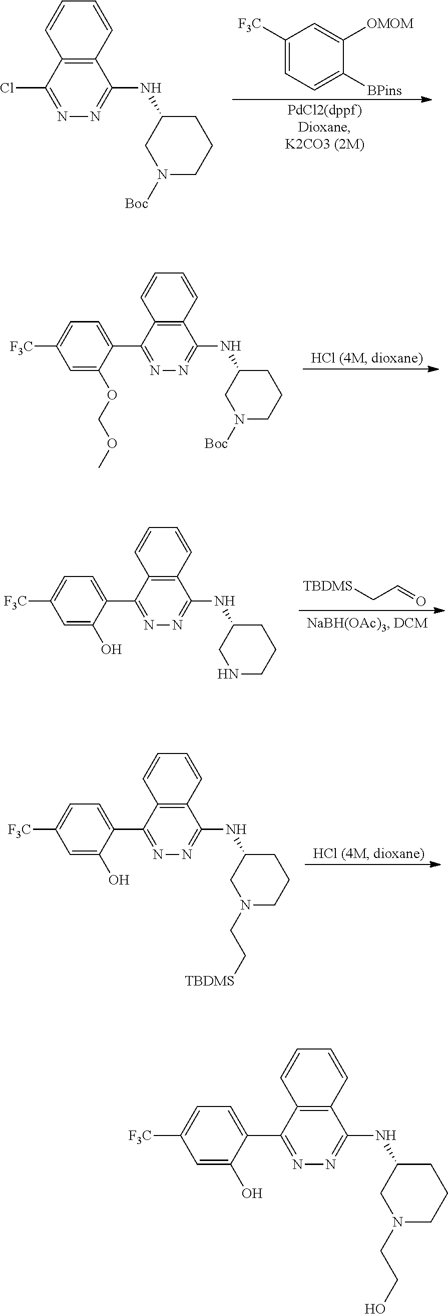

### Step 1. tert-Butyl (3R)-3-[[4-[2-(methoxymethoxy)-4-(trifluoromethyl)phenyl]phthalazin-1-yl]amino]piperidine-1-carboxylate

A mixture of tert-butyl (3R)-3-[(4-chlorophthalazin-1-yl)amino]piperidine-1-carboxylate (prepared according to the procedure of Intermediate 19a, 120 mg, 0.33 mmol, 1.0 eq.), 2-[2-(methoxymethoxy)-4-(trifluoromethyl)phenyl]-4,4,5,5-tetramethyl-1,3,2-dioxaborolane (143 mg, 0.43 mmol, 1.3 eq.) and Xphos Pd G3 (23 mg, 0.08 eq.) in K₂CO₃ (2M) in H₂O (0.5 mL, 3.0 eq.) and dioxane (1.3 mL) were bubbled with Ar. The sealed tube was then heated at 90° C. for 12 hr. After cooling, the reaction was diluted with EtOAc and water. The organic phase was washed with H₂O followed by brine. The organic phase was dried over MgSO₄, filtered, and concentrated in vacuo. The crude material was purified by flash column chromatography on silica gel eluting with 0-50% EtOAc in DCM to provide tert-butyl (3R)-3-[[4-[2-(methoxymethoxy)-4-(trifluoromethyl)phenyl]phthalazin-1-yl]amino]piperidine-1-carboxylate (141 mg, 80% yield). MS m/z 533.8 [M+H]⁺.

### Step 2. 2-[4-[[(3R)-3-Piperidyl]amino]phthalazin-1-yl]-5-(trifluoromethyl)phenol hydrochloride

tert-Butyl (3R)-3-[[4-[2-(methoxymethoxy)-4-(trifluoromethyl)phenyl]phthalazin-1-yl]amino]piperidine-1-carboxylate (55 mg, 0.10 mmol) was stirred in a solution of HCl (4 M in dioxane, 1 mL) at room temperature for 1 h. The organic volatiles were removed. The residue was triturated with diethyl ether and filtered to afford 2-[4-[[(3R)-3-piperidyl]amino]phthalazin-1-yl]-5-(trifluoromethyl)phenol (39 mg, 89% yield) as hydrochloride salt. MS m/z 389.7 [M+H]⁺; ¹H NMR (CD₃OD) δ: 8.73 (d, J=8.1 Hz, 1H), 8.31 (br t, J=7.5 Hz, 1H), 8.16 (t, J=7.7 Hz, 1H), 7.98 (br d, J=8.4 Hz, 1H), 7.71 (br d, J=8.1 Hz, 1H), 7.46 (br d, J=7.9 Hz, 1H), 7.39 (s, 1H), 4.62 (br d, J=4.4 Hz, 1H), 3.73-3.83 (m, 1H), 3.41-3.49 (m, 1H), 3.08-3.25 (m, 2H), 2.36 (br d, J=7.1 Hz, 1H), 2.20 (br dd, J=6.2, 3.9 Hz, 1H), 1.93-2.05 (m, 2H). 3 NHs and OH not observed.

### Step 3. 2-[4-[[(3R)-1-[2-[tert-Butyl(dimethyl)silyl]oxyethyl]-3-piperidyl]amino]phthalazin-1-yl]-5-(trifluoromethyl)phenol

A mixture of 2-[4-[[(3R)-3-piperidyl]amino]phthalazin-1-yl]-5-(trifluoromethyl)phenol (36 mg, 0.084 mmol, 1.0 eq.) hydrochloride, (tert-butyldimethylsilyloxy)acetaldehyde (18 mg, 0.092 mmol, 1.1 eq.) and sodium triacetoxyborohydride (55 mg, 3.0 eq.) in DIPEA (22 mg, 2.0 eq.) and DCM (0.6 mL) was stirred at rt for 15 mins. The reaction was diluted with DCM and NaHCO₃ (sat. aq.). The organic phase was washed with H₂O. The organic phase was dried over Na₂SO₄, filtered, and concentrated in vacuo. The crude material was purified by flash column chromatography on basic alumina eluting with 0-10% MeOH in DCM to provide 2-[4-[[(3R)-1-[2-[tert-butyl(dimethyl)silyl]oxyethyl]-3-piperidyl]amino]phthalazin-1-yl]-5-(trifluoromethyl)phenol (15 mg, 33% yield). MS m/z 546.8 [M+H]⁺.

### Step 4. 2-[4-[[(3R)-1-(2-Hydroxyethyl)-3-piperidyl]amino]phthalazin-1-yl]-5-(trifluoromethyl)phenol formic acid salt

2-[4-[[(3R)-1-[2-[tert-Butyl(dimethyl)silyl]oxyethyl]-3-piperidyl]amino]phthalazin-1-yl]-5-(trifluoromethyl)phenol (15 mg, 0.027 mmol) was stirred in a solution of HCl (4 M in dioxane, 1 mL) at room temperature for 1 h. The organic volatiles were removed. The residue was then purified on prep-HPLC with 5-50% ACN in water with 0.1% formic acid to provide 2-[4-[[(3R)-1-(2-hydroxyethyl)-3-piperidyl]amino]phthalazin-1-yl]-5-(trifluoromethyl)phenol (8 mg, 67% yield) as formic acid salt. MS m/z 433.3 [M+H]⁺. ¹H NMR (CD₃OD) δ: 8.43 (s, 1H, formic acid-H), 8.22 (br d, J=8.4 Hz, 1H), 7.80 (t, J=6.9 Hz, 1H), 7.72 (br t, J=7.6 Hz, 1H), 7.52 (br d, J=7.9 Hz, 1H), 7.43 (br d, J=7.8 Hz, 1H), 7.21 (br d, J=8.0 Hz, 1H), 7.15 (s, 1H), 4.54 (br s, 1H), 3.71 (br t, J=5.0 Hz, 2H), 3.35-3.50 (m, 1H), 2.93-3.06 (m, 1H), 2.72-2.89 (m, 2H), 2.52-2.72 (m, 2H), 1.97-2.08 (m, 1H), 1.84-1.96 (m, 1H), 1.66-1.84 (m, 2H). NH and 2 OH not observed.

### Example 15

### Preparation of Compounds I-65 and I-98

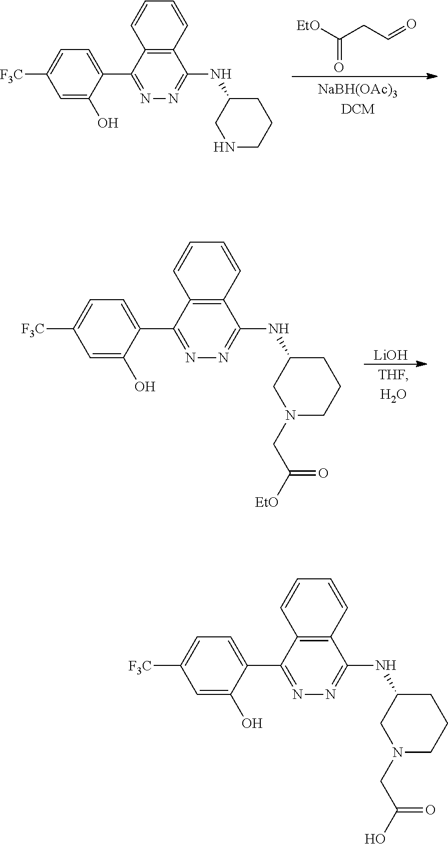

### Step 1. Ethyl (R)-2-(3-((4-(2-hydroxy-4-(trifluoromethyl)phenyl)phthalazin-1-yl)amino)piperidin-1-yl)acetate

A mixture of 2-[4-[[(3R)-3-piperidyl]amino]pyrido[3,4-d]pyridazin-1-yl]-5-(trifluoromethyl)phenol hydrochloride (Example 14, step 3, 50 mg, 0.11 mmol) and sodium perborate tetrahydrate (31 mg) in DIPEA (29 mg, 2.0 eq.) and DCM (0.5 mL) was stirred for 15 mins, followed by addition of ethyl 3-oxopropanoate (17 mg, 1.5 eq.) and NaBH(OAc)₃ (71 mg, 3.0 eq.). After 15 mins, the reaction was quenched by dilution with DCM and NaHCO₃ (sat.). The organic phase was washed with H₂O followed by brine. The organic phase was dried over Na₂SO₄, filtered, and concentrated in vacuo. The crude material was purified by flash column chromatography on silica gel eluting with 0-20% MeOH (with NH₄OH as modifier) in DCM to provide 2-[4-[[(3R)-1-cyclobutyl-3-piperidyl]amino]pyrido[3,4-d]pyridazin-1-yl]-5-(trifluoromethyl)phenol (45 mg, 88% yield). MS m/z 475.3 [M+H]⁺. ¹H NMR (CD₃OD) δ: 8.36 (d, J=8.3 Hz, 1H), 7.88-7.93 (m, 1H), 7.82 (t, J=7.6 Hz, 1H), 7.63 (d, J=8.1 Hz, 1H), 7.55 (d, J=7.9 Hz, 1H), 7.31 (br d, J=8.3 Hz, 1H), 7.26 (s, 1H), 4.62 (br s, 1H), 4.22 (q, J=7.1 Hz, 2H), 3.47-3.54 (m, 1H), 3.04 (br d, J=10.6 Hz, 1H), 2.58-2.76 (m, 3H), 1.79-2.00 (m, 3H), 1.74 (br d, J=6.1 Hz, 1H), 1.24-1.35 (t, J=6.9 Hz, 3H), 1.20 (t, J=6.9 Hz, 1H). NH and OH not observed.

### Step 2. (R)-2-(3-((4-(2-Hydroxy-4-(trifluoromethyl)phenyl)phthalazin-1-yl)amino)piperidin-1-yl)acetic acid

To a solution of ethyl 2-[(3R)-3-[[4-[2-hydroxy-4-(trifluoromethyl)phenyl]phthalazin-1-yl]amino]-1-piperidyl]acetate (40 mg, 0.084) in THE (2 mL) was added H₂O (0.5 mL), followed by LiOH (2 mg, 1.0 eq.). After stirring at rt for 2-3 hr, the reaction mixture was neutralized by citric acid to pH-7, then extracted with EtOAc. The organic volatiles were removed. The residue was then purified on prep-HPLC with 5-30% ACN in water with 0.1% formic acid to provide 2-[(3R)-3-[[4-[2-hydroxy-4-(trifluoromethyl)phenyl]phthalazin-1-yl]amino]-1-piperidyl]acetic acid (5 mg, 13% yield) as formic acid salt. MS m/z 447.2 [M+H]⁺. ¹H NMR (CD₃OD) δ: 8.33 (d, J=8.1 Hz, 1H), 7.89-7.97 (i, 9H), 7.82-7.89 (n, 1H), 7.64 (d, J=8.3 Hz, 1H), 7.54 (d, J=7.9 Hz, 1H), 7.29-7.36 (i, 1H), 7.24-7.29 (m, 1H), 4.77-4.87 (m, 1H), 3.78-3.96 (1H), 3.58-3.75 (m, 3H), 3.03-3.24 (i, 2H), 2.22-2.34 (i, 1H), 2.12-2.22 (i, 1H), 2.04 (br dd, J=11.3, 3.5 Hz, 1H), 1.89 (br d, J=10.3 Hz, 1H). NH and OH not observed.

The compounds below were prepared according to the procedure of Example 15 by substituting the appropriate starting materials, reagents and reaction conditions.

| Compound | Spectral Data |
|---|---|
| I-21 | MS m/z 390.1 [M + H]⁺, ¹H NMR (METHANOL-d4) δ: 8.52 (br d, J = 8.0 Hz, |
| | 1H), 7.90-8.09 (m, 2H), 7.72 (br d, J = 8.1 Hz, 1H), 7.56 (br d, J = 7.8 Hz, 1H), |
| | 7.24-7.43 (m, 2H), 5.88 (br s, 1H), 3.81 (br d, J = 11.6 Hz, 1H), 3.62 (br d, J = 12.9 |
| | Hz, 1H), 3.43 (br d, J = 12.1 Hz, 1H), 3.28-3.35 (m, 2H), 2.15-2.40 (m, 3H), 1.97 |
| | (br d, J = 13.9 Hz, 1H). |
| I-22 | MS m/z 404.2 [M + H]⁺; ¹H NMR (METHANOL-d4) δ: 8.45-8.54 (m, 1H), |
| | 8.38-8.43 (m, 1H), 7.87-8.04 (m, 2H), 7.70 (d, J = 8.0 Hz, 1H), 7.55 (d, J = 7.9 |
| | Hz, 1H), 7.27-7.41(m, 2H), 5.90 (br s, 1H), 3.84 (br s, 1H), 3.55 (br d, J = 12.5 |
| | Hz, 1H), 3.41-3.50 (m, 1H), 3.07-3.31 (m, 1H), 2.90 (s, 3H), 2.20-2.40 (m, 2H), |
| | 2.11 (br d, J = 10.9 Hz, 1H), 1.91-2.05 (m, 1H) |
| I-29 | MS m/z 404.2 [M + H]⁺, ¹H NMR (METHANOL-d4) δ: 8.37 (br d, J = 8.1 Hz, |
| | 1H), 8.23 (s, 2H), 7.90 (t, J = 7.0 Hz, 1H), 7.80-7.85 (m, 1H), 7.59 (d, J = 8.1 Hz, |
| | 1H), 7.43 (d, J = 7.9 Hz, 1H), 7.13-7.27 (m, 2H), 5.77 (br s, 1H), 3.68 (br s, 1H), |
| | 3.32-3.53 (m, 1H), 3.24-3.34 (m, 1H), 3.06 (br t, J = 10.1 Hz, 1H), 2.77 (s, 3H), |
| | 2.09-2.27 (m, 2H), 2.00 (br d, J = 12.9 Hz, 1H), 1.80-1.94 (m, 1H). |
| I-35 | MS m/z 390.7 [M + H]⁺, ¹H NMR (METHANOL-d4) δ: 9.73 (s, 1H), 8.90 (d, |
| | J = 5.5 Hz, 1H), 8.35 (s, 1H), 7.59 (d, J = 7.9 Hz, 1H), 7.52 (d, J = 5.6 Hz, 1H), |
| | 7.35 (br d, J = 7.9 Hz, 1H), 7.30 (s, 1H), 4.64-4.81 (m, 1H), 3.83 (br d, J = 10.1 |
| | Hz, 1H), 3.35-3.50 (m, 1H), 3.11 (br t, J = 11.0 Hz, 2H), 2.34 (br s, 1H), 2.19 (br |
| | s, 1H), 1.97 (br t, J = 9.6 Hz, 2H). |
| I-43 | MS m/z 418.2 [M + H]⁺, ¹H NMR (METHANOL-d4) δ: 9.73 (s, 1H), 8.91 (d, |
| | J = 5.6 Hz, 1H), 8.34 (br s, 2H), 7.59 (d, J = 7.9 Hz, 1H), 7.52 (d, J = 5.6 Hz, 1H), |
| | 7.36 (d, J = 7.9 Hz, 1H), 7.30 (s, 1H), 4.71-4.82 (m, 1H), 3.89-4.04 (m, 1H), |
| | 3.48-3.68 (m, 1H), 3.21-3.28 (m, 2H), 2.94-3.17 (m, 2H), 2.19-2.40 (m, 2H), |
| | 1.99 (br d, J = 10.6 Hz, 2H), 1.40 (t, J = 7.3 Hz, 3H) |
| I-45 | MS m/z 407.8 [M + H]⁺; ¹H NMR (MeOH-d4) d: 7.31 (d, J = 8 Hz, 1H), 7.22 (d, |
| | J = 8 Hz, 1H), 7.18 (s, 1H), 4.29-4.34 (m, 1H), 3.41-3.47 (m, 1H), 3.05-3.11 |
| | (m, 1H), 2.65-2.82 (m, 4H), 2.12-2.16 (m, 1H), 1.98 (t, J = 7 Hz, 2H), 1.90-1.95 |
| | (m, 1H), 1.63-1.80 (m, 2H), 1.06 (s, 6H). Three exchangeable protons are not |
| | seen. |
| I-46 | MS m/z 421.9 [M + H]⁺; ¹H NMR (MeOH-d4) d: 7.31 (d, J = 8 Hz, 1H), 7.22 (d, |
| | J = 8 Hz, 1H), 7.18 (s, 1H), 4.40-4.47 (m, 1H), 3.08-3.20 (m, 1H), 2.76-2.83 |
| | (m, 3H), 2.28-2.43 (m, 5H), 1.95-2.10 (m, 3H), 1.83-1.93 (m, 1H), 1.68-1.81 |
| | (m, 1H), 1.50-1.65 (m, 1H), 1.06 (s, 6H). Two exchangeable protons are not |
| | seen. |
| I-53 | MS m/z 405.2 [M + H]⁺; ¹H NMR (400 MHz, CD₃OD) δ 8.53 (s, 1H, formic |
| | acid), 8.15 (d, J = 8.3 Hz, 1H), 7.95 (t, J = 7.6 Hz, 1H), 7.86 (t, J = 7.6 Hz, 1H), |
| | 7.65 (d, J = 8.3 Hz, 1H), 7.54 (d, J = 7.9 Hz, 1H), 7.33 (br d, J = 8.0 Hz, 1H), 7.27 |
| | (s, 1H), 4.71-4.84 (m, 1H), 4.13-4.23 (m, 1H), 3.44-3.54 (m, 1H), 3.13-3.29 (m, |
| | 2H, overlapped with CD₃OD), 3.02 (br dd, J = 12.6, 5.9 Hz, 1H), 2.36-2.44 (m, |
| | 1H), 2.05 (dt, J = 13.4, 6.7 Hz, 1H). NH and OH not observed. |
| I-55 | MS m/z 434.5 [M + H]⁺; ¹H NMR (CD₃OD) δ: 9.74 (s, 1H), 8.85 (d, J = 5.8 Hz, |
| | 1H), 7.58 (d, J = 7.8 Hz, 1H), 7.49 (d, J = 5.8 Hz, 1H), 7.30 (d, J = 8.0 Hz, 1H), |
| | 7.26 (s, 1H), 4.59-4.68 (m, 1H), 3.68-3.81 (m, 2H), 3.10-3.24 (m, 1H), 2.52- |
| | 2.75 (m, 3H), 2.46 (br s, 2H), 1.99-2.13 (m, 1H), 1.90 (br dd, J = 8.4, 2.6 Hz, |
| | 1H), 1.72-1.83 (m, 2H), 1.16-1.21 (m, 1H), NH and OHs not observed. |
| I-63 | MS m/z 419.2 [M + H]⁺; ¹H NMR (400 MHz, CD₃OD) δ 8.34 (s, 1H, formic |
| | acid), 8.04 (br d, J = 7.9 Hz, 1H), 7.82 (br t, J = 7.5 Hz, 1H), 7.74 (br t, J = 7.6 Hz, |
| | 1H), 7.53 (br d, J = 8.1 Hz, 1H), 7.42 (br d, J = 7.8 Hz, 1H), 7.12-7.25 (m, 2H), |
| | 4.68 (br s, 1H), 4.11 (br s, 1H), 3.25 (br s, 1H), 2.94-3.19 (m, 2H), 2.53-2.72 |
| | (m, 3H), 2.20 (br d, J = 11.9 Hz, 1H), 1.93 (s, 1H), 1.65-1.89 (m, 1H). NH and |
| | OH not observed. |
| I-68 | MS m/z 389.1 [M + H]⁺; ¹H NMR (400 MHz, DMSO-d6) δ 9.68 (s, 1H), 8.41 (s, |
| | 1H), 8.34 (d, J = 8.3 Hz, 1H), 7.88 (t, J = 7.6 Hz, 1H), 7.83-7.68 (m, 2H), 7.48 |
| | (dd, J = 16.6, 8.0 Hz, 2H), 7.34-7.23 (m, 2H), 3.73-3.54 (m, 2H), 3.23-3.10 |
| | (m, 2H), 3.08-2.99 (m, 1H), 2.97-2.89 (m, 1H), 2.86-2.73 (m, 1H), 2.10- |
| | 1.93 (m, 1H), 1.81-1.58 (m, 1H). |
| I-83 | MS m/z 405.2 [M + H]⁺; ¹H NMR (400 MHz, CD₃OD) δ 8.40 (s, 1H, formic |
| | acid), 8.19 (br d, J = 8.1 Hz, 1H), 7.79-7.85 (m, 1H), 7.74 (br t, J = 7.5 Hz, 1H), |
| | 7.53 (br d, J = 8.1 Hz, 1H), 7.43 (br d, J = 7.9 Hz, 1H), 7.14-7.24 (m, 2H), 4.95 (br |
| | s, 1H), 4.27 (br s, 1H), 3.73 (br d, J = 10.6 Hz, 1H), 3.17 (br s, 1H), 3.05 (br d, |
| | J = 13.1 Hz, 1H), 2.80 (br t, J = 11.4 Hz, 1H), 2.26 (br d, J = 12.8 Hz, 1H), 1.97 (br |
| | t, J = 13.1 Hz, 1H). NH and OH not observed. |
| I-84 | MS m/z 419.2 [M + H]⁺; ¹H NMR (400 MHz, CD₃OD) δ 8.32 (d, J = 8.1 Hz, 1H), |
| | 7.91 (t, J = 7.2 Hz, 1H), 7.83 (t, J = 7.8 Hz, 1H), 7.63 (d, J = 8.3 Hz, 1H), 7.55 (d, |
| | J = 7.8 Hz, 1H), 7.32 (br d, J = 7.8 Hz, 1H), 7.26 (s, 1H), 4.80 (s, 1H, overlapped |
| | with CD3OD), 4.03-4.15 (m, 1H), 2.64-2.90 (m, 3H), 2.32-2.49 (m, 4H), 2.14- |
| | 2.29 (m, 1H), 1.76-1.91 (m, 1H). NH and OH not observed. |
| I-87 | MS m/z 403.2 [M + H]⁺; ¹H NMR (400 MHz, CD₃OD) δ 8.24 (d, J = 8.2 Hz, |
| | 1H), 7.89 (t, J = 7.7 Hz, 1H), 7.81 (t, J = 7.6 Hz, 1H), 7.62 (d, J = 8.2 Hz, 1H), |
| | 7.53 (d, J = 7.9 Hz, 1H), 7.30 (d, J = 7.9 Hz, 1H), 7.24 (d, J = 1.6 Hz, 1H), 3.76- |
| | 3.63 (m, 2H), 3.07-2.79 (m, 4H), 2.76-2.67 (m, 1H), 2.54 (s, 3H), 2.23- |
| | 2.11 (m, 1H), 1.86-1.73 (m, 1H). |
| I-101 | MS m/z 434.5 [M + H]⁺; ¹H NMR (METHANOL-d4) δ: 9.74 (s, 1H), 8.85 (d, |
| | J = 5.8 Hz, 1H), 7.58 (d, J = 7.8 Hz, 1H), 7.49 (d, J = 5.8 Hz, 1H), 7.30 (d, J = 8.0 |
| | Hz, 1H), 7.26 (s, 1H), 4.59-4.68 (m, 1H), 3.68-3.81 (m, 2H), 3.10-3.24 (m, 1H), |
| | 2.52-2.75 (m, 3H), 2.46 (m, 2H), 1.99-2.13 (m, 1H), 1.90 (br dd, J = 8.4, 2.6 |
| | Hz, 1H), 1.72-1.83 (m, 2H), 1.16-1.21 (m, 1H) |
| I-110 | MS m/z 390.5 [M + H]⁺; ¹H NMR (400 MHz, CD₃OD) δ 9.00-8.91 (m, 2H), |
| | 8.55 (s, 1H), 8.11 (d, J = 5.7 Hz, 1H), 7.61 (d, J = 7.8 Hz, 1H), 7.35 (d, J = 7.9 |
| | Hz, 1H), 7.28 (s, 1H), 3.87-3.75 (m, 2H), 3.52-3.39 (m, 3H), 3.21-3.14 (m, |
| | 1H), 3.03-2.91 (m, 1H), 2.32-2.21 (m, 1H), 1.99-1.86 (m, 1H). |
| I-111 | MS m/z 390.2 [M + H]⁺; ¹H NMR (400 MHz, CD₃OD) δ 9.62 (d, J = 1.1 Hz, |
| | 1H), 8.88 (d, J = 5.7 Hz, 1H), 8.45 (s, 1H), 7.57 (d, J = 7.9 Hz, 1H), 7.50 (d, J = |
| | 5.7 Hz, 1H), 7.33 (d, J = 8.0 Hz, 1H), 7.29-7.24 (m, 1H), 3.90-3.77 (m, 2H), |
| | 3.55-3.41 (m, 2H), 3.37-3.30 (m, 1H), 3.24-3.16 (m, 1H), 3.05-2.93 (m, |
| | 1H), 2.35-2.20 (m, 1H), 2.02-1.88 (m, 1H). |
| I-112 | MS m/z 404.3 [M + H]⁺; ¹H NMR (400 MHz, CD₃OD) δ 9.66-9.57 (m, 1H), |
| | 8.86 (d, J = 5.7 Hz, 1H), 8.55 (s, 1H), 7.57 (d, J = 7.9 Hz, 1H), 7.49 (d, J = 5.8 |
| | Hz, 1H), 7.32 (d, J = 7.9 Hz, 1H), 7.27 (s, 1H), 3.82-3.73 (m, 2H), 3.25-3.18 |
| | (m, 1H), 3.15-2.90 (m, 4H), 2.69 (s, 3H), 2.33-2.19 (m, 1H), 1.96-1.81 (m, |
| | 1H). |
| I-151 | MS m/z 421.2 [M + H]⁺; ¹H NMR (METHANOL-d4) δ: 8.30 (d, J = 8.3 Hz, 1H), |
| | 7.90 (t, J = 7.2 Hz, 1H), 7.80-7.85 (m, 1H), 7.62 (d, J = 8.1 Hz, 1H), 7.55 (d, J = 7.8 |
| | Hz, 1H), 7.31 (d, J = 8.0 Hz, 1H), 7.26 (s, 1H), 5.06 (br s, 1H), 4.91-4.97 (m, |
| | 1H), 3.23-3.29 (m, 1H), 2.94-3.06 (m, 1H), 2.35-2.52 (m, 5H), 2.22-2.30 (m, |
| | 1H), 1.84-2.05 (m, 1H). OH and NH not observed. |
| I-164 | MS m/z 421.2 [M + H]⁺; ¹H NMR (METHANOL-d4) δ: 8.28-8.32 (m, J = 8.3 Hz, |
| | 1H), 7.88-7.95 (m, 1H), 7.78-7.86 (m, 1H), 7.61-7.67 (m, J = 8.1 Hz, 1H), 7.55 |
| | (d, J = 7.8 Hz, 1H), 7.29-7.35 (m, 1H), 7.23-7.29 (m, 1H), 5.05-5.12 (m, 1H), |
| | 4.93-5.00 (m, 1H), 3.23-3.29 (m, 1H), 2.94-3.06 (m, 1H), 2.35-2.52 (m, 5H), |
| | 2.22-2.30 (m, 1H), 1.84-2.05 (m, 1H). OH and NH not observed. |
| I-235 | MS m/z 468.0 [M + H]⁺; ¹H NMR (400 MHz, DMSO-d6) δ 8.98 (d, J = 5.8 Hz, |
| | 1H), 8.81 (s, 1H), 8.35 (d, J = 5.88 Hz, 1H), 8.22 (s, 1H, formic acid), 7.87- |
| | 7.80 (m, 2H), 7.74 (s, 1H), 7.66 (s, 1H), 7.38 (t, J = 73.67 Hz, 1 H), 4.49- |
| | 4.34 (m, 1H), 3.22-3.13 (m, 1H), 2.89-2.78 (m, 1H), 2.40 (q, J = 7.3 Hz, |
| | 2H), 2.08-1.88 (m, 3H), 1.81-1.67 (m, 1H), 1.65-1.42 (m, 2H), 1.02 (t, J = |
| | 7.1 Hz, 3H) |
| I-278 | MS m/z 422.0 [M + H]⁺; ¹H NMR (400 MHz, DMSO-d6) δ 9.78 (s, 1 H), 8.85 |
| | (d, J = 5.63 Hz, 1 H), 8.23 (s, 1 H, formic acid), 7.69 (d, J = 8.63 Hz, 1 H), 7.56 |
| | (d, J = 7.88 Hz, 1 H), 7.35-7.27 (m, 3 H), 5.02 (d, J = 45.53 Hz, 1 H), 4.84- |
| | 4.72 (m, 1 H), 3.11 (dd, J = 11.01, 2.00 Hz, 1 H), 2.99-2.87 (m, 1 H), 2.34- |
| | 2.20 (m, 4 H), 2.20-2.11 (m, 1 H), 2.09-1.98 (m, 1 H), 1.94-1.64 (m, 1 H). |
| | 1H not observed (OH). |
| I-279 | MS m/z 472.2 [M − H]−; ¹H NMR (400 MHz, METHANOL-d4) δ = 9.70 (s, 1H), |
| | 8.85 (d, J = 5.8 Hz, 1H), 7.59 (d, J = 7.9 Hz, 1H), 7.48 (d, J = 5.6 Hz, 1H), 7.32 |
| | (d, J = 8.0 Hz, 1H), 7.30-7.26 (m, 1H), 4.68-4.57 (m, 1H), 4.01 (br dd, J = |
| | 3.1, 11.3 Hz, 2H), 3.49-3.35 (m, 3H), 2.94 (br d, J = 11.0 Hz, 1H), 2.77-2.57 |
| | (m, 1H), 2.57-2.36 (m, 2H), 2.21-2.08 (m, 1H), 1.96-1.84 (m, 3H), 1.84- |
| | 1.56 (m, 4H); NH and OH wasn't observed |
| I-280 | MS m/z [M + H]⁺; 422.0; ¹H NMR (400 MHz, DMSO-d6) δ 9.78 (s, 1H), 8.85 |
| | (d, J = 5.75, 1H), 8.20 (s, 1H, formic acid), 7.70 (d, J = 7.6 Hz, 1H), 7.56 (d, |
| | J = 7.6 Hz, 1H), 7.35-7.25 (m, 3H), 5.02 (d, J = 46.4 Hz, 1H), 4.85-4.72 (m, |
| | 1H), 3.15-3.06 (m, 1H), 3.01-2.89 (m, 1H), 2.34-2.20 (m, 4H), 2.20-2.11 |
| | (m, 1H), 2.10-1.96 (m, 1H), 1.94-1.64 (m, 1H). 1H not observed (OH) |
| I-311 | MS m/z 440.5 [M + H]⁺; ¹H NMR (400 MHz, METHANOL-d4) δ 9.84-9.68 |
| | (m, 1H), 8.90 (d, J = 5.6 Hz, 1H), 8.55 (s, 1H), 7.79 (s, 2H), 7.71 (s, 1H), 7.40 |
| | (d, J = 5.6 Hz, 1H), 7.15-6.65 (m, 1H), 4.79-4.48 (m, 2H), 3.71 (br d, J = |
| | 12.3 Hz, 1H), 3.11-2.92 (m, 2H), 2.29 (br s, 1H), 2.11 (br s, 1H), 2.00-1.78 |
| | (m, 2H). NH not observed |
| I-312 | MS m/z 494.4 [M + H]⁺; ¹H NMR (400 MHz, METHANOL-d4) δ 9.85-9.65 |
| | (m, 1H), 8.97-8.77 (m, 1H), 7.78 (s, 2H), 7.69 (s, 1H), 7.36 (d, J = 5.6 Hz, |
| | 1H), 7.12-6.66 (m, 1H), 4.73-4.56 (m, 1H), 3.25-3.12 (m, 1H), 2.99-2.87 |
| | (m, 1H), 2.83-2.71 (m, 1H), 2.29-2.07 (m, 5H), 2.06-1.94 (m, 2H), 1.93- |
| | 1.84 (m, 1H), 1.83-1.65 (m, 4H). NH not observed |

### Example 16

### Preparation of Compound I-124

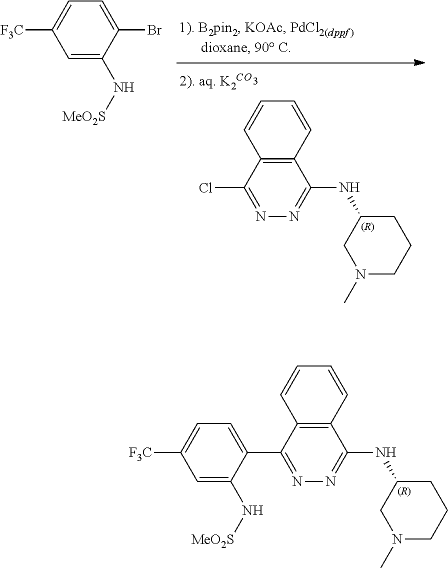

A mixture of N-[2-bromo-5-(trifluoromethyl)phenyl]-methanesulfonamide (prepared according to EP1837329, 2007, A1, 0.058 g, 0.18 mmol), B₂pin₂ (0.058 g, 0.23 mmol), KOAc (0.054 g, 0.055 mmol) and Pd(dppf)Cl₂ (0.005 g, 0.009 mmol) was evacuated and backfilled with Ar (3×) before being dissolved in dioxane (0.9 mL). The reaction was heated to 90° C. for 18 h, then cooled and to which was added 4-chloro-N-[(3R)-1-methyl-3-piperidyl]phthalazin-1-amine (Intermediate 19a, 0.048 g, 0.17 mmol), PdCl₂(dppf) (0.0055 g, 0.007 mmol), and K₂CO₃ (2 M, 0.28 mL, 0.55 mmol). The mixture was sparged with Ar for 5 min. The reaction was then heated to 95° C. for 3 h. The mixture was diluted with EtOAc and filtered through Celite. The filtrate was washed with brine and concentrated. Purification by chromatography on SiO₂ (MeOH:DCM, 0 to 10%) then reverse phase chromatography (0.1% formic acid in MeCN:0.1% formic acid in H₂O, 5 to 100%) gave a white solid (0.018 g, 19%). MS m/z 480.2 [M+H]⁺; ¹H NMR (400 MHz, CD₃OD) δ: 8.46 (s, 1H, formic acid), 8.36 (d, J=8.50 Hz, 1H), 8.04-7.99 (m, 1H), 7.99-7.91 (m, 1H), 7.91-7.82 (m, 1H), 7.74-7.59 (m, 3H), 4.76-4.58 (m, 1H), 2.90 (s, 3H), 2.79 (s, 3H), 2.59-2.43 (m, 1H), 2.31-2.06 (m, 2H), 2.02-1.77 (m, 2H), 1.74 -1.63 (m, 1H), 0.84-0.66 (m, 2H). 2 Hs not observed (2 NH).

The compounds below were prepared according to the procedure of Example 16 by substituting the appropriate starting materials, reagents and reaction conditions.

| Compound | Spectral Data |
|---|---|
| I-126 | MS m/z 444.2 [M + H]⁺; ¹H NMR (400 MHz, CD₃OD) δ: 8.43-8.32 (m, 2 H), |
| | 8.22 (s, 1 H, formic acid), 7.95 (t, J = 7.63 Hz, 1 H), 7.85 (t, J = 8.63 Hz, 1 H), |
| | 7.74-7.65 (m, 2 H), 7.55 (d, J = 8.25 Hz, 1 H), 4.76-4.65 (m, 1 H), 3.89- |
| | 3.76 (m, 1 H), 3.50-3.39 (m, 1 H), 3.14-2.98 (m, 2 H), 2.89 (s, 3 H), 2.30- |
| | 2.14 (m, 2 H), 2.05-1.86 (m, 2 H), 1.79 (s, 3 H). 2Hs not observed (2 NH) |
| I-204 | MS m/z 455.9 [M + H]⁺; ¹H NMR (CD₃OD) δ: 9.78 (d, J = 0.8 Hz, 1H), 8.92 (d, |
| | J = 5.8 Hz, 1H), 8.31 (d, J = 7.5 Hz, 1H), 7.91 (d, J = 7.3 Hz, 1H), 7.66 (t, J = 71.5 |
| | Hz, 1H), 7.50 (dd, J = 5.8, 0.8 Hz, 1H), 4.68-4.76 (m, 1H), 3.43-3.54 (m, 1H), |
| | 3.01-3.09 (m, 1H), 2.64-2.77 (m, 2H), 2.63 (s, 3H), 2.15-2.27 (m, 1H), 2.01- |
| | 2.12 (m, 1H), 1.77-1.93 (m, 2H); 1H not observed (NH). |

### Example 17

### Preparation of Compound I-214

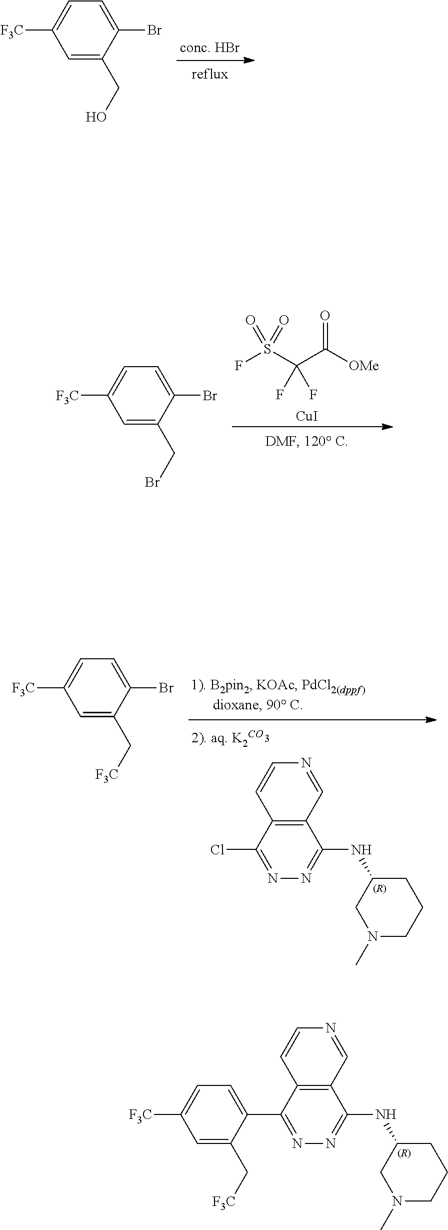

### Step 1: 1-Bromo-2-(bromomethyl)-4-trifluoromethyl)benzene

[2-Bromo-5-(trifluoromethyl)phenyl]methanol (1.70 g, 6.67 mmol) in conc. HBr (8 mL) was heated to reflux for 2 h. The reaction was cooled to rt and extracted with DCM. The combined organic extracts were washed with sat. NaHCO₃, brine, dried (Na₂SO₄), filtered and concentrated to give a pale yellow oil. Purification by chromatography on SiO₂ (EtOAc:hexanes, 0 to 10%) gave a colorless oil (1.12 g, 53%). ¹H NMR (400 MHz, CDCl₃) δ 7.77-7.70 (m, 2H), 7.43 (dd, J=8.38 Hz, 2.00 Hz, 1H), 4.62 (s, 2H).

### Step 2. 1-Bromo-2-(2,2,2-trifluoroethyl)-4-(trifluoromethyl)benzene

To a solution of 1-bromo-2-(bromomethyl)-4-(trifluoromethyl)benzene (1.20 g, 3.77 mmol) in DMF (8.4 mL) was added CuI (1.80 g, 9.44 mmol), and the solution was sparged with Ar. To this solution was added difluoro-fluorosulfonyl-acetic acid methyl ester (1.20 mL, 9.44 mmol), and the resulting reaction mixture was heated at 120° C. for 4 h The reaction mixture was cooled to 0° C., diluted with EtOAc (60 ml) and stirred for 10 minutes at 0° C. A solution of ammonium hydroxide (conc., 6 mL) was added dropwise and the mixture was stirred as it warmed to rt. EtOAc (100 mL) and water (50 mL) were added and the layers were separated. The aqueous layer was further extracted with EtOAc. The combined organic layers were washed with water and brine, dried (Na₂SO₄), and concentrated. Purification by chromatography on SiO₂ (EtOAc:hexanes, 0 to 10%) gave a yellow oil (0.215 g, 19%).

### Step 3: (R)—N-(1-Methylpiperidin-3-yl)-1-(2-(2,2,2-trifluoroethyl)-4-(trifluoromethyl)-phenyl)pyrido[3,4-d]pyridazin-4-amine formic acid salt

The titled compound was prepared according to the procedure of 16. MS m/z 470.5 [M+H]⁺; ¹H NMR (400 MHz, DMSO-d₆) δ 9.84 (s, 1H), 8.86 (d, J=5.6 Hz, 1H), 8.18 (s, 1H, formic acid), 8.03 (s, 1H), 7.92 (d, J=8.1 Hz, 1H), 7.78 (d, J=7.6 Hz, 1H), 7.68 (d, J=9.26 Hz, 1H), 7.22 (d, J=5.8 Hz, 1H), 4.56-4.43 (m, 1H), 4.11-3.59 (m, 2H), 3.16-3.05 (m, 1H), 2.78-2.69 (m, 1H), 2.23 (s, 3H), 2.08-1.85 (m, 3H), 1.83-1.71 (m, 1H), 1.69-1.55 (m, 1H), 1.55-1.39 (m, 1H)

### Example 18

### (Preparation of Compound I-237

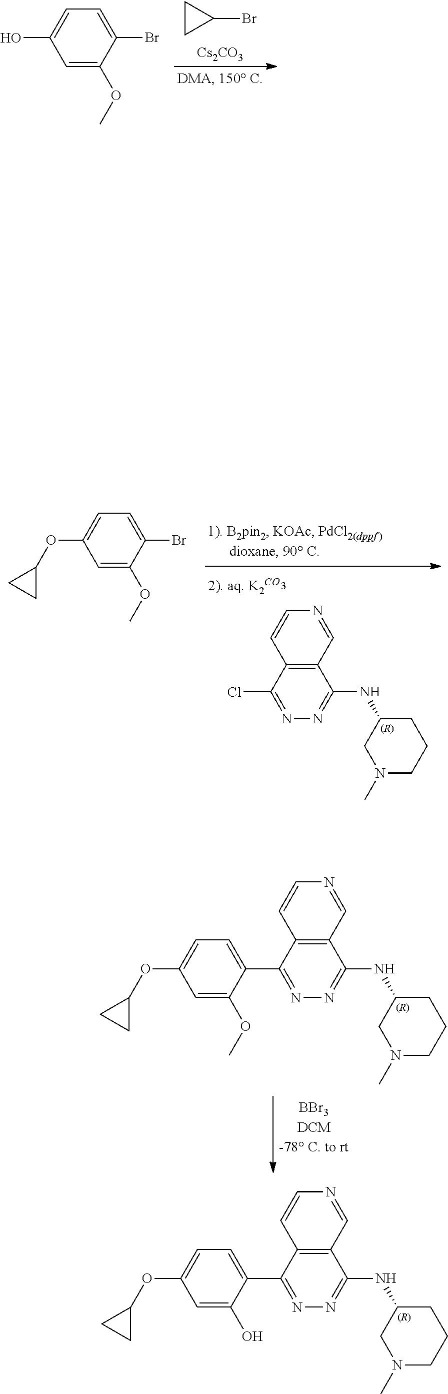

### Step 1: 1-Bromo-4-(cyclopropoxy)-2-methoxy-benzene

A solution of 4-bromo-3-methoxy-phenol (0.750 g, 53.69 mmol) in DMA (10.7 ml) was added Cs₂CO₃ (3.01 g, 9.23 mmol). The mixture was stirred for 5 min before bromocyclopropane (1.48 mL, 18.5 mmol) was added. The mixture was heated to 150° C. for 24 h. The reaction was diluted with H₂O and extracted with EtOAc. The combined organic extracts were washed with brine, dried (MgSO₄), filtered and concentrated. Purification by chromatography on SiO₂ (EtOAc:hexanes, 0 to 20%) gave a light-yellow oil (0.591 g, 66%). ¹H NMR (400 MHz, CDCl₃) δ 7.42 (d, J=8.63 Hz, 2H), 6.67-6.60 (m, 2H), 6.60-6.56 (m, 1H), 3.87 (s, 1H), 3.77-3.70 (m, 1H), 0.82-0.76 (m, 4H).

### Step 2. 5-(Cyclopropoxy)-2-[4-[[(3R)-1-methyl-3-piperidyl]amino]pyrido[3,4-d]pyridazin-1-yl]phenol

5-(Cyclopropoxy)-2-[4-[[(3R)-1-methyl-3-piperidyl]amino]pyrido[3,4-d]pyridazin-1-yl]phenol was prepared according to the procedure of Example 16 (0.089 g, 54%). MS m/z 406.6 [M+H]⁺.

### Step 3: (R)-5-Cyclopropoxy-2-(4-((1-methylpiperidin-3-yl)amino)pyrido[3,4-d]pyridazin-1-yl)phenol formic acid salt

A solution 5-(cyclopropoxy)-2-[4-[[(3R)-1-methyl-3-piperidyl]amino]pyrido[3,4-d]pyridazin-1-yl]phenol (0.089 g, 0.22 mmol) in DCM (1.1 mL) was cooled to −78° C. and BBr₃ (1.0 M in DCM, 1.8 ml, 1.8 mmol) was added. The reaction was stirred at −78° C. for 30 min and let warm to rt. The reaction was then stirred for 1.5 h before quenching by the addition of MeOH (0.5 mL). The mixture was diluted with DCM (20 mL), stirred 10 min. Sat. NaHCO₃ (1.0 mL) was added and vigorously stirred for 15 min. The layers were separated, and the aqueous phase was extracted with DCM. The combined organic extracts were dried (Na₂SO₄), filtered and concentrated. Purification by reverse phase chromatography (0.1% formic acid in MeCN:0.1% formic acid in H₂O, 5 to 100%) gave a tan solid (0.009 g, 10%). MS m/z 392.5 [M+H]⁺; H NMR (400 MHz, DMSO-d6) δ 9.71 (s, 1H), 8.88 (d, J=6.75 Hz, 1H), 8.31 (s, 1H, formic acid), 7.81 (d, J=7.88 Hz, 1H), 7.38 (d, J=6.00 Hz, 1H), 7.25 (d, J=8.51 Hz, 1H), 6.72 (d, J=3.00 Hz, 1H), 6.67 (dd, J=8.3, 2.4 Hz, 1H), 5.09-4.96 (m, 1H), 3.88-3.77 (m, 2H), 3.68-3.41 (m, 2H), 3.26-3.16 (m, 3H), 2.27-2.11 (m, 3H), 2.11-1.96 (m, 1H), 1.74-1.57 (m, 1H), 0.88-0.77 (m, 2H), 0.75-0.68 (m, 2H). 1H not observed (OH).

### Example 19

### Preparation of Compound I-274

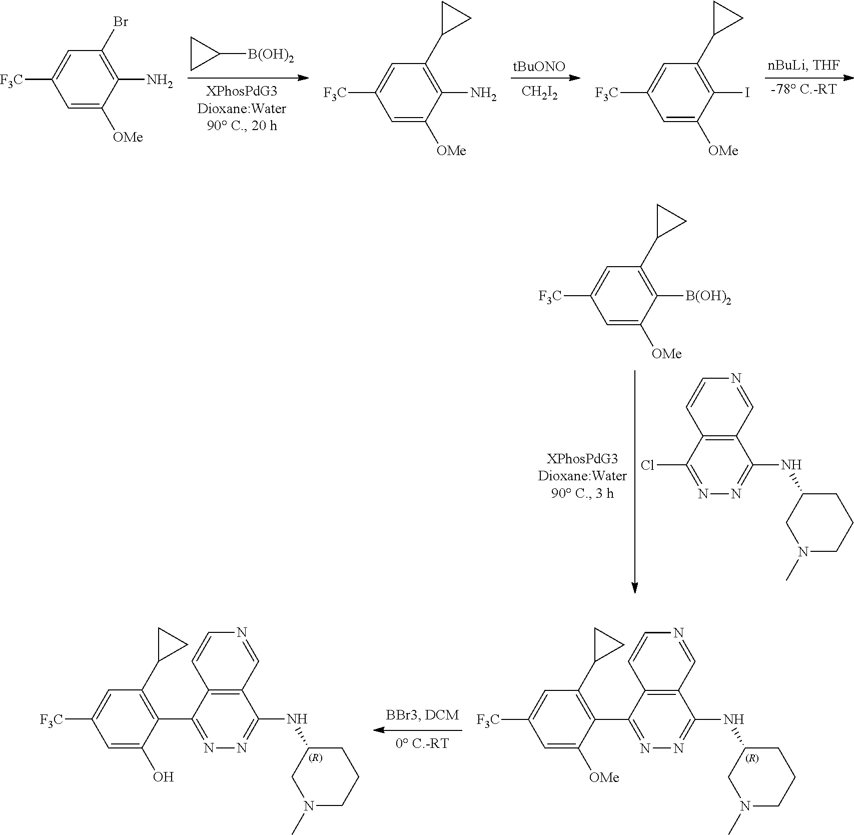

### Step 1: 2-Cyclopropyl-6-methoxy-4-(trifluoromethyl) aniline

To a vial was added 2-bromo-6-methoxy-4-(trifluoromethyl) aniline (5.1 g, 19 mmol), cyclopropylboronic acid (1.5 eq., 2.4 g, 28 mmol), (2-dicyclohexylphosphino-2′,4′,6′-triisopropyl-1,1′-biphenyl)[2-(2′-amino-1,1′-biphenyl)]palladium(II) methanesulfonate (0.1 eq., 1.6 g, 1.9 mmol), and potassium carbonate (3 eq., 7.8 g, 57 mmol) under Ar. 1,4-Dioxane:water (4:1) (100 mass %) was added and the resulting mixture was warmed to 90° C., and stirred for 20 h. The crude material was partitioned between EtOAc and brine. The brine was extracted with EtOAc (2×). The organic phase was concentrated and purified by column chromatography, eluting with 0-100% DCM/MeOH to afford 2-cyclopropyl-6-methoxy-4-(trifluoromethyl) aniline (2.6 g, 60% Yield), MS m/z 231.9 [M+H]⁺

### Step 2. 1-Cyclopropyl-2-iodo-3-methoxy-5-(trifluoromethyl)benzene

To a vial was added 2-cyclopropyl-6-methoxy-4-(trifluoromethyl) aniline (1.0 g, 4.3 mmol), acetonitrile (0.2 M, 22 mL), and tert-butyl nitrite (3 eq., 1.5 mL, 13 mmol). Next, at 0° C. diiodomethane (1.5 eq., 0.52 mL, 6.5 mmol) was added dropwise and the resulting mixture was warmed to 60° C., and stirred for 6 h. The crude material was partitioned between EtOAc and brine and the brine was extracted with EtOAc (2×). The organic phase was concentrated and purified by column chromatography, eluting with 0-100% Hexane/EtOAc to afford 1-cyclopropyl-2-iodo-3-methoxy-5-(trifluoromethyl)benzene (1-cyclopropyl-2-iodo-3-methoxy-5-(trifluoromethyl)benzene (390 mg, 26% Yield). ¹H NMR (CHLOROFORM-d) δ: 7.48-7.57 (s, 1H), 6.85 (s, 1H), 3.96-4.01 (s, 3H), 2.16 (m, 1H), 1.11 (m, 2H), 0.72 (m, 2H).

### Step 3: [2-Cyclopropyl-6-methoxy-4-(trifluoromethyl)phenyl]boronic acid

To a solution of 1-cyclopropyl-2-iodo-3-methoxy-5-(trifluoromethyl)benzene (390 mg, 1.14 mmol) in THE (0.2 M, 5.7 ml) was added n-BuLi (2.5 M in hexane, 1.5 eq. 0.68 ml) dropwise. The resulting mixture was stirred at −78° C. for 30 minutes, after which triisopropyl borate (2 eq., 0.52 ml) was added. The mixture was stirred at −78° C. for another 10 minutes, then allowed to warm to RT and stirred for 2 h. The mixture was partitioned between EtOAc and brine. The aqueous phase was extracted with EtOAc (×3). The combined organic phase was concentrated which was used for the next step without further purification.

### Step 4: 1-[2-Cyclopropyl-6-methoxy-4-(trifluoromethyl)phenyl]-N-[(3R)-1-methyl-3-piperidyl]pyrido[3,4-d]pyridazin-4-amine

To a mixture of 1-chloro-N-[(3R)-1-methyl-3-piperidyl]pyrido[3,4-d]pyridazin-4-amine (100 mg, 0.36 mmol), [2-cyclopropyl-6-methoxy-4-(trifluoromethyl)phenyl]boronic acid (1 eq., 0.36 mmol), (2-Dicyclohexylphosphino-2′,4′,6′-triisopropyl-1,1′-biphenyl)[2-(2′-amino-1,1′-biphenyl)]palladium(II) methanesulfonate (0.1 eq., 0.036 mmol) and potassium carbonate (3 eq., 1.08 mmol) was added 1,4-dioxane: water (4:1) (0.25 M, 1.4 ml) under Ar. The resulting mixture was warmed to 90° C. and stirred for 3 h. The mixture was then partitioned between EtOAc and brine. The aqueous phase was extracted with EtOAc (×2). The combined organic phase was concentrated and the residue was purified by column chromatography, eluting with 0-50% DCM/MeOH to afford 1-[2-cyclopropyl-6-methoxy-4-(trifluoromethyl)phenyl]-N-[(3R)-1-methyl-3-piperidyl]pyrido[3,4-d]pyridazin-4-amine (144 mg, 0.31 mmol, 87% Yield) MS m/z 458.9 [M+H]⁺.

### Step 5: 3-Cyclopropyl-2-[4-[[(3R)-1-methyl-3-piperidyl]amino]pyrido[3,4-d]pyridazin-1-yl]-5-(trifluoromethyl)phenol and 3-cyclopropyl-2-[4-[[(3R)-1-methyl-3-piperidyl](14N)amino]pyrido[3,4-d]pyridazin-1-yl]-5-(trifluoromethyl)phenol

To a solution of 1-[2-cyclopropyl-6-methoxy-4-(trifluoromethyl)phenyl]-N-[(3R)-1-methyl-3-piperidyl]pyrido[3,4-d]pyridazin-4-amine (144 mg, 0.31 mmol) in dichloromethane (0.1 M, 3.1 ml) at 0° C. was added BBr₃ (10 eq., 0.29 ml) dropwise. The resulting mixture was warmed to RT and stirred for 2 h, then quenched by careful addition of potassium carbonate and methanol in ice bath. The mixture was filtered to get rid of potassium carbonate and concentrated on vacuum. The residue was then purified on prep-HPLC with 5-50% ACN in water with 0.1% formic acid to provide 3-cyclopropyl-2-[4-[[(3R)-1-methyl-3-piperidyl]amino]pyrido[3,4-d]pyridazin-1-yl]-5-(trifluoromethyl)phenol (6 mg, 4.2% yield) as formic acid salt. 3-Cyclopropyl-2-[4-[[(3R)-1-methyl-3-piperidyl]amino]pyrido[3,4-d]pyridazin-1-yl]-5-(trifluoromethyl)phenol formic acid salt: MS m/z 444.3 [M+H]⁺, ¹H NMR (400 MHz MeOD-d4) δ: 9.63 (s, 1H), 8.75 (d, J=5.5 Hz, 1H), 8.42 (s, 1H, formic acid), 7.20 (d, J=5.4 Hz, 1H), 6.96 (s, 1H), 6.77 (s, 1H), 4.50-4.66 (m, 1H), 3.30-3.41 (m, 1H), 2.99-3.17 (m, 1H), 2.79-2.97 (m, 1H), 2.53-2.61 (m, 1H), 2.49 (s, 3H), 2.00-2.16 (m, 1H), 1.84-1.97 (m, 1H), 1.58-1.82 (m, 2H), 1.39-1.53 (m, 1H), 0.71 (br d, J=7.5 Hz, 2H), 0.39-0.50 (m, 1H), 0.26-0.39 (m, 1H). NH and OH not observed.

### Example 20

### Preparation of Compounds I-11 and I-62

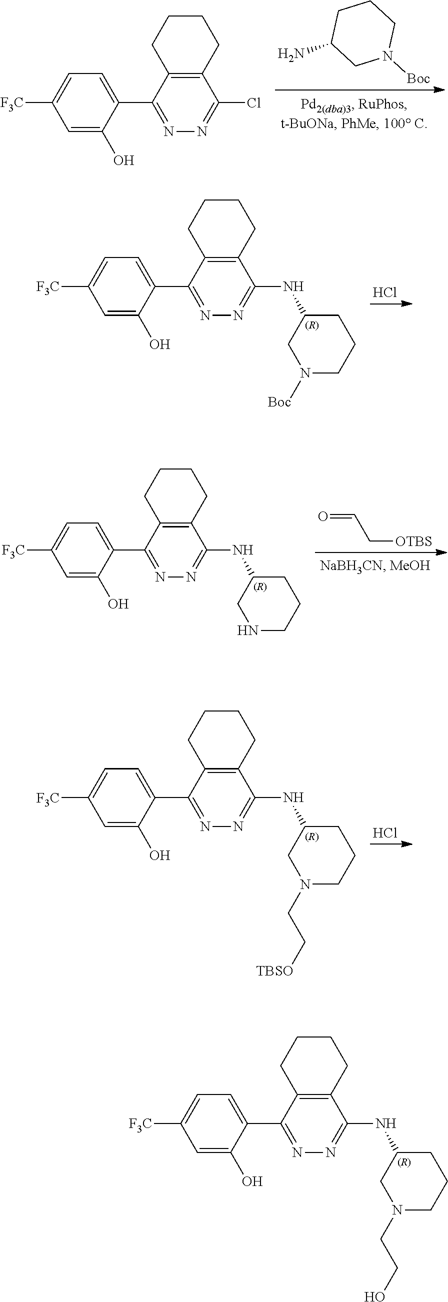

### Step 1. tert-Butyl (3R)-3-[[4-[2-hydroxy-4-(trifluoromethyl)phenyl]-5,6,7,8-tetrahydrophthalazin-1-yl]amino]piperidine-1-carboxylate

To a solution of tert-butyl (3R)-3-aminopiperidine-1-carboxylate (Intermediate 12, 366 mg, 1.83 mmol), and 2-(4-chloro-5,6,7,8-tetrahydrophthalazin-1-yl)-5-(trifluoromethyl)phenol (600 mg, 1.83 mmol) in toluene (6 mL) was added sodium tert-butoxide (360 mg, 3.74 mmol), RuPhos (210 mg, 0.44 mmol) and tris-(dibenzylideneacetone)-dipalladium(0) (210 mg, 0.22 mmol). The reaction was stirred at 100° C. for 2 h under N₂. The reaction mixture was evaporated in vacuo. The crude residue was purified over silica gel using 20%-30% EA/PE to give tert-butyl (3R)-3-[[4-[2-hydroxy-4-(trifluoromethyl)phenyl]-5,6,7,8-tetrahydrophthalazin-1-yl]amino]piperidine-1-carboxylate (280 mg, 31% Yield) as a yellow solid. MS m/z 493.2 [M+H]⁺.

### Step 2. (R)-2-(4-(Piperidin-3-ylamino)-5,6,7,8-tetrahydrophthalazin-1-yl)-5-(trifluoromethyl)phenol

To a solution of tert-butyl (3R)-3-[[4-[2-hydroxy-4-(trifluoromethyl)phenyl]-5,6,7,8-tetrahydrophthalazin-1-yl]amino]piperidine-1-carboxylate (330 mg, 0.67 mmol) was added hydrochloric acid in dioxane (10 mL, 40 mmol, 4 mol/L). The mixture was stirred at rt for 2.5 h, monitored by LCMS. The mixture was concentrated under vacuum to provide 2-[4-[[(3R)-3-piperidyl]amino]-5,6,7,8-tetrahydrophthalazin-1-yl]-5-(trifluoromethyl)phenol (270 mg, 100% Yield) as a pale powder. The crude product was applied to the next step without further purification. MS m/z 393.2 [M+H]⁺.

The crude product can be purified by Prep-HPLC eluting with 5-50% ACN in water with 0.1% formic acid. ¹H NMR (MeOD-d4) δ:7.34 (d, J=7.7 Hz, 1H), 7.19 (d, J=7.9 Hz, 1H), 7.15 (s, 1H), 4.31-4.23 (m, 1H), 3.38-3.33 (m, 1H), 3.05-2.98 (m, 1H), 2.73-2.60 (m, 2H), 2.50-2.42 (m, 4H), 2.11 (s, 1H), 1.94-1.80 (m, 3H), 1.76-1.62 (m, 4H). NH and OH not observed.

### Step 3. 2-[4-[[(3R)-1-[2-[tert-butyl(dimethyl)silyl]oxyethyl]-3-piperidyl]amino]-5,6,7,8-tetrahydrophthalazin-1-yl]-5-(trifluoromethyl)phenol

To a solution of 2-[4-[[(3R)-3-piperidyl]amino]-5,6,7,8-tetrahydrophthalazin-1-yl]-5-(trifluoromethyl)phenol (270 mg, 0.69 mmol) in methanol (5 mL) was added sodium acetate (60 mg, 0.73 mmol) and 2-[tert-butyl(dimethyl)silyl]oxyacetaldehyde (135 mg, 0.77 mmol), followed by sodium cyanoborohydride (130 mg, 2.069 mmol). The mixture was stirred at rt for 16 h, then concentrated under vacuum. The residue was purified by column chromatography (0-5% MeOH in DCM) to afford 2-[4-[[(3R)-1-[2-[tert-butyl(dimethyl)silyl]oxyethyl]-3-piperidyl]amino]-5,6,7,8-tetrahydrophthalazin-1-yl]-5-(trifluoromethyl)phenol (130 mg, 0.24 mmol, 34% Yield). 551.3 [M+H]⁺.

### Step 4. 2-[4-[[(3R)-1-(2-hydroxyethyl)-3-piperidyl]amino]-5,6,7,8-tetrahydrophthalazin-1-yl]-5-(trifluoromethyl)phenol

To a solution of 2-[4-[[(3R)-1-[2-[tert-butyl(dimethyl)silyl]oxyethyl]-3-piperidyl]amino]-5,6,7,8-tetrahydrophthalazin-1-yl]-5-(trifluoromethyl)phenol (4, 130 mg, 0.24 mmol) in DCM (2 mL) was added TFA (2 mL). This mixture was stirred at rt for 2 h then concentrated under vacuum. Sat.NaHCO₃ was added to adjust the pH=7 and the mixture was extracted with EA.

The organic layers was washed with water and brine, then dried and concentrated under vacuum to give a residue which was purified by column chromatography (0-5% MeOH in DCM) to afford 2-[4-[[(3R)-1-(2-hydroxyethyl)-3-piperidyl]amino]-5,6,7,8-tetrahydrophthalazin-1-yl]-5-(trifluoromethyl)phenol (40 mg, 39% Yield). MS m/z 437.0 [M+H]⁺. ¹H NMR (400 MHz, MeOD-d4) δ 7.35 (d, J=8.0 Hz, 1H), 7.21 (d, J=8.0 Hz, 1H), 7.15 (s, 1H), 4.48-4.38 (m, 1H), 3.73 (t, J=6.0 Hz, 2H), 3.18-3.15 (m, 1H), 2.83-2.80 (m, 1H), 2.74-2.61 (m, 2H), 2.49-2.44 (m, 6H), 2.00-1.81 (m, 4H), 1.80-1.58 (m, 4H), three exchangeable protons not observed.

### Example 21

### Preparation of Compound I-206

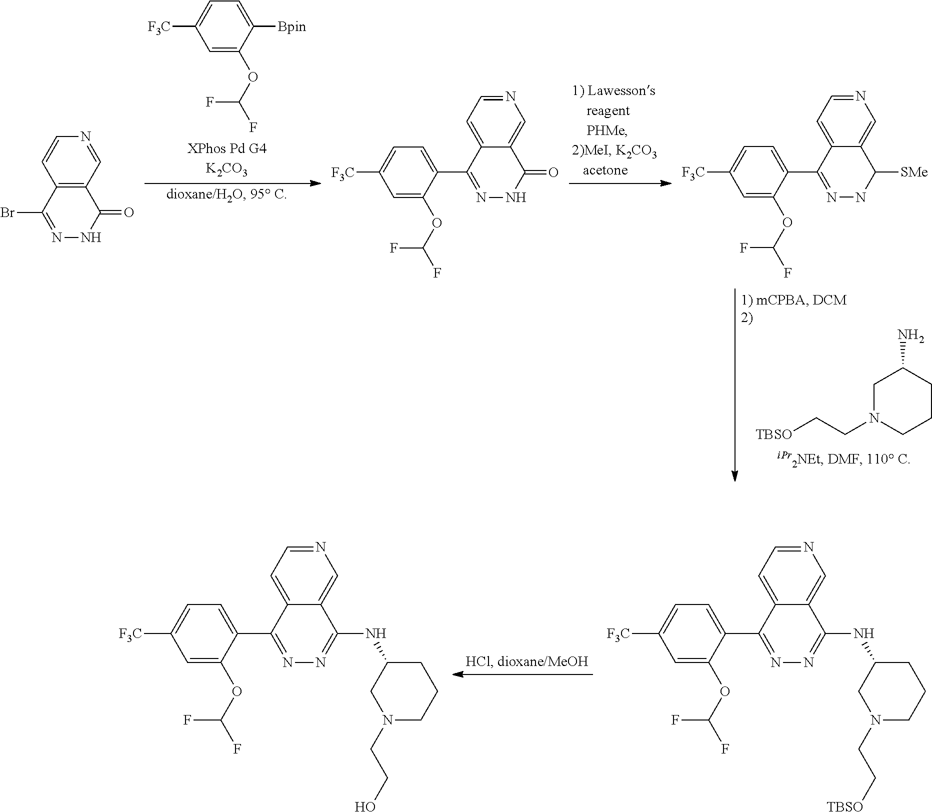

### Step 1: 1-[2-(Difluoromethoxy)-4-(trifluoromethyl)phenyl]-3H-pyrido[3,4-d]pyridazin-4-one

1-Bromo-N-pyridazin-4-one (Intermediate 1b, 0.500 g, 2.21 mmol), 2-[2-(difluoromethoxy)-4-methyl-phenyl]-4,4,5,5-tetramethyl-1,3,2-dioxaborolane (0.860 g, 2.54 mmol), and XPhos Pd G4 (0.200 g, 0.221 mmol) were added to a vial and evacuated and refilled with Ar. The mixture was dissolved in dioxane (11.0 mL) and aqueous K₂CO₃ (2 M, 3.3 mL, 6.64 mmol). The mixture was sparged with Ar for 5 min then then heated at 95° C. for 2.5 h, diluted with EtOAc and filtered through Celite. The filtrate was washed with brine and concentrated. Purification by chromatography on SiO₂ (EtOAc:hexanes, 10 to 75%) gave a white solid (0.569 g, 72%). MS m/z 358.0 [M+H]⁺; ¹H NMR (400 MHz, DMSO-d₆) δ 13.37 (s, 1H), 9.53 (s, 1H), 8.97 (d, J=5.50 Hz, 1H), 7.84 (s, 2H), 7.78 (d, 1H), 7.37 (t, J=72.54 Hz, 1H), 7.28 (d, J=5.38 Hz, 1H).

### Step 2. 1-[2-(Difluoromethoxy)-4-(trifluoromethyl)phenyl]-3H-pyrido[3,4-d]pyridazine-4-thione

To 1-[2-(difluoromethoxy)-4-(trifluoromethyl)phenyl]-3H-pyrido[3,4-d]pyridazin-4-one (0.393 g, 1.10 mmol) in PhMe (4.4 mL) was added Lawesson's reagent (0.298 g, 0.715 mmol). The mixture was heated at 95° C. for 3 h, then cooled to rt and filtered to give a pale-yellow solid (0.401 g, 98%). MS m/z 374.0 [M+H]⁺.

### Step 3: 1-[2-(Difluoromethoxy)-4-(trifluoromethyl)phenyl]-4-methylsulfanyl-pyrido[3,4-d]pyridazine

To a solution of 1-[2-(difluoromethoxy)-4-(trifluoromethyl)phenyl]-3H-pyrido[3,4-d]pyridazine-4-thione (0.190 g, 0.509 mmol) in acetone (2 mL) was added MeI (0.033 mL, 0.534 mmol) and K₂CO₃ (0.091 g, 0.662 mmol). The mixture was stirred at rt for 3.5 h, then diluted with EtOAc, washed with brine, dried (Na₂SO₄), filtered, and concentrated. Purification by chromatography on SiO₂ (EtOAc:hexanes, 20-60%) gave a white solid (0.137 g, 70%). MS m/z 388.0 [M+H]⁺; ¹H NMR (400 MHz, CDCl₃) δ 9.71 (d, J=1.00 Hz, 1H), 9.00 (d, J=5.75 Hz, 1H), 7.75 (s, 2H), 7.67 (s, 1H), 7.43 (dd, J=5.75 Hz, 1.00 Hz, 1H), 6.45 (t, J=72.42 Hz, 1H), 2.95 (s, 3H).

### Step 4: N-[(3R)-1-[2-[tert-Butyl(dimethyl)silyl]oxyethyl]-3-piperidyl]-1-[2-(difluoromethoxy)-4-(trifluoromethyl)phenyl]pyrido[3,4-d]pyridazin-4-amine

A solution of 1-[2-(difluoromethoxy)-4-(trifluoromethyl)phenyl]-4-methylsulfanyl-pyrido[3,4-d]pyridazine (0.0750 g, 0.19747 mmol) in DCM (2 mL) was cooled to 0° C. m-CPBA (0.058 g, 0.252 mmol) in DCM (1.5 mL) was added dropwise. After 30 min the reaction was warmed to rt and stirred for 2 h. The solution was diluted with DCM and washed with sat. NaHCO₃, water, brine, dried (MgSO4), filtered and concentrated to give a mixture of sulfoxide and sulfone. The crude material was dissolved in DMF (1.0 mL), to which was added (3R)-1-[2-[t-butyl(dimethyl)silyl]oxyethyl]piperidin-3-amine (0.062 g, 0.24 mmol) and iPr₂NEt (0.084 mL, 0.48 mmol). The mixture was stirred at 110° C. for 16 h, then cooled to rt and diluted with EtOAc. The organic phase was washed with water and brine, dried (Na₂SO₄), filtered and concentrated. Purification by chromatography on SiO₂ (1M NH₄OH in MeOH:DCM, 0 to 20%) gave a yellow solid. MS m/z 598.7 [M+H]⁺.

### Step 5: 2-[(3R)-3-[[1-[2-(Difluoromethoxy)-4-(trifluoromethyl)phenyl]pyrido[3,4-d]pyridazin-4-yl]amino]-1-piperidyl]ethanol formic acid salt

To a solution of N-[(3R)-1-[2-[tert-butyl(dimethyl)silyl]oxyethyl]-3-piperidyl]-1-[2-(difluoromethoxy)-4-(trifluoromethyl)phenyl]pyrido[3,4-d]pyridazin-4-amine in MeOH (0.5 mL) was added 4M HCl/dioxane (1.0 mL). The mixture was stirred at rt for 3 h, then diluted with DCM/iPrOH (9:1) and washed with saturated NaHCO₃, water, and brine. The organics were dried (Na₂SO₄), filtered, and concentrated to give a brown oil. Purification by reverse phase chromatography (0.1% formic acid in MeCN:0.1% formic acid in H₂O, 5 to 100%) gave a white solid (0.011 g, 10%). MS m/z 484.5[M+H]⁺; ¹H NMR (400 MHz, DMSO-d₆) δ 9.82 (s, 1H), 8.86 (d, J=5.6 Hz, 1H), 8.18 (s, 1H, formic acid), 7.87-7.70 (m, 4H), 7.35 (t, J=73.29 Hz, 1H), 7.27 (d, J=5.88 Hz, 1H), 4.55-4.41 (m, 2H), 3.53 (t, J=5.6 Hz, 2H), 3.22-3.12 (m, 1H), 2.89-2.77 (m, 1H), 2.48-2.43 (m, 2H), 2.20-1.95 (m, 3H), 1.81-1.72 (m, 1H), 1.68-1.44 (m, 2H).

### Example 22

### Preparation of Compounds I-292 and I-297

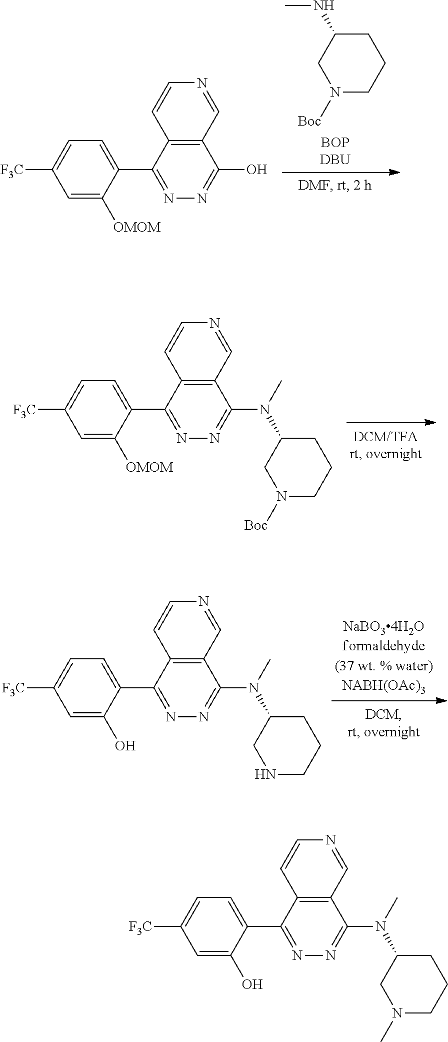

### Step 1. tert-Butyl (R)-3-((1-(2-(methoxymethoxy)-4-(trifluoromethyl)phenyl)pyrido[3,4-d]pyridazin-4-yl)(methyl)amino)piperidine-1-carboxylate

To a solution of 1-[2-(methoxymethoxy)-4-(trifluoromethyl)phenyl]pyrido[3,4-d]pyridazin-4-ol (Intermediate 14a, 400 mg, 1.14 mmol, 1.0 eq.), BOP (623 mg, 1.37 mmol, 1.2 eq.), 1,8-diazabicyclo[5.4.0]undec-7-ene (0.43 mL, 2.85 mmol, 2.5 eq.) in DMF (0.2 M) was added tert-butyl (3R)-3-(methylamino)piperidine-1-carboxylate (1.46 g, 6.83 mmol, 3.0 eq.) The mixture was allowed to stir at room temperature for 2 hours. Upon completion, the mixture was diluted with EtOAc, washed with water and brine. The organic phase was dried over sodium sulphate, filtered, and concentrated under reduced pressure. The crude residue was purified by silica gel column chromatography eluting with 0-50% EtOAc in DCM to afford tert-butyl (R)-3-((1-(2-(methoxymethoxy)-4-(trifluoromethyl)phenyl)pyrido[3,4-d]pyridazin-4-yl)(methyl)amino)piperidine-1-carboxylate (623 mg). The impure isolated material was used in next step without further purification. MS m/z 548.3 [M+H]⁺.

### Step 2. (R)-2-(4-(Methyl(piperidin-3-yl)amino)pyrido[3,4-d]pyridazin-1-yl)-5-(trifluoromethyl)phenol

The crude from step 1 was dissolved in 5 ml DCM and 5 ml TFA at rt. The reaction was allowed to stir overnight. Upon completion, the solvent was removed under reduced pressure. The crude residue was purified by reverse phase column (MeCN/water with 0.1% formic acid) to afford (R)-2-(4-(methyl(piperidin-3-yl)amino)pyrido[3,4-d]pyridazin-1-yl)-5-(trifluoromethyl)phenol (131.9 mg, 29% over two steps) as a pale yellow solid. MS m/z 404.3 [M+H]⁺. ¹H NMR (400 MHz, CD₃OD) δ 9.68 (s, 1H), 8.94 (s, 1H), 7.73-7.52 (m, 2H), 7.45-7.22 (m, 2H), 4.71-4.61 (m, 1H), 3.94-3.72 (m, 1H), 3.51-3.35 (m, 5H), 3.09-2.98 (m, 1H), 2.32-2.11 (m, 3H), 2.01-1.81 (m, 1H), 1 NH and 1 OH not observed.

### Step 3. (R)-2-(4-(Methyl(1-methylpiperidin-3-yl)amino)pyrido[3,4-d]pyridazin-1-yl)-5-(trifluoromethyl)phenol

To a mixture of (R)-2-(4-(methyl(piperidin-3-yl)amino)pyrido[3,4-d]pyridazin-1-yl)-5-(trifluoromethyl)phenol (45 mg, 0.11 mmol, 1.0 eq.) and sodium perborate tetrahydrate (51.4 mg, 0.33 mmol, 3 eq.) in DCM (0.1 M) was added formaldehyde (37 wt. % in water, 0.03 mL, 0.33 mmol, 3 eq.) followed by sodium triacetoxyborohydride (71 mg, 0.33 mmol, 3 eq.). The mixture was stirred at room temperature overnight. Upon completion, the mixture was diluted with EtOAc and washed with water and brine. The organic phase was dried over sodium sulphate, filtered, and concentrated under reduced pressure. The crude residue was purified by reverse phase column (MeCN/water with 0.1% formic acid) to give (R)-2-(4-(methyl(1-methylpiperidin-3-yl)amino)pyrido[3,4-d]pyridazin-1-yl)-5-(trifluoromethyl)phenol (3.6 mg, 7.7%) as a yellow solid. MS m/z 418.2 [M+H]⁺; ¹H NMR (400 MHz, CD₃OD) δ 9.61 (s, 1H), 8.91 (s, 1H), 8.01-7.50 (m, 2H), 7.44-7.09 (m, 2H), 4.62-4.21 (m, 1H), 3.55-3.25 (m, 4H), 3.15-2.97 (m, 1H), 2.85-2.62 (m, 1H), 2.50 (s, 3H), 2.38-2.21 (m, 1H), 2.21-2.09 (m, 1H), 2.04-1.87 (m, 2H), 1.83-1.68 (m, 1H), 1 OH not observed.

### Example 23

### Preparation of Compounds I-219 and I-215

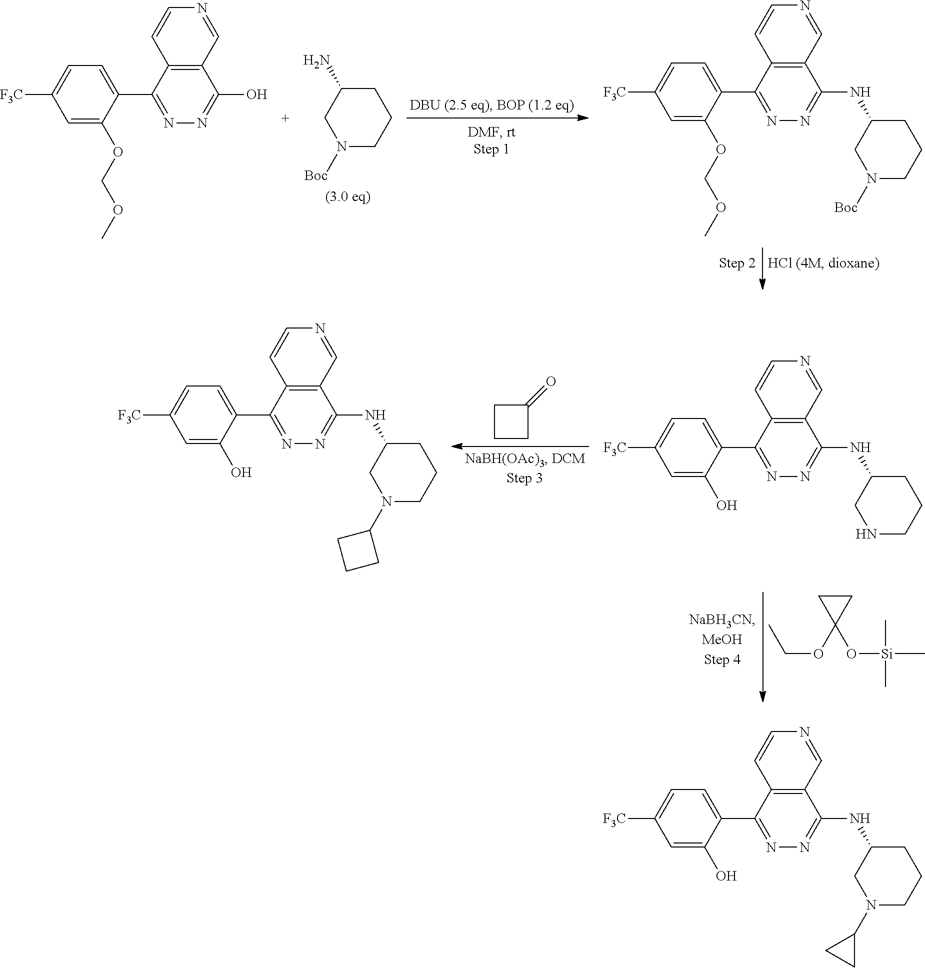

### Step 1. tert-Butyl (3R)-3-[[1-[2-(methoxymethoxy)-4-(trifluoromethyl)phenyl]pyrido[3,4-d]pyridazin-4-yl]amino]piperidine-1-carboxylate

To a mixture of 1-[2-(methoxymethoxy)-4-(trifluoromethyl)phenyl]pyrido[3,4-d]pyridazin-4-ol (Intermediate 14b, 50 mg, 0.14 mmol) and tert-butyl (3R)-3-aminopiperidine-1-carboxylate (86 mg, 3.0 eq.) in DBU (57 mg, 2.5 eq.) and DMF (0.7 mL) was added BOP (72 mg, 1.1 eq.). The reaction was stirred at rt overnight. The reaction was quenched with EtOAc and water. The organic phase was washed with H₂O followed by brine. The organic phase was dried over MgSO₄, filtered and concentrated in vacuo. The crude material was purified by flash column chromatography on silica gel eluting with 0-30% EtOAc in hexane provide the desired product which was further purified on prep-HPLC with 10-100% ACN in water with 0.1% formic acid to provide tert-butyl (3R)-3-[[1-[2-(methoxymethoxy)-4-(trifluoromethyl)phenyl]-pyrido[3,4-d]pyridazin-4-yl]amino]piperidine-1-carboxylate (43 mg, 57% yield). MS m/z 534.2 [M+H]⁺.

### Step 2. 2-[4-[[(3R)-3-Piperidyl]amino]pyrido[3,4-d]pyridazin-1-yl]-5-(trifluoromethyl)phenol hydrochloride

tert-Butyl (3R)-3-[[1-[2-(methoxymethoxy)-4-(trifluoromethyl)phenyl]pyrido[3,4-d]pyridazin-4-yl]amino]piperidine-1-carboxylate (100 mg, 0.19 mmol) was stirred in a solution of HCl (4 M in dioxane, 1 mL) at room temperature for 1 h. The organic volatiles were removed. The residue was triturated with diethyl ether and filtered to afford 2-[4-[[(3R)-3-piperidyl]amino]pyrido[3,4-d]pyridazin-1-yl]-5-(trifluoromethyl)phenol (80 mg, 92% yield) as hydrochloride salt. MS m/z 390.5 [M+H]⁺.

### Step 3. 2-[4-[[(3R)-1-Cyclobutyl-3-piperidyl]amino]pyrido[3,4-d]pyridazin-1-yl]-5-(trifluoromethyl)phenol (I-219)

A mixture of 2-[4-[[(3R)-3-piperidyl]amino]pyrido[3,4-d]pyridazin-1-yl]-5-(trifluoromethyl)phenol hydrochloride (35 mg, 0.076 mmol) and sodium perborate tetrahydrate (31 mg, 5.0 eq.) in DIPEA (20 mg, 2.0 eq.) and DCM (0.75 mL) was stirred for 15 mins, followed by addition of cyclobutanone (7.04 mg, 1.3 eq.) and NaBH(OAc)₃ (49 mg, 3.0 eq.). After 15 mins, the reaction was quenched by dilution with DCM and aq. NaHCO₃ (sat.). The organic phase was washed with H₂O followed by brine. The organic phase was dried over Na₂SO₄, filtered and concentrated in vacuo. The crude material was purified by flash column chromatography on silica gel eluting with 0-20% MeOH (with NH₄OH as modifier) in DCM to provide 2-[4-[[(3R)-1-cyclobutyl-3-piperidyl]amino]pyrido[3,4-d]pyridazin-1-yl]-5-(trifluoromethyl)phenol (16 mg, 48% yield). MS m/z 444.5 [M+H]⁺. ¹H NMR (CD₃OD) δ: 9.72 (s, 1H), 8.87 (d, J=5.8 Hz, 1H), 7.59 (d, J=7.9 Hz, 1H), 7.49 (d, J=5.8 Hz, 1H), 7.26-7.36 (m, 2H), 4.61-4.72 (m, 1H), 2.81-3.17 (m, 3H), 2.24-2.43 (m, 2H), 2.14-2.24 (m, 3H), 1.93-2.12 (m, 3H), 1.71-1.89 (m, 4H). NH and OH not observed.

### Step 4. (R)-2-(4-((1-Cyclobutylpiperidin-3-yl)amino)pyrido[3,4-d]pyridazin-1-yl)-5-(trifluoromethyl)phenol (I-215)

To a mixture of 2-[4-[[(3R)-3-piperidyl]amino]pyrido[3,4-d]pyridazin-1-yl]-5-(trifluoromethyl)phenol hydrochloride (product in step 2, 35 mg, 0.076 mmol) and DIPEA (10 mg, 1.0 eq.) in MeOH (1.5 mL) was added (1-ethoxycyclopropoxy)trimethylsilane (54 mg, 0.0 eq.) and sodium cyanoborohydride (10 mg, 2.0 eq.). The reaction was stirred at rt for 2 days. The reaction was quenched by dilution with DCM and aq. NaHCO₃ (sat.). The organic phase was washed with H₂O followed by brine. The organic phase was dried over Na₂SO₄, filtered and concentrated in vacuo. The crude material was purified by flash column chromatography on silica gel eluting with 0-20% MeOH (with NH₄OH as modifier) in DCM provide 2-[4-[[(3R)-1-cyclopropyl-3-piperidyl]amino]pyrido[3,4-d]pyridazin-1-yl]-5-(trifluoromethyl)phenol (15 mg, 46% yield). MS m/z 430.5 [M+H]⁺. ¹H NMR (CD₃OD) δ: 9.56-9.60 (m, 1H), 8.74 (d, J=5.8 Hz, 1H), 7.47 (d, J=7.9 Hz, 1H), 7.37 (dd, J=5.7, 0.8 Hz, 1H), 7.13-7.24 (m, 2H), 4.42-4.54 (m, 1H), 3.23-3.30 (m, 1H), 2.79-2.89 (m, 1H), 2.30-2.43 (m, 2H), 1.97-2.08 (m, 1H), 1.69-1.78 (m, 1H), 1.52-1.69 (m, 3H), 0.36-0.48 (m, 4H). NH and OH not observed.

### Example 24

### Preparation of Compounds I-266 and I-318

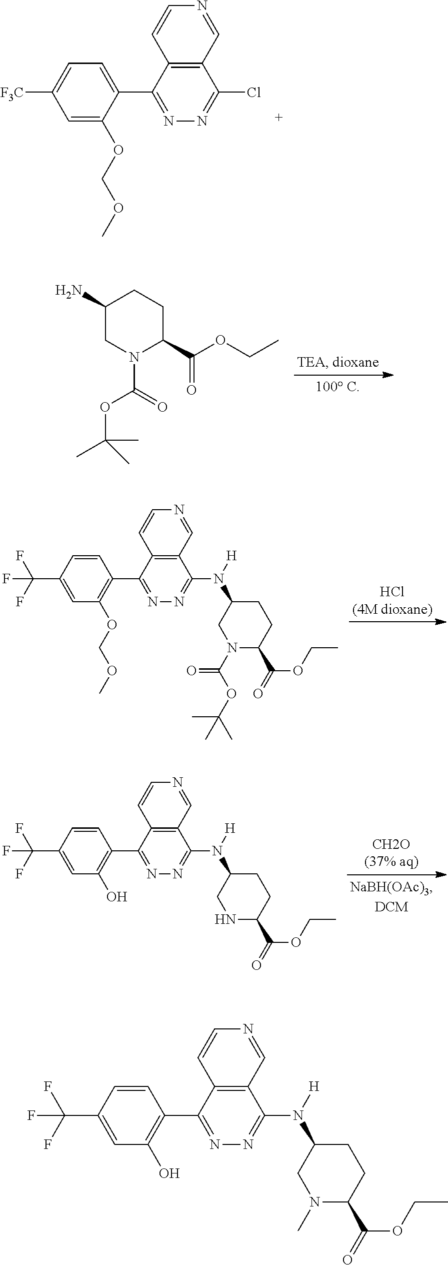 [SMILES: COCOc1cc(C(F)(F)F)ccc1-c1nnc(Cl)c2cnccc12 ; CCOC(=O)[C@@H]1CC[C@H](N)CN1C(=O)OC(C)(C)C ; CCOC(=O)[C@@H]1CC[C@H](Nc2nnc(-c3ccc(C(F)(F)F)cc3OCOC)c3ccncc23)CN1C(=O)OC(C)(C)C ; CCOC(=O)[C@@H]1CC[C@H](Nc2nnc(-c3ccc(C(F)(F)F)cc3O)c3ccncc23)CN1 ; CCOC(=O)[C@@H]1CC[C@H](Nc2nnc(-c3ccc(C(F)(F)F)cc3O)c3ccncc23)CN1C]

### Step 1. 1-(tert-Butyl) 2-ethyl (2S,5R)-5-((1-(2-(methoxymethoxy)-4-(trifluoromethyl)phenyl)pyrido[3,4-d]pyridazin-4-yl)amino)piperidine-1,2-dicarboxylate

A mixture of 4-chloro-1-[2-(methoxymethoxy)-4-(trifluoromethyl)phenyl]pyrido[3,4-d]pyridazine (Intermediate 14b, 63 mg, 0.17 mmol) and 1-(tert-butyl) 2-ethyl (2S,5R)-5-aminopiperidine-1,2-dicarboxylate (62 mg, 1.30 eq.) in triethylamine (19 mg, 1.10 eq.) and dioxane (0.7 mL) was heated at 100° C. for 24 hr. After cooling, the reaction mixture was concentrated. The crude material was purified by flash column chromatography on silica gel eluting with 0-100% EtOAc in DCM to provide 1-(tert-butyl) 2-ethyl (2S,5R)-5-((1-(2-(methoxymethoxy)-4-(trifluoromethyl)phenyl)pyrido[3,4-d]pyridazin-4-yl)amino)piperidine-1,2-dicarboxylate (85 mg, 83% yield). MS m/z 606.6 [M+H]⁺.

### Step 2. Ethyl (2S,5R)-5-((1-(2-hydroxy-4-(trifluoromethyl)phenyl)pyrido[3,4-d]pyridazin-4-yl)amino)piperidine-2-carboxylate hydrochloride

1-(tert-Butyl) 2-ethyl (2S,5R)-5-((1-(2-(methoxymethoxy)-4-(trifluoromethyl)phenyl)pyrido[3,4-d]pyridazin-4-yl)amino)piperidine-1,2-dicarboxylate (85 mg, 0.14 mmol) was stirred in a solution of HCl (4 M in dioxane, 1 mL) at room temperature for 1 h. The organic volatiles were removed. The residue was triturated with diethyl ether and filtered to affordethyl (2S,5R)-5-[[1-[2-hydroxy-4-(trifluoromethyl)phenyl]pyrido[3,4-d]pyridazin-4-yl]amino]piperidine-2-carboxylate (65 mg, 93% yield). as hydrochloride salt. MS m/z 462.5 [M+H]⁺. ¹H NMR (CD₃OD) δ: 9.98-10.05 (m, 1H), 9.15-9.20 (m, 1H), 7.77-7.82 (m, 1H), 7.70 (br d, J=8.1 Hz, 1H), 7.45 (d, J=8.4 Hz, 1H), 7.38 (s, 1H), 4.58-4.70 (m, 1H), 4.33-4.42 (m, 2H), 4.18-4.30 (m, 1H), 3.89-3.97 (m, 1H), 3.20-3.29 (m, 1H), 2.55-2.65 (m, 1H), 2.43-2.53 (m, 1H), 1.97-2.15 (m, 2H), 1.35-1.42 (m, 3H). NH and OH not observed.

### Step 3. Ethyl (2S,5R)-5-((1-(2-hydroxy-4-(trifluoromethyl)phenyl)pyrido[3,4-d]pyridazin-4-yl)amino)-1-methylpiperidine-2-carboxylate

To a mixture of ethyl (2S,5R)-5-[[1-[2-hydroxy-4-(trifluoromethyl)phenyl]pyrido[3,4-d]pyridazin-4-yl]amino]piperidine-2-carboxylate hydrochloride (20 mg, 0.040 mmol) and sodium perborate tetrahydrate (16 mg, 5.0 eq.) in DIPEA (5.3 mg, 1.0 eq.) and DCM (0.40 mL) was stirred for 15 mins, followed by addition of CH₂O (aq. 37 mass %, 6.5 mg, 2.0 eq.) and NaBH(OAc)₃ (26 mg, 3.0 eq.). After 15 mins, the reaction was quenched by dilution with DCM and NaHCO₃ (sat.). The organic phase was washed with H₂O followed by brine. The organic phase was dried over Na₂SO₄, filtered and concentrated in vacuo. The crude material was purified by flash column chromatography on silica gel eluting with 0-20% MeOH (with NH₄OH as modifier) in DCM to provide ethyl (2S,5R)-5-[[1-[2-hydroxy-4-(trifluoromethyl)phenyl]-pyrido[3,4-d]pyridazin-4-yl]amino]-1-methyl-piperidine-2-carboxylate (10 mg, 52% yield). MS m/z 476.5 [M+H]⁺. ¹H NMR (CD₃OD) δ: 9.69 (s, 1H), 8.86 (d, J=5.6 Hz, 1H), 7.59 (d, J=7.9 Hz, 1H), 7.49 (d, J=5.5 Hz, 1H), 7.33 (br d, J=7.9 Hz, 1H), 7.28 (s, 1H), 4.60-4.72 (m, 1H), 4.25 (q, J=7.2 Hz, 2H), 3.40-3.51 (m, 1H), 2.85 (br d, J=9.8 Hz, 1H), 2.30-2.38 (m, 4H), 2.22-2.28 (m, 1H), 2.04-2.16 (m, 1H), 1.78-1.93 (m, 1H), 1.54-1.69 (m, 1H), 1.29-1.35 (t, J=7.2 Hz, 3H). NH and OH not observed.

The compounds below were prepared according to the procedure of Example 24 by substituting the appropriate starting materials, reagents and reaction conditions.

| Compound | Spectral Data |
|---|---|
| I-19 | MS m/z 421.2 [M + H]⁺ |
| | ¹H NMR (400 MHz, CD₃OD) δ 7.37-7.32 (m, 1H), 7.20 (dd, J = 8.0, 1.2 Hz, |
| | 1H), 7.17-7.14 (m, 1H), 4.48-4.37 (m, 1H), 3.23-3.09 (m, 1H), 2.88-2.71 |
| | (m, 1H), 2.64-2.51 (m, 2H), 2.49-2.42 (m, 4H), 2.41-2.24 (m, 2H), 2.06- |
| | 1.95 (m, 1H), 1.93-1.80 (m, 3H), 1.78-1.67 (m, 3H), 1.65-1.54 (m, 1H), |
| | 1.15 (t, J = 7.2 Hz, 3H), NH and OH not observed. |
| I-26 | MS m/z 393.1 [M + H]⁺; ¹H NMR (400 MHz, CDCl₃) δ 7.50 (d, J = 8.0 Hz, 1H), |
| | 7.34 (d, J = 1.6 Hz, 1H), 7.13 (dd, J = 8.0, 1.6 Hz, 1H), 5.87 (s, 1H), 5.15-5.05 |
| | (m, 1H), 3.63-3.53 (m, 1H), 3.45-3.35 (m, 1H), 3.00-2.91 (m, 1H), 2.79- |
| | 2.60 (m, 7H), 2.65-2.54 (m, 2H), 2.20-2.10 (m, 2H), 1.98-1.89 (m, 2H), |
| | 1.73-1.65 (m, 2H). |
| I-34 | MS m/z 435.0 [M + H]⁺; ¹H NMR (400 MHz, MeOD-d4) δ 7.34 (d, J = 7.8 Hz, |
| | 1H), 7.21-7.17 (dd, J = 7.8, 1.2 Hz, 1H), 7.15 (d, J = 1.2 Hz, 1H), 4.46-4.36 |
| | (m, 1H), 3.20-3.10 (m, 1H), 2.91-2.82 (m, 1H), 2.80-2.72 (m, 1H), 2.52- |
| | 2.32 (m, 6H), 2.00-1.78 (m, 4H), 1.71 m, 3H), 1.63-1.52 (m, 1H), 1.11 (d, J = |
| | 6.6 Hz, 6H), NH and OH not observed. |
| I-115 | MS m/z 417.3 [M + H]⁺; ¹H NMR (CHLOROFORM-d) δ: 8.24 (d, J = 8.0 Hz, |
| | 1H), 7.82-7.95 (m, 3H), 7.72 (d, J = 8.0 Hz, 1H), 7.42 (s, 1H), 7.23 (d, J = 8.4 Hz, |
| | 1H), 6.67 (br d, J = 3.8 Hz, 1H), 3.77 (br t, J = 5.8 Hz, 2H), 2.78 (t, J = 5.8 Hz, 2H), |
| | 2.54 (br s, 4H), 1.67 (quin, J = 5.5 Hz, 4H), 1.25 (s, 2H), 0.88 (br t, J = 6.5 Hz, |
| | 1H). |
| I-116 | MS m/z 390.1 [M + H]⁺; ¹H NMR (DMSO-d6) δ: 10.66 (br s, 1H), 8.94-8.99 (m, |
| | 2H), 7.96 (dd, J = 5.8, 0.8 Hz, 1H), 7.66 (d, J = 7.8 Hz, 1H), 7.35-7.38 (m, 1H), |
| | 7.33 (s, 1H), 3.49-3.61 (m, 4H), 2.58-2.71 (m, 4H), 2.31 (s, 3H). |
| I-147 | MS m/z 433.0 [M + H]⁺; ¹H NMR (DMSO-d6) δ: 10.26-10.52 (br s, 1H), 8.28 (d, |
| | J = 8.3 Hz, 1H), 7.87 (t, J = 7.2 Hz, 1H), 7.78 (t, J = 7.3 Hz, 1H), 7.49-7.57 (m, |
| | 2H), 7.45 (d, J = 7.8 Hz, 1H), 7.25-7.33 (m, 2H), 3.75-3.84 (m, 1H), 3.66-3.75 |
| | (m, 2H), 3.61 (br dd, J = 11.0, 3.0 Hz, 1H), 3.49-3.55 (m, 1H), 3.11 (br dd, |
| | J = 10.9, 8.9 Hz, 1H), 2.98-3.06 (m, 1H), 2.85 (br d, J = 11.8 Hz, 1H), 2.55-2.68 |
| | (m, 1H), 2.44-2.48 (m, 1H), 0.98 (d, J = 6.3 Hz, 3H). |
| I-148 | MS m/z 433.0 [M + H]⁺; ¹H NMR (METHANOL-d4) δ: 8.20 (d, J = 8.3 Hz, 1H), |
| | 7.89 (t, J = 7.3 Hz, 1H), 7.81 (t, J = 7.6 Hz, 1H), 7.61 (d, J = 7.8 Hz, 1H), 7.53 (d, |
| | J = 7.8 Hz, 1H), 7.30 (d, J = 7.8 Hz, 1H), 7.25 (s, 1H), 3.65-3.93 (m, 5H), 3.20- |
| | 3.29 (m, 2H), 3.04 (dt, J = 12.0, 2.8 Hz, 1H), 2.74 (ddd, J = 12.8, 7.0, 5.3 Hz, 1H), |
| | 2.63-2.70 (m, 1H), 2.59 (ddd, J = 12.0, 10.0, 3.3 Hz, 1H), 1.08 (d, J = 6.5 Hz, 3H). |
| | NH and OH not observed |
| I-149 | MS m/z 418.2 [M + H]⁺; ¹H NMR (METHANOL-d4) δ: 8.23 (d, J = 8.0 Hz, 1H), |
| | 7.89 (ddd, J = 8.3, 7.2, 1.1 Hz, 1H), 7.81 (td, J = 7.7, 1.3 Hz, 1H), 7.61 (d, J = 7.8 |
| | Hz, 1H), 7.52 (d, J = 8.0 Hz, 1H), 7.30 (d, J = 7.6 Hz, 1H), 7.25 (s, 1H), 3.86 (t, |
| | J = 6.4 Hz, 2H), 3.16-3.24 (m, 4H), 2.83-2.92 (m, 6H). NH and OH not observed |
| I-150 | MS m/z 375.0 [M + H]⁺; ¹H NMR (400 MHz, CD₃OD) δ 8.28-8.22 (m, 1H), |
| | 7.97-7.80 (m, 2H), 7.67-7.62 (m, 1H), 7.50 (d, J = 8.0 Hz, 1H), 7.31-7.22 |
| | (m, 2H), 4.82-4.71 (m, 1H), 4.17-4.06 (m, 1H), 4.04-3.86 (m, 3H), 2.67- |
| | 2.46 (m, 2H), OH and NHs not observed. |
| I-152 | MS m/z 418.0 [M + H]⁺; ¹H NMR (400 MHz, DMSO-d6) δ 10.8 (bs, 1H), 8.98 |
| | (s, 2H), 7.95 (d, J = 4.0 Hz, 1H), 7.65 (d, J = 4.0 Hz, 1H), 7.38-7.36 (m, 2H), |
| | 4.16-4.15 (m, 1H), 3.90-3.87 (m, 1H), 3.08-3.03 (m, 3H), 2.50-2.47 (m, 6H, |
| | overlapped with d-DMSO peak), 2.12-2.10 (m, 1H), 1.99-1.97 (m, 1H), 1.93- |
| | 1.91 (m, 1H), 1.83-1.80 (m, 1H). |
| I-153 | MS m/z 390.0 [M + H]⁺; ¹H NMR (DMSO-d6) δ: 8.97 (d, J = 5.8 Hz, 1H), 8.89 (s, |
| | 1H), 7.97 (d, J = 5.5 Hz, 1H), 7.70 (d, J = 7.8 Hz, 1H), 7.50-7.58 (m, 2H), 3.80 (s, |
| | 3H), 3.44-3.47 (m, 1H), 3.34 (br s, 4H), 3.00 (br s, 4H). |
| I-161 | MS m/z 432.0 [M + H]⁺; ¹H NMR (CHLOROFORM-d) δ: 8.25 (d, J = 8.0 Hz, |
| | 1H), 7.79-8.01 (m, 3H), 7.72 (d, J = 8.0 Hz, 1H), 7.43 (s, 1H), 7.24 (br d, J = 8.3 |
| | Hz, 1H), 6.34-6.58 (m, 1H), 3.81 (br t, J = 5.3 Hz, 2H), 2.84 (br t, J = 5.8 Hz, 2H), |
| | 2.44-2.71 (m, 6H), 2.29-2.40 (m, 3H). |
| I-162 | MS m/z 432.0 [M + H]⁺; ¹H NMR (400 MHz, CHLOROFORM-d) δ 8.98 (s, |
| | 1H), 8.90 (d, J = 4.0 Hz, 1H), 7.78 (d, J = 4.0 Hz, 1H), 7.68 (d, J = 8.0 Hz, 1H), |
| | 7.44 (d, J = 4.0 Hz, 1H), 7.27 (d, J = 8.0 Hz, 1H), 4.25 (d, J = 12.0 Hz, 1H), 4.00 |
| | (d, J = 12.0 Hz, 1H), 3.78 (s, 3H), 3.06-3.03 (m, 2H), 2.75-2.70 (m, 1H), 2.40 (s, |
| | 6H), 2.19-2.16 (m, 1H), 1.99-1.97 (m, 1H), 1.89-1.85 (m, 1H), 1.59-1.55 (m, |
| | 1H). |
| I-174 | MS m/z 401.1 [M + H]⁺; ¹H NMR (400 MHz, DMSO-d6) δ 10.44 (s, 1H), 9.73 |
| | (s, 1H), 8.84 (d, J = 5.6 Hz, 1H), 8.39 (t, J = 5.4 Hz, 1H), 7.72 (s, 1H), 7.57 (d, |
| | J = 7.8 Hz, 1H), 7.48 (s, 1H), 7.35-7.25 (m, 3H), 4.68 (d, J = 5.1 Hz, 2H), |
| | 3.80 (s, 3H) |
| I-180 | MS m/z 387.1 [M + H]⁺; ¹H NMR (400 MHz, DMSO-d6) δ 10.48 (s, 1H), 10.00 |
| | (s, 1H), 9.92 (s, 1H), 8.90 (d, J = 5.6 Hz, 1H), 8.35 (s, 1H), 7.75 (s, 1H), 7.60 |
| | (d, J = 8.0 Hz, 1H), 7.38-7.30 (m, 3H), 3.89 (s, 3H) |
| I-199 | MS m/z 430.8 [M + H]⁺; ¹H NMR (400 MHz, CD₃OD) δ 9.80 (d, J = 1.0 Hz, |
| | 1H), 8.83 (d, J = 5.7 Hz, 1H), 7.61 (d, J = 7.7 Hz, 1H), 7.49-7.42 (m, 2H), |
| | 7.33 (dd, J = 5.7, 1.0 Hz, 1H), 4.49 (s, 1H), 3.81 (d, J = 1.9 Hz, 1H), 3.80 (s, |
| | 3H), 3.50 (ddd, J = 14.0, 9.5, 2.4 Hz, 1H), 3.17-3.03 (m, 1H), 2.99-2.84 (m, |
| | 3H), 2.34 (d, J = 4.1 Hz, 1H), 2.05 (tdd, J = 14.7, 5.5, 2.9 Hz, 1H), 1.93-1.77 |
| | (m, 2H), 1.67-1.53 (m, 1H). NH and OH not observed. |
| I-203 | MS m/z 417.2 [M + H]⁺; ¹H NMR (400 MHz, METHANOL-d4) δ = 8.44 (d, J = |
| | 8.3 Hz, 1H), 7.90-7.76 (m, 2H), 7.65 (d, J = 8.0 Hz, 1H), 7.56 (d, J = 7.8 Hz, |
| | 1H), 7.31 (d, J = 8.0 Hz, 1H), 7.28-7.25 (m, 1H), 4.13-3.94 (m, 3H), 3.77 |
| | (dd, J = 8.1, 10.4 Hz, 1H), 2.68-2.46 (m, 3H), 2.34 (s, 6H), 2.32-2.24 (m, |
| | 1H), 1.83 (qd, J = 8.7, 11.9 Hz, 1H); OH wasn't observed |
| I-212 | MS m/z 430.1 [M + H]⁺; ¹H NMR (400 MHz, CD₃OD) δ 9.93 (d, J = 1.0 Hz, |
| | 1H), 9.10 (d, J = 5.6 Hz, 1H), 7.72-7.56 (m, 2H), 7.37 (dd, J = 8.3, 1.7 Hz, |
| | 1H), 7.31 (d, J = 1.7 Hz, 1H), 4.58 (tt, J = 11.3, 4.5 Hz, 1H), 3.64 (d, J = 13.1 |
| | Hz, 2H), 3.09 (s, 3H), 2.72 (t, J = 13.0 Hz, 1H), 2.46 (d, J = 14.2 Hz, 1H), 2.35 |
| | (dd, J = 17.1, 11.3 Hz, 1H), 1.79 (s, 1H), 1.39-1.25 (m, 2H), 1.22-1.10 (m, |
| | 1H), 1.01 (d, J = 9.5 Hz, 1H). NH and OH not observed. |
| I-213 | MS m/z 444.7 [M + H]⁺; ¹H NMR (400 MHz, CD₃OD) δ 9.62 (dd, J = 7.9, 1.0 |
| | Hz, 1H), 8.82 (dd, J = 11.8, 5.7 Hz, 1H), 7.61 (dd, J = 8.1, 1.7 Hz, 1H), 7.54- |
| | 7.40 (m, 2H), 7.32 (ddd, J = 11.7, 5.7, 1.0 Hz, 1H), 4.40 (t, J = 6.4 Hz, 1H), |
| | 3.80 (d, J = 1.1 Hz, 3H), 3.30 (s, 1H), 3.24 (d, J = 4.3 Hz, 1H), 2.36 (d, J = 12.1 |
| | Hz, 3H), 2.30 (td, J = 7.3, 3.2 Hz, 1H), 2.23-2.05 (m, 5H), 1.96-1.77 (m, |
| | 2H). NH and OH not observed. |
| I-224 | MS m/z 404.8 [M + H]⁺; ¹H NMR (400 MHz, METHANOL-d4) δ = 9.77 (br s, |
| | 1H), 8.89 (d, J = 5.8 Hz, 1H), 7.59 (d, J = 7.9 Hz, 1H), 7.51 (d, J = 5.8 Hz, 1H), |
| | 7.35 (d, J = 7.9 Hz, 1H), 7.32-7.28 (m, 1H), 4.70-4.52 (m, 1H), 3.75-3.58 |
| | (m, 2H), 2.96 (s, 3H), 2.52 (br s, 2H), 2.13 (br s, 2H); 2CH was overlapped |
| | with solvent peak. OH and NH wasn't observed. |
| I-230 | MS m/z 406.4 [M + H]⁺. ¹H NMR (400 MHz, CD₃OD) δ 9.00-8.93 (m, 2H), |
| | 8.50 (s, 1H), 8.19 (d, J = 5.8 Hz, 1H), 7.63 (d, J = 7.9 Hz, 1H), 7.36 (d, J = 7.9 |
| | Hz, 1H), 7.29 (s, 1H), 4.85-4.78 (m, 1H), 4.20 (dd, J = 12.8, 5.1 Hz, 1H), 4.10- |
| | 3.96 (m, 3H), 3.55 (d, J = 5.0 Hz, 2H), 3.30-3.25 (m, 2H). |
| I-233 | MS m/z 404.8 [M + H]⁺; ¹H NMR (400 MHz, METHANOL-d4) δ = 9.57 (s, |
| | 1H), 8.91 (d, J = 5.6 Hz, 1H), 7.64-7.56 (m, 2H), 7.40-7.34 (m, 1H), 7.34- |
| | 7.28 (m, 1H), 4.39 (br d, J = 12.1 Hz, 1H), 4.08 (br d, J = 13.0 Hz, 1H), 3.43- |
| | 3.37 (m, 3H), 2.74 (s, 6H), 2.30 (br d, J = 10.1 Hz, 1H), 2.14-1.91 (m, 2H), |
| | 1.91-1.72 (m, 1H); OH wasn't observed |
| I-238 | MS m/z 483.9 [M + H]⁺; ¹H NMR (400 MHz, DMSO-d6) δ 8.98 (d, J = 5.6 Hz, |
| | 1H), 8.81 (s, 1H), 8.35 (d, J = 5.8 Hz, 1H), 8.16 (s, 1H, formic acid), 7.89- |
| | 7.80 (m, 2H), 7.74 (s, 1H), 7.57 (s, 1 H), 7.38 (t, J = 73.5 Hz, 1H), 4.54-4.37 |
| | (m, 2H), 3.53 (t, J = 6.2 Hz, 2H), 3.20-3.12 (m, 1H), 2.88-2.79 (m, 1H), 2.48- |
| | 2.44 (m, 2H), 2.19-1.95 (m, 3H), 1.83-1.69 (m, 1H), 1.68-1.41 (m, 2H) |
| I-243 | MS m/z 416.3 [M − H]⁻; ¹H NMR (400 MHz, METHANOL-d4) δ = 9.67 (s, 1H), |
| | 8.85 (d, J = 5.8 Hz, 1H), 7.57 (d, J = 7.9 Hz, 1H), 7.50 (d, J = 5.6 Hz, 1H), 7.33- |
| | 7.23 (m, 2H), 4.11 (dd, J = 4.0, 13.9 Hz, 1H), 3.72 (dd, J = 6.1, 13.9 Hz, 1H), |
| | 3.08-2.95 (m, 1H), 2.67-2.58 (m, 1H), 2.56 (s, 3H), 2.35 (dt, J = 4.4, 11.2 Hz, |
| | 1H), 1.94 (br dd, J = 3.0, 13.4 Hz, 1H), 1.88-1.79 (m, 1H), 1.78-1.51 (m, |
| | 3H), 1.48-1.36 (m, 1H); NH and OH wasn't observed. |
| I-251 | MS m/z 418.5 [M + H]⁺; ¹H NMR (400 MHz, CD₃OD) δ 9.69 (s, 1H), 8.88 (d, J = |
| | 5.7 Hz, 1H), 8.54 (s, 1H), 7.62 (d, J = 7.7 Hz, 1H), 7.54-7.45 (m, 2H), 7.38 |
| | (d, J = 5.7 Hz, 1H), 4.62 (s, 1H), 3.64 (dd, J = 13.8, 3.9 Hz, 1H), 3.48-3.34 |
| | (m, 2H), 3.29-3.18 (m, 1H), 2.38-2.26 (m, 1H), 2.19-1.89 (m, 4H), 1.85- |
| | 1.68 (m, 1H). |
| I-252 | MS m/z 406.4 [M + H]⁺; ¹H NMR (400 MHz, CD₃OD) δ 9.69 (s, 1H), 8.90 (d, J = |
| | 5.7 Hz, 1H), 8.47 (s, 1H), 7.58 (d, J = 7.9 Hz, 1H), 7.52 (d, J = 5.7 Hz, 1H), |
| | 7.34 (d, J = 8.0 Hz, 1H), 7.28 (s, 1H), 4.82-4.71 (m, 1H), 4.22 (dd, J = 12.7, 5.1 |
| | Hz, 1H), 4.12-3.99 (m, 3H), 3.61 (d, J = 5.0 Hz, 2H), 3.42-3.33 (m, 1H), 3.28- |
| | 3.18 (m, 1H). |
| I-253 | MS m/z 404.5 [M + H]⁺; ¹H NMR (400 MHz, CD₃OD) δ 9.70 (s, 1H), 8.87 (d, J = |
| | 5.7 Hz, 1H), 8.54 (brs, 1H), 7.57 (d, J = 7.9 Hz, 1H), 7.49 (d, J = 5.7 Hz, 1H), |
| | 7.33 (d, J = 8.0 Hz, 1H), 7.27 (s, 1H), 4.65-4.53 (m, 1H), 3.53-3.35 (m, 3H), |
| | 3.30-3.21 (m, 1H), 2.57-2.32 (m, 2H), 2.23-2.10 (m, 2H), 2.04-1.90 (m, |
| | 2H). |
| I-257 | MS m/z 508.2 [M + H]⁺; ¹H NMR (500 MHz, CD₃OD) δ 9.82 (s, 1H), 9.01 (s, |
| | 1H), 7.62 (d, J = 7.3 Hz, 2H), 7.44-7.24 (m, 2H), 6.07-5.73 (m, 1H), 5.49 (d, |
| | J = 7.5 Hz, 1H), 4.67 (s, 1H), 3.95 (s, 1H), 3.80-3.46 (m, 2H), 2.97-2.66 (m, |
| | 2H), 2.60 (d, J = 16.9 Hz, 2H), 2.48-2.12 (m, 3H), 2.03 (s, 5H), NH and OH |
| | not observed. |
| I-260 | MS m/z 420.4 [M + H]⁺; ¹H NMR (400 MHz, CD₃OD) δ 8.99-8.89 (m, 2H), |
| | 8.19 (d, J = 5.7 Hz, 1H), 7.62 (d, J = 7.9 Hz, 1H), 7.38-7.31 (m, 1H), 7.28 (s, |
| | 1H), 4.86-4.78 (m, 1H), 4.15 (dd, J = 12.6, 4.8 Hz, 1H), 3.98 (dd, J = 12.7, 5.4 |
| | Hz, 1H), 3.93-3.78 (m, 2H), 3.12-2.98 (m, 2H), 2.91-2.76 (m, 2H), 2.52 (s, |
| | 3H). |
| I-261 | MS m/z 404.4 [M + H]⁺; ¹H NMR (400 MHz, CD₃OD) δ 9.68 (s, 1H), 8.90 (d, J = |
| | 5.7 Hz, 1H), 8.53 (s, 1H), 7.58 (d, J = 7.9 Hz, 1H), 7.51 (d, J = 5.7 Hz, 1H), |
| | 7.34 (d, J = 8.2 Hz, 1H), 7.28 (s, 1H), 4.68-4.55 (m, 1H), 3.65 (dd, J = 13.8, |
| | 4.0 Hz, 1H), 3.47-3.34 (m, 2H), 3.28-3.18 (m, 1H), 2.44-2.24 (m, 1H), |
| | 2.20-1.91 (m, 4H), 1.84-1.71 (m, 1H). |
| I-262 | MS m/z 420.4 [M + H]⁺; ¹H NMR (400 MHz, CD₃OD) δ 9.68 (s, 1H), 8.88 (s, |
| | 1H), 7.58 (d, J = 7.9 Hz, 1H), 7.53-7.46 (m, 1H), 7.33 (d, J = 7.9 Hz, 1H), |
| | 7.27 (s, 1H), 4.19 (dd, J = 12.7, 5.1 Hz, 1H), 4.97-4.87 (m, 1H), 4.01 (dd, J = |
| | 12.6, 5.9 Hz, 1H), 3.96-3.82 (m, 2H), 3.28-3.15 (m, 2H), 3.10-2.90 (m, |
| | 2H), 2.64 (s, 3H). |
| I-263 | MS m/z 418.5 [M + H]⁺; ¹H NMR (400 MHz, CD₃OD) δ 9.69 (s, 1H), 8.86 (d, J = |
| | 5.7 Hz, 1H), 8.56 (s, 1H), 7.57 (d, J = 7.9 Hz, 1H), 7.48 (d, J = 5.6 Hz, 1H), |
| | 7.32 (d, J = 8.0 Hz, 1H), 7.27 (s, 1H), 4.72-4.56 (m, 1H), 3.41-3.34 (m, 1H), |
| | 3.23 (s, 3H), 2.80 (s, 3H), 2.48-2.31 (m, 2H), 2.24-1.92 (m, 4H). |
| I-264 | MS m/z 433.5 [M + H]⁺; ¹H NMR (400 MHz, CD₃OD) δ 9.64 (s, 1H), 8.88 (d, J = |
| | 5.7 Hz, 1H), 8.52 (s, 1H, formic acid), 7.58 (d, J = 7.9 Hz, 1H), 7.50 (d, J = |
| | 5.7 Hz, 1H), 7.33 (d, J = 8.0 Hz, 1H), 7.27 (s, 1H), 4.83-4.74 (m, 1H), 3.22 |
| | (dd, J = 13.4, 4.7 Hz, 2H), 3.15 (dd, J = 13.5, 6.1 Hz, 2H), 3.06-2.87 (m, 4H), |
| | 2.58 (s, 6H). |
| I-269 | MS m/z 404.9 [M + H]⁺; ¹H NMR (400 MHz, DMSO-d6) δ 10.48 (br s, 1 H), |
| | 9.77 (s, 1 H), 8.84 (d, J = 5.63 Hz, 1 H), 8.19 (s, 1 H, formic acid), 7.76 (d, J = |
| | 7.63 Hz, 1 H), 7.55 (d, J = 7.88 Hz, 1 H), 7.34-7.25 (m, 3 H), 4.78-4.66 (m, 1 |
| | H), 4.40-4.27 (m, 1 H), 3.62-3.49 (m, 1 H), 2.29 (d, J = 11.63 Hz, 1 H), 2.11- |
| | 1.96 (m, 1 H), 1.88 (d, J = 11.01 Hz, 1 H), 1.84-1.72 (m, 1 H), 1.44-1.23 |
| | (m, 3 H), 1.21-1.06 (m, 1 H) |
| I-270 | MS m/z 404.9 [M + H]⁺; ¹H NMR (400 MHz, DMSO-d6) δ 10.49 (br s, 1 H), |
| | 9.79 (s, 1 H), 8.83 (d, J = 5.50 Hz, 1 H), 8.21 (s, 1 H, formic acid), 7.63-7.51 |
| | (m, 2 H), 7.37-7.22 (m, 3 H), 4.81-4.65 (m, 1 H), 4.56-4.44 (m, 1 H), 4.13- |
| | 4.02 (m, 1 H), 2.10-1.88 (m, 2 H), 1.86-1.67 (m, 2 H), 1.66-1.23 (m, 2 |
| | H), 1.51-1.34 (m, 2 H) |
| I-271 | MS m/z 446.8 [M + H]⁺; ¹H NMR (400 MHz, METHANOL-d4) δ = 9.72 (s, |
| | 1H), 8.86 (d, J = 5.8 Hz, 1H), 7.59 (d, J = 7.8 Hz, 1H), 7.48 (d, J = 5.6 Hz, 1H), |
| | 7.33 (d, J = 8.0 Hz, 1H), 7.30-7.26 (m, 1H), 4.74 (s, 5H), 3.59 (quin, J = 6.4 |
| | Hz, 1H), 3.19-3.05 (m, 1H), 2.68 (s, 1H), 2.23-2.03 (m, 3H), 1.97-1.86 (m, |
| | 1H), 1.85-1.62 (m, 2H); OH and NH wasn't observed |
| I-272 | MS m/z 372.1 [M − H]−; ¹H NMR (400 MHz, METHANOL-d4) δ = 9.73 (s, 1H), |
| | 8.84 (d, J = 5.6 Hz, 1H), 7.31 (d, J = 5.6 Hz, 1H), 7.20 (d, J = 7.8 Hz, 1H), 7.14 |
| | (s, 1H), 7.12-7.04 (m, 1H), 4.72-4.56 (m, 1H), 3.24-3.06 (m, 1H), 2.81- |
| | 2.65 (m, 1H), 2.38 (s, 5H), 2.07 (s, 4H), 2.04-1.95 (m, 1H), 1.95-1.85 (m, |
| | 1H), 1.85-1.60 (m, 2H), 1.09-1.00 (m, 2H), 0.82-0.74 (m, 2H); NH wasn't |
| | observed |
| I-275 | MS m/z 406.3 [M + H]⁺; ¹H NMR (400 MHz, MeOD) δ 9.71 (s, 1H), 8.90 (s, |
| | 1H), 8.55 (s, 1H, formic acid proton), 7.89-7.40 (m, 2H), 7.40-7.14 (m, 2H), |
| | 3.88 (s, 2H), 2.64 (s, 6H), 1.35 (s, 6H). 1 OH and 1 NH not observed. |
| I-277 | MS m/z 454.3 [M + H]⁺; ¹H NMR (400 MHz, CD₃OD) δ 9.94 (s, 1H), 9.11 (s, |
| | 1H), 7.65 (dd, J = 12.5, 6.6 Hz, 2H), 7.40 (d, J = 7.9 Hz, 1H), 7.33 (s, 1H), 6.69- |
| | 5.72 (m, 1H), 4.77-4.37 (m, 1H), 3.80 (d, J = 11.5 Hz, 1H), 3.75-3.40 (m, |
| | 3H), 3.07 (q, J = 12.6 Hz, 2H), 2.29 (d, J = 12.4 Hz, 1H), 2.24-2.10 (m, 1H), |
| | 2.06-1.78 (m, 2H), NH and OH not observed. |
| I-281 | MS m/z 458.2 [M − H]−; ¹H NMR (400 MHz, METHANOL-d4) δ = 9.71 (s, 1H), |
| | 8.86 (d, J = 5.6 Hz, 1H), 7.59 (d, J = 7.9 Hz, 1H), 7.48 (d, J = 5.6 Hz, 1H), 7.33 |
| | (br d, J = 8.0 Hz, 1H), 7.30-7.26 (m, 1H), 4.63 (br t, J = 8.9 Hz, 1H), 4.02- |
| | 3.84 (m, 2H), 3.83-3.69 (m, 2H), 3.25-3.09 (m, 2H), 2.89 (br d, J = 10.3 Hz, |
| | 1H), 2.45-2.26 (m, 2H), 2.21-2.07 (m, 2H), 2.03-1.85 (m, 2H), 1.85-1.60 |
| | (m, 2H); NH and OH wasn't observed |
| I-283 | MS m/z 472.3 [M + H]⁺; ¹H NMR (400 MHz, CD₃OD) δ 9.98 (s, 1H), 9.14 (d, J = |
| | 5.4 Hz, 1H), 7.65 (d, J = 5.4 Hz, 1H), 7.61 (d, J = 7.9 Hz, 1H), 7.37 (d, J = |
| | 8.0 Hz, 1H), 7.31 (s, 1H), 4.24 (tt, J = 9.0, 4.0 Hz, 1H), 3.38-3.33 (m, 1H), |
| | 3.27 (q, J = 9.7 Hz, 2H), 3.01 (dd, J = 10.1, 5.4 Hz, 1H), 2.76 (t, J = 10.2 Hz, |
| | 1H), 2.66-2.56 (m, 1H), 2.24-2.11 (m, 1H), 1.91 (dt, J = 13.0, 4.1 Hz, 1H), |
| | 1.82 (dt, J = 13.6, 10.3 Hz, 1H), 1.70 (tq, J = 11.0, 6.4 Hz, 1H), NH and OH not |
| | observed. |
| I-286 | MS m/z 462.6 [M + H]⁺; ¹H NMR (400 MHz, METHANOL-d4) δ = 9.70 (s, 1H), |
| | 8.85 (d, J = 5.6 Hz, 1H), 7.59 (d, J = 7.9 Hz, 1H), 7.49 (d, J = 5.8 Hz, 1H), 7.33 |
| | (d, J = 8.0 Hz, 1H), 7.30-7.26 (m, 1H), 4.63-4.56 (m, 1H), 3.23 (br d, J = 9.5 |
| | Hz, 1H), 2.85 (br d, J = 11.0 Hz, 1H), 2.59-2.47 (m, 2H), 2.42 (s, 2H), 2.10- |
| | 1.98 (m, 1H), 1.93-1.61 (m, 3H), 1.24 (d, J = 7.3 Hz, 6H); 3H (2OH and NH) |
| | wasn't observed |
| I-288 | MS m/z 389.4 [M + H]⁺; ¹H NMR (400 MHz, DMSO-d6) δ 10.53 (br s, 1 H), |
| | 9.79 (s, 1 H), 8.83 (d, J = 5.75 Hz, 1 H), 7.69 (d, J = 7.38 Hz, 1 H), 7.55 (d, |
| | J = 7.63 Hz, 1 H), 7.32-7.24 (m, 3 H), 4.36-4.23 (m, 1 H), 2.17-2.05 (m, 2 |
| | H), 1.88-1.75 (m, 2 H), 1.74-1.63 (m, 1 H), 1.52-1.32 (m, 4 H) 1.31-1.15 |
| | (m, 1 H) |
| I-293 | MS m/z 484.2 [M + H]⁺; ¹H NMR (400 MHz, DMSO-d6) δ 10.46 (br s, 1 H), |
| | 9.78 (s, 1 H), 8.85 (d, J = 5.00 Hz, 1 H), 8.15 (s, 1 H, formic acid), 7.65 (br d, |
| | J = 7.50 Hz, 1 H), 7.56 (d, J = 7.75 Hz, 1 H), 736-7.23 (m, 3 H), 6.68 (t, J = 76.42 |
| | Hz, 1 H), 4.52-4.41 (m, 1 H), 3.95 (t, J = 5.75 Hz, 2 H), 3.23-3.15 (m, 1 H), |
| | 2.86 (d, J = 11.01 Hz, 1 H), 2.70-2.61 (m, 2 H), 2.17-1.97 (m, 3 H), 1.83- |
| | 1.71 (m, 1 H), 1.68-1.44 (m, 2 H) |
| I-294 | MS m/z 450.3 [M + H]⁺; ¹H NMR (400 MHz, CD₃OD) δ 9.84 (s, 1H), 9.02 (s, |
| | 1H), 7.61 (d, J = 7.4 Hz, 2H), 7.37 (d, J = 8.0 Hz, 1H), 7.31 (s, 1H), 4.82-4.69 |
| | (m, 1H), 4.64 (t, J = 5.5 Hz, 1H), 4.52 (t, J = 5.5 Hz, 1H), 4.08 (s, 1H), 3.72 (s, |
| | 1H), 3.39 (td, J = 7.6, 3.4 Hz, 2H), 3.19-2.85 (m, 2H), 2.35 (s, 1H), 2.32- |
| | 2.11 (m, 3H), 2.11-1.81 (m, 2H). |
| I-295 | MS m/z 432.5 [M + H]⁺; ¹H NMR (400 MHz, DMSO-d6) δ 10.56 (br s, 1 H), |
| | 9.78 (br s, 1 H), 8.84 (d, J = 5.00 Hz, 1 H), 8.18 (s, 1 H, formic acid), 7.65 (d, |
| | J = 7.75 Hz, 1 H), 7.56 (d, J = 7.88 Hz, 1 H), 7.35-7.24 (m, 3 H), 4.51-4.39 (m, |
| | 1 H), 3.16 (d, J = 7.88 Hz, 1 H), 2.86-2.75 (m, 2 H), 2.25-2.13 (m, 2 H), 2.10- |
| | 1.99 (m, 1 H), 1.85-1.74 (m, 1 H), 1.65-1.42 (m, 2 H), 1.01 (d, J = 6.38 Hz, |
| | 6 H). |
| I-300 | MS m/z 445.3 [M + H]⁺; ¹H NMR (400 MHz, METHANOL-d4) δ = 9.72 (s, |
| | 1H), 8.85 (d, J = 5.6 Hz, 1H), 7.58 (d, J = 7.8 Hz, 1H), 7.54-7.43 (m, 1H), |
| | 7.32 (br d, J = 8.0 Hz, 1H), 7.29-7.25 (m, 1H), 4.70-4.58 (m, 1H), 3.16 (br d, |
| | J = 7.5 Hz, 1H), 2.72 (br s, 1H), 2.23-2.05 (m, 5H), 2.02-1.92 (m, 2H), 1.92- |
| | 1.84 (m, 1H), 1.84-1.65 (m, 4H); NH and OH not observed |
| I-301 | MS m/z 408.5 [M + H]⁺; ¹H NMR (400 MHz, CD₃OD) δ 9.76 (s, 1H), 8.90 (s, |
| | 1H), 7.62-7.45 (m, 2H), 7.39-7.17 (m, 2H), 5.29 (d, J = 49.9 Hz, 1H), 5.07- |
| | 4.91 (m, 1H), 3.65-3.49 (m, 1H), 3.45-3.34 (m, 1H), 3.31-3.13 (m, 2H), 2.47- |
| | 2.29 (m, 1H), 2.29-2.04 (m, 1H). NH and OH not observed |
| I-302 | MS m/z 418.5 [M + H]⁺; ¹H NMR (400 MHz, CD₃OD) δ 9.62 (s, 1H), 8.86 (d, J = |
| | 5.9 Hz, 1H), 8.56 (s, 1H, formic acid), 7.56 (d, J = 7.9 Hz, 1H), 7.48 (d, J = |
| | 5.6 Hz, 1H), 7.33 (d, J = 8.0 Hz, 1H), 7.27 (s, 1H), 3.68 (d, J = 6.9 Hz, 2H), |
| | 3.51-3.37 (m, 2H), 2.86 (t, J = 12.5 Hz, 2H), 2.76 (s, 3H), 2.31-2.16 (m, |
| | 1H), 2.18-2.03 (m, 2H), 1.59 (q, J = 12.7 Hz, 2H). |
| I-303 | MS m/z 448.5 [M + H]⁺; ¹H NMR (400 MHz, CD₃OD) δ 9.63 (s, 1H), 8.86 (d, J = |
| | 5.7 Hz, 1H), 8.55 (s, 1H, formic acid), 7.57 (d, J = 7.8 Hz, 1H), 7.48 (d, J = |
| | 5.6 Hz, 1H), 7.32 (d, J = 7.9 Hz, 1H), 7.27 (s, 1H), 3.84 (t, J = 5.5 Hz, 2H), 3.68 |
| | (d, J = 6.9 Hz, 2H), 3.58-3.46 (m, 2H), 3.10 (t, J = 5.6 Hz, 2H), 2.86 (t, J = |
| | 12.5 Hz, 2H), 2.32-2.16 (m, 1H), 2.15-2.04 (m, 2H), 1.63 (q, J = 12.6 Hz, |
| | 2H). NH and OH not observed |
| I-305 | MS m/z 391.4 [M + H]⁺; ¹H NMR (400 MHz, CD₃OD) δ 9.70 (s, 1H), 8.94- |
| | 8.75 (m, 1H), 7.57 (d, J = 7.9 Hz, 1H), 7.47 (d, J = 5.4 Hz, 1H), 7.32 (d, J = 8.0 |
| | Hz, 1H), 7.26 (s, 1H), 4.35 (p, J = 8.0 Hz, 1H), 2.69 (t, J = 9.2 Hz, 2H), 2.27 (t, |
| | J = 9.7 Hz, 2H), 1.45 (s, 3H). NH and OH not observed |
| I-306 | MS m/z 408.4 [M + H]⁺; ¹H NMR (400 MHz, DMSO-d6) δ 10.51 (br s, 1 H), |
| | 9.78 (br s, 1 H), 8.85 (d, J = 5.63 Hz, 1 H), 8.17 (s, 1 H, formic acid), 7.68 (d, |
| | J = 7.25 Hz, 1 H), 7.56 (d, J = 7.63 Hz, 1 H), 7.35-7.26 (m, 3 H), 4.94 (d, |
| | J = 49.40 Hz, 1 H), 4.75-4.58 (m, 1 H), 3.24 (br s, 2 H), 3.12-2.98 (m, 1 H), |
| | 2.93-2.74 (m, 1 H), 2.70-2.57 (m, 1 H), 2.41-2.27 (m, 1 H), 2.12-1.88 (m, |
| | 1 H) |
| I-308 | MS m/z 419.3 [M + H]⁺; ¹H NMR (400 MHz, DMSO-d6) δ 10.48 (br s, 1H), |
| | 9.77 (s, 1H), 8.84 (d, J = 5.5 Hz, 1H), 7.79 (d, J = 7.5 Hz, 1H), 7.55 (d, J = 7.8 |
| | Hz, 1H), 7.37-7.22 (m, 3H), 4.45-4.29 (m, 1H), 3.30-3.23 (m, 5H), 2.12- |
| | 2.01 (m, 2H), 1.97-1.74 (m, 1H), 1.43-1.26 (m, 3H), 1.22-1.02 (m, 1H) |
| I-309 | MS m/z 391.3 [M + H]⁺; ¹H NMR (400 MHz, DMSO-d6) δ 10.48 (br s, 1H), |
| | 9.80 (s, 1H), 8.85 (d, J = 5.4 Hz, 1H), 7.68 (d, J = 7.4 Hz, 1H), 7.55 (d, J = 7.8 |
| | Hz, 1H), 7.34-7.26 (m, 3H), 4.52-4.35 (m, 1H), 4.15-4.04 (m, 1H), 3.89- |
| | 3.78 (m, 1H), 3.43-3.24 (m, 2H including H2O signal), 2.22-2.08 (m, 1H), |
| | 1.86-1.62 (m, 3H) |
| I-310 | MS m/z 391.3 [M + H]⁺; ¹H NMR (400 MHz, DMSO-d6) δ 10.50 (br s, 1H), |
| | 9.80 (s, 1H), 8.85 (d, J = 5.6 Hz, 1H), 7.68 (d, J = 7.3 Hz, 1H), 7.55 (d, J = 7.9 |
| | Hz, 1H), 7.35-7.25 (m, 3H), 4.53-4.36 (m, 1H), 4.16-4.02 (m, 1H), 3.87- |
| | 3.81 (m, 1H), 3.46-3.24 (m, 2H, including H2O signal), 2.22-2.09 (m, 1H), |
| | 1.86-1.60 (m, 3H) |
| I-315 | MS m/z 422.4 [M + H]⁺; ¹H NMR (400 MHz, CD₃OD) δ 9.77 (s, 1H), 8.86 (d, |
| | J = 5.7 Hz, 1H), 7.57 (d, J = 7.9 Hz, 1H), 7.49 (d, J = 5.6 Hz, 1H), 7.32 (d, J = |
| | 8.0 Hz, 1H), 7.26 (s, 1H), 5.15 (d, J = 50.5 Hz, 1H), 4.91 (d, J = 15.3 Hz, 1H), |
| | 3.11-3.00 (m, 1H), 2.74 (d, J = 11.7 Hz, 1H), 2.64 (t, J = 11.1 Hz, 1H), 2.50- |
| | 2.37 (m, 4H), 2.26-1.92 (m, 2H). |
| I-317 | MS m/z 448.4 [M + H]⁺; ¹H NMR (400 MHz, CD₃OD) δ 9.76 (s, 1H), 8.87 (s, |
| | 1H), 7.58 (d, J = 7.9 Hz, 1H), 7.48 (s, 1H), 7.32 (d, J = 7.9 Hz, 1H), 7.27 (s, |
| | 1H), 4.70-4.55 (m, 1H), 3.84-3.61 (m, 2H), 3.28-3.19 (m, 1H), 3.17-3.04 |
| | (m, 2H), 3.04-2.78 (m, 2H), 2.19-1.99 (m, 2H), 1.99-1.80 (m, 3H), 1.80- |
| | 1.61 (m, 1H). NH and OH not observed |

### Example 25

### Preparation of Compound I-313

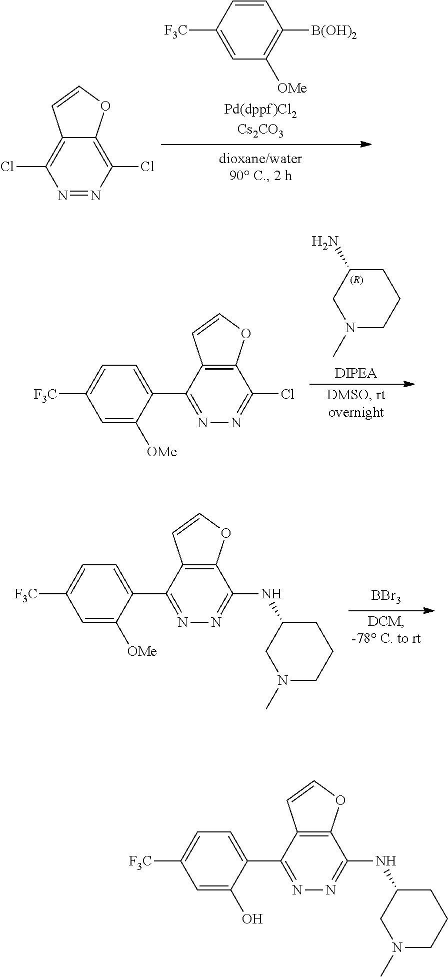

### Step 1. 7-Chloro-4-(2-methoxy-4-(trifluoromethyl)phenyl)furo[2,3-d]pyridazine

A mixture of 4,7-dichlorofuro[2,3-d]pyridazine (351 mg, 1.86 mmol, 1.0 eq.), [2-methoxy-4-(trifluoromethyl)phenyl]boronic acid (490 mg, 2.23 mmol, 1.2 eq.), [1,1′-bis(diphenylphosphino)ferrocence]dichloropalladium(II) (143 mg, 0.19 mmol, 0.1 eq.) and cesium carbonate (1.81 g, 5.57 mmol, 3 eq.) in water (0.33 M) and 1,4-dioxane (0.165 M) was heated at 90° C. for 2 hours. Upon completion, the reaction mixture was cooled to room temperature and diluted with EtOAc, washed with water and brine. The organic phase was dried over Na₂SO₄, filtered, and concentrated under reduced pressure. The crude residue was purified by silica gel column chromatography eluting with 0-50% EtOAc in hexanes to afford 7-chloro-4-(2-methoxy-4-(trifluoromethyl)phenyl)furo[2,3-d]pyridazine (495 mg). The impure isolation was used in next step without further purification. MS m/z 329.5, 330.8 [M+H]⁺.

### Step 2. (R)-4-(2-Methoxy-4-(trifluoromethyl)phenyl)-N-(1-methylpiperidin-3-yl)furo[2,3-d]pyridazin-7-amine

To a solution of the material from step 1 (417.8 mg, 1.271 mmol, 1 eq.) in DMSO (0.1 M) was added (3R)-1-methylpiperidin-3-amine (871 mg, 7.63 mmol, 6 eq.) and DIPEA (1.33 mL, 7.63 mmol, 6 eq.). The reaction was heated at 130° C. for 24 hours, then cooled to room temperature and diluted with EtOAc, washed with water, saturated NaHCO₃ (aq.), and brine. The organics were collected, dried over Na₂SO₄, and concentrated under reduced pressure. The crude was purified by silica gel column chromatography eluting with 0-70% MeOH (5% NH₃) in DCM to afford (R)-4-(2-methoxy-4-(trifluoromethyl)phenyl)-N-(1-methylpiperidin-3-yl)furo[2,3-d]pyridazin-7-amine as a brown solid (168.5 mg, 32%). MS m/z 407.8 [M+H]⁺; ¹H NMR (400 MHz, CD₃OD) δ 7.97 (d, J=1.8 Hz, 1H), 7.67 (d, J=8.0 Hz, 1H), 7.47-7.40 (m, 2H), 7.19 (s, 1H), 4.60-4.44 (m, 1H), 3.88 (s, 3H), 3.48-3.37 (m, 1H), 3.08-2.92 (m, 1H), 2.64-2.52 (m, 5H), 2.21-2.10 (m, 1H), 2.07-1.95 (m, 1H), 1.90-1.76 (m, 1H), 1.75-1.62 (m, 1H), 1 NH not observed.

### Step 3. (R)-2-(7-((1-Methylpiperidin-3-yl)amino)furo[2,3-d]pyridazin-4-yl)-5-(trifluoromethyl)phenol

At −78° C., to a solution of 4-[2-methoxy-4-(trifluoromethyl)phenyl]-N-[(3R)-1-methyl-3-piperidyl]furo[2,3-d]pyridazin-7-amine (100 mg, 0.25 mmol, 1 eq.) in DCM (0.2 M) was added boron tribromide (1 M in DCM) (2.2 ml, 2.21 mmol, 9 eq.). The reaction was kept at −78° C. for 30 min, then allowed to warm to room temperature and stirred for another 1 hour. Upon completion, the reaction was quenched by MeOH and was diluted with DCM. The mixture was vigorously stirred while saturate NaHCO₃ (aq.) was added slowly. The organic layer was washed with brine and concentrated in vacuo. The crude was purified by preparative reverse phase column (MeCN/water with 0.1% formic acid) to afford 2-[7-[[(3R)-1-methyl-3-piperidyl]amino]furo[2,3-d]pyridazin-4-yl]-5-(trifluoromethyl)phenol (41.4 mg, 43%) as a yellow solid. MS m/z 393.2 [M+H]⁺; ¹H NMR (400 MHz, CD₃OD) δ 8.32 (d, J=8.1 Hz, 1H), 7.98 (s, 1H), 7.11-7.00 (m, 3H), 4.40 (s, 1H), 3.65-3.44 (m, 1H), 3.19-3.05 (m, 1H), 2.86-2.44 (m, 5H), 2.14-1.93 (m, 2H), 1.88-1.78 (m, 1H), 1.71-1.58 (m, 1H), 1 OH and 1 NH not observed.

### Example 26

### Preparation of Compound I-163

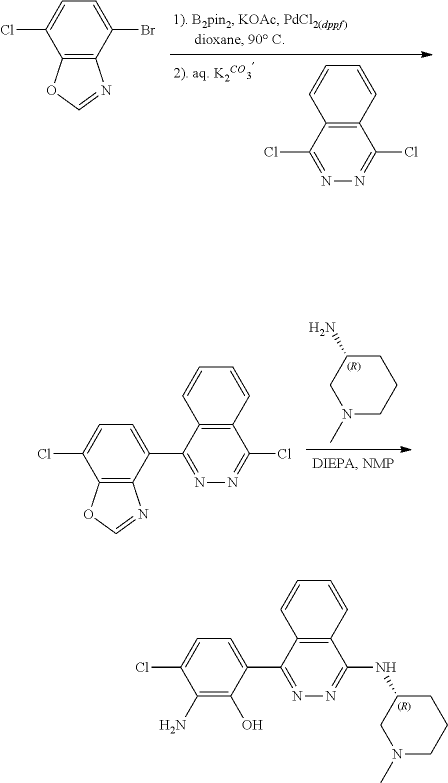

### Step 1. 7-Chloro-4-(4-chlorophthalazin-1-yl)benzo[d]oxazole

To a dry screw cap vial were added: 7-bromo-4-chloro-1,3-benzoxazole (60 mg, 0.26 mmol), 4,4,5,5-tetramethyl-2-(4,4,5,5-tetramethyl-1,3,2-dioxaborolan-2-yl)-1,3,2-dioxaborolane (72 mg, 0.28 mmol), KOAc (51 mg, 0.52 mmol), Pd(dppf)Cl₂ (19 mg, 0.03 mmol). The vial was purged with Argon and dioxane (2 ml) was added. The reaction mixture was heated to 90° C. for 2.5 h. UPLC showed complete consumption of the starting material. Cooled down to rt, used directly in the next step.

To the mixture above were added: 1,4-dichlorophthalazine (103 mg, 0.52 mmol), K₂CO₃ (72 mg, 0.52 mmol). The vial was purged with Argon for 15 min, and water (0.5 ml) was added. The reaction mixture was heated to 90° C. for 3 h. UPLC showed complete consumption of the starting material. The reaction was cooled and concentrated under reduced pressure. The residue was purified by flash column chromatography eluting with a gradient hexane/EtOAc (0-80% EtOAc) to obtain 4-chloro-7-(4-chlorophthalazin-1-yl)-1,3-benzoxazole (48 mg, 59% yield). MS m/z 315.7, 317.5 [M+H]⁺.

### Step 2. (R)-2-Amino-3-chloro-6-(4-((1-methylpiperidin-3-yl)amino)phthalazin-1-yl)phenol

To a solution of 4-chloro-7-(4-chlorophthalazin-1-yl)-1,3-benzoxazole (48 mg, 0.15 mmol) in NMP (1.0 mL) was added DIPEA (0.05 mL, 0.30 mmol) and the mixture was heated at 120° C. for 16 h. The reaction was cooled to rt, concentrated, the residue was purified by reverse phase chromatography eluting with a gradient ACN/H₂O/formic acid (0-100% ACN) to give (R)-2-amino-3-chloro-6-(4-((1-methylpiperidin-3-yl)amino)phthalazin-1-yl)phenol (7 mg, 12% yield) as a tan foam. MS m/z 384.1, 385.5 [M+H]⁺; ¹H NMR (CD₃OD) δ: 8.71 (d, J=8.3 Hz, 1H), 8.30 (td, J=7.8, 1.8 Hz, 1H), 8.15 (td, J=7.8, 0.8 Hz, 1H), 8.03 (br d, J=8.3 Hz, 1H), 7.14 (d, J=8.5 Hz, 1H), 6.76 (d, J=8.3 Hz, 1H), 4.65-4.75 (m, 1H), 3.88-3.98 (m, 1H), 3.61 (br d, J=11.5 Hz, 1H), 3.06-3.16 (m, 2H), 3.02 (s, 3H), 2.22-2.42 (m, 2H), 1.91-2.12 (m, 2H); 4 Hs not observed (3 NHs and OH).

### Example 27

### Preparation of Compounds I-30, I-44, I-96, and I-97

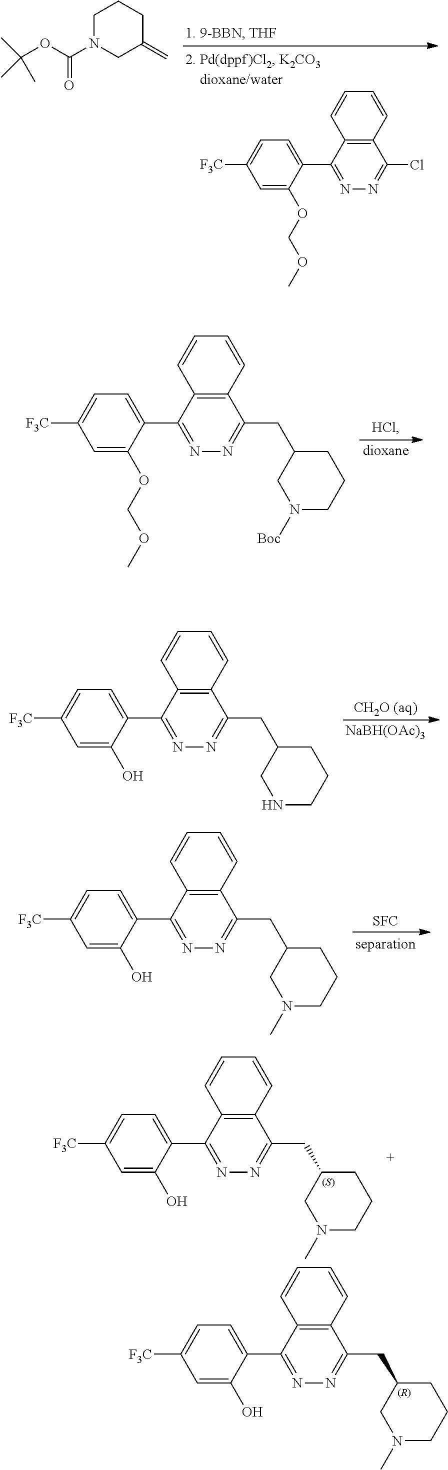

### Step 1. tert-Butyl 3-((4-(2-(methoxymethoxy)-4-(trifluoromethyl)phenyl)phthalazin-1-yl)methyl)piperidine-1-carboxylate

9-BBN (0.5 M in THF, 0.8 mL, 0.38 mmol, 1.5 eq.) was added to tert-butyl 3-methylenepiperidine-1-carboxylate (76 mg, 0.38 mmol, 1.5 eq.) under argon. The seal tube was heated at 65° C. for 2 hr, then this clear solution was added into a mixture of 1-chloro-4-[2-(methoxymethoxy)-4-(trifluoromethyl)phenyl]phthalazine (Intermediate 14b, 93.0 mg, 0.25 mmol, 1.0 eq.) and Pd(dppf)C₁₂ (16.6 mg, 0.020 mmol, 0.08 eq.) in K₂CO₃ (2M) in H₂O (0.38 mL, 3.0 eq.) and dioxane (2 mL) under argon. The sealed tube was heated to 90° C. o/n. After cooling, the reaction was diluted with EtOAc and NaHCO₃ (sat. aq.). The organic phase was washed with H₂O followed by brine. The organic phase was dried over MgSO₄, filtered, and concentrated in vacuo. The crude material was purified by flash column chromatography on silica gel eluting with 0-50% EtOAc in DCM to provide tert-butyl 3-[[4-[2-(methoxymethoxy)-4-(trifluoromethyl)phenyl]phthalazin-1-yl]methyl]piperidine-1-carboxylate (99 mg, 74% yield). MS m/z 532.2 [M+H]⁺;

### Step 2. 2-(4-(Piperidin-3-ylmethyl)phthalazin-1-yl)-5-(trifluoromethyl)phenol

Deprotection of tert-butyl 3-[[4-[2-(methoxymethoxy)-4-(trifluoromethyl)phenyl]phthalazin-1-yl]methyl]piperidine-1-carboxylate with HCl in dioxane, according to the procedure of Example 11, step 2, provided the title compound as hydrochloride salt. MS m/z 388.0 [M+H]⁺; ¹H NMR (CD₃OD) δ: 8.79 (d, J=8.3 Hz, 1H), 8.49 (t, J=7.6 Hz, 1H), 8.34 (t, J=7.7 Hz, 1H), 8.24 (d, J=8.3 Hz, 1H), 7.80 (br d, J=8.0 Hz, 1H), 7.51 (br d, J=7.9 Hz, 1H), 7.44 (s, 1H), 3.64-3.66 (m, 3H), 3.38-3.45 (m, 1H), 3.03 (br t, J=12.0 Hz, 2H), 2.65-2.75 (m, 1H), 1.99-2.13 (m, 2H), 1.82 (br d, J=13.6 Hz, 1H), 1.51-1.73 (m, 1H). NH and OH not observed.

### Step 3. 2-(4-((1-Methylpiperidin-3-yl)methyl)phthalazin-1-yl)-5-(trifluoromethyl)phenol

Reductive amination of 2-(4-(piperidin-3-ylmethyl)phthalazin-1-yl)-5-(trifluoromethyl)phenol with formaldehyde, according to the procedure of Example 12, step 3. The residue was then purified on prep-HPLC with 5-50% ACN in water with 0.1% formic acid to provide the title compound as formic acid salt. MS m/z 402.2 [M+H]⁺; ¹H NMR (CD₃OD) δ: 8.44 (s, 1H, formic acid), 8.28 (d, J=8.3 Hz, 1H), 7.97 (t, J=7.7 Hz, 1H), 7.86 (t, J=7.6 Hz, 1H), 7.70 (d, J=8.3 Hz, 1H), 7.48 (d, J=7.9 Hz, 1H), 7.25 (d, J=8.0 Hz, 1H), 7.19 (s, 1H), 3.23-3.39 (m, 2H), 3.11-3.17 (m, 1H), 3.02 (br d, J=10.6 Hz, 1H), 2.43 (s, 3H), 2.25-2.40 (m, 3H), 1.73-1.94 (m, 2H), 1.58 (br d, J=13.6 Hz, 1H), 1.17-1.31 (m, 1H). OH not observed.

### Step 4. (S)-2-(4-((1-methylpiperidin-3-yl)methyl)phthalazin-1-yl)-5-(trifluoromethyl)phenol and (R)-2-(4-((1-methylpiperidin-3-yl)methyl)phthalazin-1-yl)-5-(trifluoromethyl)phenol

The racemate from step 3 was separated by SFC purification to provide the following enantiomers (free base):

Compound I-96: MS m/z 402.2 [M+H]⁺; ¹H NMR (CD₃OD) δ: 8.41 (d, J=8.4 Hz, 1H), 8.08 (t, J=7.6 Hz, 1H), 7.98 (t, J=7.7 Hz, 1H), 7.82 (d, J=8.1 Hz, 1H), 7.59 (d, J=7.8 Hz, 1H), 7.36 (d, J=7.9 Hz, 1H), 7.30 (s, 1H), 3.35-3.46 (m, 2H), 3.08 (br d, J=11.3 Hz, 1H), 2.99 (br d, J=11.5 Hz, 1H), 2.35-2.45 (m, 4H), 2.14-2.31 (m, 2H), 1.77-1.96 (m, 2H), 1.58-1.71 (m, 1H), 1.26-1.37 (m, 1H). OH not observed.

Compound I-97: MS m/z 402.2 [M+H]⁺; ¹H NMR (CD₃OD) δ: 8.41 (d, J=8.4 Hz, 1H), 8.08 (t, J=7.6 Hz, 1H), 7.98 (t, J=7.7 Hz, 1H), 7.82 (d, J=8.3 Hz, 1H), 7.60 (d, J=7.8 Hz, 1H), 7.37 (d, J=8.0 Hz, 1H), 7.31 (s, 1H), 3.37-3.46 (m, 2H), 3.13 (br d, J=12.8 Hz, 1H), 3.03 (br d, J=10.8 Hz, 1H), 2.39-2.48 (m, 4H), 2.20-2.35 (m, 2H), 1.80-1.96 (m, 2H), 1.66 (br d, J=13.4 Hz, 1H), 1.15-1.38 (m, 1H). OH not observed.

### Example 28a

### Preparation of Compounds I-100 and I-118

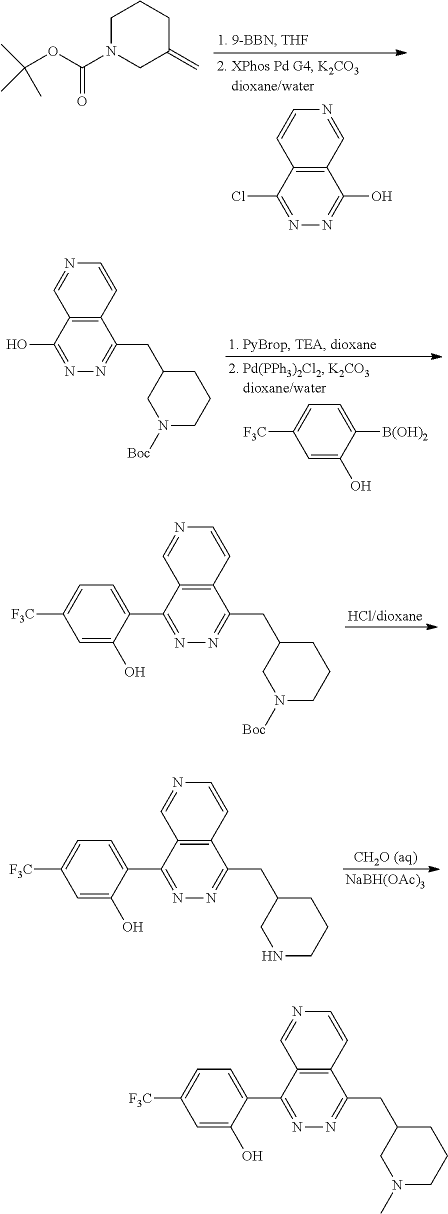

### Step 1. tert-Butyl 3-((4-hydroxypyrido[3,4-d]pyridazin-1-yl)methyl)piperidine-1-carboxylate

tert-Butyl 3-methylenepiperidine-1-carboxylate (318 mg, 1.61 mmol, 1.5 eq.) in 9-BBN (0.5 M in THF, 3.0 mL, 1.5 eq.) was heated at 60° C. for 2 hr. After cooled to rt, it was added to a mixture of 1-chloro-3H-pyrido[3,4-d]pyridazin-4-one (Intermediate 1a, 195 mg, 1.07 mmol, 1.0 eq.) and XPhos Pd G4 (94 mg, 0.10 eq.) in K₂CO₃ (2M) in H₂O (1.6 mL, 3.0 eq.) and dioxane (4 mL) under argon. The sealed tube was heated at 100° C. for 4 hr. After cooled to rt, the reaction was diluted with EtOAc, water with a few drop of citric acid (1.0M). The organic phase was washed with H₂O followed by brine. The organic phase was dried over MgSO₄, filtered, and concentrated in vacuo. The crude material was purified by flash column chromatography on silica gel eluting with 0-30% MeOH in DCM to provide tert-butyl 3-[(4-oxo-3H-pyrido[3,4-d]pyridazin-1-yl)methyl]piperidine-1-carboxylate (320 mg, 86% yield). MS m/z 345.2 [M+H]⁺;

### Step 2. tert-Butyl 3-[[4-[2-hydroxy-4-(trifluoromethyl)phenyl]pyrido[3,4-d]pyridazin-1-yl]methyl]piperidine-1-carboxylate

A mixture of tert-butyl 3-[(4-oxo-3H-pyrido[3,4-d]pyridazin-1-yl)methyl]piperidine-1-carboxylate (72 mg, 0.20 mmol, 1.0 eq.) and PyBrOP (150 mg, 0.31 mmol, 1.5 eq.) in Et₃N (64 mg, 3.0 eq.) and dioxane (2 mL) was heated at 100° C. for 2 hr. After cooled to rt, [2-hydroxy-4-(trifluoromethyl)phenyl]boronic acid (86 mg, 2.0 eq.), Pd(PPh₃)₂Cl₂ (14 mg, 0.1 eq.) and K₂CO₃ (88 mg, 3.0 eq.) was added under argon, followed by addition of H₂O (0.2 mL). The sealed tube was heated at 100° C. for 12 hr. After cooled to rt, the reaction was diluted with EtOAc, water with a few drops of citric acid (1.0M). The organic phase was washed with H₂O followed by brine. The organic phase was dried over MgSO₄, filtered, and concentrated in vacuo. The crude material was purified by flash column chromatography on silica gel eluting with 0-30% MeOH in DCM to provide a mixture of unreacted starting material and tert-butyl 3-[[4-[2-hydroxy-4-(trifluoromethyl)phenyl]pyrido[3,4-d]pyridazin-1-yl]methyl]piperidine-1-carboxylate (35 mg, 34% yield), which was then further purified on pre-HPLC, eluting with 20-90% ACN in water (with 0.1% FA).

### Step 3. 2-(4-(Piperidin-3-ylmethyl)pyrido[3,4-d]pyridazin-1-yl)-5-(trifluoromethyl)phenol hydrochloride

Deprotection tert-butyl 3-[[4-[2-hydroxy-4-(trifluoromethyl)phenyl]pyrido[3,4-d]pyridazin-1-yl]methyl]piperidine-1-carboxylate with HCl in dioxane provided the title compound as hydrochloride salt. MS m/z 389.1 [M+H]⁺; ¹H NMR (CD₃OD) δ: 9.41 (s, 1H), 9.25 (br d, J=5.6 Hz, 1H), 8.40 (br d, J=5.6 Hz, 1H), 7.79 (d, J=7.8 Hz, 1H), 7.48 (d, J=8.0 Hz, 1H), 7.40 (s, 1H), 3.54-3.66 (m, 3H), 3.37-3.45 (m, 1H), 2.94-3.06 (m, 2H), 2.64-2.75 (m, 1H), 1.99-2.14 (m, 2H), 1.82 (br d, J=13.9 Hz, 1H), 1.53-1.66 (m, 1H). NH and OH not observed.

### Step 4. 2-(1-((1-Methylpiperidin-3-yl)methyl)pyrido[3,4-d]pyridazin-4-yl)-5-(trifluoromethyl)phenol

Reductive amination of 2-(1-(piperidin-3-ylmethyl)pyrido[3,4-d]pyridazin-4-yl)-5-(trifluoromethyl)phenol with formaldehyde, according to the procedure of Example 12, step 3. The residue was then purified on prep-HPLC with 5-50% ACN in water with 0.1% formic acid to provide the title compound as formic acid salt. MS m/z 403.2 [M+H]⁺; ¹H NMR (CD₃OD) δ: 9.10 (s, 1H), 8.96 (d, J=5.8 Hz, 1H), 8.37 (s, 1H, formic acid), 8.12 (d, J=5.8 Hz, 1H), 7.58 (d, J=7.8 Hz, 1H), 7.30 (d, J=8.1 Hz, 1H), 7.21 (s, 1H), 3.36-3.51 (m, 2H), 3.23-3.35 (m, 2H), 2.63-2.75 (m, 5H), 2.50-2.61 (m, 1H), 1.84-1.95 (m, 2H), 1.68 (br d, J=14.3 Hz, 1H), 1.29-1.40 (m, 1H). OH not observed.

### Example 28b

### Preparation of Compound I-567

The compound below was prepared in analogous manner according to the procedure of Example 28a, using 4-bromopyrido[3,4-d]pyridazin-1-ol (Intermediate 1b) in place of 1-chloro-3H-pyrido[3,4-d]pyridazin-4-one in step 1.

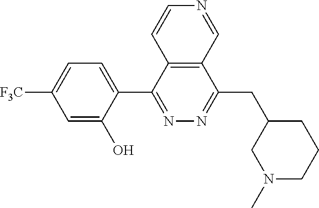 [SMILES: CN1CCCC(Cc2nnc(-c3ccc(C(F)(F)F)cc3O)c3ccncc23)C1]

MS m/z 403.2 [M+H]⁺; ¹H NMR (CD₃OD) δ: 9.71 (s, 1H), 8.88 (d, J=5.6 Hz, 1H), 7.59 (d, J=5.8 Hz, 1H), 7.51 (d, J=7.9 Hz, 1H), 7.22 (d, J=8.3 Hz, 1H), 7.18 (s, 1H), 3.38 (d, J=7.1 Hz, 2H), 3.12-3.20 (m, 1H), 2.90 (br d, J=9.0 Hz, 1H), 2.68-2.85 (m, 1H), 2.18-2.32 (m, 4H), 1.93-2.09 (m, 2H), 1.82-1.92 (m, 1H), 1.61-1.82 (m, 1H), 1.43-1.59 (m, 1H). OH not observed.

### Example 29

### Preparation of Compounds I-255 and I-256

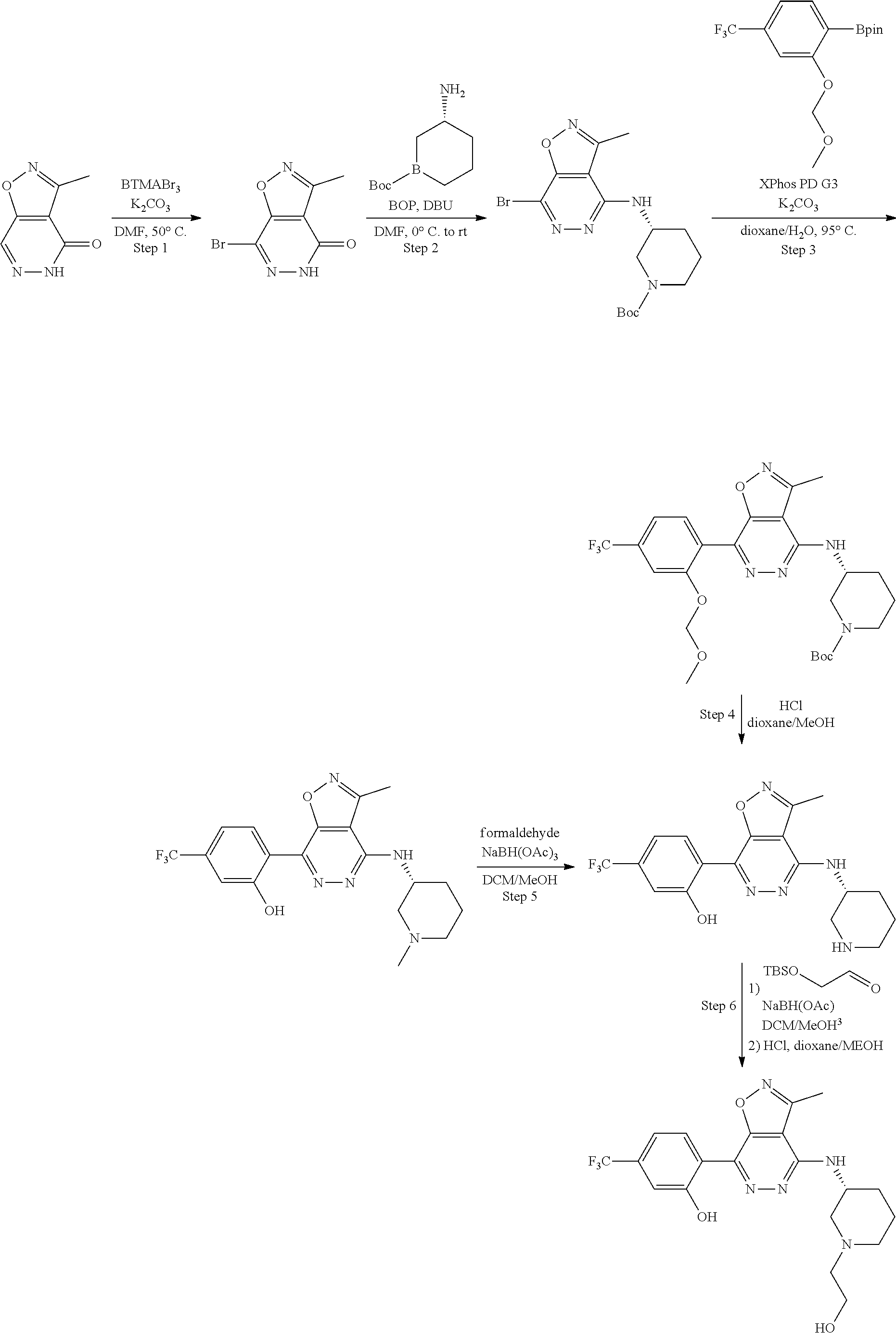

### Step 1: 7-Bromo-3-methyl-5H-isoxazolo[4,5-d]pyridazin-4-one

A suspension of 3-methyl-5H-isoxazolo[4,5-d]pyridazin-4-one (prepared according to U.S. patent application publication number US2017/73353) (1.45 g, 9.59 mmol) in DMF (21 mL) was added K₂CO₃ (2.65 g, 19.2 mmol), and the mixture was stirred for 10 min at rt. Benzyl trimethylammonium tribromide (7.71 g, 19.2 mmol) was added, and the mixture was heated to 50° C. for 2 h. After cooling to rt, the mixture was filtered through celite, washing with EtOAc. The filtrate was washed with water (2×10 mL). The aqueous phase was extracted with EtOAc, and the combined organic extracts were washed with sat. Na₂S₂O₃, brine, dried (Mg₂SO₄), filtered and concentrated. Purification by chromatography on SiO₂ (EtOAc:hexanes, 15-60%) gave a pale yellow solid (1.40 g, 63%%). MS m/z 229.9, 231.9 [M+H]⁺.

### Step 2. t-Butyl (3R)-3-[(7-bromo-3-methyl-isoxazolo[4,5-d]pyridazin-4-yl)amino]piperidine-1-carboxylate

To a mixture of 7-bromo-3-methyl-5H-isoxazolo[4,5-d]pyridazin-4-one (0.250 g, 1.09 mmol) and t-butyl (3R)-3-aminopiperidine-1-carboxylate (0.653 g, 3.26 mmol) in DMF (4.3 mL) was added DBU (0.34 mL, 2.23 mmol). The mixture was cooled to 0° C. and was added BOP (0.520 g, 1.14 mmol). The reaction mixture was stirred at 0° C. for 5 min before warming to rt. The reaction was stirred 14 h before diluting with DCM/iPrOH (9:1). The mixture was washed with sat. NaHCO₃, H₂O, and brine. The organics were dried (Na₂SO₄), filtered and concentrated. Purification by chromatography on SiO₂ (EtOAc:hexanes, 0 to 65%) gave a yellow foam (0.204 g, 46%). MS m/z 411.9, 413.8 [M+H]⁺.

### Step 3: t-butyl (3R)-3-[[7-[2-(methoxymethoxy)-4-(trifluoromethyl)phenyl]-3-methyl-isoxazolo[4,5-d]pyridazin-4-yl]amino]piperidine-1-carboxylate

t-Butyl (3R)-3-[(7-bromo-3-methyl-isoxazolo[4,5-d]pyridazin-4-yl)amino]piperidine-1-carboxylate (0.210 g, 0.509 mmol), 2-[2-(methoxymethoxy)-4-(trifluoromethyl)phenyl]-4,4,5,5-tetramethyl-1,3,2-dioxaborolane (0.271 g, 0.815 mmol), and XPhos Pd G3 (0.035 g, 0.041 mmol) were added to a vial and evacuated and refilled with Ar. The mixture was dissolved in dioxane (2.5 mL) and K₂CO₃ (2 M, 0.76 mL, 1.53 mmol). The mixture was sparged with Ar for 5 min. The reaction was then heated to 95° C. for 24 h. The mixture was diluted with EtOAc and filtered through Celite. The filtrate was washed with brine and concentrated. Purification by chromatography on SiO₂ (EtOAc:hexanes, 0 to 65%) gave a yellow foam (0.106 g, 39%). MS m/z 538.5 [M+H]⁺.

### Step 4: 2-[3-methyl-4-[[(3R)-3-piperidyl]amino]isoxazolo[4,5-d]pyridazin-7-yl]-5-(trifluoromethyl)phenol

t-Butyl (3R)-3-[[7-[2-(methoxymethoxy)-4-(trifluoromethyl)phenyl]-3-methyl-isoxazolo[4,5-d]pyridazin-4-yl]amino]piperidine-1-carboxylate (0.130 g, 0.242 mmol) was dissolved in dioxane (0.5 mL) and added HCl (4M in dioxane, 1.5 mL). The reaction was stirred at rt for 1.5 h. The reaction was diluted with DCM/iPrOH(9:1) and washed with NaHCO₃, H₂O, and brine. The organics were dried (Na₂SO₄), filtered and concentrated to give a yellow foam (0.095 g, quant.). MS m/z 394.4 [M+H]⁺.

### Step 5: 2-[3-methyl-4-[[(3R)-1-methyl-3-piperidyl]amino]isoxazolo[4,5-d]pyridazin-7-yl]-5-(trifluoromethyl)phenol formic acid salt

2-[3-Methyl-4-[[(3R)-3-piperidyl]amino]isoxazolo[4,5-d]pyridazin-7-yl]-5-(trifluoromethyl)-phenol (0.045 g, 0.11 mmol) in DCM (1.1 mL) was added a methanolic formaldehyde solution (formaldehyde 37% in water, 28 μL, in MeOH 28 μL) followed by NaBH(OAc)₃ (0.061 g, 0.23 mmol). The reaction was stirred for 20 min before dilution with DCM. The mixture was then washed with sat. NaHCO₃, water, and brine. The organic phase was dried (Na₂SO₄), filtered, and concentrated. Purification by reverse phase chromatography (0.1% formic acid in MeCN:0.1% formic acid in H₂O, 5 to 100%) gave the title compound as formic acid salt (0.022 g, 42%). MS m/z 408.4 [M+H]⁺; H NMR (400 MHz, DMSO-d₆) δ 13.28 (s, 1H), 8.25 (d, J=8.1 Hz, 1H), 8.18 (s, 1H, formic acid), 7.39 (d, J=8.1 Hz, 1H), 7.32 (s, 1H), 6.67 (d, J=7.9 Hz, 1H), 4.51-4.38 (m, 1H), 2.90 (d, J=9.5 Hz, 1H), 2.76 (s, 3H), 2.63-2.54 (m, 1H), 2.23 (s, 3H), 2.20-2.12 (m, 1H), 2.11-2.02 (m, 1H), 1.96-1.84 (m, 1H), 1.81-1.70 (m, 1H), 1.68-1.51 (m, 2H)

### Step 6: 2-[3-methyl-4-[[(3R)-1-(2-hydroxyethyl)-3-piperidyl]amino]isoxazolo[4,5-d]pyridazin-7-yl]-5-(trifluoromethyl)phenol formic acid salt

2-[3-Methyl-4-[[(3R)-3-piperidyl]amino]isoxazolo[4,5-d]pyridazin-7-yl]-5-(trifluoromethyl)-phenol (0.045 g, 0.11 mmol) in DCM (1.1 mL) was added a 2-[t-butyl-(dimethyl)silyl]oxyacetaldehyde solution (0.060 g, in MeOH 27 μL) followed by NaBH(OAc)₃ (0.061 g, 0.23 mmol). The reaction was stirred for 20 min before dilution with DCM. The mixture was then washed with sat. NaHCO₃, water, and brine. The organic phase was dried (Na₂SO₄), filtered, and concentrated to give a yellow oil. The oil was then dissolved in dioxane (0.5 mL) and added HCl (4M in dioxane, 1.0 mL). The mixture was stirred at rt for 15 min. The reaction was concentrated and redissolved in DCM. The solution was washed with sat. NaHCO₃, brine, dried (Na₂SO₄), filtered and concentrated. Purification by reverse phase chromatography (0.1% formic acid in MeCN:0.1% formic acid in H₂O, 5 to 100%) gave a yellow solid (0.024 g, 43%). MS m/z 438.4 [M+H]⁺; ¹H NMR (400 MHz, DMSO-d₆) δ 8.24 (d, J=8.00 Hz, 2H, with formic acid), 7.39 (d, J=8.25 Hz, 1H), 7.32 (s, 1H), 6.73-6.61 (m, 1H), 4.51-4.42 (m, 1H), 3.53 (t, J=6.19 Hz, 2H), 2.94-2.85 (m, 1H), 2.76 (s, 3H), 2.66-2.55 (m, 1H), 2.48-2.36 (m, 3H), 2.35-2.01 (m, 3H), 1.95-1.52 (m, 3H). 3 exchangeable protons not observed.

### Example 30

### Preparation of Compounds I-144 and I-146

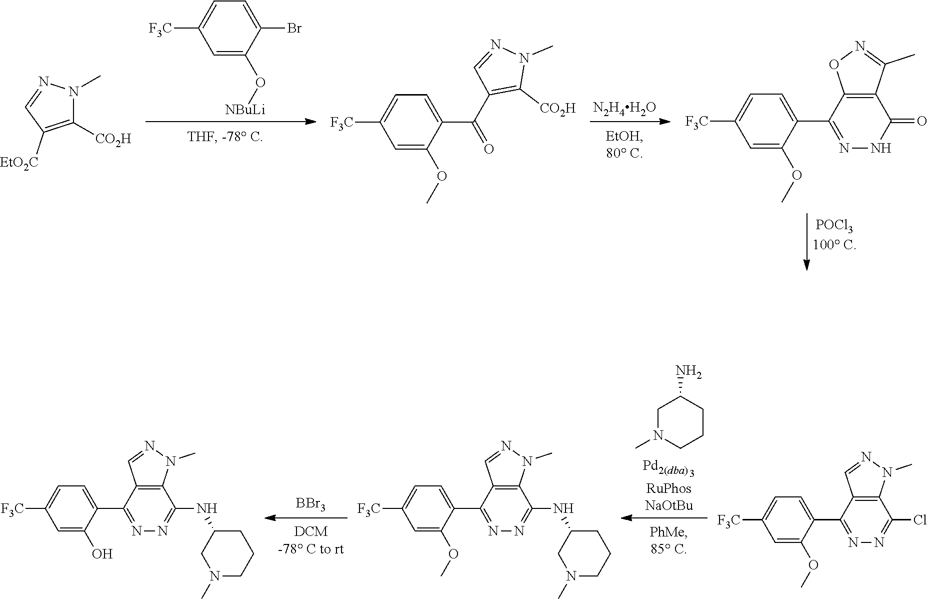

### Step 1. 4-[2-Methoxy-4-(trifluoromethyl)benzoyl]-2-methyl-pyrazole-3-carboxylic acid

A solution of 1-bromo-2-methoxy-4-(trifluoromethyl)benzene (1.22 g, 4.79 mmol) in THF (5 mL) was cooled to −78° C. was added nBuLi (2.5 M in hexanes, 1.90 mL, 4.79 mmol) and stirred for 40 min. Separately, 4-ethoxycarbonyl-2-methyl-pyrazole-3-carboxylic acid (prepared according to WO2014/27009, 2014, A1) (0.500 g, 2.52 mmol) in THF (16 mL) at −78° C. was added the aryl lithium solution by cannula over ca. 2 min. The reaction was stirred for 5 min before the addition of sat. NH₄Cl at −78° C. The resulting heterogenous mixture was warmed to rt. The quenched reaction was partitioned between EtOAc/water. The aqueous phase was extracted with EtOAc. The combined organic extracts were washed with brine, dried (MgSO₄), filtered, and concentrated. Purification by chromatography on SiO₂ (EtOAc:hexanes 10% to 1% AcOH in EtOAc) followed by reverse phase chromatography (MeCN:H₂O, 5 to 100%) gave a white solid (0.127 g, 15%). MS m/z 329.0 [M+H]⁺; ¹H NMR (400 MHz, DMSO-d₆) δ 14.16 (br s, 1H), 7.75 (s, 1H), 7.55 (d, J=7.78 Hz, 1H), 7.41 (s, 1H), 7.40-7.37 (m, 1H), 3.97 (s, 3H), 3.78 (s, 3H).

### Step 2. 4-[2-Methoxy-4-(trifluoromethyl)phenyl]-1-methyl-6H-pyrazolo[3,4-d]pyridazin-7-one

A mixture of 4-[2-methoxy-4-(trifluoromethyl)benzoyl]-2-methyl-pyrazole-3-carboxylic acid (0.278 g, 0.847 mmol) in EtOH (3.5 mL) was added N₂H₄H₂O (0.12 mL, 2.54 mL) and 2 drops of acetic acid. The reaction was stirred at 80° C. for 1 h and then cooled in ice-bath. The heterogeneous mixture was filtered and washed with cold EtOH to give a white solid (0.225 g, 82%). MS m/z 324.8 [M+H]⁺.

### Step 3: 7-Chloro-4-[2-methoxy-4-(trifluoromethyl)phenyl]-1-methyl-pyrazolo[3,4-d]pyridazine

POCl₃ (0.85 mL) was added to 4-[2-methoxy-4-(trifluoromethyl)phenyl]-1-methyl-6H-pyrazolo[3,4-d]pyridazin-7-one (0.082 g, 0.250 mmol) and heated to 100° C. for 45 min. The reaction was cooled to rt and concentrated to give a pale yellow solid. The solid was dissolved in DCM/MeOH (10:1) and washed with sat. NaHCO₃ and brine. The organic phase was dried (Na₂SO₄), filtered, and concentrated. Purification by chromatography (DCM/MeOH, 0 to 10%) gave a white solid (0.052 g, 60%). MS m/z 343.0, 344.8 [M+H]⁺.

### Step 4: 4-[2-Methoxy-4-(trifluoromethyl)phenyl]-1-methyl-N-[(3R)-1-methyl-3-piperidyl]pyrazolo[3,4-d]pyridazin-7-amine formic acid salt

7-Chloro-4-[2-methoxy-4-(trifluoromethyl)phenyl]-1-methyl-pyrazolo[3,4-d]pyridazine (0.070 g, 0.200 mmol), Pd₂(dba)₃ (0.019 g, 0.020 mol), RuPhos (0.017 g, 0.041 mmol), NaOt-Bu (0.039 g, 0.41 mmol), and (3R)-1-methylpiperidin-3-amine (0.028 g, 0.25 mmol) in PhMe (1.0 mL) was heated to 85° C. for 1.5 h. The reaction was diluted with EtOAc and added sat. NH₄Cl. The layers were separated, and the aqueous phase extracted with EtOAc. The combined organic extracts were washed with brine, dried (Na₂SO₄), filtered, and concentrated. Purification by chromatography on SiO₂ (1M NH₄OH in MeOH:DCM, 0 to 10%) followed by reverse phase (0.1% formic acid in MeCN:0.1% formic acid in H₂O, 5 to 100%) gave a tan solid (0.055 g, 64%). MS m/z 421.1 [M+H]⁺; ¹H NMR (400 MHz, DMSO-d₆) δ 8.18 (s, 1H, formic acid), 7.87 (s, 1H), 7.69 (d, J=7.6 Hz, 1H), 7.51-7.40 (m, 2H), 6.25 (d, J=6.9 Hz, 1H), 4.44-4.36 (m, 1H), 4.33 (s, 3H), 3.85 (s, 3H), 3.05-2.96 (m, 1H), 2.70-2.58 (m, 1H), 2.27 (s, 3H), 2.23-2.09 (m, 2H), 1.94 (br s, 1H), 1.85-1.69 (m, 1H), 1.69-1.44 (m, 2H)

### Step 5: 2-[1-Methyl-7-[[(3R)-1-methyl-3-piperidyl]amino]pyrazolo[3,4-d]pyridazin-4-yl]-5-(trifluoromethyl)phenol formic acid salt

A solution of 4-[2-methoxy-4-(trifluoromethyl)phenyl]-1-methyl-N-[(3R)-1-methyl-3-piperidyl]pyrazolo[3,4-d]pyridazin-7-amine (0.055 g, 0.13 mmol) in DCM (0.5 mL) was cooled to −78° C. and added BBr₃ (1.0 M in DCM, 1.3 ml, 1.3 mmol). The reaction was stirred at −78° C. for 30 min and let warm to rt, followed by stirring for 1.5 h. The reaction was quenched with MeOH (0.5 mL) and diluted with DCM (20 mL), stirring 10 min. Sat. NaHCO₃ (1.0 mL) was added and vigorously stirred for 15 min. The layers were separated, and the aqueous phase was extracted with DCM. The combined organic extracts were dried (Na₂SO₄), filtered and concentrated. Purification by chromatography on SiO₂ (1M NH₄OH in MeOH:DCM, 0 to 10%) followed by reverse phase chromatography (0.1% formic acid in MeCN:0.1% formic acid in H₂O, 5 to 100%) gave a yellow solid (0.014 g, 24%). MS m/z 407.2 [M+H]⁺; ¹H NMR (400 MHz, DMSO-d₆) δ 8.69 (s, 1H), 8.28 (d, J=8.0 Hz, 1H), 8.18 (s, 1H, formic acid), 7.31-7.21 (m, 2H), 6.56 (d, J=7.5 Hz, 1H), 4.44-4.30 (m, 4H), 3.02-2.90 (m, 1H), 2.65-2.54 (m, 1H), 2.30-2.16 (m, 4H), 2.17-2.06 (m, 1H), 1.98-1.87 (m, 1H), 1.84-1.70 (m, 1H), 1.67-1.51 (m, 2H). 1H not observed (OH).

### Example 31

### Preparation of Compounds I-104 and I-321

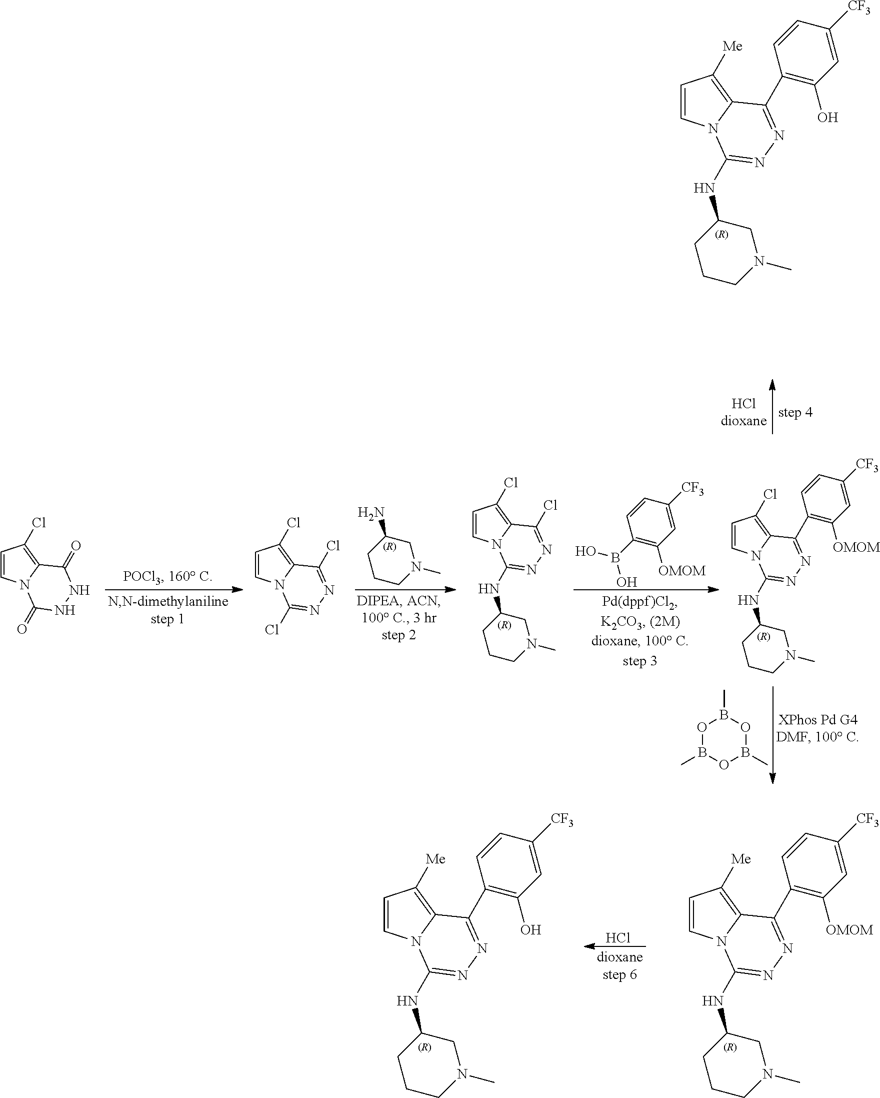

### Step 1. 1,4,8-Trichloropyrrolo[1,2-d][1,2,4]triazine

To a stirred solution of 8-chloro-2,3-dihydropyrrolo[1,2-d][1,2,4]triazine-1,4-dione (Intermediate 2c, 2.80 g, 13.7 mmol) in POCl₃ (28 mL) was added N,N-dimethylaniline (1.66 g, 13.7 mmol). The reaction mixture was heated at 120° C. for 16 h and then evaporated under reduced pressure to give a residue. The residue was diluted with cold saturated sodium bicarbonate solution (100 mL) and extracted with dichloromethane (3×50 mL). The combined organic extracts were dried over sodium sulfate and evaporated under reduced 5 pressure to give a residue which was purified by silica gel column chromatography eluting with EtOAc/hexanes to yield 1,4,8-trichloropyrrolo[1,2-d][1,2,4]triazine (340 mg, 10%). MS m/z 221.9, 223.9 [M+H]⁺; ¹H NMR (400 MHz, DMSO-d6) δ: 8.10 (d, J=4.0 Hz, 1H), 7.36 (d, J=4.0 Hz, 1H).

### Step 2. 1,8-Dichloro-N-[(3R)-1-methyl-3-piperidyl]pyrrolo[1,2-d][1,2,4]triazin-4-amine

To a solution of (3R)-1-methylpiperidin-3-amine dihydrochloride (330 mg, 1.76 mmol) in acetonitrile (5 mL) was added 1,4,8-trichloropyrrolo[1,2-d][1,2,4]triazine (250 mg, 1.12 mmol) and N,N-diisopropylethylamine (520 mg, 4.02 mmol). The mixture was stirred at 100° C. for 3 h. LCMS showed the reaction was completed. The mixture was concentrated to give residue which was purified by silica column (DCM/MeOH=94/6) to give 1,8-dichloro-N-[(3R)-1-methyl-3-piperidyl]pyrrolo[1,2-d][1,2,4]triazin-4-amine (155 mg, 46% Yield). MS m/z 300.1 [M+H]⁺.

### Step 3. 8-Chloro-1-[2-(methoxymethoxy)-4-(trifluoromethyl)phenyl]-N-[(3R)-1-methyl-3-piperidyl]pyrrolo[1,2-d][1,2,4]triazin-4-amine

To a solution of [2-(methoxymethoxy)-4-(trifluoromethyl)phenyl]boronic acid (560 mg, 2.24 mmol), potassium carbonate (450 mg, 3.26 mmol), 1,8-dichloro-N-[(3R)-1-methyl-3-piperidyl]pyrrolo[1,2-d][1,2,4]triazin-4-amine (225 mg, 0.75 mmol) and [1,1′-bis(diphenylphosphino)ferrocene] dichloropalladium(ii) (110 mg, 0.14 mmol) in 1,4-dioxane (10 mL) and water (2 mL) was stirred at 100° C. for 16 hours under N₂. The mixture was cooled to r.t. H₂O was added and the mixture was extracted with EtOAc (100 mL×3) and combined organic phases were washed with brine, dried over sodium sulfate, filtered, and concentrated to give residue which was purified by column chromatography (MeOH/DCM=5/95) to afford 8-chloro-1-[2-(methoxymethoxy)-4-(trifluoromethyl)phenyl]-N-[(3R)-1-methyl-3-piperidyl]pyrrolo[1,2-d][1,2,4]triazin-4-amine (245 mg, 69% Yield) as a yellow solid. MS m/z 470.2 [M+H]⁺.

### Step 4. 2-[8-Chloro-4-[[(3R)-1-methyl-3-piperidyl]amino]pyrrolo[1,2-d][1,2,4]triazin-1-yl]-5-(trifluoromethyl)phenol

8-Chloro-1-[2-(methoxymethoxy)-4-(trifluoromethyl)phenyl]-N-[(3R)-1-methyl-3-piperidyl]pyrrolo[1,2-d][1,2,4]triazin-4-amine (70 mg, 0.15 mmol) in dichloromethane (2 mL) was added hydrochloric acid in dioxane (2 mL, 8 mmol, 4 mol/L) at rt. The reaction mixture was stirred at r.t. for 2 h. The mixture was concentrated. The mixture was added Sat. NaHCO₃ and stirred for 10 min. The mixture was extracted with EtOAc (50 ml×3). The combined organic layers was washed with brined, dried and concentrated to give residue which was purified by silica column (DCM/MeOH=95/5) to give 2-[8-chloro-4-[[(3R)-1-methyl-3-piperidyl]amino]pyrrolo[1,2-d][1,2,4]triazin-1-yl]-5-(trifluoromethyl)phenol (50 mg, 79% Yield) as a solid. MS m/z 426.0 [M+H]⁺. ¹H NMR (400 MHz, CD₃OD) δ 7.83 (s, 1H), 7.44 (d, J=8.0 Hz, 1H), 7.23 (d, J=8.0 Hz, 1H), 7.16 (s, 1H), 6.91 (s, 1H), 4.50-4.41 (m, 1H), 3.22-3.18 (m, 1H), 2.86-2.82 (m, 1H), 2.44-2.31 (m, 5H), 2.12-2.09 (m, 1H), 1.94-1.90 (m, 1H), 1.79-1.75 (m, 1H), 1.66-1.63 (m, 1H), NH and OH not observed.

### Step 5. (R)-1-(2-(Methoxymethoxy)-4-(trifluoromethyl)phenyl)-8-methyl-N-(1-methylpiperidin-3-yl)pyrrolo[1,2-d][1,2,4]triazin-4-amine

A solution of 2,4,6-trimethyl-1,3,5,2,4,6-trioxatriborinane (300 mg, 2.39 mmol), potassium carbonate (120 mg, 0.87 mmol), 8-chloro-1-[2-(methoxymethoxy)-4-(trifluoromethyl)phenyl]-N-[(3R)-1-methyl-3-piperidyl]pyrrolo[1,2-d][1,2,4]triazin-4-amine (180 mg, 0.38 mmol) and XPhos Pd G4 (50 mg, 0.057 mmol) in N,N-dimethylformamide (4 mL) and water (0.4 mL) was stirred at 100° C. for 12 hours under N₂. Water was added and the mixture was extracted with EtOAc (100 mL×3). The combined organic layers was washed by H₂O and brine, then dried and concentrated under vacuum to give residue which was purified by silica column (DCM/MeOH=95/5) to give the mixture (39 mg) of the titled compound and de-C₁ of starting material, which was further purified by SFC separation to provide (R)-1-(2-(methoxymethoxy)-4-(trifluoromethyl)phenyl)-8-methyl-N-(1-methylpiperidin-3-yl)pyrrolo[1,2-d][1,2,4]triazin-4-amine (5 mg) as a yellow solid. MS m/z 450.0 [M+H]⁺.

### Step 6. (R)-2-(8-Methyl-4-((1-methylpiperidin-3-yl)amino)pyrrolo[1,2-d][1,2,4]triazin-1-yl)-5-(trifluoromethyl)phenol

(R)-1-(2-(Methoxymethoxy)-4-(trifluoromethyl)phenyl)-8-methyl-N-(1-methylpiperidin-3-yl)pyrrolo[1,2-d][1,2,4]triazin-4-amine (5 mg) in was stirred in hydrochloric acid in dioxane (4M in dioxane, 0.2 mL) at room temperature for 1 h. The mixture was concentrated, to which Sat. NaHCO₃ was added and stirred for 10 min. The mixture was extracted with EtOAc. The combined organic layers were washed with brined, dried and concentrated to give residue which was purified by silica column, eluting with DCM/MeOH to give the title compound (2.5 mg). MS m/z 406.2 [M+H]⁺; ¹H NMR (CD₃OD) δ: 7.73 (d, J=2.9 Hz, 1H), 7.45 (d, J=7.9 Hz, 1H), 7.27 (d, J=7.9 Hz, 1H), 7.20 (s, 1H), 6.74-6.78 (m, 1H), 4.39-4.51 (m, 1H), 3.09-3.21 (m, 1H), 2.69-2.81 (m, 1H), 2.37 (s, 3H), 2.29 (br dd, J=4.8, 3.3 Hz, 2H), 2.02-2.18 (m, 1H), 1.84-1.95 (m, 4H), 1.70-1.81 (m, 1H), 1.59-1.68 (m, 1H), NH and OH not observed

### Example 32

### Preparation of Compound I-361

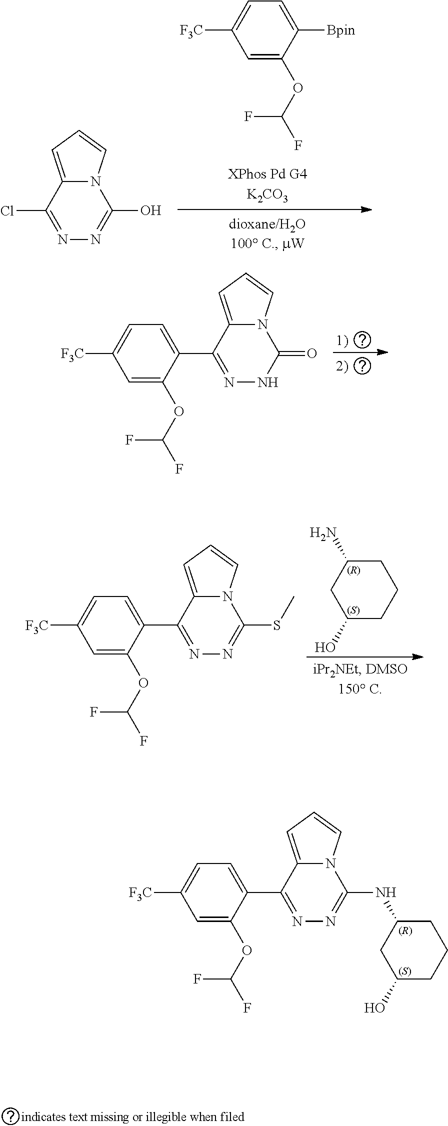

### Step 1: 1-[2-(Difluoromethoxy)-4-(trifluoromethyl)phenyl]-3H-pyrrolo[1,2-d][1,2,4]triazin-4-one

To a solution of 1-chloropyrrolo[1,2-d][1,2,4]triazin-4-ol (Intermediate 2d, step 1, 667 mg, 3.93 mmol, 1.0 eq.) in 1,4-dioxane (12 mL, 0.3 M) and water (3 mL) was added 2-(2-(difluoromethoxy)-4-(trifluoromethyl) phenyl)-4,4,5,5-tetramethyl-1,3,2-dioxaborolane (Intermediate 7a, 2.00 g, 5.89 mmol, 1.5 eq.), K₂CO₃ (1.10 g, 7.86 mmol, 2.0 eq.) and XPhosPdG4 (333 mg, 0.390 mmol, 0.1 eq.). After the addition was completed, the reaction mixture was stirred at 100° C. for 15 minutes under microwave under N₂. The mixture was diluted with water (20 mL) and extracted with EA (20 mL×3). The organic layer was washed with brine, dried over anhydrous Na₂SO₄, and evaporated in vacuo. The residue was purified by flash chromatography on silica gel (PE:EA=10:1 to 5:1) to obtain 1-(2-(difluoromethoxy)-4-(trifluoromethyl) pheny) pyrrolo[1,2-d][1,2,4] triazin-4(3H)-one (1.20 g, 3.47 mmol, 88% yield) as a yellow solid. ¹H NMR (400 MHz, DMSO-d6) δ: 12.67 (s, 1H), 7.86-7.84 (m, 2H), 7.79 (d, J=8.0 Hz, 1H), 7.74 (s, 1H), 7.38 (t, J=73.2 Hz, 1H), 6.86-6.84 (m, 1H), 6.57 (dd, J=1.2 Hz, 1H).

### Step 2. 1-[2-(Difluoromethoxy)-4-(trifluoromethyl)phenyl]-3H-pyrrolo[1,2-d][1,2,4]triazine-4-thione

To 1-[2-(Difluoromethoxy)-4-(trifluoromethyl)phenyl]-3H-pyrrolo[1,2-d][1,2,4]triazin-4-one (1.34 g, 3.88 mmol) in PhMe (16 mL) was added Lawesson's reagent (1.05 g, 2.52 mmol). The mixture was heated to 120° C. for 10 h. The reaction was cooled to rt and filtered to give a white solid (1.15 g, 82%). MS m/z 362.3 [M+H]⁺.

### Step 3: 1-[2-(Difluoromethoxy)-4-(trifluoromethyl)phenyl]-4-methylsulfanyl-pyrrolo[1,2-d][1,2,4]triazine

To a solution of 1-[2-(difluoromethoxy)-4-(trifluoromethyl)phenyl]-3H-pyrrolo[1,2-d][1,2,4]triazine-4-thione (0.500 g, 1.38 mmol) in THF/H₂O (2:1, 3 mL) was added MeI (0.151 mL, 2.42 mmol) and K₂CO₃ (0.287 g, 2.08 mmol). The mixture was stirred at rt for 2 h, then diluted with EtOAc, washed with brine, dried (Na₂SO₄), filtered, and concentrated. Purification by chromatography on SiO₂ (EtOAc:hexanes, 20-60%) gave a white solid (0.419 g, 81%). MS m/z 376.3 [M+H]⁺; ¹H NMR (400 MHz, DMSO-d₆) δ 7.94-7.74 (m, 4H), 7.39 (t, J=73.17 Hz, 1H), 7.13 (t, J=3.30 Hz, 1H), 6.75 (d, J=3.60 Hz, 1H), 2.87 (s, 3H).

### Step 4: (1S,3R)-3-[[1-[2-(Difluoromethoxy)-4-(trifluoromethyl)phenyl]pyrrolo[1,2-d][1,2,4]triazin-4-yl]amino]cyclohexanol

A solution of (difluoromethoxy)-4-(trifluoromethyl)phenyl]-4-methylsulfanyl-pyrrolo[1,2-d][1,2,4]triazine (0.070 g, 0.19 mmol) and (1S,3R)-3-aminocyclohexanol hydrochloride (0.071 g, 0.47 mmol) in DMSO (0.31 mL) was added iPr₂NEt (0.13 mL, 0.75 mmol). The reaction was heated to 150° C. for 36 h, then diluted with DCM/iPrOH(9:1) and washed with brine, dried (Na₂SO₄), filtered, and concentrated. Purification by chromatography on SiO₂ (MeOH:DCM, 0 to 10%) followed by reverse phase chromatography (0.1% formic acid in MeCN:0.1% formic acid in H₂O, 5 to 100%) gave a pale-yellow solid (0.041 g, 50%). MS m/z 443.4 [M+H]⁺; ¹H NMR (400 MHz, DMSO-d₆) δ 7.95 (s, 1H), 7.85-7.73 (m, 2H), 7.73-7.66 (s, 1H), 7.42 (d, J=7.9 Hz, 1H), 7.36 (t, J=73.8 Hz, 1H), 6.95 (s, 1H), 6.47 (d, J=3.5 Hz, 1H), 4.73 (d, J=3.9 Hz, 1H), 4.23-4.12 (m, 1H), 3.60-3.49 (m, 1H), 2.35-2.24 (m, 1H), 2.10-1.96 (m, 1H), 1.93-1.82 (m, 1H), 1.81-1.70 (m, 1H), 1.44-1.05 (m, 4H).

### Example 33

### Preparation of Compound I-322

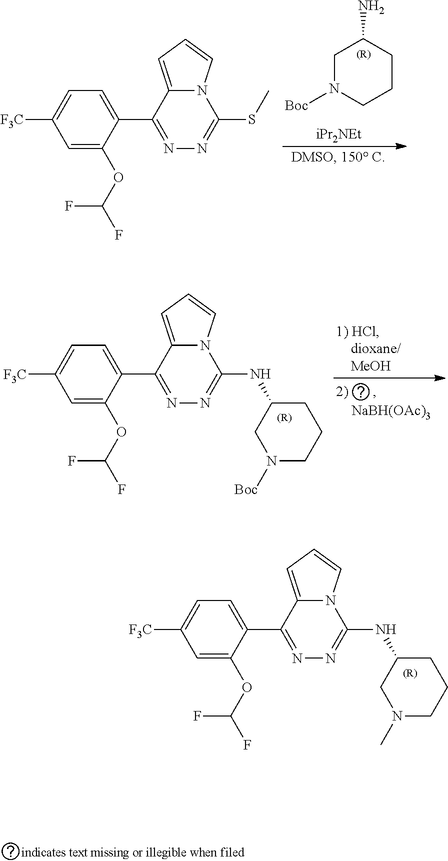

### Step 1: t-Butyl (3R)-3-[[1-[2-(difluoromethoxy)-4-(trifluoromethyl)phenyl]pyrrolo[1,2-d][1,2,4]triazin-4-yl]amino]piperidine-1-carboxylate

A solution of (difluoromethoxy)-4-(trifluoromethyl)phenyl]-4-methylsulfanyl-pyrrolo[1,2-d][1,2,4]triazine (Example 32, step 1-2, 0.20 g, 0.53 mmol) and t-butyl (3R)-3-aminopiperidine-1-carboxylate (0.213 g, 1.07 mmol) in DMSO (1.0 mL) was added iPr₂NEt (0.23 mL, 1.60 mmol). The reaction was heated to 150° C. for 36 h. The reaction was diluted with DCM/iPrOH(9:1) and washed with brine, water, brine, dried (Na₂SO₄), filtered and concentrated. MS m/z 528.3 [M+H]⁺.

### Step 2. 1-[2-(Difluoromethoxy)-4-(trifluoromethyl)phenyl]-N-[(3R)-1-methyl-3-piperidyl]pyrrolo[1,2-d][1,2,4]triazin-4-amine

t-Butyl (3R)-3-[[1-[2-(difluoromethoxy)-4-(trifluoromethyl)phenyl]pyrrolo[1,2-d][1,2,4]triazin-4-yl]amino]piperidine-1-carboxylate (0.281 g, 0.533 mmol) was dissolved in dioxane (0.5 mL) and added HCl (4M in dioxane, 1.5 mL). The reaction was stirred at rt for 1.5 h, then diluted with DCM/iPrOH(9:1) and washed with NaHCO₃, H₂O, and brine. The organics were dried (Na₂SO₄), filtered, and concentrated to give a brown oil. The oil was then dissolved in DCM (5 mL) was added a methanolic formaldehyde solution (formaldehyde 37% in water, 0.12 mL, in MeOH 0.12 mL) followed by NaBH(OAc)₃ (0.424 g, 1.60 mmol). The reaction was stirred for 10 min before dilution with DCM. The mixture was then washed with sat. NaHCO₃, water, and brine. The organic phase was dried (Na₂SO₄), filtered, and concentrated. Purification by reverse phase chromatography (0.1% formic acid in MeCN:0.1% formic acid in H₂O, 5 to 100%) gave an off-white solid (0.063 g, 27%). MS m/z 442.4 [M+H]⁺; ¹H NMR (400 MHz, DMSO-d₆) δ 7.99 (s, 1H), 7.79 (q, J=7.1 Hz, 2H), 7.70 (s, 1H), 7.37 (t, J=73.92 Hz, 1H), 7.33 (d, J=7.5 Hz, 1H), 6.95 (s, 1H), 6.48 (d, J=3.6 Hz, 1H), 4.38-4.25 (m, 1H), 3.08 (d, J=8.9 Hz, 1H), 2.72 (d, J=11.0 Hz, 1H), 2.22 (s, 3H), 2.07-1.98 (m, 1H), 1.98-1.86 (m, 2H), 1.81-1.70 (m, 1H), 1.67-1.53 (m, 1H), 1.50-1.35 (m, 1H).

### Example 34

### Preparation of Compound I-325

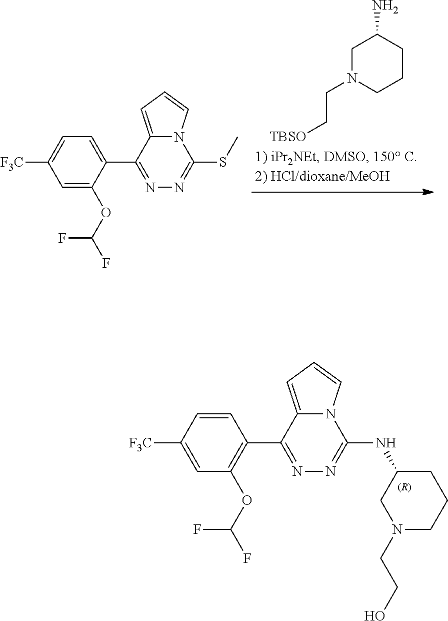

### Step 1: 2-[(3R)-3-[[1-[2-(Difluoromethoxy)-4-(trifluoromethyl)phenyl]pyrrolo[1,2-d][1,2,4]triazin-4-yl]amino]-1-piperidyl]ethanol formic acid salt

A mixture of (difluoromethoxy)-4-(trifluoromethyl)phenyl]-4-methylsulfanyl-pyrrolo[1,2-d][1,2,4]triazine (Example 32, step 1-2, 0.060 g, 0.16 mmol) and (3R)-1-[2-[t-butyl(dimethyl)silyl]oxyethyl]piperidin-3-amine (0.083 g, 0.32 mmol) in DMSO (0.30 mL) and iPr₂NEt (0.11 mL, 0.64 mmol) was heated to 150° C. for 18 h. The reaction was cooled to rt and diluted with DCM/iPrOH (9:1). The solution was washed with water, brine, dried (Na₂SO₄), filtered and concentrated. The resulting oil was dissolved in MeOH (0.5 mL) and added HCl/dioxane (4M, 1.0 mL). The reaction was stirred for 1 h. The mixture was concentrated, and the resulting residue was dissolved in DCM/iPrOH (9:1). The solution was washed with sat. NaHCO₃ and brine, dried (Na₂SO₄), filtered, and concentrated. Purification by chromatography on SiO₂ (MeOH:DCM, 0 to 10%) followed by reverse phase chromatography (0.1% formic acid in MeCN:0.1% formic acid in H₂O, 5 to 100%) gave an off-white solid (0.031 g, 37%). MS m/z 472.2 [M+H]⁺; ¹H NMR (400 MHz, DMSO-d₆) δ 8.18 (s, 1H, formic acid), 7.97 (s, 1H), 7.80 (q, J=8.0 Hz, 2H), 7.70 (s, 1H), 7.37 (t, J=73.54 Hz, 1H), 7.31 (d, J=7.50 HZ, 1H), 6.95 (s, 1H), 6.48 (d, J=3.5 Hz, 1H), 4.55-4.23 (m, 2H), 3.52 (t, J=6.1 Hz, 2H), 3.17 (d, J=9.8 Hz, 1H), 2.83 (d, J=10.5 Hz, 1H), 2.47 (t, J=6.6 Hz, 2H), 2.13-1.98 (m, 3H), 1.82-1.70 (m, 1H), 1.68-1.39 (m, 2H).

### Example 35

### Preparation of Compounds I-323 and I-324

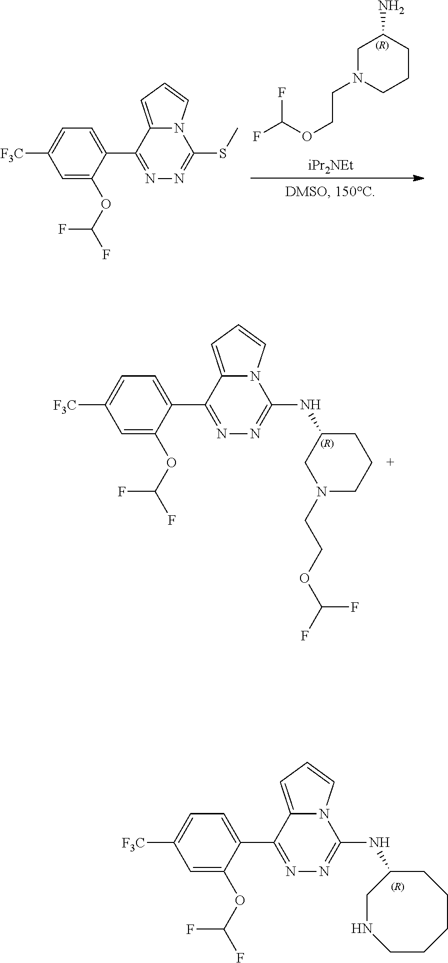

A mixture of (difluoromethoxy)-4-(trifluoromethyl)phenyl]-4-methylsulfanyl-pyrrolo[1,2-d][1,2,4]triazine (Example 32, step 1-2, 0.060 g, 0.16 mmol) and (3R)-1-[2-(difluoromethoxy)ethyl]piperidin-3-amine hydrochloride (0.074 g, 0.32 mmol) in DMSO (0.30 mL) and iPr₂NEt (0.11 mL, 0.64 mmol) was heated to 150° C. for 18 h. The reaction was cooled to rt and diluted with DCM/iPrOH (9:1). The solution was washed with brine, dried (Na₂SO₄), filtered, and concentrated. Purification by reverse phase chromatography (0.1% formic acid in MeCN:0.1% formic acid in H₂O, 5 to 100%) provided 1-[2-(difluoromethoxy)-4-(trifluoromethyl)phenyl]-N-[(3R)-1-[2-(difluoromethoxy)ethyl]-3-piperidyl]pyrrolo[1,2-d][1,2,4]triazin-4-amine formic acid salt (0.004 g, 4%) and 1-[2-(difluoromethoxy)-4-(trifluoromethyl)phenyl]-N-[(3R)-azocan-3-yl]pyrrolo[1,2-d][1,2,4]triazin-4-amine formic acid salt, respectively.

Compound I-323: MS m/z 522.3 [M+H]⁺; ¹H NMR (400 MHz, DMSO-d₆) δ 8.32 (s, 1H, formic acid), 7.97 (s, 1H), 7.85-7.74 (m, 2H), 7.70 (s, 1H), 7.37 (t, J=73.79 Hz, 1H), 7.34 (d, J=8.13 Hz, 1H), 6.99-6.92 (m, 1H), 6.67 (t, J=77.04 Hz, 1H), 6.50-6.48 (m, 1H), 4.38-4.25 (m, 1H), 3.94 (t, J=5.6 Hz, 2H), 3.19 (d, J=10.6 Hz, 1H), 2.86 (d, J=10.8 Hz, 1H), 2.69-2.60 (m, 2H), 2.17-1.96 (m, 3H), 1.87-1.71 (m, 1H), 1.68-1.37 (m, 2H).

Compound I-324: MS m/z 456.2 [M+H]⁺; ¹H NMR (400 MHz, DMSO-d6) δ 8.05 (d, J=19.76 Hz, 1H), 7.99 (d, J=11.76 Hz, 1H), 7.86-7.74 (m, 2H), 7.74-7.68 (m, 1H), 7.48 (t, J=7.00 Hz, 1H), 7.37 (t, J=73.04 Hz, 1H), 7.03-6.94 (m, 1H), 6.55-6.47 (m, 1H), 4.50-4.40 (m, 1H), 4.29-4.10 (m, 1H), 3.99 (d, J=12.3 Hz, 1H), 3.71-3.61 (m, 1H), 3.21-3.03 (m, 2H), 2.92-2.72 (m, 2H), 2.17 (ddd, J=10.4, 4.2, 2.3 Hz, 1H), 1.93-1.69 (m, 3H), 1.62-1.40 (m, 1H)

### Example 36

### Preparation of Compound I-356

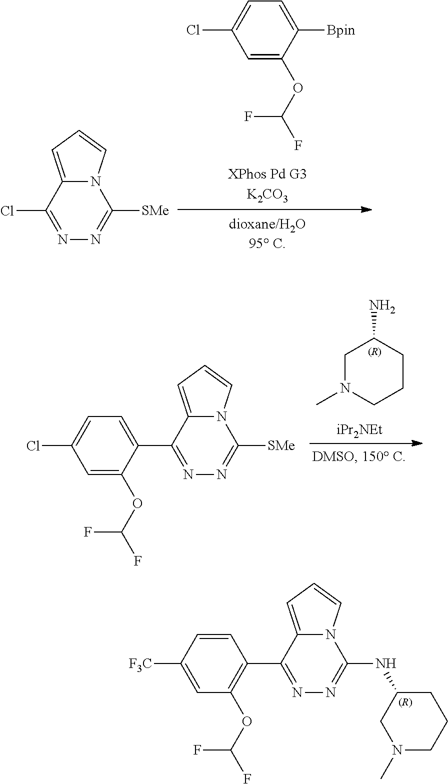

### Step 1: 1-[4-Chloro-2-(difluoromethoxy)phenyl]-4-methylsulfanyl-pyrrolo[1,2-d][1,2,4]triazine

1-Chloro-4-methylsulfanyl-pyrrolo[1,2-d][1,2,4]triazine (Intermediate 2d, 0.200 g, 1.00 mmol), 2-[4-chloro-2-(difluoromethoxy)phenyl]-4,4,5,5-tetramethyl-1,3,2-dioxaborolane (prepared according to the procedure of Intermediate 7a, 0.46 g, 1.50 mmol), and XPhos Pd G3 (0.087 g, 0.100 mmol) were placed in a vial and evacuated and refilled with Ar. Dioxane (5 mL) and aq. K₂CO₃ (2M, 1.50 mL, 3.01 mmol) were added and the solution was sparged with Ar for 5 min. The reaction was heated to 95° C. for 4 h. The reaction was cooled to rt and diluted with EtOAc. The solution was washed with water and brine. The organic phase was dried (Mg₂SO₄), filtered, and concentrated. Purification by chromatography on SiO₂ (EtOAc:hexanes, 0 to 60%) gave a tan foam (0.202 g, 59%). MS m/z 342.2, 344.2 [M+H]⁺.

### Step 2. 1-[4-Chloro-2-(difluoromethoxy)phenyl]-N-[(3R)-1-methyl-3-piperidyl]pyrrolo[1,2-d][1,2,4]triazin-4-amine

A mixture of 1-[4-chloro-2-(difluoromethoxy)phenyl]-4-methylsulfanyl-pyrrolo[1,2-d][1,2,4]triazine (0.042 g, 0.12 mmol) and (3R)-1-methylpiperidin-3-amine (0.042 g, 0.37 mmol) in DMSO (0.25 mL) and iPr₂NEt (86 mL, 0.49 mmol) was heated to 150° C. for 30 h. The reaction was cooled to rt and diluted with DCM/iPrOH (9:1). The solution was washed with brine, dried (Na₂SO₄), filtered, and concentrated. Purification by chromatography on SiO₂ (MeOH:DCM, 0 to 10%) followed by reverse phase chromatography (0.1% formic acid in MeCN:0.1% formic acid in H₂O, 5 to 100%) gave a white solid (0.012 g, 24%). MS m/z 408.4 [M+H]⁺; ¹H NMR (400 MHz, DMSO-d₆) δ 7.96 (s, 1H), 7.64-7.55 (m, 1H), 7.48 (s, 2H), 7.29 (s, 1H), 7.26 (t, J=63.5 Hz, 1H), 6.93 (s, 1H), 6.44 (d, J=2.9 Hz, 1H), 4.37-4.23 (m, 1H), 3.07 (d, J=8.1 Hz, 1H), 2.72 (d, J=10.3 Hz, 1H), 2.22 (s, 3H), 2.06-1.85 (m, 3H), 1.85-1.70 (m, 1H), 1.69-1.51 (m, 1H), 1.50-1.35 (m, 1H).

### Example 37

### Preparation of Compounds I-443, I-425, and I-446

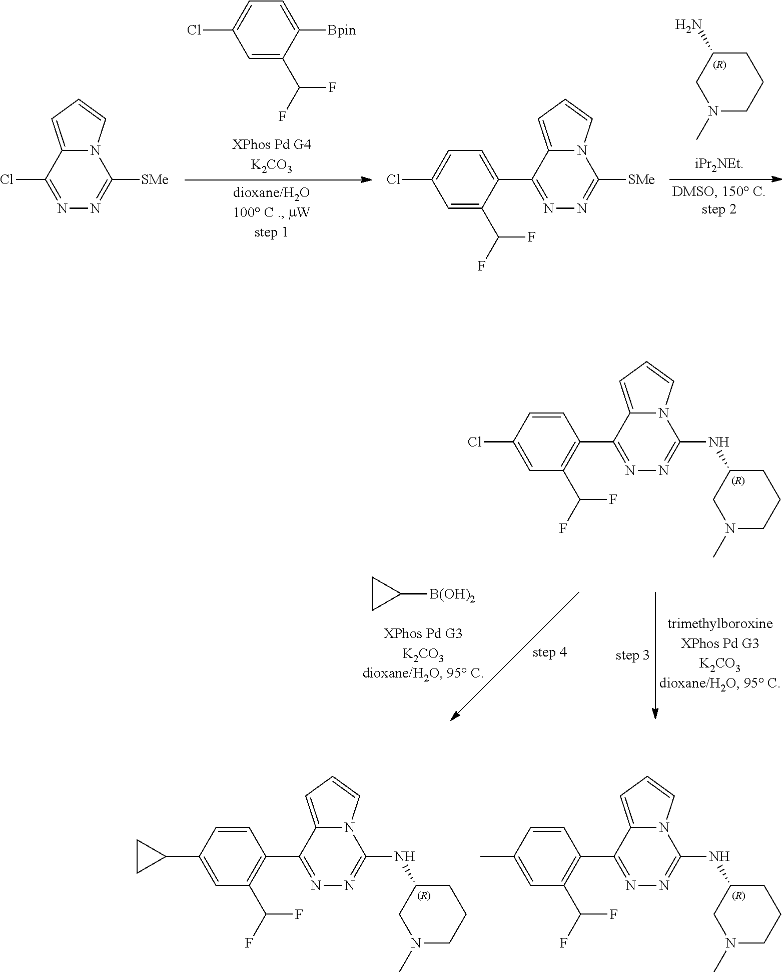

### Step 1: 1-[4-Chloro-2-(difluoromethyl)phenyl]-4-methylsulfanyl-pyrrolo[1,2-d][1,2,4]triazine

The title compound was prepared in analogous manner according to the procedure of Example 36, step 1, using 2-(4-chloro-2-(difluoromethyl)phenyl)-4,4,5,5-tetramethyl-1,3,2-dioxaborolane (Intermediate 6a) in replace of 2-[4-chloro-2-(difluoromethoxy)phenyl]-4,4,5,5-tetramethyl-1,3,2-dioxaborolane. MS m/z 326.0 [M+H]⁺; ¹H NMR (400 MHz, DMSO-d6) δ 7.89 (s, 1H), 7.81 (d, J=4.0 Hz, 3H), 7.14 (t, J=54.8 Hz, 1H), 7.14 (d, J=6.8 Hz, 1H), 6.78 (d, J=3.6 Hz, 1H), 2.86 (s, 3H).

### Step 2. 1-[4-Chloro-2-(difluoromethyl)phenyl]-N-[(3R)-1-methyl-3-piperidyl]pyrrolo[1,2-d][1,2,4]triazin-4-amine

A mixture of 1-[4-chloro-2-(difluoromethyl)phenyl]-4-methylsulfanyl-pyrrolo[1,2-d][1,2,4]triazine (0.415 g, 1.27 mmol) and (3R)-1-methylpiperidin-3-amine (0.364 g, 3.18 mmol) in DMSO (1.3 mL) and iPr₂NEt (0.67 mL, 3.82 mmol) was heated to 150° C. for 24 h. The reaction was cooled to rt and diluted with DCM/iPrOH (9:1). The solution was washed with brine, dried (Na₂SO₄), filtered, and concentrated. Purification by chromatography on SiO₂ (MeOH:DCM, 0 to 10%) followed by reverse phase chromatography (0.1% formic acid in MeCN:0.1% formic acid in H₂O, 5 to 100%) gave a pale-yellow solid (0.239 g, 48%). MS m/z 392.1, 394.1 [M+H]⁺; ¹H NMR (400 MHz, DMSO-d₆) δ 8.01 (s, 1H), 7.83 (s, 1H), 7.79-7.72 (m, 2H), 7.47-7.31 (m, 1H), 7.14 (t, J=55.3 Hz, 1H), 6.97 (s, 1H), 6.56 (s, 1H), 4.40-4.27 (m, 1H), 3.10 (d, J=9.1 Hz, 1H), 2.75 (d, J=9.4 Hz, 1H), 2.25 (s, 3H), 2.10-1.91 (m, 3H), 1.82-1.71 (m, 1H), 1.67-1.53 (m, 1H), 1.51-1.34 (m, 1H).

### Step 3: 1-[2-(Difluoromethyl)-4-methyl-phenyl]-N-[(3R)-1-methyl-3-piperidyl]pyrrolo[1,2-d][1,2,4]triazin-4-amine

A mixture of 1-[4-chloro-2-(difluoromethyl)phenyl]-N-[(3R)-1-methyl-3-piperidyl]pyrrolo[1,2-d][1,2,4]triazin-4-amine (0.040 g, 0.100 mmol), 2,4,6-trimethyl-1,3,5,2,4,6-trioxatriborinane (0.071 mL, 0.51 mmol), and XPhos Pd G3 (0.009 g, 0.010 mmol) was evacuated and backfilled with Ar (3×) before being diluted with dioxane (0.5 mL) and aq. K₂CO₃ (2M, 0.15 mL, 0.31 mmol). The mixture was sparged with Ar for 5 min. The reaction was then heated to 95° C. for 17 h. The mixture was diluted with EtOAc and filtered through Celite. The filtrate was washed with brine and concentrated. Purification by chromatography on SiO₂ (10% NH₄OH in MeOH:DCM, 0 to 10%) followed by reverse phase chromatography (0.1% formic acid in MeCN:0.1% formic acid in H₂O, 5 to 100%) gave a white solid (0.026 g, 69%). MS m/z 372.3 [M+H]⁺; ¹H NMR (400 MHz, CD₃OD) δ 7.86 (s, 1H), 7.66 (s, 1H), 7.54-7.46 (m, 2H), 6.99 (s, 1H), 6.87 (t, J=58.78 Hz, 1H), 6.58 (s, 1H), 4.58-4.45 (m, 1H), 3.67-3.51 (m, 1H), 3.25-3.11 (m, 1H), 2.91-2.75 (m, 2H), 2.72 (s, 3H), 2.50 (s, 3H), 2.26-2.13 (m, 1H), 2.13-2.02 (m, 1H), 1.96-1.72 (m, 2H). 1H not observed (NH).

### Step 4: 1-[4-Cyclopropyl-2-(difluoromethyl)phenyl]-N-[(3R)-1-methyl-3-piperidyl]pyrrolo-[1,2-d][1,2,4]triazin-4-amine

A mixture of 1-[4-chloro-2-(difluoromethyl)phenyl]-N-[(3R)-1-methyl-3-piperidyl]pyrrolo[1,2-d][1,2,4]triazin-4-amine (0.040 g, 0.100 mmol), cyclopropylboronic acid (0.071 mL, 0.51 mmol), and XPhos Pd G3 (0.009 g, 0.010 mmol) was evacuated and backfilled with Ar (3×) before being diluted with dioxane (0.5 mL) and aq. K₂CO₃ (2M, 0.15 mL, 0.31 mmol). The mixture was sparged with Ar for 5 min. The reaction was then heated to 95° C. for 17 h. The mixture was diluted with EtOAc and filtered through Celite. The filtrate was washed with brine and concentrated. Purification by chromatography on SiO₂ (10% NH₄OH in MeOH:DCM, 0 to 10%) followed by reverse phase chromatography (0.1% formic acid in MeCN:0.1% formic acid in H₂O, 5 to 100%) gave a white solid (0.093 g, 41%). MS m/z 398.2 [M+H]⁺; ¹H NMR (400 MHz, DMSO-d₆) δ 7.98 (s, 1H), 7.59-7.54 (m, 1H), 7.54-7.50 (m, 1H), 7.37-7.30 (m, 1H), 7.30-7.25 (m, 1H), 7.09 (t, J=55.16 Hz, 1H), 6.95 (s, 1H), 6.51 (s, 1H), 4.38-4.25 (m, 1H), 3.10 (d, J=8.5 Hz, 1H), 2.75 (d, J=10.4 Hz, 1H), 2.25 (s, 3H), 2.16-2.06 (m, 1H), 2.05-1.90 (m, 3H), 1.90-1.71 (m, 1H), 1.71-1.54 (m, 1H), 1.54-1.35 (m, 1H), 1.09-1.01 (m, 2H), 0.84-0.75 (m, 2H).

### Example 38

### Preparation of Compound I-342

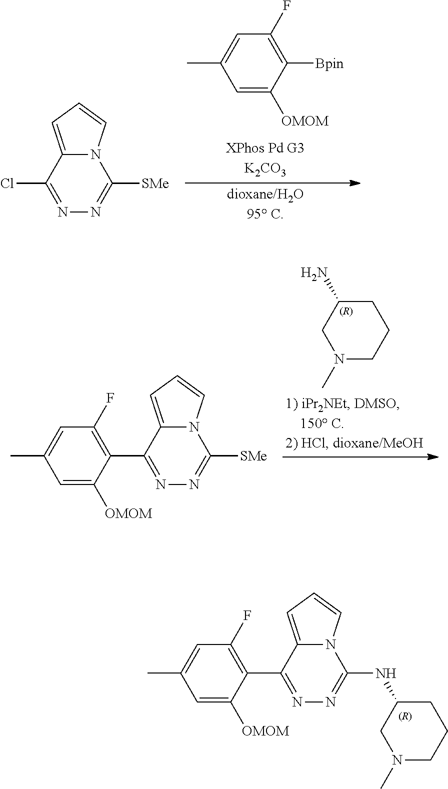

### Step 1: 1-[2-Fluoro-6-(methoxymethoxy)-4-methyl-phenyl]-4-methylsulfanyl-pyrrolo[1,2-d][1,2,4]triazine

1-Chloro-4-methylsulfanyl-pyrrolo[1,2-d][1,2,4]triazine (Intermediate 2d, 0.500 g, 2.50 mmol), 2-[2-fluoro-6-(methoxymethoxy)-4-methyl-phenyl]-4,4,5,5-tetramethyl-1,3,2-dioxaborolane (prepared according to the procedure for Intermediate 5d, 1.11 g, 3.76 mmol), and XPhos Pd G3 (0.216 g, 0.250 mmol) were placed in a vial and evacuated and refilled with Ar. Dioxane (12.5 mL) and aq. K₂CO₃ (2M, 0.3.8 mL, 7.51 mmol) were added and the solution was sparged with Ar for 5 min. The reaction was heated to 95° C. for 15 h, then cooled to rt and diluted with EtOAc. The solution was washed with water and brine. The organic phase was dried (Mg₂SO₄), filtered, and concentrated. Purification by chromatography on SiO₂ (EtOAc:hexanes, 0 to 60%) gave a tan solid (0.481 g, 58%). MS m/z 334.3 [M+H]⁺.

### Step 2. 3-Fluoro-5-methyl-2-[4-[[(3R)-1-methyl-3-piperidyl]amino]pyrrolo[1,2-d][1,2,4]triazin-1-yl]phenol

A mixture of 1-[2-fluoro-6-(methoxymethoxy)-4-methyl-phenyl]-4-methylsulfanyl-pyrrolo[1,2-d][1,2,4]triazine (0.095 g, 0.28 mmol) and (3R)-1-methylpiperidin-3-amine (0.098 g, 0.85 mmol) in DMSO (0.47 mL) and iPr₂NEt (0.20 mL, 1.1 mmol) was heated to 150° C. for 23 h. The reaction was cooled to rt and diluted with DCM/iPrOH (9:1). The solution was washed with water, brine, dried (Na₂SO₄), filtered and concentrated. The resulting oil was dissolved in MeOH (0.5 mL) and added HCl/dioxane (4M, 1.0 mL). The reaction was stirred for 1 h. The mixture was concentrated, and the resulting residue was dissolved in DCM/iPrOH (9:1). The solution was washed with sat. NaHCO₃ and brine, dried (Na₂SO₄), filtered and concentrated. Purification by chromatography on SiO₂ (MeOH:DCM, 0 to 10%) followed by reverse phase chromatography (0.1% formic acid in MeCN:0.1% formic acid in H₂O, 5 to 100%) gave an off-white solid (0.020 g, 20%). MS m/z 356.2 [M+H]⁺; ¹H NMR (400 MHz, DMSO-d₆) δ 9.94 (br s, 1H), 7.91 (s, 1H), 7.17 (d, J=7.9 Hz, 1H), 6.88 (s, 1H), 6.63-6.55 (m, 2H), 6.30 (d, J=4.00 Hz, 1H), 4.36-4.21 (m, 1H), 3.13-3.02 (m, 1H), 2.76-2.62 (m, 1H), 2.30 (s, 3H), 2.21 (s, 3H), 2.06-1.97 (m, 1H), 1.96-1.84 (m, 2H), 1.81-1.71 (m, 1H), 1.66-1.52 (m, 1H), 1.50-1.35 (m, 1H).

### Example 39a

### Preparation of Compound I-501

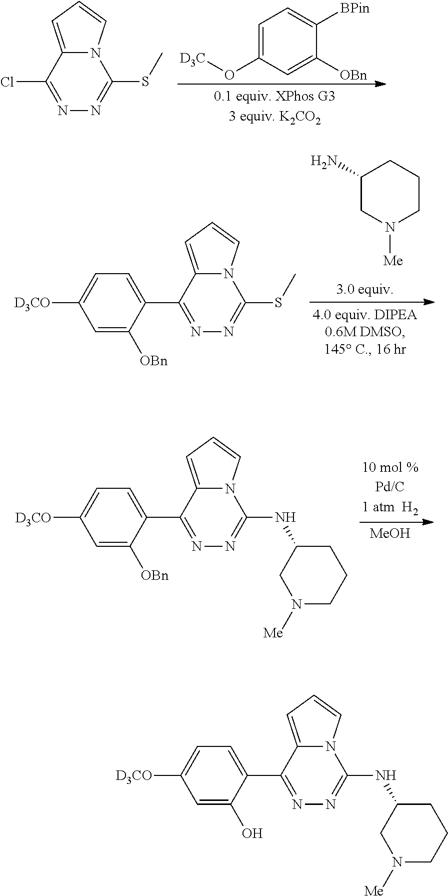

### Step 1: 1-(2-(Benzyloxy)-4-(methoxy-d3)phenyl)-4-(methylthio)pyrrolo[1,2-d][1,2,4]triazine

1-Chloro-4-methylsulfanyl-pyrrolo[1,2-d][1,2,4]triazine (Intermediate 2d, 0.67 g, 3.34 mmol, 1.5 eq.), 2-(2-(benzyloxy)-4-(methoxy-d3)phenyl)-4,4,5,5-tetramethyl-1,3,2-dioxaborolane (Intermediate 5e, 0.76 g, 2.23 mmol), and XPhos Pd G3 (0.19 g, 0.22 mmol, 0.1 eq.) were placed in a vial and evacuated and refilled with argon. Dioxane (11 mL) and aq. K₂CO₃ (2M, 3.34 mL, 6.67 mmol, 3 eq.) were added and the solution was sparged with argon for 15 min. The reaction was heated to 95° C. for 6 h, then cooled to rt and diluted with EtOAc. The reaction mixture was washed with water and brine. The organic phase was dried (Mg₂SO₄), filtered, and concentrated. Purification by chromatography on SiO₂ (EtOAc:hexanes, 0 to 60%) gave an brown oil (0.59 g, 69%). MS m/z 381.5 [M+H]⁺; ¹H NMR (400 MHz, CHLOROFORM-d) δ 7.66-7.58 (m, 1H), 7.56-7.51 (m, 1H), 7.27 (s, 2H), 7.09-6.93 (m, 3H), 7.09-6.92 (m, 2H), 6.72-6.66 (m, 2H), 5.15 (s, 2H), 2.92 (s, 3H).

### Step 2. (R)-1-(2-(Benzyloxy)-4-(methoxy-d3)phenyl)-N-(1-methylpiperidin-3-yl)pyrrolo[1,2-d][1,2,4]triazin-4-amine

A mixture of 1-(2-(benzyloxy)-4-(methoxy-d3)phenyl)-4-(methylthio)pyrrolo[1,2-d][1,2,4]triazine (0.16 g, 0.42 mmol) and (3R)-1-methylpiperidin-3-amine (0.15 g, 1.27 mmol, 3 eq.) in DMSO (0.71 mL) and iPr₂NEt (0.3 mL, 1.70 mmol, 4 eq.) was heated to 150° C. for 11 h. The reaction was cooled to rt and diluted with DCM/iPrOH (9:1). The solution was washed with brine, dried (Na₂SO₄), filtered, and concentrated. Purification by chromatography on SiO₂ (MeOH: DCM, 0 to 10%) gave a brown oil (0.18 g, 94%). MS m/z 447.7 [M+H]⁺.

### Step 3: (R)-5-(Methoxy-d3)-2-(4-((1-methylpiperidin-3-yl)amino)pyrrolo[1,2-d][1,2,4]triazin-1-yl)phenol

10% Pd/C (0.049 g, 0.047 mmol, 0.1 eq.) was added to a solution of (R)-1-(2-(benzyloxy)-4-(methoxy-d3)phenyl)-N-(1-methylpiperidin-3-yl)pyrrolo[1,2-d][1,2,4]triazin-4-amine (0.21 g, 0.47 mmol) in MeOH (4.7 mL) under nitrogen. Then the nitrogen in reaction vial was replaced with H₂ (balloon). The reaction mixture was allowed to stir at room temperature for 2 h. The reaction mixture was passed through a Celite filter to remove Pd/C. The filtrate was concentrated and purified by reverse phase chromatography (0.1% formic acid in MeCN:0.1% formic acid in H₂O, 5 to 100%) gave yellow solid (0.063 g, 27%). MS m/z 356.1 [M+H]⁺; ¹H NMR (400 MHz, CD₃OD) δ 8.51 (s, 1H, Formic acid peak), 7.92 (d, J=7.9 Hz, 1H), 7.87 (s, 1H), 7.25 (s, 1H), 7.05 (s, 1H), 6.59 (d, J=7.6 Hz, 1H), 6.53 (s, 1H), 4.53-4.36 (m, 1H), 3.52-3.35 (m, 1H), 3.10-2.98 (m, 1H), 2.61 (s, 3H), 2.74-2.54 (m, 2H), 2.23-2.08 (m, 1H), 2.08-1.94 (m, 1H), 1.94-1.80 (m, 1H), 1.80-1.62 (m, 1H). NH and OH not observed.

### Example 39b

### Preparation of Compound I-514

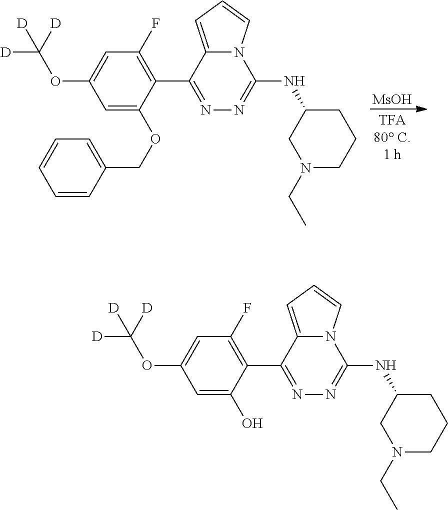 [SMILES: "MsOH" ; [2H]C([2H])([2H])Oc1cc(F)c(-c2nnc(N[C@@H]3CCCN(CC)C3)n3cccc23)c(OCc2ccccc2)c1 ; [2H]C([2H])([2H])Oc1cc(O)c(-c2nnc(N[C@@H]3CCCN(CC)C3)n3cccc23)c(F)c1]

(R)-1-(2-(Benzyloxy)-6-fluoro-4-(methoxy-d₃)phenyl)-N-(1-ethylpiperidin-3-yl)pyrrolo[1,2-d][1,2,4]triazin-4-amine (prepared according to the procedure of Example 46, step 1-3, 14.8 mg, 0.0309 mmol) was dissolved in TFA (1.0 mL) and treated with methanesulfonic acid (29.7 mg, 20.3 μL, 0.309 mmol). The mixture was then stirred at 80° C. for 1 hour. After all starting material has been consumed, the TFA was evaporated, and the acidic residue was diluted and neutralized with saturated NaHCO₃ aqueous solution. The aqueous solution was extracted with CH₂Cl₂ (5×). The combined organic layer was then washed with water and brine, dried over Na₂SO₄, and evaporated under reduced pressure. The crude product was purified by flash column chromatography (gradient elution of CH₂Cl₂:MeOH=100:0 to 80:20) to yield (R)-2-(4-((1-ethylpiperidin-3-yl)amino)pyrrolo[1,2-d][1,2,4]triazin-1-yl)-3-fluoro-5-(methoxy-d₃)phenol (10.8 mg, 90% yield) as pale yellow solid. MS m/z 389.5 [M+H]⁺; ¹H NMR (400 MHz, CD₃OD) δ 7.78 (s, 1H), 6.94 (s, 1H), 6.56 (s, 1H), 6.42-6.30 (m, 2H), 4.54-4.44 (m, 1H), 3.06-2.92 (m, 1H), 2.80-2.64 (m, 2H), 2.57-2.32 (m, 3H), 2.21-2.11 (m, 1H), 2.02-1.92 (m, 1H), 1.87-1.79 (m, 1H), 1.76-1.65 (m, 1H), 1.21 (t, J=7.2 Hz, 3H). 1 OH and 1 NH signals were not observed.

### Example 40

### Preparation of Compound I-498

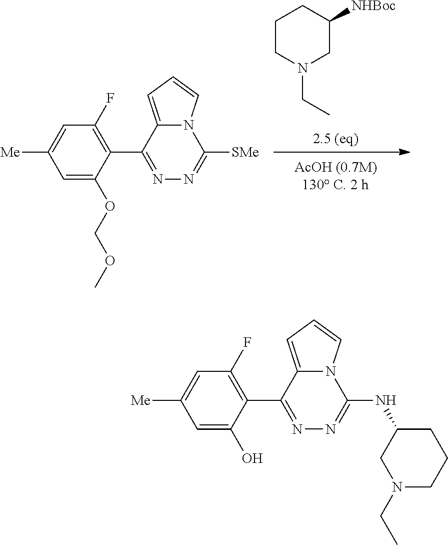 [SMILES: COCOc1cc(C)cc(F)c1-c1nnc(SC)n2cccc12 ; CCN1CCC[C@@H](NC(=O)OC(C)(C)C)C1 ; CCN1CCC[C@@H](Nc2nnc(-c3c(O)cc(C)cc3F)c3cccn23)C1]

To a solution of 1-(2-fluoro-6-(methoxymethoxy)-4-methylphenyl)-4-(methylthio) pyrrolo[1,2-d][1,2,4]triazine (prepared according to the procedure of Example 38, 100 mg, 0.300 mmol, 1.0 eq.) in AcOH (1 mL) was added tert-butyl (R)-(1-ethylpiperidin-3-yl)carbamate (171 mg, 0.750 mmol, 2.5 eq.). The resulting mixture was stirred at 130° C. for 2 h, then filtered and dried under vacuum. The crude was purified by prep-HPLC (MeCN/N₄HCO₃ in water (10 mmol/L)) to give title product (17.0 mg, 0.05 mmol, 16.7% Yield) as a white solid. MS m/z 370.2 [M+H]⁺, ¹H NMR (400 MHz, CD₃OD) δ 7.78 (dd, J=2.8, 1.1 Hz, 1H), 6.95-6.91 (d, 1H), 6.61 (s, 1H), 6.57 (d, J=10.4 Hz, 1H), 6.52 (d, J=3.9 Hz, 1H), 4.52-4.44 (m, 1H), 3.28-3.22 (i, 1H), 2.92-2.78 (m, 1H), 2.63-2.52 (m, 2H), 2.39-2.27 (m, 5H), 2.16-2.07 (m, 1H), 1.942-1.84 (1H), 1.83-1.63 (m, 2H), 1.16 (t, J=7.2 Hz, 3H), NH and OH not observed.

The compounds below were prepared according to the procedure of Example 40 by substituting the appropriate starting materials, reagents and reaction conditions.

| Compound | Spectral Data |
|---|---|
| I-106 | MS m/z 392.1 M + H]⁺; ¹H NMR (CD₃OD) δ: 8.04 (d, J = 8.1 Hz, 1H), 7.81 (d, |
| | J = 2.5 Hz, 1H), 7.10-7.17 (m, 3H), 6.97 (t, J = 3.4 Hz, 1H), 4.28-4.40 (m, 1H), |
| | 3.60-3.67 (m, 1H), 2.94-3.20 (m, 1H), 2.52-2.76 (m, 1H), 2.24-2.29 (m, 3H), |
| | 2.13-2.23 (m, 1H), 1.91-2.08 (m, 1H), 1.59-1.82 (m, 2H), 1.45-1.57 (m, 1H). |
| | NH and OH not observed. |
| I-326 | MS m/z 512.1 [M + H]⁺; ¹H NMR (400 MHz, METHANOL-d4) δ 7.88-7.83 |
| | (m, 1H), 7.83-7.77 (m, 1H), 7.74 (br d, J = 8.1 Hz, 1H), 7.67 (s, 1H), 7.09- |
| | 6.73 (m, 2H), 6.57 (d, J = 3.6 Hz, 1H), 4.55-4.39 (m, 1H), 4.01 (br d, J = 8.3 |
| | Hz, 2H), 3.43 (br t, J = 11.6 Hz, 2H), 2.96-2.84 (m, 1H), 2.72-2.57 (m, 1H), |
| | 2.52-2.29 (m, 2H), 2.15 (br d, J = 11.3 Hz, 1H), 1.94-1.82 (m, 3H), 1.82- |
| | 1.71 (m, 1H), 1.65 (br s, 3H); NH wasn't observedm/z |
| I-331 | MS m/z 364.2 [M + H]⁺; ¹H NMR (METHANOL-d4) δ: 8.44 (s, 1H), 8.36-8.57 |
| | (m, 1H), 7.69-7.81 (m, 2H), 7.11 (br d, J = 3.3 Hz, 1H), 6.94 (br s, 1H), 6.52- |
| | 6.66 (m, 1H), 4.32 (br s, 1H), 2.69-2.89 (m, 1H), 2.45 (br s, 1H), 2.38 (s, 3H), |
| | 1.95-2.07 (m, 1H), 1.86-1.88 (m, 1H), 1.78-1.89 (m, 1H), 1.75-1.92 (m, 1H), |
| | 1.65-1.65 (m, 2H), 1.72 (br s, 1H), 1.49-1.61 (m, 1H), 0.91 (br d, J = 7.9 Hz, |
| | 2H), 0.64 (br d, J = 4.6 Hz, 2H) |
| I-335 | MS m/z 456.4 [M + H]⁺; ¹H NMR (400 MHz, DMSO-d6) δ 7.97 (br s, 1H), 7.80 |
| | (q, J = 7.9 Hz, 2H), 7.70 (s, 1H), 7.37 (t, J = 73.42 Hz, 1 H), 7.36-7.27 (m, 1 |
| | H), 6.96 (br s, 1H), 6.49 (br s, 1H), 4.41-4.21 (m, 1H), 3.59-3.41 (m, 1H), |
| | 2.71-2.55 (m, 1H), 2.43-2.29 (m, 1H), 2.11-1.88 (m, 2H), 1.85-1.65 (m, |
| | 2H), 1.65-1.31 (m, 3H), 1.10-0.90 (m, 3H) |
| I-337 | MS m/z 338.2 [M + H]⁺; ¹H NMR (400 MHz, CD₃OD) δ 7.88 (d, J = 7.6 Hz, |
| | 2H), 7.23 (d, J = 4.0 Hz, 1H), 7.06 (t, J = 3.5 Hz, 1H), 6.84 (d, J = 6.5 Hz, 2H), |
| | 4.50-4.36 (m, 1H), 3.23-3.07 (m, 1H), 2.85-2.71 (m, 1H), 2.50-2.23 (m, |
| | 7H), 2.16-2.04 (m, 1H), 2.00-1.51 (m, 4H). NH and OH not observed. |
| I-340 | MS m/z 434.3 [M + H]⁺; ¹H NMR (400 MHz, METHANOL-d4) δ = 7.92-7.84 |
| | (m, 2H), 7.22 (d, J = 3.6 Hz, 1H), 7.04 (br s, 1H), 6.77-6.65 (m, 2H), 4.39 (td, |
| | J = 4.6, 8.8 Hz, 1H), 4.07-3.93 (m, 2H), 3.42 (br t, J = 11.7 Hz, 2H), 3.27 (br |
| | d, J = 10.5 Hz, 1H), 2.88 (br d, J = 11.1 Hz, 1H), 2.62 (br t, J = 11.6 Hz, 1H), |
| | 2.52-2.32 (m, 2H), 2.13 (br d, J = 8.5 Hz, 1H), 1.95-1.85 (m, 4H), 1.81-1.70 |
| | (m, 1H), 1.63 (br d, J = 12.3 Hz, 3H), 1.02 (br d, J = 8.1 Hz, 2H), 0.76 (br d, J = |
| | 5.1 Hz, 2H); NH and OH not observed |
| I-341 | MS m/z 386.2 [M + H]⁺; ¹H NMR (400 MHz, DMSO-d6) δ 9.95 (br s, 1H), 7.90 |
| | (br s, 1H), 7.15 (d, J = 8.5 Hz, 1H), 6.88 (s, 1H), 6.63-6.56 (m, 2H), 6.31 (d, |
| | J = 4.13 Hz, 1H), 4.40 (t, J = 6.4 Hz, 1H), 4.36-4.22 (m, 1H), 3.52 (q, J = 5.6 |
| | Hz, 2H), 3.20-3.11 (m, 1H), 2.87-2.77 (m, 1H), 2.45 (t, J = 7.75 Hz, 2H), |
| | 2.30 (s, 3H), 2.11-1.96 (m, 3H), 1.80-1.68 (m, 1H), 1.67-1.51 (m, 1H), |
| | 1.51-1.38 (m, 1H). |
| I-343 | MS m/z 462.4 [M + H]⁺; ¹H NMR (400 MHz, METHANOL-d4) δ = 8.04 (br d, |
| | J = 7.9 Hz, 1H), 7.89 (br s, 1H), 7.25-7.13 (m, 3H), 7.05 (br s, 1H), 4.43 (br s, |
| | 1H), 4.01 (br d, J = 8.6 Hz, 2H), 3.42 (br t, J = 11.7 Hz, 2H), 3.32-3.25 (m, |
| | 1H), 2.89 (br d, J = 10.8 Hz, 1H), 2.74-2.51 (m, 1H), 2.40 (br t, J = 9.6 Hz, |
| | 2H), 2.18-2.09 (m, 1H), 1.87-1.74 (m, 4H), 1.69-1.54 (m, 3H). NH and OH |
| | not observed |
| I-344 | MS m/z 436.5 [M + H]⁺; ¹H NMR (400 MHz, DMSO-d6) δ 9.94 (s, 1H), 7.89 (s, |
| | 1H), 7.18 (d, J = 7.3 Hz, 1H), 6.88 (s, 1H), 6.70-6.27 (m, 4H), 4.34-4.21 (m, |
| | 1H), 3.94 (t, J = 5.6 Hz, 2H), 3.19 (d, J = 10.3 Hz, 1H), 2.86 (d, J = 11.4 Hz, |
| | 1H), 2.70-2.62 (m, 2H), 2.30 (s, 3H), 2.15-1.99 (m, 3H), 1.84-1.69 (m, |
| | 1H), 1.67-1.52 (m, 1H), 1.52-1.38 (m, 1H) |
| I-348 | MS m/z 444.2 [M + H]⁺; ¹H NMR (400 MHz, DMSO-d6) d: 8.24 (s, 1H), 8.20 |
| | (d, J = 8.4 Hz, 1H), 8.01 (d, J = 2.0 Hz, 1H), 7.91 (d, J = 8.0 Hz, 1H), 7.34 (d, |
| | J = 7.6 Hz, 1H), 6.95 (t, J = 6.4 Hz, 1H), 6.38 (d, J = 3.2 Hz, 1H), 4.35-4.28 |
| | (m, 1H), 3.08-3.06 (m, 1H), 2.73-2.70 (m, 1H), 2.22 (s, 3H), 2.04-2.00 |
| | (m, 1H), 1.97-1.87 (m, 2H), 1.78-1.75 (m, 1H), 1.66-1.56 (m, 1H), 1.48- |
| | 1.38 (m, 1H). |
| I-352 | MS m/z 378.4 [M + H⁺]⁺; ¹H NMR (400 MHz, Methanol-d4) δ 8.09 (d, J = 8.1 |
| | Hz, 1H), 7.91 (s, 1H), 7.34-7.22 (m, 2H), 7.21 (d, J = 4.0 Hz, 1H), 7.10 (s, |
| | 1H), 4.55-4.41 (m, 1H), 3.78-3.66 (m, 1H), 3.37-3.32 (m, 1H), 3.07-2.91 |
| | (m, 2H), 2.36-2.22 (m, 1H), 2.19-2.04 (m, 1H), 1.97-1.83 (m, 2H). NH and |
| | OH not observed |
| I-353 | MS m/z 422.5 [M + H]⁺; ¹H NMR (400 MHz, Methanol-d4) δ 8.12 (d, J = 8.1 |
| | Hz, 1H), 7.91 (s, 1H), 7.30-7.19 (m, 3H), 7.09 (s, 1H), 4.53-4.42 (m, 1H), |
| | 3.29-3.20 (m, 1H), 3.01-2.84 (m, 1H), 2.64-2.37 (m, 4H), 2.20-2.07 (m, |
| | 1H), 2.02-1.91 (m, 1H), 1.89-1.61 (m, 2H). NH and OH not observed |
| I-354 | MS m/z 368.3 [M + H]⁺; ¹H NMR (400 MHz, CD₃OD) δ 7.99-7.78 (m, 2H), |
| | 7.23 (d, J = 4.0 Hz, 1H), 7.05 (d, J = 4.0 Hz, 1H), 6.84 (d, J = 5.9 Hz, 2H), 4.42 |
| | (dq, J = 9.3, 4.4 Hz, 1H), 3.75 (q, J = 6.1 Hz, 2H), 3.20 (d, J = 10.6 Hz, 1H), |
| | 2.79 (s, 1H), 2.68 (qd, J = 13.4, 6.8 Hz, 2H), 2.57-2.43 (m, 2H), 2.36 (s, 3H), |
| | 2.07 (d, J = 10.6 Hz, 1H), 1.99-1.85 (m, 1H), 1.85-1.59 (m, 2H). NH and |
| | OH peak not observed. |
| I-355 | MS m/z 358.2, 360.2 [M + H]⁺; ¹H NMR (500 MHz, CD₃OD) δ 7.92 (dd, J = |
| | 9.4, 3.1 Hz, 2H), 7.22 (d, J = 3.8 Hz, 1H), 7.10 (t, J = 3.5 Hz, 1H), 7.07-6.96 |
| | (m, 2H), 4.53 (d, J = 11.3 Hz, 1H), 3.89-3.73 (m, 1H), 3.51-3.40 (m, 1H), |
| | 3.12-2.97 (m, 2H), 2.90 (d, J = 2.9 Hz, 3H), 2.34-2.13 (m, 2H), 2.05-1.83 |
| | (m, 2H). NH and OH peak not observed. |
| I-357 | MS [M + H]⁺ 354.4; ¹H NMR (400 MHz, METHANOL-d4) δ 7.91 (d, J = 8.6 |
| | Hz, 1H), 7.86 (s, 1H), 7.28-7.21 (m, 1H), 7.05 (s, 1H), 6.58 (d, J = 8.6 Hz, |
| | 1H), 6.53 (s, 1H), 4.52-4.37 (m, 1H), 3.83 (s, 3H), 3.47 (d, J = 8.1 Hz, 1H), |
| | 3.09 (d, J = 10.3 Hz, 1H), 2.79-2.67 (m, 2H), 2.65 (s, 3H), 2.15 (s, 1H), 2.01 |
| | (s, 1H), 1.87 (d, J = 11.5 Hz, 1H), 1.80-1.67 (m, 1H). NH and OH peak not |
| | observed. |
| I-358 | MS [M + H]⁺ 384.4; ¹H NMR (400 MHz, DMSO-d6) δ 8.12-8.03 (m, 1H), |
| | 8.02-7.89 (m, 1H), 7.38-7.24 (m, 2H), 7.12-7.03 (m, 1H), 6.62-6.47 (m, |
| | 2H), 4.40 (t, J = 5.1 Hz, 1H), 4.31-4.20 (m, 1H), 3.79 (s, 3H), 3.52 (q, J = 5.8 |
| | Hz, 2H), 3.20-3.10 (m, 1H), 2.82 (d, J = 11.0 Hz, 1H), 2.46-2.40 (m, 3H), |
| | 2.12-1.96 (m, 3H), 1.79-1.70 (m, 1H), 1.67-1.53 (m, 1H), 1.51-1.38 (m, |
| | 1H). |
| I-362 | MS m/z 457.4 [M + H]⁺; ¹H NMR (400 MHz, DMSO-d6) δ 7.94 (s, 1H), 7.79 (d, |
| | J = 7.0 Hz, 2H), 7.70 (s, 1H), 7.45 (d, J = 7.4 Hz, 1H), 7.37 (t, J = 74.7 Hz, 1H), |
| | 6.95 (s, 1H), 6.48 (d, J = 3.3 Hz, 1H), 4.28-4.14 (m, 1H), 3.30-3.21 (m, 4H), |
| | 2.46-2.45 (m, 1 H), 2.13-2.01 (m, 2H), 1.87-1.77 (m, 1H), 1.43-0.96 (m, |
| | 4H |
| I-364 | MS m/z 450.3 [M + H]⁺; ¹H NMR (400 MHz, METHANOL-d4) δ 8.20-8.10 |
| | (m, 1H), 7.97-7.88 (m, 1H), 7.28-7.24 (m, 3H), 7.11-7.03 (m, 1H), 4.48- |
| | 4.31 (m, 1H), 3.28-3.18 (m, 1H), 2.91-2.80 (m, 1H), 2.40 (s, 3H), 2.11-1.97 |
| | (m, 1H), 1.91-1.70 (m, 2H), 1.70-1.54 (m, 1H), 1.29 (s, 7H); 3H (2OH and |
| | NH) wasn't observed. |
| I-365 | MS m/z 394.4 [M + H]⁺; ¹H NMR (CD₃OD) δ 8.40 (br s, 1H), 7.77 (br d, J = 8.4 |
| | Hz, 1H), 7.49 (br s, 1H), 7.40 (br s, 1H), 6.99-7.10 (m, 2H), 4.42-4.67 (m, 1H), |
| | 3.79, (br d, J = 12.9 Hz, 1H), 3.39-3.47 (m, 1H), 3.10-3.17 (m, 2H), 2.34 (br s, |
| | 1H), 2.09-2.26 (m, 1H), 1.79-2.07 (m, 2H). OH and NH not observed. |
| I-366 | MS m/z 408.4 [M + H]⁺; ¹H NMR (CD₃OD) δ 8.08 (d, J = 8.5 Hz, 1H), 7.92 (br s, |
| | 1H), 7.26 (br s, 1H), 7.09 (br s, 1H), 6.84-6.95 (m, 2H), 4.39-4.51 (m, 1H), |
| | 3.08-3.30 (m, 1H), 2.69-2.90 (m, 1H), 2.41 (s, 3H), 2.26-2.44 (m, 1H), 2.24- |
| | 2.38 (m, 1H), 2.03-2.22 (m, 1H), 1.85-2.00 (m, 1H), 1.80 (br d, J = 9.9 Hz, 1H), |
| | 1.50-1.72 (m, 1H). OH and NH not observed |
| I-367 | MS m/z 396.4 [M + H]⁺; ¹H NMR (400 MHz, DMSO-d6) δ 8.06 (s, 1H), 7.89 (d, |
| | J = 7.9 Hz, 1H), 7.34 (s, 1H), 7.24 (s, 1H), 7.08 (s, 1H), 6.81 (s, 2H), 4.31 (s, |
| | 1H), 3.20-3.12 (m, 1H), 2.91 (d, J = 11.1 Hz, 2H), 2.32 (s, 3H), 2.26 (s, 2H), |
| | 2.19 (d, J = 11.1 Hz, 1H), 2.01 (d, J = 12.1 Hz, 2H), 1.80-1.69 (m, 1H), 1.68- |
| | 1.55 (m, 1H), 1.52-1.33 (m, 2H), 1.11 (s, 6H). |
| I-368 | MS m/z 408.0, 409.7 [M + H]⁺; ¹H NMR (400 MHz, DMSO-d6) δ 8.08 (s, 1H), |
| | 7.98 (d, J = 8.4 Hz, 1H), 7.49 (d, J = 7.7 Hz, 1H), 7.23 (d, J = 4.0 Hz, 1H), 7.10 |
| | (s, 1H), 7.08-6.93 (m, 2H), 6.37-6.02 (m, 1H), 4.29 (s, 1H), 3.28-3.20 (m, |
| | 1H), 3.18 (s, 1H), 2.91 (d, J = 11.3 Hz, 1H), 2.81 (t, J = 15.6 Hz, 2H), 2.22 (q, |
| | J = 12.1 Hz, 2H), 2.04 (d, J = 12.0 Hz, 1H), 1.76 (d, J = 11.9 Hz, 1H), 1.61 (d, |
| | J = 12.9 Hz, 1H), 1.46 (d, J = 12.4 Hz, 1H). |
| I-369 | MS m/z 388.3, 390.3 [M + H]⁺; ¹H NMR (400 MHz, CD₃OD) δ 7.91-7.76 (m, |
| | 2H), 7.14 (d, J = 4.0 Hz, 1H), 6.97 (t, J = 3.3 Hz, 1H), 6.90 (dd, J = 7.5, 1.9 Hz, |
| | 2H), 4.33 (s, 1H), 3.64 (hept, J = 5.6 Hz, 2H), 3.10 (d, J = 10.9 Hz, 1H), 2.70 |
| | (d, J = 11.1 Hz, 1H), 2.64-2.48 (m, 2H), 2.46-2.30 (m, 2H), 2.02-1.88 (m, |
| | 1H), 1.86-1.75 (m, 1H), 1.75-1.56 (m, 2H). NH and OH peak not observed. |
| I-370 | MS m/z 406.2 [M + H]⁺; ¹H NMR (400 MHz, CD₃OD) δ 7.80 (dd, J = 2.9, 1.2 |
| | Hz, 1H), 7.58 (d, J = 7.6 Hz, 1H), 7.39 (d, J = 6.5 Hz, 2H), 6.93 (dd, J = 3.8, 3.0 |
| | Hz, 1H), 6.47 (dd, J = 3.9, 1.2 Hz, 1H), 4.47 (s, 1H), 3.84 (d, J = 4.2 Hz, 3H), |
| | 3.15-3.10 (m, 1H), 2.71 (s, 1H), 2.34 (s, 3H), 2.09-2.07 (m, 2H), 1.89-1.84 |
| | (m, 1H), 1.77-1.74 (m, 1H), 1.73-1.62 (m, 1H), 1.62-1.59 (m, 1H). NH and |
| | OH peak not observed |
| I-371 | MS m/z 458.2 [M + H]⁺ .; ¹H NMR (400 MHz, DMSO-d6) δ 8.24 (s, 1H), 8.20 (d, |
| | J = 8.4 Hz, 1H), 8.01 (d, J = 2.0 Hz, 1H), 7.91 (d, J = 7.6 Hz, 1H), 7.35 (d, J = 8.0 |
| | Hz, 1H), 6.95 (t, J = 3.2 Hz, 1H), 6.39 (d, J = 3.6 Hz, 1H), 4.30(s, 1H), 3.14 (d, J = |
| | 6.0 Hz, 1H), 2.84 (d, J = 9.2 Hz, 1H), 2.40 (d, J = 5.2 Hz, 2H), 2.05 (d, J = 9.6 Hz, |
| | 1H), 1.95-1.91(m, 2H), 1.82-1.76 (m, 1H), 1.65-1.54 (m, 1H), 1.49-1.44(m, 1H), |
| | 1.02(t, J = 7.2 Hz, 3H). |
| I-372 | MS m/z 410.7 [M + H]⁺; ¹H NMR (400 MHz, METHANOL-d4) δ 7.91-7.77 |
| | (m, 1H), 7.12-7.00 (m, 2H), 7.00-6.90 (m, 1H), 6.59-6.48 (m, 1H), 4.56- |
| | 4.45 (m, 1H), 3.27-3.13 (m, 1H), 2.82-2.71 (m, 1H), 2.46-2.24 (m, 5H), 2.16- |
| | 2.07 (m, 1H), 1.97-1.86 (m, 1H), 1.85-1.56 (m, 2H); 1H (NH) is not |
| | observed. |
| I-373 | MS m/z 438.4 [M + H]⁺; ¹H NMR (CD₃OD) δ 8.10 (d, J = 8.5 Hz, 1H), 7.97 (d, |
| | J = 2.1 Hz, 1H), 7.27 (d, J = 4.0 Hz, 1H), 7.10 (t, J = 3.4 Hz, 1H), 6.88-6.94 (m, |
| | 2H), 4.44 (dt, J = 8.3, 4.3 Hz, 1H), 3.70-3.79 (m, 2H), 3.16-3.21 (m, 1H), 2.73- |
| | 2.81 (m, 1H), 2.58-2.72 (m, 2H), 2.39-2.51 (m, 2H), 2.03-2.10 (m, 1H), 1.86- |
| | 1.93 (m, 1H), 1.68-1.82 (m, 2H). OH and NH not observed. |
| I-376 | MS m/z 429.4[M + H]⁺; ¹H NMR (400 MHz, DMSO-d6) δ 7.99 (s, 1H), 7.87- |
| | 7.73 (m, 2H), 7.70 (s, 1H), 7.39 (t, J = 65.7 Hz, 1H), 7.37 (s, 1 H), 6.96 (s, 1H), |
| | 6.50 (s, 1H), 4.35-4.26 (m, 1H), 4.06 (d, J = 9.5 Hz, 1H), 3.83 (d, J = 10.3 Hz, |
| | 1H), 3.45-3.36 (m, 2H), 2.18-2.08 (m, 1H), 1.85-1.61 (m, 3H) |
| I-377 | MS m/z 368.5 [M + H]⁺; ¹H NMR (400 MHz, METHANOL-d₄) δ 7.98-7.79 |
| | (m, 2H), 7.24 (s, 1H), 7.12-6.97 (m, 1H), 6.64-6.46 (m, 2H), 4.52-4.33 |
| | (m, 1H), 3.83 (s, 3H), 3.43-3.40 (m, 1H), 3.04 (d, J = 1.6 Hz, 1H), 2.79 (d, |
| | J = 4.9 Hz, 2H), 2.55 (m, 2H), 2.25-2.08 (m, 1H), 1.96 (d, J = 1.3 Hz, 1H), |
| | 1.88-1.64 (m, 2H), 1.35-1.18 (m, 3H). NH and OH peak not observed |
| I-378 | MS m/z 442.2 [M + H]⁺; ¹H NMR (500 MHz, METHANOL-d4) δ 7.90-7.72 |
| | (m, 3H), 7.67 (s, 1H), 7.00-6.98 (m, 1H), 6.93 (t, J = 75 Hz, 1H) 6.57 (dd, J = |
| | 0.9, 4.0 Hz, 1H), 4.34-4.17 (m, 1H), 3.00-2.82 (m, 1H), 2.48-2.35 (m, 1H), |
| | 2.25-2.12 (m, 1H), 2.02-1.84 (m, 2H), 1.64-1.48 (m, 1H), 1.48-1.27 (m, |
| | 2H), 1.25-1.09 (m, 1H); 3H (NH) not observed |
| I-379 | MS m/z 412.5 [M + H]⁺; ¹H NMR (500 MHz, METHANOL-d4) δ 7.80 (s, 1H), |
| | 6.96 (s, 1H), 6.56 (s, 1H), 6.52 (s, 1H), 6.46 (d, J = 10.7 Hz, 1H), 4.62-4.54 |
| | (m, 1H), 3.93-3.78 (m, 3H), 3.47-3.37 (m, 1H), 3.21-3.10 (m, 2H), 3.01- |
| | 2.84 (m, 2H), 2.28-2.19 (m, 1H), 2.15-2.06 (m, 1H), 2.02-1.76 (m, 3H), |
| | 1.07-0.98 (m, 2H), 0.81-0.69 (m, 2H). 3H not observed (NH and 2 OH). |
| I-380 | MS m/z 382.5 [M + H]⁺; ¹H NMR (500 MHz, METHANOL-d4) δ 7.80 (s, 1H), |
| | 6.97 (s, 1H), 6.65-6.38 (m, 3H), 4.60-4.49 (m, 1H), 3.80-3.68 (m, 1H), 2.98- |
| | 2.87 (m, 2H), 2.81 (s, 3H), 2.29-2.19 (m, 1H), 2.17-2.08 (m, 1H), 1.99- |
| | 1.78 (m, 4H), 1.07-0.99 (m 2H), 0.81-0.70 (m, 2H). 2H not observed (NH |
| | and OH). |
| I-388 | MS m/z 415.1 [M + H]⁺; ¹H NMR (500 MHz, METHANOL-d4) δ 7.80 (dd, J = |
| | 2.8, 1.0 Hz, 1H), 7.72-7.68 (m, 1H), 7.66-7.61 (m, 1H), 7.58-7.54 (m, 1H), |
| | 6.88 (dd, J = 3.8, 3.1 Hz, 1H), 6.81 (t, J = 73.1 Hz, 1H), 6.47 (dd, J = 3.9, 1.0 |
| | Hz, 1H), 4.76-4.69 (m, 1H), 4.08-3.92 (m, 2H), 3.87-3.75 (m, 2H), 2.41- |
| | 2.29 (m, 1H), 2.14-2.02 (m, 1H). NH not observed. |
| I-389 | MS m/z 403.2 [M + H]⁺; ¹H NMR (500 MHz, METHANOL-d₄) δ 7.71 (dt, J = |
| | 3.1, 1.7 Hz, 1H), 7.68 (s, 1H), 7.65-7.61 (m, 1H), 7.57-7.54 (m, 1H), 6.89 |
| | (dd, J = 3.8, 2.9 Hz, 1H), 6.81 (t, J = 73.4 Hz, 1H), 6.46 (dd, J = 3.8, 1.1 Hz, |
| | 1H), 4.14-4.06 (m, 1H), 3.67-3.58 (m, 1H), 3.50-3.37 (m, 1H), 1.18 (d, J = |
| | 6.3 Hz, 3H). NH peak not observed. |
| I-392 | MS m/z 420.2 [M + H]⁺; ¹H NMR (400 MHz, CD₃OD) δ 7.81 (dd, J = 2.8 Hz, |
| | 1.2 Hz, 1H), 7.58 (d, J = 8.0 Hz, 1H), 7.41 (d, J = 7.2 Hz, 2H), 6.95 (t, J = 3.6 |
| | Hz, 1H), 6.51 (dd, J = 4.0 Hz, 1.2 Hz, 1H), 4.56-4.50 (m, 1H), 3.83 (s, 3H), |
| | 3.65-3.59 (m, 1H), 3.27-3.19 (m, 1H), 2.99-2.88 (m, 2H), 2.76-2.62 (m, |
| | 2H), 2.22-2.19 (m, 1H), 2.08-2.03 (m, 1H), 1.92-1.77 (m, 2H), 1.28 (t, J = |
| | 7.2 Hz, 3H). NH and OH not observed |
| I-393 | MS m/z 410.2 [M + H]⁺; ¹H NMR (400 MHz, DMSO-d6) δ 11.09 (s, 1H), 7.87 |
| | (t, J = 3.7 Hz, 1H), 7.58 (d, J = 8.0 Hz, 1H), 7.33 (d, J = 7.7 Hz, 1H), 7.30-7.18 |
| | (m, 2H), 6.91 (dd, J = 5.3, 3.3 Hz, 1H), 4.27 (s, 1H), 2.88 (m, 2H), 2.21 (s, 3H), |
| | 2.03-1.99(m, 1H), 1.96-1.85 (m, 1H), 1.81-1.69 (m, 1H), 1.66-1.35 (m, |
| | 2H), 1.23 (s, 1H). |
| I-394 | MS m/z 459.1 [M + H]⁺; ¹H NMR (400 MHz, DMSO-d6) δ 7.90 (t, J = 3.8 Hz, |
| | 1H), 7.83-7.71 (m, 2H), 7.65 (s, 1H), 7.45 (d, J = 7.6 Hz, 1H), 7.40 (t, J = 72.9 |
| | Hz, 1H), 6.93 (d, J = 3.4 Hz, 1H), 4.31 (s, 1H), 3.15-2.67 (m, 2H), 2.26 (s, |
| | 3H), 2.02-1.99 (m, 2H), 1. 80-1.76 (m, 1H), 1.71-1.34 (m, 2H), 1.24 (d, |
| | J = 3.4 Hz, 1H). |
| I-395 | MS m/z 397.3 [M + H]⁺; ¹H NMR (500 MHz, METHANOL-d4) δ 7.91 (br s, |
| | 1H), 7.13-7.04 (m, 2H), 7.00 (br s, 1H), 6.61 (br s, 1H), 4.37 (br s, 1H), 4.18 |
| | (br d, J = 8.7 Hz, 1H), 3.89 (br d, J = 11.1 Hz, 1H), 3.56-3.40 (m, 2H), 2.23 |
| | (br s, 1H), 1.90-1.76 (m, 3H); 2H (OH and NH) wasn't observed |
| I-399 | MS m/z 393.4 [M + H]⁺; ¹H NMR (500 MHz, DMSO-d6) δ 8.19-8.06 (m, 2H), |
| | 7.79-7.62 (m, 1H), 7.31-7.25 (m, 2H), 7.22 (d, J = 3.5 Hz, 1H), 7.13-7.08 |
| | (m, 1H), 4.85-4.71 (m, 1H), 4.24-4.09 (m, 1H), 3.61-3.50 (m, 1H), 2.32- |
| | 2.24 (m, 1H), 2.07-1.99 (m, 1H), 1.92-1.83 (m, 1H), 1.81-1.70 (m, 1H), |
| | 1.45-1.22 (m, 3H), 1.19-1.06 (m, 1H). 1H not observed (OH). |
| I-400 | MS m/z 407.4 [M + H]⁺; ¹H NMR (500 MHz, DMSO-d6) δ 8.14 (d, J = 8.5 Hz, |
| | 1H), 8.06 (s, 1H), 7.68 (d, J = 7.5 Hz, 1H), 7.31-7.24 (m, 2H), 7.20 (d, J = 3.7 |
| | Hz, 1H), 7.10 (t, J = 3.4 Hz, 1H), 4.26-4.16 (m, 1H), 3.30-3.23 (m, 4H), |
| | 2.46-2.41 (m, 1H), 2.05 (br d, J = 10.1 Hz, 2H), 1.85-1.78 (m, 1H), 1.41- |
| | 1.27 (m, 3H), 1.13-1.01 (m, 1H). 1H not observed (OH). |
| I-401 | MS m/z 379.4 [M + H]⁺; ¹H NMR (500 MHz, DMSO-d6) δ 8.14-8.08 (m, 2H), |
| | 7.58 (d, J = 7.3 Hz, 1H), 7.31-7.25 (m, 2H), 7.18 (d, J = 3.8 Hz, 1H), 7.10 (t, |
| | J = 3.4 Hz, 1H), 4.34-4.24 (m, 1H), 4.06 (dd, J = 10.6, 3.0 Hz, 1H), 3.83 (d, |
| | J = 11.1 Hz, 1H), 3.33-3.25 (m, 2H), 2.13 (d, J = 11.3 Hz, 1H), 1.83-1.60 (m, |
| | 3H). 1H not observed (OH). |
| I-402 | MS m/z 345.2, m/z347.2 [M + H]⁺; ¹H NMR (500 MHz, MeOD) δ 8.01-7.76 |
| | (m, 2H), 7.23 (d, J = 4.0 Hz, 1H), 7.07 (t, J = 3.6 Hz, 1H), 6.99 (d, J = 7.8 Hz, |
| | 2H), 4.31 (dt, J = 9.3, 4.9 Hz, 1H), 4.16 (dd, J = 10.8, 4.1 Hz, 1H), 3.89 (d, J = |
| | 10.8 Hz, 1H), 3.60-3.49 (m, 1H), 3.44 (t, J = 9.9 Hz, 1H), 2.23 (s, 1H), 1.82 |
| | (q, J = 12.1 Hz, 3H). NH and OH peak not observed. |
| I-403 | MS m/z 416.4, 418.3 [M + H]⁺. ¹H NMR (500 MHz, MeOD) δ 8.00-7.84 (m, |
| | 2H), 7.21 (d, J = 4.1 Hz, 1H), 7.05 (q, J = 3.1 Hz, 1H), 6.97 (d, J = 7.5 Hz, 2H), |
| | 4.37 (tt, J = 8.8, 3.8 Hz, 1H), 3.35 (s, 2H), 2.97-2.85 (m, 1H), 2.50 (t, J = 8.6 |
| | Hz, 1H), 2.45 (d, J = 2.7 Hz, 1H), 2.03 (q, J = 9.2 Hz, 1H), 1.93-1.73 (m, 2H), |
| | 1.66-1.54 (m, 1H), 1.34-1.30 (m, 1H), 1.28 (s, 3H), 1.22 (d, J = 15.7 Hz, |
| | 3H). NH and OH peaks not observed. |
| I-404 | MS m/z 386.3, 388.3 [M + H⁺]⁺; ¹H NMR (500 MHz, MeOD) δ 7.95-7.81 (m, |
| | 2H), 7.19 (d, J = 4.0 Hz, 1H), 7.14-7.04 (m, 1H), 6.99 (d, J = 7.1 Hz, 2H), |
| | 4.50 (s, 1H), 3.55 (d, J = 11.0 Hz, 1H), 3.28-3.22 (m, 1H), 3.22-3.10 (m, |
| | 1H), 2.78 (d, J = 9.9 Hz, 1H), 2.70 (d, J = 3.0 Hz, 1H), 2.21 (d, J = 12.1 Hz, |
| | 1H), 2.05 (d, J = 13.9 Hz, 1H), 1.94-1.82 (m, 1H), 1.74 (d, J = 11.9 Hz, 1H), |
| | 1.27 (dd, J = 6.6, 3.7 Hz, 6H). NH and OH peak not observed. |
| I-405 | MS m/z 433.30 [M + H]⁺. ¹H NMR (500 MHz, Methanol-d4) δ 8.13 (d, J = 8.2 |
| | Hz, 1H), 7.91 (d, J = 1.2 Hz, 1H), 7.27 (d, J = 1.8 Hz, 1H), 7.25-7.22 (m, 2H), |
| | 7.09 (dd, J = 4.1, 3.0 Hz, 1H), 4.44 (p, J = 8.0 Hz, 1H), 3.15-3.07 (m, 2H), |
| | 2.52-2.40 (m, 2H). OH and NH not observed. |
| I-406 | MS m/z 379.4 [M + H⁺]⁺; ¹H NMR (500 MHz, Methanol-d4) δ 8.16 (d, J = 8.2 |
| | Hz, 1H), 7.92 (s, 1H), 7.30-7.21 (m, 3H), 7.12-7.05 (m, 1H), 4.23 (p, J = 8.0 |
| | Hz, 1H), 2.71-2.62 (m, 2H), 2.33-2.23 (m, 2H), 1.45 (s, 3H). NH and OH |
| | not observed |
| I-407 | MS m/z 363.4 [M + H]⁺; ¹H NMR (CD₃OD) δ 7.81-7.85 (m, 1H), 7.59 (d, J = 7.6 |
| | Hz, 1H), 7.55 (s, 1H), 7.49 (d, J = 7.8 Hz, 1H), 6.96 (t, J = 3.1 Hz, 1H), 6.50 (d, |
| | J = 3.8, Hz, 1H), 4.49 (br s, 1H), 3.85 (s, 3H), 3.14-3.29 (m, 1H), 2.71-2.84 (m, |
| | 1H), 2.28-2.43 (m, 5H), 2.08-2.16 (m, 1H), 1.85-1.95 (m, 1H), 1.74-1.83 (m, |
| | 1H), 1.57-1.70 (m, 1H). NH not observed. |
| I-412 | MS m/z 442.4 [M + H]⁺; ¹H NMR (400 MHz, DMSO-d6) δ 8.16-8.04 (m, 2H), |
| | 7.56 (d, J = 7.6 Hz, 1H), 7.31-7.23 (m, 2H), 7.18 (s, 1H), 7.10 (s, 1H), 6.18 |
| | (d, J = 56.4 Hz, 1H), 4.40-4.25 (m, 1H), 3.27-3.19 (m, 1H), 2.95-2.87 (m, |
| | 1H), 2.87-2.75 (m, 2H), 2.29-2.14 (m, 2H), 2.10-1.99 (m, 1H), 1.83-1.70 |
| | (m, 1H), 1.68-1.37 (m, 2H) |
| I-414 | MS m/z 424.5 [M + H]⁺; ¹H NMR (400 MHz, METHANOL-d4) δ 7.90-7.78 |
| | (m, 1H), 7.11-7.02 (m, 2H), 7.00-6.92 (m, 1H), 6.59-6.52 (m, 1H), 4.62- |
| | 4.44 (m, 1H), 3.51-3.39 (m, 1H), 3.07-2.91 (m, 1H), 2.81-2.63 (m, 2H), |
| | 2.57-2.39 (m, 2H), 2.23-2.08 (m, 1H), 2.03-1.90 (m, 1H), 1.90-1.66 (m, |
| | 2H), 1.28-1.17 (m, 3H); NH and OH not observed |
| I-415 | MS m/z 440.3 [M + H]⁺; ¹H NMR (400 MHz, METHANOL-d4) δ 7.95 (br s, |
| | 1H), 8.08-7.80 (m, 1H), 7.24-6.97 (m, 3H), 6.85-6.48 (m, 1H), 4.76-4.58 |
| | (m, 1H), 4.24-4.00 (m, 1H), 4.00-3.84 (m, 2H), 3.82-3.57 (m, 1H), 3.19- |
| | 2.84 (m, 2H), 2.42-1.78 (m, 5H); 1H was overlapped with solvent peak. 3H |
| | (2OH and NH) not observed |
| I-416 | MS m/z 428.3 [M + H]⁺; ¹H NMR (400 MHz, METHANOL-d4) δ 8.36 (br s, |
| | 1H), 7.40 (br s, 2H), 7.33-7.13 (m, 2H), 5.47-5.22 (m, 1H), 4.10-3.79 (m, |
| | 2H), 3.57-3.36 (m, 1H), 3.27-3.12 (m, 1H), 3.04 (br s, 3H), 2.79-2.62 (m, |
| | 1H), 2.33-2.06 (m, 1H); 1H was overlapped with solvent peak, 2H (OH & |
| | NH) not observed |
| I-417 | MS m/z 468.4 [M + H]⁺; ¹H NMR (400 MHz, METHANOL-d4) δ 8.00-7.66 |
| | (m, 1H), 7.24-6.86 (m, 3H), 6.74-6.39 (m, 1H), 4.76-4.52 (m, 1H), 3.99- |
| | 3.74 (m, 1H), 3.61-3.40 (m, 1H), 3.05-2.90 (m, 4H), 2.39-1.95 (m, 3H), 1.94- |
| | 1.73 (m, 1H), 1.35 (br s, 6H). OH and NH not observed. |
| I-420 | MS m/z 395.2 [M − H]−; ¹H NMR (400 MHz, METHANOL-d4) δ 8.09-7.77 (m, |
| | 1H), 7.33-6.88 (m, 3H), 6.83-6.55 (m, 1H), 4.42-4.11 (m, 1H), 2.67 (br s, |
| | 2H), 2.31 (br s, 2H), 1.45 (br s, 3H); NH and OH not observed |
| I-421 | MS m/z 343.3 [M + H]⁺; ¹H NMR (400 MHz, METHANOL-d4) δ 7.96-7.77 |
| | (m, 1H), 7.07-6.90 (m, 1H), 6.73-6.45 (m, 3H), 4.42-4.31 (m, 1H), 4.24- |
| | 4.11 (m, 1H), 3.93-3.83 (m, 1H), 3.57-3.38 (m, 2H), 2.46-2.16 (m, 4H), |
| | 1.91-1.70 (m, 3H). 2 H not observed (NH and OH). |
| I-422 | MS m/z 357.3 [M + H]⁺; ¹H NMR (400 MHz, METHANOL-d4) δ 8.01-7.70 |
| | (m, 1H), 7.13-6.88 (m, 1H), 6.83-6.45 (m, 3H), 4.32-4.16 (m, 1H), 3.81- |
| | 3.67 (m, 1H), 2.54-2.23 (m, 4H), 2.23-2.04 (m, 1H), 2.04-1.93 (m, 1H), |
| | 1.93-1.80 (m, 1H), 1.54-1.17 (m, 4H). 3H not observed (NH and 2 OH). |
| I-423 | MS m/z 369.3 [M + H]⁺; ¹H NMR (400 MHz, METHANOL-d4) δ 7.95-7.75 |
| | (m, 1H), 7.05-6.88 (m, 1H), 6.66-6.35 (m, 3H), 4.43-4.24 (m, 1H), 4.23- |
| | 4.13 (m, 1H), 3.93-3.80 (m, 1H), 3.58-3.38 (m, 2H), 2.30-2.15 (m, 1H), |
| | 1.95-1.72 (m, 4H), 1.09-0.96 (m, 2H), 0.81-0.67 (m, 2H). 2H not observed |
| | (NH and OH). |
| I-424 | MS m/z 383.3 [M + H]⁺; ¹H NMR (400 MHz, METHANOL-d4) δ 7.90-7.78 |
| | (m, 1H), 7.04-6.90 (m, 1H), 6.70-6.38 (m, 3H), 4.31-4.13 (m, 1H), 3.83- |
| | 3.66 (m, 1H), 2.51-2.35 (m, 1H), 2.22-2.06 (m, 1H), 2.04-1.78 (m, 3H), |
| | 1.55-1.19 (m, 4H), 1.08-0.95 (m, 2H), 0.85-0.66 (m, 2H). 3H not observed |
| | (NH and 2 OH). |
| I-426 | MS m/z 426.3 [M + H+]⁺; ¹H NMR (400 MHz, MeOD) δ 8.10 (s, 1H), 7.99 (d, |
| | J = 8.3 Hz, 1H), 7.90 (s, 2H), 7.06 (t, J = 56 Hz, 1H), 7.01 (s, 1H), 6.61 (d, J = |
| | 3.8 Hz, 1H), 4.55-4.42 (m, 1H), 3.23-3.10 (m, 1H), 2.77 (s, 1H), 2.47-2.27 (m, |
| | 5H), 2.18-2.03 (m, 1H), 1.98-1.84 (m, 1H), 1.84-1.71 (m, 1H), 1.71-1.55 |
| | (m, 1H). NH peak not observed. |
| I-427 | MS m/z 413.3. [M + H⁺]⁺; ¹H NMR (400 MHz, MeOD) δ 8.09 (s, 1H), 7.98 (d, |
| | J = 8.3 Hz, 1H), 7.95-7.85 (m, 2H), 7.08 (t, J = 56 Hz, 1H), 6.99 (t, J = 3.3 Hz, |
| | 1H), 6.64-6.54 (m, 1H), 4.27 (p, J = 8.3 Hz, 1H), 2.66 (m, J = 2.1 Hz, 2H), |
| | 2.35-2.21 (m, 2H), 1.44 (s, 3H). NH and OH peaks not observed. |
| I-428 | MS m/z 406.6 [M + H]⁺; ¹H NMR (500 MHz, METHANOL-d4) δ 7.88 (br s, |
| | 1H), 7.10-7.03 (m, 2H), 7.03-6.66 (m, 2H), 6.53 (d, J = 3.5 Hz, 1H), 4.72- |
| | 4.47 (m, 1H), 3.96-3.74 (m, 1H), 3.53-3.38 (m, 1H), 3.22-3.01 (m, 2H), |
| | 2.93 (s, 3H), 2.50 (s, 3H), 2.32-2.14 (m, 2H), 2.04-1.82 (m, 2H); 1H (NH) |
| | wasn't observed |
| I-430 | MS m/z 480.3 [M + H]⁺; ¹H NMR (400 MHz, METHANOL-d4) δ 7.86 (br s, |
| | 1H), 7.15-7.05 (m, 2H), 7.00 (br s, 1H), 6.58 (br s, 1H), 4.60 (br s, 1H), 4.06 |
| | (br s, 2H), 3.96-3.75 (m, 1H), 3.54-3.38 (m, 3H), 3.02-2.81 (m, 2H), 2.35- |
| | 2.20 (m, 1H), 2.19-1.67 (m, 8H); 2H (OH and NH) wasn't observed |
| I-431 | MS m/z 406.6 [M + H]⁺; ¹H NMR (500 MHz, ACETONITRILE-d3) δ = 8.07- |
| | 7.77 (m, 1H), 7.27-6.83 (m, 2H), 6.76 (br s, 1H), 6.27 (br s, 1H), 4.72 (br s, |
| | 1H), 4.47 (br s, 1H), 3.37-2.68 (m, 4H), 2.60-2.33 (m, 3H), 2.01 (br s, 4H), |
| | 1.84-1.64 (m, 3H); 1H (OH or NH) wasn't observed |
| I-432 | MS m/z 376.2, 378.2 [M + H]⁺; ¹H NMR (400 MHz, DMSO) δ 7.93 (s, 1H), 7.25 |
| | (s, 1H), 6.98 (d, J = 9.3 Hz, 1H), 6.88 (d, J = 11.9 Hz, 2H), 6.35 (s, 1H), 4.32- |
| | 4.28 (m, 1H), 3.16 (d, J = 3.9 Hz, 1H), 3.12-3.07 (m, 1H), 2.76-2.71 (m, |
| | 1H), 2.24 (s, 3H), 2.00-1.96 (m, 3H), 1.78-1.74 (m, 1H), 1.61 (d, J = 12.9 |
| | Hz, 1H), 1.43 (d, J = 12.7 Hz, 1H). |
| I-433 | MS m/z 390.1, 392.1 [M + H⁺]⁺; ¹H NMR (400 MHz, MeOD) δ 7.80 (s, 1H), |
| | 7.01-6.90 (m, 1H), 6.81 (d, J = 7.4 Hz, 2H), 6.53 (d, J = 4.0 Hz, 1H), 4.55- |
| | 4.44 (m, 1H), 3.35 (s, 1H), 2.99-2.84 (m, 1H), 2.65 (d, J = 7.8 Hz, 2H), 2.45- |
| | 2.30 (m, 2H), 2.21-2.08 (m, 1H), 1.99-1.88 (m, 1H), 1.79 (d, J = 11.9 Hz, |
| | 1H), 1.68 (d, J = 11.5 Hz, 1H), 1.19 (t, J = 7.3 Hz, 3H). NH and OH peak not |
| | observed. |
| I-434 | MS m/z 363.2, 365.2 [M + H]⁺; ¹H NMR (400 MHz, MeOD) δ 8.14 (s, 1H), 7.90 |
| | (s, 1H), 7.01 (s, 1H), 6.86 (d, J = 7.1 Hz, 1H), 6.67 (s, 1H), 4.24 (p, J = 8.1 Hz, |
| | 1H), 2.66 (t, J = 9.2 Hz, 2H), 2.28 (t, J = 9.8 Hz, 2H), 1.44 (s, 3H). NH and OH |
| | peak not observed. |
| I-437 | MS m/z 369.5 [M + H]⁺; ¹H NMR (400 MHz, CD₃OD) δ 8.29 (br s, 1H), 7.74 (br |
| | d, J = 7.6 Hz, 1H), 7.36 (br s, 2H), 6.99-7.08 (m, 2H), 4.17-4.28 (m, 1H), 3.65- |
| | 3.77 (m, 1H), 3.55-3.64 (m, 1H), 1.31 (d, J = 6.3 Hz, 3H). OH and NH not |
| | observed. |
| I-438 | MS m/z 383.3 [M + H]⁺; ¹H NMR (CD₃OD) δ 8.07 (br d, J = 8.6 Hz, 1H), 7.91 (br |
| | s, 1H), 7.26 (br s, 1H), 7.11 (br s, 1H), 6.87-6.94 (m, 2H), 3.72 (s, 2H), 1.29- |
| | 1.38 (m, 6H). OH and NH not observed. |
| I-440 | MS m/z 421.6 [M + H]⁺; ¹H NMR (CD₃OD) δ 7.67-7.81 (m, 3H), 7.60 (br s, |
| | 1H), 6.94 (t, J = 72 Hz, 1H), 6.83 (bs, 1H), 4.12-4.28 (m, 1H), 3.74 (br d, |
| | J = 13.5 Hz, 1H), 3.47-3.60 (m, 1H), 1.24-1.35 (m, 3H). NH not observed. |
| I-441 | MS m/z 426.6 [M + H]⁺; ¹H NMR (CD₃OD) δ 7.74 (br s, 1H), 7.62 (br d, J = 8.3 |
| | Hz, 1H), 6.81-6.90 (m, 3H), 4.44 (br s, 1H), 3.13-3.25 (m, 1H), 2.66-2.90 (m, |
| | 1H), 2.25-2.47 (m, 5H), 2.09 (br s, 1H), 1.89 (br s, 1H), 1.70-1.84 (m, 1H), |
| | 1.50-1.70 (m, 1H) |
| I-447 | MS m/z 466.3 [M + H]⁺; ¹H NMR (400 MHz, METHANOL-d4) δ = 8.06-7.66 |
| | (m, 1H), 7.35-6.83 (m, 3H), 6.61 (br dd, J = 2.8, 4.2 Hz, 1H), 4.58 (br s, 1H), |
| | 4.03 (br s, 2H), 3.88 (br s, 1H), 3.74 (br s, 3H), 3.29-3.13 (m, 1H), 2.95-2.71 |
| | (m, 2H), 2.42-2.19 (m, 2H), 2.19-2.02 (m, 2H), 1.98-1.75 (m, 2H); 2H (NH |
| | and OH) not observed |
| I-448 | MS m/z 379.05 [M + H⁺]⁺; ¹H NMR (500 MHz, Methanol-d4) δ 8.15 (d, J = 8.1 |
| | Hz, 1H), 7.90 (d, J = 2.9 Hz, 1H), 7.29-7.20 (m, 3H), 7.10-7.04 (m, 1H), |
| | 4.55 (p, J = 7.2 Hz, 1H), 4.40-4.33 (m, 1H), 2.54-2.46 (m, 1H), 2.28-2.20 |
| | (m, 1H), 2.06-1.73 (m, 4H). OH and NH not observed. |
| I-449 | MS m/z 379.40 [M + H⁺]⁺; ¹H NMR (500 MHz, Methanol-d4) δ 8.16 (d, J = 8.2 |
| | Hz, 1H), 7.92 (d, J = 2.9 Hz, 1H), 7.30-7.21 (m, 3H), 7.10-7.04 (m, 1H), |
| | 4.79 (p, J = 7.2 Hz, 1H), 4.48-4.41 (m, 1H), 2.49-2.38 (m, 1H), 2.28-2.21 |
| | (m, 1H), 2.19-2.09 (m, 1H), 2.05-1.96 (m, 1H), 1.80-1.67 (m, 2H). OH and |
| | NH not observed. |
| I-450 | MS m/z 407.6 [M + H⁺]⁺; ¹H NMR (500 MHz, Methanol-d4) δ 8.19 (d, J = 8.2 |
| | Hz, 1H), 7.94 (s, 1H), 7.35-7.22 (m, 3H), 7.14-7.05 (m, 1H), 4.59-4.45 (m, |
| | 1H), 2.30-2.16 (m, 2H), 2.02-1.88 (m, 1H), 1.80-1.65 (m, 2H), 1.61-1.50 |
| | (m, 1H), 1.47-1.23 (m, 6H). OH and NH not observed. |
| I-454 | MS m/z 436.2 [M + H⁺]⁺; ¹H NMR (400 MHz, CD₃OD) δ 7.74 (dd, J = 2.8 Hz, |
| | 1.1 Hz, 1H), 7.50 (d, J = 8.2 Hz, 1H), 7.31 (d, J = 6.4 Hz, 2H), 6.90-6.81 (m, |
| | 1H), 6.39 (dd, J = 3.9 Hz, 1.1 Hz, 1H), 4.41 (dd, J = 23.0 Hz, 19.1 Hz, 1H), 3.73 |
| | (s, 3H), 3.68-3.59 (m, 2H), 3.12-3.04 (m, 1H), 2.73-2.63 (m, 1H), 2.60-2.51 |
| | (m, 2H), 2.45-2.30 (m, 2H), 2.05-1.90 (m, 1H), 1.85-1.75 (m, 1H), 1.76- |
| | 1.58(m, 2H). OH and NH not observed. |
| I-456 | MS m/z 372.5 [M + H]⁺; ¹H NMR (400 MHz, METHANOL-d4) δ 7.85-7.72 |
| | (m, 1H), 6.95 (s, 1H), 6.58 (s, 1H), 6.36 (s, 2H), 5.49 (s, 1H), 4.51 (m, 1H), |
| | 3.82 (m, 3H), 3.60-3.46 (m, 1H), 3.17-3.02 (m, 1H), 2.80-2.68 (m, 2H), |
| | 2.65 (s, 3H), 2.17 (m, 1H), 2.04 (m, 1H), 1.95-1.69 (m, 2H). OH and NH not |
| | observed |
| I-457 | MS m/z 402.6 [M + H]⁺; ¹H NMR (400 MHz, METHANOL-d4) δ 8.58-8.45 |
| | (m, 1H), 7.87-7.75 (m, 1H), 6.99-6.90 (m, 1H), 6.63-6.52 (m, 1H), 6.36 (s, |
| | 2H), 4.56-4.45 (m, 1H), 3.82 (s, 3H), 3.80 (s, 2H), 3.56-3.42 (m, 1H), 3.11- |
| | 3.00 (m, 1H), 2.94-2.78 (m, 2H), 2.73-2.58 (m, 2H), 2.21-2.07 (m, 1H), |
| | 2.04-1.92 (m, 1H), 1.91-1.68 (m, 2H). NH and OH peak not observed. |
| I-458 | MS m/z 415.5 [M + H]⁺; ¹H NMR (400 MHz, METHANOL-d₄) δ 8.32 (s, 1H, |
| | formic acid proton), 7.96-7.86 (m, 1H), 7.84-7.77 (m, 1H), 7.77-7.70 (m, |
| | 1H), 7.66 (s, 1H), 6.98 (d, J = 2.6 Hz, 1H), 6.90 (t, J = 72 Hz 1H), 6.57 (m, |
| | 1H), 4.27-4.01 (m, 2H), 4.01-3.81 (m, 2H), 2.61-2.36 (m, 1H), 2.30-2.02 |
| | (m, 1H). 1CH signal overlapped with solvent. NH and OH peak not observed. |
| I-459 | MS m/z 365.4 [M + H]⁺; ¹H NMR (400 MHz, METHANOL-d4) δ 8.14 (m, 1H), |
| | 7.96 (s, 1H), 7.33-7.19 (m, 3H), 7.08 (s, 1H), 4.83-4.77 (m, 1H), 4.17-4.02 |
| | (m, 2H), 4.01-3.84 (m, 2H), 2.54-2.37 (m, 1H), 2.25-2.09 (m, 1H). NH and |
| | OH peak were not observed. |
| I-462 | MS m/z 424.6 [M + H]⁺; ¹H NMR (400 MHz, METHANOL-d4) δ 7.82 (br s, |
| | 1H), 7.06-6.52 (m, 4H), 6.52 (br s, 1H), 5.31-5.08 (m, 1H), 3.80-3.65 (m, |
| | 1H), 3.58-3.43 (m, 1H), 3.09-2.84 (m, 1H), 2.82-2.66 (m, 4H), 2.49 (br s, |
| | 4H), 2.19-1.93 (m, 1H); NH not observed |
| I-463 | MS m/z 436.4 [M + H]⁺; ¹H NMR (400 MHz, METHANOL-d4) δ 7.85 (br s, |
| | 1H), 7.16-6.44 (m, 5H), 4.72-4.58 (m, 1H), 3.93 (br s, 3H), 3.68-3.53 (m, |
| | 1H), 3.19-2.97 (m, 2H), 2.57-2.41 (m, 3H), 2.39-1.74 (m, 5H); one H signal |
| | was overlapped with solvent peak; NH and OH not observed |
| I-464 | MS m/z 391.2 [M − H]⁻; ¹H NMR (400 MHz, METHANOL-d4) δ 7.88 (br s, 1H), |
| | 7.05 (br s, 2H), 6.98 (br s, 2H), 6.50 (br s, 1H), 4.45-4.31 (m, 1H), 4.31-4.08 |
| | (m, 1H), 3.90 (br d, J = 10.6 Hz, 1H), 3.61-3.41 (m, 2H), 2.48 (s, 3H), 2.33- |
| | 2.19 (m, 1H), 1.93-1.73 (m, 3H); NH wasn't observed |
| I-465 | MS m/z 391.2 [M − H]⁻; ¹H NMR (400 MHz, METHANOL-d4) δ 7.90-7.77 (m, |
| | 1H), 7.09-6.44 (m, 5H), 4.39-4.18 (m, 1H), 2.75-2.58 (m, 2H), 2.48 (s, 3H), |
| | 2.37-2.19 (m, 2H), 1.45 (s, 3H); NH and OH not observed. |
| I-466 | MS m/z 343.5 [M + H⁺]⁺; ¹H NMR (400 MHz, Methanol-d4) δ 7.97-7.69 (m, |
| | 1H), 7.06-6.88 (m, 1H), 6.76-6.43 (m, 3H), 4.34-4.16 (m, 1H), 2.77-2.59 |
| | (m, 2H), 2.49-2.13 (m, 5H), 1.43 (s, 3H). OH and NH not observed. |
| I-467 | MS m/z 369.50 [M + H⁺]⁺; ¹H NMR (400 MHz, Methanol-d4) δ 7.90-7.72 (m, |
| | 1H), 7.05-6.86 (m, 1H), 6.67-6.38 (m, 3H), 4.35-4.14 (m, 1H), 2.75-2.56 |
| | (m, 2H), 2.34-2.18 (m, 2H), 2.00-1.82 (m, 1H), 1.43 (s, 3H), 1.11-0.96 (m, |
| | 2H), 0.75 (s, 2H). OH and NH not observed. |
| I-469 | MS m/z 524.1 [M + H]⁺, ¹H NMR (400 MHz, DMSO-d6) δ 8.24 (s, 1H), 8.20 (d, |
| | J = 7.8 Hz, 1H), 7.99 (s, 1H), 7.90 (d, J = 7.8 Hz, 1H), 7.35 (d, J = 7.6 Hz, 1H), |
| | 6.95 (s, 1H), 6.67 (t, J = 77.2 Hz, 1H), 6.39 (s, 1H), 4.38-4.23 (m, 1H), 3.97- |
| | 3.89 (m, 2H), 3.18 (d, J = 9.5 Hz, 1H), 2.91-2.81 (m, 1H), 2.71-2.58 (m, |
| | 2H), 2.17-1.99 (m, 3H), 1.82-1.71 (m, 1H), 1.66-1.51 (m, 1H), 1.53-1.37 |
| | (m, 1H) |
| I-470 | MS m/z 502.1 [M + H]⁺; ¹H NMR (400 MHz, DMSO-d6) δ 8.24 (s, 1H), 8.20 (d, |
| | J = 8.3 Hz, 1H), 7.99 (s, 1H), 7.91 (d, J = 7.8 Hz, 1H), 7.29 (d, J = 7.4 Hz, 1H), |
| | 6.94 (s, 1H), 6.38 (s, 1H), 4.40-4.29 (m, 1H), 4.11 (s, 1H), 3.20 (d, J = 9.6 Hz, |
| | 1H), 2.91 (d, J = 10.3 Hz, 1H), 2.32-2.10 (m, 4H), 2.01 (d, J = 10.6 Hz, 1H), |
| | 1.81-1.68 (m, 1H), 1.68-1.54 (m, 1H), 1.54-1.36 (m, 1H), 1.10 (s, 6H). |
| I-471 | MS m/z 404.3 [M + H]⁺; ¹H NMR (400 MHz, MeOD) δ 7.81 (d, J = 3.1 Hz, 1H), |
| | 7.47 (dd, J = 8.7, 2.6 Hz, 1H), 6.95-6.86 (m, 3H), 6.77 (t, J = 76 Hz, 1H), 6.53 |
| | (d, J = 4.0 Hz, 1H), 4.47 (dq, J = 10.9, 5.0 Hz, 1H), 3.89 (d, J = 2.7 Hz, 3H), |
| | 3.22 (d, J = 11.2 Hz, 1H), 2.88-2.71 (m, 1H), 2.47-2.28 (m, 5H), 2.10 (d, J = |
| | 12.5 Hz, 1H), 1.90 (dd, J = 11.7, 6.4 Hz, 1H), 1.84-1.72 (m, 1H), 1.64 (d, J = |
| | 11.7 Hz, 1H). NH not observed |
| I-472 | MS/m/z 391.3 [M + H]⁺; ¹H NMR (400 MHz, MeOD) δ 7.79 (d, J = 3.1 Hz, |
| | 1H), 7.47 (d, J = 8.5 Hz, 1H), 6.96-6.85 (m, 3H), 6.77 (t, J = 76 Hz, 1H), 6.52 |
| | (d, J = 3.7 Hz, 1H), 4.25 (p, J = 8.3 Hz, 1H), 3.89 (d, J = 2.4 Hz, 3H), 2.65 (m, |
| | J = 4.3 Hz, 2H), 2.26 (t, J = 9.8 Hz, 2H), 1.43 (d, J = 2.9 Hz, 3H). NH and OH |
| | peak not observed. |
| I-473 | MS m/z 353.5 [M + H]⁺; ¹H NMR (400 MHz, METHANOL-d4) δ 8.21-8.09 |
| | (m, 1H), 7.87 (s, 1H), 7.33-7.18 (m, 3H), 7.08 (s, 1H), 4.26-4.12 (m, 1H), |
| | 3.79-3.66 (m, 1H), 3.59-3.47 (m, 1H), 1.38-1.20 (m, 3H). NH and 2 OH |
| | peaks not observed. |
| I-474 | MS m/z 365.4 [M + H]⁺; ¹H NMR (400 MHz, METHANOL-d4) δ 8.14 (d, J = |
| | 8.1 Hz, 1H), 7.97 (s, 1H), 7.24 (m, 3H), 7.08 (s, 1H), 4.84-4.76 (m, 1H), 4.14- |
| | 4.02 (m, 2H), 3.97-3.84 (m, 2H), 2.53-2.39 (m, 1H), 2.25-2.12 (m, 1H). |
| | NH and OH peaks not observed. |
| I-475 | MS m/z 476.4 [M + H]⁺; ¹H NMR (400 MHz, METHANOL-d4) δ 7.86 (br s, |
| | 1H), 7.06 (br s, 2H), 7.03-6.61 (m, 2H), 6.52 (br s, 1H), 4.63 (br s, 1H), 4.09 |
| | (br d, J = 10.9 Hz, 2H), 4.02-3.89 (m, 1H), 3.59-3.40 (m, 4H), 3.07 (br d, J = |
| | 10.9 Hz, 2H), 2.49 (br s, 3H), 2.36-2.16 (m, 2H), 2.16-1.74 (m, 6H); NH |
| | wasn't observed |
| I-476 | MS m/z 464.4 [M + H]⁺; ¹H NMR (400 MHz, METHANOL-d4) δ 7.85 (br s, |
| | 1H), 7.05 (br s, 2H), 7.02-6.59 (m, 2H), 6.51 (br s, 1H), 4.67 (br s, 1H), 3.98- |
| | 3.81 (m, 1H), 3.52 (br d, J = 10.6 Hz, 1H), 3.09-3.02 (m, 4H), 2.49 (br s, 3H), |
| | 2.33-2.15 (m, 1H), 2.15-1.99 (m, 2H), 1.94-1.74 (m, 1H), 1.35 (br s, 6H); |
| | OH and NH not observed |
| I-477 | MS m/z 352.3 [M + H]⁺; ¹H NMR (400 MHz, METHANOL-d4) δ 8.02 (br s, |
| | 1H), 7.08 (br s, 1H), 6.83-6.62 (m, 3H), 3.98-3.76 (m, 1H), 3.30-3.20 (m, |
| | 1H), 3.19-2.92 (m, 2H), 2.84-2.63 (m, 3H), 2.35 (br s, 4H), 2.14-2.01 (m, |
| | 5H), 1.95-1.76 (m, 1H); 1H was overlapped with solvent peak. NH and OH |
| | wasn't observed |
| I-479 | MS m/z 406.6 [M + H]⁺; ¹H NMR (400 MHz, Methanol-d4) δ 8.28 (m, 1H, |
| | formic acid proton), 8.20-8.02 (m, 1H), 7.96-7.80 (m, 1H), 7.37-7.15 (m, |
| | 3H), 7.13-6.97 (m, 1H), 4.64-4.47 (m, 1H), 3.85-3.66 (m, 1H), 3.43-3.35 |
| | (m, 1H), 3.20-3.02 (m, 2H), 2.97-2.74 (m, 2H), 2.35-2.06 (m, 2H), 2.04- |
| | 1.74 (m, 2H), 1.43-1.25 (m, 3H). OH and NH not observed |
| I-480 | MS m/z 420.6 [M + H]⁺; ¹H NMR (400 Hz, Methanol-d4) δ 8.48 (s, 1H, formic |
| | acid proton), 8.15-8.02 (m, 1H), 7.98-7.82 (m, 1H), 7.39-7.03 (m, 4H), |
| | 4.67-4.51 (m, 1H), 3.89-3.73 (m, 1H), 3.62-3.38 (m, 2H), 3.12-2.84 (m, |
| | 2H), 2.43-2.10 (m, 2H), 2.08-1.75 (m, 2H), 1.53-1.23 (m, 6H). OH and |
| | NH not observed |
| I-482 | MS m/z 406.3, 408.3 [M + H]⁺; ¹H NMR (400 MHz, MeOD) δ 7.91 (s, 1H), 7.08- |
| | 6.92 (m, 1H), 6.92-6.72 (m, 2H), 6.58 (s, 1H), 4.57-4.70 (m, 2H), 4.05- |
| | 3.81 (m, 3H), 3.66-3.53 (m, 1H), 3.25-2.97 (m, 2H), 2.38-1.79 (m, 5H). |
| | NH and OH peak not observed |
| I-483 | MS m/z 460.6 [M + H]⁺; ¹H NMR (400 MHz, Methanol-d4) δ 8.17-8.06 (m, |
| | 1H), 7.94-7.84 (m, 1H), 7.32-7.16 (m, 3H), 7.13-7.03 (m, 1H), 4.48-4.30 |
| | (m, 1H), 3.43-3.32 (m, 1H), 3.26-3.11 (m, 2H), 3.00-2.89 (m, 1H), 2.61- |
| | 2.45 (m, 2H), 2.19-2.07 (m, 1H), 1.90-1.70 (m, 2H), 1.66-1.50 (m, 1H). |
| I-487 | MS m/z 460.1 [M + H]⁺; ¹H NMR (400 MHz, METHANOL-d4) δ 8.53 (s, 1H, |
| | formic acid), 7.87 (s, 1H), 7.62 (d, J = 9.3 Hz, 1H), 7.53 (s, 1H), 7.00 (s, 1 H), |
| | 6.97 (t, J = 71.8 Hz, 1H), 6.53 (s, 1H), 4.64-4.46 (m, 1H), 3.46-3.36 (m, |
| | 1H), 3.06-2.92 (m, 1H), 2.64-2.45 (m, 5H), 2.23-2.10 (m, 1H), 2.08-1.92 |
| | (m, 1H), 1.92-1.62 (m, 2H). 1H not observed (NH) |
| I-488 | MS m/z 462.4 [M + H]⁺; ¹H NMR (400 MHz, METHANOL-d4) δ 7.85 (br s, |
| | 1H), 7.05 (br s, 2H), 7.01-6.58 (m, 2H), 6.49 (br s, 1H), 4.52 (br s, 1H), 4.01 |
| | (br s, 1H), 3.91 (br s, 2H), 3.84-3.67 (m, 1H), 3.64-3.39 (m, 2H), 3.20-2.99 |
| | (m, 1H), 2.74-2.57 (m, 2H), 2.48 (s, 3H), 2.34-2.15 (m, 2H), 2.04 (br d, J = |
| | 3.9 Hz, 2H), 1.94-1.70 (m, 2H); NH wasn't observed |
| I-489 | MS m/z 412.4 [M + H]⁺; ¹H NMR (400 MHz, METHANOL-d4) δ 7.67 (br s, |
| | 1H), 6.81 (br s, 1H), 6.58-6.35 (m, 3H), 4.33 (br s, 1H), 3.90-3.70 (m, 2H), |
| | 3.70-3.50 (m, 2H), 3.08-2.91 (m, 2H), 2.80-2.56 (m, 1H), 2.23 (br s, 3H), |
| | 2.19-2.09 (m, 2H), 2.00 (br s, 2H), 1.88-1.39 (m, 5H); 1H (NH) wasn't |
| | observed |
| I-491 | MS m/z 474.2 [M + H]⁺; ¹H NMR (400 MHz, CD₃OD) δ 8.12 (s, 1H), 8.05 (d, |
| | J = 8.1 Hz, 1H), 7.86 (d, J = 1.8 Hz, 1H), 7.78 (d, J = 8.0 Hz, 1H), 6.95-6.87 (m, |
| | 1H), 6.36 (d, J = 3.1 Hz, 1H), 4.44-4.42 (m, 1H), 3.72-3.61 (m, 2H), 3.09- |
| | 3.08 (m, 1H), 2.60-2.57(m, 1H), 2.55-2.50(m, 2H), 2.45-2.35 (m, 2H), 2.03- |
| | 1.94 (m, 1H), 1.85-1.76 (m, 1H), 1.74-1.57 (m, 2H). |
| I-492 | MS m/z 405.2 [M + H]⁺; ¹H NMR (400 MHz, CD₃OD) δ 8.17 (s, 1H), 8.11 (d, |
| | J = 7.6 Hz, 1H), 7.89-7.76 (m, 2H), 6.97 (dd, J = 4.0, 2.9 Hz, 1H), 6.42 (dd, J = |
| | 3.9, 1.2 Hz, 1H), 4.26-4.13 (m, 1H), 3.74 (dd, J = 13.8, 4.1 Hz, 1H), 3.55 (dd, |
| | J = 13.7, 7.6 Hz, 1H), 1.27 (d, J = 6.3 Hz, 3H). |
| I-493 | MS m/z 470.2 [M + H]⁺; ¹H NMR (400 MHz, DMSO-d6) δ 7.98-7.97 (m, 1H), |
| | 7.83-7.77(m, 2H), 7.70 (s, 1H), 7.37 (t, J = 73.6 Hz, 1H), 7.31 (d, J = 8.4 Hz, |
| | 1H), 6.95 (dd, J = 3.6 Hz, 2.8 Hz, 1H), 6.48 (dd, J = 4.0 Hz, 0.8 Hz, 1H), 4.29- |
| | 4.26 (m, 1H), 3.13-3.11 (m, 1H), 2.79-2.74 (m, 2H), 2.17-2.12 (m, 2H), |
| | 2.07-2.03 (m, 1H), 1.78-1.75 (m, 1H), 1.57-1.42 (m, 2H), 0.99 (d, J = 6.4 |
| | Hz, 6H). |
| I-494 | MS m/z 510.2 [M + H]⁺; ¹H NMR (400 MHz, DMSO-d6) δ 7.96 (dd, J = 2.8 Hz, |
| | 1.2 Hz, 1H), 7.82-7.77(m, 2H), 7.70 (s, 1H), 7.37 (t, J = 73.2 Hz, 1H), 7.35 (d, |
| | J = 8.0 Hz, 1H), 6.96 (dd, J = 3.6 Hz, 2.8 Hz, 1H), 6.49 (dd, J = 3.6 Hz, 0.8 Hz, |
| | 1H), 4.35-4.29 (m, 1H), 3.29-3.23 (m, 3H), 2.94-2.91 (m, 1H), 2.41-2.32 |
| | (m, 2H), 2.07-2.04 (m, 1H), 1.78-1.75 (m, 1H), 1.66-1.60 (m, 1H), 1.51- |
| | 1.44 (m, 1H). |
| I-495 | MS m/z 396.2 [M + H]⁺; ¹H NMR (400 MHz, CD₃OD) δ 7.71 (d, J = 2.0 Hz, |
| | 1H), 6.85 (t, J = 3.6 Hz, 1H), 6.45 (d, J = 6.8 Hz, 1H), 6.44 (s, 1H), 6.37 (dd, |
| | J = 1.6 Hz, J = 11.2 Hz, 1H), 4.42-4.38 (m, 1H), 3.15-3.13 (m, 1H), 2.72- |
| | 2.70 (m, 1H), 2.49-2.43 (m, 2H), 2.25-2.17 (m, 2H), 2.04-2.02 (m, 1H), |
| | 1.86-1.77 (m, 2H), 1.71-1.66 (m, 1H), 1.57-1.55 (m, 1H), 1.07 (t, J = 7.6 |
| | Hz, 3H), 0.97-0.92 (m, 2H), 0.68-0.64 (m, 2H). NH and OH not observed |
| I-496 | MS m/z 343.2 [M + H]⁺; ¹H NMR (400 MHz, DMSO-d6) δ 10.03 (s, 1H), 7.88 |
| | (d, J = 2.0 Hz, 1H), 7.63 (t, J = 5.2 Hz, 1H), 6.88 (t, J = 3.2 Hz, 1H), 6.54(s, 1H), |
| | 6.47(d, J = 10.8 Hz, 1H), 6.33 (d, J = 4.0 Hz, 1H), 4.97 (d, J = 4.4 Hz, 1H), 4.06- |
| | 4.02 (m, 1H), 3.58-3.51(m, 1H), 3.45-3.42(m, 1H), 1.95-1.89 (m, 1H), |
| | 1.15(d, J = 6.4 Hz, 3H), 1.01-0.97 (m, 2H), 0.72-0.69 (m, 2H). |
| I-497 | MS m/z 410.2, 412.2 [M + H]⁺; ¹H NMR (400 MHz, DMSO-d6) δ 8.22 (s, 1H, |
| | formic acid proton), 8.00 (t, J = 2.5 Hz, 2H), 7.89 (dd, J = 8.3, 1.9 Hz, 1H), 7.66 |
| | (d, J = 8.3 Hz, 1H), 7.33 (s, 1H), 6.93 (t, J = 3.2 Hz, 1H), 6.34 (dd, J = 3.8, 0.9 Hz, |
| | 1H), 4.31 (s, 1H), 3.09 (d, J = 7.5 Hz, 1H), 2.74 (d, J = 10.6 Hz, 1H), 2.24 (s, |
| | 3H), 2.03-1.93 (m, 3H), 1.79-1.76 (m, 1H), 1.63-1.60 (m, 1H), 1.45-1.41 |
| | (m, 1H). |
| I-499 | MS m/z 317.2 [M + H]⁺; ¹H NMR (400 MHz, CD₃OD) δ 7.74 (d, J = 1.8 Hz, 1H), |
| | 6.93(t, J = 4.0 Hz, 1H), 6.61 (s, 1H), 6.57 (dd, J = 10.6, 0.6 Hz, 1H), 6.54-6.51 |
| | (m, 1H), 4.25-4.10(m, 1H), 3.71 (dd, J = 13.8, 4.1 Hz, 1H), 3.59-3.48 (m, 1H), |
| | 2.34 (s, 3H), 1.27 (d, J = 6.3 Hz, 3H). NH and OH not observed. |
| I-500 | MS m/z 343.2 [M + H]⁺; ¹H NMR (400 MHz, DMSO-d6) δ 9.99 (s, 1H), 7.88 (d, |
| | J = 1.2 Hz, 1H), 7.63 (t, J = 5.6 Hz, 1H), 6.88 (t, J = 3.6 Hz, 1H), 6.5 3 (s, 1H), |
| | 6.47(d, J = 10.8 Hz, 1H), 6.33 (d, J = 4.0 Hz, 1H), 4.9 6 (d, J = 4.8 Hz, 1H), 4.08- |
| | 4.03 (m, 1H), 3.58-3.52 (m, 1H), 3.49-3.39 (m, 1H), 1.95-1.88(m, 1H), |
| | 1.15 (d, J = 6.0 Hz, 3H), 1.01-0.97 (m, 2H), 0.73-0.69 (m, 2H). |
| I-502 | MS m/z 390.5 [M + H]⁺; ¹H NMR (400 MHz, METHANOL-d4) δ 8.45 (br s, |
| | 1H), 7.83-7.72 (m, 1H), 7.00-6.90 (m, 1H), 6.64-6.55 (m, 1H), 6.36 (s, |
| | 2H), 5.14-4.92 (m, 1H), 4.83-4.72 (m, 1H), 3.82 (s, 3H), 3.41-3.34 (m, |
| | 1H), 3.18-3.06 (m, 1H), 2.45 (m, 5H), 2.38-2.27 (m, 1H), 2.04-1.80 (m, |
| | 1H). NH and OH not seem |
| I-505 | MS m/z 406.5, 408.5 [M + H]⁺; ¹H NMR (400 MHz, METHANOL-d4) δ 7.88 (s, |
| | 1H), 7.83 (s, 1H), 7.73-7.61 (m, 2H), 7.00 (s, 1 H), 6.94 (d, J = 55.4 Hz, 2H), |
| | 6.60 (s, 1H), 4.58-4.44 (m, 1H), 3.59-3.42 (m, 1H), 3.16-2.99 (m, 1H), |
| | 2.89-2.72 (m, 2H), 2.70-2.46 (m, 2H), 2.27-2.12 (m, 1H), 2.05-1.94 (m, |
| | 1H), 1.94-1.65 (m, 2H), 1.33-1.16 (m, 3H). NH and OH not observed |
| I-506 | MS m/z 422.6, 424.6 [M + H]⁺; ¹H NMR (400 MHz, METHANOL-d4) δ 7.88 (s, |
| | 1H), 7.84 (s, 1H), 7.74-7.62 (m, 2H), 7.01 (s, 1 H), 6.98 (t, J = 55.0 Hz, 1H), |
| | 6.62 (s, 1H), 4.63-4.53(m, 1H), 3.92-3.82 (m, 2H), 3.81-3.71 (m, 1H), 3.44- |
| | 3.34 (m, 1H), 3.22-3.06 (m, 2H), 3.06-2.81 (m, 2H), 2.27-2.17 (m, 1H), |
| | 2.17-2.05 (m, 1H), 2.03-1.75 (m, 2H). 2H not observed (NH, OH). |
| I-507 | MS m/z 416.2 [M + H]⁺; ¹H NMR (400 MHz, DMSO-d6) δ 7.95 (s, 1H), 7.56 (d, |
| | J = 7.9 Hz, 1H), 7.52 (s, 1H), 7.37-7.30 (m, 2H), 7.03 (t, J = 55.5 Hz, 1H), |
| | 6.96 (s, 1H), 6.51 (br s, 1H), 5.00 (d, J = 48.0 Hz, 1H), 4.68-4.55 (m, 1H), |
| | 3.08 (br d, J = 8.3 Hz, 1H), 3.03-2.80 (m, 1H), 2.33-2.19 (m, 5H), 2.17- |
| | 1.96 (m, 2H), 1.88-1.63 (m, 1H), 1.09-1.02 (m, 2H), 0.83-0.75 (m, 2H) |
| I-508 | MS m/z 412.6 [M + H]⁺; ¹H NMR (400 MHz, METHANOL-d4) δ 7.86 (s, 1H), |
| | 7.54 (s, 1H), 7.51 (d, J = 8.8 Hz, 1H), 7.36 (d, J = 7.9 Hz, 1H), 7.00 (s, 1H), |
| | 6.86 (t, J = 56.0 Hz, 1H), 6.60 (s, 1H), 4.63-4.48 (m, 1H), 3.98-3.76 (m, |
| | 1H), 3.60-3.43 (m, 1H), 3.28-3.19 (m, 2H), 3.14-2.86 (m, 2H), 2.33-2.15 |
| | (m, 2H), 2.14-2.03 (m, 1H), 2.04-1.80 (m, 2H), 1.37 (t, J = 7.2 Hz, 3H), 1.11 |
| | (d, J = 6.9 Hz, 2H), 0.86-0.79 (m, 2H). 1H not observed (NH). |
| I-509 | MS m/z 428.6 [M + H]⁺; ¹H NMR (400 MHz, METHANOL-d4) δ 7.85 (s, 1H), |
| | 7.54 (s, 1H), 7.51 (d, J = 8.0 Hz, 1H), 7.35 (d, J = 8.9 Hz, 1H), 6.99 (s, 1 H), |
| | 6.87 (t, J = 56.3 Hz, 1H), 6.59 (s, 1H), 4.63-4.51 (m, 1H), 3.92-3.84 (m, |
| | 2H), 3.83-3.73 (m, 1H), 3.45-3.36 (m, 1H), 3.21-3.09 (m, 2H), 3.02-2.80 |
| | (m, 2H), 2.28-2.16 (m, 1H), 2.16-2.04 (m, 2H), 2.03-1.78 (m, 2H), 1.11 (d, |
| | J = 7.0 Hz, 2H), 0.85-0.78 (m, 2H). 2H not observed (NH, OH). |
| I-510 | MS m/z 432.6 [M + H]⁺; ¹H NMR (400 MHz, METHANOL-d4) δ 8.26 (s, 1H, |
| | formic acid), 7.86 (s, 1H), 6.99 (s, 1H), 6.66 (s, 1H), 6.53 (s, 1H), 6.48 (d, J = |
| | 11.6 Hz, 1H), 6.02 (t, J = 55.9 Hz, 1H), 4.45-4.34 (m, 1H), 2.94-2.76 (m, |
| | 3H), 2.40 (t, J = 9.88 Hz, 2H), 2.17-2.06 (m, 1H), 1.98-1.52 (m, 5H), 1.04 |
| | (d, J = 7.0 Hz, 2H), 0.78-0.71 (m, 2H). 2H not observed (NH and OH). |
| I-511 | MS m/z 406.6 [M + H]⁺; ¹H NMR (400 MHz, METHANOL-d4) δ 8.22 (br s, |
| | 1H, formic acid), 7.95(s, 1H), 7.06 (s, 1H), 6.77 (s, 1H), 6.73-6.49 (m, 2H), |
| | 6.02 (t, J = 57.7 Hz, 1H), 4.48-4.33 (m, 1H), 2.95-2.76 (m, 4H), 2.46-2.23 |
| | (m, 5H), 2.17-2.06 (m, 1H), 1.89-1.67 (m, 2H), 1.67-1.50 (m, 1H). 2Hs not |
| | observed (NH and OH). |
| I-513 | MS m/z 375.3 [M + H]⁺; ¹H NMR (400 MHz, MeOD) δ 7.78 (s, 1H), 6.92 (t, J = |
| | 3.4 Hz, 1H), 6.63-6.47 (m, 1H), 6.42-6.27 (m, 2H), 4.53-4.39 (m, 1H), |
| | 3.23-3.06 (m, 1H), 2.82-2.62 (m, 1H), 2.42-2.17 (m, 5H), 2.16-2.02 (m, |
| | 1H), 1.95-1.84 (m, 1H), 1.82-1.70 (m, 1H), 1.70-1.53 (m, 1H). 1 OH and 1 |
| | NH signals are not observed. |
| I-516 | MS m/z 514.3 [M + H]⁺; ¹H NMR (400 MHz, METHANOL-d4) δ = 8.21 (br s, |
| | 2H), 8.00-7.77 (m, 2H), 7.02 (br s, 1H), 6.49 (br s, 1H), 4.72-4.49 (m, 1H), |
| | 4.17-3.80 (m, 3H), 3.56-3.38 (m, 4H), 3.16-2.90 (m, 2H), 2.17 (br s, 2H), |
| | 2.13-1.73 (m, 6H); NH wasn't observed |
| I-517 | MS m/z 500.3 [M + H]⁺; ¹H NMR (400 MHz, METHANOL-d4) δ = 8.27-8.06 |
| | (m, 2H), 7.98-7.77 (m, 2H), 7.01 (br s, 1H), 6.47 (br s, 1H), 4.54 (br s, 1H), |
| | 4.09-3.84 (m, 3H), 3.82-3.71 (m, 1H), 3.68-3.47 (m, 2H), 3.27-3.05 (m, |
| | 1H), 2.85-2.64 (m, 2H), 2.37-2.17 (m, 2H), 2.17-1.99 (m, 2H), 1.98-1.71 |
| | (m, 2H); NH wasn't observed |
| I-519 | MS m/z 458.3 [M + H]⁺; ¹H NMR (400 MHz, Methanol-d4) δ 7.89-7.79 (m, |
| | 1H), 7.72-7.64 (m, 1H), 7.40-7.33 (m, 1H), 7.33-7.28 (m, 1H), 7.05-6.64 |
| | (m, 2H), 6.60-6.52 (m, 1H), 4.58-4.38 (m, 1H), 3.24-3.08 (m, 1H), 2.85- |
| | 2.67 (m, 1H), 2.48-2.19 (m, 5H), 2.17-2.00 (m, 1H), 1.96-1.84 (m, 1H), |
| | 1.83-1.70 (m, 1H), 1.69-1.53 (m, 1H). NH peak not observed |
| I-520 | MS m/z 488.3 [M + H]⁺; ¹H NMR (400 MHz, Methanol-d4) δ 7.92-7.82 (m, |
| | 1H), 7.73-7.64 (m, 1H), 7.42-7.33 (m, 1H), 7.33-7.26 (m, 1H), 7.06-6.64 |
| | (m, 2H), 6.60-6.51 (m, 1H), 4.56-4.40 (m, 1H), 3.80-3.64 (m, 2H), 3.24- |
| | 3.08 (m, 1H), 2.82-2.53 (m, 3H), 2.53-2.35 (m, 2H), 2.16-1.97 (m, 1H), |
| | 1.95-1.80 (m, 1H), 1.80-1.59 (m, 2H). NH peak not observed |
| I-522 | MS m/z 445.5 [M + H]⁺; ¹H NMR (400 MHz, Methanol-d4) δ 7.87-7.80 (m, |
| | 1H), 7.73-7.64 (m, 1H), 7.40-7.33 (m, 1H), 7.32-7.27 (m, 1H), 7.07-6.65 |
| | (m, 2H), 6.61-6.50 (m, 1H), 4.32-4.20 (m, 1H), 2.72-2.60 (m, 2H), 2.33- |
| | 2.18 (m, 2H), 1.44 (s, 3H). NH and OH peak not observed |
| I-523 | MS m/z 395.6 [M + H]⁺; ¹H NMR (400 MHz, Methanol-d4) δ 8.13-8.00 (m, |
| | 1H), 7.96-7.85 (m, 1H), 7.34-7.18 (m, 1H), 7.13-7.01 (m, 1H), 6.96-6.81 |
| | (m, 2H), 4.31-4.13 (m, 1H), 2.72-2.58 (m, 2H), 2.35-2.16 (m, 2H), 1.45 (s, |
| | 3H). NH and OH peak not observed |
| I-524 | MS m/z 461.5 [M + H]⁺; ¹H NMR (400 MHz, Methanol-d4) δ 7.80-7.74 (m, |
| | 1H), 7.73-7.66 (m, 2H), 7.62-7.55 (m, 1H), 7.16-6.70 (m, 2H), 4.30-4.16 |
| | (m, 1H), 3.79-3.64 (m, 1H), 2.50-2.36 (m, 1H), 2.19-2.06 (m, 1H), 2.05- |
| | 1.96 (m, 1H), 1.95-1.83 (m, 1H), 1.58-1.17 (m, 4H). |
| I-525 | MS m/z 447.5 [M + H]⁺; ¹H NMR (400 MHz, Methanol-d4) δ 7.81-7.73 (m, |
| | 1H), 7.73-7.67 (m, 2H), 7.60-7.56 (m, 1H), 7.13-6.71 (m, 2H), 4.33-4.19 |
| | (m, 1H), 2.72-2.59 (m, 2H), 2.31-2.18 (m, 2H), 1.44 (s, 3H). |
| I-526 | MS m/z 411.5 [M + H]⁺; ¹H NMR (400 MHz, Methanol-d4) δ 8.06 (s, 1H), 7.69 |
| | (d, J = 7.7 Hz, 1H), 7.36-7.29 (m, 1H), 7.27 (s, 1H), 7.05 (s, 1H), 4.21-4.06 |
| | (m, 1H), 3.78-3.65 (m, 1H), 2.49-2.35 (m, 1H), 2.20-2.09 (m, 1H), 2.05- |
| | 1.95 (m, 1H), 1.95-1.83 (m, 1H), 1.59-1.19 (m, 4H). |
| I-530 | MS m/z: 472.2 [M + H]⁺; ¹H NMR (400 MHz, MeOD) δ: 8.16 (s, 1H), 8.10 (d, |
| | J = 8.4 Hz, 1H), 7.86 (d, J = 2.4 Hz, 1H), 7.83 (d, J = 8.0 Hz, 1H), 6.97-6.95 |
| | (m, 1H), 6.41 (d, J = 3.6 Hz, 1H), 4.48-4.46 (m, 1H), 3.30-3.22 (m, 1H), |
| | 2.85-2.79 (m, 2H), 2.43-2.38 (m, 2H), 2.14-2.10 (m, 1H), 1.87-1.85 (m, |
| | 1H), 1.76-1.75 (m, 1H), 1.62-1.60 (m, 1H), 1.13 (d, J = 6.4 Hz, 6H). NH and |
| | OH not observed. |
| I-531 | MS m/z: 419.2 [M + H]⁺; ¹H NMR (400 MHz, Methanol-d4) δ: 8.17 (s, 1H), |
| | 8.11 (d, J = 8.0 Hz, 1H), 7.90-7.79 (m, 2H), 7.00 (t, J = 8.0 Hz, 1H), 6.44 (d, |
| | J = 3.6 Hz, 1H), 3.73 (s, 2H), 1.32 (s, 6H). NH and OH not observed. |
| I-532 | MS m/z: 431.1 [M + H]⁺; ¹H NMR (400 MHz, MeOD) δ 8.16 (s, 1H), 8.10 (d, |
| | J = 8.4 Hz, 1H), 7.86 (d, J = 2.4 Hz, 1H), 7.83 (d, J = 8.0 Hz, 1H), 6.97-6.95 |
| | (m, 1H), 6.41 (d, J = 2.8 Hz, 1H), 4.36-4.26 (m, 1H), 2.68-2.63 (m, 2H), |
| | 2.30-2.24 (m, 2H), 1.44 (s, 3H). NH and OH not observed. |
| I-533 | MS m/z: 390.2 [M + H]⁺; ¹H NMR (400 MHz, DMSO) δ 7.97 (d, J = 1.8 Hz, |
| | 1H), 7.72 (s, 1H), 7.59 (d, J = 8.1 Hz, 1H), 7.48 (d, J = 7.8 Hz, 1H), 7.22 (d, J = |
| | 6.0 Hz, 1H), 6.95-6.78 (m, 1H), 6.26 (d, J = 3.0 Hz, 1H), 4.30 (s, 1H), 3.06 (d, |
| | J = 7.2 Hz, 1H), 2.71 (d, J = 10.6 Hz, 1H), 2.22 (s, 3H), 2.02 (d, J = 8.7 Hz, |
| | 1H), 1.96-1.85 (m, 2H), 1.80-1.72 (m, 1H), 1.66-1.55 (m, 1H), 1.48-1.36 |
| | (m, 1H). |
| I-534 | MS m/z: 416.3 [M + H]⁺; ¹H NMR (400 MHz, DMSO-d6) d: 7.96 (d, J = 1.6 |
| | Hz, 1H), 7.65-7.54 (m, 1H), 7.49-7.36 (m, 2H), 7.20 (d, J = 7.8 Hz, 1H), |
| | 6.93-6.83 (m, 1H), 6.27 (d, J = 3.0 Hz, 1H), 4.37-4.19 (m, 1H), 3.06 (d, J = |
| | 9.8 Hz, 1H), 2.77-2.61 (m, 1H), 2.22 (s, 3H), 2.16-2.10 (m, 1H), 2.06-1.96 |
| | (m, 1H), 1.96-1.83 (m, 2H), 1.80-1.69 (m, 1H), 1.66-1.53 (m, 1H), 1.47- |
| | 1.34 (m, 1H), 1.12-1.01 (m, 2H), 0.88-0.77 (m, 2H). |
| I-536 | MS m/z: 417.2 [M + H]⁺; ¹H NMR (400 MHz, DMSO) δ 8.04 (d, J = 1.8 Hz, |
| | 1H), 7.88-7.75 (m, 2H), 7.70 (s, 1H), 7.60-7.50 (m, 1H), 7.36 (t, J = 70.2 |
| | Hz, 1H), 7.00-6.93 (m, 1H), 6.53-6.43 (m, 1H), 4.90 (s, 1H), 3.64 (d, J = 5.9 |
| | Hz, 2H), 1.21 (s, 6H). NH and OH not observed. |
| I-537 | MS m/z: 429.1 [M + H]⁺; ¹H NMR (400 MHz, DMSO-d6) δ 7.97 (d, J = 1.8 Hz, |
| | 1H), 7.85-7.74 (m, 3H), 7.69 (s, 1H), 7.36 (t, J = 73.4 Hz, 1H), 7.00-6.92 |
| | (m, 1H), 6.48 (d, J = 3.0 Hz, 1H), 5.07 (s, 1H), 4.24-4.20 (m, 1H), 2.48-2.41 |
| | (m, 2H), 2.26-2.15 (m, 2H), 1.34 (s, 3H). |
| I-539 | MS m/z: 331.1 [M + H]⁺; ¹H NMR (400 MHz, Methanol-d4) δ 7.76 (d, J = 1.6 |
| | Hz, 1H), 6.95 (t, J = 3.6 Hz, 1H), 6.62 (s, 1H), 6.58-6.53 (m, 2H), 3.71 (s, |
| | 2H), 3.34 (s, 3H), 1.31 (s, 6H). NH and OH not observed. |
| I-540 | MS m/z: 357.2 [M + H]⁺; ¹H NMR (400 MHz, CDCl₃) δ 7.56 (s, 1H), 6.96 (s, |
| | 1H), 6.93 (d, J = 5.6 Hz, 1H), 6.60 (s, 1H), 6.44 (d, J = 12.0 Hz, 1H), 3.74 (s, |
| | 2H), 1.89-1.83 (m, 1H), 1.37 (s, 6H), 1.04-0.99 (m, 2H), 0.78-0.74 (m, |
| | 2H). |
| I-541 | MS m/z: 410.2 [M + H]⁺; ¹H NMR (400 MHz, DMSO-d6) δ 7.90 (d, J = 2.0 Hz, |
| | 1H), 7.16 (d, J = 8.0 Hz, 1H), 6.90 (t, J = 3.2 Hz, 1H), 6.54 (s, 1H), 6.50 (d, J = |
| | 10.4 Hz, 1H), 6.32 (d, J = 4.0 Hz, 1H), 4.26-4.24 (m, 1H), 3.11 (d, J = 7.6 Hz, |
| | 1H), 2.80-2.74 (m, 2H), 2.13 (t, J = 10.4 Hz, 2H), 2.06-2.03 (m, 1H), 1.94- |
| | 1.90 (m, 1H), 1.77-1.74 (m, 1H), 1.56-1.40 (m, 2H), 0.99 (d, J = 6.4 Hz, |
| | 6H), 1.00-0.92 (m, 2H), 0.73-0.69 (m, 2H). |
| I-542 | MS m/z: 450.2 [M + H]⁺; ¹H NMR (400 MHz, DMSO-d6) d: 7.88 (d, J = 1.2 |
| | Hz, 1H), 7.21 (d, J = 7.6 Hz, 1H), 6.89 (t, J = 3.2 Hz, 1H), 6.54 (s, 1H), 6.47 (d, |
| | J = 11.2 Hz, 1H), 6.33 (d, J = 3.6 Hz, 1H), 4.31-4.23 (m, 1H), 3.29-3.10 (m, |
| | 3H), 2.94-2.88 (m, 1H), 2.37 (t, J = 10.4 Hz, 2H), 2.06-2.03 (m, 1H), 1.95- |
| | 1.87 (m, 1H), 1.78-1.75 (m, 1H), 1.65-1.54 (m, 1H), 1.50-1.40 (m, 1H), |
| | 1.01-0.93 (m, 2H), 0.73-0.67 (m, 2H). |
| I-552 | MS m/z 446.4, 448.4 [M + H]⁺; ¹H NMR (400 MHz, DMSO) δ 8.17 (s, 1H, |
| | formic acid proton), 8.11 (s, 1H), 7.90 (d, J = 8.5 Hz, 1H), 7.51 (s, 1H), 7.19 (d, |
| | J = 9.7 Hz, 2H), 7.13 (d, J = 8.3 Hz, 1H), 7.08 (s, 1H), 4.26-4.30 (m, 1H), 3.42- |
| | 3.48 (m, 2H), 3.17-3.02 (m, 2H), 2.70-2.78 (m, 2H), 2.47-2.20 (m, 2H), |
| | 1.96-2.02 (m, 1H), 1.72-1.80 (m, 1H), 1.44-1.60 (m, 2H), 0.95 (d, J = 6.3 Hz, |
| | 3H). NH peak not observed |
| I-562 | MS m/z 359.5 [M + H]⁺; ¹H NMR (400 MHz, METHANOL-d4) δ 7.83 (s, 1H), |
| | 6.96 (m, 1H), 6.63 (m, 1H), 6.37 (m, 2H), 4.24 (t, J = 7.8 Hz, 1H), 3.82 (s, |
| | 3H), 2.74-2.54 (m, 2H), 2.26 (t, J = 9.8 Hz, 2H), 1.44 (m, 3H). NH and OH |
| | peak not observed |
| I-563 | MS m/z 370.1 [M + H]⁺; ¹H NMR (400 MHz, METHANOL-d4) δ 8.43 (s, 1H, |
| | formic acid), 7.81 (s, 1H), 6.97 (s, 1H), 6.66 (s, 1H), 6.62 (d, J = 10.4 Hz, 1H), |
| | 6.58 (s, 1H), 4.61-4.52 (m, 1H), 3.85-3.71 (m, 1H), 3.38-3.33 (m, 1H), |
| | 3.10-2.90 (m, 2H), 2.84 (s, 3H), 2.66 (q, J = 7.3 Hz, 2H), 2.28-2.19 (m, 1H), |
| | 2.18-2.10 (m, 1H), 2.01-1.80 (m, 2H), 1.27 (t, J = 7.6 Hz, 3H). 2H not |
| | observed (NH and OH). |
| I-565 | MS m/z 472.3 [M + H]⁺; ¹H NMR (400 MHz, Methanol-d4) δ 7.88-7.80 (m, |
| | 1H), 7.70-7.65 (m, 1H), 7.39-7.29 (m, 2H), 7.06-6.64 (m, 2H), 6.59-6.52 |
| | (m, 1H), 4.58-4.41 (m, 1H), 3.31-3.19 (m, 1H), 2.94-2.77 (m, 1H), 2.69- |
| | 2.50 (m, 2H), 2.41-2.22 (m, 2H), 2.21-2.05 (m, 1H), 1.98-1.83 (m, 1H), |
| | 1.84-1.55 (m, 2H), 1.22-1.10 (m, 3H). NH peak not observed. |
| I-566 | MS m/z 474.3 [M + H]⁺; ¹H NMR (400 MHz, Methanol-d4) δ 7.81-7.74 (m, |
| | 1H), 7.73-7.67 (m, 2H), 7.61-7.55 (m, 1H), 7.13-6.72 (m, 2H), 4.55-4.40 |
| | (m, 1H), 3.28-3.16 (m, 1H), 2.89-2.74 (m, 1H), 2.63-2.44 (m, 2H), 2.37- |
| | 2.18 (m, 2H), 2.16-2.03 (m, 1H), 1.94-1.83 (m, 1H), 1.82-1.55 (m, 2H), |
| | 1.21-1.10 (m, 3H). NH peak not observed. |
| I-568 | MS m/z 431.2 [M + H]⁺; ¹H NMR (400 MHz, DMSO-d6)δ 8.08 (d, J = 1.8 Hz, |
| | 1H), 7.83 (d, J = 8.0 Hz, 1H), 7.77 (d, J = 8.4 Hz, 1H), 7.69 (s, 1H), 7.43 (t, J = |
| | 6.0 Hz, 1H), 7.27 (t, J = 73.4 Hz, 1H), 7.00-6.93 (m, 1H), 6.49 (d, J = 3.1 Hz, |
| | 1H), 3.74 (d, J = 5.9 Hz, 2H), 3.20 (s, 3H), 1.22 (s, 6H). |
| I-569 | MS m/z 384.3 [M + H]⁺; ¹H NMR (400 MHz, DMSO-d6) δ: 9.93 (s, 1H), 7.90 |
| | (d, J = 1.7 Hz, 1H), 7.15 (d, J = 7.8 Hz, 1H), 6.95-6.81 (m, 1H), 6.62 (s, 1H), |
| | 6.60 (d, J = 10.8 Hz, 1H), 6.31 (d, J = 3.8 Hz, 1H), 4.24 (br s, 1H), 3.11 (d, J = |
| | 7.0 Hz, 1H), 2.83-2.71 (m, 2H), 2.30 (s, 3H), 2.13 (t, J = 10.1 Hz, 2H), 2.07- |
| | 1.97 (m, 1H), 1.81-1.70 (m, 1H), 1.62-1.49 (m, 1H), 1.47-1.30 (m, 1H), |
| | 0.99 (d, J = 6.6 Hz, 6H). |
| I-570 | MS m/z 424.2 [M + H]⁺; ¹H NMR (400 MHz, DMSO-d6) d: 9.92 (s, 1H), 7.88 |
| | (d, J = 1.8 Hz, 1H), 7.20 (d, J = 7.8 Hz, 1H), 6.92-6.85 (m, 1H), 6.62 (s, 1H), |
| | 6.60 (d, J = 10.6 Hz, 1H), 6.31 (d, J = 3.8 Hz, 1H), 4.29 (br s, 1H), 3.31-3.20 |
| | (m, 3H), 2.93 (d, J = 10.8 Hz, 1H), 2.37 (t, J = 10.4 Hz, 2H), 2.30 (s, 3H), 2.04 |
| | (d, J = 9.2 Hz, 1H), 1.81-1.70 (m, 1H), 1.67-1.54 (m, 1H), 1.52-1.38 (m, |
| | 1H). |
| I-573 | MS m/z 427.2 [M + H]⁺; ¹H NMR (400 MHz, DMSO-d6) d: 7.94 (d, J = 1.6 Hz, |
| | 1H), 7.70 (d, J = 6.0 Hz, 1H), 7.61 (d, J = 8.4 Hz, 1H), 7.40 (t, J = 73.2 Hz, 1H), |
| | 7.25 (t, J = 73.6 Hz, 1H), 7.22 (d, J = 3.2 Hz, 1H), 7.22 (d, J = 2 Hz, 1H), 6.93 |
| | (t, J = 3.6 Hz, 1H), 6.42 (t, J = 2.8 Hz, 1H), 5.02 (s, 1H), 4.24-4.18 (m, 1H), |
| | 2.51-2.49 (m, 2H), 2.22-2.17 (m, 2H), 1.33 (s, 3H). |
| I-575 | MS m/z 396.1 [M + H]⁺; ¹H NMR (400 MHz, CD₃OD) δ 8.48 (s, 1H), 8.04 (d, J = |
| | 8.0 Hz, 1H), 7.86 (d, J = 2.0 Hz, 1H), 7.38-7.27 (m, 2H), 7.16 (dt, J = 6.8, |
| | 3.6 Hz, 2H), 5.62-5.33 (m, 1H), 4.33-4.22 (m, 1H), 4.14 (dd, J = 15.0, 2.8 |
| | Hz, 1H), 3.99 (dd, J = 15.0, 7.0 Hz, 1H), 3.72-3.51 (m, 2H), 2.63-2.51 (m, |
| | 1H), 2.35-2.11 (m, 1H). NH peak not observed. |
| I-576 | MS m/z 414.3 [M + H]⁺; ¹H NMR (400 MHz, DMSO-d6) d: 9.93 (s, 1H), 7.87 |
| | (d, J = 1.8 Hz, 1H), 7.23 (d, J = 7.7 Hz, 1H), 6.93-6.86 (m, 1H), 6.54 (s, 1H), |
| | 6.48 (d, J = 11.0 Hz, 1H), 6.34 (d, J = 3.7 Hz, 1H), 5.01 (d, J = 47.6 Hz, 1H), |
| | 4.59 (br s, 1H), 3.17 (d, J = 9.8 Hz, 1H), 3.03 (t, J = 10.8 Hz, 1H), 2.47-2.39 |
| | (m, 2H), 2.35-2.27 (m, 1H), 2.27-2.12 (m, 1H), 2.05 (t, J = 10.1 Hz, 1H), |
| | 1.98-1.87 (m, 1H), 1.89-1.67 (m, 1H), 1.02 (t, J = 7.2 Hz, 3H), 1.00-0.96 |
| | (m, 2H), 0.77-0.67 (m, 2H). |
| I-577 | MS m/z 337.1 [M + H]⁺; ¹H NMR (400 MHz, CD₃OD) δ 7.65 (d, J = 2.0 Hz, |
| | 1H), 6.85 (t, J = 3.2 Hz, 1H), 6.69-6.67 (m, 2H), 6.43 (d, J = 3.6 Hz, 1H), |
| | 4.07-4.09 (m, 1H), 3.59-3.64 (m, 1H), 3.41-3.47 (m, 1H), 1.17 (d, J = 6.4 Hz, |
| | 3H). NH peak not observed. |
| I-582 | MS m/z 370.4 [M + H]⁺; ¹H NMR (400 MHz, METHANOL-d4) δ 8.53 (s, 1H, |
| | formic acid), 7.76 (s, 1H), 7.02 (s, 1H), 6.68-6.55 (m, 3H), 4.13 (d, J = 14.9 |
| | Hz, 1H), 3.92-3.78 (m, 2H), 3.72-3.48 (m, 2H), 3.26-3.10 (m, 2H), 2.36 (s, |
| | 3H), 2.33-2.25 (m, 1H), 2.19-1.92 (m, 3H), 1.44 (t, J = 7.2 Hz, 3H). 2H not |
| | observed (NH and OH). |
| I-583 | MS m/z 396.4 [M + H]⁺; ¹H NMR (400 MHz, METHANOL-d4) δ 8.45 (s, 1 H, |
| | formic acid), 7.76 (s, 1H), 7.02 (s, 1H), 6.62 (s, 1H), 6.54 (s, 1H), 6.48 (d, J = |
| | 10.6 Hz, 1H), 4.15 (d, J = 14.1 Hz, 1H), 3.92-3.81 (m, 2H), 3.74-3.57 (m, |
| | 2H), 3.28-3.17 (m, 2H), 2.37-2.24 (m, 1H), 2.19-1.86 (m, 4H), 1.44 (t, J = |
| | 7.1 Hz, 3H), 1.04 (d, J = 7.0 Hz, 2H), 0.80-0.69 (m, 2H). 2H not observed |
| | (NH and OH). NH peak not observed. |
| I-586 | MS m/z 325.3 [M + H]⁺; ¹H NMR (400 MHz, METHANOL-d4) δ 7.87 (d, J = |
| | 8.5 Hz, 1H), 7.83 (s, 1H), 7.23 (d, J = 2.6 Hz, 1H), 7.04 (s, 1H), 6.72 (d, J = 7.5 |
| | Hz, 1H), 6.69 (s, 1H), 4.23-4.12 (m, 1H), 3.73-3.64 (m, 1H), 3.55-3.44 (m, |
| | 1H), 1.98-1.86 (m, 1H), 1.28 (d, J = 6.3 Hz, 3H), 1.05-0.95 (m, 2H), 0.81- |
| | 0.71 (m, 2H). 3H not observed (NH and 2 OH). |
| I-587 | MS m/z 315.4 [M + H]⁺; ¹H NMR (400 MHz, METHANOL-d4) δ 7.94 (d, J = |
| | 8.8 Hz, 1H), 7.83 (s, 1H), 7.26 (s, 1H), 7.04 (s, 1H), 6.58 (d, J = 10.0 Hz, 1H), |
| | 6.53 (s, 1H), 4.22-4.11 (m, 1H), 3.83 (s, 3H), 3.71-3.63 (m, 1H), 3.53-3.44 |
| | (m, 1H), 1.27 (d, J = 6.1 Hz, 3H). 3H not observed (NH and 2 OH). |
| I-588 | MS m/z 353.3 [M + H]⁺; ¹H NMR (400 MHz, METHANOL-d4) δ 8.15 (d, J = |
| | 8.4 Hz, 1H), 7.88 (s, 1H), 7.30-7.22 (m, 3H), 7.09 (s, 1H), 4.23-4.13 (m, |
| | 1H), 3.78-3.66 (m, 1H), 3.58-3.46 (m, 1H), 1.28 (d, J = 6.1 Hz, 3H). 3H not |
| | observed (NH and 2 OH). |
| I-589 | MS m/z 431.3 [M + H]⁺; ¹H NMR (400 MHz, METHANOL-d4) δ 8.17 (s, 1H), |
| | 8.11 (d, J = 7.6 Hz, 1H), 7.84 (d, J = 7.9 Hz, 1H), 7.80 (s, 1H), 6.98 (bs, 1H), |
| | 6.43 (s, 1H), 3.99-3.86 (m, 2H), 3.83-3.75 (m, 1H), 3.75-3.59 (m, 3H), |
| | 2.94-2.81 (m, 1H), 2.22-2.09 (m, 1H), 1.87-1.74 (m, 1H). 1H not observed |
| | (NH). |
| I-590 | MS m/z 431.3 [M + H]⁺; ¹H NMR (400 MHz, METHANOL-d4) δ 8.17 (s, 1H), |
| | 8.11 (d, J = 7.9 Hz, 1H), 7.84 (d, J = 7.8 Hz, 1H), 7.80 (s, 1H), 6.98 (s, 1H), |
| | 6.43 (s, 1H), 4.00-3.85 (m, 2H), 3.85-3.74 (m, 1H), 3.74-3.60 (m, 3H), |
| | 2.94-2.80 (m, 1H), 2.23-2.08 (m, 1H), 1.88-1.72 (m, 1H). 1H not observed |
| | (NH). |
| I-591 | MS m/z 456.4 [M + H]⁺; ¹H NMR (400 MHz, METHANOL-d4) δ 7.84-7.69 |
| | (m, 2H), 7.63 (m, 2H), 7.13-6.67 (m, 1H), 6.39 (s, 1H), 4.54-4.41 (m, 1H), |
| | 2.93-2.82 (m, 1H), 2.47 (m, 5H), 2.33 (s, 3H), 2.19 (s, 1H), 2.17-2.06 (m, |
| | 1H), 1.99-1.88 (m, 1H), 1.85-1.73 (m, 1H), 1.73-1.58 (m, 1H). NH peak |
| | was not observed |
| I-592 | MS m/z 488.3 [M + H]⁺; ¹H NMR (400 MHz, Methanol-d4) δ 7.81-7.66 (m, |
| | 3H), 7.62-7.56 (m, 1H), 7.14-6.72 (m, 2H), 4.57-4.40 (m, 1H), 3.48-3.35 |
| | (m, 1H), 3.11-2.92 (m, 2H), 2.67-2.44 (m, 2H), 2.27-2.08 (m, 1H), 2.04- |
| | 1.89 (m, 1H), 1.87-1.57 (m, 2H), 1.20 (s, 6H). NH peak not observed |
| I-593 | MS m/z 397.3 [M + H]⁺; ¹H NMR (400 MHz, Methanol-d4) δ 7.88-7.53 (m, |
| | 2H), 7.34-7.07 (m, 2H), 6.93-6.73 (m, 1H), 4.35-4.08 (m, 1H), 2.77-2.53 |
| | (m, 2H), 2.39-2.13 (m, 2H), 1.43 (s, 3H). NH and OH peak not observed |
| I-594 | MS m/z 424.4 [M + H]⁺; ¹H NMR (400 MHz, Methanol-d4) δ 8.52 (s, 1H, |
| | formic acid proton), 7.77-7.69 (m, 1H), 7.68-7.59 (m, 1H), 7.29-7.14 (m, |
| | 2H), 6.91-6.76 (m, 1H), 4.58-4.42 (m, 1H), 3.67-3.51 (m, 1H), 3.24-3.12 |
| | (m, 1H), 2.99-2.83 (m, 2H), 2.77-2.52 (m, 2H), 2.27-2.13 (m, 1H), 2.11- |
| | 1.97 (m, 1H), 1.95-1.64 (m, 2H), 1.38-1.19 (m, 3H). NH and OH peak not |
| | observed |
| I-595 | MS m/z 371.3 [M + H]⁺. ¹H NMR (400 MHz, Methanol-d4) δ 7.81-7.55 (m, |
| | 2H), 7.30-7.13 (m, 2H), 6.94-6.75 (m, 1H), 4.27-4.07 (m, 1H), 3.79-3.61 |
| | (m, 1H), 3.58-3.45 (m, 1H), 1.35-1.21 (m, 3H). NH and OH peak not |
| | observed |
| I-599 | MS m/z 382.4 [M + H]⁺; ¹H NMR (400 MHz, Methanol-d4) δ 7.77-7.65 (m, |
| | 1H), 7.48-7.38 (m, 1H), 6.85-6.77 (m, 1H), 6.72-6.62 (m, 2H), 4.47-4.32 |
| | (m, 1H), 3.23-3.10 (m, 1H), 2.90-2.69 (m, 1H), 2.51-2.27 (m, 5H), 2.18- |
| | 2.00 (m, 1H), 1.98-1.84 (m, 2H), 1.83-1.56 (m, 2H), 1.06-0.94 (m, 2H), |
| | 0.81-0.68 (m, 2H). NH and OH peak not observed |
| I-600 | MS m/z 396.4 [M + H]⁺; ¹H NMR (400 MHz, Methanol-d4) δ 7.75-7.65 (m, |
| | 1H), 7.47-7.38 (m, 1H), 6.86-6.77 (m, 1H), 6.72-6.61 (m, 2H), 4.52-4.33 |
| | (m, 1H), 3.43-3.31 (m, 1H), 3.04-2.85 (m, 1H), 2.79-2.60 (m, 2H), 2.53- |
| | 2.33 (m, 2H), 2.22-2.06 (m, 1H), 2.00-1.87 (m, 2H), 1.84-1.59 (m, 2H), |
| | 1.25-1.15 (m, 3H), 1.06-0.95 (m, 2H), 0.79-0.67 (m, 2H). NH and OH |
| | peak not observed |
| I-601 | MS m/z 400.4 [M + H]⁺; ¹H NMR (400 MHz, Methanol-d4) δ 7.70-7.58 (m, |
| | 1H), 6.78-6.68 (m, 1H), 6.51-6.46 (m, 1H), 6.45-6.38 (m, 1H), 4.54-4.35 |
| | (m, 1H), 3.31-3.17 (m, 1H), 2.94-2.75 (m, 1H), 2.57-2.30 (m, 5H), 2.18- |
| | 2.03 (m, 1H), 2.00-1.86 (m, 2H), 1.84-1.56 (m, 2H), 1.08-0.95 (m, 2H), |
| | 0.79-0.67 (m, 2H). NH and OH peak not observed |
| I-602 | MS m/z 414.4 [M + H]⁺; ¹H NMR (400 MHz, Methanol-d4) δ 7.71-7.57 (m, |
| | 1H), 6.79-6.69 (m, 1H), 6.52-6.46 (m, 1H), 6.45-6.38 (m, 1H), 4.53-4.39 |
| | (m, 1H), 3.48-3.35 (m, 1H), 3.05-2.91 (m, 1H), 2.81-2.63 (m, 2H), 2.56- |
| | 2.33 (m, 2H), 2.21-2.06 (m, 1H), 2.02-1.59 (m, 4H), 1.26-1.16 (m, 3H), |
| | 1.09-0.95 (m, 2H), 0.79-0.67 (m, 2H). NH and OH peak not observed |
| I-604 | MS m/z 440.3 [M + H]⁺; ¹H NMR (400 MHz, Methanol-d4) δ 7.78-7.66 (m, |
| | 1H), 7.63-7.51 (m, 1H), 6.92-6.75 (m, 3H), 4.56-4.38 (m, 1H), 3.61-3.44 |
| | (m, 1H), 3.19-3.02 (m, 1H), 2.93-2.74 (m, 2H), 2.71-2.48 (m, 2H), 2.25- |
| | 2.11 (m, 1H), 2.08-1.94 (m, 1H), 1.92-1.65 (m, 2H), 1.33-1.16 (m, 3H). |
| | NH and OH peak not observed |
| I-606 | MS m/z 420.2 [M + H]⁺; ¹H NMR (400 MHz, METHANOL-d4) δ 8.01-7.77 |
| | (m, 1H), 7.25-6.87 (m, 3H), 6.49 (br s, 1H), 4.74-4.53 (m, 1H), 4.06-3.80 |
| | (m, 1H), 3.61-3.41 (m, 1H), 3.30-3.19 (m, 2H), 3.15-2.97 (m, 2H), 2.35- |
| | 2.07 (m, 5H), 2.07-1.85 (m, 2H), 1.39 (br s, 3H); 2H (NH and OH) wasn't |
| | observed |
| I-607 | MS m/z 424.3 [M + H]⁺; ¹H NMR (400 MHz, DMSO-d6) δ 8.25 (br s, 1H), 7.82- |
| | 7.66 (m, 1H), 7.19 (d, J = 9.1 Hz, 1H), 7.14-7.11 (m, 1H), 7.13-7.07 (m, |
| | 1H), 6.20 (s, 1H), 4.27 (m, 1H), 3.05 (m, 1H), 2.75-2.64 (m, 1H), 2.22 (m, |
| | 6H), 2.03-1.84 (m, 3H), 1.76 (m, 1H), 1.67-1.51 (m, 1H), 1.49-1.34 (m, |
| | 1H). OH peak not observed. |
| I-608 | MS m/z 363.1, 365.1 [M + H]⁺; ¹H NMR (400 MHz, MeOD) δ 7.35 (d, J = 2.4 |
| | Hz, 1H), 7.00 (t, J = 3.6 Hz, 1H), 6.90-6.92 (m, 2H), 6.77 (dd, J = 2.0 Hz, 10.8 |
| | Hz, 1H), 5.31-5.34 (m, 1H), 4.49-4.51 (m, 1H), 3.89-3.94 (m, 2H), 3.67 (dd, |
| | J = 3.2 Hz, 12 Hz, 1H), 2.14-2.22 (m, 1H), 1.98-2.03 (m, 1H), 1.62-1.65 |
| | (m, 1H). NH and OH not observed |
| I-609 | MS m/z 422.2 [M + H]⁺; ¹H NMR (400 MHz, DMSO-d6) δ 8.12-8.05 (m, 2H), |
| | 7.48 (d, J = 7.6 Hz, 1H), 7.24 (d, J = 3.7 Hz, 1H), 7.12-7.07 (m, 1H), 6.95- |
| | 6.88 (m, 2H), 4.27 (br s, 1H), 3.16 (d, J = 6.9 Hz, 1H), 2.83 (d, J = 10.7 Hz, |
| | 1H), 2.39 (q, J = 7.2 Hz, 2H), 2.10-2.02 (m, 1H), 1.99-1.86 (m, 2H), 1.84- |
| | 1.73 (m, 1H), 1.70-1.53 (m, 1H), 1.52-1.43 (m, 1H), 1.02 (t, J = 7.1 Hz, 3H). |
| | one proton is overlapped with d-solvent peak |
| I-610 | MS m/z: 429.1 [M + H]⁺; ¹H NMR (400 MHz, DMSO) δ 7.96 (d, J = 1.9 Hz, |
| | 1H), 7.88 (t, J = 5.5 Hz, 1H), 7.84-7.76 (m, 2H), 7.70 (s, 1H), 7.36 (t, J = 73.4 |
| | Hz, 1H), 7.00-6.93 (m, 1H), 6.49 (d, J = 3.8 Hz, 1H), 4.28-4.19 (m, 1H), |
| | 3.84 (dd, J = 14.2, 7.2 Hz, 1H), 3.74-3.54 (m, 3H), 2.01 (td, J = 12.4, 7.3 Hz, |
| | 1H), 1.95-1.81 (m, 2H), 1.73-1.60 (m, 1H). |
| I-611 | MS m/z: 429.1 [M + H]⁺; ¹H NMR (400 MHz, DMSO) δ 7.96 (dd, J = 2.8, 1.1 |
| | Hz, 1H), 7.88 (t, J = 5.5 Hz, 1H), 7.83-7.76 (m, 2H), 7.70 (s, 1H), 7.36 (t, J = |
| | 73.4 Hz, 1H), 6.96 (dd, J = 3.8, 2.9 Hz, 1H), 6.49 (dd, J = 3.9, 1.1 Hz, 1H), |
| | 4.36-4.14 (m, 1H), 3.89-3.78 (m, 1H), 3.75-3.56 (m, 3H), 2.07-1.95 (m, 1H), |
| | 1.92-1.81 (m, 2H), 1.71-1.62 (m, 1H). |
| I-612 | MS m/z 440.2 [M + H]⁺; ¹H NMR (400 MHz, DMSO-d6) δ 7.96 (d, J = 2.1 Hz, |
| | 1H), 7.62 (d, J = 8.3 Hz, 1H), 7.42 (t, J = 73.2 Hz, 1H), 7.25-7.16 (m, 4H), |
| | 6.96-6.91 (m, 1H), 6.43 (d, J = 3.8 Hz, 1H), 4.43-4.21 (m, 1H), 3.12-3.06 |
| | (m, 1H), 2.76-2.63 (m, 1H), 2.21 (s, 3H), 2.07-1.99 (m, 1H), 1.97-1.86 (m, |
| | 2H), 1.82-1.72 (m, 1H), 1.68-1.56 (m, 1H), 1.47-1.35 (m, 1H). |
| I-613 | MS m/z 454.2 [M + H]⁺; ¹H NMR (400 MHz, DMSO-d6) δ 7.95 (d, J = 1.9 Hz, |
| | 1H), 7.62 (d, J = 8.3 Hz, 1H), 7.42 (t, J = 15.2 Hz, 1H), 7.32-7.12 (m, 4H), |
| | 6.97-6.88 (m, 1H), 6.43 (d, J = 3.2 Hz, 1H), 4.37-4.21 (m, 1H), 3.17 (d, J = |
| | 7.0 Hz, 1H), 2.83 (d, J = 10.3 Hz, 1H), 2.39 (q, J = 7.1 Hz, 2H), 2.08-2.01 (m, |
| | 1H), 1.96-1.86 (m, 2H), 1.82-1.72 (m, 1H), 1.69-1.54 (m, 1H), 1.50-1.40 |
| | (m, 1H), 1.02 (t, J = 7.1 Hz, 3H). |
| I-614 | MS m/z 470.2 [M + H]⁺; ¹H NMR (400 MHz, DMSO-d6) δ 7.97 (s, 1H), 7.62 (d, |
| | J = 8.3 Hz, 1H), 7.48-7.36 (m, 1H), 7.28-7.16 (m, 4H), 6.95-6.93 (m, 1H), |
| | 6.46 (d, J = 3.2 Hz, 1H)., 3.78-3.53 (m, 3H), 3.13-2.74 (m, 1H), 2.21-1.94 |
| | (m, 3H), 1.88-1.39 (m, 4H), 1.20-0.91 (m, 2H). |
| I-616 | MS m/z: 422.1 [M + H]⁺; ¹H NMR (400 MHz, MeOD) δ 7.79 (dd, J = 2.8, 1.1 |
| | Hz, 1H), 6.98-6.92 (m, 1H), 6.85 (t, J = 73.2 Hz, 1H), 6.61-6.49 (m, 3H), |
| | 4.59-4.38 (m, 1H), 3.27-3.18 (m, 1H), 2.87-2.73 (m, 1H), 2.61-2.48 (m, |
| | 2H), 2.38-2.22 (m, 2H), 2.17-2.05 (m, 1H), 1.94-1.84 (m, 1H), 1.84-1.57 |
| | (m, 2H), 1.15 (t, J = 7.2 Hz, 3H). NH and OH not observed. |
| I-618 | MS m/z: 395.2 [M + H]⁺; ¹H NMR (400 MHz, MeOD) δ 7.79 (dd, J = 2.8, 1.1 |
| | Hz, 1H), 6.95-6.91 (m, 1H), 6.81 (t, J = 73.6 Hz, 1H), 6.56-6.49 (m, 3H), |
| | 4.38-4.17 (m, 1H), 2.72-2.60 (m, 2H), 2.34-2.18 (m, 2H), 1.43 (s, 3H). NH |
| | and OH not observed |
| I-621 | MS m/z: 463.1, 365.1 [M + H]⁺; ¹H NMR (400 MHz, METHANOL-d4) δ 7.33 |
| | (d, J = 2.0 Hz, 1H), 6.95-6.97 (m, 1H), 6.88-6.91 (m, 2H), 6.77 (dd, J = 2.0 Hz, |
| | 10.8 Hz, 1H), 4.10(s, 1H), 4.28-4.29(m, 1H), 4.05-4.09 (m, 1H), 3.87-3.97 (m, |
| | 1H), 3.81-3.87 (m, 1H), 3.50-3.56 (m, 1H), 2.10-2.17(m, 1H), 1.95-2.02(m, |
| | 2H). NH and OH not observed |
| I-629 | MS m/z 373.2 [M + H]⁺; ¹H NMR(400 MHz, CD₃OD) δ 7.77 (d, J = 1.8 Hz, 1H), |
| | 6.94-6.90 (m, 1H), 6.56 (s, 1H), 6.33 (dd, J = 10.3, 2.1 Hz, 2H), 4.23 (dd, J = |
| | 16.1, 7.9 Hz, 1H), 4.05 (q, J = 7.0 Hz, 2H), 2.69-2.61 (m, 2H), 2.25 (t, J = 10.2 |
| | Hz, 2H), 1.41-1.28 (m, 6H). NH and OH not observed |
| I-630 | MS m/z 386.3 [M + H]⁺; ¹H NMR (400 MHz, CD₃OD) δ 7.78 (dd, J = 2.9, 1.1 |
| | Hz, 1H), 6.99-6.80 (m, 1H), 6.64-6.49 (m, 1H), 6.40-6.24 (m, 2H), 4.53- |
| | 4.37 (m, 1H), 4.05 (q, J = 7.0 Hz, 2H), 3.13 (s, 1H), 2.71 (s, 1H), 2.35 (s, 3H), |
| | 2.30 (s, 2H), 2.06 (s, 1H), 1.87 (dd, J = 11.4, 6.6 Hz, 1H), 1.75 (dd, J = 9.9, 3.8 |
| | Hz, 1H), 1.63 (s, 1H), 1.40 (t, J = 7.0 Hz, 3H). NH and OH not observed |
| I-636 | MS m/z 366.3 [M + H] ; H NMR (400 MHz, METHANOL-d4) δ 8.01-7.86 |
| | (m, 1H), 7.02 (br s, 1H), 6.78-6.64 (m, 2H), 6.58 (br s, 1H), 4.88-4.65 (m, |
| | 1H), 4.16-3.89 (m, 1H), 3.28-2.85 (m, 5H), 2.38-2.30 (m, 4H), 2.18-1.88 |
| | (m, 6H), 1.34-1.30 (m, 3H); 2H (NH and OH) wasn't observed |

### Example 41a

### Preparation of Compounds I-413 and I-385

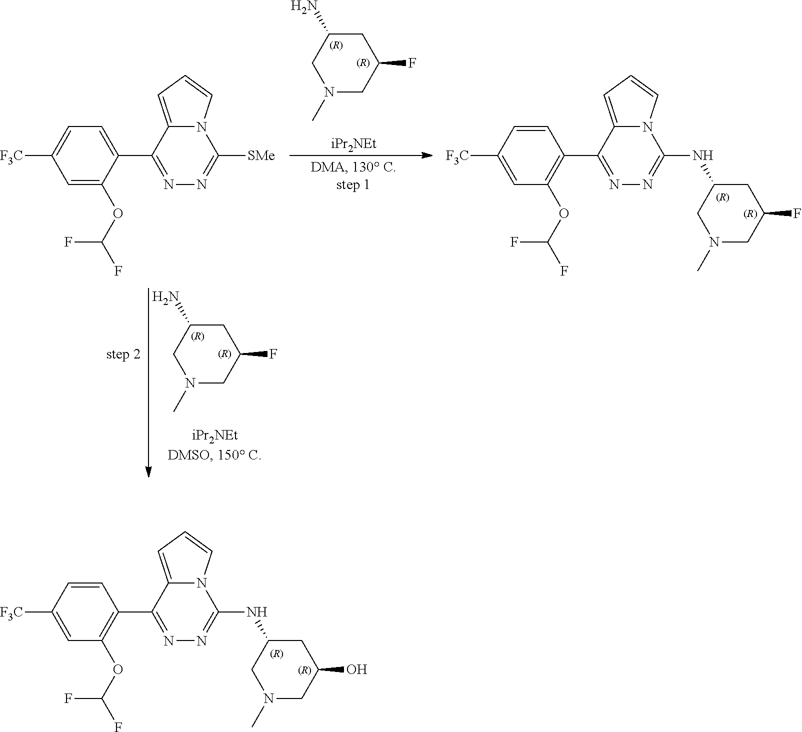

### Step 1: 1-[2-(Difluoromethoxy)-4-(trifluoromethyl)phenyl]-N-[(3R,5R)-5-fluoro-1-methyl-3-piperidyl]pyrrolo[1,2-d][1,2,4]triazin-4-amine

A mixture of 1-[2-(difluoromethoxy)-4-(trifluoromethyl)phenyl]-4-methylsulfanyl-pyrrolo[1,2-d][1,2,4]triazine (prepared as Example 32, step 1-2, 0.070 g, 0.19 mmol) and (3R,5R)-5-fluoro-1-methyl-piperidin-3-amine dihydrochloride (0.077 g, 0.37 mmol) in DMA (0.19 mL) and iPr₂NEt (0.20 mL, 1.2 mmol) was heated to 130° C. for 8 h. The reaction was cooled to rt and diluted with DCM/iPrOH (9:1). The solution was washed with brine, dried (Na₂SO₄), filtered, and concentrated. Purification by chromatography on SiO₂ (MeOH:DCM, 0 to 10%) followed by reverse phase chromatography (0.1% formic acid in MeCN:0.1% formic acid in H₂O, 5 to 100%) gave a white solid (0.037 g, 43%). MS m/z 460.1 [M+H]⁺; ¹H NMR (400 MHz, DMSO-d₆) δ 7.95 (s, 1H), 7.80 (q, J=7.9 Hz, 2H), 7.70 (s, 1H), 7.41 (t, J=61.2 Hz, 1H), 7.38 (s, 1H), 6.97 (s, 1H), 6.50 (s, 1H), 5.00 (d, J=45.28 Hz, 1H), 4.69-4.56 (m, 1H), 3.13-3.03 (m, 1H), 3.00-2.87 (m, 1H), 2.35-2.11 (m, 5H), 2.09-1.98 (m, 1H), 1.89-1.66 (m, 1H).

### Step 2. (3R,5R)-5-[[1-[2-(Difluoromethoxy)-4-(trifluoromethyl)phenyl]pyrrolo[1,2-d][1,2,4]triazin-4-yl]amino]-1-methyl-piperidin-3-ol

A mixture of 1-[4-cyclopropyl-2-fluoro-6-(methoxymethoxy)phenyl]-4-methylsulfanyl-pyrrolo[1,2-d][1,2,4]triazine (prepared as Example 32, step 1-2, 0.070 g, 0.19 mmol) and (3R,5R)-5-fluoro-1-methyl-piperidin-3-amine dihydrochloride (0.096 g, 0.47 mmol) in DMSO (0.31 mL) and iPr₂NEt (0.13 mL, 0.75 mmol) was heated to 150° C. for 17 h. The reaction was cooled to rt and diluted with DCM/iPrOH (9:1). The solution was washed with water, brine, dried (Na₂SO₄), filtered and concentrated. Purification by chromatography on SiO₂ (MeOH:DCM, 0 to 10%) followed by reverse phase chromatography (0.1% formic acid in MeCN:0.1% formic acid in H₂O, 5 to 100%) gave a white solid (0.026 g, 30%). MS m/z 440.4 [M−H₂O+H]⁺; ¹H NMR (400 MHz, CD₃OD) δ 7.93 (s, 1H), 7.89-7.71 (m, 2H), 7.67 (s, 1H), 6.99 (t, J=73.0 Hz, 1H), 6.91 (s, 1H), 6.67 (s, 1H), 4.64 (br s, 1H), 4.09 (br s, 1H), 3.42 (d, J=12.26 Hz, 1H), 3.26 (d, J=12.63 Hz, 1H), 2.82-2.70 (m, 2H), 2.45 (s, 3H), 2.36-2.17 (m, 2H); 2 Hs not observed (NH and OH).

### Example 41b

### Preparation of Compounds I-445 and I-387

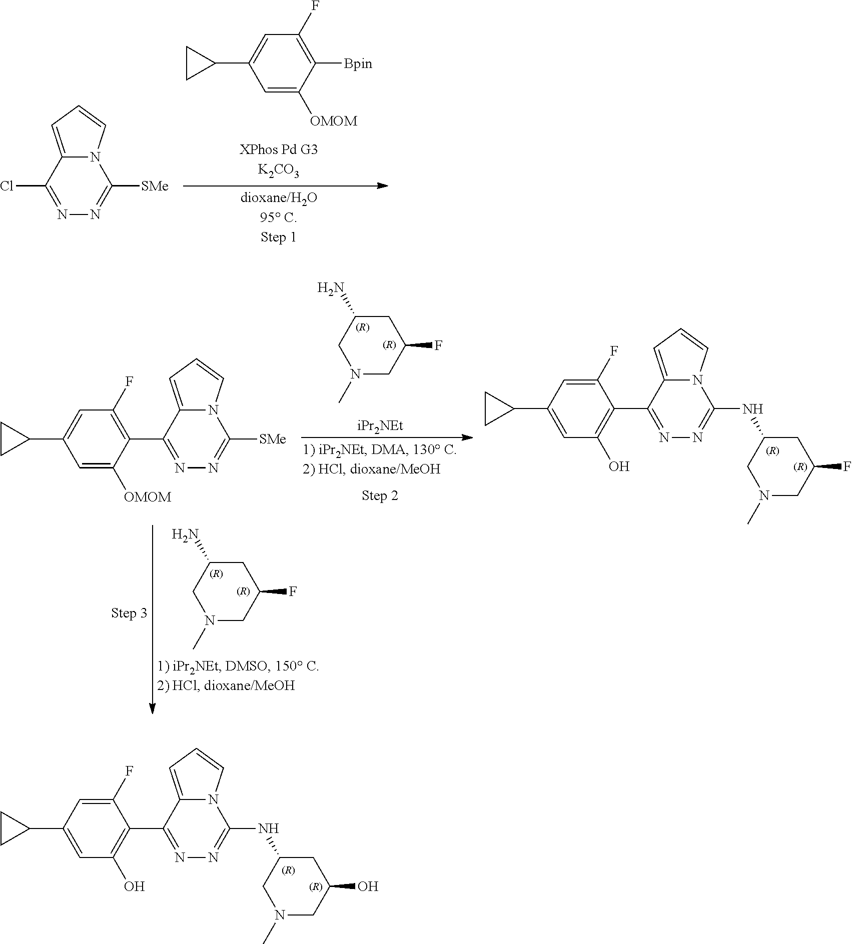

### Step 1: 1-[4-Cyclopropyl-2-fluoro-6-(methoxymethoxy)phenyl]-4-methylsulfanyl-pyrrolo[1,2-d][1,2,4]triazine

1-Chloro-4-methylsulfanyl-pyrrolo[1,2-d][1,2,4]triazine (Intermediate 2d, 0.500 g, 2.50 mmol), 2-[4-cyclopropyl-2-fluoro-6-(methoxymethoxy)phenyl]-4,4,5,5-tetramethyl-1,3,2-dioxaborolane (prepared according to the procedure of Intermediate 5d, 1.21 g, 3.76 mmol), and XPhos Pd G3 (0.216 g, 0.250 mmol) were placed in a vial and evacuated and refilled with Ar. Dioxane (12.5 mL) and aq. K₂CO₃ (2M, 0.3.8 mL, 7.51 mmol) were added and the solution was sparged with Ar for 5 min. The reaction was heated to 95° C. for 15 h. The reaction was cooled to rt and diluted with EtOAc. The solution was washed with water and brine. The organic phase was dried (Mg₂SO₄), filtered, and concentrated. Purification by chromatography on SiO₂ (EtOAc:hexanes, 0 to 60%) gave a tan solid (0.36 g, 40%). MS m/z 360.1 [M+H]⁺.

### Step 2. 5-Cyclopropyl-3-fluoro-2-[4-[[(3R,5R)-5-fluoro-1-methyl-3-piperidyl]amino]-pyrrolo[1,2-d][1,2,4]triazin-1-yl]phenol

A mixture of 1-[4-cyclopropyl-2-fluoro-6-(methoxymethoxy)phenyl]-4-methylsulfanyl-pyrrolo[1,2-d][1,2,4]triazine (0.070 g, 0.19 mmol) and (3R,5R)-5-fluoro-1-methyl-piperidin-3-amine dihydrochloride (0.080 g, 0.39 mmol) in DMA (0.19 mL) and iPr₂NEt (0.20 mL, 1.2 mmol) was heated to 130° C. for 8 h. The reaction was cooled to rt and diluted with DCM/iPrOH (9:1). The solution was washed with water, brine, dried (Na₂SO₄), filtered, and concentrated. The resulting oil was dissolved in MeOH (0.5 mL) and added HCl/dioxane (4M, 1.0 mL). The reaction was stirred for 1 h. The mixture was concentrated, and the resulting residue was dissolved in DCM/iPrOH (9:1). The solution was washed with sat. NaHCO₃ and brine, dried (Na₂SO₄), filtered, and concentrated. Purification by chromatography on SiO₂ (MeOH:DCM, 0 to 10%) followed by reverse phase chromatography (0.1% formic acid in MeCN:0.1% formic acid in H₂O, 5 to 100%) gave a white solid (0.010 g, 13%). MS m/z 400.6 [M+H]⁺; ¹H NMR (400 MHz, DMSO-d₆) δ 7.87 (s, 1H), 7.25 (d, J=6.8 Hz, 1H), 6.89 (s, 1H), 6.54 (s, 1H), 6.47 (d, J=10.8 Hz, 1H), 6.33 (s, 1H), 5.01 (d, J=47.0 Hz, 1H), 4.66-4.53 (m, 1H), 3.08 (d, J=8.8 Hz, 1H), 2.93 (t, J=11.2 Hz, 1H), 2.34-2.11 (m, 5H), 2.08-1.98 (m, 1H), 1.97-1.87 (m, 1H), 1.87-1.65 (m, 1H), 1.03-0.99 (m, 2H), 0.76-0.66 (m, 2H). 1H not observed (OH).

### Step 3: (3R,5R)-5-[[1-(4-Cyclopropyl-2-fluoro-6-hydroxy-phenyl)pyrrolo[1,2-d][1,2,4]triazin-4-yl]amino]-1-methyl-piperidin-3-ol formic acid salt

A mixture of 1-[4-cyclopropyl-2-fluoro-6-(methoxymethoxy)phenyl]-4-methylsulfanyl-pyrrolo[1,2-d][1,2,4]triazine (0.119 g, 0.331 mmol) and (3R,5R)-5-fluoro-1-methyl-piperidin-3-amine dihydrochloride (0.153 g, 0.745 mmol) in DMSO (0.47 mL) and iPr₂NEt (0.23 mL, 1.32 mmol) was heated to 150° C. for 12 h. The reaction was cooled to rt and diluted with DCM/iPrOH (9:1). The solution was washed with water, brine, dried (Na₂SO₄), filtered, and concentrated. The resulting oil was dissolved in MeOH (0.5 mL) and added HCl/dioxane (4M, 1.0 mL). The reaction was stirred for 1 h. The mixture was concentrated, and the resulting residue was dissolved in DCM/iPrOH (9:1). The solution was washed with sat. NaHCO₃ and brine, dried (Na₂SO₄), filtered, and concentrated. Purification by chromatography on SiO₂ (MeOH:DCM, 0 to 10%) followed by reverse phase chromatography (0.1% formic acid in MeCN:0.1% formic acid in H₂O, 5 to 100%) gave a white solid (0.012 g, 8%). MS m/z 380.5 [M−H₂O+H]⁺; ¹H NMR (500 MHz, CD₃OD) δ 8.49 (br s, 1H, formic acid), 7.81 (s, 1H), 6.83-6.76 (m, 1H), 6.55-6.43 (m, 3H), 4.53-4.47 (m, 1H), 4.00-3.94 (m, 1H), 3.29 (d, J=11.7 Hz, 1H), 3.15 (d, J=11.1 Hz, 1H), 2.60-2.47 (m, 2H), 2.34 (s, 3H), 2.24-2.09 (m, 2H), 1.97-1.86 (m, 1H), 1.11-0.99 (m, 2H), 0.78-0.71 (m, 2H); 3 Hs not observed (2 OH, NH).

### Example 42

### Preparation of Compound I-486

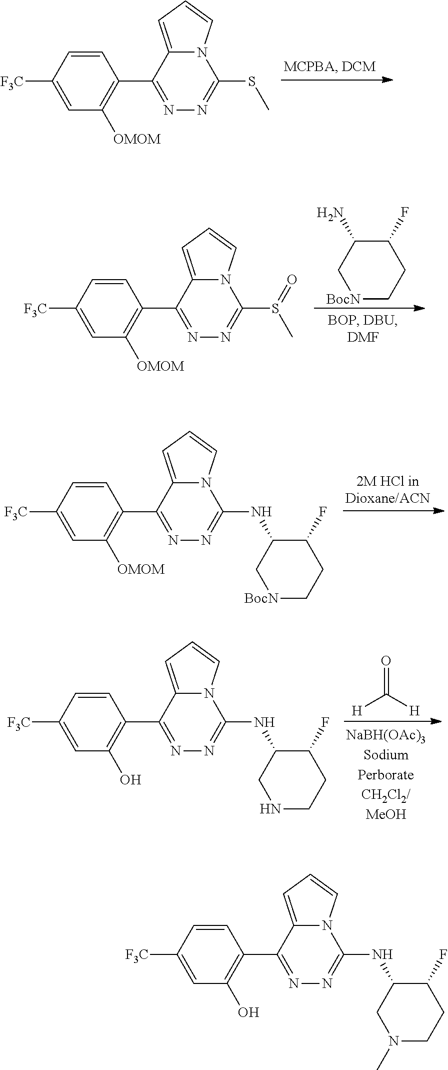

### Step 1. 1-(2-(Methoxymethoxy)-4-(trifluoromethyl)phenyl)-4-(methylsulfinyl)pyrrolo[1,2-d][1,2,4]triazine

mCPBA (70%, 111 mg, 0.450 mmol, 1.5 eq) was added to a mixture of 1-(2-(methoxymethoxy)-4-(trifluoromethyl)phenyl)-4-(methylthio)pyrrolo[1,2-d][1,2,4]triazine (111 mg, 0.300 mmol, 1 eq., prepared in a manner analogous to Example 38, step 1, using intermediate 5a in place of 2-[2-fluoro-6-(methoxymethoxy)-4-methyl-phenyl]-4,4,5,5-tetramethyl-1,3,2-dioxaborolane) in DCM at 0° C. The reaction mixture was allowed to slowly return to room temperature and stirred for 30 min. After 30 min, the reaction mixture was diluted with a small amount of NaHCO₃ (sat. aq.), extracted several times with DCM and the combined organic extracts were dried over Na₂SO₄ and concentrated in vacuo. The crude residue was used without further purification. MS m/z 386.4 [M+H]⁺.

### Step 2. tert-Butyl (3S,4R)-4-fluoro-3-((1-(2-(methoxymethoxy)-4-(trifluoromethyl)phenyl)pyrrolo[1,2-d][1,2,4]triazin-4-yl)amino)piperidine-1-carboxylate

N,N-diisopropylethylamine (0.235 mL, 1.35 mmol, 4.5 eq) was added to a solution of 1-(2-(methoxymethoxy)-4-(trifluoromethyl)phenyl)-4-(methylsulfinyl)pyrrolo[1,2-d][1,2,4]triazine (116 mg, 0.300 mmol, 1 eq) and tert-butyl (3S,4R)-3-amino-4-fluoropiperidine-1-carboxylate (131 mg, 0.600 mmol, 2 eq) in DMA (0.6 mL) and stirred at 50° C. for 1 day. The reaction mixture was allowed to slowly return to room temperature and stirred for 30 min. After completion, the reaction was diluted with EtOAc and NaHCO₃(sat. aq.). The product was extracted several times with EtOAc and the combined organic extracts were dried over Na₂SO₃ and concentrated in vacuo. The crude residue was purified by silica gel column chromatography eluting with 1:100 to 20:80 MeOH:DCM to afford tert-butyl (3S,4R)-4-fluoro-3-((1-(2-(methoxymethoxy)-4-(trifluoromethyl)phenyl)pyrrolo[1,2-d][1,2,4]triazin-4-yl)amino)piperidine-1-carboxylate as an impure oil which was used without further purification. MS m/z 540.6 [M+H]⁺.

### Step 3. 2-(4-(((3S,4R)-4-Fluoropiperidin-3-yl)amino)pyrrolo[1,2-d][1,2,4]triazin-1-yl)-5-(trifluoromethyl)phenol formic acid salt

The mixture from step 2 was suspended in ACN (2 mL) and 4 M HCl in Dioxane (1 mL) and stirred at room temperature for 2 h. Upon reaction completion solvents were removed in vacuo. The residue was concentrated several times from dichloromethane to remove excess HCl. The combined organic extracts were dried over sodium sulfate and the crude material was purified by flash column chromatography 0:100 to 10:90 (10% NH₄OH in MeOH):DCM to afford 2-(4-(((3S,4R)-4-fluoropiperidin-3-yl)amino)pyrrolo[1,2-d][1,2,4]triazin-1-yl)-5-(trifluoromethyl)phenol followed by C18 reverse phase Prep-HPLC eluting with ACN:Water with formic acid as the modifier. (35 mg, 30% over three steps). MS m/z 396.5 [M+H]⁺; ¹H NMR (400 MHz, Methanol-d₄) δ 8.47 (s, 1H), 7.91-7.78 (m, 1H), 7.56-7.29 (m, 4H), 5.43-5.19 (m, 1H), 5.01-4.87 (m, 1H), 3.78-3.63 (m, 1H), 3.55-3.36 (m, 2H), 3.32-3.20 (m, 1H), 2.54-2.40 (m, 1H), 2.38-2.14 (m, 1H), 3 exchangeable protons not observed.

### Step 4. 2-(4-(((3S,4R)-4-Fluoro-1-methylpiperidin-3-yl)amino)pyrrolo[1,2-d][1,2,4]triazin-1-yl)-5-(trifluoromethyl)phenol

A mixture of 2-(4-(((3S,4R)-4-fluoropiperidin-3-yl)amino)pyrrolo[1,2-d][1,2,4]triazin-1-yl)-5-(trifluoromethyl)phenol (33 mg, 0.083 mmol, 1 eq) and sodium perborate tetrahydrate (39 mg, 0.25o mmol, 3 eq) in DCM (1 mL) and methanol (200 μL) was stirred for 30 min at 0° C. After 30 min, formaldehyde (37 wt % in water) (18.6 μL, 0.250 mmol, 3 eq) followed by sodium triacetoxyborohydride (53 mg, 0.250 mmol, 3 eq). The reaction was stirred at 0° C. for 1 min. The ice bath was removed, and the reaction mixture was stirred for 2 min. The reaction was quenched by addition a small amount of NaHCO₃ (sat, aq) and a small amount of water followed by extraction with DCM. The combined organic extracts were dried over sodium sulfate and the crude material was purified by C18 reverse phase Prep-HPLC eluting with ACN:Water with formic acid as the modifier. After reverse phase purification, solvents were removed to afford 2-(4-(((3S,4R)-4-fluoro-1-methylpiperidin-3-yl)amino)pyrrolo[1,2-d][1,2,4]triazin-1-yl)-5-(trifluoromethyl)phenol (17.6 mg, 52%). MS m/z 410.5 [M+H]⁺; ¹H NMR (400 MHz, Methanol-d₄) δ 8.21-8.09 (i, 1H), 8.07-7.96 (m, 1H), 7.36-7.19 (i, 3H), 7.17-7.04 (1H), 5.27-5.03 (i, 1H), 4.81-4.61 (m, 1H), 3.20-3.08 (m, 1H), 2.96-2.80 (m, 1H), 2.79-2.66 (m, 1H), 2.64-2.38 (i, 4H), 2.32-1.94 (2H), NH and OH not observed.

The compounds below were prepared according to the procedure of Example 42 by substituting the appropriate starting materials, reagents and reaction conditions.)

| Compound | Spectral Data |
|---|---|
| I-408 | MS m/z 312.0 [M + H]⁺; ¹H NMR (CD₃OD) δ: 8.43-8.46 (m, 1H), 7.76 (d, J = 8.5 |
| | Hz, 1H), 7.51 (d, J = 4.1 Hz, 1H), 7.42 (dd, J = 4.1, 2.9 Hz, 1H), 7.02-7.09 (m, |
| | 2H), 5.26-5.42 (m, 1H), 4.84-4.90 (m, 1H), 3.85 (br dd, J = 12.1, 3.7 Hz, 1H), |
| | 3.68-3.75 (m, 1H), 3.36-3.50 (m, 1H), 3.15-3.30 (m, 1H), 2.66-2.78 (m, 1H), |
| | 2.19-2.40 (m, 1H). OH and NH not observed |
| I-409 | MS m/z 426.0 [M + H]⁺; ¹H NMR (CD₃OD) δ: 8.07 (d, J = 8.7 Hz, 1H), 7.88 (br s, |
| | 1H), 7.25 (d, J = 3.7 Hz, 1H), 7.08 (br s, 1H), 6.86-6.93 (m, 2H), 4.96-5.09 (m, |
| | 1H), 4.72-4.82 (m, 1H), 3.18-3.31 (m, 1H), 3.05 (br t, J = 10.8 Hz, 1H), 2.31-2.48 |
| | (m, 5H), 2.22 (br t, J = 10.2 Hz, 1H), 1.80-1.99 (m, 1H). OH and NH not observed |
| I-410 | MS m/z 410.4 [M + H]⁺; ¹H NMR (400 MHz, Methanol-d4) δ 8.22-8.05 (m, |
| | 1H), 8.00-7.79 (m, 1H), 7.38-7.15 (m, 3H), 7.13-7.00 (m, 1H), 5.16-4.91 |
| | (m, 1H), 4.80-4.67 (m, 1H), 3.42-3.31 (m, 1H), 3.19-2.96 (m, 1H), 2.55- |
| | 2.19 (m, 6H), 2.06-1.77 (m, 1H). OH and NH not observed. |
| I-439 | MS m/z 478.6 [M + H]⁺; ¹H NMR (CD₃OD) δ: 7.68-7.80 (m, 3H), 7.60 (s, 1H), |
| | 6.94 (t, J = 72 Hz, 1H), 6.83 (bs, 1H), 4.92-5.09 (m, 1H), 4.75-4.83 (m, 1H), |
| | 3.23-3.29 (m, 1H), 3.03 (br t, J = 10.9 Hz, 1H), 2.30-2.50 (m, 5H), 2.20-2.29 (m, |
| | 1H), 1.78-1.99 (m, 1H). NH not observed. |
| I-444 | MS m/z 374.1 [M + H]⁺; ¹H NMR (400 MHz, DMSO-d6) δ 7.88 (s, 1H), 7.24 (d, |
| | J = 7.3 Hz, 1H), 6.89 (s, 1H), 6.65-6.56 (m, 2H), 6.32 (s, 1H), 5.01 (d, J = |
| | 46.9 Hz, 1H), 4.68-4.50 (m, 1H), 3.08 (d, J = 12.26 Hz, 1H), 3.00-2.86 (m, |
| | 1H), 2.31 (s, 3H), 2.28-2.18 (m, 4H), 2.17-2.09 (m, 1H), 2.08-1.97 (m, |
| | 1H), 1.89-1.65 (m, 1H), 1H not observed (OH or NH) |
| I-468 | MS m/z 462.4 [M + H]⁺; ¹H NMR (400 MHz, DMSO-d6) δ 8.25 (s, 1H), 8.21 (d, |
| | J = 7.9 Hz, 1H), 7.98 (s, 1H), 7.90 (d, J = 7.3 Hz, 1H), 7.41 (d, J = 7.0 Hz, 1H), |
| | 6.96 (s, 1H), 6.40 (s, 1H), 5.01 (d, J = 46.5 Hz, 1H), 4.70-4.55 (m, 1H), 3.14-3.03 |
| | (m, 1H), 3.00-2.88 (m, 1H), 2.35-2.11 (m, 5H), 2.09-1.98 (m, 1H), |
| | 1.89-1.65 (m, 1H). |
| I-478 | MS m/z 410.6 [M + H]⁺; ¹H NMR (400 MHz, Methanol-d4) δ 8.20-8.05 (m, |
| | 1H), 7.98-7.85 (m, 1H), 7.31-7.17 (m, 3H), 7.14-7.04 (m, 1H), 4.84-4.56 |
| | (m, 2H), 3.24-3.11 (m, 1H), 2.87-2.74 (m, 1H), 2.45-2.30 (m, 5H), 2.28-2.13 |
| | (m, 1H), 2.05-1.92 (m, 1H). OH and NH not observed. |
| I-481 | MS m/z 394.2, 396.2 [M + H]⁺; ¹H NMR (400 MHz, CD₃OD) δ 7.92 (s, 1H), |
| | 6.96 (d, J = 3.5 Hz, 1H), 6.86 (d, J = 9.5 Hz, 2H), 6.65 (d, J = 3.5 Hz, 1H), |
| | 5.33-5.13 (m, 1H), 3.78 (s, 1H), 3.55 (d, J = 12.6 Hz, 1H), 3.43 (d, J = 12.4 Hz, |
| | 1H), 3.16-2.95 (m, 1H), 2.80 (s, 3H), 2.57 (s, 1H), 2.31 (t, J = 12.4 Hz, 1H), |
| | 2.20-1.96 (m, 1H). missing NH and OH peak |
| I-521 | MS m/z 476.6 [M + H]⁺; ¹H NMR (400 MHz, Methanol-d4) δ 7.85-7.77 (m, |
| | 1H), 7.74-7.63 (m, 1H), 7.41-7.33 (m, 1H), 7.33-7.28 (m, 1H), 7.07-6.65 |
| | (m, 2H), 6.62-6.53 (m, 1H), 5.15-4.94 (m, 1H), 4.84-4.74 (m, 1H), 3.44-3.34 |
| | (m, 1H), 3.20-3.05 (m, 1H), 2.61-2.39 (m, 5H), 2.39-2.25 (m, 1H), |
| | 2.03-1.80 (m, 1H). NH peak not observed |
| I-535 | MS m/z: 476.2 [M + H]⁺; ¹H NMR (400 MHz, DMSO-d6) δ 8.25 (s, 1H), 8.21 |
| | (d, J = 8.6 Hz, 1H), 7.98 (d, J = 2.0 Hz, 1H), 7.91 (d, J = 8.0 Hz, 1H), 7.40 (d, |
| | J = 7.4 Hz, 1H), 7.02-6.88 (m, 1H), 6.40 (d, J = 3.1 Hz, 1H), 5.02 (d, J = 47.9 |
| | Hz, 1H), 4.69-4.55 (m, 1H), 3.20-3.12 (m, 1H), 3.08-2.98 (m, 1H), 2.47-2.41 |
| | (m, 2H), 2.37-2.14 (m, 2H), 2.12-2.02 (m, 1H), 1.91-1.69 (m, 1H), |
| | 1.02 (t, J = 7.1 Hz, 3H). |
| I-538 | MS m/z: 474.2 [M + H]⁺; ¹HNMR (400 MHz, DMSO-d6) δ: 7.95 (d, J = 1.8 |
| | Hz, 1H), 7.85-7.76 (m, 2H), 7.70 (s, 1H), 7.40 (t, J = 66.0 Hz, 1H), 7.38 (s, |
| | 1H), 7.02-6.92 (m, 1H), 6.50 (d, J = 3.8 Hz, 1H), 5.01 (d, J = 47.2 Hz, 1H), |
| | 4.68-4.55 (m, 1H), 3.18 (d, J = 8.2 Hz, 1H), 3.09-2.96 (m, 1H), 2.47-2.40 |
| | (m, 2H), 2.36-2.29 (m, 1H), 2.23-2.20 (m, 1H), 2.07-2.04 m, 1H), 1.90-1.71 |
| | (m, 1H), 1.02 (t, J = 7.0 Hz, 3H). |
| I-571 | MS m/z 388.2 [M + H]⁺; ¹H NMR (400 MHz, DMSO-d6) δ 9.92 (s, 1H), 7.87 |
| | (d, J = 1.8 Hz, 1H), 7.23 (d, J = 7.8 Hz, 1H), 6.93-6.83 (m, 1H), 6.62 (s, 1H), |
| | 6.60 (d, J = 10.4 Hz, 1H), 6.32 (d, J = 3.8 Hz, 1H), 5.01 (d, J = 47.4 Hz, 1H), |
| | 4.60 (br s, 1H), 3.18 (d, J = 7.8 Hz, 1H), 3.03 (t, J = 11.8 Hz, 1H), 2.47-2.40 |
| | (m, 2H), 2.37-2.32 (m, 1H), 2.30 (s, 3H), 2.28-2.12 (m, 1H), 2.05 (t, J = |
| | 10.0 Hz, 1H), 1.91-1.69 (m, 1H), 1.02 (t, J = 7.1 Hz, 3H). |
| I-572 | MS m/z 414.3 [M + H]⁺; ¹H NMR (400 MHz, DMSO-d6) δ 9.93 (s, 1H), 7.87 |
| | (d, J = 1.8 Hz, 1H), 7.23 (d, J = 7.7 Hz, 1H), 6.93-6.86 (m, 1H), 6.54 (s, 1H), |
| | 6.48 (d, J = 11.0 Hz, 1H), 6.34 (d, J = 3.7 Hz, 1H), 5.01 (d, J = 47.6 Hz, 1H), |
| | 4.59 (br s, 1H), 3.17 (d, J = 8.9 Hz, 1H), 3.04 (t, J = 12.4 Hz, 1H), 2.47-2.39 |
| | (m, 2H), 2.36-2.13 (m, 2H), 2.06 (t, J = 10.2 Hz, 1H), 1.91-1.68 (m, 1H), |
| | 1.02 (t, J = 7.1 Hz, 3H). |
| I-596 | MS m/z 428.3 [M + H]⁺; ¹H NMR (400 MHz, Methanol-d4) δ 8.34 (s, 1H, |
| | formic acid proton), 7.82-7.54 (m, 2H), 7.41-7.09 (m, 2H), 6.95-6.72 (m, |
| | 1H), 5.24-4.99 (m, 1H), 4.84-4.69 (m, 1H), 3.57-3.41 (m, 1H), 3.31-3.19 |
| | (m, 1H), 2.80-2.33 (m, 6H), 2.10-1.81 (m, 1H). NH and OH not observed |
| I-597 | MS m/z 408.3 [M + H—H₂O]⁺; ¹H NMR (400 MHz, Methanol-d4) δ 8.40 (s, 1H, |
| | formic acid proton), 7.72-7.59 (m, 1H), 7.56-7.48 (m, 1H), 7.28-7.19 (m, |
| | 1H), 7.19-7.14 (m, 1H), 6.60-6.54 (m, 1H), 4.49-4.38 (m, 1H), 3.97-3.87 |
| | (m, 1H), 3.40-3.32 (m, 1H), 3.19-3.09 (m, 1H), 2.59-2.46 (m, 2H), 2.35 (s, |
| | 3H), 2.26-2.02 (m, 2H). NH and OH not observed |
| I-598 | MS m/z 458.3 [M + H]⁺. ¹H NMR (400 MHz, Methanol-d4) δ 8.41 (s, 1H, |
| | formic acid proton), 7.79-7.49 (m, 2H), 7.04-6.69 (m, 3H), 5.23-4.97 (m, |
| | 1H), 4.84-4.67 (m, 1H), 3.64-3.43 (m, 1H), 3.40-3.32 (m, 1H), 2.93-2.58 |
| | (m, 3H), 2.55-2.36 (m, 2H), 2.09-1.84 (m, 1H), 1.31-1.14 (m, 3H). NH and |
| | OH not observed |
| I-603 | MS m/z 472.3 [M + H—H₂O]⁺; ¹H NMR (400 MHz, Methanol-d4) δ 7.81-7.74 |
| | (m, 1H), 7.72-7.61 (m, 2H), 7.60-7.54 (m, 1H), 6.96 (t, J = 73.1 Hz, 1H), |
| | 6.61-6.52 (m, 1H), 4.46-4.36 (m, 1H), 3.94-3.84 (m, 1H), 3.39-3.31 (m, |
| | 1H), 3.23-3.13 (m, 1H), 2.71-2.36 (m, 4H), 2.25-2.01 (m, 2H), 1.08-0.97 |
| | (m, 3H). NH and OH not observed |
| I-605 | MS m/z 438.3 [M + H—H₂O]⁺; ¹H NMR (400 MHz, Methanol-d4) δ 7.66-7.59 |
| | (m, 1H), 7.48-7.39 (m, 1H), 6.89-6.77 (m, 2H), 6.56-6.47 (m, 1H), 4.43-4.34 |
| | (m, 1H), 3.91-3.83 (m, 1H), 3.38-3.30 (m, 1H), 3.23-3.14 (m, 1H), |
| | 2.61-2.37 (m, 4H), 2.21-1.99 (m, 2H), 1.10-0.96 (m, 3H). NH and OH not |
| | observed |
| I-615 | MS m/z 458.2 [M + H]⁺; ¹H NMR (400 MHz, DMSO-d6) δ 7.92 (d, J = 1.8 Hz, |
| | 1H), 7.62 (d, J = 8.3 Hz, 1H), 7.43 (d, J = 15.9 Hz, 1H), 7.31 (d, J = 7.9 Hz, |
| | 1H), 7.25-7.18 (m, 3H), 6.98-6.93 (m, 1H), 6.45 (d, J = 2.8 Hz, 1H), 5.01 (d, |
| | J = 47.3 Hz, 1H), 4.70-4.52 (m, 1H), 3.08 (d, J = 10.7 Hz, 1H), 3.02-2.88 |
| | (m, 1H), 2.36-2.11 (m, 5H), 2.06-1.98 (m, 1H), 1.87-1.65 (m, 1H). |
| I-617 | MS m/z: 426.2 [M + H]⁺; ¹H NMR (400 MHz, MeOD) δ 7.77 (dd, J = 2.9, 1.1 |
| | Hz, 1H), 6.98-6.93 (m, 1H), 6.83 (d, J = 73.5 Hz, 1H), 6.61-6.51 (m, 3H), |
| | 5.07-4.90 (m, 1H), 4.81-4.72 (m, 1H), 3.27-3.20 (m, 1H), 3.10-2.95 (m, |
| | 1H), 2.54-2.39 (m, 2H), 2.37 (s, 3H), 2.30-2.18 (m, 1H), 1.99-1.77 (m, |
| | 1H). NH and OH not observed |
| I-619 | MS m/z: 408.0, 410.0 [M + H]⁺; ¹H NMR (400 MHz, MeOD) δ 8.28 (d, J = 1.7 |
| | Hz, 1H), 7.39-7.30 (m, 2H), 7.01 (t, J = 72.8 Hz, 1H), 6.75-6.72 (m, 1H), |
| | 6.96 (s, 1H), 5.35 (d, J = 44.0 Hz, 1H), 4.06-3.82 (m, 2H), 3.42-3.33 (m, |
| | 4H), 3.16-3.06 (m, 1H), 2.78-2.66 (m, 1H), 2.35-2.02 (m, 1H), 1.41 (t, J = |
| | 7.3 Hz, 3H). OH and NH not observed. |
| I-620 | MS m/z: 408.0 [M + H]⁺; ¹H NMR (400 MHz, MeOD) δ 8.28 (d, J = 1.7 Hz, |
| | 1H), 7.39-7.30 (m, 2H), 7.01 (dd, J = 9.5, 1.7 Hz, 1H), 6.96 (s, 1H), 5.35 (d, |
| | J = 44.0 Hz, 1H), 4.06-3.82 (m, 2H), 3.42-3.33 (m, 4H), 3.16-3.06 (m, 1H), |
| | 2.78-2.66 (m, 1H), 2.35 - 2.02 (m, 1H), 1.41 (t, J = 7.3 Hz, 3H). NH and OH |
| | not observed |
| I-631 | MS m/z: 404.1 [M + H]⁺; ¹H NMR (400 MHz, MeOD) δ 7.77 (d, J = 2.7 Hz, |
| | 1H), 6.95 (t, J = 3.3 Hz, 1H), 6.58 (s, 1H), 6.35 (d, J = 8.1 Hz, 2H), 4.99 (d, J = |
| | 46.6 Hz, 2H), 4.78 (s, 1H), 4.08 (q, J = 7.0 Hz, 2H), 3.27 (d, J = 7.9 Hz, 1H), |
| | 3.02 (t, J = 10.2 Hz, 1H), 2.44 (d, J = 11.4 Hz, 1H), 2.38 (s, 3H), 2.25 (t, J = |
| | 10.2 Hz, 1H), 1.91 (dt, J = 24.8, 11.2 Hz, 1H), 1.43 (t, J = 7.0 Hz, 3H). OH and |
| | NH not observed. |

### Example 43

### Preparation of Compound I-485

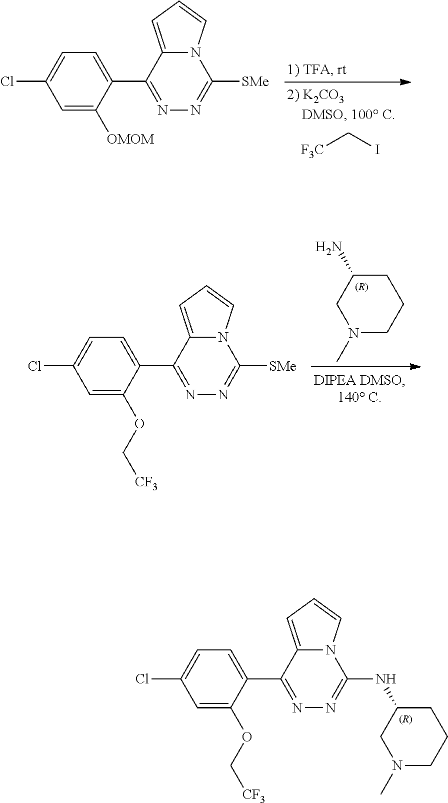

### Step 1: 1-(4-Chloro-2-(2,2,2-trifluoroethoxy)phenyl)-4-(methylthio)pyrrolo[1,2-d][1,2,4]triazine

A solution of 1-(4-chloro-2-(methoxymethoxy)phenyl)-4-(methylthio)pyrrolo[1,2-d][1,2,4]triazine (100 mg, 0.34 mmol, prepared in a manner analogous to Example 38, step 1, using 2-(4-chloro-2-(methoxymethoxy)phenyl)-4,4,5,5-tetramethyl-1,3,2-dioxaborolane in place of 2-[2-fluoro-6-(methoxymethoxy)-4-methyl-phenyl]-4,4,5,5-tetramethyl-1,3,2-dioxaborolane) in TFA (0.3 mL) was stirred at room temperature for 1 hour. Upon completion, TFA was removed under reduced pressure to afford 5-chloro-2-(4-(methylthio)pyrrolo[1,2-d][1,2,4]triazin-1-yl)phenol. The crude product was taken to the next step without further purification.

To a solution of 5-chloro-2-(4-(methylthio)pyrrolo[1,2-d][1,2,4]triazin-1-yl)phenol (110 mg, 0.38 mmol, 1 eq) in DMSO (2 mL, 0.2 M) is added 2-iodo-1,1,1-trifluoroethane (40 μL, 0.4 mmol, 1.1 eq), and potassium carbonate (110 mg, 0.8 eq, 2.1 eq). The mixture was stirred vigorously at 100° C. for 6 hours. Upon completion the mixture was diluted with ethyl acetate (10 mL) and washed with a 10% aqueous solution of Na₂CO₃ (2×10 mL), water (2×10 mL), and brine (2×10 mL). The organic layer was dried over Na₂SO₄ and concentrated in vacuo. The crude residue was purified on silica gel chromatography eluding with 0-100% ethyl acetate in hexane to afford 1-(4-chloro-2-(2,2,2-trifluoroethoxy)phenyl)-4-(methylthio)pyrrolo[1,2-d][1,2,4]triazine as an orange oil (66 mg, 46%). MS m/z 374.1, 376.1 [M+H]⁺.

### Step 2. (R)-1-(4-Chloro-2-(2,2,2-trifluoroethoxy)phenyl)-N-(1-methylpiperidin-3-yl)pyrrolo[1,2-d][1,2,4]triazin-4-amine

To a solution of 1-(4-chloro-2-(2,2,2-trifluoroethoxy)phenyl)-4-(methylthio)pyrrolo[1,2-d][1,2,4]triazine (66 mg, 0.18 mmol, 1 eq) in DMSO (300 μL, 0.55 M) was added DIPEA (100 μL, 0.57 mmol, 3.3 eq) and (3R)-1-methylpiperidin-3-amine (62 mg, 0.54 mmol, 3 eq). The resulting mixture was heated to 140° C. for 24 hours. The reaction was then cooled to room temperature, diluted with 10% i-PrOH in DCM (20 mL) and washed with H₂O (3×20 mL) and brine (3×20 mL), dried over Na₂SO₄, and concentrated in vacuo. The crude residue was purified silica gel column chromatography eluting with 0-30% methanol in DCM to afford (R)-1-(4-chloro-2-(2,2,2-trifluoroethoxy)phenyl)-N-(1-methylpiperidin-3-yl)pyrrolo[1,2-d][1,2,4]triazin-4-amine (20 mg, 28% yield). MS m/z 440.3, 442.3 [M+H]⁺. ¹H NMR (400 MHz, CD₃OD) δ 7.83 (s, 1H), 7.44 (d, J=8.2 Hz, 1H), 7.31 (s, 1H), 7.21 (d, J=8.0 Hz, 1H), 6.92 (t, J=3.3 Hz, 1H), 6.53 (d, J=3.7 Hz, 1H), 4.58 (t, J=8.5 Hz, 2H), 4.54-4.38 (m, 1H), 3.33-3.18 (m, 2H), 2.83 (d, J=11.3 Hz, 1H), 2.44 (s, 3H), 2.41-2.31 (m, 1H), 2.10 (d, J=12.4 Hz, 1H), 1.91 (d, J=14.4 Hz, 1H), 1.86-1.55 (m, 2H). NH peak not observed.

### Example 44

### Preparation of Compound I-484

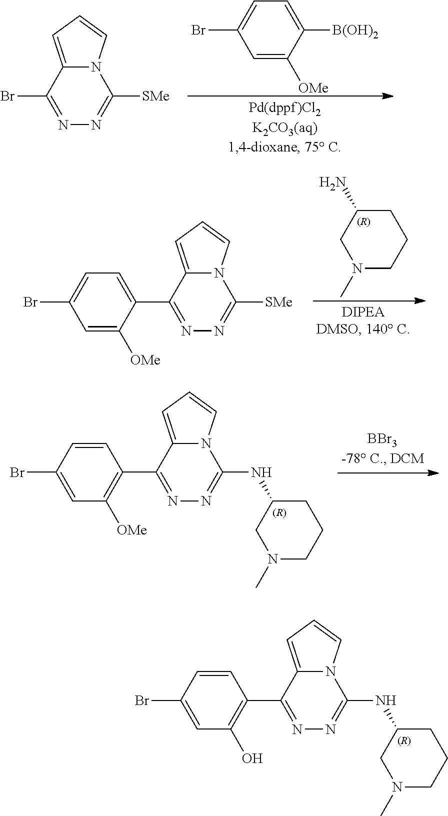

### Step 1: 1-(4-Bromo-2-methoxyphenyl)-4-(methylthio)pyrrolo[1,2-d][1,2,4]triazine

To a solution of 1-bromo-4-(methylthio)pyrrolo[1,2-d][1,2,4]triazine (Intermediate 2e, 90 mg, 0.36 mmol, 1 eq) in 1,4-dioxane (1.8 mL, 0.2 M) was added (4-bromo-2-methoxyphenyl)boronic acid (102 mg, 0.43 mmol, 1.2 eq), Pd(dppf)Cl₂ (94 mg, 0.1 mmol, 0.25 eq), and K₂CO₃ (2 M in H₂O) (550 μL, 1.1 mL, 3 eq). The resulting mixture was heated to 75° C. for 4 hours. The reaction is then cooled to room temperature, diluted with H₂O (10 mL) and the aqueous phase was extracted with EtOAc (3×15 mL). The combined organic phases were washed with brine, dried over Na₂SO₄, and concentrated in vacuo. The crude residue was purified by silica gel column chromatography eluting with 0-100% EtOAc in hexanes to afford (4-bromo-2-methoxyphenyl)-4-(methylthio)pyrrolo[1,2-d][1,2,4]triazine as a yellow oil (51 mg, 42% yield).

### Step 2. (R)-1-(4-Bromo-2-methoxyphenyl)-N-(1-methylpiperidin-3-yl)pyrrolo[1,2-d][1,2,4]triazin-4-amine

To a solution of (4-bromo-2-methoxyphenyl)-4-(methylthio)pyrrolo[1,2-d][1,2,4]triazine (51 mg, 0.15 mmol, 1 eq) in DMSO (300 uL, 0.5M) was added DIPEA (80 μL, 0.5 mmol, 3.2 eq) and (3R)-1-methylpiperidin-3-amine (34 mg, 0.3 mmol, 2 eq). The resulting mixture was heated to 140° C. for 24 hours. The reaction was then cooled to room temperature, diluted with 10% i-PrOH in DCM (20 mL) and washed with H₂O (3×20 mL) and brine (3×20 mL), dried over Na₂SO₄, and concentrated in vacuo. The crude residue was purified silica gel column chromatography eluting with 0-30% methanol in DCM to afford (R)-1-(4-bromo-2-methoxyphenyl)-N-(1-methylpiperidin-3-yl)pyrrolo[1,2-d][1,2,4]triazin-4-amine as a yellow oil (46 mg, 0.11 mmol, 72% yield). MS m/z 416.3, 418.3 [M+H]⁺.

### Step 3: (R)-5-Bromo-2-(4-((1-methylpiperidin-3-yl)amino)pyrrolo[1,2-d][1,2,4]triazin-1-yl)phenol

To a solution of afford (R)-1-(4-bromo-2-methoxyphenyl)-N-(1-methylpiperidin-3-yl)pyrrolo[1,2-d][1,2,4]triazin-4-amine (46 mg, 0.11 mmol, 1 eq) in DCM (400 uL, 0.3M) at −78′C was added boron tribromide (1.1 mL, 1.1 mmol, 10 eq). The reaction was warmed to room temperature naturally and stirred for 30 minutes. The mixture was then diluted with 10 mL of DCM, cooled to 0° C. and 10% aqueous sodium bicarbonate was added dropwise until a pH of 7 is achieved. The aqueous layer was extracted with 10% i-PrOH in DCM (3×20 mL). The combined organic layers were washed with brine, dried over Na₂SO₄, and concentrated in vacuo. The crude residue was purified on silica gel chromatography eluding with 0-30% methanol in DCM to afford (R)-5-bromo-2-(4-((1-methylpiperidin-3-yl)amino)pyrrolo[1,2-d][1,2,4]triazin-1-yl)phenol as a pale green solid (8 mg, 35% yield). MS m/z 402.2, 404.2 [M+H]⁺. ¹H NMR (400 MHz, MeOD) δ 7.96-7.81 (m, 2H), 7.27-6.96 (m, 4H), 4.47-4.38 (m, 1H), 3.28-3.13 (m, 1H), 2.90-2.76 (m, 1H), 2.55-2.34 (m, 5H), 2.17-2.02 (m, 1H), 1.87-1.52 (m, 3H). NH and OH peak not observed.

### Example 45

### Preparation of Compound I-490

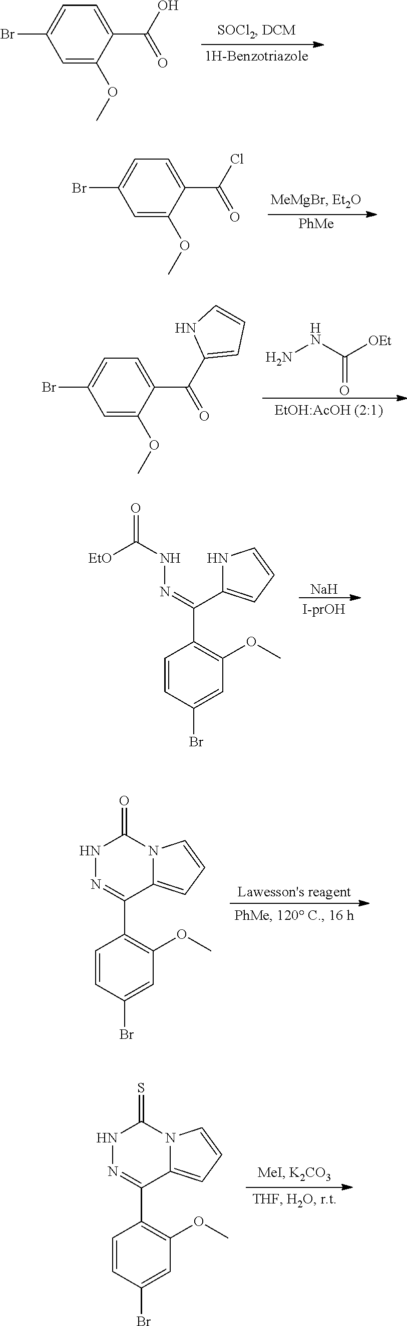

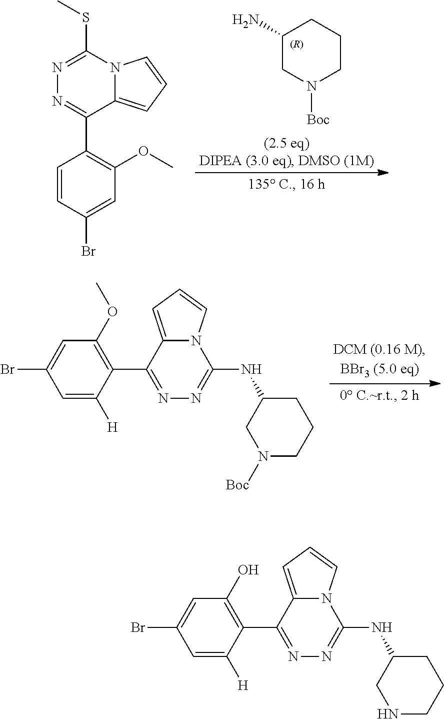

### Step 1. 4-Bromo-2-methoxybenzoyl chloride

To a solution of 4-bromo-2-methoxybenzoic acid (9.00 g, 40.0 mmol, 1.0 eq.) in DCM (80 mL) was added a mixture of 1H-benzotriazole (5.80 g, 48.0 mmol, 1.25 eq.) and SOCl₂ (3.5 mL, 48.7 mmol, 1.25 eq.) in DCM (20 mL) dropwise at 0° C. The reaction mixture was stirred at 0° C. for 20 min. The mixture was warmed to room temperature and filtered. The filtrate was dried over anhydrous Mg₂SO₄, concentrated in vacuo to give crude 4-bromo-2-methoxybenzoyl chloride (9.7 g) as yellow oil, which was used to the next step without further purification.

### Step 2. (4-Bromo-2-methoxyphenyl)(1H-pyrrol-2-yl)methanone

To a solution of CH₃MgBr (3M in THF, 12.9 mL, 38.7 mmol, 1.0 eq.) in Et₂O (30 mL) at 0° C. was added 1H-pyrrole (2.6 g, 38.9 mmol, 1.0 eq.) and the temperature was slowly increased to 40° C. The reaction mixture was stirred at 40° C. for 1 hour. After cooling to 0° C., 4-bromo-2-methoxybenzoyl chloride (9.70 g, 38.9 mmol, 1.0 eq) was added to the reaction mixture at 0° C. The reaction mixture was warmed to rt and stirred at 40° C. for another 1 hour. After Cooling to room temperature, the reaction mixture was quenched with aq. NH₄Cl and extracted with ethyl acetate (200 mL×3). The organic layer was washed with water, dried over magnesium sulfate and concentrated under reduced pressure. The concentrated residue was purified by silica gel column chromatography eluting with 0-30% EtOAc in hexanes to give 4-bromo-2-methoxyphenyl(1H-pyrrol-2-yl) methanone (5.30 g, 48% yield) as a brown solid. MS m/z 280.0, 282.0 [M+H]⁺.

### Step 3. Ethyl (Z)-2-((4-bromo-2-methoxyphenyl)(1H-pyrrol-2-yl)methylene)hydrazine-1-carboxylate

A mixture of (4-bromo-2-methoxyphenyl) (1H-pyrrol-2-yl)methanone (7.63 g, 27.2 mmol, 1.0 eq.), ethyl hydrazinecarboxylate (14.2 g, 136 mmol, 5.0 eq.) in AcOH (27 mL) and EtOH (54 mL) was heated at 100° C. for 16 h. Upon completion, the reaction mixture was cooled to room temperature and concentrated in vacuum. The residue was diluted with water (200 mL), neutralized with aqueous sat. NaHCO₃ to pH 8 and extracted with EtOAc (200 mL×3). The organic phase was dried over Na₂SO₄, filtered, and concentrated under reduced pressure. The crude product was purified by silica gel column chromatography eluting with 0-50% EtOAc in hexanes to afford ethyl (Z)-2-((4-bromo-2-methoxyphenyl) (1H-pyrrol-2-yl) methylene) hydrazine-1-carboxylate (7.1 g, 71% yield) as a purple solid. MS m/z 366.2, 368.0 [M+H]⁺.

### Step 4. 1-(4-Bromo-2-methoxyphenyl) pyrrolo[1,2-d][1,2,4] triazin-4(3H)-one

NaH (2.73 g, 68.3 mmol, 2.5 eq.) was added in portions to a stirred solution of ethyl (Z)-2-((4-bromo-2-methoxyphenyl) (1H-pyrrol-2-yl) methylene) hydrazine-1-carboxylate (10.0 g, 27.3 mmol, 1.0 eq.) in isopropyl alcohol (100 mL) at 0° C. Once the addition was completed, the mixture was heated to 80° C. and stirred at this temperature for 16 hours. Upon completion, the reaction mixture was cooled to room temperature. The mixture was quenched with water (150 mL) and extracted with EtOAc (150 mL×3). The organic phase was dried over Na₂SO₄, filtered and concentrated under reduced pressure. The crude product was treated with EtOAc (35 mL), filtered and washed with EtOAc. The solid was dried under vacuum to afford 1-(4-bromo-2-methoxyphenyl) pyrrolo[1,2-d][1,2,4] triazin-4(3H)-one (3.70 g, 11.6 mol, 42.5% yield) as a black solid. MS m/z 320.0, 322.2 [M+H]⁺.

### Step 5. 1-(4-Bromo-2-methoxyphenyl) pyrrolo[1,2-d][1,2,4] triazine-4(3H)-thione

A mixture of 1-(4-bromo-2-methoxyphenyl) pyrrolo[1,2-d][1,2,4] triazin-4(3H)-one (1.70 g, 5.31 mmol, 1.0 eq.) and Lawson's reagent (3.22 g, 7.96 mmol, 1.5 eq.) in toluene (53 mL) was heated at 120° C. for 16 h. Upon completion, the reaction mixture was cooled to room temperature and diluted with EtOAc (80 mL). The organic phase was washed with aqueous sat. NaHCO₃ (100 mL) and brine (100 mL), dried over Na₂SO₄ filtered, and concentrated under reduced pressure. The crude product was triturated with EtOAc (20 mL), filtered and washed with EtOAc. The solid was dried under vacuum to afford 1-(4-bromo-2-methoxyphenyl) pyrrole[1,2-d][1,2,4] triazin-4(3H)-one (1.10 g, 3.27 mol, 61.6% yield) as a brown solid. MS m/z 336.0, 337.9 [M+H]⁺.

### Step 6. 1-(4-Bromo-2-methoxyphenyl)-4-(methylthio) pyrrolo[1,2-d][1,2,4] triazine

To a mixture of 1-(4-bromo-2-methoxyphenyl) pyrrolo[1,2-d][1,2,4] triazin-4(3H)-one (1.68 g, 5.00 mmol, 1.0 eq.) and K₂CO₃ (1.73 g, 12.5 mmol, 2.5 eq.) in THE (12 mL) and water (6 mL) was added iodomethane (0.5 mL, 2.28 g/mL, 7.50 mmol, 1.5 eq.) dropwise at 0° C. The reaction was stirred at 0° C. for 1 hour. The reaction mixture was diluted with water (50 mL) and extracted with EtOAc (50 mL×3). The organic layer was dried over Na₂SO₄, filtered and concentrated under reduced pressure. The crude product was purified by silica gel column chromatography eluting with 0-30% EtOAc in hexanes to afford 1-(4-bromo-2-methoxyphenyl)-4-(methylthio) pyrrolo[1,2-d][1,2,4] triazine (1.40 g, 80% yield) as white solids. MS m/z 349.9, 352.1 [M+H]⁺.

### Step 7. tert-Butyl (R)-3-((1-(4-bromo-2-methoxyphenyl) pyrrolo[1,2-d][1,2,4]triazin-4-yl) amino) piperidine-1-carboxylate

A mixture of 1-(4-bromo-2-methoxyphenyl)-4-(methylthio)pyrrolo[1,2-d][1,2,4] triazine (2.00 g, 5.71 mmol, 1.0 eq.), tert-butyl (R)-3-aminopiperidine-1-carboxylate (2.86 g, 14.3 mmol, 2.5 eq.) and DIPEA (3.00 mL, 0.742 g/mL, 17.1 mmol, 3.0 eq.) in DMSO (4 mL) was heated at 135° C. for 16 h. Upon completion, the reaction mixture was cooled to room temperature and diluted with water (80 mL) and extracted with EtOAc (80 mL×3). The organic phase was washed with brine (100 mL), dried over Na₂SO₄, filtered and concentrated under reduced pressure. The crude product was purified by silica gel column chromatography eluting with 0-50% EtOAc in hexanes to afford tert-butyl (R)-3-((1-(4-bromo-2-methoxyphenyl) pyrrolo[1,2-d][1,2,4] triazin-4-yl) amino) piperidine-1-carboxylate (1.17 g, 41% yield) as brown solids. MS m/z 502.3, 504.4 [M+H]⁺.

### Step 8. (R)-5-Bromo-2-(4-(piperidin-3-ylamino) pyrrolo[1,2-d][1,2,4] triazin-1-yl) phenol

To a solution of tert-butyl (R)-3-((1-(4-bromo-2-methoxyphenyl) pyrrolo[1,2-d][1,2,4]triazin-4-yl) amino) piperidine-1-carboxylate (1.17 g, 2.33 mmol, 1.0 eq.) in DCM (10 mL) was added boron tribromide (12 mL, 1.0 M in DCM, 11.7 mmol, 5.0 eq.) dropwise at 0° C. Once the addition was completed, the mixture was warmed to room temperature and stirred for 1 hour. The reaction mixture was quenched with MeOH (50 mL) at 0° C. slowly and concentrated. The mixture was diluted with water (10 mL), neutralized with aqueous sat. NaHCO₃ to pH 8 and extracted with EtOAc (50 mL×3). The organic phase was dried over Na₂SO₄, filtered and concentrated under reduced pressure. The crude product was purified by silica gel column chromatography eluting with 0-10% MeOH in DCM to afford (R)-5-bromo-2-(4-(piperidin-3-ylamino) pyrrole[1,2-d][1,2,4] triazin-1-yl) phenol (492 mg, 55% yield) as a brown solid. MS m/z 388.1, 390.1 [M+H]⁺; (400 Hz, DMSO-d6) δ: 7.91 (d, J=8.4 Hz, 1H), 7.46-7.45 (m, 1H), 7.22 (d, J=3.6 Hz, 1H), 7.18 (d, J=2.0 Hz, 1H), 7.13 (dd, J=8.4 Hz, 2.0 Hz, 1H), 7.09-7.08 (m, 1H), 4.18 (br s, 1H), 3.27-3.23 (m, 2H), 2.91-2.88 (m, 1H), 2.56-2.53 (m, 1H), 2.09-2.07 (m, 1H), 1.75-1.71 (m, 1H), 1.65-1.50 (m, 2H).

The compounds below were prepared according to the procedure of Example 45 by substituting the appropriate starting materials, reagents, and reaction conditions.

| Compound | Spectral Data |
|---|---|
| I-504 | MS m/z 470.2, 473.2 [M + H]⁺; ¹H NMR (400 MHz, MeOD) δ 7.85 (d, J = 2.9 |
| | Hz, 1H), 7.72 (dd, J = 10.6, 2.6 Hz, 2H), 7.58 (d, J = 8.2 Hz, 1H), 7.03-6.91 |
| | (m, 1H), 6.56 (d, J = 3.6 Hz, 1H), 4.59-4.40 (m, 1H), 3.26-3.07 (m, 1H), |
| | 2.86-2.71 (m, 1H), 2.44-2.27 (m, 5H), 2.16-2.02 (m, 1H), 1.98-1.83 (m, |
| | 1H), 1.83-1.71 (m, 1H), 1.71-1.54 (m, 1H). NH peak not observed. |
| I-529 | MS m/z 432.3, 434.3 [M + H]⁺; ¹H NMR (400 MHz, CD₃OD) δ 7.94 (s, 1H), |
| | 7.88 (d, J = 8.3 Hz, 1H), 7.21 (d, J = 3.8 Hz, 1H), 7.20-7.12 (m, 2H), 7.06 (s, |
| | 1H), 4.42 (s, 1H), 3.74 (d, J = 6.2 Hz, 2H), 3.20 (d, J = 10.8 Hz, 1H), 2.79 (d, |
| | J = 11.1 Hz, 1H), 2.74-2.59 (m, 2H), 2.46 (s, 2H), 2.13-1.95 (m, 1H), 1.89 (d, |
| | J = 11.4 Hz, 1H), 1.85-1.63 (m, 2H). 2H not observed (NH and OH). |
| I-564 | MS m/z 416.0, 417.9 [M + H]⁺; ¹H NMR (400 MHz, METHANOL-d4) δ 8.43 (s, |
| | 1H, formic acid), 7.88 (s, 1H), 7.83 (d, J = 8.00 Hz, 1H), 7.22-7.12 (m, 3H), |
| | 7.08 (s, 1H), 4.59-4.48 (m, 1H), 3.86-3.73 (m, 1H), 3.47-3.38 (m, 1H), |
| | 3.20-3.10 (m, 2H), 3.01-2.81 (m, 2H), 2.31-2.21 (m, 1H), 2.29-2.10 (m, |
| | 1H), 2.01-1.74 (m, 2H), 1.35 (t, J = 6.9 Hz, 3H). 2H not observed (NH and OH). |

### Example 46

### Preparation of Compound I-329

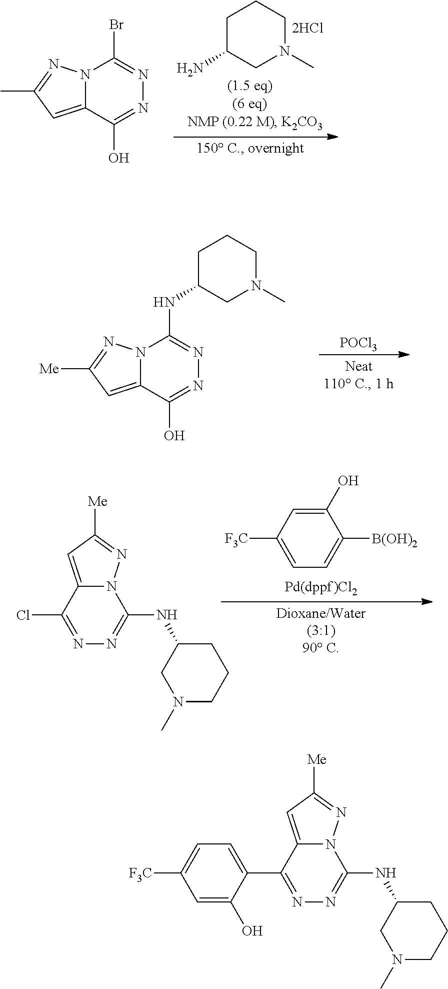

### Step 1. (R)-2-Methyl-7-((1-methylpiperidin-3-yl)amino)pyrazolo[1,5-d][1,2,4]triazin-4-ol

A mixture of 7-bromo-2-methylpyrazolo[1,5-d][1,2,4]triazin-4-ol (Intermediate 4a, 3.00 g, 13.2 mmol, 1.0 eq.), (R)-1-methylpiperidin-3-amine hydrochloride (3.69 g, 19.7 mmol, 1.5 eq.) and potassium carbonate (10.9 g, 79.2 mmol, 6.0 eq.) in NMP (60 mL) was heated at 150° C. for 16 h. After cooling to rt, the reaction mixture was filtered and the filtrate was directly purified by reversed-phase column chromatography eluting with 0-100% ACN in H₂O (0.5% NH₄OH) to afford (R)-2-methyl-7-((1-methylpiperidin-3-yl)amino)pyrazolo[1,5-d][1,2,4]triazin-4-ol (1.80 g, 52% yield). MS m/z 263.2 [M+H]⁺; ¹H NMR (400 MHz, DMSO-d6) δ 11.55 (s, 1H), 6.91 (s, 1H), 6.49 (d, J=8.2 Hz, 1H), 3.85-3.72 (m, 1H), 2.74-2.65 (m, 1H), 2.45-2.42 (m, 1H), 2.41 (s, 3H), 2.17 (s, 3H), 2.15-2.05 (m, 2H), 1.76-1.61 (m, 2H), 1.60-1.44 (m, 2H).

### Step 2. (R)-4-Chloro-2-methyl-N-(1-methylpiperidin-3-yl)pyrazolo[1,5-d][1,2,4]triazin-7-amine

(R)-2-Methyl-7-((1-methylpiperidin-3-yl)amino)pyrazolo[1,5-d][1,2,4]triazin-4-ol (500.0 mg, 1.906 mmol) was treated with POCl₃ (590.4 mg, 3.59 mL, 38.12 mmol). The mixture was then heated at 110° C. for 1 hour, upon which UPLC/MS showed complete conversion to product. The POCl₃ was then evaporated under reduced pressure, and the resulting residue was neutralized with saturated NaHCO₃ solution until pH >7. The aqueous layer was then extracted with CH₂Cl₂ (5×). The organic layer collected was washed with water and brine, dried over Na₂SO₄, and evaporated to dryness. The resulting crude (R)-4-chloro-2-methyl-N-(1-methylpiperidin-3-yl)pyrazolo[1,5-d][1,2,4]triazin-7-amine (492.5 mg, 92% yield) was obtained as brown solid and was used without further purification. MS m/z 281.3, 283.3 [M+H]⁺.

Step 3. ((R)-2-(2-methyl-7-((1-methylpiperidin-3-yl)amino)pyrazolo[1,5-d][1,2,4]triazin-4-yl)-5-(trifluoromethyl)phenol A mixture of (R)-4-chloro-2-methyl-N-(1-methylpiperidin-3-yl)pyrazolo[1,5-d][1,2,4]triazin-7-amine (50.0 mg, 0.178 mmol), [2-hydroxy-4-(trifluoromethyl)phenyl]boronic acid (44.0 mg, 0.214 mmol), Pd(dppf)Cl₂ CH₂Cl₂ (14.5 mg, 0.0178 mmol), and K₂CO₃ (73.8 mg, 0.534 mmol) was dissolved in dioxane (2.1 mL) and water (0.7 mL). The reaction mixture was then purged and bubbled with nitrogen gas for 5 minutes, followed by stirring and heating at 90° C. for 3 hours. The solvent was evaporated, and the resulting residue was partitioned with water and CH₂Cl₂. The organic layer was collected, dried with Na₂SO₄, and evaporated. The crude product was then purified by flash column chromatography, eluting with 0-10% MeOH in DCM. The compound was purified again with elution of 100% EtOAc for 5 CV followed by gradient elution of CH₂Cl₂:MeOH=100:0 to 90:10 over 15 CV. (R)-2-(2-methyl-7-((1-methylpiperidin-3-yl)amino)pyrazolo[1,5-d][1,2,4]triazin-4-yl)-5-(trifluoromethyl)phenol (24.4 mg, 33.7% yield) was obtained as brown solid. MS m/z 407.5 [M+H]⁺; ¹H NMR (400 MHz, CD₃OD) δ 8.11 (d, J=8.2 Hz, 1H), 7.29 (d, J=8.4 Hz, 1H), 7.26 (s, 1H), 7.13 (s, 1H), 4.52-4.39 (m, 1H), 3.11-2.98 (m, 1H), 2.78-2.63 (m, 1H), 2.59 (s, 3H), 2.52-2.26 (m, 5H), 2.11-1.96 (m, 1H), 1.96-1.83 (m, 1H), 1.83-1.63 (m, 2H). 1 OH and 1 NH signals are not observed.

### Example 47

### Preparation of Compound I-374, I-396, I-418, and I-419

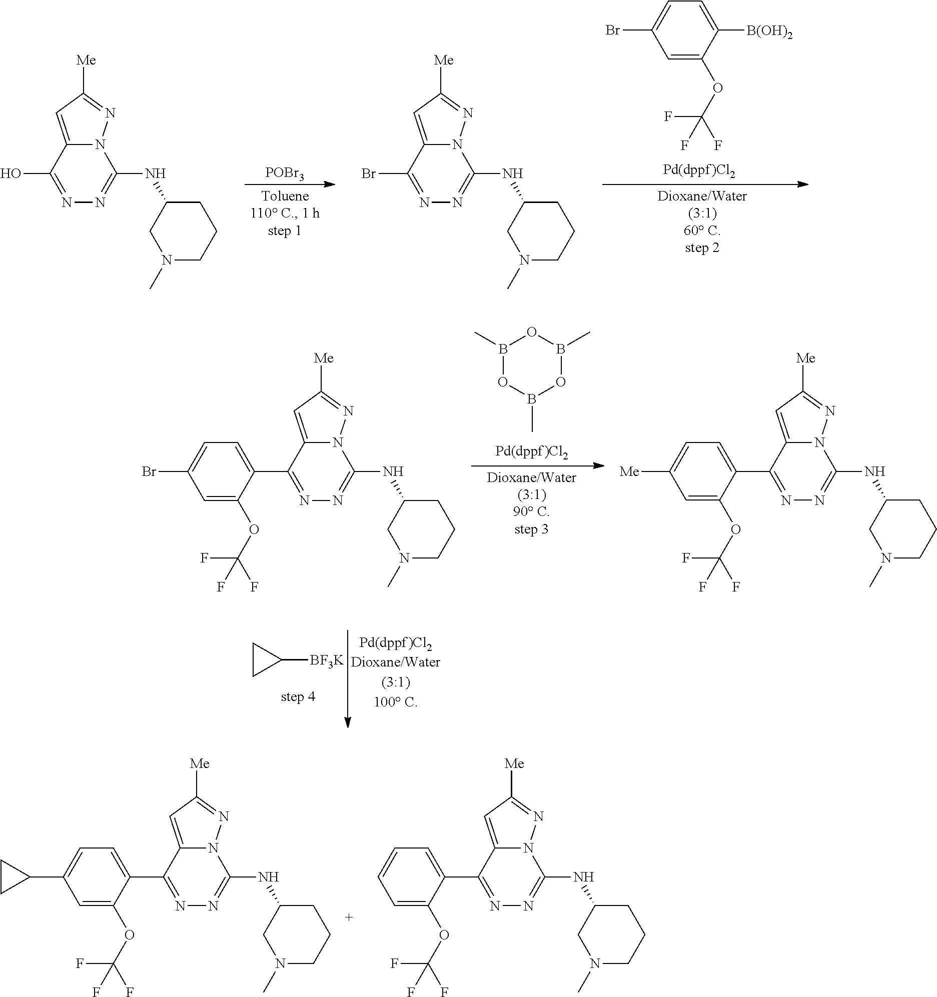

### Step 1. (R)-4-Bromo-2-methyl-N-(1-methylpiperidin-3-yl)pyrazolo[1,5-d][1,2,4]triazin-7-amine

(R)-2-Methyl-7-((1-methylpiperidin-3-yl)amino)pyrazolo[1,5-d][1,2,4]triazin-4-ol (prepared as Example 46, step 1, 300 mg, 1.14 mmol) and POBr₃ (1.73 g, 5.72 mmol) were suspended in dry toluene (2.0 mL). The mixture was stirred vigorously and heated to 110° C. for 1 hour. Upon consumption of all starting material, the reaction was carefully quenched with saturated NaHCO₃ solution until pH >7. The aqueous solution was then extracted with CH₂Cl₂ (5×). The combined organic layer was washed with water and brine, dried over Na₂SO₄, and evaporated to dryness. The resulting crude (R)-4-bromo-2-methyl-N-(1-methylpiperidin-3-yl)pyrazolo[1,5-d][1,2,4]triazin-7-amine (310.1 mg, 83% yield) was used in the next step without further purification. MS m/z 325.3, 327.3 [M+H]⁺.

### Step 2. (R)-4-(4-Bromo-2-(trifluoromethoxy)phenyl)-2-methyl-N-(1-methylpiperidin-3-yl)pyrazolo[1,5-d][1,2,4]triazin-7-amine

A mixture of (R)-4-bromo-2-methyl-N-(1-methylpiperidin-3-yl)pyrazolo[1,5-d][1,2,4]triazin-7-amine (296.9 mg, 0.9128 mmol), (4-bromo-2-(trifluoromethoxy)phenyl)boronic acid (200.0 mg, 0.7022 mmol), Pd(dppf)Cl₂ CH₂Cl₂ (28.7 mg, 0.035 mmol), and K₂CO₃ (291 mg, 2.11 mmol) was dissolved in dioxane (2.1 mL) and water (0.70 mL). The reaction mixture was then purged and bubbled with nitrogen gas for 5 minutes, followed by stirring and heating at 60° C. for 5 hours. The solvent was evaporated, and the resulting residue was partitioned with water and CH₂Cl₂. The organic layer was collected, dried with Na₂SO₄, and evaporated. The crude product was then purified with C18 reverse phase column (gradient elution of MeCN (0.1% formic acid):H₂O (0.1% formic acid)=5:95 to 100:0 over 20 CV) to obtain (R)-4-(4-bromo-2-(trifluoromethoxy)phenyl)-2-methyl-N-(1-methylpiperidin-3-yl)pyrazolo[1,5-d][1,2,4]triazin-7-amine formic acid salt (96 mg, 22% yield) as white powder. (Small portion (15.0 mg) of the salt was neutralized with NaHCO₃ (aq.) to free base for in vitro studies). MS m/z 485.3, 487.3 [M+H]⁺; ¹H NMR (400 MHz, CD₃OD) δ 7.83-7.69 (m, 2H), 7.64 (d, J=8.2 Hz, 1H), 6.51 (s, 1H), 4.60-4.47 (m, 1H), 3.10-2.92 (m, 1H), 2.91-2.27 (m, 8H), 2.22-2.05 (m, 1H), 2.05-1.92 (m, 1H), 1.92-1.67 (m, 2H). 1 CH signal overlaps with solvent peak. 1 NH signal is not observed.

### Step 3. (R)-2-Methyl-4-(4-methyl-2-(trifluoromethoxy)phenyl)-N-(1-methylpiperidin-3-yl)pyrazolo[1,5-d][1,2,4]triazin-7-amine

A mixture of (R)-4-(4-bromo-2-(trifluoromethoxy)phenyl)-2-methyl-N-(1-methylpiperidin-3-yl)pyrazolo[1,5-d][1,2,4]triazin-7-amine formic acid salt (34.0 mg, 0.0701 mmol), trimethylboroxine (26.4 mg, 0.210 mmol), Pd(dppf)Cl₂ CH₂Cl₂ (5.72 mg, 0.00701 mmol), and K₂CO₃ (290.0 mg, 0.210 mmol) was dissolve in dioxane (2.1 mL) and water (0.7 mL). The reaction mixture was then purged and bubbled with nitrogen gas for 5 minutes, followed by stirring and heating at 90° C. for 16 hours. The solvent was then evaporated under reduced pressure, and the resulting residue was partitioned with water and CH₂Cl₂. The organic layer was collected, dried with Na₂SO₄, and evaporated to dryness. The crude product was then purified by flash column chromatography (gradient elution of CH₂Cl₂:MeOH=100:0 to 90:10 over 20 CV) to obtain (R)-2-methyl-4-(4-methyl-2-(trifluoromethoxy)phenyl)-N-(1-methylpiperidin-3-yl)pyrazolo[1,5-d][1,2,4]triazin-7-amine (21.9 mg, 74% yield) as pale brown solid. MS m/z 421.4 [M+H]⁺; ¹H NMR (500 MHz, CD₃OD) δ 7.56 (d, J=7.8 Hz, 1H), 7.37 (d, J=7.9 Hz, 1H), 7.34 (s, 1H), 6.45 (s, 1H), 4.51-4.43 (m, 1H), 3.05 (s, 1H), 2.83-2.13 (m, 11H), 2.12-1.57 (m, 5H). 1 NH signal is not observed.

### Step 4. (R)-4-(4-Cyclopropyl-2-(trifluoromethoxy)phenyl)-2-methyl-N-(1-methylpiperidin-3-yl)pyrazolo[1,5-d][1,2,4]triazin-7-amine and (R)-2-methyl-N-(1-methylpiperidin-3-yl)-4-(2-(trifluoromethoxy)phenyl)pyrazolo[1,5-d][1,2,4]triazin-7-amine

A mixture of (R)-4-(4-bromo-2-(trifluoromethoxy)phenyl)-2-methyl-N-(1-methylpiperidin-3-yl)pyrazolo[1,5-d][1,2,4]triazin-7-amine formic acid salt (30.0 mg, 0.0618 mmol), potassium cyclopropyltrifluoroborate (91.5 mg, 0.618 mmol), Pd(dppf)Cl₂ CH₂Cl₂ (10.1 mg, 0.01236 mmol), and K₂CO₃ (85.4 mg, 0.618 mmol) was dissolved in dioxane (2.1 mL) and water (0.7 mL). The reaction mixture was then purged and bubbled with nitrogen gas for 5 minutes, followed by stirring and heating at 100° C. for 16 hours. The solvent was then evaporated under reduced pressure, and the resulting residue was partitioned with water and CH₂Cl₂. The organic layer was collected, dried with Na₂SO₄, and evaporated to dryness. The crude product was then purified with C18 reverse phase column (gradient elution of MeCN (0.1% formic acid):H₂O (0.1% formic acid)=5:95 to 100:0 over 20 CV) to obtain (R)-4-(4-cyclopropyl-2-(trifluoromethoxy)phenyl)-2-methyl-N-(1-methylpiperidin-3-yl)pyrazolo[1,5-d][1,2,4]triazin-7-amine formic acid salt (5.6 mg, 20% yield) as white powder and (R)-2-methyl-N-(1-methylpiperidin-3-yl)-4-(2-(trifluoromethoxy)phenyl)pyrazolo[1,5-d][1,2,4]triazin-7-amine (3.1 mg, 12%) as minor product, respectively.

Compound I-419: MS m/z 447 [M+H]⁺; ¹H NMR (500 MHz, CD₃OD) δ 8.50 (s, 1H, formate CH), 7.51 (d, J=7.9 Hz, 1H), 7.19 (d, J=8.0 Hz, 1H), 7.16 (s, 1H), 6.42 (s, 1H), 4.53-4.35 (m, 1H), 3.23-3.03 (m, 1H), 2.89-2.70 (m, 1H), 2.70-2.14 (m, 8H), 2.14-1.96 (m, 2H), 1.96-1.82 (m, 1H), 1.83-1.59 (m, 2H), 1.22-0.93 (m, 2H), 0.93-0.62 (m, 2H). 1 NH signals not observed.

Compound I-420: MS m/z 407 [M+H]⁺; ¹H NMR (500 MHz, CD₃OD) δ 8.52 (s, 1H, formate CH), 7.83-7.62 (m, 2H), 7.62-7.41 (m, 2H), 6.48 (s, 1H), 4.61-4.47 (m, 1H), 3.16-2.80 (m, 2H), 2.80-2.27 (m, 7H), 2.17-2.04 (m, 1H), 2.04-1.90 (m, 1H), 1.90-1.67 (m, 2H). 1 NH signal not observed. 1 CH signal overlaps with solvent peak.

### Example 48

### Preparation of Compound I-375

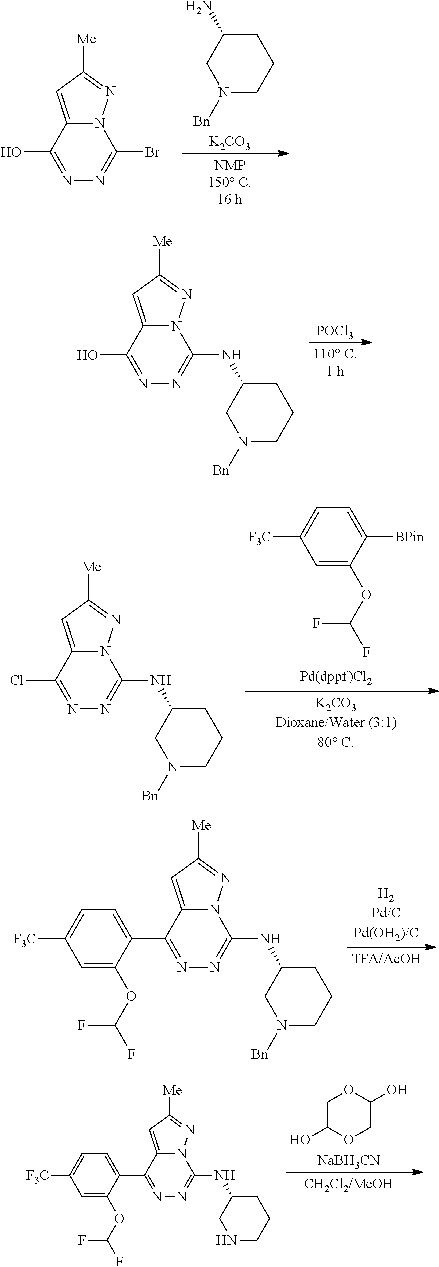

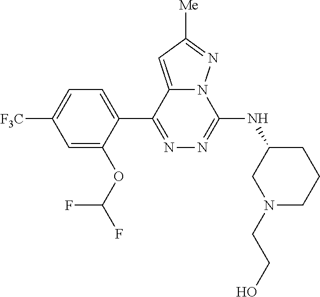 [SMILES: Cc1cc2c(-c3ccc(C(F)(F)F)cc3OC(F)F)nnc(N[C@@H]3CCCN(CCO)C3)n2n1]

### Step 1. (R)-7-((1-Benzylpiperidin-3-yl)amino)-2-methylpyrazolo[1,5-d][1,2,4]triazin-4-ol

A mixture of 7-bromo-2-methylpyrazolo[1,5-d][1,2,4]triazin-4-ol (200 mg, 0.87 mmol) and (R)-1-benzylpiperidin-3-amine (831 mg, 4.37 mmol) in dry NMP (4.5 mL) was stirred under nitrogen atmosphere at 150° C. for 16 hours. After all starting material has been consumed, the solution was diluted with EtOAc. It was then washed with saturated NaHCO₃ solution (3×), water (3×), and brine, dried over Na₂SO₄, and evaporated under reduced pressure. The resulting residue was then purified by flash column chromatography (gradient elution of CH₂Cl₂: MeOH=100:0 to 90:10 over 20 CV). (R)-7-((1-benzylpiperidin-3-yl)amino)-2-methylpyrazolo[1,5-d][1,2,4]triazin-4-ol (298 mg, >99% yield) was obtained as yellow foam. MS m/z 339.4 [M+H]⁺.

### Step 2. (R)—N-(1-Benzylpiperidin-3-yl)-4-chloro-2-methylpyrazolo[1,5-d][1,2,4]triazin-7-amine

(R)-7-((1-Benzylpiperidin-3-yl)amino)-2-methylpyrazolo[1,5-d][1,2,4]triazin-4-ol (200.0 mg, 0.5910 mmol) was treated with POCl₃ (1.81 g, 1.10 mL, 11.8 mmol). The reaction was stirred and heated at 110° C. for 1 hour. After consumption of the starting material, the POCl₃ was evaporated under reduced pressure, and the resulting residue was neutralized with saturated NaHCO₃ solution until pH >7. The aqueous layer was then extracted with CH₂Cl₂ (5×). The organic layer collected was washed with water and brine, dried over Na₂SO₄, and evaporated to dryness. The resulting crude (R)—N-(1-benzylpiperidin-3-yl)-4-chloro-2-methylpyrazolo[1,5-d][1,2,4]triazin-7-amine (203 mg, 96% yield) was obtained as brown solid and used in the next step without further purification. MS m/z 357.4, 359.4 [M+H]⁺.

### Step 3. (R)—N-(1-Benzylpiperidin-3-yl)-4-(2-(difluoromethoxy)-4-(trifluoromethyl)phenyl)-2-methylpyrazolo[1,5-d][1,2,4]triazin-7-amine

A mixture of (R)—N-(1-benzylpiperidin-3-yl)-4-chloro-2-methylpyrazolo[1,5-d][1,2,4]triazin-7-amine (150 mg, 0.42 mmol), 2-(2-(difluoromethoxy)-4-(trifluoromethyl)phenyl)-4,4,5,5-tetramethyl-1,3,2-dioxaborolane (171 mg, 0.50 mmol), Pd(dppf)Cl₂ CH₂Cl₂ (34 mg, 0.042 mmol), and K₂CO₃ (174 mg, 1.26 mmol) was dissolved in dioxane (3.0 mL) and water (1.0 mL). The reaction mixture was then purged and bubbled with nitrogen gas for 5 minutes, followed by stirring and heating at 80° C. for 1 hours. The solvent was evaporated, and the resulting residue was partitioned with water and CH₂Cl₂. The organic layer was collected, dried with Na₂SO₄, and evaporated. The crude product was then purified by flash column chromatography (gradient elution of CH₂Cl₂:MeOH=100:0 to 90:10 over 20 CV). (R)—N-(1-benzylpiperidin-3-yl)-4-(2-(difluoromethoxy)-4-(trifluoromethyl)phenyl)-2-methylpyrazolo[1,5-d][1,2,4]triazin-7-amine was obtained along with inseparable impurities (total 248 mg), which was used in the next step without further purification. MS m/z 533.4 [M+H]⁺.

### Step 4. (R)—N-(1-Benzylpiperidin-3-yl)-4-(2-(difluoromethoxy)-4-(trifluoromethyl)phenyl)-2-methylpyrazolo[1,5-d][1,2,4]triazin-7-amine

Crude (R)—N-(1-benzylpiperidin-3-yl)-4-(2-(difluoromethoxy)-4-(trifluoromethyl)phenyl)-2-methylpyrazolo[1,5-d][1,2,4]triazin-7-amine (50.0 mg, 0.282 mmol) in THE (2.0 mL) and acetic acid (1.0 mL) was treated with wet Pd/C (10% wt, 5.0 mg) and Pd(OH₂)/C (20% wt, 5.0 mg). The flask was then purged with hydrogen and the reaction was stirred for 16 hours. After all starting material has been consumed, the mixture was filtered over a pad of celite. The filtrate was then treated saturated NaHCO₃ solution until pH >7. The aqueous layer was extracted with CH₂Cl₂ (5×). The combined organic layer was then washed with water and brine, dried over Na₂SO₄, and evaporated to dryness. The crude product was purified by flash column chromatography (gradient elution of CH₂Cl₂:MeOH=90:10 to 65:35) to obtain (R)-4-(2-(difluoromethoxy)-4-(trifluoromethyl)phenyl)-2-methyl-N-(piperidin-3-yl)pyrazolo[1,5-d][1,2,4]triazin-7-amine (33.4 mg, 80% yield) as brown solid. MS m/z 443.4 [M+H]⁺.

### Step 5. (R)-2-(3-((4-(2-(Difluoromethoxy)-4-(trifluoromethyl)phenyl)-2-methylpyrazolo[1,5-d][1,2,4]triazin-7-yl)amino)piperidin-1-yl)ethan-1-ol

(R)-4-(2-(Difluoromethoxy)-4-(trifluoromethyl)phenyl)-2-methyl-N-(piperidin-3-yl)pyrazolo[1,5-d][1,2,4]triazin-7-amine (33.4 mg, 0.0755 mmol) and 1,4-dioxane-2,5-diol (6.80 mg, 0.0566 mmol) were dissolved in CH₂Cl₂ (1.0 mL) and MeOH (1.0 mL). Into the solution was then added sodium cyanoborohydride (5.69 mg, 0.0906 mmol). The solution was stirred for 10 minutes, upon which complete formation to product was observed by UPLC/MS. The reaction was then quenched with addition of saturated NaHCO₃ solution dropwise. The solution was partitioned with CH₂Cl₂ and water. The organic layer was collected, dried over Na₂SO₄, and evaporated to dryness. The crude product was purified with flash column chromatography (gradient elution of CH₂Cl₂:MeOH=100:0 to 80:20) to obtain (R)-2-(3-((4-(2-(difluoromethoxy)-4-(trifluoromethyl)phenyl)-2-methylpyrazolo[1,5-d][1,2,4]triazin-7-yl)amino)piperidin-1-yl)ethan-1-ol (22.4 mg, 61%) as white solid. MS m/z 487.4 [M+H]⁺; (400 MHz, CD3OD) δ 7.85 (d, J=8.1 Hz, 1H), 7.74 (d, J=8.3 Hz, 1H), 7.67 (s, 1H), 6.94 (t, J=73.0 Hz, 1H), 6.49 (s, 1H), 4.54-4.43 (m, 1H), 3.79-3.66 (m, 2H), 3.11-2.98 (m, 1H), 2.87-2.29 (m, 8H), 2.07-1.93 (m, 1H), 1.92-1.63 (m, 3H). 1 OH and 1 NH signals are not observed.

The compounds below were prepared according to the procedure of Example 48 by substituting the appropriate starting materials, reagents and reaction conditions.

| Compound | Spectral Data |
|---|---|
| I-327 | MS m/z 377.4 [M − H]−; ¹H NMR (400 MHz, METHANOL-d4) δ 7.76 (d, J = |
| | 8.1 Hz, 1H), 7.03 (s, 1H), 6.73-6.64 (m, 2H), 4.37 (td, J = 4.1, 7.9 Hz, 1H), |
| | 3.06-2.92 (m, 1H), 2.68-2.53 (m, 4H), 2.44-2.26 (m, 5H), 2.07-1.95 (m, 1H), |
| | 1.95-1.81 (m, 2H), 1.81-1.60 (m, 2H), 1.08-0.96 (m, 2H), 0.79-0.69 (m, |
| | 2H); 2H (OH and NH) wasn't observed |
| I-328 | MS m/z 395.2 [M + H]⁺; ¹H NMR (400 MHz, CD₃OD) δ 7.88 (d, J = 8.8 Hz, |
| | 1H), 7.12 (s, 1H), 6.60 (dd, J = 8.9, 2.5 Hz, 1H), 6.54 (d, J = 2.5 Hz, 1H), |
| | 4.44-4.31 (m, 1H), 3.84 (s, 3H), 3.09-2.91 (m, 1H), 2.68-2.59 (m, 1H), 2.57 (s, |
| | 3H), 2.48-2.22 (m, 5H), 2.09-1.93 (m, 1H), 1.93-1.80 (m, 1H), 1.80-1.60 |
| | (m, 2H). 1 OH and 1 NH signals are not observed. |
| I-330 | MS m/z 457.4 [M + H]⁺; ¹H NMR (400 MHz, CD₃OD) δ 7.84 (d, J = 8.1 Hz, |
| | 1H), 7.74 (d, J = 8.1 Hz, 1H), 7.66 (s, 1H), 6.93 (t, J = 73.0 Hz, 1H), 6.49 (s, |
| | 1H), 4.53-4.42 (m, 1H), 3.09-2.95 (m, 1H), 2.74-2.57 (m, 1H), 2.53 (s, |
| | 3H), 2.46-2.22 (m, 5H), 2.12-1.95 (m, 1H), 1.95-1.81 (m, 1H), 1.81-1.60 |
| | (m, 2H). 1 CH signal overlaps with solvent peak. 1 NH signal not observed. |
| I-334 | MS m/z 416.5, 418.5 [M + H]⁺; ¹H NMR (400 MHz, METHANOL-d4) δ 7.78 |
| | (d, J = 8.3 Hz, 1H), 7.17-7.08 (m, 2H), 7.08-7.03 (m, 1H), 4.45-4.36 (m, 1H), |
| | 3.10-2.94 (m, 1H), 2.71-2.61 (m, 1H), 2.61-2.54 (m, 3H), 2.46-2.28 (m, 5H), |
| | 2.09-1.96 (m, 1H), 1.93-1.81 (m, 1H), 1.81-1.63 (m, 2H); 2H (OH and NH) |
| | wasn't observed |
| I-345 | MS m/z 433.5 [M + H]⁺; ¹H NMR (400 MHz, CD₃OD) δ 8.10 (d, J = 8.2 Hz, |
| | 1H), 7.29 (d, J = 8.4 Hz, 1H), 7.25 (s, 1H), 7.04 (s, 1H), 4.53-4.40 (m, 1H), |
| | 3.12-3.02 (m, 1H), 2.79-2.67 (m, 1H), 2.59-2.31 (m, 5H), 2.31-2.22 (m, |
| | 1H), 2.08-1.96 (m, 1H), 1.96-1.83 (m, 1H), 1.83-1.67 (m, 2H), 1.22-1.11 |
| | (m, 2H), 1.11-1.01 (m, 2H). 1 OH and 1 NH signals are not observed. |
| I-346 | MS m/z 483.4 [M + H]⁺; ¹H NMR (400 MHz, CD₃OD) δ 7.83 (d, J = 8.0 Hz, |
| | 1H), 7.73 (d, J = 8.1 Hz, 1H), 7.66 (s, 1H), 6.95 (t, J = 72.9 Hz, 1H), 6.38 |
| | (s, 1H), 4.56-4.39 (m, 1H), 3.08-2.94 (m, 1H), 2.71-2.56 (m, 1H), 2.53-2.24 |
| | (m, 5H), 2.24-2.14 (m, 1H), 2.09-1.95 (m, 1H), 1.94-1.81 (m, 1H), 1.81-1.61 |
| | (m, 2H), 1.15-1.03 (m, 2H), 1.01-0.87 (m, 2H). 1 NH signal is not observed. |
| I-347 | MS m/z 395.5 [M + H]⁺; ¹H NMR (400 MHz, CD₃OD) δ 7.87 (d, J = 8.8 Hz, |
| | 1H), 7.01 (s, 1H), 6.60 (dd, J = 8.8, 2.5 Hz, 1H), 6.53 (d, J = 2.5 Hz, 1H), |
| | 4.45-4.30 (m, 1H), 3.83 (s, 3H), 3.09-2.89 (m, 1H), 2.73-2.56 (m, 1H), |
| | 2.52-2.29 (m, 5H), 2.29-2.18 (m, 1H), 2.07-1.93 (m, 1H), 1.93-1.80 (m, 1H), |
| | 1.80-1.63 (m, 2H), 1.19-1.09 (m, 2H), 1.09-1.00 (m, 2H). 1 OH and 1 NH |
| | signals are not observed. |
| I-359 | MS m/z 371.4 [M + H]⁺; ¹H NMR (400 MHz, CD₃OD) δ 6.63 (s, 1H), 6.59 (d, J = |
| | 10.7 Hz, 1H), 6.41 (s, 1H), 4.53-4.37 (m, 1H), 3.11-2.94 (m, 1H), 2.72-2.57 |
| | (m, 1H), 2.51 (s, 3H), 2.45-2.16 (m, 8H), 2.08-1.95 (m, 1H), 1.95-1.80 |
| | (m, 1H), 1.80-1.63 (m, 2H). 1 OH and 1 NH signals are not observed. |
| I-363 | MS m/z 425.4 [M + H]⁺; ¹H NMR (400 MHz, CD₃OD) δ 7.16-6.96 (m, 2H), |
| | 6.44 (s, 1H), 4.59-4.37 (m, 1H), 3.17-2.97 (m, 1H), 2.79-2.62 (m, 1H), |
| | 2.59-2.31 (m, 8H), 2.15-1.96 (m, 1H), 1.96-1.82 (m, 1H), 1.82-1.62 (m, |
| | 2H). 1 OH and 1 NH signals are not observed. |
| I-381 | MS m/z 421.4 [M + H]⁺; ¹H NMR (500 MHz, CD₃OD) δ 7.52 (d, J = 8.5 Hz, |
| | 1H), 7.38 (d, J = 2.6 Hz, 1H), 7.32 (dd, J = 8.5, 2.6 Hz, 1H), 6.30 (s, 1H), |
| | 4.54-4.38 (m, 1H), 3.94 (s, 3H), 3.19-2.90 (m, 2H), 2.82-2.15 (m, 7H), |
| | 2.15-1.55 (m, 5H). 1 NH signal is not observed. |
| I-382 | MS m/z 459.4 [M + H]⁺; ¹H NMR (500 MHz, CD₃OD) δ 8.20 (s, 1H), 8.13 (d, J = |
| | 8.3 Hz, 1H), 7.87 (d, J = 8.0 Hz, 1H), 6.37 (s, 1H), 4.55-4.42 (m, 1H), |
| | 3.14-2.98 (m, 1H), 2.76-2.61 (m, 1H), 2.51 (s, 3H), 2.44-2.24 (m, 4H), |
| | 2.13-1.62 (m, 5H). 1 NH signal is not observed. |
| I-429 | MS m/z 397.4 [M + H]⁺; ¹H NMR (400 MHz, CD₃OD) δ 6.53 (s, 1H), 6.47 (d, J = |
| | 11.1 Hz, 1H), 6.43 (s, 1H), 4.54-4.38 (m, 1H), 3.13-2.97 (m, 1H), 2.78-2.61 |
| | (m, 1H), 2.51 (s, 3H), 2.46-2.29 (m, 5H), 2.11-1.98 (m, 1H), 1.96-1.86 |
| | (m, 2H), 1.79-1.66 (m, 2H), 1.08-0.99 (m, 2H), 0.80-0.69 (m, 2H). 1 |
| | OH and 1 NH signals are not observed. |
| I-455 | MS m/z 391.5, 393.5 [M + H]⁺; ¹H NMR (400 MHz, CD₃OD) δ 8.52 (s, 1H, |
| | formate CH), 6.96-6.74 (m, 2H), 6.44 (s, 1H), 4.61-4.44 (m, 1H), 3.27 (m, |
| | 1H, overlapped with CD₃OD peak), 3.08-2.76 (m, 2H), 2.76-2.30 (m, 7H), |
| | 2.20-2.05 (m, 1H), 2.05-1.91 (m, 1H), 1.91-1.68 (m, 2H). OH and NH not |
| | observed. |
| I-515 | MS m/z 385.6 [M + H]⁺; ¹H NMR (400 MHz, MeOD) δ 6.63 (s, 1H), 6.59 (d, J = |
| | 11.6 Hz, 1H), 6.42 (s, 1H), 4.53-4.40 (m, 1H), 3.20-3.08 (m, 1H), 2.83-2.69 |
| | (m, 1H), 2.56 (q, J = 7.6 Hz, 2H), 2.51 (s, 3H), 2.48-2.28 (m, 5H), 2.10-1.98 |
| | (m, 1H), 1.94-1.83 (m, 1H), 1.83-1.65 (m, 2H), 1.16 (t, J = 7.3 Hz, 3H). |
| | 1 OH and 1 NH signals are not observed. |
| I-550 | MS m/z 397.4 [M + H]⁺; ¹H NMR (400 MHz, MeOD) δ 7.76 (d, J = 7.9 Hz, |
| | 1H), 7.04 (s, 1H), 6.79 (s, 2H), 4.44-4.32 (m, 1H), 3.20-3.00 (m, 1H), |
| | 2.79-2.67 (m, 1H), 2.61-2.52 (m, 3H), 2.53-2.37 (m, 5H), 2.33 (s, 3H), |
| | 2.09-1.95 (m, 1H), 1.95-1.82 (m, 1H), 1.82-1.62 (m, 2H). NH and OH peak not |
| | observed. |
| I-551 | MS m/z 387.4 [M + H]⁺; ¹H NMR (400 MHz, MeOD) δ 6.63 (s, 1H), 6.59 (d, J = |
| | 11.6 Hz, 1H), 6.42 (s, 1H), 4.53-4.40 (m, 1H), 3.20-3.08 (m, 1H), 2.83-2.69 |
| | (m, 1H), 2.56 (q, J = 7.6 Hz, 2H), 2.51 (s, 3H), 2.48-2.28 (m, 5H), 2.10-1.98 |
| | (m, 1H), 1.94-1.83 (m, 1H), 1.83-1.65 (m, 2H), 1.16 (t, J = 7.3 Hz, 3H). |
| | NH and OH peak not observed. |
| I-554 | MS m/z 411.6 [M + H]⁺; ¹H NMR (400 MHz, MeOD) δ 6.53-6.59 (m, 1H), 6.43-6.53 |
| | (m, 2H), 4.53 (br s, 1H), 3.36-3.50 (m, 1H), 2.94-3.17 (m, 1H), 2.83 (br s, |
| | 2H), 2.58-2.76 (m, 2H), 2.53 (br s, 3H), 2.14 (br s, 1H), 1.88-2.05 (m, 2H), |
| | 1.70-1.88 (m, 2H), 1.25 (br t, J = 6.6 Hz, 3H), 1.05 (br d, J = 6.8 Hz, 2H), |
| | 0.76 (br s, 2H). OH and NH not observed. |
| I-555 | MS m/z 401.6 [M + H]⁺; ¹H NMR (400 MHz, MeOD) δ 6.45 (s, 1H), 6.42-6.28 |
| | (m, 2H), 4.56-4.40 (m, 1H), 3.82 (s, 3H), 3.22-3.10 (m, 1H), 2.90-2.72 (m, |
| | 1H), 2.70-2.56 (m, 2H), 2.56-2.35 (m, 5H), 2.11-2.02 (m, 1H), 1.95-1.85 |
| | (m, 1H), 1.80-1.69 (m, 2H), 1.17 (t, J = 7.0 Hz, 3H). 1 OH and 1 NH signals |
| | are not observed. |
| I-556 | MS m/z 471.6 [M + H]⁺; ¹H NMR (400 MHz, MeOD) δ 7.84 (d, J = 8.0 Hz, 1H), |
| | 7.74 (d, J = 8.1 Hz, 1H), 7.67 (s, 1H), 6.93 (t, J = 73.0 Hz, 1H), 6.49 (s, 1H), |
| | 4.54-4.43 (m, 1H), 3.17-3.00 (m, 1H), 2.78-2.65 (m, 1H), 2.61-2.47 (m, |
| | 5H), 2.47-2.26 (m, 2H), 2.11-1.97 (m, 1H), 1.94-1.81 (m, 1H), 1.81-1.64 |
| | (m, 2H), 1.15 (t, J = 7.3 Hz, 3H). 1 NH signal is not observed. |
| I-560 | MS m/z 421.4 [M + H]⁺; ¹H NMR (500 MHz, MeOD) δ 7.14 (s, 1H), 7.04 (d, J = |
| | 1.6 Hz, 1H), 6.30 (s, 1H), 4.47 (dd, J = 8.8, 3.8 Hz, 1H), 3.18-3.10 (m, 1H), |
| | 2.88-2.68 (m, 1H), 2.50 (s, 3H), 2.32-2.42 (m, 5H), 2.15 (s, 3H), 2.04 (d, J = |
| | 13.8 Hz, 1H), 1.89 (dt, J = 14.5, 4.6 Hz, 1H), 1.83-1.63 (m, 2H). NH and OH |
| | peak not observed. |
| I-579 | MS m/z 405, 407 [M + H]⁺; ¹H NMR (400 MHz, MeOD) δ 6.89-6.77 (m, 2H), |
| | 6.42 (s, 1H), 4.54-4.42 (m, 1H), 3.21-3.10 (m, 1H), 2.85-2.70 (m, 1H), |
| | 2.57 (q, J = 7.4 Hz, 2H), 2.52 (s, 3H), 2.48-2.33 (m, 2H), 2.11-2.00 (m, 1H), |
| | 1.95-1.82 (m, 1H), 1.82-1.66 (m, 2H), 1.16 (t, J = 7.2 Hz, 3H). 1 OH and 1 |
| | NH signals are not observed. |
| I-584 | MS m/z 437 [M + H]⁺; ¹H NMR (400 MHz, MeOD) δ 7 7.87 (br dd, J = 5.9, 1.4 |
| | Hz, 1H), 7.28 (br s, 1H), 6.92-7.10 (m, 2H), 4.54-4.75 (m, 1H), 3.86-3.97 (m, |
| | 1H), 3.62-3.71 (m, 1H), 2.95-3.07 (m, 2H), 2.64 (br s, 3H), 2.27-2.44 (m, 1H), |
| | 2.13-2.26 (m, 1H), 1.87-2.09 (m, 2H), 1.38-1.46 (m, 2H), 1.15-1.33 (m, 3H)1 |
| | OH and 1 NH signals are not observed. |
| I-585 | MS m/z 473.3 [M + H]⁺; ¹H NMR (400 MHz, MeOD) δ 8.19 (s, 1H), 8.13 (d, J = |
| | 8.5 Hz, 1H), 7.87 (d, J = 8.5 Hz, 1H), 6.36 (s, 1H), 4.55-4.43 (m, 1H), |
| | 3.12-3.03 (m, 1H), 2.77-2.66 (m, 1H), 2.62-2.48 (m, 5H), 2.48-2.30 (m, 2H), |
| | 2.10-1.99 (m, 1H), 1.93-1.81 (m, 1H), 1.81-1.65 (m, 2H), 1.15 (t, J = 7.3 |
| | Hz, 3H). 1 NH signal is not observed. |

### Example 49

### Preparation of Compounds I-332, I-336, and I-338

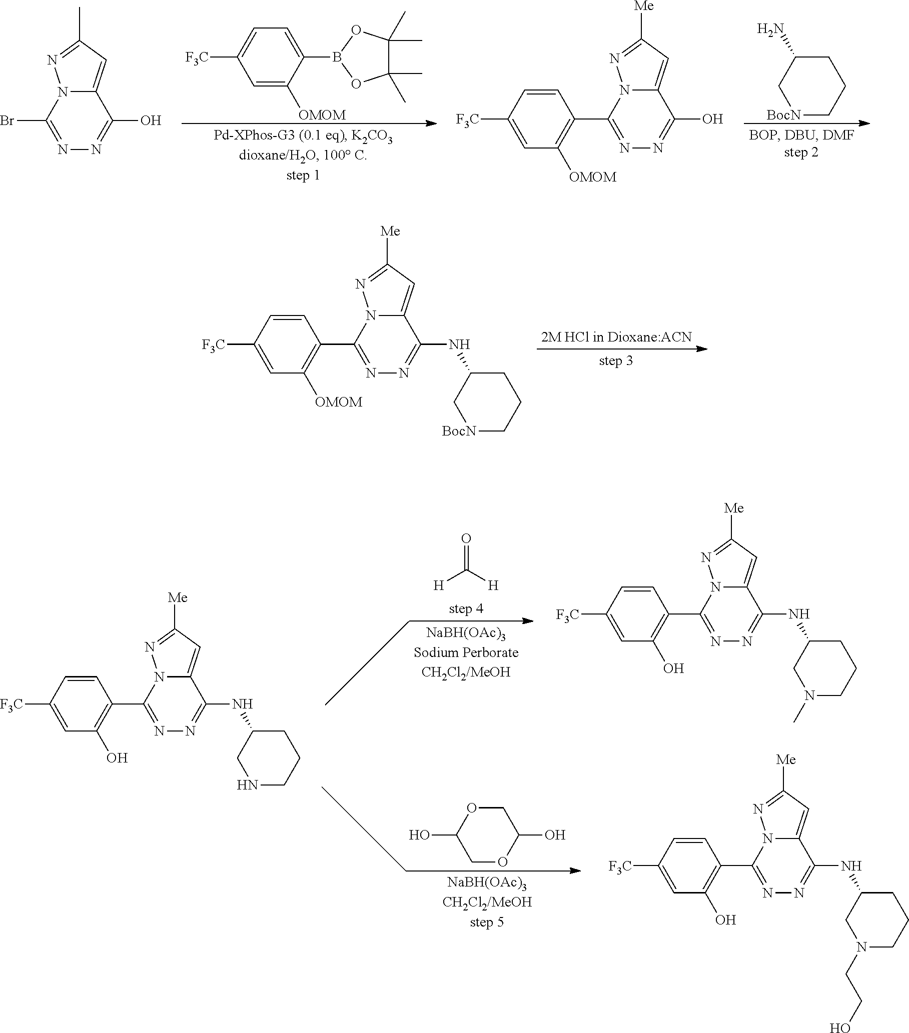

### Step 1. 7-(2-(Methoxymethoxy)-4-(trifluoromethyl)phenyl)-2-methylpyrazolo[1,5-d][1,2,4]triazin-4-ol

A mixture of 7-bromo-2-methylpyrazolo[1,5-d][1,2,4]triazin-4-ol (Intermediate 4a, 3.50 g, 15.3 mmol, 1.0 eq.), 2-(2-(methoxymethoxy)-4-(trifluoromethyl)phenyl)-4,4,5,5-tetramethyl-1,3,2-dioxaborolane (10.2 g, 30.6 mmol, 2.0 eq.), XPhosPdG₃ (1.30 g, 1.53 mmol, 0.1 eq.) and potassium carbonate (6.34 g, 45.9 mmol, 3.0 eq.) in water (7 mL) and 1,4-dioxane (35 mL) was heated at 100° C. for 16 h under nitrogen. Upon completion, the reaction mixture was cooled to room temperature and diluted with water (150 mL). The mixture was extracted with EtOAc (150 mL×3). The organic phase was dried over Na₂SO₄, filtered and concentrated under reduced pressure. The crude residue was purified by silica gel column chromatography eluting with 0-50% EtOAc in hexanes to afford 7-(2-(methoxymethoxy)-4-(trifluoromethyl)phenyl)-2-methylpyrazolo[1,5-d][1,2,4]triazin-4-ol (1.60 g, 4.52 mmol, 29.5% yield) as an off-white solid. MS m/z 355.1 [M+H]⁺. ¹H NMR (400 Hz, DMSO-d6) δ:12.62 (s, 1H), 7.77 (d, J=7.7 Hz, 1H), 7.57 (s, 1H), 7.53 (d, J=8.1 Hz, 1H), 7.07 (s, 1H), 5.26 (s, 2H), 3.26 (s, 3H), 2.31 (s, 3H).

### Step 2. tert-Butyl (R)-3-((7-(2-(methoxymethoxy)-4-(trifluoromethyl)phenyl)-2-methyl pyrazolo[1,5-d][1,2,4]triazin-4-yl)amino)piperidine-1-carboxylate

To a mixture of 7-(2-(methoxymethoxy)-4-(trifluoromethyl)phenyl)-2-methylpyrazolo[1,5-d][1,2,4]triazin-4-ol (248 mg, 0.70 mmol, 1 eq), tert-butyl (3R)-3-aminopiperidine-1-carboxylate (421 mg, 2.10 mmol, 3 eq) and 1,8-Diazabicyclo[5.4.0]undec-7-ene (0.28 mL, 1.75 mmol, 2.5 eq) in DMF (4.7 mL) was added BOP (358 mg, 0.77 mmol, 1.1 eq) at 0° C. The reaction mixture was allowed to slowly return to room temperature and stirred overnight. Upon completion, the reaction mixture was diluted with a small amount of NaHCO₃ (sat. aq.), extracted several times with EtOAc and the combined organic extracts were dried over Na₂SO₄ and concentrated in vacuo. The crude residue was purified first by silica gel column chromatography eluting with 0:100 to 10:90 MeOH:DCM to afford tert-butyl (R)-3-((7-(2-(methoxymethoxy)-4-(trifluoromethyl)phenyl)-2-methylpyrazolo[1,5-d][1,2,4]triazin-4-yl)amino)piperidine-1-carboxylate as an impure oil which was used without further purification.⁺

### Step 3. (R)-2-(2-Methyl-4-(piperidin-3-ylamino)pyrazolo[1,5-d][1,2,4]triazin-7-yl)-5-(trifluoromethyl)phenol

The product from step 2 was suspended in ACN (4 mL) and 4 M HCl in Dioxane (2 mL) and stirred at room temperature for 2 h. Upon reaction completion solvents were removed in vacuo. The residue was concentrated several times from dichloromethane to remove excess HCl. The crude residue was purified first by silica gel column chromatography eluting with 0:100 to 20:80 (10% NH₄OH in MeOH):DCM to afford (R)-2-(2-methyl-4-(piperidin-3-ylamino)pyrazolo[1,5-d][1,2,4]triazin-7-yl)-5-(trifluoromethyl)phenol (176 mg, 53% over two steps). MS m/z 393.4 [M+H]⁺; ¹H NMR (400 MHz, CD₃OD) δ 8.95 (d, J=8.3 Hz, 1H), 7.27-7.19 (m, 2H), 6.97 (s, 1H), 4.28-4.14 (m, 1H), 3.45-3.31 (m, 1H), 3.07-2.94 (m, 1H), 2.75-2.56 (m, 2H), 2.53 (s, 3H), 2.25-2.12 (m, 1H), 1.91-1.77 (m, 1H), 1.78-1.60 (m, 2H), three exchangeable protons not observed.

### Step 4. (R)-2-(2-Methyl-4-((1-methylpiperidin-3-yl)amino)pyrazolo[1,5-d][1,2,4]triazin-7-yl)-5-(trifluoromethyl)phenol

A mixture of (R)-7-(2-(methoxymethoxy)-4-(trifluoromethyl)phenyl)-2-methyl-N-(piperidin-3-yl)pyrazolo[1,5-d][1,2,4]triazin-4-amine (70 mg, 0.18 mmol, 1 eq) and sodium perborate tetrahydrate (82 mg, 0.54 mmol, 3 eq) in DCM (2 mL) and methanol (400 μL) was stirred for 30 min at 0° C. After 30 min, formaldehyde (37 wt % in water) (40 μL, 0.54 mmol, 3 eq) followed by sodium triacetoxyborohydride (113 mg, 0.54 mmol, 3 eq). The reaction was stirred at 0° C. for 1 min. The ice bath was removed, and the reaction mixture was stirred for 2 min. The reaction was quenched by addition a small amount of NaHCO₃ (sat, aq.) and a small amount of water followed by extraction with DCM. The combined organic extracts were dried over sodium sulfate and the crude material was purified by flash column chromatography 0:100 to 10:90 (10% NH₄OH in MeOH):DCM to afford (R)-2-(2-methyl-4-((1-methylpiperidin-3-yl)amino)pyrazolo[1,5-d][1,2,4]triazin-7-yl)-5-(trifluoromethyl)phenol (45 mg, 62%). MS m/z 407.5 [M+H]⁺; ¹H NMR (400 MHz, CD₃OD) δ 9.01 (d, J=8.5 Hz, 1H), 7.28-7.20 (m, 2H), 6.96 (s, 1H), 4.45-4.31 (m, 1H), 3.17-3.05 (m, 1H), 2.82-2.65 (m, 1H), 2.54 (s, 3H), 2.35 (s, 3H), 2.28-2.15 (m, 2H), 2.13-2.00 (m, 1H), 1.91-1.67 (m, 2H), 1.62-1.47 (m, 1H), NH and OH not observed.

### Step 5. (R)-2-(4-((1-(2-Hydroxyethyl)piperidin-3-yl)amino)-2-methylpyrazolo[1,5-d][1,2,4]triazin-7-yl)-5-(trifluoromethyl)phenol

1,4-Dioxane-2,5-diol (20 mg, 0.16 mmol, 1 eq) followed by sodium triacetoxyborohydride (102 mg, 0.48 mmol, 3 eq) were added to a solution of (R)-7-(2-(methoxymethoxy)-4-(trifluoromethyl)phenyl)-2-methyl-N-(piperidin-3-yl)pyrazolo[1,5-d][1,2,4]triazin-4-amine (63 mg, 0.16 mmol, 1 eq) in DCM (2 mL) and methanol (400 μL) at 0° C. The ice bath was removed, and the mixture was stirred at room temperature for 30 min. The reaction was quenched by addition a small amount of NaHCO₃(sat. aq.) and a small amount of water followed by extraction with DCM. The combined organic extracts were dried over sodium sulfate and the crude material was purified by flash column chromatography 0:100 to 20:80 (10% NH₄OH in MeOH):DCM to afford (R)-2-(4-((1-(2-hydroxyethyl)piperidin-3-yl)amino)-2-methylpyrazolo[1,5-d][1,2,4]triazin-7-yl)-5-(trifluoromethyl)phenol (48 mg, 69%). MS m/z 437.5 [M+H]⁺; ¹H NMR (400 MHz, CD₃OD) δ 9.02 (d, J=8.5 Hz, 1H), 7.29-7.18 (m, 2H), 7.00 (s, 1H), 4.44-4.31 (m, 1H), 3.79-3.64 (m, 2H), 3.21-3.06 (m, 1H), 2.80-2.67 (m, 1H), 2.67-2.48 (m, 5H), 2.45-2.26 (m, 2H), 2.09-1.93 (m, 1H), 1.90-1.79 (m, 1H), 1.79-1.55 (m, 2H), NH and 2 OH not observed.

The compounds below were prepared according to the procedure of Example 49 by substituting the appropriate starting materials, reagents and reaction conditions.

| Compound | Spectral Data |
|---|---|
| I-339 | MS m/z 355.4 [M + H]⁺; ¹H NMR (400 MHz, Methanol-d4) δ 8.97 (d, J = 9.0 |
| | Hz, 1H), 6.94 (s, 1H), 6.57 (s, 2H), 4.43-4.30 (m, 1H), 3.85 (s, 3H), 3.72-3.62 |
| | (m, 1H), 3.37-3.32 (m, 1H), 3.01-2.86 (m, 2H), 2.55 (s, 3H), 2.28-2.18 |
| | (m, 1H), 2.13-2.03 (m, 1H), 1.97-1.73 (m, 2H). NH and OH not observed |
| I-351 | MS m/z 369.4 [M + H]⁺; ¹H NMR (400 MHz, Methanol-d4) δ 9.04 (d, J = 8.8 |
| | Hz, 1H), 6.93 (s, 1H), 6.61-6.51 (m, 2H), 4.39-4.27 (m, 1H), 3.84 (s, 3H), |
| | 3.19-3.03 (m, 1H), 2.81-2.67 (m, 1H), 2.55 (s, 3H), 2.35 (s, 3H), 2.32-2.14 |
| | (m, 2H), 2.13-1.97 (m, 1H), 1.93-1.67 (m, 2H), 1.65-1.44 (m, 1H). |

### Example 50

### Preparation of Compound I-546

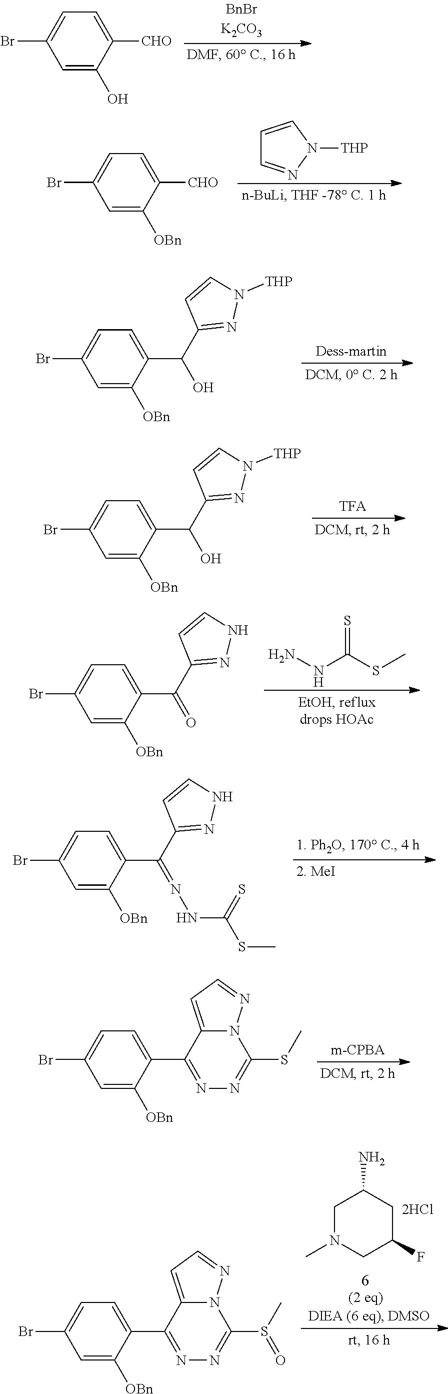

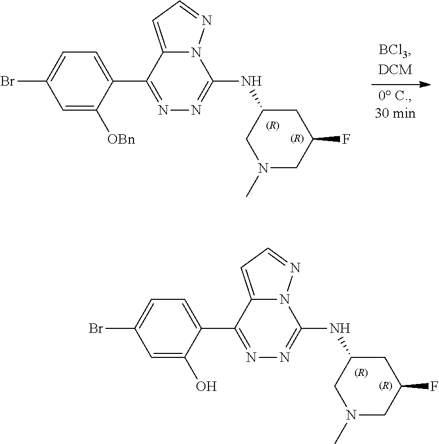

### Step 1. 2-(Benzyloxy)-4-bromobenzaldehyde

To a solution of compound 4-bromo-2-hydroxy-benzaldehyde (10.0 g, 49.7 mmol) in DMF (100 mL) was added (bromomethyl)benzene (10.2 g, 59.6 mmol) and K₂CO₃(17.2 g, 124 mmol). The reaction mixture was stirred at 60° C. under N₂ for 16 hours, then cooled to room temperature. The mixture was filtered and the filtrate was concentrated under reduce pressure. The residue was diluted with DCM (150 mL) and washed with brine (2×50 mL), dried over anhydrous Na₂SO₄, evaporated in vacuum, and the crude product was purified on silica gel eluted with (PE/EA=1:10) to give the compound 2-benzyloxy-4-bromo-benzaldehyde (14.0 g, 96.7% Yield) as a white solid.

### Step 2. (2-(Benzyloxy)-4-bromophenyl)(1-(tetrahydro-2H-pyran-2-yl)-1H-pyrazol-3-yl)methanol

To a solution of 1-(tetrahydro-2H-pyran-2-yl)-1H-pyrazole (12.34 g, 80.27 mmol) in THE (300 mL) was added n-BuLi in hexanes (46.5 mL, 120 mmol, 2.5 mol/L) at −65° C. over 30 min. The mixture was stirred at 0-10° C. for 10-20 min. Then a solution of 2-benzyloxy-4-bromo-benzaldehyde (19.6 g, 67.3 mmol) in THE (100 mL) was added dropwise at −65° C. for 30 min. The reaction mixture was stirred at −65° C. under N₂ for 2 hours, the mixture was quenched by sat. NH₄Cl (150 mL) and extracted with EtOAc(100 mL×3). The organic layer was combined and washed with brine (50 mL×2), dried with Na₂SO₄, filtered and concentrated under reduced pressure. The crude product was purified on silica gel eluted with (PE/EA=1:10) to give the compound (2-(benzyloxy)-4-bromophenyl)(1-(tetrahydro-2H-pyran-2-yl)-1H-pyrazol-3-yl)methanol (13.0 g, 43.8% Yield) as a pale yellow oil. MS m/z 443, 445 [M+H]⁺.

### Step 3. (2-(Benzyloxy)-4-bromophenyl)(1-(tetrahydro-2H-pyran-2-yl)-1H-pyrazol-3-yl)methanone

To a solution of (2-benzyloxy-4-bromo-phenyl)-(1-tetrahydropyran-2-ylpyrazol-3-yl)methanol (13.0 g, 29.41 mmol) in DCM (130 mL) was added Dess-Martin (18.7 g, 44.11 mmol) under N₂ at 0° C. for 2 h. The sat. NaHCO₃ (200 mL) was added and extracted with DCM (250 mL×2). the organic layer was combined and washed with brine (100 mL), dried with Na₂SO₄, filtered and concentrated under reduced pressure. The crude product was purified on silica gel eluted with (PE/EA=1:4) to give the compound (2-(benzyloxy)-4-bromophenyl)(1-(tetrahydro-2H-pyran-2-yl)-1H-pyrazol-3-yl)methanone (9.3 g, 43.8% Yield) as a pale yellow oil. MS m/z 441, 443 [M+H]⁺.

### Step 4. (2-(Benzyloxy)-4-bromophenyl)(1-(tetrahydro-2H-pyran-2-yl)-1H-pyrazol-3-yl)methanone

To a solution of compound (2-benzyloxy-4-bromo-phenyl)-(1-tetrahydropyran-2-ylpyrazol-3-yl)methanone (9.3 g, 21.1 mmol) in DCM (80 mL) was added TFA (20 mL, 264.5 mmol). The reaction mixture was stirred at RT for 2 h. The solvents was removed under reduce pressure. The residue was diluted with DCM (150 mL) and washed with saturated NaHCO₃(2×50 mL), dried over anhydrous Na₂SO₄, filtered and concentrated under reduced pressure to give the compound (2-(benzyloxy)-4-bromophenyl)(1H-pyrazol-3-yl)methanone (5.0 g, 43.8% Yield) as a pale yellow oil. The crude product could be used next step without further purification. MS m/z 357, 359 [M+H]⁺.

### Step 5. Methyl 2-((2-(benzyloxy)-4-bromophenyl)(1H-pyrazol-3-yl)methylene)hydrazine-1-carbodithioate

To a solution of (2-(benzyloxy)-4-bromophenyl)(1H-pyrazol-3-yl)methanone (2.5 g, 7.02 mmol) and methyl hydrazinecarbodithioate (1.7 g, 14.04 mmol) in EtOH (30 mL), was added CH₃COOH (0.1 mL) under N₂ was stirred at 80° C. for 4 h. The reaction solution was concentrated in vacuo to obtain a crude product methyl 2-((2-(benzyloxy)-4-bromophenyl)(1H-pyrazol-3-yl)methylene)hydrazine-1-carbodithioate (2.6 g, 80.5% Yield) as a pale yellow oil, which is used directly for the next step. MS m/z 461, 463 [M+H]⁺.

### Step 6. 4-(2-(Benzyloxy)-4-bromophenyl)-7-(methylthio)pyrazolo[1,5-d][1,2,4]triazine

A solution of methyl 2-((2-(benzyloxy)-4-bromophenyl)(1H-pyrazol-3-yl)methylene)hydrazine-1-carbodithioate (2.6 g, 5.65 mmol) in Ph₂O (30 mL) was stirred at 170° C. for 4 h. The reaction mixture was cooled to room temperature. MeI (2.0 g, 14.12 mmol) and K₂CO₃ (1.95 g, 14.12 mmol, 100 mass %) in THE (20 mL) and H₂O (10 mL) was then added to the mixture and the reaction mixture was stirred at rt for 2 h. 50 ml of water was then added. The mixture was extracted with EtOAc (2×80 mL). The organic layer was combined, dried over Na₂SO₄, evaporated in vacuo. The crude product was purified by silica gel chromatography (PE/EA=1/1) to afford desire product 4-(2-(benzyloxy)-4-bromophenyl)-7-(methylthio)pyrazolo[1,5-d][1,2,4]triazine (1.02 g, 42.3% Yield) as a pale yellow solid. MS m/z 427.0, 429.0 [M+H]⁺; ¹H NMR (400 MHz, DMSO-d6) δ 8.35 (d, J=2.2 Hz, 1H), 7.59 (d, J=1.7 Hz, 1H), 7.49 (d, J=8.1 Hz, 1H), 7.38 (dd, J=8.1, 1.8 Hz, 1H), 7.23 (ttd, J=9.7, 4.9, 1.8 Hz, 5H), 6.97 (d, J=2.2 Hz, 1H), 5.22 (s, 2H), 2.80 (d, J=7.6 Hz, 3H).

### Step 7. 4-(2-(Benzyloxy)-4-bromophenyl)-7-(methylsulfinyl)pyrazolo[1,5-d][1,2,4]triazine

To a solution of 4-(2-benzyloxy-4-bromo-phenyl)-7-methylsulfanyl-pyrazolo[1,5-d][1,2,4]triazine (50 mg, 0.12 mmol) in DCM (2 mL) was added m-CPBA (24.5 mg, 0.14 mmol) at 0° C. under N₂. Then the reaction mixture was stirred at rt for 2 h. LCMS showed 15% starting materials was remained. m-CPBA (33 mg, 0.08 mmol) was added. The reaction mixture was stirred at rt for 2 h. 20 ml of saturated NaHCO₃ was added. The mixture was extracted with DCM (2×20 mL). The organic phase was dried over Na₂SO₄, evaporated in vacuum to give a pale-yellow oil (45 mg). The crude product could be used next step without further purification. MS m/z 443.1, 445.1 [M+H]⁺.

### Step 8. 4-(2-(Benzyloxy)-4-bromophenyl)-N-((3R,5R)-5-fluoro-1-methylpiperidin-3-yl)pyrazolo[1,5-d][1,2,4]triazin-7-amine

To a solution of 4-(2-benzyloxy-4-bromo-phenyl)-7-methylsulfinyl-pyrazolo[1,5-d][1,2,4]triazine (45 mg, 0.10 mmol) in DMSO (1 mL) was added (3R,5R)-5-fluoro-1-methyl-piperidin-3-amine hydrochloride (35 mg, 0.20 mmol) and DIEA (79 mg, 0.60 mmol) under N₂. Then the reaction mixture was stirred at rt for 16 h. Saturated aq. NaHCO₃ (20 mL) was added. The mixture was extracted with EtOAc (2×20 mL). The organic layer was combined, dried over Na₂SO₄, and evaporated in vacuum. The crude product was purified by prep-HPLC (0.1% FA/H₂O:CH₃CN) 5% CH₃CN to 95% CH₃CN to give 19 mg of target compound. MS m/z 511.2, 513.2 [M+H]⁺.

### Step 9. 5-Bromo-2-(7-(((3R,5R)-5-fluoro-1-methylpiperidin-3-yl)amino)pyrazolo[1,5-d][1,2,4]triazin-4-yl)phenol

To a solution of 4-(2-(benzyloxy)-4-bromophenyl)-N-((3R,5R)-5-fluoro-1-methylpiperidin-3-yl)pyrazolo[1,5-d][1,2,4]triazin-7-amine (40 mg, 0.078 mmol) in DCM (2 mL) was added BCl₃(1.0M solution in hexane) (0.5 ml) at 0° C. The reaction mixture was stirred at 0° C. for 30 min. LCMS showed the reaction completed. MeOH (0.5 mL) was slowly added under 0° C. and saturated NaHCO₃ (15 mL) was added. The mixture was extracted with DCM/MeOH=10:1 (2×20 mL), dried over Na₂SO₄, and evaporated in vacuum, The crude product was purified by prep-HPLC (0.1% FA/H₂O:CH₃CN) 5% CH₃CN to 95% CH₃CN to give 4.3 mg of target compound as a pale yellow solid. MS m/z 421.0, 423.0 [M+H]⁺; ¹H NMR (400 MHz, CD₃OD) δ 8.27 (d, J=2.0 Hz, 1H), 7.85 (d, J=8.4 Hz, 1H), 7.27 (d, J=2.2 Hz, 1H), 7.21-7.10 (m, 2H), 4.99 (d, J=46 Hz, 1H), 4.79-4.73 (m, 1H), 3.21 (d, J=11.6 Hz, 1H), 3.02 (t, J=11.6 Hz, 1H), 2.57-2.44 (m, 1H), 2.42 (s, 3H), 2.38-231 (m, 2H), 2.09-1.93 (m, 1H), NH and OH not observed.

### Example 51

### Preparation of Compound I-634

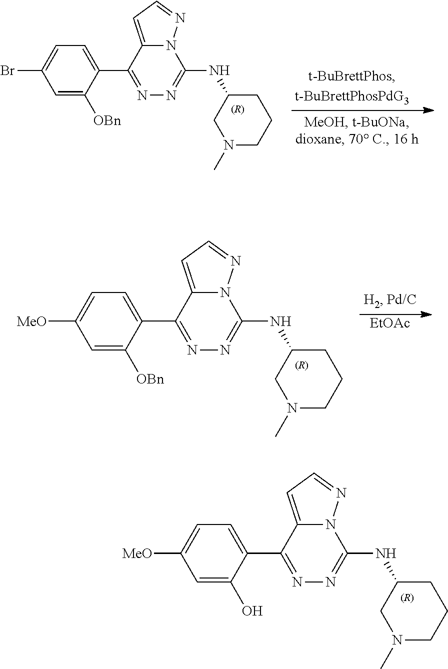

### Step 1. (R)-4-(2-(Benzyloxy)-4-methoxyphenyl)-N-(1-methylpiperidin-3-yl)pyrazolo[1,5-d][1,2,4]triazin-7-amine

The starting material, (R)-4-(2-(benzyloxy)-4-bromophenyl)-N-(1-methylpiperidin-3-yl)pyrazolo[1,5-d][1,2,4]triazin-7-amine, was prepared in analogous manner according to the procedure of Example 50, using (R)-1-methylpiperidin-3-amine hydrochloride in place of (3R,5R)-5-fluoro-1-methyl-piperidin-3-amine hydrochloride in step 8.

To a solution of (R)-4-(2-(benzyloxy)-4-bromophenyl)-N-(1-methylpiperidin-3-yl)pyrazolo[1,5-d][1,2,4]triazin-7-amine (43 mg, 0.09 mmol) in dioxane (1 mL) was added t-BuBrettPhos (4.8 mg, 0.01 mmol) t-BuBrettPhosPdG3 (8.5 mg, 0.01 mmol), sodium tert-butoxide (26 mg, 0.27 mmol) and MeOH (29 mg, 0.9 mmol) under N₂. Then the reaction mixture was stirred at 70° C. for 16 h. Water (10 mL) was added and the mixture was extracted with EtOAc (2×10 mL). The organic layer was combined, washed with brine, dried over Na₂SO₄, and evaporated in vacuum. The crude product was purified on silica gel eluted with (DCM/MeOH=20:1) to give the compound 4-(2-(benzyloxy)-4-bromophenyl)-7-(methylsulfinyl)pyrazolo[1,5-d][1,2,4]triazine (58 mg, 74.3% Yield) as a pale yellow oil. MS m/z 445.3 [M+H]⁺.

### Step 2. (R)-5-Methoxy-2-(7-((1-methylpiperidin-3-yl)amino)pyrazolo[1,5-d][1,2,4]triazin-4-yl)phenol

To a solution of 4-(2-(benzyloxy)-4-bromophenyl)-7-(methylsulfinyl)pyrazolo[1,5-d][1,2,4]triazine (135 mg, 0.30 mmol) in EtOAc (3 mL) was added Pd/C (10%) (32 mg, 0.03 mmol) under N₂. Then the atmosphere was changed with H₂ three times. The reaction mixture was stirred at r.t. for 2 h, then filtered. The filtrate was concentrated under reduce pressure. The crude was purified by Pre-HPLC (0.1% FA/H₂O:CH₃CN) to give (R)-5-methoxy-2-(7-((1-methylpiperidin-3-yl)amino)pyrazolo[1,5-d][1,2,4]triazin-4-yl)phenol (5.8 mg, 10.9% Yield) as a pale yellow solid. MS m/z 355.3 [M+H]⁺; ¹H NMR (400 MHz, CDM₃OD) δ 8.16 (d, J=2.0 Hz, H), 7.81 (d, J=8.8 Hz, 8H), 7.21 (d, J=2.0 Hz, H), 6.52 (dd, J 8.8, 2.6 Hz, 1H), 6.46 (d, J=2.4 Hz, 1H), 4.39-4.22 (s, 1H), 3.74 (s, 3H), 3.06-2.95 (m, 1H), 2.70-2.55 (d, 1H), 2.43-2.21 (m, 5H), 1.99-1.90 (1H), 1.79 (dd, J=9.5, 3.8 Hz, 1H), 1.70-1.55 (m, 2H), NH and OH not observed.

The compounds below were prepared according to the procedure of Example 50 by substituting the appropriate starting materials, reagents and reaction conditions.

| Compound | Spectral Data |
|---|---|
| I-390 | MS m/z 403.0, 405.0 [M + H]⁺; ¹H NMR (400 MHz, DMSO-d6) δ 9.00 (d, J = 64 |
| | Hz, 2H), 8.28 (dd, J = 17.7, 5.2 Hz, 2H), 7.43 (d, J = 1.6 Hz, 1H), 7.41 (d, J = |
| | 8.0 Hz, 1H), 7.31 (dd, J = 8.1, 1.6 Hz, 1H), 6.67 (d, J = 2.0 Hz, 1H), 4.74-4.43 |
| | (m, 1H), 3.87 (s, 1H), 3.80 (s, 3H), 3.52 (d, J = 10.4 Hz, 1H), 3.28 (d, J = 12.4 |
| | Hz, 1H), 3.11 (d, J = 10.0 Hz, 1H), 2.88 (d, J = 9.2 Hz, 1H), 2.09 (dd, J = 8.7, |
| | 3.6 Hz, 1H), 1.95 (dd, J = 18.0, 8.4 Hz, 1H), 1.90-1.72 (m, 2H). |
| I-391 | MS m/z 390.0 [M + H]⁺; ¹H NMR (400 MHz, DMSO-d6) δ 8.42 (dd, J = 18.8, |
| | 4.7 Hz, 2H), 7.78 (d, J = 8.4 Hz, 1H), 7.21 (ddd, J = 10.2, 9.3, 1.9 Hz, 3H), 4.55 |
| | (s, 1H), 3.46 (d, J = 12.2 Hz, 2H), 3.20 (s, 1H), 3.06-2.96 (m, 1H), 2.83 (t, J = |
| | 10.8 Hz, 1H), 2.04 (s, 1H), 1.83 (ddd, J = 37.5, 24.7, 13.6 Hz, 3H). |
| I-451 | MS m/z 417.0, 419.0[M + H]⁺; ¹H NMR (400 MHz, CD₃OD) δ 8.45 (s, 1H), 8.19 |
| | (d, J = 2.1 Hz, 1H), 7.41 (d, J = 8.2 Hz, 2H), 7.31 (dd, J = 8.1, 1.5 Hz, 1H), 6.66 |
| | (d, J = 2.1 Hz, 1H), 4.71-4.51 (m, 1H), 3.83 (s, 3H), 3.70-3.56 (m, 1H), 3.30 |
| | (s, 1H), 3.02 (d, J = 22.7 Hz, 1H), 2.94 (s, 1H), 2.83 (s, 3H), 2.25-2.08 (m, |
| | 2H), 2.03-1.81 (m, 2H). NH not observed |
| I-452 | MS m/z 403.0, 405.0[M + H]⁺; ¹H NMR (400 MHz, CD₃OD) δ 8.50 (s, 1H), 8.28 |
| | (d, J = 2.2 Hz, 1H), 7.83 (d, J = 8.4 Hz, 1H), 7.26 (d, J = 2.2 Hz, 1H), 7.17 (dt, |
| | J = 8.3, 2.0 Hz, 2H), 4.50 (d, J = 4.0 Hz, 1H), 3.32 (s, 1H), 2.97 (s, 1H), 2.69 (d, |
| | J = 7.2 Hz, 1H), 2.65-2.49 (m, 4H), 2.13 (s, 1H), 1.98 (d, J = 4.8 Hz, 1H), |
| | 1.82 (dd, J = 14.0, 10.4 Hz, 2H). NH and OH not observed |
| I-453 | MS m/z 417.1, 419.1 [M + H]⁺; ¹H NMR (400 MHz, CD₃OD) δ 8.28 (d, J = 2.2 |
| | Hz, 1H), 7.84 (d, J = 8.4 Hz, 1H), 7.27 (d, J = 2.2 Hz, 1H), 7.22-7.14 (m, 2H), |
| | 4.49 (s, 1H), 3.28-3.15 (m, 1H), 2.92 (s, 1H), 2.71 (q, J = 7.0 Hz, 2H), 2.62 (s, |
| | 2H), 2.11 (s, 1H), 1.94 (s, 1H), 1.81 (d, J = 10.0 Hz, 2H), 1.20 (t, J = 7.2 Hz, |
| | 3H). NH and OH not observed |
| I-544 | MS m/z: 390.0, 392.0 [M + H]⁺; ¹H NMR (400 MHz, MeOD) δ 8.28 (d, J = 2.1 |
| | Hz, 1H), 7.85 (d, J = 8.4 Hz, 1H), 7.41-6.97 (m, 3H), 4.47-4.16 (m, 1H), |
| | 2.92-2.42 (m, 2H), 2.31 (t, J = 10.1 Hz, 2H). NH and OH not observed |
| I-545 | MS m/z: 493.0, 495.0 [M + H]⁺; ¹H NMR (400 MHz, MeOD) δ 8.15 (d, J = 2.1 |
| | Hz, 1H), 7.50 (d, J = 1.7 Hz, 1H), 7.43 (d, J = 8.1 Hz, 1H), 7.34 (dd, J = 8.1, 1.7 |
| | Hz, 1H), 7.27-7.18 (m, 5H), 6.72 (d, J = 2.1 Hz, 1H), 5.15 (s, 2H), 4.65-4.54 |
| | (m, 1H), 3.64 (s, 1H), 3.32-3.27 (m, 1H), 3.04 (d, J = 86.1 Hz, 2H), 2.82 (s, |
| | 3H), 2.30-2.04 (m, 2H), 1.99-1.80 (m, 2H). NH and OH not observed |
| I-547 | MS m/z: 390.0, 392.0 [M + H]⁺; ¹H NMR (400 MHz, MeOD) δ 8.28 (d, J = 2.2 |
| | Hz, 1H), 7.85 (d, J = 8.3 Hz, 1H), 7.27 (d, J = 2.2 Hz, 1H), 7.18 (dt, J = 8.4, 2.0 |
| | Hz, 2H), 4.35 (dd, J = 10.5, 6.5 Hz, 1H), 4.08 (dd, J = 11.1, 2.7 Hz, 1H), 3.83 |
| | (dd, J = 10.6, 5.6 Hz, 1H), 3.64-3.53 (m, 2H), 2.17 (s, 1H), 1.99-1.83 (m, |
| | 2H), 1.82-1.70 (m, 1H). NH and OH not observed |
| I-548 | MS m/z 453.0, 455.0 [M + H]⁺; ¹H NMR(400 MHz, MeOD) δ 8.28 (d, J = 2.2 Hz, |
| | 1H), 7.86 (d, J = 8.3 Hz, 1H), 7.28 (d, J = 2.2 Hz, 1H), 7.18 (dt, J = 8.3, 2.0 Hz, |
| | 2H), 6.01 (tt, J = 56.0, 4.3 Hz, 1H), 4.42 (s, 1H), 3.16-3.01 (m, 1H), 2.83 (td, |
| | J = 15.2, 4.3 Hz, 2H), 2.76-2.53 (m, 3H), 1.94 (s, 1H), 1.87-1.66 (m, 3H). NH |
| | and OH not observed |
| I-632 | MS m/z 369.2 [M + H]⁺; ¹H NMR (400 MHz, MeOD) δ 8.27 (d, J = 2.0 Hz, 1H), |
| | 7.90 (d, J = 8.8 Hz, 1H), 7.31 (d, J = 2.0 Hz, 1H), 6.62 (dd, J = 8.8, 2.4 Hz, 1H), |
| | 6.56 (d, J = 2.4 Hz, 1H), 4.60-4.46 (m, 1H), 3.84 (s, 3H), 3.70-3.57 (m, 1H), |
| | 3.36 (d, J = 6.8 Hz, 1H), 3.07 (q, J = 7.2 Hz, 2H), 3.00-2.77 (m, 2H), 2.25-2.06 |
| | (m, 2H), 2.00-1.80 (m, 2H), 1.32 (t, J = 7.2 Hz, 3H). NH and OH not observed |
| I-633 | MS m/z 353.3 [M + H]⁺; ¹H NMR (400 MHz, MeOD) δ 8.17 (d, J = 2.0 Hz, 1H), |
| | 7.73 (d, J = 7.8 Hz, 1H), 7.18 (d, J = 2.0 Hz, 1H), 6.76 (d, J = 8.4 Hz, 2H), |
| | 4.40 (s, 1H), 3.40-3.28 (m, 1H), 3.04-2.93 (m, 1H), 2.76 (q, J = 7.0 Hz, 2H), |
| | 2.65-2.47 (m, 2H), 2.26 (s, 3H), 2.09-2.01 (m, 1H), 1.95-1.88 (m, 1H), |
| | 1.81-1.67 (m, 2H), 1.15 (t, J = 7.2 Hz, 3H). NH and OH not observed |

### Example 52

### Preparation of Compounds I-349 and I-333

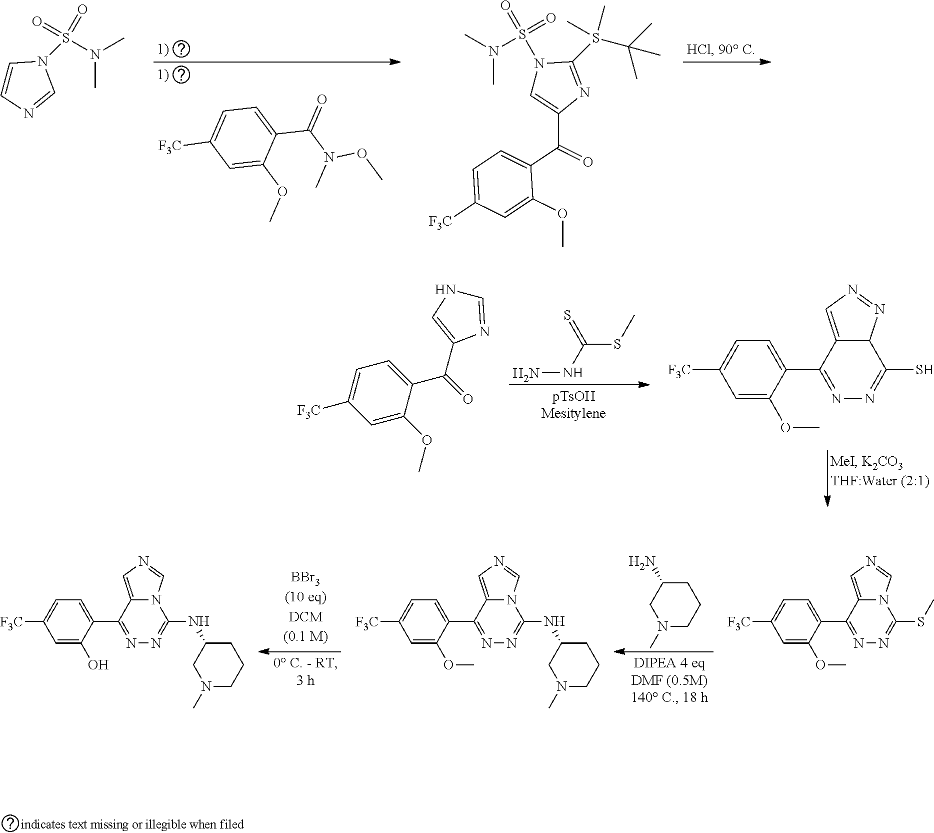

### Step 1: 1H-Imidazol-4-yl-[2-methoxy-4-(trifluoromethyl)phenyl]methanone

A solution of N,N-dimethylimidazole-1-sulfonamide (5.17 g, 29.5 mmol) in tetrahydrofuran (0.3 M, 98 mL) was cooled to −78 TC. n-Butyllithium (2.5 mmol/L) in hexanes (1.1 eq., 13 mL) was added slowly under argon. The reaction was stirred at the same temperature for 30 minutes. Then, tert-butyl-chloro-dimethyl-silane (1.1 eq., 4.89 g) in THE (<1 mL) was added dropwise. The reaction was allowed to warm up slowly to room temperature and continued to stir at room temperature for 10 minutes. The mixture was cooled to −78° C. and n-butyllithium (2.5 mol/L) in hexanes (1.1 eq., 13 mL) was added under argon. The reaction was warmed to −30° C. and stirred for 1 h. N, 2-dimethoxy-N-methyl-4-(trifluoromethyl)benzamide (0.75 eq., 5.83 g, 22.1 mmol) was added and the reaction was warmed to room temperature and stirred for 3 hours. The reaction was quenched with ammonium chloride solution. The mixture was partitioned between sat. aq. ammonium chloride and EtOAc and extracted with EtOAc (×2). The organics were dried over Na₂SO₄ and concentrated. The crude material was purified by flash column chromatography on silica gel eluting with 0-100% EtOAc in hexane to provide 1H-imidazol-4-yl-[2-methoxy-4-(trifluoromethyl)phenyl]methanone (7.0 g, 48% Yield). MS m/z 492.2 [M+H]⁺

### Step 2: 1H-Imidazol-4-yl-[2-methoxy-4-(trifluoromethyl)phenyl]methanone

Hydrochloric acid (1 mol/L) in water (0.2 M, 71.2 ml) was added to 2-[tert-butyl(dimethyl)silyl]-4-[2-methoxy-4-(trifluoromethyl)benzoyl]-N,N-dimethyl-imidazole-1-sulfonamide (7 g, 14.21 mmol). The resulting mixture was warmed to 90° C. and stirred for 1 h. The crude material was cooled to room temperature and neutralized with 1 N NaOH to pH-7. The mixture was extracted with EtOAc (×4). Combined organics were dried over Na₂SO₄ and concentrated. The crude material was applied to the next step without future purification. MS m/z 271.2 [M+H]⁺

### Step 3: 1-[2-Methoxy-4-(trifluoromethyl)phenyl]imidazo[1,5-d][1,2,4]triazine-4-thiol

A mixture of 1H-imidazol-4-yl-[2-methoxy-4-(trifluoromethyl)phenyl]methanone (3.0 g, 11 mmol), methyl N-aminocarbamodithioate (2 eq., 2.7 g, 22 mmol), p-toluenesulfonic acid (0.2 eq., 0.39 g, 2.2 mmol) and 1,3,5-trimethylbenzene (0.5 M, 22 mL) was heated at 160° C. for 1 hour. The UPLC indicated the reaction in completed. The mixture was cooled to room temperature, then partitioned between brine and EtOAc, extracted with EtOAc (×5). Combined organics were dried over Na₂SO₄ and concentrated. The crude material was purified by flash column chromatography on silica gel eluting with 0-100% EtOAc in hexane to provide

1-[2-methoxy-4-(trifluoromethyl)phenyl]imidazo[1,5-d][1,2,4]triazine-4-thiol (2.9 g, 80% yield). MS m/z 327.1 [M+H]⁺.

### Step 4: 1-[2-Methoxy-4-(trifluoromethyl)phenyl]-4-methylsulfanyl-imidazo[1,5-d][1,2,4]triazine

1-[2-Methoxy-4-(trifluoromethyl)phenyl]imidazo[1,5-d][1,2,4]triazine-4-thiol (3.2 g, 9.8 mmol) and potassium carbonate (2.5 g, 18 mmol) was mixed in tetrahydrofuran:water (2:1) (0.4 M, 25 mL). Then, iodomethane (2.5 g, 18 mmol) was added and the resulting mixture was stirred at room temperature for 1 h. The reaction mixture was partitioned between EtOAc and brine. After extraction, the organics were concentrated. The crude material was purified by flash column chromatography on silica gel eluting with 0-100% EtOAc in hexane to provide 1-[2-methoxy-4-(trifluoromethyl)phenyl]-4-methylsulfanyl-imidazo[1,5-d][1,2,4]triazine (2.43 g, 73% Yield). MS m/z 341.7 [M+H]⁺.

### Step 5: 1-[2-Methoxy-4-(trifluoromethyl)phenyl]-N-[(3R)-1-methyl-3-piperidyl]imidazo[1,5-d][1,2,4]triazin-4-amine

1-[2-Methoxy-4-(trifluoromethyl)phenyl]-4-methylsulfanyl-imidazo[1,5-d][1,2,4]triazine (150.0 mg, 0.4408 mmol) and (3R)-1-methylpiperidin-3-amine; trihydrochloride (2.0 eq., 0.19 g, 0.88 mmol) were mixed in N,N-diisopropylethylamine (10 eq., 0.76 mL, 4.4 mmol) and N,N-dimethylformamide (0.5 M, 0.8815 mL). The resulting mixture was warmed to 130° C., and stirred for 15 h. After cooling, the crude material was directly loaded on silica column and purified, purified by flash column Chromatography to afford 1-[2-methoxy-4-(trifluoromethyl) phenyl]-N-[(3R)-1-methyl-3-piperidyl]imidazo[1,5-d][1,2,4]triazin-4-amine (88 mg, 49.13% Yield). MS m/z 407.0 [M+H]⁺. ¹H NMR (METHANOL-d4) δ: 8.62 (s, 1H), 8.51 (br s, 1H, formic acid peak), 7.44-7.66 (m, 1H), 7.34 (br s, 3H), 4.26-4.54 (m, 1H), 3.78 (s, 3H), 3.26 (br d, J=0.8 Hz, 1H), 2.75-2.97 (m, 1H), 2.45 (br s, 5H), 1.96-2.14 (m, 1H), 1.82-1.98 (m, 1H), 1.67-1.81 (m, 1H), 1.46-1.66 (m, 1H), NH and OH not observed.

### Step 6: 2-[4-[[(3R)-1-Methyl-3-piperidyl]amino]imidazo[1,5-d][1,2,4]triazin-1-yl]-5-(trifluoromethyl)phenol

1-[2-Methoxy-4-(trifluoromethyl)phenyl]-N-[(3R)-1-methyl-3-piperidyl]imidazo[1,5-d][1,2,4]triazin-4-amine (88 mg, 0.21 mmol) in dichloromethane (1.1 mL) was cooled to 0° C. Tribromoborane (10 eq., 0.2 mL, 2.2 mmol) was added and the resulting mixture was warmed to room temperature and stirred for 3 h. The reaction mixture was then cooled to 0° C. and quenched with addition of K₂CO₃ and methanol carefully. After stirring for 30 minutes, the mixture was concentrated. The residue was then purified on prep-HPLC with 5-50% ACN in water with 0.1% formic acid to provide 2-[4-[[(3R)-1-methyl-3-piperidyl]amino]imidazo[1,5-d][1,2,4]triazin-1-yl]-5-(trifluoromethyl)phenol (18 mg, 21% Yield). MS m/z 393.1 [M+H]⁺; ¹H NMR (CD₃OD) δ: 8.73 (s, 1H), 8.10 (br s, 1H), 7.92-8.20 (m, 2H), 7.11-7.29 (m, 2H), 4.33-4.54 (m, 1H), 3.47-3.65 (m, 1H), 3.10-3.18 (m, 1H), 2.75-2.93 (m, 2H), 2.70 (s, 3H), 1.93-2.17 (m, 2H), 1.63-1.90 (m, 2H), NH and OH not observed.

### Example 53

### Preparation of Compound I-518

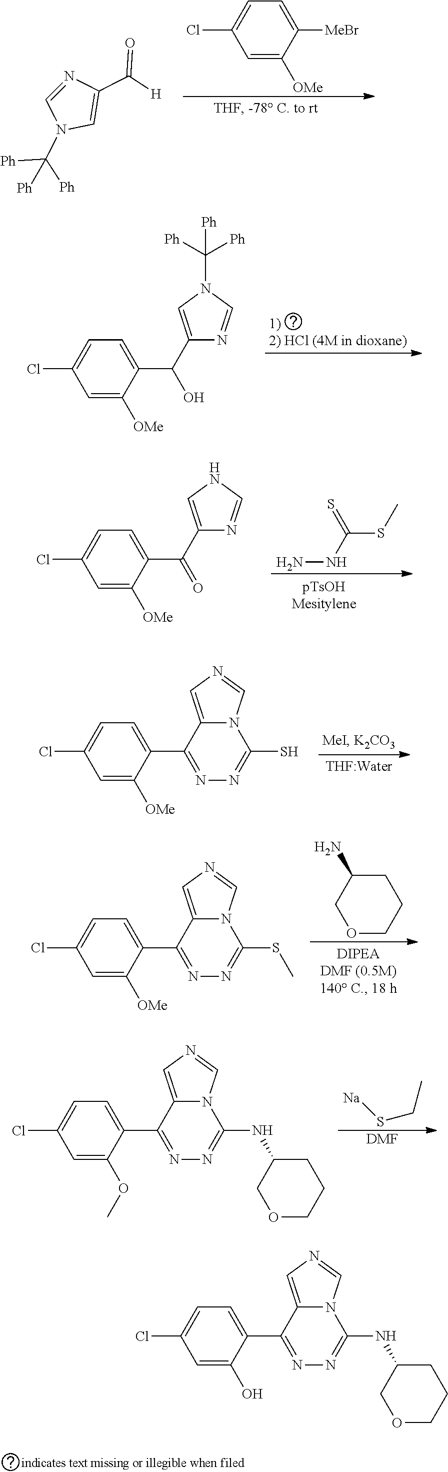

### Step 1: (4-Chloro-2-methoxyphenyl)(1-trityl-1H-imidazol-4-yl)methanol

To a suspension of 1-trityl-1H-imidazole-4-carbaldehyde (5 g, 14.8 mmol, 1 eq.) in THF was added (4-chloro-2-methoxyphenyl)magnesium bromide (40 mL, 1.2 eq., 0.5 M in THF) dropwise at −78° C. The reaction mixture was allowed to warm to room temperature and was stirred overnight. Upon completion, the reaction was quenched with water and a white solid crashed out. The precipitate was filtered and dried under vacuum. The crude material was collected as (4-chloro-2-methoxyphenyl)(1-trityl-1H-imidazol-4-yl)methanol (6.67 g, 94% yield) and was used in the next step without further purification.

### Step 2. (4-Chloro-2-methoxyphenyl)(1H-imidazol-4-yl)methanone

(4-Chloro-2-methoxyphenyl)(1-trityl-1H-imidazol-4-yl)methanol (1 g, 2 mmol, 1 eq.) was dissolved in minimum amount of DMSO, followed by addition of Dess-Martin reagents (2.2 g, 5.2 mmol, 2.5 eq.). The reaction was stirred at room temperature for 30 min. Then HCl (4 eq., 4 M in dioxane was added) to the reaction mixture slowly. The reaction was stirred at room temperature for another 30 min. Upon completion, the reaction was diluted with ethyl acetate and washed with DMSO. The organics were collected, dried over sodium sulphate, and concentrated under reduced pressure. The crude material was collected as (4-chloro-2-methoxyphenyl)(1H-imidazol-4-yl)methanone (372 mg, 76% yield) and was used in the next step without further purification. MS m/z 237.1, 239.1 [M+H]⁺.

### Steps 3 to 5

Intermediates in step 3 to 5 were prepared in analogous manner according to the procedure of Example 52, steps 3-5, using (4-chloro-2-methoxyphenyl)(1H-imidazol-4-yl)methanone in place of 1H-imidazol-4-yl-[2-methoxy-4-(trifluoromethyl)phenyl]methanone in step 3, and using (R)-tetrahydro-2H-pyran-3-amine in place of (R)-1-methylpiperidin-3-amine in step 5.

### Step 6. (R)-5-Chloro-2-(4-((tetrahydro-2H-pyran-3-yl)amino)imidazo[1,5-d][1,2,4]triazin-1-yl)phenol

To a vial was added 1-(4-chloro-2-methoxy-phenyl)-N-[(3R)-tetrahydropyran-3-yl]imidazo[1,5-d][1,2,4]triazin-4-amine (56 mg, 0.16 mmol, 1 eq.) and sodium ethanethiolate (73 mg, 0.8 mmol, 5 eq.), followed by DMF (0.5 M). The reaction was heated at 135° C. for 1 hour.

Upon completion, the reaction was quenched with water and extracted with ethyl acetate. The organics were washed with brine, dried over sodium sulfate and concentrated under reduced pressure. The residue was purified by silica gel column eluting with methanol (5% ammonia hydroxide) in DCM 0-5% to give 5-chloro-2-[4-[[(3R)-tetrahydropyran-3-yl]amino]imidazo[1,5-d][1,2,4]triazin-1-yl]phenol (45 mg, 83.6% yield). MS m/z 346.7, 348.4 [M+H]⁺; ¹H NMR (400 MHz, CD₃OD) δ 9.11 (s, 1H), 8.16 (s, 1H), 7.58 (d, J=8.5 Hz, 1H), 7.08-7.01 (m, 2H), 4.21-4.10 (m, 1H), 4.08-4.00 (m, 1H), 3.81-3.73 (m, 1H), 3.51-3.37 (m, 2H), 2.19-2.10 (m, 1H), 1.89-1.74 (m, 2H), 1.74-1.59 (m, 1H). 1NH and 1 OH not observed.

### Example 54

### Preparation of Compound I-574

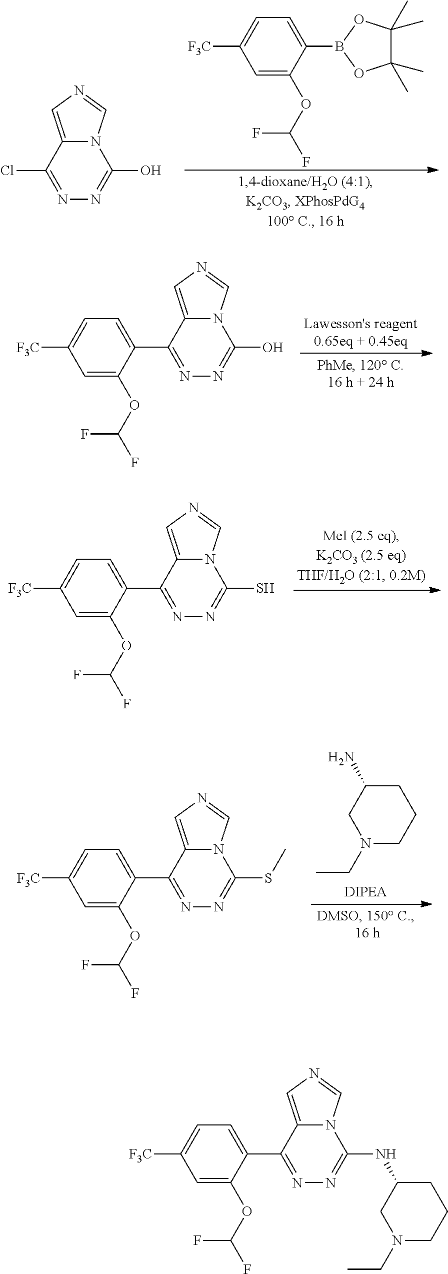

### Step 1. 1-(2-(Difluoromethoxy)-4-(trifluoromethyl)phenyl)imidazo[1,5-d][1,2,4]triazin-4-ol

To a solution of 1-chloroimidazo[1,5-d][1,2,4]triazin-4-ol (Intermediate 3, 350 mg, 2.06 mmol) in dioxane/H₂O (4 mL/1 mL) was added 2-(2-(difluoromethoxy)-4-(trifluoromethyl)phenyl)-4,4,5,5-tetramethyl-1,3,2-dioxaborolane (Intermediate 7a, 1.04 g, 3.09 mmol), XPhos Pd-G4 (124 mg, 0.144 mmol) and K₂CO₃ (852 mg, 6.18 mmol). The mixture was stirred at 100° C. for 3 h. After cooling to room temperature, the reaction was quenched with water (30 mL), extracted with EA (3×60 mL). The combined organic layers were concentrated in vacuo to get the crude product, which was purified by Pre-TLC (PE/EA=3:1) to provide the title compound as white solids (130 mg, yield 18.8%). LCMS [M+H]+=347.0.

### Step 2. 1-(2-(Difluoromethoxy)-4-(trifluoromethyl)phenyl)imidazo[1,5-d][1,2,4]triazine-4-thiol

A solution of methyl 1-(2-(difluoromethoxy)-4-(trifluoromethyl)phenyl)imidazo[1,5-d][1,2,4]triazin-4-ol (130 mg, 0.375 mmol) in Toluene (5 mL) was added Phosphorus sulfide (83 mg, 0.375 mmol). The mixture was stirred at 120° C. in a sealed tube for 24 h. After cooling to room temperature, it was concentrated in vacuo to get the crude product. The crude was purified by Pre-TLC (PE/EA=2:1) to provide the title compound as white solids (80 mg, yield 59%). MS m/z 363.1 [M+H]⁺.

### Step 3. 1-(2-(Difluoromethoxy)-4-(trifluoromethyl)phenyl)-4-(methylthio)imidazo[1,5-d][1,2,4]triazine

To a solution of 1-(2-(difluoromethoxy)-4-(trifluoromethyl)phenyl)imidazo[1,5-d][1,2,4]triazine-4-thiol (50 mg, 0.138 mmol) in THF/H₂O (4 mL/2 mL) was added Iodomethane (49 mg, 0.345 mmol) and K₂CO₃ (47 mg, 0.345 mmol). The mixture was stirred at rt for 30 mins. After that, the reaction was quenched with water (10 mL), extracted with EA (2×40 mL). The combined organic layers were concentrated in vacuo to provide the title compound which was used for next step without further purification (crude 50 mg). MS m/z 377.1 [M+H]⁺.

### Step 4. (R)-1-(2-(Difluoromethoxy)-4-(trifluoromethyl)phenyl)-N-(1-ethylpiperidin-3-yl)imidazo[1,5-d][1,2,4]triazin-4-amine

A solution of 1-(2-(difluoromethoxy)-4-(trifluoromethyl)phenyl)-4-(methylthio)imidazo[1,5-d][1,2,4]triazine (50 mg, 0.133 mmol) in DMSO (2 mL) was added (R)-1-ethylpiperidin-3-amine (43 mg, 0.332 mmol) and DIPEA (103 mg, 0.798 mmol). The mixture was stirred at 150° C. in a sealed tube for 16 h. After cooling to room temperature, it was purified by Pre-HPLC to provide the title compound as white solids (11.2 mg, yield 17.8%). MS m/z 457.2 [M+H]⁺; ¹H NMR (400 MHz, CD₃OD) δ 8.76 (s, 1H), 8.47 (s, 1H, formic acid), 7.85 (d, J=8.0 Hz, 1H), 7.75 (d, J=8.1 Hz, 1H), 7.68 (s, 1H), 7.54 (s, 1H), 6.98 (t, J=73.0 Hz, 1H), 4.61-4.41 (m, 1H), 3.79-3.54 (m, 1H), 3.29-3.24 (m, 1H), 3.11-3.00 (m, 2H), 2.95-2.72 (m, 2H), 2.27-2.07 (m, 2H), 1.96-1.77 (m, 2H), 1.32 (t, J=7.3 Hz, 3H).

The compounds below were prepared according to the procedure of Example 54 by substituting the appropriate starting materials, reagents and reaction conditions.

| Compound | Spectral Data |
|---|---|
| I-350 | MS m/z 423.05 [M + H]⁺; ¹H NMR (METHANOL-d4) δ: 8.74 (s, 1H), 7.94-8.09 |
| | (m, 2H), 7.19-7.25 (m, 1H), 7.16 (s, 1H), 4.41-4.54 (m, 1H), 3.72-3.83 (m, 3H), |
| | 3.57-3.67 (m, 1H), 2.95-3.09 (m, 2H), 2.78-2.92 (m, 2H), 2.07-2.18 (m, 1H), |
| | 1.95-2.07 (m, 1H), 1.70-1.89 (m, 2H). NH and OH not observed. |
| I-360 | MS m/z 407.4 [M + H]⁺; ¹H NMR (METHANOL-d4) δ: 8.74 (br s, 1H), 8.25 (br |
| | s, 1H, formic acid peak), 7.94-8.10 (m, 2H), 7.07-7.30 (m, 2H), 4.38-4.47 (m, |
| | 1H), 3.35-3.50 (m, 1H), 3.04 (br d, J = 9.5 Hz, 1H), 2.71-2.87 (m, 2H), 2.53-2.65 |
| | (m, 2H), 2.10 (br d, J = 10.5 Hz, 1H), 1.87-2.01 (m, 1H), 1.61-1.84 (m, 2H), 1.17 |
| | (t, J = 7.3 Hz, 3H). NH and OH not observed. |
| I-383 | MS m/z 373.8 [M + H]⁺; ¹H NMR (METHANOL-d4) δ: 8.61 (br s, 1H), 8.50 (br |
| | s, 1H, formic acid peak), 7.29-7.40 (m, 2H), 7.14 (s, 1H), 6.98 (s, 1H), |
| | 4.26-4.52 (m, 1H), 3.72 (s, 3H), 3.22-3.28 (m, 2H), 2.76-2.96 (m, 1H), |
| | 2.33-2.54 (m, 4H), 1.97-2.15 (m, 1H), 1.82-1.94 (m, 1H), 1.66-1.82 (m, 1H), |
| | 1.51-1.68 (m, 1H). NH and OH not observed. |
| I-384 | MS m/z 359.0 [M + H]⁺; ¹H NMR (METHANOL-d4) δ: 8.83 (s, 1H), 8.43 (br s, |
| | 1H, formic acid peak), 8.16 (br s, 1H), 7.96 (br d, J = 6.4 Hz, 1H), 7.01-7.11 (m, |
| | 2H), 4.40-4.56 (m, 1H), 3.38-3.47 (m, 1H), 2.94-3.11 (m, 1H), 2.58 (m, 5H), |
| | 2.08-2.24 (m, 1H), 1.94-2.05 (m, 1H), 1.80-1.91 (m, 1H), 1.64-1.78 (m, 1H). |
| | NH and OH not observed. |
| I-397 | MS m/z 380.2 [M + H]⁺; ¹H NMR (METHANOL-d4) δ: 8.77-9.01 (m, 1H), |
| | 8.08-8.25 (m, 2H), 7.22-7.39 (m, 2H), 4.28-4.47 (m, 1H), 4.12-4.26 (m, 1H), |
| | 3.82-3.99 (m, 1H), 3.43-3.67 (m, 2H), 2.19-2.35 (m, 1H), 1.78-1.95 (m, 3H). |
| | NH and OH not observed. |
| I-435 | MS m/z 345.4 [M + H]⁺; ¹H NMR (METHANOL-d4) δ: 8.85 (s, 1H), 8.15 (s, |
| | 1H), 7.89-7.99 (m, 1H), 7.05 (br s, 2H), 4.42-4.65 (m, 1H), 3.82 (m, 2H), |
| | 3.39-3.49 (m, 1H), 3.13 (m, 1H), 2.26-2.35 (m, 1H), 2.19 (m, 2H), 1.89-1.99 |
| | (m, 2H). NH and OH not observed. |
| I-436 | MS m/z 389.3 [M + H]⁺; ¹H NMR (DMSO-d6) δ: 8.84-9.03 (m, 1H), 8.11-8.27 |
| | (m, 1H), 7.95-8.05 (m, 1H), 7.08 (br s, 2H), 4.33-4.50 (m, 1H), 4.21-4.31 (m, |
| | 1H), 3.53 (br s, 2H), 3.17 (br s, 2H), 2.84 (br s, 1H), 1.94-2.21 (m, 3H), 1.77 |
| | (br d, J = 6.0 Hz, 1H), 1.53-1.67 (m, 1H), 1.35-1.52 (m, 1H). NH and OH not |
| | observed. |
| I-442 | MS m/z 371.2 [M − H]⁻; ¹H NMR (METHANOL-d4) δ: 8.69 (s, 1H), 8.34 (br s, |
| | 1H, formic acid peak), 8.02 (br s, 1H), 7.81 (br s, 1H), 6.79-7.05 (m, 2H), 4.37 |
| | (br s, 1H), 3.37 (br d, J = 8.9 Hz, 1H), 2.89-3.09 (m, 1H), 2.71 (br d, J = 5.4 Hz, |
| | 2H), 2.28-2.55 (m, 2H), 2.00-2.19 (m, 1H), 1.82-1.98 (m, 1H), 1.67-1.81 (m, |
| | 1H), 1.50-1.67 (m, 1H), 1.14 (br t, J = 7.1 Hz, 3H). NH and OH not observed. |
| I-460 | MS m/z 391.3 [M + H]⁺; ¹H NMR (METHANOL-d4) δ: 8.82 (br s, 1H), 8.44 (br |
| | s, 1H, formic acid peak), 8.15 (br s, 2H), 7.29 (br s, 2H), 5.96-6.18 (m, 2H), |
| | 4.95-5.14 (m, 1H), 3.09-3.24 (m, 2H), 2.95-3.06 (m, 1H), 2.58 (s, 3H), 2.45 (s, |
| | 1H). NH and OH not observed. |
| I-512 | MS m/z 394.3 [M + H]⁺; ¹H NMR (METHANOL-d4) δ: 8.81 (s, 1H), 7.99 (br |
| | s, 2H), 7.22 (s, 2H), 4.14-4.36 (m, 1H), 3.66-3.93 (m, 1H), 3.25-3.36 (m, 2H), |
| | 3.002-3.11 (m, 1H), 2.41-2.59 (m, 1H), 2.12-2.22 (m, 1H), 1.99-2.07 (m, 1H), |
| | 1.85-1.96 (m, 1H), 1.41-1.55 (m, 1H). NH and OH not observed. |
| I-527 | MS m/z 380.2 [M + H]⁺; ¹H NMR (METHANOL-d4) δ: 8.73-8.87 (m, 1H), |
| | 8.02-8.19 (m, 2H), 7.24-7.30 (m, 1H), 7.20 (br s, 1H), 4.14-4.37 (m, 1H), 2.67 |
| | (m, 2H), 2.26-2.33 (m, 2H), 1.46 (s, 3H). NH and OH not observed. |
| I-528 | MS m/z 411.3 [M + H]⁺; ¹H NMR (METHANOL-d4) δ: 8.69 (s, 1H), 8.44 (br |
| | s, 1H, formic acid peak), 7.99-8.10 (m, 2H), 7.16 (s, 2H), 4.84-5.05 (m, 1H), |
| | 4.61-4.70 (m, 1H), 3.24-3.32 (m, 1H), 2.95-3.10 (m, 1H), 2.34 (s, 5H), |
| | 2.14-2.27 (m, 1H), 1.68-1.97 (m, 1H). NH and OH not observed. |
| I-549 | MS m/z 346.6, 348.4 [M + H]⁺; ¹H NMR (400 MHz, CD₃OD) δ 8.82 (s, 1H), |
| | 8.15 (s, 1H), 7.95 (d, J = 8.4 Hz, 1H), 7.09-6.96 (m, 2H), 4.24 (p, J = 8.3 Hz, |
| | 1H), 2.73-2.64 (m, 2H), 2.34-2.25 (m, 2H), 1.47 (s, 3H). 2OH and 1NH are |
| | not observed. |
| I-553 | MS m/z 387.7, 389.0 [M + H]⁺; ¹H NMR (400 MHz, CD₃OD) δ 8.82 (s, 1H), |
| | 8.11 (s, 1H), 7.90 (d, J = 8.3 Hz, 1H), 7.07-7.02 (m, 2H), 4.69-4.50 (m, 1H), |
| | 3.87-3.72 (m, 1H), 3.62-3.51 (m, 1H), 3.48-3.38 (m, 1H), 3.15-2.86 (m, |
| | 2H), 2.38-2.17 (m, 2H), 2.09-1.77 (m, 2H), 1.40 (d, J = 3.6 Hz, 6H). NH and |
| | OH not observed |
| I-557 | MS m/z 377.2, 379.2 [M + H]⁺; ¹H NMR (400 MHz, CD₃OD) δ 8.78 (s, 1H), |
| | 8.21 (s, 1H, formic acid peak), 8.15 (s, 1H), 7.95 (d, J = 8.3 Hz, 1H), |
| | 7.09-7.02 (m, 2H), 5.26-4.96 (m, 1H), 4.84-4.66 (m, 1H), 3.69-3.47 (m, 1H), |
| | 2.82-2.42 (m, 7H), 2.19-1.76 (m, 1H). NH and OH not observed. |
| I-558 | MS m/z 427.3, 429.3 [M + H]⁺; ¹H NMR (400 MHz, CD₃OD) δ 8.83 (s, 1H), |
| | 8.15 (s, 1H), 7.96 (d, J = 8.5 Hz, 1H), 7.19-6.87 (m, 2H), 4.46-4.24 (m, 1H), |
| | 3.27-3.15 (m, 3H), 3.04-2.88 (m, 1H), 2.63-2.45 (m, 2H), 2.27-2.11 (m, |
| | 1H), 1.97-1.72 (m, 2H), 1.68-1.49 (m, 1H). 1NH and 1 OH not observed |
| I-559 | MS m/z 409.3, 411.3 [M + H]⁺; ¹H NMR (400 MHz, CD₃OD) δ 8.83 (s, 1H), |
| | 8.15 (s, 1H), 7.96 (d, J = 8.5 Hz, 1H), 7.06-7.01 (m, 2H), 6.04 (t, 1H), |
| | 4.49-4.20 (m, 1H), 3.04-2.74 (m, 3H), 2.56-2.34 (m, 2H), 2.29-2.07 (m, 1H), |
| | 1.96-1.67 (m, 2H), 1.66-1.49 (m, 1H). NH and OH not observed. 1H under |
| | solvent peak |
| I-561 | MS m/z 332.1, 334.1 [M + H]⁺; ¹H NMR (400 MHz, DMSO-d6) δ 13.66 (s, 1H), |
| | 8.97 (s, 1H), 8.54-8.09 (m, 2H), 8.00 (d, J = 8.5 Hz, 1H), 7.23-6.83 (m, 2H), |
| | 4.95-4.51 (m, 1H), 4.14-3.91 (m, 2H), 3.90-3.70 (m, 2H), 2.41-2.27 (m, |
| | 1H), 2.15-2.03 (m, 1H). |
| I-578 | MS m/z 360.2, 362.2 [M + H]⁺; ¹H NMR (400 MHz, CD₃OD) δ 8.83 (s, 1H), |
| | 8.16 (s, 1H), 7.97 (d, J = 8.4 Hz, 1H), 7.08-7.02 (m, 2H), 4.32-4.13 (m, 1H), |
| | 3.95-3.62 (m, 1H), 2.80-2.42 (m, 1H), 2.35-2.14 (m, 1H), 2.13-1.98 (m, |
| | 1H), 1.98-1.80 (m, 1H), 1.67-1.09 (m, 4H). 1NH and 2 OH not observed. |
| I-580 | MS m/z 330.2, 332.1 [M − H]⁻; ¹H NMR (400 MHz, CD₃OD) δ 8.87 (s, 1H), 8.16 |
| | (s, 1H), 7.97 (d, J = 8.3 Hz, 1H), 7.23-6.86 (m, 2H), 4.84-4.76 (m, 1H), |
| | 4.17-4.03 (m, 2H), 4.01-3.85 (m, 2H), 2.47 (m, 1H), 2.20 (m, 1H). 1NH and 10H |
| | not observed |
| I-581 | MS m/z 372.2, 374.2 [M − H]⁻; ¹H NMR (400 MHz, CD₃OD) δ 8.82 (s, 1H), 8.16 |
| | (s, 1H), 7.97 (d, J = 8.5 Hz, 1H), 7.33-6.71 (m, 2H), 4.20 (s, 1H), 3.47-3.41 |
| | (m, 4H), 2.66-2.53 (m, 1H), 2.29-2.10 (m, 2H), 2.01-1.89 (m, 1H), 1.70-1.17 |
| | (m, 4H). NH and OH not observed. |
| I-623 | MS m/z: 433.1, 435.1 [M + H]⁺; ¹H NMR (400 MHz, CD₃OD) δ 8.81 (s, 1H), |
| | 8.44 (s, 1H, formic acid proton), 8.10 (s, 1H), 7.84 (d, J = 8.9 Hz, 1H), |
| | 7.24-7.10 (m, 2H), 4.60-4.37 (m, 1H), 3.80 (t, J = 5.5 Hz, 2H), 3.56-3.42 (m, |
| | 1H), 3.16-3.02 (m, 1H), 2.99-2.83 (m, 2H), 2.81-2.61 (m, 2H), 2.20-2.07 (m, |
| | 1H), 2.06-1.96 (m, 1H), 1.92-1.71 (m, 2H). NH and OH not observed |
| I-624 | MS m/z: 443.2 [M + H]⁺; ¹H NMR (400 MHz, CD₃OD) δ 8.79 (s, 1H), 8.43 (s, |
| | 1H), 7.85 (d, J = 8.0 Hz, 1H), 7.75 (d, J = 8.0 Hz, 1H), 7.68 (s, 1H), 7.52 (s, |
| | 1H), 6.98 (t, J = 73.0 Hz, 1H), 4.60-4.43 (m, 1H), 3.71-3.52 (m, 1H), 3.29- |
| | 3.22 (m, 1H), 3.19-2.91 (m, 2H), 2.83 (s, 3H), 2.26-2.06 (m, 2H), 2.00- |
| | 1.75 (m, 2H). NH and OH not observed |
| I-625 | MS m/z: 461.1 [M + H]⁺; ¹H NMR (400 MHz, CD₃OD) δ 8.72 (s, 1H), 7.85 (d, J = |
| | 8.0 Hz, 1H), 7.75 (d, J = 8.4 Hz, 1H), 7.68 (s, 1H), 7.54 (s, 1H), 6.96 (t, J = |
| | 73.0 Hz, 1H), 5.12-5.07 (m, 1H), 3.58-3.48 (m, 1H), 3.37-3.32 (m, 1H), |
| | 2.85-2.70 (m, 1H), 2.65 (s, 1H), 2.61 (s, 3H), 2.59-2.45 (m, 2H), 2.07-1.88 |
| | (m, 1H). NH and OH not observed |
| I-626 | MS m/z: 371.2 [M + H]⁺; ¹H NMR (400 MHz, CD₃OD) δ 8.70 (s, 1H), 7.44 (s, |
| | 1H), 6.63 (s, 1H), 6.60 (d, J = 11.0 Hz, 1H), 4.51-4.35 (m, 1H), 3.24 (dd, J = |
| | 10.4, 5.0 Hz, 1H), 2.90-2.75 (m, 1H), 2.56 (dt, J = 7.3, 4.6 Hz, 2H), 2.34 (s, |
| | 3H), 2.33-2.21 (m, 2H), 2.14 (dd, J = 9.4, 3.7 Hz, 1H), 1.92-1.84 (m, 1H), |
| | 1.76 (ddd, J = 17.5, 12.3, 8.5 Hz, 1H), 1.63 (d, J = 10.6 Hz, 1H), 1.15 (t, J = |
| | 7.2 Hz, 3H). NH and OH not observed |
| I-627 | MS m/z: 390.2, 392.2 [M + H]⁺; ¹H NMR (400 MHz, CD₃OD) δ 8.94 (s, 1H), |
| | 8.17 (s, 1H), 7.71 (d, J = 8.6 Hz, 1H), 7.23 (m, 2H), 4.20 (p, J = 7.9 Hz, 1H), |
| | 2.70-2.63 (m, 2H), 2.32 (dd, J = 11.0, 9.1 Hz, 2H), 1.44 (s, 3H). NH and OH |
| | not observed |
| I-628 | MS m/z: 417.2, 419.2 [M + H]⁺; ¹H NMR (400 MHz, CD₃OD) δ 8.78 (s, 1H), |
| | 8.11 (s, 1H), 7.84 (d, J = 8.2 Hz, 1H), 7.18 (m, 2H), 4.58-4.45 (m, 1H), |
| | 3.81-3.66 (m, 1H), 3.45-3.34 (m, 1H), 3.07 (dd, J = 14.0, 6.9 Hz, 2H), 2.97-2.71 |
| | (m, 2H), 2.30-2.20 (m, 1H), 2.17-2.08 (m, 1H), 1.98-1.78 (m, 2H), 1.32 (t, |
| | J = 7.3 Hz, 3H). NH and OH not observed. |

### Example 55

### Preparation of Compound I-543

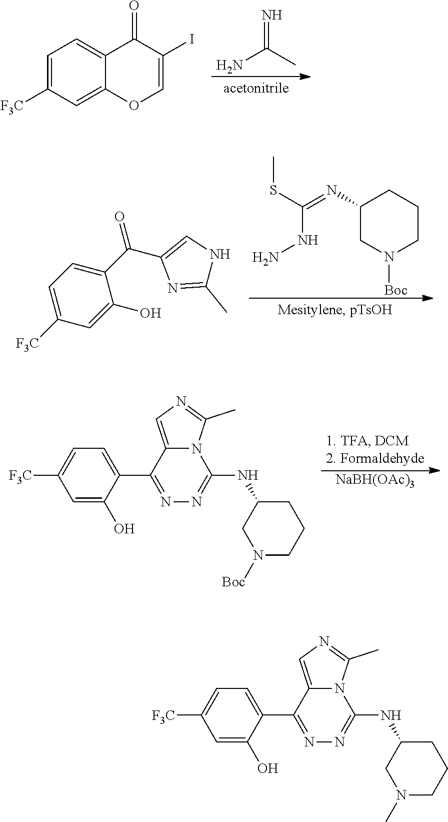

### Step 1: (2-Hydroxy-4-(trifluoromethyl)phenyl)(2-methyl-1H-imidazol-4-yl)methanone

3-Iodo-7-(trifluoromethyl)-4H-chromen-4-one (2.34 g, 6.89 mmol), acetamidine hydrochloride (0.97 g, 2.4 eq.) and potassium carbonate (2.85 g, 3 eq.) was mixed in 2-amino-3-chloro-1,4-naphthoquinone (25 mL). The resulting mixture was warmed to 80° C., and stirred for 1 h. Solvent was removed and the crude material was purified by flash column chromatography on silica gel eluting with 0-100% EtOAc in hexane to provide (2-hydroxy-4-(trifluoromethyl)phenyl)(2-methyl-1H-imidazol-4-yl)methanone (1.04 g, 55% yield). MS m/z 271.2 [M+H]⁺.

### Step 2. (R)-2-(6-Methyl-4-(piperidin-3-ylamino)imidazo[1,5-d][1,2,4]triazin-1-yl)-5-(trifluoromethyl)phenol

To a solution of (2-hydroxy-4-(trifluoromethyl)phenyl)(2-methyl-1H-imidazol-4-yl)methanone (828 mg, 3.06 mmol) in ethanol (9 mL) was added tert-butyl (3R)-3-[(E)-[hydrazino(methylsulfanyl)methylene]amino]piperidine-1-carboxylate (1.78 g, 2.1 eq.) and acetic acid (0.5 mL, 3.0 eq.). The resulting mixture was warmed to 80° C. and stirred for 1 h. The reaction mixture was partitioned between EtOAc and water. The aqueous layer was neutralized to pH ˜ 6-7, then extracted with EtOAc (×5). The organics were concentrated and the crude material was purified by flash column chromatography on silica gel eluting with 0-100% EtOAc in hexane to provide tert-butyl (R)-3-((1-(2-hydroxy-4-(trifluoromethyl)phenyl)-6-methylimidazo[1,5-d][1,2,4]triazin-4-yl)amino)piperidine-1-carboxylate (438 mg, 29% yield). MS m/z 493.2 [M+H]⁺.

### Step 3: (R)-2-(6-Methyl-4-((1-methylpiperidin-3-yl)amino)imidazo[1,5-d][1,2,4]triazin-1-yl)-5-(trifluoromethyl)phenol

Deprotection of tert-butyl (R)-3-((1-(2-hydroxy-4-(trifluoromethyl)phenyl)-6-methylimidazo[1,5-d][1,2,4]triazin-4-yl)amino)piperidine-1-carboxylate followed by reductive N-methylation according to procedure of Example 49, step 4 to provide (R)-2-(6-methyl-4-((1-methylpiperidin-3-yl)amino)imidazo[1,5-d][1,2,4]triazin-1-yl)-5-(trifluoromethyl)phenol: MS m z: 407.1 [M+H]^{m}; ¹H NMR (400 MHz, CD₃OD) δ 8.53 (s, 1H, formic acid proton), 7.89 (s, 1H), 7.64 (s, 1H), 7.26 (d, J=8.5 Hz, 1H), 7.23 (s, 1H), 4.42-3.88 (m, 1H), 3.15-3.05 (m, 1H), 3.02 (s, 3H), 2.86-2.56 (m, 3H), 2.51 (s, 3H), 2.04-1.90 (m, 2H), 1.85-1.61 (m, 2H). NH and OH not observed.

### Example 56

### Preparation of Compounds I-386 and I-398

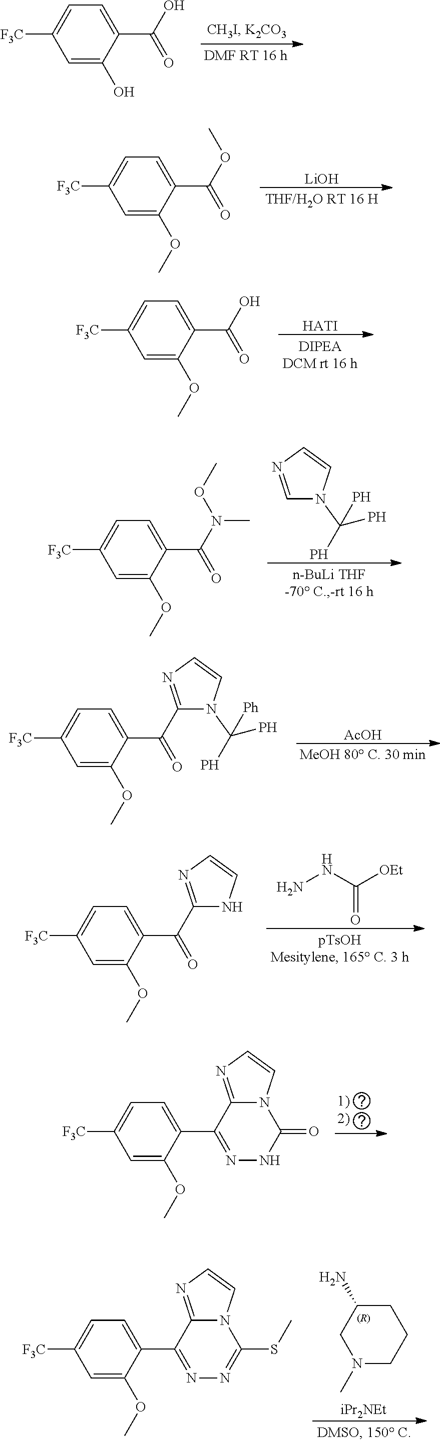

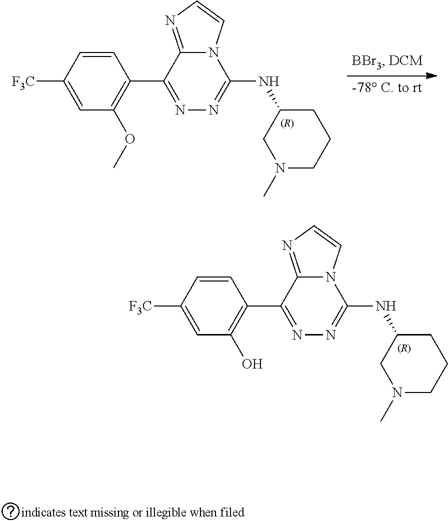

### Step 1. Methyl 2-methoxy-4-(trifluoromethyl)benzoate

To a solution of 2-hydroxy-4-(trifluoromethyl) benzoic acid (32.0 g, 155 mmol, 1.0 eq.) and K₂CO₃ (64.0 g, 465 mmol, 3.0 eq.) in DMF (320 mL, 0.5 M) was added iodomethane (55.0 g, 387 mmol, 2.5 eq.) at 0° C. After stirring at 0° C. for 1 h, the reaction mixture was warmed to room temperature and stirred for 16 h. The reaction mixture was diluted with water and extracted with EA. The organic layer was washed with brine, dried over anhydrous Na₂SO₄ and concentrated. The crude product was purified by flash column chromatography (ethyl acetate/hexane=0%˜30%) to obtain methyl 2-methoxy-4-(trifluoromethyl) benzoate (36.0 g, 154 mmol, 99% yield) as colorless oil. ¹H NMR (400 MHz, Chloroform-d) δ: 7.85 (d, J=8.2 Hz, 1H), 7.23 (d, J=8.2 Hz, 1H), 7.20 (s, 1H), 3.95 (s, 3H), 3.91 (s, 3H).

### Step 2. 2-Methoxy-4-(trifluoromethyl)benzoic acid

To a solution of methyl 2-methoxy-4-(trifluoromethyl) benzoate (36.0 g, 153 mmol, 1.0 eq.) in tetrahydrofuran and methanol (300 mL, 1:1) was added lithium hydroxide (9.60 g, 229 mmol, 1.5 eq.) at room temperature. The reaction mixture was stirred at room temperature for 16 hours and then the solvent was removed. The residue was dissolved in water, acidified with hydrochloric acid (1M) and extracted with EA (200 mL×3). The combined organic layer was dried over anhydrous sodium sulfate and concentrated under reduced pressure to obtain 2-methoxy-4-(trifluoromethyl) benzoic acid (28.4 g, 129 mmol, 84.3% yield) as a white solid, which was used to the next step without purification. MS m/z 221.1 [M+H]⁺.

### Step 3. N,2-Dimethoxy-N-methyl-4-(trifluoromethyl)benzamide

To a solution of 2-methoxy-4-(trifluoromethyl) benzoic acid (28.4 g, 129 mmol, 1.0 eq.) in DCM (300 mL, 0.4 M) were added HATU (58.9 g, 155 mmol, 1.2 eq.), DIPEA (50.0 g, 387 mmol, 3.0 eq.) and N,O-dimethylhydroxylamine hydrochloride (15.0 g, 155 mmol, 1.2 eq.). The reaction mixture was stirred at room temperature for 16 h. Upon completion, the mixture was diluted with water (200 mL) and extracted with EtOAc (2×400 mL). The organic layer was washed with brine, dried over Na₂SO₄ and concentrated. The crude product was purified by silica gel column chromatography (ethyl acetate/hexane=0%˜30%) to obtain N,2-dimethoxy-N-methyl-4-(trifluoromethyl) benzamide (33.7 g, 128.1 mmol, yield 99%) as a colorless oil. MS m/z 264.1 [M+H]⁺, ¹H NMR (400 MHz, DMSO-d6) δ: 7.49 (d, J=7.8 Hz, 1H), 7.39 (s, 1H), 7.36 (d, J=7.8 Hz, 1H), 3.90 (s, 3H), 3.45 (s, 3H), 3.27 (s, 3H).

### Step 4. (2-Methoxy-4-(trifluoromethyl) phenyl) (1-trityl-1H-imidazol-2-yl)methanone

To a solution of 1-trityl-1H-imidazole (3.72 g, 12.0 mmol, 1.0 eq.) in THE (60 mL) was added n-BuLi (7.5 mL, 1.6 M in THF, 12.0 mmol, 1.0 eq.) at −78° C. The reaction mixture was stirred at −78° C. for 1 h, then N,2-dimethoxy-N-methyl-4-(trifluoromethyl) benzamide (2.63 g, 10.0 mmol, 0.8 eq.) was added. The reaction mixture warmed to room temperature and stirred for 16 h. Upon completion, the mixture was quenched with water (50 mL) and extracted with EtOAc (50 mL×3). The organic layer was washed with brine, dried over Na₂SO₄ and concentrated. The crude product was purified by silica gel column chromatography (ethyl acetate/hexane=0% 30%) to obtain (2-methoxy-4-(trifluoromethyl) phenyl) (1-trityl-1H-imidazol-2-yl)methanone (374 mg, yield 6.1%) as a white solid. ¹H NMR (400 MHz, Chloroform-d) δ: 7.33-7.27 (m, 9H), 7.22-7.10 (m, 10H), 7.06 (s, 1H), 3.75 (s, 3H).

### Step 5. (1H-Imidazol-2-yl) (2-methoxy-4-(trifluoromethyl)phenyl)methanone

A mixture of (2-methoxy-4-(trifluoromethyl) phenyl) (1-trityl-1H-imidazol-2-yl)methanone (1.88 g, 3.60 mmol, 1.0 eq.) in 5% acetic acid/methanol solution (126 mL) was heated to reflux for 0.5 h. After cooling to room temperature, the mixture was concentrated under reduced pressure. The residue was purified by silica gel column chromatography (ethyl acetate/hexane=0%˜30%) to obtain (1H-imidazol-2-yl) (2-methoxy-4-(trifluoromethyl) phenyl)methanone (825 mg, yield 84.8%) as a white solid. MS m/z 271.1 [M+H]⁺, ¹H NMR (400 MHz, Chloroform-d) δ: 10.65 (s, 1H), 7.79 (d, J=7.8 Hz, 1H), 7.35-7.32 (d, J=9.2 Hz, 2H), 7.29 (d, J=1.4 Hz, 1H), 7.22 (s, 1H), 3.88 (s, 3H).

### Step 6. 8-(2-Methoxy-4-(trifluoromethyl) phenyl) imidazo[1,2-d][1,2,4] triazin-5(6H)-one

To a solution of (1H-imidazol-2-yl)(2-methoxy-4-(trifluoromethyl)phenyl)methanone (825 mg, 3.00 mmol, 1.0 eq.) in 1,3,5-trimethyl-benzene (80 mL, 0.04 M) was added toluene-4-sulfonic acid (58 mg, 0.300 mmol, 0.1 eq.) and ethyl hydrazinecarboxylate (476 mg, 4.50 mmol, 1.5 eq.). After stirring at 165° C. for 3 h, the mixture was cooled to room temperature and diluted with EA (150 mL). The organic phase was washed with water and brine, dried over Na₂SO₄ and concentrated. The crude product was purified by silica gel column chromatography (ethyl acetate/hexane=0%˜30%) to obtain 8-(2-methoxy-4-(trifluoromethyl) phenyl) imidazo[1,2-d][1,2,4] triazin-5(6H)-one (295 mg, yield 31.7%) as a white solid. MS m/z 311.0 [M+H]⁺, ¹H NMR (400 MHz, DMSO-d6) δ: 13.13 (s, 1H), 8.11 (d, J=1.4 Hz, 1H), 7.66 (d, J=7.8 Hz, 1H), 7.62 (s, 1H), 7.48 (s, 1H), 7.46 (d, J=7.8 Hz, 1H), 3.82 (s, 3H).

### Step 7. 8-[2-Methoxy-4-(trifluoromethyl)phenyl]-6H-imidazo[1,2-d][1,2,4]triazine-5-thione

A suspension of 8-[2-methoxy-4-(trifluoromethyl)phenyl]-6H-imidazo[1,2-d][1,2,4]triazin-5-one (0.195 g, 0.629 mmol) in PhMe (2.5 mL) was added Lawesson's reagent (0.183 g, 0.440 mmol). The mixture was heated to 120° C. The reaction cooled to rt and diluted with EtOAc. The solution was washed with water, sat. NaHCO₃, brine, dried (Na₂SO₄), filtered and concentrated. Purification by chromatography on SiO₂ (EtOAc:DCM, 0 to 20%) gave a white solid (0.117 g, 57%). MS m/z 327.3 [M+H]⁺.

### Step 8. 8-[2-Methoxy-4-(trifluoromethyl)phenyl]-5-methylsulfanyl-imidazo[1,2-d][1,2,4]triazine

A solution of 8-[2-methoxy-4-(trifluoromethyl)phenyl]-6H-imidazo[1,2-d][1,2,4]triazine-5-thione (0.189 g, 0.579 mmol) in THF/H₂O (2:1, 1.5 mL) was added MeI (65 mL, 1.04 mmol) and K₂CO₃ (0.144 g, 1.04 mmol). The mixture was stirred at rt for 40 min. The reaction was diluted with EtOAc, washed with brine, dried (Na₂SO₄), filtered and concentrated. Purification by chromatography on SiO₂ (EtOAc:hexanes, 5-60%) gave a white solid (0.158 g, 80%). MS m/z 341.3 [M+H]⁺; H NMR (400 MHz, CD₃OD) δ 8.02 (s, 1H), 7.87 (s, 1H), 7.71 (d, J=7.40 Hz, 1H), 7.50-7.42 (m, 2H), 3.84 (s, 3H), 2.96 (s, 3H).

### Step 9. 8-[2-Methoxy-4-(trifluoromethyl)phenyl]-N-[(3R)-1-methyl-3-piperidyl]imidazo[1,2-d][1,2,4]triazin-5-amine formic acid salt

A mixture of 8-[2-methoxy-4-(trifluoromethyl)phenyl]-5-methylsulfanyl-imidazo[1,2-d][1,2,4]triazine (0.095 g, 0.280 mmol) and (3R)-1-methylpiperidin-3-amine (0.080 g, 0.700 mmol) in DMSO (0.37 mL) and iPr₂NEt (0.15 mL, 0.840 mmol) was heated to 150° C. for 20 h. The reaction was cooled to rt and diluted with DCM/iPrOH (9:1). The solution was washed with brine, dried (Na₂SO₄), filtered and concentrated. Purification by reverse phase chromatography (0.1% formic acid in MeCN:0.1% formic acid in H₂O, 5 to 100%) gave a tan solid (0.069 g, 55%). MS m/z 407.1 [M+H]⁺; ¹H NMR (500 MHz, CD₃OD) δ 8.45 (s, 1H, formic acid), 8.14 (s, 1H), 7.75 (s, 1H), 7.63 (d, J=8.09 Hz, 1H), 7.45-7.40 (m, 2H), 4.61-4.49 (m, 1H), 3.82 (s, 3H), 3.64-3.49 (m, 1H), 3.20-3.10 (m, 1H), 2.76-2.63 (m, 5H), 2.29-2.14 (m, 1H), 2.11-2.00 (m, 1H), 1.95-1.71 (m, 2H). NH not observed.

### Step 10. 2-[5-[[(3R)-1-Methyl-3-piperidyl]amino]imidazo[1,2-d][1,2,4]triazin-8-yl]-5-(trifluoromethyl)phenol formic acid salt

A solution of 8-[2-methoxy-4-(trifluoromethyl)phenyl]-N-[(3R)-1-methyl-3-piperidyl]-imidazo[1,2-d][1,2,4]triazin-5-amine (0.087 g, 0.21 mmol) in DCM (0.80 mL) was cooled to −78° C. BBr₃ (1.0 M in DCM, 2.1 mL, 2.1 mmol) was added and the reaction stirred at −78° C. for 20 min before warming to rt. After 1.5 h, the reaction was quenched by slowly adding the reaction mixture to a stirred solution of DCM/MeOH (10:1, 11 mL). Sat. NaHCO₃ was then added, and the mixture was stirred for 15 min. The layers were separated, and the aqueous phase was extracted with DCM. The combined organic extracts were washed with brine, dried (Na₂SO₄), filtered, and concentrated. Purification by chromatography on SiO₂ (MeOH:DCM, 0 to 10%) followed by reverse phase chromatography (0.1% formic acid in MeCN:0.1% formic acid in H₂O, 5 to 100%) gave a tan solid (0.040 g, 43%). MS m/z 393.4 [M+H]⁺; ¹H NMR (500 MHz, DMSO-d₆) δ 9.50 (d, J=8.4 Hz, 1H), 8.44 (s, 1H), 8.20-8.00 (m, 2H, including formic acid), 7.96 (s, 1H), 7.35 (br d, J=8.5 Hz, 1H), 7.28 (s, 1H), 4.36-4.27 (m, 1H), 3.08 (d, J=8.1 Hz, 1H), 2.73 (br d, J=11.1 Hz, 1H), 2.24 (s, 3H), 2.08-1.92 (m, 3H), 1.84-1.73 (m, 1H), 1.67-1.55 (m, 1H), 1.51-1.40 (m, 1H). 1H not observed (OH).

### Example 57

### Preparation of Compound I-461

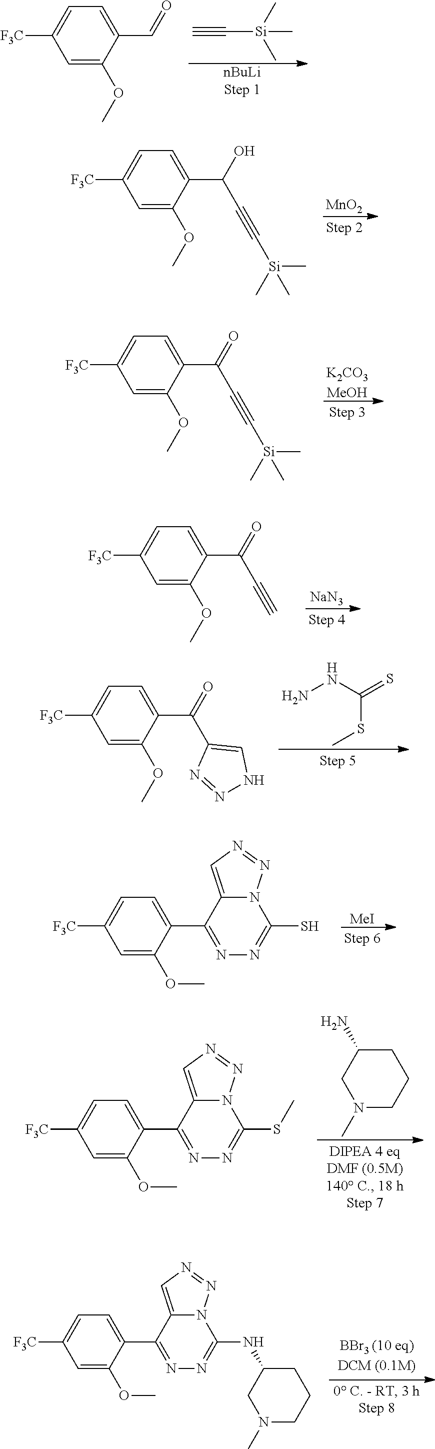

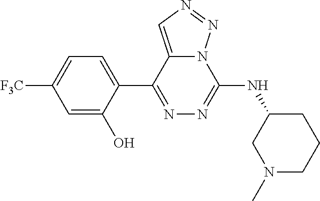 [SMILES: CN1CCC[C@@H](Nc2nnc(-c3ccc(C(F)(F)F)cc3O)c3cnnn23)C1]

### Step 1: 1-[2-Methoxy-4-(trifluoromethyl) phenyl]-3-trimethylsilyl-prop-2-yn-1-ol

To a solution of ethynyl(trimethyl)silane (2.65 g, 1.1 eq., 27 mmol) in tetrahydrofuran (0.25 M) was added n-butyllithium (in hexanes, 1.05 eq., 26 mmol, 2.5 M) dropwise at −78° C. The resulting mixture was stirred at −78° C. for 1 hour, followed by addition of a solution of 2-methoxy-4-(trifluoromethyl) benzaldehyde (5.0 g, 24 mmol) in THE (5 mL) dropwise. The reaction was then warmed to 0° C. After stirring at 0° C. for 20 minutes, ammonium chloride solution was added and the reaction was diluted with EtOAc. The organics were washed with water, sat. NaHCO₃, brine, dried (Na₂SO₄), filtered and concentrated. The crude material was purified by flash column chromatography on silica gel eluting with 0-50% EtOAc in hexane to provide 1-[2-methoxy-4-(trifluoromethyl) phenyl]-3-trimethylsilyl-prop-2-yn-1-ol (6.7 g, 90% Yield).

### Step 2. 1-[2-Methoxy-4-(trifluoromethyl) phenyl]-3-trimethylsilyl-prop-2-yn-1-one

A mixture of 1-[2-methoxy-4-(trifluoromethyl) phenyl]-3-trimethylsilyl-prop-2-yn-1-ol (6.7 g, 22 mmol) and manganese dioxide (5 eq., 110 mmol) in dichloromethane (0.2 M) was stirred at room temperature for 4 h. The crude material was passed through a celite pad and washed with DCM. The organic volatiles were removed, and the crude material was used for the next step without purification.

### Step 3: 1-[2-Methoxy-4-(trifluoromethyl) phenyl]-3-H-prop-2-yn-1-one

A mixture of 1-[2-methoxy-4-(trifluoromethyl) phenyl]-3-trimethylsilyl-prop-2-yn-1-one (6.7 g, 22 mmol) and potassium carbonate (5 eq., 110 mmol) in methanol (0.2 M) was stirred at room temperature for 20 minutes. The crude material was passed through a celite pad and the organic volatiles were removed. The crude material was concentrated down and loaded on the column for purification eluting with hexane/EtOAc (0-50%) to afford 1-[2-methoxy-4-(trifluoromethyl) phenyl]prop-2-yn-1-one (2.1 g, 41% Yield).

### Step 4. [2-Methoxy-4-(trifluoromethyl) phenyl]-(1H-triazol-4-yl)methanone

A suspension of sodium azide (4 eq., 2.4 g, 37 mmol) in DMSO (0.3 M, 31 mL) was heated to 90° C. A solution of 1-[2-methoxy-4-(trifluoromethyl) phenyl]prop-2-yn-1-one (750 mg, 0.75 g, 3.28 mmol) in DMSO (10 ml) was added dropwise. The resulting mixture was continued to stir at 90° C. for 4 h. After cooling, the reaction was poured into ice/HCl (aq.), then extracted with EtOAc. The organic phase was dried over MgSO₄, filtered, and concentrated in vacuo. The crude material was purified by flash column chromatography on silica gel eluting with 0-100% EtOAc in hexane to provide [2-methoxy-4-(trifluoromethyl) phenyl]-(1H-triazol-4-yl)methanone (460 mg, 18% Yield). MS m/z 272.0 [M+H]⁺

### Step 5-8: (R)-2-(7-((1-Methylpiperidin-3-yl)amino)-[1,2,3]triazolo[1,5-d][1,2,4]triazin-4-yl)-5-(trifluoromethyl)phenol

The title compound was prepared, as formic acid salt, in analogous manner according to the procedure of Example 52, steps 3-6, using [2-methoxy-4-(trifluoromethyl) phenyl]-(1H-triazol-4-yl)methanone in place of 1H-imidazol-4-yl-[2-methoxy-4-(trifluoromethyl)phenyl]methanone in step 3. MS m/z 394.3 [M+H]⁺; ¹H NMR (CD₃OD) δ: 8.81 (s, 1H), 8.47 (br s, 1H, formic acid peak), 8.07-8.19 (m, 1H), 7.32 (br s, 2H), 4.57-4.70 (m, 1H), 2.88-3.03 (m, 1H), 2.68 (s, 1H), 2.52 (s, 5H), 2.07-2.27 (m, 1H), 1.97 (br s, 1H), 1.72-1.91 (m, 2H), NH and OH not observed.

### Biological Assays

### IL-1β Secretion Assay:

Monocytic THP-1 cells (ATCC: TIB-202) were maintained in growth media consisting of RPMI 1640 medium (ThermoFisher, Cat #11875-085), 10% FBS (ThermoFisher) and 0.05 mM β-mercaptoethanol (ThermoFisher, Cat #21985-023), according to the provider's instructions. The cell concentration was adjusted to 7.5×10⁵ cells/mL, and plated in complete growth media with a final concentration of 100 ng/mL phorbol 12-myristate 13-acetate (PMA, Sigma #P8139). Cells were seeded at 100 μL/well into a 96-well cell culture plate (ThermoFisher Cat #165305) and allowed to differentiate for 24 h in a cell culture incubator at 37° C. with 5% CO₂. Cells were washed 1× with 100 ul PBS and replaced with fresh RPMI+5% FBS. Compounds were serial diluted in DMSO with 3 fold dilution for a total of 7 concentrations. Diluted compounds were added to the cells at a ratio of 1:200 and incubated for 20 h. The NLRP3 inflammasome was activated with the addition of 2.5 μM Nigericin (Sigma: Cat#SML 1779-1 ml), for 3 h. After incubation, 15 μL of conditioned media was removed and assayed for levels of IL-1β using the HTRF IL-1β assay kit (Cisbio: Cat #62HIL1BPEH) as per the manufacturer's instructions.

Compounds, once produced and prepared according to the present invention, can be assessed in variety of assays to characterize their activities. For example, NLRP3-dependent IL1β secretion was evaluated in THP1 cells. IC50 values of IL1β inhibition were calculated from the plot of percentage of inhibition versus the inhibitor concentration by a logistics fit. TABLE I depict examples of compounds according to generic Formulae I. Data which is <1 nM is listed as *****; data 1-10 nM is listed as ****; data 10-100 nM is listed as ***, data 100-300 nM is listed as **, data >300 nM is listed as *. The data obtained from the THP1 NLRP3-dependent IL-1β secretion assay demonstrate that the compounds of the present invention could be used to treat diseases mediated through NLRP3 activation.

TABLE I

| | | IC₅₀ |
|---|---|---|
| Compound | | IL-1β |
| Number | Structure | Inhibition |
| I-1 | 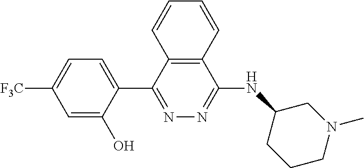 [SMILES: CN1CCC[C@@H](Nc2nnc(-c3ccc(C(F)(F)F)cc3O)c3ccccc23)C1] | ***** |
| I-2 | 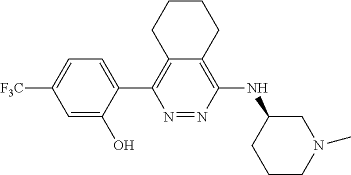 [SMILES: CN1CCC[C@@H](Nc2nnc(-c3ccc(C(F)(F)F)cc3O)c3c2CCCC3)C1] | ***** |
| I-3 | 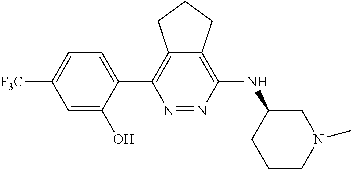 [SMILES: CN1CCC[C@@H](Nc2nnc(-c3ccc(C(F)(F)F)cc3O)c3c2CCC3)C1] | **** |
| I-4 | 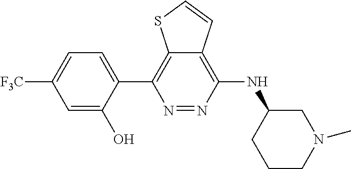 [SMILES: CN1CCC[C@@H](Nc2nnc(-c3ccc(C(F)(F)F)cc3O)c3sccc23)C1] | **** |
| I-5 | 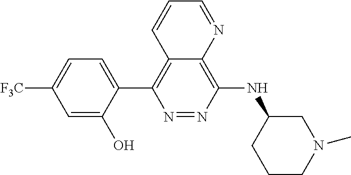 [SMILES: CN1CCC[C@@H](Nc2nnc(-c3ccc(C(F)(F)F)cc3O)c3cccnc23)C1] | ***** |
| I-6 | 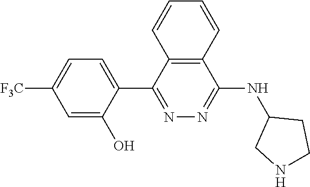 [SMILES: Oc1cc(C(F)(F)F)ccc1-c1nnc(NC2CCNC2)c2ccccc12] | *** |
| I-7 | 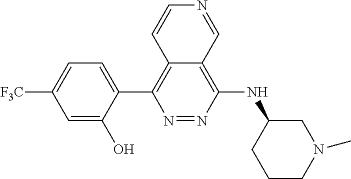 [SMILES: CN1CCC[C@@H](Nc2nnc(-c3ccc(C(F)(F)F)cc3O)c3ccncc23)C1] | ***** |
| I-8 | 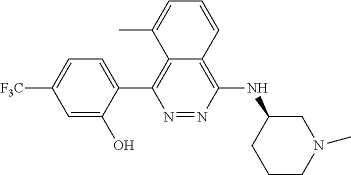 [SMILES: Cc1cccc2c(N[C@@H]3CCCN(C)C3)nnc(-c3ccc(C(F)(F)F)cc3O)c12] | **** |
| I-9 | 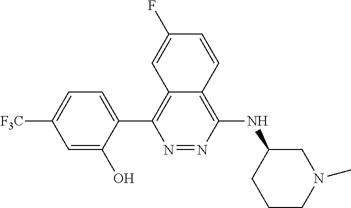 [SMILES: CN1CCC[C@@H](Nc2nnc(-c3ccc(C(F)(F)F)cc3O)c3cc(F)ccc23)C1] | **** |
| I-10 | 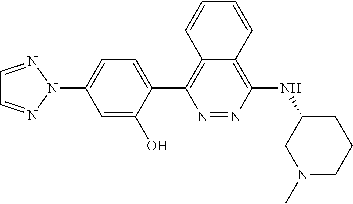 [SMILES: CN1CCC[C@@H](Nc2nnc(-c3ccc(-n4nccn4)cc3O)c3ccccc23)C1] | **** |
| I-11 | 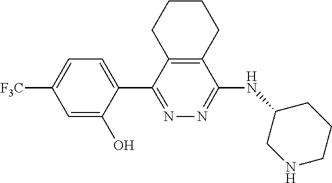 [SMILES: Oc1cc(C(F)(F)F)ccc1-c1nnc(N[C@@H]2CCCNC2)c2c1CCCC2] | *** |
| I-12 | 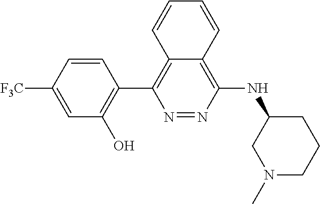 [SMILES: CN1CCC[C@H](Nc2nnc(-c3ccc(C(F)(F)F)cc3O)c3ccccc23)C1] | ** |
| I-13 | 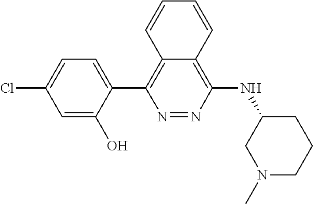 [SMILES: CN1CCC[C@@H](Nc2nnc(-c3ccc(Cl)cc3O)c3ccccc23)C1] | ***** |
| I-14 | 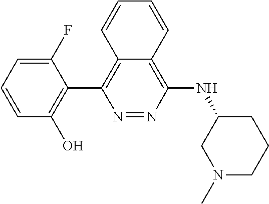 [SMILES: CN1CCC[C@@H](Nc2nnc(-c3c(O)cccc3F)c3ccccc23)C1] | **** |
| I-15 | 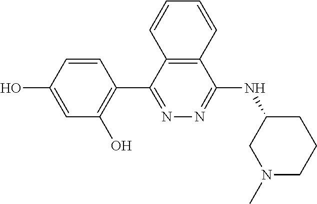 [SMILES: CN1CCC[C@@H](Nc2nnc(-c3ccc(O)cc3O)c3ccccc23)C1] | ** |
| I-16 | 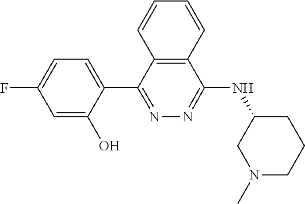 [SMILES: CN1CCC[C@@H](Nc2nnc(-c3ccc(F)cc3O)c3ccccc23)C1] | **** |
| I-17 | 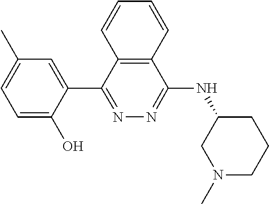 [SMILES: Cc1ccc(O)c(-c2nnc(N[C@@H]3CCCN(C)C3)c3ccccc23)c1] | ** |
| I-18 | 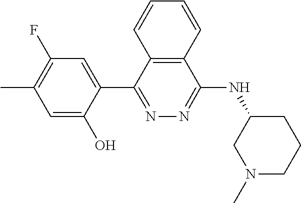 [SMILES: Cc1cc(O)c(-c2nnc(N[C@@H]3CCCN(C)C3)c3ccccc23)cc1F] | **** |
| I-19 | 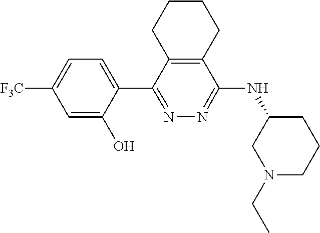 [SMILES: CCN1CCC[C@@H](Nc2nnc(-c3ccc(C(F)(F)F)cc3O)c3c2CCCC3)C1] | ***** |
| I-20 | 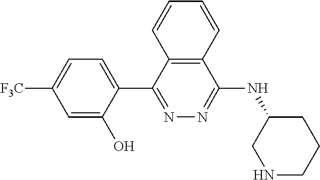 [SMILES: Oc1cc(C(F)(F)F)ccc1-c1nnc(N[C@@H]2CCCNC2)c2ccccc12] | ***** |
| I-21 | 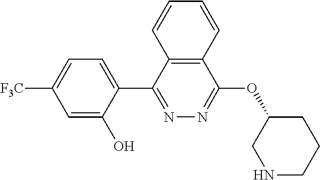 [SMILES: Oc1cc(C(F)(F)F)ccc1-c1nnc(O[C@@H]2CCCNC2)c2ccccc12] | *** |
| I-22 | 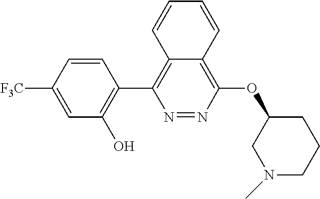 [SMILES: CN1CCC[C@H](Oc2nnc(-c3ccc(C(F)(F)F)cc3O)c3ccccc23)C1] | ** |
| I-23 | 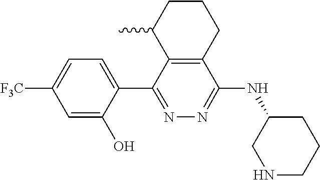 [SMILES: CC1CCCc2c(N[C@@H]3CCCNC3)nnc(-c3ccc(C(F)(F)F)cc3O)c21] | *** |
| I-24 | 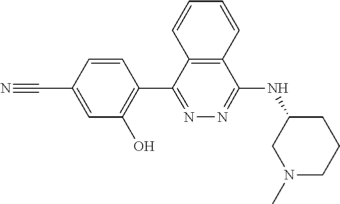 [SMILES: CN1CCC[C@@H](Nc2nnc(-c3ccc(C#N)cc3O)c3ccccc23)C1] | ***** |
| I-25 | 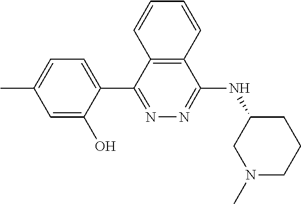 [SMILES: Cc1ccc(-c2nnc(N[C@@H]3CCCN(C)C3)c3ccccc23)c(O)c1] | ***** |
| I-26 | 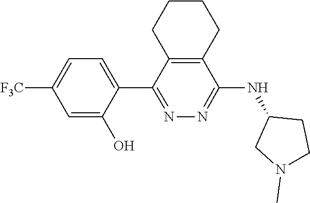 [SMILES: CN1CC[C@@H](Nc2nnc(-c3ccc(C(F)(F)F)cc3O)c3c2CCCC3)C1] | *** |
| I-27 | 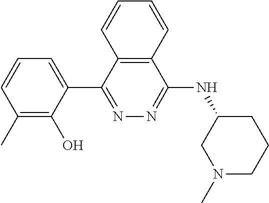 [SMILES: Cc1cccc(-c2nnc(N[C@@H]3CCCN(C)C3)c3ccccc23)c1O] | ***** |
| I-28 | 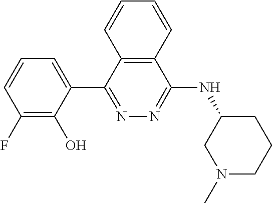 [SMILES: CN1CCC[C@@H](Nc2nnc(-c3cccc(F)c3O)c3ccccc23)C1] | *** |
| I-29 | 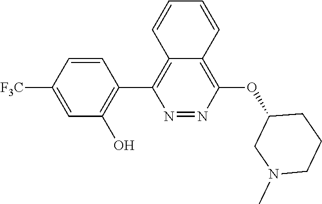 [SMILES: CN1CCC[C@@H](Oc2nnc(-c3ccc(C(F)(F)F)cc3O)c3ccccc23)C1] | **** |
| I-30 | 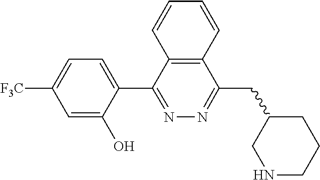 [SMILES: Oc1cc(C(F)(F)F)ccc1-c1nnc(CC2CCCNC2)c2ccccc12] | *** |
| I-31 | 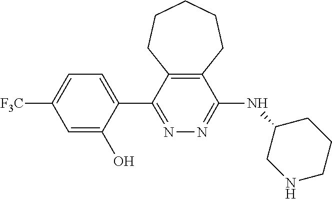 [SMILES: Oc1cc(C(F)(F)F)ccc1-c1nnc(N[C@@H]2CCCNC2)c2c1CCCCC2] | ***** |
| I-32 | 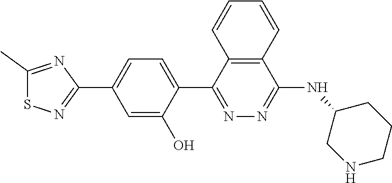 [SMILES: Cc1nc(-c2ccc(-c3nnc(N[C@@H]4CCCNC4)c4ccccc34)c(O)c2)ns1] | *** |
| I-33 | 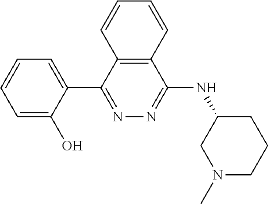 [SMILES: CN1CCC[C@@H](Nc2nnc(-c3ccccc3O)c3ccccc23)C1] | **** |
| I-34 | 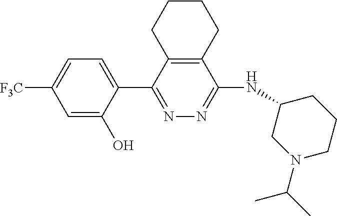 [SMILES: CC(C)N1CCC[C@@H](Nc2nnc(-c3ccc(C(F)(F)F)cc3O)c3c2CCCC3)C1] | ***** |
| I-35 | 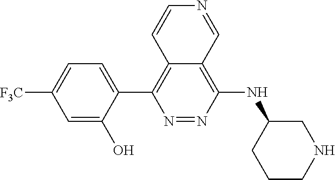 [SMILES: Oc1cc(C(F)(F)F)ccc1-c1nnc(N[C@@H]2CCCNC2)c2cnccc12] | ***** |
| I-36 | 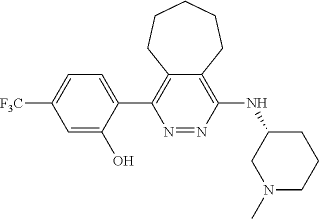 [SMILES: CN1CCC[C@@H](Nc2nnc(-c3ccc(C(F)(F)F)cc3O)c3c2CCCCC3)C1] | ***** |
| I-37 | 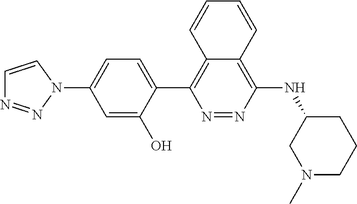 [SMILES: CN1CCC[C@@H](Nc2nnc(-c3ccc(-n4ccnn4)cc3O)c3ccccc23)C1] | *** |
| I-38 | 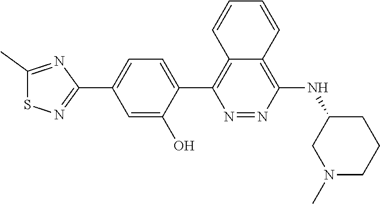 [SMILES: Cc1nc(-c2ccc(-c3nnc(N[C@@H]4CCCN(C)C4)c4ccccc34)c(O)c2)ns1] | ***** |
| I-39 | 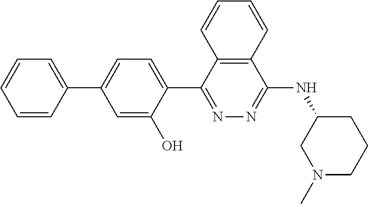 [SMILES: CN1CCC[C@@H](Nc2nnc(-c3ccc(-c4ccccc4)cc3O)c3ccccc23)C1] | **** |
| I-40 | 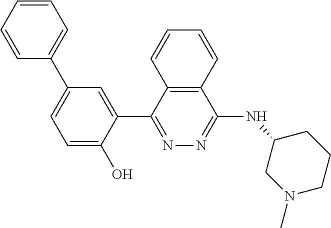 [SMILES: CN1CCC[C@@H](Nc2nnc(-c3cc(-c4ccccc4)ccc3O)c3ccccc23)C1] | ** |
| I-41 | 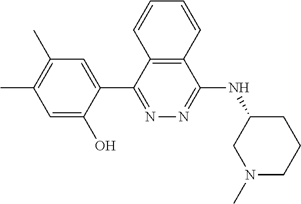 [SMILES: Cc1cc(O)c(-c2nnc(N[C@@H]3CCCN(C)C3)c3ccccc23)cc1C] | **** |
| I-42 | 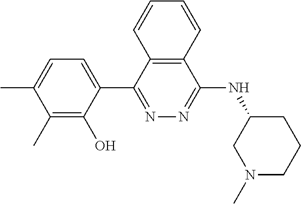 [SMILES: Cc1ccc(-c2nnc(N[C@@H]3CCCN(C)C3)c3ccccc23)c(O)c1C] | ***** |
| I-43 | 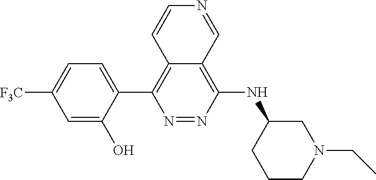 [SMILES: CCN1CCC[C@@H](Nc2nnc(-c3ccc(C(F)(F)F)cc3O)c3ccncc23)C1] | ***** |
| I-44 | 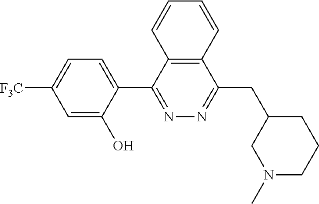 [SMILES: CN1CCCC(Cc2nnc(-c3ccc(C(F)(F)F)cc3O)c3ccccc23)C1] | **** |
| I-45 | 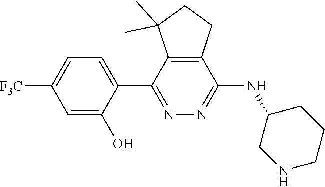 [SMILES: CC1(C)CCc2c(N[C@@H]3CCCNC3)nnc(-c3ccc(C(F)(F)F)cc3O)c21] | **** |
| I-46 | 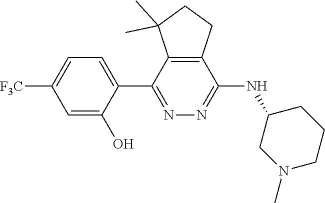 [SMILES: CN1CCC[C@@H](Nc2nnc(-c3ccc(C(F)(F)F)cc3O)c3c2CCC3(C)C)C1] | *** |
| I-47 | 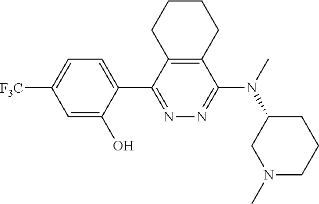 [SMILES: CN1CCC[C@@H](N(C)c2nnc(-c3ccc(C(F)(F)F)cc3O)c3c2CCCC3)C1] | ***** |
| I-48 | 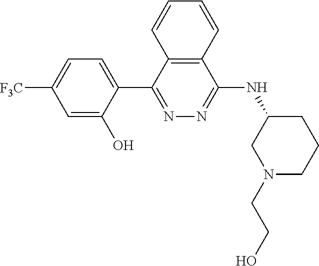 [SMILES: OCCN1CCC[C@@H](Nc2nnc(-c3ccc(C(F)(F)F)cc3O)c3ccccc23)C1] | ***** |
| I-49 | 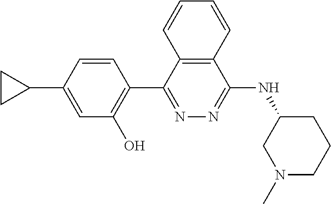 [SMILES: CN1CCC[C@@H](Nc2nnc(-c3ccc(C4CC4)cc3O)c3ccccc23)C1] | ***** |
| I-50 | 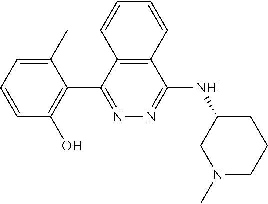 [SMILES: Cc1cccc(O)c1-c1nnc(N[C@@H]2CCCN(C)C2)c2ccccc12] | ***** |
| I-51 | 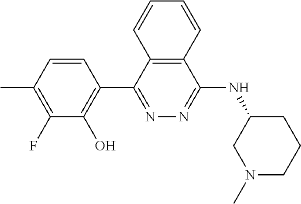 [SMILES: Cc1ccc(-c2nnc(N[C@@H]3CCCN(C)C3)c3ccccc23)c(O)c1F] | ***** |
| I-52 | 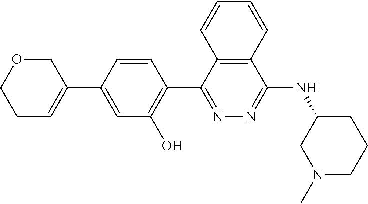 [SMILES: CN1CCC[C@@H](Nc2nnc(-c3ccc(C4=CCCOC4)cc3O)c3ccccc23)C1] | **** |
| I-53 | 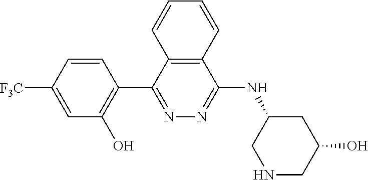 [SMILES: Oc1cc(C(F)(F)F)ccc1-c1nnc(N[C@H]2CNC[C@@H](O)C2)c2ccccc12] | **** |
| I-54 | 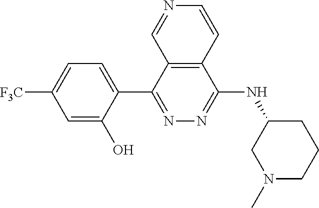 [SMILES: CN1CCC[C@@H](Nc2nnc(-c3ccc(C(F)(F)F)cc3O)c3cnccc23)C1] | ***** |
| I-55 | 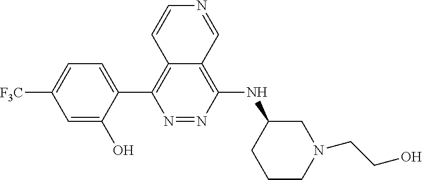 [SMILES: OCCN1CCC[C@@H](Nc2nnc(-c3ccc(C(F)(F)F)cc3O)c3ccncc23)C1] | ***** |
| I-56 | 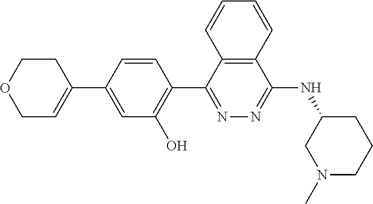 [SMILES: CN1CCC[C@@H](Nc2nnc(-c3ccc(C4=CCOCC4)cc3O)c3ccccc23)C1] | *** |
| I-57 | 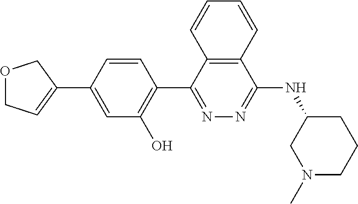 [SMILES: CN1CCC[C@@H](Nc2nnc(-c3ccc(C4=CCOC4)cc3O)c3ccccc23)C1] | **** |
| I-58 | 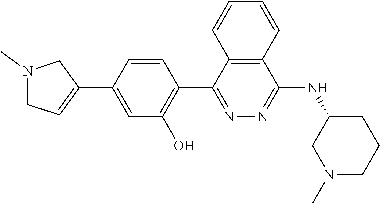 [SMILES: CN1CC=C(c2ccc(-c3nnc(N[C@@H]4CCCN(C)C4)c4ccccc34)c(O)c2)C1] | **** |
| I-59 | 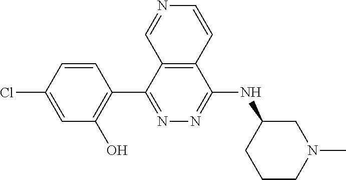 [SMILES: CN1CCC[C@@H](Nc2nnc(-c3ccc(Cl)cc3O)c3cnccc23)C1] | ***** |
| I-60 | 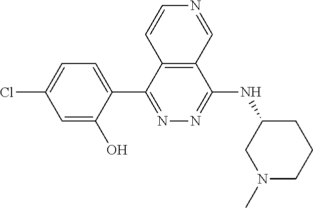 [SMILES: CN1CCC[C@@H](Nc2nnc(-c3ccc(Cl)cc3O)c3ccncc23)C1] | ***** |
| I-61 | 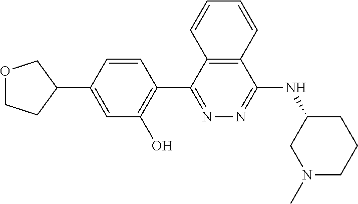 [SMILES: CN1CCC[C@@H](Nc2nnc(-c3ccc(C4CCOC4)cc3O)c3ccccc23)C1] | **** |
| I-62 | 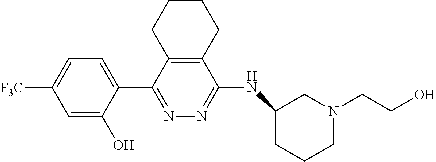 [SMILES: OCCN1CCC[C@@H](Nc2nnc(-c3ccc(C(F)(F)F)cc3O)c3c2CCCC3)C1] | ***** |
| I-63 | 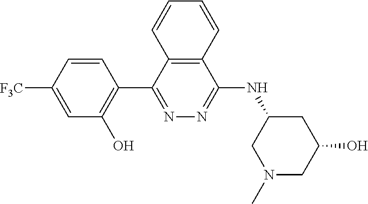 [SMILES: CN1C[C@@H](O)C[C@@H](Nc2nnc(-c3ccc(C(F)(F)F)cc3O)c3ccccc23)C1] | **** |
| I-64 | 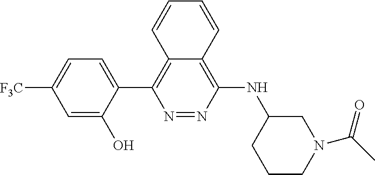 [SMILES: CC(=O)N1CCCC(Nc2nnc(-c3ccc(C(F)(F)F)cc3O)c3ccccc23)C1] | *** |
| I-65 | 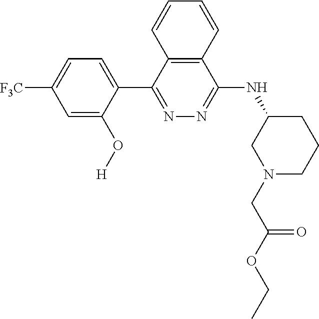 [SMILES: CCOC(=O)CN1CCC[C@@H](Nc2nnc(-c3ccc(C(F)(F)F)cc3O)c3ccccc23)C1] | ***** |
| I-66 | 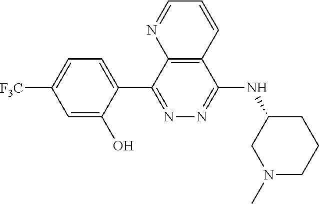 [SMILES: CN1CCC[C@@H](Nc2nnc(-c3ccc(C(F)(F)F)cc3O)c3ncccc23)C1] | **** |
| I-67 | 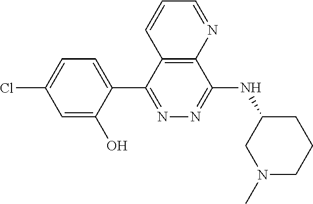 [SMILES: CN1CCC[C@@H](Nc2nnc(-c3ccc(Cl)cc3O)c3cccnc23)C1] | ***** |
| I-68 | 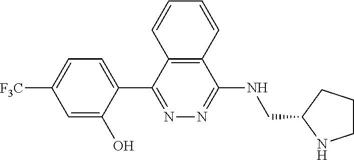 [SMILES: Oc1cc(C(F)(F)F)ccc1-c1nnc(NC[C@@H]2CCCN2)c2ccccc12] | ***** |
| I-70 | 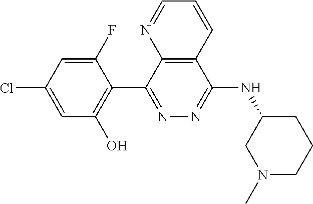 [SMILES: CN1CCC[C@@H](Nc2nnc(-c3c(O)cc(Cl)cc3F)c3ncccc23)C1] | ***** |
| I-71 | 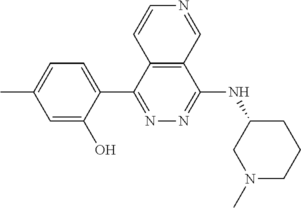 [SMILES: Cc1ccc(-c2nnc(N[C@@H]3CCCN(C)C3)c3cnccc23)c(O)c1] | ***** |
| I-72 | 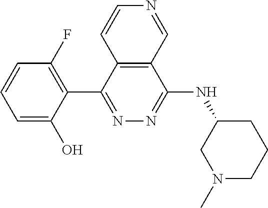 [SMILES: CN1CCC[C@@H](Nc2nnc(-c3c(O)cccc3F)c3ccncc23)C1] | **** |
| I-73 | 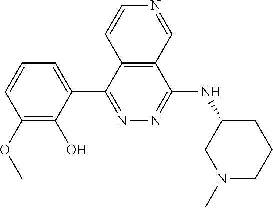 [SMILES: COc1cccc(-c2nnc(N[C@@H]3CCCN(C)C3)c3cnccc23)c1O] | ***** |
| I-74 | 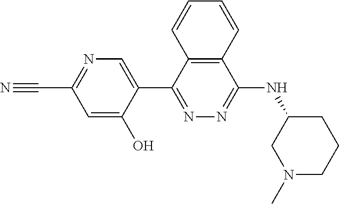 [SMILES: CN1CCC[C@@H](Nc2nnc(-c3cnc(C#N)cc3O)c3ccccc23)C1] | *** |
| I-75 | 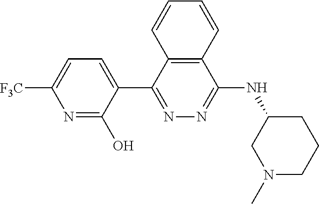 [SMILES: CN1CCC[C@@H](Nc2nnc(-c3ccc(C(F)(F)F)nc3O)c3ccccc23)C1] | *** |
| I-76 | 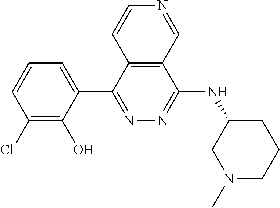 [SMILES: CN1CCC[C@@H](Nc2nnc(-c3cccc(Cl)c3O)c3ccncc23)C1] | ***** |
| I-77 | 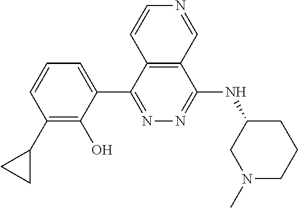 [SMILES: CN1CCC[C@@H](Nc2nnc(-c3cccc(C4CC4)c3O)c3ccncc23)C1] | ***** |
| I-78 | 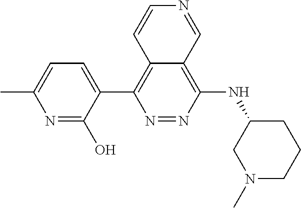 [SMILES: Cc1ccc(-c2nnc(N[C@@H]3CCCN(C)C3)c3cnccc23)c(O)n1] | *** |
| I-79 | 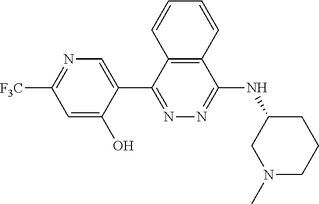 [SMILES: CN1CCC[C@@H](Nc2nnc(-c3cnc(C(F)(F)F)cc3O)c3ccccc23)C1] | *** |
| I-80 | 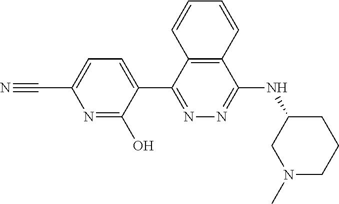 [SMILES: CN1CCC[C@@H](Nc2nnc(-c3ccc(C#N)nc3O)c3ccccc23)C1] | ** |
| I-81 | 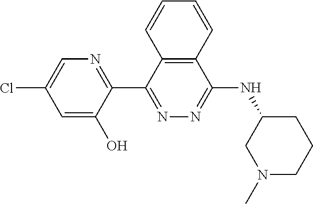 [SMILES: CN1CCC[C@@H](Nc2nnc(-c3ncc(Cl)cc3O)c3ccccc23)C1] | *** |
| I-82 | 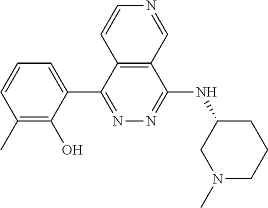 [SMILES: Cc1cccc(-c2nnc(N[C@@H]3CCCN(C)C3)c3cnccc23)c1O] | ***** |
| I-83 | 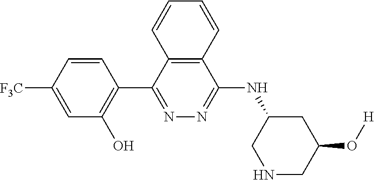 [SMILES: Oc1cc(C(F)(F)F)ccc1-c1nnc(N[C@H]2CNC[C@H](O)C2)c2ccccc12] | *** |
| I-84 | 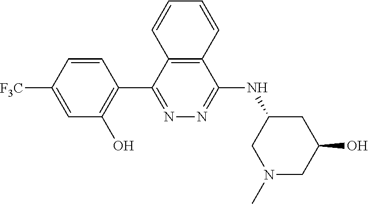 [SMILES: CN1C[C@H](O)C[C@@H](Nc2nnc(-c3ccc(C(F)(F)F)cc3O)c3ccccc23)C1] | *** |
| I-85 | 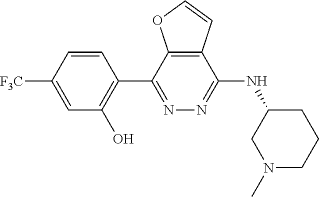 [SMILES: CN1CCC[C@@H](Nc2nnc(-c3ccc(C(F)(F)F)cc3O)c3occc23)C1] | *** |
| I-86 | 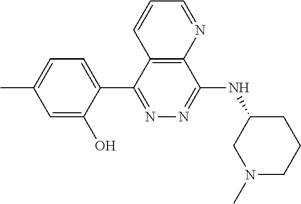 [SMILES: Cc1ccc(-c2nnc(N[C@@H]3CCCN(C)C3)c3ncccc23)c(O)c1] | ***** |
| I-87 | 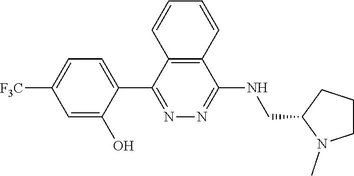 [SMILES: CN1CCC[C@H]1CNc1nnc(-c2ccc(C(F)(F)F)cc2O)c2ccccc12] | **** |
| I-88 | 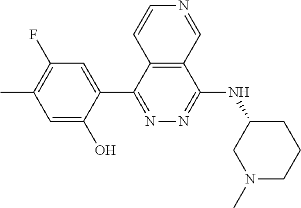 [SMILES: Cc1cc(O)c(-c2nnc(N[C@@H]3CCCN(C)C3)c3cnccc23)cc1F] | ***** |
| I-89 | 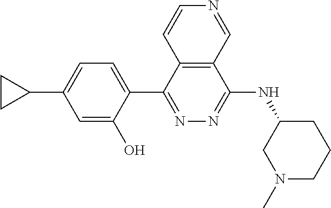 [SMILES: CN1CCC[C@@H](Nc2nnc(-c3ccc(C4CC4)cc3O)c3ccncc23)C1] | ***** |
| I-90 | 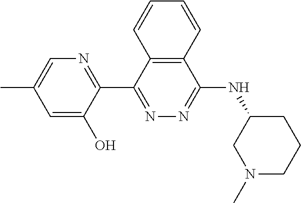 [SMILES: Cc1cnc(-c2nnc(N[C@@H]3CCCN(C)C3)c3ccccc23)c(O)c1] | **** |
| I-91 | 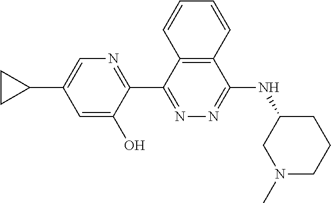 [SMILES: CN1CCC[C@@H](Nc2nnc(-c3ncc(C4CC4)cc3O)c3ccccc23)C1] | ***** |
| I-92 | 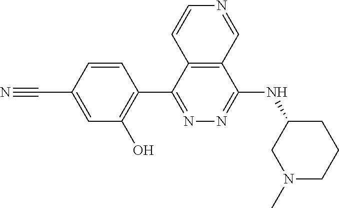 [SMILES: CN1CCC[C@@H](Nc2nnc(-c3ccc(C#N)cc3O)c3ccncc23)C1] | ***** |
| I-93 | 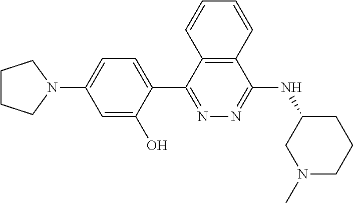 [SMILES: CN1CCC[C@@H](Nc2nnc(-c3ccc(N4CCCC4)cc3O)c3ccccc23)C1] | **** |
| I-94 | 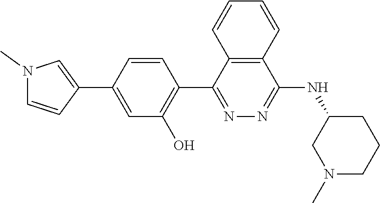 [SMILES: CN1CCC[C@@H](Nc2nnc(-c3ccc(-c4ccn(C)c4)cc3O)c3ccccc23)C1] | **** |
| I-95 | 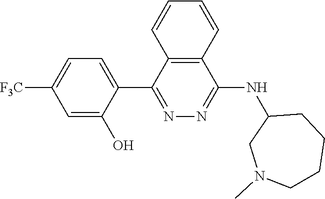 [SMILES: CN1CCCCC(Nc2nnc(-c3ccc(C(F)(F)F)cc3O)c3ccccc23)C1] | **** |
| I-96 | 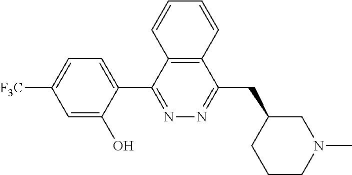 [SMILES: CN1CCC[C@@H](Cc2nnc(-c3ccc(C(F)(F)F)cc3O)c3ccccc23)C1] | * |
| I-97 | 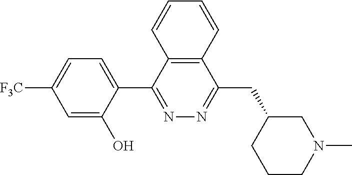 [SMILES: CN1CCC[C@H](Cc2nnc(-c3ccc(C(F)(F)F)cc3O)c3ccccc23)C1] | ***** |
| I-98 | 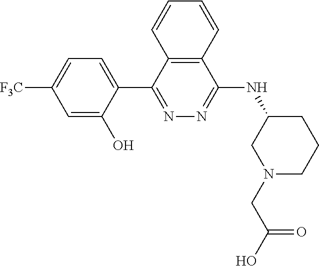 [SMILES: O=C(O)CN1CCC[C@@H](Nc2nnc(-c3ccc(C(F)(F)F)cc3O)c3ccccc23)C1] | ***** |
| I-99 | 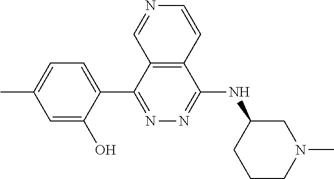 | ***** |
| I-100 | 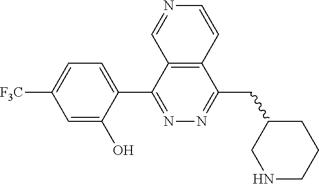 [SMILES: Oc1cc(C(F)(F)F)ccc1-c1nnc(CC2CCCNC2)c2ccncc12] | **** |
| I-101 | 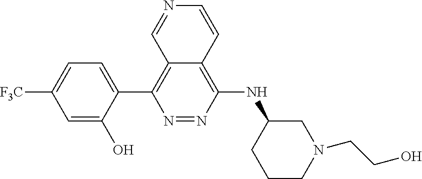 [SMILES: OCCN1CCC[C@@H](Nc2nnc(-c3ccc(C(F)(F)F)cc3O)c3cnccc23)C1] | ***** |
| I-102 | 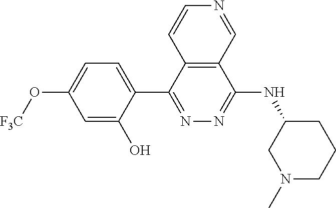 [SMILES: CN1CCC[C@@H](Nc2nnc(-c3ccc(OC(F)(F)F)cc3O)c3ccncc23)C1] | ***** |
| I-103 | 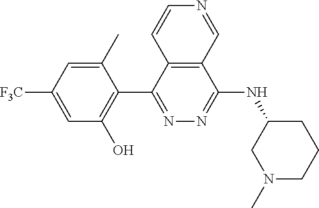 [SMILES: Cc1cc(C(F)(F)F)cc(O)c1-c1nnc(N[C@@H]2CCCN(C)C2)c2cnccc12] | ***** |
| I-104 | 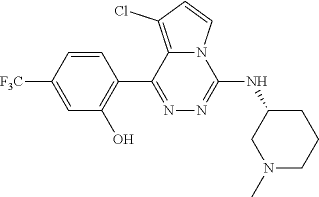 [SMILES: CN1CCC[C@@H](Nc2nnc(-c3ccc(C(F)(F)F)cc3O)c3c(Cl)ccn23)C1] | **** |
| I-105 | 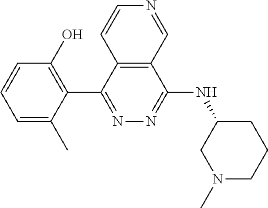 [SMILES: Cc1cccc(O)c1-c1nnc(N[C@@H]2CCCN(C)C2)c2cnccc12] | **** |
| I-106 | 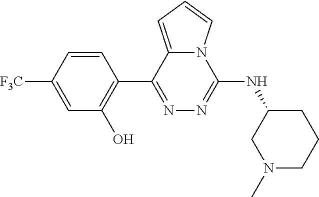 [SMILES: CN1CCC[C@@H](Nc2nnc(-c3ccc(C(F)(F)F)cc3O)c3cccn23)C1] | ***** |
| I-107 | 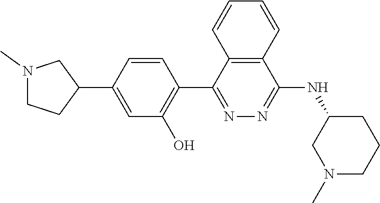 [SMILES: CN1CCC(c2ccc(-c3nnc(N[C@@H]4CCCN(C)C4)c4ccccc34)c(O)c2)C1] | *** |
| I-108 | 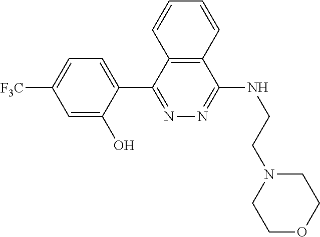 [SMILES: Oc1cc(C(F)(F)F)ccc1-c1nnc(NCCN2CCOCC2)c2ccccc12] | *** |
| I-109 | 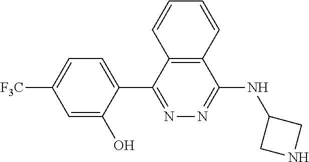 [SMILES: Oc1cc(C(F)(F)F)ccc1-c1nnc(NC2CNC2)c2ccccc12] | *** |
| I-110 | 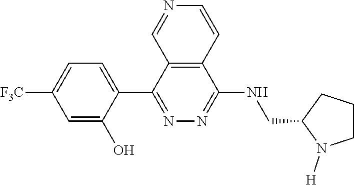 [SMILES: Oc1cc(C(F)(F)F)ccc1-c1nnc(NC[C@@H]2CCCN2)c2ccncc12] | ***** |
| I-111 | 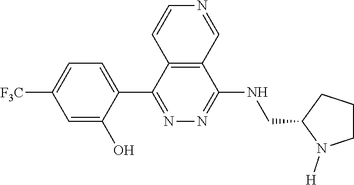 [SMILES: Oc1cc(C(F)(F)F)ccc1-c1nnc(NC[C@@H]2CCCN2)c2cnccc12] | **** |
| I-112 | 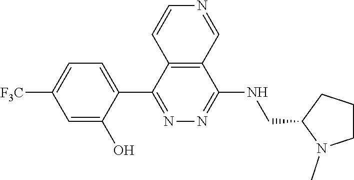 [SMILES: CN1CCC[C@H]1CNc1nnc(-c2ccc(C(F)(F)F)cc2O)c2ccncc12] | ***** |
| I-113 | 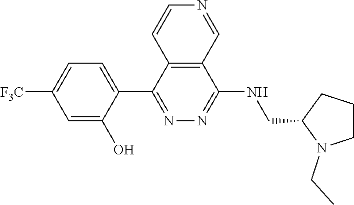 [SMILES: CCN1CCC[C@H]1CNc1nnc(-c2ccc(C(F)(F)F)cc2O)c2ccncc12] | ***** |
| I-115 | 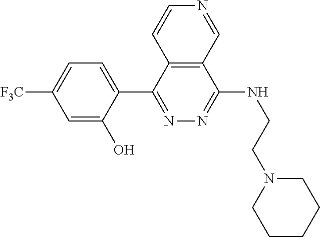 [SMILES: Oc1cc(C(F)(F)F)ccc1-c1nnc(NCCN2CCCCC2)c2cnccc12] | ** |
| I-116 | 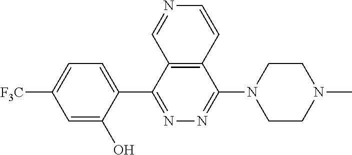 [SMILES: CN1CCN(c2nnc(-c3ccc(C(F)(F)F)cc3O)c3cnccc23)CC1] | ** |
| I-117 | 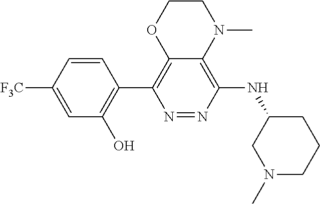 [SMILES: CN1CCC[C@@H](Nc2nnc(-c3ccc(C(F)(F)F)cc3O)c3c2N(C)CCO3)C1] | *** |
| I-118 | 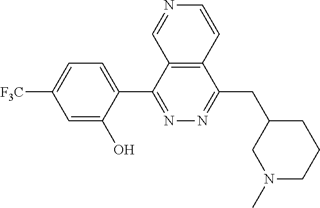 [SMILES: CN1CCCC(Cc2nnc(-c3ccc(C(F)(F)F)cc3O)c3cnccc23)C1] | **** |
| I-119 | 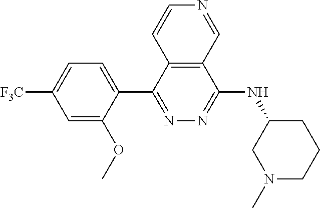 [SMILES: COc1cc(C(F)(F)F)ccc1-c1nnc(N[C@@H]2CCCN(C)C2)c2cnccc12] | **** |
| I-120 | 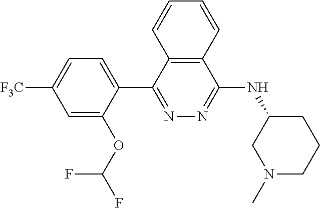 [SMILES: CN1CCC[C@@H](Nc2nnc(-c3ccc(C(F)(F)F)cc3OC(F)F)c3ccccc23)C1] | **** |
| I-121 | 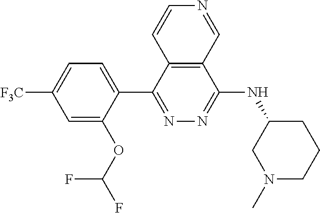 [SMILES: CN1CCC[C@@H](Nc2nnc(-c3ccc(C(F)(F)F)cc3OC(F)F)c3ccncc23)C1] | ***** |
| I-122 | 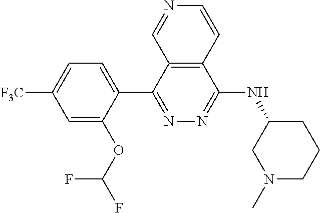 [SMILES: CN1CCC[C@@H](Nc2nnc(-c3ccc(C(F)(F)F)cc3OC(F)F)c3cnccc23)C1] | **** |
| I-123 | 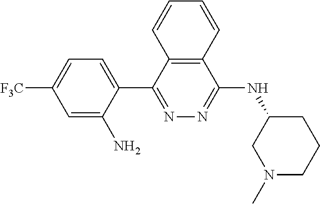 [SMILES: CN1CCC[C@@H](Nc2nnc(-c3ccc(C(F)(F)F)cc3N)c3ccccc23)C1] | **** |
| I-124 | 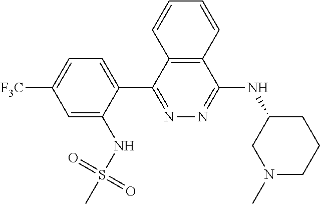 [SMILES: CN1CCC[C@@H](Nc2nnc(-c3ccc(C(F)(F)F)cc3NS(C)(=O)=O)c3ccccc23)C1] | **** |
| I-125 | 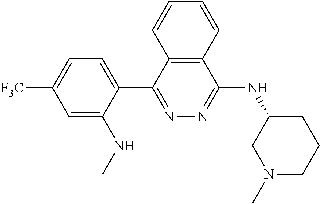 [SMILES: CNc1cc(C(F)(F)F)ccc1-c1nnc(N[C@@H]2CCCN(C)C2)c2ccccc12] | **** |
| I-126 | 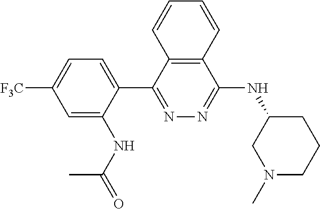 [SMILES: CC(=O)Nc1cc(C(F)(F)F)ccc1-c1nnc(N[C@@H]2CCCN(C)C2)c2ccccc12] | **** |
| I-127 | 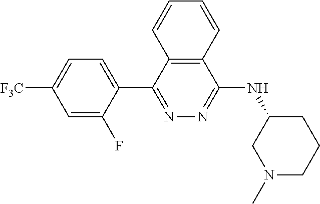 [SMILES: CN1CCC[C@@H](Nc2nnc(-c3ccc(C(F)(F)F)cc3F)c3ccccc23)C1] | **** |
| I-128 | 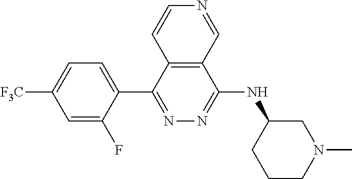 [SMILES: CN1CCC[C@@H](Nc2nnc(-c3ccc(C(F)(F)F)cc3F)c3ccncc23)C1] | **** |
| I-129 | 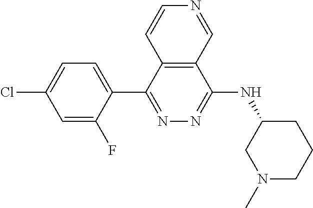 [SMILES: CN1CCC[C@@H](Nc2nnc(-c3ccc(Cl)cc3F)c3ccncc23)C1] | ***** |
| I-130 | 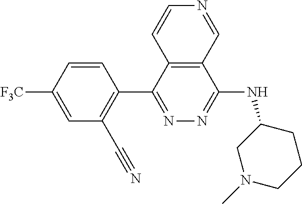 [SMILES: CN1CCC[C@@H](Nc2nnc(-c3ccc(C(F)(F)F)cc3C#N)c3ccncc23)C1] | **** |
| I-131 | 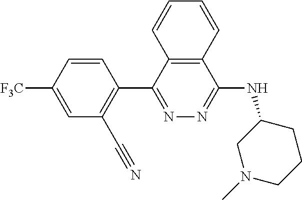 [SMILES: CN1CCC[C@@H](Nc2nnc(-c3ccc(C(F)(F)F)cc3C#N)c3ccccc23)C1] | **** |
| I-132 | 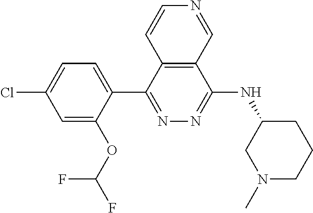 [SMILES: CN1CCC[C@@H](Nc2nnc(-c3ccc(Cl)cc3OC(F)F)c3ccncc23)C1] | ***** |
| I-133 | 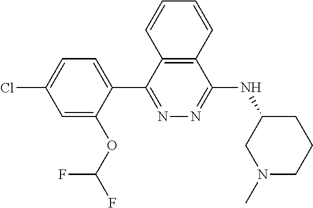 [SMILES: CN1CCC[C@@H](Nc2nnc(-c3ccc(Cl)cc3OC(F)F)c3ccccc23)C1] | **** |
| I-134 | 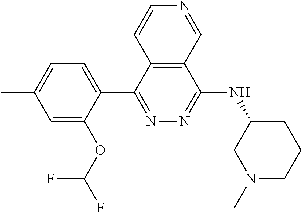 [SMILES: Cc1ccc(-c2nnc(N[C@@H]3CCCN(C)C3)c3cnccc23)c(OC(F)F)c1] | **** |
| I-135 | 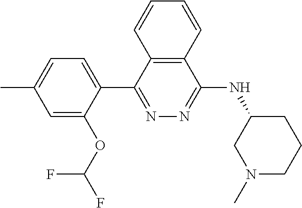 [SMILES: Cc1ccc(-c2nnc(N[C@@H]3CCCN(C)C3)c3ccccc23)c(OC(F)F)c1] | **** |
| I-136 | 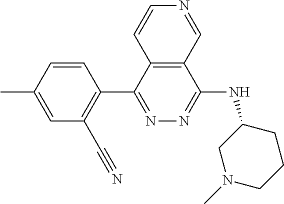 [SMILES: Cc1ccc(-c2nnc(N[C@@H]3CCCN(C)C3)c3cnccc23)c(C#N)c1] | **** |
| I-137 | 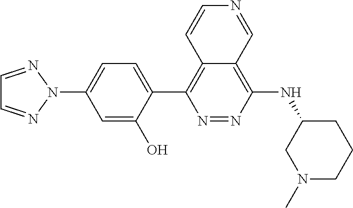 [SMILES: CN1CCC[C@@H](Nc2nnc(-c3ccc(-n4nccn4)cc3O)c3ccncc23)C1] | ***** |
| I-138 | 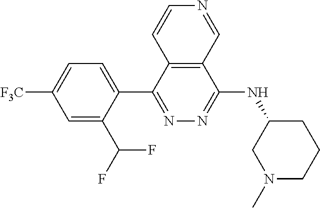 [SMILES: CN1CCC[C@@H](Nc2nnc(-c3ccc(C(F)(F)F)cc3C(F)F)c3ccncc23)C1] | **** |
| I-139 | 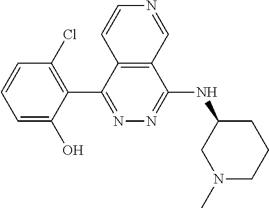 [SMILES: CN1CCC[C@H](Nc2nnc(-c3c(O)cccc3Cl)c3ccncc23)C1] | **** |
| I-140 | 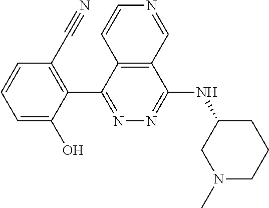 [SMILES: CN1CCC[C@@H](Nc2nnc(-c3c(O)cccc3C#N)c3ccncc23)C1] | **** |
| I-141 | 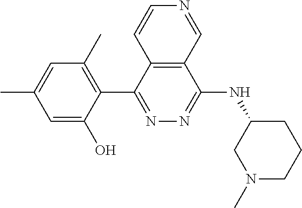 [SMILES: Cc1cc(C)c(-c2nnc(N[C@@H]3CCCN(C)C3)c3cnccc23)c(O)c1] | **** |
| I-142 | 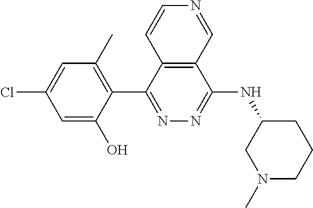 [SMILES: Cc1cc(Cl)cc(O)c1-c1nnc(N[C@@H]2CCCN(C)C2)c2cnccc12] | ***** |
| I-143 | 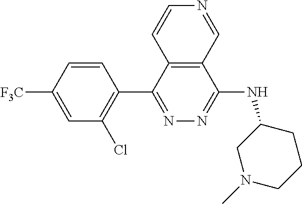 [SMILES: CN1CCC[C@@H](Nc2nnc(-c3ccc(C(F)(F)F)cc3Cl)c3ccncc23)C1] | **** |
| I-144 | 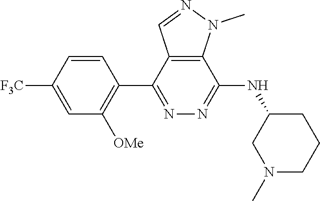 [SMILES: COc1cc(C(F)(F)F)ccc1-c1nnc(N[C@@H]2CCCN(C)C2)c2c1cnn2C] | **** |
| I-145 | 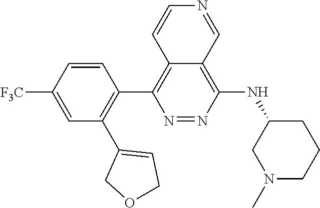 [SMILES: CN1CCC[C@@H](Nc2nnc(-c3ccc(C(F)(F)F)cc3C3=CCOC3)c3ccncc23)C1] | **** |
| I-146 | 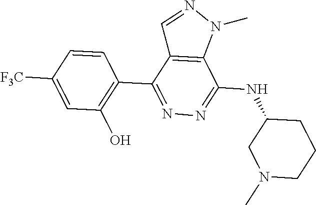 [SMILES: CN1CCC[C@@H](Nc2nnc(-c3ccc(C(F)(F)F)cc3O)c3cnn(C)c23)C1] | ***** |
| I-147 | 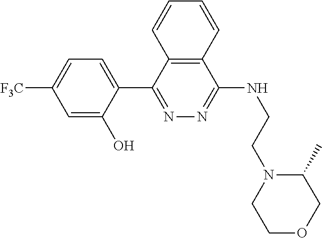 [SMILES: C[C@@H]1COCCN1CCNc1nnc(-c2ccc(C(F)(F)F)cc2O)c2ccccc12] | *** |
| I-148 | 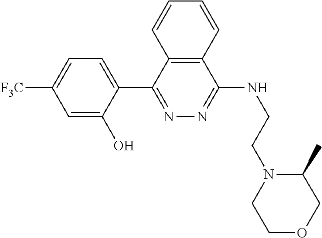 [SMILES: C[C@H]1COCCN1CCNc1nnc(-c2ccc(C(F)(F)F)cc2O)c2ccccc12] | *** |
| I-149 | 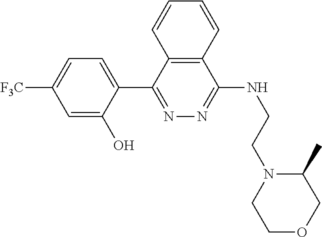 [SMILES: C[C@H]1COCCN1CCNc1nnc(-c2ccc(C(F)(F)F)cc2O)c2ccccc12] | *** |
| I-150 | 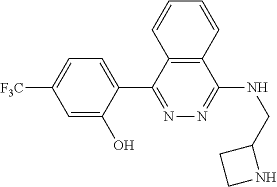 [SMILES: Oc1cc(C(F)(F)F)ccc1-c1nnc(NCC2CCN2)c2ccccc12] | *** |
| I-151 | 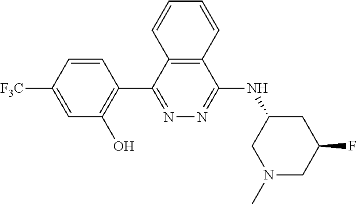 [SMILES: CN1C[C@H](F)C[C@@H](Nc2nnc(-c3ccc(C(F)(F)F)cc3O)c3ccccc23)C1] | ***** |
| I-152 | 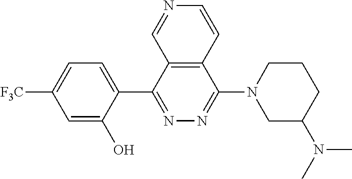 [SMILES: CN(C)C1CCCN(c2nnc(-c3ccc(C(F)(F)F)cc3O)c3cnccc23)C1] | **** |
| I-153 | 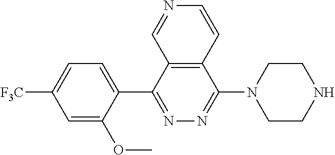 [SMILES: COc1cc(C(F)(F)F)ccc1-c1nnc(N2CCNCC2)c2ccncc12] | *** |
| I-154 | 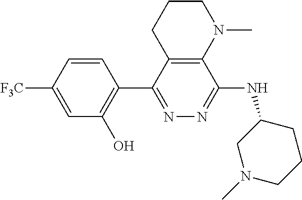 [SMILES: CN1CCC[C@@H](Nc2nnc(-c3ccc(C(F)(F)F)cc3O)c3c2N(C)CCC3)C1] | **** |
| I-155 | 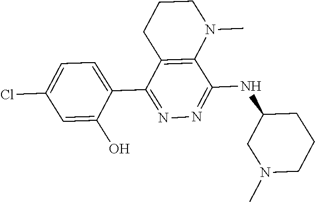 [SMILES: CN1CCC[C@H](Nc2nnc(-c3ccc(Cl)cc3O)c3c2N(C)CCC3)C1] | **** |
| I-156 | 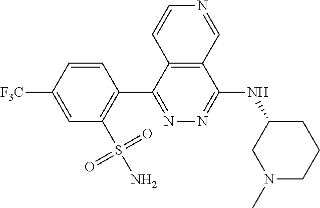 [SMILES: CN1CCC[C@@H](Nc2nnc(-c3ccc(C(F)(F)F)cc3S(N)(=O)=O)c3ccncc23)C1] | ** |
| I-157 | 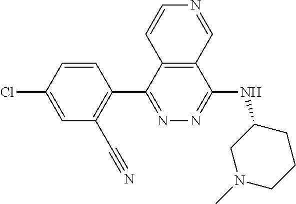 [SMILES: CN1CCC[C@@H](Nc2nnc(-c3ccc(Cl)cc3C#N)c3ccncc23)C1] | **** |
| I-158 | 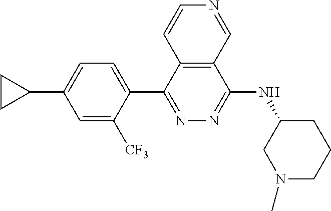 [SMILES: CN1CCC[C@@H](Nc2nnc(-c3ccc(C4CC4)cc3C(F)(F)F)c3ccncc23)C1] | ***** |
| I-159 | 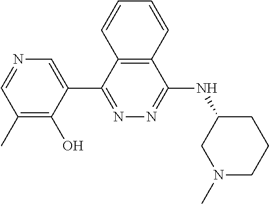 [SMILES: Cc1cncc(-c2nnc(N[C@@H]3CCCN(C)C3)c3ccccc23)c1O] | ** |
| I-160 | 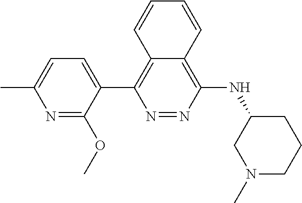 [SMILES: COc1nc(C)ccc1-c1nnc(N[C@@H]2CCCN(C)C2)c2ccccc12] | ** |
| I-161 | 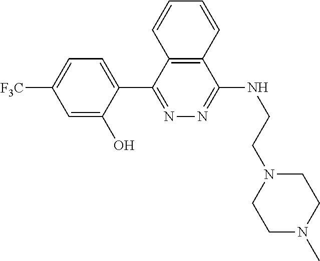 [SMILES: CN1CCN(CCNc2nnc(-c3ccc(C(F)(F)F)cc3O)c3ccccc23)CC1] | *** |
| I-162 | 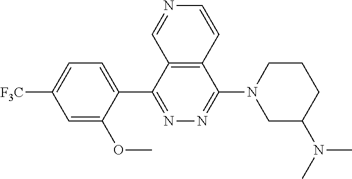 [SMILES: COc1cc(C(F)(F)F)ccc1-c1nnc(N2CCCC(N(C)C)C2)c2ccncc12] | *** |
| I-163 | 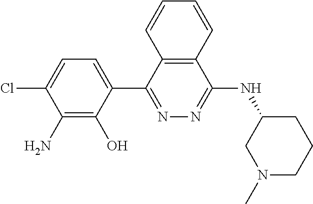 [SMILES: CN1CCC[C@@H](Nc2nnc(-c3ccc(Cl)c(N)c3O)c3ccccc23)C1] | *** |
| I-164 | 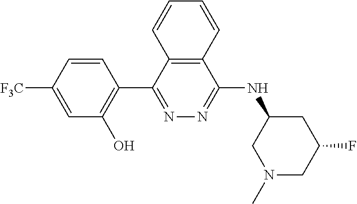 [SMILES: CN1C[C@@H](F)C[C@H](Nc2nnc(-c3ccc(C(F)(F)F)cc3O)c3ccccc23)C1] | *** |
| I-165 | 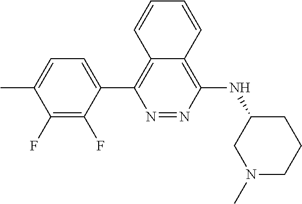 [SMILES: Cc1ccc(-c2nnc(N[C@@H]3CCCN(C)C3)c3ccccc23)c(F)c1F] | *** |
| I-166 | 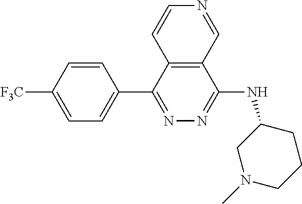 [SMILES: CN1CCC[C@@H](Nc2nnc(-c3ccc(C(F)(F)F)cc3)c3ccncc23)C1] | **** |
| I-167 | 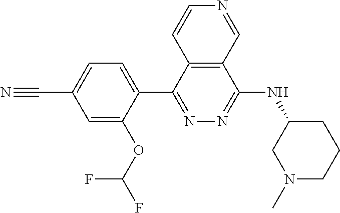 [SMILES: CN1CCC[C@@H](Nc2nnc(-c3ccc(C#N)cc3OC(F)F)c3ccncc23)C1] | *** |
| I-168 | 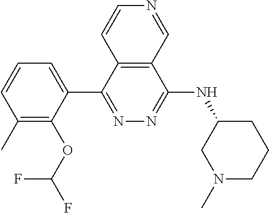 [SMILES: Cc1cccc(-c2nnc(N[C@@H]3CCCN(C)C3)c3cnccc23)c1OC(F)F] | *** |
| I-169 | 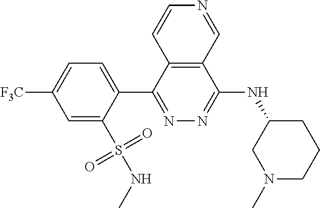 [SMILES: CNS(=O)(=O)c1cc(C(F)(F)F)ccc1-c1nnc(N[C@@H]2CCCN(C)C2)c2cnccc12] | *** |
| I-170 | 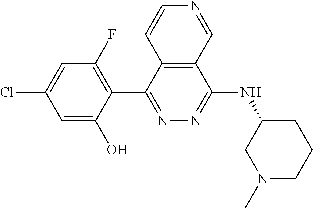 [SMILES: CN1CCC[C@@H](Nc2nnc(-c3c(O)cc(Cl)cc3F)c3ccncc23)C1] | ***** |
| I-171 | 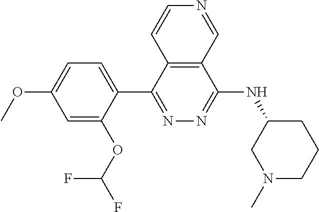 [SMILES: COc1ccc(-c2nnc(N[C@@H]3CCCN(C)C3)c3cnccc23)c(OC(F)F)c1] | ***** |
| I-172 | 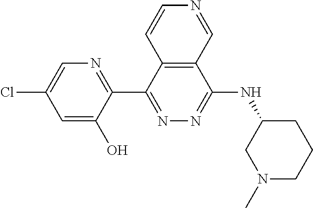 [SMILES: CN1CCC[C@@H](Nc2nnc(-c3ncc(Cl)cc3O)c3ccncc23)C1] | **** |
| I-173 | 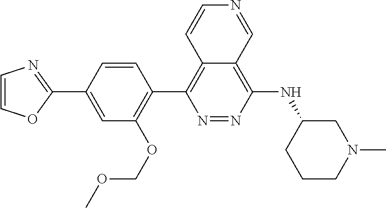 [SMILES: COCOc1cc(-c2ncco2)ccc1-c1nnc(N[C@H]2CCCN(C)C2)c2cnccc12] | *** |
| I-174 | 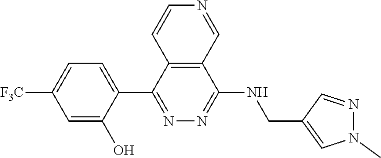 [SMILES: Cn1cc(CNc2nnc(-c3ccc(C(F)(F)F)cc3O)c3ccncc23)cn1] | *** |
| I-175 | 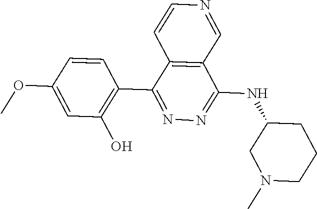 [SMILES: COc1ccc(-c2nnc(N[C@@H]3CCCN(C)C3)c3cnccc23)c(O)c1] | ***** |
| I-176 | 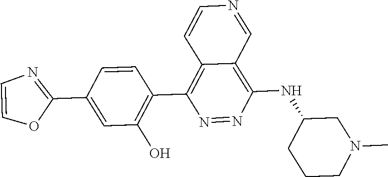 [SMILES: CN1CCC[C@H](Nc2nnc(-c3ccc(-c4ncco4)cc3O)c3ccncc23)C1] | **** |
| I-177 | 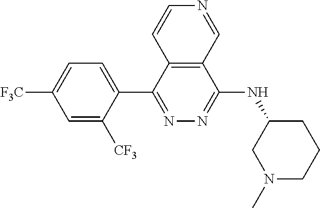 [SMILES: CN1CCC[C@@H](Nc2nnc(-c3ccc(C(F)(F)F)cc3C(F)(F)F)c3ccncc23)C1] | **** |
| I-178 | 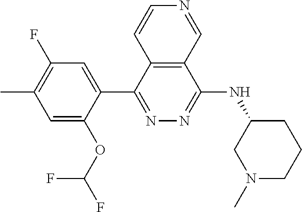 [SMILES: Cc1cc(OC(F)F)c(-c2nnc(N[C@@H]3CCCN(C)C3)c3cnccc23)cc1F] | **** |
| I-179 | 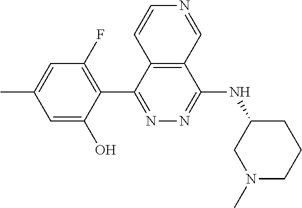 [SMILES: Cc1cc(O)c(-c2nnc(N[C@@H]3CCCN(C)C3)c3cnccc23)c(F)c1] | ***** |
| I-180 | 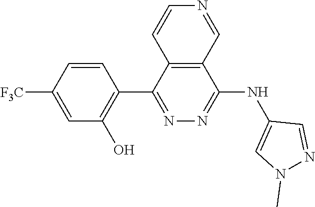 [SMILES: Cn1cc(Nc2nnc(-c3ccc(C(F)(F)F)cc3O)c3ccncc23)cn1] | **** |
| I-181 | 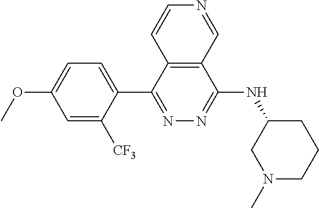 [SMILES: COc1ccc(-c2nnc(N[C@@H]3CCCN(C)C3)c3cnccc23)c(C(F)(F)F)c1] | **** |
| I-182 | 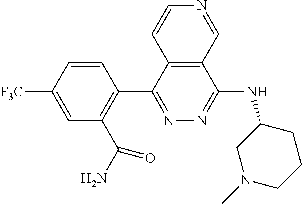 [SMILES: CN1CCC[C@@H](Nc2nnc(-c3ccc(C(F)(F)F)cc3C(N)=O)c3ccncc23)C1] | *** |
| I-183 | 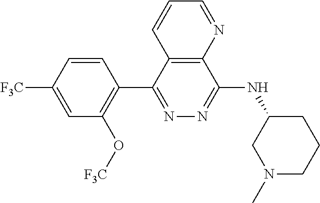 [SMILES: CN1CCC[C@@H](Nc2nnc(-c3ccc(C(F)(F)F)cc3OC(F)(F)F)c3cccnc23)C1] | **** |
| I-184 | 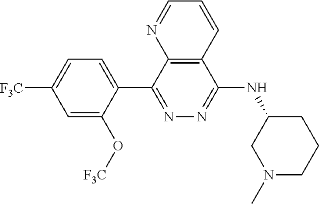 [SMILES: CN1CCC[C@@H](Nc2nnc(-c3ccc(C(F)(F)F)cc3OC(F)(F)F)c3ncccc23)C1] | *** |
| I-185 | 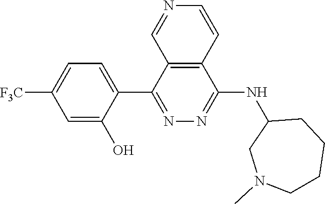 [SMILES: CN1CCCCC(Nc2nnc(-c3ccc(C(F)(F)F)cc3O)c3cnccc23)C1] | *** |
| I-186 | 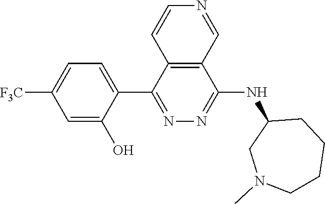 [SMILES: CN1CCCC[C@H](Nc2nnc(-c3ccc(C(F)(F)F)cc3O)c3ccncc23)C1] | *** |
| I-187 | 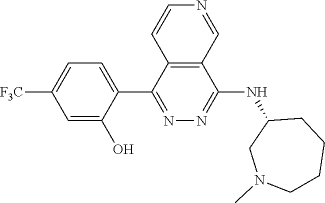 [SMILES: CN1CCCC[C@@H](Nc2nnc(-c3ccc(C(F)(F)F)cc3O)c3ccncc23)C1] | ***** |
| I-188 | 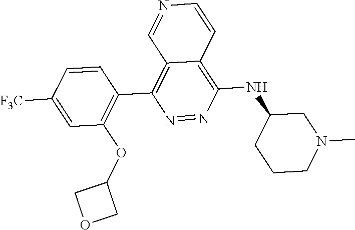 [SMILES: CN1CCC[C@@H](Nc2nnc(-c3ccc(C(F)(F)F)cc3OC3COC3)c3cnccc23)C1] | *** |
| I-189 | 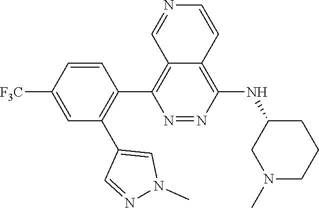 [SMILES: CN1CCC[C@@H](Nc2nnc(-c3ccc(C(F)(F)F)cc3-c3cnn(C)c3)c3cnccc23)C1] | *** |
| I-190 | 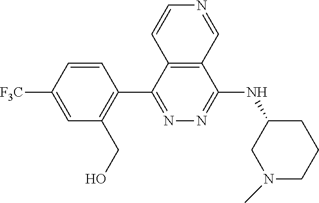 [SMILES: CN1CCC[C@@H](Nc2nnc(-c3ccc(C(F)(F)F)cc3CO)c3ccncc23)C1] | *** |
| I-191 | 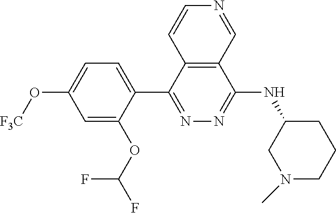 [SMILES: CN1CCC[C@@H](Nc2nnc(-c3ccc(OC(F)(F)F)cc3OC(F)F)c3ccncc23)C1] | **** |
| I-192 | 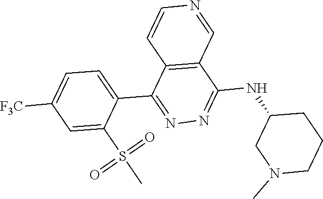 [SMILES: CN1CCC[C@@H](Nc2nnc(-c3ccc(C(F)(F)F)cc3S(C)(=O)=O)c3ccncc23)C1] | *** |
| I-193 | 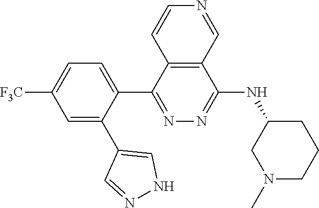 [SMILES: CN1CCC[C@@H](Nc2nnc(-c3ccc(C(F)(F)F)cc3-c3cn[nH]c3)c3ccncc23)C1] | *** |
| I-194 | 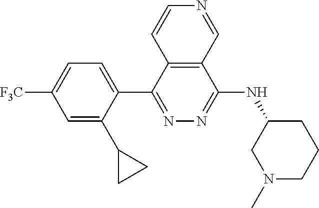 [SMILES: CN1CCC[C@@H](Nc2nnc(-c3ccc(C(F)(F)F)cc3C3CC3)c3ccncc23)C1] | **** |
| I-195 | 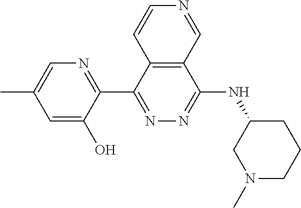 [SMILES: Cc1cnc(-c2nnc(N[C@@H]3CCCN(C)C3)c3cnccc23)c(O)c1] | **** |
| I-196 | 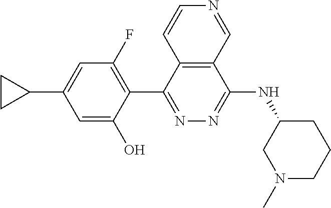 [SMILES: CN1CCC[C@@H](Nc2nnc(-c3c(O)cc(C4CC4)cc3F)c3ccncc23)C1] | ***** |
| I-197 | 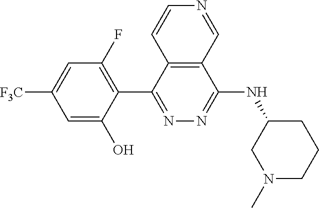 [SMILES: CN1CCC[C@@H](Nc2nnc(-c3c(O)cc(C(F)(F)F)cc3F)c3ccncc23)C1] | ***** |
| I-198 | 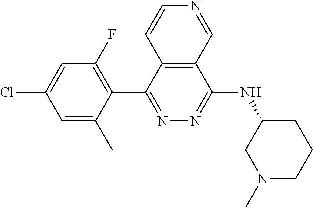 [SMILES: Cc1cc(Cl)cc(F)c1-c1nnc(N[C@@H]2CCCN(C)C2)c2cnccc12] | *** |
| I-199 | 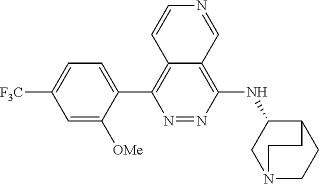 [SMILES: COc1cc(C(F)(F)F)ccc1-c1nnc(N[C@H]2CN3CCC2CC3)c2cnccc12] | *** |
| I-200 | 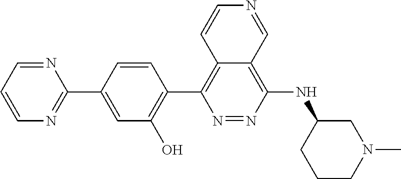 [SMILES: CN1CCC[C@@H](Nc2nnc(-c3ccc(-c4ncccn4)cc3O)c3ccncc23)C1] | **** |
| I-201 | 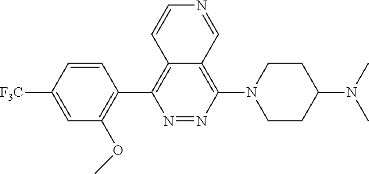 [SMILES: COc1cc(C(F)(F)F)ccc1-c1nnc(N2CCC(N(C)C)CC2)c2cnccc12] | *** |
| I-202 | 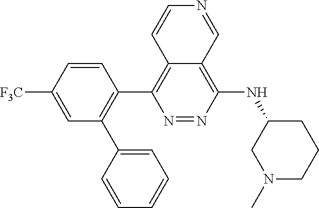 [SMILES: CN1CCC[C@@H](Nc2nnc(-c3ccc(C(F)(F)F)cc3-c3ccccc3)c3ccncc23)C1] | *** |
| I-203 | 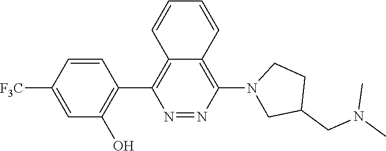 [SMILES: CN(C)CC1CCN(c2nnc(-c3ccc(C(F)(F)F)cc3O)c3ccccc23)C1] | *** |
| I-204 | 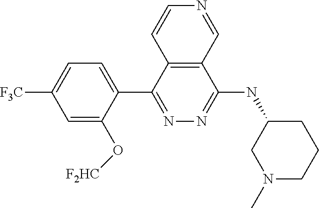 [SMILES: CN1CCC[C@@H](Nc2nnc(-c3ccc(C(F)(F)F)cc3OC(F)F)c3ccncc23)C1] | **** |
| I-205 | 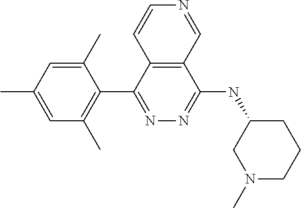 [SMILES: Cc1cc(C)c(-c2nnc(N[C@@H]3CCCN(C)C3)c3cnccc23)c(C)c1] | **** |
| I-206 | 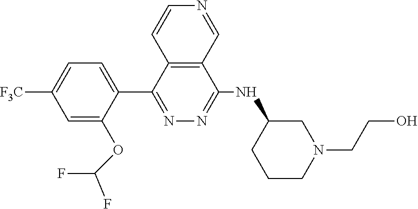 [SMILES: OCCN1CCC[C@@H](Nc2nnc(-c3ccc(C(F)(F)F)cc3OC(F)F)c3ccncc23)C1] | ***** |
| I-207 | 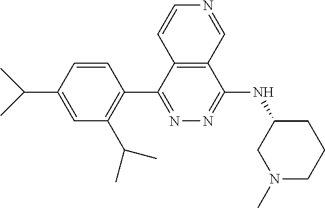 [SMILES: CC(C)c1ccc(-c2nnc(N[C@@H]3CCCN(C)C3)c3cnccc23)c(C(C)C)c1] | **** |
| I-208 | 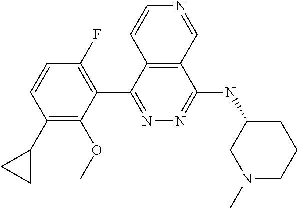 [SMILES: COc1c(C2CC2)ccc(F)c1-c1nnc(N[C@@H]2CCCN(C)C2)c2cnccc12] | *** |
| I-209 | 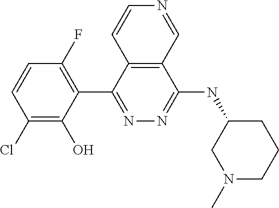 [SMILES: CN1CCC[C@@H](Nc2nnc(-c3c(F)ccc(Cl)c3O)c3ccncc23)C1] | **** |
| I-210 | 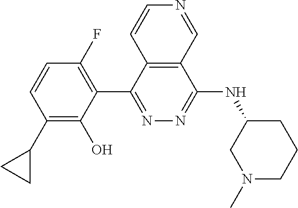 [SMILES: CN1CCC[C@@H](Nc2nnc(-c3c(F)ccc(C4CC4)c3O)c3ccncc23)C1] | *** |
| I-211 | 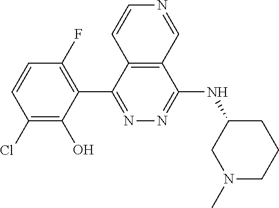 [SMILES: CN1CCC[C@@H](Nc2nnc(-c3c(F)ccc(Cl)c3O)c3ccncc23)C1] | *** |
| I-212 | 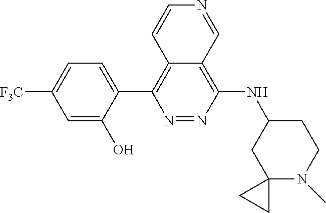 [SMILES: CN1CCC(Nc2nnc(-c3ccc(C(F)(F)F)cc3O)c3ccncc23)CC12CC2] | *** |
| I-213 | 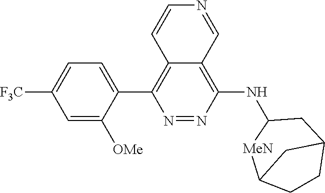 [SMILES: COc1cc(C(F)(F)F)ccc1-c1nnc(NC2CC3CCC(C3)N2C)c2cnccc12] | ** |
| I-214 | 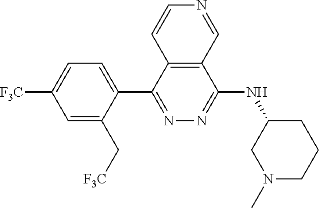 [SMILES: CN1CCC[C@@H](Nc2nnc(-c3ccc(C(F)(F)F)cc3CC(F)(F)F)c3ccncc23)C1] | ***** |
| I-215 | 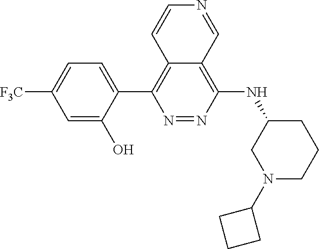 [SMILES: Oc1cc(C(F)(F)F)ccc1-c1nnc(N[C@@H]2CCCN(C3CCC3)C2)c2cnccc12] | ***** |
| I-216 | 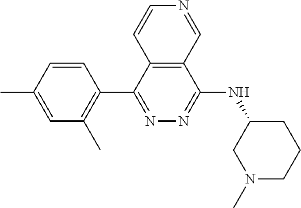 [SMILES: Cc1ccc(-c2nnc(N[C@@H]3CCCN(C)C3)c3cnccc23)c(C)c1] | **** |
| I-217 | 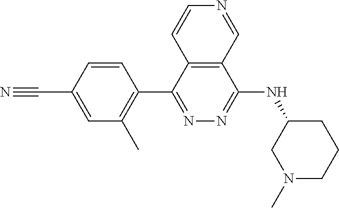 [SMILES: Cc1cc(C#N)ccc1-c1nnc(N[C@@H]2CCCN(C)C2)c2cnccc12] | *** |
| I-218 | 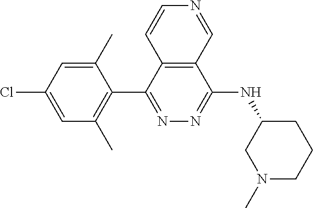 [SMILES: Cc1cc(Cl)cc(C)c1-c1nnc(N[C@@H]2CCCN(C)C2)c2cnccc12] | ***** |
| I-219 | 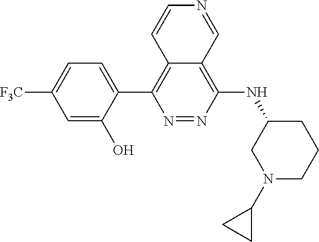 [SMILES: Oc1cc(C(F)(F)F)ccc1-c1nnc(N[C@@H]2CCCN(C3CC3)C2)c2cnccc12] | ***** |
| I-220 | 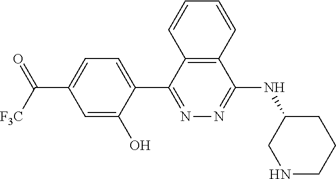 [SMILES: O=C(c1ccc(-c2nnc(N[C@@H]3CCCNC3)c3ccccc23)c(O)c1)C(F)(F)F] | ** |
| I-221 | 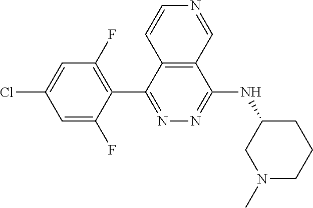 [SMILES: CN1CCC[C@@H](Nc2nnc(-c3c(F)cc(Cl)cc3F)c3ccncc23)C1] | **** |
| I-222 | 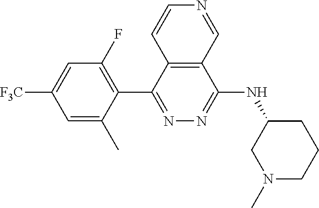 [SMILES: Cc1cc(C(F)(F)F)cc(F)c1-c1nnc(N[C@@H]2CCCN(C)C2)c2cnccc12] | **** |
| I-223 | 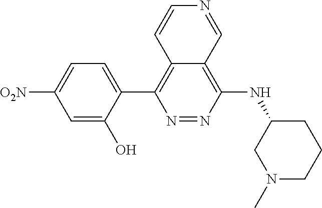 [SMILES: CN1CCC[C@@H](Nc2nnc(-c3ccc([N+](=O)[O-])cc3O)c3ccncc23)C1] | **** |
| I-224 | 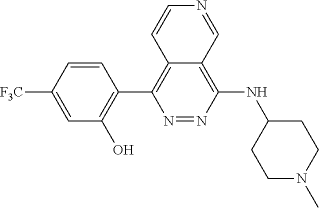 [SMILES: CN1CCC(Nc2nnc(-c3ccc(C(F)(F)F)cc3O)c3ccncc23)CC1] | *** |
| I-225 | 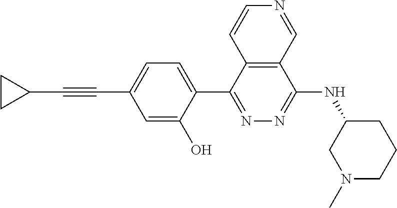 [SMILES: CN1CCC[C@@H](Nc2nnc(-c3ccc(C#CC4CC4)cc3O)c3ccncc23)C1] | **** |
| I-226 | 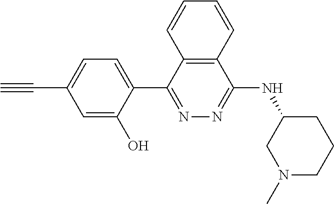 [SMILES: C#Cc1ccc(-c2nnc(N[C@@H]3CCCN(C)C3)c3ccccc23)c(O)c1] | ***** |
| I-227 | 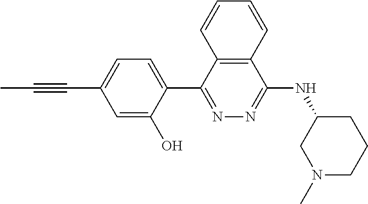 [SMILES: CC#Cc1ccc(-c2nnc(N[C@@H]3CCCN(C)C3)c3ccccc23)c(O)c1] | ***** |
| I-228 | 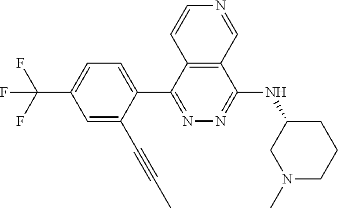 [SMILES: CC#Cc1cc(C(F)(F)F)ccc1-c1nnc(N[C@@H]2CCCN(C)C2)c2cnccc12] | **** |
| I-229 | 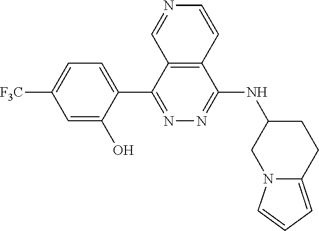 [SMILES: Oc1cc(C(F)(F)F)ccc1-c1nnc(NC2CCc3cccn3C2)c2ccncc12] | *** |
| I-230 | 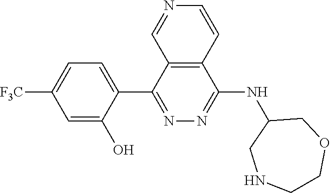 [SMILES: Oc1cc(C(F)(F)F)ccc1-c1nnc(NC2CNCCOC2)c2ccncc12] | *** |
| I-231 | 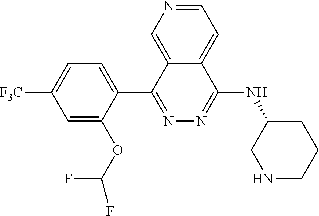 [SMILES: FC(F)Oc1cc(C(F)(F)F)ccc1-c1nnc(N[C@@H]2CCCNC2)c2ccncc12] | *** |
| I-232 | 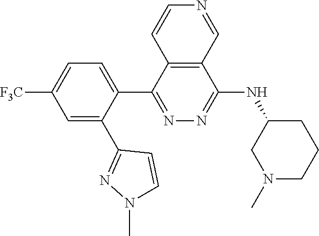 [SMILES: CN1CCC[C@@H](Nc2nnc(-c3ccc(C(F)(F)F)cc3-c3ccn(C)n3)c3ccncc23)C1] | *** |
| I-233 | 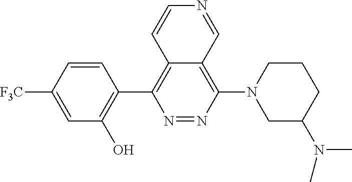 [SMILES: CN(C)C1CCCN(c2nnc(-c3ccc(C(F)(F)F)cc3O)c3ccncc23)C1] | *** |
| I-234 | 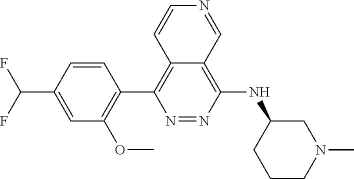 [SMILES: COc1cc(C(F)F)ccc1-c1nnc(N[C@@H]2CCCN(C)C2)c2cnccc12] | *** |
| I-235 | 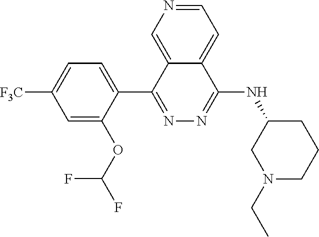 [SMILES: CCN1CCC[C@@H](Nc2nnc(-c3ccc(C(F)(F)F)cc3OC(F)F)c3cnccc23)C1] | **** |
| I-236 | 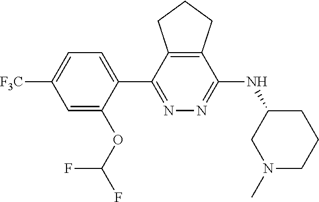 [SMILES: CN1CCC[C@@H](Nc2nnc(-c3ccc(C(F)(F)F)cc3OC(F)F)c3c2CCC3)C1] | **** |
| I-237 | 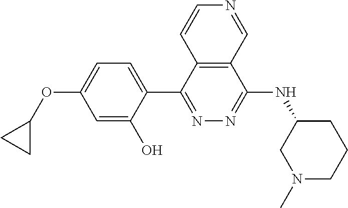 [SMILES: CN1CCC[C@@H](Nc2nnc(-c3ccc(OC4CC4)cc3O)c3ccncc23)C1] | **** |
| I-238 | 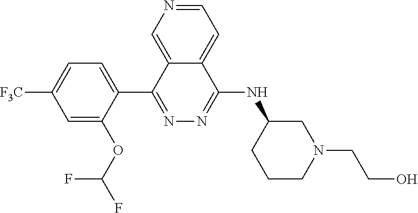 [SMILES: OCCN1CCC[C@@H](Nc2nnc(-c3ccc(C(F)(F)F)cc3OC(F)F)c3cnccc23)C1] | *** |
| I-239 | 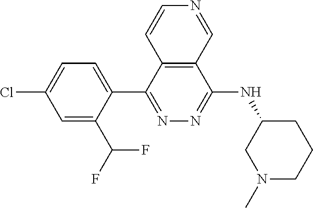 [SMILES: CN1CCC[C@@H](Nc2nnc(-c3ccc(Cl)cc3C(F)F)c3ccncc23)C1] | **** |
| I-240 | 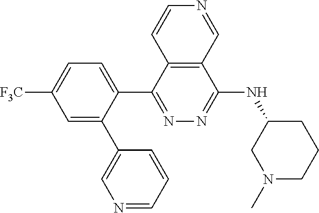 [SMILES: CN1CCC[C@@H](Nc2nnc(-c3ccc(C(F)(F)F)cc3-c3cccnc3)c3ccncc23)C1] | ** |
| I-241 | 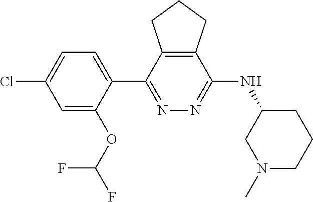 [SMILES: CN1CCC[C@@H](Nc2nnc(-c3ccc(Cl)cc3OC(F)F)c3c2CCC3)C1] | *** |
| I-242 | 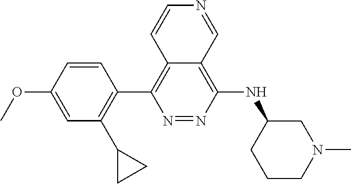 | **** |
| I-243 | 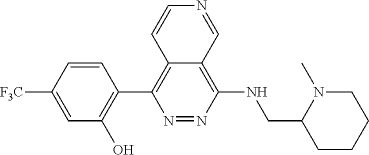 [SMILES: CN1CCCCC1CNc1nnc(-c2ccc(C(F)(F)F)cc2O)c2ccncc12] | **** |
| I-244 | 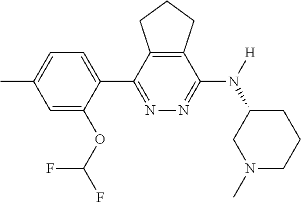 [SMILES: Cc1ccc(-c2nnc(N[C@@H]3CCCN(C)C3)c3c2CCC3)c(OC(F)F)c1] | *** |
| I-245 | 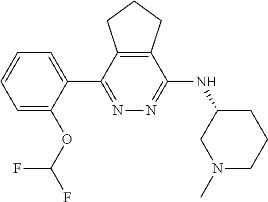 [SMILES: CN1CCC[C@@H](Nc2nnc(-c3ccccc3OC(F)F)c3c2CCC3)C1] | **** |
| I-246 | 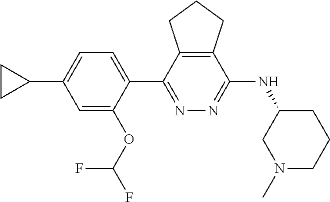 [SMILES: CN1CCC[C@@H](Nc2nnc(-c3ccc(C4CC4)cc3OC(F)F)c3c2CCC3)C1] | *** |
| I-247 | 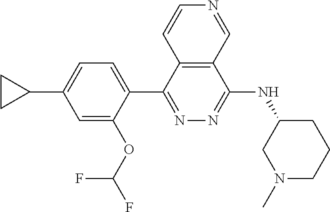 [SMILES: CN1CCC[C@@H](Nc2nnc(-c3ccc(C4CC4)cc3OC(F)F)c3ccncc23)C1] | **** |
| I-248 | 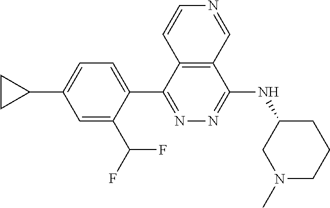 [SMILES: CN1CCC[C@@H](Nc2nnc(-c3ccc(C4CC4)cc3C(F)F)c3ccncc23)C1] | **** |
| I-249 | 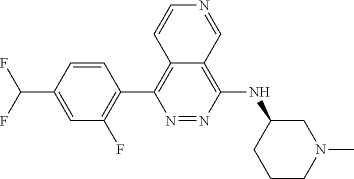 [SMILES: CN1CCC[C@@H](Nc2nnc(-c3ccc(C(F)F)cc3F)c3ccncc23)C1] | *** |
| I-250 | 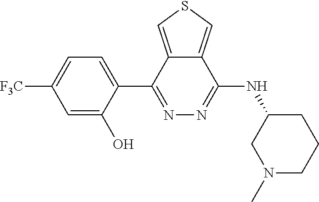 [SMILES: CN1CCC[C@@H](Nc2nnc(-c3ccc(C(F)(F)F)cc3O)c3cscc23)C1] | ***** |
| I-251 | 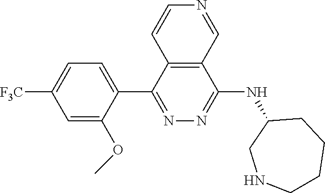 [SMILES: COc1cc(C(F)(F)F)ccc1-c1nnc(N[C@@H]2CCCCNC2)c2cnccc12] | ** |
| I-252 | 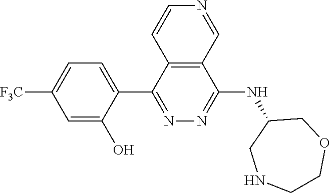 [SMILES: Oc1cc(C(F)(F)F)ccc1-c1nnc(N[C@H]2CNCCOC2)c2cnccc12] | *** |
| I-253 | 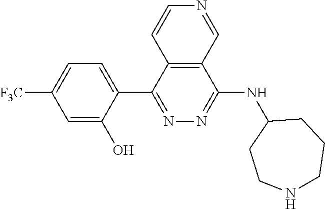 [SMILES: Oc1cc(C(F)(F)F)ccc1-c1nnc(NC2CCCNCC2)c2cnccc12] | **** |
| I-254 | 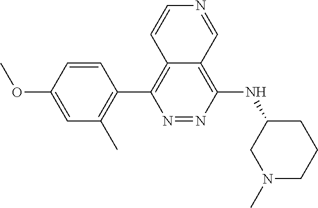 [SMILES: COc1ccc(-c2nnc(N[C@@H]3CCCN(C)C3)c3cnccc23)c(C)c1] | **** |
| I-255 | 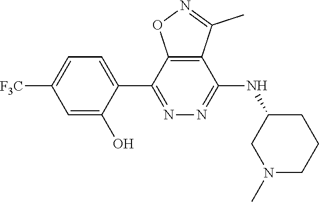 [SMILES: Cc1noc2c(-c3ccc(C(F)(F)F)cc3O)nnc(N[C@@H]3CCCN(C)C3)c12] | **** |
| I-256 | 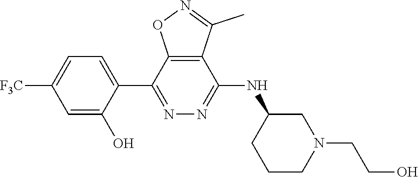 [SMILES: Cc1noc2c(-c3ccc(C(F)(F)F)cc3O)nnc(N[C@@H]3CCCN(CCO)C3)c12] | *** |
| I-257 | 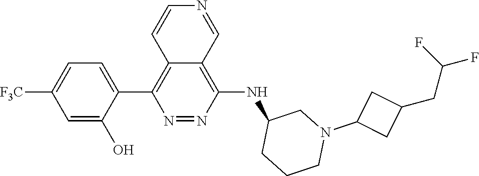 [SMILES: Oc1cc(C(F)(F)F)ccc1-c1nnc(N[C@@H]2CCCN(C3CC(CC(F)F)C3)C2)c2cnccc12] | **** |
| I-258 | 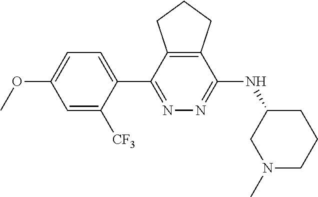 [SMILES: COc1ccc(-c2nnc(N[C@@H]3CCCN(C)C3)c3c2CCC3)c(C(F)(F)F)c1] | *** |
| I-259 | 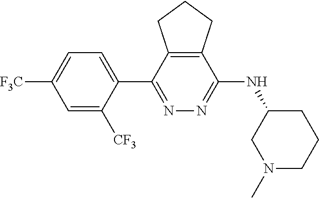 [SMILES: CN1CCC[C@@H](Nc2nnc(-c3ccc(C(F)(F)F)cc3C(F)(F)F)c3c2CCC3)C1] | *** |
| I-260 | 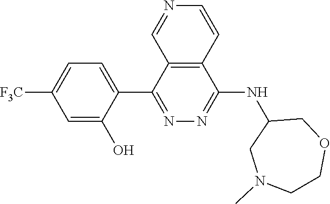 [SMILES: CN1CCOCC(Nc2nnc(-c3ccc(C(F)(F)F)cc3O)c3cnccc23)C1] | *** |
| I-261 | 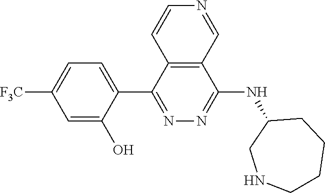 [SMILES: Oc1cc(C(F)(F)F)ccc1-c1nnc(N[C@@H]2CCCCNC2)c2cnccc12] | **** |
| I-262 | 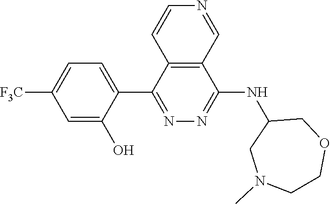 [SMILES: CN1CCOCC(Nc2nnc(-c3ccc(C(F)(F)F)cc3O)c3ccncc23)C1] | **** |
| I-263 | 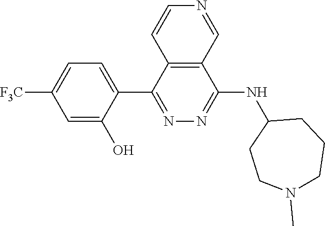 [SMILES: CN1CCCC(Nc2nnc(-c3ccc(C(F)(F)F)cc3O)c3ccncc23)CC1] | **** |
| I-264 | 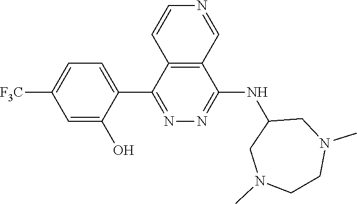 [SMILES: CN1CCN(C)CC(Nc2nnc(-c3ccc(C(F)(F)F)cc3O)c3ccncc23)C1] | *** |
| I-265 | 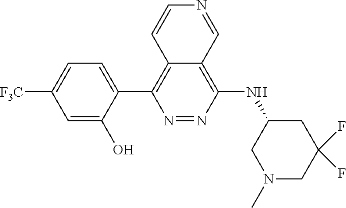 [SMILES: CN1C[C@H](Nc2nnc(-c3ccc(C(F)(F)F)cc3O)c3ccncc23)CC(F)(F)C1] | **** |
| I-266 | 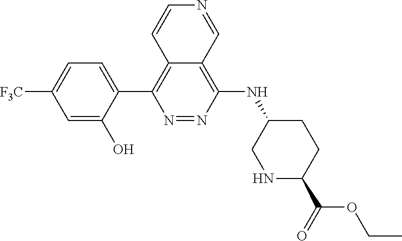 [SMILES: CCOC(=O)[C@@H]1CC[C@@H](Nc2nnc(-c3ccc(C(F)(F)F)cc3O)c3ccncc23)CN1] | ***** |
| I-267 | 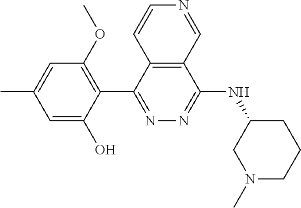 [SMILES: COc1cc(C)cc(O)c1-c1nnc(N[C@@H]2CCCN(C)C2)c2cnccc12] | **** |
| I-268 | 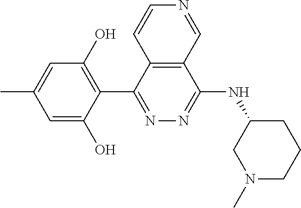 [SMILES: Cc1cc(O)c(-c2nnc(N[C@@H]3CCCN(C)C3)c3cnccc23)c(O)c1] | ***** |
| I-269 | 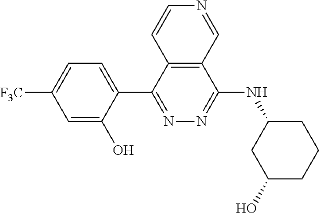 [SMILES: Oc1cc(C(F)(F)F)ccc1-c1nnc(N[C@@H]2CCC[C@H](O)C2)c2cnccc12] | **** |
| I-270 | 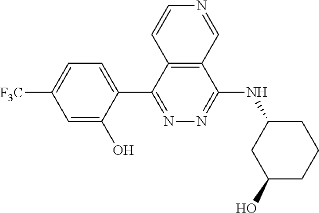 [SMILES: Oc1cc(C(F)(F)F)ccc1-c1nnc(N[C@@H]2CCC[C@@H](O)C2)c2cnccc12] | **** |
| I-271 | 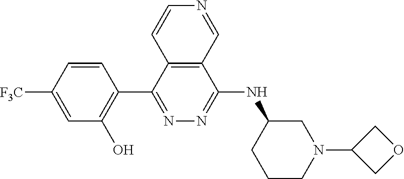 [SMILES: Oc1cc(C(F)(F)F)ccc1-c1nnc(N[C@@H]2CCCN(C3COC3)C2)c2cnccc12] | **** |
| I-272 | 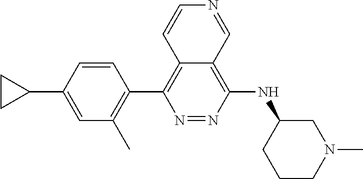 [SMILES: Cc1cc(C2CC2)ccc1-c1nnc(N[C@@H]2CCCN(C)C2)c2cnccc12] | **** |
| I-273 | 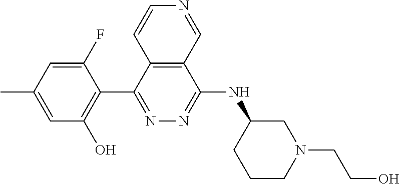 [SMILES: Cc1cc(O)c(-c2nnc(N[C@@H]3CCCN(CCO)C3)c3cnccc23)c(F)c1] | ***** |
| I-274 | 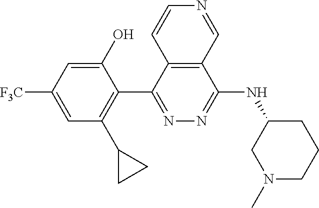 [SMILES: CN1CCC[C@@H](Nc2nnc(-c3c(O)cc(C(F)(F)F)cc3C3CC3)c3ccncc23)C1] | **** |
| I-275 | 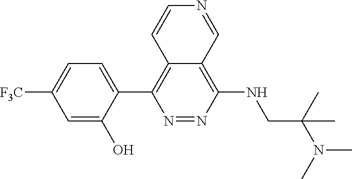 [SMILES: CN(C)C(C)(C)CNc1nnc(-c2ccc(C(F)(F)F)cc2O)c2ccncc12] | **** |
| I-276 | 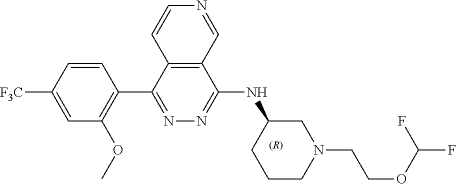 | *** |
| I-277 | 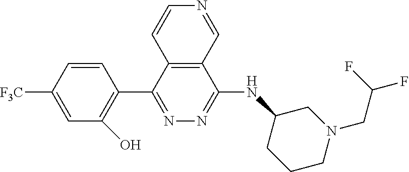 [SMILES: Oc1cc(C(F)(F)F)ccc1-c1nnc(N[C@@H]2CCCN(CC(F)F)C2)c2cnccc12] | ***** |
| I-278 | 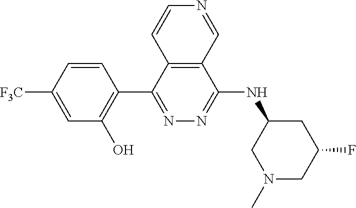 [SMILES: CN1C[C@@H](F)C[C@H](Nc2nnc(-c3ccc(C(F)(F)F)cc3O)c3ccncc23)C1] | *** |
| I-279 | 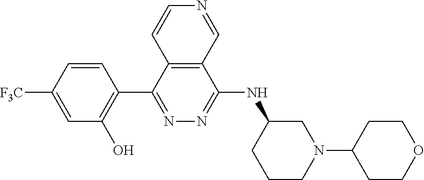 [SMILES: Oc1cc(C(F)(F)F)ccc1-c1nnc(N[C@@H]2CCCN(C3CCOCC3)C2)c2cnccc12] | ***** |
| I-280 | 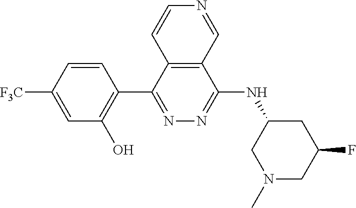 [SMILES: CN1C[C@H](F)C[C@@H](Nc2nnc(-c3ccc(C(F)(F)F)cc3O)c3ccncc23)C1] | ***** |
| I-281 | 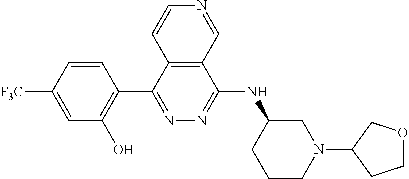 [SMILES: Oc1cc(C(F)(F)F)ccc1-c1nnc(N[C@@H]2CCCN(C3CCOC3)C2)c2cnccc12] | ***** |
| I-282 | 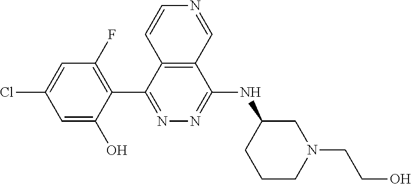 [SMILES: OCCN1CCC[C@@H](Nc2nnc(-c3c(O)cc(Cl)cc3F)c3ccncc23)C1] | ***** |
| I-283 | 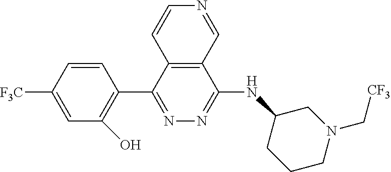 [SMILES: Oc1cc(C(F)(F)F)ccc1-c1nnc(N[C@@H]2CCCN(CC(F)(F)F)C2)c2cnccc12] | ***** |
| I-284 | 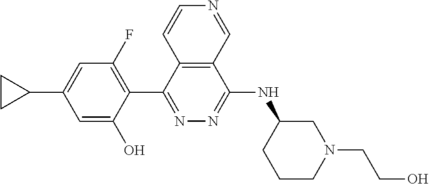 [SMILES: OCCN1CCC[C@@H](Nc2nnc(-c3c(O)cc(C4CC4)cc3F)c3ccncc23)C1] | ***** |
| I-285 | 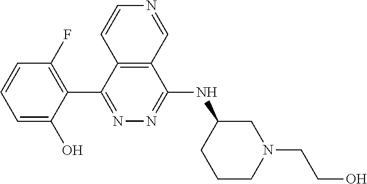 [SMILES: OCCN1CCC[C@@H](Nc2nnc(-c3c(O)cccc3F)c3ccncc23)C1] | **** |
| I-286 | 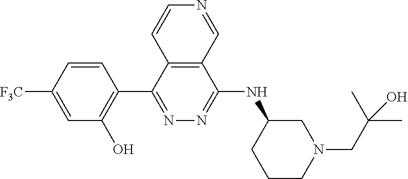 [SMILES: CC(C)(O)CN1CCC[C@@H](Nc2nnc(-c3ccc(C(F)(F)F)cc3O)c3ccncc23)C1] | ***** |
| I-287 | 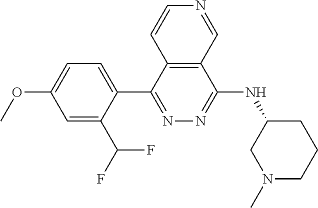 [SMILES: COc1ccc(-c2nnc(N[C@@H]3CCCN(C)C3)c3cnccc23)c(C(F)F)c1] | **** |
| I-288 | 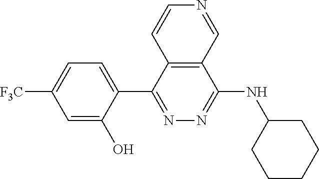 [SMILES: Oc1cc(C(F)(F)F)ccc1-c1nnc(NC2CCCCC2)c2cnccc12] | **** |
| I-289 | 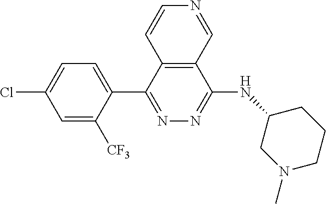 [SMILES: CN1CCC[C@@H](Nc2nnc(-c3ccc(Cl)cc3C(F)(F)F)c3ccncc23)C1] | **** |
| I-290 | 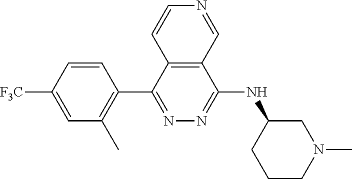 [SMILES: Cc1cc(C(F)(F)F)ccc1-c1nnc(N[C@@H]2CCCN(C)C2)c2cnccc12] | **** |
| I-291 | 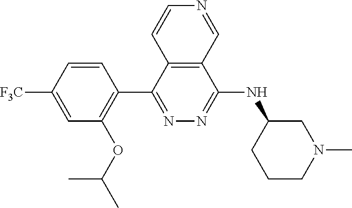 [SMILES: CC(C)Oc1cc(C(F)(F)F)ccc1-c1nnc(N[C@@H]2CCCN(C)C2)c2cnccc12] | **** |
| I-292 | 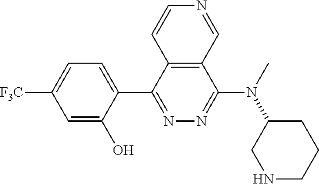 | **** |
| I-293 | 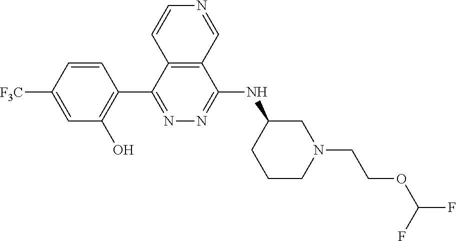 [SMILES: Oc1cc(C(F)(F)F)ccc1-c1nnc(N[C@@H]2CCCN(CCOC(F)F)C2)c2cnccc12] | ***** |
| I-294 | 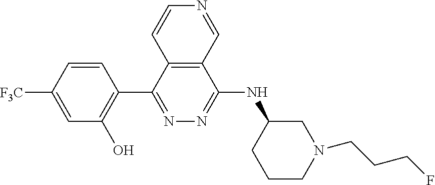 [SMILES: Oc1cc(C(F)(F)F)ccc1-c1nnc(N[C@@H]2CCCN(CCCF)C2)c2cnccc12] | ***** |
| I-295 | 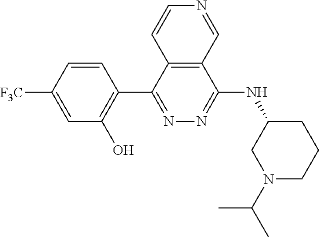 [SMILES: CC(C)N1CCC[C@@H](Nc2nnc(-c3ccc(C(F)(F)F)cc3O)c3ccncc23)C1] | ***** |
| I-296 | 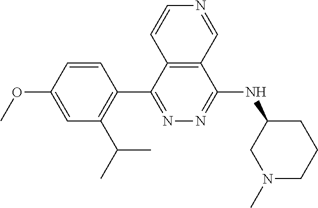 [SMILES: COc1ccc(-c2nnc(N[C@H]3CCCN(C)C3)c3cnccc23)c(C(C)C)c1] | **** |
| I-297 | 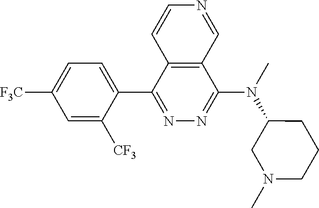 [SMILES: CN1CCC[C@@H](N(C)c2nnc(-c3ccc(C(F)(F)F)cc3C(F)(F)F)c3ccncc23)C1] | ***** |
| I-298 | 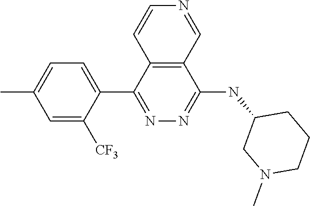 [SMILES: Cc1ccc(-c2nnc(N[C@@H]3CCCN(C)C3)c3cnccc23)c(C(F)(F)F)c1] | **** |
| I-299 | 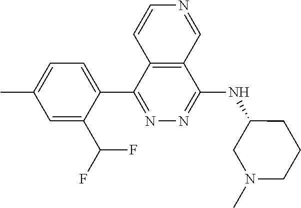 [SMILES: Cc1ccc(-c2nnc(N[C@@H]3CCCN(C)C3)c3cnccc23)c(C(F)F)c1] | ***** |
| I-300 | 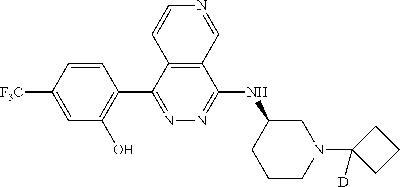 [SMILES: [2H]C1(N2CCC[C@@H](Nc3nnc(-c4ccc(C(F)(F)F)cc4O)c4ccncc34)C2)CCC1] | ***** |
| I-301 | 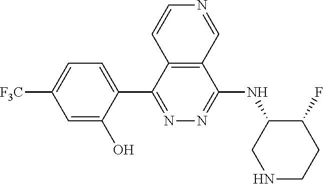 [SMILES: Oc1cc(C(F)(F)F)ccc1-c1nnc(N[C@H]2CNCC[C@H]2F)c2cnccc12] | **** |
| I-302 | 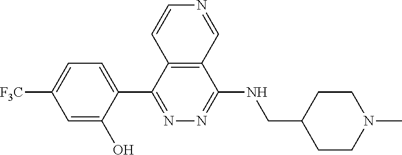 [SMILES: CN1CCC(CNc2nnc(-c3ccc(C(F)(F)F)cc3O)c3ccncc23)CC1] | **** |
| I-303 | 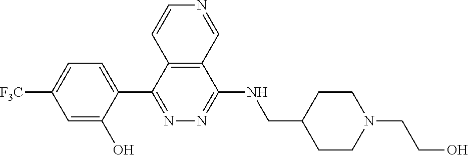 [SMILES: OCCN1CCC(CNc2nnc(-c3ccc(C(F)(F)F)cc3O)c3ccncc23)CC1] | **** |
| I-304 | 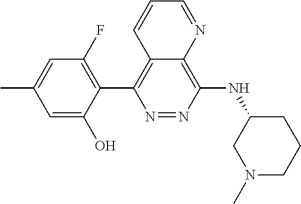 [SMILES: Cc1cc(O)c(-c2nnc(N[C@@H]3CCCN(C)C3)c3ncccc23)c(F)c1] | ***** |
| I-305 | 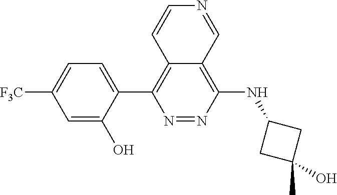 [SMILES: C[C@]1(O)C[C@@H](Nc2nnc(-c3ccc(C(F)(F)F)cc3O)c3ccncc23)C1] | ***** |
| I-306 | 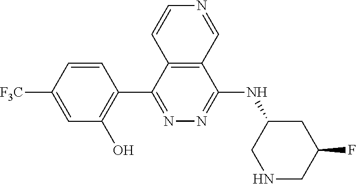 [SMILES: Oc1cc(C(F)(F)F)ccc1-c1nnc(N[C@H]2CNC[C@H](F)C2)c2cnccc12] | ***** |
| I-307 | 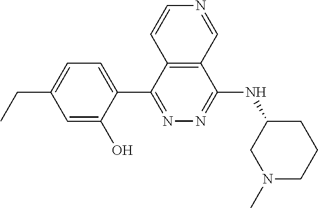 [SMILES: CCc1ccc(-c2nnc(N[C@@H]3CCCN(C)C3)c3cnccc23)c(O)c1] | ***** |
| I-308 | 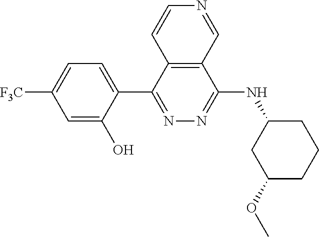 [SMILES: CO[C@H]1CCC[C@@H](Nc2nnc(-c3ccc(C(F)(F)F)cc3O)c3ccncc23)C1] | ***** |
| I-309 | 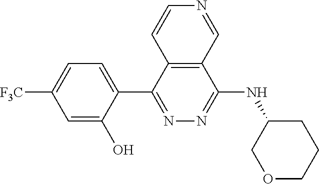 [SMILES: Oc1cc(C(F)(F)F)ccc1-c1nnc(N[C@@H]2CCCOC2)c2cnccc12] | **** |
| I-310 | 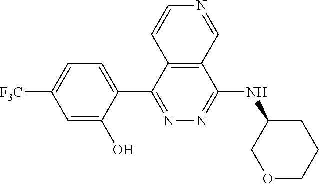 [SMILES: Oc1cc(C(F)(F)F)ccc1-c1nnc(N[C@H]2CCCOC2)c2cnccc12] | **** |
| I-311 | 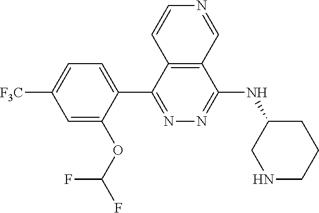 [SMILES: FC(F)Oc1cc(C(F)(F)F)ccc1-c1nnc(N[C@@H]2CCCNC2)c2cnccc12] | **** |
| I-312 | 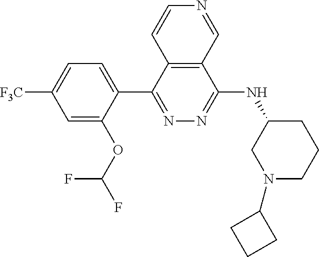 [SMILES: FC(F)Oc1cc(C(F)(F)F)ccc1-c1nnc(N[C@@H]2CCCN(C3CCC3)C2)c2cnccc12] | ***** |
| I-313 | 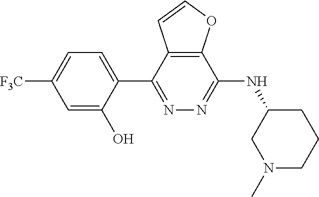 [SMILES: CN1CCC[C@@H](Nc2nnc(-c3ccc(C(F)(F)F)cc3O)c3ccoc23)C1] | ***** |
| I-314 | 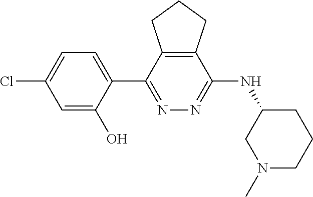 [SMILES: CN1CCC[C@@H](Nc2nnc(-c3ccc(Cl)cc3O)c3c2CCC3)C1] | **** |
| I-315 | 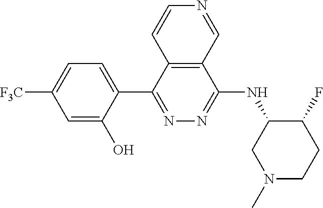 [SMILES: CN1CC[C@@H](F)[C@@H](Nc2nnc(-c3ccc(C(F)(F)F)cc3O)c3ccncc23)C1] | ***** |
| I-316 | 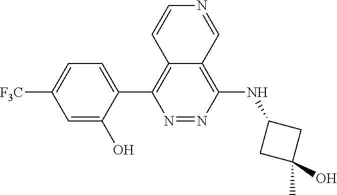 [SMILES: C[C@]1(O)C[C@H](Nc2nnc(-c3ccc(C(F)(F)F)cc3O)c3ccncc23)C1] | **** |
| I-317 | 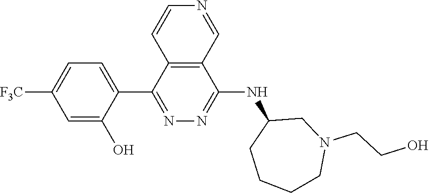 [SMILES: OCCN1CCCC[C@@H](Nc2nnc(-c3ccc(C(F)(F)F)cc3O)c3ccncc23)C1] | **** |
| I-318 | 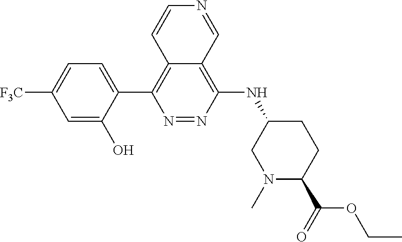 [SMILES: CCOC(=O)[C@@H]1CC[C@@H](Nc2nnc(-c3ccc(C(F)(F)F)cc3O)c3ccncc23)CN1C] | ***** |
| I-319 | 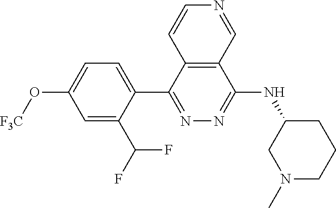 [SMILES: CN1CCC[C@@H](Nc2nnc(-c3ccc(OC(F)(F)F)cc3C(F)F)c3ccncc23)C1] | **** |
| I-320 | 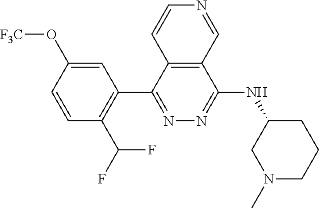 [SMILES: CN1CCC[C@@H](Nc2nnc(-c3cc(OC(F)(F)F)ccc3C(F)F)c3ccncc23)C1] | **** |
| I-321 | 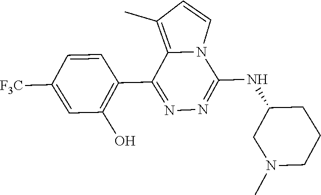 [SMILES: Cc1ccn2c(N[C@@H]3CCCN(C)C3)nnc(-c3ccc(C(F)(F)F)cc3O)c12] | *** |
| I-322 | 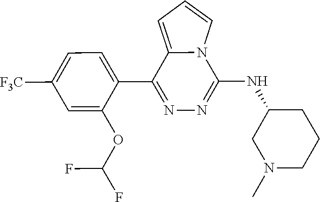 [SMILES: CN1CCC[C@@H](Nc2nnc(-c3ccc(C(F)(F)F)cc3OC(F)F)c3cccn23)C1] | ***** |
| I-323 | 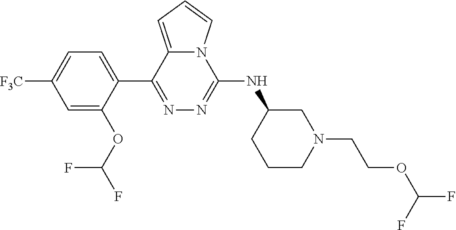 [SMILES: FC(F)OCCN1CCC[C@@H](Nc2nnc(-c3ccc(C(F)(F)F)cc3OC(F)F)c3cccn23)C1] | **** |
| I-324 | 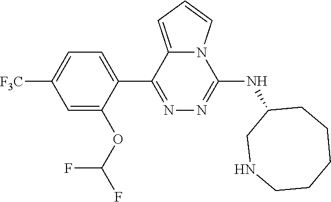 [SMILES: FC(F)Oc1cc(C(F)(F)F)ccc1-c1nnc(N[C@@H]2CCCCCNC2)n2cccc12] | **** |
| I-325 | 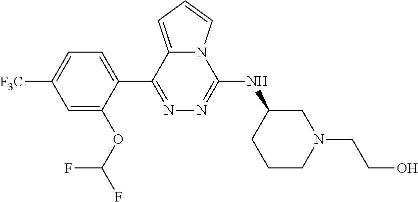 [SMILES: OCCN1CCC[C@@H](Nc2nnc(-c3ccc(C(F)(F)F)cc3OC(F)F)c3cccn23)C1] | **** |
| I-326 | 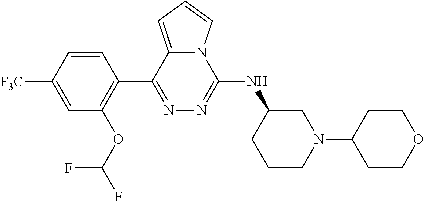 [SMILES: FC(F)Oc1cc(C(F)(F)F)ccc1-c1nnc(N[C@@H]2CCCN(C3CCOCC3)C2)n2cccc12] | ***** |
| I-327 | 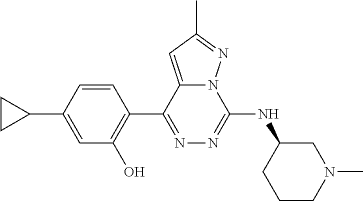 [SMILES: Cc1cc2c(-c3ccc(C4CC4)cc3O)nnc(N[C@@H]3CCCN(C)C3)n2n1] | **** |
| I-328 | 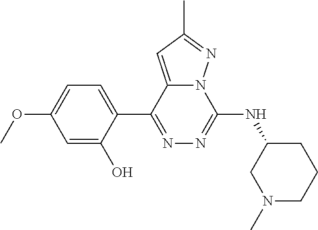 [SMILES: COc1ccc(-c2nnc(N[C@@H]3CCCN(C)C3)n3nc(C)cc23)c(O)c1] | ***** |
| I-329 | 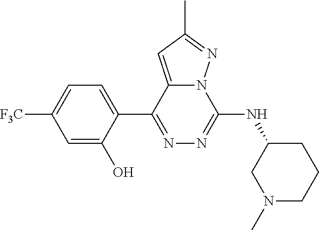 [SMILES: Cc1cc2c(-c3ccc(C(F)(F)F)cc3O)nnc(N[C@@H]3CCCN(C)C3)n2n1] | ***** |
| I-330 | 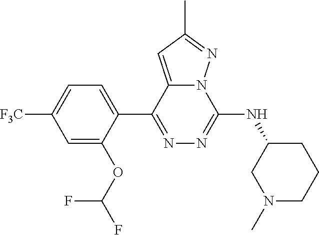 [SMILES: Cc1cc2c(-c3ccc(C(F)(F)F)cc3OC(F)F)nnc(N[C@@H]3CCCN(C)C3)n2n1] | **** |
| I-331 | 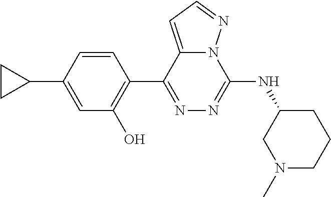 [SMILES: CN1CCC[C@@H](Nc2nnc(-c3ccc(C4CC4)cc3O)c3ccnn23)C1] | **** |
| I-332 | 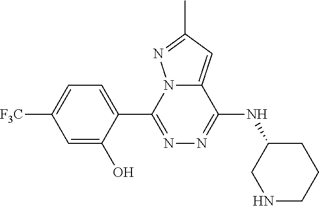 [SMILES: Cc1cc2c(N[C@@H]3CCCNC3)nnc(-c3ccc(C(F)(F)F)cc3O)n2n1] | *** |
| I-333 | 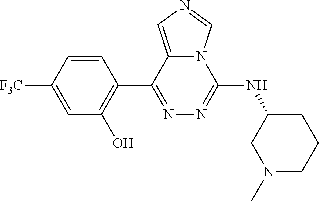 [SMILES: CN1CCC[C@@H](Nc2nnc(-c3ccc(C(F)(F)F)cc3O)c3cncn23)C1] | ***** |
| I-334 | 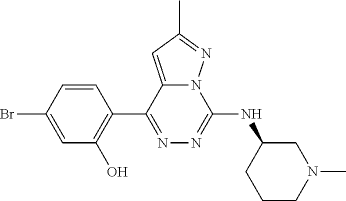 [SMILES: Cc1cc2c(-c3ccc(Br)cc3O)nnc(N[C@@H]3CCCN(C)C3)n2n1] | ***** |
| I-335 | 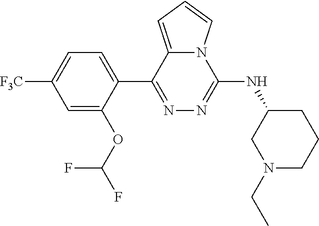 [SMILES: CCN1CCC[C@@H](Nc2nnc(-c3ccc(C(F)(F)F)cc3OC(F)F)c3cccn23)C1] | ***** |
| I-336 | 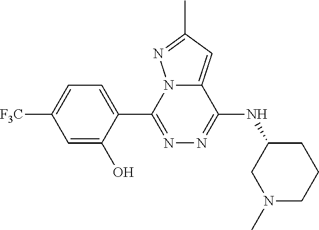 [SMILES: Cc1cc2c(N[C@@H]3CCCN(C)C3)nnc(-c3ccc(C(F)(F)F)cc3O)n2n1] | *** |
| I-337 | 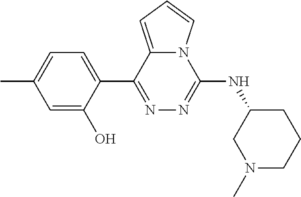 [SMILES: Cc1ccc(-c2nnc(N[C@@H]3CCCN(C)C3)n3cccc23)c(O)c1] | **** |
| I-338 | 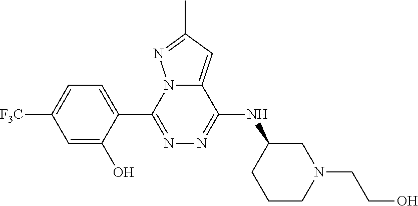 [SMILES: Cc1cc2c(N[C@@H]3CCCN(CCO)C3)nnc(-c3ccc(C(F)(F)F)cc3O)n2n1] | *** |
| I-339 | 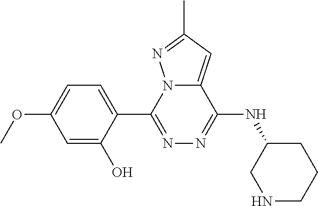 [SMILES: COc1ccc(-c2nnc(N[C@@H]3CCCNC3)c3cc(C)nn23)c(O)c1] | ** |
| I-340 | 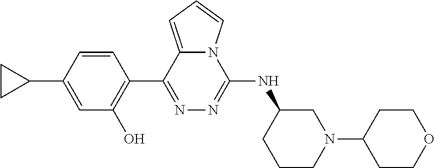 [SMILES: Oc1cc(C2CC2)ccc1-c1nnc(N[C@@H]2CCCN(C3CCOCC3)C2)n2cccc12] | **** |
| I-341 | 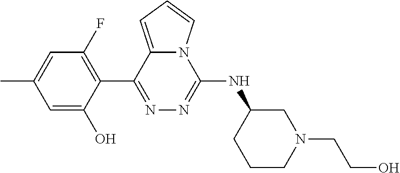 [SMILES: Cc1cc(O)c(-c2nnc(N[C@@H]3CCCN(CCO)C3)n3cccc23)c(F)c1] | **** |
| I-342 | 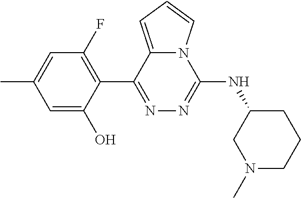 [SMILES: Cc1cc(O)c(-c2nnc(N[C@@H]3CCCN(C)C3)n3cccc23)c(F)c1] | **** |
| I-343 | 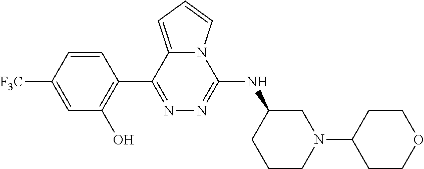 [SMILES: Oc1cc(C(F)(F)F)ccc1-c1nnc(N[C@@H]2CCCN(C3CCOCC3)C2)n2cccc12] | **** |
| I-344 | 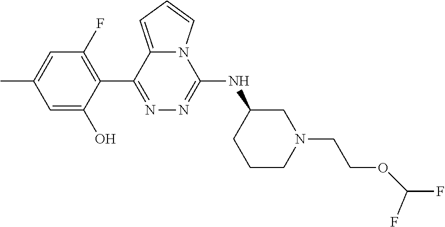 [SMILES: Cc1cc(O)c(-c2nnc(N[C@@H]3CCCN(CCOC(F)F)C3)n3cccc23)c(F)c1] | ***** |
| I-345 | 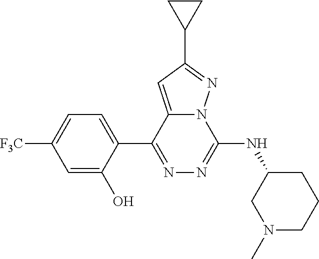 [SMILES: CN1CCC[C@@H](Nc2nnc(-c3ccc(C(F)(F)F)cc3O)c3cc(C4CC4)nn23)C1] | **** |
| I-346 | 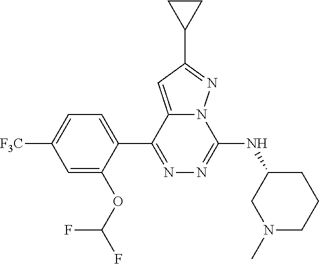 [SMILES: CN1CCC[C@@H](Nc2nnc(-c3ccc(C(F)(F)F)cc3OC(F)F)c3cc(C4CC4)nn23)C1] | **** |
| I-347 | 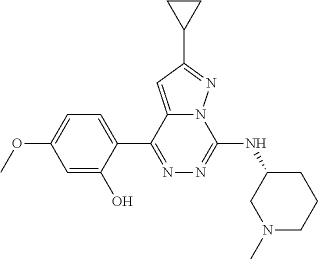 [SMILES: COc1ccc(-c2nnc(N[C@@H]3CCCN(C)C3)n3nc(C4CC4)cc23)c(O)c1] | *** |
| I-348 | 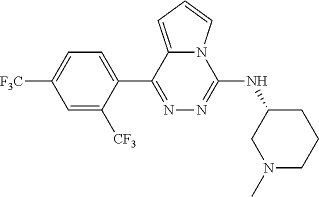 [SMILES: CN1CCC[C@@H](Nc2nnc(-c3ccc(C(F)(F)F)cc3C(F)(F)F)c3cccn23)C1] | **** |
| I-349 | 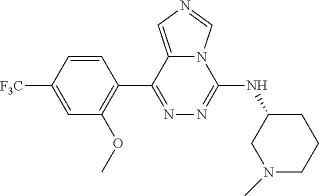 [SMILES: COc1cc(C(F)(F)F)ccc1-c1nnc(N[C@@H]2CCCN(C)C2)n2cncc12] | **** |
| I-350 | 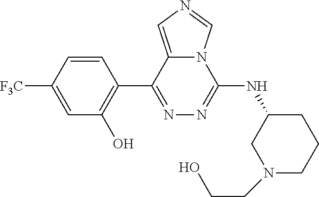 [SMILES: OCCN1CCC[C@@H](Nc2nnc(-c3ccc(C(F)(F)F)cc3O)c3cncn23)C1] | **** |
| I-351 | 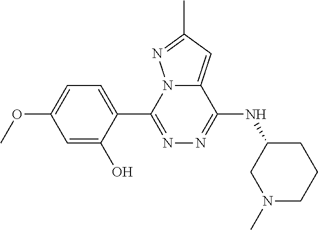 [SMILES: COc1ccc(-c2nnc(N[C@@H]3CCCN(C)C3)c3cc(C)nn23)c(O)c1] | *** |
| I-352 | 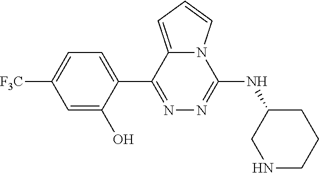 [SMILES: Oc1cc(C(F)(F)F)ccc1-c1nnc(N[C@@H]2CCCNC2)n2cccc12] | **** |
| I-353 | 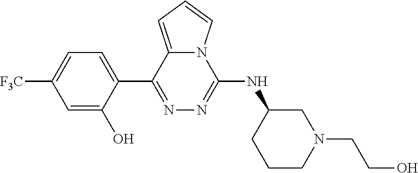 [SMILES: OCCN1CCC[C@@H](Nc2nnc(-c3ccc(C(F)(F)F)cc3O)c3cccn23)C1] | **** |
| I-354 | 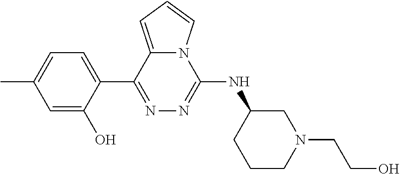 [SMILES: Cc1ccc(-c2nnc(N[C@@H]3CCCN(CCO)C3)n3cccc23)c(O)c1] | ***** |
| I-355 | 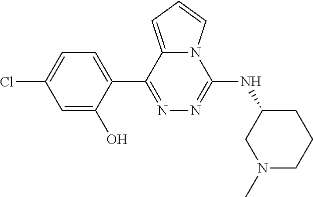 [SMILES: CN1CCC[C@@H](Nc2nnc(-c3ccc(Cl)cc3O)c3cccn23)C1] | ***** |
| I-356 | 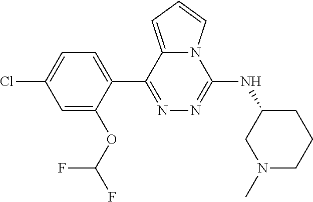 [SMILES: CN1CCC[C@@H](Nc2nnc(-c3ccc(Cl)cc3OC(F)F)c3cccn23)C1] | **** |
| I-357 | 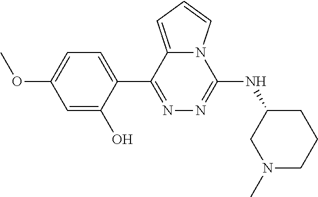 [SMILES: COc1ccc(-c2nnc(N[C@@H]3CCCN(C)C3)n3cccc23)c(O)c1] | **** |
| I-358 | 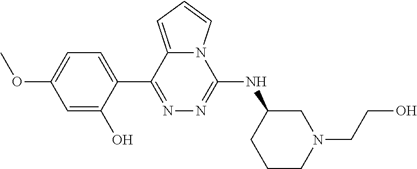 [SMILES: COc1ccc(-c2nnc(N[C@@H]3CCCN(CCO)C3)n3cccc23)c(O)c1] | **** |
| I-359 | 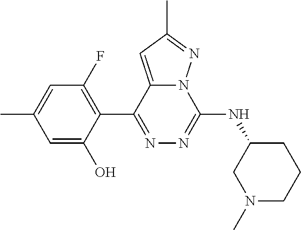 [SMILES: Cc1cc(O)c(-c2nnc(N[C@@H]3CCCN(C)C3)n3nc(C)cc23)c(F)c1] | ***** |
| I-360 | 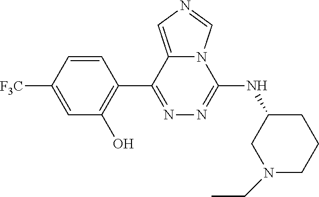 [SMILES: CCN1CCC[C@@H](Nc2nnc(-c3ccc(C(F)(F)F)cc3O)c3cncn23)C1] | ***** |
| I-361 | 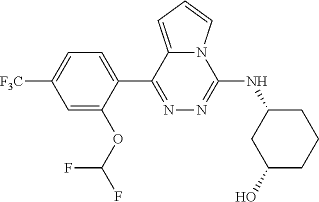 [SMILES: O[C@H]1CCC[C@@H](Nc2nnc(-c3ccc(C(F)(F)F)cc3OC(F)F)c3cccn23)C1] | **** |
| I-362 | 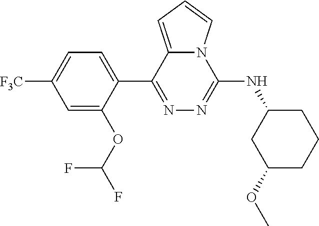 [SMILES: CO[C@H]1CCC[C@@H](Nc2nnc(-c3ccc(C(F)(F)F)cc3OC(F)F)c3cccn23)C1] | *** |
| I-363 | 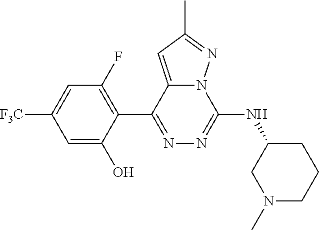 [SMILES: Cc1cc2c(-c3c(O)cc(C(F)(F)F)cc3F)nnc(N[C@@H]3CCCN(C)C3)n2n1] | ***** |
| I-364 | 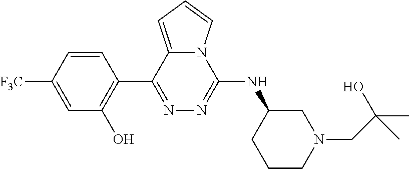 [SMILES: CC(C)(O)CN1CCC[C@@H](Nc2nnc(-c3ccc(C(F)(F)F)cc3O)c3cccn23)C1] | **** |
| I-365 | 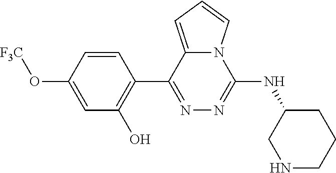 [SMILES: Oc1cc(OC(F)(F)F)ccc1-c1nnc(N[C@@H]2CCCNC2)n2cccc12] | *** |
| I-366 | 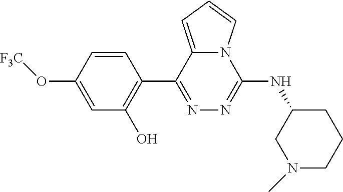 [SMILES: CN1CCC[C@@H](Nc2nnc(-c3ccc(OC(F)(F)F)cc3O)c3cccn23)C1] | **** |
| I-367 | 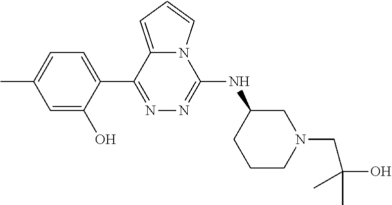 [SMILES: Cc1ccc(-c2nnc(N[C@@H]3CCCN(CC(C)(C)O)C3)n3cccc23)c(O)c1] | *** |
| I-368 | 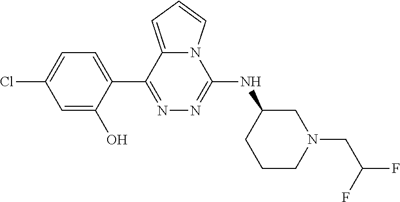 [SMILES: Oc1cc(Cl)ccc1-c1nnc(N[C@@H]2CCCN(CC(F)F)C2)n2cccc12] | *** |
| I-369 | 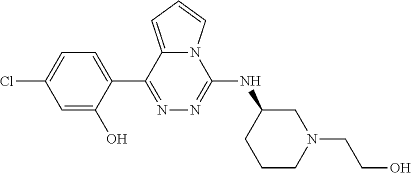 [SMILES: OCCN1CCC[C@@H](Nc2nnc(-c3ccc(Cl)cc3O)c3cccn23)C1] | **** |
| I-370 | 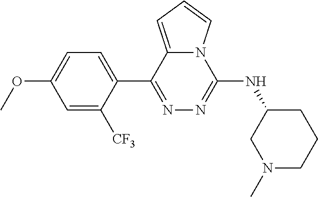 [SMILES: COc1ccc(-c2nnc(N[C@@H]3CCCN(C)C3)n3cccc23)c(C(F)(F)F)c1] | **** |
| I-371 | 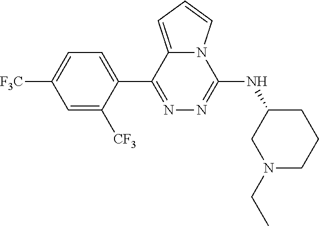 [SMILES: CCN1CCC[C@@H](Nc2nnc(-c3ccc(C(F)(F)F)cc3C(F)(F)F)c3cccn23)C1] | ***** |
| I-372 | 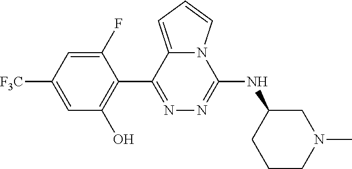 [SMILES: CN1CCC[C@@H](Nc2nnc(-c3c(O)cc(C(F)(F)F)cc3F)c3cccn23)C1] | ***** |
| I-373 | 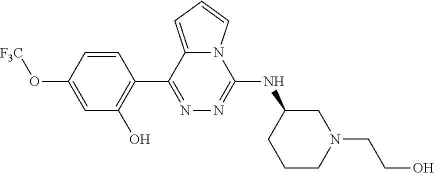 [SMILES: OCCN1CCC[C@@H](Nc2nnc(-c3ccc(OC(F)(F)F)cc3O)c3cccn23)C1] | **** |
| I-374 | 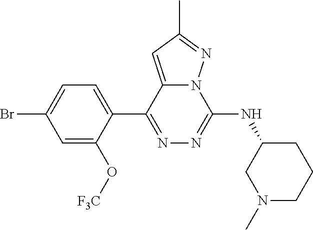 [SMILES: Cc1cc2c(-c3ccc(Br)cc3OC(F)(F)F)nnc(N[C@@H]3CCCN(C)C3)n2n1] | **** |
| I-375 | 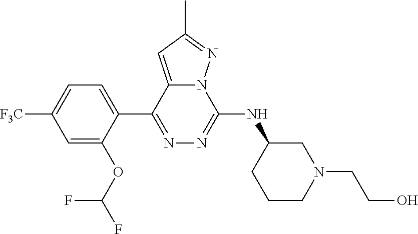 [SMILES: Cc1cc2c(-c3ccc(C(F)(F)F)cc3OC(F)F)nnc(N[C@@H]3CCCN(CCO)C3)n2n1] | **** |
| I-376 | 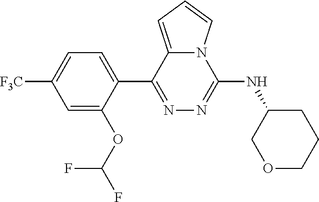 [SMILES: FC(F)Oc1cc(C(F)(F)F)ccc1-c1nnc(N[C@@H]2CCCOC2)n2cccc12] | **** |
| I-377 | 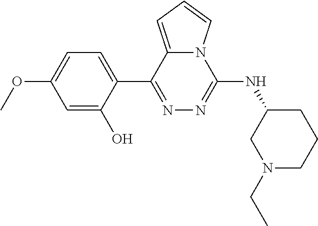 [SMILES: CCN1CCC[C@@H](Nc2nnc(-c3ccc(OC)cc3O)c3cccn23)C1] | **** |
| I-378 | 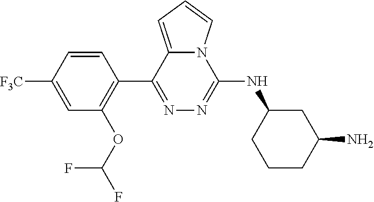 [SMILES: N[C@H]1CCC[C@@H](Nc2nnc(-c3ccc(C(F)(F)F)cc3OC(F)F)c3cccn23)C1] | *** |
| I-379 | 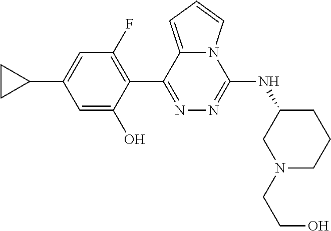 [SMILES: OCCN1CCC[C@@H](Nc2nnc(-c3c(O)cc(C4CC4)cc3F)c3cccn23)C1] | **** |
| I-380 | 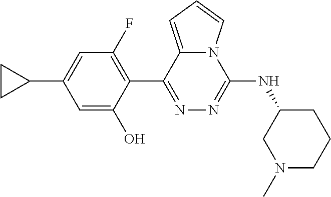 [SMILES: CN1CCC[C@@H](Nc2nnc(-c3c(O)cc(C4CC4)cc3F)c3cccn23)C1] | ***** |
| I-381 | 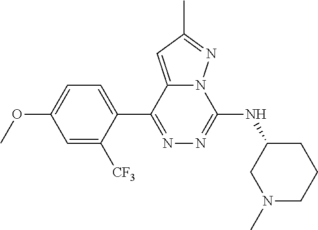 [SMILES: COc1ccc(-c2nnc(N[C@@H]3CCCN(C)C3)n3nc(C)cc23)c(C(F)(F)F)c1] | **** |
| I-382 | 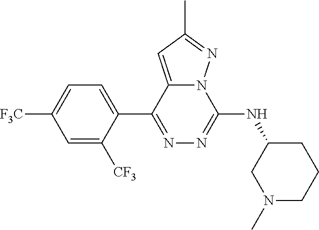 [SMILES: Cc1cc2c(-c3ccc(C(F)(F)F)cc3C(F)(F)F)nnc(N[C@@H]3CCCN(C)C3)n2n1] | **** |
| I-383 | 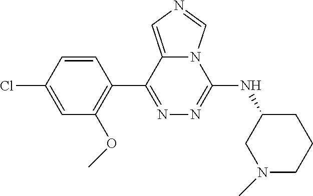 [SMILES: COc1cc(Cl)ccc1-c1nnc(N[C@@H]2CCCN(C)C2)n2cncc12] | **** |
| I-384 | 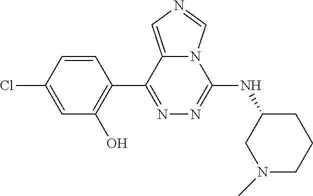 [SMILES: CN1CCC[C@@H](Nc2nnc(-c3ccc(Cl)cc3O)c3cncn23)C1] | ***** |
| I-385 | 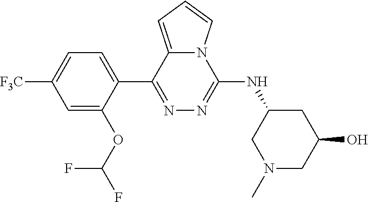 [SMILES: CN1C[C@H](O)C[C@@H](Nc2nnc(-c3ccc(C(F)(F)F)cc3OC(F)F)c3cccn23)C1] | ** |
| I-386 | 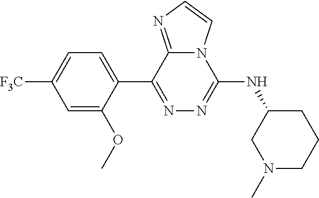 [SMILES: COc1cc(C(F)(F)F)ccc1-c1nnc(N[C@@H]2CCCN(C)C2)n2ccnc12] | ** |
| I-387 | 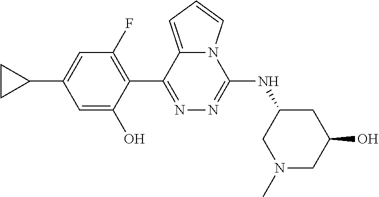 [SMILES: CN1C[C@H](O)C[C@@H](Nc2nnc(-c3c(O)cc(C4CC4)cc3F)c3cccn23)C1] | *** |
| I-388 | 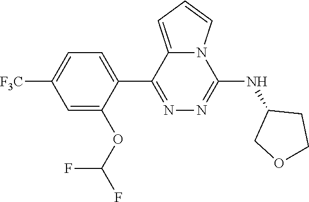 [SMILES: FC(F)Oc1cc(C(F)(F)F)ccc1-c1nnc(N[C@@H]2CCOC2)n2cccc12] | **** |
| I-389 | 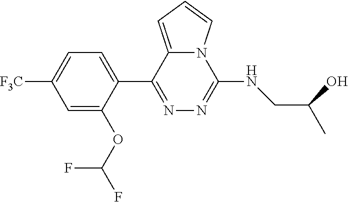 [SMILES: C[C@H](O)CNc1nnc(-c2ccc(C(F)(F)F)cc2OC(F)F)c2cccn12] | **** |
| I-390 | 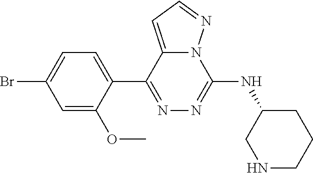 [SMILES: COc1cc(Br)ccc1-c1nnc(N[C@@H]2CCCNC2)n2nccc12] | ** |
| I-391 | 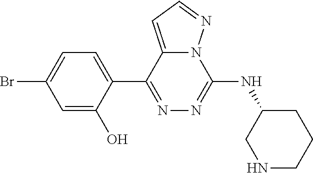 [SMILES: Oc1cc(Br)ccc1-c1nnc(N[C@@H]2CCCNC2)n2nccc12] | **** |
| I-392 | 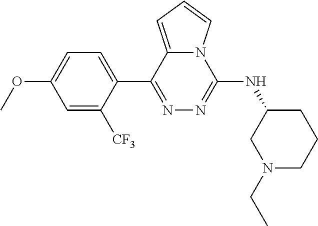 [SMILES: CCN1CCC[C@@H](Nc2nnc(-c3ccc(OC)cc3C(F)(F)F)c3cccn23)C1] | **** |
| I-393 | 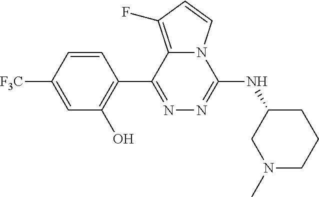 [SMILES: CN1CCC[C@@H](Nc2nnc(-c3ccc(C(F)(F)F)cc3O)c3c(F)ccn23)C1] | ***** |
| I-394 | 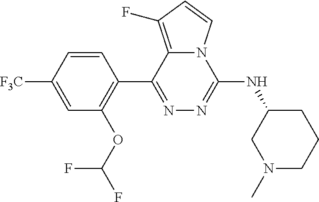 [SMILES: CN1CCC[C@@H](Nc2nnc(-c3ccc(C(F)(F)F)cc3OC(F)F)c3c(F)ccn23)C1] | **** |
| I-395 | 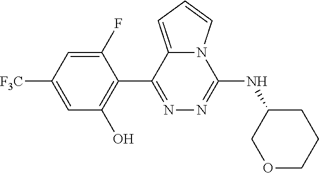 [SMILES: Oc1cc(C(F)(F)F)cc(F)c1-c1nnc(N[C@@H]2CCCOC2)n2cccc12] | *** |
| I-396 | 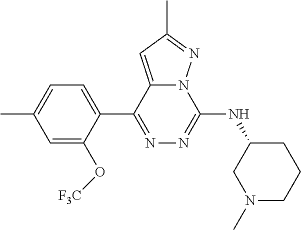 [SMILES: Cc1ccc(-c2nnc(N[C@@H]3CCCN(C)C3)n3nc(C)cc23)c(OC(F)(F)F)c1] | *** |
| I-397 | 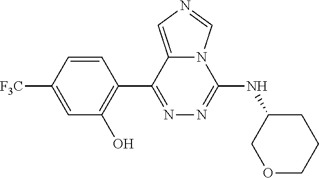 [SMILES: Oc1cc(C(F)(F)F)ccc1-c1nnc(N[C@@H]2CCCOC2)n2cncc12] | *** |
| I-398 | 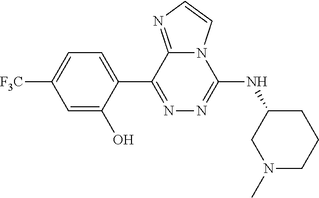 [SMILES: CN1CCC[C@@H](Nc2nnc(-c3ccc(C(F)(F)F)cc3O)c3nccn23)C1] | * |
| I-399 | 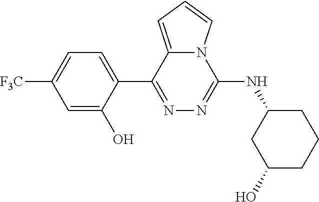 [SMILES: Oc1cc(C(F)(F)F)ccc1-c1nnc(N[C@@H]2CCC[C@H](O)C2)n2cccc12] | *** |
| I-400 | 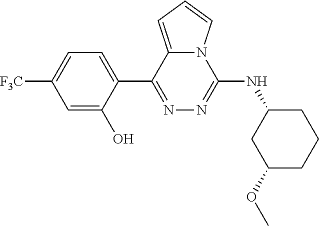 [SMILES: CO[C@H]1CCC[C@@H](Nc2nnc(-c3ccc(C(F)(F)F)cc3O)c3cccn23)C1] | ** |
| I-401 | 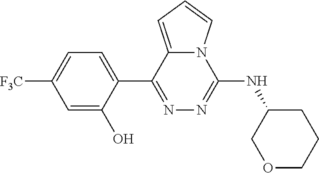 [SMILES: Oc1cc(C(F)(F)F)ccc1-c1nnc(N[C@@H]2CCCOC2)n2cccc12] | ** |
| I-402 | 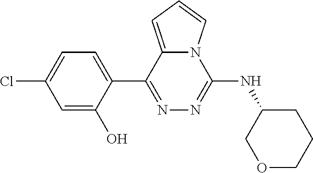 [SMILES: Oc1cc(Cl)ccc1-c1nnc(N[C@@H]2CCCOC2)n2cccc12] | *** |
| I-403 | 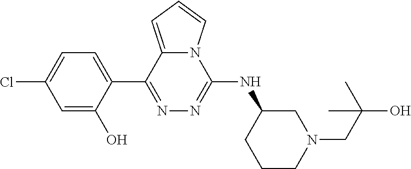 [SMILES: CC(C)(O)CN1CCC[C@@H](Nc2nnc(-c3ccc(Cl)cc3O)c3cccn23)C1] | *** |
| I-404 | 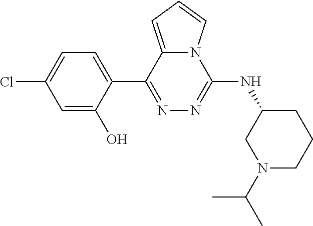 [SMILES: CC(C)N1CCC[C@@H](Nc2nnc(-c3ccc(Cl)cc3O)c3cccn23)C1] | ***** |
| I-405 | 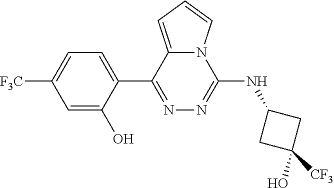 [SMILES: Oc1cc(C(F)(F)F)ccc1-c1nnc(N[C@H]2C[C@](O)(C(F)(F)F)C2)n2cccc12] | ** |
| I-406 | 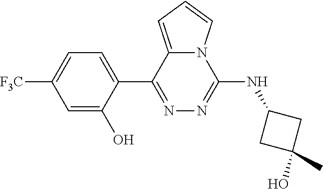 [SMILES: C[C@]1(O)C[C@@H](Nc2nnc(-c3ccc(C(F)(F)F)cc3O)c3cccn23)C1] | **** |
| I-407 | 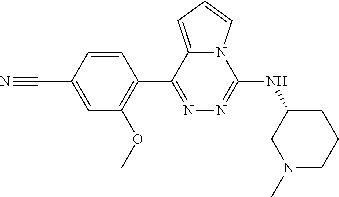 [SMILES: COc1cc(C#N)ccc1-c1nnc(N[C@@H]2CCCN(C)C2)n2cccc12] | *** |
| I-408 | 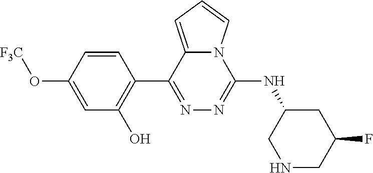 [SMILES: Oc1cc(OC(F)(F)F)ccc1-c1nnc(N[C@H]2CNC[C@H](F)C2)n2cccc12] | *** |
| I-409 | 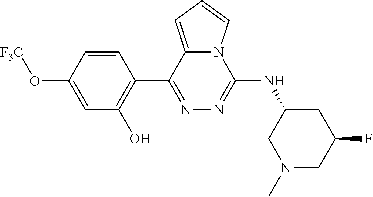 [SMILES: CN1C[C@H](F)C[C@@H](Nc2nnc(-c3ccc(OC(F)(F)F)cc3O)c3cccn23)C1] | **** |
| I-410 | 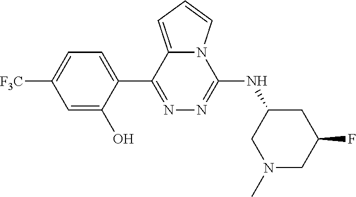 [SMILES: CN1C[C@H](F)C[C@@H](Nc2nnc(-c3ccc(C(F)(F)F)cc3O)c3cccn23)C1] | **** |
| I-411 | 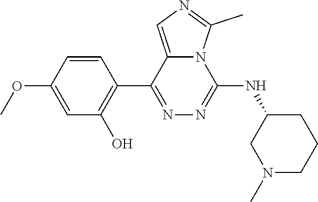 [SMILES: COc1ccc(-c2nnc(N[C@@H]3CCCN(C)C3)n3c(C)ncc23)c(O)c1] | *** |
| I-412 | 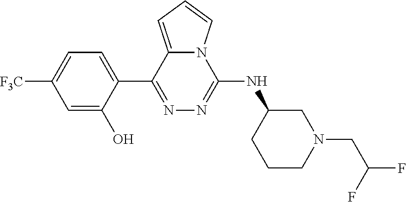 [SMILES: Oc1cc(C(F)(F)F)ccc1-c1nnc(N[C@@H]2CCCN(CC(F)F)C2)n2cccc12] | ***** |
| I-413 | 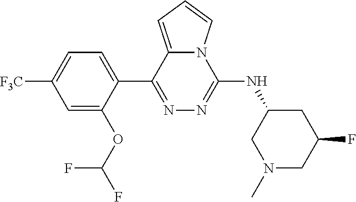 [SMILES: CN1C[C@H](F)C[C@@H](Nc2nnc(-c3ccc(C(F)(F)F)cc3OC(F)F)c3cccn23)C1] | **** |
| I-414 | 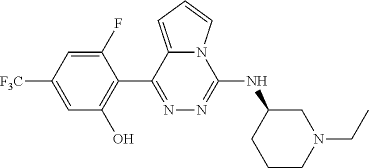 [SMILES: CCN1CCC[C@@H](Nc2nnc(-c3c(O)cc(C(F)(F)F)cc3F)c3cccn23)C1] | ***** |
| I-415 | 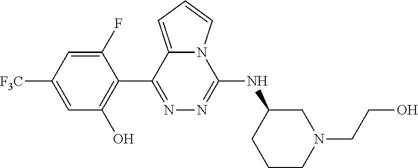 [SMILES: OCCN1CCC[C@@H](Nc2nnc(-c3c(O)cc(C(F)(F)F)cc3F)c3cccn23)C1] | **** |
| I-416 | 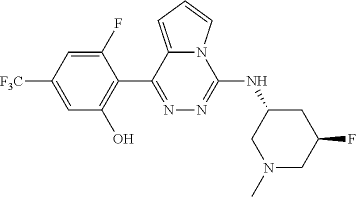 [SMILES: CN1C[C@H](F)C[C@@H](Nc2nnc(-c3c(O)cc(C(F)(F)F)cc3F)c3cccn23)C1] | **** |
| I-417 | 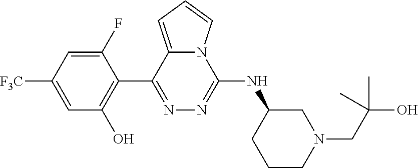 [SMILES: CC(C)(O)CN1CCC[C@@H](Nc2nnc(-c3c(O)cc(C(F)(F)F)cc3F)c3cccn23)C1] | **** |
| I-418 | 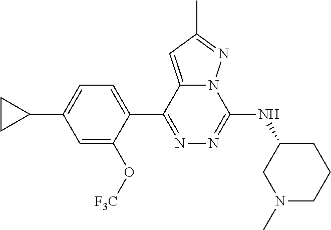 [SMILES: Cc1cc2c(-c3ccc(C4CC4)cc3OC(F)(F)F)nnc(N[C@@H]3CCCN(C)C3)n2n1] | **** |
| I-419 | 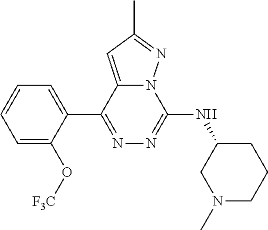 [SMILES: Cc1cc2c(-c3ccccc3OC(F)(F)F)nnc(N[C@@H]3CCCN(C)C3)n2n1] | *** |
| I-420 | 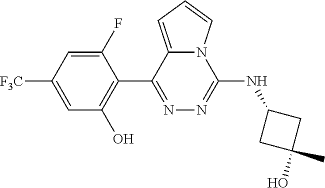 [SMILES: C[C@]1(O)C[C@@H](Nc2nnc(-c3c(O)cc(C(F)(F)F)cc3F)c3cccn23)C1] | **** |
| I-421 | 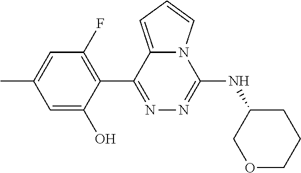 [SMILES: Cc1cc(O)c(-c2nnc(N[C@@H]3CCCOC3)n3cccc23)c(F)c1] | **** |
| I-422 | 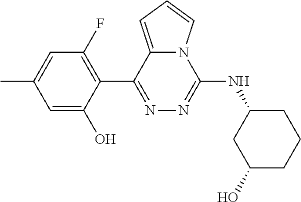 [SMILES: Cc1cc(O)c(-c2nnc(N[C@@H]3CCC[C@H](O)C3)n3cccc23)c(F)c1] | **** |
| I-423 | 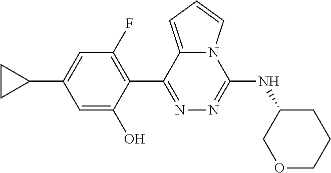 [SMILES: Oc1cc(C2CC2)cc(F)c1-c1nnc(N[C@@H]2CCCOC2)n2cccc12] | *** |
| I-424 | 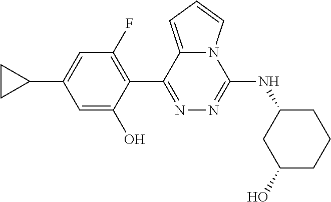 [SMILES: Oc1cc(C2CC2)cc(F)c1-c1nnc(N[C@@H]2CCC[C@H](O)C2)n2cccc12] | **** |
| I-425 | 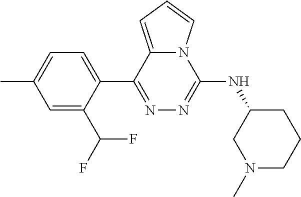 [SMILES: Cc1ccc(-c2nnc(N[C@@H]3CCCN(C)C3)n3cccc23)c(C(F)F)c1] | *** |
| I-426 | 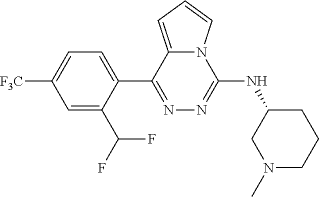 [SMILES: CN1CCC[C@@H](Nc2nnc(-c3ccc(C(F)(F)F)cc3C(F)F)c3cccn23)C1] | *** |
| I-427 | 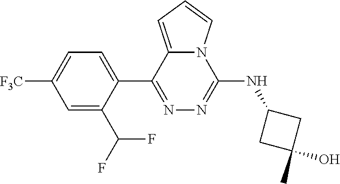 [SMILES: C[C@]1(O)C[C@@H](Nc2nnc(-c3ccc(C(F)(F)F)cc3C(F)F)c3cccn23)C1] | *** |
| I-428 | 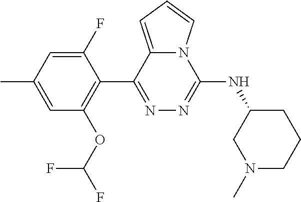 [SMILES: Cc1cc(F)c(-c2nnc(N[C@@H]3CCCN(C)C3)n3cccc23)c(OC(F)F)c1] | *** |
| I-429 | 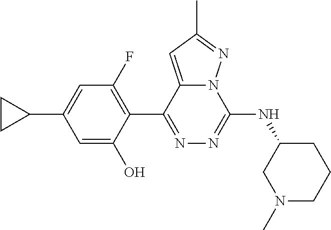 [SMILES: Cc1cc2c(-c3c(O)cc(C4CC4)cc3F)nnc(N[C@@H]3CCCN(C)C3)n2n1] | ***** |
| I-430 | 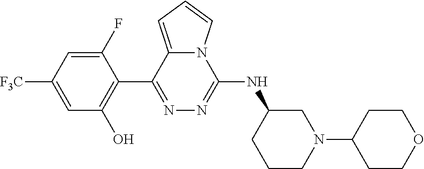 [SMILES: Oc1cc(C(F)(F)F)cc(F)c1-c1nnc(N[C@@H]2CCCN(C3CCOCC3)C2)n2cccc12] | **** |
| I-431 | 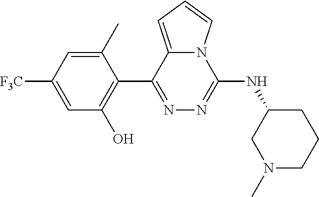 [SMILES: Cc1cc(C(F)(F)F)cc(O)c1-c1nnc(N[C@@H]2CCCN(C)C2)n2cccc12] | ***** |
| I-432 | 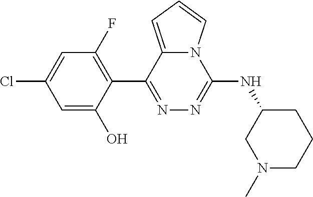 [SMILES: CN1CCC[C@@H](Nc2nnc(-c3c(O)cc(Cl)cc3F)c3cccn23)C1] | ***** |
| I-433 | 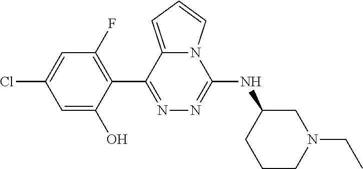 [SMILES: CCN1CCC[C@@H](Nc2nnc(-c3c(O)cc(Cl)cc3F)c3cccn23)C1] | ***** |
| I-434 | 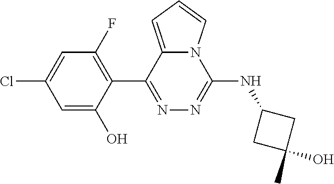 [SMILES: C[C@]1(O)C[C@@H](Nc2nnc(-c3c(O)cc(Cl)cc3F)c3cccn23)C1] | **** |
| I-435 | 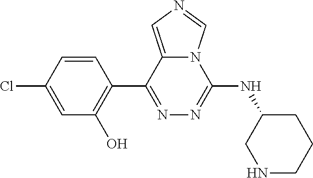 [SMILES: Oc1cc(Cl)ccc1-c1nnc(N[C@@H]2CCCNC2)n2cncc12] | **** |
| I-436 | 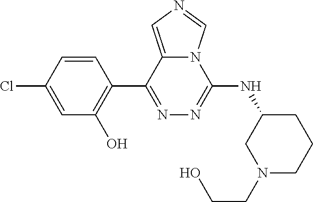 [SMILES: OCCN1CCC[C@@H](Nc2nnc(-c3ccc(Cl)cc3O)c3cncn23)C1] | **** |
| I-437 | 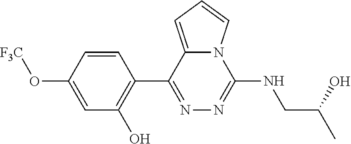 [SMILES: C[C@@H](O)CNc1nnc(-c2ccc(OC(F)(F)F)cc2O)c2cccn12] | *** |
| I-438 | 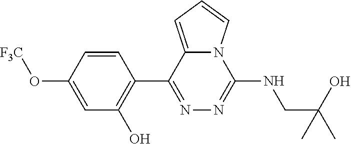 [SMILES: CC(C)(O)CNc1nnc(-c2ccc(OC(F)(F)F)cc2O)c2cccn12] | ** |
| I-439 | 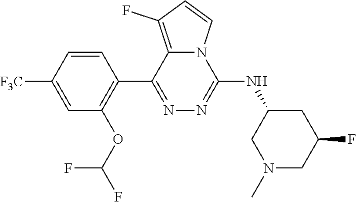 [SMILES: CN1C[C@H](F)C[C@@H](Nc2nnc(-c3ccc(C(F)(F)F)cc3OC(F)F)c3c(F)ccn23)C1] | **** |
| I-440 | 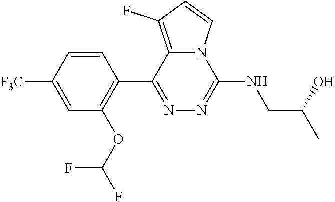 [SMILES: C[C@@H](O)CNc1nnc(-c2ccc(C(F)(F)F)cc2OC(F)F)c2c(F)ccn12] | *** |
| I-441 | 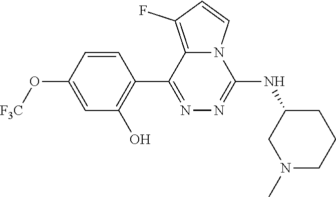 [SMILES: CN1CCC[C@@H](Nc2nnc(-c3ccc(OC(F)(F)F)cc3O)c3c(F)ccn23)C1] | **** |
| I-442 | 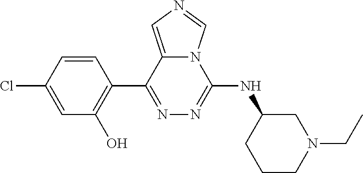 [SMILES: CCN1CCC[C@@H](Nc2nnc(-c3ccc(Cl)cc3O)c3cncn23)C1] | ***** |
| I-443 | 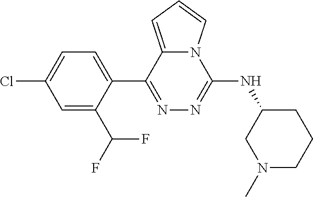 [SMILES: CN1CCC[C@@H](Nc2nnc(-c3ccc(Cl)cc3C(F)F)c3cccn23)C1] | **** |
| I-444 | 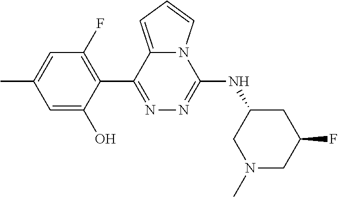 [SMILES: Cc1cc(O)c(-c2nnc(N[C@@H]3C[C@@H](F)CN(C)C3)n3cccc23)c(F)c1] | ***** |
| I-445 | 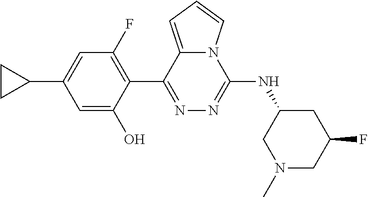 [SMILES: CN1C[C@H](F)C[C@@H](Nc2nnc(-c3c(O)cc(C4CC4)cc3F)c3cccn23)C1] | **** |
| I-446 | 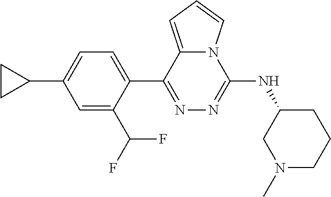 [SMILES: CN1CCC[C@@H](Nc2nnc(-c3ccc(C4CC4)cc3C(F)F)c3cccn23)C1] | **** |
| I-447 | 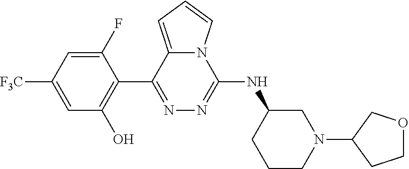 [SMILES: Oc1cc(C(F)(F)F)cc(F)c1-c1nnc(N[C@@H]2CCCN(C3CCOC3)C2)n2cccc12] | **** |
| I-448 | 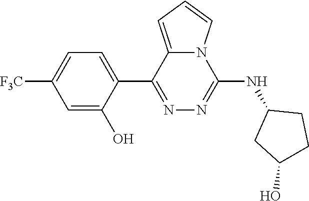 [SMILES: Oc1cc(C(F)(F)F)ccc1-c1nnc(N[C@@H]2CC[C@H](O)C2)n2cccc12] | *** |
| I-449 | 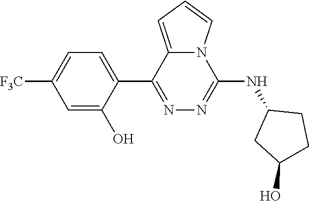 [SMILES: Oc1cc(C(F)(F)F)ccc1-c1nnc(N[C@@H]2CC[C@@H](O)C2)n2cccc12] | *** |
| I-450 | 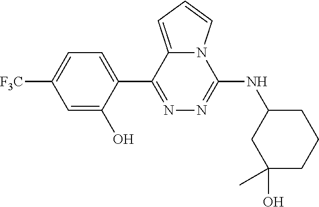 [SMILES: CC1(O)CCCC(Nc2nnc(-c3ccc(C(F)(F)F)cc3O)c3cccn23)C1] | *** |
| I-451 | 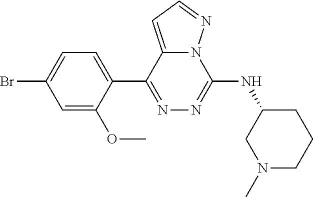 [SMILES: COc1cc(Br)ccc1-c1nnc(N[C@@H]2CCCN(C)C2)n2nccc12] | *** |
| I-452 | 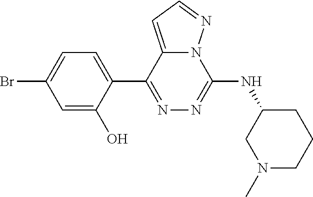 [SMILES: CN1CCC[C@@H](Nc2nnc(-c3ccc(Br)cc3O)c3ccnn23)C1] | **** |
| I-453 | 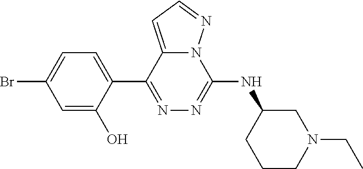 [SMILES: CCN1CCC[C@@H](Nc2nnc(-c3ccc(Br)cc3O)c3ccnn23)C1] | ***** |
| I-454 | 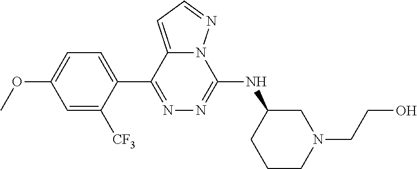 [SMILES: COc1ccc(-c2nnc(N[C@@H]3CCCN(CCO)C3)n3nccc23)c(C(F)(F)F)c1] | *** |
| I-455 | 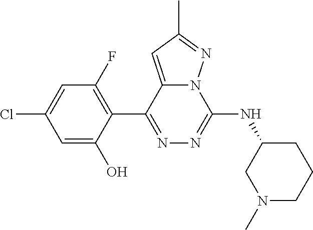 [SMILES: Cc1cc2c(-c3c(O)cc(Cl)cc3F)nnc(N[C@@H]3CCCN(C)C3)n2n1] | ***** |
| I-456 | 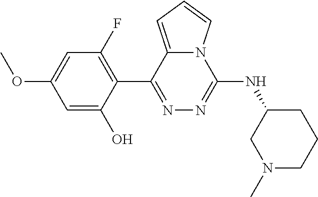 [SMILES: COc1cc(O)c(-c2nnc(N[C@@H]3CCCN(C)C3)n3cccc23)c(F)c1] | ***** |
| I-457 | 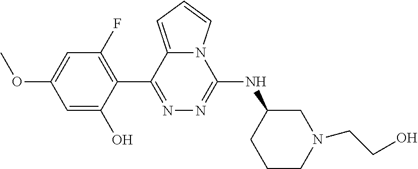 [SMILES: COc1cc(O)c(-c2nnc(N[C@@H]3CCCN(CCO)C3)n3cccc23)c(F)c1] | **** |
| I-458 | 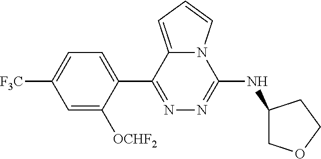 [SMILES: FC(F)Oc1cc(C(F)(F)F)ccc1-c1nnc(N[C@H]2CCOC2)n2cccc12] | ** |
| I-459 | 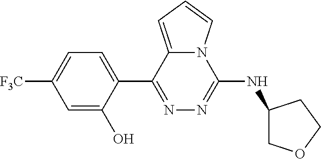 [SMILES: Oc1cc(C(F)(F)F)ccc1-c1nnc(N[C@H]2CCOC2)n2cccc12] | *** |
| I-460 | 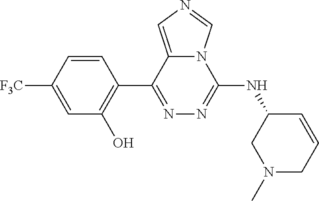 [SMILES: CN1CC=C[C@@H](Nc2nnc(-c3ccc(C(F)(F)F)cc3O)c3cncn23)C1] | **** |
| I-461 | 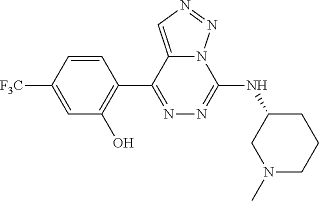 [SMILES: CN1CCC[C@@H](Nc2nnc(-c3ccc(C(F)(F)F)cc3O)c3cnnn23)C1] | **** |
| I-462 | 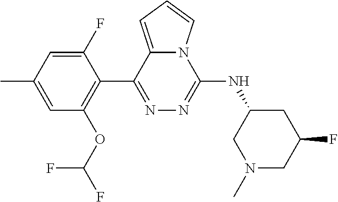 [SMILES: Cc1cc(F)c(-c2nnc(N[C@@H]3C[C@@H](F)CN(C)C3)n3cccc23)c(OC(F)F)c1] | *** |
| I-463 | 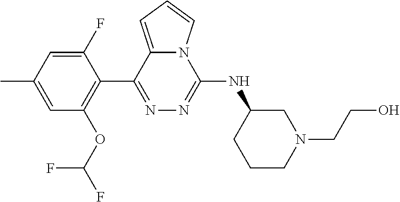 [SMILES: Cc1cc(F)c(-c2nnc(N[C@@H]3CCCN(CCO)C3)n3cccc23)c(OC(F)F)c1] | *** |
| I-464 | 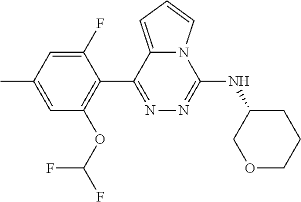 [SMILES: Cc1cc(F)c(-c2nnc(N[C@@H]3CCCOC3)n3cccc23)c(OC(F)F)c1] | ** |
| I-465 | 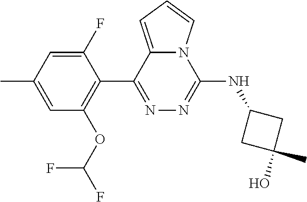 | ** |
| I-466 | 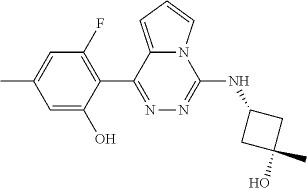 | **** |
| I-467 | 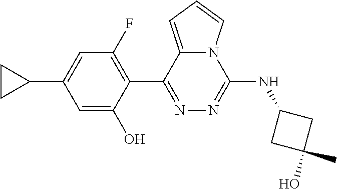 [SMILES: C[C@]1(O)C[C@@H](Nc2nnc(-c3c(O)cc(C4CC4)cc3F)c3cccn23)C1] | **** |
| I-468 | 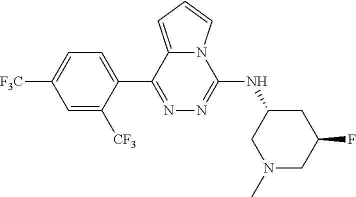 [SMILES: CN1C[C@H](F)C[C@@H](Nc2nnc(-c3ccc(C(F)(F)F)cc3C(F)(F)F)c3cccn23)C1] | **** |
| I-469 | 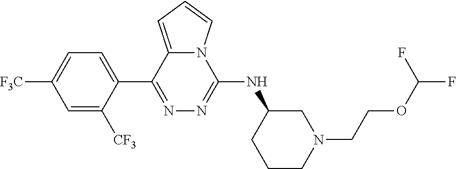 [SMILES: FC(F)OCCN1CCC[C@@H](Nc2nnc(-c3ccc(C(F)(F)F)cc3C(F)(F)F)c3cccn23)C1] | *** |
| I-470 | 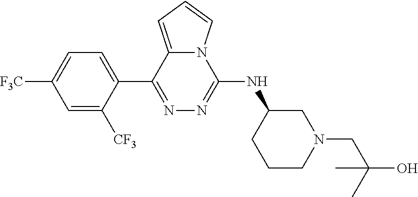 [SMILES: CC(C)(O)CN1CCC[C@@H](Nc2nnc(-c3ccc(C(F)(F)F)cc3C(F)(F)F)c3cccn23)C1] | *** |
| I-471 | 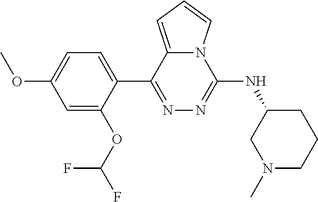 [SMILES: COc1ccc(-c2nnc(N[C@@H]3CCCN(C)C3)n3cccc23)c(OC(F)F)c1] | **** |
| I-472 | 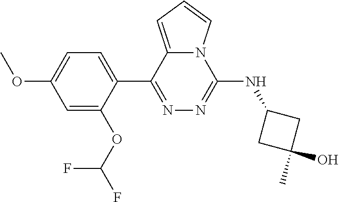 [SMILES: COc1ccc(-c2nnc(N[C@H]3C[C@](C)(O)C3)n3cccc23)c(OC(F)F)c1] | *** |
| I-473 | 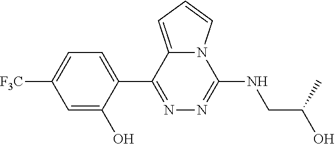 [SMILES: C[C@H](O)CNc1nnc(-c2ccc(C(F)(F)F)cc2O)c2cccn12] | *** |
| I-474 | 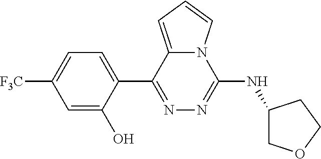 [SMILES: Oc1cc(C(F)(F)F)ccc1-c1nnc(N[C@@H]2CCOC2)n2cccc12] | *** |
| I-475 | 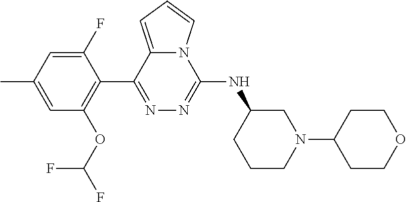 [SMILES: Cc1cc(F)c(-c2nnc(N[C@@H]3CCCN(C4CCOCC4)C3)n3cccc23)c(OC(F)F)c1] | *** |
| I-476 | 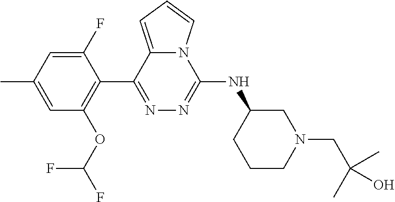 [SMILES: Cc1cc(F)c(-c2nnc(N[C@@H]3CCCN(CC(C)(C)O)C3)n3cccc23)c(OC(F)F)c1] | ** |
| I-477 | 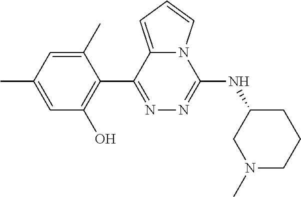 [SMILES: Cc1cc(C)c(-c2nnc(N[C@@H]3CCCN(C)C3)n3cccc23)c(O)c1] | **** |
| I-478 | 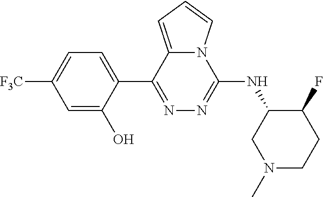 [SMILES: CN1CC[C@H](F)[C@@H](Nc2nnc(-c3ccc(C(F)(F)F)cc3O)c3cccn23)C1] | *** |
| I-479 | 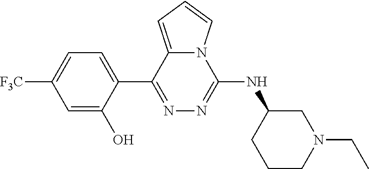 [SMILES: CCN1CCC[C@@H](Nc2nnc(-c3ccc(C(F)(F)F)cc3O)c3cccn23)C1] | ***** |
| I-480 | 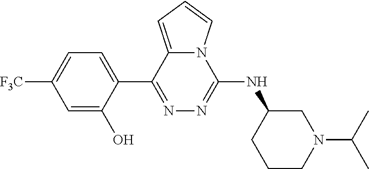 [SMILES: CC(C)N1CCC[C@@H](Nc2nnc(-c3ccc(C(F)(F)F)cc3O)c3cccn23)C1] | **** |
| I-481 | 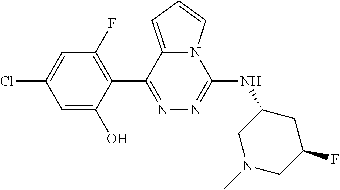 [SMILES: CN1C[C@H](F)C[C@@H](Nc2nnc(-c3c(O)cc(Cl)cc3F)c3cccn23)C1] | **** |
| I-482 | 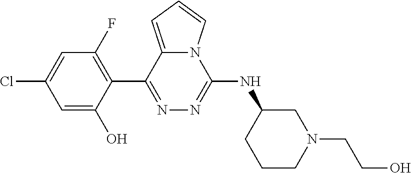 [SMILES: OCCN1CCC[C@@H](Nc2nnc(-c3c(O)cc(Cl)cc3F)c3cccn23)C1] | **** |
| I-483 | 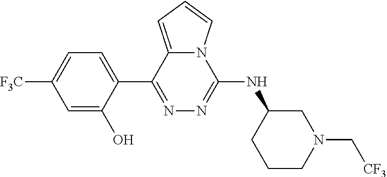 [SMILES: Oc1cc(C(F)(F)F)ccc1-c1nnc(N[C@@H]2CCCN(CC(F)(F)F)C2)n2cccc12] | ** |
| I-484 | 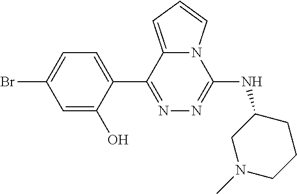 [SMILES: CN1CCC[C@@H](Nc2nnc(-c3ccc(Br)cc3O)c3cccn23)C1] | **** |
| I-485 | 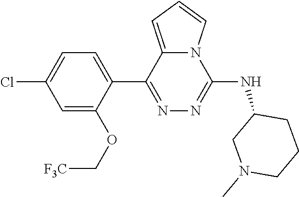 [SMILES: CN1CCC[C@@H](Nc2nnc(-c3ccc(Cl)cc3OCC(F)(F)F)c3cccn23)C1] | *** |
| I-486 | 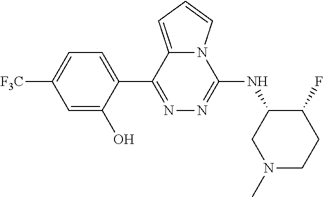 [SMILES: CN1CC[C@@H](F)[C@@H](Nc2nnc(-c3ccc(C(F)(F)F)cc3O)c3cccn23)C1] | *** |
| I-487 | 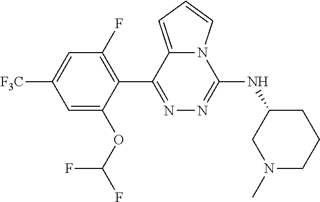 [SMILES: CN1CCC[C@@H](Nc2nnc(-c3c(F)cc(C(F)(F)F)cc3OC(F)F)c3cccn23)C1] | **** |
| I-488 | 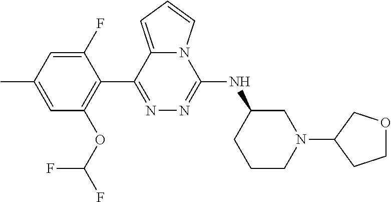 [SMILES: Cc1cc(F)c(-c2nnc(N[C@@H]3CCCN(C4CCOC4)C3)n3cccc23)c(OC(F)F)c1] | *** |
| I-489 | 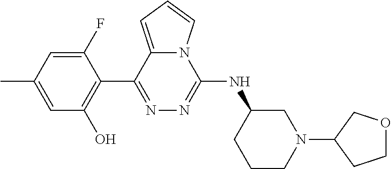 [SMILES: Cc1cc(O)c(-c2nnc(N[C@@H]3CCCN(C4CCOC4)C3)n3cccc23)c(F)c1] | **** |
| I-490 | 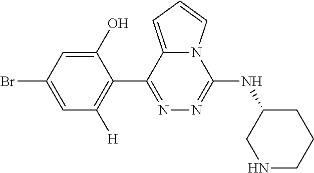 [SMILES: Oc1cc(Br)ccc1-c1nnc(N[C@@H]2CCCNC2)n2cccc12] | **** |
| I-491 | 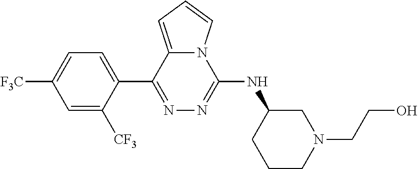 [SMILES: OCCN1CCC[C@@H](Nc2nnc(-c3ccc(C(F)(F)F)cc3C(F)(F)F)c3cccn23)C1] | **** |
| I-492 | 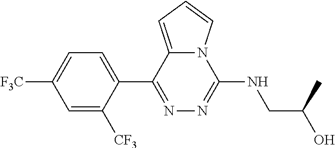 [SMILES: C[C@@H](O)CNc1nnc(-c2ccc(C(F)(F)F)cc2C(F)(F)F)c2cccn12] | **** |
| I-493 | 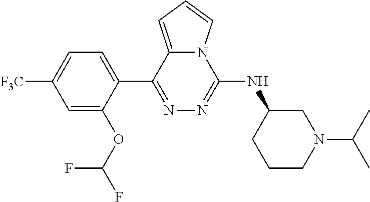 [SMILES: CC(C)N1CCC[C@@H](Nc2nnc(-c3ccc(C(F)(F)F)cc3OC(F)F)c3cccn23)C1] | **** |
| I-494 | 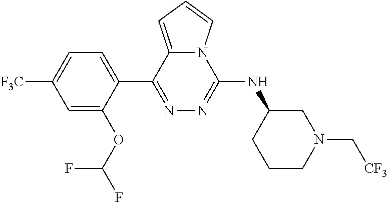 [SMILES: FC(F)Oc1cc(C(F)(F)F)ccc1-c1nnc(N[C@@H]2CCCN(CC(F)(F)F)C2)n2cccc12] | ** |
| I-495 | 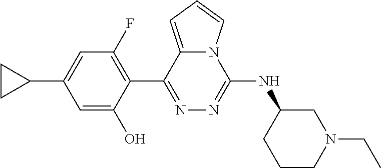 [SMILES: CCN1CCC[C@@H](Nc2nnc(-c3c(O)cc(C4CC4)cc3F)c3cccn23)C1] | ***** |
| I-496 | 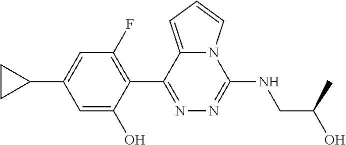 [SMILES: C[C@@H](O)CNc1nnc(-c2c(O)cc(C3CC3)cc2F)c2cccn12] | *** |
| I-497 | 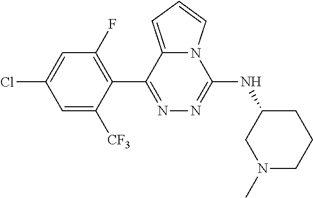 [SMILES: CN1CCC[C@@H](Nc2nnc(-c3c(F)cc(Cl)cc3C(F)(F)F)c3cccn23)C1] | **** |
| I-498 | 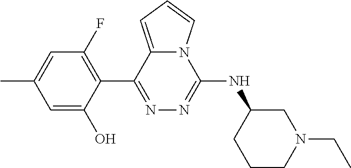 [SMILES: CCN1CCC[C@@H](Nc2nnc(-c3c(O)cc(C)cc3F)c3cccn23)C1] | ***** |
| I-499 | 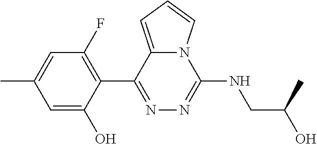 [SMILES: Cc1cc(O)c(-c2nnc(NC[C@@H](C)O)n3cccc23)c(F)c1] | *** |
| I-500 | 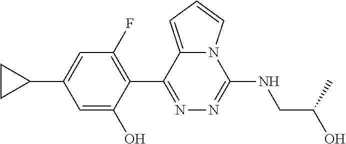 [SMILES: C[C@H](O)CNc1nnc(-c2c(O)cc(C3CC3)cc2F)c2cccn12] | *** |
| I-501 | 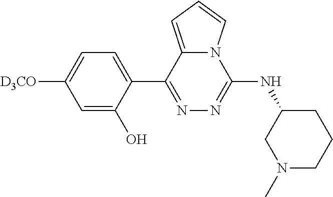 | ***** |
| I-502 | 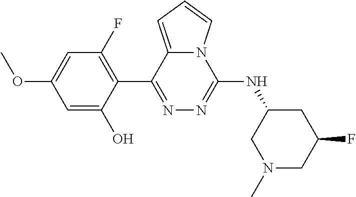 [SMILES: COc1cc(O)c(-c2nnc(N[C@@H]3C[C@@H](F)CN(C)C3)n3cccc23)c(F)c1] | ***** |
| I-504 | 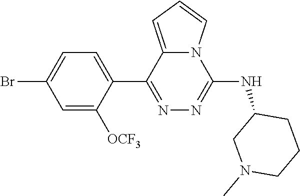 [SMILES: CN1CCC[C@@H](Nc2nnc(-c3ccc(Br)cc3OC(F)(F)F)c3cccn23)C1] | ***** |
| I-505 | 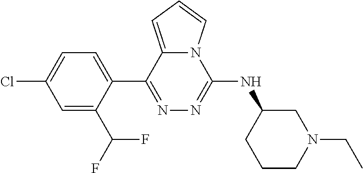 [SMILES: CCN1CCC[C@@H](Nc2nnc(-c3ccc(Cl)cc3C(F)F)c3cccn23)C1] | *** |
| I-506 | 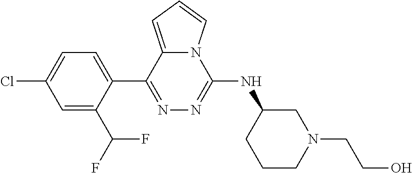 [SMILES: OCCN1CCC[C@@H](Nc2nnc(-c3ccc(Cl)cc3C(F)F)c3cccn23)C1] | *** |
| I-507 | 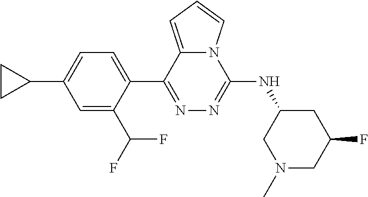 [SMILES: CN1C[C@H](F)C[C@@H](Nc2nnc(-c3ccc(C4CC4)cc3C(F)F)c3cccn23)C1] | *** |
| I-508 | 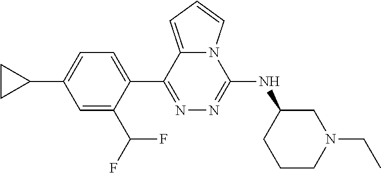 [SMILES: CCN1CCC[C@@H](Nc2nnc(-c3ccc(C4CC4)cc3C(F)F)c3cccn23)C1] | **** |
| I-509 | 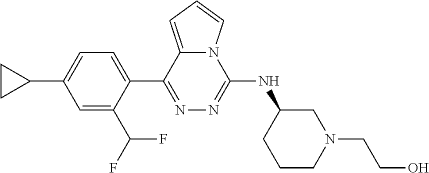 [SMILES: OCCN1CCC[C@@H](Nc2nnc(-c3ccc(C4CC4)cc3C(F)F)c3cccn23)C1] | **** |
| I-510 | 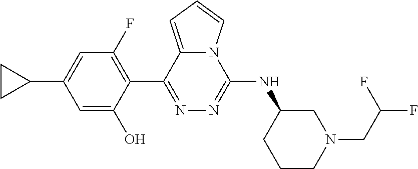 [SMILES: Oc1cc(C2CC2)cc(F)c1-c1nnc(N[C@@H]2CCCN(CC(F)F)C2)n2cccc12] | *** |
| I-511 | 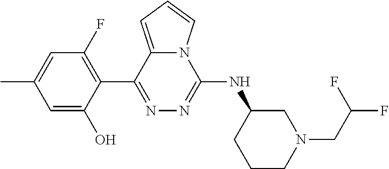 [SMILES: Cc1cc(O)c(-c2nnc(N[C@@H]3CCCN(CC(F)F)C3)n3cccc23)c(F)c1] | **** |
| I-512 | 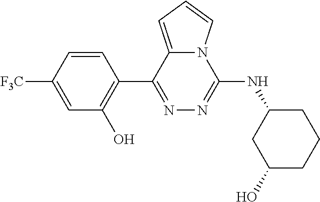 [SMILES: Oc1cc(C(F)(F)F)ccc1-c1nnc(N[C@@H]2CCC[C@H](O)C2)n2cccc12] | **** |
| I-513 | 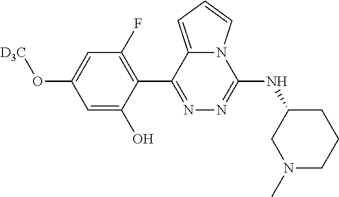 [SMILES: [2H]C([2H])([2H])Oc1cc(O)c(-c2nnc(N[C@@H]3CCCN(C)C3)n3cccc23)c(F)c1] | ***** |
| I-514 | 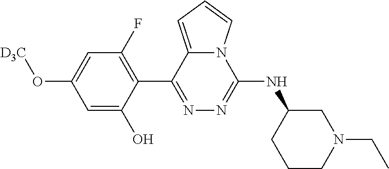 [SMILES: [2H]C([2H])([2H])Oc1cc(O)c(-c2nnc(N[C@@H]3CCCN(CC)C3)n3cccc23)c(F)c1] | ***** |
| I-515 | 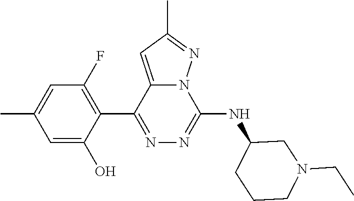 [SMILES: CCN1CCC[C@@H](Nc2nnc(-c3c(O)cc(C)cc3F)c3cc(C)nn23)C1] | ***** |
| I-516 | 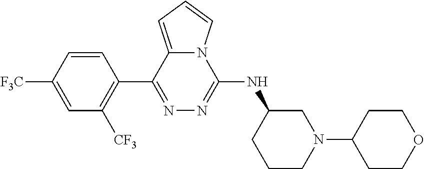 [SMILES: FC(F)(F)c1ccc(-c2nnc(N[C@@H]3CCCN(C4CCOCC4)C3)n3cccc23)c(C(F)(F)F)c1] | **** |
| I-517 | 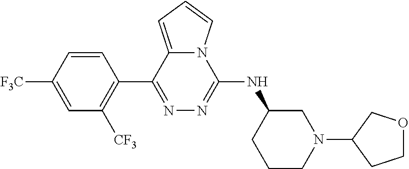 [SMILES: FC(F)(F)c1ccc(-c2nnc(N[C@@H]3CCCN(C4CCOC4)C3)n3cccc23)c(C(F)(F)F)c1] | **** |
| I-518 | 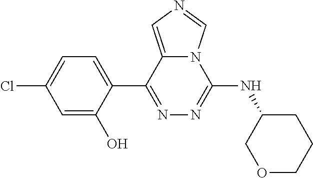 [SMILES: Oc1cc(Cl)ccc1-c1nnc(N[C@@H]2CCCOC2)n2cncc12] | **** |
| I-519 | 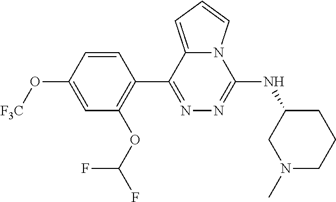 [SMILES: CN1CCC[C@@H](Nc2nnc(-c3ccc(OC(F)(F)F)cc3OC(F)F)c3cccn23)C1] | **** |
| I-520 | 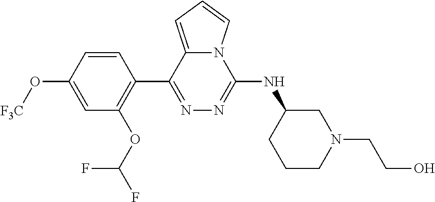 [SMILES: OCCN1CCC[C@@H](Nc2nnc(-c3ccc(OC(F)(F)F)cc3OC(F)F)c3cccn23)C1] | **** |
| I-521 | 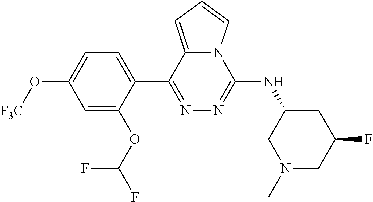 [SMILES: CN1C[C@H](F)C[C@@H](Nc2nnc(-c3ccc(OC(F)(F)F)cc3OC(F)F)c3cccn23)C1] | **** |
| I-522 | 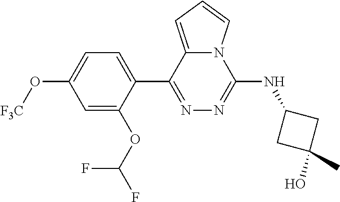 [SMILES: C[C@]1(O)C[C@@H](Nc2nnc(-c3ccc(OC(F)(F)F)cc3OC(F)F)c3cccn23)C1] | *** |
| I-523 | 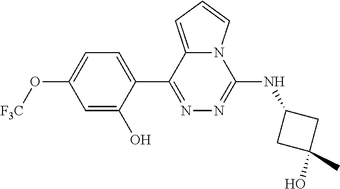 [SMILES: C[C@]1(O)C[C@@H](Nc2nnc(-c3ccc(OC(F)(F)F)cc3O)c3cccn23)C1] | *** |
| I-524 | 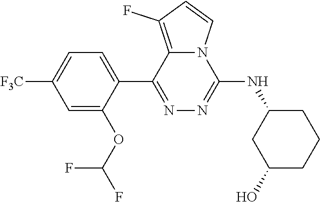 [SMILES: O[C@H]1CCC[C@@H](Nc2nnc(-c3ccc(C(F)(F)F)cc3OC(F)F)c3c(F)ccn23)C1] | *** |
| I-525 | 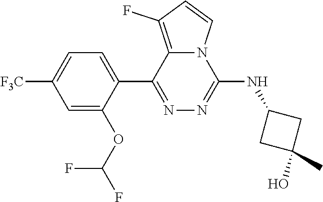 [SMILES: C[C@]1(O)C[C@@H](Nc2nnc(-c3ccc(C(F)(F)F)cc3OC(F)F)c3c(F)ccn23)C1] | *** |
| I-526 | 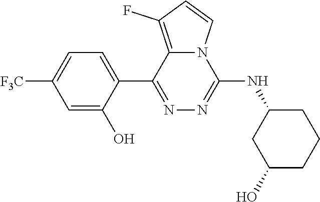 [SMILES: Oc1cc(C(F)(F)F)ccc1-c1nnc(N[C@@H]2CCC[C@H](O)C2)n2ccc(F)c12] | *** |
| I-527 | 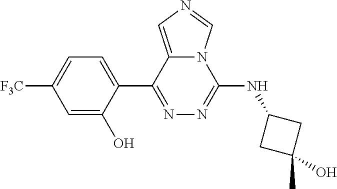 [SMILES: C[C@]1(O)C[C@@H](Nc2nnc(-c3ccc(C(F)(F)F)cc3O)c3cncn23)C1] | **** |
| I-528 | 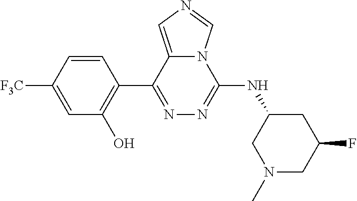 [SMILES: CN1C[C@H](F)C[C@@H](Nc2nnc(-c3ccc(C(F)(F)F)cc3O)c3cncn23)C1] | ***** |
| I-529 | 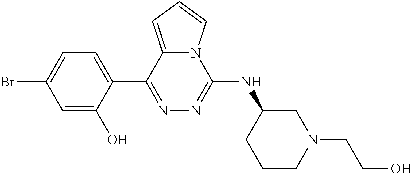 [SMILES: OCCN1CCC[C@@H](Nc2nnc(-c3ccc(Br)cc3O)c3cccn23)C1] | **** |
| I-530 | 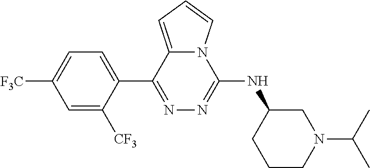 [SMILES: CC(C)N1CCC[C@@H](Nc2nnc(-c3ccc(C(F)(F)F)cc3C(F)(F)F)c3cccn23)C1] | ***** |
| I-531 | 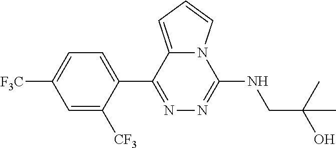 [SMILES: CC(C)(O)CNc1nnc(-c2ccc(C(F)(F)F)cc2C(F)(F)F)c2cccn12] | *** |
| I-532 | 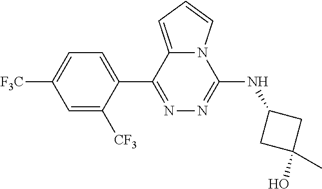 [SMILES: C[C@]1(O)C[C@@H](Nc2nnc(-c3ccc(C(F)(F)F)cc3C(F)(F)F)c3cccn23)C1] | **** |
| I-533 | 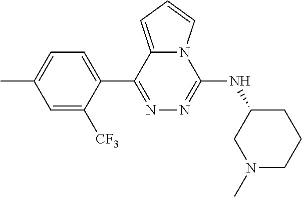 [SMILES: Cc1ccc(-c2nnc(N[C@@H]3CCCN(C)C3)n3cccc23)c(C(F)(F)F)c1] | **** |
| I-534 | 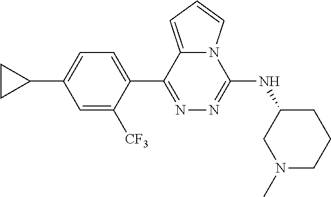 [SMILES: CN1CCC[C@@H](Nc2nnc(-c3ccc(C4CC4)cc3C(F)(F)F)c3cccn23)C1] | ***** |
| I-535 | 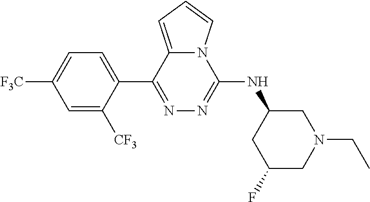 [SMILES: CCN1C[C@H](F)C[C@@H](Nc2nnc(-c3ccc(C(F)(F)F)cc3C(F)(F)F)c3cccn23)C1] | **** |
| I-536 | 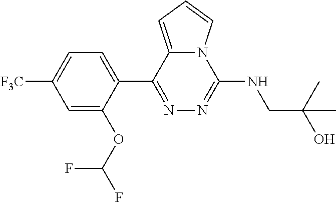 [SMILES: CC(C)(O)CNc1nnc(-c2ccc(C(F)(F)F)cc2OC(F)F)c2cccn12] | *** |
| I-537 | 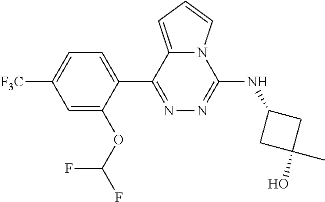 [SMILES: C[C@]1(O)C[C@@H](Nc2nnc(-c3ccc(C(F)(F)F)cc3OC(F)F)c3cccn23)C1] | **** |
| I-538 | 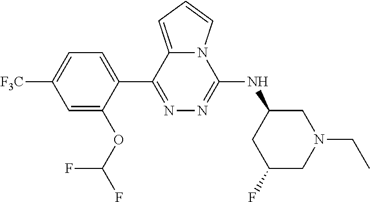 [SMILES: CCN1C[C@H](F)C[C@@H](Nc2nnc(-c3ccc(C(F)(F)F)cc3OC(F)F)c3cccn23)C1] | **** |
| I-539 | 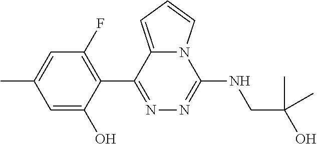 [SMILES: Cc1cc(O)c(-c2nnc(NCC(C)(C)O)n3cccc23)c(F)c1] | **** |
| I-540 | 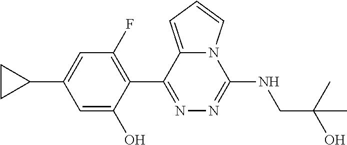 [SMILES: CC(C)(O)CNc1nnc(-c2c(O)cc(C3CC3)cc2F)c2cccn12] | *** |
| I-541 | 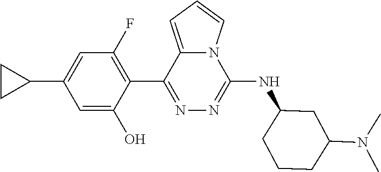 [SMILES: CN(C)C1CCC[C@@H](Nc2nnc(-c3c(O)cc(C4CC4)cc3F)c3cccn23)C1] | ***** |
| I-542 | 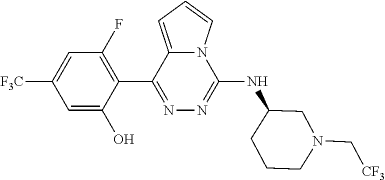 [SMILES: Oc1cc(C(F)(F)F)cc(F)c1-c1nnc(N[C@@H]2CCCN(CC(F)(F)F)C2)n2cccc12] | *** |
| I-543 | 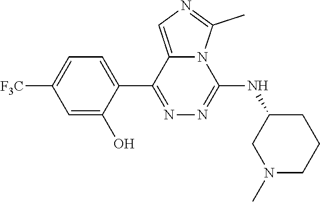 [SMILES: Cc1ncc2c(-c3ccc(C(F)(F)F)cc3O)nnc(N[C@@H]3CCCN(C)C3)n12] | ***** |
| I-544 | 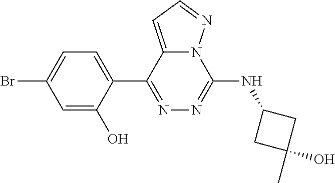 [SMILES: C[C@]1(O)C[C@@H](Nc2nnc(-c3ccc(Br)cc3O)c3ccnn23)C1] | **** |
| I-545 | 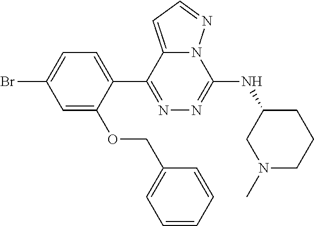 [SMILES: CN1CCC[C@@H](Nc2nnc(-c3ccc(Br)cc3OCc3ccccc3)c3ccnn23)C1] | *** |
| I-546 | 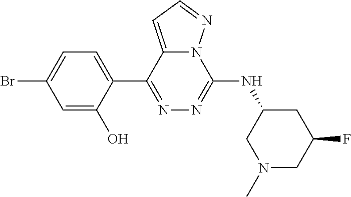 [SMILES: CN1C[C@H](F)C[C@@H](Nc2nnc(-c3ccc(Br)cc3O)c3ccnn23)C1] | **** |
| I-547 | 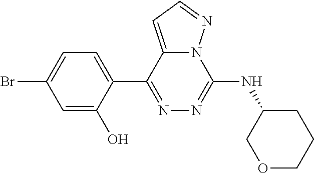 [SMILES: Oc1cc(Br)ccc1-c1nnc(N[C@@H]2CCCOC2)n2nccc12] | *** |
| I-548 | 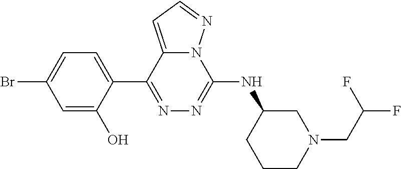 [SMILES: Oc1cc(Br)ccc1-c1nnc(N[C@@H]2CCCN(CC(F)F)C2)n2nccc12] | **** |
| I-549 | 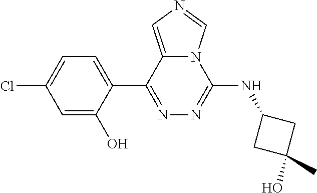 [SMILES: C[C@]1(O)C[C@@H](Nc2nnc(-c3ccc(Cl)cc3O)c3cncn23)C1] | **** |
| I-550 | 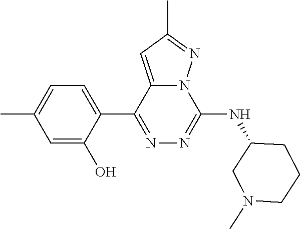 [SMILES: Cc1ccc(-c2nnc(N[C@@H]3CCCN(C)C3)n3nc(C)cc23)c(O)c1] | **** |
| I-551 | 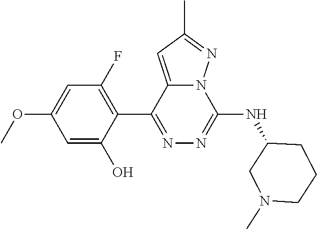 [SMILES: COc1cc(O)c(-c2nnc(N[C@@H]3CCCN(C)C3)n3nc(C)cc23)c(F)c1] | ***** |
| I-552 | 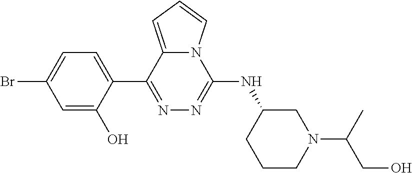 [SMILES: CC(CO)N1CCC[C@H](Nc2nnc(-c3ccc(Br)cc3O)c3cccn23)C1] | **** |
| I-553 | 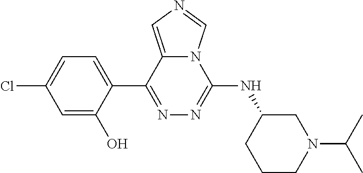 [SMILES: CC(C)N1CCC[C@H](Nc2nnc(-c3ccc(Cl)cc3O)c3cncn23)C1] | ***** |
| I-554 | 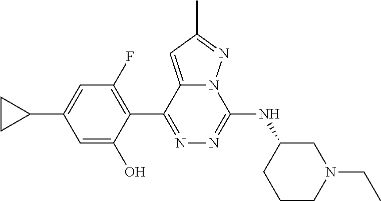 [SMILES: CCN1CCC[C@H](Nc2nnc(-c3c(O)cc(C4CC4)cc3F)c3cc(C)nn23)C1] | ***** |
| I-555 | 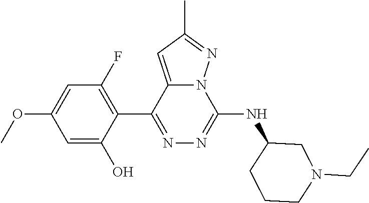 [SMILES: CCN1CCC[C@@H](Nc2nnc(-c3c(O)cc(OC)cc3F)c3cc(C)nn23)C1] | ***** |
| I-556 | 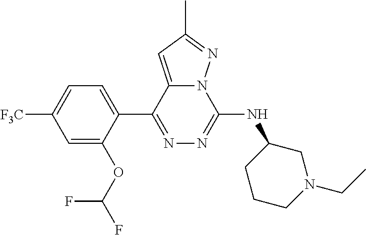 [SMILES: CCN1CCC[C@@H](Nc2nnc(-c3ccc(C(F)(F)F)cc3OC(F)F)c3cc(C)nn23)C1] | **** |
| I-557 | 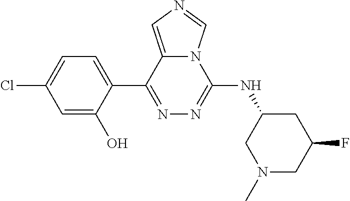 [SMILES: CN1C[C@H](F)C[C@@H](Nc2nnc(-c3ccc(Cl)cc3O)c3cncn23)C1] | **** |
| I-558 | 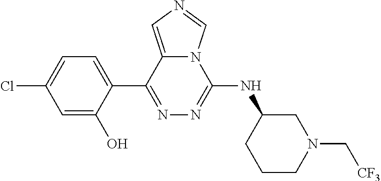 [SMILES: Oc1cc(Cl)ccc1-c1nnc(N[C@@H]2CCCN(CC(F)(F)F)C2)n2cncc12] | **** |
| I-559 | 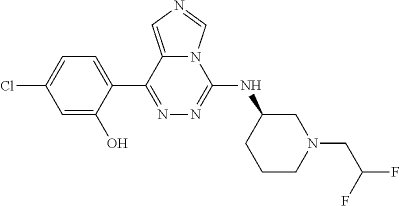 [SMILES: Oc1cc(Cl)ccc1-c1nnc(N[C@@H]2CCCN(CC(F)F)C2)n2cncc12] | **** |
| I-560 | 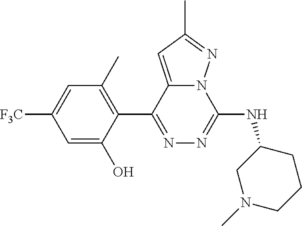 [SMILES: Cc1cc2c(-c3c(C)cc(C(F)(F)F)cc3O)nnc(N[C@@H]3CCCN(C)C3)n2n1] | **** |
| I-561 | 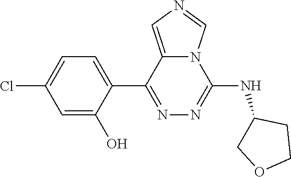 [SMILES: Oc1cc(Cl)ccc1-c1nnc(N[C@@H]2CCOC2)n2cncc12] | **** |
| I-562 | 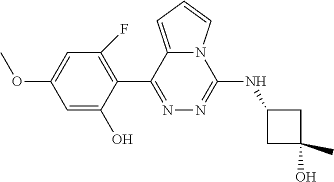 | **** |
| I-563 | 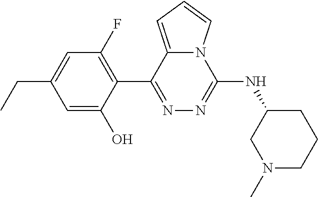 [SMILES: CCc1cc(O)c(-c2nnc(N[C@@H]3CCCN(C)C3)n3cccc23)c(F)c1] | ***** |
| I-564 | 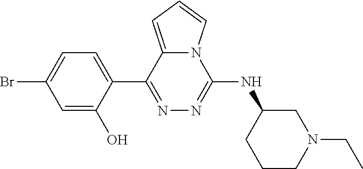 [SMILES: CCN1CCC[C@@H](Nc2nnc(-c3ccc(Br)cc3O)c3cccn23)C1] | ***** |
| I-565 | 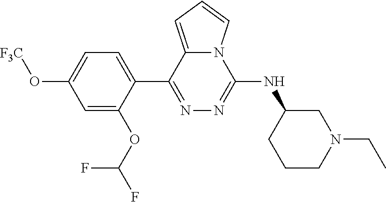 [SMILES: CCN1CCC[C@@H](Nc2nnc(-c3ccc(OC(F)(F)F)cc3OC(F)F)c3cccn23)C1] | **** |
| I-566 | 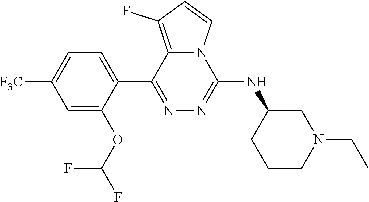 [SMILES: CCN1CCC[C@@H](Nc2nnc(-c3ccc(C(F)(F)F)cc3OC(F)F)c3c(F)ccn23)C1] | **** |
| I-567 | 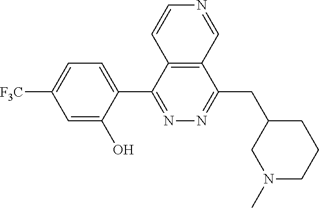 [SMILES: CN1CCCC(Cc2nnc(-c3ccc(C(F)(F)F)cc3O)c3ccncc23)C1] | *** |
| I-568 | 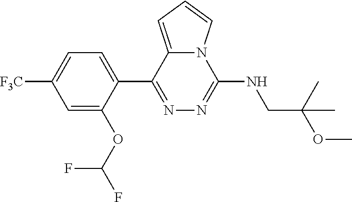 [SMILES: COC(C)(C)CNc1nnc(-c2ccc(C(F)(F)F)cc2OC(F)F)c2cccn12] | **** |
| I-569 | 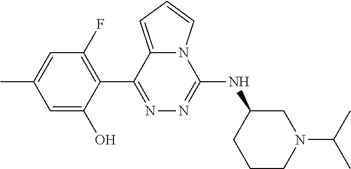 [SMILES: Cc1cc(O)c(-c2nnc(N[C@@H]3CCCN(C(C)C)C3)n3cccc23)c(F)c1] | ***** |
| I-570 | 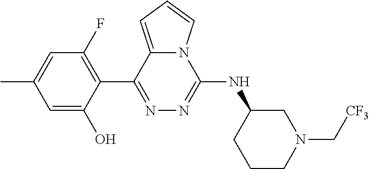 [SMILES: Cc1cc(O)c(-c2nnc(N[C@@H]3CCCN(CC(F)(F)F)C3)n3cccc23)c(F)c1] | **** |
| I-571 | 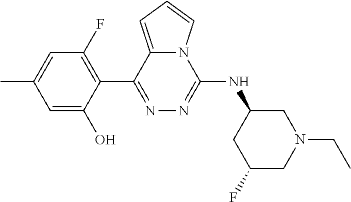 [SMILES: CCN1C[C@H](F)C[C@@H](Nc2nnc(-c3c(O)cc(C)cc3F)c3cccn23)C1] | ***** |
| I-572 | 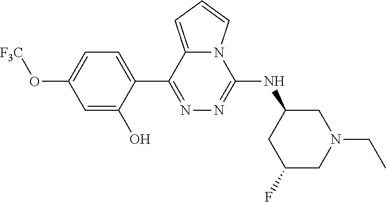 [SMILES: CCN1C[C@H](F)C[C@@H](Nc2nnc(-c3ccc(OC(F)(F)F)cc3O)c3cccn23)C1] | **** |
| I-573 | 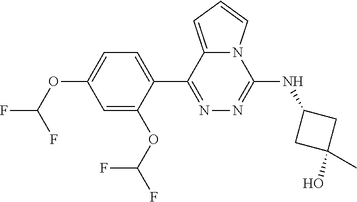 [SMILES: C[C@]1(O)C[C@@H](Nc2nnc(-c3ccc(OC(F)F)cc3OC(F)F)c3cccn23)C1] | *** |
| I-574 | 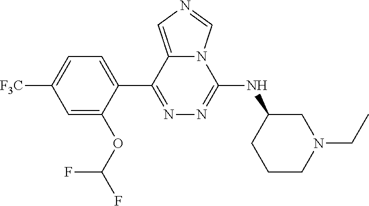 [SMILES: CCN1CCC[C@@H](Nc2nnc(-c3ccc(C(F)(F)F)cc3OC(F)F)c3cncn23)C1] | ***** |
| I-575 | 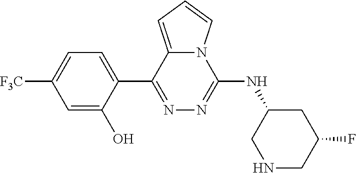 [SMILES: Oc1cc(C(F)(F)F)ccc1-c1nnc(N[C@H]2CNC[C@@H](F)C2)n2cccc12] | ** |
| I-576 | 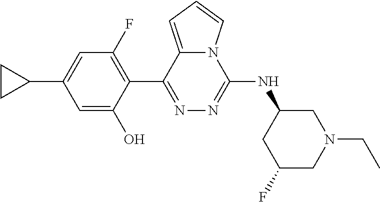 [SMILES: CCN1C[C@H](F)C[C@@H](Nc2nnc(-c3c(O)cc(C4CC4)cc3F)c3cccn23)C1] | ***** |
| I-577 | 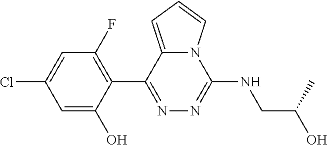 [SMILES: C[C@H](O)CNc1nnc(-c2c(O)cc(Cl)cc2F)c2cccn12] | *** |
| I-578 | 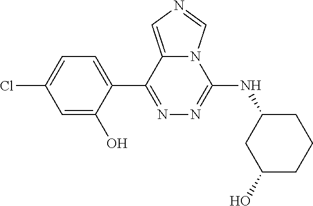 [SMILES: Oc1cc(Cl)ccc1-c1nnc(N[C@@H]2CCC[C@H](O)C2)n2cncc12] | ***** |
| I-579 | 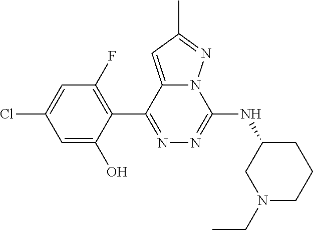 [SMILES: CCN1CCC[C@@H](Nc2nnc(-c3c(O)cc(Cl)cc3F)c3cc(C)nn23)C1] | ***** |
| I-580 | 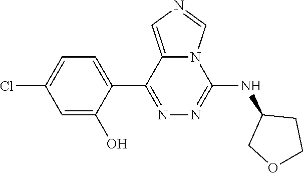 [SMILES: Oc1cc(Cl)ccc1-c1nnc(N[C@H]2CCOC2)n2cncc12] | **** |
| I-581 | 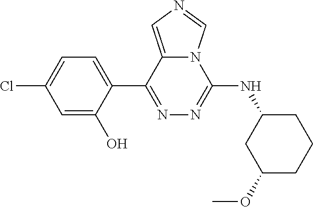 [SMILES: CO[C@H]1CCC[C@@H](Nc2nnc(-c3ccc(Cl)cc3O)c3cncn23)C1] | **** |
| I-582 | 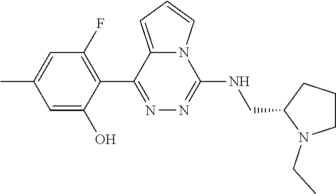 [SMILES: CCN1CCC[C@H]1CNc1nnc(-c2c(O)cc(C)cc2F)c2cccn12] | **** |
| I-583 | 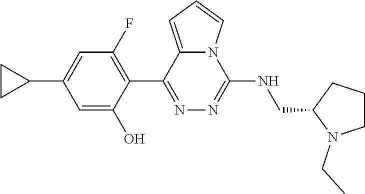 [SMILES: CCN1CCC[C@H]1CNc1nnc(-c2c(O)cc(C3CC3)cc2F)c2cccn12] | **** |
| I-584 | 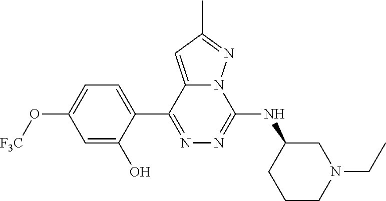 [SMILES: CCN1CCC[C@@H](Nc2nnc(-c3ccc(OC(F)(F)F)cc3O)c3cc(C)nn23)C1] | ***** |
| I-585 | 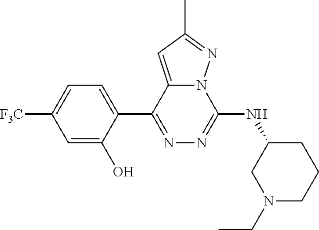 [SMILES: CCN1CCC[C@@H](Nc2nnc(-c3ccc(C(F)(F)F)cc3O)c3cc(C)nn23)C1] | **** |
| I-586 | 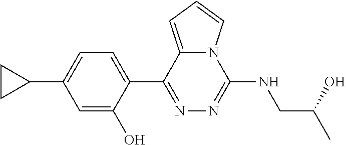 [SMILES: C[C@@H](O)CNc1nnc(-c2ccc(C3CC3)cc2O)c2cccn12] | ** |
| I-587 | 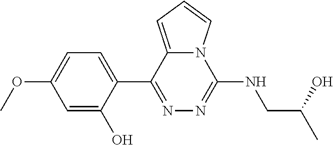 [SMILES: COc1ccc(-c2nnc(NC[C@@H](C)O)n3cccc23)c(O)c1] | *** |
| I-588 | 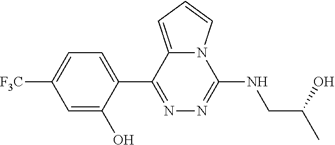 [SMILES: C[C@@H](O)CNc1nnc(-c2ccc(C(F)(F)F)cc2O)c2cccn12] | *** |
| I-589 | 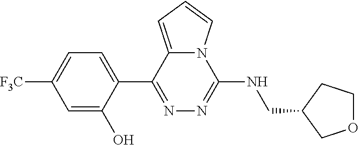 [SMILES: Oc1cc(C(F)(F)F)ccc1-c1nnc(NC[C@@H]2CCOC2)n2cccc12] | *** |
| I-590 | 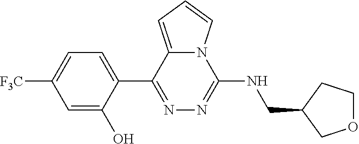 [SMILES: Oc1cc(C(F)(F)F)ccc1-c1nnc(NC[C@H]2CCOC2)n2cccc12] | *** |
| I-591 | 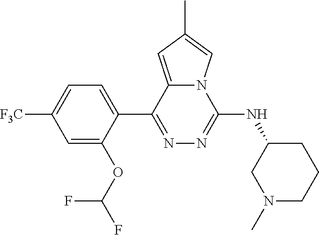 [SMILES: Cc1cc2c(-c3ccc(C(F)(F)F)cc3OC(F)F)nnc(N[C@@H]3CCCN(C)C3)n2c1] | **** |
| I-592 | 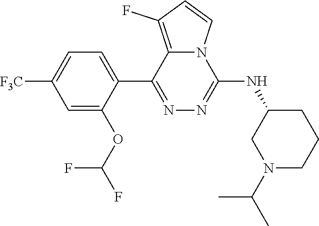 [SMILES: CC(C)N1CCC[C@@H](Nc2nnc(-c3ccc(C(F)(F)F)cc3OC(F)F)c3c(F)ccn23)C1] | ***** |
| I-593 | 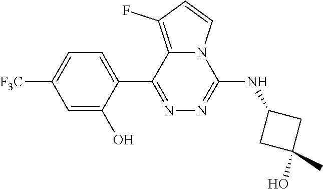 [SMILES: C[C@]1(O)C[C@@H](Nc2nnc(-c3ccc(C(F)(F)F)cc3O)c3c(F)ccn23)C1] | **** |
| I-594 | 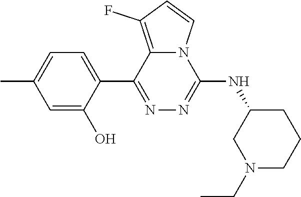 [SMILES: CCN1CCC[C@@H](Nc2nnc(-c3ccc(C)cc3O)c3c(F)ccn23)C1] | ***** |
| I-595 | 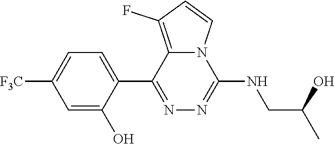 [SMILES: C[C@H](O)CNc1nnc(-c2ccc(C(F)(F)F)cc2O)c2c(F)ccn12] | *** |
| I-596 | 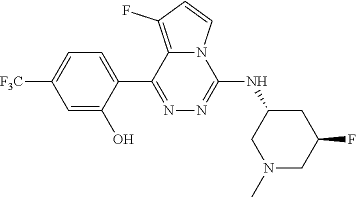 [SMILES: CN1C[C@H](F)C[C@@H](Nc2nnc(-c3ccc(C(F)(F)F)cc3O)c3c(F)ccn23)C1] | **** |
| I-597 | 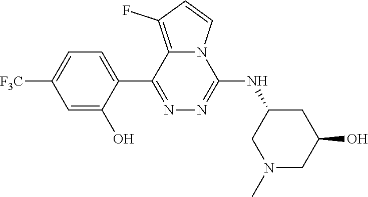 [SMILES: CN1C[C@H](O)C[C@@H](Nc2nnc(-c3ccc(C(F)(F)F)cc3O)c3c(F)ccn23)C1] | *** |
| I-598 | 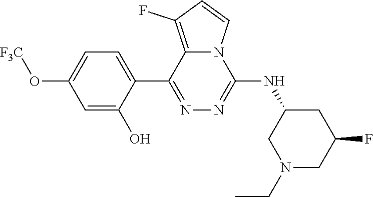 [SMILES: CCN1C[C@H](F)C[C@@H](Nc2nnc(-c3ccc(OC(F)(F)F)cc3O)c3c(F)ccn23)C1] | **** |
| I-599 | 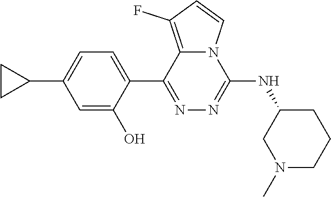 [SMILES: CN1CCC[C@@H](Nc2nnc(-c3ccc(C4CC4)cc3O)c3c(F)ccn23)C1] | **** |
| I-600 | 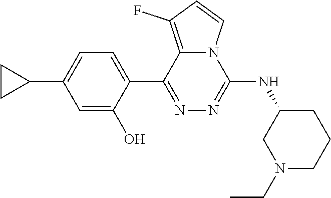 [SMILES: CCN1CCC[C@@H](Nc2nnc(-c3ccc(C4CC4)cc3O)c3c(F)ccn23)C1] | ***** |
| I-601 | 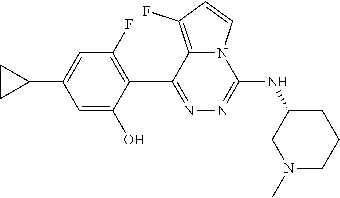 [SMILES: CN1CCC[C@@H](Nc2nnc(-c3c(O)cc(C4CC4)cc3F)c3c(F)ccn23)C1] | ***** |
| I-602 | 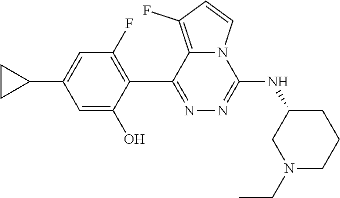 [SMILES: CCN1CCC[C@@H](Nc2nnc(-c3c(O)cc(C4CC4)cc3F)c3c(F)ccn23)C1] | ***** |
| I-603 | 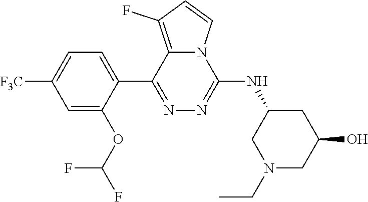 [SMILES: CCN1C[C@H](O)C[C@@H](Nc2nnc(-c3ccc(C(F)(F)F)cc3OC(F)F)c3c(F)ccn23)C1] | ** |
| I-604 | 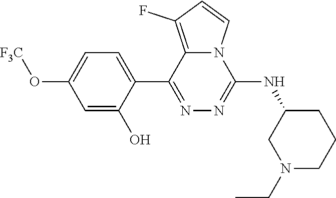 [SMILES: CCN1CCC[C@@H](Nc2nnc(-c3ccc(OC(F)(F)F)cc3O)c3c(F)ccn23)C1] | **** |
| I-605 | 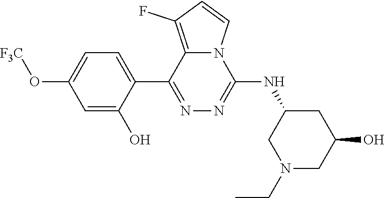 [SMILES: CCN1C[C@H](O)C[C@@H](Nc2nnc(-c3ccc(OC(F)(F)F)cc3O)c3c(F)ccn23)C1] | ** |
| I-606 | 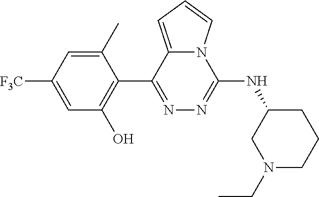 [SMILES: CCN1CCC[C@@H](Nc2nnc(-c3c(C)cc(C(F)(F)F)cc3O)c3cccn23)C1] | ***** |
| I-607 | 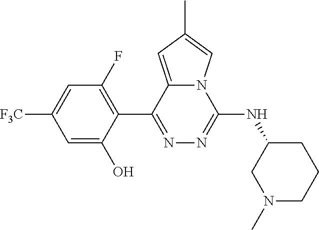 [SMILES: Cc1cc2c(-c3c(O)cc(C(F)(F)F)cc3F)nnc(N[C@@H]3CCCN(C)C3)n2c1] | ***** |
| I-608 | 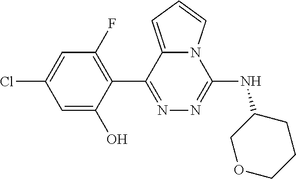 | *** |
| I-609 | 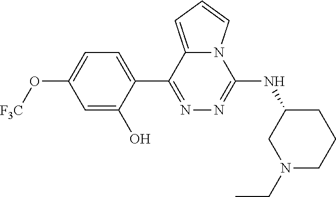 [SMILES: CCN1CCC[C@@H](Nc2nnc(-c3ccc(OC(F)(F)F)cc3O)c3cccn23)C1] | **** |
| I-610 | 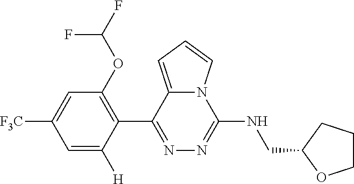 [SMILES: FC(F)Oc1cc(C(F)(F)F)ccc1-c1nnc(NC[C@@H]2CCCO2)n2cccc12] | *** |
| I-611 | 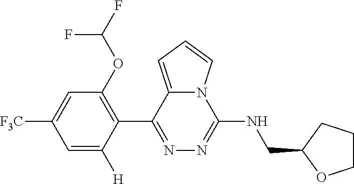 [SMILES: FC(F)Oc1cc(C(F)(F)F)ccc1-c1nnc(NC[C@H]2CCCO2)n2cccc12] | *** |
| I-612 | 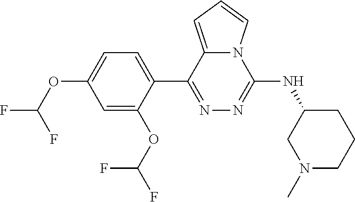 [SMILES: CN1CCC[C@@H](Nc2nnc(-c3ccc(OC(F)F)cc3OC(F)F)c3cccn23)C1] | **** |
| I-613 | 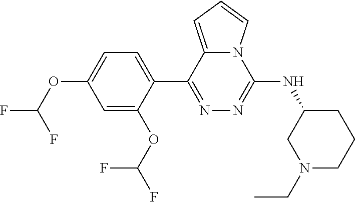 [SMILES: CCN1CCC[C@@H](Nc2nnc(-c3ccc(OC(F)F)cc3OC(F)F)c3cccn23)C1] | **** |
| I-614 | 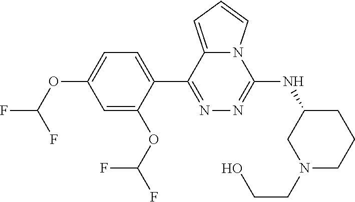 [SMILES: OCCN1CCC[C@@H](Nc2nnc(-c3ccc(OC(F)F)cc3OC(F)F)c3cccn23)C1] | **** |
| I-615 | 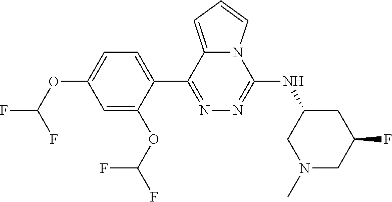 [SMILES: CN1C[C@H](F)C[C@@H](Nc2nnc(-c3ccc(OC(F)F)cc3OC(F)F)c3cccn23)C1] | *** |
| I-616 | 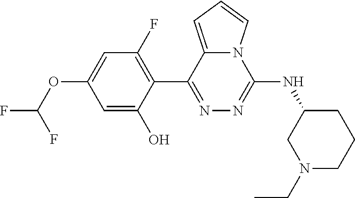 [SMILES: CCN1CCC[C@@H](Nc2nnc(-c3c(O)cc(OC(F)F)cc3F)c3cccn23)C1] | ***** |
| I-617 | 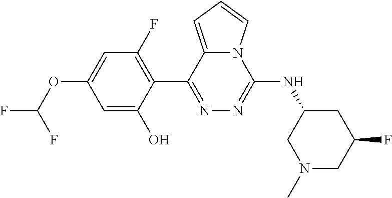 [SMILES: CN1C[C@H](F)C[C@@H](Nc2nnc(-c3c(O)cc(OC(F)F)cc3F)c3cccn23)C1] | **** |
| I-618 | 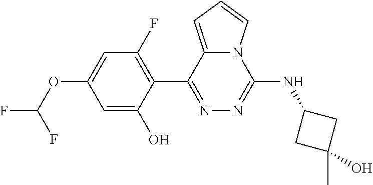 [SMILES: C[C@]1(O)C[C@@H](Nc2nnc(-c3c(O)cc(OC(F)F)cc3F)c3cccn23)C1] | *** |
| I-619 | 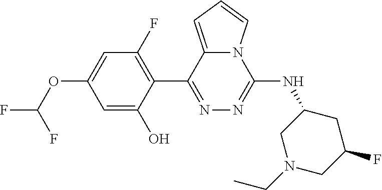 [SMILES: CCN1C[C@H](F)C[C@@H](Nc2nnc(-c3c(O)cc(OC(F)F)cc3F)c3cccn23)C1] | **** |
| I-620 | 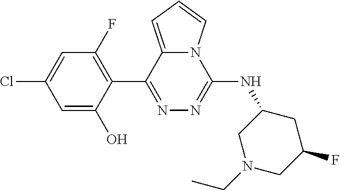 [SMILES: CCN1C[C@H](F)C[C@@H](Nc2nnc(-c3c(O)cc(Cl)cc3F)c3cccn23)C1] | **** |
| I-621 | 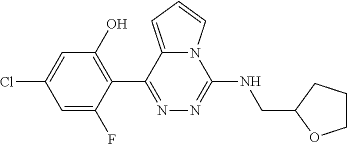 [SMILES: Oc1cc(Cl)cc(F)c1-c1nnc(NCC2CCCO2)n2cccc12] | *** |
| I-623 | 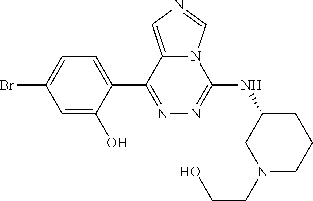 [SMILES: OCCN1CCC[C@@H](Nc2nnc(-c3ccc(Br)cc3O)c3cncn23)C1] | ***** |
| I-624 | 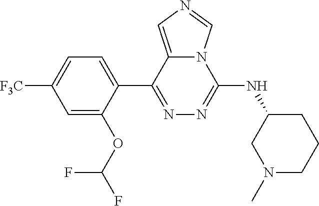 [SMILES: CN1CCC[C@@H](Nc2nnc(-c3ccc(C(F)(F)F)cc3OC(F)F)c3cncn23)C1] | **** |
| I-625 | 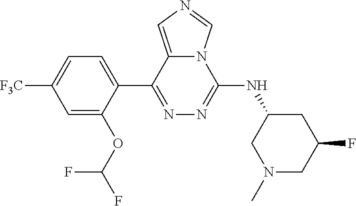 [SMILES: CN1C[C@H](F)C[C@@H](Nc2nnc(-c3ccc(C(F)(F)F)cc3OC(F)F)c3cncn23)C1] | *** |
| I-626 | 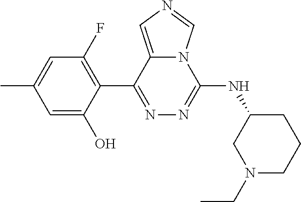 [SMILES: CCN1CCC[C@@H](Nc2nnc(-c3c(O)cc(C)cc3F)c3cncn23)C1] | ***** |
| I-627 | 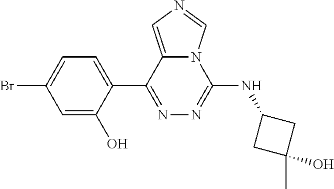 [SMILES: C[C@]1(O)C[C@@H](Nc2nnc(-c3ccc(Br)cc3O)c3cncn23)C1] | **** |
| I-628 | 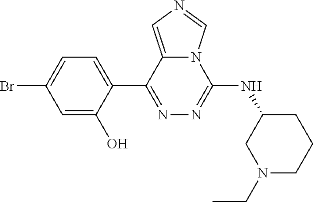 [SMILES: CCN1CCC[C@@H](Nc2nnc(-c3ccc(Br)cc3O)c3cncn23)C1] | ***** |
| I-629 | 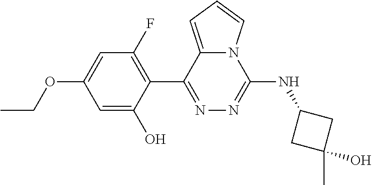 [SMILES: CCOc1cc(O)c(-c2nnc(N[C@H]3C[C@@](C)(O)C3)n3cccc23)c(F)c1] | *** |
| I-630 | 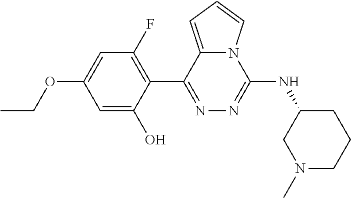 [SMILES: CCOc1cc(O)c(-c2nnc(N[C@@H]3CCCN(C)C3)n3cccc23)c(F)c1] | **** |
| I-631 | 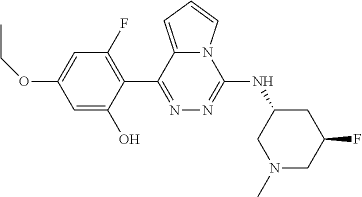 [SMILES: CCOc1cc(O)c(-c2nnc(N[C@@H]3C[C@@H](F)CN(C)C3)n3cccc23)c(F)c1] | *** |
| I-632 | 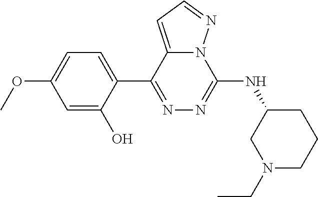 [SMILES: CCN1CCC[C@@H](Nc2nnc(-c3ccc(OC)cc3O)c3ccnn23)C1] | **** |
| I-633 | 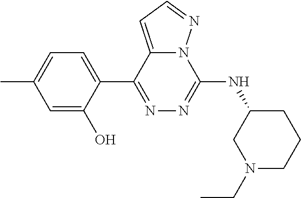 [SMILES: CCN1CCC[C@@H](Nc2nnc(-c3ccc(C)cc3O)c3ccnn23)C1] | **** |
| I-634 | 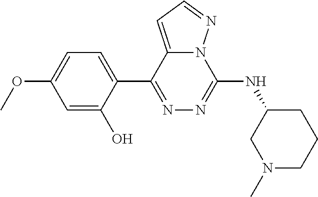 [SMILES: COc1ccc(-c2nnc(N[C@@H]3CCCN(C)C3)n3nccc23)c(O)c1] | *** |
| I-636 | 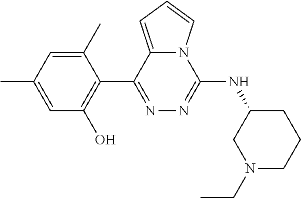 [SMILES: CCN1CCC[C@@H](Nc2nnc(-c3c(C)cc(C)cc3O)c3cccn23)C1] | ***** |

Without regard to whether a document cited herein was specifically and individually indicated as being incorporated by reference, all documents referred to herein are incorporated by reference into the present application for any and all purposes to the same extent as if each individual reference was fully set forth herein.

Although certain embodiments have been described in detail above, those having ordinary skill in the art will clearly understand that many modifications are possible in the embodiments without departing from the teachings thereof. All such modifications are intended to be encompassed within the scope of the claims presented herein.

What is claimed is:

1. A compound having the structure of Formulae Ia, Ib, Ic, or Id: 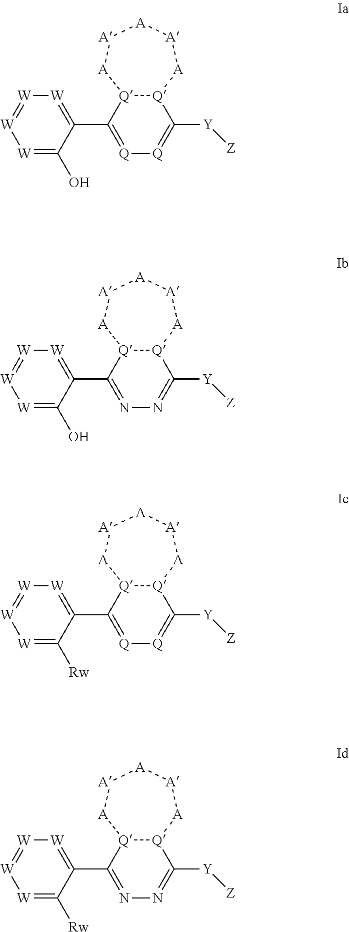
wherein:
R_{w} is hydrogen, C₁₋₆alkyl, C₂₋₈alkynyl, halogen, C₁₋₆alkoxy, halo-C₁₋₄alkyl, halo-C₁₋₄alkoxy, cyano, —NH₂, —N(C₁₋₆alkyl)₂, thiol, —SO₂NH₂, —SO₂N(C₁₋₆alkyl)₂, —S(═O)(C₁₋₆alkyl), cycloalkyl, CHR₁ₐ, (C═O)R₁ₐ, OR₁ₐ, N(R_{1b})₂, S(═O)R₁ₐ, S(═O)₂R₁ₐ, O(C═O)R₁ₐ, (C═O)OR₁ₐ, NR_{1b}(C═O)R_{1b}, (C═O)NHR_{1b}, (C═O)N(R_{1b})₂, NR_{1b}(C═O)OR₁ₐ, O(C═O)N(R_{1b})₂, ONR_{1b}(C═NR_{1b})NR_{1b}, NR_{1b}S(═O)₂R₁ₐ or S(═O)₂N(R_{1b})₂, wherein each C₁₋₆alkyl, heterocycle, heteroaryl and aryl are optionally substituted with 1 or 2 substituents each selected from R₅;
each W is independently CH, CR′ or N;
each Q is independently N or CH;
each Q′ is independently N, C or CH;
each A is independently CH, CH₂, CRₐ, CHRₐ, CR₄, CHR₄, N, NH, NRₐ, NR₄, S, or O;
each A′ is independently absent, CH, CH₂, CRₐ, CHRₐ, CR₄, CHR₄, N, NH, NRₐ, NR₄, S, or O;
each Rₐ is independently H, deuterium, halogen, —CN, —OH, —OR₂, ═O, ═N—OR₂, —SR₂, —S(═O)R₂, —S(═O)₂R², —N(R₂)₂, —NR₂S(═O)(═NR₂)R², —NR₂S(═O)₂R², —S(═O)₂N(R₂)₂, —C(═O)R₂, —OC(═O)R₂, —C(═O)OR₂, —OC(═O)OR₂, —C(═O)N(R₂)₂, —OC(═O)N(R₂)₂, —NR₂C(═O)R₂, —P(═O)(R₂)₂, C₁₋₄alkyl, (C₁₋₄alkyl)₂, halo-C₁₋₆alkyl, heteroalkyl, C₃₋₈cycloalkyl, C₂₋₇heterocycloalkyl, aryl or monocyclic heteroaryl;
each [symbol] represents either an absent, single, or double bond;
Y is C(R₁ₐ)₂, C═O, O, NR_{1b}, or a bond;
each R₁ₐ is independently hydrogen, halogen, hydroxyl, cyano, C₁₋₄alkyl, deutero-C₁₋₄alkyl, halo-C₁₋₄alkyl, amino, hydroxyC₁₋₄alkyl, C₃₋₁₀cycloalkyl, C₂₋₇heterocycloalkyl or aryl;
each R_{1b} is independently hydrogen, C₁₋₄alkyl, deutero-C₁₋₄alkyl, halo-C₁₋₄alkyl or hydroxyC₁₋₄alkyl;
each R′ is independently heterocyclyl, heteroaryl, aryl, C₃₋₈cycloalkyl, C₁₋₄alkyl, C₁₋₄alkoxy, deutero-C₁₋₄alkyl, halo-C₁₋₄alkyl, halo-C₁₋₄alkoxy, hydroxyC₁₋₄alkyl, halogen, hydroxy or cyano,
wherein each heterocycle, heteroaryl, C₃₋₈cycloalkyl and aryl are optionally substituted with 1, or 2 substituents each selected from R₃;
each Z is heterocyclyl, heteroaryl, aryl, C₃₋₁₀cycloalkyl, C₁₋₄alkyl, deutero-C₁₋₄alkyl, halo-C₁₋₄alkyl, hydroxy-C₁₋₈alkyl, C₁₋₆alkoxy, NH(hydroxy-C₁₋₆alkyl), NH(C₁₋₆alkoxy) wherein each Z is optionally substituted with OH, NH₂, —CO₂H, halogen, C₁₋₆alkyl, C₁₋₆haloalkyl, C₁₋₆ hydroxyalkyl, C₂₋₆acyl, C₂₋₆alkanoic acid, C₂₋₆alkanoate ester, or heterocyclyl, and wherein heterocyclyl, C₃₋₁₀cycloalkyl is a saturated or partially unsaturated 3-7 membered monocyclic, 6-10 membered bicyclic or 13-16 membered polycyclic ring system having 1, 2, or 3 heteroatom ring members independently selected from N, O, and S, each optionally substituted with 1, 2, 3, 4, or 5 substituents each selected from R₂;
R₂ is independently selected from halogen, hydroxyl, cyano, C₁₋₄alkyl, hydroxyC₁₋₄alkyl, deuterium-C₁₋₄alkyl, halo-C₁₋₄alkyl, amino, C₁₋₄alkyl-amino, (C₁₋₆alkyl)₂-amino, halo-C₁₋₄alkyl-amino, (halo-C₁₋₆alkyl)₂-amino, hydroxy-C₁₋₄alkyl-amino, C₁₋₄alkoxy-C₁₋₄alkyl-amino, amino-C₁₋₄alkyl, C₁₋₄alkyl-amino-C₁₋₄alkyl, (C₁₋₄alkyl-amino)₂-C₁₋₄alkyl, C₁₋₄alkoxy, halo-C₁₋₄alkoxy, hydroxy-C₁₋₄alkoxy, C₁₋₄alkyl-C₁₋₄alkoxy, C₃₋₁₀cycloalkyl, C₃₋₁₀cycloalkyl-amino, C₃₋₁₀cycloalkyl-amino-C₁₋₄alkyl, heteroaryl-C₁₋₄alkyl, heteroaryl-amino, heteroaryl-C₁₋₄alkyl-amino, heterocyclyl, heterocyclyl-C₁₋₄alkyl, heterocyclyl-amino, heterocyclyl-amino-C₁₋₄alkyl, heterocyclyl-C₁₋₄alkoxy, heterocyclyl-amino-C₃₋₁₀cycloalkyl, phenyl, and phenyl-C₁₋₄alkoxy,
wherein heteroaryl is a 5-6 membered monocyclic or 6-10 membered bicyclic ring system having 1, 2, 3, or 4 heteroatom ring members independently selected from N, O, or S,
wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S,
wherein C₃₋₁₀cycloalkyl is a saturated or partially unsaturated 3-7 membered monocyclic ring system, and
wherein each instance of phenyl, heteroaryl, heterocyclyl, or C₃₋₁₀cycloalkyl is optionally substituted with 1 or 2 substituents each selected from R₃;
R₃ is independently selected from halogen, hydroxyl, cyano, C₁₋₄alkyl, deutero-C₁₋₄alkyl, halo-C₁₋₄alkyl, amino, C₁₋₄alkoxy, and halo-C₁₋₄alkoxy;
each R₄ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, halo-C₁₋₄alkoxy, heteroaryl, heterocyclyl, and phenyl,
wherein heteroaryl is a 5-6 membered monocyclic or 9-10 membered bicyclic ring system having 1, 2, 3, or 4 heteroatom ring members selected from N, O, and S,
wherein heterocyclyl is a saturated or partially unsaturated 3-7 membered monocyclic or 9-10 membered bicyclic ring system having 1, 2, or 3 heteroatom ring members selected from N, O, and S, and
wherein each instance of phenyl, heteroaryl or heterocyclyl is optionally substituted with 1, or 2 substituents each selected from R₅;
R₅ is independently selected from halogen, hydroxyl, cyano, nitro, C₁₋₄alkyl, deutero-C₁₋₄alkyl, halo-C₁₋₄alkyl, amino, C₁₋₄alkyl-amino, (C₁₋₄alkyl)₂-amino, amino-C₁₋₄alkyl, hydroxyC₁₋₄alkyl, C₁₋₄alkyl-carbonyl, C₁₋₄alkoxy, C₁₋₄alkylthio, halo-C₁₋₄alkoxy, and C₃₋₁₀cycloalkyl;
with the proviso that the compound is not Phenol, 5-([1,1′-biphenyl]-4-yl-4-dibenzothienylamino)-2-[4-([1,1′-biphenyl]-4-yl-4-dibenzothienylamino)-1-naphthalenyl]; 5-[Phenyl(6-phenyl-4-dibenzothienyl)amino]-2-[4-[phenyl(6-phenyl-4-dibenzothienyl)amino]-1-naphthalenyl]phenol; 5-[2-Dibenzothienyl(9,9-dimethyl-9H-fluoren-2-yl)amino]-2-[4-[2-dibenzothienyl(9,9-dimethyl-9H-fluoren-2-yl)amino]-1-naphthalenyl]phenol; 5-[2-Dibenzofuranyl(9,9-dimethyl-9H-fluoren-2-yl)amino]-2-[4-[2-dibenzofuranyl(9,9-dimethyl-9H-fluoren-2-yl)amino]-1-naphthalenyl]phenol; 5-(4-Dibenzofuranylphenylamino)-2-[4-(4-dibenzofuranylphenylamino)-1-naphthalenyl]phenol; 5-(4-Dibenzofuranyl-2-naphthalenylamino)-2-[4-(4-dibenzofuranyl-2-naphthalenylamino)-1-naphthalenyl]phenol; 5-[Phenyl(6-phenyl-4-dibenzofuranyl)amino]-2-[4-[phenyl(6-phenyl-4-dibenzofuranyl)amino]-1-naphthalenyl]phenol; 5-([1,1′-Biphenyl]-2-yl-4-dibenzofuranylamino)-2-[4-([1,1′-biphenyl]-2-yl-4-dibenzofuranylamino)-1-naphthalenyl]phenol; 4-[4-(4-Dibenzofuranylphenylamino)-1-naphthalenyl]-2′,3′,5′,6′-tetrafluoro-4′-(2-naphthalenylphenylamino)[1,1′-biphenyl]-3-ol; 2-[4-[[6-[[3-(2-Amino-4-pyrimidinyl)-2-pyridinyl]oxy]-3-pyridinyl]amino]-1-phthalazinyl]phenol; or 2-[4-[[6-[[3-(2-Amino-4-pyrimidinyl)-2-pyridinyl]oxy]-3-pyridinyl]amino]-1-phthalazinyl]-4-fluorophenol; wherein a form of the compound is selected from the group consisting of a pharmaceutically acceptable salt, hydrate, solvate, racemate, enantiomer, diastereomer, stereoisomer, tautomer, and isotope enriched form thereof.

2. The compound of claim 1 having the structure of Formulae IIa, IIb, IIc, or IId: 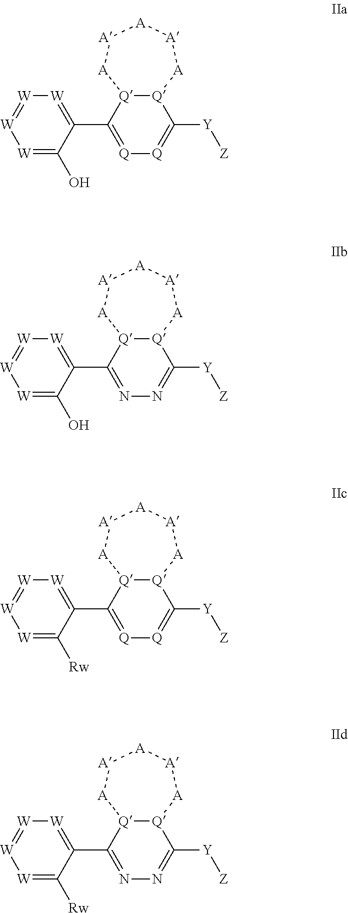
wherein:
R_{w} is hydrogen, C₁₋₆alkyl, C₂₋₈alkynyl, halogen, C₁₋₆alkoxy, halo-C₁₋₄alkyl, halo-C₁₋₄alkoxy, cyano, —NH₂, —N(C₁₋₆alkyl)₂, thiol, —SO₂NH₂, —SO₂N(C₁₋₆alkyl)₂, —S(═O)(C₁₋₆alkyl), cycloalkyl, CHR₁ₐ, (C═O)R₁ₐ, OR₁ₐ, N(R_{1b})₂, S(═O)R₁ₐ, S(═O)₂R₁ₐ, O(C═O)R₁ₐ, (C═O)OR₁ₐ, NR_{1b}(C═O)R_{1b}, (C═O)NHR_{1b}, (C═O)N(R_{1b})₂ NR_{1b}(C═O)OR₁ₐ, O(C═O)N(R_{1b})₂, ONR_{1b}(C═NR_{1b})NR_{1b}, NR_{1b}S(═O)₂R₁ₐ and S(═O)₂N(R_{1b})₂, wherein each C₁₋₆alkyl, heterocycle, heteroaryl and aryl are optionally substituted with 1 or 2 substituents each selected from R₅;
each W is independently CH, CR′, or N, wherein at least one Q′ is N;
each Q is independently N or CH;
each Q′ is independently N, C or CH;
each A is independently CH, CH₂, CRₐ, CHRₐ, CR₄, CHR₄, N, NH, NRₐ, NR₄, S, or O;
each A′ is independently absent, CH, CH₂, CRₐ, CHRₐ, CR₄, CHR₄, N, NH, NRₐ, NR₄, S, or O;
each Rₐ is independently H, deuterium, halogen, —CN, —OH, —OR₂, ═O, ═N—OR₂, —SR₂, —S(═O)R₂, —S(═O)₂R², —N(R₂)₂, —NR₂S(═O)(═NR₂)R², —NR₂S(═O)₂R², —S(═O)₂N(R₂)₂, —C(═O)R₂, —OC(═O)R₂, —C(═O)OR₂, —OC(═O)OR₂, —C(═O)N(R₂)₂, —OC(═O)N(R₂)₂, —NR₂C(═O)R₂, —P(═O)(R₂)₂, C₁₋₄alkyl, (C₁₋₄alkyl)₂, halo-C₁₋₆alkyl, C₁₋₆heteroalkyl, C₃₋₈cycloalkyl, C₂₋₇heterocycloalkyl, aryl or monocyclic heteroaryl;
each [symbol] represents either an absent, single, or double bond;
Y is C(R₁ₐ)₂, C═O, O, NR_{1b} or a bond;
each R₁ₐ is independently hydrogen, halogen, hydroxyl, cyano, C₁₋₄alkyl, deutero-C₁₋₄alkyl, halo-C₁₋₄alkyl, amino hydroxyC₁₋₄alkyl, C₃₋₁₀cycloalkyl, C₂₋₇heterocycloalkyl or aryl;
each R_{1b} is independently hydrogen, C₁₋₄alkyl, deutero-C₁₋₄alkyl, halo-C₁₋₄alkyl or hydroxyC₁₋₄alkyl;
each R′ is independently heterocyclyl, heteroaryl, aryl, C₃₋₈cycloalkyl, C₁₋₄alkyl, C₁₋₄alkoxy, deutero-C₁₋₄alkyl, halo-C₁₋₄alkyl, halo-C₁₋₄alkoxy, hydroxyC₁₋₄alkyl, halogen, hydroxy or cyano,
wherein each heterocycle, heteroaryl, C₃₋₈cycloalkyl and aryl are optionally substituted with 1, or 2, substituents each selected from R₃;
each Z is heterocyclyl, heteroaryl, aryl, C₃₋₁₀cycloalkyl, C₁₋₄alkyl, deutero-C₁₋₄alkyl, halo-C₁₋₄alkyl, hydroxy-C₁₋₈alkyl, C₁₋₆alkoxy, NH(₁₋₆alkyl), NH(C₁₋₆alkoxy) wherein each Z is optionally substituted with OH, NH₂, —CO₂H, halogen, C₁₋₆alkyl, C₁₋₆haloalkyl, C₁₋₆ hydroxyalkyl, C₂₋₆acyl, C₂₋₆alkanoic acid, C₂₋₆alkanoate ester, or heterocyclyl, and wherein heterocyclyl, C₃₋₁₀cycloalkyl a saturated or partially unsaturated 3-7 membered monocyclic, 6-10 membered bicyclic or 13-16 membered polycyclic ring system having 1, 2, or 3 heteroatom ring members independently selected from N, O, and S, each optionally substituted with 1, 2, 3, 4, or 5 substituents each selected from R₂;
R₂ is independently selected from halogen, hydroxyl, cyano, C₁₋₄alkyl, hydroxyC₁₋₄alkyl, deutero-C₁₋₄alkyl, halo-C₁₋₄alkyl, amino, C₁₋₄alkyl-amino, (C₁₋₆alkyl)₂-amino, halo-C₁₋₄alkyl-amino, (halo-C₁₋₆alkyl)₂-amino, hydroxy-C₁₋₄alkyl-amino, C₁₋₄alkoxy-C₁₋₄alkyl-amino, amino-C₁₋₄alkyl, C₁₋₄alkyl-amino-C₁₋₄alkyl, (C₁₋₄alkyl-amino)₂-C₁₋₄alkyl, C₁₋₄alkoxy, halo-C₁₋₄alkoxy, ₁₋₄alkoxy, C₁₋₄alkyl-C₁₋₄alkoxy, C₃₋₁₀cycloalkyl, C₃₋₁₀cycloalkyl-amino, C₃₋₁₀cycloalkyl-amino-C₁₋₄alkyl, heteroaryl-C₁₋₄alkyl, heteroaryl-amino, heteroaryl-C₁₋₄alkyl-amino, heterocyclyl, heterocyclyl-C₁₋₄alkyl, heterocyclyl-amino, heterocyclyl-amino-C₁₋₄alkyl, heterocyclyl-C₁₋₄alkoxy, heterocyclyl-amino-C₃₋₁₀cycloalkyl, phenyl, and phenyl-C₁₋₄alkoxy,
wherein heteroaryl is a 5-6 membered monocyclic or 6-10 membered bicyclic ring system having 1, 2, 3, or 4 heteroatom ring members independently selected from N, O, and S,
wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S,
wherein C₃₋₁₀cycloalkyl is a saturated or partially unsaturated 3-7 membered monocyclic ring system, and
wherein each instance of phenyl, heteroaryl, heterocyclyl, or C₃₋₁₀cycloalkyl is optionally substituted with 1 or 2 substituents each selected from R₃;
R₃ is independently selected from halogen, hydroxyl, cyano, C₁₋₄alkyl, deutero-C₁₋₄alkyl, halo-C₁₋₄alkyl, amino, C₁₋₄alkoxy, and halo-C₁₋₄alkoxy;
each R₄ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, halo-C₁₋₄alkoxy, heteroaryl, heterocyclyl, and phenyl,
wherein heteroaryl is a 5-6 membered monocyclic or 9-10 membered bicyclic ring system having 1, 2, 3, or 4 heteroatom ring members selected from N, O, and S,
wherein heterocyclyl is a saturated or partially unsaturated₃₋₆ membered monocyclic or 9-10 membered bicyclic ring system having 1, 2, or 3 heteroatom ring members selected from N, O, and S, and
wherein each instance of phenyl, heteroaryl or heterocyclyl is optionally substituted with 1, or 2 substituents each selected from R₅; and
R₅ is independently selected from halogen, hydroxyl, cyano, nitro, C₁₋₄alkyl, deutero-C₁₋₄alkyl, halo-C₁₋₄alkyl, amino, C₁₋₄alkyl-amino, (C₁₋₄alkyl)₂-amino, amino-C₁₋₄alkyl, hydroxyC₁₋₄alkyl, C₁₋₄alkyl-carbonyl, C₁₋₄alkoxy, C₁₋₄alkylthio, halo-C₁₋₄alkoxy, and C₃₋₁₀cycloalkyl;
wherein a form of the compound is selected from the group consisting of a pharmaceutically acceptable salt, hydrate, solvate, racemate, enantiomer, diastereomer, stereoisomer, tautomer, and isotope enriched form thereof.

3. The compound of claim 1 having the structure of Formulae IIIa, IIIb, IIIc, or IIId: 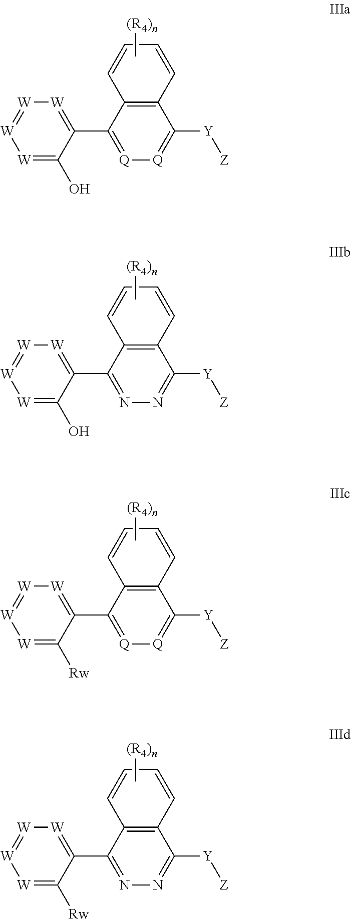
wherein:
R_{w} is hydrogen, C₁₋₆alkyl, C₂₋₈alkynyl, halogen, C₁₋₆alkoxy, halo-C₁₋₄alkyl, halo-C₁₋₄alkoxy, cyano, —NH₂, —N(C₁₋₆alkyl)₂, thiol, —SO₂NH₂, —SO₂N(C₁₋₆alkyl)₂, —S(═O)(C₁₋₆alkyl), cycloalkyl, CHR₁ₐ, (C═O)R₁ₐ, OR₁ₐ, N(R_{1b})₂, S(═O)R₁ₐ, S(═O)₂R₁ₐ, O(C═O)R₁ₐ, (C═O)OR₁ₐ, NR_{1b}(C═O)R_{1b}, (C═O)NHR_{1b}, (C═O)N(R_{1b})₂ NR_{1b}(C═O)OR₁ₐ, O(C═O)N(R_{1b})₂, ONR_{1b}(C═NR_{1b})NR_{1b}, NR_{1b}S(═O)₂R₁ₐ and S(═O)₂N(R_{1b})₂, wherein each heterocycle, heteroaryl and aryl are optionally substituted with 1 or 2 substituents each selected from R₅;
each W is independently CH, CR′ or N;
each Q is independently N, or CH;
Y is C(R₁ₐ)₂, C═O, O, NR_{1b} or a bond;
each R₁ₐ is independently hydrogen, halogen, hydroxyl, cyano, C₁₋₄alkyl, deutero-C₁₋₄alkyl, halo-C₁₋₄alkyl, amino or hydroxyC₁₋₄alkyl;
each R_{1b} is independently hydrogen, C₁₋₄alkyl, deutero-C₁₋₄alkyl, halo-C₁₋₄alkyl or hydroxyC₁₋₄alkyl;
each R₄ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, halo-C₁₋₄alkoxy, heteroaryl, heterocyclyl, and phenyl,
wherein heteroaryl is a 5-6 membered monocyclic or 9-10 membered bicyclic ring system having 1, 2, 3, or 4 heteroatom ring members selected from N, O, and S,
wherein heterocyclyl is a saturated or partially unsaturated 3-6 membered monocyclic or 9-10 membered bicyclic ring system having 1, 2, or 3 heteroatom ring members selected from N, O, and S, and
wherein each instance of phenyl, heteroaryl or heterocyclyl is optionally substituted with 1, or 2 substituents each selected from R₅;
each Z is heterocyclyl, heteroaryl, aryl, C₃₋₁₀cycloalkyl, C₁₋₄alkyl, deutero-C₁₋₄alkyl, halo-C₁₋₄alkyl, hydroxy-C₁₋₈alkyl, NH(hydroxy-C₁₋₆alkyl), NH(C₁₋₆alkoxy) wherein each Z is optionally substituted with OH, NH₂, —CO₂H, halogen, C₁₋₆alkyl, C₁₋₆haloalkyl, C₁₋₆ hydroxyalkyl, C₂₋₆acyl, C₂₋₆alkanoic acid, C₂₋₆alkanoate ester, or heterocyclyl, wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S, each optionally substituted with 1, 2, 3, 4, or 5 substituents each selected from R₂;
R₂ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₆alkyl)₂-amino, halo-C₁₋₄alkyl-amino, (halo-C₁₋₆alkyl)₂-amino, hydroxy-C₁₋₄alkyl-amino, C₁₋₄alkoxy-C₁₋₄alkyl-amino, amino-C₁₋₄alkyl, C₁₋₄alkyl-amino-C₁₋₄alkyl, (C₁₋₄alkyl-amino)₂-C₁₋₄alkyl, C₁₋₄alkoxy, halo-C₁₋₄alkoxy, hydroxy-C₁₋₄alkoxy, C₁₋₄alkyl-C₁₋₄alkoxy, C₃₋₁₀cycloalkyl, C₃₋₁₀cycloalkyl-amino, C₃₋₁₀cycloalkyl-amino-C₁₋₄alkyl, heteroaryl-C₁₋₄alkyl, heteroaryl-amino, heteroaryl-C₁₋₄alkyl-amino, heterocyclyl, heterocyclyl-C₁₋₄alkyl, heterocyclyl-amino, heterocyclyl-amino-C₁₋₄alkyl, heterocyclyl-C₁₋₄alkoxy, heterocyclyl-amino-C₃₋₁₀cycloalkyl, phenyl, and phenyl-C₁₋₄alkoxy,
wherein heteroaryl is a 5-6 membered monocyclic or 6-10 membered bicyclic ring system having 1, 2, 3, or 4 heteroatom ring members independently selected from N, O, and S,
wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S,
wherein C₃₋₁₀cycloalkyl is a saturated or partially unsaturated 3-7 membered monocyclic ring system, and
wherein each instance of phenyl, heteroaryl, heterocyclyl, or C₃₋₁₀cycloalkyl is optionally substituted with 1 or 2 substituents each selected from R₃;
R₃ is independently selected from halogen, hydroxyl, cyano, C₁₋₄alkyl, deutero-C₁₋₄alkyl, halo-C₁₋₄alkyl, amino, C₁₋₄alkoxy, and halo-C₁₋₄alkoxy;
each R′ and R₄ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, halo-C₁₋₄alkoxy, heteroaryl, heterocyclyl, and phenyl,
wherein heteroaryl is a 5-6 membered monocyclic or 9-10 membered bicyclic ring system having 1, 2, 3, or 4 heteroatom ring members selected from N, O, and S,
wherein heterocyclyl is a saturated or partially unsaturated 3-6 membered monocyclic or 9-10 membered bicyclic ring system having 1, 2, or 3 heteroatom ring members selected from N, O, and S,
wherein each instance of phenyl, heteroaryl or heterocyclyl is optionally substituted with 1, or 2 substituents each selected from R₅;
R₅ is independently selected from halogen, hydroxyl, cyano, nitro, C₁₋₄alkyl, deutero-C₁₋₄alkyl, halo-C₁₋₄alkyl, amino, C₁₋₄alkyl-amino, (C₁₋₄alkyl)₂-amino, amino-C₁₋₄alkyl, hydroxyC₁₋₄alkyl, C₁₋₄alkyl-carbonyl, C₁₋₄alkoxy, C₁₋₄alkylthio, halo-C₁₋₄alkoxy, and C₃₋₁₀cycloalkyl; and
n is 0, 1, or 2;
wherein a form of the compound is selected from the group consisting of a pharmaceutically acceptable salt, hydrate, solvate, racemate, enantiomer, diastereomer, stereoisomer, tautomer, and isotope enriched form thereof.

4. The compound of claim 1 having the structure of Formulae IVa, IVb, IVc, or IVd: 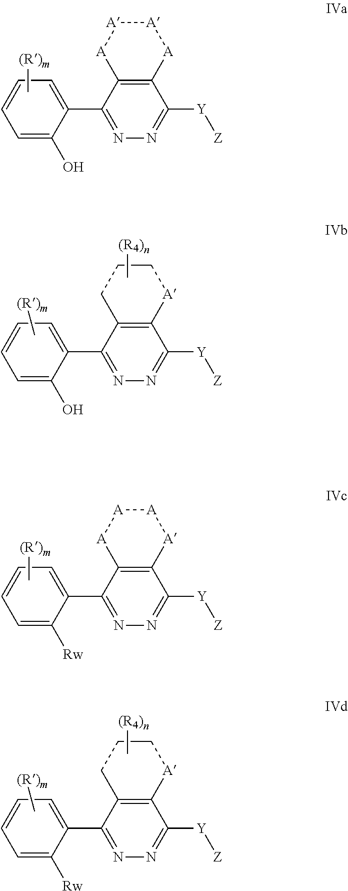
wherein:
R_{w} is H, C₁₋₆alkyl, C₂₋₈alkynyl, halogen, C₁₋₆alkoxy, halo-C₁₋₄alkyl, halo-C₁₋₄alkoxy, cyano, —NH₂, —N(C₁₋₆alkyl)₂, thiol, —SO₂NH₂, —SO₂N(C₁₋₆alkyl)₂, —S(═O)(C₁₋₆alkyl), cycloalkyl, CHR₁ₐ, (C═O)R₁ₐ, OR₁ₐ, N(R_{1b})₂, S(═O)R₁ₐ, S(═O)₂R₁ₐ, O(C═O)R₁ₐ, (C═O)OR₁ₐ, NR_{1b}(C═O)R_{1b}, (C═O)NHR_{1b}, (C═O)N(R_{1b})₂ NR_{1b}(C═O)OR₁ₐ, O(C═O)N(R_{1b})₂, ONR_{1b}(C═NR_{1b})NR_{1b}, NR_{1b}S(═O)₂R₁ₐ or S(═O)₂N(R_{1b})₂, wherein each heterocycle, heteroaryl and aryl are optionally substituted with 1 or 2 substituents each selected from R₅;
each A is independently NH, S, O, CH₂, or CR₄;
A′ is independently NH, S, O, CH₂, CR₄ or absent;
Each [symbol] represents either an absent, single, or double bond;
Y is C(R₁ₐ)₂, C═O, O, NR_{1b} or a bond;
R₁ₐ is hydrogen, halogen, hydroxyl, cyano, C₁₋₄alkyl, deutero-C₁₋₄alkyl, halo-C₁₋₄alkyl, amino or hydroxyC₁₋₄alkyl;
R_{1b} is hydrogen, C₁₋₄alkyl, deutero-C₁₋₄alkyl, halo-C₁₋₄alkyl or hydroxyC₁₋₄alkyl; and
each Z is heterocyclyl, heteroaryl, aryl, C₃₋₁₀cycloalkyl, C₁₋₄alkyl, deutero-C₁₋₄alkyl, halo-C₁₋₄alkyl, hydroxy-C₁₋₈alkyl, NH(hydroxy-C₁₋₆alkyl), NH(C₁₋₆alkoxy) wherein each Z is optionally substituted with OH, NH₂, —CO₂H, halogen, C₁₋₆alkyl, C₁₋₆haloalkyl, C₁₋₆ hydroxyalkyl, C₂₋₆acyl, C₂₋₆alkanoic acid, C₂₋₆alkanoate ester, or heterocyclyl, wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S, each optionally substituted with 1, 2, 3, 4, or 5 substituents each selected from R₂;
R₂ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₆alkyl)₂-amino, halo-C₁₋₄alkyl-amino, (halo-C₁₋₆alkyl)₂-amino, hydroxy-C₁₋₄alkyl-amino, C₁₋₄alkoxy-C₁₋₄alkyl-amino, amino-C₁₋₄alkyl, C₁₋₄alkyl-amino-C₁₋₄alkyl, (C₁₋₄alkyl-amino)₂-C₁₋₄alkyl, C₁₋₄alkoxy, halo-C₁₋₄alkoxy, hydroxy-C₁₋₄alkoxy, C₁₋₄alkyl-C₁₋₄alkoxy, C₃₋₁₀cycloalkyl, C₃₋₁₀cycloalkyl-amino, C₃₋₁₀cycloalkyl-amino-C₁₋₄alkyl, heteroaryl-C₁₋₄alkyl, heteroaryl-amino, heteroaryl-C₁₋₄alkyl-amino, heterocyclyl, heterocyclyl-C₁₋₄alkyl, heterocyclyl-amino, heterocyclyl-amino-C₁₋₄alkyl, heterocyclyl-C₁₋₄alkoxy, heterocyclyl-amino-C₃₋₁₀cycloalkyl, phenyl, and phenyl-C₁₋₄alkoxy,
wherein heteroaryl is a 5-6 membered monocyclic or 6-10 membered bicyclic ring system having 1, 2, 3, or 4 heteroatom ring members independently selected from N, O, or S,
wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S,
wherein C₃₋₁₀cycloalkyl is a saturated or partially unsaturated 3-7 membered monocyclic ring system, and
wherein each instance of phenyl, heteroaryl, heterocyclyl, or C₃₋₁₀cycloalkyl is optionally substituted with 1 or 2 substituents each selected from R₃;
R₃ is independently selected from halogen, hydroxyl, cyano, C₁₋₄alkyl, deutero-C₁₋₄alkyl, halo-C₁₋₄alkyl, amino, C₁₋₄alkoxy, and halo-C₁₋₄alkoxy;
each R₄ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, halo-C₁₋₄alkoxy, heteroaryl, heterocycle, and phenyl,
wherein heteroaryl is a 5-6 membered monocyclic or 9-10 membered bicyclic ring system having 1, 2, 3, or 4 heteroatom ring members selected from N, O, and S,
wherein heterocyclyl is a saturated or partially unsaturated 3-6 membered monocyclic or 9-10 membered bicyclic ring system having 1, 2, or 3 heteroatom ring members selected from N, O, and S, and
wherein each instance of phenyl, heteroaryl or heterocyclyl is optionally substituted with 1, or 2 substituents each selected from R₅;
R₅ is independently selected from halogen, hydroxyl, cyano, nitro, C₁₋₄alkyl, deutero-C₁₋₄alkyl, halo-C₁₋₄alkyl, amino, C₁₋₄alkyl-amino, (C₁₋₄alkyl)₂-amino, amino-C₁₋₄alkyl, hydroxyC₁₋₄alkyl, C₁₋₄alkyl-carbonyl, C₁₋₄alkoxy, C₁₋₄alkylthio, halo-C₁₋₄alkoxy, and C₃₋₁₀cycloalkyl;
m is 0, 1, 2, 3, or 4; and
n is 0, 1, 2, or 3;
wherein a form of the compound is selected from the group consisting of a pharmaceutically acceptable salt, hydrate, solvate, racemate, enantiomer, diastereomer, stereoisomer, tautomer, and isotope enriched form thereof.

5. The compound of claim 1 having the structure of Formulae Va, Vb, Vc, or Vd: 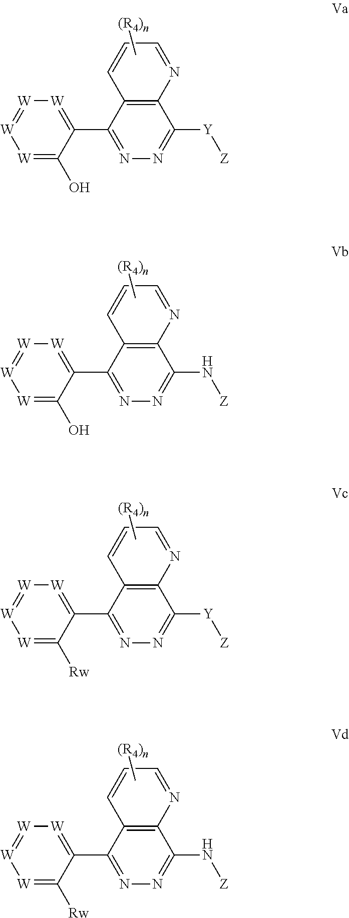
wherein:
R_{w} is hydrogen, C₁₋₆alkyl, C₂₋₈alkynyl, halogen, C₁₋₆alkoxy, halo-C₁₋₄alkyl, halo-C₁₋₄alkoxy, cyano, —NH₂, —N(C₁₋₆alkyl)₂, thiol, —SO₂NH₂, —SO₂N(C₁₋₆alkyl)₂, —S(═O)(C₁₋₆alkyl), cycloalkyl, CHR₁ₐ, (C═O)R₁ₐ, OR₁ₐ, N(R_{1b})₂, S(═O)R₁ₐ, S(═O)₂R₁ₐ, O(C═O)R₁ₐ, (C═O)OR₁ₐ, NR_{1b}(C═O)R_{1b}, (C═O)NHR_{1b}, (C═O)N(R_{1b})₂ NR_{1b}(C═O)OR₁ₐ, O(C═O)N(R_{1b})₂, ONR_{1b}(C═NR_{1b})NR_{1b}, NR_{1b}S(═O)₂R₁ₐ or S(═O)₂N(R_{1b})₂, wherein each heterocycle, heteroaryl and aryl are optionally substituted with 1 or 2 substituents each selected from R₅;
each W is independently CH, CR′, or N;
Y is C(R₁ₐ)₂, C═O, O, NR_{1b} or a bond;
R₁ₐ is hydrogen, halogen, hydroxyl, cyano, C₁₋₄alkyl, deutero-C₁₋₄alkyl, halo-C₁₋₄alkyl, amino or hydroxyC₁₋₄alkyl;
R_{1b} is hydrogen, C₁₋₄alkyl, deutero-C₁₋₄alkyl, halo-C₁₋₄alkyl or hydroxyC₁₋₄alkyl; and
each Z is heterocyclyl, heteroaryl, aryl, C₃₋₁₀cycloalkyl, C₁₋₄alkyl, deutero-C₁₋₄alkyl, halo-C₁₋₄alkyl, hydroxy-C₁₋₈alkyl, NH(hydroxy-C₁₋₆alkyl), NH(C₁₋₆alkoxy) wherein each Z is optionally substituted with OH, NH₂, —CO₂H, halogen, C₁₋₆alkyl, C₁₋₆haloalkyl, C₁₋₆ hydroxyalkyl, C₂₋₆acyl, C₂₋₆alkanoic acid, C₂₋₆alkanoate ester, or heterocyclyl, wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S, each optionally substituted with 1, 2, 3, 4, or 5 substituents each selected from R₂;
R₂ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₆alkyl)₂-amino, halo-C₁₋₄alkyl-amino, (halo-C₁₋₆alkyl)₂-amino, hydroxy-C₁₋₄alkyl-amino, C₁₋₄alkoxy-C₁₋₄alkyl-amino, amino-C₁₋₄alkyl, C₁₋₄alkyl-amino-C₁₋₄alkyl, (C₁₋₄alkyl-amino)₂-C₁₋₄alkyl, C₁₋₄alkoxy, halo-C₁₋₄alkoxy, hydroxy-C₁₋₄alkoxy, C₁₋₄alkyl-C₁₋₄alkoxy, C₃₋₁₀cycloalkyl, C₃₋₁₀cycloalkyl-amino, C₃₋₁₀cycloalkyl-amino-C₁₋₄alkyl, heteroaryl-C₁₋₄alkyl, heteroaryl-amino, heteroaryl-C₁₋₄alkyl-amino, heterocyclyl, heterocyclyl-C₁₋₄alkyl, heterocyclyl-amino, heterocyclyl-amino-C₁₋₄alkyl, heterocyclyl-C₁₋₄alkoxy, heterocyclyl-amino-C₃₋₁₀cycloalkyl, phenyl, and phenyl-C₁₋₄alkoxy,
wherein heteroaryl is a 5-6 membered monocyclic or 6-10 membered bicyclic ring system having 1, 2, 3, or 4 heteroatom ring members independently selected from N, O, and S,
wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S,
wherein C₃₋₁₀cycloalkyl is a saturated or partially unsaturated 3-7 membered monocyclic ring system,
wherein each instance of phenyl, heteroaryl, heterocyclyl, or C₃₋₁₀cycloalkyl is optionally substituted with 1 or 2 substituents each selected from R₃;
R₃ is independently selected from halogen, hydroxyl, cyano, C₁₋₄alkyl, deutero-C₁₋₄alkyl, halo-C₁₋₄alkyl, amino, C₁₋₄alkoxy, and halo-C₁₋₄alkoxy;
each R′ and R₄ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, halo-C₁₋₄alkoxy, heteroaryl, heterocyclyl, and phenyl,
wherein heteroaryl is a 5-6 membered monocyclic or 9-10 membered bicyclic ring system having 1, 2, 3, or 4 heteroatom ring members selected from N, O, and S,
wherein heterocyclyl is a saturated or partially unsaturated 3-6 membered monocyclic or 9-10 membered bicyclic ring system having 1, 2, or 3 heteroatom ring members selected from N, O, and S, and
wherein each instance of phenyl, heteroaryl or heterocyclyl is optionally substituted with 1, or 2 substituents each selected from R₅;
R₅ is independently selected from halogen, hydroxyl, cyano, nitro, C₁₋₄alkyl, deutero-C₁₋₄alkyl, halo-C₁₋₄alkyl, amino, C₁₋₄alkyl-amino, (C₁₋₄alkyl)₂-amino, amino-C₁₋₄alkyl, hydroxyC₁₋₄alkyl, C₁₋₄alkyl-carbonyl, C₁₋₄alkoxy, C₁₋₄alkylthio, halo-C₁₋₄alkoxy, and C₃₋₁₀cycloalkyl; and
n is 0, 1, 2, or 3;
wherein a form of the compound is selected from the group consisting of a pharmaceutically acceptable salt, hydrate, solvate, racemate, enantiomer, diastereomer, stereoisomer, tautomer, and isotope enriched form thereof.

6. The compound of claim 1 having the structure of Formulae VIa, VIb, VIc, or VId: 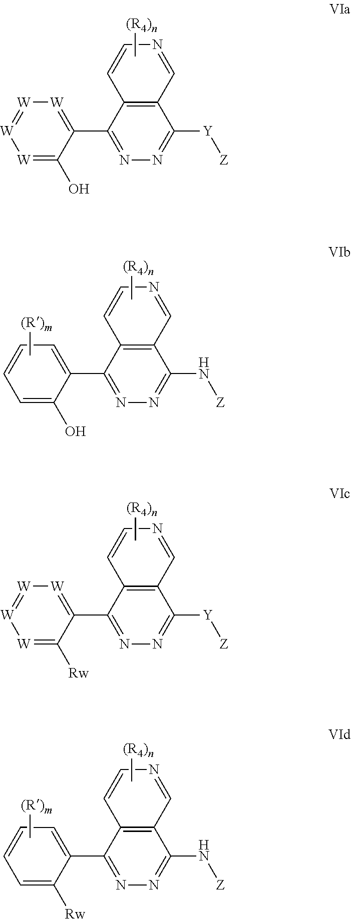
wherein:
R_{w} is hydrogen, C₁₋₆alkyl, C₂₋₈alkynyl, halogen, C₁₋₆alkoxy, halo-C₁₋₄alkyl, halo-C₁₋₄alkoxy, cyano, —NH₂, —N(C₁₋₆alkyl)₂, thiol, —SO₂NH₂, —SO₂N(C₁₋₆alkyl)₂, —S(═O)(C₁₋₆alkyl), cycloalkyl, CHR₁ₐ, (C═O)R₁ₐ, OR₁ₐ, N(R_{1b})₂, S(═O)R₁ₐ, S(═O)₂R₁ₐ, O(C═O)R₁ₐ, (C═O)OR₁ₐ, NR_{1b}(C═O)R_{1b}, (C═O)NHR_{1b}, (C═O)N(R_{1b})₂ NR_{1b}(C═O)OR₁ₐ, O(C═O)NHR_{1b}, ONR_{1b}(C═NR_{1b})NR_{1b}, NR_{1b}S(═O)₂R₁ₐ or S(═O)₂N(R_{1b})₂, wherein each heterocycle, heteroaryl and aryl are optionally substituted with 1 or 2 substituents each selected from R₅;
each W is independently CH, CR′, or N;
Y is C(R₁ₐ)₂, C═O, O, NR_{1b} or a bond;
each R₁ₐ is independently hydrogen, halogen, hydroxyl, cyano, C₁₋₄alkyl, deutero-C₁₋₄alkyl, halo-C₁₋₄alkyl, amino or hydroxyC₁₋₄alkyl;
each R_{1b} is independently hydrogen, C₁₋₄alkyl, deutero-C₁₋₄alkyl, halo-C₁₋₄alkyl or hydroxyC₁₋₄alkyl;
each Z is heterocyclyl, heteroaryl, aryl, C₃₋₁₀cycloalkyl, C₁₋₄alkyl, deutero-C₁₋₄alkyl, halo-C₁₋₄alkyl, hydroxy-C₁₋₈alkyl, NH(hydroxy-C₁₋₆alkyl), NH(C₁₋₆alkoxy) wherein each Z is optionally substituted with OH, NH₂, —CO₂H, halogen, C₁₋₆alkyl, C₁₋₆haloalkyl, C₁₋₆ hydroxyalkyl, C₂₋₆acyl, C₂₋₆alkanoic acid, C₂₋₆alkanoate ester, or heterocyclyl, wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S, each optionally substituted with 1, 2, 3, 4, or 5 substituents each selected from R₂;
R₂ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₆alkyl)₂-amino, halo-C₁₋₄alkyl-amino, (halo-C₁₋₆alkyl)₂-amino, hydroxy-C₁₋₄alkyl-amino, C₁₋₄alkoxy-C₁₋₄alkyl-amino, amino-C₁₋₄alkyl, C₁₋₄alkyl-amino-C₁₋₄alkyl, (C₁₋₄alkyl-amino)₂-C₁₋₄alkyl, C₁₋₄alkoxy, halo-C₁₋₄alkoxy, hydroxy-C₁₋₄alkoxy, C₁₋₄alkyl-C₁₋₄alkoxy, C₃₋₁₀cycloalkyl, C₃₋₁₀cycloalkyl-amino, C₃₋₁₀cycloalkyl-amino-C₁₋₄alkyl, heteroaryl-C₁₋₄alkyl, heteroaryl-amino, heteroaryl-C₁₋₄alkyl-amino, heterocyclyl, heterocyclyl-C₁₋₄alkyl, heterocyclyl-amino, heterocyclyl-amino-C₁₋₄alkyl, heterocyclyl-C₁₋₄alkoxy, heterocyclyl-amino-C₃₋₁₀cycloalkyl, phenyl, and phenyl-C₁₋₄alkoxy,
wherein heteroaryl is a 5-6 membered monocyclic or 6-10 membered bicyclic ring system having 1, 2, 3, or 4 heteroatom ring members independently selected from N, O, and S,
wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S,
wherein C₃₋₁₀cycloalkyl is a saturated or partially unsaturated 3-7 membered monocyclic ring system, and
wherein each instance of phenyl, heteroaryl, heterocyclyl, or C₃₋₁₀cycloalkyl is optionally substituted with 1 or 2 substituents each selected from R₃;
R₃ is independently selected from halogen, hydroxyl, cyano, C₁₋₄alkyl, deutero-C₁₋₄alkyl, halo-C₁₋₄alkyl, amino, C₁₋₄alkoxy, and halo-C₁₋₄alkoxy;
each R′ and R₄ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, halo-C₁₋₄alkoxy, heteroaryl, heterocyclyl, and phenyl,
wherein heteroaryl is a 5-6 membered monocyclic or 9-10 membered bicyclic ring system having 1, 2, 3, or 4 heteroatom ring members selected from N, O, and S,
wherein heterocyclyl is a saturated or partially unsaturated 3-6 membered monocyclic or 9-10 membered bicyclic ring system having 1, 2, or 3 heteroatom ring members selected from N, O, and S, and
wherein each instance of phenyl, heteroaryl or heterocyclyl is optionally substituted with 1, or 2 substituents each selected from R₅;
R₅ is independently selected from halogen, hydroxyl, cyano, nitro, C₁₋₄alkyl, deutero-C₁₋₄alkyl, halo-C₁₋₄alkyl, amino, C₁₋₄alkyl-amino, (C₁₋₄alkyl)₂-amino, amino-C₁₋₄alkyl, hydroxyC₁₋₄alkyl, C₁₋₄alkyl-carbonyl, C₁₋₄alkoxy, C₁₋₄alkylthio, halo-C₁₋₄alkoxy, and C₃₋₁₀cycloalkyl; and
n is 0, 1, 2, or 3;
wherein a form of the compound is selected from the group consisting of a pharmaceutically acceptable salt, hydrate, solvate, racemate, enantiomer, diastereomer, stereoisomer, tautomer, and isotope enriched form thereof.

7. The compound of claim 1 having the structure of Formulae VIIa, VIIb, VIIc, or VIId: 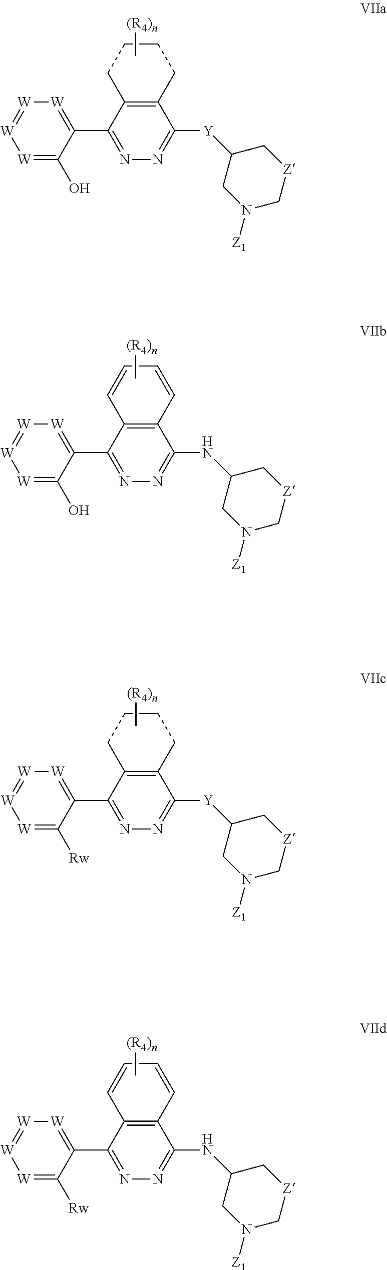
wherein:
R_{w} is hydrogen, C₁₋₆alkyl, C₂₋₈alkynyl, halogen, C₁₋₆alkoxy, halo-C₁₋₄alkyl, halo-C₁₋₄alkoxy, cyano, —NH₂, —N(C₁₋₆alkyl)₂, thiol, —SO₂NH₂, —SO₂N(C₁₋₆alkyl)₂, —S(═O)(C₁₋₆alkyl), cycloalkyl, CHR₁ₐ, (C═O)R₁ₐ, OR₁ₐ, N(R_{1b})₂, S(═O)R₁ₐ, S(═O)₂R₁ₐ, O(C═O)R₁ₐ, (C═O)OR₁ₐ, NR_{1b}(C═O)R_{1b}, (C═O)NHR_{1b}, (C═O)N(R_{1b})₂ NR_{1b}(C═O)OR₁ₐ, O(C═O)N(R_{1b})₂, ONR_{1b}(C═NR_{1b})NR_{1b}, NR_{1b}S(═O)₂R₁ₐ or S(═O)₂N(R_{1b})₂, wherein each heterocycle, heteroaryl and aryl are optionally substituted with 1 or 2 substituents each selected from R₅;
each W is independently CH, CR′ or N;
each [symbol] represents either an absent, single, or double bond;
each Z′ is independently CH₂ or absent;
Y is C(R₁ₐ)₂, C═O, O, NR_{1b} or a bond;
each R₁ₐ is independently hydrogen, halogen, hydroxyl, cyano, C₁₋₄alkyl, deutero-C₁₋₄alkyl, halo-C₁₋₄alkyl, amino or hydroxyC₁₋₄alkyl;
each R_{1b} is independently hydrogen, C₁₋₄alkyl, deutero-C₁₋₄alkyl, halo-C₁₋₄alkyl or hydroxyC₁₋₄alkyl; and
each Z₁, R′, and R₄ is independently selected from halogen, hydroxyl, cyano, C₁₋₄alkyl, deutero-C₁₋₄alkyl, halo-C₁₋₄alkyl, amino, C₁₋₄alkylamino, (C₁₋₄alkyl)₂amino, C₁₋₄alkoxy, halo-C₁₋₄alkoxy, heteroaryl, heterocyclyl, and phenyl,
wherein heteroaryl is a 5-6 membered monocyclic or 9-10 membered bicyclic ring system having 1, 2, 3, or 4 heteroatom ring members selected from N, O, and S,
wherein heterocyclyl is a saturated or partially unsaturated 3-6 membered monocyclic or 9-10 membered bicyclic ring system having 1, 2, or 3 heteroatom ring members selected from N, O, and S, and
wherein each instance of phenyl, heteroaryl or heterocyclyl is optionally substituted with 1, or 2 substituents each selected from R₅;
R₅ is independently selected from halogen, hydroxyl, cyano, nitro, C₁₋₄alkyl, deutero-C₁₋₄alkyl, halo-C₁₋₄alkyl, amino, C₁₋₄alkyl-amino, (C₁₋₄alkyl)₂-amino, amino-C₁₋₄alkyl, hydroxyC₁₋₄alkyl, C₁₋₄alkyl-carbonyl, C₁₋₄alkoxy, C₁₋₄alkylthio, halo-C₁₋₄alkoxy, and C₃₋₁₀cycloalkyl; and
n is 0, 1, 2, or 3;
wherein a form of the compound is selected from the group consisting of a pharmaceutically acceptable salt, hydrate, solvate, racemate, enantiomer, diastereomer, stereoisomer, tautomer, and isotope enriched form thereof.

8. The compound of claim 1 having the structure of Formulae VIIIa, VIIIb, VIIIc, VIIId, VIIIXe, or VIIIf: 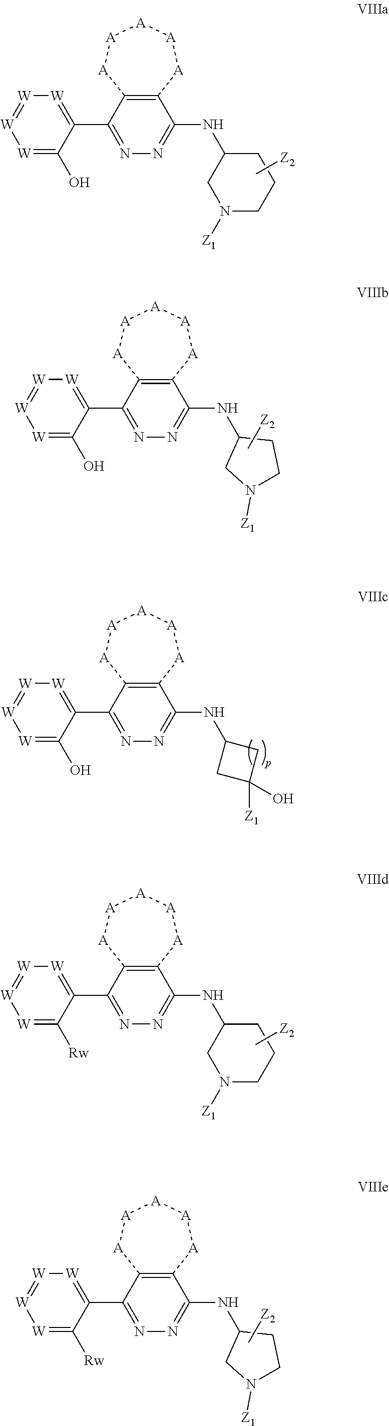 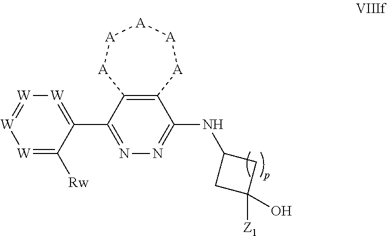
wherein
R_{w} is hydrogen, C₁₋₆alkyl, C₂₋₈alkynyl, halogen, C₁₋₆alkoxy, halo-C₁₋₄alkyl, halo-C₁₋₄alkoxy, cyano, —NH₂, —N(C₁₋₆alkyl)₂, thiol, —SO₂NH₂, —SO₂N(C₁₋₆alkyl)₂, —S(═O)(C₁₋₆alkyl), cycloalkyl, CHR₁ₐ, (C═O)R₁ₐ, OR₁ₐ, N(R_{1b})₂, S(═O)R₁ₐ, S(═O)₂R₁ₐ, O(C═O)R₁ₐ, (C═O)OR₁ₐ, NR_{1b}(C═O)R_{1b}, (C═O)NHR_{1b}, (C═O)N(R_{1b})₂ NR_{1b}(C═O)OR₁ₐ, O(C═O)N(R_{1b})₂, ONR_{1b}(C═NR_{1b})NR_{1b}, NR_{1b}S(═O)₂R₁ₐ or S(═O)₂N(R_{1b})₂, wherein each heterocycle, heteroaryl and aryl are optionally substituted with 1 or 2 substituents each selected from R₅;
each W is independently CH, CR′ or N;
each A is independently absent, CH, CH₂, CRₐ, CHRₐ, N, NH, NRₐ, S, or O;
each Rₐ is independently H, deuterium, halogen, —CN, —OH, —OR₂, ═O, ═N—OR₂, —SR₂, —S(═O)R₂, —S(═O)₂R², —N(R₂)₂, —NR₂S(═O)(═NR₂)R², —NR₂S(═O)₂R², —S(═O)₂N(R₂)₂, —C(═O)R₂, —OC(═O)R₂, —C(═O)OR₂, —OC(═O)OR₂, —C(═O)N(R₂)₂, —OC(═O)N(R₂)₂, —NR₂C(═O)R₂, —P(═O)(R₂)₂, C₁₋₄alkyl, (C₁₋₄alkyl)₂, halo-C₁₋₆alkyl, C₁₋₆heteroalkyl, C₃₋₈cycloalkyl, C₂₋₇heterocycloalkyl, aryl or monocyclic heteroaryl;
each -- represents either an absent, single, or double bond;
p is 0, 1, 2, 3 or 4;
each Z₁, Z₂, R₂, R′ or R₄ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, halo-C₁₋₄alkoxy, heteroaryl, heterocyclyl, and phenyl;
wherein heteroaryl is a 5-6 membered monocyclic or 9-10 membered bicyclic ring system having 1, 2, 3, or 4 heteroatom ring members selected from N, O, and S,
wherein heterocyclyl is a saturated or partially unsaturated 3-6 membered monocyclic or 9-10 membered bicyclic ring system having 1, 2, or 3 heteroatom ring members selected from N, O, and S, and
wherein each instance of phenyl, heteroaryl or heterocyclyl is optionally substituted with 1, or 2 substituents each selected from R₅;
R₅ is independently selected from halogen, hydroxyl, cyano, nitro, C₁₋₄alkyl, deutero-C₁₋₄alkyl, halo-C₁₋₄alkyl, amino, C₁₋₄alkylamino, (C₁₋₄alkyl)₂amino, aminoC₁₋₄alkyl, hydroxylC₁₋₄alkyl, C₁₋₄alkylcarbonyl, C₁₋₄alkoxy, C₁₋₄alkylthio, halo-C₁₋₄alkoxy, and C₃₋₁₀cycloalkyl
wherein a form of the compound is selected from the group consisting of a pharmaceutically acceptable salt, hydrate, solvate, racemate, enantiomer, diastereomer, stereoisomer, tautomer, and isotope enriched form thereof.

9. The compound of claim 2 having the structure of Formula IX 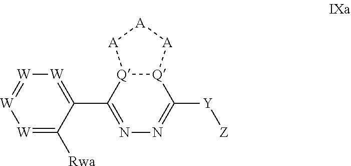
wherein:
each W is independently CH, CR′ or N;
each Q′ is independently N, or C, wherein at least one Q′ is N;
each A is independently CH, CH₂, CRₐ, CR₄, N, NH or NR₄;
Y is NR_{1b} or a bond;
Each R_{wa} is hydrogen, hydroxyl, C₁₋₄alkyl, halo-C₁₋₄alkyl, C₁₋₄alkoxy, or halo-C₁₋₄alkoxy,
each R_{1b} is independently hydrogen, C₁₋₄alkyl, deutero-C₁₋₄alkyl, halo-C₁₋₄alkyl or hydroxyC₁₋₄alkyl;
each Z is heterocyclyl, heteroaryl, aryl, C₃₋₁₀cycloalkyl, C₁₋₄alkyl, deutero-C₁₋₄alkyl, halo-C₁₋₄alkyl, hydroxy-C₁₋₈alkyl, NH(hydroxy-C₁₋₆alkyl), NH(C₁₋₆alkoxy) wherein each Z is optionally substituted with OH, NH₂, —CO₂H, halogen, C₁₋₆alkyl, C₁₋₆haloalkyl, C₁₋₆ hydroxyalkyl, C₂₋₆acyl, C₂₋₆alkanoic acid, C₂₋₆alkanoate ester, or heterocyclyl, and wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S, each optionally substituted with 1, 2, 3, 4, or 5 substituents each selected from R₂;
R₂ is independently selected from halogen, hydroxyl, cyano, C₁₋₄alkyl, deutero-C₁₋₄alkyl, halo-C₁₋₄alkyl, amino, C₁₋₄alkyl-amino, (C₁₋₆alkyl)₂-amino, halo-C₁₋₄alkyl-amino, (halo-C₁₋₆alkyl)₂-amino, hydroxy-C₁₋₄alkyl-amino, C₁₋₄alkoxy-C₁₋₄alkyl-amino, amino-C₁₋₄alkyl, C₁₋₄alkyl-amino-C₁₋₄alkyl, (C₁₋₄alkyl-amino)₂-C₁₋₄alkyl, C₁₋₄alkoxy, halo-C₁₋₄alkoxy, hydroxy-C₁₋₄alkoxy, C₁₋₄alkyl-C₁₋₄alkoxy, C₃₋₁₀cycloalkyl, C₃₋₁₀cycloalkyl-amino, C₃₋₁₀cycloalkyl-amino-C₁₋₄alkyl, heteroaryl-C₁₋₄alkyl, heteroaryl-amino, heteroaryl-C₁₋₄alkyl-amino, heterocyclyl, heterocyclyl-C₁₋₄alkyl, heterocyclyl-amino, heterocyclyl-amino-C₁₋₄alkyl, heterocyclyl-C₁₋₄alkoxy, heterocyclyl-amino-C₃₋₁₀cycloalkyl, phenyl, and phenyl-C₁₋₄alkoxy,
wherein heteroaryl is a 5-6 membered monocyclic or 6-10 membered bicyclic ring system having 1, 2, 3, or 4 heteroatom ring members independently selected from N, O, or S,
wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S,
wherein C₃₋₁₀cycloalkyl is a saturated or partially unsaturated 3-7 membered monocyclic ring system,
wherein each instance of phenyl, heteroaryl, heterocyclyl, or C₃₋₁₀cycloalkyl is optionally substituted with 1 or 2 substituents each selected from R₃;
R₃ is independently selected from halogen, hydroxyl, cyano, C₁₋₄alkyl, deutero-C₁₋₄alkyl, halo-C₁₋₄alkyl, amino, C₁₋₄alkoxy, and halo-C₁₋₄alkoxy;
R′ is selected from H, substituted or unsubstituted cycloalkyl, halo-C₁₋₄alkyl, haloalkoxy, halogen, C₁₋₆ alkoxy, cyano, hydroxyC₁₋₄alkyl or aryl optionally substituted by C₁₋₄alkyl;
each Rₐ is independently H, deuterium, halogen, —CN, —OH, —OR₂, ═O, ═N—OR₂, —SR₂, —S(═O)R₂, —S(═O)₂R², —N(R₂)₂, —NR₂S(═O)(═NR₂)R², —NR₂S(═O)₂R², —S(═O)₂N(R₂)₂, —C(═O)R₂, —OC(═O)R₂, —C(═O)OR₂, —OC(═O)OR₂, —C(═O)N(R₂)₂, —OC(═O)N(R₂)₂, —NR₂C(═O)R₂, —P(═O)(R₂)₂, C₁₋₄alkyl, (C₁₋₄alkyl)₂, halo-C₁₋₆alkyl, C₁₋₆heteroalkyl, C₃₋₈cycloalkyl, C₂₋₇heterocycloalkyl, aryl or monocyclic heteroaryl; and
R₄ is independently selected from halogen, hydroxyl, cyano, or C₁₋₄alkyl;
wherein a form of the compound is selected from the group consisting of a pharmaceutically acceptable salt, hydrate, solvate, racemate, enantiomer, diastereomer, stereoisomer, tautomer, and isotope enriched form thereof.

10. The compound of claim 9 having the structure of Formulae Xa, Xb, Xc, Xd, Xe or Xf: 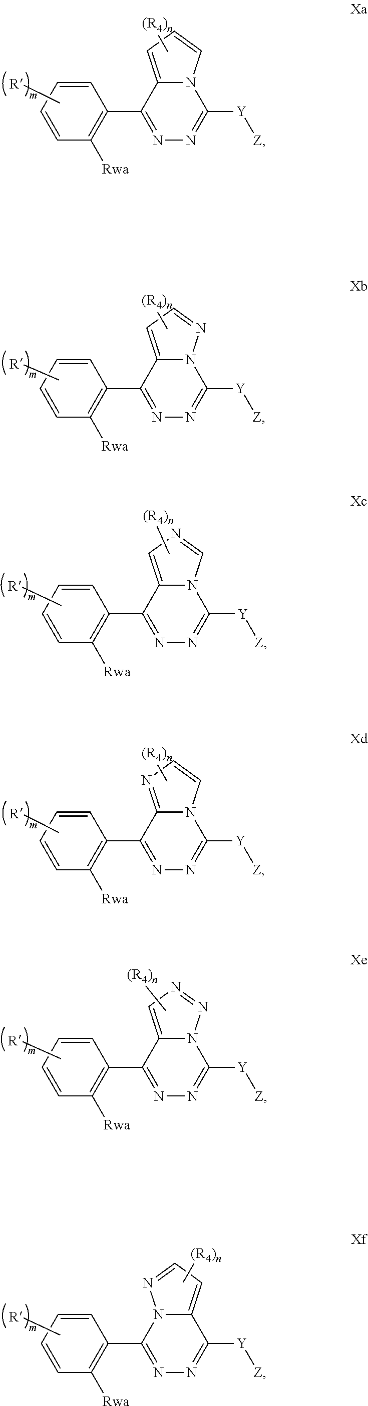
Wherein:
each R_{wa} is hydrogen, hydroxyl, C₁₋₄alkyl, halo-C₁₋₄alkyl, C₁₋₄alkoxy, or halo-C₁₋₄alkoxy
R′ is selected from hydrogen, halogen, hydroxyl, cyano, nitro, C₁₋₄alkyl, deutero-C₁₋₄alkyl, halo-C₁₋₄alkyl, amino, C₁₋₄alkyl-amino, (C₁₋₄alkyl)₂-amino, amino-C₁₋₄alkyl, hydroxyC₁₋₄alkyl, C₁₋₄alkyl-carbonyl, C₁₋₄alkoxy, C₁₋₄alkylthio, halo-C₁₋₄alkoxy, and C₃₋₁₀cycloalkyl; and
R₄ is selected from hydrogen, C₁₋₄ alkyl, halogen and halo-C₁₋₄alkyl;
Y is NR_{1b} or a bond;
each R_{1b} is independently hydrogen, C₁₋₄alkyl, deutero-C₁₋₄alkyl, halo-C₁₋₄alkyl or hydroxyC₁₋₄alkyl; and
each Z is heterocyclyl, heteroaryl, aryl, C₃₋₁₀cycloalkyl, C₁₋₄alkyl, deutero-C₁₋₄alkyl, halo-C₁₋₄alkyl, hydroxy-C₁₋₈alkyl, NH(hydroxy-C₁₋₆alkyl), NH(C₁₋₆alkoxy) wherein each Z is optionally substituted with OH, NH₂, —CO₂H, halogen, C₁₋₆alkyl, C₁₋₆haloalkyl, C₁₋₆ hydroxyalkyl, C₂₋₆acyl, C₂₋₆alkanoic acid, C₂₋₆alkanoate ester, or heterocyclyl, and wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S, each optionally substituted with 1, 2, 3, 4, or 5 substituents each selected from R₂;
R₂ is independently selected from halogen, hydroxy, cyano, C₁₋₄alkyl, hydroxy-C₁₄alkyl, deuterium-C₁₄alkyl, halo-C₁₄alkyl, amino, C₁₄alkylamino, (C₁₆alkyl)₂amino, halo-C₁₄alkylamino, (halo-C₁₆alkyl)₂amino, hydroxy-C₁₄alkylamino, C₁₄alkoxy-C₁₄alkyl-amino, aminoC₁₄alkyl, C₁₄alkylaminoC₁₄alkyl, (C₁₄alkylamino)₂C₁₄alkyl, C₁₄alkoxy, halo-C₁₄alkoxy, hydroxy-C₁₄alkoxy, C₁₄alkyl-C₁₄alkoxy, C₃₁₀cycloalkyl, C₃₁₀cycloalkyl-amino, C₃₁₀cycloalkyl-amino-C₁₄alkyl, heteroarylC₁₄alkyl, heteroarylamino, heteroarylC₁₄alkyl-amino, heterocyclyl, heterocyclyl-C₁₄alkyl, heterocyclyl-amino, heterocyclyl-amino-C₁₄alkyl, heterocyclyl-C₁₋₄alkoxy, heterocyclyl-amino-C₃₁₀cycloalkyl, phenyl, and phenyl-C₁₄alkoxy,
wherein heteroaryl is a 5-6 membered monocyclic or 6-10 membered bicyclic ring system having 1, 2, 3, or 4 heteroatom ring members independently selected from N, O, and S,
wherein heterocyclyl is a saturated or partially unsaturated 3-7 membered monocyclic, 6-10 membered bicyclic or 13-16 membered polycyclic ring system having 1, 2, or 3 heteroatom ring members independently selected from N, O, and S,
wherein C₃₋₁₀cycloalkyl is a saturated or partially unsaturated 3-7 membered monocyclic ring system, and
wherein each instance of phenyl, heteroaryl, heterocyclyl, or C₃₋₁₀cycloalkyl is optionally substituted with 1 or 2 substituents each selected from R₃; and
R₃ is independently selected from halogen, hydroxyl, cyano, C₁₋₄alkyl, deuterium-C₁₋₄alkyl, halo-C₁₋₄alkyl, amino, C₁₋₄alkoxy, and halo-C₁₋₄alkoxy;
m is 1, 2, 3, 4, or 5; and
n is 0, 1, 2, or 3;

11. The compound of claim 10 having the structure of Formulae XIa, XIb, XIc: 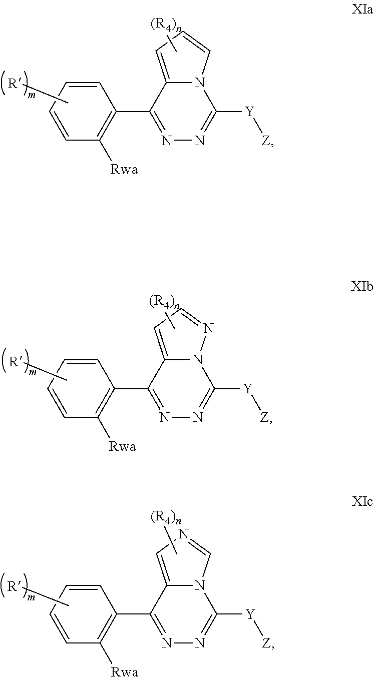
wherein the moiety having the structure 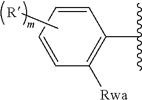
is selected from: 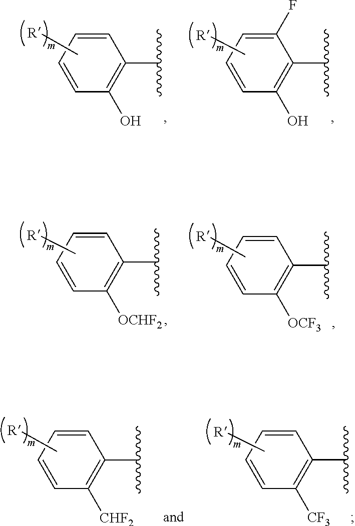
R′ is selected from halogen, hydroxy, cyano, nitro, C₁₋₄alkyl, deutero-C₁₋₄alkyl, halo-C₁₋₄alkyl, amino, C₁₋₄alkyl-amino, (C₁₋₄alkyl)₂-amino, amino-C₁₋₄alkyl, hydroxy-C₁₋₄alkyl, C₁₋₄alkyl-carbonyl, C₁₋₄alkoxy, C₁₋₄alkylthio, halo-C₁₋₄alkoxy, and C₃₋₁₀cycloalkyl;
Y and Z when taken together is selected from: 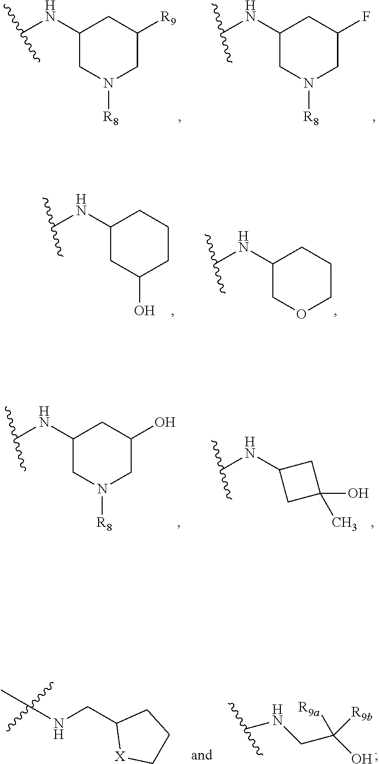
R₄ is selected from hydrogen, C₁₋₄ alkyl, halogen and halo-C₁₋₄alkyl;
R₈ is selected from C₁₋₄alkyl, CH₂CH₂OH, CH₂CH₂OCF₃, CH₂CH₂OCHF₂, and CH₂CH₂C(CH₃)₂OH;
R₉, R₉ₐ, R_{9b} is selected from hydrogen or C₁₋₄alkyl or C₃₋₆cycloalkyl; and C₉a and C₉b can optionally cyclized to form a 3-6 membered ring.
m is 1, 2, 3, 4, or 5.
n is 0, 1, 2, or 3.
X is selected from 0 or NR₈; and
wherein a form of the compound is selected from the group consisting of a pharmaceutically acceptable salt, hydrate, solvate, racemate, enantiomer, diastereomer, stereoisomer, tautomer, and isotope enriched form thereof.

12. The compound of claim 11, wherein the moiety having the structure: 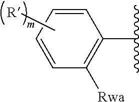
is selected from: 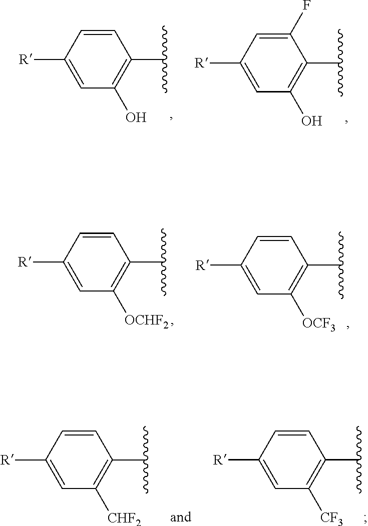
wherein a form of the compound is selected from the group consisting of a pharmaceutically acceptable salt, hydrate, solvate, racemate, enantiomer, diastereomer, stereoisomer, tautomer, and isotope enriched form thereof.

13. The compound of claim 12, wherein Y and Z when taken together is selected from: 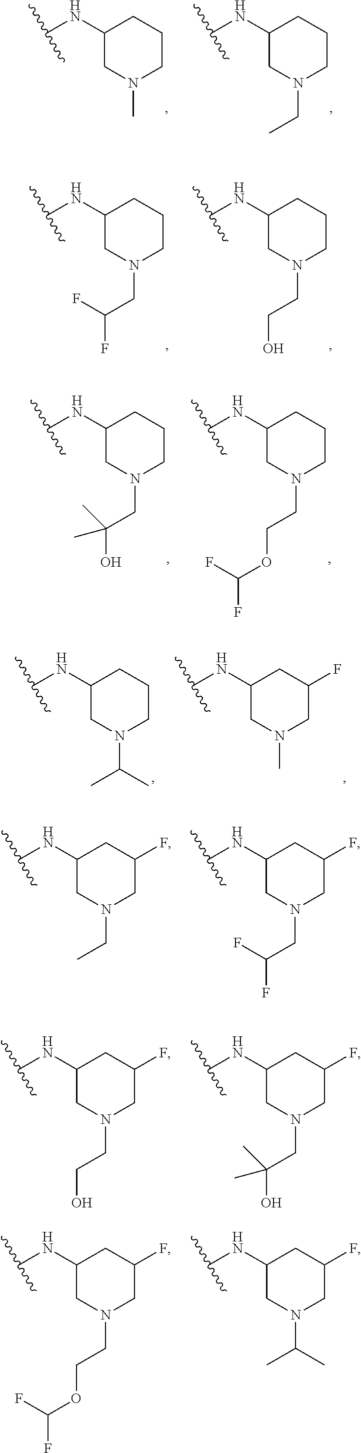 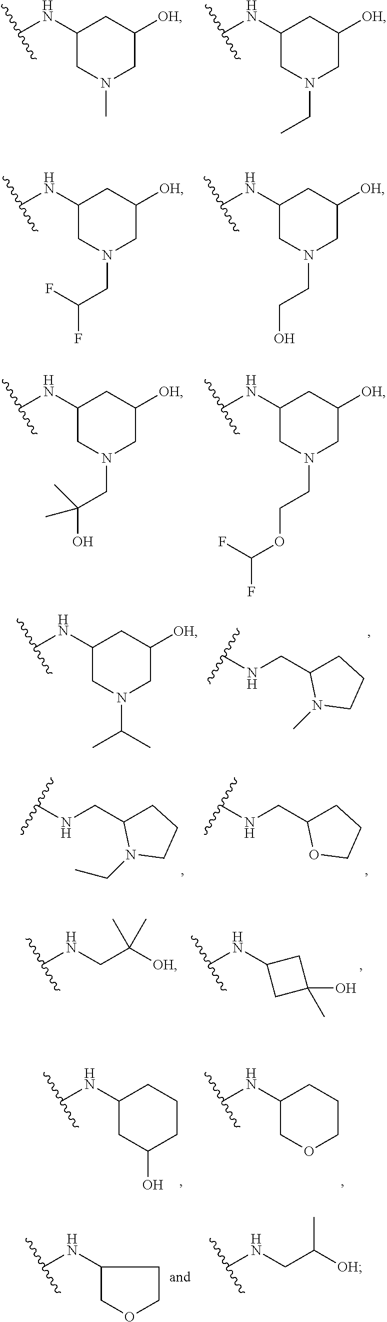
wherein a form of the compound is selected from the group consisting of a pharmaceutically acceptable salt, hydrate, solvate, racemate, enantiomer, diastereomer, stereoisomer, tautomer, and isotope enriched form thereof.

14. A compound or form thereof, selected from the group consisting of:
2-(4-{[(3R)-1-methylpiperidin-3-yl]amino}phthalazin-1-yl)-5-(trifluoromethyl)phenol;
2-(4-{[(3R)-1-methylpiperidin-3-yl]amino}-5,6,7,8-tetrahydrophthalazin-1-yl)-5-(trifluoromethyl)phenol;
2-(4-{[(3R)-1-methylpiperidin-3-yl]amino}-6,7-dihydro-5H-cyclopenta[d]pyridazin-1-yl)-5-(trifluoromethyl)phenol;
2-(4-{[(3R)-1-methylpiperidin-3-yl]amino}thieno[2,3-d]pyridazin-7-yl)-5-(trifluoromethyl)phenol;
2-(8-{[(3R)-1-methylpiperidin-3-yl]amino}pyrido[2,3-d]pyridazin-5-yl)-5-(trifluoromethyl)phenol;
2-{4-[(pyrrolidin-3-yl)amino]phthalazin-1-yl}-5-(trifluoromethyl)phenol;
2-(4-{[(3R)-1-methylpiperidin-3-yl]amino}pyrido[3,4-d]pyridazin-1-yl)-5-(trifluoromethyl)phenol;
2-(8-methyl-4-{[(3R)-1-methylpiperidin-3-yl]amino}phthalazin-1-yl)-5-(trifluoromethyl)phenol;
2-(7-fluoro-4-{[(3R)-1-methylpiperidin-3-yl]amino}phthalazin-1-yl)-5-(trifluoromethyl)phenol;
2-(4-{[(3R)-1-methylpiperidin-3-yl]amino}phthalazin-1-yl)-5-(2H-1,2,3-triazol-2-yl)phenol;
2-(4-{[(3R)-piperidin-3-yl]amino}-5,6,7,8-tetrahydrophthalazin-1-yl)-5-(trifluoromethyl)phenol;
2-(4-{[(3 S)-1-methylpiperidin-3-yl]amino}phthalazin-1-yl)-5-(trifluoromethyl)phenol;
5-chloro-2-(4-{[(3R)-1-methylpiperidin-3-yl]amino}phthalazin-1-yl)phenol;
3-fluoro-2-(4-{[(3R)-1-methylpiperidin-3-yl]amino}phthalazin-1-yl)phenol;
4-(4-{[(3R)-1-methylpiperidin-3-yl]amino}phthalazin-1-yl)benzene-1,3-diol;
5-fluoro-2-(4-{[(3R)-1-methylpiperidin-3-yl]amino}phthalazin-1-yl)phenol;
4-methyl-2-(4-{[(3R)-1-methylpiperidin-3-yl]amino}phthalazin-1-yl)phenol;
4-fluoro-5-methyl-2-(4-{[(3R)-1-methylpiperidin-3-yl]amino}phthalazin-1-yl)phenol;
2-(4-{[(3R)-1-ethylpiperidin-3-yl]amino}-5,6,7,8-tetrahydrophthalazin-1-yl)-5-(trifluoromethyl)phenol;
2-(4-{[(3R)-piperidin-3-yl]amino}phthalazin-1-yl)-5-(trifluoromethyl)phenol;
2-(4-{[(3R)-piperidin-3-yl]oxy}phthalazin-1-yl)-5-(trifluoromethyl)phenol;
2-(4-{[(3 S)-1-methylpiperidin-3-yl]oxy}phthalazin-1-yl)-5-(trifluoromethyl)phenol;
2-(8-methyl-4-{[(3R)-piperidin-3-yl]amino}-5,6,7,8-tetrahydrophthalazin-1-yl)-5-(trifluoromethyl)phenol;
3-hydroxy-4-(4-{[(3R)-1-methylpiperidin-3-yl]amino}phthalazin-1-yl)benzonitrile;
5-methyl-2-(4-{[(3R)-1-methylpiperidin-3-yl]amino}phthalazin-1-yl)phenol;
2-(4-{[(3R)-1-methylpyrrolidin-3-yl]amino}-5,6,7,8-tetrahydrophthalazin-1-yl)-5-(trifluoromethyl)phenol;
2-methyl-6-(4-{[(3R)-1-methylpiperidin-3-yl]amino}phthalazin-1-yl)phenol;
2-fluoro-6-(4-{[(3R)-1-methylpiperidin-3-yl]amino}phthalazin-1-yl)phenol;
2-(4-{[(3R)-1-methylpiperidin-3-yl]oxy}phthalazin-1-yl)-5-(trifluoromethyl)phenol;
2-{4-[(piperidin-3-yl)methyl]phthalazin-1-yl}-5-(trifluoromethyl)phenol;
2-(4-{[(3R)-piperidin-3-yl]amino}-6,7,8,9-tetrahydro-5H-cyclohepta[d]pyridazin-1-yl)-5-(trifluoromethyl)phenol;
5-(5-methyl-1,2,4-thiadiazol-3-yl)-2-(4-{[(3R)-piperidin-3-yl]amino}phthalazin-1-yl)phenol;
2-(4-{[(3R)-1-methylpiperidin-3-yl]amino}phthalazin-1-yl)phenol;
2-(4-{[(3R)-1-(propan-2-yl)piperidin-3-yl]amino}-5,6,7,8-tetrahydrophthalazin-1-yl)-5-(trifluoromethyl)phenol;
2-(4-{[(3R)-piperidin-3-yl]amino}pyrido[3,4-d]pyridazin-1-yl)-5-(trifluoromethyl)phenol;
2-(4-{[(3R)-1-methylpiperidin-3-yl]amino}-6,7,8,9-tetrahydro-5H-cyclohepta[d]pyridazin-1-yl)-5-(trifluoromethyl)phenol;
2-(4-{[(3R)-1-methylpiperidin-3-yl]amino}phthalazin-1-yl)-5-(1H-1,2,3-triazol-1-yl)phenol;
2-(4-{[(3R)-1-methylpiperidin-3-yl]amino}phthalazin-1-yl)-5-(5-methyl-1,2,4-thiadiazol-3-yl)phenol;
4-(4-{[(3R)-1-methylpiperidin-3-yl]amino}phthalazin-1-yl)[1,1′-biphenyl]-3-ol;
3-(4-{[(3R)-1-methylpiperidin-3-yl]amino}phthalazin-1-yl)[1,1′-biphenyl]-4-ol;
4,5-dimethyl-2-(4-{[(3R)-1-methylpiperidin-3-yl]amino}phthalazin-1-yl)phenol;
2,3-dimethyl-6-(4-{[(3R)-1-methylpiperidin-3-yl]amino}phthalazin-1-yl)phenol;
2-(4-{[(3R)-1-ethylpiperidin-3-yl]amino}pyrido[3,4-d]pyridazin-1-yl)-5-(trifluoromethyl)phenol;
2-{4-[(1-methylpiperidin-3-yl)methyl]phthalazin-1-yl}-5-(trifluoromethyl)phenol;
2-(7,7-dimethyl-4-{[(3R)-piperidin-3-yl]amino}-6,7-dihydro-5H-cyclopenta[d]pyridazin-1-yl)-5-(trifluoromethyl)phenol;
2-(7,7-dimethyl-4-{[(3R)-1-methylpiperidin-3-yl]amino}-6,7-dihydro-5H-cyclopenta[d]pyridazin-1-yl)-5-(trifluoromethyl)phenol;
2-(4-{methyl[(3R)-1-methylpiperidin-3-yl]amino}-5,6,7,8-tetrahydrophthalazin-1-yl)-5-(trifluoromethyl)phenol;
2-(4-{[(3R)-1-(2-hydroxyethyl)piperidin-3-yl]amino}phthalazin-1-yl)-5-(trifluoromethyl)phenol;
5-cyclopropyl-2-(4-{[(3R)-1-methylpiperidin-3-yl]amino}phthalazin-1-yl)phenol;
3-methyl-2-(4-{[(3R)-1-methylpiperidin-3-yl]amino}phthalazin-1-yl)phenol;
2-fluoro-3-methyl-6-(4-{[(3R)-1-methylpiperidin-3-yl]amino}phthalazin-1-yl)phenol;
5-(5,6-dihydro-2H-pyran-3-yl)-2-(4-{[(3R)-1-methylpiperidin-3-yl]amino}phthalazin-1-yl)phenol;
(3S,5R)-5-({4-[2-hydroxy-4-(trifluoromethyl)phenyl]phthalazin-1-yl}amino)piperidin-3-ol;
2-(1-{[(3R)-1-methylpiperidin-3-yl]amino}pyrido[3,4-d]pyridazin-4-yl)-5-(trifluoromethyl)phenol;
2-(4-{[(3R)-1-(2-hydroxyethyl)piperidin-3-yl]amino}pyrido[3,4-d]pyridazin-1-yl)-5-(trifluoromethyl)phenol;
5-(3,6-dihydro-2H-pyran-4-yl)-2-(4-{[(3R)-1-methylpiperidin-3-yl]amino}phthalazin-1-yl)phenol;
5-(2,5-dihydrofuran-3-yl)-2-(4-{[(3R)-1-methylpiperidin-3-yl]amino}phthalazin-1-yl)phenol;
5-(1-methyl-2,5-dihydro-1H-pyrrol-3-yl)-2-(4-{[(3R)-1-methylpiperidin-3-yl]amino}phthalazin-1-yl)phenol;
5-chloro-2-(1-{[(3R)-1-methylpiperidin-3-yl]amino}pyrido[3,4-d]pyridazin-4-yl)phenol;
5-chloro-2-(4-{[(3R)-1-methylpiperidin-3-yl]amino}pyrido[3,4-d]pyridazin-1-yl)phenol;
2-(4-{[(3R)-1-methylpiperidin-3-yl]amino}phthalazin-1-yl)-5-(oxolan-3-yl)phenol;
2-(4-{[(3R)-1-(2-hydroxyethyl)piperidin-3-yl]amino}-5,6,7,8-tetrahydrophthalazin-1-yl)-5-(trifluoromethyl)phenol;
(3S,5R)-5-({4-[2-hydroxy-4-(trifluoromethyl)phenyl]phthalazin-1-yl}amino)-1-methylpiperidin-3-ol;
1-[3-({4-[2-hydroxy-4-(trifluoromethyl)phenyl]phthalazin-1-yl}amino)piperidin-1-yl]ethan-1-one;
ethyl [(3R)-3-({4-[2-hydroxy-4-(trifluoromethyl)phenyl]phthalazin-1-yl}amino)piperidin-1-yl]acetate;
2-(5-{[(3R)-1-methylpiperidin-3-yl]amino}pyrido[2,3-d]pyridazin-8-yl)-5-(trifluoromethyl)phenol;
5-chloro-2-(8-{[(3R)-1-methylpiperidin-3-yl]amino}pyrido[2,3-d]pyridazin-5-yl)phenol;
2-[4-({[(2S)-pyrrolidin-2-yl]methyl}amino)phthalazin-1-yl]-5-(trifluoromethyl)phenol;
5-chloro-3-fluoro-2-(4-{[(3R)-1-methylpiperidin-3-yl]amino}phthalazin-1-yl)phenol;
5-methyl-2-(4-{[(3R)-1-methylpiperidin-3-yl]amino}pyrido[3,4-d]pyridazin-1-yl)phenol;
3-fluoro-2-(4-{[(3R)-1-methylpiperidin-3-yl]amino}pyrido[3,4-d]pyridazin-1-yl)phenol;
2-methoxy-6-(4-{[(3R)-1-methylpiperidin-3-yl]amino}pyrido[3,4-d]pyridazin-1-yl)phenol;
4-hydroxy-5-(4-{[(3R)-1-methylpiperidin-3-yl]amino}phthalazin-1-yl)pyridine-2-carbonitrile;
3-(4-{[(3R)-1-methylpiperidin-3-yl]amino}phthalazin-1-yl)-6-(trifluoromethyl)pyridin-2-01;
2-chloro-6-(4-{[(3R)-1-methylpiperidin-3-yl]amino}pyrido[3,4-d]pyridazin-1-yl)phenol;
2-cyclopropyl-6-(4-{[(3R)-1-methylpiperidin-3-yl]amino}pyrido[3,4-d]pyridazin-1-yl)phenol;
6-methyl-3-(4-{[(3R)-1-methylpiperidin-3-yl]amino}pyrido[3,4-d]pyridazin-1-yl)pyridin-2-ol;
5-(4-{[(3R)-1-methylpiperidin-3-yl]amino}phthalazin-1-yl)-2-(trifluoromethyl)pyridin-4-01;
6-hydroxy-5-(4-{[(3R)-1-methylpiperidin-3-yl]amino}phthalazin-1-yl)pyridine-2-carbonitrile;
5-chloro-2-(4-{[(3R)-1-methylpiperidin-3-yl]amino}phthalazin-1-yl)pyridin-3-ol;
2-methyl-6-(4-{[(3R)-1-methylpiperidin-3-yl]amino}pyrido[3,4-d]pyridazin-1-yl)phenol;
(3R,5R)-5-({4-[2-hydroxy-4-(trifluoromethyl)phenyl]phthalazin-1-yl}amino)piperidin-3-ol;
(3R,5R)-5-({4-[2-hydroxy-4-(trifluoromethyl)phenyl]phthalazin-1-yl}amino)-1-methylpiperidin-3-ol;
2-(4-{[(3R)-1-methylpiperidin-3-yl]amino}furo[2,3-d]pyridazin-7-yl)-5-(trifluoromethyl)phenol;
5-methyl-2-(8-{[(3R)-1-methylpiperidin-3-yl]amino}pyrido[2,3-d]pyridazin-5-yl)phenol;
2-[4-({[(2S)-1-methylpyrrolidin-2-yl]methyl}amino)phthalazin-1-yl]-5-(trifluoromethyl)phenol;
4-fluoro-5-methyl-2-(4-{[(3R)-1-methylpiperidin-3-yl]amino}pyrido[3,4-d]pyridazin-1-yl)phenol;
5-cyclopropyl-2-(4-{[(3R)-1-methylpiperidin-3-yl]amino}pyrido[3,4-d]pyridazin-1-yl)phenol;
5-methyl-2-(4-{[(3R)-1-methylpiperidin-3-yl]amino}phthalazin-1-yl)pyridin-3-ol;
5-cyclopropyl-2-(4-{[(3R)-1-methylpiperidin-3-yl]amino}phthalazin-1-yl)pyridin-3-ol;
3-hydroxy-4-(4-{[(3R)-1-methylpiperidin-3-yl]amino}pyrido[3,4-d]pyridazin-1-yl)benzonitrile;
2-(4-{[(3R)-1-methylpiperidin-3-yl]amino}phthalazin-1-yl)-5-(pyrrolidin-1-yl)phenol;
2-(4-{[(3R)-1-methylpiperidin-3-yl]amino}phthalazin-1-yl)-5-(1-methyl-1H-pyrrol-3-yl)phenol;
2-{4-[(1-methylazepan-3-yl)amino]phthalazin-1-yl}-5-(trifluoromethyl)phenol;
2-(4-{[(3 S)-1-methylpiperidin-3-yl]methyl}phthalazin-1-yl)-5-(trifluoromethyl)phenol;
2-(4-{[(3R)-1-methylpiperidin-3-yl]methyl}phthalazin-1-yl)-5-(trifluoromethyl)phenol;
[(3R)-3-({4-[2-hydroxy-4-(trifluoromethyl)phenyl]phthalazin-1-yl}amino)piperidin-1-yl]acetic acid;
5-methyl-2-(1-{[(3R)-1-methylpiperidin-3-yl]amino}pyrido[3,4-d]pyridazin-4-yl)phenol;
2-{1-[(piperidin-3-yl)methyl]pyrido[3,4-d]pyridazin-4-yl}-5-(trifluoromethyl)phenol;
2-(1-{[(3R)-1-(2-hydroxyethyl)piperidin-3-yl]amino}pyrido[3,4-d]pyridazin-4-yl)-5-(trifluoromethyl)phenol;
2-(4-{[(3R)-1-methylpiperidin-3-yl]amino}pyrido[3,4-d]pyridazin-1-yl)-5-(trifluoromethoxy)phenol;
3-methyl-2-(4-{[(3R)-1-methylpiperidin-3-yl]amino}pyrido[3,4-d]pyridazin-1-yl)-5-(trifluoromethyl)phenol;
2-(8-chloro-4-{[(3R)-1-methylpiperidin-3-yl]amino}pyrrolo[1,2-d][1,2,4]triazin-1-yl)-5-(trifluoromethyl)phenol;
3-methyl-2-(4-{[(3R)-1-methylpiperidin-3-yl]amino}pyrido[3,4-d]pyridazin-1-yl)phenol;
2-(4-{[(3R)-1-methylpiperidin-3-yl]amino}pyrrolo[1,2-d][1,2,4]triazin-1-yl)-5-(trifluoromethyl)phenol;
2-(4-{[(3R)-1-methylpiperidin-3-yl]amino}phthalazin-1-yl)-5-(1-methylpyrrolidin-3-yl)phenol;
2-(4-{[2-(morpholin-4-yl)ethyl]amino}phthalazin-1-yl)-5-(trifluoromethyl)phenol;
2-{4-[(azetidin-3-yl)amino]phthalazin-1-yl}-5-(trifluoromethyl)phenol;
2-[1-({[(2S)-pyrrolidin-2-yl]methyl}amino)pyrido[3,4-d]pyridazin-4-yl]-5-(trifluoromethyl)phenol;
2-[4-({[(2S)-pyrrolidin-2-yl]methyl}amino)pyrido[3,4-d]pyridazin-1-yl]-5-(trifluoromethyl)phenol;
2-[4-({[(2S)-1-methylpyrrolidin-2-yl]methyl}amino)pyrido[3,4-d]pyridazin-1-yl]-5-(trifluoromethyl)phenol;
2-[4-({[(2S)-1-ethylpyrrolidin-2-yl]methyl}amino)pyrido[3,4-d]pyridazin-1-yl]-5-(trifluoromethyl)phenol;
2-(4-{[2-(piperidin-1-yl)ethyl]amino}phthalazin-1-yl)-5-(trifluoromethyl)phenol;
2-[1-(4-methylpiperazin-1-yl)pyrido[3,4-d]pyridazin-4-yl]-5-(trifluoromethyl)phenol;
2-(4-methyl-5-{[(3R)-1-methylpiperidin-3-yl]amino}-3,4-dihydro-2H-pyridazino[4,5-b][1,4]oxazin-8-yl)-5-(trifluoromethyl)phenol;
2-{1-[(1-methylpiperidin-3-yl)methyl]pyrido[3,4-d]pyridazin-4-yl}-5-(trifluoromethyl)phenol;
1-[2-methoxy-4-(trifluoromethyl)phenyl]-N-[(3R)-1-methylpiperidin-3-yl]pyrido[3,4-d]pyridazin-4-amine;
4-[2-(difluoromethoxy)-4-(trifluoromethyl)phenyl]-N-[(3R)-1-methylpiperidin-3-yl]phthalazin-1-amine;
1-[2-(difluoromethoxy)-4-(trifluoromethyl)phenyl]-N-[(3R)-1-methylpiperidin-3-yl]pyrido[3,4-d]pyridazin-4-amine;
4-[2-(difluoromethoxy)-4-(trifluoromethyl)phenyl]-N-[(3R)-1-methylpiperidin-3-yl]pyrido[3,4-d]pyridazin-1-amine;
4-[2-amino-4-(trifluoromethyl)phenyl]-N-[(3R)-1-methylpiperidin-3-yl]phthalazin-1-amine;
N-[2-(4-{[(3R)-1-methylpiperidin-3-yl]amino}phthalazin-1-yl)-5-(trifluoromethyl)phenyl]methanesulfonamide;
4-[2-(methylamino)-4-(trifluoromethyl)phenyl]-N-[(3R)-1-methylpiperidin-3-yl]phthalazin-1-amine;
N-[2-(4-{[(3R)-1-methylpiperidin-3-yl]amino}phthalazin-1-yl)-5-(trifluoromethyl)phenyl]acetamide;
4-[2-fluoro-4-(trifluoromethyl)phenyl]-N-[(3R)-1-methylpiperidin-3-yl]phthalazin-1-amine;
1-[2-fluoro-4-(trifluoromethyl)phenyl]-N-[(3R)-1-methylpiperidin-3-yl]pyrido[3,4-d]pyridazin-4-amine;
1-(4-chloro-2-fluorophenyl)-N-[(3R)-1-methylpiperidin-3-yl]pyrido[3,4-d]pyridazin-4-amine;
2-(4-{[(3R)-1-methylpiperidin-3-yl]amino}pyrido[3,4-d]pyridazin-1-yl)-5-(trifluoromethyl)benzonitrile;
2-(4-{[(3R)-1-methylpiperidin-3-yl]amino}phthalazin-1-yl)-5-(trifluoromethyl)benzonitrile;
1-[4-chloro-2-(difluoromethoxy)phenyl]-N-[(3R)-1-methylpiperidin-3-yl]pyrido[3,4-d]pyridazin-4-amine;
4-[4-chloro-2-(difluoromethoxy)phenyl]-N-[(3R)-1-methylpiperidin-3-yl]phthalazin-1-amine;
1-[2-(difluoromethoxy)-4-methylphenyl]-N-[(3R)-1-methylpiperidin-3-yl]pyrido[3,4-d]pyridazin-4-amine;
4-[2-(difluoromethoxy)-4-methylphenyl]-N-[(3R)-1-methylpiperidin-3-yl]phthalazin-1-amine;
5-methyl-2-(4-{[(3R)-1-methylpiperidin-3-yl]amino}pyrido[3,4-d]pyridazin-1-yl)benzonitrile;
2-(4-{[(3R)-1-methylpiperidin-3-yl]amino}pyrido[3,4-d]pyridazin-1-yl)-5-(2H-1,2,3-triazol-2-yl)phenol;
1-[2-(difluoromethyl)-4-(trifluoromethyl)phenyl]-N-[(3R)-1-methylpiperidin-3-yl]pyrido[3,4-d]pyridazin-4-amine;
3-chloro-2-(4-{[(3S)-1-methylpiperidin-3-yl]amino}pyrido[3,4-d]pyridazin-1-yl)phenol;
3-hydroxy-2-(4-{[(3R)-1-methylpiperidin-3-yl]amino}pyrido[3,4-d]pyridazin-1-yl)benzonitrile;
3,5-dimethyl-2-(4-{[(3R)-1-methylpiperidin-3-yl]amino}pyrido[3,4-d]pyridazin-1-yl)phenol;
5-chloro-3-methyl-2-(4-{[(3R)-1-methylpiperidin-3-yl]amino}pyrido[3,4-d]pyridazin-1-yl)phenol;
1-[2-chloro-4-(trifluoromethyl)phenyl]-N-[(3R)-1-methylpiperidin-3-yl]pyrido[3,4-d]pyridazin-4-amine;
4-[2-methoxy-4-(trifluoromethyl)phenyl]-1-methyl-N-[(3R)-1-methylpiperidin-3-yl]-1H-pyrazolo[3,4-d]pyridazin-7-amine;
1-[2-(2,5-dihydrofuran-3-yl)-4-(trifluoromethyl)phenyl]-N-[(3R)-1-methylpiperidin-3-yl]pyrido[3,4-d]pyridazin-4-amine;
2-(1-methyl-7-{[(3R)-1-methylpiperidin-3-yl]amino}-1H-pyrazolo[3,4-d]pyridazin-4-yl)-5-(trifluoromethyl)phenol;
2-[4-({2-[(3R)-3-methylmorpholin-4-yl]ethyl}amino)phthalazin-1-yl]-5-(trifluoromethyl)phenol;
2-[4-({2-[(3 S)-3-methylmorpholin-4-yl]ethyl}amino)phthalazin-1-yl]-5-(trifluoromethyl)phenol;
2-(4-{[2-(piperazin-1-yl)ethyl]amino}phthalazin-1-yl)-5-(trifluoromethyl)phenol;
2-(4-{[(azetidin-2-yl)methyl]amino}phthalazin-1-yl)-5-(trifluoromethyl)phenol;
2-(4-{[(3R,5R)-5-fluoro-1-methylpiperidin-3-yl]amino}phthalazin-1-yl)-5-(trifluoromethyl)phenol;
2-{1-[3-(dimethylamino)piperidin-1-yl]pyrido[3,4-d]pyridazin-4-yl}-5-(trifluoromethyl)phenol;
4-[2-methoxy-4-(trifluoromethyl)phenyl]-1-(piperazin-1-yl)pyrido[3,4-d]pyridazine;
2-(1-methyl-8-{[(3R)-1-methylpiperidin-3-yl]amino}-1,2,3,4-tetrahydropyrido[2,3-d]pyridazin-5-yl)-5-(trifluoromethyl)phenol;
5-chloro-2-(1-methyl-8-{[(3S)-1-methylpiperidin-3-yl]amino}-1,2,3,4-tetrahydropyrido[2,3-d]pyridazin-5-yl)phenol;
2-(4-{[(3R)-1-methylpiperidin-3-yl]amino}pyrido[3,4-d]pyridazin-1-yl)-5-(trifluoromethyl)benzene-1-sulfonamide;
5-chloro-2-(4-{[(3R)-1-methylpiperidin-3-yl]amino}pyrido[3,4-d]pyridazin-1-yl)benzonitrile;
1-[4-cyclopropyl-2-(trifluoromethyl)phenyl]-N-[(3R)-1-methylpiperidin-3-yl]pyrido[3,4-d]pyridazin-4-amine;
3-methyl-5-(4-{[(3R)-1-methylpiperidin-3-yl]amino}phthalazin-1-yl)pyridin-4-ol;
4-(2-methoxy-6-methylpyridin-3-yl)-N-[(3R)-1-methylpiperidin-3-yl]phthalazin-1-amine;
2-(4-{[2-(4-methylpiperazin-1-yl)ethyl]amino}phthalazin-1-yl)-5-(trifluoromethyl)phenol;
1-{4-[2-methoxy-4-(trifluoromethyl)phenyl]pyrido[3,4-d]pyridazin-1-yl}-N,N-dimethylpiperidin-3-amine;
2-amino-3-chloro-6-(4-{[(3R)-1-methylpiperidin-3-yl]amino}phthalazin-1-yl)phenol;
2-(4-{[(3 S,5S)-5-fluoro-1-methylpiperidin-3-yl]amino}phthalazin-1-yl)-5-(trifluoromethyl)phenol;
4-(2,3-difluoro-4-methylphenyl)-N-[(3R)-1-methylpiperidin-3-yl]phthalazin-1-amine;
N-[(3R)-1-methylpiperidin-3-yl]-1-[4-(trifluoromethyl)phenyl]pyrido[3,4-d]pyridazin-4-amine;
3-(difluoromethoxy)-4-(4-{[(3R)-1-methylpiperidin-3-yl]amino}pyrido[3,4-d]pyridazin-1-yl)benzonitrile;
1-[2-(difluoromethoxy)-3-methylphenyl]-N-[(3R)-1-methylpiperidin-3-yl]pyrido[3,4-d]pyridazin-4-amine;
N-methyl-2-(4-{[(3R)-1-methylpiperidin-3-yl]amino}pyrido[3,4-d]pyridazin-1-yl)-5-(trifluoromethyl)benzene-1-sulfonamide;
5-chloro-3-fluoro-2-(4-{[(3R)-1-methylpiperidin-3-yl]amino}pyrido[3,4-d]pyridazin-1-yl)phenol;
1-[2-(difluoromethoxy)-4-methoxyphenyl]-N-[(3R)-1-methylpiperidin-3-yl]pyrido[3,4-d]pyridazin-4-amine;
5-chloro-2-(4-{[(3R)-1-methylpiperidin-3-yl]amino}pyrido[3,4-d]pyridazin-1-yl)pyridin-3-ol;
1-[2-(methoxymethoxy)-4-(1,3-oxazol-2-yl)phenyl]-N-[(3S)-1-methylpiperidin-3-yl]pyrido[3,4-d]pyridazin-4-amine;
2-(4-{[(1-methyl-1H-pyrazol-4-yl)methyl]amino}pyrido[3,4-d]pyridazin-1-yl)-5-(trifluoromethyl)phenol;
5-methoxy-2-(4-{[(3R)-1-methylpiperidin-3-yl]amino}pyrido[3,4-d]pyridazin-1-yl)phenol;
2-(4-{[(3 S)-1-methylpiperidin-3-yl]amino}pyrido[3,4-d]pyridazin-1-yl)-5-(1,3-oxazol-2-yl)phenol;
1-[2,4-bis(trifluoromethyl)phenyl]-N-[(3R)-1-methylpiperidin-3-yl]pyrido[3,4-d]pyridazin-4-amine;
1-[2-(difluoromethoxy)-5-fluoro-4-methylphenyl]-N-[(3R)-1-methylpiperidin-3-yl]pyrido[3,4-d]pyridazin-4-amine;
3-fluoro-5-methyl-2-(4-{[(3R)-1-methylpiperidin-3-yl]amino}pyrido[3,4-d]pyridazin-1-yl)phenol;
2-{4-[(1-methyl-1H-pyrazol-4-yl)amino]pyrido[3,4-d]pyridazin-1-yl}-5-(trifluoromethyl)phenol;
1-[4-methoxy-2-(trifluoromethyl)phenyl]-N-[(3R)-1-methylpiperidin-3-yl]pyrido[3,4-d]pyridazin-4-amine;
2-(4-{[(3R)-1-methylpiperidin-3-yl]amino}pyrido[3,4-d]pyridazin-1-yl)-5-(trifluoromethyl)benzamide;
5-[2-(difluoromethoxy)-4-(trifluoromethyl)phenyl]-N-[(3R)-1-methylpiperidin-3-yl]pyrido[2,3-d]pyridazin-8-amine;
8-[2-(difluoromethoxy)-4-(trifluoromethyl)phenyl]-N-[(3R)-1-methylpiperidin-3-yl]pyrido[2,3-d]pyridazin-5-amine;
2-{1-[(1-methylazepan-3-yl)amino]pyrido[3,4-d]pyridazin-4-yl}-5-(trifluoromethyl)phenol;
2-(4-{[(3 S)-1-methylazepan-3-yl]amino}pyrido[3,4-d]pyridazin-1-yl)-5-(trifluoromethyl)phenol;
2-(4-{[(3R)-1-methylazepan-3-yl]amino}pyrido[3,4-d]pyridazin-1-yl)-5-(trifluoromethyl)phenol;
N-[(3R)-1-methylpiperidin-3-yl]-1-{2-[(oxetan-3-yl)oxy]-4-(trifluoromethyl)phenyl}pyrido[3,4-d]pyridazin-4-amine;
N-[(3R)-1-methylpiperidin-3-yl]-1-[2-(1-methyl-1H-pyrazol-4-yl)-4-(trifluoromethyl)phenyl]pyrido[3,4-d]pyridazin-4-amine;
[2-(4-{[(3R)-1-methylpiperidin-3-yl]amino}pyrido[3,4-d]pyridazin-1-yl)-5-(trifluoromethyl)phenyl]methanol;
1-[2-(difluoromethoxy)-4-(trifluoromethoxy)phenyl]-N-[(3R)-1-methylpiperidin-3-yl]pyrido[3,4-d]pyridazin-4-amine;
1-[2-(methanesulfonyl)-4-(trifluoromethyl)phenyl]-N-[(3R)-1-methylpiperidin-3-yl]pyrido[3,4-d]pyridazin-4-amine;
N-[(3R)-1-methylpiperidin-3-yl]-1-[2-(1H-pyrazol-4-yl)-4-(trifluoromethyl)phenyl]pyrido[3,4-d]pyridazin-4-amine;
1-[2-cyclopropyl-4-(trifluoromethyl)phenyl]-N-[(3R)-1-methylpiperidin-3-yl]pyrido[3,4-d]pyridazin-4-amine;
5-methyl-2-(4-{[(3R)-1-methylpiperidin-3-yl]amino}pyrido[3,4-d]pyridazin-1-yl)pyridin-3-ol;
5-cyclopropyl-3-fluoro-2-(4-{[(3R)-1-methylpiperidin-3-yl]amino}pyrido[3,4-d]pyridazin-1-yl)phenol;
3-fluoro-2-(4-{[(3R)-1-methylpiperidin-3-yl]amino}pyrido[3,4-d]pyridazin-1-yl)-5-(trifluoromethyl)phenol;
1-(4-chloro-2-fluoro-6-methylphenyl)-N-[(3R)-1-methylpiperidin-3-yl]pyrido[3,4-d]pyridazin-4-amine;
N-[(3R)-1-azabicyclo[2.2.2]octan-3-yl]-1-[2-methoxy-4-(trifluoromethyl)phenyl]pyrido[3,4-d]pyridazin-4-amine;
2-(4-{[(3R)-1-methylpiperidin-3-yl]amino}pyrido[3,4-d]pyridazin-1-yl)-5-(pyrimidin-2-yl)phenol;
1-{4-[2-methoxy-4-(trifluoromethyl)phenyl]phthalazin-1-yl}-N,N-dimethylpiperidin-4-amine;
N-[(3R)-1-methylpiperidin-3-yl]-1-[5-(trifluoromethyl)[1,1′-biphenyl]-2-yl]pyrido[3,4-d]pyridazin-4-amine;
2-(4-{3-[(dimethylamino)methyl]pyrrolidin-1-yl}phthalazin-1-yl)-5-(trifluoromethyl)phenol;
1-[2-(difluoromethoxy)-6-(trifluoromethyl)pyridin-3-yl]-N-[(3R)-1-methylpiperidin-3-yl]pyrido[3,4-d]pyridazin-4-amine;
N-[(3R)-1-methylpiperidin-3-yl]-1-(2,4,6-trimethylphenyl)pyrido[3,4-d]pyridazin-4-amine;
2-[(3R)-3-({1-[2-(difluoromethoxy)-4-(trifluoromethyl)phenyl]pyrido[3,4-d]pyridazin-4-yl}amino)piperidin-1-yl]ethan-1-ol;
1-[2,4-di(propan-2-yl)phenyl]-N-[(3R)-1-methylpiperidin-3-yl]pyrido[3,4-d]pyridazin-4-amine;
1-(3-cyclopropyl-6-fluoro-2-methoxyphenyl)-N-[(3R)-1-methylpiperidin-3-yl]pyrido[3,4-d]pyridazin-4-amine;
6-chloro-3-fluoro-2-(4-{[(3R)-1-methylpiperidin-3-yl]amino}pyrido[3,4-d]pyridazin-1-yl)phenol;
6-cyclopropyl-3-fluoro-2-(4-{[(3R)-1-methylpiperidin-3-yl]amino}pyrido[3,4-d]pyridazin-1-yl)phenol;
6-chloro-3-fluoro-2-(5-{[(3R)-1-methylpiperidin-3-yl]amino}pyrido[2,3-d]pyridazin-8-yl)phenol;
2-{4-[(4-methyl-4-azaspiro[2.5]octan-7-yl)amino]pyrido[3,4-d]pyridazin-1-yl}-5-(trifluoromethyl)phenol;
1-[2-methoxy-4-(trifluoromethyl)phenyl]-N-(8-methyl-8-azabicyclo[3.2.1]octan-3-yl)pyrido[3,4-d]pyridazin-4-amine;
N-[(3R)-1-methylpiperidin-3-yl]-1-[2-(2,2,2-trifluoroethyl)-4-(trifluoromethyl)phenyl]pyrido[3,4-d]pyridazin-4-amine;
2-(4-{[(3R)-1-cyclobutylpiperidin-3-yl]amino}pyrido[3,4-d]pyridazin-1-yl)-5-(trifluoromethyl)phenol;
1-(2,4-dimethylphenyl)-N-[(3R)-1-methylpiperidin-3-yl]pyrido[3,4-d]pyridazin-4-amine;
3-methyl-4-(4-{[(3R)-1-methylpiperidin-3-yl]amino}pyrido[3,4-d]pyridazin-1-yl)benzonitrile;
1-(4-chloro-2,6-dimethylphenyl)-N-[(3R)-1-methylpiperidin-3-yl]pyrido[3,4-d]pyridazin-4-amine;
2-(4-{[(3R)-1-cyclopropylpiperidin-3-yl]amino}pyrido[3,4-d]pyridazin-1-yl)-5-(trifluoromethyl)phenol;
2,2,2-trifluoro-1-[3-hydroxy-4-(4-{[(3R)-piperidin-3-yl]amino}phthalazin-1-yl)phenyl]ethan-1-one;
1-(4-chloro-2,6-difluorophenyl)-N-[(3R)-1-methylpiperidin-3-yl]pyrido[3,4-d]pyridazin-4-amine;
1-[2-fluoro-6-methyl-4-(trifluoromethyl)phenyl]-N-[(3R)-1-methylpiperidin-3-yl]pyrido[3,4-d]pyridazin-4-amine;
2-(4-{[(3R)-1-methylpiperidin-3-yl]amino}pyrido[3,4-d]pyridazin-1-yl)-5-nitrophenol;
2-{4-[(1-methylpiperidin-4-yl)amino]pyrido[3,4-d]pyridazin-1-yl}-5-(trifluoromethyl)phenol;
5-(cyclopropylethynyl)-2-(4-{[(3R)-1-methylpiperidin-3-yl]amino}phthalazin-1-yl)phenol;
5-ethynyl-2-(4-{[(3R)-1-methylpiperidin-3-yl]amino}phthalazin-1-yl)phenol;
2-(4-{[(3R)-1-methylpiperidin-3-yl]amino}phthalazin-1-yl)-5-(prop-1-yn-1-yl)phenol;
N-[(3R)-1-methylpiperidin-3-yl]-1-[2-(prop-1-yn-1-yl)-4-(trifluoromethyl)phenyl]pyrido[3,4-d]pyridazin-4-amine;
2-{1-[(5,6,7,8-tetrahydroimidazo[1,2-a]pyridin-6-yl)amino]pyrido[3,4-d]pyridazin-4-yl}-5-(trifluoromethyl)phenol;
2-{1-[(1,4-oxazepan-6-yl)amino]pyrido[3,4-d]pyridazin-4-yl}-5-(trifluoromethyl)phenol;
4-[2-(difluoromethoxy)-4-(trifluoromethyl)phenyl]-N-[(3R)-piperidin-3-yl]pyrido[3,4-d]pyridazin-1-amine;
N-[(3R)-1-methylpiperidin-3-yl]-1-[2-(1-methyl-1H-pyrazol-3-yl)-4-(trifluoromethyl)phenyl]pyrido[3,4-d]pyridazin-4-amine;
2-{4-[3-(dimethylamino)piperidin-1-yl]pyrido[3,4-d]pyridazin-1-yl}-5-(trifluoromethyl)phenol;
1-[4-(difluoromethyl)-2-methoxyphenyl]-N-[(3R)-1-methylpiperidin-3-yl]pyrido[3,4-d]pyridazin-4-amine;
4-[2-(difluoromethoxy)-4-(trifluoromethyl)phenyl]-N-[(3R)-1-ethylpiperidin-3-yl]pyrido[3,4-d]pyridazin-1-amine;
4-[2-(difluoromethoxy)-4-(trifluoromethyl)phenyl]-N-[(3R)-1-methylpiperidin-3-yl]-6,7-dihydro-5H-cyclopenta[d]pyridazin-1-amine;
5-(cyclopropyloxy)-2-(4-{[(3R)-1-methylpiperidin-3-yl]amino}pyrido[3,4-d]pyridazin-1-yl)phenol;
2-[(3R)-3-({4-[2-(difluoromethoxy)-4-(trifluoromethyl)phenyl]pyrido[3,4-d]pyridazin-1-yl}amino)piperidin-1-yl]ethan-1-ol;
1-[4-chloro-2-(difluoromethyl)phenyl]-N-[(3R)-1-methylpiperidin-3-yl]pyrido[3,4-d]pyridazin-4-amine;
N-[(3R)-1-methylpiperidin-3-yl]-1-[2-(pyridin-3-yl)-4-(trifluoromethyl)phenyl]pyrido[3,4-d]pyridazin-4-amine;
4-[4-chloro-2-(difluoromethoxy)phenyl]-N-[(3R)-1-methylpiperidin-3-yl]-6,7-dihydro-5H-cyclopenta[d]pyridazin-1-amine;
1-(2-cyclopropyl-4-methoxyphenyl)-N-[(3R)-1-methylpiperidin-3-yl]pyrido[3,4-d]pyridazin-4-amine;
2-(4-{[(1-methylpiperidin-2-yl)methyl]amino}pyrido[3,4-d]pyridazin-1-yl)-5-(trifluoromethyl)phenol;
4-[2-(difluoromethoxy)-4-methylphenyl]-N-[(3R)-1-methylpiperidin-3-yl]-6,7-dihydro-5H-cyclopenta[d]pyridazin-1-amine;
4-[2-(difluoromethoxy)phenyl]-N-[(3R)-1-methylpiperidin-3-yl]-6,7-dihydro-5H-cyclopenta[d]pyridazin-1-amine;
4-[4-cyclopropyl-2-(difluoromethoxy)phenyl]-N-[(3R)-1-methylpiperidin-3-yl]-6,7-dihydro-5H-cyclopenta[d]pyridazin-1-amine;
1-[4-cyclopropyl-2-(difluoromethoxy)phenyl]-N-[(3R)-1-methylpiperidin-3-yl]pyrido[3,4-d]pyridazin-4-amine;
1-[4-cyclopropyl-2-(difluoromethyl)phenyl]-N-[(3R)-1-methylpiperidin-3-yl]pyrido[3,4-d]pyridazin-4-amine;
1-[4-(difluoromethyl)-2-fluorophenyl]-N-[(3R)-1-methylpiperidin-3-yl]pyrido[3,4-d]pyridazin-4-amine;
2-(4-{[(3R)-1-methylpiperidin-3-yl]amino}thieno[3,4-d]pyridazin-1-yl)-5-(trifluoromethyl)phenol;
N-[(3R)-azepan-3-yl]-1-[2-methoxy-4-(trifluoromethyl)phenyl]pyrido[3,4-d]pyridazin-4-amine;
2-{4-[(1,4-oxazepan-6-yl)amino]pyrido[3,4-d]pyridazin-1-yl}-5-(trifluoromethyl)phenol;
2-{4-[(azepan-4-yl)amino]pyrido[3,4-d]pyridazin-1-yl}-5-(trifluoromethyl)phenol;
1-(4-methoxy-2-methylphenyl)-N-[(3R)-1-methylpiperidin-3-yl]pyrido[3,4-d]pyridazin-4-amine;
2-(3-methyl-4-{[(3R)-1-methylpiperidin-3-yl]amino}[1,2]oxazolo[4,5-d]pyridazin-7-yl)-5-(trifluoromethyl)phenol;
2-(4-{[(3R)-1-(2-hydroxyethyl)piperidin-3-yl]amino}-3-methyl[1,2]oxazolo[4,5-d]pyridazin-7-yl)-5-(trifluoromethyl)phenol;
2-[4-({(3R)-1-[3-(2,2-difluoroethyl)cyclobutyl]piperidin-3-yl}amino)pyrido[3,4-d]pyridazin-1-yl]-5-(trifluoromethyl)phenol;
4-[4-methoxy-2-(trifluoromethyl)phenyl]-N-[(3R)-1-methylpiperidin-3-yl]-6,7-dihydro-5H-cyclopenta[d]pyridazin-1-amine;
4-[2,4-bis(trifluoromethyl)phenyl]-N-[(3R)-1-methylpiperidin-3-yl]-6,7-dihydro-5H-cyclopenta[d]pyridazin-1-amine;
2-{1-[(4-methyl-1,4-oxazepan-6-yl)amino]pyrido[3,4-d]pyridazin-4-yl}-5-(trifluoromethyl)phenol;
2-(4-{[(3R)-azepan-3-yl]amino}pyrido[3,4-d]pyridazin-1-yl)-5-(trifluoromethyl)phenol;
2-{4-[(4-methyl-1,4-oxazepan-6-yl)amino]pyrido[3,4-d]pyridazin-1-yl}-5-(trifluoromethyl)phenol;
2-{4-[(1-methylazepan-4-yl)amino]pyrido[3,4-d]pyridazin-1-yl}-5-(trifluoromethyl)phenol;
2-{4-[(1,4-dimethyl-1,4-diazepan-6-yl)amino]pyrido[3,4-d]pyridazin-1-yl}-5-(trifluoromethyl)phenol;
2-(4-{[(3R)-5,5-difluoro-1-methylpiperidin-3-yl]amino}pyrido[3,4-d]pyridazin-1-yl)-5-(trifluoromethyl)phenol;
ethyl (2S,5R)-5-({1-[2-hydroxy-4-(trifluoromethyl)phenyl]pyrido[3,4-d]pyridazin-4-yl}amino)piperidine-2-carboxylate;
3-methoxy-5-methyl-2-(4-{[(3R)-1-methylpiperidin-3-yl]amino}pyrido[3,4-d]pyridazin-1-yl)phenol;
5-methyl-2-(4-{[(3R)-1-methylpiperidin-3-yl]amino}pyrido[3,4-d]pyridazin-1-yl)benzene-1,3-diol;
2-(4-{[(1R,3S)-3-hydroxycyclohexyl]amino}pyrido[3,4-d]pyridazin-1-yl)-5-(trifluoromethyl)phenol;
2-(4-{[(1R,3R)-3-hydroxycyclohexyl]amino}pyrido[3,4-d]pyridazin-1-yl)-5-(trifluoromethyl)phenol;
2-(4-{[(3R)-1-(oxetan-3-yl)piperidin-3-yl]amino}pyrido[3,4-d]pyridazin-1-yl)-5-(trifluoromethyl)phenol;
1-(4-cyclopropyl-2-methylphenyl)-N-[(3R)-1-methylpiperidin-3-yl]pyrido[3,4-d]pyridazin-4-amine;
3-fluoro-2-(4-{[(3R)-1-(2-hydroxyethyl)piperidin-3-yl]amino}pyrido[3,4-d]pyridazin-1-yl)-5-methylphenol;
3-cyclopropyl-2-(4-{[(3R)-1-methylpiperidin-3-yl]amino}pyrido[3,4-d]pyridazin-1-yl)-5-(trifluoromethyl)phenol;
2-(4-{[2-(dimethylamino)-2-methylpropyl]amino}pyrido[3,4-d]pyridazin-1-yl)-5-(trifluoromethyl)phenol;
N-{(3R)-1-[2-(difluoromethoxy)ethyl]piperidin-3-yl}-1-[2-methoxy-4-(trifluoromethyl)phenyl]pyrido[3,4-d]pyridazin-4-amine;
2-(4-{[(3R)-1-(2,2-difluoroethyl)piperidin-3-yl]amino}pyrido[3,4-d]pyridazin-1-yl)-5-(trifluoromethyl)phenol;
2-(4-{[(3 S,5S)-5-fluoro-1-methylpiperidin-3-yl]amino}pyrido[3,4-d]pyridazin-1-yl)-5-(trifluoromethyl)phenol;
2-(4-{[(3R)-1-(oxan-4-yl)piperidin-3-yl]amino}pyrido[3,4-d]pyridazin-1-yl)-5-(trifluoromethyl)phenol;
2-(4-{[(3R,5R)-5-fluoro-1-methylpiperidin-3-yl]amino}pyrido[3,4-d]pyridazin-1-yl)-5-(trifluoromethyl)phenol;
2-(4-{[(3R)-1-(oxolan-3-yl)piperidin-3-yl]amino}pyrido[3,4-d]pyridazin-1-yl)-5-(trifluoromethyl)phenol;
5-chloro-3-fluoro-2-(4-{[(3R)-1-(2-hydroxyethyl)piperidin-3-yl]amino}pyrido[3,4-d]pyridazin-1-yl)phenol;
2-(4-{[(3R)-1-(2,2,2-trifluoroethyl)piperidin-3-yl]amino}pyrido[3,4-d]pyridazin-1-yl)-5-(trifluoromethyl)phenol;
5-cyclopropyl-3-fluoro-2-(4-{[(3R)-1-(2-hydroxyethyl)piperidin-3-yl]amino}pyrido[3,4-d]pyridazin-1-yl)phenol;
3-fluoro-2-(4-{[(3R)-1-(2-hydroxyethyl)piperidin-3-yl]amino}pyrido[3,4-d]pyridazin-1-yl)phenol;
2-(4-{[(3R)-1-(2-hydroxy-2-methylpropyl)piperidin-3-yl]amino}pyrido[3,4-d]pyridazin-1-yl)-5-(trifluoromethyl)phenol;
1-[2-(difluoromethyl)-4-methoxyphenyl]-N-[(3R)-1-methylpiperidin-3-yl]pyrido[3,4-d]pyridazin-4-amine;
2-[4-(cyclohexylamino)pyrido[3,4-d]pyridazin-1-yl]-5-(trifluoromethyl)phenol;
1-[4-chloro-2-(trifluoromethyl)phenyl]-N-[(3R)-1-methylpiperidin-3-yl]pyrido[3,4-d]pyridazin-4-amine;
N-[(3R)-1-methylpiperidin-3-yl]-1-[2-methyl-4-(trifluoromethyl)phenyl]pyrido[3,4-d]pyridazin-4-amine;
N-[(3R)-1-methylpiperidin-3-yl]-1-{2-[(propan-2-yl)oxy]-4-(trifluoromethyl)phenyl}pyrido[3,4-d]pyridazin-4-amine;
2-(4-{methyl[(3R)-piperidin-3-yl]amino}pyrido[3,4-d]pyridazin-1-yl)-5-(trifluoromethyl)phenol;
2-[4-({(3R)-1-[2-(difluoromethoxy)ethyl]piperidin-3-yl}amino)pyrido[3,4-d]pyridazin-1-yl]-5-(trifluoromethyl)phenol;
2-(4-{[(3R)-1-(3-fluoropropyl)piperidin-3-yl]amino}pyrido[3,4-d]pyridazin-1-yl)-5-(trifluoromethyl)phenol;
2-(4-{[(3R)-1-(propan-2-yl)piperidin-3-yl]amino}pyrido[3,4-d]pyridazin-1-yl)-5-(trifluoromethyl)phenol;
1-[4-methoxy-2-(propan-2-yl)phenyl]-N-[(3S)-1-methylpiperidin-3-yl]pyrido[3,4-d]pyridazin-4-amine;
2-(4-{methyl[(3R)-1-methylpiperidin-3-yl]amino}pyrido[3,4-d]pyridazin-1-yl)-5-(trifluoromethyl)phenol;
N-[(3R)-1-methylpiperidin-3-yl]-1-[4-methyl-2-(trifluoromethyl)phenyl]pyrido[3,4-d]pyridazin-4-amine;
1-[2-(difluoromethyl)-4-methylphenyl]-N-[(3R)-1-methylpiperidin-3-yl]pyrido[3,4-d]pyridazin-4-amine;
2-(4-{[(3R)-1-(1-²H)cyclobutylpiperidin-3-yl]amino}pyrido[3,4-d]pyridazin-1-yl)-5-(trifluoromethyl)phenol;
2-(4-{[(3 S,4R)-4-fluoropiperidin-3-yl]amino}pyrido[3,4-d]pyridazin-1-yl)-5-(trifluoromethyl)phenol;
2-(4-{[(1-methylpiperidin-4-yl)methyl]amino}pyrido[3,4-d]pyridazin-1-yl)-5-(trifluoromethyl)phenol;
2-[4-({[1-(2-hydroxyethyl)piperidin-4-yl]methyl}amino)pyrido[3,4-d]pyridazin-1-yl]-5-(trifluoromethyl)phenol;
3-fluoro-5-methyl-2-(8-{[(3R)-1-methylpiperidin-3-yl]amino}pyrido[2,3-d]pyridazin-5-yl)phenol;
2-(4-{[(1s,3s)-3-hydroxy-3-methylcyclobutyl]amino}pyrido[3,4-d]pyridazin-1-yl)-5-(trifluoromethyl)phenol;
2-(4-{[(3R,5R)-5-fluoropiperidin-3-yl]amino}pyrido[3,4-d]pyridazin-1-yl)-5-(trifluoromethyl)phenol;
5-ethyl-2-(4-{[(3R)-1-methylpiperidin-3-yl]amino}pyrido[3,4-d]pyridazin-1-yl)phenol;
2-(4-{[(1R,3S)-3-methoxycyclohexyl]amino}pyrido[3,4-d]pyridazin-1-yl)-5-(trifluoromethyl)phenol;
2-(4-{[(3R)-oxan-3-yl]amino}pyrido[3,4-d]pyridazin-1-yl)-5-(trifluoromethyl)phenol;
2-(4-{[(3 S)-oxan-3-yl]amino}pyrido[3,4-d]pyridazin-1-yl)-5-(trifluoromethyl)phenol;
1-[2-(difluoromethoxy)-4-(trifluoromethyl)phenyl]-N-[(3R)-piperidin-3-yl]pyrido[3,4-d]pyridazin-4-amine;
N-[(3R)-1-cyclobutylpiperidin-3-yl]-1-[2-(difluoromethoxy)-4-(trifluoromethyl)phenyl]pyrido[3,4-d]pyridazin-4-amine;
2-(7-{[(3R)-1-methylpiperidin-3-yl]amino}furo[2,3-d]pyridazin-4-yl)-5-(trifluoromethyl)phenol;
5-chloro-2-(4-{[(3R)-1-methylpiperidin-3-yl]amino}-6,7-dihydro-5H-cyclopenta[d]pyridazin-1-yl)phenol;
2-(4-{[(3 S,4R)-4-fluoro-1-methylpiperidin-3-yl]amino}pyrido[3,4-d]pyridazin-1-yl)-5-(trifluoromethyl)phenol;
2-(4-{[(1r,3r)-3-hydroxy-3-methylcyclobutyl]amino}pyrido[3,4-d]pyridazin-1-yl)-5-(trifluoromethyl)phenol;
2-(4-{[(3R)-1-(2-hydroxyethyl)azepan-3-yl]amino}pyrido[3,4-d]pyridazin-1-yl)-5-(trifluoromethyl)phenol;
ethyl (2S,5R)-5-({1-[2-hydroxy-4-(trifluoromethyl)phenyl]pyrido[3,4-d]pyridazin-4-yl}amino)-1-methylpiperidine-2-carboxylate;
1-[2-(difluoromethyl)-4-(trifluoromethoxy)phenyl]-N-[(3R)-1-methylpiperidin-3-yl]pyrido[3,4-d]pyridazin-4-amine;
1-[2-(difluoromethyl)-5-(trifluoromethoxy)phenyl]-N-[(3R)-1-methylpiperidin-3-yl]pyrido[3,4-d]pyridazin-4-amine;
2-(8-methyl-4-{[(3R)-1-methylpiperidin-3-yl]amino}pyrrolo[1,2-d][1,2,4]triazin-1-yl)-5-(trifluoromethyl)phenol;
1-[2-(difluoromethoxy)-4-(trifluoromethyl)phenyl]-N-[(3R)-1-methylpiperidin-3-yl]pyrrolo[1,2-d][1,2,4]triazin-4-amine;
N-{(3R)-1-[2-(difluoromethoxy)ethyl]piperidin-3-yl}-1-[2-(difluoromethoxy)-4-(trifluoromethyl)phenyl]pyrrolo[1,2-d][1,2,4]triazin-4-amine;
N-[(3R)-azocan-3-yl]-1-[2-(difluoromethoxy)-4-(trifluoromethyl)phenyl]pyrrolo[1,2-d][1,2,4]triazin-4-amine;
2-[(3R)-3-({1-[2-(difluoromethoxy)-4-(trifluoromethyl)phenyl]pyrrolo[1,2-d][1,2,4]triazin-4-yl}amino)piperidin-1-yl]ethan-1-ol;
1-[2-(difluoromethoxy)-4-(trifluoromethyl)phenyl]-N-[(3R)-1-(oxan-4-yl)piperidin-3-yl]pyrrolo[1,2-d][1,2,4]triazin-4-amine;
5-cyclopropyl-2-(2-methyl-7-{[(3R)-1-methylpiperidin-3-yl]amino}pyrazolo[1,5-d][1,2,4]triazin-4-yl)phenol;
5-methoxy-2-(2-methyl-7-{[(3R)-1-methylpiperidin-3-yl]amino}pyrazolo[1,5-d][1,2,4]triazin-4-yl)phenol;
2-(2-methyl-7-{[(3R)-1-methylpiperidin-3-yl]amino}pyrazolo[1,5-d][1,2,4]triazin-4-yl)-5-(trifluoromethyl)phenol;
4-[2-(difluoromethoxy)-4-(trifluoromethyl)phenyl]-2-methyl-N-[(3R)-1-methylpiperidin-3-yl]pyrazolo[1,5-d][1,2,4]triazin-7-amine;
5-cyclopropyl-2-(4-{[(3R)-1-methylpiperidin-3-yl]amino}pyrrolo[1,2-d][1,2,4]triazin-1-yl)phenol;
2-(2-methyl-4-{[(3R)-piperidin-3-yl]amino}pyrazolo[1,5-d][1,2,4]triazin-7-yl)-5-(trifluoromethyl)phenol;
2-(4-{[(3R)-1-methylpiperidin-3-yl]amino}imidazo[1,5-d][1,2,4]triazin-1-yl)-5-(trifluoromethyl)phenol;
5-bromo-2-(2-methyl-7-{[(3R)-1-methylpiperidin-3-yl]amino}pyrazolo[1,5-d][1,2,4]triazin-4-yl)phenol;
1-[2-(difluoromethoxy)-4-(trifluoromethyl)phenyl]-N-[(3R)-1-ethylpiperidin-3-yl]pyrrolo[1,2-d][1,2,4]triazin-4-amine;
2-(2-methyl-4-{[(3R)-1-methylpiperidin-3-yl]amino}pyrazolo[1,5-d][1,2,4]triazin-7-yl)-5-(trifluoromethyl)phenol;
5-methyl-2-(4-{[(3R)-1-methylpiperidin-3-yl]amino}pyrrolo[1,2-d][1,2,4]triazin-1-yl)phenol;
2-(4-{[(3R)-1-(2-hydroxyethyl)piperidin-3-yl]amino}-2-methylpyrazolo[1,5-d][1,2,4]triazin-7-yl)-5-(trifluoromethyl)phenol;
5-methoxy-2-(2-methyl-4-{[(3R)-piperidin-3-yl]amino}pyrazolo[1,5-d][1,2,4]triazin-7-yl)phenol;
5-cyclopropyl-2-(4-{[(3R)-1-(oxan-4-yl)piperidin-3-yl]amino}pyrrolo[1,2-d][1,2,4]triazin-1-yl)phenol;
3-fluoro-2-(4-{[(3R)-1-(2-hydroxyethyl)piperidin-3-yl]amino}pyrrolo[1,2-d][1,2,4]triazin-1-yl)-5-methylphenol;
3-fluoro-5-methyl-2-(4-{[(3R)-1-methylpiperidin-3-yl]amino}pyrrolo[1,2-d][1,2,4]triazin-1-yl)phenol;
2-(4-{[(3R)-1-(oxan-4-yl)piperidin-3-yl]amino}pyrrolo[1,2-d][1,2,4]triazin-1-yl)-5-(trifluoromethyl)phenol;
2-[4-({(3R)-1-[2-(difluoromethoxy)ethyl]piperidin-3-yl}amino)pyrrolo[1,2-d][1,2,4]triazin-1-yl]-3-fluoro-5-methylphenol;
2-(4-{[(6S)-4-methyl-4-azaspiro[2.5]octan-6-yl]amino}pyrido[3,4-d]pyridazin-1-yl)-5-(trifluoromethyl)phenol;
2-(2-cyclopropyl-7-{[(3R)-1-methylpiperidin-3-yl]amino}pyrazolo[1,5-d][1,2,4]triazin-4-yl)-5-(trifluoromethyl)phenol;
2-(2-cyclopropyl-7-{[(3R)-1-methylpiperidin-3-yl]amino}pyrazolo[1,5-d][1,2,4]triazin-4-yl)-5-methoxyphenol;
1-[2,4-bis(trifluoromethyl)phenyl]-N-[(3R)-1-methylpiperidin-3-yl]pyrrolo[1,2-d][1,2,4]triazin-4-amine;
1-[2-methoxy-4-(trifluoromethyl)phenyl]-N-[(3R)-1-methylpiperidin-3-yl]imidazo[1,5-d][1,2,4]triazin-4-amine;
2-(4-{[(3R)-1-(2-hydroxyethyl)piperidin-3-yl]amino}imidazo[1,5-d][1,2,4]triazin-1-yl)-5-(trifluoromethyl)phenol;
5-methoxy-2-(2-methyl-4-{[(3R)-1-methylpiperidin-3-yl]amino}pyrazolo[1,5-d][1,2,4]triazin-7-yl)phenol;
2-(4-{[(3R)-piperidin-3-yl]amino}pyrrolo[1,2-d][1,2,4]triazin-1-yl)-5-(trifluoromethyl)phenol;
2-(4-{[(3R)-1-(2-hydroxyethyl)piperidin-3-yl]amino}pyrrolo[1,2-d][1,2,4]triazin-1-yl)-5-(trifluoromethyl)phenol;
2-(4-{[(3R)-1-(2-hydroxyethyl)piperidin-3-yl]amino}pyrrolo[1,2-d][1,2,4]triazin-1-yl)-5-methylphenol;
5-chloro-2-(4-{[(3R)-1-methylpiperidin-3-yl]amino}pyrrolo[1,2-d][1,2,4]triazin-1-yl)phenol;
1-[4-chloro-2-(difluoromethoxy)phenyl]-N-[(3R)-1-methylpiperidin-3-yl]pyrrolo[1,2-d][1,2,4]triazin-4-amine;
5-methoxy-2-(4-{[(3R)-1-methylpiperidin-3-yl]amino}pyrrolo[1,2-d][1,2,4]triazin-1-yl)phenol;
2-(4-{[(3R)-1-(2-hydroxyethyl)piperidin-3-yl]amino}pyrrolo[1,2-d][1,2,4]triazin-1-yl)-5-methoxyphenol;
3-fluoro-5-methyl-2-(2-methyl-7-{[(3R)-1-methylpiperidin-3-yl]amino}pyrazolo[1,5-d][1,2,4]triazin-4-yl)phenol;
2-(4-{[(3R)-1-ethylpiperidin-3-yl]amino}imidazo[1,5-d][1,2,4]triazin-1-yl)-5-(trifluoromethyl)phenol;
(1S,3R)-3-({1-[2-(difluoromethoxy)-4-(trifluoromethyl)phenyl]pyrrolo[1,2-d][1,2,4]triazin-4-yl}amino)cyclohexan-1-ol;
1-[2-(difluoromethoxy)-4-(trifluoromethyl)phenyl]-N-[(1R,3S)-3-methoxycyclohexyl]pyrrolo[1,2-d][1,2,4]triazin-4-amine;
3-fluoro-2-(2-methyl-7-{[(3R)-1-methylpiperidin-3-yl]amino}pyrazolo[1,5-d][1,2,4]triazin-4-yl)-5-(trifluoromethyl)phenol;
2-(4-{[(3R)-1-(2-hydroxy-2-methylpropyl)piperidin-3-yl]amino}pyrrolo[1,2-d][1,2,4]triazin-1-yl)-5-(trifluoromethyl)phenol;
2-(4-{[(3R)-piperidin-3-yl]amino}pyrrolo[1,2-d][1,2,4]triazin-1-yl)-5-(trifluoromethoxy)phenol;
2-(4-{[(3R)-1-methylpiperidin-3-yl]amino}pyrrolo[1,2-d][1,2,4]triazin-1-yl)-5-(trifluoromethoxy)phenol;
2-(4-{[(3R)-1-(2-hydroxy-2-methylpropyl)piperidin-3-yl]amino}pyrrolo[1,2-d][1,2,4]triazin-1-yl)-5-methylphenol;
5-chloro-2-(4-{[(3R)-1-(2,2-difluoroethyl)piperidin-3-yl]amino}pyrrolo[1,2-d][1,2,4]triazin-1-yl)phenol;
5-chloro-2-(4-{[(3R)-1-(2-hydroxyethyl)piperidin-3-yl]amino}pyrrolo[1,2-d][1,2,4]triazin-1-yl)phenol;
1-[4-methoxy-2-(trifluoromethyl)phenyl]-N-[(3R)-1-methylpiperidin-3-yl]pyrrolo[1,2-d][1,2,4]triazin-4-amine;
1-[2,4-bis(trifluoromethyl)phenyl]-N-[(3R)-1-ethylpiperidin-3-yl]pyrrolo[1,2-d][1,2,4]triazin-4-amine;
3-fluoro-2-(4-{[(3R)-1-methylpiperidin-3-yl]amino}pyrrolo[1,2-d][1,2,4]triazin-1-yl)-5-(trifluoromethyl)phenol;
2-(4-{[(3R)-1-(2-hydroxyethyl)piperidin-3-yl]amino}pyrrolo[1,2-d][1,2,4]triazin-1-yl)-5-(trifluoromethoxy)phenol;
4-[4-bromo-2-(trifluoromethoxy)phenyl]-2-methyl-N-[(3R)-1-methylpiperidin-3-yl]pyrazolo[1,5-d][1,2,4]triazin-7-amine;
2-[(3R)-3-({4-[2-(difluoromethoxy)-4-(trifluoromethyl)phenyl]-2-methylpyrazolo[1,5-d][1,2,4]triazin-7-yl}amino)piperidin-1-yl]ethan-1-ol;
1-[2-(difluoromethoxy)-4-(trifluoromethyl)phenyl]-N-[(3R)-oxan-3-yl]pyrrolo[1,2-d][1,2,4]triazin-4-amine;
2-(4-{[(3R)-1-ethylpiperidin-3-yl]amino}pyrrolo[1,2-d][1,2,4]triazin-1-yl)-5-methoxyphenol;
(1R,3S)—Ni-{1-[2-(difluoromethoxy)-4-(trifluoromethyl)phenyl]pyrrolo[1,2-d][1,2,4]triazin-4-yl}cyclohexane-1,3-diamine;
5-cyclopropyl-3-fluoro-2-(4-{[(3R)-1-(2-hydroxyethyl)piperidin-3-yl]amino}pyrrolo[1,2-d][1,2,4]triazin-1-yl)phenol;
5-cyclopropyl-3-fluoro-2-(4-{[(3R)-1-methylpiperidin-3-yl]amino}pyrrolo[1,2-d][1,2,4]triazin-1-yl)phenol;
4-[4-methoxy-2-(trifluoromethyl)phenyl]-2-methyl-N-[(3R)-1-methylpiperidin-3-yl]pyrazolo[1,5-d][1,2,4]triazin-7-amine;
4-[2,4-bis(trifluoromethyl)phenyl]-2-methyl-N-[(3R)-1-methylpiperidin-3-yl]pyrazolo[1,5-d][1,2,4]triazin-7-amine;
1-(4-chloro-2-methoxyphenyl)-N-[(3R)-1-methylpiperidin-3-yl]imidazo[1,5-d][1,2,4]triazin-4-amine;
5-chloro-2-(4-{[(3R)-1-methylpiperidin-3-yl]amino}imidazo[1,5-d][1,2,4]triazin-1-yl)phenol;
(3R,5R)-5-({1-[2-(difluoromethoxy)-4-(trifluoromethyl)phenyl]pyrrolo[1,2-d][1,2,4]triazin-4-yl}amino)-1-methylpiperidin-3-ol;
8-[2-methoxy-4-(trifluoromethyl)phenyl]-N-[(3R)-1-methylpiperidin-3-yl]imidazo[1,2-d][1,2,4]triazin-5-amine;
(3R,5R)-5-{[1-(4-cyclopropyl-2-fluoro-6-hydroxyphenyl)pyrrolo[1,2-d][1,2,4]triazin-4-yl]amino}-1-methylpiperidin-3-ol;
1-[2-(difluoromethoxy)-4-(trifluoromethyl)phenyl]-N-[(3R)-oxolan-3-yl]pyrrolo[1,2-d][1,2,4]triazin-4-amine;
(2S)-1-({1-[2-(difluoromethoxy)-4-(trifluoromethyl)phenyl]pyrrolo[1,2-d][1,2,4]triazin-4-yl}amino)propan-2-ol;
4-(4-bromo-2-methoxyphenyl)-N-[(3R)-piperidin-3-yl]pyrazolo[1,5-d][1,2,4]triazin-7-amine;
5-bromo-2-(7-{[(3R)-piperidin-3-yl]amino}pyrazolo[1,5-d][1,2,4]triazin-4-yl)phenol;
N-[(3R)-1-ethylpiperidin-3-yl]-1-[4-methoxy-2-(trifluoromethyl)phenyl]pyrrolo[1,2-d][1,2,4]triazin-4-amine;
2-(8-fluoro-4-{[(3R)-1-methylpiperidin-3-yl]amino}pyrrolo[1,2-d][1,2,4]triazin-1-yl)-5-(trifluoromethyl)phenol;
1-[2-(difluoromethoxy)-4-(trifluoromethyl)phenyl]-8-fluoro-N-[(3R)-1-methylpiperidin-3-yl]pyrrolo[1,2-d][1,2,4]triazin-4-amine;
3-fluoro-2-(4-{[(3R)-oxan-3-yl]amino}pyrrolo[1,2-d][1,2,4]triazin-1-yl)-5-(trifluoromethyl)phenol;
2-methyl-N-[(3R)-1-methylpiperidin-3-yl]-4-[4-methyl-2-(trifluoromethoxy)phenyl]pyrazolo[1,5-d][1,2,4]triazin-7-amine;
2-(4-{[(3R)-oxan-3-yl]amino}imidazo[1,5-d][1,2,4]triazin-1-yl)-5-(trifluoromethyl)phenol;
2-(5-{[(3R)-1-methylpiperidin-3-yl]amino}imidazo[1,2-d][1,2,4]triazin-8-yl)-5-(trifluoromethyl)phenol;
2-(4-{[(1R,3S)-3-hydroxycyclohexyl]amino}pyrrolo[1,2-d][1,2,4]triazin-1-yl)-5-(trifluoromethyl)phenol;
2-(4-{[(1R,3S)-3-methoxycyclohexyl]amino}pyrrolo[1,2-d][1,2,4]triazin-1-yl)-5-(trifluoromethyl)phenol;
2-(4-{[(3R)-oxan-3-yl]amino}pyrrolo[1,2-d][1,2,4]triazin-1-yl)-5-(trifluoromethyl)phenol;
5-chloro-2-(4-{[(3R)-oxan-3-yl]amino}pyrrolo[1,2-d][1,2,4]triazin-1-yl)phenol;
5-chloro-2-(4-{[(3R)-1-(2-hydroxy-2-methylpropyl)piperidin-3-yl]amino}pyrrolo[1,2-d][1,2,4]triazin-1-yl)phenol;
5-chloro-2-(4-{[(3R)-1-(propan-2-yl)piperidin-3-yl]amino}pyrrolo[1,2-d][1,2,4]triazin-1-yl)phenol;
2-(4-{[(1s,3s)-3-hydroxy-3-(trifluoromethyl)cyclobutyl]amino}pyrrolo[1,2-d][1,2,4]triazin-1-yl)-5-(trifluoromethyl)phenol;
2-(4-{[(1s,3s)-3-hydroxy-3-methylcyclobutyl]amino}pyrrolo[1,2-d][1,2,4]triazin-1-yl)-5-(trifluoromethyl)phenol;
3-methoxy-4-(4-{[(3R)-1-methylpiperidin-3-yl]amino}pyrrolo[1,2-d][1,2,4]triazin-1-yl)benzonitrile;
2-(4-{[(3R,5R)-5-fluoropiperidin-3-yl]amino}pyrrolo[1,2-d][1,2,4]triazin-1-yl)-5-(trifluoromethoxy)phenol;
2-(4-{[(3R,5R)-5-fluoro-1-methylpiperidin-3-yl]amino}pyrrolo[1,2-d][1,2,4]triazin-1-yl)-5-(trifluoromethoxy)phenol;
2-(4-{[(3R,5R)-5-fluoro-1-methylpiperidin-3-yl]amino}pyrrolo[1,2-d][1,2,4]triazin-1-yl)-5-(trifluoromethyl)phenol;
2-(4-{[(3R)-1-(2,2-difluoroethyl)piperidin-3-yl]amino}pyrrolo[1,2-d][1,2,4]triazin-1-yl)-5-(trifluoromethyl)phenol;
1-[2-(difluoromethoxy)-4-(trifluoromethyl)phenyl]-N-[(3R,5R)-5-fluoro-1-methylpiperidin-3-yl]pyrrolo[1,2-d][1,2,4]triazin-4-amine;
2-(4-{[(3R)-1-ethylpiperidin-3-yl]amino}pyrrolo[1,2-d][1,2,4]triazin-1-yl)-3-fluoro-5-(trifluoromethyl)phenol;
3-fluoro-2-(4-{[(3R)-1-(2-hydroxyethyl)piperidin-3-yl]amino}pyrrolo[1,2-d][1,2,4]triazin-1-yl)-5-(trifluoromethyl)phenol
3-fluoro-2-(4-{[(3R,5R)-5-fluoro-1-methylpiperidin-3-yl]amino}pyrrolo[1,2-d][1,2,4]triazin-1-yl)-5-(trifluoromethyl)phenol;
3-fluoro-2-(4-{[(3R)-1-(2-hydroxy-2-methylpropyl)piperidin-3-yl]amino}pyrrolo[1,2-d][1,2,4]triazin-1-yl)-5-(trifluoromethyl)phenol;
4-[4-cyclopropyl-2-(trifluoromethoxy)phenyl]-2-methyl-N-[(3R)-1-methylpiperidin-3-yl]pyrazolo[1,5-d][1,2,4]triazin-7-amine;
2-methyl-N-[(3R)-1-methylpiperidin-3-yl]-4-[2-(trifluoromethoxy)phenyl]pyrazolo[1,5-d][1,2,4]triazin-7-amine;
3-fluoro-2-(4-{[(1s,3s)-3-hydroxy-3-methylcyclobutyl]amino}pyrrolo[1,2-d][1,2,4]triazin-1-yl)-5-(trifluoromethyl)phenol;
3-fluoro-5-methyl-2-(4-{[(3R)-oxan-3-yl]amino}pyrrolo[1,2-d][1,2,4]triazin-1-yl)phenol;
3-fluoro-2-(4-{[(1R,3S)-3-hydroxycyclohexyl]amino}pyrrolo[1,2-d][1,2,4]triazin-1-yl)-5-methylphenol;
5-cyclopropyl-3-fluoro-2-(4-{[(3R)-oxan-3-yl]amino}pyrrolo[1,2-d][1,2,4]triazin-1-yl)phenol;
5-cyclopropyl-3-fluoro-2-(4-{[(1R,3S)-3-hydroxycyclohexyl]amino}pyrrolo[1,2-d][1,2,4]triazin-1-yl)phenol;
1-[2-(difluoromethyl)-4-methylphenyl]-N-[(3R)-1-methylpiperidin-3-yl]pyrrolo[1,2-d][1,2,4]triazin-4-amine;
1-[2-(difluoromethyl)-4-(trifluoromethyl)phenyl]-N-[(3R)-1-methylpiperidin-3-yl]pyrrolo[1,2-d][1,2,4]triazin-4-amine;
(1s,3s)-3-({1-[2-(difluoromethyl)-4-(trifluoromethyl)phenyl]pyrrolo[1,2-d][1,2,4]triazin-4-yl}amino)-1-methylcyclobutan-1-ol;
1-[2-(difluoromethoxy)-6-fluoro-4-methylphenyl]-N-[(3R)-1-methylpiperidin-3-yl]pyrrolo[1,2-d][1,2,4]triazin-4-amine;
5-cyclopropyl-3-fluoro-2-(2-methyl-7-{[(3R)-1-methylpiperidin-3-yl]amino}pyrazolo[1,5-d][1,2,4]triazin-4-yl)phenol;
3-fluoro-2-(4-{[(3R)-1-(oxan-4-yl)piperidin-3-yl]amino}pyrrolo[1,2-d][1,2,4]triazin-1-yl)-5-(trifluoromethyl)phenol;
3-methyl-2-(4-{[(3R)-1-methylpiperidin-3-yl]amino}pyrrolo[1,2-d][1,2,4]triazin-1-yl)-5-(trifluoromethyl)phenol;
5-chloro-3-fluoro-2-(4-{[(3R)-1-methylpiperidin-3-yl]amino}pyrrolo[1,2-d][1,2,4]triazin-1-yl)phenol;
5-chloro-2-(4-{[(3R)-1-ethylpiperidin-3-yl]amino}pyrrolo[1,2-d][1,2,4]triazin-1-yl)-3-fluorophenol;
5-chloro-3-fluoro-2-(4-{[(1s,3s)-3-hydroxy-3-methylcyclobutyl]amino}pyrrolo[1,2-d][1,2,4]triazin-1-yl)phenol;
5-chloro-2-(4-{[(3R)-piperidin-3-yl]amino}imidazo[1,5-d][1,2,4]triazin-1-yl)phenol;
5-chloro-2-(4-{[(3R)-1-(2-hydroxyethyl)piperidin-3-yl]amino}imidazo[1,5-d][1,2,4]triazin-1-yl)phenol;
2-(4-{[(2R)-2-hydroxypropyl]amino}pyrrolo[1,2-d][1,2,4]triazin-1-yl)-5-(trifluoromethoxy)phenol;
2-{4-[(2-hydroxy-2-methylpropyl)amino]pyrrolo[1,2-d][1,2,4]triazin-1-yl}-5-(trifluoromethoxy)phenol;
1-[2-(difluoromethoxy)-4-(trifluoromethyl)phenyl]-8-fluoro-N-[(3R,5R)-5-fluoro-1-methylpiperidin-3-yl]pyrrolo[1,2-d][1,2,4]triazin-4-amine;
(2R)-1-({1-[2-(difluoromethoxy)-4-(trifluoromethyl)phenyl]-8-fluoropyrrolo[1,2-d][1,2,4]triazin-4-yl}amino)propan-2-ol;
2-(8-fluoro-4-{[(3R)-1-methylpiperidin-3-yl]amino}pyrrolo[1,2-d][1,2,4]triazin-1-yl)-5-(trifluoromethoxy)phenol;
5-chloro-2-(4-{[(3R)-1-ethylpiperidin-3-yl]amino}imidazo[1,5-d][1,2,4]triazin-1-yl)phenol;
1-[4-chloro-2-(difluoromethyl)phenyl]-N-[(3R)-1-methylpiperidin-3-yl]pyrrolo[1,2-d][1,2,4]triazin-4-amine;
3-fluoro-2-(4-{[(3R,5R)-5-fluoro-1-methylpiperidin-3-yl]amino}pyrrolo[1,2-d][1,2,4]triazin-1-yl)-5-methylphenol;
5-cyclopropyl-3-fluoro-2-(4-{[(3R,5R)-5-fluoro-1-methylpiperidin-3-yl]amino}pyrrolo[1,2-d][1,2,4]triazin-1-yl)phenol;
1-[4-cyclopropyl-2-(difluoromethyl)phenyl]-N-[(3R)-1-methylpiperidin-3-yl]pyrrolo[1,2-d][1,2,4]triazin-4-amine;
3-fluoro-2-(4-{[(3R)-1-(oxolan-3-yl)piperidin-3-yl]amino}pyrrolo[1,2-d][1,2,4]triazin-1-yl)-5-(trifluoromethyl)phenol;
2-(4-{[(1R,3S)-3-hydroxycyclopentyl]amino}pyrrolo[1,2-d][1,2,4]triazin-1-yl)-5-(trifluoromethyl)phenol;
2-(4-{[(1R,3R)-3-hydroxycyclopentyl]amino}pyrrolo[1,2-d][1,2,4]triazin-1-yl)-5-(trifluoromethyl)phenol;
2-{4-[(3-hydroxy-3-methylcyclohexyl)amino]pyrrolo[1,2-d][1,2,4]triazin-1-yl}-5-(trifluoromethyl)phenol;
4-(4-bromo-2-methoxyphenyl)-N-[(3R)-1-methylpiperidin-3-yl]pyrazolo[1,5-d][1,2,4]triazin-7-amine;
5-bromo-2-(7-{[(3R)-1-methylpiperidin-3-yl]amino}pyrazolo[1,5-d][1,2,4]triazin-4-yl)phenol;
5-bromo-2-(7-{[(3R)-1-ethylpiperidin-3-yl]amino}pyrazolo[1,5-d][1,2,4]triazin-4-yl)phenol;
2-[(3R)-3-({1-[4-methoxy-2-(trifluoromethyl)phenyl]pyrrolo[1,2-d][1,2,4]triazin-4-yl}amino)piperidin-1-yl]ethan-1-ol;
5-chloro-3-fluoro-2-(2-methyl-7-{[(3R)-1-methylpiperidin-3-yl]amino}pyrazolo[1,5-d][1,2,4]triazin-4-yl)phenol;
3-fluoro-5-methoxy-2-(4-{[(3R)-1-methylpiperidin-3-yl]amino}pyrrolo[1,2-d][1,2,4]triazin-1-yl)phenol;
3-fluoro-2-(4-{[(3R)-1-(2-hydroxyethyl)piperidin-3-yl]amino}pyrrolo[1,2-d][1,2,4]triazin-1-yl)-5-methoxyphenol;
1-[2-(difluoromethoxy)-4-(trifluoromethyl)phenyl]-N-[(3S)-oxolan-3-yl]pyrrolo[1,2-d][1,2,4]triazin-4-amine;
2-(4-{[(3 S)-oxolan-3-yl]amino}pyrrolo[1,2-d][1,2,4]triazin-1-yl)-5-(trifluoromethyl)phenol;
2-(4-{[(3R)-1-methyl-1,2,3,6-tetrahydropyridin-3-yl]amino}imidazo[1,5-d][1,2,4]triazin-1-yl)-5-(trifluoromethyl)phenol;
2-(7-{[(3R)-1-methylpiperidin-3-yl]amino}[1,2,3]triazolo[1,5-d][1,2,4]triazin-4-yl)-5-(trifluoromethyl)phenol;
1-[2-(difluoromethoxy)-6-fluoro-4-methylphenyl]-N-[(3R,5R)-5-fluoro-1-methylpiperidin-3-yl]pyrrolo[1,2-d][1,2,4]triazin-4-amine;
2-[(3R)-3-({1-[2-(difluoromethoxy)-6-fluoro-4-methylphenyl]pyrrolo[1,2-d][1,2,4]triazin-4-yl}amino)piperidin-1-yl]ethan-1-ol;
1-[2-(difluoromethoxy)-6-fluoro-4-methylphenyl]-N-[(3R)-oxan-3-yl]pyrrolo[1,2-d][1,2,4]triazin-4-amine;
(1s,3s)-3-({1-[2-(difluoromethoxy)-6-fluoro-4-methylphenyl]pyrrolo[1,2-d][1,2,4]triazin-4-yl}amino)-1-methylcyclobutan-1-ol;
3-fluoro-2-(4-{[(1s,3s)-3-hydroxy-3-methylcyclobutyl]amino}pyrrolo[1,2-d][1,2,4]triazin-1-yl)-5-methylphenol;
5-cyclopropyl-3-fluoro-2-(4-{[(1s,3s)-3-hydroxy-3-methylcyclobutyl]amino}pyrrolo[1,2-d][1,2,4]triazin-1-yl)phenol;
1-[2,4-bis(trifluoromethyl)phenyl]-N-[(3R,5R)-5-fluoro-1-methylpiperidin-3-yl]pyrrolo[1,2-d][1,2,4]triazin-4-amine;
1-[2,4-bis(trifluoromethyl)phenyl]-N-{(3R)-1-[2-(difluoromethoxy)ethyl]piperidin-3-yl}pyrrolo[1,2-d][1,2,4]triazin-4-amine;
1-[(3R)-3-({1-[2,4-bis(trifluoromethyl)phenyl]pyrrolo[1,2-d][1,2,4]triazin-4-yl}amino)piperidin-1-yl]-2-methylpropan-2-ol;
1-[2-(difluoromethoxy)-4-methoxyphenyl]-N-[(3R)-1-methylpiperidin-3-yl]pyrrolo[1,2-d][1,2,4]triazin-4-amine;
(1r,3r)-3-({1-[2-(difluoromethoxy)-4-methoxyphenyl]pyrrolo[1,2-d][1,2,4]triazin-4-yl}amino)-1-methylcyclobutan-1-ol;
2-(4-{[(2S)-2-hydroxypropyl]amino}pyrrolo[1,2-d][1,2,4]triazin-1-yl)-5-(trifluoromethyl)phenol;
2-(4-{[(3R)-oxolan-3-yl]amino}pyrrolo[1,2-d][1,2,4]triazin-1-yl)-5-(trifluoromethyl)phenol;
1-[2-(difluoromethoxy)-6-fluoro-4-methylphenyl]-N-[(3R)-1-(oxan-4-yl)piperidin-3-yl]pyrrolo[1,2-d][1,2,4]triazin-4-amine;
1-[(3R)-3-({1-[2-(difluoromethoxy)-6-fluoro-4-methylphenyl]pyrrolo[1,2-d][1,2,4]triazin-4-yl}amino)piperidin-1-yl]-2-methylpropan-2-ol;
3,5-dimethyl-2-(4-{[(3R)-1-methylpiperidin-3-yl]amino}pyrrolo[1,2-d][1,2,4]triazin-1-yl)phenol;
2-(4-{[(3 S,4S)-4-fluoro-1-methylpiperidin-3-yl]amino}pyrrolo[1,2-d][1,2,4]triazin-1-yl)-5-(trifluoromethyl)phenol;
2-(4-{[(3R)-1-ethylpiperidin-3-yl]amino}pyrrolo[1,2-d][1,2,4]triazin-1-yl)-5-(trifluoromethyl)phenol;
2-(4-{[(3R)-1-(propan-2-yl)piperidin-3-yl]amino}pyrrolo[1,2-d][1,2,4]triazin-1-yl)-5-(trifluoromethyl)phenol;
5-chloro-3-fluoro-2-(4-{[(3R,5R)-5-fluoro-1-methylpiperidin-3-yl]amino}pyrrolo[1,2-d][1,2,4]triazin-1-yl)phenol;
5-chloro-3-fluoro-2-(4-{[(3R)-1-(2-hydroxyethyl)piperidin-3-yl]amino}pyrrolo[1,2-d][1,2,4]triazin-1-yl)phenol;
2-(4-{[(3R)-1-(2,2,2-trifluoroethyl)piperidin-3-yl]amino}pyrrolo[1,2-d][1,2,4]triazin-1-yl)-5-(trifluoromethyl)phenol;
5-bromo-2-(4-{[(3R)-1-methylpiperidin-3-yl]amino}pyrrolo[1,2-d][1,2,4]triazin-1-yl)phenol;
1-[4-chloro-2-(2,2,2-trifluoroethoxy)phenyl]-N-[(3R)-1-methylpiperidin-3-yl]pyrrolo[1,2-d][1,2,4]triazin-4-amine;
2-(4-{[(3 S,4R)-4-fluoro-1-methylpiperidin-3-yl]amino}pyrrolo[1,2-d][1,2,4]triazin-1-yl)-5-(trifluoromethyl)phenol;
1-[2-(difluoromethoxy)-6-fluoro-4-(trifluoromethyl)phenyl]-N-[(3R)-1-methylpiperidin-3-yl]pyrrolo[1,2-d][1,2,4]triazin-4-amine;
1-[2-(difluoromethoxy)-6-fluoro-4-methylphenyl]-N-[(3R)-1-(oxolan-3-yl)piperidin-3-yl]pyrrolo[1,2-d][1,2,4]triazin-4-amine;
3-fluoro-5-methyl-2-(4-{[(3R)-1-(oxolan-3-yl)piperidin-3-yl]amino}pyrrolo[1,2-d][1,2,4]triazin-1-yl)phenol;
5-bromo-2-(4-{[(3R)-piperidin-3-yl]amino}pyrrolo[1,2-d][1,2,4]triazin-1-yl)phenol;
2-[(3R)-3-({1-[2,4-bis(trifluoromethyl)phenyl]pyrrolo[1,2-d][1,2,4]triazin-4-yl}amino)piperidin-1-yl]ethan-1-ol;
(2R)-1-({1-[2,4-bis(trifluoromethyl)phenyl]pyrrolo[1,2-d][1,2,4]triazin-4-yl}amino)propan-2-ol;
1-[2-(difluoromethoxy)-4-(trifluoromethyl)phenyl]-N-[(3R)-1-(propan-2-yl)piperidin-3-yl]pyrrolo[1,2-d][1,2,4]triazin-4-amine;
1-[2-(difluoromethoxy)-4-(trifluoromethyl)phenyl]-N-[(3R)-1-(2,2,2-trifluoroethyl)piperidin-3-yl]pyrrolo[1,2-d][1,2,4]triazin-4-amine;
5-cyclopropyl-2-(4-{[(3R)-1-ethylpiperidin-3-yl]amino}pyrrolo[1,2-d][1,2,4]triazin-1-yl)-3-fluorophenol;
5-cyclopropyl-3-fluoro-2-(4-{[(2R)-2-hydroxypropyl]amino}pyrrolo[1,2-d][1,2,4]triazin-1-yl)phenol;
1-[4-chloro-2-(trifluoromethyl)phenyl]-N-[(3R)-1-methylpiperidin-3-yl]pyrrolo[1,2-d][1,2,4]triazin-4-amine;
2-(4-{[(3R)-1-ethylpiperidin-3-yl]amino}pyrrolo[1,2-d][1,2,4]triazin-1-yl)-3-fluoro-5-methylphenol;
3-fluoro-2-(4-{[(2R)-2-hydroxypropyl]amino}pyrrolo[1,2-d][1,2,4]triazin-1-yl)-5-methylphenol;
5-cyclopropyl-3-fluoro-2-(4-{[(2S)-2-hydroxypropyl]amino}pyrrolo[1,2-d][1,2,4]triazin-1-yl)phenol;
5-[(²H₃)methyloxy]-2-(4-{[(3R)-1-methylpiperidin-3-yl]amino}pyrrolo[1,2-d][1,2,4]triazin-1-yl)phenol;
3-fluoro-2-(4-{[(3R,5R)-5-fluoro-1-methylpiperidin-3-yl]amino}pyrrolo[1,2-d][1,2,4]triazin-1-yl)-5-methoxyphenol;
1-[4-bromo-2-(trifluoromethoxy)phenyl]-N-[(3R)-1-methylpiperidin-3-yl]pyrrolo[1,2-d][1,2,4]triazin-4-amine;
1-[4-chloro-2-(difluoromethyl)phenyl]-N-[(3R)-1-ethylpiperidin-3-yl]pyrrolo[1,2-d][1,2,4]triazin-4-amine;
2-[(3R)-3-({1-[4-chloro-2-(difluoromethyl)phenyl]pyrrolo[1,2-d][1,2,4]triazin-4-yl}amino)piperidin-1-yl]ethan-1-ol;
1-[4-cyclopropyl-2-(difluoromethyl)phenyl]-N-[(3R,5R)-5-fluoro-1-methylpiperidin-3-yl]pyrrolo[1,2-d][1,2,4]triazin-4-amine;
1-[4-cyclopropyl-2-(difluoromethyl)phenyl]-N-[(3R)-1-ethylpiperidin-3-yl]pyrrolo[1,2-d][1,2,4]triazin-4-amine;
2-[(3R)-3-({1-[4-cyclopropyl-2-(difluoromethyl)phenyl]pyrrolo[1,2-d][1,2,4]triazin-4-yl}amino)piperidin-1-yl]ethan-1-ol;
5-cyclopropyl-2-(4-{[(3R)-1-(2,2-difluoroethyl)piperidin-3-yl]amino}pyrrolo[1,2-d][1,2,4]triazin-1-yl)-3-fluorophenol;
2-(4-{[(3R)-1-(2,2-difluoroethyl)piperidin-3-yl]amino}pyrrolo[1,2-d][1,2,4]triazin-1-yl)-3-fluoro-5-methylphenol;
2-(4-{[(1R,3S)-3-hydroxycyclohexyl]amino}imidazo[1,5-d][1,2,4]triazin-1-yl)-5-(trifluoromethyl)phenol;
3-fluoro-5-[(2H₃)methyloxy]-2-(4-{[(3R)-1-methylpiperidin-3-yl]amino}pyrrolo[1,2-d][1,2,4]triazin-1-yl)phenol;
2-(4-{[(3R)-1-ethylpiperidin-3-yl]amino}pyrrolo[1,2-d][1,2,4]triazin-1-yl)-3-fluoro-5-[(2H₃)methyloxy]phenol;
2-(7-{[(3R)-1-ethylpiperidin-3-yl]amino}-2-methylpyrazolo[1,5-d][1,2,4]triazin-4-yl)-3-fluoro-5-methylphenol;
1-[2,4-bis(trifluoromethyl)phenyl]-N-[(3R)-1-(oxan-4-yl)piperidin-3-yl]pyrrolo[1,2-d][1,2,4]triazin-4-amine;
1-[2,4-bis(trifluoromethyl)phenyl]-N-[(3R)-1-(oxolan-3-yl)piperidin-3-yl]pyrrolo[1,2-d][1,2,4]triazin-4-amine;
5-chloro-2-(4-{[(3R)-oxan-3-yl]amino}imidazo[1,5-d][1,2,4]triazin-1-yl)phenol;
1-[2-(difluoromethoxy)-4-(trifluoromethoxy)phenyl]-N-[(3R)-1-methylpiperidin-3-yl]pyrrolo[1,2-d][1,2,4]triazin-4-amine;
2-[(3R)-3-({1-[2-(difluoromethoxy)-4-(trifluoromethoxy)phenyl]pyrrolo[1,2-d][1,2,4]triazin-4-yl}amino)piperidin-1-yl]ethan-1-ol;
1-[2-(difluoromethoxy)-4-(trifluoromethoxy)phenyl]-N-[(3R,5R)-5-fluoro-1-methylpiperidin-3-yl]pyrrolo[1,2-d][1,2,4]triazin-4-amine;
(1s,3s)-3-({1-[2-(difluoromethoxy)-4-(trifluoromethoxy)phenyl]pyrrolo[1,2-d][1,2,4]triazin-4-yl}amino)-1-methylcyclobutan-1-ol;
2-(4-{[(1s,3s)-3-hydroxy-3-methylcyclobutyl]amino}pyrrolo[1,2-d][1,2,4]triazin-1-yl)-5-(trifluoromethoxy)phenol;
(1S,3R)-3-({1-[2-(difluoromethoxy)-4-(trifluoromethyl)phenyl]-8-fluoropyrrolo[1,2-d][1,2,4]triazin-4-yl}amino)cyclohexan-1-ol;
(1s,3s)-3-({1-[2-(difluoromethoxy)-4-(trifluoromethyl)phenyl]-8-fluoropyrrolo[1,2-d][1,2,4]triazin-4-yl}amino)-1-methylcyclobutan-1-ol;
2-(8-fluoro-4-{[(1R,3S)-3-hydroxycyclohexyl]amino}pyrrolo[1,2-d][1,2,4]triazin-1-yl)-5-(trifluoromethyl)phenol;
2-(4-{[(1s,3s)-3-hydroxy-3-methylcyclobutyl]amino}imidazo[1,5-d][1,2,4]triazin-1-yl)-5-(trifluoromethyl)phenol;
2-(4-{[(3R,5R)-5-fluoro-1-methylpiperidin-3-yl]amino}imidazo[1,5-d][1,2,4]triazin-1-yl)-5-(trifluoromethyl)phenol;
5-bromo-2-(4-{[(3R)-1-(2-hydroxyethyl)piperidin-3-yl]amino}pyrrolo[1,2-d][1,2,4]triazin-1-yl)phenol;
1-[2,4-bis(trifluoromethyl)phenyl]-N-[(3R)-1-(propan-2-yl)piperidin-3-yl]pyrrolo[1,2-d][1,2,4]triazin-4-amine;
1-({1-[2,4-bis(trifluoromethyl)phenyl]pyrrolo[1,2-d][1,2,4]triazin-4-yl}amino)-2-methylpropan-2-ol;
(1s,3s)-3-({1-[2,4-bis(trifluoromethyl)phenyl]pyrrolo[1,2-d][1,2,4]triazin-4-yl}amino)-1-methylcyclobutan-1-ol;
N-[(3R)-1-methylpiperidin-3-yl]-1-[4-methyl-2-(trifluoromethyl)phenyl]pyrrolo[1,2-d][1,2,4]triazin-4-amine;
1-[4-cyclopropyl-2-(trifluoromethyl)phenyl]-N-[(3R)-1-methylpiperidin-3-yl]pyrrolo[1,2-d][1,2,4]triazin-4-amine;
1-[2,4-bis(trifluoromethyl)phenyl]-N-[(3R,5R)-1-ethyl-5-fluoropiperidin-3-yl]pyrrolo[1,2-d][1,2,4]triazin-4-amine;
1-({1-[2-(difluoromethoxy)-4-(trifluoromethyl)phenyl]pyrrolo[1,2-d][1,2,4]triazin-4-yl}amino)-2-methylpropan-2-ol;
(1s,3s)-3-({1-[2-(difluoromethoxy)-4-(trifluoromethyl)phenyl]pyrrolo[1,2-d][1,2,4]triazin-4-yl}amino)-1-methylcyclobutan-1-ol;
1-[2-(difluoromethoxy)-4-(trifluoromethyl)phenyl]-N-[(3R,5R)-1-ethyl-5-fluoropiperidin-3-yl]pyrrolo[1,2-d][1,2,4]triazin-4-amine;
3-fluoro-2-{4-[(2-hydroxy-2-methylpropyl)amino]pyrrolo[1,2-d][1,2,4]triazin-1-yl}-5-methylphenol;
5-cyclopropyl-3-fluoro-2-{4-[(2-hydroxy-2-methylpropyl)amino]pyrrolo[1,2-d][1,2,4]triazin-1-yl}phenol;
5-cyclopropyl-3-fluoro-2-(4-{[(3R)-1-(propan-2-yl)piperidin-3-yl]amino}pyrrolo[1,2-d][1,2,4]triazin-1-yl)phenol;
5-cyclopropyl-3-fluoro-2-(4-{[(3R)-1-(2,2,2-trifluoroethyl)piperidin-3-yl]amino}pyrrolo[1,2-d][1,2,4]triazin-1-yl)phenol;
2-(6-methyl-4-{[(3R)-1-methylpiperidin-3-yl]amino}imidazo[1,5-d][1,2,4]triazin-1-yl)-5-(trifluoromethyl)phenol;
5-bromo-2-(7-{[(1s,3s)-3-hydroxy-3-methylcyclobutyl]amino}pyrazolo[1,5-d][1,2,4]triazin-4-yl)phenol;
4-[2-(benzyloxy)-4-bromophenyl]-N-[(3R)-1-methylpiperidin-3-yl]pyrazolo[1,5-d][1,2,4]triazin-7-amine;
5-bromo-2-(7-{[(3R,5R)-5-fluoro-1-methylpiperidin-3-yl]amino}pyrazolo[1,5-d][1,2,4]triazin-4-yl)phenol;
5-bromo-2-(7-{[(3R)-oxan-3-yl]amino}pyrazolo[1,5-d][1,2,4]triazin-4-yl)phenol;
5-bromo-2-(7-{[(3R)-1-(2,2-difluoroethyl)piperidin-3-yl]amino}pyrazolo[1,5-d][1,2,4]triazin-4-yl)phenol;
5-chloro-2-(4-{[(1s,3s)-3-hydroxy-3-methylcyclobutyl]amino}imidazo[1,5-d][1,2,4]triazin-1-yl)phenol;
5-methyl-2-(2-methyl-7-{[(3R)-1-methylpiperidin-3-yl]amino}pyrazolo[1,5-d][1,2,4]triazin-4-yl)phenol;
3-fluoro-5-methoxy-2-(2-methyl-7-{[(3R)-1-methylpiperidin-3-yl]amino}pyrazolo[1,5-d][1,2,4]triazin-4-yl)phenol;
5-bromo-2-(4-{[(3R)-1-(1-hydroxypropan-2-yl)piperidin-3-yl]amino}pyrrolo[1,2-d][1,2,4]triazin-1-yl)phenol;
5-chloro-2-(4-{[(3R)-1-(propan-2-yl)piperidin-3-yl]amino}imidazo[1,5-d][1,2,4]triazin-1-yl)phenol;
5-cyclopropyl-2-(7-{[(3R)-1-ethylpiperidin-3-yl]amino}-2-methylpyrazolo[1,5-d][1,2,4]triazin-4-yl)-3-fluorophenol;
2-(7-{[(3R)-1-ethylpiperidin-3-yl]amino}-2-methylpyrazolo[1,5-d][1,2,4]triazin-4-yl)-3-fluoro-5-methoxyphenol;
4-[2-(difluoromethoxy)-4-(trifluoromethyl)phenyl]-N-[(3R)-1-ethylpiperidin-3-yl]-2-methylpyrazolo[1,5-d][1,2,4]triazin-7-amine;
5-chloro-2-(4-{[(3R,5R)-5-fluoro-1-methylpiperidin-3-yl]amino}imidazo[1,5-d][1,2,4]triazin-1-yl)phenol;
5-chloro-2-(4-{[(3R)-1-(2,2,2-trifluoroethyl)piperidin-3-yl]amino}imidazo[1,5-d][1,2,4]triazin-1-yl)phenol;
5-chloro-2-(4-{[(3R)-1-(2,2-difluoroethyl)piperidin-3-yl]amino}imidazo[1,5-d][1,2,4]triazin-1-yl)phenol;
3-methyl-2-(2-methyl-7-{[(3R)-1-methylpiperidin-3-yl]amino}pyrazolo[1,5-d][1,2,4]triazin-4-yl)-5-(trifluoromethyl)phenol;
5-chloro-2-(4-{[(3R)-oxolan-3-yl]amino}imidazo[1,5-d][1,2,4]triazin-1-yl)phenol;
3-fluoro-2-(4-{[(1s,3s)-3-hydroxy-3-methylcyclobutyl]amino}pyrrolo[1,2-d][1,2,4]triazin-1-yl)-5-methoxyphenol;
5-ethyl-3-fluoro-2-(4-{[(3R)-1-methylpiperidin-3-yl]amino}pyrrolo[1,2-d][1,2,4]triazin-1-yl)phenol;
5-bromo-2-(4-{[(3R)-1-ethylpiperidin-3-yl]amino}pyrrolo[1,2-d][1,2,4]triazin-1-yl)phenol;
1-[2-(difluoromethoxy)-4-(trifluoromethoxy)phenyl]-N-[(3R)-1-ethylpiperidin-3-yl]pyrrolo[1,2-d][1,2,4]triazin-4-amine;
1-[2-(difluoromethoxy)-4-(trifluoromethyl)phenyl]-N-[(3R)-1-ethylpiperidin-3-yl]-8-fluoropyrrolo[1,2-d][1,2,4]triazin-4-amine;
2-{4-[(1-methylpiperidin-3-yl)methyl]pyrido[3,4-d]pyridazin-1-yl}-5-(trifluoromethyl)phenol;
1-[2-(difluoromethoxy)-4-(trifluoromethyl)phenyl]-N-(2-methoxy-2-methylpropyl)pyrrolo[1,2-d][1,2,4]triazin-4-amine;
3-fluoro-5-methyl-2-(4-{[(3R)-1-(propan-2-yl)piperidin-3-yl]amino}pyrrolo[1,2-d][1,2,4]triazin-1-yl)phenol;
3-fluoro-5-methyl-2-(4-{[(3R)-1-(2,2,2-trifluoroethyl)piperidin-3-yl]amino}pyrrolo[1,2-d][1,2,4]triazin-1-yl)phenol;
2-(4-{[(3R,5R)-1-ethyl-5-fluoropiperidin-3-yl]amino}pyrrolo[1,2-d][1,2,4]triazin-1-yl)-3-fluoro-5-methylphenol;
2-(4-{[(3R,5R)-1-ethyl-5-fluoropiperidin-3-yl]amino}pyrrolo[1,2-d][1,2,4]triazin-1-yl)-5-(trifluoromethoxy)phenol;
(1s,3s)-3-({1-[2,4-bis(difluoromethoxy)phenyl]pyrrolo[1,2-d][1,2,4]triazin-4-yl}amino)-1-methylcyclobutan-1-ol;
1-[2-(difluoromethoxy)-4-(trifluoromethyl)phenyl]-N-[(3R)-1-ethylpiperidin-3-yl]imidazo[1,5-d][1,2,4]triazin-4-amine;
2-(4-{[(3R,5S)-5-fluoropiperidin-3-yl]amino}pyrrolo[1,2-d][1,2,4]triazin-1-yl)-5-(trifluoromethyl)phenol;
5-cyclopropyl-2-(4-{[(3R,5R)-1-ethyl-5-fluoropiperidin-3-yl]amino}pyrrolo[1,2-d][1,2,4]triazin-1-yl)-3-fluorophenol;
5-chloro-3-fluoro-2-(4-{[(2S)-2-hydroxypropyl]amino}pyrrolo[1,2-d][1,2,4]triazin-1-yl)phenol;
5-chloro-2-(4-{[(1R,3S)-3-hydroxycyclohexyl]amino}imidazo[1,5-d][1,2,4]triazin-1-yl)phenol;
5-chloro-2-(7-{[(3R)-1-ethylpiperidin-3-yl]amino}-2-methylpyrazolo[1,5-d][1,2,4]triazin-4-yl)-3-fluorophenol;
5-chloro-2-(4-{[(3S)-oxolan-3-yl]amino}imidazo[1,5-d][1,2,4]triazin-1-yl)phenol;
5-chloro-2-(4-{[(1R,3S)-3-methoxycyclohexyl]amino}imidazo[1,5-d][1,2,4]triazin-1-yl)phenol;
2-[4-({[(2S)-1-ethylpyrrolidin-2-yl]methyl}amino)pyrrolo[1,2-d][1,2,4]triazin-1-yl]-3-fluoro-5-methylphenol;
5-cyclopropyl-2-[4-({[(2S)-1-ethylpyrrolidin-2-yl]methyl}amino)pyrrolo[1,2-d][1,2,4]triazin-1-yl]-3-fluorophenol;
2-(7-{[(3R)-1-ethylpiperidin-3-yl]amino}-2-methylpyrazolo[1,5-d][1,2,4]triazin-4-yl)-5-(trifluoromethoxy)phenol;
4-[2,4-bis(trifluoromethyl)phenyl]-N-[(3R)-1-ethylpiperidin-3-yl]-2-methylpyrazolo[1,5-d][1,2,4]triazin-7-amine;
5-cyclopropyl-2-(4-{[(2R)-2-hydroxypropyl]amino}pyrrolo[1,2-d][1,2,4]triazin-1-yl)phenol;
2-(4-{[(2R)-2-hydroxypropyl]amino}pyrrolo[1,2-d][1,2,4]triazin-1-yl)-5-methoxyphenol;
2-(4-{[(2R)-2-hydroxypropyl]amino}pyrrolo[1,2-d][1,2,4]triazin-1-yl)-5-(trifluoromethyl)phenol;
1-[2,4-bis(trifluoromethyl)phenyl]-N-{[(3S)-oxolan-3-yl]methyl}pyrrolo[1,2-d][1,2,4]triazin-4-amine;
1-[2,4-bis(trifluoromethyl)phenyl]-N-{[(3R)-oxolan-3-yl]methyl}pyrrolo[1,2-d][1,2,4]triazin-4-amine;
1-[2-(difluoromethoxy)-4-(trifluoromethyl)phenyl]-7-methyl-N-[(3R)-1-methylpiperidin-3-yl]pyrrolo[1,2-d][1,2,4]triazin-4-amine;
1-[2-(difluoromethoxy)-4-(trifluoromethyl)phenyl]-8-fluoro-N-[(3R)-1-(propan-2-yl)piperidin-3-yl]pyrrolo[1,2-d][1,2,4]triazin-4-amine;
2-(8-fluoro-4-{[(1s,3s)-3-hydroxy-3-methylcyclobutyl]amino}pyrrolo[1,2-d][1,2,4]triazin-1-yl)-5-(trifluoromethyl)phenol;
2-(4-{[(3R)-1-ethylpiperidin-3-yl]amino}-8-fluoropyrrolo[1,2-d][1,2,4]triazin-1-yl)-5-(trifluoromethyl)phenol;
2-(8-fluoro-4-{[(2S)-2-hydroxypropyl]amino}pyrrolo[1,2-d][1,2,4]triazin-1-yl)-5-(trifluoromethyl)phenol;
2-(8-fluoro-4-{[(3R,5R)-5-fluoro-1-methylpiperidin-3-yl]amino}pyrrolo[1,2-d][1,2,4]triazin-1-yl)-5-(trifluoromethyl)phenol;
(3R,5R)-5-({8-fluoro-1-[2-hydroxy-4-(trifluoromethyl)phenyl]pyrrolo[1,2-d][1,2,4]triazin-4-yl}amino)-1-methylpiperidin-3-ol;
2-(4-{[(3R,5R)-1-ethyl-5-fluoropiperidin-3-yl]amino}-8-fluoropyrrolo[1,2-d][1,2,4]triazin-1-yl)-5-(trifluoromethoxy)phenol;
5-cyclopropyl-2-(8-fluoro-4-{[(3R)-1-methylpiperidin-3-yl]amino}pyrrolo[1,2-d][1,2,4]triazin-1-yl)phenol;
5-cyclopropyl-2-(4-{[(3R)-1-ethylpiperidin-3-yl]amino}-8-fluoropyrrolo[1,2-d][1,2,4]triazin-1-yl)phenol;
5-cyclopropyl-3-fluoro-2-(8-fluoro-4-{[(3R)-1-methylpiperidin-3-yl]amino}pyrrolo[1,2-d][1,2,4]triazin-1-yl)phenol;
5-cyclopropyl-2-(4-{[(3R)-1-ethylpiperidin-3-yl]amino}-8-fluoropyrrolo[1,2-d][1,2,4]triazin-1-yl)-3-fluorophenol;
(3R,5R)-5-({1-[2-(difluoromethoxy)-4-(trifluoromethyl)phenyl]-8-fluoropyrrolo[1,2-d][1,2,4]triazin-4-yl}amino)-1-ethylpiperidin-3-ol;
2-(4-{[(3R)-1-ethylpiperidin-3-yl]amino}-8-fluoropyrrolo[1,2-d][1,2,4]triazin-1-yl)-5-(trifluoromethoxy)phenol;
(3R,5R)-1-ethyl-5-({8-fluoro-1-[2-hydroxy-4-(trifluoromethoxy)phenyl]pyrrolo[1,2-d][1,2,4]triazin-4-yl}amino)piperidin-3-ol;
2-(4-{[(3R)-1-ethylpiperidin-3-yl]amino}pyrrolo[1,2-d][1,2,4]triazin-1-yl)-3-methyl-5-(trifluoromethyl)phenol;
3-fluoro-2-(7-methyl-4-{[(3R)-1-methylpiperidin-3-yl]amino}pyrrolo[1,2-d][1,2,4]triazin-1-yl)-5-(trifluoromethyl)phenol;
5-chloro-3-fluoro-2-(4-{[(3R)-oxan-3-yl]amino}pyrrolo[1,2-d][1,2,4]triazin-1-yl)phenol;
2-(4-{[(3R)-1-ethylpiperidin-3-yl]amino}pyrrolo[1,2-d][1,2,4]triazin-1-yl)-5-(trifluoromethoxy)phenol;
1-[2-(difluoromethoxy)-4-(trifluoromethyl)phenyl]-N-{[(2S)-oxolan-2-yl]methyl}pyrrolo[1,2-d][1,2,4]triazin-4-amine;
1-[2-(difluoromethoxy)-4-(trifluoromethyl)phenyl]-N-{[(2R)-oxolan-2-yl]methyl}pyrrolo[1,2-d][1,2,4]triazin-4-amine;
1-[2,4-bis(difluoromethoxy)phenyl]-N-[(3R)-1-methylpiperidin-3-yl]pyrrolo[1,2-d][1,2,4]triazin-4-amine;
1-[2,4-bis(difluoromethoxy)phenyl]-N-[(3R)-1-ethylpiperidin-3-yl]pyrrolo[1,2-d][1,2,4]triazin-4-amine;
2-[(3R)-3-({1-[2,4-bis(difluoromethoxy)phenyl]pyrrolo[1,2-d][1,2,4]triazin-4-yl}amino)piperidin-1-yl]ethan-1-ol;
1-[2,4-bis(difluoromethoxy)phenyl]-N-[(3R,5R)-5-fluoro-1-methylpiperidin-3-yl]pyrrolo[1,2-d][1,2,4]triazin-4-amine;
5-(difluoromethoxy)-2-(4-{[(3R)-1-ethylpiperidin-3-yl]amino}pyrrolo[1,2-d][1,2,4]triazin-1-yl)-3-fluorophenol;
5-(difluoromethoxy)-3-fluoro-2-(4-{[(3R,5R)-5-fluoro-1-methylpiperidin-3-yl]amino}pyrrolo[1,2-d][1,2,4]triazin-1-yl)phenol;
5-(difluoromethoxy)-3-fluoro-2-(4-{[(1s,3s)-3-hydroxy-3-methylcyclobutyl]amino}pyrrolo[1,2-d][1,2,4]triazin-1-yl)phenol;
5-(difluoromethoxy)-2-(4-{[(3R,5R)-1-ethyl-5-fluoropiperidin-3-yl]amino}pyrrolo[1,2-d][1,2,4]triazin-1-yl)-3-fluorophenol;
5-chloro-2-(4-{[(3R,5R)-1-ethyl-5-fluoropiperidin-3-yl]amino}pyrrolo[1,2-d][1,2,4]triazin-1-yl)-3-fluorophenol;
5-chloro-3-fluoro-2-(4-{[(oxolan-2-yl)methyl]amino}pyrrolo[1,2-d][1,2,4]triazin-1-yl)phenol;
5-bromo-2-(4-{[(3R)-1-(2-hydroxyethyl)piperidin-3-yl]amino}imidazo[1,5-d][1,2,4]triazin-1-yl)phenol;
1-[2-(difluoromethoxy)-4-(trifluoromethyl)phenyl]-N-[(3R)-1-methylpiperidin-3-yl]imidazo[1,5-d][1,2,4]triazin-4-amine;
1-[2-(difluoromethoxy)-4-(trifluoromethyl)phenyl]-N-[(3R,5R)-5-fluoro-1-methylpiperidin-3-yl]imidazo[1,5-d][1,2,4]triazin-4-amine;
2-(4-{[(3R)-1-ethylpiperidin-3-yl]amino}imidazo[1,5-d][1,2,4]triazin-1-yl)-3-fluoro-5-methylphenol;
5-bromo-2-(4-{[(1s,3s)-3-hydroxy-3-methylcyclobutyl]amino}imidazo[1,5-d][1,2,4]triazin-1-yl)phenol;
5-bromo-2-(4-{[(3R)-1-ethylpiperidin-3-yl]amino}imidazo[1,5-d][1,2,4]triazin-1-yl)phenol;
5-ethoxy-3-fluoro-2-(4-{[(1s,3s)-3-hydroxy-3-methylcyclobutyl]amino}pyrrolo[1,2-d][1,2,4]triazin-1-yl)phenol;
5-ethoxy-3-fluoro-2-(4-{[(3R)-1-methylpiperidin-3-yl]amino}pyrrolo[1,2-d][1,2,4]triazin-1-yl)phenol;
5-ethoxy-3-fluoro-2-(4-{[(3R,5R)-5-fluoro-1-methylpiperidin-3-yl]amino}pyrrolo[1,2-d][1,2,4]triazin-1-yl)phenol;
2-(7-{[(3R)-1-ethylpiperidin-3-yl]amino}pyrazolo[1,5-d][1,2,4]triazin-4-yl)-5-methoxyphenol;
2-(7-{[(3R)-1-ethylpiperidin-3-yl]amino}pyrazolo[1,5-d][1,2,4]triazin-4-yl)-5-methylphenol;
5-methoxy-2-(7-{[(3R)-1-methylpiperidin-3-yl]amino}pyrazolo[1,5-d][1,2,4]triazin-4-yl)phenol;
(R)-2-(4-((1-ethylpiperidin-3-yl)amino)pyrrolo[1,2-d][1,2,4]triazin-1-yl)-3, 5-dimethylphenol;
(3S,5R)-5-((1-(2-fluoro-6-hydroxy-4-methylphenyl)pyrrolo[1,2-d][1,2,4]triazin-4-yl)amino)-1-methylpiperidin-3-ol;
(3S,5R)-1-ethyl-5-((1-(2-fluoro-6-hydroxy-4-methylphenyl)pyrrolo[1,2-d][1,2,4]triazin-4-yl)amino)piperidin-3-ol;
(3S,5R)-5-((1-(4-cyclopropyl-2-fluoro-6-hydroxyphenyl)pyrrolo[1,2-d][1,2,4]triazin-4-yl)amino)-1-methylpiperidin-3-ol; and
(3S,5R)-5-((1-(4-cyclopropyl-2-fluoro-6-hydroxyphenyl)pyrrolo[1,2-d][1,2,4]triazin-4-yl)amino)-1-ethylpiperidin-3-ol;
wherein a form of the compound may be selected from the group consisting of a pharmaceutically acceptable salt, hydrate, solvate, racemate, enantiomer, diastereomer, stereoisomer, and tautomer form thereof.

15. A compound or form thereof, selected from the group consisting of:
2-(4-{[(3R)-1-methylpiperidin-3-yl]amino}phthalazin-1-yl)-5-(trifluoromethyl)phenol formate;
2-(4-{[(3R)-1-methylpiperidin-3-yl]amino}-5,6,7,8-tetrahydrophthalazin-1-yl)-5-(trifluoromethyl)phenol formate;
2-(4-{[(3R)-1-methylpiperidin-3-yl]amino}-6,7-dihydro-5H-cyclopenta[d]pyridazin-1-yl)-5-(trifluoromethyl)phenol formate;
2-{4-[(pyrrolidin-3-yl)amino]phthalazin-1-yl}-5-(trifluoromethyl)phenol formate;
2-(4-{[(3R)-piperidin-3-yl]amino}phthalazin-1-yl)-5-(trifluoromethyl)phenol hydrochloride;
2-(8-methyl-4-{[(3R)-piperidin-3-yl]amino}-5,6,7,8-tetrahydrophthalazin-1-yl)-5-(trifluoromethyl)phenol formate;
2-{4-[(piperidin-3-yl)methyl]phthalazin-1-yl}-5-(trifluoromethyl)phenol hydrochloride;
2-(4-{[(3R)-piperidin-3-yl]amino}pyrido[3,4-d]pyridazin-1-yl)-5-(trifluoromethyl)phenol diformate;
2-(4-{[(3R)-1-ethylpiperidin-3-yl]amino}pyrido[3,4-d]pyridazin-1-yl)-5-(trifluoromethyl)phenol formate;
2-{4-[(1-methylpiperidin-3-yl)methyl]phthalazin-1-yl}-5-(trifluoromethyl)phenol formate;
2-(4-{[(3R)-1-(2-hydroxyethyl)piperidin-3-yl]amino}phthalazin-1-yl)-5-(trifluoromethyl)phenol formate;
(3S,5R)-5-({4-[2-hydroxy-4-(trifluoromethyl)phenyl]phthalazin-1-yl}amino)piperidin-3-ol formate;
2-(1-{[(3R)-1-methylpiperidin-3-yl]amino}pyrido[3,4-d]pyridazin-4-yl)-5-(trifluoromethyl)phenol N-ethylethanamine;
2-(4-{[(3R)-1-(2-hydroxyethyl)piperidin-3-yl]amino}pyrido[3,4-d]pyridazin-1-yl)-5-(trifluoromethyl)phenol diformate;
(3S,5R)-5-({4-[2-hydroxy-4-(trifluoromethyl)phenyl]phthalazin-1-yl}amino)-1-methylpiperidin-3-ol formate;
2-(5-{[(3R)-1-methylpiperidin-3-yl]amino}pyrido[2,3-d]pyridazin-8-yl)-5-(trifluoromethyl)phenol formate;
(3R,5R)-5-({4-[2-hydroxy-4-(trifluoromethyl)phenyl]phthalazin-1-yl}amino)piperidin-3-ol formate;
2-(4-{[(3R)-1-methylpiperidin-3-yl]amino}furo[2,3-d]pyridazin-7-yl)-5-(trifluoromethyl)phenol formate;
5-methyl-2-(8-{[(3R)-1-methylpiperidin-3-yl]amino}pyrido[2,3-d]pyridazin-5-yl)phenol formate;
[(3R)-3-({4-[2-hydroxy-4-(trifluoromethyl)phenyl]phthalazin-1-yl}amino)piperidin-1-yl]acetic acid formate;
2-{1-[(piperidin-3-yl)methyl]pyrido[3,4-d]pyridazin-4-yl}-5-(trifluoromethyl)phenol hydrochloride;
2-(4-{[(3R)-1-methylpiperidin-3-yl]amino}phthalazin-1-yl)-5-(1-methylpyrrolidin-3-yl)phenol formate;
2-[1-({[(2S)-pyrrolidin-2-yl]methyl}amino)pyrido[3,4-d]pyridazin-4-yl]-5-(trifluoromethyl)phenol formate;
2-[4-({[(2S)-pyrrolidin-2-yl]methyl}amino)pyrido[3,4-d]pyridazin-1-yl]-5-(trifluoromethyl)phenol formate;
2-[4-({[(2S)-1-methylpyrrolidin-2-yl]methyl}amino)pyrido[3,4-d]pyridazin-1-yl]-5-(trifluoromethyl)phenol formate;
2-[4-({[(2S)-1-ethylpyrrolidin-2-yl]methyl}amino)pyrido[3,4-d]pyridazin-1-yl]-5-(trifluoromethyl)phenol formate;
2-{1-[(1-methylpiperidin-3-yl)methyl]pyrido[3,4-d]pyridazin-4-yl}-5-(trifluoromethyl)phenol formate;
1-[2-methoxy-4-(trifluoromethyl)phenyl]-N-[(3R)-1-methylpiperidin-3-yl]pyrido[3,4-d]pyridazin-4-amine formate;
4-[2-(difluoromethoxy)-4-(trifluoromethyl)phenyl]-N-[(3R)-1-methylpiperidin-3-yl]phthalazin-1-amine formate;
1-[2-(difluoromethoxy)-4-(trifluoromethyl)phenyl]-N-[(3R)-1-methylpiperidin-3-yl]pyrido[3,4-d]pyridazin-4-amine formate;
4-[2-(difluoromethoxy)-4-(trifluoromethyl)phenyl]-N-[(3R)-1-methylpiperidin-3-yl]pyrido[3,4-d]pyridazin-1-amine formate;
4-[2-amino-4-(trifluoromethyl)phenyl]-N-[(3R)-1-methylpiperidin-3-yl]phthalazin-1-amine formate;
N-[2-(4-{[(3R)-1-methylpiperidin-3-yl]amino}phthalazin-1-yl)-5-(trifluoromethyl)phenyl]methanesulfonamide formate;
4-[2-(methylamino)-4-(trifluoromethyl)phenyl]-N-[(3R)-1-methylpiperidin-3-yl]phthalazin-1-amine formate;
N-[2-(4-{[(3R)-1-methylpiperidin-3-yl]amino}phthalazin-1-yl)-5-(trifluoromethyl)phenyl]acetamide formate;
1-[2-fluoro-4-(trifluoromethyl)phenyl]-N-[(3R)-1-methylpiperidin-3-yl]pyrido[3,4-d]pyridazin-4-amine formate;
1-(4-chloro-2-fluorophenyl)-N-[(3R)-1-methylpiperidin-3-yl]pyrido[3,4-d]pyridazin-4-amine formate;
1-[4-chloro-2-(difluoromethoxy)phenyl]-N-[(3R)-1-methylpiperidin-3-yl]pyrido[3,4-d]pyridazin-4-amine formate;
4-[4-chloro-2-(difluoromethoxy)phenyl]-N-[(3R)-1-methylpiperidin-3-yl]phthalazin-1-amine formate;
1-[2-(difluoromethoxy)-4-methylphenyl]-N-[(3R)-1-methylpiperidin-3-yl]pyrido[3,4-d]pyridazin-4-amine formate;
4-[2-(difluoromethoxy)-4-methylphenyl]-N-[(3R)-1-methylpiperidin-3-yl]phthalazin-1-amine formate;
1-[2-chloro-4-(trifluoromethyl)phenyl]-N-[(3R)-1-methylpiperidin-3-yl]pyrido[3,4-d]pyridazin-4-amine formate;
4-[2-methoxy-4-(trifluoromethyl)phenyl]-1-methyl-N-[(3R)-1-methylpiperidin-3-yl]-1H-pyrazolo[3,4-d]pyridazin-7-amine formate;
2-(1-methyl-7-{[(3R)-1-methylpiperidin-3-yl]amino}-1H-pyrazolo[3,4-d]pyridazin-4-yl)-5-(trifluoromethyl)phenol formate;
2-(1-methyl-8-{[(3R)-1-methylpiperidin-3-yl]amino}-1,2,3,4-tetrahydropyrido[2,3-d]pyridazin-5-yl)-5-(trifluoromethyl)phenol triformate;
5-chloro-2-(4-{[(3R)-1-methylpiperidin-3-yl]amino}pyrido[3,4-d]pyridazin-1-yl)benzonitrile formate;
2-amino-3-chloro-6-(4-{[(3R)-1-methylpiperidin-3-yl]amino}phthalazin-1-yl)phenol formate;
4-(2,3-difluoro-4-methylphenyl)-N-[(3R)-1-methylpiperidin-3-yl]phthalazin-1-amine formate;
5-methoxy-2-(4-{[(3R)-1-methylpiperidin-3-yl]amino}pyrido[3,4-d]pyridazin-1-yl)phenol formate;
1-[2,4-bis(trifluoromethyl)phenyl]-N-[(3R)-1-methylpiperidin-3-yl]pyrido[3,4-d]pyridazin-4-amine formate;
1-[4-methoxy-2-(trifluoromethyl)phenyl]-N-[(3R)-1-methylpiperidin-3-yl]pyrido[3,4-d]pyridazin-4-amine formate;
2-(4-{[(3R)-1-methylpiperidin-3-yl]amino}pyrido[3,4-d]pyridazin-1-yl)-5-(trifluoromethyl)benzamide formate;
5-[2-(difluoromethoxy)-4-(trifluoromethyl)phenyl]-N-[(3R)-1-methylpiperidin-3-yl]pyrido[2,3-d]pyridazin-8-amine formate;
8-[2-(difluoromethoxy)-4-(trifluoromethyl)phenyl]-N-[(3R)-1-methylpiperidin-3-yl]pyrido[2,3-d]pyridazin-5-amine formate;
2-{1-[(1-methylazepan-3-yl)amino]pyrido[3,4-d]pyridazin-4-yl}-5-(trifluoromethyl)phenol formate;
2-(4-{[(3R)-1-methylazepan-3-yl]amino}pyrido[3,4-d]pyridazin-1-yl)-5-(trifluoromethyl)phenol formate;
N-[(3R)-1-methylpiperidin-3-yl]-1-[2-(1-methyl-1H-pyrazol-4-yl)-4-(trifluoromethyl)phenyl]pyrido[3,4-d]pyridazin-4-amine formate;
[2-(4-{[(3R)-1-methylpiperidin-3-yl]amino}pyrido[3,4-d]pyridazin-1-yl)-5-(trifluoromethyl)phenyl]methanol formate;
N-[(3R)-1-methylpiperidin-3-yl]-1-(2,4,6-trimethylphenyl)pyrido[3,4-d]pyridazin-4-amine formate;
2-[(3R)-3-({1-[2-(difluoromethoxy)-4-(trifluoromethyl)phenyl]pyrido[3,4-d]pyridazin-4-yl}amino)piperidin-1-yl]ethan-1-ol formate;
6-chloro-3-fluoro-2-(4-{[(3R)-1-methylpiperidin-3-yl]amino}pyrido[3,4-d]pyridazin-1-yl)phenol formate;
6-chloro-3-fluoro-2-(5-{[(3R)-1-methylpiperidin-3-yl]amino}pyrido[2,3-d]pyridazin-8-yl)phenol formate;
N-[(3R)-1-methylpiperidin-3-yl]-1-[2-(2,2,2-trifluoroethyl)-4-(trifluoromethyl)phenyl]pyrido[3,4-d]pyridazin-4-amine formate;
3-methyl-4-(4-{[(3R)-1-methylpiperidin-3-yl]amino}pyrido[3,4-d]pyridazin-1-yl)benzonitrile formate;
1-(4-chloro-2,6-dimethylphenyl)-N-[(3R)-1-methylpiperidin-3-yl]pyrido[3,4-d]pyridazin-4-amine formate;
2,2,2-trifluoro-1-[3-hydroxy-4-(4-{[(3R)-piperidin-3-yl]amino}phthalazin-1-yl)phenyl]ethan-1-one formate;
5-(cyclopropylethynyl)-2-(4-{[(3R)-1-methylpiperidin-3-yl]amino}phthalazin-1-yl)phenol formate;
2-(4-{[(3R)-1-methylpiperidin-3-yl]amino}phthalazin-1-yl)-5-(prop-1-yn-1-yl)phenol formate;
N-[(3R)-1-methylpiperidin-3-yl]-1-[2-(prop-1-yn-1-yl)-4-(trifluoromethyl)phenyl]pyrido[3,4-d]pyridazin-4-amine formate;
2-{1-[(1,4-oxazepan-6-yl)amino]pyrido[3,4-d]pyridazin-4-yl}-5-(trifluoromethyl)phenol formate;
4-[2-(difluoromethoxy)-4-(trifluoromethyl)phenyl]-N-[(3R)-piperidin-3-yl]pyrido[3,4-d]pyridazin-1-amine formate;
4-[2-(difluoromethoxy)-4-(trifluoromethyl)phenyl]-N-[(3R)-1-ethylpiperidin-3-yl]pyrido[3,4-d]pyridazin-1-amine formate;
4-[2-(difluoromethoxy)-4-(trifluoromethyl)phenyl]-N-[(3R)-1-methylpiperidin-3-yl]-6,7-dihydro-5H-cyclopenta[d]pyridazin-1-amine formate;
5-(cyclopropyloxy)-2-(4-{[(3R)-1-methylpiperidin-3-yl]amino}pyrido[3,4-d]pyridazin-1-yl)phenol formate;
2-[(3R)-3-({4-[2-(difluoromethoxy)-4-(trifluoromethyl)phenyl]pyrido[3,4-d]pyridazin-1-yl}amino)piperidin-1-yl]ethan-1-ol formate;
4-[2-(difluoromethoxy)-4-methylphenyl]-N-[(3R)-1-methylpiperidin-3-yl]-6,7-dihydro-5H-cyclopenta[d]pyridazin-1-amine formate;
4-[2-(difluoromethoxy)phenyl]-N-[(3R)-1-methylpiperidin-3-yl]-6,7-dihydro-5H-cyclopenta[d]pyridazin-1-amine formate;
4-[4-cyclopropyl-2-(difluoromethoxy)phenyl]-N-[(3R)-1-methylpiperidin-3-yl]-6,7-dihydro-5H-cyclopenta[d]pyridazin-1-amine formate;
1-[4-cyclopropyl-2-(difluoromethoxy)phenyl]-N-[(3R)-1-methylpiperidin-3-yl]pyrido[3,4-d]pyridazin-4-amine formate;
1-[4-cyclopropyl-2-(difluoromethyl)phenyl]-N-[(3R)-1-methylpiperidin-3-yl]pyrido[3,4-d]pyridazin-4-amine formate;
N-[(3R)-azepan-3-yl]-1-[2-methoxy-4-(trifluoromethyl)phenyl]pyrido[3,4-d]pyridazin-4-amine formate;
2-{4-[(1,4-oxazepan-6-yl)amino]pyrido[3,4-d]pyridazin-1-yl}-5-(trifluoromethyl)phenol formate;
2-{4-[(azepan-4-yl)amino]pyrido[3,4-d]pyridazin-1-yl}-5-(trifluoromethyl)phenol formate;
2-(3-methyl-4-{[(3R)-1-methylpiperidin-3-yl]amino}[1,2]oxazolo[4,5-d]pyridazin-7-yl)-5-(trifluoromethyl)phenol formate;
2-(4-{[(3R)-1-(2-hydroxyethyl)piperidin-3-yl]amino}-3-methyl[1,2]oxazolo[4,5-d]pyridazin-7-yl)-5-(trifluoromethyl)phenol formate;
2-(4-{[(3R)-azepan-3-yl]amino}pyrido[3,4-d]pyridazin-1-yl)-5-(trifluoromethyl)phenol formate;
2-{4-[(1-methylazepan-4-yl)amino]pyrido[3,4-d]pyridazin-1-yl}-5-(trifluoromethyl)phenol formate;
2-{4-[(1,4-dimethyl-1,4-diazepan-6-yl)amino]pyrido[3,4-d]pyridazin-1-yl}-5-(trifluoromethyl)phenol formate;
ethyl (2S,5R)-5-({1-[2-hydroxy-4-(trifluoromethyl)phenyl]pyrido[3,4-d]pyridazin-4-yl}amino)piperidine-2-carboxylate hydrochloride;
5-methyl-2-(4-{[(3R)-1-methylpiperidin-3-yl]amino}pyrido[3,4-d]pyridazin-1-yl)benzene-1,3-diol formate;
2-(4-{[(1R,3S)-3-hydroxycyclohexyl]amino}pyrido[3,4-d]pyridazin-1-yl)-5-(trifluoromethyl)phenol formate;
2-(4-{[(1R,3R)-3-hydroxycyclohexyl]amino}pyrido[3,4-d]pyridazin-1-yl)-5-(trifluoromethyl)phenol formate;
3-fluoro-2-(4-{[(3R)-1-(2-hydroxyethyl)piperidin-3-yl]amino}pyrido[3,4-d]pyridazin-1-yl)-5-methylphenol formate;
2-(4-{[2-(dimethylamino)-2-methylpropyl]amino}pyrido[3,4-d]pyridazin-1-yl)-5-(trifluoromethyl)phenol formate;
N-{(3R)-1-[2-(difluoromethoxy)ethyl]piperidin-3-yl}-1-[2-methoxy-4-(trifluoromethyl)phenyl]pyrido[3,4-d]pyridazin-4-amine formate;
2-(4-{[(3 S,5S)-5-fluoro-1-methylpiperidin-3-yl]amino}pyrido[3,4-d]pyridazin-1-yl)-5-(trifluoromethyl)phenol formate;
2-(4-{[(3R,5R)-5-fluoro-1-methylpiperidin-3-yl]amino}pyrido[3,4-d]pyridazin-1-yl)-5-(trifluoromethyl)phenol formate;
5-chloro-3-fluoro-2-(4-{[(3R)-1-(2-hydroxyethyl)piperidin-3-yl]amino}pyrido[3,4-d]pyridazin-1-yl)phenol formate;
5-cyclopropyl-3-fluoro-2-(4-{[(3R)-1-(2-hydroxyethyl)piperidin-3-yl]amino}pyrido[3,4-d]pyridazin-1-yl)phenol formate;
3-fluoro-2-(4-{[(3R)-1-(2-hydroxyethyl)piperidin-3-yl]amino}pyrido[3,4-d]pyridazin-1-yl)phenol formate;
2-[4-({(3R)-1-[2-(difluoromethoxy)ethyl]piperidin-3-yl}amino)pyrido[3,4-d]pyridazin-1-yl]-5-(trifluoromethyl)phenol formate;
2-(4-{[(3R)-1-(propan-2-yl)piperidin-3-yl]amino}pyrido[3,4-d]pyridazin-1-yl)-5-(trifluoromethyl)phenol formate;
1-[2-(difluoromethyl)-4-methylphenyl]-N-[(3R)-1-methylpiperidin-3-yl]pyrido[3,4-d]pyridazin-4-amine formate;
2-(4-{[(1-methylpiperidin-4-yl)methyl]amino}pyrido[3,4-d]pyridazin-1-yl)-5-(trifluoromethyl)phenol formate;
2-[4-({[1-(2-hydroxyethyl)piperidin-4-yl]methyl}amino)pyrido[3,4-d]pyridazin-1-yl]-5-(trifluoromethyl)phenol formate;
2-(4-{[(3R,5R)-5-fluoropiperidin-3-yl]amino}pyrido[3,4-d]pyridazin-1-yl)-5-(trifluoromethyl)phenol formate;
5-ethyl-2-(4-{[(3R)-1-methylpiperidin-3-yl]amino}pyrido[3,4-d]pyridazin-1-yl)phenol formate;
N-{(3R)-1-[2-(difluoromethoxy)ethyl]piperidin-3-yl}-1-[2-(difluoromethoxy)-4-(trifluoromethyl)phenyl]pyrrolo[1,2-d][1,2,4]triazin-4-amine formate;
2-[(3R)-3-({1-[2-(difluoromethoxy)-4-(trifluoromethyl)phenyl]pyrrolo[1,2-d][1,2,4]triazin-4-yl}amino)piperidin-1-yl]ethan-1-ol formate;
2-(4-{[(3R)-1-methylpiperidin-3-yl]amino}imidazo[1,5-d][1,2,4]triazin-1-yl)-5-(trifluoromethyl)phenol formate;
1-[2-methoxy-4-(trifluoromethyl)phenyl]-N-[(3R)-1-methylpiperidin-3-yl]imidazo[1,5-d][1,2,4]triazin-4-amine formate;
2-(4-{[(3R)-1-ethylpiperidin-3-yl]amino}imidazo[1,5-d][1,2,4]triazin-1-yl)-5-(trifluoromethyl)phenol formate;
2-(4-{[(3R)-piperidin-3-yl]amino}pyrrolo[1,2-d][1,2,4]triazin-1-yl)-5-(trifluoromethoxy)phenol hydrochloride;
1-(4-chloro-2-methoxyphenyl)-N-[(3R)-1-methylpiperidin-3-yl]imidazo[1,5-d][1,2,4]triazin-4-amine formate;
5-chloro-2-(4-{[(3R)-1-methylpiperidin-3-yl]amino}imidazo[1,5-d][1,2,4]triazin-1-yl)phenol formate;
8-[2-methoxy-4-(trifluoromethyl)phenyl]-N-[(3R)-1-methylpiperidin-3-yl]imidazo[1,2-d][1,2,4]triazin-5-amine formate;
(3R,5R)-5-{[1-(4-cyclopropyl-2-fluoro-6-hydroxyphenyl)pyrrolo[1,2-d][1,2,4]triazin-4-yl]amino}-1-methylpiperidin-3-ol formate;
4-(4-bromo-2-methoxyphenyl)-N-[(3R)-piperidin-3-yl]pyrazolo[1,5-d][1,2,4]triazin-7-amine formate;
2-(4-{[(3R)-oxan-3-yl]amino}imidazo[1,5-d][1,2,4]triazin-1-yl)-5-(trifluoromethyl)phenol formate;
2-(5-{[(3R)-1-methylpiperidin-3-yl]amino}imidazo[1,2-d][1,2,4]triazin-8-yl)-5-(trifluoromethyl)phenol formate;
2-(4-{[(3R,5R)-5-fluoropiperidin-3-yl]amino}pyrrolo[1,2-d][1,2,4]triazin-1-yl)-5-(trifluoromethoxy)phenol hydrochloride;
4-[4-cyclopropyl-2-(trifluoromethoxy)phenyl]-2-methyl-N-[(3R)-1-methylpiperidin-3-yl]pyrazolo[1,5-d][1,2,4]triazin-7-amine formate;
2-methyl-N-[(3R)-1-methylpiperidin-3-yl]-4-[2-(trifluoromethoxy)phenyl]pyrazolo[1,5-d][1,2,4]triazin-7-amine formate;
2-(4-{[(2R)-2-hydroxypropyl]amino}pyrrolo[1,2-d][1,2,4]triazin-1-yl)-5-(trifluoromethoxy)phenol hydrochloride;
2-(8-fluoro-4-{[(3R)-1-methylpiperidin-3-yl]amino}pyrrolo[1,2-d][1,2,4]triazin-1-yl)-5-(trifluoromethoxy)phenol hydrochloride;
5-chloro-2-(4-{[(3R)-1-ethylpiperidin-3-yl]amino}imidazo[1,5-d][1,2,4]triazin-1-yl)phenol formate;
5-chloro-3-fluoro-2-(2-methyl-7-{[(3R)-1-methylpiperidin-3-yl]amino}pyrazolo[1,5-d][1,2,4]triazin-4-yl)phenol formate;
1-[2-(difluoromethoxy)-4-(trifluoromethyl)phenyl]-N-[(3S)-oxolan-3-yl]pyrrolo[1,2-d][1,2,4]triazin-4-amine formate;
2-(4-{[(3R)-1-methyl-1,2,3,6-tetrahydropyridin-3-yl]amino}imidazo[1,5-d][1,2,4]triazin-1-yl)-5-(trifluoromethyl)phenol formate;
2-(7-{[(3R)-1-methylpiperidin-3-yl]amino}[1,2,3]triazolo[1,5-d][1,2,4]triazin-4-yl)-5-(trifluoromethyl)phenol formate;
2-(4-{[(3S,4S)-4-fluoro-1-methylpiperidin-3-yl]amino}pyrrolo[1,2-d][1,2,4]triazin-1-yl)-5-(trifluoromethyl)phenol formate;
2-(4-{[(3R)-1-ethylpiperidin-3-yl]amino}pyrrolo[1,2-d][1,2,4]triazin-1-yl)-5-(trifluoromethyl)phenol formate;
2-(4-{[(3R)-1-(propan-2-yl)piperidin-3-yl]amino}pyrrolo[1,2-d][1,2,4]triazin-1-yl)-5-(trifluoromethyl)phenol formate;
1-[2-(difluoromethoxy)-6-fluoro-4-(trifluoromethyl)phenyl]-N-[(3R)-1-methylpiperidin-3-yl]pyrrolo[1,2-d][1,2,4]triazin-4-amine formate;
1-[4-chloro-2-(trifluoromethyl)phenyl]-N-[(3R)-1-methylpiperidin-3-yl]pyrrolo[1,2-d][1,2,4]triazin-4-amine formate;
5-cyclopropyl-2-(4-{[(3R)-1-(2,2-difluoroethyl)piperidin-3-yl]amino}pyrrolo[1,2-d][1,2,4]triazin-1-yl)-3-fluorophenol formate;
2-(4-{[(3R)-1-(2,2-difluoroethyl)piperidin-3-yl]amino}pyrrolo[1,2-d][1,2,4]triazin-1-yl)-3-fluoro-5-methylphenol formate;
2-(4-{[(3R,5R)-5-fluoro-1-methylpiperidin-3-yl]amino}imidazo[1,5-d][1,2,4]triazin-1-yl)-5-(trifluoromethyl)phenol formate;
2-(6-methyl-4-{[(3R)-1-methylpiperidin-3-yl]amino}imidazo[1,5-d][1,2,4]triazin-1-yl)-5-(trifluoromethyl)phenol formate;
5-bromo-2-(4-{[(3R)-1-(1-hydroxypropan-2-yl)piperidin-3-yl]amino}pyrrolo[1,2-d][1,2,4]triazin-1-yl)phenol formate;
5-cyclopropyl-2-(7-{[(3R)-1-ethylpiperidin-3-yl]amino}-2-methylpyrazolo[1,5-d][1,2,4]triazin-4-yl)-3-fluorophenol formate;
5-chloro-2-(4-{[(3R,5R)-5-fluoro-1-methylpiperidin-3-yl]amino}imidazo[1,5-d][1,2,4]triazin-1-yl)phenol formate;
5-ethyl-3-fluoro-2-(4-{[(3R)-1-methylpiperidin-3-yl]amino}pyrrolo[1,2-d][1,2,4]triazin-1-yl)phenol formate;
5-bromo-2-(4-{[(3R)-1-ethylpiperidin-3-yl]amino}pyrrolo[1,2-d][1,2,4]triazin-1-yl)phenol formate;
1-[2-(difluoromethoxy)-4-(trifluoromethyl)phenyl]-N-[(3R)-1-ethylpiperidin-3-yl]imidazo[1,5-d][1,2,4]triazin-4-amine formate;
2-[4-({[(2S)-1-ethylpyrrolidin-2-yl]methyl}amino)pyrrolo[1,2-d][1,2,4]triazin-1-yl]-3-fluoro-5-methylphenol formate;
5-cyclopropyl-2-[4-({[(2S)-1-ethylpyrrolidin-2-yl]methyl}amino)pyrrolo[1,2-d][1,2,4]triazin-1-yl]-3-fluorophenol formate;
2-(4-{[(3R)-1-ethylpiperidin-3-yl]amino}-8-fluoropyrrolo[1,2-d][1,2,4]triazin-1-yl)-5-(trifluoromethyl)phenol formate;
2-(8-fluoro-4-{[(3R,5R)-5-fluoro-1-methylpiperidin-3-yl]amino}pyrrolo[1,2-d][1,2,4]triazin-1-yl)-5-(trifluoromethyl)phenol formate;
(3R,5R)-5-({8-fluoro-1-[2-hydroxy-4-(trifluoromethyl)phenyl]pyrrolo[1,2-d][1,2,4]triazin-4-yl}amino)-1-methylpiperidin-3-ol formate;
2-(4-{[(3R,5R)-1-ethyl-5-fluoropiperidin-3-yl]amino}-8-fluoropyrrolo[1,2-d][1,2,4]triazin-1-yl)-5-(trifluoromethoxy)phenol formate;
5-bromo-2-(4-{[(3R)-1-(2-hydroxyethyl)piperidin-3-yl]amino}imidazo[1,5-d][1,2,4]triazin-1-yl)phenol formate;
1-[2-(difluoromethoxy)-4-(trifluoromethyl)phenyl]-N-[(3R)-1-methylpiperidin-3-yl]imidazo[1,5-d][1,2,4]triazin-4-amine formate; and
5-bromo-2-(4-{[(3R)-1-ethylpiperidin-3-yl]amino}imidazo[1,5-d][1,2,4]triazin-1-yl)phenol formate;
wherein a form of the compound may be selected from the group consisting of a hydrate, solvate, racemate, enantiomer, diastereomer, stereoisomer, and tautomer form thereof.

16. A pharmaceutical composition comprising a therapeutically effective amount of a compound according to any one of claims 1 to 15 or a pharmaceutically acceptable salt thereof and one or more pharmaceutically acceptable carriers.

17. A method for treating or ameliorating a disease modulated by NLRP3 in a subject in need thereof comprising, administering to the subject an effective amount of the compound according to of any one of claims 1 to 15.

18. A method of treating or ameliorating a disease modulated by NLRP3 according to claim 17 selected from Alzheimer disease, Frontotemporal dementia (FTD), Huntington's disease, Parkinson's disease, Perioperative neurocognitive disorders, Post-cardiac arrest cognitive impairment, Poststroke cognitive impairment, Sepsis, Sepsis associated encephalopathy, Subarachnoid hemorrhage, Macular Degeneration, Retinal neovascularization, Uveitis, Colitis, Endothelial dysfunction, Gout, Pseudogout, Graft-versus-host-disease (GvHD), Systemic lupus erythematosus-lupus nephritis, Cryopyrin-associated periodic syndromes (CAPS), Cystic fibrosis, Sickle-cell disease, VCP-associated disease, Liver fibrosis, Nonalcoholic fatty liver disease (NASH), muscle atrophy, inherited and acquired myopathies, e.g. Duchenne Muscular Dystrophy (DMD), Hyperalgesia, Multiple sclerosis-associated neuropathic pain, Acute Kidney Injury, Chronic crystal nephropathy, Chronic Kidney Disease, asthma and allergic airway inflammation Diabetes-associated atherosclerosis, Diabetic encephalopathy, Diabetic kidney disease, Islet transplantation rejection, Obesity-associated renal disease, Oxalate-induced nephropathy, Renal fibrosis, Renal hypertension, Type I diabetes, Type II diabetes, Psoriasis, Hidradenitis suppurativa, Atherosclerosis and Cytokine Release Syndrome (CRS).

19. The method of any one of claims 17 to 18, wherein the effective amount of the compound is in a range of from about 0.001 mg/kg/day to about 500 mg/kg/day.

20. A compound according to any one of claims 1 to 15 or a pharmaceutically acceptable salt thereof, for use in treating or ameliorating a disease modulated by NLRP3 selected from Alzheimer disease, Frontotemporal dementia (FTD), Huntington's disease, Parkinson's disease, Perioperative neurocognitive disorders, Post-cardiac arrest cognitive impairment, Poststroke cognitive impairment, Sepsis, Sepsis associated encephalopathy, Subarachnoid hemorrhage, Macular Degeneration, Retinal neovascularization, Uveitis, Colitis, Endothelial dysfunction, Gout, Pseudogout, Graft-versus-host-disease (GvHD), Systemic lupus erythematosus-lupus nephritis, Cryopyrin-associated periodic syndromes (CAPS), Cystic fibrosis, Sickle-cell disease, VCP-associated disease, Liver fibrosis, Nonalcoholic fatty liver disease (NASH), muscle atrophy, inherited and acquired myopathies, Hyperalgesia, Multiple sclerosis-associated neuropathic pain, Acute Kidney Injury, Chronic crystal nephropathy, Chronic Kidney Disease, asthma and allergic airway inflammation Diabetes-associated atherosclerosis, Diabetic encephalopathy, Diabetic kidney disease, Islet transplantation rejection, Obesity-associated renal disease, Oxalate-induced nephropathy, Renal fibrosis, Renal hypertension, Type I diabetes, Type II diabetes, Psoriasis, Hidradenitis suppurativa, Atherosclerosis and Cytokine Release Syndrome (CRS).

21. Use of a compound according to any one of claims 1 to 15, wherein the effective amount of the compound is in a range of from about 0.001 mg/kg/day to about 500 mg/kg/day.

22. Use of a compound according to any one of claims 1 to 15 in the preparation of a pharmaceutical composition for treating or ameliorating a disease modulated by NLRP3 in a subject in need thereof comprising, administering to the subject an effective amount of the compound or a form thereof in admixture with one or more of the pharmaceutically acceptable excipients.
